(19) United States
(12) Patent Application Publication
(10) Pub. No.: US 2025/0304577 A1
(43) Pub. Date: Oct. 2, 2025

(54) TYK2 INHIBITORS

(71) Applicant: BIOGEN MA INC., Cambridge, CA (US)

(72) Inventors: TeYu Chen, Charlestown, MA (US); Edward Yin Shiang Lin, Ashland, MA (US); Zhili Xin, Cambridge, MA (US); Tamara Halkina Levin, Belmont, MA (US); Nathan Edward Genung, Charlestown, MA (US); Jeffrey Vessels, Marlborough, MA (US); Lei Zhang, Westford, MA (US); Kurt D. van Vloten, Bellingham, MA (US); Kevin M. Guckian, Northborough, MA (US); Simone Sciabola, Wellesley, MA (US); Harold George Vandeveer, Stoneham, MA (US); Soma Maitra, Cambridge, MA (US); Marta Nevalainen, Holliston, MA (US); Jürgen Schulz, Bonn (DE); Felix Gonzalez Lopez de Turiso, San Diego, CA (US); Christopher Helal, Cambridge, MA (US)

(21) Appl. No.: 18/864,341

(22) PCT Filed: May 9, 2023
(86) PCT No.: PCT/US2023/021514

§ 371 Date: Nov. 8, 2024

Related U.S. Application Data
(60) Provisional application No. 63/340,142, filed on May 10, 2022.
(60) Provisional application No. 63/424,583, filed on Nov. 11, 2022.

(51) Int. Cl. C07D 471/04; A61K 31/437; A61K 31/444; A61K 31/4545; A61K 31/496; A61K 31/497; A61K 31/4995; A61K 31/501; A61K 31/506; A61K 31/53; A61K 31/5377; A61K 31/5383; A61K 31/5386; A61K 31/55; A61K 31/553; C07B 59/00; C07D 519/00

(52) U.S. Cl. CPC C07D 471/04; A61K 31/437; A61K 31/444; A61K 31/4545; A61K 31/496; A61K 31/497; A61K 31/4995; A61K 31/501; A61K 31/506; A61K 31/53; A61K 31/5377; A61K 31/5383; A61K 31/5386; A61K 31/55; A61K 31/553; C07B 59/002; C07D 519/00

(57) ABSTRACT

This disclosure relates to compounds of formula (I), or pharmaceutically acceptable salts thereof: in which all of the variables are as defined in the application. The compounds of the present disclosure are capable of inhibiting the activity of tyrosine kinase 2 (TYK2). The disclosure further provides methods of preparing the compounds of the disclosure, and methods for their therapeutic use.

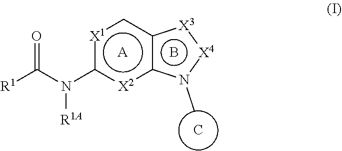

### RELATED APPLICATIONS

This application claims priority to U.S. Provisional Application No. 63/424,583 filed on Nov. 11, 2022 and U.S. Provisional Application No. 63/340,142, filed on May 10, 2022. The entire contents of each of the foregoing applications are expressly incorporated herein by reference.

### TECHNICAL FIELD

Provided are certain agents that target the degradation of Tyrosine kinase 2 (TYK2), and methods of making and using such agents

### BACKGROUND

Cytokines are small secreted proteins released by cells and have a specific effect on the interactions and communications between cells. Cytokine pathways mediate a broad range of biological functions including many aspects of inflammation and immunity through mostly extracellular signaling.

Tyrosine kinase 2 (TYK2) is a member of Janus kinases (JAK) that are cytoplasmic protein kinases associated with cytokine receptors and play a central role in mediating cytokine signaling (Kisseleva et al., Gene, 2002, 285, 1; and Yamaoka et al. Genome Biology 2004, 5, 253). The JAK family also includes JAK1, JAK2 and JAK3. More specifically, cytokine's engagement with cognate receptors triggers activation of receptors associate with JAK, which leads to JAK mediated tyrosine phosphorylation of signal transducer and activator of transcription (STAT) proteins and ultimately transcriptional activation of specific gene sets (Schindler et al, 2007, J. Biol. Chem. 282: 20059-63). Numerous cytokines known to activate the JAK family include the interferon (IFN) family (IFN-alpha, IFN-beta, IFN-omega, Limitin, IFN-gamma, IL-10, IL-19, IL-20, IL-22), the glycoprotein (gp) 130 family (IL-6, IL-11, OSM, LlF, CNTF, NNT-1/BSF-3, G-CSF, CT-1, Leptin, IL-12, IL-23), the gamma C family (IL-2, IL-7, TSLP, IL-9, IL-15, IL-21, IL-4, IL-13), IL-3 family (IL-3, IL-5, GM-CSF), the single chain family (EPO, GH, PRL, TPO), receptor tyrosine kinases (EGF, PDGF, CSF-1, HGF), and G-protein coupled receptors (AT1).

TYK2 is important in the signaling of the type I interferons (e.g., IFN-alpha), IL-6, IL-10, IL-12 and IL-23 (Liang, Y. et al., Expert Opinion on Therapeutic Targets, 2014, 18, 5, 571-580; Kisseleva et al., 2002, Gene 285:1-24; and Watford, W. T. & O'Shea, J. J., 2006, Immunity 25:695-697). Consistent with this, primary cells derived from a TYK2 deficient human are defective in type I interferon, IL-6, IL-10, IL-12 and IL-23 signaling. TYK2 signals with other members of the JAK family in the following combinations: TYK2/JAK1, TYK2/JAK2, TYK2/JAK1/JAK2.

Studies have shown that inappropriate JAK activities can arise from mutation, over-expression, or inappropriate regulation, dys-regulation or de-regulation, as well as over- or under-production of growth factors or cytokines, and therefore trigger a variety of biological cellular responses relating to cell growth, cell differentiation, cell function, survival, apoptosis, and cell mobility. The inappropriate JAK activities are implicated in many diseases that include but not limited to cancer, cardiovascular diseases, allergies, asthma and other respiratory diseases, autoimmune diseases, inflammatory diseases, bone diseases, metabolic disorders, and neurological and neurodegenerative disorders such as Alzheimer's disease.

Small molecule JAK inhibitors have emerged as a major therapeutic advancement in treating autoimmune diseases. To date, all known small molecule JAK inhibitors that have progressed into development are active site-directed inhibitors that bind to the adenosine triphosphate (ATP) site of the catalytic domain (also referred to as the JH1 or “Janus Homology 1” domain) of the JAK protein, which prevents catalytic activity of the kinase by blocking ATP, downstream phosphorylation, and resulting pathway signal transduction (Bryan et al., J. Med. Chem. 2018, 61, 9030-9058).

Because of the high homology of the ATP active site across the kinome and especially within the JAK family, it is a significant challenge to achieve high selectivity for a specific JAK family member while also maintaining selectivity within the kinome. As a result, many JAK inhibitors that have been developed are pan-JAK inhibitors or are modestly selective for one or more JAK family members. While these inhibitors have shown encouraging results in treating autoimmune diseases, undesirable side effects leading to a narrow therapeutic index have been observed and suggest the need for improved treatments.

TYK2 has been shown to be important in the differentiation and function of multiple cell types important in inflammatory disease and autoimmune disease including natural killer cells, B cells, and T helper cell types. Aberrant TYK2 expression is associated with multiple autoimmune or inflammatory conditions.

There remains a need for potent compounds that demonstrate high selectivity for TYK2 over other members of the JAK family.

### SUMMARY

One aspect of the present disclosure is a compound of formula (I):

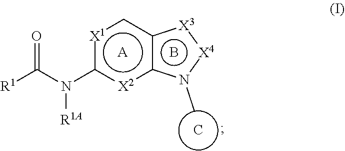

or a pharmaceutically acceptable salt thereof, wherein:
ring A is an aromatic or heteoaromatic ring fused with ring B that is a 5-membered heteroaromatic ring;
X¹ is N or CH;
X² is N or CR²;
X³ is N or CR³;
X⁴ is N or CR⁴;
ring C is phenyl, 5 or 6 membered monocyclic heterocyclyl, or 5 to 6 membered heteroaryl, each of which is optionally substituted by one or more R^{C};
each R^{C} is independently halo, —CN, —NR^{N1}R^{N2}, —NR^{N3}—C(O)—R⁷, —NR^{N4}—SO₂—R⁷, —C(O)—R⁷, —SO₂—R⁷, —OR^{O1}, C₁₋₆ alkyl, alkenyl, 3 to 7 membered monocyclic carbocyclyl, phenyl, 5 to 12 membered monocyclic or bicyclic heteroaryl, or 4 to 9 membered monocyclic or bicyclic heterocyclyl, wherein the C₁₋₆ alkyl, 3 to 7 membered monocyclic carbocyclyl, phenyl, 5 to 12 membered monocyclic or bicyclic heteroaryl, and 4 to 9 membered monocyclic or bicyclic heterocyclyl represented by R^{C} are each optionally substituted with one or more R^{C1}, or two R^{C} taken together with intervening atoms form a 3 to 7 membered monocyclic carbocyclyl optionally substituted with one or more halo;
each R^{C1} is independently halo, oxo, —CN, —OR^{O1}, —NR^{N1}R^{N2}, —C(O)—R⁷, —C(O)—OR^{O3}, —SO₂—R⁷, C₁₋₆ alkyl, C₃₋₆cycloalkyl, phenyl, 5 to 12 membered monocyclic or bicyclic heteroaryl, or 4 to 7 membered monocyclic heterocyclyl, wherein the C₁₋₆ alkyl, phenyl, 5 to 6 membered heteroaryl and 4 to 7 membered monocyclic heterocyclyl represented by R^{C1} are each optionally substituted by one or more substituents independently selected from halo, oxo, —CN, —OR^{O1}, —NR^{N1}R^{N2}, C₁₋₆ alkyl, C₁₋₄ haloalkyl, phenyl, 5 to 6 membered heteroaryl, 3 to 7 membered monocyclic carbocyclyl and 4 to 8 membered monocyclic heterocyclyl;
R¹ is H, C₁₋₆ alkyl, —OR^{1A}, —NR^{N1}R^{N2}, C₃₋₆ cycloalkyl, phenyl, 5 to 6 membered heteroaryl, 3 to 7 membered monocyclic carbocyclyl or 4 to 7 membered monocyclic heterocyclyl, wherein the C₁₋₆ alkyl, phenyl, C₃₋₆ cycloalkyl, 5 to 6 membered heteroaryl, 3 to 7 membered monocyclic carbocyclyl and 4 to 7 membered monocyclic heterocyclyl represented by R¹ are each optionally substituted by one or more R⁸;
R^{1A} is H or C₁₋₃alkyl;
or R¹ and R^{1A} together with the atom from which they are attached form a 5 or 6 membered monocyclic heterocycle;
R² is H or halo;
R³ is H, —NR^{N1}R^{N2}, —CN, halo, —C(O)—R⁷, —C(O)—OR^{O3}, —SO₂—R⁷, —OR^{O4}, C₁₋₆ alkyl, alkenyl, phenyl, 5 to 6 membered heteroaryl, 3 to 7 membered monocyclic carbocyclyl, or 4 to 9 membered monocyclic or bicyclic heterocyclyl, wherein the C₁₋₆ alkyl, phenyl, 5 to 6 membered heteroaryl, 3 to 7 membered monocyclic carbocyclyl and 4 to 9 membered monocyclic or bicyclic heterocyclyl represented by R³ are each optionally substituted by one or more R⁹;
R⁴ is H or halo;
each R⁷ is independently C₁₋₆ alkyl, phenyl, 5 to 6 membered heteroaryl, 3 to 7 membered monocyclic carbocyclyl or 4 to 7 membered monocyclic heterocyclyl; wherein the C₁₋₆ alkyl, phenyl, 5 to 6 membered heteroaryl, 3 to 7 membered monocyclic carbocyclyl and 4 to 7 membered monocyclic heterocyclyl represented by R⁷ are each optionally substituted by one or more substituents independently selected from halo, oxo, —CN, —OR^{O1}, —NR^{1a}R^{1b}, C₁₋₆ alkyl, C₁₋₄ haloalkyl, 3 to 7 membered monocyclic carbocyclyl and 4 to 7 membered monocyclic heterocyclyl;
each R⁸ is independently halo, oxo, —CN, —OR^{O1}, C₁₋₆ alkyl, C₁₋₄ haloalkyl, phenyl, 5 to 6 membered heteroaryl, 3 to 7 membered monocyclic carbocyclyl or 4 to 7 membered monocyclic heterocyclyl;
each R⁹ is independently halo, oxo, —OR^{O1}, —NR^{N1}R^{N2}, —CN, —C(O)—OR^{O3}, —SO₂—R¹⁰, C₁₋₆ alkyl, phenyl, 5 to 6 membered heteroaryl, 3 to 7 membered monocyclic carbocyclyl or 4 to 10 membered monocyclic or bicyclic heterocyclyl, wherein the C₁₋₆ alkyl, phenyl, 5 to 6 membered heteroaryl, 3 to 7 membered monocyclic carbocyclyl and 4 to 10 membered monocyclic or bicyclic heterocyclyl represented by R⁹ are each optionally substituted by one or more substituents independently selected from halo, oxo, —CN, —OR^{O1}, —NR^{N1}R^{N2}, C₁₋₆ alkyl, C₁₋₄ haloalkyl, phenyl, 5 to 6 membered heteroaryl, 3 to 7 membered monocyclic carbocyclyl and 4 to 7 membered monocyclic heterocyclyl;
each R^{O1} is independently H, C₁₋₆ alkyl, phenyl, 5 to 6 membered heteroaryl, 3 to 7 membered monocyclic or bicyclic carbocyclyl, or 4 to 7 membered monocyclic or bicyclic heterocyclyl, wherein the C₁₋₆ alkyl, phenyl, 5 to 6 membered heteroaryl, 3 to 7 membered monocyclic or bicyclic carbocyclyl, and 4 to 7 membered monocyclic or bicyclic heterocyclyl represented by R^{O1} are each optionally substituted by one or more R^{O2};
each R^{O2} is independently halo, OH, —CN, C₁₋₄ alkoxy, C₁₋₄ alkyl, phenyl, 5 to 6 membered heteroaryl, 3 to 7 membered monocyclic carbocylyl or 4 to 7 membered monocyclic or bicyclic heterocyclyl, wherein the C₁₋₄ alkyl, phenyl, 5 to 6 membered heteroaryl, 3 to 7 membered monocyclic carbocylyl and 4 to 7 membered monocyclic or bicyclic heterocyclyl are each optionally substituted with one or more halo, C₁₋₆ alkyl or —O—C₁₋₆alkyl;
each R^{O3} is independently H, C₁₋₆ alkyl, C₁₋₄ haloalkyl, phenyl, 5 to 6 membered heteroaryl, 3 to 7 membered monocyclic carbocyclyl or 4 to 7 membered monocyclic heterocyclyl, wherein the C₁₋₆ alkyl, phenyl, 5 to 6 membered heteroaryl, 3 to 7 membered monocyclic carbocyclyl and 4 to 7 membered monocyclic heterocyclyl represented by R^{O3} are each optionally substituted by one or more R^{O2};
R^{O4} is H, C₁₋₆ alkyl, phenyl, 5 to 6 membered heteroaryl, 3 to 7 membered monocyclic carbocyclyl or 4 to 7 membered monocyclic heterocyclyl, wherein the C₁₋₆ alkyl, phenyl, 5 to 6 membered heteroaryl, 3 to 7 membered monocyclic carbocyclyl and 4 to 7 membered monocyclic heterocyclyl represented by R^{O4} are each optionally substituted by one or more substituents independently selected from halo, oxo, —CN, —OR^{O1}, —NR^{N1}R^{N2}, C₁₋₆ alkyl, C₁₋₄ haloalkyl, phenyl, 5 to 6 membered heteroaryl, 3 to 7 membered monocyclic carbocyclyl and 4 to 7 membered monocyclic heterocyclyl;
R^{N1} and R^{N2} are each independently H, C₁₋₆ alkyl, 4 to 7 membered monocyclic heterocyclyl, 5 or 6 membered heteroaryl, or C₃₋₆ cycloalkyl, wherein the C₁₋₆ alkyl represented by R^{N1} and R^{N2} are each optionally substituted with C₁₋₄alkoxy or phenyl, and wherein the C₃₋₆ cycloalkyl, 4 to 7 membered monocyclic heterocyclyl, 5 or 6 membered heteroaryl represented by R^{N1} and R^{N2} are each optionally substituted with C₁₋₄alkyl;
each R^{N3} is independently H or C₁₋₆ alkyl; and
each R^{N4} is independently H or C₁₋₆ alkyl.

In one aspect, the present disclosure is a pharmaceutical composition comprising at least one compound described herein, or a pharmaceutically acceptable salt thereof, and at least one pharmaceutically acceptable carrier.

Another aspect of the present disclosure is a method of inhibiting TYK2 activity in a subject in need thereof comprising administering to the subject an effective amount of at least one compound described herein, or a pharmaceutically acceptable salt thereof, or a pharmaceutical composition described herein.

In some aspect, the present disclosure is a method of treating a disease or disorder responsive to inhibition of TYK2 in a subject comprising administering to the subject an effective amount of at least one compound described herein, or a pharmaceutically acceptable salt thereof, or a pharmaceutical composition described herein.

The present disclosure also includes the use of at least one compound described herein, or a pharmaceutically acceptable salt thereof, or a pharmaceutically composition described herein, for the manufacture of a medicament for inhibiting TYK2 activity. Also included is the use of at least one compound described herein, or a pharmaceutically acceptable salt thereof, or a pharmaceutically composition described herein, for the manufacture of a medicament for treating a disease or disorder responsive to inhibition of TYK2.

The disclosure also provides a compound described herein, or a pharmaceutically acceptable salt thereof, or a pharmaceutical composition described herein, for use in inhibiting TYK2 activity. Also provided is a compound described herein, or a pharmaceutically acceptable salt thereof, or a pharmaceutical composition described herein, for use in treating a disease or disorder responsive to inhibition of TYK2.

Other features or advantages will be apparent from the following detailed description of several embodiments, and also from the appended claims.

### DETAILED DESCRIPTION

The compounds or pharmaceutically acceptable salts thereof described herein demonstrate high potency against TYK2. In addition, the compounds or pharmaceutically acceptable salts thereof of the present disclosure have high selectivity for inhibiting TYK2 over other members of JAK family, such as JAK1 and JAK2.

### I. Definitions

Compounds are described using standard nomenclature. Unless defined otherwise, all technical and scientific terms used herein have the same meaning as is commonly understood by one of ordinary skill in the relevant art.

The terms “a” and “an” do not denote a limitation of quantity, but rather denote the presence of at least one of the referenced item. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of examples, or exemplary language (e.g., “such as”), is intended merely to better illustrate the invention and does not pose a limitation on the scope of the invention unless otherwise claimed.

As used herein, the term “alkyl” refers to a fully saturated branched or unbranched hydrocarbon moiety. In some embodiments, the alkyl comprises 1 to 20 carbon atoms, 1 to 10 carbon atoms, 1 to 8 carbon atoms, 1 to 6 carbon atoms, or 1 to 4 carbon atoms. In some embodiments, an alkyl comprises from 6 to 20 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, or n-hexyl. When indicated as being “optionally substituted”, the alkane radical or alkyl moiety may be unsubstituted or substituted with one or more substituents (generally, one to three substituents except in the case of halogen substituents such as perchloro or perfluoroalkyls).

As used herein, the term “alkoxy” refers to a fully saturated branched or unbranched alkyl moiety attached through an oxygen bridge (i.e. a —O—C₁₋₄ alkyl group wherein C₁₋₄ alkyl is as defined herein). Representative examples of alkoxy include, but are not limited to, methoxy, ethoxy, propoxy, 2-propoxy, butoxy, tert-butoxy and the like. Preferably, alkoxy groups have about 1-4 carbons, more preferably about 1-2 carbons.

As used herein, the number of carbon atoms in a group is specified herein by the prefix “Cₓ₋ₓₓ”, wherein x and xx are integers. For example, “C₁₋₄ alkyl” is an alkyl group which has from 1 to 4 carbon atoms

As used herein, the term “aryl” refers to a carbocyclic (all carbon) aromatic monocyclic or bicyclic ring system containing 6-10 carbon atoms. Examples of 6-10 membered aryl groups include phenyl and naphthyl. In some embodiments, the aryl is phenyl.

The term “cycloalkyl” refers to completely saturated monocyclic or bicyclic or spiro hydrocarbon groups of 3-7 carbon atoms, 3-6 carbon atoms, or 5-7 carbon atoms. In some embodiments, cycloalkyl is a 3- to 6-membered monocyclic cycloalkyl.

As used herein, the terms “carbocycle”, “carbocyclyl” and “carbocyclic ring” refer to saturated or partially unsaturated (i.e., non-aromatic) monocyclic or bicyclic hydrocarbon groups of, for example, 3-10, 3-8, 3-7, 3-5, 3-6, 4-6, 5-7 or 7-10 carbon atoms.

“Halogen” or “halo” may be fluoro, chloro, bromo or iodo.

As used herein, the term “haloalkyl” or “halo-substituted alkyl” or refers to an alkyl group as defined herein, wherein at least one of the hydrogen atoms is replaced by a halo atom. The haloalkyl group can be monohalo-alkyl, dihaloalkyl or polyhaloalkyl including perhaloalkyl. A monohaloalkyl can have one iodo, bromo, chloro or fluoro within the alkyl group. Dihaloalkyl and polyhaloalkyl groups can have two or more of the same halo atoms or a combination of different halo groups within the alkyl. Typically the polyhaloalkyl group contains up to 9, or 8, or 7, or 6, or 5, or 4, or 3, or 2 halo groups. Non-limiting examples of haloalkyl include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. A perhaloalkyl group refers to an alkyl group having all hydrogen atoms replaced with halo atoms.

As used herein, the term “heteroaryl” refers to an aromatic 5- to 6-membered monocyclic or an 8- to 10-membered bicyclic ring system, having 1 to 4 heteroatoms independently selected from O, N and S, and wherein N can be oxidized (e.g., N(O)) or quaternized, and S can be optionally oxidized to sulfoxide and sulfone. Examples of 5- to 6-membered monocyclic heteroaryls include, but are not limited to, pyrrolyl, furanyl, thiophenyl (or thienyl), imidazolyl, pyrazolyl, oxazolyl, isoxazolyl, thiazolyl, isothiazolyl, furazanyl, oxadiazolyl, thiadiazolyl, dithiazolyl, triazolyl, tetrazolyl, pyridinyl, pyrazinyl, pyrimidinyl, pyridazinyl, triazinyl, tetrazinyl, and the like. Examples of 8- to 10-membered bicyclic heteroaryls include, but are not limited to, imidazolthiazolyl, imidazopyridinyl, imidazo[1,2-a]pyridinyl, imidazo[2,1-b]thiazolyl, indazolyl, 2H-indazolyl, indolyl, isoindolyl, 2λ²-isoindolinyl, benzimidazolyl, benzofuranyl, benzothiophenyl, benzothiazolyl, quinolinyl, isoquinolinyl, quinazolinyl, purinyl, thienopyridinyl and thieno[3,2-b]pyridinyl.

As used herein, the term “heterocyclyl” refers to a saturated or unsaturated, monocyclic or bicyclic (e.g., fused, bridged or spiro ring systems) ring system which has from 3- to 14-ring members, or in particular 3- to 8-ring members, 3- to 7-ring members, 3- to 6-ring members or 5- to 7-ring members, 4- to 7-ring members or 4- to 6-ring members, at least one of which is a heteroatom, and up to 4 (e.g., 1, 2, 3, or 4) of which may be heteroatoms, wherein the heteroatoms are independently selected from O, S and N, and wherein C can be oxidized (e.g., C(O)), N can be oxidized (e.g., N(O)) or quaternized, and S can be optionally oxidized to sulfoxide and sulfone. The heterocyclyl group can be attached to the rest of a compound of the invention at a heteroatom or a carbon atom. The term azacyclic refers to a non-aromatic heterocyclyl, which has at least one nitrogen ring atom. The examples of azacyclic include, but are not limited to, azetidine, pyrrolidine, piperidine, piperazine, and morpholine. Fully saturated heterocyclyl groups include heterocycloalkyl groups. Examples of 3- to 7-membered monocyclic heterocyclyl include, but are not limited to, aziridinyl, oxiranyl, thirranyl, oxaziridinyl, oxazepanyl, azetidinyl, oxetanyl, thietanyl, pyrrolidinyl, tetrahydrofuranyl, thiolanyl, imidazolidinyl, pyrazolidinyl, oxazolidinyl, isoxazolidinyl, thiazolidinyl, isothiazolidinyl, dioxolanyl, dithiolanyl, oxathiolanyl, piperidinyl, tetrahydropyranyl, thianyl, piperazinyl, morpholinyl, thiomorpholinyl, dioxanyl, dithianyl, trioxanyl, trithianyl, azepanyl, oxepanyl, thiepanyl, dihydrofuranyl, imidazolinyl, and dihydropyranyl. In one embodiment, a heterocyclyl is a 5- to 7-membered monocyclic heterocyclyl (saturated or partially unsaturated). Examples include pyrrolidinyl, tetrahydrofuranyl, thiolanyl, imidazolidinyl, pyrazolidinyl, oxazolidinyl, oxazepanyl, isoxazolidinyl, thiazolidinyl, isothiazolidinyl, dioxolanyl, dithiolanyl, oxathiolanyl, piperidinyl, tetrahydropyranyl, thianyl, piperazinyl, morpholinyl, thiomorpholinyl, dioxanyl, dithianyl, trioxanyl, trithianyl, azepanyl, oxepanyl, thiepanyl, dihydrofuranyl, imidazolinyl, and dihydropyranyl.

The term “bicyclic heterocycle” refers to a bicyclic ring which is partially or fully saturated and contains 1 to 2 heteroatoms, independently selected from sulfur, oxygen and/or nitrogen.

The term “partially or fully saturated heterocycle” refers to a nonaromatic ring that is either partially or fully saturated and may exist as a single ring, bicyclic ring (including fused heterocyclic rings) or a spiro ring. Unless specified otherwise, the heterocyclic ring is generally a 3 to 7 membered ring containing 1 to 3 heteroatoms (preferably 1, 2 or 3 heteroatoms) independently selected from sulfur, oxygen and/or nitrogen.

As used herein “Hydroxyl” or “Hydroxy” refers to the group —OH.

The term “fused ring system”, as used herein, is a ring system that has two ring structures sharing two adjacent ring atoms. In one embodiment, a fused ring system have from 8 to 12 ring members.

The term “bridged ring system”, as used herein, is a ring system that has a carbocyclyl or heterocyclyl ring wherein two non-adjacent atoms of the ring are connected (bridged) by one or more (preferably from one to three) atoms selected from C, N, O, and S. In one embodiment, a bridged ring system have from 6 to 8 ring members.

The term “spiro ring system,” as used herein, is a ring system that has two ring structures having one ring atom in common. In one embodiment, spiro ring systems have from 5 to 8 ring members.

As used herein, the phrase “optionally substituted” is used interchangeably with the phrase “substituted or unsubstituted.” In general the term “optionally substituted” refers to the replacement of hydrogen radicals in a given structure with the radical of a specified substituent. Specific substituents are described in the definitions and in the description of compounds and examples thereof. Unless otherwise indicated, an optionally substituted group can have a substituent at each substitutable position of the group, and when more than one position in any given structure can be substituted with more than one substituent selected from a specified group, the substituent can be either the same or different at every position.

The term “oxo” (═O) refers to an oxygen atom connected to a carbon or sulfur atom by a double bond. Examples include carbonyl, sulfinyl, or sulfonyl groups (—C(O)—, —S(O)— or —S(O)₂—) such as, a ketone, aldehyde, or part of an acid, ester, amide, lactone, or lactam group and the like.

Unless specified otherwise, the term “compounds of the present disclosure” refers to compounds of formula (I), (II), (III), (IV), (V), (VI), (VII), (VII′), (VIIA), (VIIA′), (VIIB), (VIII), or (VIII′), as well as all stereoisomers (including diastereoisomers and enantiomers), rotamers, tautomers, isotopically labeled compounds (including deuterium substitutions). When a moiety is present that is capable of forming a salt, then salts are included as well, in particular pharmaceutically acceptable salts.

Compounds of the present disclosure may be synthesized by synthetic routes that include processes analogous to those well-known in the chemical arts, particularly in light of the description contained herein. The starting materials are generally available from commercial sources such as Sigma-Aldrich or are readily prepared using methods well known to those skilled in the art (e.g., prepared by methods generally described in Louis F. Fieser and Mary Fieser, Reagents for Organic Synthesis, v. 1-19, Wiley, New York (1967-1999 ed.), or Beilsteins Handbuch der organischen Chemie, 4, Aufl. ed. Springer-Verlag, Berlin, including supplements (also available via the Beilstein online database)). The protection of functional groups by protecting groups, the protecting groups themselves, and their cleavage reactions are described for example in standard reference works, such as J. F. W. McOmie, “Protective Groups in Organic Chemistry”, Plenum Press, London and New York 1973, in T. W. Greene and P. G. M. Wuts, “Protective Groups in Organic Synthesis”, Third edition, Wiley, New York 1999, in “The Peptides”; Volume 3 (editors: E. Gross and J. Meienhofer), Academic Press, London and New York 1981, in “Methoden der organischen Chemie” (Methods of Organic Chemistry), Houben Weyl, 4th edition, Volume 15/I, Georg Thieme Verlag, Stuttgart 1974, and in H.-D. Jakubke and H. Jeschkeit, “Aminosauren, Peptide, Proteine” (Amino acids, Peptides, Proteins), Verlag Chemie, Weinheim, Deerfield Beach, and Basel 1982. A characteristic of protecting groups is that they can be removed readily (i.e. without the occurrence of undesired secondary reactions) for example by solvolysis, reduction, photolysis or alternatively under physiological conditions (e.g. by enzymatic cleavage).

The compounds and intermediates described herein may be isolated and used as the compound per se. Alternatively, when a moiety is present that is capable of forming a salt, the compound or intermediate may be isolated and used as its corresponding salt. As used herein, the terms “salt” or “salts” refers to an acid addition or base addition salt of a compound of the disclosure. “Salts” include in particular “pharmaceutical acceptable salts”.

The term “pharmaceutically acceptable salts” refers to salts that retain the biological effectiveness and properties of the compounds of this disclosure and, which typically are not biologically or otherwise undesirable. In many cases, the compounds of the present disclosure are capable of forming acid and/or base salts by virtue of the presence of amino and/or carboxyl groups or groups similar thereto.

Salts of compounds of the present disclosure having at least one salt-forming group may be prepared in a manner known to those skilled in the art. For example, acid addition salts of compounds of the present disclosure are obtained in customary manner, e.g. by treating the compounds with an acid or a suitable anion exchange reagent. Salts can be converted into the free compounds in accordance with methods known to those skilled in the art. Acid addition salts can be converted, for example, by treatment with a suitable basic agent.

Pharmaceutically acceptable acid addition salts can be formed with inorganic acids and organic acids, e.g., acetate, aspartate, benzoate, besylate, bromide/hydrobromide, bicarbonate/carbonate, bisulfate/sulfate, camphorsulfornate, chloride/hydrochloride, chlortheophyllonate, citrate, ethandisulfonate, fumarate, gluceptate, gluconate, glucuronate, hippurate, hydroiodide/iodide, isethionate, lactate, lactobionate, laurylsulfate, malate, maleate, malonate, mandelate, mesylate, methylsulphate, naphthoate, napsylate, nicotinate, nitrate, octadecanoate, oleate, oxalate, palmitate, pamoate, phosphate/hydrogen phosphate/dihydrogen phosphate, polygalacturonate, propionate, stearate, succinate, sulfate, sulfosalicylate, tartrate, tosylate and trifluoroacetate salts.

Inorganic acids from which salts can be derived include, for example, hydrochloric acid, hydrobromic acid, sulfuric acid, nitric acid, phosphoric acid, and the like.

Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like. Pharmaceutically acceptable base addition salts can be formed with inorganic and organic bases.

Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

The salts can be synthesized by conventional chemical methods from a compound containing a basic or acidic moiety. Generally, such salts can be prepared by reacting free acid forms of these compounds with a stoichiometric amount of the appropriate base (such as Na, Ca, Mg, or K hydroxide, carbonate, bicarbonate or the like), or by reacting free base forms of these compounds with a stoichiometric amount of the appropriate acid. Such reactions are typically carried out in water or in an organic solvent, or in a mixture of the two. Generally, use of non-aqueous media like ether, ethyl acetate, ethanol, isopropanol, or acetonitrile is desirable, where practicable. Lists of additional suitable salts can be found, e.g., in “Remington's Pharmaceutical Sciences”, 20th ed., Mack Publishing Company, Easton, Pa., (1985); and in “Handbook of Pharmaceutical Salts: Properties, Selection, and Use” by Stahl and Wermuth (Wiley-VCH, Weinheim, Germany, 2002).

In some embodiments, the disclosure provides deuterated compounds in which any or more positions occupied by hydrogen can include enrichment by deuterium above the natural abundance of deuterium. For example, one or more hydrogen atoms are replaced with deuterium at an abundance that is at least 3340 times greater than the natural abundance of deuterium, which is 0.015% (i.e., at least 50.1% incorporation of deuterium), at least 3500 (52.5% deuterium incorporation at each designated deuterium atom), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation). In one embodiment, hydrogen is present at all positions at its natural abundance.

Isotopically-labeled compounds of formula (I) can generally be prepared by conventional techniques known to those skilled in the art or by processes analogous to those described in the accompanying Examples and Preparations using an appropriate isotopically-labeled reagents in place of the non-labeled reagent previously employed.

Pharmaceutically acceptable solvates in accordance with the disclosure include those wherein the solvent of crystallization may be isotopically substituted, e.g. D₂O, d₆-acetone, d₆-DMSO.

It will be recognized by those skilled in the art that the compounds of the present disclosure may contain chiral centers and as such may exist in different stereoisomeric forms. As used herein, the term “an optical isomer” or “a stereoisomer” refers to any of the various stereo isomeric configurations which may exist for a given compound of the present disclosure. It is understood that a substituent may be attached at a chiral center of a carbon atom. Therefore, the disclosure includes enantiomers, diastereomers or racemates of the compound.

Any resulting mixtures of isomers can be separated on the basis of the physicochemical differences of the constituents, into the pure or substantially pure geometric or optical isomers, diastereomers, racemates, for example, by chromatography and/or fractional crystallization.

For those compounds containing an asymmetric carbon atom, the compounds exist in individual optically active isomeric forms or as mixtures thereof, e.g. as racemic or diastereomeric mixtures. Diastereomeric mixtures can be separated into their individual diastereoisomers on the basis of their physical chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereoisomers and converting (e.g., hydrolyzing) the individual diastereoisomers to the corresponding pure enantiomers. Enantiomers can also be separated by use of a commercially available chiral HPLC column.

Certain of the compounds described herein contain one or more asymmetric centers or axes and may thus give rise to enantiomers, diastereomers, and other stereoisomeric forms that may be defined, in terms of absolute stereochemistry, as (R)- or (S)-. In accordance with the present disclosure any structure that does not designate the stereochemistry is to be understood as embracing all the various stereoisomers (e.g., diastereomers and enantiomers) in pure or substantially pure form, as well as mixtures thereof (such as a racemic mixture, or an enantiomerically enriched mixture). It is well known in the art how to prepare such optically active forms (for example, resolution of the racemic form by recrystallization techniques, synthesis from optically-active starting materials, by chiral synthesis, or chromatographic separation using a chiral stationary phase). In some embodiment, the compounds described herein are isolated stereoisomers wherein each of the compounds has one stereocenter and the stereoisomer is in the R configuration. In other embodiment, the compounds described herein are isolated stereoisomers wherein each of the compounds has one stereocenter and the stereoisomer is in the S configuration. In one embodiment, the compounds described herein are isolated stereoisomers wherein each of the compounds has two stereocenters and the stereoisomer is in the R R configuration. In one embodiment, the compounds described herein are isolated stereoisomers wherein each of the compounds has two stereocenters and the stereoisomer is in the R S configuration. In one embodiment, the compounds described herein are isolated stereoisomers stereoisomer wherein each of the compounds has two stereocenters and the stereoisomer is in the S R configuration. In one embodiment, the compounds described herein are isolated stereoisomers stereoisomer wherein each of the compounds has two stereocenters and the stereoisomer is in the S S configuration. In one embodiment, the compounds described herein each have one or two stereocenters and are racemic mixtures.

When a particular stereoisomer of a compound is depicted by name or structure, the stereochemical purity* of the compounds is at least 20%, 30%, 40%, 50%, 60%, 70%, 80%, 90%, 95%, 97%, 99%, 99.5% or 99.9%. “Stereochemical purity” means the weight percent of the desired stereoisomer relative to the combined weight of all stereoisomers.

When a disclosed compound is named or depicted by structure without indicating the stereochemistry, and the compound has one chiral center, it is to be understood that the name or structure encompasses one enantiomer of compound in pure or substantially pure form, as well as mixtures thereof (such as a racemic mixture of the compound and mixtures enriched in one enantiomer relative to its corresponding optical isomer).

It will be recognized by those skilled in the art that the compounds of the present disclosure may contain chiral centers and as such may exist in different stereoisomeric forms. As used herein, the term “an optical isomer” or “a stereoisomer” refers to any of the various stereo isomeric configurations which may exist for a given compound of the present disclosure. It is understood that a substituent may be attached at a chiral center of a carbon atom. Therefore, the disclosure includes enantiomers, diastereomers or racemates of the compound.

“Enantiomers” are a pair of stereoisomers that are non-superimposable mirror images of each other. A 1:1 mixture of a pair of enantiomers is a “racemic” mixture. The term is used to designate a racemic mixture where appropriate. When designating the stereochemistry for the compounds of the present disclosure, a single stereoisomer with known relative and absolute configuration of the two chiral centers is designated using the conventional RS system (e.g., (1S,2S)); a single stereoisomer with known relative configuration but unknown absolute configuration is designated with stars (e.g., (1R*,2R*)); and a racemate with two letters (e.g, (1RS,2RS) as a racemic mixture of (1R,2R) and (1S,2S); (1RS,2SR) as a racemic mixture of (1R,2S) and (1S,2R)).

“Diastereoisomers” are stereoisomers that have at least two asymmetric atoms, but which are not mirror-images of each other. The absolute stereochemistry is specified according to the Cahn-Ingold-Prelog R-S system. When a compound is a pure enantiomer the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. Alternatively, the resolved compounds can be defined by the respective retention times for the corresponding enantiomers/diastereomers via chiral HPLC.

Certain of the compounds described herein contain one or more asymmetric centers or axes and may thus give rise to enantiomers, diastereomers, and other stereoisomeric forms that may be defined, in terms of absolute stereochemistry, as (R)- or (S)-.

Unless specified otherwise, the compounds of the present disclosure are meant to include all such possible stereoisomers, including racemic mixtures, optically pure forms and intermediate mixtures. Optically active (R)- and (S)-stereoisomers may be prepared using chiral synthons or chiral reagents, or resolved using conventional techniques (e.g., separated on chiral SFC or IPLC chromatography columns, such as CHIRALPAK® and CHIRALCEL® available from DAICEL Corp. using the appropriate solvent or mixture of solvents to achieve good separation). If the compound contains a double bond, the substituent may be E or Z configuration. If the compound contains a disubstituted cycloalkyl, the cycloalkyl substituent may have a cis- or trans-configuration. All tautomeric forms are also intended to be included.

The disclosed compounds may exist in tautomeric forms and mixtures and separate individual tautomers are contemplated. All such forms are embraced within the scope of the disclosure. In addition, some compounds may exhibit polymorphism. The term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerizations. A specific example of a proton tautomer is the imidazole moiety where the proton may migrate between the two ring nitrogens. Valence tautomers include interconversions by reorganization of some of the bonding electrons.

### II. Compounds of the Disclosure

In a first aspect, the compound of the present disclosure is represented by Formula (I), or a pharmaceutically acceptable salt thereof, wherein the variables are as described above.

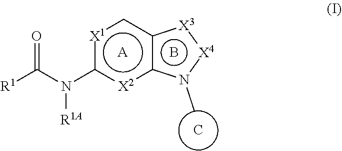

In a first embodiment, the compound of the present disclosure is represented by Formula (I), or a pharmaceutically acceptable salt thereof, wherein:
ring A is an aromatic or heteoaromatic ring fused with ring B that is a 5-membered heteroaromatic ring;
X¹ is N or CH;
X² is N or CR²;
X³ is N or CR³;
X⁴ is N or CR⁴;
ring C is phenyl or 5 to 6 membered heteroaryl, each of which is optionally substituted by one or more R^{C};
each R^{C} is independently halo, —CN, —NR^{N1}R^{N2}, —NR^{N3}—C(O)—R⁷, —NR^{N4}—SO₂—R⁷, —C(O)—R⁷, —SO₂—R⁷, —OR^{O1}, C₁₋₆ alkyl, 3 to 7 membered monocyclic carbocyclyl or 4 to 9 membered monocyclic or bicyclic heterocyclyl, wherein the C₁₋₆ alkyl, 3 to 7 membered monocyclic carbocyclyl and 4 to 9 membered monocyclic or bicyclic heterocyclyl represented by R^{C} are each optionally substituted with one or more R^{C1}, or two R^{C} taken together with intervening atoms form a 3 to 7 membered monocyclic carbocyclyl optionally substituted with one or more halo;
each R^{C1} is independently halo, oxo, —CN, —OR^{O1}, —NR^{N1}R^{N2}, —C(O)—R⁷, —C(O)—OR^{O3}, —SO₂—R⁷, C₁₋₆ alkyl, phenyl, 5 to 6 membered heteroaryl, or 4 to 7 membered monocyclic heterocyclyl, wherein the C₁₋₆ alkyl, phenyl, 5 to 6 membered heteroaryl and 4 to 7 membered monocyclic heterocyclyl represented by R^{C1} are each optionally substituted by one or more substituents independently selected from halo, oxo, —CN, —OR^{O1}, —NR^{N1}R^{N2}, C₁₋₆ alkyl, C₁₋₄ haloalkyl, phenyl, 5 to 6 membered heteroaryl, 3 to 7 membered monocyclic carbocyclyl and 4- to 8-membered monocyclic heterocyclyl;
R¹ is C₁₋₆ alkyl, phenyl, 5 to 6 membered heteroaryl, 3 to 7 membered monocyclic carbocyclyl or 4 to 7 membered monocyclic heterocyclyl, wherein the C₁₋₆ alkyl, phenyl, 5 to 6 membered heteroaryl, 3 to 7 membered monocyclic carbocyclyl and 4 to 7 membered monocyclic heterocyclyl represented by R¹ are each optionally substituted by one or more R⁸;
R^{1A} is H or C₁₋₃alkyl;
or R¹ and R^{1A} together with the atom from which they are attached form a 5 or 6 membered monocyclic heterocycle;
R² is H or halo;
R³ is H, —NR^{N1}R^{N2}, —CN, halo, —C(O)—R⁷, —C(O)—OR^{O3}, —SO₂—R⁷, —OR^{O4}, C₁₋₆ alkyl, phenyl, 5 to 6 membered heteroaryl, 3 to 7 membered monocyclic carbocyclyl, or 4 to 9 membered monocyclic or bicyclic heterocyclyl, wherein the C₁₋₆ alkyl, phenyl, 5 to 6 membered heteroaryl, 3 to 7 membered monocyclic carbocyclyl and 4 to 9 membered monocyclic or bicyclic heterocyclyl represented by R³ are each optionally substituted by one or more R⁹;
R⁴ is H or halo;
each R⁷ is independently C₁₋₆ alkyl, phenyl, 5 to 6 membered heteroaryl, 3 to 7 membered monocyclic carbocyclyl or 4 to 7 membered monocyclic heterocyclyl; wherein the C₁₋₆ alkyl, phenyl, 5 to 6 membered heteroaryl, 3 to 7 membered monocyclic carbocyclyl and 4 to 7 membered monocyclic heterocyclyl represented by R⁷ are each optionally substituted by one or more substituents independently selected from halo, oxo, —CN, —OR^{O1}, —NR^{1a}R^{1b}, C₁₋₆ alkyl, C₁₋₄ haloalkyl, 3 to 7 membered monocyclic carbocyclyl and 4 to 7 membered monocyclic heterocyclyl;
each R⁸ is independently halo, oxo, —CN, —OR^{O1}, C₁₋₆ alkyl, C₁₋₄ haloalkyl, phenyl, 5 to 6 membered heteroaryl, 3 to 7 membered monocyclic carbocyclyl or 4 to 7 membered monocyclic heterocyclyl;
each R⁹ is independently halo, oxo, —OR^{O1}, —NR^{N1}R^{N2}, —CN, —C(O)—OR^{O3}, —SO₂—R¹⁰, C₁₋₆ alkyl, phenyl, 5 to 6 membered heteroaryl, 3 to 7 membered monocyclic carbocyclyl or 4 to 7 membered monocyclic heterocyclyl, wherein the C₁₋₆ alkyl, phenyl, 5 to 6 membered heteroaryl, 3 to 7 membered monocyclic carbocyclyl and 4 to 7 membered monocyclic heterocyclyl represented by R⁹ are each optionally substituted by one or more substituents independently selected from halo, oxo, —CN, —OR^{O1}, —NR^{N1}R^{N2}, C₁₋₆ alkyl, C₁₋₄ haloalkyl, phenyl, 5 to 6 membered heteroaryl, 3 to 7 membered monocyclic carbocyclyl and 4 to 7 membered monocyclic heterocyclyl;
each R^{O1} is independently H, C₁₋₆ alkyl, phenyl, 5 to 6 membered heteroaryl, 3 to 7 membered monocyclic or bicyclic carbocyclyl, or 4 to 7 membered monocyclic or bicyclic heterocyclyl, wherein the C₁₋₆ alkyl, phenyl, 5 to 6 membered heteroaryl, 3 to 7 membered monocyclic or bicyclic carbocyclyl, and 4 to 7 membered monocyclic or bicyclic heterocyclyl represented by R^{O1} are each optionally substituted by one or more R^{O2};
each R^{O2} is independently halo, OH, —CN, C₁₋₄ alkoxy, C₁₋₄ alkyl, phenyl, 5 to 6 membered heteroaryl, 3 to 7 membered monocyclic carbocylyl or 4 to 7 membered monocyclic or bicyclic heterocyclyl, wherein the C₁₋₄ alkyl, phenyl, 5 to 6 membered heteroaryl, 3 to 7 membered monocyclic carbocylyl and 4 to 7 membered monocyclic or bicyclic heterocyclyl are each optionally substituted with one or more halo, C₁₋₆ alkyl or —O—C₁₋₆alkyl;
each R^{O3} is independently H, C₁₋₆ alkyl, C₁₋₄ haloalkyl, phenyl, 5 to 6 membered heteroaryl, 3 to 7 membered monocyclic carbocyclyl or 4 to 7 membered monocyclic heterocyclyl, wherein the C₁₋₆ alkyl, phenyl, 5 to 6 membered heteroaryl, 3 to 7 membered monocyclic carbocyclyl and 4 to 7 membered monocyclic heterocyclyl represented by R^{O3} are each optionally substituted by one or more R^{O2};
R^{O4} is H, C₁₋₆ alkyl, phenyl, 5 to 6 membered heteroaryl, 3 to 7 membered monocyclic carbocyclyl or 4 to 7 membered monocyclic heterocyclyl, wherein the C₁₋₆ alkyl, phenyl, 5 to 6 membered heteroaryl, 3 to 7 membered monocyclic carbocyclyl and 4 to 7 membered monocyclic heterocyclyl represented by R^{O4} are each optionally substituted by one or more substituents independently selected from halo, oxo, —CN, —OR^{O1}, —NR^{N1}R^{N2}, C₁₋₆ alkyl, C₁₋₄ haloalkyl, phenyl, 5 to 6 membered heteroaryl, 3 to 7 membered monocyclic carbocyclyl and 4 to 7 membered monocyclic heterocyclyl;
R^{N1} and R^{N2} are each independently H, C₁₋₆ alkyl or C₃₋₆ cycloalkyl, wherein the C₁₋₆ alkyl represented by R^{N1} and R^{N2} are each optionally substituted with C₁₋₄alkoxy;
each R^{N3} is independently H or C₁₋₆ alkyl; and
each R^{N4} is independently H or C₁₋₆ alkyl.

In a second embodiment, for compounds of formula (I) or pharmaceutically acceptable salts thereof, R^{1A} is H or —CH₃; and the remaining variables are as described in the first aspect or the first embodiment.

In a third embodiment, the compound is represented by formula (II), (III), (IV), (V) or (VI):

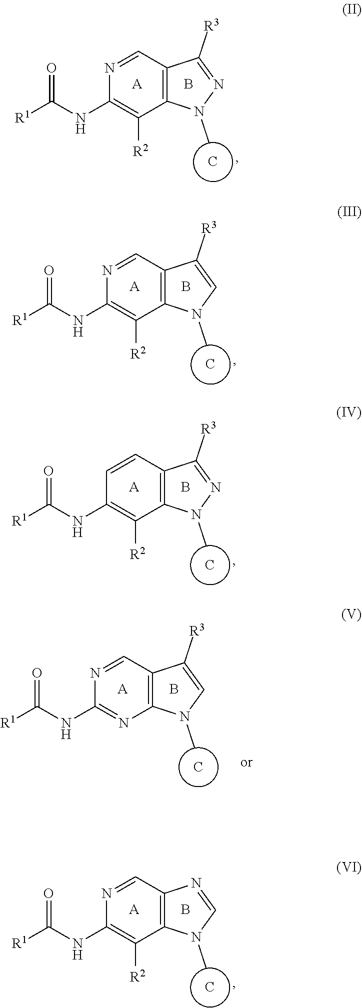

or a pharmaceutically acceptable salt thereof, and the variables are as described in the first aspect or the first embodiment.

In a fourth embodiment, for compounds of formula (I), (II), (III), (IV), (V) or (VI), or pharmaceutically acceptable salts thereof, R² is H or F; and the remaining variables are as described in the first aspect or the first, second or third embodiment.

In a fifth embodiment, for compounds of formula (I), (II), (III), (IV), (V) or (VI), or pharmaceutically acceptable salts thereof, R² is H; and the remaining variables are as described in the first aspect or the first, second or third embodiment.

In a sixth embodiment, for compounds of formula (I), (II), (III), (IV), (V) or (VI), or pharmaceutically acceptable salts thereof, ring C is phenyl or 5 to 6 membered heteroaryl, each of which is optionally substituted by one to three R^{C}; and the remaining variables are as described in the first aspect or the first, second, third, fourth or fifth embodiment. In some embodiments, ring C is not a pyridinyl group. In some embodiments, ring C is not a phenyl group. In an alternative sixth embodiment, for compounds of formula (I), (II), (III), (IV), (V) or (VI), or pharmaceutically acceptable salts thereof, ring C is phenyl, 5 or 6 membered monocyclic heterocyclyl, or 5 to 6 membered heteroaryl, each of which is optionally substituted by one to three R^{C}; and the remaining variables are as described in the first aspect or the first, second, third, fourth or fifth embodiment.

In a seventh embodiment, for compounds of formula (I), (II), (III), (IV), (V) or (VI), or pharmaceutically acceptable salts thereof, ring C is selected from imidazolyl, oxadiazolyl, oxazolyl, phenyl, pyrazinyl, pyridinyl, pyrimidinyl, thiadiazolyl, thiazolyl and triazinyl, each of which is optionally substituted by one or three R^{C}; and the remaining variables are as described in the first aspect or the first, second, third, fourth or fifth embodiment. In some embodiments, ring C is selected from imidazolyl, oxadiazolyl, oxazolyl, pyrazinyl, pyrimidinyl, thiadiazolyl, thiazolyl and triazinyl, each of which is optionally substituted by one or three R^{C}. In some embodiments, ring C is selected from pyrazinyl, pyrimidinyl and thiazolyl, each of which is optionally substituted by one or three R^{C}. In an alternative seventh embodiment, for compounds of formula (I), (II), (III), (IV), (V) or (VI), or pharmaceutically acceptable salts thereof, ring C is selected from pyridinonyl, pyridazinonyl, pyrazinonyl, imidazolyl, oxadiazolyl, oxazolyl, phenyl, pyrazinyl, pyridinyl, pyrimidinyl, thiadiazolyl, thiazolyl and triazinyl, each of which is optionally substituted by one or three R^{C}; and the remaining variables are as described in the first aspect or the first, second, third, fourth or fifth embodiment.

In an eighth embodiment, for compounds of formula (I), (II), (III), (IV), (V) or (VI), or pharmaceutically acceptable salts thereof, ring C is selected from:

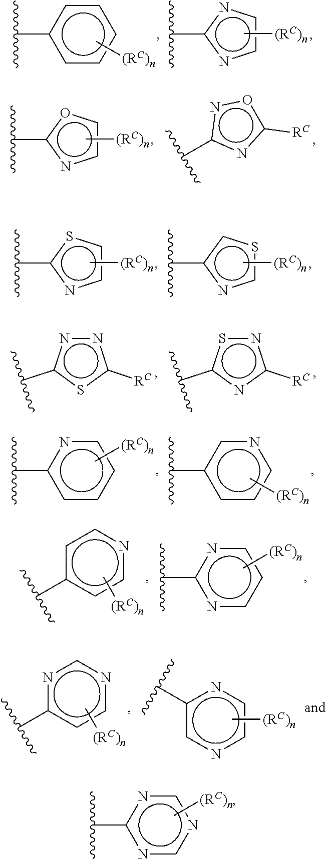

wherein

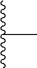

represents a bond to ring B, and n is 0, 1, 2, or 3; and the remaining variables are as described in the first aspect or the first, second, third, fourth or fifth embodiment. In some embodiments, ring C is selected from:

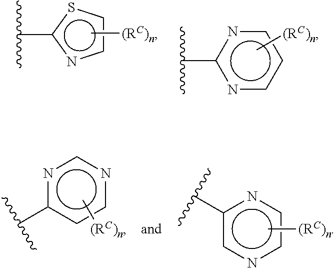

In an alternative eighth embodiment, for compounds of formula (I), (II), (III), (IV), (V) or (VI), or pharmaceutically acceptable salts thereof, ring C is selected from:

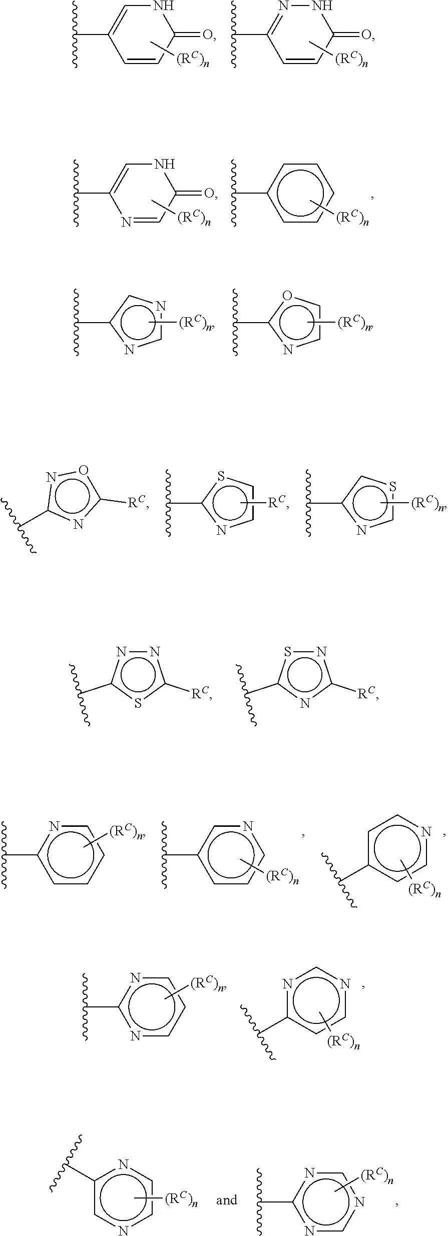

and wherein

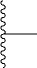

represents a bond to ring B, and n is 0, 1, 2, or 3; and the remaining variables are as described in the first aspect or the first, second, third, fourth or fifth embodiment.

In a ninth embodiment, for compounds of formula (I), (II), (III), (IV), (V) or (VI), or pharmaceutically acceptable salts thereof, ring C is selected from:

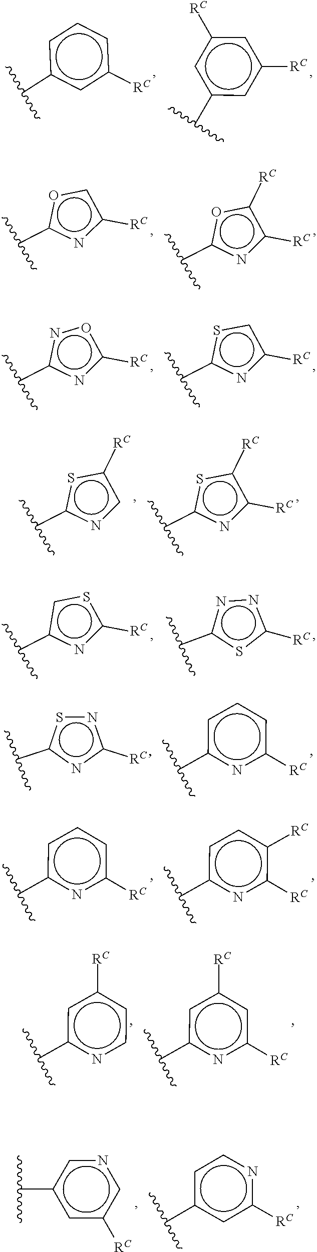

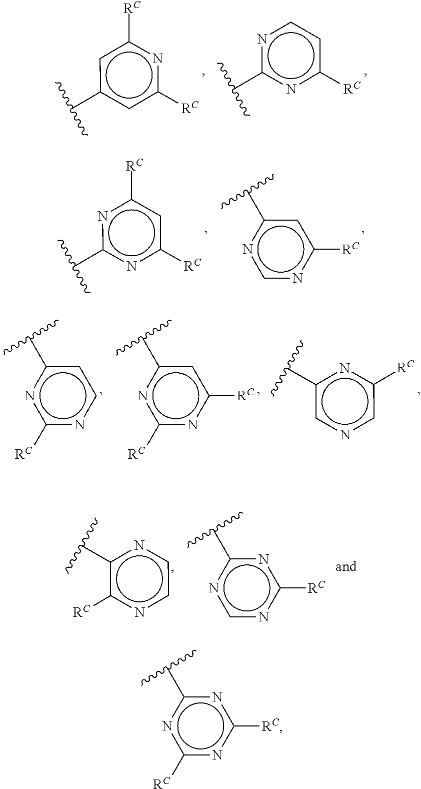

wherein

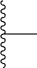

represents a bond to ring B, and two R^{C} groups in ring C may be the same or different; and the remaining variables are as described in the first aspect or the first, second, third, fourth or fifth embodiment. In some embodiments, ring C is selected from:

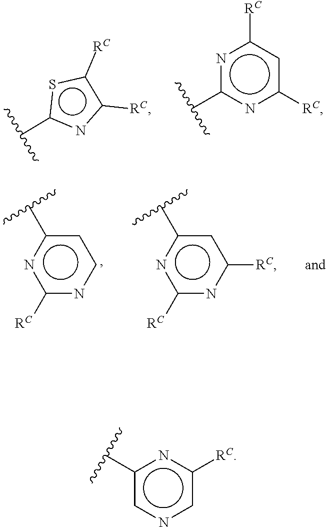

In some embodiments, ring C is

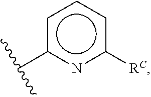

wherein R^{C} is C₁₋₃haloalkyl, more specifically C₂haloalkyl, even more specifically, —CF₂CH₃. In an alternative ninth embodiment, for compounds of formula (I), (II), (III), (IV), (V) or (VI), or pharmaceutically acceptable salts thereof, ring C is selected from:

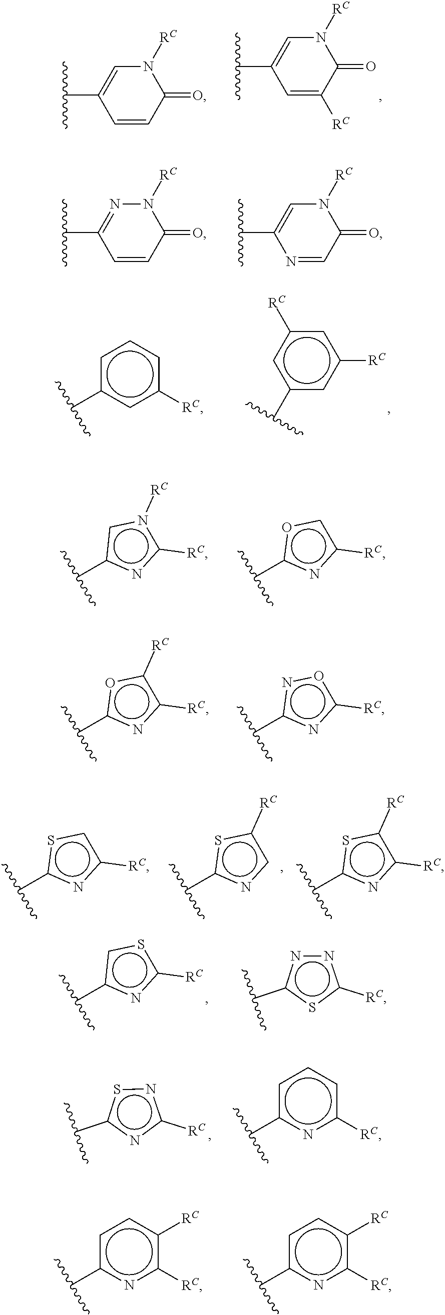

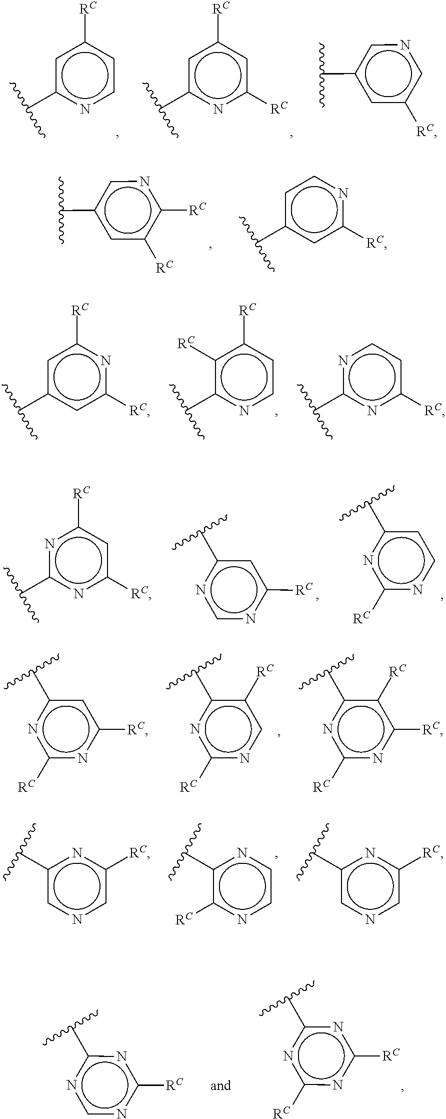

and wherein

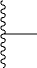

represents a bond to ring B, and two R^{C} groups in ring C may be the same or different; and the remaining variables are as described in the first aspect or the first, second, third, fourth or fifth embodiment.

In a tenth embodiment, for compounds of formula (I), (II), (III), (IV), (V) or (VI), or pharmaceutically acceptable salts thereof, each R^{C} is independently halo, —NR^{N1}R^{N2}, —NR^{N3}—C(O)—R⁷, —NR^{N4}—SO₂—R⁷, —C(O)—R⁷, —OR^{O1}, C₁₋₆ alkyl, C₃₋₆ cycloalkyl, or 4 to 8 membered monocyclic or bicyclic heterocyclyl, wherein the C₁₋₆ alkyl, C₃₋₆ cycloalkyl and 4 to 8 membered monocyclic or bicyclic heterocyclyl represented by R^{C} are each optionally substituted with one to three R^{C1}; and the remaining variables are as described in the first aspect or the first, second, third, fourth, fifth, sixth, seventh, eighth or ninth embodiment or any alternative embodiments described therein. In some embodiments, at least one R^{C} is C₁₋₃ haloalkyl. In some embodiments, at least one R^{C} is C₂haloalkyl. In some embodiments, at least one R^{C} is —CF₂CH₃. In an alternative tenth embodiment, for compounds of formula (I), (II), (III), (IV), (V) or (VI), or pharmaceutically acceptable salts thereof, each R^{C} is independently halo, —NR^{N1}R^{N2}, —NR^{N3}—C(O)—R⁷, —NR^{N4}—SO₂—R⁷, —C(O)—R⁷, —OR^{O1}, C₁₋₆ alkyl, alkenyl, C₃₋₆ cycloalkyl, phenyl, 5 to 12 membered monocyclic or bicyclic heteroaryl, or 4 to 8 membered monocyclic or bicyclic heterocyclyl, or two R^{C} taken together with intervening atoms form a 3 to 7 membered monocyclic carbocyclyl optionally substituted with one or two halo; wherein the C₁₋₆ alkyl, C₃₋₆ cycloalkyl, 5 to 12 membered monocyclic or bicyclic heteroaryl, and 4 to 8 membered monocyclic or bicyclic heterocyclyl represented by R^{C} are each optionally substituted with one to three R^{C1}; and the remaining variables are as described in the first aspect or the first, second, third, fourth, fifth, sixth, seventh, eighth or ninth embodiment or any alternative embodiments described therein.

In an eleventh embodiment, for compounds of formula (I), (II), (III), (IV), (V) or (VI), or pharmaceutically acceptable salts thereof, the C₃₋₆ cycloalkyl represented by R^{C} is selected from cyclobutane, cyclopentane, cyclopropane and cyclohexane, and the 4 to 8 membered monocyclic or bicyclic heterocyclyl represented by R^{C} is selected from azetidinyl, 2,6-diazaspiro[3.3]heptanyl, isothiazolidinyl, isothiazolidinedioxide, morpholinyl, oxabicycloheptanyl, oxetanyl, piperidinyl, piperizinyl, pyrrolidinyl, pyrrolidinonyl, tetrahydrofuranyl, tetrahydro-2H-pyranyl, 2-oxa-6-azaspiro[3.4]octanyl and 7-oxabicyclo[2.2.1]heptanyl, wherein each of the C₃₋₆ cycloalkyl and 4 to 8 membered monocyclic or bicyclic heterocyclyl is optionally substituted with one to three R^{C1}; and the remaining variables are as described in the first aspect or the first, second, third, fourth, fifth, sixth, seventh, eighth or ninth embodiment or any alternative embodiments described therein or any alternative embodiments described therein. In an alternative eleventh embodiment, for compounds of formula (I), (II), (III), (IV), (V) or (VI), or pharmaceutically acceptable salts thereof, wherein the C₃₋₆ cycloalkyl represented by R^{C} is selected from cyclobutyl, cyclopentyl, cyclopropyl and cyclohexyl, the 5 to 12 membered monocyclic or bicyclic heteroaryl represented by R^{C} is furanyl, pyrazoyl, imidazoyl, triazoyl, isoxazole, pyridinyl, pyrimidinyl, isoindolinyl, 3H-imidazo[4,5-b]pyridinyl, 1H-benzo[d][1,2,3]triazolyl, and the 4 to 8 membered monocyclic or bicyclic heterocyclyl represented by R^{C} is selected from azetidinyl, 2,6-diazaspiro[3.3]heptanyl, isothiazolidinyl, isothiazolidinedioxide, morpholinyl, oxabicycloheptanyl, oxetanyl, piperidinyl, piperizinyl, pyrrolidinyl, pyrrolidinonyl, tetrahydrofuranyl, pyridin-2(1H)-oyl, tetrahydro-2H-pyranyl, 2-oxabicyclo[2.1.1]hexanyl, 2-oxa-6-azaspiro[3.4]octanyl and 7-oxabicyclo[2.2.1]heptanyl, wherein each of the C₃₋₆ cycloalkyl, 5 to 12 membered monocyclic or bicyclic heteroaryl, and 4 to 8 membered monocyclic or bicyclic heterocyclyl is optionally substituted with one to three R^{C1}, or two R^{C} taken together with intervening atoms form cyclopentyl substituted with one or two halo; and the remaining variables are as described in the first aspect or the first, second, third, fourth, fifth, sixth, seventh, eighth or ninth embodiment or any alternative embodiments described therein.

In a twelfth embodiment, for compounds of formula (I), (II), (III), (IV), (V) or (VI), or pharmaceutically acceptable salts thereof, the C₃₋₆ cycloalkyl and the 4 to 8 membered monocyclic or bicyclic heterocyclyl represented by R^{C} is represented by the following formula:

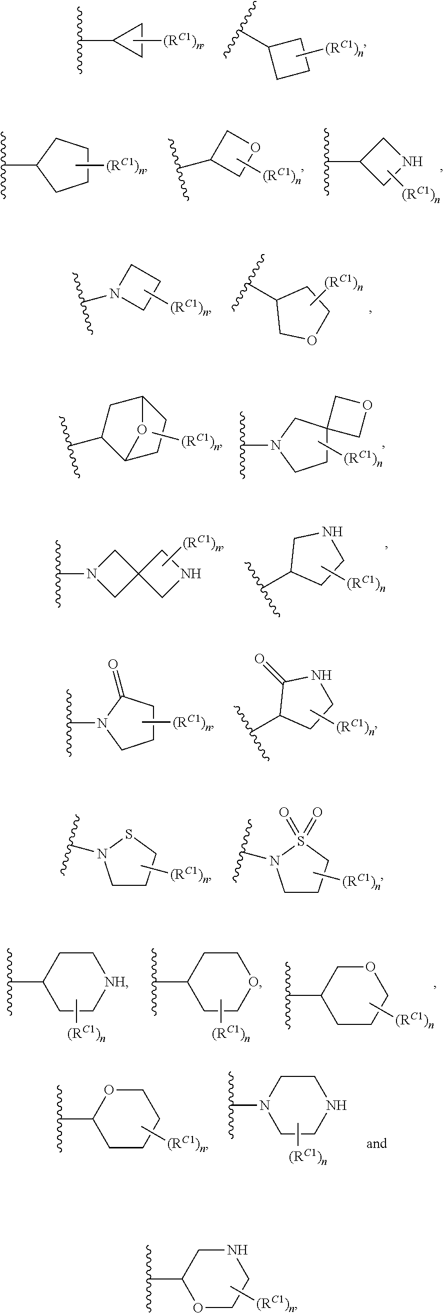

wherein

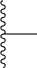

represents a bond to ring C, and n is 0, 1, 2 or 3; and the remaining variables are as described in the first aspect or the first, second, third, fourth, fifth, sixth, seventh, eighth or ninth embodiment or any alternative embodiments described therein. In an alternative twelfth embodiment, for compounds of formula (I), (II), (III), (IV), (V) or (VI), or pharmaceutically acceptable salts thereof, the C₃₋₆ cycloalkyl and the 4 to 8 membered monocyclic or bicyclic heterocyclyl represented by R^{C} is represented by the following formula:

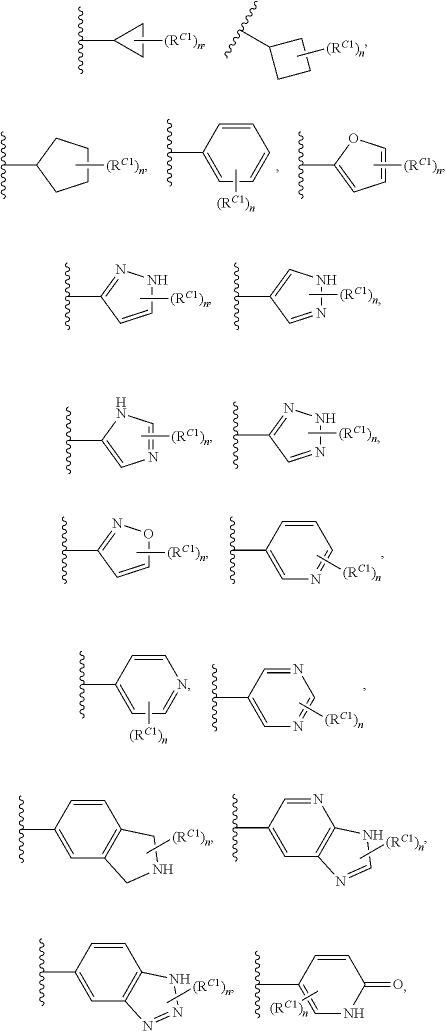

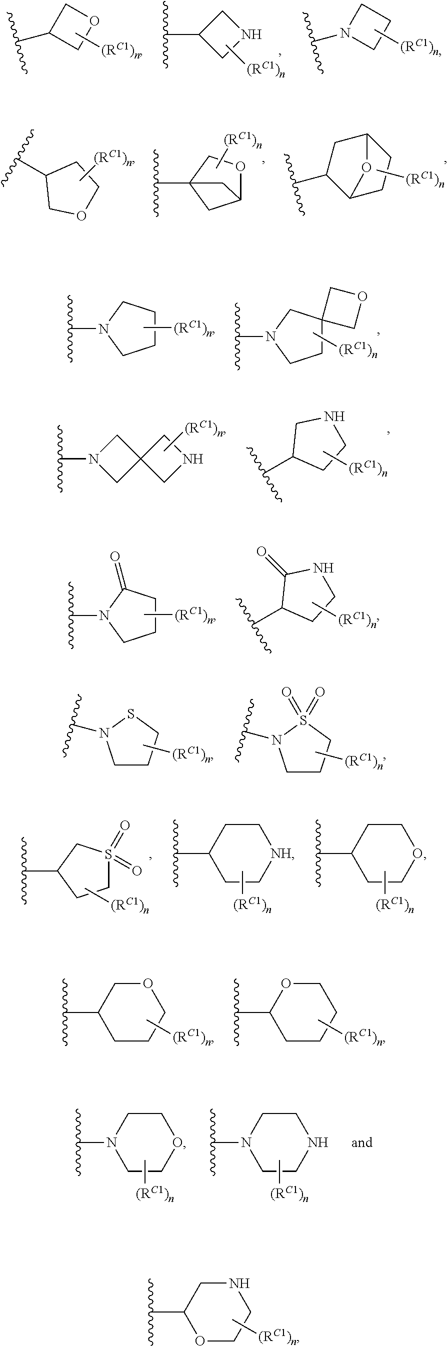

wherein

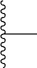

represents a bond to ring C, and n is 0, 1, 2 or 3; and the remaining variables are as described in the first aspect or the first, second, third, fourth, fifth, sixth, seventh, eighth or ninth embodiment or any alternative embodiments described therein.

In a thirteenth embodiment, for compounds of formula (I), (II), (III), (IV), (V) or (VI), or pharmaceutically acceptable salts thereof, the C₃₋₆ cycloalkyl and the 4 to 8 membered monocyclic or bicyclic heterocyclyl represented by R^{C} is independently selected from:

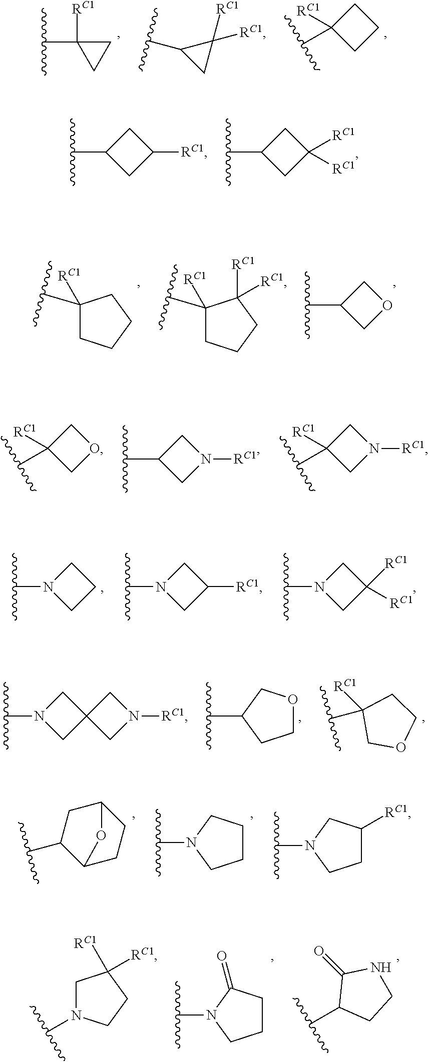

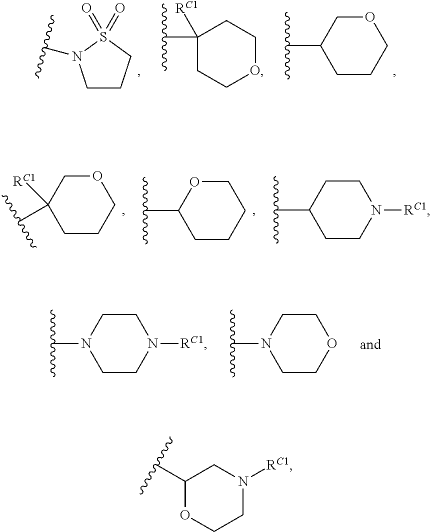

wherein

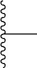

represents a bond ring C, and two R^{C1} groups may be the same or different; and the remaining variables are as described in the first aspect or the first, second, third, fourth, fifth, sixth, seventh, eighth or ninth embodiment or any alternative embodiments described therein. In an alternative thirteenth embodiment, for compounds of formula (I), (II), (III), (IV), (V) or (VI), or pharmaceutically acceptable salts thereof, the C₃₋₆ cycloalkyl and the 4 to 8 membered monocyclic or bicyclic heterocyclyl represented by R^{C} is independently selected from:

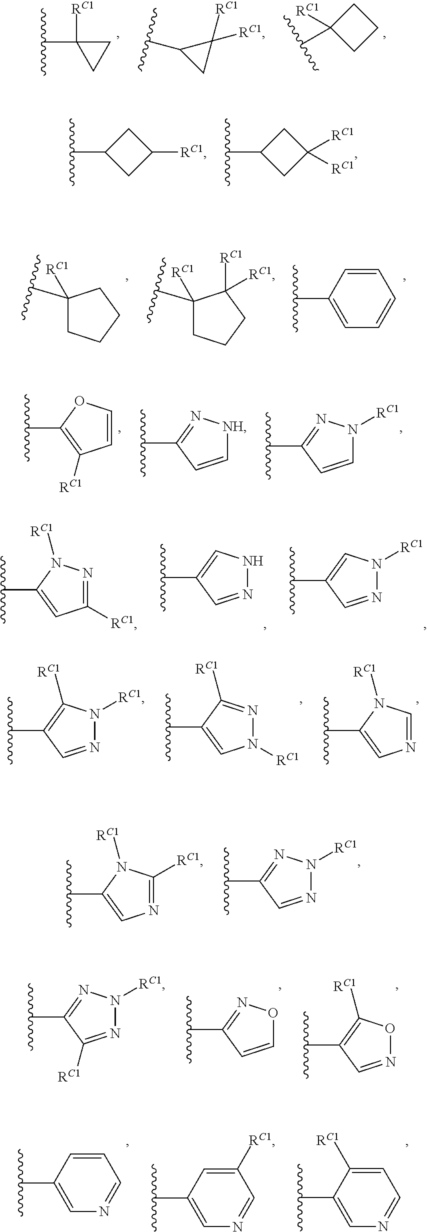

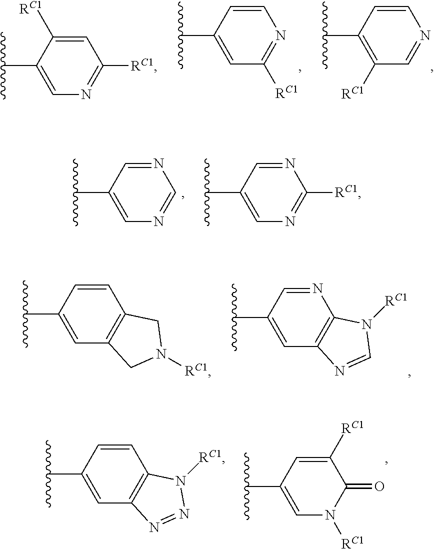

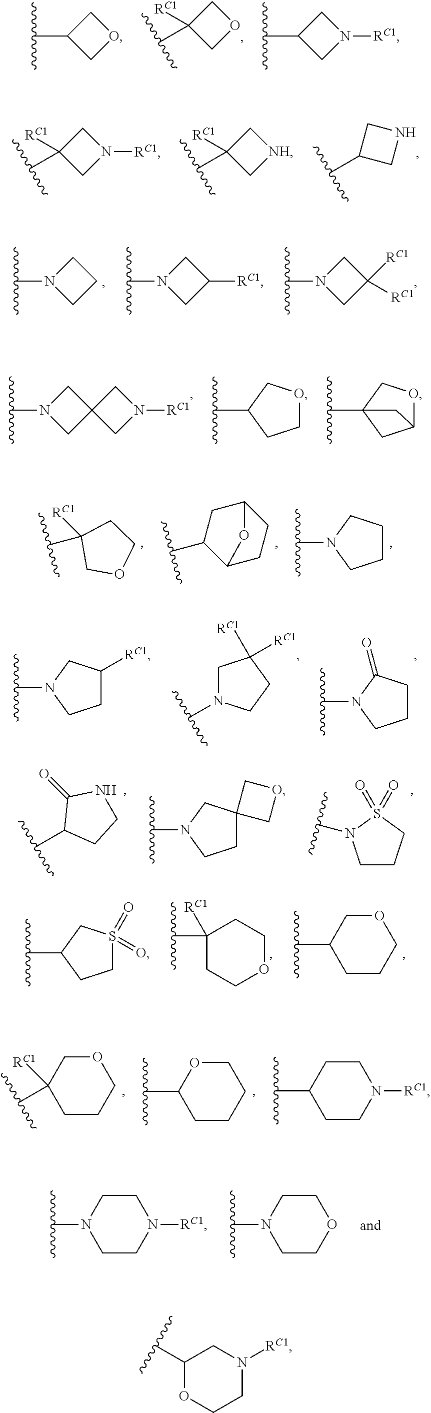

wherein

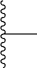

represents a bond ring C, and two R^{C1} groups may be the same or different; and the remaining variables are as described in the first aspect or the first, second, third, fourth, fifth, sixth, seventh, eighth or ninth embodiment or any alternative embodiments described therein.

In a fourteenth embodiment, for compounds of formula (I), (II), (III), (IV), (V) or (VI), or pharmaceutically acceptable salts thereof, each R^{C1} is independently halo, —CN, —OR^{O1}, —NR^{N1}R^{N2}, —C(O)—R⁷, —C(O)—OR^{O3}, —SO₂—R⁷, C₁₋₆ alkyl or 4 to 6 membered monocyclic heterocyclyl, wherein the C₁₋₆ alkyl represented by R^{C1} is optionally substituted by one to three substituents independently selected from halo and —OR^{a1}; and R^{a1} is H, C₁₋₄ alkyl or 4 to 6 membered heterocyclyl; and the remaining variables are as described in the first aspect or the first, second, third, fourth, fifth, sixth, seventh, eighth, ninth, tenth, eleventh, twelfth or thirteenth embodiment or any alternative embodiments described therein. In an alternative fourteenth embodiment, for compounds of formula (I), (II), (III), (IV), (V) or (VI), or pharmaceutically acceptable salts thereof, each R^{C1} is independently halo, —CN, —OR^{O1}, —NR^{N1}R^{N2}, —C(O)—R⁷, —C(O)—OR^{O3}, —SO₂—R⁷, C₁₋₆ alkyl, C₃₋₆cycloalkyl, or 4 to 6 membered monocyclic heterocyclyl, wherein the C₁₋₆ alkyl represented by R^{C1} is optionally substituted by one to three substituents independently selected from halo and —OR^{a1}; and R^{a1} is H, C₁₋₄ alkyl or 4 to 6 membered heterocyclyl; and the remaining variables are as described in the first aspect or the first, second, third, fourth, fifth, sixth, seventh, eighth, ninth, tenth, eleventh, twelfth or thirteenth embodiment or any alternative embodiments described therein.

In a fifteenth embodiment, for compounds of formula (I), (II), (III), (IV), (V) or (VI), or pharmaceutically acceptable salts thereof, each R^{C1} is independently selected from F, —CN, OH, —OCH₃, —OCHF₂, —NH₂, —N(CH₃)₂, —CH₃, —CH₂F, —CHF₂, —CF₃, —CH₂—OH, —CH₂—CH₃, —CH(CH₃)₂, —C(O)—CH₃, —C(O)—OC(CH₃)₃, —SO₂—CH₃,

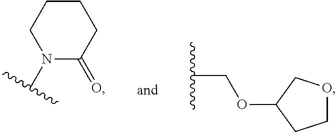 [SMILES: *N1CCCCC1=O ; "and" ; *COC1CCOC1]

wherein

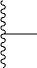

represents a bond to R^{C}; and the remaining variables are as described in the first aspect or the first, second, third, fourth, fifth, sixth, seventh, eighth, ninth, tenth, eleventh, twelfth or thirteenth embodiment. In an alternative fifteenth embodiment, for compounds of formula (I), (II), (III), (IV), (V) or (VI), or pharmaceutically acceptable salts thereof, each R^{C1} is independently selected from F, Cl, —CN, OH, —OCH₃, —OCHF₂, —NH₂, —N(CH₃)₂, —CH₃, —CH₂F, —CHF₂, —CF₃, —CH₂—OH, —CH₂—CH₃, —CH(CH₃)₂, —CH₂—O—CH₃, —CH₂—CH₂—OCH₃, —C(O)—CH₃, —C(O)—OC(CH₃)₃, —SO₂—CH₃, cyclopropyl,

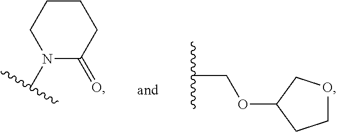 [SMILES: *N1CCCCC1=O ; "and" ; *COC1CCOC1]

wherein

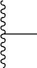

represents a bond to R^{C}; and the remaining variables are as described in the first aspect or the first, second, third, fourth, fifth, sixth, seventh, eighth, ninth, tenth, eleventh, twelfth or thirteenth embodiment or any alternative embodiments described therein.

In a sixteenth embodiment, for compounds of formula (I), (II), (III), (IV), (V) or (VI), or pharmaceutically acceptable salts thereof, each R^{C} is independently selected from Cl, —CH₃, —CH₂F, —CHF₂, —CF₃, —CH₂—CH₃, —CH₂—CH₂F, —CH₂—CHF₂, —CH₂—CF₃, —CHF—CH₂F, —CH₂—CHF₂, —CH₂—CF₃, —C(CH₃)₃, —CF₂CH₃, —CHF—CH₃, —CH(CH₃)₂, —CF(CH₃)₂, —(CH₃)₂—CH₂F, —CF(CH₃)₂, —CH(CH₃)—CHF₂, —CH(CH₃)—CF₃, —(CH₃)₂—CF₃, —CH₂—CH₂—CN, —CH₂—C(CH₃)₂—CN, —C(CH₃)₂—OH, —CH(CH₃)—OCH₃, —C(CH₃)₂—OCH₃, —CH(OH)—CH₃, —OCH₃, —O—CHF₂, —C(CH₃)(OCH₃)—CH₂—OCH₃, —O—CF₃, —CH(OCH₃)-cyclopropyl, —O—CH₂CH₃, —O—CH₂CHF₂, —O—CH(CH₃)₂, —O—CH(CF₃)₂, —O—CH₂—OCH₃, —O—CH(CH₃)—OCH₃, —O—CH(CH₃)—CH₂—OCH₃, —O(CH₃)₂—OCH₃, —O—CH₂—CH₂—O—CH₃, —O—CH₂—CH(CH₃)—OCH₃, —CF(CH₃)—CH₂—OCH₃, —CH(CF₃)—NH₂, —CH(OCH₃)—C(CH₃)₃, —NH—C(O)—CH₃, —NH—SO₂—CH₃, —N(CH₃)₂, —NHCH(CH₃)₂, —CHF-cyclopropyl, —CF(CH₃)-cyclopropyl, —NH-cyclohexyl, —N(CH₃)—CH₂—CH₂—OCH₃, —CH(NH₂)—CF₃,

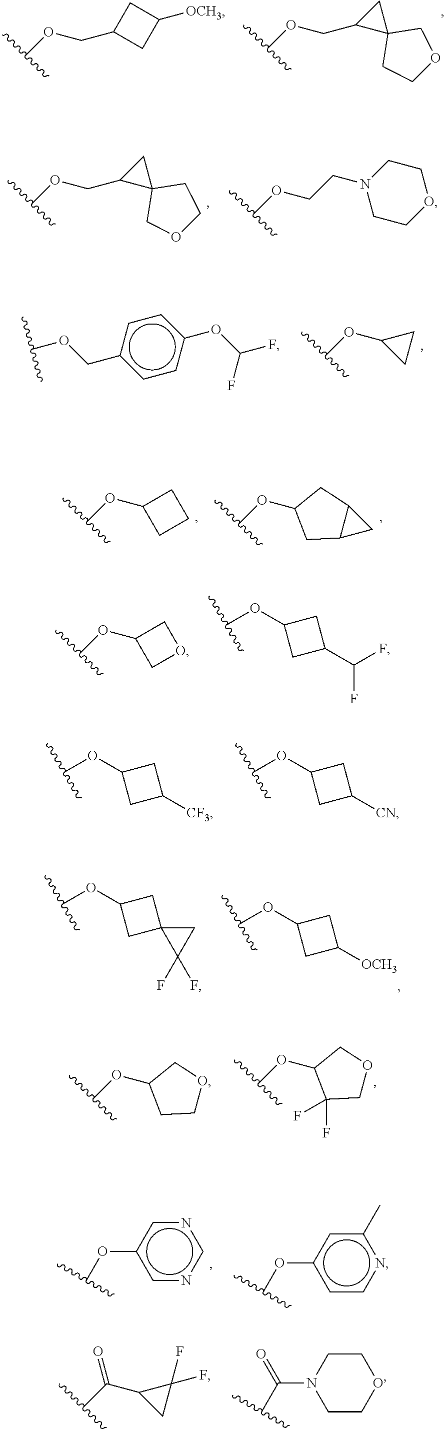

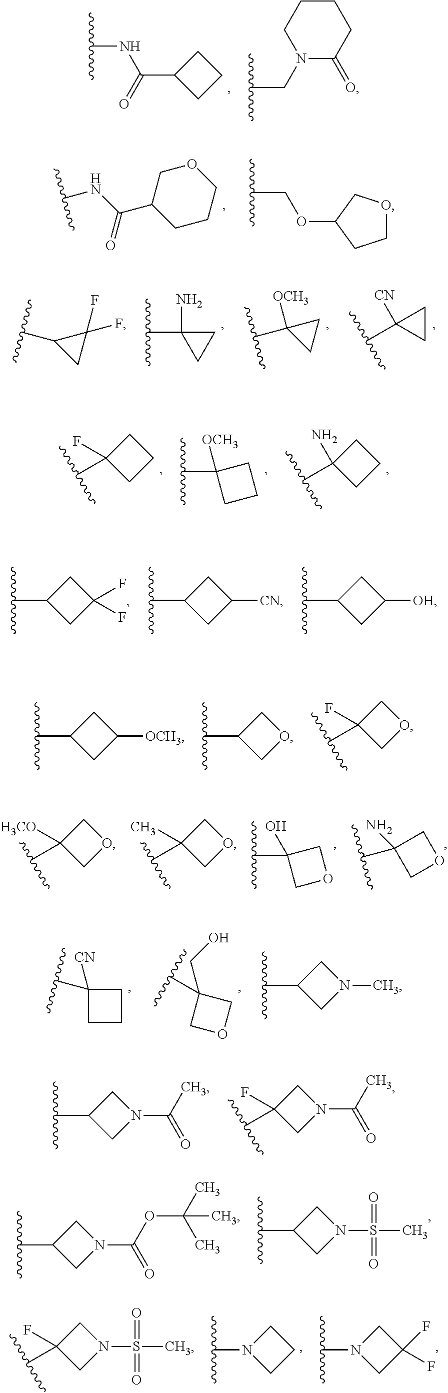

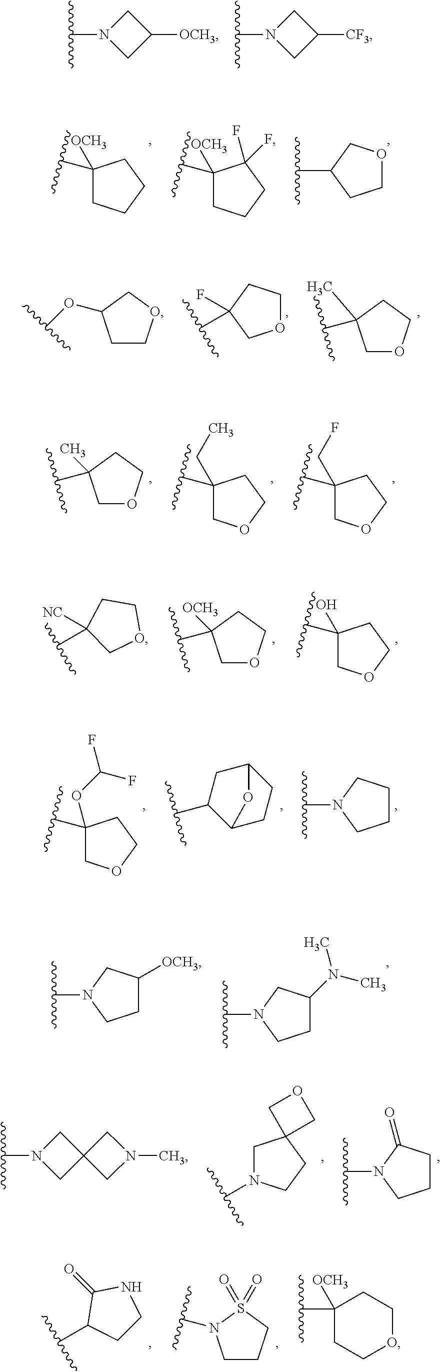

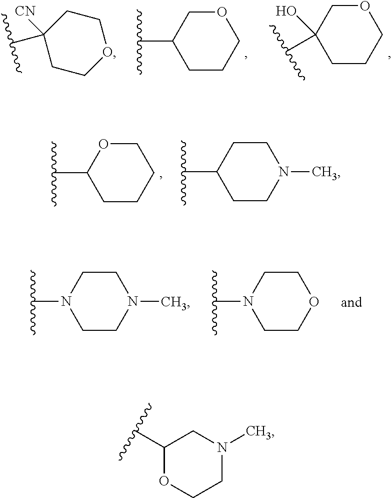

wherein

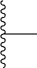

represents a bond to ring C; and the remaining variables are as described in the first aspect or the first, second, third, fourth, fifth, sixth, seventh, eighth or ninth embodiment or any alternative embodiments described therein. In one embodiment, R^{C} is —CF₂CH₃. In an alternative sixteenth embodiment, for compounds of formula (I), (II), (III), (IV), (V) or (VI), or pharmaceutically acceptable salts thereof, each R^{C} is independently selected from —F, —Cl, —CH₃, —CH₂F, —CHF₂, —CF₃, —CH₂—CH₃, —CH₂—CH₂F, —CH₂—CHF₂, —CH₂—CF₃, —CHF—CH₂F, —CH₂—CHF₂, —CH₂—CH₂—CH₂F, —CH₂—CF₃, —C(CH₃)₃, —CF₂CH₃, —CHF—CH₃, —CH(CH₃)₂, —CF(CH₃)₂, —C(CH₃)₂—CH₂F, —CH(CH₃)—CHF₂, —CH(CH₃)—CF₃, —C(CH₃)₂—CF₃, —CH₂—CH₂—CN, —CH₂OCH₃, —CH₂—C(CH₃)₂—CN, —C(CH₃)₂—OH, —CH(CH₃)—OCH₃, —C(CH₃)₂—OCH₃, —C(CH₃)₂—CH₂—OCH₃, —CH(OH)—CH₃, —C(CH₃)(OH)—CF₃, —CH═CH₂, —OCH₃, —O—CHF₂, —C(CH₃)(OCH₃)—CH₂—OCH₃, —O—CF₃, —CH(OCH₃)-cyclopropyl, —OH, —O—CH₂CH₃, —O—CH₂CHF₂, —O—CH(CH₃)₂, —O—CH(CF₃)₂, —O—CH₂—OCH₃, —O—CH(CH₃)—OCH₃, —O—CH(CH₃)—CH₂—OCH₃, —O—CH₂—CH₂—O—CH₃, —O—CH₂—CH(CH₃)—OCH₃, —CF(CH₃)—CH₂—OCH₃, —CH(CF₃)—NH₂, —CH(OCH₃)—C(CH₃)₃, —NH—C(O)—CH₃, —NH—SO₂—CH₃, —NH₂, —NHCH₃, —N(CH₃)₂, —NHCH(CH₃)₂, —NHCH₂CH₂CH₃, —NHCH₂C(CH₃)₂OCH₃, cyclopropyl, —CHF— cyclopropyl, —CF(CH₃)-cyclopropyl, —CH(OCH₃)-cyclopropyl, —C(CH₃)(OCH₃)-cyclopropyl, —CF(OCH₃)-cyclopropyl, —NH-cyclohexyl, —NH-cyclopropyl, —NH—N-methylpiperidine, —NH—CH₂-cyclopropyl, —NH—CH₂—CH₂—OCH₃, —N(CH₃)—CH₂—CH₂—OCH₃, —CH(NH₂)—CF₃,

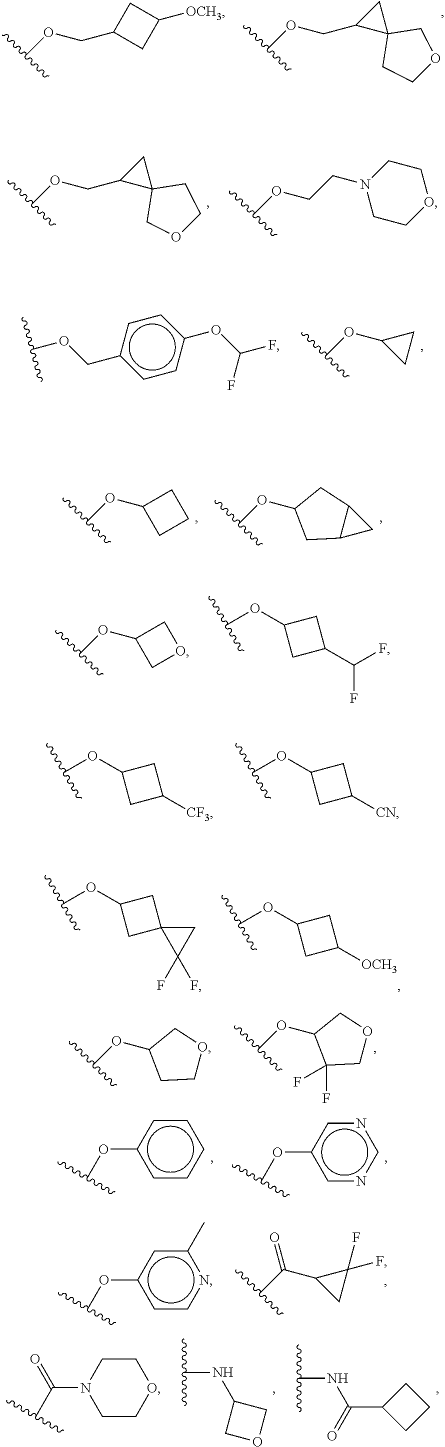

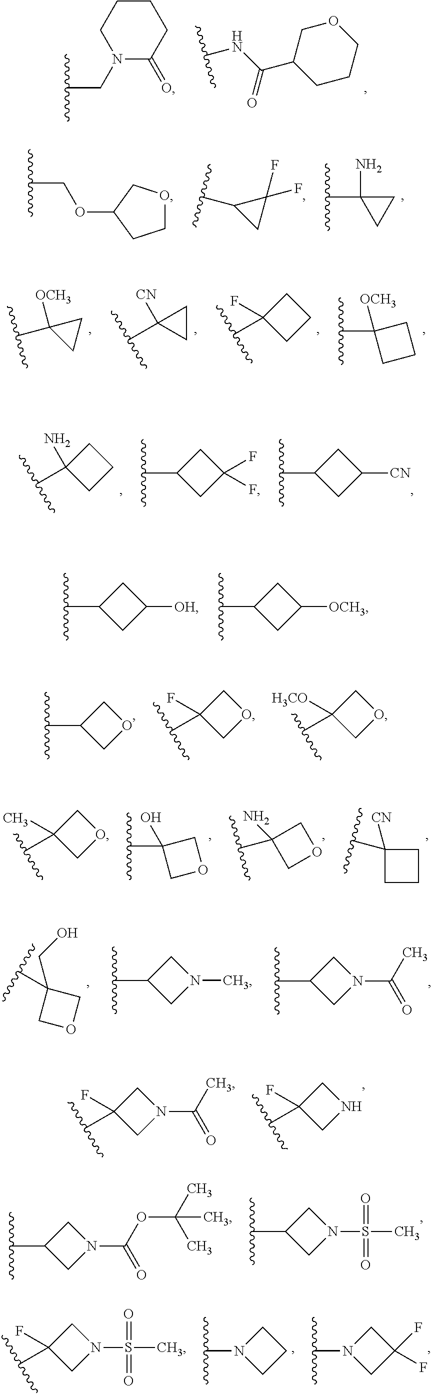

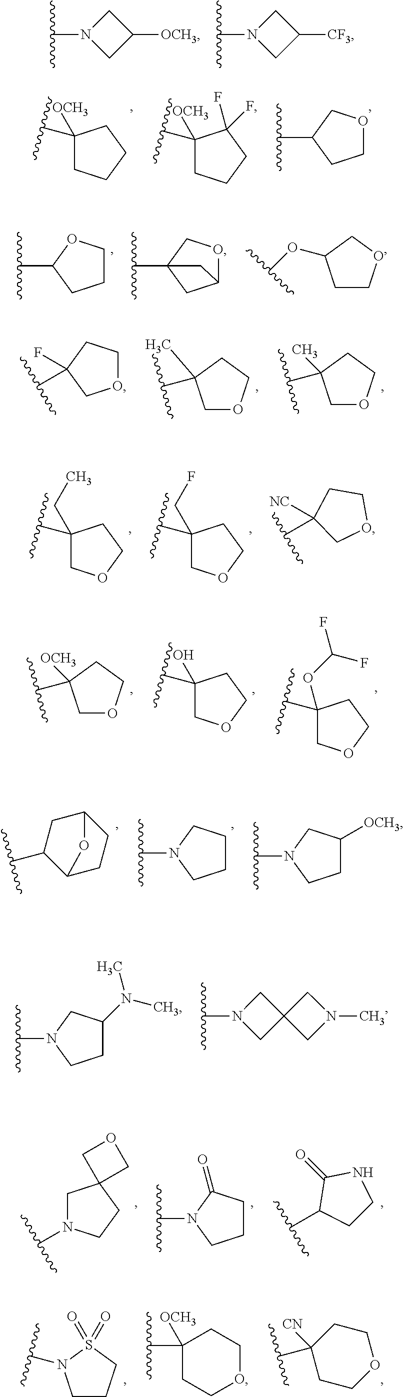

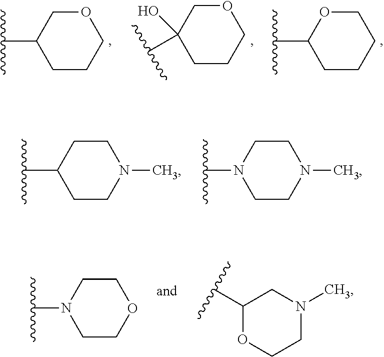

wherein

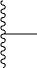

represents a bond to ring C; and the remaining variables are as described in the first aspect or the first, second, third, fourth, fifth, sixth, seventh, eighth or ninth embodiment or any alternative embodiments described therein.

In a seventeenth embodiment, for compounds of formula (I), (II), (III), (IV), (V) or (VI), or pharmaceutically acceptable salts thereof, R¹ is C₁₋₄ alkyl or C₃₋₆ cycloalkyl, wherein the C₁₋₄ alkyl and C₃₋₆ cycloalkyl represented by R¹ are each optionally substituted by one to three R⁸ independently selected from halo, —CN, C₁₋₃alkoxy, C₁₋₃alkyl and C₁₋₃haloalkyl; and the remaining variables are as described in the first aspect or the first, second, third, fourth, fifth, sixth, seventh, eighth, ninth, tenth, eleventh, twelfth, thirteenth, fourteenth, fifteenth or sixteenth embodiment or any alternative embodiments described therein. In an alternative seventeenth embodiment, for compounds of formula (I), (II), (III), (IV), (V) or (VI), or pharmaceutically acceptable salts thereof, R¹ is H, C₁₋₄ alkyl, —OR^{1A}, —NR^{N1}R^{N2}, or C₃₋₆ cycloalkyl, wherein the C₁₋₄ alkyl and C₃₋₆ cycloalkyl represented by R¹ are each optionally substituted by one to three R independently selected from halo, —CN, C₁₋₃alkoxy, C₁₋₃alkyl and C₁₋₃haloalkyl; R^{1A} is C₁₋₄alkyl; R^{N1} and R^{N2} are each independently H or C₁₋₄alkyl; and the remaining variables are as described in the first aspect or the first, second, third, fourth, fifth, sixth, seventh, eighth, ninth, tenth, eleventh, twelfth, thirteenth, fourteenth, fifteenth or sixteenth embodiment or any alternative embodiments described therein.

In an eighteenth embodiment, for compounds of formula (I), (II), (III), (IV), (V) or (VI), or pharmaceutically acceptable salts thereof, R¹ is C₁₋₄ alkyl optionally substituted by one to three R⁸ independently selected from halo, —CN, C₁₋₃alkoxy, C₁₋₃alkyl and C₁₋₃ haloalkyl; and the remaining variables are as described in the first aspect or the first, second, third, fourth, fifth, sixth, seventh, eighth, ninth, tenth, eleventh, twelfth, thirteenth, fourteenth, fifteenth or sixteenth embodiment or any alternative embodiments described therein.

In a nineteenth embodiment, for compounds of formula (I), (II), (III), (IV), (V) or (VI), or pharmaceutically acceptable salts thereof, R⁸, for each occurrence, is independently halo, —CN, C₁₋₃alkoxy; and the remaining variables are as described in the first aspect or the seventeenth or eighteenth embodiment or any alternative embodiments described therein.

In a twentieth embodiment, for compounds of formula (I), (II), (III), (IV), (V) or (VI), or pharmaceutically acceptable salts thereof, each R⁸ is independently selected from F, —CN and —OCH₃; and the remaining variables are as described in the first aspect or the seventeenth or eighteenth embodiment or any alternative embodiments described therein.

In a twenty-first embodiment, for compounds of formula (I), (II), (III), (IV), (V) or (VI), or pharmaceutically acceptable salts thereof, R¹ is selected from —CH₃, —CH₂—CH₃, —CH₂—CHF₂, —CH₂—CH₂—CN, —CH₂—CH₂—OCH₃, cyclopropyl,

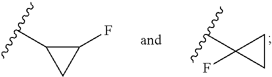 [SMILES: *C1CC1F ; "and" ; *C1(F)CC1 ; ";"]

and the remaining variables are as described in the first aspect or the first, second, third, fourth, fifth, sixth, seventh, eighth, ninth, tenth, eleventh, twelfth, thirteenth, fourteenth, fifteenth or sixteenth embodiment or any alternative embodiments described therein. In an alternative twenty-first embodiment, for compounds of formula (I), (II), (III), (IV), (V) or (VI), or pharmaceutically acceptable salts thereof, R¹ is selected from —H, —CH₃, —CD₃, —CH₂—CH₃, —CH₂—CHF₂, —CH₂—CH₂—CN, —CH₂—CH₂—OCH₃, —OCH₃, —NH₂, —NHCH₃, —N(CH₃)₂, cyclopropyl,

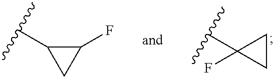 [SMILES: *C1CC1F ; "and" ; *C1(F)CC1 ; ";"]

and the remaining variables are as described in the first aspect or the first, second, third, fourth, fifth, sixth, seventh, eighth, ninth, tenth, eleventh, twelfth, thirteenth, fourteenth, fifteenth or sixteenth embodiment or any alternative embodiments described therein.

In a twenty-second embodiment, for compounds of formula (I), (II), (III), (IV), (V) or (VI), or pharmaceutically acceptable salts thereof, R³ is H, —NR^{N1}R^{N2}, halo, —C(O)—R⁷, —C(O)—OR^{O3}, —SO₂—R⁷, —OR^{O4}, C₁₋₆ alkyl, C₃₋₆cycloalkyl, 4 to 9 membered monocyclic or bicyclic heterocyclyl, wherein the C₁₋₆ alkyl, C₃₋₆cycloalkyl, and 4 to 9 membered monocyclic or bicyclic heterocyclyl represented by R³ are each optionally substituted by one to three R⁹; and the remaining variables are as described in the first aspect or the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth or twenty-first embodiment or any alternative embodiments described therein. In an alternative twenty-second embodiment, for compounds of formula (I), (II), (III), (IV), (V) or (VI), or pharmaceutically acceptable salts thereof, R³ is H, —NR^{N1}R^{N2}, halo, —C(O)—R⁷, —C(O)—OR^{O3}, —SO₂—R⁷, —OR^{O4}, C₁₋₆ alkyl, alkenyl C₃₋₆cycloalkyl, 5 or 6 membered heteroaryl, 4 to 10 membered monocyclic or bicyclic heterocyclyl, wherein the C₁₋₆ alkyl, C₃₋₆cycloalkyl, and 4 to 9 membered monocyclic or bicyclic heterocyclyl represented by R³ are each optionally substituted by one to three R⁹; and the remaining variables are as described in the first aspect or the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth or twenty-first embodiment or any alternative embodiments described therein.

In a twenty-third embodiment, for compounds of formula (I), (II), (III), (IV), (V) or (VI), or pharmaceutically acceptable salts thereof, R³ is C₃₋₆ cycloalkyl or 4 to 9 membered monocyclic or bicyclic heterocyclyl, each optionally substituted by one or three R⁹; and the remaining variables are as described in the first aspect or the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth or twenty-first embodiment or any alternative embodiments described therein. In an alternative twenty-third embodiment, for compounds of formula (I), (II), (III), (IV), (V) or (VI), or pharmaceutically acceptable salts thereof, R³ is C₃₋₆ cycloalkyl or 4 to 10 membered monocyclic or bicyclic heterocyclyl, each optionally substituted by one or three R⁹; and the remaining variables are as described in the first aspect or the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth or twenty-first embodiment or any alternative embodiments described therein.

In a twenty-fourth embodiment, for compounds of formula (I), (II), (III), (IV), (V) or (VI), or pharmaceutically acceptable salts thereof, R³ is selected from azetidinyl, cyclobutyl, cyclopentyl, cyclopropyl, 2,5-diazabicyclo[2.2.1]heptanyl, 3,6-diazabicyclo[3.1.1]heptanyl, 3,6-diazabicyclo[3.2.0]heptanyl, 2,5-diazabicyclo [2.2.2]octanyl, 3,8-diazabicyclo[3.2.1]octanyl, morpholinyl, octahydropyrrolo[3,4-c]pyrrolyl, 2-oxa-5-azabicylo[2.2.1]heptanyl, 6-oxa-3-azabicyclo[3.1.1]heptanyl, 8-oxa-3-azabicyclo[3.2.1]octanyl, 2λ²,6-diazaspiro[3.3]heptanyl, 1λ²,7λ²-diazaspiro[4.4]nonanyl, oxetanyl, piperidinyl, piperazinyl, piperazine-2-one-yl, pyrrolidinyl, pyrrolidine-2-one-yl and tetrahydropyranyl, each of which is optionally substituted by one to three R⁹; and the remaining variables are as described in the first aspect or the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth or twenty-first embodiment or any alternative embodiments described therein. In some embodiments, R³ is selected from pyrrolidinyl, morpholinyl, 6-oxa-3-azabicyclo[3.1.1]heptanyl, and 3,6-diazabicyclo[3.1.1]heptanyl, each of which is optionally substituted by one to three R⁹. In some embodiments, R³ is pyrrolidinyl, optionally substituted by one to three R⁹. In an alternative twenty-fourth embodiment, for compounds of formula (I), (II), (III), (IV), (V) or (VI), or pharmaceutically acceptable salts thereof, R³ is selected from pyrazoyl, pyridinyl, azetidinyl, cyclobutyl, cyclopentyl, cyclopropyl, 2-oxaspiro[3.3]heptanyl, 1,7-diazaspiro[4.4]nonanyl, 2,7-diazaspiro[3.5]nonanyl, 7-oxa-2-azaspiro[3.5]nonanyl, 1-oxa-7-azaspiro[4.4]nonanyl, 2,6-diazaspiro[3.4]octanyl, 2-oxa-6-azaspiro[3.4]octanyl, 6-oxa-2-azaspiro[3.4]octanyl, 2,7-diazaspiro[4.4]nonanyl, 1,6-diazaspiro[3.3]heptanyl, 1-oxa-6-azaspiro[3.3]heptanyl, 3,6-diazabicyclo[3.2.0]heptanyl, 3,9-diazabicyclo[3.3.1]nonanyl, 6-oxa-2,9-diazaspiro[4.5]decanyl, 1,6-diazaspiro[3.4]octanyl, 5-azaspiro[2.4]heptanyl, 1,6-diazaspiro[3.4]octanyl, 1,7-diazaspiro[4.4]nonanyl, 2-oxa-7-azaspiro[4.4]nonanyl, octahydropyrano[2,3-c]pyrrolyl, octahydro-1H-pyrrolo[3,4-b]pyridinyl, octahydropyrrolo[3,4-b][1,4]oxazinyl, octahydropyrrolo[3,4-b]pyrrolyl, 3-oxa-6-azabicyclo[3.1.1]heptanyl, hexahydro-1H-furo[3,4-c]pyrrolyl, 1,4-oxazepanyl, 6-oxa-2-azaspiro[3.5]nonanyl, 5-oxa-2-azaspiro[3.4]octanyl, 8-oxa-3-azabicyclo[3.2.1]octanyl, 3-oxa-7-azabicyclo[3.3.1]nonanyl, 2,5-diazabicyclo[2.2.1]heptanyl, 3,6-diazabicyclo[3.1.1]heptanyl, 3,6-diazabicyclo[3.2.0]heptanyl, 2,5-diazabicyclo [2.2.2]octanyl, 3,8-diazabicyclo[3.2.1]octanyl, morpholinyl, octahydropyrrolo[3,4-c]pyrrolyl, 2-oxa-5-azabicylo[2.2.1]heptanyl, 6-oxa-3-azabicyclo[3.1.1]heptanyl, 8-oxa-3-azabicyclo[3.2.1]octanyl, 2λ²,6-diazaspiro[3.3]heptanyl, 1λ²,7λ²-diazaspiro[4.4]nonanyl, oxetanyl, piperidinyl, piperazinyl, piperazine-2-one-yl, pyrrolidinyl, pyrrolidine-2-one-yl and tetrahydropyranyl, each of which is optionally substituted by one to three R⁹; and the remaining variables are as described in the first aspect or the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth or twenty-first embodiment or any alternative embodiments described therein.

In a twenty-fifth embodiment, for compounds of formula (I), (II), (III), (IV), (V) or (VI), or pharmaceutically acceptable salts thereof, R³ is selected from:

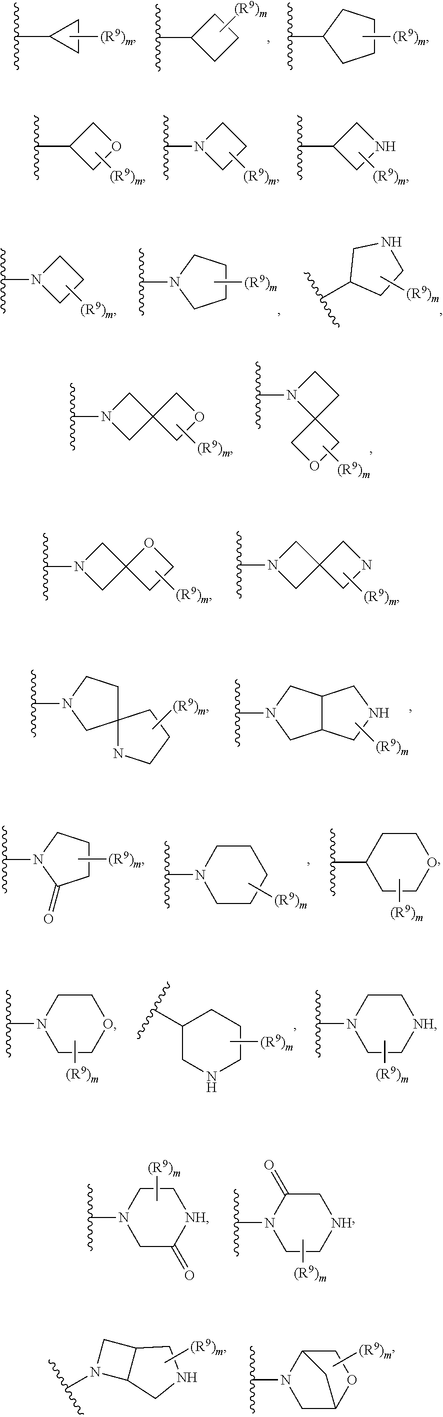

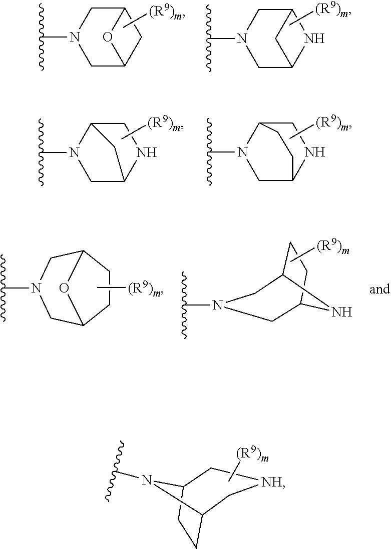

wherein

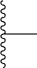

represents a bond to ring B, and m is 0, 1, 2 or 3; and the remaining variables are as described in the first aspect or the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth or twenty-first embodiment or any alternative embodiments described therein. In some embodiments, R³ is selected from:

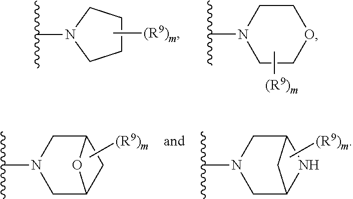

In an alternative twenty-fifth embodiment, for compounds of formula (I), (II), (III), (IV), (V) or (VI), or pharmaceutically acceptable salts thereof, R³ is selected from:

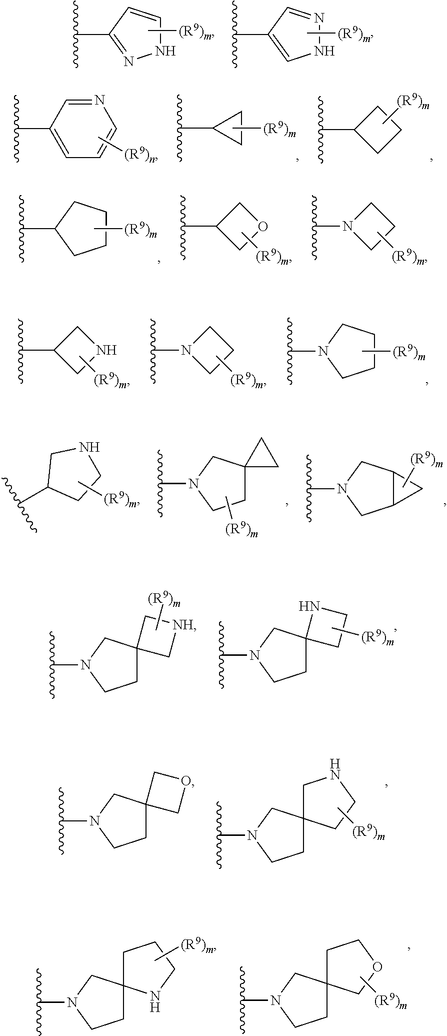

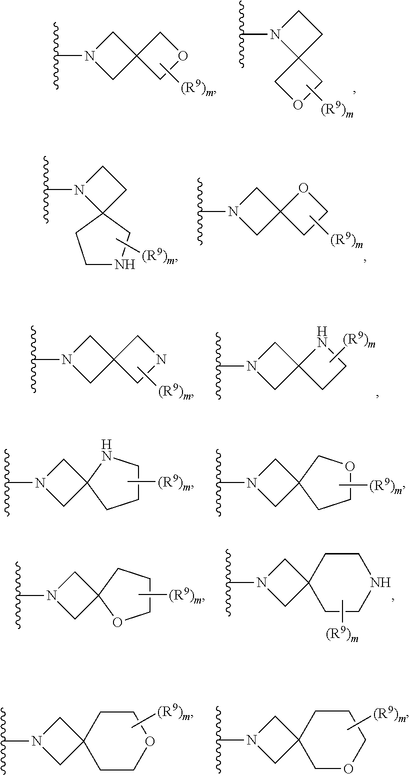

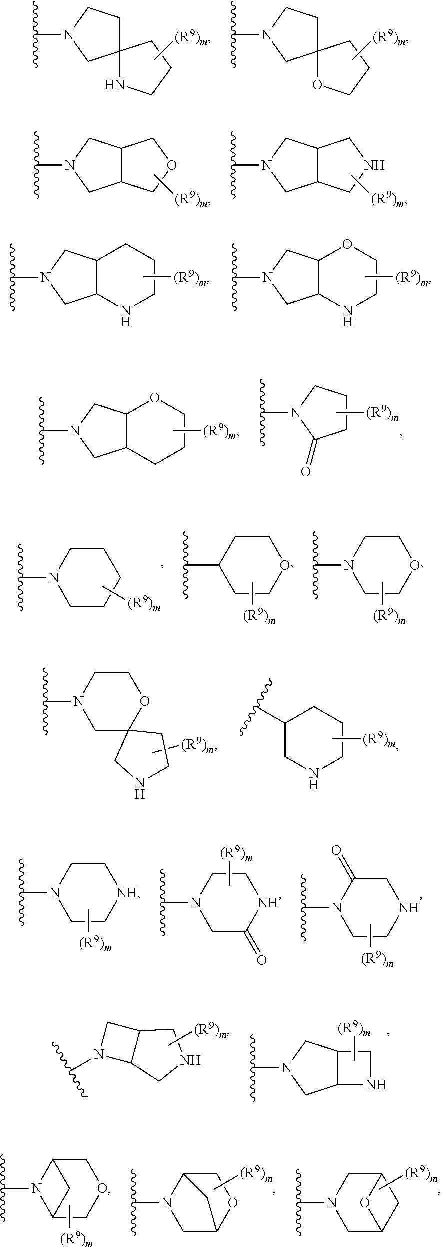

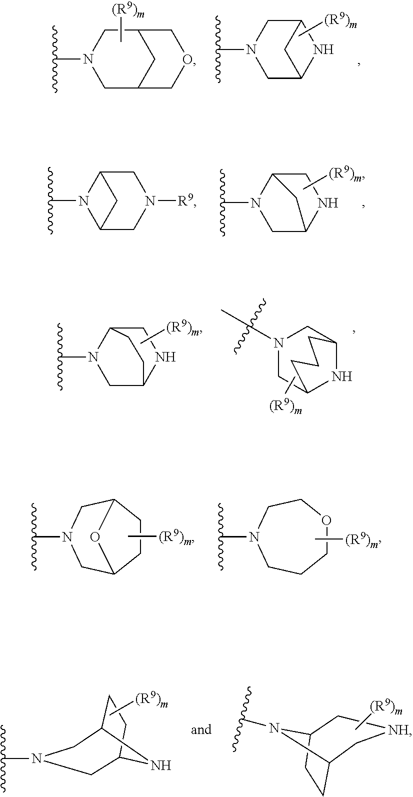

wherein

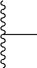

represents a bond to ring B, and m is 0, 1, 2, 3 or 4; and the remaining variables are as described in the first aspect or the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth or twenty-first embodiment or any alternative embodiments described therein.

In a twenty-sixth embodiment, for compounds of formula (I), (II), (III), (IV), (V) or (VI), or pharmaceutically acceptable salts thereof, R³ is selected from:

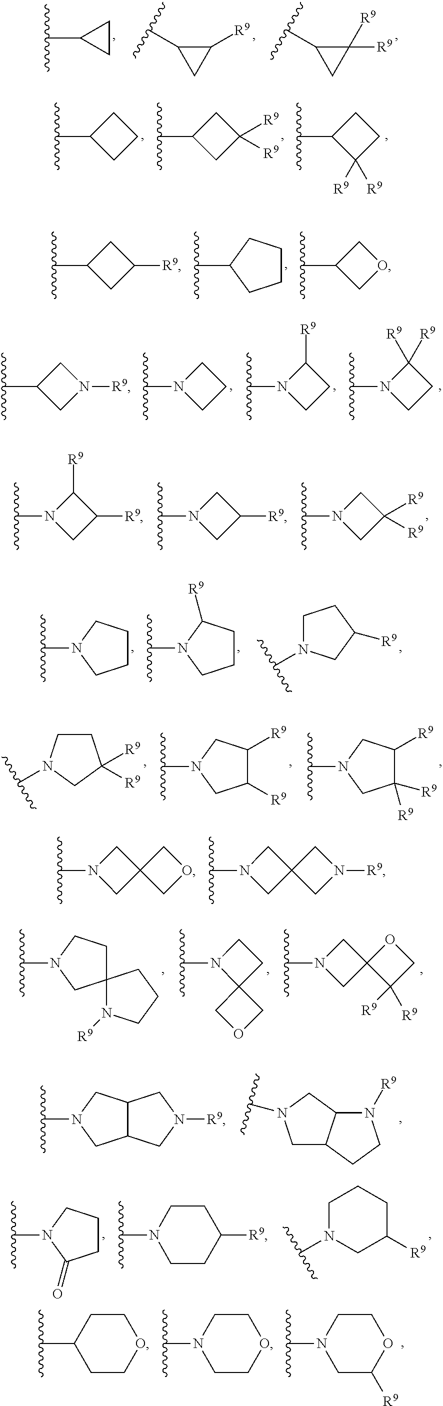

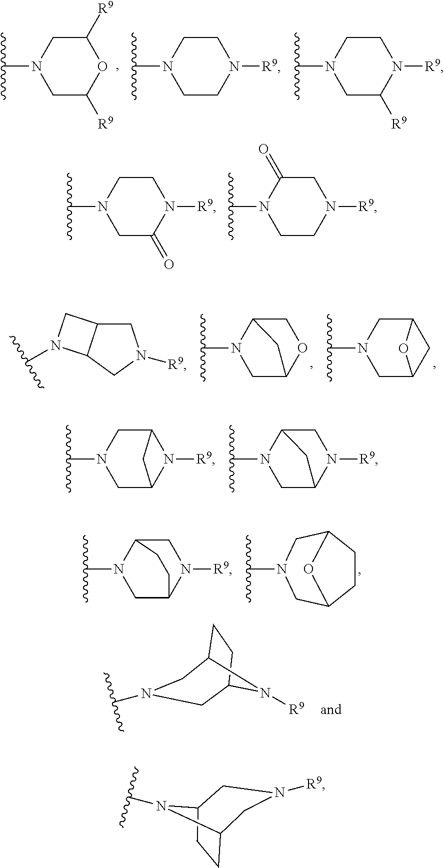

wherein

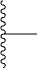

represents a bond to ring B; and the remaining variables are as described in the first aspect or the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth or twenty-first embodiment or any alternative embodiments described therein. In some embodiments, R³ is selected from:

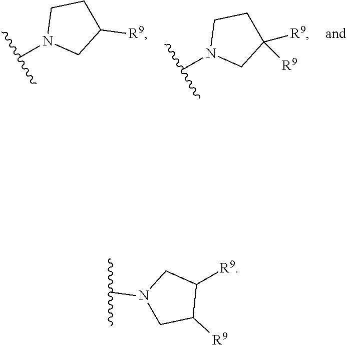

In an alternative twenty-sixth embodiment, for compounds of formula (I), (II), (III), (IV), (V) or (VI), or pharmaceutically acceptable salts thereof, R³ is selected from:

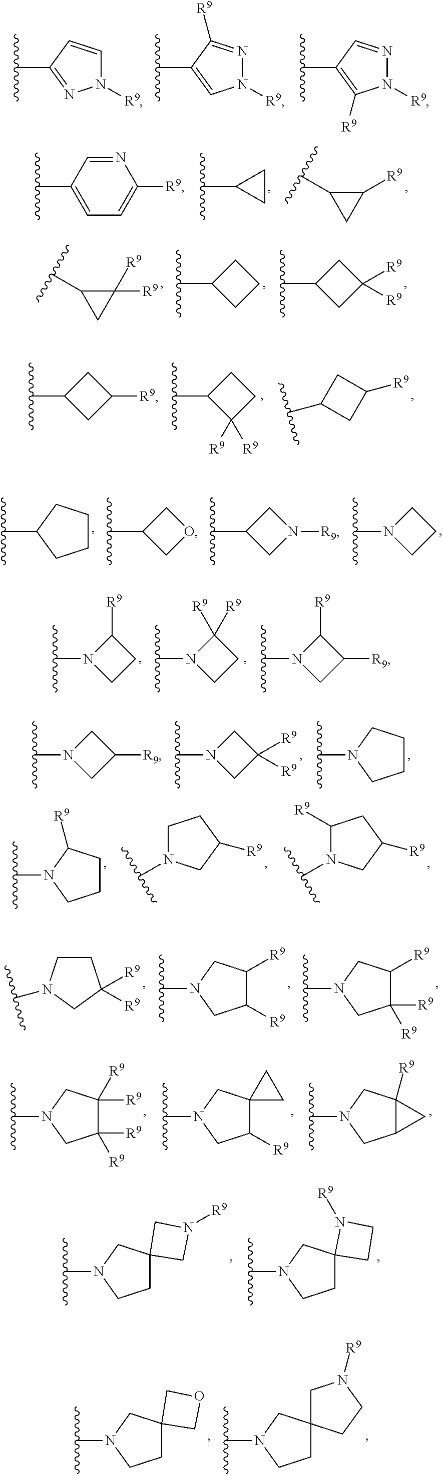

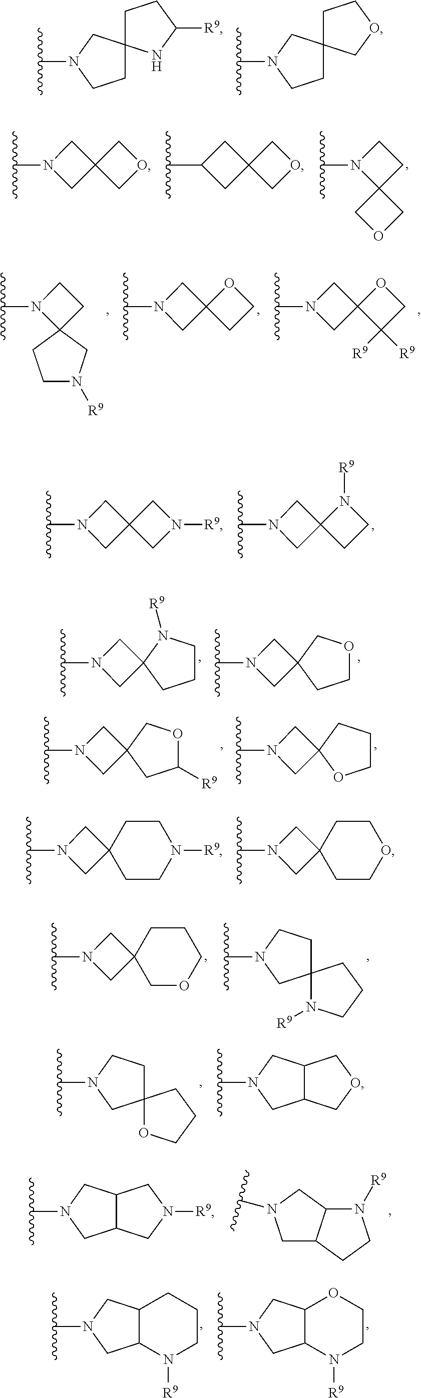

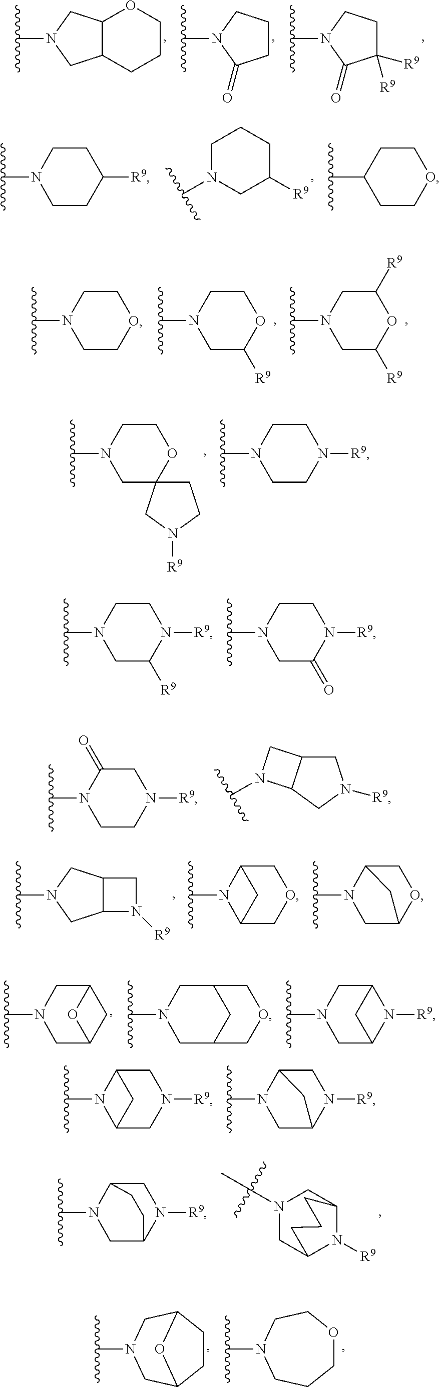

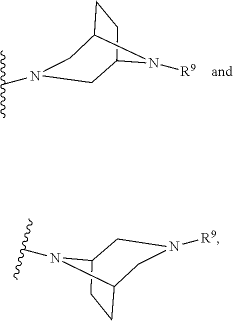

wherein

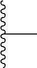

represents a bond to ring B; and the remaining variables are as described in the first aspect or the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth or twenty-first embodiment.

In a twenty-seventh embodiment, for compounds of formula (I), (II), (III), (IV), (V) or (VI), or pharmaceutically acceptable salts thereof, R³ is H, —NR^{N1}R^{N2}, halo, —C(O)—R⁷, —C(O)—OR^{O3}, —SO₂—R⁷, —OR^{O4}, or C₁₋₆ alkyl optionally substituted with one to three R⁹; and the remaining variables are as described in the first aspect or the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth or twenty-first embodiment or any alternative embodiments described therein.

In a twenty-eighth embodiment, for compounds of formula (I), (II), (III), (IV), (V) or (VI), or pharmaceutically acceptable salts thereof, each R⁹ is independently halo, OH, —OC₁₋₄ alkyl, —NR^{a2}R^{a3}, —CN, —C(O)—OR^{a1}, —SO₂—R^{a1}, C₁₋₄ alkyl, or C₃₋₆ cycloalkyl, wherein the C₁₋₄ alkyl and C₃₋₆ cycloalkyl represented by R⁹ are each optionally substituted by one to three substituents independently selected from halo and C₁₋₃ alkoxy; and R^{a1}, R^{a2} and R^{a3} are each independently C₁₋₃ alkyl; and the remaining variables are as described in the first aspect or the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth or twenty-seventh embodiment or any alternative embodiments described therein. In some embodiments, each R⁹ is independently halo, —OR^{a1}, —NR^{a2}R^{a3}, —CN, —C(O)—OR^{a1}, —SO₂—R^{a1}, C₁₋₄ alkyl, C₃₋₆ cycloalkyl, or 4 to 7 membered monocyclic heterocyclyl, wherein the C₁₋₄ alkyl and C₃₋₆ cycloalkyl represented by R⁹ are each optionally substituted by one to three substituents independently selected from halo and C₁₋₃ alkoxy; and R^{a1}, R^{a2} and R^{a3} are each independently H or C₁₋₃ alkyl. In an alternative twenty-eighth embodiment, for compounds of formula (I), (II), (III), (IV), (V) or (VI), or pharmaceutically acceptable salts thereof, R⁹ is independently halo, —OR^{a1}, —NR^{a2}R^{a3}, —CN, —C(O)—OR^{a1}, —SO₂—R^{a1}, C₁₋₄ alkyl, C₃₋₆ cycloalkyl, or 4 to 10 membered monocyclic or bicyclic heterocyclyl, wherein the C₁₋₄ alkyl, 4 to 10 membered monocyclic or bicyclic heterocyclyl, and C₃₋₆ cycloalkyl represented by R⁹ are each optionally substituted by one to three substituents independently selected from halo, OH, —CN, C₁₋₄ alkyl, and C₁₋₃ alkoxy; and R^{a1}, R^{a2} and R^{a3} are each independently H, C₃₋₄cycloalkyl, or C₁₋₃ alkyl, wherein the C₃₋₄cycloalkyl represented by R^{a1}, R^{a2} and R^{a3} is optionally substituted with C₁₋₄alkyl, and wherein the C₁₋₃ alkyl represented by R^{a1}, R^{a2} and R^{a3} is optionally substituted with phenyl; and the remaining variables are as described in the first aspect or the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth or twenty-seventh embodiment or any alternative embodiments described therein.

In a twenty-ninth embodiment, for compounds of formula (I), (II), (III), (IV), (V) or (VI), or pharmaceutically acceptable salts thereof, each R⁹ is independently selected from F, OH, —OCH₃, —OCHF₂, —OCF₃, —N(CH₃)₂, —CN, —CH₃, —CF₃, —C(O)—OCH₂CH₃, —SO₂—CH₃, —CH₂—OCH₃, —CH₂—CH₂—OCH₃, —CH₂—CH₃, cyclopropyl, —CHF₂ and —CH₂—CF₃; and the remaining variables are as described in the first aspect or the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth or twenty-seventh embodiment or any alternative embodiments described therein. In some embodiments, each R⁹ is independently selected from F, OH, —OCH₃, —OCHF₂, —OCF₃, —NHCH₃, —NH₂, —N(CH₃)₂, —N(CH₂CH₃)₂, —N(CH₃)(CH₂CH₃), —NHCH(CH₃)₂, —CN, —CH₃, —CF₃, —C(O)—OCH₂CH₃, —SO₂—CH₃, —CH₂—OCH₃, —CH₂—CH₂—OCH₃, —CH₂—CH₃, pyrrolidinyl, morpholinyl, cyclopropyl, —CHF₂, and —CH₂—CF₃. In an alternative twenty-ninth embodiment, for compounds of formula (I), (II), (III), (IV), (V) or (VI), or pharmaceutically acceptable salts thereof, each R⁹ is independently selected from F, OH, —OCH₃, —OCH₂CH₃, —OCH(CH₃)₂, —OCHF₂, —OCF₃, —OCD₃, —O— cyclopropyl, —NHCH₃, —NH₂, —N(CH₃)₂, —N(CD₃)₂, —N(CH₂CH₃)₂, —N(CH₃)(CH₂CH₃), —NHCH(CH₃)₂, —NHCH₂CH₃, —CN, —CH₃, —CH₂F, —CF₃, —C(O)—OCH₂CH₃, —SO₂—CH₃, —CH₂—OCH₃, —CH₂—CH₂—OCH₃, —CH₂—CH₃, —CH₂—CN, pyrrolidinyl, morpholinyl, azetidinyl, cyclopropyl, —CHF₂, —CH₂—CF₃,

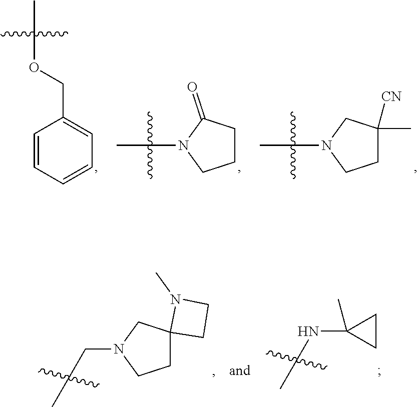

and the remaining variables are as described in the first aspect or the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth or twenty-seventh embodiment or any alternative embodiments described therein.

In a thirtieth embodiment, for compounds of formula (I), (II), (III), (IV), (V) or (VI), or pharmaceutically acceptable salts thereof, two of R⁹, taken together with their intervening atoms, form a 4 to 6 membered monocyclic heterocyclyl optionally substituted by one to two substituents independently selected from halo, C₁₋₄ alkyl and C₁₋₄ haloalkyl; and the remaining variables are as described in the first aspect or the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth or twenty-seventh embodiment or any alternative embodiments described therein.

In a thirty-first embodiment, for compounds of formula (I), (II), (III), (IV), (V) or (VI), or pharmaceutically acceptable salts thereof, R³ is selected from H, Cl, —CH₃, —CH₂F, —CHF₂, —CF₃, —CH₂—CH₃, —CHF—CH₃, —CH(CH₃)₂, —CHF—CH₂F, —CH₂—CH₂—CN, —CH₂—CH₂—CH₃, —CH₂—CH(CH₃)₂, —CH₂-cyclopropyl, —CH₂—OCH₃, —CH(CH₃)—CH₂—CH₃, —CH(CH₃)—CF₃, —CH(CH₃)—OCH₃, —N(CH₃)₂, —OCH₃, —N(CH₃)—CH₂—CH₂—OCH₃, —C(O)—OCH₃, —SO₂—CH₃, —O—CH(CH₃)₂, —O—CH₂—CH₂—OCH₃,

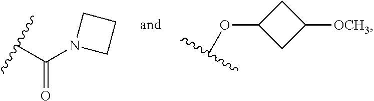 [SMILES: *C(=O)N1CCC1 ; *OC1CC(OC)C1]

wherein

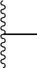

represents a bond to ring B; and the remaining variables are as described in the first aspect or the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth or twenty-first embodiment or any alternative embodiments described therein. In an alternative thirty-first embodiment, for compounds of formula (I), (II), (III), (IV), (V) or (VI), or pharmaceutically acceptable salts thereof, R³ is selected from H, Cl, —CN, —CH₃, —CH₂F, —CHF₂, —CF₃, —CH₂—CH₃, —CH₂CF₃, —CHF—CH₃, —CH(CH₃)₂, —CH(CF₃)₂, —CHF—CH₂F, —CH₂—CH₂—CN, —CH₂—CH₂—CH₃, —CH(CH₃)—CH₂—CH₃, —CH₂—CH(CH₃)₂, —CH₂-cyclopropyl, —CH₂— morpholinyl, —CH₂OH, —CH₂—OCH₃, —C(CH₃)—CH₂—CH₃, —CH(CH₃)—CF₃, —CH(CH₃)—OCH₃, —CH₂N(CH₃)₂, —CH═CH₂, —NH₂, —NHCH₃, —NHCH₂CH₃, —N(CH₃)₂, —OCH₃, —N(CH₃)—CH₂—CH₂—OCH₃, —N(CH₃)CH₂C(CH₃)₂OCH₃, —C(O)—OCH₃, —C(O)—OCH₂CH₃, —SO₂—CH₃, —O—CH(CH₃)₂, —O—CH₂—CH₂—OCH₃,

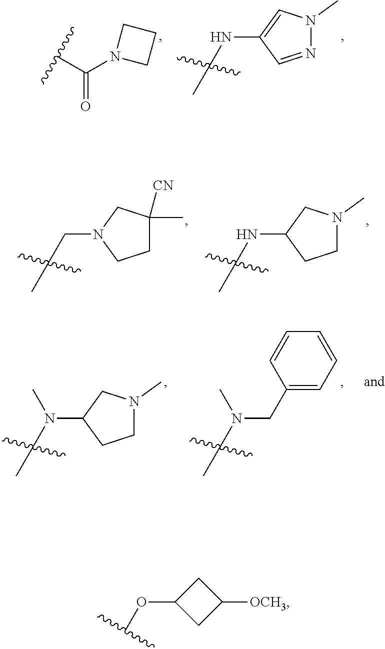

wherein

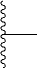

represents a bond to ring B; and the remaining variables are as described in the first aspect or the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth or twenty-first embodiment or any alternative embodiments described therein.

In a thirty-second embodiment, for compounds of formula (I), (II), (III), (IV), (V) or (VI), or pharmaceutically acceptable salts thereof, each R⁷ is independently C₁₋₆ alkyl, C₃₋₆ cycloalkyl or 4 to 6 membered monocyclic heterocyclyl, wherein the C₁₋₆ alkyl, C₃₋₆ cycloalkyl and 4 to 6 membered monocyclic heterocyclyl represented by R⁷ are each optionally substituted by one to three substituents independently selected from halo, C₁₋₃alkyl and C₁₋₃ haloalkyl; and the remaining variables are as described in the first aspect or the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth, twenty-seventh, twenty-eighth, twenty-ninth, thirtieth or thirty-first embodiment or any alternative embodiments described therein.

In a thirty-third embodiment, for compounds of formula (I), (II), (III), (IV), (V) or (VI), or pharmaceutically acceptable salts thereof, each R⁷ is independently selected from —CH₃,

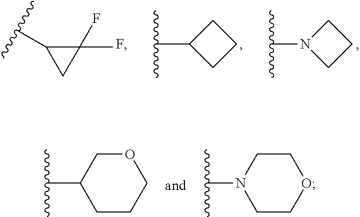

and the remaining variables are as described in the first aspect or the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth, twenty-seventh, twenty-eighth, twenty-ninth, thirtieth or thirty-first embodiment or any alternative embodiments described therein.

In a thirty-fourth embodiment, for compounds of formula (I), (II), (III), (IV), (V) or (VI), or pharmaceutically acceptable salts thereof, each R^{O1} is independently H, C₁₋₆ alkyl, 3 to 6 membered monocyclic or bicyclic carbocyclyl, 4 to 6 membered monocyclic heterocyclyl, or 6 membered heteroaryl, wherein the C₁₋₆ alkyl, 3 to 6 membered monocyclic or bicyclic carbocyclyl, 4 to 6 membered monocyclic heterocyclyl and 6 membered heteroaryl represented by R^{O1} are each optionally substituted by one to three R^{O2}; and each R^{O2} is independently halo, OH, —CN, C₁₋₄ alkoxy, C₁₋₄ alkyl, 3 to 5 membered monocyclic carbocyclyl, 4 to 7 membered monocyclic or bicyclic heterocyclyl or phenyl, wherein the C₁₋₄ alkyl, 3 to 5 membered monocyclic carbocyclyl, 4 to 7 membered monocyclic or bicyclic heterocyclyl and phenyl are each optionally substituted with C₁₋₃ alkoxy, C₁₋₃ haloalkoxy, or one to three halo; and the remaining variables are as described in the first aspect or the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth, twenty-seventh, twenty-eighth, twenty-ninth, thirtieth, thirty-first, thirty-second or thirty-third embodiment or any alternative embodiments described therein.

In a thirty-fifth embodiment, for compounds of formula (I), (II), (III), (IV), (V) or (VI), or pharmaceutically acceptable salts thereof, the 4 to 7 membered monocyclic or bicyclic heterocyclyl or 6 membered heteroaryl represented by R^{O1} or R^{O2} are each independently selected from morpholino, oxetanyl, pyridinyl, pyrimidinyl, tetrahydrofuranyl, tetrahydropyranyl, morpholinyl, azetidinyl, oxaspiro[2.4]heptane, pyrrolidinyl and piperidinyl; and the remaining variables are as described in the first aspect or the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth, twenty-seventh, twenty-eighth, twenty-ninth, thirtieth, thirty-first, thirty-second or thirty-third embodiment or any alternative embodiments described therein.

In a thirty-sixth embodiment, for compounds of formula (I), (II), (III), (IV), (V) or (VI), or pharmaceutically acceptable salts thereof, each R^{O2} is independently selected from F, —CN, OH, —OCH₃, —CH₃, —CHF₂, —CF₃,

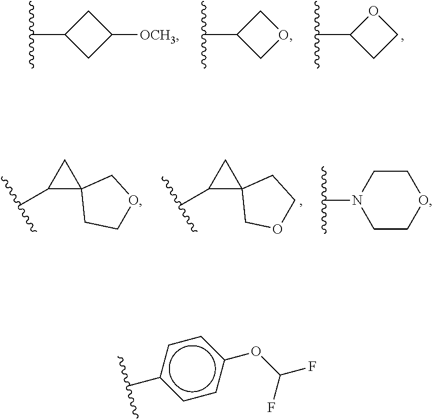

wherein

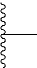

represents a bond to R^{O1}; and the remaining variables are as described in the first aspect or the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth, twenty-seventh, twenty-eighth, twenty-ninth, thirtieth, thirty-first, thirty-second, thirty-third, thirty-fourth or thirty-fifth embodiment or any alternative embodiments described therein.

In a thirty-seventh embodiment, for compounds of formula (I), (II), (III), (IV), (V) or (VI), or pharmaceutically acceptable salts thereof, each R^{O1} is independently selected from H, —CH₃, —CHF₂, —CF₃, —CH₂—CH₃, —CH₂—CHF₂, —CH₂—CF₃, —CH₂—CH₂—CH₃, —CH(CH₃)₂, —CH(CF₃)₂, —CH₂—CH₂—OCH₃, —CH(CH₃)—CH₂—OCH₃, —CH₂—CH(CH₃)—OCH₃,

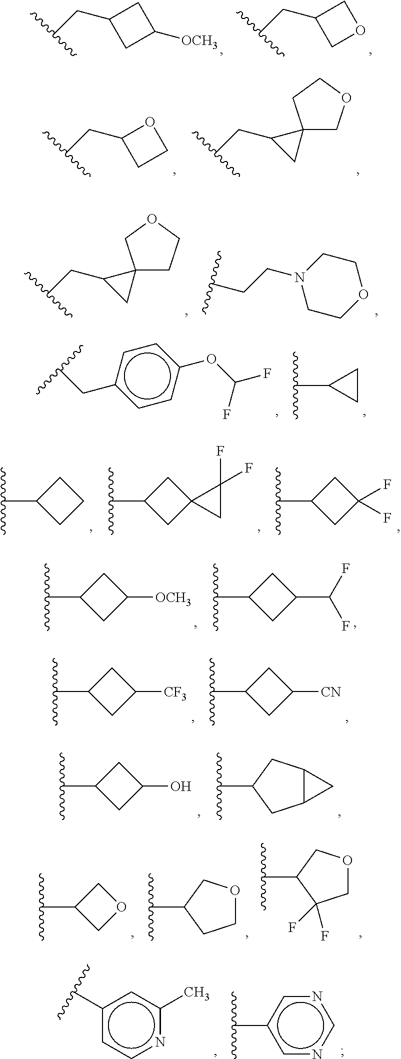

and the remaining variables are as described in the first aspect or the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth, twenty-seventh, twenty-eighth, twenty-ninth, thirtieth, thirty-first, thirty-second or thirty-third embodiment or any alternative embodiments described therein.

In a thirty-eighth embodiment, for compounds of formula (I), (II), (III), (IV), (V) or (VI), or pharmaceutically acceptable salts thereof, each R^{O3} is independently C₁₋₆ alkyl optionally substituted by one to three substituents independently selected from halo and C₁₋₄ haloalkyl; and the remaining variables are as described in the first aspect or the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth, twenty-seventh, twenty-eighth, twenty-ninth, thirtieth, thirty-first, thirty-second, thirty-third, thirty-fourth, thirty-fifth, thirty-sixth or thirty-seventh embodiment or any alternative embodiments described therein.

In a thirty-ninth embodiment, for compounds of formula (I), (II), (III), (IV), (V) or (VI), or pharmaceutically acceptable salts thereof, each R^{O3} is independently —CH₃, —CH₂CH₃ or —C(CH₃)₃; and the remaining variables are as described in the first aspect or the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth, twenty-seventh, twenty-eighth, twenty-ninth, thirtieth, thirty-first, thirty-second, thirty-third, thirty-fourth, thirty-fifth, thirty-sixth or thirty-seventh embodiment or any alternative embodiments described therein.

In a fortieth embodiment, for compounds of formula (I), (II), (III), (IV), (V) or (VI), or pharmaceutically acceptable salts thereof, R^{O4} is C₁₋₄ alkyl or C₃₋₆ cycloalkyl, each optionally substituted by one to three substituents independently selected from halo and C₁₋₃ alkoxy; and the remaining variables are as described in the first aspect or the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth, twenty-seventh, twenty-eighth, twenty-ninth, thirtieth, thirty-first, thirty-second, thirty-third, thirty-fourth, thirty-fifth, thirty-sixth, thirty-seventh, thirty-eighth or thirty-ninth embodiment or any alternative embodiments described therein.

In a forty-first embodiment, for compounds of formula (I), (II), (III), (IV), (V) or (VI), or pharmaceutically acceptable salts thereof, R^{O4} is selected from —CH₃, —CH₂—CH₃, —CH(CH₃)₂, —CH₂—CH₂—OCH₃, and

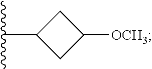 [SMILES: *C1CC(OC)C1]

and the remaining variables are as described in the first aspect or the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth, twenty-seventh, twenty-eighth, twenty-ninth, thirtieth, thirty-first, thirty-second, thirty-third, thirty-fourth, thirty-fifth, thirty-sixth, thirty-seventh, thirty-eighth or thirty-ninth embodiment or any alternative embodiments described therein.

In a forty-second embodiment, for compounds of formula (I), (II), (III), (IV), (V) or (VI), or pharmaceutically acceptable salts thereof, R^{N1} and R^{N2} each independently is H, C₃₋₆ cycloalkyl or C₁₋₄ alkyl optionally substituted with C₁₋₃ alkoxy; and the remaining variables are as described in the first aspect or the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth, twenty-seventh, twenty-eighth, twenty-ninth, thirtieth, thirty-first, thirty-second, thirty-third, thirty-fourth, thirty-fifth, thirty-sixth, thirty-seventh, thirty-eighth, thirty-ninth, fortieth or forty-first embodiment or any alternative embodiments described therein.

In a forty-third embodiment, for compounds of formula (I), (II), (III), (IV), (V) or (VI), or pharmaceutically acceptable salts thereof, R^{N1} and R^{N2} each independently represent H, —CH₃, —CH(CH₃)₂, —CH₂—CH₂—OCH₃ or cyclohexyl; and the remaining variables are as described in the first aspect or the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth, twenty-seventh, twenty-eighth, twenty-ninth, thirtieth, thirty-first, thirty-second, thirty-third, thirty-fourth, thirty-fifth, thirty-sixth, thirty-seventh, thirty-eighth, thirty-ninth, fortieth or forty-first embodiment or any alternative embodiments described therein.

In a forty-fourth embodiment, for compounds of formula (I), (II), (III), (IV), (V) or (VI), or pharmaceutically acceptable salts thereof, R^{N3} is H; and the remaining variables are as described in the first aspect or the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth, twenty-seventh, twenty-eighth, twenty-ninth, thirtieth, thirty-first, thirty-second, thirty-third, thirty-fourth, thirty-fifth, thirty-sixth, thirty-seventh, thirty-eighth, thirty-ninth, fortieth, forty-first, forty-second or forty-third embodiment or any alternative embodiments described therein.

In a forty-fifth embodiment, for compounds of formula (I), (II), (III), (IV), (V) or (VI), or pharmaceutically acceptable salts thereof, R^{N4} is H; and the remaining variables are as described in the first aspect or the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth, twenty-seventh, twenty-eighth, twenty-ninth, thirtieth, thirty-first, thirty-second, thirty-third, thirty-fourth, thirty-fifth, thirty-sixth, thirty-seventh, thirty-eighth, thirty-ninth, fortieth, forty-first, forty-second, forty-third or forty-fourth embodiment or any alternative embodiments described therein.

In a forty-sixth embodiment, for compounds of formula (I), (II), (III), (IV), (V) or (VI), or pharmaceutically acceptable salts thereof, R⁴ is H; and the remaining variables are as described in the first aspect or the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth, twenty-seventh, twenty-eighth, twenty-ninth, thirtieth, thirty-first, thirty-second, thirty-third, thirty-fourth, thirty-fifth, thirty-sixth, thirty-seventh, thirty-eighth, thirty-ninth, fortieth, forty-first, forty-second, forty-third, forty-fourth or forty-fifth embodiment or any alternative embodiments described therein. In an alternative forty-sixth embodiment, for compounds of formula (I), (II), (III), (IV), (V) or (VI), or pharmaceutically acceptable salts thereof, R⁴ is H or —CH₃; and the remaining variables are as described in the first aspect or the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth, twenty-seventh, twenty-eighth, twenty-ninth, thirtieth, thirty-first, thirty-second, thirty-third, thirty-fourth, thirty-fifth, thirty-sixth, thirty-seventh, thirty-eighth, thirty-ninth, fortieth, forty-first, forty-second, forty-third, forty-fourth or forty-fifth embodiment or any alternative embodiments described therein.

In a forty-seventh embodiment, the compound is represented by formula (VII) or (VIII):

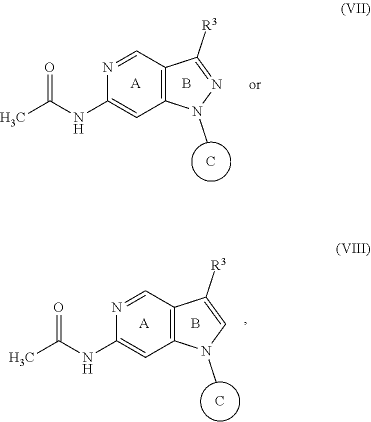

or a pharmaceutically acceptable salt thereof, wherein ring C is 6 membered heteroaryl optionally substituted by one to three R^{C}; each R^{C} is independently —OR^{O1}, C₁₋₄ alkyl or 5 membered heterocyclyl, wherein the C₁₋₄ alkyl and 5 membered heterocyclyl represented by R^{C} are each optionally substituted with one to three R^{C1}; each R^{C1} is independently halo or —OR^{O1}; R³ is H, C₁₋₄ alkyl, C₃₋₅ cycloalkyl, or 5 to 7 membered monocyclic or bicyclic heterocyclyl, wherein the C₁₋₄ alkyl, C₃₋₅ cycloalkyl and 5 to 7 membered monocyclic or bicyclic heterocyclyl represented by R³ are each optionally substituted by one to three R⁹; each R⁹ is independently —OR^{O1}, —NR^{N1}R^{N2}, 5 to 7 membered monocyclic heterocyclyl, or C₁₋₄ alkyl; each R^{O1} is independently H, C₁₋₄ alkyl or C₃₋₅ cycloalkyl, wherein the C₁₋₄ alkyl and C₃₋₅ cycloalkyl are each optionally substituted by R^{O2}; each R^{O2} is independently —CN or C₁₋₄ alkoxy; and R^{N1} and R^{N2} are each independently H or C₁₋₃ alkyl. In some embodiments, R³ is a 5 to 7 membered monocyclic or bicyclic heterocyclyl linked to ring B through a ring N atom. In some embodiments, each R⁹ is independently —OR^{O1}, —NR^{N1}R^{N2}, or C₁₋₄ alkyl. In an alternative forty-seventh embodiment, the compound is represented by formula (VII′) or (VIII′):

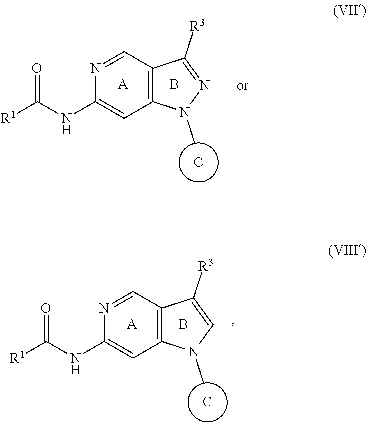

or a pharmaceutically acceptable salt thereof, wherein ring C is 6 membered heteroaryl optionally substituted by one to three R^{C}; each R^{C} is independently —OR^{O1}, C₁₋₄ alkyl or 5 membered heterocyclyl, wherein the C₁₋₄ alkyl and 5 membered heterocyclyl represented by R^{C} are each optionally substituted with one to three R^{C1}; each R^{C1} is independently halo or —OR^{O1}; R¹ is H or —CH₃; R³ is H, C₁₋₄ alkyl, C₃₋₅ cycloalkyl, or 5 to 7 membered monocyclic or bicyclic heterocyclyl, wherein the C₁₋₄ alkyl, C₃₋₅ cycloalkyl and 5 to 7 membered monocyclic or bicyclic heterocyclyl represented by R³ are each optionally substituted by one to three R⁹; each R⁹ is independently —OR^{O1}, —NR^{N1}R^{N2}, 5 to 7 membered monocyclic heterocyclyl, or C₁₋₄ alkyl; each R^{O1} is independently H, C₁₋₄ alkyl or C₃₋₅ cycloalkyl, wherein the C₁₋₄ alkyl and C₃₋₅ cycloalkyl are each optionally substituted by R^{O2}; each R^{O2} is independently —CN or C₁₋₄ alkoxy; and R^{N1} and R^{N2} are each independently H or C₁₋₃ alkyl. In some embodiments, R³ is a 5 to 7 membered monocyclic or bicyclic heterocyclyl linked to ring B through a ring N atom. In some embodiments, each R⁹ is independently —OR^{O1}, —NR^{N1}R^{N2}, or C₁₋₄ alkyl.

In a forty-eighth embodiment, for compounds of formula (VII), (VII′), (VIII) or (VIII′), or pharmaceutically acceptable salts thereof, ring C is pyridinyl, pyrazinyl or pyrimidinyl, each of which is optionally substituted by one or two R^{C}; and the remaining variables are as described in the first aspect or the forty-seventh embodiment or any alternative embodiments described therein.

In a forty-ninth embodiment, for compounds of formula (VII), (VII′), (VIII) or (VIII′), or pharmaceutically acceptable salts thereof, ring C is pyrimidinyl, pyrazinyl or thiazolyl, each of which is optionally substituted by one or two R^{C}; and the remaining variables are as described in the first aspect or the forty-seventh embodiment or any alternative embodiments described therein.

In a fiftieth embodiment, for compounds of formula (VII), (VII′), (VIII) or (VIII′), or pharmaceutically acceptable salts thereof, ring C is selected from:

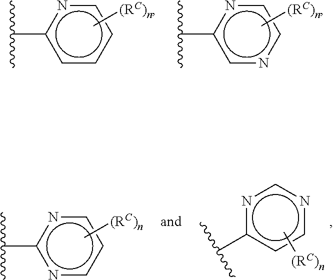

and wherein

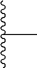

represents a bond to ring B, and n is 0, 1, or 2; and the remaining variables are as described in the first aspect or the forty-seventh embodiment or any alternative embodiments described therein.

In a fifty-first embodiment, for compounds of formula (VII), (VII′), (VIII) or (VIII′), or pharmaceutically acceptable salts thereof, ring C is selected from:

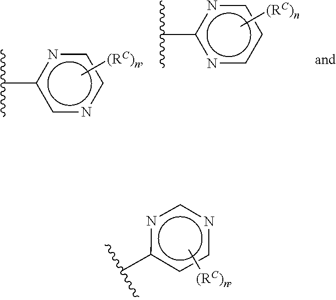

and wherein

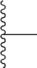

represents a bond to ring B, and n is 0, 1, or 2; and the remaining variables are as described in the first aspect or the forty-seventh embodiment or any alternative embodiments described therein.

In a fifty-second embodiment, for compounds of formula (VII), (VII′), (VIII) or (VIII′), or pharmaceutically acceptable salts thereof, ring C is selected from:

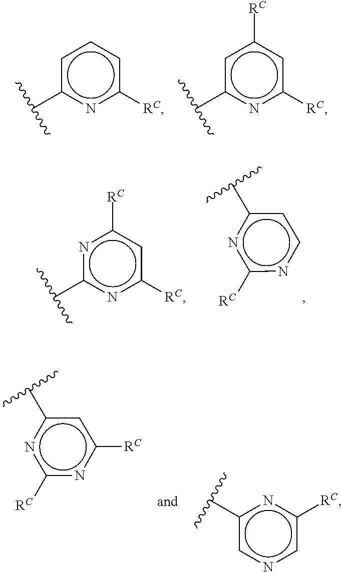

and wherein

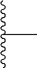

represents a bond to ring B, and two R^{C} groups in ring C may be the same or different; and the remaining variables are as described in the first aspect or the forty-seventh embodiment or any alternative embodiments described therein.

In a fifty-third embodiment, for compounds of formula (VII) or (VIII), or pharmaceutically acceptable salts thereof, ring C is selected from:

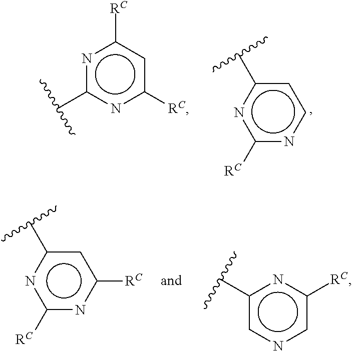

and wherein

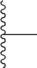

represents a bond to ring B, and two R^{C} groups in ring C may be the same or different; and the remaining variables are as described in the first aspect or the forty-seventh embodiment.

In a fifty-fourth embodiment, for compounds of formula (VII) or (VIII), or pharmaceutically acceptable salts thereof, each R^{C} is independently —ORO1, C1-2alkyl, C1-2haloalkyl, or 5 membered oxygen-containing heterocyclyl optionally substituted with one RC1; and the remaining variables are as described in the first aspect or the forty-seventh, forty-eighth, forty-ninth, fiftieth, fifty-first, fifty-second or fifty-third embodiment. In some embodiments, at least one of R^{C} is C₁₋₂haloalkyl, more specifically —CF₂CH₃.

In a fifty-fifth embodiment, for compounds of formula (VII), (VII′), (VIII) or (VIII′), or pharmaceutically acceptable salts thereof, R^{C} is tetrahydrofuranyl optionally substituted with one R^{C1}; and the remaining variables are as described in the first aspect or the forty-seventh, forty-eighth, forty-ninth, fiftieth, fifty-first, fifty-second or fifty-third embodiment or any alternative embodiments described therein.

In a fifty-sixth embodiment, for compounds of formula (VII), (VII′), (VIII) or (VIII′), or pharmaceutically acceptable salts thereof, R^{C} is

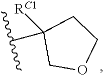

wherein

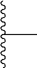

represents a bond ring C; and the remaining variables are as described in the first aspect or the forty-seventh, forty-eighth, forty-ninth, fiftieth, fifty-first, fifty-second or fifty-third embodiment or any alternative embodiments described therein or any alternative embodiments described therein.

In a fifty-seventh embodiment, for compounds of formula (VII), (VII′), (VIII) or (VIII′), or pharmaceutically acceptable salts thereof, each R^{C1} is independently F or —OCH₃; and the remaining variables are as described in the first aspect or the forty-seventh, forty-eighth, forty-ninth, fiftieth, fifty-first, fifty-second, fifty-thirty, fifty-fourth, fifty-fifth or fifty-sixth embodiment or any alternative embodiments described therein or any alternative embodiments described therein.

In a fifty-eighth embodiment, for compounds of formula (VII), (VII′), (VIII) or (VIII′), or pharmaceutically acceptable salts thereof, each R^{C} is independently selected from —CH₃, —CF₂CH₃, —OCH₃, —O—CH₂—CH₂—O—CH₃,

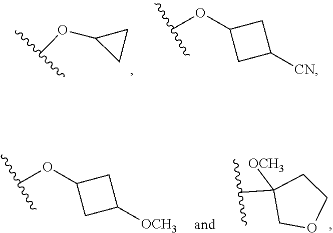

wherein

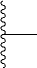

represents a bond to ring C; and the remaining variables are as described in the first aspect or the forty-seventh, forty-eighth, forty-ninth, fiftieth or fifty-first, fifty-second, fifty-thirty, fifty-fourth, fifty-fifth or fifty-sixth embodiment or any alternative embodiments described therein. In some embodiments, each R^{C} is independently selected from —CH₃, —CH₂CH₃, —CF₂CH₃, —CF(CH₃)₂ —OCH₃, —O—CH₂—CH₂—O—CH₃, CN OCH₃

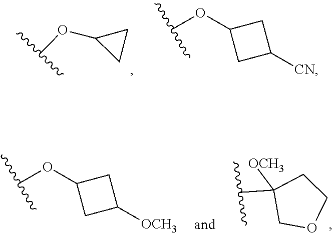

wherein

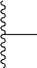

represents a bond to ring C. In an alternative fifty-eighth embodiment, for compounds of formula (VII), (VII′), (VIII) or (VIII′), or pharmaceutically acceptable salts thereof, each R^{C} is independently selected from —CH₃, —CH₂CH₃, —CF₂CH₃, —CF(CH₃)₂, —OCH₃, —O—CH₂—CH₂—O—CH₃,

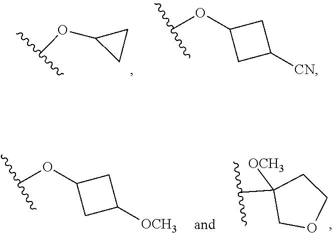

wherein

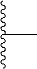

represents a bond to ring C; and the remaining variables are as described in the first aspect or the forty-seventh, forty-eighth, forty-ninth, fiftieth or fifty-first, fifty-second, fifty-thirty, fifty-fourth, fifty-fifth or fifty-sixth embodiment or any alternative embodiments described therein.

In a fifty-ninth embodiment, for compounds of formula (VII), (VII′), (VIII) or (VIII′), or pharmaceutically acceptable salts thereof, the 5 to 7 membered monocyclic or bicyclic heterocyclyl represented by R³ is selected from 3,6-diazabicyclo[3.1.1]heptanyl, 2-oxa-5-azabicylo [2.2.1]heptanyl, 6-oxa-3-azabicyclo [3.1.1]heptanyl, piperazinyl, and pyrrolidinyl, each of which is optionally substituted by one or two R⁹; and the remaining variables are as described in the first aspect or the forty-seventh, forty-eighth, forty-ninth, fiftieth, fifty-first, fifty-second, fifty-thirty, fifty-fourth, fifty-fifth, fifty-sixth, fifty-seventh or fifty-eighth embodiment or any alternative embodiments described therein.

In a sixtieth embodiment, for compounds of formula (VII), (VII′), (VIII) or (VIII′), or pharmaceutically acceptable salts thereof, R³ is selected from H, —CH₂CH₃,

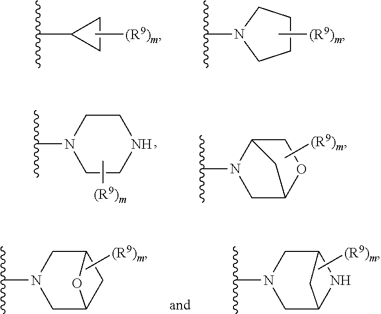

wherein

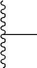

represents a bond to ring B, and m is 0, 1 or 2; and the remaining variables are as described in the first aspect or the forty-seventh, forty-eighth, forty-ninth, fiftieth, fifty-first, fifty-second, fifty-thirty, fifty-fourth, fifty-fifth, fifty-sixth, fifty-seventh or fifty-eighth embodiment or any alternative embodiments described therein.

In a sixty-first embodiment, for compounds of formula (VII), (VII′), (VIII) or (VIII′), or pharmaceutically acceptable salts thereof, R³ is selected from H, —CH₂CH₃,

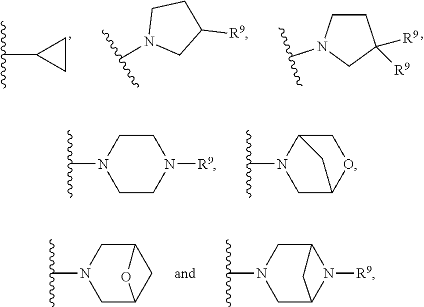

wherein

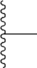

represents a bond to ring B; and the remaining variables are as described in the first aspect or the forty-seventh, forty-eighth, forty-ninth, fiftieth, fifty-first, fifty-second, fifty-thirty, fifty-fourth or fifty-fifth, fifty-sixth, fifty-seventh or fifty-eighth embodiment or any alternative embodiments described therein.

In a sixty-second embodiment, for compounds of formula (VII), (VII′), (VIII) or (VIII′), or pharmaceutically acceptable salts thereof, each R⁹ is independently selected —OH, —OCH₃, —N(CH₃)₂ and —CH₃; and the remaining variables are as described in the first aspect or the forty-seventh, forty-eighth, forty-ninth, fiftieth, fifty-first, fifty-second, fifty-thirty, fifty-fourth, fifty-fifth, fifty-sixth, fifty-seventh or fifty-eighth embodiment or any alternative embodiments described therein. In some embodiments, each R⁹ is independently selected from —OH, —OCH₃, —NH₂, —NHCH₃, —N(CH₃)₂, —N(CH₃)(CH₂CH₃), —N(CH₂CH₃)₂, pyrrolidinyl, morpholinyl, —CH₂CH₃, and —CH₃.

In a sixty-third embodiment, for compounds of formula (VII), (VII′), (VIII) or (VIII′), or pharmaceutically acceptable salts thereof, R^{O1} are each independently selected from H, —CH₃, —CH₂CH₂OCH₃, cyclopropyl,

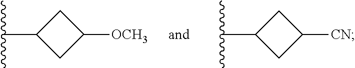 [SMILES: *C1CC(OC)C1 ; *C1CC(C#N)C1]

and the remaining variables are as described in the first aspect or the forty-seventh, forty-eighth, forty-ninth, fiftieth, fifty-first, fifty-second, fifty-thirty, fifty-fourth, fifty-fifth, fifty-sixth, fifty-seventh, fifty-eighth, fifty-ninth, sixtieth, sixty-first or sixty-second embodiment or any alternative embodiments described therein.

In a sixty-fourth embodiment, for compounds of formula (VII), (VII′), (VIII) or (VIII′), or pharmaceutically acceptable salts thereof, ring C is selected from the following:

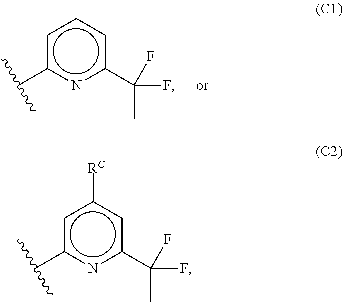

wherein R^{C} is C₁₋₃alkyl or C₁₋₃alkoxy; and the remaining variables are as defined in the fifty-ninth, sixtieth, sixty-first or sixty-second embodiment or any alternative embodiments described therein. In some embodiments, R^{C} is C₁₋₂alkyl or C₁₋₂alkoxy.

In a sixty-fifth embodiment, for compounds of formula (VII), (VII′), (VIII) or (VIII′), or pharmaceutically acceptable salts thereof, ring C is selected from:

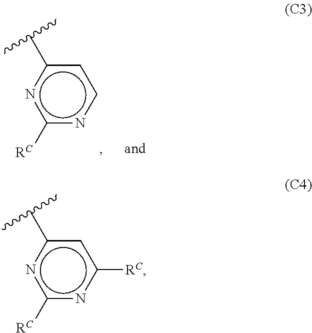

wherein R^{C}, for each occurrence, is independently C₁₋₃alkyl, C₁₋₃haloalkyl or C₁₋₃alkoxy; and the remaining variables are as defined in the fifty-ninth, sixtieth, sixty-first or sixty-second embodiment or any alternative embodiments described therein. In some embodiments, for formula (C3), R^{C} is C₁₋₃haloalkyl; for formula (C4), one of R^{C} is C₁₋₃alkyl, and the other is C₁₋₃alkyl or C₁₋₃alkoxy. In some embodiments, for formula (C3), R^{C} is —CF₂CH₃; for formula (C4), one of R^{C} is —CF₂CH₃, and the other is C₁₋₃alkyl or C₁₋₃alkoxy.

In a sixty-sixth embodiment, for compounds of the sixty-fourth or sixty-fifth embodiment, or pharmaceutically acceptable salts thereof, R³ is selected from pyrrolidinyl, morphonlinyl, 6-oxa-3-azabicyclo[3.1.1]heptanyl, and 3,6-diazabicyclo[3.1.1]heptanyl, each of which is optionally substituted by one or two R⁹; R⁹ is independently halo, C₁₋₄ alkyl, OH, —OC₁₋₄alkyl, or —NR^{a2}R^{a3}; and R^{a2} and R^{a3} are each independently H or C₁₋₃alkyl. In some embodiments, R³ is selected from:

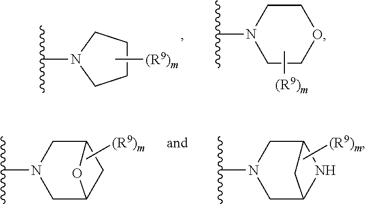

wherein R⁹ is C₁₋₃alkyl, —OC₁₋₃alkyl or —NR^{a2}R^{a3}; and m is 0, 1 or 2.

In a sixty-seventh embodiment, for compounds of the sixty-fourth, sixty-fifth or sixty-sixth embodiment, or a pharmaceutically acceptable salts thereof, when only one R^{C} is present, R^{C} is —CF₂CH₃, or when two R^{C} are present, one of R^{C} is —CF₂CH₃ and the other R^{C} is C₁₋₃alkyl or C₁₋₃alkoxy; m is 1 or 2; and R⁹ for each occurrence is independently C₁₋₃alkyl, —OC₁₋₃alkyl or —NR^{a2}R^{a3}; and the remaining variables as defined in the sixty-fourth, sixty-fifth or sixty-sixth embodiment.

In a sixty-eighth embodiment, for compounds of the sixty-fourth or sixty-fifth embodiment, or a pharmaceutically acceptable salts thereof, ring C is

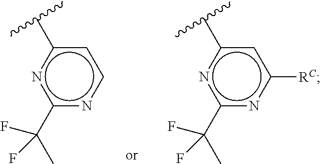

R^{C} is C₁₋₂alkyl or C₁₋₂alkoxy; R³ is

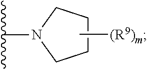

m is 1 or 2; R⁹ for each occurrence, is independently C₁₋₂alkyl and —NR^{a2}R^{a3}; and R^{a2} and R^{a3} are C₁₋₂alkyl. In some embodiments, R^{C} is —CH₃, —CH₂CH₃ or —OCH₃; and R⁹ for each occurrence is independently —CH₃ or —N(CH₃)₂.

In a sixty-ninth embodiment, for compounds of the sixty-fourth or sixty-fifth embodiment, or a pharmaceutically acceptable salts thereof, when only one R^{C} is present, R^{C} is —CF₂CH₃, or when two R^{C} are present, one of R^{C} is —CF₂CH₃ and the other R^{C} is —CH₃, —CH₂CH₃ or —OCH₃; and R⁹ for each occurrence is independently —CH₃ or —N(CH₃)₂

In a seventieth embodiment, the compound is represented by formula (VIIA):

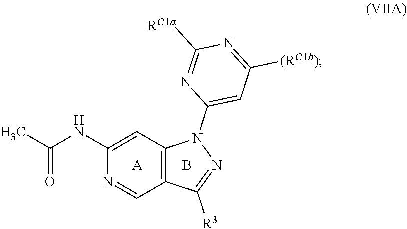

or a pharmaceutically acceptable salt thereof, wherein R^{C1a} is C₁₋₃alkyl substituted with 1 to 3 halo; R^{C1b} is C₁₋₃alkyl; R³ is selected from

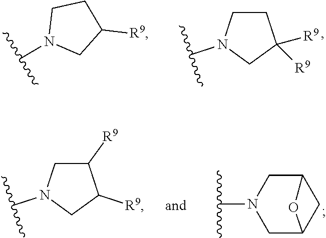

each R⁹ is independently —NR^{N1}R^{N2}, C₁₋₄ alkyl, morpholinyl, or pyrrolidinyl; and R^{N1} and R^{N2} are each independently H or C₁₋₃ alkyl. In an alternative seventieth embodiment, the compound is represented by formula (VIIA′):

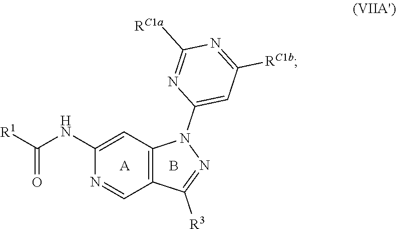

or a pharmaceutically acceptable salt thereof, wherein R₁ is H or CH₃; R^{C1a} is C₁₋₃ alkyl substituted with 1 to 3 halo; R^{C1b} is C₁₋₃alkyl; R³ is selected from

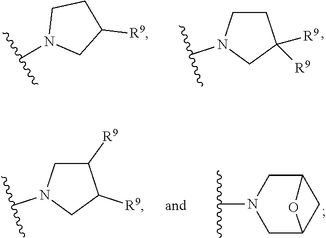

each R⁹ is independently —NR^{N1}R^{N2}, C₁₋₄ alkyl, morpholinyl, or pyrrolidinyl; and R^{N1} and R^{N2} are each independently H or C₁₋₃ alkyl.

In a seventy-first embodiment, for compounds of the seventieth embodiment, or a pharmaceutically acceptable salts thereof, R^{C1a} is —CF₂CH₃; R^{C1b} is —CH₃ or CH₂CH₃; and the remaining variables are as described in the first aspect or the seventieth embodiment.

In a seventy-second embodiment, for compounds of the seventieth or seventy-first embodiment, or a pharmaceutically acceptable salts thereof, each R⁹ is independently —NH₂, —NHCH₃, —N(CH₃)₂, —N(CH₃)(CH₂CH₃), —N(CH₂CH₃)₂,

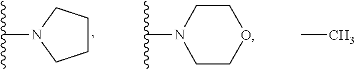

or —CH₂CH₃; and the remaining variables are as described in the first aspect or the seventieth or seventy-first embodiment.

In a seventy-third embodiment, the compound is represented by formula (VIIB):

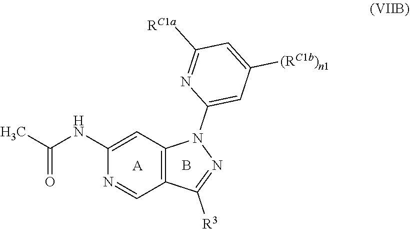

or a pharmaceutically acceptable salt thereof, wherein: R^{C1a} is C₁₋₃alkyl substituted with 1 to 3 halo; R^{C1b} is C₁₋₃alkoxy; n1 is 0 or 1; R³ is

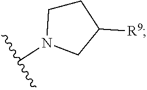

R⁹ is —NR^{N1}R^{N2}; and R^{N1} and R^{N2} are each independently H or C₁₋₃ alkyl.

In a seventy-fourth embodiment, for compounds of the seventy-third embodiment, or a pharmaceutically acceptable salts thereof, R^{C1a} is —CF₂CH₃ or —CF(CH₃)₂; and the remaining variables are as described in the first aspect or the seventy-third embodiment.

In a seventy-fifth embodiment, for compounds of the seventy-third or seventy-fourth embodiment, or a pharmaceutically acceptable salts thereof, n1 is 0 or n1 is 1 and R^{C1b} is —OCH₃; and the remaining variables are as described in the first aspect or the seventy-third or seventy-fourth embodiment.

In a seventy-sixth embodiment, for compounds of the seventieth, seventy-first, seventy-second, seventy-third, seventy-fourth, or seventy-fifth embodiment, or a pharmaceutically acceptable salts thereof, R⁹ is —NHCH(CH₃)₂; and the remaining variables are as described in the first aspect or the seventieth, seventy-first, seventy-second, seventy-third, seventy-fourth, or seventy-fifth embodiment.

In some embodiments, for compounds of any one of the forty-seventh to sixty-ninth embodiments, the compound is represented by formula (VII) or a pharmaceutically acceptable salt thereof, and the definitions for variables depicted therein are as defined in any one of the forty-seventh to sixty-ninth embodiments. In some embodiments, for compounds of any one of the sixty-fourth to sixty-ninth embodiments, the compound is represented by formula (VIII) or a pharmaceutically acceptable salt thereof, and the definitions for variables depicted therein are as defined in any one of the forty-seventh to sixty-ninth embodiments.

In one embodiment, the compound of present disclosure is any one of compounds of Examples 1-958 or a pharmaceutically acceptable salt thereof.

The disclosure also includes both the neutral form and pharmaceutically acceptable salts of the compounds illustrated in the exemplification.

### III. Pharmaceutical Compositions

In one aspect, the present disclosure is a pharmaceutical composition comprising at least one compound described herein, or a pharmaceutically acceptable salt thereof, and at least one pharmaceutically acceptable carrier.

The phrase “pharmaceutically acceptable” indicates that the substance, composition or dosage form must be compatible chemically and/or toxicologically, with the other ingredients comprising a formulation, and/or the mammal being treated therewith.

As used herein, the term “pharmaceutically acceptable carrier” includes generally recognized as safe (GRAS) solvents, dispersion media, surfactants, antioxidants, preservatives (e.g., antibacterial agents, antifungal agents), isotonic agents, salts, preservatives, drug stabilizers, buffering agents (e.g., maleic acid, tartaric acid, lactic acid, citric acid, acetic acid, sodium bicarbonate, sodium phosphate, and the like), and the like and combinations thereof, as would be known to those skilled in the art (see, for example, Remington's Pharmaceutical Sciences, 18th Ed. Mack Printing Company, 1990, pp. 1289-1329). Except insofar as any conventional carrier is incompatible with the active ingredient, its use in the therapeutic or pharmaceutical compositions is contemplated.

The formulations may be prepared using conventional dissolution and mixing procedures. For example, the bulk drug substance (i.e., compound of the present disclosure or stabilized form of the compound (e.g., complex with a cyclodextrin derivative or other known complexation agent)) is dissolved in a suitable solvent in the presence of one or more of the excipients described above. The compound of the present disclosure is typically formulated into pharmaceutical dosage forms to provide an easily controllable dosage of the drug and to give the patient an elegant and easily handleable product.

The pharmaceutical composition (or formulation) for application may be packaged in a variety of ways depending upon the method used for administering the drug. Generally, an article for distribution includes a container having deposited therein the pharmaceutical formulation in an appropriate form. Suitable containers are well-known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

The pharmaceutical composition comprising a compound of the present disclosure is generally formulated for use as a parenteral or oral administration.

For example, the pharmaceutical oral compositions of the present disclosure can be made up in a solid form (including without limitation capsules, tablets, pills, granules, powders or suppositories), or in a liquid form (including without limitation solutions, suspensions or emulsions). The pharmaceutical compositions can be subjected to conventional pharmaceutical operations such as sterilization and/or can contain conventional inert diluents, lubricating agents, or buffering agents, as well as adjuvants, such as preservatives, stabilizers, wetting agents, emulsifiers and buffers, etc.

Typically, the pharmaceutical compositions are tablets or gelatin capsules comprising the active ingredient together with a) diluents, e.g., lactose, dextrose, sucrose, mannitol, sorbitol, cellulose and/or glycine; b) lubricants, e.g., silica, talcum, stearic acid, its magnesium or calcium salt and/or polyethyleneglycol; for tablets also c) binders, e.g., magnesium aluminum silicate, starch paste, gelatin, tragacanth, methylcellulose, sodium carboxymethylcellulose and/or polyvinylpyrrolidone; if desired d) disintegrants, e.g., starches, agar, alginic acid or its sodium salt, or effervescent mixtures; and/or e) absorbents, colorants, flavors and sweeteners.

Tablets may be either film coated or enteric coated according to methods known in the art.

Suitable compositions for oral administration include a compound of the disclosure in the form of tablets, lozenges, aqueous or oily suspensions, dispersible powders or granules, emulsion, hard or soft capsules, or syrups or elixirs. Compositions intended for oral use are prepared according to any method known in the art for the manufacture of pharmaceutical compositions and such compositions can contain one or more agents selected from the group consisting of sweetening agents, flavoring agents, coloring agents and preserving agents in order to provide pharmaceutically elegant and palatable preparations. Tablets may contain the active ingredient in admixture with nontoxic pharmaceutically acceptable excipients which are suitable for the manufacture of tablets. These excipients are, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets are uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

The parenteral compositions (e.g, intravenous (IV) formulation) are aqueous isotonic solutions or suspensions. The parenteral compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. The compositions are generally prepared according to conventional mixing, granulating or coating methods, respectively, and contain about 0.1-75%, or contain about 1-50%, of the active ingredient.

The effective dose of a compound provided herein, or a pharmaceutically acceptable salt thereof, administered to a subject can be 10 μg-500 mg.

Administering a compound described herein, or a pharmaceutically acceptable salt thereof, to a mammal comprises any suitable delivery method. Administering a compound described herein, or a pharmaceutically acceptable salt thereof, to a mammal includes administering a compound described herein, or a pharmaceutically acceptable salt thereof, topically, enterally, parenterally, transdermally, transmucosally, via inhalation, intracisternally, epidurally, intravaginally, intravenously, intramuscularly, subcutaneously, intradermally or intravitreally to the mammal. Administering a compound described herein, or a pharmaceutically acceptable salt thereof, to a mammal also includes administering topically, enterally, parenterally, transdermally, transmucosally, via inhalation, intracisternally, epidurally, intravaginally, intravenously, intramuscularly, subcutaneously, intradermally or intravitreally to a mammal a compound that metabolizes within or on a surface of the body of the mammal to a compound described herein, or a pharmaceutically acceptable salt thereof.

Thus, a compound or pharmaceutically acceptable salt thereof as described herein, may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the compound or pharmaceutically acceptable salt thereof as described herein may be combined with one or more excipients and used in the form of ingestible tablets, buccal tablets, troches, capsules, elixirs, suspensions, syrups, or wafers, and the like. Such compositions and preparations should contain at least about 0.1% of active compound. The percentage of the compositions and preparations may, of course, be varied and may conveniently be between about 2 to about 60% of the weight of a given unit dosage form. The amount of active compound in such therapeutically useful compositions can be such that an effective dosage level will be obtained.

The tablets, troches, pills, capsules, and the like can include the following: binders such as gum tragacanth, acacia, corn starch or gelatin; excipients such as dicalcium phosphate; a disintegrating agent such as corn starch, potato starch, alginic acid and the like; a lubricant such as magnesium stearate; or a sweetening agent such as sucrose, fructose, lactose or aspartame or a flavoring agent.

The active compound may also be administered intravenously or intraperitoneally by infusion or injection. Solutions of the active compound or its salts can be prepared in water, optionally mixed with a nontoxic surfactant.

Exemplary pharmaceutical dosage forms for injection or infusion can include sterile aqueous solutions or dispersions or sterile powders comprising the active ingredient which are adapted for the extemporaneous preparation of sterile injectable or infusible solutions or dispersions. In all cases, the ultimate dosage form should be sterile, fluid and stable under the conditions of manufacture and storage.

Sterile injectable solutions can be prepared by incorporating the active compound in the required amount in the appropriate solvent with various of the other ingredients enumerated above, as required, followed by filter sterilization. In the case of sterile powders for the preparation of sterile injectable solutions, the preferred methods of preparation can be vacuum drying and the freeze drying techniques, which can yield a powder of the active ingredient plus any additional desired ingredient present in the previously sterile-filtered solutions.

Exemplary solid carriers can include finely divided solids such as talc, clay, microcrystalline cellulose, silica, alumina and the like. Useful liquid carriers include water, alcohols or glycols or water-alcohol/glycol blends, in which the compounds or pharmaceutically acceptable salts thereof as described herein can be dissolved or dispersed at effective levels, optionally with the aid of non-toxic surfactants.

Useful dosages of a compound or pharmaceutically acceptable salt thereof as described herein can be determined by comparing their in vitro activity, and in vivo activity in animal models. Methods for the extrapolation of effective dosages in mice, and other animals, to humans are known to the art; for example, see U.S. Pat. No. 4,938,949, which is incorporated by reference in its entirety.

The amount of a compound or pharmaceutically acceptable salt thereof as described herein, required for use in treatment can vary not only with the particular salt selected but also with the route of administration, the nature of the condition being treated and the age and condition of the patient and can be ultimately at the discretion of the attendant physician or clinician. In general, however, a dose can be in the range of from about 0.1 to about 10 mg/kg of body weight per day.

The compound or pharmaceutically acceptable salt thereof as described herein can be conveniently administered in unit dosage form; for example, containing 0.01 to 10 mg, or 0.05 to 1 mg, of active ingredient per unit dosage form. In some embodiments, a dose of 5 mg/kg or less can be suitable.

The desired dose may conveniently be presented in a single dose or as divided doses administered at appropriate intervals.

### IV. Use of Compounds and Compositions of the Disclosure

The compounds, or pharmaceutically acceptable salts thereof described herein may be used to decrease or inhibit the activity of TYK2 or to otherwise affect the properties and/or behavior of TYK2, e.g., stability, phosphorylation, kinase activity, interactions with other proteins, etc.

Another aspect of the present disclosure is a method of inhibiting TYK2 activity in a subject in need thereof comprising administering to the subject an effective amount of at least one compound described herein, or a pharmaceutically acceptable salt thereof, or a pharmaceutical composition described herein.

In some aspect, the present disclosure is a method of treating a disease or disorder responsive to inhibition of TYK2 in a subject comprising administering to the subject an effective amount of at least one compound described herein, or a pharmaceutically acceptable salt thereof, or a pharmaceutical composition described herein.

The present disclosure also includes the use of at least one compound described herein, or a pharmaceutically acceptable salt thereof, or a pharmaceutically composition described herein for the manufacture of a medicament for inhibiting TYK2 activity. Also included is the use of at least one compound described herein, or a pharmaceutically acceptable salt thereof, or a pharmaceutically composition described herein for the manufacture of a medicament for treating a disease or disorder responsive to inhibition of TYK2.

The disclosure also provides a compound described herein, or a pharmaceutically acceptable salt thereof, or a pharmaceutical composition described herein for use in inhibiting TYK2 activity. Also provided is a compound described herein, or a pharmaceutically acceptable salt thereof, or a pharmaceutical composition described herein for use in treating a disease or disorder responsive to inhibition of TYK2.

As used herein, the term “inhibit”, “inhibition” or “inhibiting” refers to the reduction or suppression of a given condition, symptom, or disorder, or disease, or a significant decrease in the baseline activity of a biological activity or process.

As used herein, the term “subject” and “patient” may be used interchangeably, and means a mammal in need of treatment, e.g., human, companion animals (e.g., dogs, cats, and the like), farm animals (e.g., cows, pigs, horses, sheep, goats and the like) and laboratory animals (e.g., rats, mice, guinea pigs and the like). Typically, the subject is a human in need of treatment.

As used herein, a subject is “in need of” a treatment if such subject would benefit biologically, medically or in quality of life from such treatment (preferably, a human).

As used herein, the term “treat”, “treating” or “treatment” refers to obtaining desired pharmacological and/or physiological effect. The effect can be therapeutic, which includes achieving, partially or substantially, one or more of the following results: partially or totally reducing the extent of the disease, disorder or syndrome; ameliorating or improving a clinical symptom or indicator associated with the disorder; or delaying, inhibiting or decreasing the likelihood of the progression of the disease, disorder or syndrome.

As used herein the term “co-administer” refers to the presence of two active agents in the blood of an individual. Active agents that are co-administered can be concurrently or sequentially delivered.

The term “combination therapy” or “in combination with” or “pharmaceutical combination” refers to the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients. Alternatively, such administration encompasses co-administration in multiple, or in separate containers (e.g., capsules, powders, and liquids) for each active ingredient. Powders and/or liquids may be reconstituted or diluted to a desired dose prior to administration. In addition, such administration also encompasses use of each type of therapeutic agent being administered prior to, concurrent with, or sequentially to each other with no specific time limits. In each case, the treatment regimen will provide beneficial effects of the drug combination in treating the conditions or disorders described herein.

In some embodiment, the method described herein treats the disease or disorder responsive to inhibition of TYK2, wherein the disease or disorder includes inflammation, autoimmune disease, neuroinflammation, arthritis, rheumatoid arthritis, spondyloarthropathies, systemic lupus erythematous, lupus nephritis, arthritis, osteoarthritis, gouty arthritis, pain, fever, pulmonary sarcoisosis, silicosis, cardiovascular disease, atherosclerosis, myocardial infarction, thrombosis, congestive heart failure and cardiac reperfusion injury, cardiomyopathy, stroke, ischaemia, reperfusion injury, brain edema, brain trauma, neurodegeneration, liver disease, inflammatory bowel disease, Crohn's disease, ulcerative colitis, nephritis, retinitis, retinopathy, macular degeneration, glaucoma, diabetes (type 1 and type 2), diabetic neuropathy, viral and bacterial infection, myalgia, endotoxic shock, toxic shock syndrome, autoimmune disease, osteoporosis, multiple sclerosis, endometriosis, menstrual cramps, vaginitis, candidiasis, cancer, fibrosis, obesity, muscular dystrophy, polymyositis, dermatomyositis, autoimmune hepatitis, primary biliary cirrhosis, primary sclerosing cholangitis, vitiligo, alopecia, Alzheimer's disease, skin flushing, eczema, psoriasis, atopic dermatitis and sunburn.

The term “autoimmune disorders” includes diseases or disorders involving inappropriate immune response against native antigens, such as acute disseminated encephalomyelitis (ADEM), Addison's disease, alopecia areata, antiphospholipid antibody syndrome (APS), autoimmune hemolytic anemia, autoimmune hepatitis, bullous pemphigoid (BP), Coeliac disease, dermatomyositis, diabetes mellitus type 1, Goodpasture's syndrome, Graves' disease, Guillain-Barre syndrome (GBS), Hashimoto's disease, idiopathic thrombocytopenic purpura, lupus erythematosus, mixed connective tissue disease, multiple sclerosis, myasthenia gravis, pemphigus vulgaris, pernicious anaemia, polymyositis, primary biliary cirrhosis, Sjogren's syndrome, temporal arteritis, and Wegener's granulomatosis.

The term “inflammatory disorders” includes diseases or disorders involving acute or chronic inflammation such as allergies, asthma, atopic dermatitis, prostatitis, glomerulonephritis, pelvic inflammatory disease (PID), inflammatory bowel disease (IBD, e.g., Crohn's disease, ulcerative colitis), reperfusion injury, rheumatoid arthritis, transplant rejection, and vasculitis.

The term “cancer” includes diseases or disorders involving abnormal cell growth and/or proliferation, such as glioma, thyroid carcinoma, breast carcinoma, lung cancer (e.g. small-cell lung carcinoma, non-small-cell lung carcinoma), gastric carcinoma, gastrointestinal stromal tumors, pancreatic carcinoma, bile duct carcinoma, ovarian carcinoma, endometrial carcinoma, prostate carcinoma, renal cell carcinoma, lymphoma (e.g., anaplastic large-cell lymphoma), leukemia (e.g. acute myeloid leukemia, T-cell leukemia, chronic lymphocytic leukemia), multiple myeloma, malignant mesothelioma, malignant melanoma, and colon cancer (e.g. microsatellite instability-high colorectal cancer).

The disclosed method can include a kit comprising a compound or pharmaceutically acceptable salt thereof as described herein and instructional material which can describe administering a compound or pharmaceutically acceptable salt thereof as described herein or a composition comprising a compound or pharmaceutically acceptable salt thereof as described herein to a cell or a subject. This should be construed to include other embodiments of kits that are known to those skilled in the art, such as a kit comprising a (such as sterile) solvent for dissolving or suspending a compound or pharmaceutically acceptable salt thereof as described herein or composition prior to administering a compound or pharmaceutically acceptable salt thereof as described herein or composition to a cell or a subject. In some embodiments, the subject can be a human.

### EXEMPLIFICATIONS

For illustrative purposes, the syntheses described below provide routes for synthesizing the compounds of the present disclosure as well as key intermediates. Although specific starting materials and reagents are illustrated in the synthetic protocols below, other starting materials and reagents can be substituted to provide a variety of derivatives and/or reaction conditions. In addition, many of the compounds prepared by the procedures described below can be further modified in light of this disclosure using conventional chemistry well known to those skilled in the art.

### Abbreviations

Aq. means aqueous;
Bn means benzyl;
Boc means tert-butoxy carbonyl;
Boc₂O means di-tert-butyl dicarbonate
br means broad;
t-BuOH means tertiary butanol
n-BuLi means n-butyl lithium;
d means doublet;
dd means double doublet;
DCM means dichloromethane;
DEA means diethylamine
DIPEA means N-ethyldiisopropylamine or N,N-diisopropylethylamine;
DMA means N,N-dimethylacetamide;
DMF means N,N-dimethylformamide;
DMSO means Dimethylsulfoxide;
DMSO-d₆ means hexadeuterodimethyl sulfoxide;
Et means ethyl;
EtOH means ethanol;
EtOAc means ethyl acetate;
Eq. means equivalent;
HPLC means high pressure liquid chromatography;
IPA means 2-propanol
LCMS means liquid chromatography mass spectrometry;
LDA means lithium diisopropylamide;
m means multiplet;
Me means methyl;
MeCN means acetonitrile;
MeI means iodomethane
MeOH means methanol;
MeOH-d₄ means deutero-methanol;
MS m/z means mass spectrum peak;
MsCl means methanesulfonyl chloride;
NBS means N-bromosuccinimide;
NOE means nuclear Overhauser effect spectroscopy;
PE means petroleum ether;
Pd(amphos)Cl₂ means bis(di-tert-butyl(4-dimethylaminophenyl)phosphine) dichloropalladium(II)
Pd₂(dba)₃ means tris(dibenzylideneacetone)dipalladium (0);
Pd(dppf)Cl₂ means [1,1′-bis(diphenylphosphino)ferrocene]dichloropalladium(II);
PdCl₂(PPh₃)₂ means bis(triphenylphosphine)palladium(II) dichloride;
Pd/C means palladium on charcoal;
q means quartet;
rt means room temperature;
s means singlet;
sat. means saturated;
SFC means supercritical fluid chromatography;
soln. means solution;
t means triplet;
TBME means tert-butyl methyl ether;
TEA means triethylamine;
TFA means trifluoroacetic acid;
THF means tetrahydrofuran;
TLC means thin layer chromatography;
TsCl means para-toluenesulfonyl chloride;
TsOH means para-toluenesulfonic acid; and
RuPhos Pd G3 means (2-dicyclohexylphosphino-2′,6′-diisopropoxy-1,1′-biphenyl)[2-(2′-amino-1,1′-biphenyl)]palladium(II) methanesulfonate.

According to a first process, compounds of Formula (I), may be prepared from compounds of Formulae (II) and (III), as shown in Scheme 1

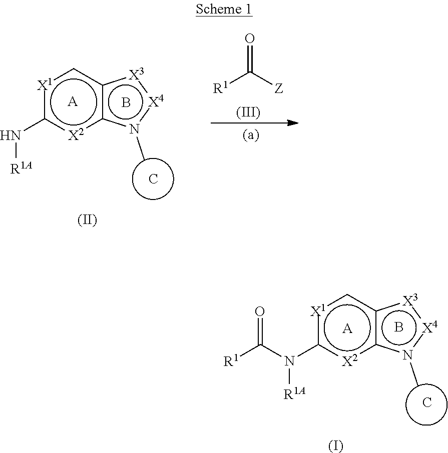

Z is Cl or OC(O)R¹.

The compound of Formula (I) may be prepared from the amine of Formula (II) and the compound of Formula (III) according to process step (a) an amidation reaction, in the presence of a suitable organic base, optionally in a suitable polar aprotic solvent, at elevated temperature. Preferred conditions, comprise reaction of the compound of Formula (II) with the compound of Formula (III) in the presence of TEA or DIPEA, optionally in DMF at between 25 and 70° C.

According to a second process, compounds of Formula (I), may be prepared from compounds of Formulae (IV) and (V), as shown in Scheme 2

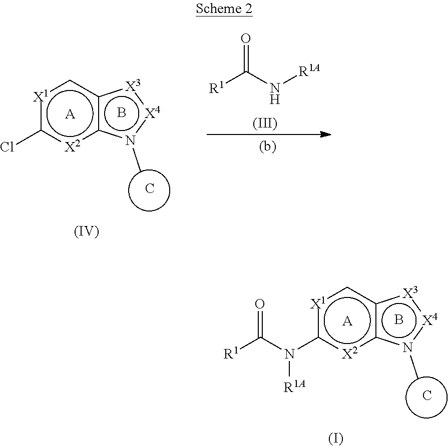

The compound of Formula (I) may be prepared from the compounds of Formulae (IV) and (V) according to process step (b) a Buchwald-Hartwig cross coupling reaction. Typical conditions comprise, reaction of the amide of Formula (V) with the chloride of Formula (IV) in the presence of a suitable inorganic base, a suitable palladium catalyst in the presence of suitable phosphine ligands, in a suitable solvent at elevated temperature, optionally under microwave irradiation. Preferred conditions comprise, reaction of the compounds of Formulae (IV) and (V) in the presence of Brettphos Pd G3, tBuBrettphos Pd G3, tBuXPhos, Xantphos Pd G3, RuPhos Pd G3, Xantphos or BINAP, optionally in combination with Pd(OAc)₂, or Pd₂(dba)₃, in the presence of a suitable base such as Zn(OAc)₂, Cs₂CO₃, K₃PO₄ or t-BuONa in a suitable solvent such as dioxane, THE or toluene at between 70° C. and 120° C., optionally under microwave irradiation.

According to a third process, the compound of Formula (I) may be prepared from the compounds of Formulae (VI) and (VII) as shown in Scheme 3

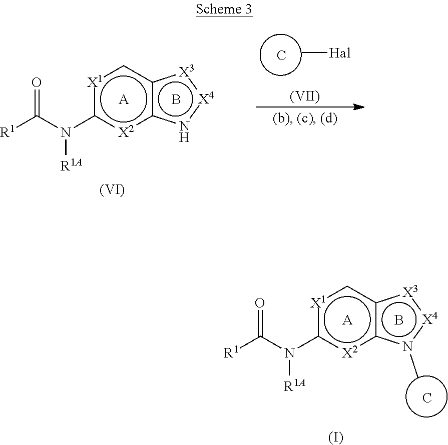

Hal is halogen, typically F, Cl, or Br.

The compound of Formula (I) may be prepared from the compound of Formula (VI) and the halide of Formula (VII), according to process step (b), a Buchwald-Hartwig reaction, as previously described in Scheme 2.

Alternatively, the compound of Formula (I) may be prepared from the compound of Formula (VI) and the halide of Formula (VII), by process step (c) an alkylation reaction, in the presence of a suitable inorganic or organic base and a suitable aprotic polar solvent at between rt and elevated temperature. Preferred conditions, comprise reaction of the compound of Formula (VI) with the compound of Formula (VII) in the presence of DBU or NaH, t-BuOK, Cs₂CO₃ or K₂CO₃ in DMSO, DMF or THF at between 5° and 100° C.

Alternatively, the compound of Formula (I) may be prepared from the compound of Formula (VI) and the halide of Formula (VII), by process step (d), an Ullmann-type, copper mediated coupling reaction. Typical conditions comprise, reaction of the compound of Formula (VI) with the compound of Formula (VII), a copper catalyst, optionally with a suitable ligand, optionally in the presence of a suitable inorganic or organic base in a suitable solvent at elevated temperature. Preferred conditions comprise, reaction of the compound of Formula (VI) with the compound of Formula (VII) in the presence of CuI, optionally in the presence of a suitable ligand such as N¹,N²-dimethylethane-1,2-diamine, trans-N,N′-dimethylcyclohexane-1,2-diamine, proline, 2,6-DFPAO or 2-(dimethylamino)acetic acid, optionally in the presence of a suitable inorganic base such as K₂CO₃ or K₃PO₄, in dioxane or DMSO at between 9° and 120° C., optionally under microwave irradiation.

According to a fourth process, when X³ is C—R³, compounds of Formula (I)(A) may be prepared from compounds of Formulae (VII), (VIII) and (IX) as shown in Scheme 4.

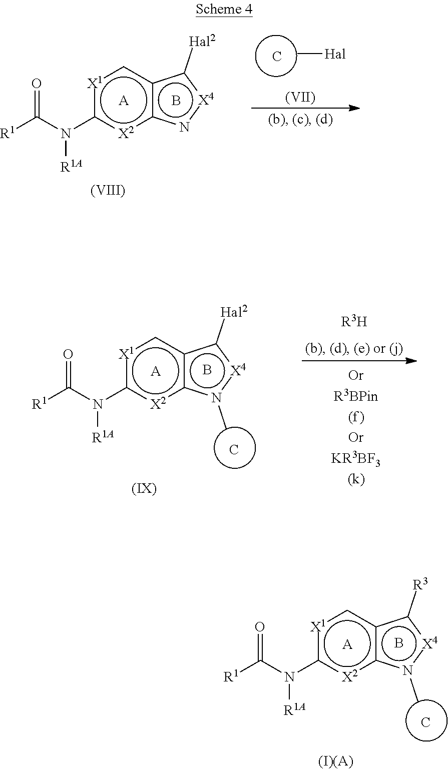

Hal² is halogen, preferably Cl, Br, or I.

The compound of Formula (IX) may be prepared from the compounds of Formulae (VII) and (VIII) according to process steps (b), (c) and (d) as previously described in Scheme 3.

When R³ is linked to ring B through a N atom, the compound of Formula (I) may be prepared by reaction of the compound of Formula (IX) with R³H according to process steps (b) or (d), as previously described in Scheme 2 and 3, or via process step (e) a photo-catalyzed nickel cross-coupling reaction or process step (j) a nickel catalyzed cross-coupling reaction. Preferred conditions comprise for step (e), reaction of the compound of Formula (IX) with R³H, in the presence of a suitable nickel catalyst such as (1,2-dimethoxyethane)nickel dibromide, Ru(bpy)₃(PF6)₂, or Ir[dF(CF₃)ppy]₂(dtbpy)PF₆, a suitable base such as DABCO or 2,6-lutidine under blue light at elevated temperatures, typically 80° C.

Preferred conditions comprise for step (j), reaction of the compound of Formula (IX) with R³H, in the presence of a suitable nickel catalyst such as (1,2-dimethoxyethane)nickel dibromide, Zn, a suitable organic base such as DABCO and DBU or MTBD, in a suitable aprotic solvent such as DMPU at about 55° C.

When R³ is linked to ring B through a C atom, the compound of Formula (I) may be prepared by reaction of the compound of Formula (IX) with R³Hal² according to process step (e) a photo-catalyzed nickel cross-coupling reaction as previously described.

When R³ is linked to ring B through a C atom, the compound of Formula (I) may be prepared from the compound of Formula (IX) and R³BPin according to process step (f) a palladium catalyzed, cross-coupling reaction, such as a Suzuki reaction. Typical cross-coupling reaction conditions comprise a palladium catalyst containing suitable phosphine ligands, in the presence of an inorganic base, in a suitable aqueous solvent at between rt and the reflux temperature of the reaction, optionally in the presence of microwave irradiation. Preferred conditions comprise, reaction of the compound of Formula (IX) and R³BPin, in the presence of Pd(dppf)Cl₂,Pd(dtbpf)Cl₂, CatacxiumA-Pd G2, bis(tricyclohexylphosphine)palladium(0) and a suitable base such as Cs₂CO₃, Na₂CO₃ or K₂CO₃ in a suitable solvent such as aqueous dioxane or toluene at between 70° C. and 100° C.

Alternatively, when R³ is linked to ring B through a C atom, the compound of Formula (I) may be prepared from the compound of Formula (IX) and KR³BF₃ according to process step (k) a palladium catalyzed, Suzuki-Miyaura cross-coupling reaction. Preferred conditions comprise, reaction of the compound of Formula (IX) and KR³BF₃, in the presence of Pd(dppf)Cl₂ and a suitable base such as Na₂CO₃ in a suitable solvent such as aqueous dioxane or toluene at between 70° C. and 100° C.

Alternatively, when R³ is linked to ring B through a C atom, the compound of Formula (I) may be prepared from the compound of Formula (IX) and R³ZnHal, according to process step (1), a palladium catalyzed, Negishi cross-coupling reaction. Preferred conditions comprise, reaction of the compound of Formula (IX) and R³ZnHal², in the presence of Pd(OAc)₂ and SPhos in a suitable solvent such as MeTHF at rt.

According to a fifth process compounds of Formula (II) may be prepared from compounds of Formulae (VII), (X), (XI), (XII) and (XIII) as shown in Scheme 5.

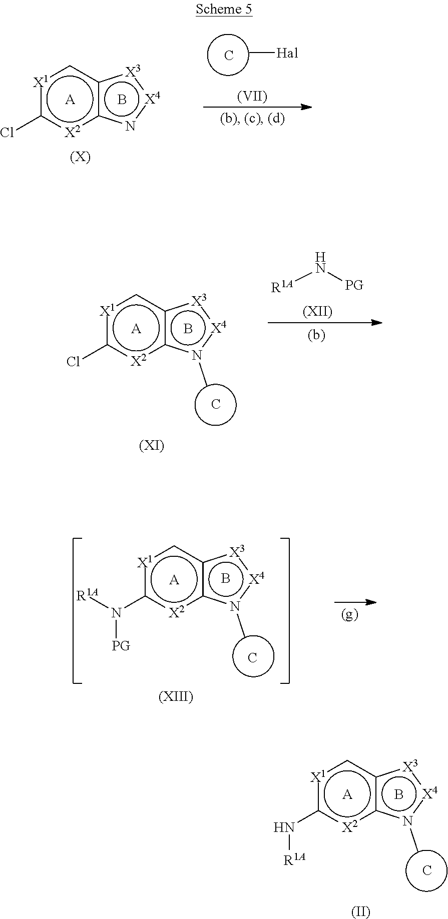

PG is a N protecting group, typically a carbamate and preferably Boc.

The compound of Formula (XI) may be prepared from the compound of Formula (X) and the compound of Formula (VII), according to process steps b) c) or d) as previously described in Schemes 2 and 3.

The compound of Formula (XIII) may be prepared from the compound of Formula (XI) and the protected amine of Formula (XII) according to process step (b) as previously described in Scheme 2.

The compound of Formula (II) may be prepared from the compound of Formula (XIII) by step (g) a de-protection reaction performed under standard conditions, such as treatment of the compound of Formula (XIII) with HCl in dioxane at rt.

In some circumstances, it may be possible to prepare the compound of Formula (II) directly from the compound of Formula (XI) as the protecting group, PG, is removed in-situ under conditions described for process step (b).

According to a sixth process, when X³ is C—R³, compounds of Formula (IV)(A) may be prepared from compounds of Formula (VII), (XIV), (XV), (XVI), and (XVII) as shown in Scheme 6.

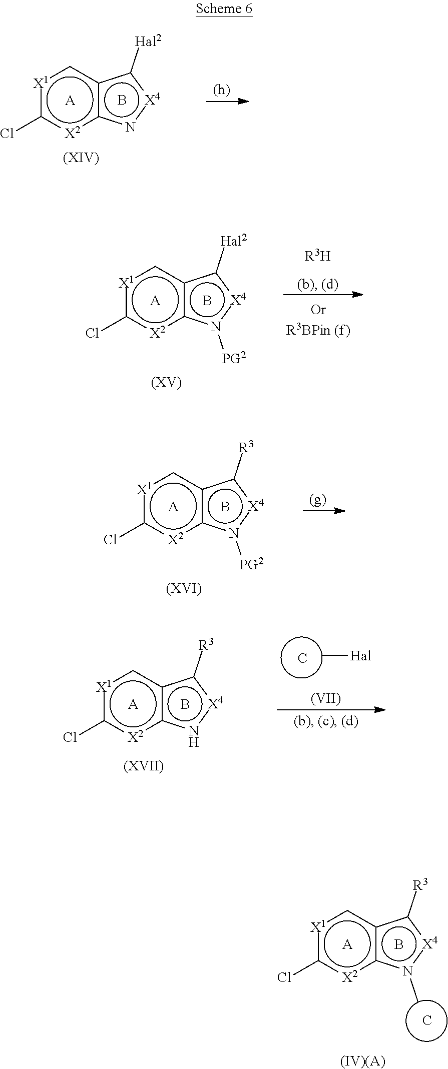

Hal² is halogen, preferably I or Br. PG² is a suitable aromatic amine protecting group, such as SEM, THP, tosyl or trityl, or Boc.

The compound of Formula (XV), may be obtained from the compound of Formula (XIV) according to process step (h), the protection of an aromatic amine group under standard conditions. For example, when PG² is SEM, the compound of Formula (XV) may be prepared by reaction of the compound of Formula (XIV) with SEMCl in the presence of NaH in THE at about rt. When PG² is THP, the compound of Formula (XV) may be prepared by reaction of the compound of Formula (XIV) with 3,4-dihydro-2H-pyran, in the presence of a catalyst such as MsOH, in a suitable solvent such as DCM, at elevated temperature such as 60° C. When PG² is tosyl, the compound of Formula (XV) may be prepared by reaction of the compound of Formula (XIV) with tosyl chloride in the presence of a strong base such as NaH in a suitable solvent such as DMF. When PG² is trityl, the compound of Formula (XV) may be prepared by reaction of the compound of Formula (XIV) with trityl chloride in the presence of a base such as Cs₂CO₃, in a suitable solvent such as DMF at about 100° C. When PG² is Boc, the compound of Formula (XV) may be prepared by reaction of the compound of Formula (XIV) with tert-butoxycarbonyl tert-butyl carbonate in the presence of a catalyst such as DMAP, in a suitable solvent such as DCM at rt

When R³ is C-linked to ring B through an N atom, the compound of Formula (XVI) may be obtained from the compound of Formula (XV) and R³Hal according to process step (b) or (d), as previously described in Scheme 2 and 3. Alternatively, when R³ is C-linked to ring B, the compound of Formula (XVI) may be obtained from the compound of Formula (XV) and R³BPin according to process step (f) as previously described in Scheme 4. Alternatively, When R³ is N-linked to ring B, the compound of Formula (XVI) may be obtained from the compound of Formula (XV) and R³H according to process step (d) as previously described in Scheme 3.

The compound of Formula (XVII) may be obtained from the compound of Formula (XVI) according to process step (g) a standard de-protection reaction. For example, when PG² is THP, Boc, or SEM, the de-protection may be achieved by reaction of the compound of Formula (XV) under acidic conditions, typically TFA or HCl in DCM, dioxane or HFIP at about rt. When PG² is trityl, the de-protection may be achieved by reaction of the compound of Formula (XV) with Et₃SiH and TFA in DCM at about rt. When PG² is tosyl, the de-protection may be achieved by reaction of compound of Formula (XV) with NaOH in MeOH at about 50° C.

The compound of Formula (IV)(A) may be obtained from the compounds of Formulae (III) and (XVII) according to process steps (b), (c) or (d) as previously described in Schemes 2 and 3.

According to a seventh process, wherein X³ is C—R³, compounds of Formula (VI)(A) may be prepared from the compounds of Formulae (VIII), (XVIII), (XIX) and (XX) as shown in Scheme 7.

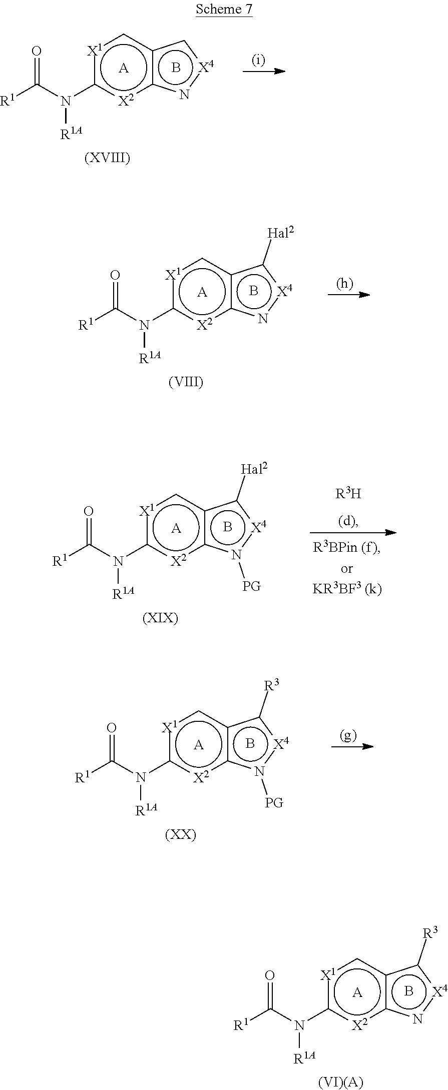

Hal² is halogen, preferably I or Br.

The compound of Formula (VIII) may be prepared from the compound of Formula (XVIII) by process step i) a halogenation reaction, such as a bromination or iodination. Typical conditions comprise reaction of the compound of Formula (XVIII) with N-bromo or N-iodosuccinimide in a suitable solvent, such as DMF at elevated temperature, such as 60° C. or with I₂ and a base such as KOH in DMF at rt.

The compound of Formula (XIX) may be prepared from the compound of Formula (VIII) according to process step (h), as previously described in Scheme 6.

When R³ is N-linked to ring B, the compound of Formula (XX) may be obtained from the compound of Formula (XIX) and R³H according to process step (d), as previously described in Scheme 6. Alternatively, when R³ is C-linked to ring B, the compound of Formula (XX) may be obtained from the compound of Formula (XIX) and R³BPin according to process step (f), or KR³BF₃ according to process step (k), as previously described in Scheme 4.

The compound of Formula (VI)(A) may be obtained from the compound of Formula (XX) by process step g) as previously described in Scheme 6.

According to an eighth process, wherein X³ is C—R³, compounds of Formula (VIII) may be prepared from the compounds of Formula (XXI) and (XXII) as shown in Scheme 8.

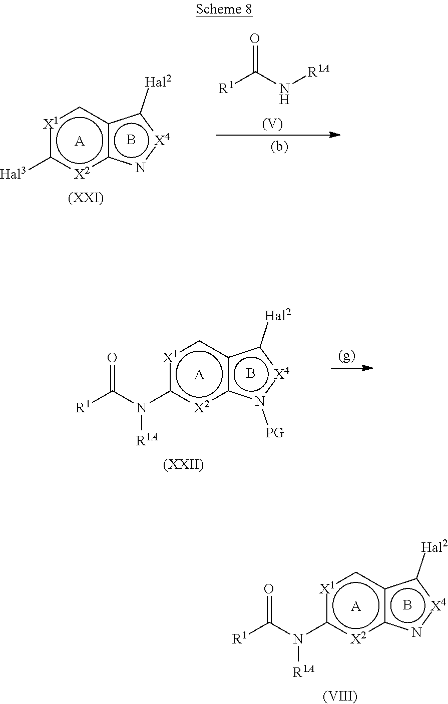

Hal³ is Halogen, preferably Br or Cl.

The compound of Formula (XXII) may be obtained from the compound of Formula (XXI) and the compound of Formula (V) according to process step b) as previously described in Scheme 2.

The compound of Formula (VIII) may be prepared from the compound of Formula (XXII) by process step (g) as previously described in Scheme 6.

According to a ninth process, when X³ is C—R³, compounds of Formula (IV)(A) may be prepared from compounds of Formula (VII), (XIV), (XV) and (XXIII) as shown in Scheme 9.

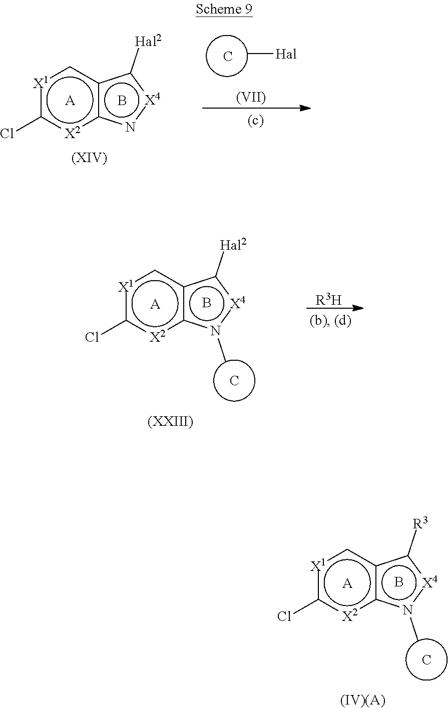

The compound of Formula (XXIII) may be prepared from the compounds of Formulae (XIV) and (VII), according to process step (c) as previously described in Scheme 3.

The compound of Formula (IV)(A) may be prepared from the compound of Formula (XXIII) and R³H according to process steps (b) or (d) as previously described in Schemes 2 and 3.

According to a tenth process, when X³ is C—R³, compounds of Formula (XX) may be prepared from compounds of Formula (XVI) and (V) as shown in Scheme 10.

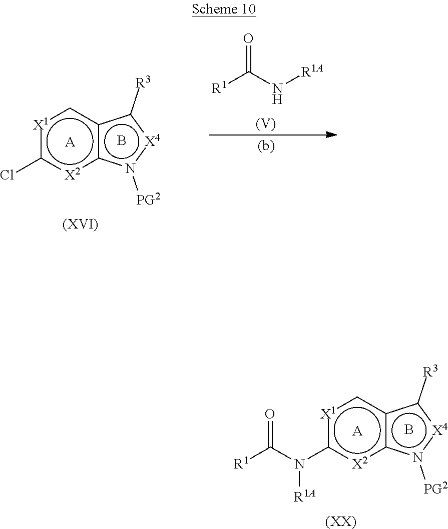

The compound of Formula (XX) may be prepared from the compounds of Formulae (XVI) and (V), according to process step (b) as previously described in Scheme 2.

According to an eleventh process, wherein X³ is N—R³, compounds of Formula (I)(B) may be prepared from the compounds of Formulae (V), (VI)(B), (VII), (XVI)(B) and (XX)(B) as shown in Scheme 11

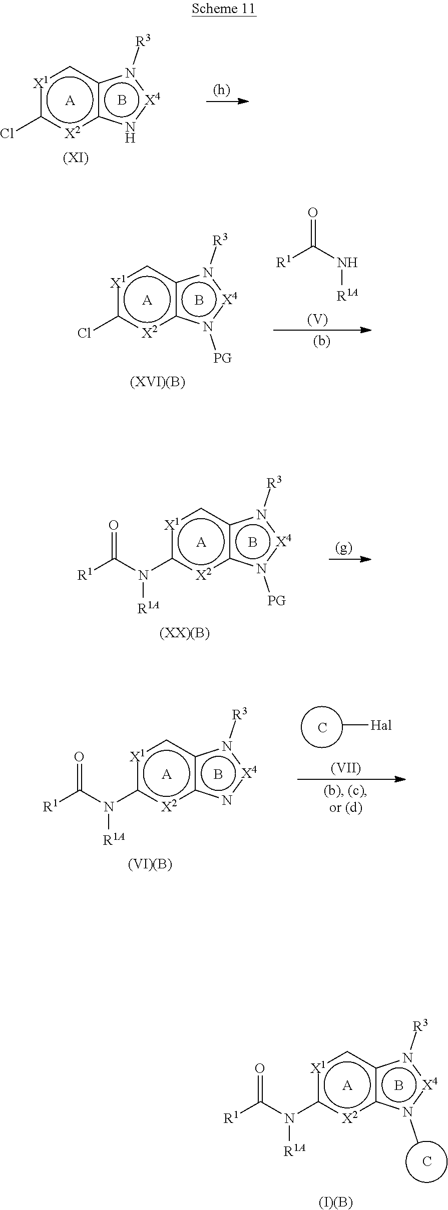

The compound of Formula (XVI)(B) may be prepared from the compound of Formula (XI) by process step (h), as previously described in Scheme 6.

The compound of Formula (XX)(B) may be prepared from the compounds of Formulae (XVI)(B) and (V), according to process step (b) as previously described in Scheme 2.

The compound of Formula (VI)(B) may be prepared from the compound of Formula (XX)(B) according to process step (g) as previously described in Scheme 6.

The compound of Formula (I)(B) may be prepared from the compounds of Formulae (VI)(B) and (VII) according to process step (b), (c) or (d) as previously described in Scheme 3.

The compounds of Formulae (III), (V), (VII), (X), (XI), (XII), (XIV), (XVIII), (XXI), R³Hal² and R³H, R³BPin, KR³BF₃, R³ZnHal², are either commercially available or may be prepared by analogy to methods known in the literature, or the methods described in the Experimental section below.

Compounds of Formula (I), (II) (IV), (VI), (IX), (X), (XI) (XVI), (XX) and (XXIII) may be converted to alternative compounds of Formula (I), (II) (IV), (VI), (IX), (X), (XI), (XVI), (XX) and (XXIII) by standard chemical transformations, known to those skilled in the art. Examples of these transformations include, but are not limited to:
Grignard reaction of an ester to provide a tertiary alcohol;
Reductive amination of a secondary amine with an aldehyde, to provide a tertiary amine;
Reductive amination of an aldehyde with a secondary amine to provide a tertiary amine;
Alkylation of a heteroatom, such as N or O, by reaction with an alkylating agent, such as an alkyl halide or alkyl triflate in the presence of an organic or inorganic base;
Reduction of an unsaturated bond by hydrogenation;
Reaction of an aryl or heteroaryl halide with a suitable boronate ester under typical Suzuki reaction conditions;
Reaction of an aryl or heteroaryl halide with an alkali metal alkoxide or aryl/heteroaryl alcohol in the presence of inorganic base, to provide an ether;
Fluorination of an alcohol, aldehyde or ketone with a suitable fluorinating agent, to provide an alkyl fluoride;
Reduction of an ester using a suitable reducing agent to provide an alcohol;
Sulfonation of an N atom to provide a sulfonamide; and
Acetylation of an N atom to provide an amide.

It will be appreciated by those skilled in the art that it may be necessary to utilize a suitable protecting group strategy for the preparation of compounds of Formula (I). Typical protecting groups may comprise, paramethoxybenzyl, benzyl or carbamate and preferably Boc or CBz for the protection of primary or secondary amines, a trityl, SEM or THP group for the protection of aromatic amines and a TBS or benzyl group for the protection of a primary alcohol.

It will be further appreciated that it may be necessary or desirable to carry out the transformations in a different order from that described in the schemes, or to modify one or more of the transformations, to provide the desired compound of the invention.

### HPLC Codes

| Code | Details |
|---|---|
| HPLC-A | Phenomenex Gemini NX C18 150 × 40 mm, 5 μm; MeCN/H₂O (0.05% |
| | NH₄OH + 10 mM NH₄HCO₃). Gradient (% MeCN) optimised for each |
| | compound. |
| HPLC-B | Boston Uni C18 150 × 40 mm, 5 μm; MeCN/H₂O (0.1% TFA). Gradient (% |
| | MeCN) optimised for each compound. |
| HPLC-C | Welch Xtimate C18 150 × 25 mm, 5 μm MeCN/H₂O (10 mM NH₄HCO₃). |
| | Gradient (% MeCN), over 10 min at 25 mL/min, optimised for each |
| | compound. |
| HPLC-D | Boston Green ODS 150 × 30 mm, 5 μm; MeCN/H₂O (0.1% TFA). Gradient |
| | (% MeCN) optimised for each compound. |
| HPLC-E | Phenomenex Synergi C18 150 × 30 mm, 4 μm; MeCN/H₂O (0.1% TFA). |
| | Gradient (% MeCN) optimised for each compound. |
| HPLC-F | Boston Prime C18 150 × 30 mm, 5 μm; MeCN/H₂O (0.05% NH4OH + 10 |
| | mM NH₄HCO₃. Gradient (% MeCN) optimised for each compound. |
| HPLC-G | Agela Durashell C18 150 × 25 mm, 5 μm; MeCN/H₂O (0.05% NH₄OH + 10 |
| | mM NH₄HCO₃). Gradient (% MeCN) optimised for each compound. |
| HPLC-H | YMC-Actus Triart C18 150 × 30 mm, 5 μm; MeCN/H₂O (TFA). Gradient (% |
| | MeCN) optimised for each compound. |
| HPLC-I | Boston Green ODS 150 × 30 mm, 5 μm; MeCN/H₂O (HCl). Gradient (% |
| | MeCN) optimised for each compound. |
| HPLC-J | Boston Prime C18 150 × 30 mm, 5 μm; MeCN/H₂O (0.05% HCl). Gradient |
| | (% MeCN) optimised for each compound. |
| HPLC-K | Phenomenex Synergi C18 150 × 30 mm, 4 μm; MeCN/H₂O (0.225% FA). |
| | Gradient (% MeCN) optimised for each compound. |
| HPLC-L | Boston Green ODS 150 × 30 mm, 5 μm; MeCN/H₂O (NH₄HCO₃). Gradient |
| | (% MeCN) optimised for each compound. |
| HPLC-M | Phenomenex Gemini NX C18 150 × 30 mm, 5 μm; MeCN/H₂O (0.05% HCl). |
| | Gradient (% MeCN) optimised for each compound. |
| HPLC-N | Phenomenex Gemini NX C18 75 × 30 mm, 3 μm; MeCN/H₂O (0.05% |
| | NH₄OHl). Gradient (% MeCN) optimised for each compound. |
| HPLC-O | Waters XBridge BEH C18 100 × 25 mm, 5 mm, MeCN/H₂O (HCO₂H). |
| | Gradient (% MeCN) optimised for each compound. |
| HPLC-P | Boston Prime C18 150 × 25 mm, 5 μm; MeCN/H₂O (NH₄HCO)₃. Gradient |
| | (% MeCN) optimised for each compound |
| HPLC-Q | Boston Green ODS 150 × 30 mm × 5 um; water (FA)-MeCN; Gradient (% |
| | MeCN) optimised for each compound. |
| HPLC-R | Waters XBridge BEH C18 100 × 30 mm × 10 um, MeCN/H₂O (NH₄HCO₃). |
| | Gradient (% MeCN) optimised for each compound. |
| HPLC-S | Waters XBridge BEH C18 150 × 25 mm × 10 um, MeCN/H₂O (NH₄HCO₃). |
| | Gradient (% MeCN) optimised for each compound. |
| HPLC-T | Column: Boston Prime C18 150*30 mm*5 um, Condition: water |
| | (NH₃•H₂O + NH₄HCO₃)-MeCN |
| HPLC-U | Column: Boston Prime C18 150*30 mm*5 um, Condition: water (FA)-MeCN |
| HPLC-V | Column: Waters Sunfire OBD C18 Prep 19 × 100 mm, 5 um. Condition: |
| | MeCN/H₂O (0.1% TFA) |
| HPLC-W | Column: Waters XSelect CSH C18 Prep 30 × 100 mm, 5 um OBD; MeCN/H₂O |
| | (0.2% NH₄OH) |
| HPLC-X | |

### General Synthetic Methods

### Preparation 1: N-(1-(tetrahydro-2H-pyran-2-yl)-1H-pyrazolo[4,3-c]pyridin-6-yl)acetamide

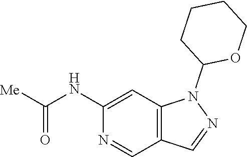 [SMILES: CC(=O)Nc1cc2c(cn1)cnn2C1CCCCO1]

A mixture of 6-chloro-1-tetrahydropyran-2-yl-pyrazolo[4,3-c]pyridine (1.00 g, 4.21 mmol), acetamide (497 mg, 8.42 mmol), Cs₂CO₃ (2.74 g, 8.42 mmol) and Brettphos Pd G3 (114.5 mg, 0.126 mmol) in dioxane (15 mL) was degassed with N₂, then heated at 100° C. in a sealed tube for 1 h. The cooled mixture was filtered, the filtrate was concentrated in vacuo and the crude was purified by chromatography on silica gel (0-70% EtOAc-EtOH 3:1 with 2% NH₄OH in heptane) to give N-(1-(tetrahydro-2H-pyran-2-yl)-1H-pyrazolo[4,3-c]pyridin-6-yl)acetamide (1.02 g, 93% yield) as a white powder. LCMS m/z=261.1 [M+H]⁺.

### Preparation 2: N-(1H-pyrazolo[4,3-c]pyridin-6-yl)acetamide

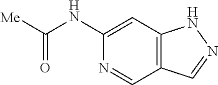 [SMILES: CC(=O)Nc1cc2[nH]ncc2cn1]

To a solution of N-(1-(tetrahydro-2H-pyran-2-yl)-1H-pyrazolo[4,3-c]pyridin-6-yl)acetamide (Preparation 1, 1.00 g, 3.84 mmol) in DCM (15 mL) was added TFA (8.76 g, 76.8 mmol) and the reaction was stirred at rt for 5 h. The mixture was concentrated in vacuo, the residue diluted with EtOAc and washed with aq. NaHCO₃. The organic layer was dried, filtered and concentrated in vacuo. The resulting solid was filtered off and washed with heptane/EtOAc (95/5) to give N-(1H-pyrazolo[4,3-c]pyridin-6-yl)acetamide (421 mg, 62% yield) as an off white solid. 1H NMR (400 MHz, MeOH-d₄) δ: 8.83 (s, 1H), 8.23 (s, 1H), 8.16 (s, 1H), 2.21 (s, 3H).

### Preparation 3: N-(3-iodo-1H-pyrazolo[4,3-c]pyridin-6-yl)acetamide

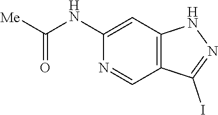 [SMILES: CC(=O)Nc1cc2[nH]nc(I)c2cn1]

A mixture of N-(1H-pyrazolo[4,3-c]pyridin-6-yl)acetamide (Preparation 2, 1.5 g, 8.51 mmol) and NIS (2.87 g, 12.8 mmol) in DMF (20 mL) was stirred at 60° C. for 6 h. The mixture was diluted with water and extracted with DCM (50 mL×3). The combined organics were washed with brine (100 mL), dried (Na₂SO₄) and evaporated to dryness in vacuo to afford N-(3-iodo-1H-pyrazolo[4,3-c]pyridin-6-yl)acetamide as a yellow solid (2 g, 77%) which was used without further purification. LCMS m/z=302.9 [M+H]⁺.

### Preparation 4: N-(3-iodo-1-trityl-1H-pyrazolo[4,3-c]pyridin-6-yl)acetamide

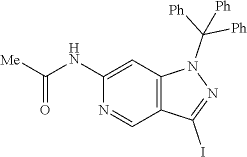 [SMILES: CC(=O)Nc1cc2c(cn1)c(I)nn2C(c1ccccc1)(c1ccccc1)c1ccccc1]

To a solution of N-(3-iodo-1H-pyrazolo[4,3-c]pyridin-6-yl)acetamide (Preparation 3, 5.0 g, 16.55 mmol) in DMF (50 mL) was added Cs₂CO₃ (10.8 g, 33.10 mmol) and trityl chloride (9.2 g, 33.1 mmol) and the mixture stirred at 100° C. for 16 h. The reaction was filtered, concentrated and poured into EtOAc (80 mL). The solid precipitated was collected by filtration and washed with H₂O to give N-(3-iodo-1-trityl-1H-pyrazolo[4,3-c]pyridin-6-yl)acetamide as a yellow solid (6.0 g, 66.6%). ¹H NMR (400 MHz, DMSO-d₆) δ: 10.48 (s, 1H), 8.48 (s, 1H), 7.34-7.29 (m, 11H), 7.28-7.15 (m, 5H), 1.94 (s, 3H).

### Preparation 5: N-(3-methyl-1-trityl-1H-pyrazolo[4,3-c]pyridin-6-yl)acetamide

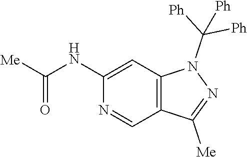 [SMILES: CC(=O)Nc1cc2c(cn1)c(C)nn2C(c1ccccc1)(c1ccccc1)c1ccccc1]

A mixture of N-(3-iodo-1-trityl-1H-pyrazolo[4,3-c]pyridin-6-yl)acetamide (Preparation 4, 250 mg, 0.459 mmol), 2,4,6-trimethyl-1,3,5,2,4,6-trioxatriborinane (576.5 mg, 4.59 mmol), K₂CO₃ (127 mg, 0.918 mmol) and Pd(dppf)Cl₂-DCM (37.5 mg, 0.046 mmol) in dioxane (5 mL) and H₂O (1 mL) was stirred at 120° C. for 12 h under N₂. The mixture was concentrated under reduced pressure and the residue purified by chromatography on silica gel (PE/EtOAc=1/1) to give N-(3-methyl-1-trityl-1H-pyrazolo[4,3-c]pyridin-6-yl)acetamide as a yellow solid (120 mg, 60%). ¹H NMR (400 MHz, CDCl₃) δ: 8.58-8.55 (m, 2H), 7.30-7.26 (m, 15H), 2.53 (s, 3H), 2.05 (s, 3H).

### Preparation 6: N-(3-methyl-1H-pyrazolo[4,3-c]pyridin-6-yl)acetamide

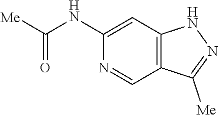 [SMILES: CC(=O)Nc1cc2[nH]nc(C)c2cn1]

To a solution of N-(3-methyl-1-trityl-1H-pyrazolo[4,3-c]pyridin-6-yl)acetamide (Preparation 5, 2.8 g, 6.47 mmol) in DCM (30 mL) was added TFA (8.9 g, 6.0 mL) and the resulting mixture was stirred at 30° C. for 6 h. The mixture was concentrated and purified by prep-HPLC-G (Gradient: 0 to 30%) to give N-(3-methyl-1H-pyrazolo[4,3-c]pyridin-6-yl)acetamide (1.1 g, 89.3% yield) as a white solid. ¹H NMR (500 MHz, CDCl₃) δ: 9.99 (br s, 1H), 8.68 (s, 1H), 8.28 (s, 1H), 8.20 (s, 1H), 2.61 (s, 3H), 2.25 (s, 3H).

### Preparation 7: 6-chloro-3-ethyl-1-(tetrahydro-2H-pyran-2-yl)-1H-pyrazolo[4,3-c]pyridine

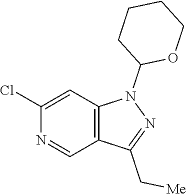 [SMILES: CCc1nn(C2CCCCO2)c2cc(Cl)ncc12]

To a solution of 6-chloro-3-ethyl-1H-pyrazolo[4,3-c]pyridine (500 mg, 2.75 mmol) in DCM (5 mL) was added 4-methylbenzenesulfonic acid;hydrate (52.4 mg, 0.275 mmol) and 3,4-dihydro-2H-pyran (694.71 mg, 8.26 mmol) and the mixture was stirred at 40° C. overnight. The reaction was diluted with DCM and water, the organic layer was separated, dried, filtered and concentrated in vacuo. The crude product was purified by chromatography on silica gel (0-70% EtOAc in heptane) to give 6-chloro-3-ethyl-1-(tetrahydro-2H-pyran-2-yl)-1H-pyrazolo[4,3-c]pyridine (686 mg, 93% yield) as a colorless oil. 1H NMR (400 MHz, CDCl₃) δ 8.79 (s, 1H), 7.48 (s, 1H), 5.59 (dd, J=2.64, 9.66 Hz, 1H), 4.12-4.02 (m, 1H), 3.74 (dt, J=2.89, 11.11 Hz, 1H), 3.02 (q, J=7.61 Hz, 2H), 2.54-2.39 (m, 1H), 2.19-2.02 (m, 2H), 1.83-1.71 (m, 3H), 1.42 (t, J=7.53 Hz, 3H). LCMS m/z=266.1 [M+H]⁺.

### Preparation 8: N-(3-ethyl-1-(tetrahydro-2H-pyran-2-yl)-1H-pyrazolo[4,3-c]pyridin-6-yl)acetamide

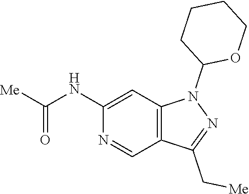 [SMILES: CCc1nn(C2CCCCO2)c2cc(NC(C)=O)ncc12]

N-(3-Ethyl-1-(tetrahydro-2H-pyran-2-yl)-1H-pyrazolo[4,3-c]pyridin-6-yl)acetamide was obtained as a white powder, 508 mg, 68% yield, from 6-chloro-3-ethyl-1-(tetrahydro-2H-pyran-2-yl)-1H-pyrazolo[4,3-c]pyridine (Preparation 7), following the procedure described in Preparation 1. LCMS m/z=289.1 [M+H]⁺.

### Preparation 9: N-(3-ethyl-1H-pyrazolo[4,3-c]pyridin-6-yl)acetamide

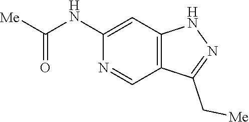 [SMILES: CCc1n[nH]c2cc(NC(C)=O)ncc12]

To a solution of N-(3-ethyl-1-(tetrahydro-2H-pyran-2-yl)-1H-pyrazolo[4,3-c]pyridin-6-yl)acetamide (Preparation 8, 500 mg, 1.73 mmol) in DCM (5 mL) was added TFA (2.98 g, 26.12 mmol) and the reaction stirred at rt overnight. The mixture was concentrated in vacuo, diluted with EtOAc, neutralized with 2M NaHCO₃, then washed with brine. The organic layer was dried and concentrated in vacuo. The crude was crystalized from MeCN to give N-(3-ethyl-1H-pyrazolo[4,3-c]pyridin-6-yl)acetamide (305 mg, 1.49 mmol, 86% yield) as a white solid. 1H NMR (400 MHz, MeOH-d₄) δ 8.80 (d, J=0.75 Hz, 1H), 8.14 (s, 1H), 3.02 (q, J=7.53 Hz, 2H), 2.20 (s, 3H), 1.40 (t, J=7.65 Hz, 3H).

### Preparation 10: N-(3-cyclopropyl-1-trityl-1H-pyrazolo[4,3-c]pyridin-6-yl)acetamide

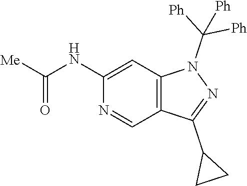 [SMILES: CC(=O)Nc1cc2c(cn1)c(C1CC1)nn2C(c1ccccc1)(c1ccccc1)c1ccccc1]

To a solution of N-(3-iodo-1-trityl-1H-pyrazolo[4,3-c]pyridin-6-yl)acetamide (Preparation 4, 4.0 g, 7.35 mmol) in dioxane (40 mL) and H₂O (8 mL) was added potassium cyclopropyltrifluoroborate (2.2 g, 14.7 mmol), Na₂CO₃ (1.6 g, 14.7 mmol) and Pd(dppf)Cl₂·DCM (600 mg, 0.735 mmol) and the resulting mixture stirred at 100° C. for 16 h under N₂. The mixture was poured into H₂O (80 mL) and extracted with EtOAc (3×100 mL×3). The combined organics were dried (Na₂SO₄) and evaporated to dryness in vacuo. The residue was purified by chromatography (SiO₂, 5-50% EtOAc/PE) to give N-(3-cyclopropyl-1-trityl-1H-pyrazolo[4,3-c]pyridin-6-yl)acetamide as a yellow solid (2.5 g, 74%). ¹H NMR (400 MHz, DMSO-d₆) δ: 10.31 (s, 1H), 8.75 (s, 1H), 7.31-7.26 (m, 11H), 7.24-7.15 (m, 5H), 2.29-2.25 (m, 1H), 1.92 (s, 3H), 1.00-0.97 (m, 2H), 0.90-0.88 (m, 2H).

### Preparation 11: N-(3-cyclopropyl-1H-pyrazolo[4,3-c]pyridin-6-yl)acetamide

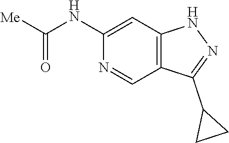 [SMILES: CC(=O)Nc1cc2[nH]nc(C3CC3)c2cn1]

TFA (11.9 g, 104 mmol) and triethylsilane (964 mg, 8.29 mmol) was added to a solution of give N-(3-cyclopropyl-1-trityl-1H-pyrazolo[4,3-c]pyridin-6-yl)acetamide (Preparation 10, 3.8 g, 8.29 mmol) in DCM (40 mL) and the resulting mixture stirred at 25° C. for 3 h. The reaction mixture was evaporated to dryness and the reside purified by chromatography (SiO₂, EtOAc) to give N-(3-cyclopropyl-1H-pyrazolo[4,3-c]pyridin-6-yl)acetamide as a yellow solid (1.6 g, 89%). ¹H NMR (500 MHz, DMSO-d₆) δ: 12.79 (s, 1H), 10.61 (s, 1H), 8.85 (s, 1H), 8.04 (s, 1H), 2.50-2.34 (m, 1H), 2.11 (s, 3H), 1.04-0.98 (m, 4H).

### Preparation 12: N-(3-iodo-1-(tetrahydro-2H-pyran-2-yl)-1H-pyrazolo[4,3-c]pyridin-6-yl)acetamide

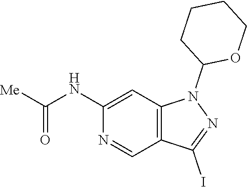 [SMILES: CC(=O)Nc1cc2c(cn1)c(I)nn2C1CCCCO1]

To a solution of N-(3-iodo-1H-pyrazolo[4,3-c]pyridin-6-yl)acetamide (Preparation 3, 2 g, 6.62 mmol) in THF (5 mL) and DCM (15 mL) was added MsOH (1.91 g, 19.86 mmol) and DHP (1.67 g, 19.86 mmol) and the mixture stirred at 60° C. for 5 h. The residue was poured into 10% of NaOH (50 mL) and the aqueous phase extracted with DCM (50 mL×5). The combined organics were washed with brine (2×20 mL), dried (Na₂SO₄) and concentrated in vacuum. The residue was purified by chromatography (SiO2, 33% EtOAc/PE) to give N-(3-iodo-1-(tetrahydro-2H-pyran-2-yl)-1H-pyrazolo[4,3-c]pyridin-6-yl)acetamide as a yellow solid (1.5 g, 58%). ¹H NMR (500 MHz, DMSO-d₆) δ: 10.73 (s, 1H), 8.54 (d, 1H), 8.29 (s, 1H), 5.77-5.75 (m, 1H), 3.88-3.85 (m, 1H), 3.74-3.67 (m, 1H), 2.37-2.26 (m, 1H), 2.13 (s, 3H), 2.02-1.93 (m, 2H), 1.81-1.71 (m, 1H), 1.61-1.51 (m, 2H).

### Preparation 13: 6-chloro-3-iodo-1H-pyrazolo[4,3-c]pyridine

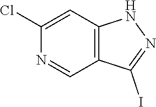 [SMILES: Clc1cc2[nH]nc(I)c2cn1]

To a solution of 6-chloro-1H-pyrazolo[4,3-c]pyridine (2.5 g, 16.28 mmol) in DMF (30 mL) was added NIS (3.7 g, 16.28 mmol) and the resulting mixture stirred at 70° C. for 3 h. The reaction was evaporated to dryness to give 6-chloro-3-iodo-1H-pyrazolo[4,3-c]pyridine as a brown solid (3.5 g, 77%) which was used directly to next step. LCMS m/z=279.9 [M+H]⁺.

### Preparation 14: 6-chloro-3-iodo-1-trityl-1H-pyrazolo[4,3-c]pyridine

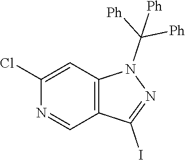 [SMILES: Clc1cc2c(cn1)c(I)nn2C(c1ccccc1)(c1ccccc1)c1ccccc1]

To a solution of 6-chloro-3-iodo-1H-pyrazolo[4,3-c]pyridine (Preparation 13, 3.5 g, 12.52 mmol) in DMF (40 mL) was added Cs₂CO₃ (8.2 g, 25.05 mmol) and TrtCl (6.9 g, 25.05 mmol) and the resulting mixture was stirred at 110° C. for 16 h. The reaction was filtered, concentrated and poured into EtOAc (80 mL) and the resulting solid collected by filtration to give 6-chloro-3-iodo-1-trityl-1H-pyrazolo[4,3-c]pyridine as a yellow solid (5.15 g, 79%). ¹H NMR (500 MHz, CDCl₃) δ: 8.51 (s, 1H), 7.23-7.29 (m, 15H), 5.99 (s, 1H).

### Preparation 15: 6-chloro-3-cyclopropyl-1-trityl-1H-pyrazolo[4,3-c]pyridine

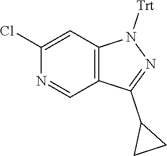

To a solution of 6-chloro-3-iodo-1-trityl-1H-pyrazolo[4,3-c]pyridine (Preparation 14, 4 g, 7.7 mmol) in dioxane (30 mL) and H₂O (6 mL) was added potassium cyclopropyltrifluoroborate (3.4 g, 23 mmol), Na₂CO₃ (1.63 g, 15.33 mmol) and Pd(dppf)Cl₂·DCM (626 mg, 0.767 mmol) and the reaction was stirred at 100° C. for 16 h under N₂. The cooled reaction was concentrated in vacuo, diluted with H₂O (50 mL) and extracted with EtOAc (50 mL×3). The combined organic phase was washed with brine (30 mL×2), dried over Na₂SO₄ and filtered. The mixture was concentrated and the residue was purified by chromatography on silica gel (PE/EtOAc=1/1) to give 6-chloro-3-cyclopropyl-1-trityl-1H-pyrazolo[4,3-c]pyridine (2.2 g, 65.8% yield) as a yellow solid. LCMS m/z=436.1 [M+H]⁺.

### Preparation 16: 6-chloro-3-cyclopropyl-1H-pyrazolo[4,3-c]pyridine

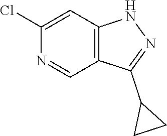 [SMILES: Clc1cc2[nH]nc(C3CC3)c2cn1]

To a solution of 6-chloro-3-cyclopropyl-1-trityl-1H-pyrazolo[4,3-c]pyridine (Preparation 15, 900 mg, 2.06 mmol) in DCM (10 mL) was added TFA (4.5 g, 39.49 mmol) and Et₃SiH (960.2 mg, 8.26 mmol) and the reaction was stirred at 25° C. for 5 h. The reaction was concentrated in vacuo and the residue was purified by silica column (PE/EtOAc=1/1) to give 6-chloro-3-cyclopropyl-1H-pyrazolo[4,3-c]pyridine (230 mg, 57.5% yield) as a yellow solid. ¹H NMR: (400 MHz, CDCl₃) δ ppm: 8.87 (s, 1H), 7.35 (s, 1H), 2.28-2.24 (m, 1H), 1.14-1.11 (m, 4H).

### Preparation 17: 4-(6-chloro-1H-pyrazolo[4,3-c]pyridin-3-yl)morpholine

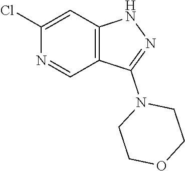 [SMILES: Clc1cc2[nH]nc(N3CCOCC3)c2cn1]

A mixture of 6-chloro-3-iodo-1-(tetrahydro-2H-pyran-2-yl)-1H-pyrazolo[4,3-c]pyridine (2 g, 5.50 mmol), morpholine (958.5 mg, 11.0 mmol), L-proline (126.7 mg, 1.10 mmol), K₂CO₃ (1.5 g, 11.0 mmol) and CuI (314.3 mg, 1.65 mmol) in DMSO (20 mL) was stirred at 100° C. for 2 h under N₂. The mixture was diluted with H₂O (30 mL) and extracted with EtOAc (50 mL×3). The combined organic phase was washed with brine (30 mL×2), dried over Na₂SO₄ and filtered. The filtrate was concentrated under reduced pressure and the residue was purified by chromatography on silica gel (PE/EtOAc=4/1) to give 4-(6-chloro-1-(tetrahydro-2H-pyran-2-yl)-1H-pyrazolo[4,3-c]pyridin-3-yl)morpholine (700 mg, 39.4% yield) as a yellow oil. ¹H NMR: (500 MHz, CDCl₃) δ: ppm 8.75 (s, 1H), 7.36 (s, 1H), 5.47-5.44 (m, 1H), 3.91-3.89 (m, 4H), 3.79-3.71 (m, 2H), 3.49-3.46 (m, 4H), 2.12-1.99 (m, 2H), 1.74-1.64 (m, 4H).

A mixture of 4-(6-chloro-1-(tetrahydro-2H-pyran-2-yl)-1H-pyrazolo[4,3-c]pyridin-3-yl)morpholine (700 mg, 2.17 mmol) in HCl/EtOAc (4 M, 10 mL) was stirred at 20° C. for 12 h. The mixture was concentrated under reduced pressure to give 4-(6-chloro-1H-pyrazolo[4,3-c]pyridin-3-yl)morpholine hydrochloride (520 mg) as a yellow solid. LCMS m/z=239.2 [M+H]⁺

### Preparation 18: 6-chloro-3-iodo-1-tosyl-1H-pyrrolo[3,2-c]pyridine

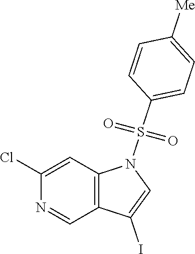 [SMILES: Cc1ccc(S(=O)(=O)n2cc(I)c3cnc(Cl)cc32)cc1]

To a solution of 6-chloro-3-iodo-1H-pyrrolo[3,2-c]pyridine (580 mg, 2.08 mmol) in DMF (5 mL) at 0° C. was added NaH (99.9 mg, 2.50 mmol, 60% purity) and stirred for 10 minutes. To this was added tosyl chloride (596 mg, 3.12 mmol) was added and the mixture stirred at 25° C. for 30 min. The mixture was treated with H₂O (20 mL) and extracted with EtOAc (25 mL×3). The combined organics were washed with brine (20 mL), dried (Na₂SO₄) and evaporated to dryness in vacuo. The residue was purified by column chromatography (SiO₂, 6-100% EtOAc/PE) to give 6-chloro-3-iodo-1-tosyl-1H-pyrrolo[3,2-c]pyridine as a yellow solid (680 mg, 75%). ¹H NMR (400 MHz, CDCl₃) δ: 8.43 (s, 1H), 7.89 (s, 1H), 7.81 (d, 2H), 7.66 (s, 1H), 7.33 (d, 2H), 2.41 (s, 3H).

### Preparation 19: 6-chloro-3-cyclopropyl-1-tosyl-1H-pyrrolo[3,2-c]pyridine

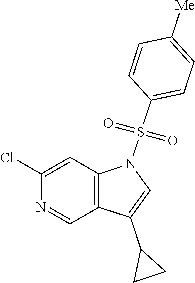 [SMILES: Cc1ccc(S(=O)(=O)n2cc(C3CC3)c3cnc(Cl)cc32)cc1]

To a solution of 6-chloro-3-iodo-1-tosyl-1H-pyrrolo[3,2-c]pyridine (Preparation 18, 730 mg, 1.69 mmol) in DMF (8 mL) was added cyclopropylzinc(II) bromide (0.5 M, 3.37 mL) and Pd(PPh₃)₄ (293 mg, 0.253 mmol) under N₂ and the mixture stirred at 70° C. for 2 h. The reaction was concentrated and diluted with H₂O (20 mL) and extracted with DCM (20 mL×2). The combined organics were washed with brine (20 mL×2) and evaporated to dryness in vacuo. The residue was purified by column chromatography (25% EtOAc/PE) to give 6-chloro-3-cyclopropyl-1-tosyl-1H-pyrrolo[3,2-c]pyridine as a white solid (280 mg, 48%). ¹H NMR (500 MHz, CDCl₃) δ: 8.66 (s, 1H), 7.88 (s, 1H), 7.76 (d, 2H), 7.31-7.28 (m, 2H), 7.18 (d, 1H), 2.39 (s, 3H), 1.87-1.84 (m, 1H), 0.99-0.95 (m, 2H), 0.69-0.66 (m, 2H).

### Preparation 20: N-(3-cyclopropyl-1-tosyl-1H-pyrrolo[3,2-c]pyridin-6-yl)acetamide

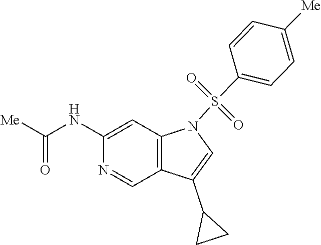 [SMILES: CC(=O)Nc1cc2c(cn1)c(C1CC1)cn2S(=O)(=O)c1ccc(C)cc1]

To a solution of 6-chloro-3-cyclopropyl-1-tosyl-1H-pyrrolo[3,2-c]pyridine (Preparation 19, 200 mg, 0.577 mmol) in dioxane (2 mL) was added acetamide (40.9 mg, 0.692 mmol), Cs₂CO₃ (376 mg, 1.15 mmol) and Brettphos Pd G3 (52.3 mg, 0.058 mmol) and the mixture stirred at 100° C. for 1 h under N₂. The mixture was concentrated under reduced pressure and purified by column chromatography (PE/EtOAc=10/1 to 1/1) to give N-(3-cyclopropyl-1-tosyl-1H-pyrrolo[3,2-c]pyridin-6-yl)acetamide (190 mg, 89%) as a white solid. ¹H NMR (400 MHz, CDCl₃) δ: 8.70 (s, 1H), 8.49 (s, 1H), 8.32 (s, 1H), 7.85 (d, 2H), 7.24 (d, 2H), 7.16 (s, 1H), 2.33 (s, 3H), 2.21 (s, 3H), 1.83-1.80 (m, 1H), 0.94-0.91 (m, 2H), 0.66-0.63 (m, 2H).

### Preparation 21: N-(3-cyclopropyl-1H-pyrrolo[3,2-c]pyridin-6-yl)acetamide

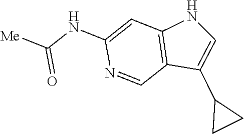 [SMILES: CC(=O)Nc1cc2[nH]cc(C3CC3)c2cn1]

To a solution of N-(3-cyclopropyl-1-tosyl-1H-pyrrolo[3,2-c]pyridin-6-yl)acetamide (Preparation 20, 180 mg, 0.487 mmol) in MeOH (2 mL) was added NaOH (39 mg, 0.974 mmol) and the mixture was stirred at 50° C. for 3 h. The mixture was concentrated under reduced pressure and the residue purified by column chromatography (PE/EtOAc=1/1 to 0/1) to give N-(3-cyclopropyl-1H-pyrrolo[3,2-c]pyridin-6-yl)acetamide (80 mg, 76%) as a white solid. ¹H NMR (400 MHz, CDCl₃) δ: 8.86 (s, 1H), 8.75 (s, 1H), 8.63 (s, 1H), 8.27 (s, 1H), 6.87 (s, 1H), 2.24 (s, 3H), 2.21-1.91 (m, 1H), 0.93-0.89 (m, 2H), 0.68-0.65 (m, 2H).

### Preparation 22: 6-chloro-1-(phenylsulfonyl)-1H-pyrrolo[3,2-c]pyridine

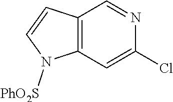

To a solution of 6-chloro-1H-pyrrolo[3,2-c]pyridine (250 g, 1.64 mol) in MeCN (500 mL) was added K₂CO₃ (566.1 g, 4.10 mol), followed by PhSO₂Cl (318.33 g, 1.802 mol) in dropwise and the reaction mixture was stirred at 25° C. for 4 h. The reaction mixture was poured into ice water (3.0 L). The precipitate was collected by filtration and dried to provide 6-chloro-1-(phenylsulfonyl)-1H-pyrrolo[3,2-c]pyridine (450 g, 93.8%) as yellow solid. LCMS m/z=293.1 [M+H]⁺

### Preparation 23: N-(1H-pyrrolo[3,2-c]pyridin-6-yl)acetamide

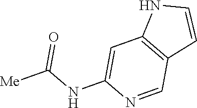 [SMILES: CC(=O)Nc1cc2[nH]ccc2cn1]

In to a reactor was placed dioxane (2000 mL), 6-chloro-1-(phenylsulfonyl)-1H-pyrrolo[3,2-c]pyridine (Preparation 22, 200 g, 683.2 mmol), AcNH₂ (161.42 g, 2.73 mol), K₂CO₃ (141.6 g, 1.025 mol), XantPhos (23.72 g, 41.0 mmol) and Pd(OAc)₂ (4.60 g, 20.5 mmol) in sequence. The mixture was heated to 110° C. and stirred for 16 h under N₂ atmosphere. The mixture was cooled to rt, a solution of NaOH (109.30 g, 2.73 mol) in H₂O (400 mL) was added and the resultant mixture was heated to 80° C. and stirred for 1 h. The cooled reaction mixture was diluted with EtOAc (3000 mL) and H₂O (400 mL). The insoluble substances were filtered off and the filtrate was separated. The aqueous phase was extracted with EtOAc (1000 mL×2), the combined organic extracts were dried over MgSO₄, filtered and concentrated. The precipitate was collected by filtration, the filter cake was dried to give N-(1H-pyrrolo[3,2-c]pyridin-6-yl)acetamide (90.0 g, 75.2%) as off-white solid. LCMS m/z=176.2 [M+H]⁺

### Preparation 24: N-(3-bromo-1H-pyrrolo[3,2-c]pyridin-6-yl)acetamide

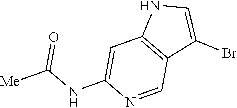 [SMILES: CC(=O)Nc1cc2[nH]cc(Br)c2cn1]

A stirred solution of N-(1H-pyrrolo[3,2-c]pyridin-6-yl)acetamide (80.0 g, 456.7 mmol) in DMF (500 mL) was added NBS (81.28 g, 456.7 mmol) in portions at 0° C. and once addition was complete, the reaction was stirred for 1 h. The mixture was poured into pre-cooled saturated aq. Na₂SO₃ (2 L), then filtered. The filter cake was suspended in saturated aq. Na₂CO₃ (2 L), stirred at rt for 1 h, then the mixture was filtered and dried to afford N-(3-bromo-1H-pyrrolo[3,2-c]pyridin-6-yl)acetamide (103.5 g, 89.2%) as off-white solid. LCMS m/z=254.1 [M+H]⁺

### Preparation 25: tert-butyl 6-chloro-3-iodo-1H-pyrrolo[3,2-c]pyridine-1-carboxylate

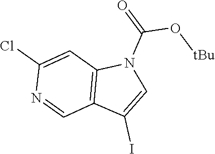 [SMILES: CC(C)(C)OC(=O)n1cc(I)c2cnc(Cl)cc21]

A solution of tert-butoxycarbonyl tert-butyl carbonate (7.84 g, 35.9 mmol) and DMAP (219 mg, 1.80 mmol) in DCM (50 mL) was slowly added to a stirring solution of 6-chloro-3-iodo-1H-pyrrolo[3,2-c]pyridine (5 g, 17.95 mmol) in DCM (100 mL) over 10 min and the reaction was then stirred at rt for 3 h. The reaction mixture was washed with 0.1N HCl, then brine. The organic layer was separated, dried and concentrated in vacuo. The crude was purified by chromatography on silica gel (0-60% EtOAc in heptane) to give tert-butyl 6-chloro-3-iodo-1H-pyrrolo[3,2-c]pyridine-1-carboxylate (6.51 g, 95% yield) as a white powder. LCMS m/z=379.0 [M+H]⁺ 1H NMR (400 MHZ, CDCl₃) δ 8.46 (s, 1H), 8.04 (s, 1H), 7.71 (s, 1H), 1.69 (s, 9H).

### Preparation 26: tert-butyl 6-chloro-3-cyclopropyl-1H-pyrrolo[3,2-c]pyridine-1-carboxylate

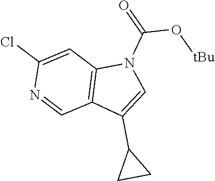 [SMILES: CC(C)(C)OC(=O)n1cc(C2CC2)c2cnc(Cl)cc21]

A mixture of tert-butyl 6-chloro-3-iodo-pyrrolo[3,2-c]pyridine-1-carboxylate (Preparation 25, 2.5 g, 6.60 mmol) and dichloro[bis(2-(diphenylphosphino)phenyl)ether]palladium(II) (47.3 mg, 0.066 mmol) in THF (50 mL) was purged with N₂, then bromo(cyclopropyl)zinc (0.5 M, 15.85 mL) was added slowly and the reaction mixture was stirred at rt overnight. The mixture was quenched with water, concentrated in vacuo, the residue was diluted with EtOAc, washed with water, the organic layer was then separated, dried and concentrated. The crude was purified by chromatography on silica gel (0-40% EtOAc in heptane) to give tert-butyl 6-chloro-3-cyclopropyl-1H-pyrrolo[3,2-c]pyridine-1-carboxylate (1.32 g, 68% yield) as a white solid. LCMS m/z=293.0 [M+H]⁺1H NMR (400 MHz, CDCl₃) δ 8.69 (d, 1H, J=0.8 Hz), 8.00 (br s, 1H), 7.21 (s, 1H), 2.00-1.80 (m, 1H), 1.67 (s, 9H), 1.00-0.90 (m, 2H), 0.7-0.7 (m, 2H).

### Preparation 27: 6-chloro-3-cyclopropyl-1H-pyrrolo[3,2-c]pyridine

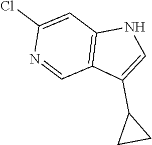 [SMILES: Clc1cc2[nH]cc(C3CC3)c2cn1]

To a solution of tert-butyl 6-chloro-3-cyclopropyl-1H-pyrrolo[3,2-c]pyridine-1-carboxylate (Preparation 26, 1.3 g, 4.44 mmol) in DCM (25 mL) was added TFA (3.72 g, 2.5 mL) and the reaction was stirred at rt overnight. The reaction mixture was concentrated under vacuum, the residue was diluted with EtOAc and washed with aq. NaHCO₃. The organic layer was separated, dried, and evaporated under reduced pressure to give 6-chloro-3-cyclopropyl-1H-pyrrolo[3,2-c]pyridine (910 mg, crude) which was used in the next step without purification. LCMS m/z=193.0 [M+H]⁺

### Preparation 28: 6-chloro-3-iodo-1-((2-(trimethylsilyl)ethoxy)methyl)-1H-pyrrolo[3,2-c]pyridine

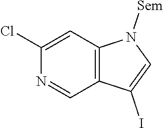

To a solution of 6-chloro-3-iodo-1H-pyrrolo[3,2-c]pyridine (2.2 g, 7.97 mmol) in THF (25 mL) was added NaH (478.3 mg, 11.96 mmol, 60% purity) at 25° C. and the solution stirred for 30 mins. SEMCl (1.6 g, 9.57 mmol) was added and the reaction stirred at 25° C. for 2 h. The mixture was quenched with H₂O (20 mL), extracted with EtOAc (20 mL×2), the combined organic phase was washed with brine (20 mL×2), dried over Na₂SO₄, filtered and concentrated under reduced pressure. The residue was purified by chromatography on silica gel (PE/EtOAc=3/1) to give 6-chloro-3-iodo-1-((2-(trimethylsilyl)ethoxy)methyl)-1H-pyrrolo[3,2-c]pyridine (3.2 g, 98.2% yield) as a yellow solid. ¹H NMR: (400 MHz, CDCl₃) δ ppm: 8.47 (s, 1H), 7.39 (s, 1H), 7.28 (s, 1H), 5.41 (s, 2H), 3.49-3.45 (m, 2H), 0.92-0.87 (m, 2H), −0.04 (s, 9H).

### Preparation 29: 2,6-dibromo-4-isopropoxypyridine

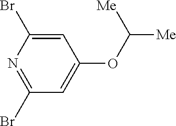 [SMILES: CC(C)Oc1cc(Br)nc(Br)c1]

To a solution of 2,4,6-tribromopyridine (247.4 mg, 4.12 mmol) in DMF (10 mL) was added NaH (329.3 mg, 8.23 mmol, 60% purity) at 0° C. and the mixture stirred for 30 mins. Isopropanol (1.3 g, 4.12 mmol) was added and the reaction stirred for 10 h at 25° C. The mixture was diluted with H₂O (30 mL), extracted with DCM (15 mL×3) and the combined organic extracts concentrated in vacuo. The crude was purified by silica gel chromatography to give 2,6-dibromo-4-isopropoxypyridine (400 mg, 32.9% yield) as colorless oil. ¹H NMR: (400 MHz, CDCl₃) δ: ppm 6.93 (s, 2H), 4.63-4.54 (m, 1H), 1.36 (d, J=6.4 Hz, 6H).

### Preparation 30: 2,6-dibromo-4-(2-methoxyethoxy)pyridine

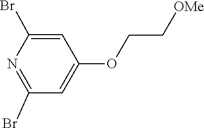 [SMILES: COCCOc1cc(Br)nc(Br)c1]

To a solution of 2,6-dibromopyridin-4-ol (1.0 g, 3.95 mmol) in DMF (15.0 mL) was added 1-bromo-2-methoxyethane (549.6 mg, 3.95 mmol) and Cs₂CO₃ (2.6 g, 7.91 mmol) and the reaction stirred at 90° C. for 12 h. The mixture was poured into H₂O (50 mL), extracted with EtOAc (3×50 mL), the combined organic layer was dried over anhydrous Na₂SO₄, filtered and concentrated in vacuo. The residue was purified by chromatography (PE/EtOAc=1/1) on silica gel to give 2,6-dibromo-4-(2-methoxyethoxy)pyridine (1.2 g, 98.4% yield) as colorless oil. ¹H NMR: (500 MHz, CDCl₃) δ ppm: 7.01 (s, 2H), 4.16-4.14 (m, 2H), 3.74-3.73 (m, 2H), 3.43 (s, 3H).

### Preparation 31: 3-(2-fluoropyridin-4-yl)tetrahydrofuran-3-ol

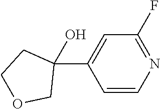 [SMILES: OC1(c2ccnc(F)c2)CCOC1]

To a solution of 4-bromo-2-fluoropyridine (1.0 g, 5.68 mmol) in THF (6.0 mL) was added n-BuLi (2.5 M, 2.5 mL) dropwise at −78° C. for 30 mins, then a solution of dihydrofuran-3(2H)-one (489 mg, 5.68 mmol) in THF (4.0 mL) was added slowly. The resulting mixture was allowed to warm to 10° C. and stirred for 1.5 h. The reaction was quenched with NH₄Cl (3 mL), concentrated under reduced pressure and the residue was purified by silica gel chromatography (PE/EtOAc=3/1 to 1/1) to give 3-(2-fluoropyridin-4-yl)tetrahydrofuran-3-ol (480 mg, 46.1% yield) as a colorless oil. LCMS m/z=184.3 [M+H]+;

### Preparation 32: 3-(4-chloropyridin-2-yl)tetrahydrofuran-3-ol

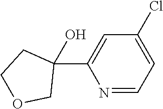 [SMILES: OC1(c2cc(Cl)ccn2)CCOC1]

3-(4-Chloropyridin-2-yl)tetrahydrofuran-3-ol was obtained as a brown oil, 560 mg, 54% yield, from 2-bromo-4-chloropyridine and dihydrofuran-3(2H)-one, following a similar procedure to that described in Preparation 31. 1H NMR: (500 MHz, CDCl₃) δ ppm: 8.43 (d, J=5.5 Hz, 1H), 7.52 (d, J=1.5 Hz, 1H), 7.26-7.25 (m, 1H), 4.81-4.76 (m, 1H), 4.20-4.17 (m, 2H), 4.16-4.01 (m, 1H), 3.93-3.91 (m, 1H), 2.42-2.36 (m, 1H), 2.29-2.25 (m, 1H).

### Preparation 33: 1-(6-bromopyridin-2-yl)-2,2-dimethylpropan-1-ol

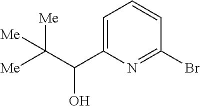 [SMILES: CC(C)(C)C(O)c1cccc(Br)n1]

1-(6-Bromopyridin-2-yl)-2,2-dimethylpropan-1-ol was obtained as a yellow oil, 754 mg, 73.2% yield, from 2,6-dibromopyridine and pivalaldehyde, following the procedure described in Preparation 31. LCMS m/z=244.2 [M+H]⁺

### Preparation 34: 3-(6-bromo-4-methoxypyridin-2-yl)tetrahydrofuran-3-ol

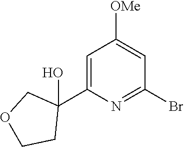 [SMILES: COc1cc(Br)nc(C2(O)CCOC2)c1]

To a solution of 2,6-dibromo-4-methoxypyridine (3.0 g, 11.24 mmol) in DCM (40 mL) was added n-BuLi (2.5 M, 4.95 mL) slowly at −70° C. for 30 mins under N₂. Then dihydrofuran-3(2H)-one (967.6 mg, 11.24 mmol) was added slowly at −70° C. for 1 hour under N₂. The mixture was quenched with NH₄Cl (sat, 30 mL), poured into H₂O (50 mL), extracted with EtOAc (3×80 mL), the combined organic layer was dried over anhydrous Na₂SO₄, filtered and concentrated, the residue was purified by chromatography (Petroleum ether/EtOAc=3/1) on silica gel to give 3-(6-bromo-4-methoxypyridin-2-yl)tetrahydrofuran-3-ol (2.6 g, 84.4% yield) as a yellow oil. ¹H NMR: (400 MHz, CDCl₃) δ ppm: 6.97-6.93 (m, 2H), 4.32 (s, 1H), 4.16-4.13 (m, 2H), 4.00-3.97 (m, 1H), 3.90-3.89 (m, 1H), 3.87 (s, 3H), 2.44-2.36 (m, 1H), 2.43-2.22 (m, 1H).

### Preparations 35 to 44

The compounds in the following table were prepared from the appropriate halopyridine and ketone, following a similar procedure to that described in Preparation 34.

| Prep | |
|---|---|
| No | Name, Structure, Starting Materials, Data |
| 35 | 1-(6-bromopyridin-2-yl)cyclopentan-1-ol |
| | 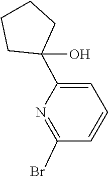 [SMILES: OC1(c2cccc(Br)n2)CCCC1] |
| | Halopyridine: 2,6-dibromopyridine. Ketone: cyclopentanone |
| | 1.2 g, 58.7% yield as a yellow oil. 1H NMR: (500 MHz, CDCl₃) |
| | δ ppm: 7.54 (t, J = 8.0 Hz, 1H), 7.38 (d, J = 7.0 Hz, 1H), |
| | 7.36 (d, J = 7.5 Hz, 1H), 3.86 (br s, 1H), 2.03-1.85 (m, 8H). |
| 36 | 3-(6-bromopyridin-2-yl)tetrahydrofuran-3-ol |
| | 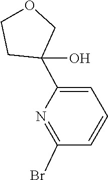 [SMILES: OC1(c2cccc(Br)n2)CCOC1] |
| | Halopyridine: 2,6-dibromopyridine. Ketone: dihydrofuran- |
| | 3(2H)-one yellow oil, 1.3 g, 63.1%. 1H NMR: (400 |
| | MHz, CDCl₃) δ ppm: 7.60 (t, J = 8.0 Hz, 1H), 7.48 (d, J = 7.6 Hz, |
| | 1H), 7.43 (d, J = 8.0 Hz, 1H), 4.35-4.34 (m, 1H), 4.19-4.16 |
| | (m, 2H), 4.00-3.94 (m, 1H), 3.91-3.90 (m, 1H), 2.46-2.39 (m, |
| | 1H), 2.29-2.27 (m, 1H). |
| 37 | 3-(6-bromo-4-isopropoxypyridin-2-yl)tetrahydrofuran-3-ol |
| | 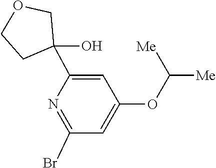 [SMILES: CC(C)Oc1cc(Br)nc(C2(O)CCOC2)c1] |
| | Halopyridine: 2,6-dibromo-4-isopropoxypyridine |
| | (Preparation 29) ketone: dihydrofuran-3(2H)-one |
| | 212 mg, 51.7% yield as yellow oil. 1H NMR: (400 MHz, |
| | CDCl₃) δ: ppm 6.91-6.88 (m, 2H), 4.66-4.60 (m, 1H), |
| | 4.40 (s, 1H), 4.19-4.09 (m, 2H), 3.98-3.95 (m, 1H), |
| | 3.90-3.87 (m, 1H), 2.41-2.34 (m, 1H), 2.25-2.22 (m, 1H), 1.36 |
| | (d, J = 6.0 Hz, 6H). |
| 38 | 3-(6-bromopyridin-2-yl)oxetan-3-ol |
| | 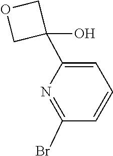 [SMILES: OC1(c2cccc(Br)n2)COC1] |
| | Halopyridine: 2,6-dibromopyridine. Ketone: oxetan-3-one |
| | white solid, 430 mg, 44.3% yield. 1H NMR: (400 MHz, |
| | CDCl₃) δ ppm: 7.93 (d, J = 7.6 Hz, 1H), 7.75-7.71 (m, |
| | 1H), 7.50 (d, J = 8.0 Hz, 1H), 5.26 (s, 1H), 5.08-5.06 |
| | (m, 2H), 4.72-4.69 (m, 2H). |
| 39 | 3-(6-bromo-4-methylpyridin-2-yl)oxetan-3-ol |
| | 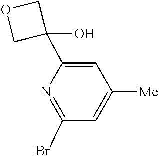 [SMILES: Cc1cc(Br)nc(C2(O)COC2)c1] |
| | Halopyridine: 2,6-dibromo-4-methylpyridine. Ketone: |
| | oxetan-3-one. white solid, 700 mg, 72.0% yield. |
| | 1H NMR: (500 MHz, CDCl₃) δ: ppm 7.73 (s, 1H), 7.33 |
| | (s, 1H), 5.31 (s, 1H), 5.06 (d, J = 7.5 Hz, 2H), 4.68 (d, J = 7.0 |
| | Hz, 2H), 2.44 (s, 3H). |
| 40 | 1-(6-bromopyridin-2-yl)cyclobutan-1-ol |
| | 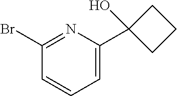 [SMILES: OC1(c2cccc(Br)n2)CCC1] |
| | Halopyridine: 2,6-dibromopyridine. Ketone: cyclobutane |
| | yellow oil, 1.4 g, 72.7% yield. 1H NMR: (500 MHz, |
| | CDCl₃) δ ppm: 7.57-7.62 (m, 1H), 7.51 (d, J = |
| | 6.5 Hz, 1H), 7.39 (d, J = 8.5 Hz, 1H), 4.31 (s, 1H), |
| | 2.52-2.49 (m, 4H), 2.07-2.05 (m, 1H), 1.88-1.82 (m, 1H). |
| 41 | 3-(6-bromo-4-methoxypyridin-2-yl)oxetan-3-ol |
| | 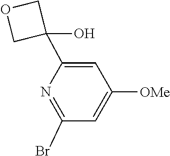 [SMILES: COc1cc(Br)nc(C2(O)COC2)c1] |
| | Halopyridine: 2,6-dibromo-4-methoxypyridine. Ketone: |
| | oxetan-3-one yellow solid, 620 mg, 63.3% yield |
| 42 | 3-(6-bromo-4-isopropoxypyridin-2-yl)oxetan-3-ol |
| | 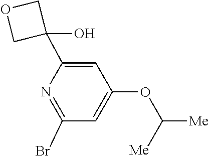 [SMILES: CC(C)Oc1cc(Br)nc(C2(O)COC2)c1] |
| | Halopyridine: 2,6-dibromo-4-isopropoxypyridine |
| | (Preparation 29). Ketone: oxetan-3-one |
| | yellow oil, 670 mg, 62.4% yield. 1H NMR: (500 MHz, |
| | CDCl₃) δ: ppm 7.36 (s, 1H), 6.96 (d, J = 2.5 Hz, 1H), |
| | 5.34 (s, 1H), 5.04-5.03 (m, 2H), 4.68-4.66 |
| | (m, 2H), 1.40 (d, J = 6.0 Hz, 6H). |
| 43 | 3-(6-bromo-4-(2-methoxyethoxy)pyridin-2-yl)oxetan-3-ol |
| | 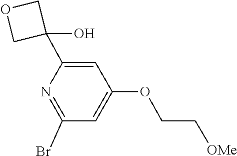 [SMILES: COCCOc1cc(Br)nc(C2(O)COC2)c1] |
| | Halopyridine: 2,6-dibromo-4-(2-methoxyethoxy)pyridine. |
| | Ketone: oxetan-3-one |
| | yellow solid, 461.5 mg, 39% yield. ¹H NMR: (400 MHz, |
| | CDCl₃) δ ppm: 7.46 (s, 1H), 7.04 (d, J = 2.0 Hz, |
| | 1H), 5.31 (s, 1H), 5.04-5.03 (m, 2H), 4.67-4.65 |
| | (m, 2H), 4.25-4.23 (m, 2H), 3.79-3.77 (m, 2H), 3.46 (s, 3H). |
| 44 | 3-(6-chloropyridin-2-yl)tetrahydrofuran-3-ol |
| | 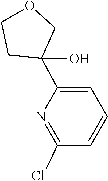 [SMILES: OC1(c2cccc(Cl)n2)CCOC1] |
| | Halopyridine: 2-bromo-6-chloropyridine. Ketone: |
| | dihydrofuran-3(2H)-one 6.90 g, 67% yield as a |
| | yellow oil. LCMS m/z = 200 [M + H]⁺ |

### Preparation 45: 3-(5-bromopyridin-3-yl)tetrahydrofuran-3-ol

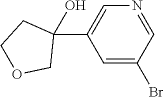 [SMILES: OC1(c2cncc(Br)c2)CCOC1]

To a solution of 3,5-dibromopyridine (1.0 g, 4.22 mmol) in THF (15.0 mL) was added iPrMgCl (2 M, 2.5 mL) dropwise at 0° C. and the solution was stirred at 0° C. for 2 h under N₂. Dihydrofuran-3(2H)-one (363.4 mg, 4.22 mmol) was added and the reaction was stirred at 25° C. for 20 h. The mixture was quenched with NH₄Cl (sat, 10 mL), poured into H₂O (20 mL) and extracted with EtOAc (3×20 mL). The combined organic layer was dried over anhydrous Na₂SO₄, filtered and concentrated and the residue was purified by column chromatography (PE/EtOAc=1/1) on silica gel to give 3-(5-bromopyridin-3-yl)tetrahydrofuran-3-ol (413.2 mg, 40.1% yield) as yellow oil. ¹H NMR: (400 MHz, CDCl₃) δ ppm: 8.62-8.60 (m, 2H), 8.04-8.02 (m, 1H), 4.24-4.14 (m, 2H), 3.98-3.96 (m, 1H), 3.88-3.86 (m, 1H), 2.43-2.32 (m, 2H).

### Preparation 46: 2-bromo-6-(prop-1-en-2-yl)pyridine

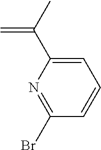

To a solution of 2-(6-bromopyridin-2-yl)propan-2-ol (0.4 g, 1.85 mmol) in DCM (6.0 mL) was added methanesulfonic anhydride (967.4 mg, 5.55 mmol) and TEA (1.03 mL, 7.40 mmol) and the reaction was stirred at 18° C. for 2 h. The pH of the reaction was adjusted 8 using sat. aq. NaHCO₃, the mixture diluted with H₂O (10 mL) and extracted with EtOAc (20 mL×3). The combined organic phase was washed with brine (10 mL), dried over Na₂SO₄, filtered and concentrated in vacuo. The residue was purified by column chromatography (PE/EtOAc=10/1 to 5/1) on silica gel to give 2-bromo-6-(prop-1-en-2-yl)pyridine (158 mg, 43.1% yield) as colorless gum. LCMS m/z=198.2 [M+H]⁺. 1H NMR (400 MHz, CDCl₃) δ ppm: 7.51-7.47 (m, 1H), 7.40-7.39 (m, 1H), 7.38-7.34 (m, 1H), 5.92 (s, 1H), 5.32-5.31 (m, 1H), 2.16 (s, 3H).

### Preparation 47: 2-(6-bromopyridin-2-yl)propane-1,2-diol

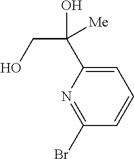 [SMILES: CC(O)(CO)c1cccc(Br)n1]

To a solution of 2-bromo-6-(prop-1-en-2-yl)pyridine (Preparation 46, 340 mg, 1.72 mmol) in acetone (2 mL) and water (4 mL) was added 4-methyl-4-oxido-morpholin-4-ium hydrate (243.6 mg, 1.80 mmol) and OsO₄ (2.2 mg, 8.58 μmol) and the mixture was stirred at 20° C. for 2 h. The reaction mixture was poured into water (20 mL), the mixture extracted with EtOAc (30 mL×3), the combined organic extracts were dried over Na₂SO₄, filtered and concentrated in vacuo. The crude was purified by column chromatography (PE/EtOAc=15/1 to 3/1) on silica gel to give 2-(6-bromopyridin-2-yl)propane-1,2-diol (386 mg, 97% yield) as colorless gum. ¹H NMR: (400 MHz, DMSO-d₆) δ ppm: 7.74-7.70 (m, 1H), 7.66-7.64 (m, 1H), 7.47-7.46 (m, 1H), 5.17 (s, 1H), 4.65 (t, J=5.6 Hz, 1H), 3.56-3.48 (m, 2H), 1.34 (s, 3H).

### Preparation 48: 2-fluoro-4-(3-methoxytetrahydrofuran-3-yl)pyridine

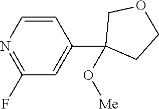 [SMILES: COC1(c2ccnc(F)c2)CCOC1]

To a solution of 3-(2-fluoropyridin-4-yl)tetrahydrofuran-3-ol (430 mg, 2.35 mmol) in THE (5.0 mL) was added NaH (103.4 mg, 2.59 mmol, 60% purity), then CH₃I (333.6 mg, 2.35 mmol) and the reaction mixture was stirred at 30° C. for 2 h. The reaction was quenched with aqueous NH₄Cl (3 mL) and concentrated under reduced pressure. The residue was purified by column chromatography on silica gel (PE/EtOAc=3/1) to give 2-fluoro-4-(3-methoxytetrahydrofuran-3-yl)pyridine (324 mg, 69.9% yield) as a colorless oil. LCMS m/z=198.3 [M+H]⁺

### Preparation 49: 2-bromo-6-(1-methoxy-2,2-dimethylpropyl)pyridine

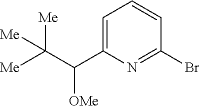 [SMILES: COC(c1cccc(Br)n1)C(C)(C)C]

2-Bromo-6-(1-methoxy-2,2-dimethylpropyl)pyridine was obtained as a colorless oil, 735 mg, 98.7% yield, from 1-(6-bromopyridin-2-yl)-2,2-dimethylpropan-1-ol, following a similar procedure to that described in Preparation 48.

### Preparation 50: 3-bromo-5-(3-methoxytetrahydrofuran-3-yl)pyridine

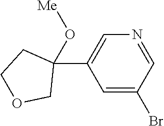 [SMILES: COC1(c2cncc(Br)c2)CCOC1]

3-Bromo-5-(3-methoxytetrahydrofuran-3-yl)pyridine was obtained as a yellow oil, 244.6 mg, 56.0% from 3-(5-bromopyridin-3-yl)tetrahydrofuran-3-ol, following a similar reaction to that described in Preparation 48. 1H NMR: (400 MHz, CDCl₃) δ ppm: 8.63 (d, J=2.0 Hz, 1H), 8.56 (d, J=1.6 Hz, 1H), 7.86 (t, J=2.5 Hz, 1H, 1H), 4.18-4.06 (m, 3H), 3.87-3.84 (m, 1H), 3.23 (s, 3H), 2.51-2.47 (m, 1H), 2.29-2.23 (m, 1H).

### Preparation 51: 2-bromo-6-(1-methoxycyclopentyl)pyridine

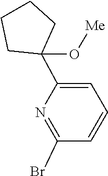 [SMILES: COC1(c2cccc(Br)n2)CCCC1]

2-Bromo-6-(1-methoxycyclopentyl)pyridine was obtained (1.1 g, 86.65% yield) as a colorless oil from 1-(6-bromopyridin-2-yl)cyclopentan-1-ol (Preparation 35) and methyl iodide, following the procedure described in Preparation 48. 1H NMR: (400 MHz, CDCl₃) δ ppm: 7.55-7.47 (m, 2H), 7.33 (d, J=8.0 Hz, 1H), 3.09 (s, 3H), 2.11-2.03 (m, 4H), 1.81-1.78 (m, 4H).

### Preparation 52: 2-bromo-6-(1-methoxycyclobutyl)pyridine

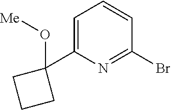 [SMILES: COC1(c2cccc(Br)n2)CCC1]

2-Bromo-6-(1-methoxycyclobutyl)pyridine was obtained as a colorless oil from 1-(6-bromopyridin-2-yl)cyclobutan-1-ol (Preparation 40) and MeI, following the procedure described in Preparation 48. 1H NMR: (400 MHz, CDCl₃) δ ppm: 7.54 (t, J=8.0 Hz, 1H), 7.42 (d, J=7.6 Hz, 1H), 7.37 (d, J=8.4 Hz, 1H), 3.04 (s, 3H), 2.56-2.52 (m, 2H), 2.34-2.31 (m, 2H), 1.89-1.82 (m, 2H).

### Preparation 53: 2-bromo-6-(1,2-dimethoxypropan-2-yl)pyridine

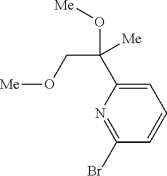 [SMILES: COCC(C)(OC)c1cccc(Br)n1]

2-Bromo-6-(1,2-dimethoxypropan-2-yl)pyridine was obtained (326 mg, 80.8% yield) as a colorless gum, from 2-(6-bromopyridin-2-yl)propane-1,2-diol (Preparation 47), following a similar procedure to that described in Preparation 48. 1H NMR (400 MHz, CDCl₃) δ ppm: 7.56-7.51 (m, 2H), 7.36-7.35 (m, 1H), 3.64 (s, 2H), 3.33 (s, 3H), 3.25 (s, 3H), 1.54 (s, 3H).

### Preparation 54: 4-chloro-2-(3-methoxytetrahydrofuran-3-yl)pyridine

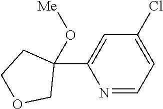 [SMILES: COC1(c2cc(Cl)ccn2)CCOC1]

NaH (144.3 mg, 3.61 mmol, 60% purity) was added to a solution of 3-(4-chloropyridin-2-yl)tetrahydrofuran-3-ol (Preparation 32, 360 mg, 1.8 mmol) in THF (10 mL) at 0° C. and the solution stirred at 20° C. for 10 mins. CH₃I (383.9 mg, 2.7 mmol) was added and the reaction was stirred at 20° C. for 2 h. The mixture was quenched with water (10 mL), extracted with EtOAc (10 mL×3) and the organic layers was washed by brine (20 mL), filtered and concentrated in vacuo. The residue was purified by column chromatography (PE/EtOAc=15/1 to 3/1) on silica gel to give 4-chloro-2-(3-methoxytetrahydrofuran-3-yl)pyridine (300 mg, 77.900 yield) as brown oil. 1H NMR: (400 MHz, CDCl₃) δ ppm: 8.47 (d, J=5.6 Hz, 1H), 7.55 (d, J=1.2 Hz, 1H), 7.23-7.22 (m, 1H), 4.17-3.99 (m, 4H), 3.21 (s, 3H), 2.64-2.56 (m, 1H), 2.39-2.33 (m, 1H).

### Preparations 55 to 61

The compounds in the following table were prepared from the appropriate alcohol and methyl iodide, following a similar procedure to that described in Preparation 54.

| Preparation | Name, Structure, Starting Materials, Data |
|---|---|
| 55 | 2-bromo-6-(3-methoxytetrahydrofuran-3-yl)pyridine |
| | 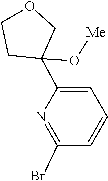 [SMILES: COC1(c2cccc(Br)n2)CCOC1] |
| | Alcohol: 3-(6-bromopyridin-2-yl)tetrahydrofuran-3-ol (Preparation 36) |
| | yellow oil, 3 g, 94.6% yield. 1H NMR: (400 MHz, MeOH-d₄) δ ppm: 7.73- |
| | 7.69 (m, 1H), 7.57-7.49 (m, 2H), 4.12-4.07 (m, 3H), 3.95-3.92 (m, 1H), 3.18 |
| | (s, 3H), 2.58-2.52 (m, 1H), 2.42-2.36 (m, 1H). |
| 56 | 2-bromo-6-(3-methoxyoxetan-3-yl)pyridine |
| | 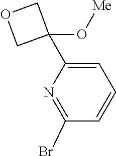 [SMILES: COC1(c2cccc(Br)n2)COC1] |
| | Alcohol: 3-(6-bromopyridin-2-yl)oxetan-3-ol (Preparation 38) |
| | 416.3 mg, 91.2% yield as colorless oil. 1H NMR: (400 MHz, CDCl₃) δ ppm: |
| | 7.61-7.58 (m, 1H), 7.46-7.41 (m, 1H), 7.40-7.39 (m, 1H), 5.05-5.04 (m, 2H), |
| | 4.86-4.84 (m, 2H), 3.24 (s, 3H). |
| 57 | 2-bromo-6-(1-methoxycyclopropyl)pyridine |
| | 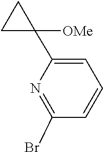 [SMILES: COC1(c2cccc(Br)n2)CC1] |
| | Alcohol: 1-(6-bromopyridin-2-yl)cyclopropan-1-ol |
| | 80 mg, 75.1% yield as brown oil. 1H NMR: (500 MHz, CDCl₃) δ ppm: |
| | 7.98-7.97 (m, 1H), 7.65-7.64 (m, 1H), 7.56-7.49 (m, 1H), 3.35 (s, 3H), 1.39- |
| | 1.36 (m, 2H), 1.26-1.23 (m, 2H). |
| 58 | 2-(6-bromopyridin-2-yl)-1-methoxypropan-2-ol |
| | 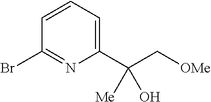 [SMILES: COCC(C)(O)c1cccc(Br)n1] |
| | Alcohol: 2-(6-bromopyridin-2-yl)propane-1,2-diol (Preparation 47) |
| | 135.2 mg, 31.9% yield as colorless oil. 1H NMR: (400 MHz, CDCl₃) δ ppm: |
| | 7.67-7.51 (m, 2H), 7.36 (d, J = 6.8 Hz, 1H), 3.90 (s, 1H), 3.75-3.73 (m, 1H), |
| | 3.49-3.47 (m, 1H), 3.33 (s, 3H), 1.49 (s, 3H). |
| 59 | 2-bromo-4-isopropoxy-6-(3-methoxyoxetan-3-yl)pyridine |
| | 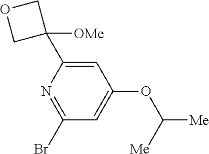 [SMILES: COC1(c2cc(OC(C)C)cc(Br)n2)COC1] |
| | Alcohol: 3-(6-bromo-4-isopropoxypyridin-2-yl)oxetan-3-ol (Preparation 42) |
| | 450 mg, 71.5% yield as a colorless oil. 1H NMR: (500MHz, CDCl₃) δ: ppm |
| | 6.92 (d, J = 1.5 Hz, 1H), 6.86 (d, J = 2.0 Hz 1H), 5.02 (d, J = 7.0 Hz, 2H), |
| | 4.80 (d, J = 7.0 Hz, 2H), 4.66-4.61 (m, 1H), 3.24 (s, 3H), 1.36 (d, J = 6.0 Hz, |
| | 6H). |
| 60 | 2-bromo-4-(2-methoxyethoxy)-6-(3-methoxyoxetan-3-yl)pyridine |
| | 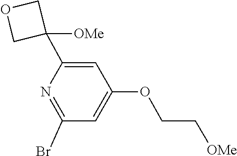 [SMILES: COCCOc1cc(Br)nc(C2(OC)COC2)c1] |
| | Alcohol: 3-(6-bromo-4-(2-methoxyethoxy)pyridin-2-yl)oxetan-3-ol |
| | (Preparation 43) |
| | 461.2 mg, 79.4% yield as colorless oil. 1H NMR: (400 MHz, CDCl₃) δ ppm: |
| | 6.99-6.95 (m, 2H), 5.03-5.01 (m, 2H), 4.82-4.80 (m, 2H), 4.18-4.16 (m, 2H), |
| | 3.76-3.74 (m, 2H), 3.44 (s, 3H), 3.23 (s, 3H). |
| 61 | 2-chloro-6-(3-methoxytetrahydrofuran-3-yl)pyridine |
| | 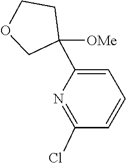 [SMILES: COC1(c2cccc(Cl)n2)CCOC1] |
| | Alcohol: 3-(6-chloropyridin-2-yl)tetrahydrofuran-3-ol (Preparation 44) |
| | 6.30 g, 85% yield as yellow oil. LCMS m/z = 214 [M + H]⁺ |

### Preparation 62: 2-bromo-6-(3-(difluoromethoxy)tetrahydrofuran-3-yl)pyridine

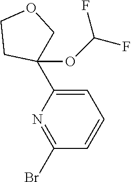 [SMILES: FC(F)OC1(c2cccc(Br)n2)CCOC1]

To a solution of 3-(6-bromopyridin-2-yl)tetrahydrofuran-3-ol (Preparation 36, 400 mg, 1.64 mmol) in THF (10 mL) was added NaH (98.3 mg, 2.46 mmol, 60% purity) at −60° C., then CHF₂Cl (708.5 mg, 8.19 mmol) was added. The resulting mixture was stirred at 25° C. for 12 h then concentrated under reduced pressure. The residue was purified by chromatography on silica gel (PE/EtOAc=10/1 to 5/1) to give 2-bromo-6-(3-(difluoromethoxy)tetrahydrofuran-3-yl)pyridine (75 mg, 15.6% yield) as a white solid. 1H NMR (500 MHz, CDCl₃) δ ppm: 7.63-7.56 (m, 2H), 7.45 (d, J=7.5 Hz, 1H), 6.37 (t, J=73.5 Hz, 1H), 4.26-4.20 (m, 2H), 4.16-4.14 (m, 1H), 4.07-4.05 (m, 1H), 2.90-2.83 (m, 1H), 2.51-2.47 (m, 1H).

### Preparation 63: 2-bromo-6-(3-fluorotetrahydrofuran-3-yl)pyridine

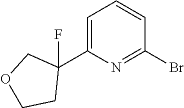 [SMILES: FC1(c2cccc(Br)n2)CCOC1]

To a solution of 3-(6-bromopyridin-2-yl)tetrahydrofuran-3-ol (Preparation 36, 2 g, 8.19 mmol) in DCM (20 mL) was added DAST (1.3 g, 8.19 mmol) at 0° C. and the reaction stirred at 25° C. for 12 h. The mixture was quenched with Na₂CO₃ (20 mL), extracted with DCM (2×30 mL), the combined organic phase was washed with brine (2×15 mL), dried over Na₂SO₄, filtered and concentrated under reduced pressure. The residue was purified by chromatography on silica gel (PE/EtOAc=5/1) to give 2-bromo-6-(3-fluorotetrahydrofuran-3-yl)pyridine (1.4 g, 67% yield) as a yellow oil. 1H NMR: (400 MHz, CDCl₃) δ: ppm 7.61-7.56 (m, 2H), 7.44-7.39 (m, 1H), 4.21-4.18 (m, 3H), 4.12-4.11 (m, 1H), 2.78-2.66 (m, 1H), 2.44-2.38 (m, 1H).

### Preparation 64: 2-bromo-6-(3-fluorotetrahydrofuran-3-yl)-4-methylpyridine

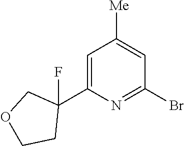 [SMILES: Cc1cc(Br)nc(C2(F)CCOC2)c1]

2-Bromo-6-(3-fluorotetrahydrofuran-3-yl)-4-methylpyridine was obtained, 1.4 g, 66.2% yield, as a yellow oil, from 3-(6-bromo-4-methylpyridin-2-yl)tetrahydrofuran-3-ol (Preparation 123) following the procedure described in Preparation 63. 1H NMR: (400 MHz, CDCl₃) δ: ppm 7.40 (s, 1H), 7.26-7.24 (m, 1H), 4.20-4.17 (m, 3H), 4.10-4.08 (m, 1H), 2.78-2.63 (m, 1H), 2.36-2.30 (m, 4H).

### Preparation 65: 2-bromo-6-(3-fluorotetrahydrofuran-3-yl)-4-methoxypyridine

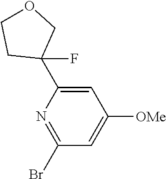 [SMILES: COc1cc(Br)nc(C2(F)CCOC2)c1]

2-Bromo-6-(3-fluorotetrahydrofuran-3-yl)-4-methoxypyridine was obtained, as a yellow solid, 1.7 g, 63.8% yield, from 3-(6-bromo-4-methoxypyridin-2-yl)tetrahydrofuran-3-ol (Preparation 34), following a similar procedure to that described in Preparation 63. ¹H NMR: (400 MHz, CDCl₃) δ ppm: 7.11 (d, J=2.0 Hz, 1H), 6.91 (d, J=2.0 Hz, 1H), 4.20-4.16 (m, 3H), 4.13-4.09 (m, 1H), 3.87 (s, 3H), 2.78-2.65 (m, 1H), 2.40-2.31 (m, 1H).

### Preparation 66: 2-bromo-6-(3-fluorotetrahydrofuran-3-yl)-4-isopropoxypyridine

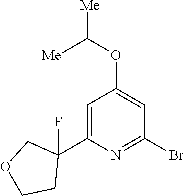 [SMILES: CC(C)Oc1cc(Br)nc(C2(F)CCOC2)c1]

To a solution of 3-(6-bromo-4-isopropoxypyridin-2-yl)tetrahydrofuran-3-ol (Preparation 37, 210 mg, 0.695 mmol) in DCM (10 mL) was added DAST (224.1 mg, 1.39 mmol) and the reaction was stirred at 25° C. for 10 h. The mixture was added to NaHCO₃ aqueous solution (20 mL) and extracted with DCM (10 mL×3). The combined organic phase was concentrated in vacuo and the crude was purified by silica gel chromatography (PE/EtOAc=20/1) to give 2-bromo-6-(3-fluorotetrahydrofuran-3-yl)-4-isopropoxypyridine (185 mg, 87.5% yield) as yellow oil. ¹H NMR: (500 MHz, CDCl₃) δ: ppm 7.07-7.06 (m, 1H), 6.87 (d, J=2.0 Hz, 1H), 4.19-4.16 (m, 2H), 4.15-4.11 (m, 2H), 4.10-4.08 (m, 1H), 2.76-2.67 (m, 1H), 2.38-2.29 (m, 2H), 1.37 (d, J=6.0 Hz, 6H).

### Preparation 67: 2-bromo-6-(3-fluorooxetan-3-yl)-4-methylpyridine

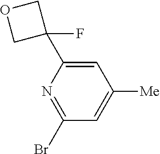 [SMILES: Cc1cc(Br)nc(C2(F)COC2)c1]

To a solution of 3-(6-bromo-4-methylpyridin-2-yl)oxetan-3-ol (Preparation 39, 680 mg, 2.79 mmol) in DCM (10 mL) was added DAST (898.1 mg, 5.57 mmol) at 0° C. and the reaction was stirred at 20° C. for 12 h. The mixture was quenched with Na₂CO₃ (10 mL) and extracted with DCM (2×10 mL). The combined organic phase was washed with brine (2×10 mL), dried over Na₂SO₄, filtered and the filtrate was concentrated under reduced pressure. The residue was purified by chromatography on silica gel (PE/EtOAc=5/1) to give 2-bromo-6-(3-fluorooxetan-3-yl)-4-methylpyridine (280 mg, 40.8% yield) as a yellow oil. ¹H NMR: (500 MHz, CDCl₃) δ: ppm 7.31 (s, 1H), 7.27 (s, 1H), 5.16 (d, J=8.0 Hz, 1H), 5.11 (d, J=9.0 Hz, 1H), 4.97 (d, J=8.5 Hz, 1H), 4.92 (d, J=8.5 Hz, 1H), 2.36 (s, 3H).

### Preparation 68 to 72

The compounds in the following table were prepared from the appropriate alcohol and DAST, following a similar procedure to that described in Preparation 67.

| Preparation | |
|---|---|
| No | Name, Structure, Starting Materials, Data |
| 68 | 2-bromo-6-(3-fluorooxetan-3-yl)-4-methoxypyridine |
| | 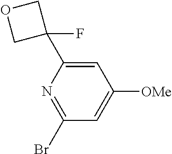 [SMILES: COc1cc(Br)nc(C2(F)COC2)c1] |
| | Alcohol: 3-(6-bromo-4-methoxypyridin-2-yl)oxetan-3-ol (Preparation 41) |
| | 390.0 mg, yield: 62.4% as a yellow solid. 1H NMR: (500 MHz, CDCl₃) δ |
| | ppm: 6.98-6.96 (m, 2H), 5.17-5.15 (m, 1H), 5.15-5.11 (m, 1H), 4.97-4.95 (m, |
| | 1H), 4.93-4.92 (m, 1H), 3.86 (s, 3H). |
| 69 | 2-bromo-6-(3-fluorooxetan-3-yl)-4-isopropoxypyridine |
| | 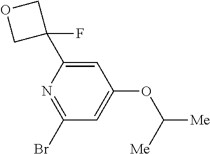 [SMILES: CC(C)Oc1cc(Br)nc(C2(F)COC2)c1] |
| | Alcohol: 3-(6-bromo-4-isopropoxypyridin-2-yl)oxetan-3-ol (Preparation 42) |
| | 320 mg, 47.4% yield as a yellow oil. 1H NMR: (400 MHz, CDCl₃) δ: ppm |
| | 6.92 (s, 2H), 5.17-5.15 (m, 1H), 5.11-5.09 (m, 1H), 4.97-4.95 (m, 1H), 4.91- |
| | 4.90 (m, 1H), 4.63-4.61 (m, 1H), 1.36 (d, J-6.0 Hz, 6H). |
| 70 | 2-bromo-6-(3-fluorooxetan-3-yl)-4-(2-methoxyethoxy)pyridine |
| | 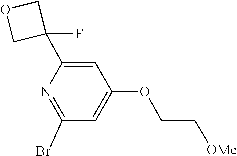 [SMILES: COCCOc1cc(Br)nc(C2(F)COC2)c1] |
| | Alcohol: 3-(6-bromo-4-(2-methoxyethoxy)pyridin-2-yl)oxetan-3-ol |
| | (Preparation 43) |
| | 328.9 mg, 70.8% yield as white solid. |
| | 1H NMR: (400 MHz, CDCl₃) δ ppm: 7.01 (d, J = 5.2 Hz, 2H), 5.17-5.09 (m, |
| | 2H), 4.97-4.90 (m, 2H), 4.19-4.17 (m, 2H), 3.76-3.74 (m, 2H), 3.44 (s, 3H). |
| 71 | 2-bromo-6-(2-fluoro-1-methoxypropan-2-yl)pyridine |
| | 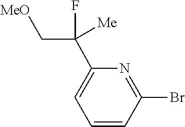 [SMILES: COCC(C)(F)c1cccc(Br)n1] |
| | Alcohol: 2-(6-bromopyridin-2-yl)-1-methoxypropan-2-ol (Preparation 58) |
| | 72.8 mg, 60.2% yield as colorless oil. 1H NMR: (500 MHz, CDCl₃) δ ppm: |
| | 7.59-7.54 (m, 2H), 7.38 (d, J = 6.0 Hz, 1H), 3.82-3.76 (m, 2H), 3.37 (s, 3H), |
| | 1.67 (d, J = 22.0 Hz, 3H). |
| 72 | 2-bromo-6-(3-fluorooxetan-3-yl)pyridine |
| | 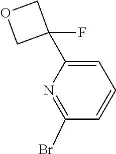 [SMILES: FC1(c2cccc(Br)n2)COC1] |
| | Alcohol: 3-(6-bromopyridin-2-yl)oxetan-3-ol (Preparation 38) |
| | 493.2 mg, 71.9% yeld as a white solid. ¹H NMR (400 MHz, CDCl₃ δ ppm: |
| | 7.62-7.58 (m, 1H), 7.49-7.45 (m, 2H), 5.11-5.19 (m, 2H), 4.94-5.01 (m, 2H). |

### Preparation 73: 3-(6-bromopyridin-2-yl)-3-methyldihydrofuran-2(3H)-one

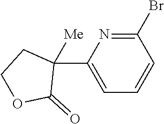 [SMILES: CC1(c2cccc(Br)n2)CCOC1=O]

To a solution of 2-bromo-6-fluoropyridine (1.5 g, 8.52 mmol) and 3-methyldihydrofuran-2(3H)-one (1.7 g, 17.05 mmol) in toluene (15 mL) was added LiHMDS (1 M, 25.6 mL) and the reaction stirred at 120° C. under microwave irradiation for 1.5 h. The reaction was quenched with water (10 mL), extracted with DCM (15 mL×3) and the combined organic phase was concentrated under reduced pressure. The residue was purified by column chromatography on silica gel (PE/EtOAc=10/1 to 5/1) to give 3-(6-bromopyridin-2-yl)-3-methyldihydrofuran-2(3H)-one (1.0 g, 45.8% yield) as a colorless oil. 1H NMR: (500 MHz, CDCl₃) δ ppm: 7.56-7.51 (m, 2H), 7.41-7.40 (m, 1H), 4.38-4.34 (m, 2H), 3.20-3.16 (m, 1H), 2.45-2.43 (m, 1H), 1.65 (s, 3H).

### Preparation 74: 2-(6-bromopyridin-2-yl)-2-methylbutane-1,4-diol

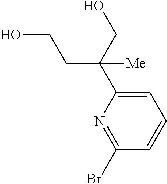 [SMILES: CC(CO)(CCO)c1cccc(Br)n1]

To a solution of 3-(6-bromopyridin-2-yl)-3-methyldihydrofuran-2(3H)-one (Preparation 73, 1.0 g, 3.90 mmol) in MeOH (10 mL) was added NaBH₄ (443.2 mg, 11.7 mmol) slowly at 0° C. over 30 mins and the reaction then stirred at 30° C. for 2 h. The reaction was concentrated under reduced pressure, diluted with brine (10 mL) and extracted with DCM (15 mL×3). The combined organic phase was concentrated in vacuo and the residue was purified by column chromatography on silica gel (PE/EtOAc=1/1 to 0/1) to give 2-(6-bromopyridin-2-yl)-2-methylbutane-1,4-diol (305 mg, 30% yield) as a colorless oil. 1H NMR (400 MHz, CDCl₃) δ ppm: 7.55 (t, J=8.0 Hz, 1H), 7.36 (d, J=8.0 Hz, 1H), 7.28-7.26 (m, 1H), 4.55 (br s, 1H), 4.05-4.02 (m, 1H), 3.70-3.62 (m, 2H), 3.50-3.47 (m, 1H), 2.13-2.08 (m, 1H), 2.02-1.96 (m, 1H), 1.25 (s, 3H).

### Preparation 75: 2-bromo-6-(3-methyltetrahydrofuran-3-yl)pyridine

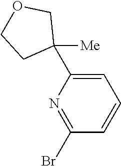 [SMILES: CC1(c2cccc(Br)n2)CCOC1]

To a solution of 2-(6-bromopyridin-2-yl)-2-methylbutane-1,4-diol (Preparation 74, 305 mg, 1.17 mmol) in THF (5.0 mL) was added NaH (93.8 mg, 2.35 mmol, 60% purity) at −30° C., the solution stirred for 15 mins, then TsCl (245.9 mg, 1.29 mmol) was added. The resulting mixture was stirred at 70° C. for 2 h, then quenched with water (1 mL) and concentrated in vacuo. The residue was purified by chromatography on silica gel (PE/EtOAc=5/1 to 3/1) to give 2-bromo-6-(3-methyltetrahydrofuran-3-yl)pyridine (20.0 mg, 7.0% yield) as a colorless oil. LCMS m/z=244.1 [M+H]⁺

### Preparation 76: 2-(3,6-dihydro-2H-pyran-4-yl)-6-fluoropyridine

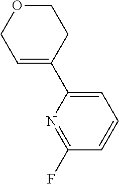 [SMILES: Fc1cccc(C2=CCOCC2)n1]

To a solution of 2-bromo-6-fluoropyridine (3.0 g, 17.05 mmol) in dioxane (40 mL) and H₂O (10 mL) was added 2-(3,6-dihydro-2H-pyran-4-yl)-4,4,5,5-tetramethyl-1,3,2-dioxaborolane (2.9 g, 14.21 mmol), Pd(dppf)Cl₂ (1.1 g, 1.42 mmol) and K₂CO₃ (3.9 g, 28.41 mmol) and the reaction mixture was stirred at 80° C. for 1 h under N₂. The mixture was poured into H₂O (40 mL), extracted with EtOAc (30 mL×3), the combined organic layer was dried over anhydrous Na₂SO₄, filtered and concentrated in vacuo. The residue was purified by chromatography on silica gel (PE/EtOAc=10/1 to 5/1) to give 2-(3,6-dihydro-2H-pyran-4-yl)-6-fluoropyridine (1.6 g, 60.9% yield) as colorless oil. ¹H NMR (500 MHz, CDCl₃) δ ppm: 7.73-7.72 (m, 1H), 7.23-7.21 (m, 1H), 6.80-6.78 (m, 2H), 4.38-4.36 (m, 2H), 3.94-3.92 (m, 2H), 2.60-2.58 (m, 2H).

### Preparation 77: 2-(3,7-dioxabicyclo[4.1.0]heptan-6-yl)-6-fluoropyridine

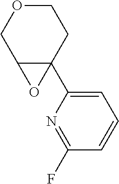 [SMILES: Fc1cccc(C23CCOCC2O3)n1]

To a solution of 2-(3,6-dihydro-2H-pyran-4-yl)-6-fluoropyridine (Preparation 76, 1.6 g, 8.37 mmol) in DCM (20 mL) was added 3-chlorobenzoperoxoic acid (1.7 g, 10.05 mmol) at 0° C. and the resulting mixture was stirred at 25° C. for 12 h. The reaction was poured into sat. Na₂CO₃ (30 mL), extracted with EtOAc (20 mL×3), the combined organic layer was dried over anhydrous Na₂SO₄, filtered and concentrated in vacuo. The residue was purified by chromatography on silica gel (PE/EtOAc=10/1 to 5/1) to give 2-(3,7-dioxabicyclo[4.1.0]heptan-6-yl)-6-fluoropyridine (2.4 g, crude) as a white solid. ¹H NMR (500 MHz, CDCl₃) δ ppm: 7.81-7.77 (m, 1H), 7.29-7.27 (m, 1H), 6.87-6.85 (m, 1H), 4.07-3.74 (m, 2H), 3.73-3.72 (m, 1H), 3.65-3.62 (m, 1H), 2.91-2.87 (m, 1H), 2.12-2.08 (m, 1H).

### Preparation 78: 3-(6-fluoropyridin-2-yl)tetrahydrofuran-3-carbaldehyde

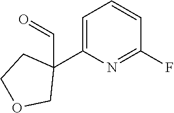 [SMILES: O=CC1(c2cccc(F)n2)CCOC1]

To a solution of 2-(3,7-dioxabicyclo[4.1.0]heptan-6-yl)-6-fluoropyridine (Preparation 77, 1.0 g, 5.12 mmol) in DCM (15 mL) was added BF₃·Et₂O (2.2 g, 15.37 mmol) slowly at 0° C. and the reaction was stirred at 20° C. for 12 h. The mixture was poured into sat. Na₂CO₃ (20 mL), extracted with EtOAc (20 mL×3), the combined organic layer was dried over anhydrous Na₂SO₄, filtered and concentrated in vacuo. The residue was purified by chromatography on silica gel (PE/EtOAc=5/1 to 3/1) to give 3-(6-fluoropyridin-2-yl)tetrahydrofuran-3-carbaldehyde (621.9 mg, 62.2% yield) as a white solid. ¹H NMR: (400 MHz, CDCl₃) δ ppm: 9.75 (s, 1H), 7.84-7.78 (m, 1H), 7.13-7.10 (m, 1H), 6.90-6.88 (m, 1H), 4.55-4.53 (m, 1H), 4.09-4.06 (m, 1H), 3.99-3.97 (m, 2H), 2.77-2.72 (m, 1H), 2.48-2.43 (m, 1H).

### Preparation 79: (3-(6-fluoropyridin-2-yl)tetrahydrofuran-3-yl)methanol

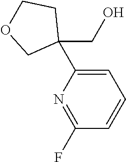 [SMILES: OCC1(c2cccc(F)n2)CCOC1]

To a mixture of 3-(6-fluoropyridin-2-yl)tetrahydrofuran-3-carbaldehyde (Preparation 78, 400 mg, 2.05 mmol) in MeOH (3 mL) was added NaBH₄ (116.3 mg, 3.07 mmol) at 0° C. and the reaction then stirred at 25° C. for 1 h. The mixture was quenched with NH₄Cl aq. (10 mL), poured into H₂O (10 mL), extracted with EtOAc (10 mL×3), the combined organic layer was dried over anhydrous Na₂SO₄, filtered and concentrated in vacuo. The residue was purified by chromatography (PE/EtOAc=5/1 to 3/1) on silica gel to give (3-(6-fluoropyridin-2-yl)tetrahydrofuran-3-yl)methanol (310.3 mg, 76.8% yield) as colorless oil. ¹H NMR: (500 MHz, CDCl₃) δ ppm: 7.81-7.76 (m, 1H), 7.24-7.22 (m, 1H), 6.83.-6.81 (m, 1H), 4.05-3.86 (m, 6H), 3.15 (br s, 1H), 2.27-2.19 (m, 2H).

### Preparation 80: 2-fluoro-6-(3-(fluoromethyl)tetrahydrofuran-3-yl)pyridine

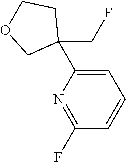 [SMILES: FCC1(c2cccc(F)n2)CCOC1]

To a solution of (3-(6-fluoropyridin-2-yl)tetrahydrofuran-3-yl)methanol (Preparation 79, 310 mg, 1.57 mmol) in DCM (5.0 mL) was added Deoxofluor (365.5 mg, 1.65 mmol) slowly at 0° C. and the reaction was stirred at 20° C. for 4 h under N₂. The mixture was concentrated under reduced pressure and was purified by silica gel column chromatography (PE/EtOAc=5/1 to 3/1) to give 2-fluoro-6-(3-(fluoromethyl)tetrahydrofuran-3-yl)pyridine (72.9 mg, 23.3% yield) as a white solid. 1H NMR (500 MHz, CDCl₃) δ ppm: 7.79-7.74 (m, 1H), 7.21-7.18 (m, 1H), 6.84-6.81 (m, 1H), 4.62 (d, J=47.5 Hz, 2H), 4.12-3.96 (m, 4H), 2.40-2.37 (m, 1H), 2.23-2.20 (m, 1H).

### Preparation 81: 2-chloro-6-(3,6-dihydro-2H-pyran-4-yl)pyridine

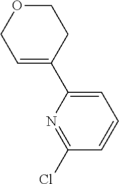 [SMILES: Clc1cccc(C2=CCOCC2)n1]

2-Chloro-6-(3,6-dihydro-2H-pyran-4-yl)pyridine was obtained as a yellow oil, 373 g, 62% yield, from 2-bromo-6-chloropyridine, following a similar procedure to that described in Preparation 76. LCMS m/z=196 [M+H]⁺

### Preparation 82: 2-(3,7-dioxabicyclo[4.1.0]heptan-6-yl)-6-chloropyridine

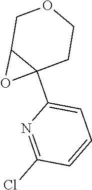 [SMILES: Clc1cccc(C23CCOCC2O3)n1]

2-(3,7-Dioxabicyclo[4.1.0]heptan-6-yl)-6-chloropyridine was obtained as a clear oil, 6.6 g, 61% yield, from 2-chloro-6-(3,6-dihydro-2H-pyran-4-yl)pyridine (Preparation 81), following a similar procedure to that described in Preparation 77. LCMS m/z=212 [M+H]⁺

### Preparation 83: 3-(6-chloropyridin-2-yl)tetrahydrofuran-3-carbaldehyde

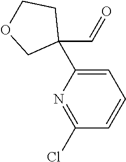 [SMILES: O=CC1(c2cccc(Cl)n2)CCOC1]

Sc(OTf)₃ (0.46 g, 0.94 mmol) was added to a solution of 2-(3,7-dioxabicyclo[4.1.0]heptan-6-yl)-6-chloropyridine (Preparation 82, 6.60 g, 31.3 mmol) in dry dioxane (100 mL) and the reaction was stirred at 95° C. for 30 min under N₂. The reaction mixture was cooled to rt and concentrated under vacuum. The residue was purified by column chromatography on silica gel (10-30% EtOAc in hexane) to give, 3-(6-chloropyridin-2-yl)tetrahydrofuran-3-carbaldehyde (5.40 g, 82% yield) as clear oil. LCMS m/z=212 [M+H]⁺

### Preparation 84: 2-chloro-6-(3-vinyltetrahydrofuran-3-yl)pyridine

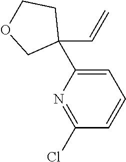

Under N₂, Ph₃PCH₃Br (9.68 g, 28.0 mmol) was added in portions to a solution of t-BuOK (3.13 g, 27.97 mmol) in dry THF (80 mL) at 5-10 0° C. and the resulting yellow suspension was stirred at rt for 1 h, then cooled to 0° C. 3-(6-Chloropyridin-2-yl)tetrahydrofuran-3-carbaldehyde (Preparation 83, 4.40 g, 20.85 mmol) in THF (20 ml) was added dropwise at 0° C. and the reaction was stirred for 1 h at 0-10° C. The mixture was quenched with NH₄Cl aq. (50 mL) and extracted with EtOAc (2×100 mL). The combined organic layer was washed with water then brine, dried over Na₂SO₄ and concentrated. The crude was purified by silica gel chromatography eluting with 10-20% EtOAc in hexane to give 2-chloro-6-(3-vinyltetrahydrofuran-3-yl)pyridine (3.90 g, 89% yield). LCMS m/z=210 [M+H]⁺

### Preparation 85: 2-chloro-6-(3-ethyltetrahydrofuran-3-yl)pyridine

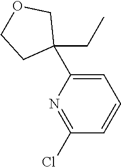

A mixture of 2-chloro-6-(3-vinyltetrahydrofuran-3-yl)pyridine (Preparation 84, 3.90 g, 18.66 mmol) and 10% Pd/C (0.50 g) in EtOAc was stirred for 1 h under H₂ (balloon). Pd/C was filtered off through Celite®. The filtrate was concentrated in vacuo and the residue purified by silica gel chromatography eluting with 10-20% EtOAc in hexane to give 2-chloro-6-(3-ethyltetrahydrofuran-3-yl)pyridine (3.50 g, 89% yield). LCMS m/z=212 [M+H]⁺

### Preparation 86: 2-(3-(difluoromethyl)tetrahydrofuran-3-yl)-6-fluoropyridine

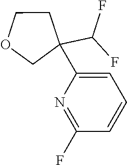 [SMILES: Fc1cccc(C2(C(F)F)CCOC2)n1]

To a solution of 3-(6-fluoropyridin-2-yl)tetrahydrofuran-3-carbaldehyde (Preparation 78, 200 mg, 1.02 mmol) in DCM (5 mL) was added Deoxofluor (566.7 mg, 2.56 mmol) slowly at 0° C. and the reaction was stirred at 20° C. for 12 h under N₂. The mixture was concentrated under reduced pressure and purified by column chromatography on silica gel (PE/EtOAc=10/1 to 5/1) to give 2-(3-(difluoromethyl)tetrahydrofuran-3-yl)-6-fluoropyridine (56.1 mg, 25.2% yield) as colorless oil. ¹H NMR (500 MHz, CDCl₃) S ppm: 7.82-7.78 (m, 1H), 7.26-7.24 (m, 1H), 6.89-6.86 (m, 1H), 6.16 (t, J=56.0 Hz, 1H), 4.31-4.29 (m, 1H), 4.18-4.16 (m, 1H), 4.04-3.92 (m, 2H), 2.53-2.45 (m, 2H).

### Preparation 87: 3-(6-bromopyridin-2-yl)tetrahydrofuran-3-carbonitrile

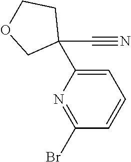 [SMILES: N#CC1(c2cccc(Br)n2)CCOC1]

To NaH (81.2 mg, 2.03 mmol, 60% purity) in DMF (40 mL) was added 2-(6-bromopyridin-2-yl)acetonitrile (400 mg, 2.03 mmol) and 1-chloro-2-(chloromethoxy)ethane (261.9 mg, 2.03 mmol) in DMF (10 mL) at −10° C. and the reaction stirred at −10° C. for 30 mins. The mixture was quenched with NH₄Cl (sat. 10 mL), poured into H₂O (20 mL) and extracted with EtOAc (3×20 mL). The combined organic layer was dried over anhydrous Na₂SO₄, filtered and concentrated in vacuo. The residue was purified by silica gel column chromatography (PE/EtOAc=15/1 to 3/1) to give 3-(6-bromopyridin-2-yl)tetrahydrofuran-3-carbonitrile (212.7 mg, 41.4% yield) as yellow oil. ¹H NMR: (400 MHz, CDCl₃) δ ppm: 7.66-7.60 (m, 2H), 7.49-7.45 (m, 1H), 4.41-4.38 (m, 1H), 4.21-4.18 (m, 2H), 4.15-4.12 (m, 1H), 2.86-2.80 (m, 1H), 2.70-2.64 (m, 1H).

### Preparation 88: 1-(6-chloropyridin-2-yl)pyrrolidin-2-one

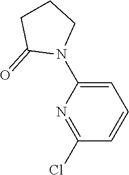 [SMILES: O=C1CCCN1c1cccc(Cl)n1]

Under N₂, a suspension of 2-bromo-6-chloropyridine (2.0 g, 10.39 mmol), pyrrolidin-2-one (884.4 mg, 10.39 mmol), Pd₂(dba)₃ (475.9 mg, 0.52 mmol), Xantphos (601.4 mg, 1.04 mmol) and K₂CO₃ (2.87 g, 20.8 mmol) in dioxane (20 mL) was stirred at 100° C. for 16 h. The reaction was concentrated in vacuo, the residue was diluted with water (100 mL) and extracted with EtOAc (100 mL×2). The combined organic layer was washed with water (100 mL) and brine (100 mL), dried over Na₂SO₄, filtered and concentrated in vacuo. The residue was purified by silica gel chromatography (EtOAc=100%) to afford 1-(6-chloropyridin-2-yl)pyrrolidin-2-one (1.10 g, 53.8% yield) as a white solid. LCMS m/z=197.1 [M+H]⁺

### Preparation 89: 2-chloro-6-(3-methoxytetrahydrofuran-3-yl)-4-(4,4,5,5-tetramethyl-1,3,2-dioxaborolan-2-yl)pyridine

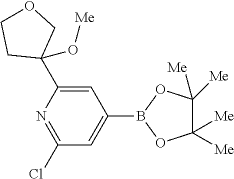 [SMILES: COC1(c2cc(B3OC(C)(C)C(C)(C)O3)cc(Cl)n2)CCOC1]

2-Chloro-6-(3-methoxytetrahydrofuran-3-yl)pyridine (Preparation 61, 11.9 g, 55.8 mmol) was dissolved in hexane (170 mL), degassed with N₂, then 4,4′-di-tert-butyl-2,2′-bipyridine (298 mg, 1.12 mmol), bis(1,5-cyclooctadiene) diiridium (I) dichloride (381 mg, 0.56 mmol), and 4,4,4′,4′, 5,5,5′, 5′-octamethyl-2,2′-bi (1,3,2-dioxaborolane) (16.7 g, 65.6 mmol) was added. The reaction was heated at 75° C. for 1 h under N₂ and the mixture was cooled to rt. The solid formed was collected by filtration, washed with cold hexane and dried to give 2-chloro-6-(3-methoxytetrahydrofuran-3-yl)-4-(4,4,5,5-tetramethyl-1,3,2-dioxaborolan-2-yl)pyridine, (6.0 g, 32% yield).

### Preparation 90: 2-chloro-6-(3-methoxytetrahydrofuran-3-yl)pyridin-4-ol

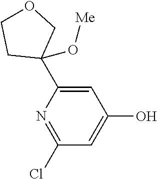 [SMILES: COC1(c2cc(O)cc(Cl)n2)CCOC1]

Oxone (9.56 g, 15.6 mmol) in water (50 mL) was added dropwise to a mixture of 2-chloro-6-(3-methoxytetrahydrofuran-3-yl)-4-(4,4,5,5-tetramethyl-1,3,2-dioxaborolan-2-yl)pyridine (Preparation 89, 4.4 g, 13.0 mmol) in THF (50 mL) at 0° C., then the reaction was stirred at this temperature for 1 h. The mixture was quenched with Na₂SO₃ (saturated solution), extracted with EtOAc (100 mL×2), the combined organic layer was dried over anhydrous Na₂SO₄, filtered and concentrated to give 2-chloro-6-(3-methoxytetrahydrofuran-3-yl)pyridin-4-ol (3.3 g, crude) as a brown oil. LCMS m/z=229.9 [M+H]⁺

### Preparation 91: 2-chloro-4-methoxy-6-(3-methoxytetrahydrofuran-3-yl)pyridine

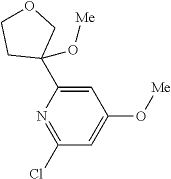 [SMILES: COc1cc(Cl)nc(C2(OC)CCOC2)c1]

MeI (166.8 mg, 1.18 mmol) was added to a mixture of 2-chloro-6-(3-methoxytetrahydrofuran-3-yl)pyridin-4-ol (Preparation 90, 180 mg, 0.784 mmol) and Cs₂CO₃ (383 mg, 1.18 mmol) in DMF (10 mL) under N₂ and the reaction was stirred at 40° C. for 0.5 h. The mixture was diluted with water (15 mL) and extracted with EtOAc (10 mL×3). The combined organic layers were washed with H₂O (10 mL×3) and brine (20 mL), dried over Na₂SO₄, filtered and concentrated under reduced pressure to give 2-chloro-4-methoxy-6-(3-methoxytetrahydrofuran-3-yl)pyridine (160 mg, 84% yield) as yellow oil, which was used in the next step without further purification. 1H NMR (400 MHz, CDCl₃) δ ppm 7.00 (d, J=2.0 Hz, 1H), 6.76 (d, J=2.0 Hz, 1H), 4.17-4.02 (m, 3H), 3.98-3.95 (m, 1H), 3.88 (s, 3H), 3.23 (s, 3H), 2.68-2.60 (m, 1H), 2.35-2.30 (m, 1H).

### Preparation 92: 2-chloro-6-(3-methoxytetrahydrofuran-3-yl)-4-(oxetan-3-yloxy)pyridine

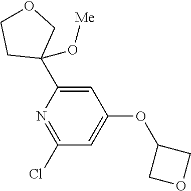 [SMILES: COC1(c2cc(OC3COC3)cc(Cl)n2)CCOC1]

2-Chloro-6-(3-methoxytetrahydrofuran-3-yl)-4-(oxetan-3-yloxy)pyridine was obtained, 120 mg, crude, from 2-chloro-6-(3-methoxytetrahydrofuran-3-yl)pyridin-4-ol (Preparation 90) and 3-iodooxetane, following a similar procedure to that described in Preparation 91. LCMS m/z=286.1 [M+H]⁺

### Preparation 93: 2-chloro-6-(3-methoxytetrahydrofuran-3-yl)-4-(oxetan-2-ylmethoxy)pyridine

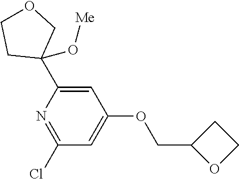 [SMILES: COC1(c2cc(OCC3CCO3)cc(Cl)n2)CCOC1]

A solution of oxetan-2-ylmethyl 4-methylbenzenesulfonate (500 mg, 2.18 mmol), 2-chloro-6-(3-methoxytetrahydrofuran-3-yl)pyridin-4-ol (Preparation 90, 633.0 mg, 2.61 mmol) and Cs₂CO₃ (1.8 g, 5.44 mmol) in DMF (5 mL) was stirred at 70° C. for 18 h under N₂. The mixture was diluted with water (10 mL) and extracted with DCM (15 mL×3). The combined organic layers were washed with brine (20 mL), filtered and concentrated in vacuo. The crude product was purified by column chromatography on silica gel (PE/EtOAc=15/1 to 1/1) to give 2-chloro-6-(3-methoxytetrahydrofuran-3-yl)-4-(oxetan-2-ylmethoxy)pyridine (290 mg, 44.4% yield) as colorless oil. ¹H NMR: (400 MHz, CDCl₃) δ ppm: 7.07 (d, J=1.6 Hz, 1H), 6.83-6.82 (m, 1H), 5.16-5.13 (m, 1H), 4.75-4.65 (m, 2H), 4.20-3.94 (m, 6H), 3.21 (s, 3H), 2.81-2.59 (m, 3H), 2.32-2.30 (m, 1H).

### Preparation 94: 2-chloro-6-(3-methoxytetrahydrofuran-3-yl)-4-(oxetan-3-ylmethoxy)pyridine

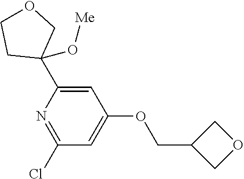 [SMILES: COC1(c2cc(OCC3COC3)cc(Cl)n2)CCOC1]

2-Chloro-6-(3-methoxytetrahydrofuran-3-yl)-4-(oxetan-3-ylmethoxy)pyridine was obtained as a colorless oil (310 mg, 79.2%) from oxetan-3-ylmethyl 4-methylbenzenesulfonate and 2-chloro-6-(3-methoxytetrahydrofuran-3-yl)pyridin-4-ol (Preparation 90), following the procedure described in Preparation 93. 1H NMR: (400 MHz, CDCl₃) δ ppm: 7.02-7.00 (m, 1H), 6.78 (d, J=2.0 Hz, 1H), 4.92-4.88 (m, 2H), 4.57-4.53 (m, 2H), 4.28-4.26 (m, 2H), 4.11-4.09 (m, 3H), 4.08-3.94 (m, 1H), 3.47-3.43 (m, 1H), 3.23 (s, 3H), 2.66-2.60 (m, 1H), 2.34-2.32 (m, 1H).

### Preparation 95: 2-chloro-4-(3-(difluoromethyl)cyclobutoxy)-6-(3-methoxytetrahydrofuran-3-yl)pyridine

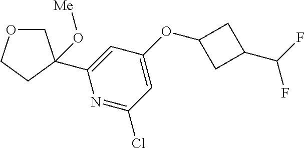 [SMILES: COC1(c2cc(OC3CC(C(F)F)C3)cc(Cl)n2)CCOC1]

2-Chloro-4-(3-(difluoromethyl)cyclobutoxy)-6-(3-methoxytetrahydrofuran-3-yl)pyridine was obtained as a colorless oil (305 mg, 43.1% yield) from 3-(difluoromethyl)cyclobutyl 4-methylbenzenesulfonate and 2-chloro-6-(3-methoxytetrahydrofuran-3-yl)pyridin-4-ol (Preparation 90), following a similar procedure to that described in Preparation 93. LCMS m/z=334.3 [M+H]⁺

### Preparation 96: 3-(benzyloxy)cyclobutyl 4-methylbenzenesulfonate

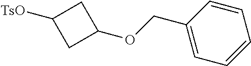 [SMILES: Cc1ccc(S(=O)(=O)OC2CC(OCc3ccccc3)C2)cc1]

To a solution of 3-(benzyloxy)cyclobutan-1-ol (1.0 g, 5.61 mmol) in DCM (15.0 mL) was added TsCl (1.3 g, 6.73 mmol), DMAP (137.1 mg, 1.12 mmol) and TEA (1.1 g, 11.22 mmol) and the reaction mixture was stirred at 20° C. for 3 h. The mixture was concentrated in vacuo and the residue was purified by column chromatography on silica gel (PE/EtOAc=5/1) to give 3-(benzyloxy)cyclobutyl 4-methylbenzenesulfonate (1.6 g, 84.7% yield) as colorless oil. ¹H NMR: (400 MHz, CDCl₃) δ ppm: 7.78-7.75 (m, 2H), 7.34-7.27 (m, 7H), 4.49-4.41 (m, 1H), 4.36 (s, 2H), 3.66-3.58 (m, 1H), 2.62-2.58 (m, 2H), 2.44 (s, 3H), 2.18-2.14 (m, 2H).

### Preparation 97: 4-(3-(benzyloxy)cyclobutoxy)-2-chloro-6-(3-methoxytetrahydrofuran-3-yl)pyridine

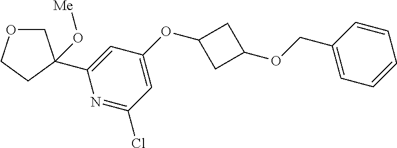 [SMILES: COC1(c2cc(OC3CC(OCc4ccccc4)C3)cc(Cl)n2)CCOC1]

4-(3-(Benzyloxy)cyclobutoxy)-2-chloro-6-(3-methoxytetrahydrofuran-3-yl)pyridine was obtained as a colorless oil (807 mg, 95%) from 3-(benzyloxy)cyclobutyl 4-methylbenzenesulfonate (Preparation 96) and 2-chloro-6-(3-methoxytetrahydrofuran-3-yl)pyridin-4-ol (Preparation 90), following a similar procedure to that described in Preparation 93. 1H NMR (400 MHz, CDCl₃) δ ppm: 7.37-7.27 (m, 5H), 6.88 (d, J=2.0 Hz, 1H), 6.61 (d, J=1.6 Hz, 1H), 4.92-4.89 (m, 1H), 4.34 (s, 2H), 4.12-4.10 (m, 1H), 4.09-4.06 (m, 3H), 3.96-3.93 (m, 1H), 3.20 (s, 3H), 2.64-2.58 (m, 3H), 2.56-2.45 (m, 2H), 2.44-2.29 (m, 1H).

### Preparation 98: 2-chloro-4-(3,3-difluorocyclobutoxy)-6-(3-methoxytetrahydrofuran-3-yl)pyridine

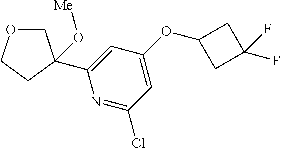 [SMILES: COC1(c2cc(OC3CC(F)(F)C3)cc(Cl)n2)CCOC1]

To a solution of 3,3-difluorocyclobutyl 4-methylbenzenesulfonate (100 mg, 0.435 mmol) in DMF (3 mL) was added 2-chloro-6-(3-methoxytetrahydrofuran-3-yl)pyridin-4-ol (Preparation 90), 114.2 mg, 0.435 mmol) and Cs₂CO₃ (283.7 mg, 0.871 mmol) and the reaction was stirred at 80° C. for 40 h. The mixture was purified by Prep-HPLC-K (gradient: 40-70% MeCN) to give 2-chloro-4-(3,3-difluorocyclobutoxy)-6-(3-methoxytetrahydrofuran-3-yl)pyridine (50 mg, 35.9% yield) as yellow gum. 1H NMR: (500 MHz, MeOH-d₄) δ ppm: 7.03 (d, J=2.5 Hz, 1H), 6.90 (d, J=2.5 Hz, 1H), 5.11-4.92 (m, 5H), 3.23-3.19 (m, 5H), 2.83-2.80 (m, 1H), 2.80-2.75 (m, 3H).

### Preparation 99: 2-chloro-4-ethoxy-6-(3-methoxytetrahydrofuran-3-yl)pyridine

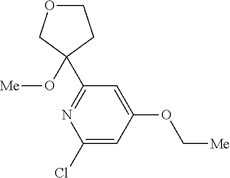 [SMILES: CCOc1cc(Cl)nc(C2(OC)CCOC2)c1]

To a solution of 2-chloro-6-(3-methoxytetrahydrofuran-3-yl)pyridin-4-ol (Preparation 90, 100 mg, 0.435 mmol) in DMF (3 mL) was added K₂CO₃ (120.4 mg, 0.871 mmol) and iodoethane (101.9 mg, 0.653 mmol) and the reaction was stirred at 50° C. for 3 h. The reaction was concentrated under reduced pressure and the residue was purified by chromatography on silica gel (PE/EtOAc=1/1) to give 2-chloro-4-ethoxy-6-(3-methoxytetrahydrofuran-3-yl)pyridine (105 mg, 93.6% yield) as a colorless oil. LCMS m/z=258.1 [M+H]⁺

### Preparation 100: 2-chloro-4-isopropoxy-6-(3-methoxytetrahydrofuran-3-yl)pyridine

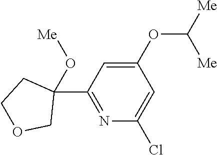 [SMILES: COC1(c2cc(OC(C)C)cc(Cl)n2)CCOC1]

2-Chloro-4-isopropoxy-6-(3-methoxytetrahydrofuran-3-yl)pyridine was obtained as a colorless oil, 95 mg, 80% yield, from 2-chloro-6-(3-methoxytetrahydrofuran-3-yl)pyridin-4-ol (Preparation 90) and isopropanol, following the procedure described in Preparation 99. ¹H NMR: (500 MHz, CDCl₃) δ: ppm 6.97-6.92 (m, 1H), 6.71-6.68 (m, 1H), 4.65-4.62 (m, 1H), 4.13-4.05 (m, 3H), 3.96-3.93 (m, 1H), 3.21 (s, 3H), 2.66-2.59 (m, 1H), 2.33-2.30 (m, 1H), 1.37 (d, J=6.0 Hz, 6H).

### Preparation 101: 2-chloro-6-(3-methoxytetrahydrofuran-3-yl)-4-(2,2,2-trifluoroethoxy)pyridine

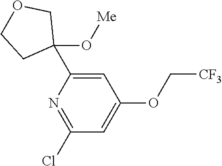 [SMILES: COC1(c2cc(OCC(F)(F)F)cc(Cl)n2)CCOC1]

To a solution of 2-chloro-6-(3-methoxytetrahydrofuran-3-yl)pyridin-4-ol (Preparation 90, 200 mg, 0.871 mmol) and 2,2,2-trifluoroethyl trifluoromethanesulfonate (202.1 mg, 0.871 mmol) in DMF (10.0 mL) was added K₂CO₃ (361.0 mg, 2.61 mmol) and the reaction stirred at 70° C. for 8 h. The mixture was diluted with H₂O (10 mL), extracted with EtOAc (10 mL×3), the combined organic layer was dried over anhydrous Na₂SO₄, filtered and concentrated in vacuo. The residue was purified by column chromatography on silica gel (PE/EtOAc=15/1 to 3/1) to give 2-chloro-6-(3-methoxytetrahydrofuran-3-yl)-4-(2,2,2-trifluoroethoxy)pyridine (130 mg, 47.9% yield) and a further 130 mg of impure product. LCMS m/z=312.1 [M+H]⁺

### Preparation 102: 3-((2-chloro-6-(3-methoxytetrahydrofuran-3-yl)pyridin-4-yl)oxy)cyclobutan-1-ol

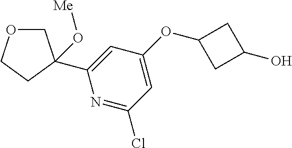 [SMILES: COC1(c2cc(OC3CC(O)C3)cc(Cl)n2)CCOC1]

A solution of 4-(3-(benzyloxy)cyclobutoxy)-2-chloro-6-(3-methoxytetrahydrofuran-3-yl)pyridine (Preparation 97, 200 mg, 0.513 mmol) in TFA (2.9 g, 26.1 mmol) was stirred at 100° C. for 4 h. The mixture was purified by Prep-HPLC-K (gradient: 24-54% MeCN) to give 3-((2-chloro-6-(3-methoxytetrahydrofuran-3-yl)pyridin-4-yl)oxy)cyclobutan-1-ol (105 mg, 68.3% yield) as a white solid. ¹H NMR: (400 MHz, CDCl₃) δ ppm: 6.89 (d, J=2.0 Hz, 1H), 6.61 (d, J=2.0 Hz, 1H), 4.96-4.92 (m, 1H), 4.66-4.65 (m, 1H), 4.12-4.06 (m, 3H), 3.96-3.93 (m, 1H), 3.21 (s, 3H), 2.63-2.47 (m, 5H), 2.46-2.30 (m, 1H).

### Preparation 103: 2-chloro-4-(3-methoxycyclobutoxy)-6-(3-methoxytetrahydrofuran-3-yl)pyridine

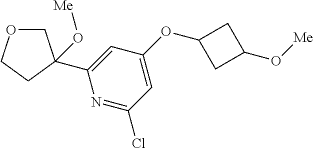 [SMILES: COC1CC(Oc2cc(Cl)nc(C3(OC)CCOC3)c2)C1]

2-Chloro-4-(3-methoxycyclobutoxy)-6-(3-methoxytetrahydrofuran-3-yl)pyridine was obtained (85.2 mg, 81.4% yield) as a colorless oil, from 3-((2-chloro-6-(3-methoxytetrahydrofuran-3-yl)pyridin-4-yl)oxy)cyclobutan-1-ol (Preparation 102) and CH₃I, following a similar procedure to that described in Preparation 54. ¹H NMR: (400 MHz, CDCl₃) δ ppm: 6.89 (d, J=2.0 Hz, 1H), 6.61 (d, J=2.0 Hz, 1H), 4.90-4.88 (m, 1H), 4.14-4.05 (m, 4H), 3.96-3.93 (m, 1H), 3.28 (s, 3H), 3.21 (s, 3H), 2.61-2.49 (m, 1H), 2.48-2.42 (m, 4H), 2.32-2.27 (m, 1H).

### Preparation 104: 4-(3-(benzyloxy)cyclobutoxy)-2,6-dichloropyridine

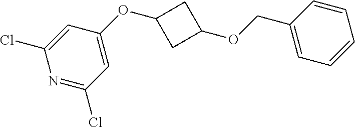 [SMILES: Clc1cc(OC2CC(OCc3ccccc3)C2)cc(Cl)n1]

To a solution of 3-(benzyloxy)cyclobutyl 4-methylbenzenesulfonate (Preparation 96, 4 g, 12.03 mmol) in DMF (40 mL) was added 2,6-dichloropyridin-4-ol (2.0 g, 12.0 mmol) and K₂CO₃ (3.33 g, 24.1 mmol) and the reaction was stirred at 70° C. for 16 h. The mixture was concentrated and the residue was purified by chromatography on silica gel (PE/EtOAc=3/1) to give 4-(3-(benzyloxy)cyclobutoxy)-2,6-dichloropyridine (3.4 g, 85.9% yield) as a yellow oil. 1H NMR: (400 MHz, CDCl₃) δ ppm: 7.39-7.29 (m, 5H), 6.68-6.66 (m, 2H), 4.89-4.86 (m, 1H), 4.45 (s, 2H), 4.34-4.31 (m, 1H), 2.60-2.52 (m, 2H), 2.47-2.43 (m, 2H).

### Preparation 105: 4-(3-(benzyloxy)cyclobutoxy)-2-chloro-6-(furan-3-yl)pyridine

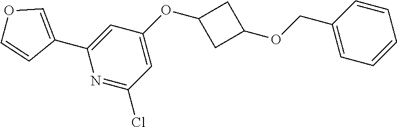 [SMILES: Clc1cc(OC2CC(OCc3ccccc3)C2)cc(-c2ccoc2)n1]

To a solution of 4-(3-(benzyloxy)cyclobutoxy)-2,6-dichloropyridine (Preparation 104, 3.4 g, 10.33 mmol) in dioxane (30 mL) and H₂O (6 mL) was added furan-3-ylboronic acid, K₂CO₃ (2.9 g, 20.67 mmol) and Pd(dppf)Cl₂ (756.1 mg, 1.03 mmol) and the reaction was stirred at 100° C. for 16 h under N₂. The mixture was concentrated and was purified by prep-HPLC-B (Gradient=62-92% MeCN) to give 4-(3-(benzyloxy)cyclobutoxy)-2-chloro-6-(furan-3-yl)pyridine (1.6 g, 43.0% yield) as a white oil. ¹H NMR: (400 MHz, CDCl₃) δ ppm: 8.03 (s, 1H), 7.47 (s, 1H), 7.39-7.31 (m, 5H), 6.80-6.77 (m, 1H), 6.66 (s, 1H), 6.57 (d, J=2.0 Hz, 1H), 4.95-4.91 (m, 1H), 4.46-4.45 (m, 2H), 4.36-4.33 (m, 1H), 2.57-2.54 (m, 2H), 2.48-2.41 (m, 2H).

### Preparation 106: 3-((2-chloro-6-(furan-3-yl)pyridin-4-yl)oxy)cyclobutan-1-ol

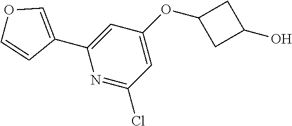 [SMILES: OC1CC(Oc2cc(Cl)nc(-c3ccoc3)c2)C1]

A solution of 4-(3-(benzyloxy)cyclobutoxy)-2-chloro-6-(furan-3-yl)pyridine (Preparation 105, 1.5 g, 4.30 mmol) in TFA (20 mL) was stirred at 100° C. for 5 h. The mixture was concentrated and the residue was purified by chromatography on silica gel (PE/EtOAc=10/1) to give 3-((2-chloro-6-(furan-3-yl)pyridin-4-yl)oxy)cyclobutan-1-ol (580 mg, 50.8% yield) as a yellow oil. ¹H NMR: (400 MHz, CDCl₃) δ ppm: 8.06 (s, 1H), 7.47 (d, J=2.0 Hz, 1H), 6.81-6.79 (m, 1H), 6.68 (s, 1H), 6.55 (d, J=1.6 Hz, 1H), 5.50-5.44 (m, 1H), 5.04-4.97 (m, 1H), 1.85 (br s, 1H), 1.30-1.24 (m, 2H), 0.90-0.85 (m, 2H).

### Preparation 107: 2-chloro-6-(furan-3-yl)-4-(3-methoxycyclobutoxy)pyridine

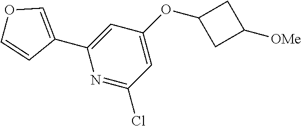 [SMILES: COC1CC(Oc2cc(Cl)nc(-c3ccoc3)c2)C1]

NaH (159.6 mg, 3.99 mmol, 60% purity) was added to a mixture of 3-((2-chloro-6-(furan-3-yl)pyridin-4-yl)oxy)cyclobutan-1-ol (Preparation 106, 530 mg, 1.99 mmol) in THF (6.0 mL) and the solution stirred for 30 mins at 25° C. MeI (566.3 mg, 3.99 mmol) was added and the reaction was stirred at 25° C. for 18 h. The reaction was concentrated in vacuo, diluted with H₂O (20 mL) and extracted with EtOAc (20 mL×3). The combined organic phase was washed with brine (20 mL×2), dried over Na₂SO₄ and filtered. The mixture was concentrated and the residue was purified by chromatography on silica gel (PE/EtOAc=3/1) to give 2-chloro-6-(furan-3-yl)-4-(3-methoxycyclobutoxy)pyridine (325 mg, 58.5% yield) as a yellow oil.

### Preparation 108:4-(3-(benzyloxy)cyclobutoxy)-2-chloropyridine

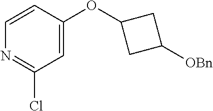 [SMILES: Clc1cc(OC2CC(OCc3ccccc3)C2)ccn1]

To a solution of 2-chloropyridin-4-ol (5.0 g, 38.6 mmol) and 3-(benzyloxy)cyclobutyl 4-methylbenzenesulfonate (Preparation 96, 12.8 g, 38.60 mmol) in DMF (70 mL) was added Cs₂CO₃ (37.7 g, 115.8 mmol) and the reaction was stirred at 100° C. for 6 h. The cooled mixture was concentrated under reduced pressure, the residue was treated with H₂O (50 mL) and extracted with EtOAc (20 mL×3). The combined organic phase was washed with brine (50 mL), dried over Na₂SO₄, filtered and the filtrate concentrated in vacuo. The crude was purified by column chromatography (PE/EtOAc=3/1 to 1/1) on silica gel to give 4-(3-(benzyloxy)cyclobutoxy)-2-chloropyridine (9.8 g, 87.6% yield) as yellow oil. LCMS m/z=290.7 [M+H]⁺

### Preparation 109: 1-(4-(3-(benzyloxy)cyclobutoxy)-6-chloropyridin-2-yl)ethan-1-one

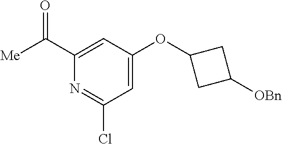 [SMILES: CC(=O)c1cc(OC2CC(OCc3ccccc3)C2)cc(Cl)n1]

To a solution of 4-(3-(benzyloxy)cyclobutoxy)-2-chloropyridine (Preparation 108, 10.8 g, 37.27 mmol) in MeCN (120 mL) was added acetaldehyde (16.4 g, 372.7 mmol), TBHP (6.7 g, 74.55 mmol), TFA (4.7 g, 41 mmol) and FeSO₄ (10.4 g, 37.27 mmol) and the reaction was stirred at 80° C. for 16 h under N₂. The mixture was concentrated under reduced pressure to give the residue, which was treated with H₂O (150 mL) and extracted with EtOAc (100 mL×3). The combined organic phase was washed with brine (200 mL), dried over Na₂SO₄, filtered and concentrated in vacuo. The crude was purified by column chromatography (PE/EtOAc=5/1 to 3/1) on silica gel to give 1-(4-(3-(benzyloxy)cyclobutoxy)-6-chloropyridin-2-yl)ethan-1-one (640 mg, 5.18% yield) as yellow oil and 4-(3-(benzyloxy)cyclobutoxy)-2-chloropyridine (8.6 g, 79.6% yield) as light yellow oil. LCMS m/z=331.7 [M+H]⁺

### Preparation 110: 4-(3-(benzyloxy)cyclobutoxy)-2-chloro-6-(1,1-difluoroethyl)pyridine

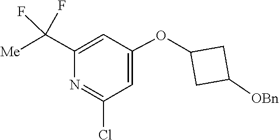 [SMILES: CC(F)(F)c1cc(OC2CC(OCc3ccccc3)C2)cc(Cl)n1]

To a solution of 1-(4-(3-(benzyloxy)cyclobutoxy)-6-chloropyridin-2-yl)ethan-1-one (Preparation 109, 600 mg, 1.81 mmol) in DCM (10 mL) was added DAST (7.3 g, 45.41 mmol) and the reaction stirred at 25° C. for 16 h. The reaction was quenched H₂O (10 mL) and extracted with DCM (20 mL×3). The combined organic phase was washed with brine (10 mL), dried over Na₂SO₄, filtered and concentrated in vacuo. The crude was purified by column chromatography (PE/EtOAc=5/1 to 1/1) on silica gel to give 4-(3-(benzyloxy)cyclobutoxy)-2-chloro-6-(1,1-difluoroethyl)pyridine (300 mg, 46.9% yield) as yellow gum. LCMS m/z=353.9 [M+H]⁺

### Preparation 111: 3-((2-chloro-6-(1,1-difluoroethyl)pyridin-4-yl)oxy)cyclobutan-1-ol

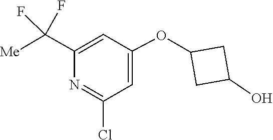 [SMILES: CC(F)(F)c1cc(OC2CC(O)C2)cc(Cl)n1]

A solution of 4-(3-(benzyloxy)cyclobutoxy)-2-chloro-6-(1,1-difluoroethyl)pyridine (Preparation 110, 220.0 mg, 0.622 mmol) in TFA (10 mL) was stirred at 100° C. for 16 h. The cooled reaction was treated with H₂O (20 mL) and extracted with EtOAc (15 mL×3). The combined organic phase was washed with brine (30 mL), dried over Na₂SO₄, filtered and concentrated in vacuo. The residue was purified by column chromatography (PE/EtOAc=5/1 to 3/1) on silica gel to give 3-((2-chloro-6-(1,1-difluoroethyl)pyridin-4-yl)oxy)cyclobutan-1-ol (80.0 mg, 48.8% yield) as yellow solid. LCMS m/z=359.6 [M+H]⁺

### Preparation 112: 2-chloro-6-(1,1-difluoroethyl)-4-(3-methoxycyclobutoxy)pyridine

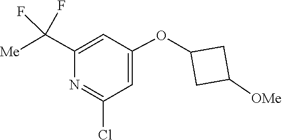 [SMILES: COC1CC(Oc2cc(Cl)nc(C(C)(F)F)c2)C1]

2-Chloro-6-(1,1-difluoroethyl)-4-(3-methoxycyclobutoxy)pyridine was obtained as a colorless oil, 20 mg, 27.1% yield, from 3-((2-chloro-6-(1,1-difluoroethyl)pyridin-4-yl)oxy)cyclobutan-1-ol (Preparation 111) and CH₃I, following a similar procedure to that described in Preparation 107. LCMS m/z=277.6 [M+H]⁺

### Preparation 113: 2-chloro-4-((1-methoxypropan-2-yl)oxy)-6-(3-methoxytetrahydro furan-3-yl)pyridine

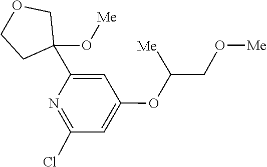 [SMILES: COCC(C)Oc1cc(Cl)nc(C2(OC)CCOC2)c1]

To a solution of 2-chloro-6-(3-methoxytetrahydrofuran-3-yl)pyridin-4-ol (Preparation 90, 500 mg, 2.18 mmol), 1-methoxypropan-2-ol (392.4 mg, 4.35 mmol) and PPh₃ (1.1 g, 4.35 mmol) in THF (10 mL) was added DIAD (880.5 mg, 4.35 mmol) under N₂ at 0° C. and the reaction was stirred at 20° C. for 2 h. The mixture was concentrated under reduced pressure and the residue was purified on silica gel column chromatography (PE/EtOAc=3/1) to give 2-chloro-4-((1-methoxypropan-2-yl)oxy)-6-(3-methoxytetrahydrofuran-3-yl)pyridine (485 mg, 73.8% yield) as a colorless oil. 1H NMR (500 MHz, CDCl₃) δ ppm: 7.01 (s, 1H), 6.76 (s, 1H), 4.66-4.63 (m, 1H), 4.12-4.06 (m, 3H), 3.95-3.93 (m, 1H), 3.95-3.93 (m, 1H), 3.56-3.51 (m, 1H), 3.39 (s, 3H), 3.20 (s, 3H), 2.63-2.60 (m, 1H), 2.31-2.28 (m, 1H), 1.33 (d, J=6.0 Hz, 3H).

### Preparation 114: 2-chloro-4-(2-methoxypropoxy)-6-(3-methoxytetrahydrofuran-3-yl)pyridine

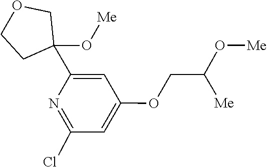 [SMILES: COC(C)COc1cc(Cl)nc(C2(OC)CCOC2)c1]

2-Chloro-4-(2-methoxypropoxy)-6-(3-methoxytetrahydrofuran-3-yl)pyridine was obtained as a colorless oil (520 mg, 79%) from 2-chloro-6-(3-methoxytetrahydrofuran-3-yl)pyridin-4-ol (Preparation 90) and 2-methoxypropanan-1-ol, following the method described in Preparation 113. 1H NMR (400 MHz, CDCl₃) δ ppm: 7.01 (s, 1H), 6.76 (s, 1H), 4.98-4.95 (m, 2H), 4.11-4.00 (m, 4H), 3.73-3.72 (m, 1H), 3.43 (s, 3H), 3.20 (s, 3H), 2.64-2.60 (m, 1H), 2.32-2.31 (m, 1H), 1.23 (s, 3H).

### Preparation 115: 2-chloro-4-(2-methoxyethoxy)-6-(3-methoxytetrahydrofuran-3-yl)pyridine

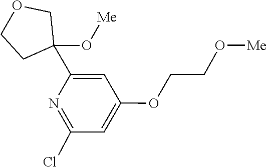 [SMILES: COCCOc1cc(Cl)nc(C2(OC)CCOC2)c1]

2-Chloro-4-(2-methoxyethoxy)-6-(3-methoxytetrahydrofuran-3-yl)pyridine was obtained as a white solid, 175 mg, 46.6% yield, from 2-chloro-6-(3-methoxytetrahydrofuran-3-yl)pyridin-4-ol (Preparation 90) and 2-methoxyethan-1-ol, following a similar procedure to that described in Preparation 113. ¹H NMR: (400 MHz, CDCl₃) δ: ppm 7.06-7.03 (m, 1H), 6.80-6.77 (m, 1H), 4.19-4.06 (m, 5H), 3.96-3.95 (m, 1H), 3.78-3.75 (m, 2H), 3.45 (s, 3H), 3.20 (s, 3H), 2.64-2.60 (m, 1H), 2.32-2.29 (m, 1H).

### Preparation 116: 1-(6-chloro-4-(2-methoxyethoxy)pyridin-2-yl)ethan-1-one

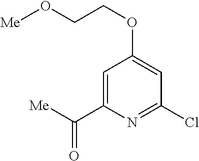 [SMILES: COCCOc1cc(Cl)nc(C(C)=O)c1]

To a solution of 2-chloro-4-(2-methoxyethoxy)pyridine (2.6 g, 13.9 mmol) in MeCN (30 mL) was added 2-hydroperoxy-2-methylpropane, acetaldehyde (5 M, 27.7 mL), TFA (1.7 g, 15.24 mmol) and FeSO₄ (3.9 g, 13.9 mmol) and the reaction was stirred at 80° C. for 16 h. The reaction was concentrated in vacuo and poured into H₂O (60 mL) and extracted with EtOAc (3×80 mL), the combined organic layer was dried over anhydrous Na₂SO₄, filtered and concentrated in vacuo. The residue was purified by chromatography on silica gel (PE/EtOAc=3/1) and the product further purified by Prep-HPLC-K (gradient: 33-63% MeCN) to give 1-(6-chloro-4-(2-methoxyethoxy)pyridin-2-yl)ethan-1-one (42 mg, 1.3% yield) as a yellow solid. ¹H NMR: (500 MHz, CDCl₃) δ ppm: 7.50 (d, J=2.0 Hz, 1H), 7.03 (d, J=2.0 Hz, 1H), 4.23-4.21 (m, 2H), 3.77-3.75 (m, 2H), 3.44 (s, 3H), 2.68 (s, 3H).

### Preparation 117: 2-chloro-6-(1,1-difluoroethyl)-4-(2-methoxyethoxy)pyridine

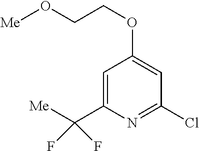 [SMILES: COCCOc1cc(Cl)nc(C(C)(F)F)c1]

To a solution of 1-(6-chloro-4-(2-methoxyethoxy)pyridin-2-yl)ethan-1-one (Preparation 116, 42.0 mg, 0.183 mmol) in DCM (2 mL) was added DAST (58.9 mg, 0.366 mmol) at 0° C., then the resulting mixture was stirred at 50° C. for 24 h. The mixture was quenched with Na₂CO₃ (aq. 5 mL), poured into H₂O (10 mL) and extracted with EtOAc (2×10 mL). The combined organic phase was washed with brine (2×10 mL), dried over Na₂SO₄, filtered and concentrated under reduced pressure. The residue was purified by prep-TLC (PE/EtOAc=3/1) to give 2-chloro-6-(1,1-difluoroethyl)-4-(2-methoxyethoxy)pyridine (20 mg, 43.5% yield) as yellow oil. 1H NMR (500 MHz, CDCl₃) δ ppm: 7.13 (d, J=1.5 Hz, 1H), 6.90 (s, 1H), 4.21-4.19 (m, 2H), 3.77-3.75 (m, 2H), 3.44 (s, 3H), 1.97 (t, J=18.0 Hz, 3H).

### Preparation 118: 2-fluoro-6-(furan-3-yl)pyridine

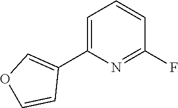 [SMILES: Fc1cccc(-c2ccoc2)n1]

To a solution of 2-bromo-6-fluoropyridine (300 mg, 1.70 mmol) in dioxane (4 mL) and H₂O (0.5 mL) was added furan-3-ylboronic acid (209.8 mg, 1.88 mmol), Pd(dppf)Cl₂ (124.7 mg, 0.170 mmol) and Cs₂CO₃ (1.1 g, 3.41 mmol) and the reaction was stirred at 90° C. for 3 h under N₂. The mixture was concentrated under reduced pressure and the residue was purified by chromatography on silica gel (PE/EtOAc=10/1) to give 2-fluoro-6-(furan-3-yl)pyridine (215.9 mg, 77.6% yield) as colorless oil. ¹H NMR: (500 MHz, CDCl₃) δ ppm: 8.04 (s, 1H), 7.77-7.74 (m, 1H), 7.49-7.48 (m, 1H), 7.33-7.31 (m, 1H), 6.86 (s, 1H), 6.78-6.77 (m, 1H).

### Preparation 119: 2-fluoro-6-(tetrahydrofuran-3-yl)pyridine

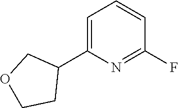 [SMILES: Fc1cccc(C2CCOC2)n1]

To a solution of 2-fluoro-6-(furan-3-yl)pyridine (Preparation 118, 195 mg, 1.20 mmol) in MeOH (5.0 mL) was added Pd/C (254.4 mg, 0.239 mmol, 10% purity) and the mixture was stirred at 25° C. for 4 h under 15 psi of H₂. The reaction was filtered and concentrated in vacuo to give 2-fluoro-6-(tetrahydrofuran-3-yl)pyridine (171.3 mg, 85.7% yield) as colorless oil. ¹H NMR: (400 MHz, CDCl₃) δ ppm: 7.74-7.67 (m, 1H), 7.10-7.07 (m, 1H), 6.78-6.75 (m, 1H), 4.17-4.04 (m, 2H), 3.94-3.86 (m, 2H), 3.55-3.51 (m, 1H), 2.34-2.32 (m, 1H), 2.24-2.21 (m, 1H).

### Preparation 120: 2-fluoro-6-(furan-3-yl)-4-methylpyridine

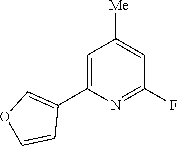 [SMILES: Cc1cc(F)nc(-c2ccoc2)c1]

2-Fluoro-6-(furan-3-yl)-4-methylpyridine was obtained as a colorless oil, 226 mg, 80.8% yield, from 2-bromo-6-fluoro-4-methylpyridine and furan-3-ylboronic acid, following the procedure described in Preparation 118. ¹H NMR: (400 MHz, CDCl₃) δ ppm: 8.02 (s, 1H), 7.48-7.46 (m, 1H), 7.14 (s, 1H), 6.84 (d, J=1.2 Hz, 1H), 6.59 (s, 1H), 2.40 (s, 3H).

### Preparation 121: 2-fluoro-4-methyl-6-(tetrahydrofuran-3-yl)pyridine

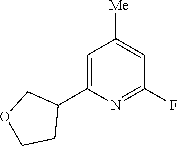 [SMILES: Cc1cc(F)nc(C2CCOC2)c1]

2-Fluoro-4-methyl-6-(tetrahydrofuran-3-yl)pyridine was obtained as a colorless oil, 197 mg, 85.4% yield, from 2-fluoro-6-(furan-3-yl)-4-methylpyridine (Preparation 120) following a similar procedure to that described in Preparation 119. ¹H NMR: (400 MHz, CDCl₃) δ ppm: 6.90 (s, 1H), 6.58 (s, 1H), 4.16-4.11 (m, 1H), 4.06-4.04 (m, 1H), 3.94-3.82 (m, 2H), 3.49-3.47 (m, 1H), 2.36 (s, 3H), 2.33-2.30 (m, 1H), 2.23-2.19 (m, 1H).

### Preparation 122: 2-bromo-6-(furan-3-yl)-4-methoxypyridine

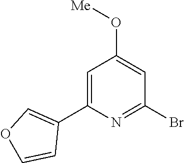 [SMILES: COc1cc(Br)nc(-c2ccoc2)c1]

2-Bromo-6-(furan-3-yl)-4-methoxypyridine was obtained as a white solid, 300 mg, 63% yield, from 2,6-dibromo-4-methoxypyridine and furan-3-ylboronic acid, following the procedure described in Preparation 118. LCMS m/z=254.1 [M+H]⁺

### Preparation 123: 3-(6-bromo-4-methylpyridin-2-yl)tetrahydrofuran-3-ol

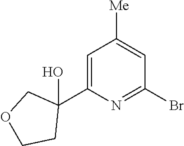 [SMILES: Cc1cc(Br)nc(C2(O)CCOC2)c1]

To a solution of 2,6-dibromo-4-methylpyridine (1.0 g, 3.99 mmol) in DCM (10 mL) was added n-BuLi (2.5 M, 1.59 mL) dropwise at −78° C. over 30 mins, then a solution of dihydrofuran-3(2H)-one (343.1 mg, 3.99 mmol) in DCM (5 mL) was added slowly to the mixture at −78° C. The reaction was allowed to warm up to 15° C. for 2 h under N₂. The mixture was concentrated under vacuum and the crude product was purified on silica gel column chromatography (PE/EtOAc=10/1 to 5/1) to give 3-(6-bromo-4-methylpyridin-2-yl)tetrahydrofuran-3-ol (570 mg, 55.4% yield) as a yellow solid. LCMS m/z=258.0 [M+H]⁺

### Preparation 124: 2-bromo-6-(3-methoxytetrahydrofuran-3-yl)-4-methylpyridine

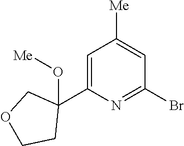 [SMILES: COC1(c2cc(C)cc(Br)n2)CCOC1]

2-Bromo-6-(3-methoxytetrahydrofuran-3-yl)-4-methylpyridine was obtained as a brown oil, 500 mg, 94.9% yield, from 3-(6-bromo-4-methylpyridin-2-yl)tetrahydrofuran-3-ol (Preparation 123) and CH₃I, following a similar procedure to that described in Preparation 54. LCMS m/z=272.1 [M+H]⁺

### Preparation 125: 2,6-dichloro-4-(2-methoxypropoxy)pyridine

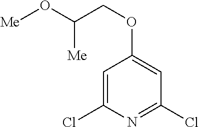 [SMILES: COC(C)COc1cc(Cl)nc(Cl)c1]

2,6-Dichloro-4-(2-methoxypropoxy)pyridine was obtained as a yellow oil, 690 mg, 95.9% yield, from 2,6-dichloropyridin-4-ol and 2-methoxypropan-1-ol following a similar procedure to that described in Preparation 113. ¹H NMR: (500 MHz, CDCl₃) δ ppm: 6.81 (s, 2H), 4.00-3.94 (m, 2H), 3.72-3.70 (m, 1H), 3.42 (s, 3H), 1.25 (d, J=6.5 Hz, 3H).

### Preparation 126: 2-chloro-6-(furan-3-yl)-4-(2-methoxypropoxy)pyridine

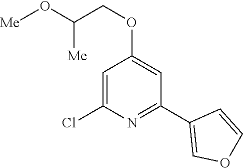 [SMILES: COC(C)COc1cc(Cl)nc(-c2ccoc2)c1]

To a solution of 2,6-dichloro-4-(2-methoxypropoxy)pyridine (Preparation 125, 680 mg, 2.88 mmol) in dioxane (10 mL) and H₂O (1 mL) was added furan-3-ylboronic acid (257.8 mg, 2.30 mmol), K₂CO₃ (796.1 mg, 5.76 mmol) and Pd(dppf)Cl₂ (210.8 mg, 0.288 mmol) and the reaction was stirred at 100° C. for 3 h under N₂. The mixture was concentrated under vacuum and the crude product was purified by silica gel column chromatography (PE/EtOAc=1/0 to 20/1) to give 2-chloro-6-(furan-3-yl)-4-(2-methoxypropoxy)pyridine (410 mg, 53.2% yield) as yellow oil. LCMS m/z=268.1 [M+H]⁺

### Preparation 127: 2,4-dichloro-6-(1,1-difluoroethyl)pyridine

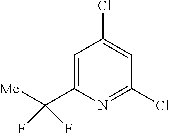 [SMILES: CC(F)(F)c1cc(Cl)cc(Cl)n1]

2,4-Dichloro-6-(1,1-difluoroethyl)pyridine was obtained, 2.0 g, 75.8% yield, from 1-(4,6-dichloropyridin-2-yl)ethanone and DAST following a similar procedure to that described in Preparation 117. 1H NMR: (400 MHz, CDCl₃) δ ppm: 7.59 (s, 1H), 7.43 (s, 1H), 2.00 (t, J=18.8 Hz, 3H).

### Preparation 128: 2-chloro-4-(2-fluoropropan-2-yl)pyridine

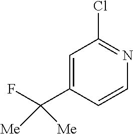 [SMILES: CC(C)(F)c1ccnc(Cl)c1]

To a solution of 1-(2-chloropyridin-4-yl)ethan-1-one (4.8 g, 30.85 mmol) in DCM (50 mL) was added DAST (14.9 g, 92.56 mmol) and the resulting mixture was stirred at 25° C. for 16 h. The mixture was quenched with H₂O (30 mL) and extracted with DCM (30 mL×2). The combined organic phase was washed with brine (30 mL×2), dried over Na₂SO₄ and filtered. The filtrate was concentrated under reduced pressure and the residue was purified by chromatography on silica gel (PE/EtOAc=5/1) to give 2-chloro-4-(2-fluoropropan-2-yl)pyridine (4.8 g, 87.6% yield) as a yellow oil. ¹H NMR: (500 MHz, CDCl₃) δ ppm: 8.51-8.49 (m, 1H), 7.45 (s, 1H), 7.34-7.32 (m, 1H), 1.96-1.87 (m, 3H).

### Preparation 129: 2-chloro-6-(1,1-difluoroethyl)-4-isopropoxypyridine

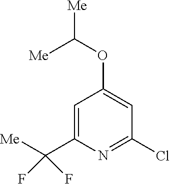 [SMILES: CC(C)Oc1cc(Cl)nc(C(C)(F)F)c1]

A solution of Na (21.7 mg, 0.943 mmol) in i-PrOH (5 mL) was stirred at 50° C. for 2 h, then 2,4-dichloro-6-(1,1-difluoroethyl)pyridine (Preparation 127, 200 mg, 0.943 mmol) was added. The mixture was stirred at 60° C. for 18 h, then concentrated in vacuo. The residue was purified by chromatography on silica gel (PE/EtOAc=5/1) to give 2-chloro-6-(1,1-difluoroethyl)-4-isopropoxypyridine (62 mg, 27.9% yield) as a yellow oil. 1H NMR: (500 MHz, CDCl₃) δ ppm: 7.05 (s, 1H), 6.82 (s, 1H), 4.68-4.63 (m, 1H), 2.01-1.94 (m, 3H), 1.38 (d, J=6.0 Hz, 6H).

### Preparation 130: 2-(6-bromopyridin-2-yl)propan-2-ol

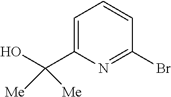 [SMILES: CC(C)(O)c1cccc(Br)n1]

To a solution of 1-(6-bromopyridin-2-yl)ethan-1-one (1 g, 5.0 mmol) in THF (10 mL) was added CH₃MgBr (3 M, 2.50 mL) slowly at 0° C. and the reaction stirred for 12 h under N₂. The reaction was concentrated, diluted with H₂O (50 mL) and extracted with EtOAc (20 mL×3). The combined organic phase was washed with brine (20 mL×2), dried over Na₂SO₄ and filtered. The mixture was concentrated and the residue was purified by chromatography on silica gel (PE/EtOAc=3/1) to give 2-(6-bromopyridin-2-yl)propan-2-ol (910 mg, 84.% yield) as a white oil. ¹H NMR: (400 MHz, CDCl₃) δ ppm: 7.57-7.53 (m, 1H), 7.39-7.26 (m, 2H), 4.06 (br s, 1H), 1.54 (s, 6H).

### Preparation 131: 2-bromo-6-(2-methoxypropan-2-yl)pyridine

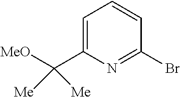 [SMILES: COC(C)(C)c1cccc(Br)n1]

2-Bromo-6-(2-methoxypropan-2-yl)pyridine was obtained as a yellow gum, 432 mg, 81.1% yield, from 2-(6-bromopyridin-2-yl)propan-2-ol (Preparation 130) and CH₃I, following the procedure described in Preparation 54. 1H NMR: (400 MHz, CDCl₃) δ ppm: 7.55-7.49 (m, 2H), 7.35-7.33 (m, 1H), 3.18 (s, 3H), 1.53 (s, 6H).

### Preparation 132: 2-bromo-6-(2-fluoropropan-2-yl)pyridine

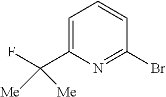 [SMILES: CC(C)(F)c1cccc(Br)n1]

To a solution of 2-(6-bromopyridin-2-yl)propan-2-ol (Preparation 130, 200 mg, 0.926 mmol) in DCM (3.0 mL) was added DAST (298.4 mg, 1.85 mmol) and the reaction was stirred at 20° C. for 12 h. The mixture was quenched with aq. Na₂CO₃ (20 mL) and extracted with DCM (20 mL×2). The combined organic phase was washed with brine (20 mL×2), dried over Na₂SO₄ and filtered. The filtrate was concentrated under reduced pressure and the residue was purified by chromatography on silica gel (PE/EtOAc=3/1) to give 2-bromo-6-(2-fluoropropan-2-yl)pyridine (190 mg, 94.1% yield) as a yellow oil. ¹H NMR: (400 MHz, CDCl₃) δ ppm: 7.58-7.49 (m, 2H), 7.38 (d, J=7.6 Hz, 1H), 1.73 (s, 3H), 1.67 (s, 3H).

### Preparation 133: 2-chloro-6-(1,1-difluoroethyl)-4-methoxypyridine

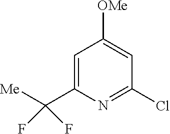 [SMILES: COc1cc(Cl)nc(C(C)(F)F)c1]

To a solution of 2,4-dichloro-6-(1,1-difluoroethyl)pyridine (Preparation 127, 151 mg, 0.726 mmol) in MeOH (6 mL) was added MeONa (47.1 mg, 0.871 mmol) and the reaction was stirred at 30° C. for 36 h. The mixture was concentrated under reduced pressure and the residue was purified by chromatography on silica gel (PE/EtOAc=5/1) to give 2-chloro-6-(1,1-difluoroethyl)-4-methoxypyridine (40 mg, 27.1% yield) as a yellow oil. 1H NMR: (400 MHz, CDCl₃) δ ppm: 7.01 (s, 1H), 6.72 (d, J=1.6 Hz, 1H), 3.87 (s, 3H), 1.70 (s, 3H), 1.65 (s, 3H).

Alternative synthesis: To a solution of 1-(6-chloro-4-methoxypyridin-2-yl)ethan-1-one (Preparation 134, 328 mg, 1.77 mmol) in DCM (5 mL) was added DAST (3.7 g, 3.0 mL) and the reaction was stirred at 25° C. for 48 h. The mixture was concentrated in vacuo to dryness and the crude was purified by column chromatography (PE) to give 2-chloro-6-(1,1-difluoroethyl)-4-methoxypyridine (87.0 mg, 23.7% yield) as colorless oil. ¹H NMR: (500 MHz, CDCl₃) δ ppm: 7.11 (d, J=2.0 Hz, 1H), 6.88 (d, J=2.0 Hz, 1H), 3.91 (s, 3H), 2.03-1.95 (m, 3H).

### Preparation 134: 1-(6-chloro-4-methoxypyridin-2-yl)ethan-1-one

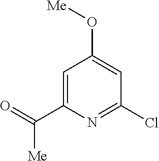 [SMILES: COc1cc(Cl)nc(C(C)=O)c1]

To a solution of 2-chloro-4-methoxypyridine (1.0 g, 6.97 mmol), 2-oxopropanoic acid (613.4 mg, 6.97 mmol) and AgNO₃ (236.6 mg, 1.39 mmol) in DCM (5 mL) and H₂O (5 mL) was added K₂S₂O₈ (3.8 g, 13.9 mmol) and the reaction was stirred at 25° C. for 16 h. The mixture was diluted with water (15 mL) and extracted with EtOAc (20 mL×3). The combined organic phase was washed with brine (60 mL), dried over anhydrous Na₂SO₄, filtered and concentrated in vacuo. The crude was purified by column chromatography (PE/EtOAc=1/0 to 8/1) to give 1-(6-chloro-4-methoxypyridin-2-yl)ethan-1-one (86.0 mg, 6.7% yield) as a white solid. LCMS m/z=186.1 [M+H]⁺

### Preparation 135: 2-bromo-6-(oxetan-3-yl)pyridine

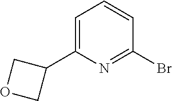 [SMILES: Brc1cccc(C2COC2)n1]

To a solution of 2,6-dibromopyridine (1 g, 4.22 mmol) and 3-bromoxetane (1.3 g, 8.44 mmol) in DME (20 mL) were added dtbbpy (113.1 mg, 0.422 mmol), Ir[dF(CF₃)ppy]₂(dtbbpy)PF₆ (47.4 mg, 0.042 mmol), LiOH (202.2 mg, 8.44 mmol), NiCl₂·glyme (92.5 mg, 0.422 mmol) and TTMSS (1.1 g, 4.22 mmol) and the mixture was stirred and irradiated with blue LEDs for 12 h at 20° C. under N₂. The reaction was concentrated in vacuo, diluted with H₂O (20 mL) and extracted with EtOAc (3×20 mL). The combined organic phase was washed with brine (2×20 mL), dried over Na₂SO₄, filtered and concentrated under reduced pressure. The residue was purified by chromatography on silica gel (PE/EtOAc=3/1) to give 2-bromo-6-(oxetan-3-yl)pyridine (400 mg, 44.3% yield) as a yellow oil. ¹H NMR: (500 MHz, CDCl₃) δ ppm: 7.54 (t, J=8.0 Hz, 1H), 7.38 (d, J=8.0 Hz, 1H), 7.29 (d, J=7.5 Hz, 1H), 5.05-5.02 (m, 2H), 4.91-4.89 (m, 2H), 4.38-4.35 (m, 1H).

### Preparation 136: 2-chloro-6-(furan-3-yl)-4-(2-methoxyethoxy)pyridine

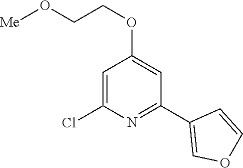 [SMILES: COCCOc1cc(Cl)nc(-c2ccoc2)c1]

2-Chloro-6-(furan-3-yl)-4-(2-methoxyethoxy)pyridine was obtained as a colorless oil, 183 mg, 80.1% yield, from 2,6-dichloro-4-(2-methoxyethoxy)pyridine, following the procedure described in Preparation 126. ¹H NMR: (400 MHz, CDCl₃) δ: ppm 8.04 (d, J=8.0 Hz, 1H), 7.48-7.46 (m, 1H), 6.93-6.88 (m, 1H), 6.81 (d, J=6.0 Hz, 1H), 6.72 (d, J=2.0 Hz, 1H), 4.24-4.15 (m, 2H), 3.78-3.74 (m, 2H), 3.45 (s, 3H).

### Preparation 137: tert-butyl (1-(6-bromopyridin-2-yl)-2,2,2-trifluoroethyl)carbamate

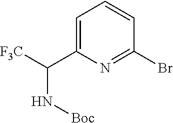 [SMILES: CC(C)(C)OC(=O)NC(c1cccc(Br)n1)C(F)(F)F]

To a solution of 1-(6-bromopyridin-2-yl)-2,2,2-trifluoroethan-1-amine (200 mg, 0.784 mmol) in DCM (5 mL) was added (Boc)₂O (179.7 mg, 0.823 mmol) and TEA (238.1 mg, 2.35 mmol) and the reaction was stirred at 20° C. for 12 h. The reaction was concentrated and purified by column chromatography (PE/EtOAc=10/1 to 5/1) on silica gel to give tert-butyl (1-(6-bromopyridin-2-yl)-2,2,2-trifluoroethyl)carbamate (80.2 mg, 28.8% yield) as a colorless oil. ¹H NMR: (400 MHz, CDCl₃) δ ppm: 7.61-7.57 (m, 1H), 7.52 (d, J=7.6 Hz, 1H), 7.33 (d, J=7.6 Hz, 1H), 6.12-6.10 (m, 1H), 5.42-5.35 (m, 1H), 1.48 (s, 9H).

### Preparation 138: tert-butyl (1-(6-(6-acetamido-1H-pyrazolo[4,3-c]pyridin-1-yl)pyridin-2-yl)-2,2,2-trifluoroethyl)carbamate

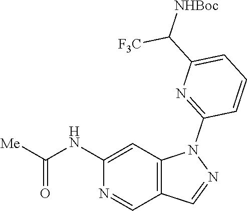 [SMILES: CC(=O)Nc1cc2c(cn1)cnn2-c1cccc(C(NC(=O)OC(C)(C)C)C(F)(F)F)n1]

To a mixture of tert-butyl (1-(6-bromopyridin-2-yl)-2,2,2-trifluoroethyl)carbamate (Preparation 137, 80 mg, 0.225 mmol) and N-(1H-pyrazolo[4,3-c]pyridin-6-yl)acetamide (59.5 mg, 0.338 mmol) in dioxane (3 mL) was added K₂CO₃ (93.4 mg, 0.676 mmol), CuI (8.6 mg, 0.045 mmol) and N, N′-dimethylethane-1, 2-diamine (7.9 mg, 0.090 mmol) and the reaction was stirred at 100° C. under N₂ for 12 h. The mixture was poured into H₂O (15 mL), extracted with EtOAc (10 mL×3), the combined organic layer was dried over anhydrous Na₂SO₄, filtered and concentrated in vacuo. The residue was purified by chromatography (PE/EtOAc=5/1 to 3/1) on silica gel to give tert-butyl (1-(6-(6-acetamido-1H-pyrazolo[4,3-c]pyridin-1-yl)pyridin-2-yl)-2,2,2-trifluoroethyl)carbamate (66.9 mg, 65.9% yield) as colorless oil. 1H NMR: (500 MHz, CDCl₃) δ ppm: 9.66 (s, 1H), 8.79 (s, 1H), 8.23 (s, 1H), 8.13 (s, 1H), 8.05 (d, J=9.0 Hz, 1H), 7.90-7.87 (m, 1H), 6.84 (d, J=9.5 Hz, 1H), 5.60-5.57 (m, 1H), 2.28 (s, 3H), 1.43 (s, 9H).

### Preparation 139: 6-chloro-1-(6-(3-methoxytetrahydrofuran-3-yl)pyridin-2-yl)-1H-pyrazolo[4,3-c]pyridine

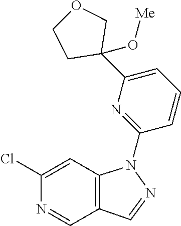 [SMILES: COC1(c2cccc(-n3ncc4cnc(Cl)cc43)n2)CCOC1]

To a solution of 2-bromo-6-(3-methoxytetrahydrofuran-3-yl)pyridine (Preparation 55, 1.0 g, 6.51 mmol) and 6-chloro-1H-pyrazolo[4,3-c]pyridine (1.7 g, 6.51 mmol) in dioxane (40 mL) was added K₂CO₃ (2.7 g, 19.5 mmol), CuI (248.0 mg, 1.30 mmol) and N,N′-dimethylethane-1,2-diamine (229.6 mg, 2.60 mmol) and the reaction was stirred at 100° C. for 6 h. The cooled mixture was treated with H₂O (50 mL), extracted with EtOAc (30 mL×3), the combined organic phase was washed with brine (80 mL), dried over Na₂SO₄, filtered and concentrated. The crude was purified by column chromatography (PE/EtOAc=15/1 to 3/1) on silica gel to give 6-chloro-1-(6-(3-methoxytetrahydrofuran-3-yl)pyridin-2-yl)-1H-pyrazolo[4,3-c]pyridine (1.5 g, 69.6% yield) as a white solid. LCMS m/z=331.1 [M+H]⁺

### Preparation 140: 2-chloro-7-(5-(3-methoxytetrahydrofuran-3-yl)pyridin-3-yl)-7H-pyrrolo[2,3-d]pyrimidine

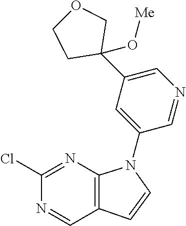 [SMILES: COC1(c2cncc(-n3ccc4cnc(Cl)nc43)c2)CCOC1]

To a solution of 3-bromo-5-(3-methoxytetrahydrofuran-3-yl)pyridine (Preparation 50, 400 mg, 1.55 mmol) and 2-chloro-7H-pyrrolo[2,3-d]pyrimidine (238.0 mg, 1.55 mmol) in dioxane (5 mL) was added N¹,N²-dimethylethane-1,2-diamine (27.3 mg, 0.310 mmol), CuI (118.1 mg, 0.620 mmol) and K₂CO₃ (428.4 mg, 3.10 mmol) and the reaction was stirred at 90° C. for 2 h under N₂. The cooled mixture was concentrated under reduced pressure and the residue was purified by chromatography on silica gel (PE/EtOAc=3/1 to 1/1) to give 2-chloro-7-(5-(3-methoxytetrahydrofuran-3-yl)pyridin-3-yl)-7H-pyrrolo[2,3-d]pyrimidine (260 mg, 50.7% yield) as a yellow oil. ¹H NMR: (400 MHz, CDCl₃) δ ppm: 8.94-8.90 (m, 2H), 8.71-8.68 (m, 1H), 8.25-8.22 (m, 1H), 7.57 (d, J=3.6 Hz, 1H), 6.82 (d, J=3.6 Hz, 1H), 4.11-4.19 (m, 3H), 3.98-3.95 (m, 1H), 3.26 (s, 3H), 2.60-2.56 (m, 1H), 2.40-2.35 (m, 1H).

### Preparation 141: 4-(6-chloro-1-(6-(1,1-difluoroethyl)pyridin-2-yl)-1H-pyrazolo[4,3-c]pyridin-3-yl)morpholine

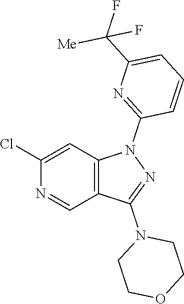 [SMILES: CC(F)(F)c1cccc(-n2nc(N3CCOCC3)c3cnc(Cl)cc32)n1]

4-(6-Chloro-1-(6-(1,1-difluoroethyl)pyridin-2-yl)-1H-pyrazolo[4,3-c]pyridin-3-yl)morpholine was obtained as a white solid (60 mg, 37.7%), from 4-(6-chloro-1H-pyrazolo[4,3-c]pyridin-3-yl)morpholine (Preparation 17) and 2-bromo-6-(1,1-difluoroethyl)pyridine, following the procedure described in Preparation 140. ¹H NMR: (400 MHz, CDCl₃) δ: ppm 8.86 (s, 1H), 8.65 (s, 1H), 7.96-7.90 (m, 2H), 7.47 (d, J=6.8 Hz, 1H), 3.97-3.94 (m, 4H), 3.62-3.59 (m, 4H), 2.12 (t, J=18.4 Hz, 3H).

### Preparation 142: 6-chloro-3-cyclopropyl-1-(6-(1,1-difluoroethyl)pyridin-2-yl)-1H-pyrazolo[4,3-c]pyridine

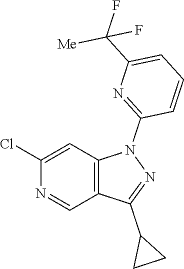 [SMILES: CC(F)(F)c1cccc(-n2nc(C3CC3)c3cnc(Cl)cc32)n1]

6-Chloro-3-cyclopropyl-1-(6-(1,1-difluoroethyl)pyridin-2-yl)-1H-pyrazolo[4,3-c]pyridine was obtained as a white solid, 170 mg, 49.2% from 6-chloro-3-cyclopropyl-1H-pyrazolo[4,3-c]pyridine (Preparation 16) and 2-bromo-6-(1,1-difluoroethyl)pyridine, following the procedure described in Preparation 140. ¹H NMR: (400 MHz, CDCl₃) δ ppm: 8.88 (s, 1H), 8.58 (s, 1H), 8.02 (d, J=8.4 Hz, 1H), 7.94 (t, J=7.6 Hz, 1H), 7.51 (d, J=7.6 Hz, 1H), 2.33-2.29 (m, 1H), 2.12 (t, J=18.4 Hz, 3H), 1.24-1.19 (m, 4H).

### Preparation 143: (3S)-1-(6-chloro-1-(tetrahydro-2H-pyran-2-yl)-1H-pyrazolo[4,3-c]pyridin-3-yl)-N,N-dimethylpyrrolidin-3-amine

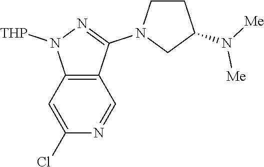

To a solution of 6-chloro-3-iodo-1-(tetrahydro-2H-pyran-2-yl)-1H-pyrazolo[4,3-c]pyridine (500 mg, 1.38 mmol) in DMSO (10 mL) was added (S)—N,N-dimethylpyrrolidin-3-amine (471.1 mg, 4.13 mmol), K₂CO₃ (570.2 mg, 4.13 mmol), CuI (26.2 mg, 0.138 mmol) and L-proline (15.8 mg, 0.138 mmol) and the reaction was stirred at 100° C. for 20 h. The mixture was diluted with water (15 mL) and extracted with EtOAc (20 mL×3). The combined organic phase was washed with brine (60 mL), dried over anhydrous Na₂SO₄, filtered and concentrated. The crude was purified by silica gel column (DCM/MeOH=0/1 to 10/1) to give (3S)-1-(6-chloro-1-(tetrahydro-2H-pyran-2-yl)-1H-pyrazolo[4,3-c]pyridin-3-yl)-N,N-dimethylpyrrolidin-3-amine (261.0 mg, 54.3% yield) as a yellow solid. LCMS m/z=350.1 [M+H]⁺

### Preparation 144: (3R)-1-(6-chloro-1-(tetrahydro-2H-pyran-2-yl)-1H-pyrazolo[4,3-c]pyridin-3-yl)-N,N-dimethylpyrrolidin-3-amine

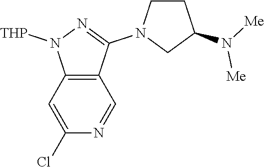

To a solution of 6-chloro-3-iodo-1-(tetrahydro-2H-pyran-2-yl)-1H-pyrazolo[4,3-c]pyridine (500 mg, 1.38 mmol) in DMSO (10 mL) was added (R)—N,N-dimethylpyrrolidin-3-amine (314.1 mg, 2.75 mmol), K₂CO₃ (380.1 mg, 2.75 mmol), L-proline (31.7 mg, 0.275 mmol) and CuI (78.6 mg, 0.413 mmol) and the reaction was stirred at 100° C. for 16 h under N₂. The cooled mixture was quenched with H₂O (10 mL) and extracted with EtOAc (10 mL×2). The combined organic phase was washed with brine (10 mL×2), dried over Na₂SO₄ and filtered. The filtrate was concentrated under reduced pressure and the residue was purified by chromatography on silica gel (PE/EtOAc=0/1) to give (3R)-1-(6-chloro-1-(tetrahydro-2H-pyran-2-yl)-1H-pyrazolo[4,3-c]pyridin-3-yl)-N,N-dimethylpyrrolidin-3-amine (300 mg, 62.4% yield) as a yellow solid. LCMS m/z=350.1 [M+H]⁺

### Preparation 145: (S)-1-(6-chloro-1H-pyrazolo[4,3-c]pyridin-3-yl)-N,N-dimethylpyrrolidin-3-amine

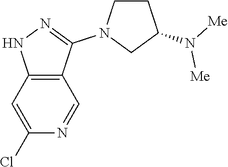 [SMILES: CN(C)[C@H]1CCN(c2n[nH]c3cc(Cl)ncc23)C1]

To a solution of (3S)-1-(6-chloro-1-(tetrahydro-2H-pyran-2-yl)-1H-pyrazolo[4,3-c]pyridin-3-yl)-N,N-dimethylpyrrolidin-3-amine (Preparation 143, 304 mg, 0.869 mmol) in DCM (5 mL) was added TFA (1.3 mL) and the mixture was stirred at 25° C. for 5 h. The mixture was adjusted pH=7 by NaHCO₃ solution, then extracted with EtOAc (20 mL×3). The combined organic phase was washed with brine (10 mL×3), dried over anhydrous Na₂SO₄, filtered and concentrated. The crude product was purified by Prep-HPLC-L (Gradient: 17-47% MeCN) to give (S)-1-(6-chloro-1H-pyrazolo[4,3-c]pyridin-3-yl)-N,N-dimethylpyrrolidin-3-amine (106.2 mg, 46.0% yield) as a white solid. LCMS m/z=266.1 [M+H]⁺

### Preparation 146: (R)-1-(6-chloro-1H-pyrazolo[4,3-c]pyridin-3-yl)-N,N-dimethylpyrrolidin-3-amine

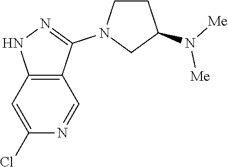 [SMILES: CN(C)[C@@H]1CCN(c2n[nH]c3cc(Cl)ncc23)C1]

A solution of (3R)-1-(6-chloro-1-(tetrahydro-2H-pyran-2-yl)-1H-pyrazolo[4,3-c]pyridin-3-yl)-N,N-dimethylpyrrolidin-3-amine (Preparation 144, 260 mg, 0.743 mmol) in HCl/dioxane (4 M, 5 mL) was stirred at 25° C. for 6 h. The reaction was concentrated to give (R)-1-(6-chloro-1H-pyrazolo[4,3-c]pyridin-3-yl)-N,N-dimethylpyrrolidin-3-amine (210 mg, crude, HCl salt) as a yellow solid. LCMS m/z=266.1 [M+H]⁺

### Preparation 147: (S)-1-(6-chloro-1-(6-(1,1-difluoroethyl)pyridin-2-yl)-1H-pyrazolo[4,3-c]pyridin-3-yl)-N,N-dimethylpyrrolidin-3-amine

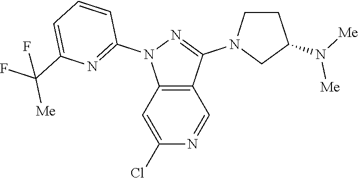 [SMILES: CN(C)[C@H]1CCN(c2nn(-c3cccc(C(C)(F)F)n3)c3cc(Cl)ncc23)C1]

To a solution of (S)-1-(6-chloro-1H-pyrazolo[4,3-c]pyridin-3-yl)-N,N-dimethylpyrrolidin-3-amine (Preparation 145, 100 mg, 0.376 mmol) in dioxane (5 mL) was added 2-bromo-6-(1,1-difluoroethyl)pyridine (100.3 mg, 0.452 mmol), Cs₂CO₃ (245.2 mg, 0.753 mmol) and BrettPhos Pd G₃ (34.1 mg, 0.038 mmol) and the reaction mixture was stirred at 100° C. for 1 h under N₂. The cooled mixture was diluted with water (10 mL) and extracted with EtOAc (15 mL×3). The combined organic phase was washed with brine (30 mL×3), dried over anhydrous Na₂SO₄, filtered and concentrated. The crude was purified by silica gel column (PE/EtOAc=1/1) to give (S)-1-(6-chloro-1-(6-(1,1-difluoroethyl)pyridin-2-yl)-1H-pyrazolo[4,3-c]pyridin-3-yl)-N,N-dimethylpyrrolidin-3-amine (55.3 mg, 36.1% yield) as a yellow solid. LCMS m/z=408.7 [M+H]⁺

### Preparation 148: (R)-1-(6-chloro-1-(6-(1,1-difluoroethyl)pyridin-2-yl)-1H-pyrazolo[4,3-c]pyridin-3-yl)-N,N-dimethylpyrrolidin-3-amine

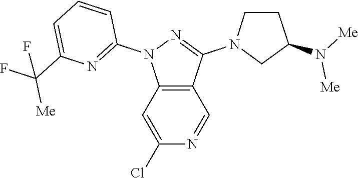 [SMILES: CN(C)[C@@H]1CCN(c2nn(-c3cccc(C(C)(F)F)n3)c3cc(Cl)ncc23)C1]

To a solution of (R)-1-(6-chloro-1H-pyrazolo[4,3-c]pyridin-3-yl)-N,N-dimethylpyrrolidin-3-amine (Preparation 146, 190 mg, 0.715 mmol) in dioxane (5 mL) was added 2-bromo-6-(1,1-difluoroethyl)pyridine (158.8 mg, 0.715 mmol), K₃PO₄ (303.5 mg, 1.43 mmol) and BrettPhos Pd G3 (64.8 mg, 0.0715 mmol) and the reaction was stirred at 100° C. for 16 h under N₂. The mixture was concentrated and was purified by Prep-HPLC-P (Gradient: 63 to 92% MeCN) to give (R)-1-(6-chloro-1-(6-(1,1-difluoroethyl)pyridin-2-yl)-1H-pyrazolo[4,3-c]pyridin-3-yl)-N,N-dimethylpyrrolidin-3-amine (40 mg, 13.8% yield) as a yellow solid. ¹H NMR: (400 MHz, CDCl₃) δ: ppm 8.83 (s, 1H), 8.58 (s, 1H), 7.96-7.87 (m, 2H), 7.42 (d, J=7.2 Hz, 1H), 3.97-3.93 (m, 2H), 3.77-3.75 (m, 1H), 3.56-3.51 (m, 1H), 2.95-2.93 (m, 1H), 2.37 (s, 6H), 2.31-2.29 (m, 1H), 2.16-2.04 (m, 4H).

### Preparation 149: 4-(6-chloro-1-((2-(trimethylsilyl)ethoxy)methyl)-1H-pyrrolo[3,2-c]pyridin-3-yl)morpholine

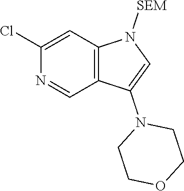 [SMILES: C[Si](C)(C)CCOCn1cc(N2CCOCC2)c2cnc(Cl)cc21]

To a solution of 6-chloro-3-iodo-1-((2-(trimethylsilyl)ethoxy)methyl)-1H-pyrrolo[3,2-c]pyridine (Preparation 28, 300 mg, 0.734 mmol) in DMSO (10 mL) was added K₂CO₃ (304.3 mg, 2.20 mmol), CuI (28.0 mg, 0.147 mmol), L-proline (33.8 mg, 0.294 mmol) and morpholine (383.7 mg, 4.40 mmol) and the reaction was stirred at 100° C. for 1 h under microwave irradiation. The cooled reaction was treated with H₂O (30 mL) and extracted with EtOAc (20 mL×3). The organic phase was washed with brine (30 mL), dried over Na₂SO₄, filtered and concentrated in vacuo. The crude was purified by column chromatography (PE/EtOAc=5/1 to 1/1) on silica gel to give 4-(6-chloro-1-((2-(trimethylsilyl)ethoxy)methyl)-1H-pyrrolo[3,2-c]pyridin-3-yl)morpholine (110 mg, 40.7% yield) as yellow solid. ¹H NMR: (400 MHz, CDCl₃) δ ppm: 8.67 (s, 1H), 7.34 (s, 1H), 6.67 (s, 1H), 5.34 (s, 2H), 3.94-3.91 (m, 4H), 3.48-3.43 (m, 2H), 3.10-3.07 (m, 4H), 0.91-0.87 (m, 2H), −0.04 (s, 9H).

### Preparation 150: 4-(6-chloro-1H-pyrrolo[3,2-c]pyridin-3-yl)morpholine

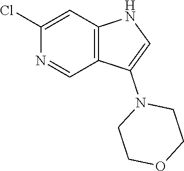 [SMILES: Clc1cc2[nH]cc(N3CCOCC3)c2cn1]

To a solution of 4-(6-chloro-1-((2-(trimethylsilyl)ethoxy)methyl)-1H-pyrrolo[3,2-c]pyridin-3-yl)morpholine (Preparation 149, 110 mg, 0.299 mmol) in DCM (5 mL) was added TFA (1 M, 1.0 mL) and the reaction stirred at 25° C. for 16 h. NH₃·H₂O (10.48 mg, 0.299 mmol) was added slowly and the mixture was stirred at 25° C. for 16 h. The reaction was treated with H₂O (10 mL), extracted with DCM (15 mL×3), the combined organic phase was washed with brine (20 mL), dried over Na₂SO₄, filtered and concentrated in vacuo. The crude was purified by column chromatography (PE/EtOAc=3/1 to 0/1) on silica gel to give 4-(6-chloro-1H-pyrrolo[3,2-c]pyridin-3-yl)morpholine (60.0 mg, 84.4% yield) as yellow solid. LCMS m/z=238.1 [M+H]⁺

### Preparation 151: 4-(6-chloro-1-(4-(1,1-difluoroethyl)pyridin-2-yl)-1H-pyrrolo[3,2-c]pyridin-3-yl)morpholine

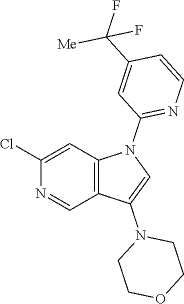 [SMILES: CC(F)(F)c1ccnc(-n2cc(N3CCOCC3)c3cnc(Cl)cc32)c1]

To a solution of 4-(6-chloro-1H-pyrrolo[3,2-c]pyridin-3-yl)morpholine (Preparation 150, 45.0 mg, 0.189 mmol) and 2-chloro-4-(1,1-difluoroethyl)pyridine (33.6 mg, 0.189 mmol) in toluene (5.0 mL) was added Cs₂CO₃ (185.1 mg, 0.568 mmol) and BrettPhos Pd G₃ (17.2 mg, 0.019 mmol) and the reaction stirred at 100° C. for 1 h under N₂. The cooled mixture was concentrated under reduced pressure and the residue was purified by column chromatography (PE/EtOAc=15/1 to 5/1) on silica gel to give 4-(6-chloro-1-(4-(1,1-difluoroethyl)pyridin-2-yl)-1H-pyrrolo[3,2-c]pyridin-3-yl)morpholine (32.0 mg, 44.6% yield) as light yellow solid. LCMS m/z=379.1 [M+H]⁺

### Preparation 152: 1-(6-(3-methoxytetrahydrofuran-3-yl)pyridin-2-yl)-1H-pyrazolo[4,3-c]pyridin-6-amine

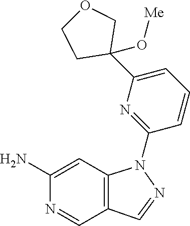 [SMILES: COC1(c2cccc(-n3ncc4cnc(N)cc43)n2)CCOC1]

To a solution of 6-chloro-1-(6-(3-methoxytetrahydrofuran-3-yl)pyridin-2-yl)-1H-pyrazolo[4,3-c]pyridine (Preparation 139, 500 mg, 1.51 mmol) and NH₂Boc (159.4 mg, 1.36 mmol) in dioxane (10 mL) was added BINAP (47.1 mg, 0.076 mmol), Pd(OAc)₂ (13.6 mg, 0.060 mmol) and Cs₂CO₃ (689.5 mg, 2.12 mmol) and the reaction was stirred at 100° C. for 16 h. The cooled mixture was treated with H₂O (15 mL) and extracted with EtOAc (20 mL×3). The combined organic phase was washed with brine (20 mL), dried over Na₂SO₄, filtered, concentrated, then purified by column chromatography (PE/EtOAc=15/1 to 5/1) on silica gel to give 1-(6-(3-methoxytetrahydrofuran-3-yl)pyridin-2-yl)-1H-pyrazolo[4,3-c]pyridin-6-amine (180 mg, 38.3% yield) as yellow solid. LCMS m/z=312.2 [M+H]⁺

### Preparation 153: tert-butyl (7-(5-(3-methoxytetrahydrofuran-3-yl)pyridin-3-yl)-7H-pyrrolo[2,3-d]pyrimidin-2-yl)carbamate

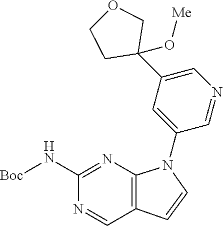 [SMILES: COC1(c2cncc(-n3ccc4cnc(NC(=O)OC(C)(C)C)nc43)c2)CCOC1]

A mixture of 2-chloro-7-(5-(3-methoxytetrahydrofuran-3-yl)pyridin-3-yl)-7H-pyrrolo[2,3-d]pyrimidine (Preparation 140, 250 mg, 0.756 mmol), NH₂Boc (88.5 mg, 0.755 mmol), BINAP (94.1 mg, 0.151 mmol), Pd(OAc)₂ (17.0 mg, 0.076 mmol) and Cs₂CO₃ (492.5 mg, 1.51 mmol) in dioxane (5 mL) was stirred at 110° C. for 2 h under N₂. The cooled reaction was concentrated under reduced pressure and the residue was purified by chromatography on silica gel (PE/EtOAc=1/1 to 1/2) to give tert-butyl (7-(5-(3-methoxytetrahydrofuran-3-yl)pyridin-3-yl)-7H-pyrrolo[2,3-d]pyrimidin-2-yl)carbamate (205 mg, 65.9% yield) as a yellow solid. ¹H NMR: (400 MHz, CDCl₃) δ ppm: 8.93 (d, J=2.4 Hz, 1H), 8.87-8.85 (m, 2H), 8.68 (s, 1H), 7.55 (d, J=4.0 Hz, 1H), 6.77 (d, J=3.6 Hz, 1H), 4.19-4.11 (m, 3H), 4.04-4.02 (m, 1H), 3.24 (s, 3H), 2.62-2.52 (m, 2H), 1.54 (s, 9H).

### Preparation 154: 7-(5-(3-methoxytetrahydrofuran-3-yl)pyridin-3-yl)-7H-pyrrolo[2,3-d]pyrimidin-2-amine

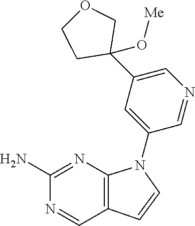 [SMILES: COC1(c2cncc(-n3ccc4cnc(N)nc43)c2)CCOC1]

A mixture of tert-butyl (7-(5-(3-methoxytetrahydrofuran-3-yl)pyridin-3-yl)-7H-pyrrolo[2,3-d]pyrimidin-2-yl)carbamate (Preparation 153, 205 mg, 0.498 mmol) in HCl/dioxane (4 M, 5 mL) was stirred at 25° C. for 1 h. The reaction was concentrated under reduced pressure to give 7-(5-(3-methoxytetrahydrofuran-3-yl)pyridin-3-yl)-7H-pyrrolo[2,3-d]pyrimidin-2-amine hydrochloride (162 mg, 93.5% yield) as a white solid. LCMS m/z=312.2 [M+H]⁺

### Preparation 155: N-(3-(4-methylpiperazin-1-yl)-1-(tetrahydro-2H-pyran-2-yl)-1H-pyrazolo[4,3-c]pyridin-6-yl)acetamide

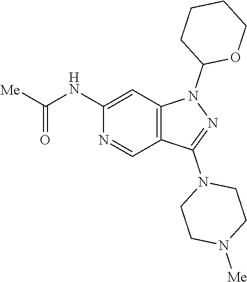 [SMILES: CC(=O)Nc1cc2c(cn1)c(N1CCN(C)CC1)nn2C1CCCCO1]

A mixture of N-(3-iodo-1-(tetrahydro-2H-pyran-2-yl)-1H-pyrazolo[4,3-c]pyridin-6-yl)acetamide (Preparation 12, 2 g, 5.18 mmol), 1-methylpiperazine (1.6 g, 15.54 mmol), L-proline (119.3 mg, 1.04 mmol), K₂CO₃ (1.4 g, 10.4 mmol) and CuI (296 mg, 1.55 mmol) in DMSO (20 mL) was stirred at 100° C. under N₂ for 16 h. The mixture was evaporated to dryness and the residue purified by prep-HPLC-A (Gradient=13-43% MeCN) to give N-(3-(4-methylpiperazin-1-yl)-1-(tetrahydro-2H-pyran-2-yl)-1H-pyrazolo[4,3-c]pyridin-6-yl)acetamide as a yellow solid (1.2 g, 65%). LCMS m/z=359.2 [M+H]⁺.

### Preparations 156 to 158

The title compounds were prepared from N-(3-iodo-1-(tetrahydro-2H-pyran-2-yl)-1H-pyrazolo[4,3-c]pyridin-6-yl)acetamide (Preparation 12) and the appropriate amine using an analogous method to that described for Preparation 155.

| Preparation | |
|---|---|
| No | Name/Structure/Amine/Data |
| 156 | N-(3-(3-cyanoazetidin-1-yl)-1-(tetrahydro-2H-pyran-2-yl)-1H-pyrazolo[4,3- |
| | c]pyridin-6-yl)acetamide |
| | 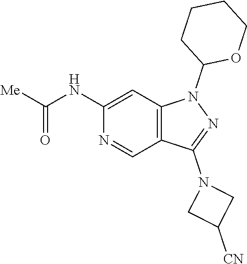 [SMILES: CC(=O)Nc1cc2c(cn1)c(N1CC(C#N)C1)nn2C1CCCCO1] |
| | Amine: 3-cycanoazetidine; Yellow oil (120 mg, 45%); LCMS m/z = 341.0 |
| | [M + H]⁺. |
| 157 | N-(3-(pyrrolidin-1-yl)-1-(tetrahydro-2H-pyran-2-yl)-1H-pyrazolo[4,3- |
| | c]pyridin-6-yl)acetamide |
| | 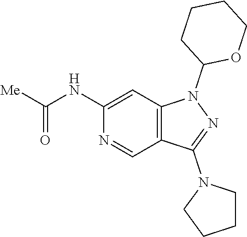 [SMILES: CC(=O)Nc1cc2c(cn1)c(N1CCCC1)nn2C1CCCCO1] |
| | Amine: pyrrolidine; Yellow oil (130 mg, 38%); LCMS m/z = 330.2 |
| | [M + H]⁺. |
| 158 | N-(3-(4-methoxypiperidin-1-yl)-1-(tetrahydro-2H-pyran-2-yl)-1H- |
| | pyrazolo[4,3-c]pyridin-6-yl)acetamide |
| | 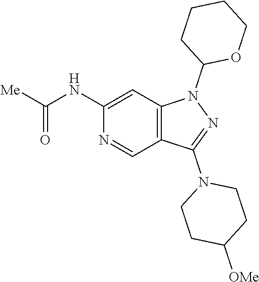 [SMILES: COC1CCN(c2nn(C3CCCCO3)c3cc(NC(C)=O)ncc23)CC1] |
| | Amine: 4-methoxypiperidine; Yellow oil (180 mg, 47%); ¹H NMR (400 |
| | MHz, CDCl₃) δ: 8.65 (s, 1H), 8.53 (br s, 1H), 8.19 (s, 1H), 5.49-5.47 (m, |
| | 1H), 4.14-4.10 (m, 2H), 4.07-3.71 (m, 4H), 3.38 (s, 3H), 3.20-3.18 (m, 2H), |
| | 2.50-2.48 (m, 2H), 2.23-2.21 (m, 3H), 2.09-2.07 (m, 2H), 1.75-1.73 (m, |
| | 4H). |

### Preparation 159: N-(3-(4-methylpiperazin-1-yl)-1H-pyrazolo[4,3-c]pyridin-6-yl)acetamide

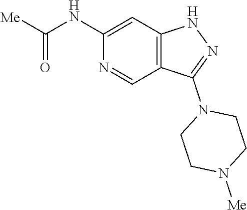 [SMILES: CC(=O)Nc1cc2[nH]nc(N3CCN(C)CC3)c2cn1]

A mixture of N-(3-(4-methylpiperazin-1-yl)-1-(tetrahydro-2H-pyran-2-yl)-1H-pyrazolo[4,3-c]pyridin-6-yl)acetamide (Preparation 155, 1.2 g, 3.35 mmol) in HCl/dioxane (4 M, 10 mL) was stirred at 10° C. for 12 h. The reaction mixture was concentrated and the residue purified by prep-HPLC-B (Gradient=0-15% MeCN) to give N-(3-(4-methylpiperazin-1-yl)-1H-pyrazolo[4,3-c]pyridin-6-yl)acetamide as a yellow solid (400 mg, 43%). ¹H NMR (500 MHz, MeOH-d₄) δ: 7.98 (s, 1H), 7.60 (s, 1H), 4.18-4.16 (m, 2H), 3.61-3.41 (m, 6H), 2.86 (s, 3H), 2.26 (s, 3H).

### Preparation 160: N-(3-(pyrrolidin-1-yl)-1H-pyrazolo[4,3-c]pyridin-6-yl)acetamide

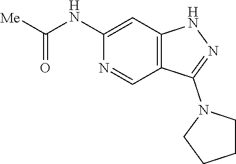 [SMILES: CC(=O)Nc1cc2[nH]nc(N3CCCC3)c2cn1]

N-(3-(Pyrrolidin-1-yl)-1H-pyrazolo[4,3-c]pyridin-6-yl)acetamide was prepared as a yellow oil (110 mg, crude) from N-(3-(pyrrolidin-1-yl)-1-(tetrahydro-2H-pyran-2-yl)-1H-pyrazolo[4,3-c]pyridin-6-yl)acetamide (Preparation 157) using an analogous method to that described for Preparation 159. LCMS m/z=246.2 [M+H]⁺.

### Preparation 161: N-(3-(3-cyanoazetidin-1-yl)-1H-pyrazolo[4,3-c]pyridin-6-yl)acetamide

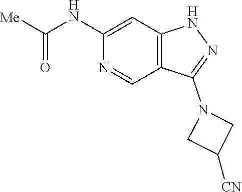 [SMILES: CC(=O)Nc1cc2[nH]nc(N3CC(C#N)C3)c2cn1]

TFA (595 mg, 5.22 mmol) was added to a solution of N-(3-(3-cyanoazetidin-1-yl)-1-(tetrahydro-2H-pyran-2-yl)-1H-pyrazolo[4,3-c]pyridin-6-yl)acetamide (Preparation 156, 120 mg, 0.353 mmol) in DCM (2 mL) and the mixture stirred at 20° C. for 12 h. The reaction mixture was evaporated to dryness to give N-(3-(3-cyanoazetidin-1-yl)-1H-pyrazolo[4,3-c]pyridin-6-yl)acetamide as a brown oil (150 mg, crude) which was used without further purification. LCMS m/z=256.9 [M+H]⁺.

### Preparation 162: N-(3-(4-methoxypiperidin-1-yl)-1H-pyrazolo[4,3-c]pyridin-6-yl)acetamide trifluoroacetate

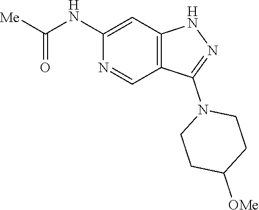 [SMILES: COC1CCN(c2n[nH]c3cc(NC(C)=O)ncc23)CC1]

N-(3-(4-methoxypiperidin-1-yl)-1H-pyrazolo[4,3-c]pyridin-6-yl)acetamide trifluoroacetate was prepared as a yellow oil (318 mg, crude) from N-(3-(4-methoxypiperidin-1-yl)-1-(tetrahydro-2H-pyran-2-yl)-1H-pyrazolo[4,3-c]pyridin-6-yl)acetamide (Preparation 158) using an analogous method to that described for Preparation 161. LCMS m/z=290.2 [M+H]⁺.

### Preparation 163: 3-(2-chloropyrimidin-4-yl)tetrahydrofuran-3-ol

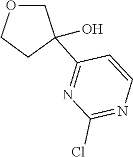 [SMILES: OC1(c2ccnc(Cl)n2)CCOC1]

n-BuLi (2.5 M, 20.9 mL) was added dropwise to a solution of 2,2,6,6-tetramethyl piperidine (7.4 g, 52.39 mmol) in THF (20 mL) at −70° C. The mixture was stirred at −70° C. for 30 min and 2-chloropyrimidine (2.0 g, 17.46 mmol) in THF (20.0 mL) was added dropwise the mixture stirred at −70° C. for 1 h. To this was slowly added dihydrofuran-3(2H)-one (6.1 g, 69.85 mmol) and stirring continued for 10 min before the temperature was warmed to 0° C. and the mixture stirred 1 h. The mixture was quenched with NH₄Cl (sat, 20 mL) and poured into H₂O (30 mL) and extracted with EtOAc (3×30 mL). The combined organics were dried (Na₂SO₄) and evaporated to dryness in vacuo. The residue was purified by chromatography (SiO₂, 25-50% EtOAc/PE) to give 3-(2-chloropyrimidin-4-yl)tetrahydrofuran-3-ol as a yellow oil (904.6 mg). ¹H NMR (500 MHz, CDCl₃) δ: 7.93 (d, 1H), 7.55 (d, 1H), 4.20-4.16 (m, 2H), 4.03-4.01 (m, 1H), 3.94-3.93 (m, 1H), 2.53-2.49 (m, 1H), 2.28-2.25 (m, 1H).

### Preparation 164: 3-(2-chloropyrimidin-4-yl)oxetan-3-ol

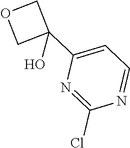 [SMILES: OC1(c2ccnc(Cl)n2)COC1]

3-(2-Chloropyrimidin-4-yl)oxetan-3-ol was prepared as a yellow solid (790 mg, 24%) from 3-(2-chloropyrimidin-4-yl)oxetan-3-ol (Preparation 164) and oxetan-3-one using an analogous method to that described for Preparation 163. ¹H NMR (400 MHz, CDCl₃) δ: 8.73 (d, 1H), 7.87 (d, 1H), 5.02-5.00 (m, 2H), 4.76-4.74 (m, 2H).

### Preparation 165: 3-(6-bromopyrimidin-4-yl)tetrahydrofuran-3-ol

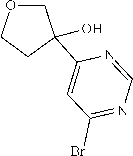 [SMILES: OC1(c2cc(Br)ncn2)CCOC1]

n-BuLi (2.5 M, 1.7 mL) was added to a solution of 4,6-dibromopyrimidine (1.0 g, 4.20 mmol) in DCM (10 mL) at −70° C. under N₂ and the mixture was stirred for 30 min. Dihydrofuran-3(2H)-one (362 mg, 4.20 mmol) was added to the solution and the mixture was stirred at −70° C. for 2 h. The mixture was quenched by sat. aq.NH₄Cl (3 mL), diluted with water (10 mL) and extracted with DCM (15 mL×3). The combined organics were washed with brine (20 mL) and evaporated to dryness. The residue was purified by column chromatography (PE/EtOAc=15/1 to 3/1) on silica gel to afford 3-(6-bromopyrimidin-4-yl)tetrahydrofuran-3-ol (305 mg, 30%) as colourless oil. ¹H NMR (400 MHz, MeOH-d₄) δ: 8.87 (s, 1H), 8.03 (s, 1H), 4.17-4.11 (m, 2H), 4.07-4.04 (m, 1H), 3.88-3.86 (m, 1H), 2.63-2.55 (m, 1H), 2.17-2.12 (m, 1H).

### Preparation 166: 2-chloro-4-(3-methoxytetrahydrofuran-3-yl)pyrimidine

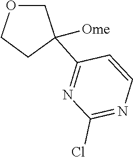

NaH (360.7 mg, 9.02 mmol, 60% purity) was added to a solution of 3-(2-chloropyrimidin-4-yl)tetrahydrofuran-3-ol (Preparation 163, 904.6 mg, 4.51 mmol) in THF (10 mL) at 0° C. and stirred for 10 mins. To this mixture was added CH₃I (960 mg, 6.76 mmol) and stirring continue at 25° C. for 12 h. The mixture was quenched with NH₄Cl (sat, 10 mL), poured into H₂O (20 mL) and extracted with EtOAc (3×20 mL). The combined organics was dried (Na₂SO₄) and evaporated to dryness in vacuo. The residue was purified by column chromatography (SiO₂, 20-50% EtOAc/PE) to give 2-chloro-4-(3-methoxytetrahydrofuran-3-yl)pyrimidine as a yellow oil (356 mg, 37%). ¹H NMR (500 MHz, CDCl₃) δ: 8.63 (d, 1H), 7.52 (d, 1H), 4.16-4.10 (m, 3H), 3.98-3.96 (m, 1H), 3.27 (s, 3H), 2.64-2.58 (m, 1H), 2.58-2.35 (m, 1H).

### Preparation 167: 4-bromo-6-(3-methoxytetrahydrofuran-3-yl)pyrimidine

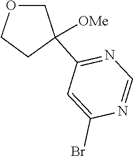 [SMILES: COC1(c2cc(Br)ncn2)CCOC1]

4-Bromo-6-(3-methoxytetrahydrofuran-3-yl)pyrimidine was prepared as a colourless oil (190 mg, 90%) from 3-(6-bromopyrimidin-4-yl)tetrahydrofuran-3-ol (Preparation 165) using an analogous method to that described for Preparation 166. ¹H NMR (400 MHz, MeOH-d₄) δ: 8.90 (s, 1H), 7.90 (d, 1H), 4.13-4.04 (m, 4H), 3.28 (s, 3H), 3.59-3.52 (m, 1H), 2.44-2.41 (m, 1H).

### Preparation 168: 2-chloro-4-(3-fluorotetrahydrofuran-3-yl)pyrimidine

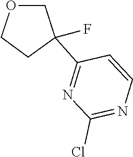 [SMILES: FC1(c2ccnc(Cl)n2)CCOC1]

DAST (161 mg, 0.997 mmol) was added to a solution of 3-(2-chloropyrimidin-4-yl)tetrahydrofuran-3-ol (Preparation 163, 100 mg, 0.498 mmol) in DCM (5 mL) and the mixture was stirred at 25° C. for 10 h. The mixture was quenched with aq. NH₄Cl (30 mL) and extracted with DCM (3×10 mL). The combined organics were evaporated to dryness and the residue purified by column chromatography (SiO₂, 20% EtOAc/PE to give 2-chloro-4-(3-fluorotetrahydrofuran-3-yl)pyrimidine as a yellow oil (47.3 mg, 47%). ¹H NMR (400 MHz, CDCl₃) δ: 8.71-8.67 (m, 1H), 7.61-7.59 (m, 1H), 4.24-4.19 (m, 3H), 4.18-4.09 (m, 1H), 2.75-2.63 (m, 1H), 2.48-2.38 (m, 1H).

### Preparation 169: 2-chloro-4-(3-fluorooxetan-3-yl)pyrimidine

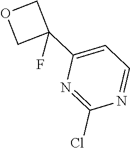 [SMILES: FC1(c2ccnc(Cl)n2)COC1]

2-Chloro-4-(3-fluorooxetan-3-yl)pyrimidine was prepared as a yellow oil (390 mg, 52%) from 3-(2-chloropyrimidin-4-yl)oxetan-3-ol (Preparation 164) using an analogous method to that described for Preparation 168. ¹H NMR (400 MHz, CDCl₃) δ: 8.70 (d, 1H), 7.48 (d, 1H), 5.15-5.07 (m, 2H), 5.03-4.95 (m, 2H).

### Preparation 170: 2-chloro-4-(furan-3-yl)pyrimidine

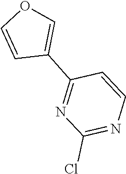 [SMILES: Clc1nccc(-c2ccoc2)n1]

A mixture of 2,4-dichloropyrimidine (2.0 g, 13.42 mmol), furan-3-ylboronic acid (1.5 g, 13.42 mmol), Pd(dppf)Cl₂ (982.3 mg, 1.34 mmol), K₂CO₃ (3.7 g, 26.85 mmol) in dioxane (25 mL) and H₂O (5 mL) was stirred at 100° C. for 2 h under N₂. The mixture was concentrated under reduced pressure and the residue purified by chromatography on silica gel (PE/EtOAc=3/1) to give 2-chloro-4-(furan-3-yl)pyrimidine as a yellow oil (700 mg, 29%). ¹H NMR (400 MHz, CDCl₃) δ: 8.55 (d, 1H), 8.22 (s, 1H), 7.55-7.52 (m, 1H), 7.31 (d, 1H), 6.90 (s, 1H).

### Preparation 171: N-(3-cyclopropyl-1-(4-(furan-3-yl)pyrimidin-2-yl)-1H-pyrazolo[4,3-c]pyridin-6-yl)acetamide

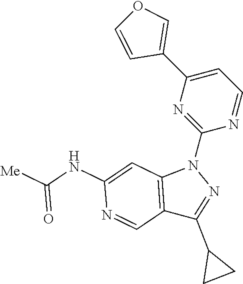 [SMILES: CC(=O)Nc1cc2c(cn1)c(C1CC1)nn2-c1nccc(-c2ccoc2)n1]

A mixture of 2-chloro-4-(furan-3-yl)pyrimidine (Preparation 170, 100 mg, 0.554 mmol), N-(3-cyclopropyl-1H-pyrazolo[4,3-c]pyridin-6-yl)acetamide (Preparation 11, 120 mg, 0.554 mmol) and Cs₂CO₃ (360.8 mg, 1.11 mmol) in DMF (5 mL) was stirred at 60° C. for 2 h. The mixture was concentrated and the residue purified by chromatography on silica gel (PE/EtOAc=1/1) to give N-(3-cyclopropyl-1-(4-(furan-3-yl)pyrimidin-2-yl)-1H-pyrazolo[4,3-c]pyridin-6-yl)acetamide as a yellow solid (70 mg, 35%). ¹H NMR (400 MHz, CDCl₃) δ: 9.65 (s, 1H), 8.87 (br s, 1H), 8.80-8.77 (m, 2H), 8.72 (s, 1H), 7.62 (s, 1H), 7.28 (s, 1H), 7.18 (s, 1H), 2.41-2.36 (m, 1H), 2.33 (s, 3H), 1.33-1.31 (m, 2H), 1.22-1.19 (m, 2H).

### Preparation 172: 1-(3-chloropyrazin-2-yl)ethan-1-one

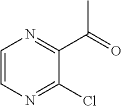

K₂S₂O₈ (11.8 g, 43.66 mmol) and AgNO₃ (860.2 mg, 5.06 mmol) were added to a solution of 2-chloropyrazine (1.0 g, 8.73 mmol) and 2-oxopropanoic acid (768.9 mg, 8.73 mmol) in DCM (10 mL) and H₂O (10 mL) at 25° C. and the mixture stirred at 25° C. for 16 h. The mixture was filtered and the filtrated was treated with H₂O (20 mL) and extracted with DCM (3×15 mL). The combined organics were washed with brine (20 mL), dried (Na₂SO₄) and evaporated to dryness in vacuo. The residue was purified by column chromatography (SiO₂, 6-50% EtOAc/PE) to give 1-(3-chloropyrazin-2-yl)ethan-1-one as a pale yellow solid (150 mg, 11%). ¹H NMR (500 MHz, CDCl₃) δ: 8.56 (d, 1H), 8.51 (d, 1H), 2.71 (s, 3H).

### Preparation 173: 2-chloro-3-(1,1-difluoroethyl)pyrazine

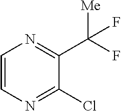 [SMILES: CC(F)(F)c1nccnc1Cl]

DAST (154.4 mg, 0.958 mmol) was added to a solution of 1-(3-chloropyrazin-2-yl)ethan-1-one (Preparation 172, 150 mg, 0.958 mmol) in DCM (5 mL) at 25° C. and the mixture was stirred at 25° C. for 24 h. The mixture was concentrated under reduced pressure and the residue purified by prep-TLC (25% EtOAc/PE) to give 2-chloro-3-(1,1-difluoroethyl)pyrazine as a yellow oil (34 mg, 20%). ¹H NMR (500 MHz, CDCl₃) δ: 8.51 (d, 1H), 8.48 (d, 1H), 2.15-2.07 (m, 3H).

### Preparation 174: 6-chloro-N-methoxy-N-methylpyrazine-2-carboxamide

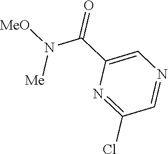 [SMILES: CON(C)C(=O)c1cncc(Cl)n1]

To a solution of 6-chloropyrazine-2-carboxylic acid (5.0 g, 31.5 mmol) in DCM (50 mL) was added (COCl)₂ (4.8 g, 37.84 mmol, 3.20 mL), DMF (0.2 mL) and the mixture stirred at 10° C. for 12 h. To this was added N,O-dimethylhydroxylamine hydrochloride (4.0 g, 41 mmol) and then TEA (9.6 g, 94.6 mmol) dropwise. The resulting mixture was stirred at 10° C. for 2 h, diluted with H₂O (20 mL) and extracted with DCM (3×20 mL). The combined organics were washed with brine (2×20 mL), dried (Na₂SO₄) and evaporated to dryness in vacuo. The residue was purified by column chromatography (SiO₂, 25% EtOAc/PE) to give 6-chloro-N-methoxy-N-methylpyrazine-2-carboxamide as a yellow oil (3.2 g, 50%). ¹H NMR (500 MHz, CDCl₃) δ: 8.78 (br s, 1H), 8.67 (s, 1H), 3.78 (s, 3H), 3.38 (s, 3H).

### Preparation 175: 1-(6-chloropyrazin-2-yl)ethan-1-one

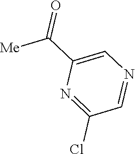

To a solution of 6-chloro-N-methoxy-N-methylpyrazine-2-carboxamide (Preparation 174, 3.2 g, 15.9 mmol) in THF (30 mL) was added dropwise MeMgBr (3 M, 5.29 mL) at 0° C. under N₂. The resulting mixture was allowed to warm to rt and was stirred for 2 h. The reaction was quenched with NH₄Cl (10 mL) and extracted with EtOAc (2×20 mL). The combined organics were washed with brine (2×15 mL), dried (Na₂SO₄) and evaporated to dryness in vacuo. The residue was purified by chromatography (SiO₂, 25% EtOAc/PE) to give 1-(6-chloropyrazin-2-yl)ethan-1-one as a yellow oil (1.2 g, 48%). ¹H NMR (400 MHz, CDCl₃) δ: 9.10 (s, 1H), 8.77 (s, 1H), 2.70 (s, 3H).

### Preparation 176: 2-chloro-6-(1,1-difluoroethyl)pyrazine

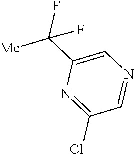 [SMILES: CC(F)(F)c1cncc(Cl)n1]

To a solution of 1-(6-chloropyrazin-2-yl)ethan-1-one (Preparation 175, 1.2 g, 7.66 mmol) in DCM (10 mL) was added DAST (2.5 g, 15.33 mmol) and the resulting mixture was stirred at 10° C. for 12 h. The mixture was quenched with H₂O (10 mL) and extracted with DCM (2×15 mL). The combined organics were washed with brine (2×10 mL), dried (Na₂SO₄) and evaporated to dryness in vacuo. The residue was purified by chromatography (SiO₂, 16% EtOAc/PE) to give 2-chloro-6-(1,1-difluoroethyl)pyrazine as a yellow oil (420 mg, 31%). ¹H NMR (500 MHz, CDCl₃) δ: 8.84 (s, 1H), 8.70 (s, 1H), 2.04 (t, 3H).

### Preparation 177: 1-(2-chloropyrimidin-4-yl)ethan-1-one

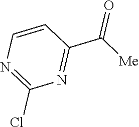

To a solution of 2-chloropyrimidine (1 g, 8.73 mmol), 2-oxopropanoic acid (769 mg, 8.73 mmol) and AgNO₃ (297 mg, 1.75 mmol) in DCM (5 mL) and H₂O (5 mL) was added K₂S₂O₈ (4.7 g, 17.46 mmol) and the resulting mixture stirred at 30° C. for 16 h. The mixture was extracted with DCM (3×10 mL) and the combined organics washed brine (2×10 mL), dried (Na₂SO₄) and evaporated to dryness in vacuo. The residue was purified by column chromatography (SiO2, 16-25% EtOAc/PE) to afford 1-(2-chloropyrimidin-4-yl)ethan-1-one as a yellow solid (70 mg, 5%). ¹H NMR (400 MHz, CDCl₃) δ: 8.86 (d, 1H), 7.84 (d, 1H), 2.72 (s, 3H).

### Preparation 178: 2-chloro-4-(1,1-difluoroethyl)pyrimidine

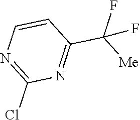 [SMILES: CC(F)(F)c1ccnc(Cl)n1]

2-Chloro-4-(1,1-difluoroethyl)pyrimidine was prepared as a yellow oil (40 mg, 70%) from 1-(2-chloropyrimidin-4-yl)ethan-1-one (Preparation 177) using an analogous method to that described for Preparation 176. ¹H NMR (500 MHz, CDCl₃) δ: 8.82-8.76 (m, 1H), 7.61-7.54 (m, 1H), 2.00 (t, J=19.0 Hz, 3H).

### Preparation 179: 1-(2-chloro-6-methylpyrimidin-4-yl)ethan-1-one

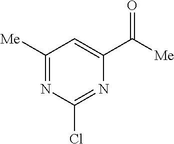 [SMILES: CC(=O)c1cc(C)nc(Cl)n1]

MeMgBr (3M, 1.76 mL) was added to a solution of methyl 2-chloro-6-methylpyrimidine-4-carboxylate (985 mg, 5.28 mmol) in THF (10 mL) at 0° C. under N₂ atmosphere and stirred for 5 h. The reaction was quenched with H₂O (30 mL) and extracted with ethyl acetate (3×20 mL). The combined organics were dried (Na₂SO₄), evaporated to dryness and the residue purified by silica gel chromatography (16% EtOAc/PE) PE/ethyl acetate=5/1) to give 1-(2-chloro-6-methylpyrimidin-4-yl)ethan-1-one as a white solid (147 mg, 16%). ¹H NMR (400 MHz, CDCl₃) δ: 7.69 (s, 1H), 2.70 (s, 3H), 2.63 (s, 3H).

### Preparation 180: 2-chloro-4-(1,1-difluoroethyl)-6-methylpyrimidine

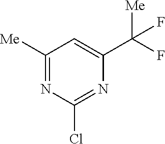 [SMILES: Cc1cc(C(C)(F)F)nc(Cl)n1]

2-Chloro-4-(1,1-difluoroethyl)-6-methylpyrimidine was prepared as a yellow oil from 1-(2-chloro-6-methylpyrimidin-4-yl)ethan-1-one (Preparation 179) using an analogous method to that described for Preparation 176.

### Preparation 181: 1-(4-(benzyloxy)pyrimidin-2-yl)ethan-1-one

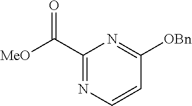 [SMILES: COC(=O)c1nccc(OCc2ccccc2)n1]

To a solution of methyl 4-(benzyloxy)pyrimidine-2-carboxylate (1.5 g, 6.14 mmol) in THE (20 mL) was added dropwise CH₃MgBr (3 M, 2.05 mL) at 0° C. The mixture was stirred for 1 h and then at 25° C. for 12 h under N₂. The mixture was quenched with NH₄Cl (sat. 20 mL), poured into H₂O (30 mL) and extracted with EtOAc (3×30 mL). The combined organics were dried (Na₂SO₄) and evaporated to dryness in vacuo. The residue was purified by chromatography (PE/EtOAc=3/1) on silica gel to give 1-(4-(benzyloxy)pyrimidin-2-yl)ethan-1-one as a colourless oil (288 mg, 21%) as colorless oil. ¹H NMR (400 MHz, CDCl₃) δ: 8.61 (d, 1H), 7.49-7.35 (m, 5H), 6.90 (d, 1H), 5.52 (s, 2H), 2.75 (s, 3H).

### Preparation 182: 4-(benzyloxy)-2-(1,1-difluoroethyl)pyrimidine

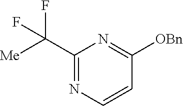 [SMILES: CC(F)(F)c1nccc(OCc2ccccc2)n1]

To a solution of 1-(4-(benzyloxy)pyrimidin-2-yl)ethan-1-one (Preparation 181, 640 mg, 2.80 mmol) in DCM (5 mL) was added DAST (904 mg, 5.61 mmol) at 0° C. and the resulting mixture stirred at 25° C. for 12 h. The mixture was quenched with Na₂CO₃ (10 mL), poured into H₂O (20 mL) and extracted with EtOAc (2×20 mL). The combined organics were washed with brine (2×15 mL), dried (Na₂SO₄) and evaporated to dryness under reduced pressure. The residue was purified by chromatography on silica gel (PE/EtOAc=3/1) to give 4-(benzyloxy)-2-(1,1-difluoroethyl)pyrimidine as a yellow oil (537 mg, 76%). ¹H NMR (500 MHz, CDCl₃) δ: 8.52 (d, 1H), 7.48-7.35 (m, 5H), 6.81 (d, 1H), 5.48 (s, 2H), 2.03 (t, 3H).

### Preparation 183: 2-(1,1-difluoroethyl)pyrimidin-4-ol

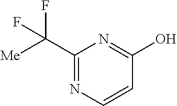 [SMILES: CC(F)(F)c1nccc(O)n1]

A solution of 4-(benzyloxy)-2-(1,1-difluoroethyl)pyrimidine (Preparation 182, 537.1 mg, 2.15 mmol) in TFA (4.47 g, 39.2 mmol) was stirred at 100° C. for 12 h. The mixture was purified by prep-HPLC-D (0-20% MeCN) to give 2-(1,1-difluoroethyl)pyrimidin-4-ol as a white solid (230 mg, 67%). ¹H NMR (400 MHz, CDCl₃) δ: 7.99 (d, 1H), 6.55 (d, 1H), 2.03 (t, 3H).

### Preparation 184: 4-chloro-2-(1,1-difluoroethyl)pyrimidine

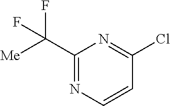 [SMILES: CC(F)(F)c1nccc(Cl)n1]

To a solution of 2-(1,1-difluoroethyl)pyrimidin-4-ol (Preparation 183, 100 mg, 0.625 mmol) in POCl₃ (2 mL) was stirred at 100° C. for 2 h. The reaction mixture was concentrated and was added to H₂O (10 mL) and extracted with EtOAc (3×10 mL). The combined organics were dried (Na₂SO₄) and concentrated to give 4-chloro-2-(1,1-difluoroethyl)pyrimidine as a yellow oil (85 mg, 76%). ¹H NMR (400 MHz, CDCl₃) δ: 8.74 (d, 1H), 7.44 (d, 1H), 2.07 (t, 3H).

### Preparation 185: 2-(4-(benzyloxy)pyrimidin-2-yl)propan-2-ol

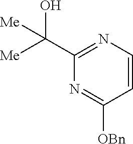 [SMILES: CC(C)(O)c1nccc(OCc2ccccc2)n1]

To a solution of methyl 4-(benzyloxy)pyrimidine-2-carboxylate (1.5 g, 6.14 mmol) in THE (20 mL) was added dropwise CH₃MgBr (3 M, 2.05 mL) at 0° C. The mixture was stirred for 1 h, then warmed 25° C. for 12 h under N₂. The mixture was quenched with NH₄Cl (sat. 20 mL), poured into H₂O (30 mL) and extracted with EtOAc (3×30 mL). The combined organics were dried (Na₂SO₄) and evaporated to dryness in vacuo. The residue was purified by chromatography (PE/EtOAc=3/1) on silica gel to give 2-(4-(benzyloxy)pyrimidin-2-yl)propan-2-ol as a white solid (358 mg, 24%). ¹H NMR (400 MHz, CDCl₃) δ: 8.40 (d, 1H), 7.46-7.34 (m, 5H), 6.65 (d, 1H), 5.44 (s, 2H), 4.68 (s, 1H), 1.56 (s, 6H).

### Preparation 186: 4-(benzyloxy)-2-(2-fluoropropan-2-yl)pyrimidine

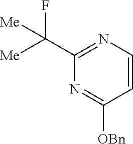 [SMILES: CC(C)(F)c1nccc(OCc2ccccc2)n1]

4-(Benzyloxy)-2-(2-fluoropropan-2-yl)pyrimidine was obtained, (556 mg, 79%) as yellow oil, from 2-(4-(benzyloxy)pyrimidin-2-yl)propan-2-ol (Preparation 185) following an analogous procedure to that described in Preparation 182. ¹H NMR (500 MHz, CDCl₃) δ: 8.48-8.46 (m, 1H), 7.46-7.33 (m, 5H), 6.67 (d, 1H), 5.45 (s, 2H), 1.79 (s, 3H), 1.74 (s, 3H).

### Preparation 187: 2-(2-fluoropropan-2-yl)pyrimidin-4-ol

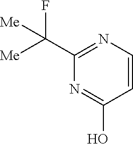 [SMILES: CC(C)(F)c1nccc(O)n1]

To a solution of 4-(benzyloxy)-2-(2-fluoropropan-2-yl)pyrimidine (Preparation 186, 556 mg, 2.26 mmol) in TFA (4.47 g, 39.2 mmol) was stirred at 100° C. for 12 h. The mixture was purified by prep-HPLC-E (0-30% MeCN) to give 2-(2-fluoropropan-2-yl)pyrimidin-4-ol (258 mg, 73%) as a white solid. ¹H NMR (500 MHz, CDCl₃) δ: 7.91 (d, 1H), 6.36 (d, 1H), 1.76 (s, 3H), 1.71 (s, 3H).

### Preparation 188: 4-chloro-2-(2-fluoropropan-2-yl)pyrimidine

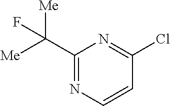 [SMILES: CC(C)(F)c1nccc(Cl)n1]

To a solution of 2-(2-fluoropropan-2-yl)pyrimidin-4-ol (Preparation 187, 100 mg, 0.64 mmol) in POCl₃ (2 mL) was stirred at 100° C. for 2 h. The mixture was concentrated and was added to H₂O (10 mL) and extracted with EtOAc (3×10 mL). The combined organics were dried over anhydrous Na₂SO₄, filtered and concentrated to give 4-chloro-2-(2-fluoropropan-2-yl)pyrimidine (82 mg, 73%) as colorless oil. ¹H NMR (500 MHz, CDCl₃) δ: 8.66 (d, 1H), 7.29 (d, 1H), 1.81 (s, 3H), 1.77 (s, 3H).

### Preparation 189: 4,6-dichloro-2-(1,1-difluoroethyl)pyrimidine

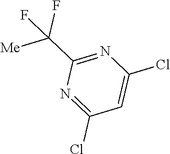 [SMILES: CC(F)(F)c1nc(Cl)cc(Cl)n1]

Part 1: Na (1.1 g, 48.27 mmol) in EtOH (20 mL) was stirred at 60° C. for 2 h. To this solution was added ethyl 2,2-difluoropropanoate (5 g, 36.20 mmol) and malonamide (2.46 g, 24.13 mmol) and the resulting mixture was stirred at 100° C. for 12 h. The mixture was concentrated and the residue was diluted with H₂O (10 mL) and extracted with EtOAc (20 mL×2). The aqueous phase was adjusted pH to 1-3 with aq. HCl and the mixture concentrated under reduced pressure to give 2-(1,1-difluoroethyl)pyrimidine-4,6-diol as a yellow solid (3 g) which was used without further purification. ¹H NMR (500 MHz, MeOH-d₄) δ: 5.71 (s, 1H), 1.69 (t, 3H). Part 2: A mixture of 2-(1,1-difluoroethyl)pyrimidine-4,6-diol (Part 1, 3.0 g, 17.03 mmol) in POCl₃ (24.7 g, 161 mmol) was stirred at 80° C. for 3 h. The mixture was poured into H₂O (40 mL) and extracted with EtOAc (30 mL×3). The combined organic phase was washed with brine (20 mL×2), dried Na₂SO₄) and evaporated to dryness. The residue was purified by chromatography on silica gel (PE/EtOAc=5/1) to give 4,6-dichloro-2-(1,1-difluoroethyl)pyrimidine as a colourless oil (1.0 g, 28%). ¹H NMR (500 MHz, CDCl₃) δ: 7.49 (s, 1H), 2.05 (t, 3H).

### Preparation 190: 4-chloro-2-(1,1-difluoroethyl)-6-methoxypyrimidine

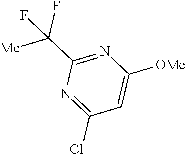 [SMILES: COc1cc(Cl)nc(C(C)(F)F)n1]

MeONa (101.5 mg, 1.88 mmol) was added to a solution of 4,6-dichloro-2-(1,1-difluoroethyl)pyrimidine (Preparation 189, 400 mg, 1.88 mmol) in MeOH (5 mL) and the resulting mixture stirred at 15° C. for 2 h. The reaction was concentrated under reduced pressure and the residue was purified by chromatography on silica gel (PE/EtOAc=10/1) to give 4-chloro-2-(1,1-difluoroethyl)-6-methoxypyrimidine as a colourless oil (300 mg, 77%). ¹H NMR (500 MHz, CDCl₃) δ: 6.81 (s, 1H), 4.05 (s, 3H), 2.02 (t, 3H).

### Preparation 191: 3-(4-chloropyrimidin-2-yl)oxetan-3-ol

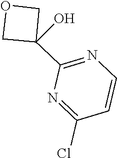 [SMILES: OC1(c2nccc(Cl)n2)COC1]

To a solution of 2-bromo-4-chloropyrimidine (1.0 g, 5.17 mmol) in toluene (10 mL) was added dropwise n-BuLi (2.5 M, 2.17 mL) at −70° C. over 30 minutes under N₂. To this was added oxetan-3-one (410 mg, 5.69 mmol) slowly and the resulting mixture was allowed to warm up to rt and stirred for 3 h. The mixture was quenched with H₂O (10 mL) and extracted with EtOAc (20 mL×2). The combined organic phase was washed with brine (10 mL×2), dried (Na₂SO₄) and evaporated to dryness in vacuo. The residue was purified by chromatography on silica gel (50-100% EtOAc/PE) to give 3-(4-chloropyrimidin-2-yl)oxetan-3-ol as a yellow oil (320 mg, 33%). ¹H NMR (500 MHz, CDCl₃) δ: 8.71 (d, 1H), 7.36 (d, 1H), 5.04-5.02 (m, 2H), 4.99-4.97 (m, 1H).

### Preparation 192: 3-(4-chloropyrimidin-2-yl)tetrahydrofuran-3-ol

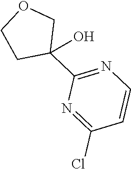 [SMILES: OC1(c2nccc(Cl)n2)CCOC1]

3-(4-Chloropyrimidin-2-yl)tetrahydrofuran-3-ol was prepared as a brown oil (653 mg, 35%) from 2-bromo-4-chloropyrimidine and dihydrofuran-3(2H)-one using an analogous method to that described for Preparation 191. ¹H NMR (400 MHz, CDCl₃) δ: 8.63 (d, 1H), 7.30 (d, 1H), 4.61 (s, 1H), 4.22-4.16 (m, 3H), 3.98-3.96 (m, 1H), 2.67-2.59 (m, 1H), 2.26-2.22 (m, 1H).

### Preparation 193: 4-chloro-2-(3-fluorooxetan-3-yl)pyrimidine

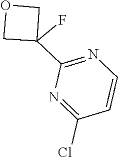 [SMILES: FC1(c2nccc(Cl)n2)COC1]

4-Chloro-2-(3-fluorooxetan-3-yl)pyrimidine was obtained as a yellow oil, 60 mg, 19%, from 3-(4-chloropyrimidin-2-yl)oxetan-3-ol (Preparation 191), following an analogous procedure to that described in Preparation 176. ¹H NMR (500 MHz, CDCl₃) δ: 8.73 (d, 1H), 7.39 (d, 1H), 5.21-5.19 (m, 1H), 5.16-5.15 (m, 1H), 5.14-5.11 (m, 1H), 5.10-5.07 (m, 1H).

### Preparation 194: 4-chloro-2-(3-fluorotetrahydrofuran-3-yl)pyrimidine

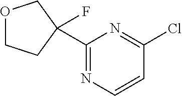 [SMILES: FC1(c2nccc(Cl)n2)CCOC1]

4-Chloro-2-(3-fluorotetrahydrofuran-3-yl)pyrimidine was prepared as a yellow oil (143 mg, 46%) from 3-(4-chloropyrimidin-2-yl)tetrahydrofuran-3-ol (Preparation 192) using an analogous method to that described for Preparation 176. ¹H NMR (500 MHz, CDCl₃) δ: 8.67 (d, 1H), 7.33 (d, 1H), 4.35-4.18 (m, 4H), 2.77-2.72 (m, 1H), 2.71-2.57 (m, 1H).

### Preparation 195: 4-(benzyloxy)-2-(2-methoxypropan-2-yl)pyrimidine

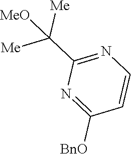 [SMILES: COC(C)(C)c1nccc(OCc2ccccc2)n1]

A solution of 2-(4-(benzyloxy)pyrimidin-2-yl)propan-2-ol (Preparation 185, 1.6 g, 6.43 mmol) and NaH (514 mg, 12.85 mmol, 60% purity) in THF (10 mL) was stirred at 25° C. for 30 mins. Then MeI (1.8 g, 12.85 mmol) was added and the mixture stirred at 25° C. for 12 h. The mixture was quenched with H₂O (20 mL) and extracted with EtOAc (20 mL×3). The combined organics were evaporated to dryness in vacuo and the residue purified by silica gel chromatography (PE/EtOAc=2/1) to give 4-(benzyloxy)-2-(2-methoxypropan-2-yl)pyrimidine as a yellow oil (1.4 g, 82%). ¹H NMR (400 MHz, CDCl₃) δ: 8.48-8.44 (m, 1H), 7.46 (d, 2H), 7.39-7.33 (m, 3H), 6.64 (d, 1H), 5.45 (s, 2H), 3.19 (s, 3H), 1.61 (s, 3H).

### Preparation 196: 2-(2-methoxypropan-2-yl)pyrimidin-4-ol

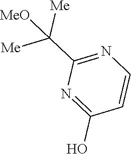 [SMILES: COC(C)(C)c1nccc(O)n1]

A solution of 4-(benzyloxy)-2-(2-methoxypropan-2-yl)pyrimidine (Preparation 195, 1.3 g, 4.92 mmol) in TFA (10 mL) was stirred at 100° C. for 10 h. The mixture was concentrated in vacuum and purified by silica gel chromatography (PE/EtOAc=1/2) to give 2-(2-methoxypropan-2-yl)pyrimidin-4-ol as a grey solid (1.1 g, 138%). ¹H NMR (400 MHz, CDCl₃) δ: 7.94 (d, 1H), 6.35 (d, 1H), 3.32 (s, 3H), 1.53 (s, 6H).

### Preparation 197: 4-chloro-2-(2-methoxypropan-2-yl)pyrimidine

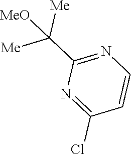 [SMILES: COC(C)(C)c1nccc(Cl)n1]

A solution of 2-(2-methoxypropan-2-yl)pyrimidin-4-ol (Preparation 196, 0.2 g, 1.19 mmol) in POCl₃ (4 mL) was stirred at 100° C. for 12 h. The mixture was added to H₂O (30 mL) and extracted with DCM (20 mL×3). The combined organics were dried (Na₂SO₄) and evaporated to dryness in vacuo to give 4-chloro-2-(2-methoxypropan-2-yl)pyrimidine as a yellow oil (90 mg, 41%) which was used without further purification.

### Preparation 198: 0-(3-(4-chloropyrimidin-2-yl)tetrahydrofuran-3-yl) S-methyl carbonodithioate

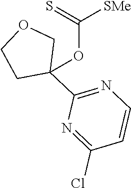 [SMILES: CSC(=S)OC1(c2nccc(Cl)n2)CCOC1]

A solution of 3-(4-chloropyrimidin-2-yl)tetrahydrofuran-3-ol (Preparation 192, 600 mg, 2.99 mmol) in THF (8 mL) was added NaH (359 mg, 8.97 mmol, 60% purity) and the mixture stirred for 30 mins. CS₂ (227.7 mg, 2.99 mmol) was added and stirred for 30 mins followed by addition of CH₃I (424.5 mg, 2.99 mmol) and the resulting mixture was stirred 50° C. for 2 h. The mixture was quenched with H₂O (20 mL) and extracted with EtOAc (20 mL×3). The organic phase was washed by brine (20 mL), dried (Na₂SO₄) and concentrated. The residue was purified by chromatography (PE/EtOAc=20/1 to 3/1) on silica gel to give O-(3-(4-chloropyrimidin-2-yl)tetrahydrofuran-3-yl) S-methyl carbonodithioate as a colourless oil (120.7 mg, 14%). ¹H NMR (400 MHz, CDCl₃) δ: 8.57 (d, 1H), 7.20 (d, 1H), 4.57-4.55 (m, 1H), 4.48-4.45 (m, 1H), 4.14-4.10 (m, 2H), 2.90-2.85 (m, 1H), 2.82-2.77 (m, 1H), 2.54 (s, 3H).

### Preparation 199: 0-(3-(4-chloropyrimidin-2-yl)oxetan-3-yl) S-methyl carbonodithioate

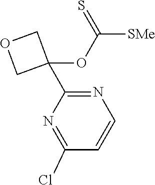 [SMILES: CSC(=S)OC1(c2nccc(Cl)n2)COC1]

O-(3-(4-chloropyrimidin-2-yl)oxetan-3-yl) S-methyl carbonodithioate was prepared as a yellow oil (450 mg, 38%) from 3-(4-chloropyrimidin-2-yl)oxetan-3-ol (Preparation 191) using an analogous method to that described for Preparation 198. ¹H NMR (400 MHz, CDCl₃) δ: 8.55 (d, 1H), 7.21 (d, 1H), 5.20-5.18 (m, 2H), 5.13-5.11 (m, 2H), 2.59 (s, 3H).

### Preparation 200: 4-chloro-2-(tetrahydrofuran-3-yl)pyrimidine

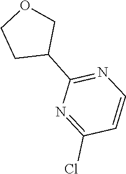 [SMILES: Clc1ccnc(C2CCOC2)n1]

To a solution of O-(3-(4-chloropyrimidin-2-yl)tetrahydrofuran-3-yl) S-methyl carbonodithioate (Preparation 198, 120 mg, 0.413 mmol) in toluene (3 mL) was added AIBN (6.8 mg, 0.041 mmol) and tributyltin (240.2 mg, 0.825 mmol) and the resulting mixture stirred at 125° C. for 1 h under N₂. The mixture was quenched with aq. KF (10 mL) and extracted with EtOAc (10 mL×3). The combined organics were washed with brine (10 mL), dried (Na₂SO₄) and evaporated to dryness in vacuo. The residue was concentrated and purified by chromatography (PE/EtOAc=20/1 to 3/1) on silica gel to give 4-chloro-2-(tetrahydrofuran-3-yl)pyrimidine as a colourless oil (52.5 mg, 69%). ¹H NMR (400 MHz, CDCl₃) δ: 8.56 (d, 1H), 7.21 (d, 1H), 4.24-4.19 (m, 1H), 4.07-4.03 (m, 2H), 3.96-3.94 (m, 1H), 3.76-3.74 (m, 1H), 2.41-2.35 (m, 2H).

### Preparation 201: 4-chloro-2-(oxetan-3-yl)pyrimidine

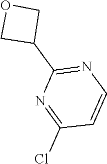 [SMILES: Clc1ccnc(C2COC2)n1]

4-Chloro-2-(oxetan-3-yl)pyrimidine was prepared as a yellow oil (105 mg, 38%) from 0-(3-(4-chloropyrimidin-2-yl)oxetan-3-yl) S-methyl carbonodithioate (Preparation 199) using an analogous method to that described for Preparation 200. ¹H NMR (400 MHz, CDCl₃) δ: 8.63 (d, 1H), 7.27-7.26 (m, 1H), 5.07-5.01 (m, 4H), 4.52-4.46 (m, 1H).

### Preparation 202: 2-(4,6-dichloropyrimidin-2-yl)propan-2-ol

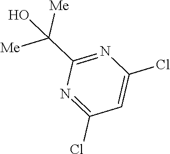 [SMILES: CC(C)(O)c1nc(Cl)cc(Cl)n1]

To a solution of 4,6-dichloro-2-iodopyrimidine (1 g, 3.64 mmol) in toluene (10 mL) was added n-BuLi (2.5 M, 1.46 mL) at −78° C. for 30 minutes under N₂ before acetone (211 mg, 3.64 mmol) was added slowly. The resulting mixture was allowed to warm up to rt for 2 h. The reaction was quenched with NH₄Cl (10 mL) and extracted with EtOAc (20 mL×3). The combined organics were washed with brine (10 mL×2), dried (Na₂SO₄) and evaporated to dryness in vacuo. The residue was purified by chromatography on silica gel (PE/EtOAc=5/1 to 3/1) to give 2-(4,6-dichloropyrimidin-2-yl)propan-2-ol (500 mg, 66%) as a yellow oil. ¹H NMR (500 MHz, CDCl₃) δ: 7.30 (s, 1H), 3.92 (br s, 1H), 1.59 (s, 6H).

### Preparation 203: 3-(4,6-dichloropyrimidin-2-yl)oxetan-3-ol

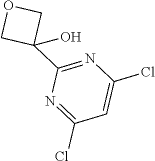 [SMILES: OC1(c2nc(Cl)cc(Cl)n2)COC1]

3-(4,6-Dichloropyrimidin-2-yl)oxetan-3-ol was prepared as a yellow solid (380 mg, 47%) from 4,6-dichloro-2-iodopyrimidine and oxetan-3-one using an analogous method to that described for Preparation 202. ¹H NMR (500 MHz, CDCl₃) δ: 7.40 (s, 1H), 5.03 (d, 2H), 4.94 (d, 2H), 4.60 (br s, 1H).

### Preparation 204: 3-(4,6-dichloropyrimidin-2-yl)tetrahydrofuran-3-ol

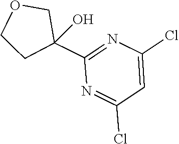 [SMILES: OC1(c2nc(Cl)cc(Cl)n2)CCOC1]

3-(4,6-Dichloropyrimidin-2-yl)tetrahydrofuran-3-ol was prepared as a yellow oil (1.72 g, 50%) from 4,6-dichloro-2-iodopyrimidine and dihydrofuran-3(2H)-one using an analogous method to that described for Preparation 202. ¹H NMR (400 MHz, CDCl₃) δ: 7.34 (s, 1H), 4.24-4.15 (m, 4H), 3.98-3.96 (m, 1H), 2.64-2.59 (m, 1H), 2.26-2.22 (m, 1H).

### Preparation 205: 3-(4-chloro-6-methoxypyrimidin-2-yl)tetrahydrofuran-3-ol

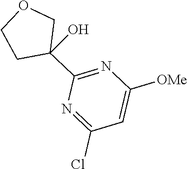 [SMILES: COc1cc(Cl)nc(C2(O)CCOC2)n1]

To a solution of 3-(4,6-dichloropyrimidin-2-yl)tetrahydrofuran-3-ol (Preparation 204, 1.6 g, 6.81 mmol) in MeOH (20 mL) was added NaOMe (735 mg, 13.61 mmol) and the resulting mixture stirred at 20° C. for 3 h. The mixture was concentrated and the residue was purified by chromatography (PE/EtOAc=20/1 to 3/1) on silica gel to give 3-(4-chloro-6-methoxypyrimidin-2-yl)tetrahydrofuran-3-ol as a colourless oil (1.3 g, 82%). ¹H NMR (400 MHz, CDCl₃) δ: 6.68 (s, 1H), 4.56 (s, 1H), 4.21-4.15 (m, 3H), 4.02 (s, 3H), 3.96-3.94 (m, 1H), 2.64-2.56 (m, 1H), 2.22-2.17 (m, 1H).

### Preparation 206: 4,6-dichloro-2-(2-fluoropropan-2-yl)pyrimidine

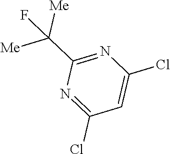 [SMILES: CC(C)(F)c1nc(Cl)cc(Cl)n1]

4,6-Dichloro-2-(2-fluoropropan-2-yl)pyrimidine was obtained a a yellow oil, 350 mg, 69%, from 2-(4,6-dichloropyrimidin-2-yl)propan-2-ol (Preparation 202), following an analogous procedure to that described in Preparation 176. ¹H NMR (500 MHz, CDCl₃) δ: 7.34 (s, 1H), 1.80 (s, 3H), 1.75 (s, 3H).

### Preparation 207: 4,6-dichloro-2-(3-fluorooxetan-3-yl)pyrimidine

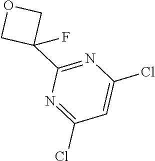 [SMILES: FC1(c2nc(Cl)cc(Cl)n2)COC1]

4,6-Dichloro-2-(3-fluorooxetan-3-yl)pyrimidine was prepared as a yellow oil (350 mg, 91%) from 3-(4,6-dichloropyrimidin-2-yl)oxetan-3-ol (Preparation 203), following an analogous procedure to that described in Preparation 176. ¹H NMR: (400 MHz, CDCl₃) δ: ppm 7.44 (s, 1H), 5.20-5.17 (m, 1H), 5.14-5.12 (m, 1H), 5.09-5.07 (m, 1H), 5.04-5.02 (m, 1H).

### Preparation 208: 4-chloro-2-(2-fluoropropan-2-yl)-6-methoxypyrimidine

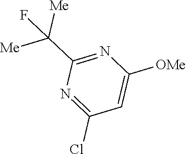 [SMILES: COc1cc(Cl)nc(C(C)(C)F)n1]

To a solution of 4,6-dichloro-2-(2-fluoropropan-2-yl)pyrimidine (Preparation 206, 350 mg, 1.67 mmol) in MeOH (5 mL) was added NaOMe (90.5 mg, 1.67 mmol) and the resulting mixture was stirred at 10° C. for 12 h. The mixture was concentrated under reduced pressure and the residue purified by chromatography on silica gel (PE/EtOAc=5/1) to give 4-chloro-2-(2-fluoropropan-2-yl)-6-methoxypyrimidine (280 mg, 82%) as a yellow oil. ¹H NMR (500 MHz, CDCl₃) δ: 6.59 (s, 1H), 3.93 (s, 3H), 1.69 (t, 6H).

### Preparation 209: 4-chloro-2-(3-fluorooxetan-3-yl)-6-methoxypyrimidine

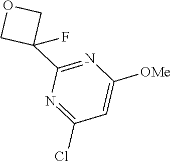 [SMILES: COc1cc(Cl)nc(C2(F)COC2)n1]

4-Chloro-2-(3-fluorooxetan-3-yl)-6-methoxypyrimidine was prepared as a white solid (260 mg, 76%) from 4,6-dichloro-2-(3-fluorooxetan-3-yl)pyrimidine (Preparation 207) using an analogous method to that described for Preparation 208. ¹H NMR (400 MHz, CDCl₃) δ: 6.75 (s, 1H), 5.20-5.13 (m, 2H), 5.07-4.99 (m, 2H), 4.04 (s, 3H).

### Preparation 210: 4-chloro-2-(3-fluorotetrahydrofuran-3-yl)-6-methoxypyrimidine

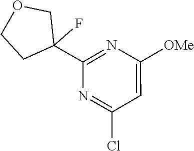 [SMILES: COc1cc(Cl)nc(C2(F)CCOC2)n1]

4-Chloro-2-(3-fluorotetrahydrofuran-3-yl)-6-methoxypyrimidine was prepared as a colourless oil (141 mg, 70%) from 3-(4-chloro-6-methoxypyrimidin-2-yl)tetrahydrofuran-3-ol (Preparation 205) using an analogous method to that described for Preparation 176. ¹H NMR (500 MHz, CDCl₃) δ: 6.71 (s, 1H), 4.32-4.15 (m, 4H), 4.02 (s, 3H), 2.74-2.69 (m, 1H), 2.67-2.53 (m, 1H).

### Preparation 211: 4-chloro-6-(1-ethoxyvinyl)-2-methylpyrimidine

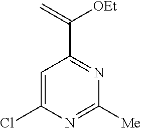

A solution of 4,6-dichloro-2-methylpyrimidine (5 g, 30.67 mmol) and tributyl(1-ethoxyvinyl)stannane (11.08 g, 30.67 mmol) in DMF (40 mL) was added Pd(PPh₃)₄ (1.77 g, 1.53 mmol) under N₂ and the mixture stirred at 120° C. for 12 h. The mixture was quenched with KF solution (20 mL) and extracted with DCM (40 mL×3). The combined organics were washed (brine), dried (Na₂SO₄) and concentrated under reduced pressure. The residue was purified by chromatography (PE/EtOAc=1/0 to 19/1) on silica gel to give 4-chloro-6-(1-ethoxyvinyl)-2-methylpyrimidine (2.9 g, 48%) as a colorless oil. ¹H NMR (500 MHz, CDCl₃) δ: 7.47 (s, 1H), 5.69 (d, 1H), 4.53 (d, J=2.5 Hz, 1H), 3.96 (q, 2H), 2.70 (s, 3H), 1.44 (t, 3H).

### Preparation 212: 1-(6-chloro-2-methylpyrimidin-4-yl)ethan-1-one

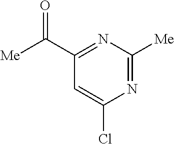 [SMILES: CC(=O)c1cc(Cl)nc(C)n1]

To a solution of 4-chloro-6-(1-ethoxyvinyl)-2-methylpyrimidine (Preparation 211, 2.9 g, 14.6 mmol) in acetone (15 mL) was added HCl (3 M, 14.60 mL) and the mixture stirred at 25° C. for 2 h. The reaction was extracted with EtOAc (20 mL×3). The combined organics were washed with brine (20 mL), dried (Na₂SO₄) and concentrated in vacuo. The residue was purified on silica gel column chromatography (PE/EtOAc=19/1) to give 1-(6-chloro-2-methylpyrimidin-4-yl)ethan-1-one (712 mg, 28%) as a colorless oil. ¹H NMR (500 MHz, CDCl₃) δ: 7.71 (s, 1H), 2.79 (s, 3H), 2.69 (s, 3H).

### Preparation 213: 4-chloro-6-(1,1-difluoroethyl)-2-methylpyrimidine

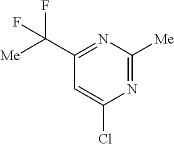 [SMILES: Cc1nc(Cl)cc(C(C)(F)F)n1]

4-Chloro-6-(1,1-difluoroethyl)-2-methylpyrimidine was prepared as a colourless oil (186 mg, 24%) from 1-(6-chloro-2-methylpyrimidin-4-yl)ethan-1-one (Preparation 212) using an analogous method to the described for Preparation 176. ¹H NMR (500 MHz, CDCl₃) δ: 7.46 (s, 1H), 2.76 (s, 3H), 1.97 (t, 3H).

### Preparation 214: Methyl 4-(benzyloxy)-6-methylpyrimidine-2-carboxylate

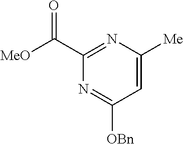 [SMILES: COC(=O)c1nc(C)cc(OCc2ccccc2)n1]

To a solution of 4-(benzyloxy)-2-chloro-6-methylpyrimidine (4.5 g, 19.17 mmol) and TEA (9.7 g, 95.87 mmol) in MeOH (50 mL) was added Pd(dppf)Cl₂ (1.40 g, 1.92 mmol) and the mixture was stirred at 80° C. for 16 h under CO (50 psi). The mixture was concentrated under reduced pressure to give a residue which was purified on silica gel column chromatography (PE/EtOAc=9/1 to 5/1) to give methyl 4-(benzyloxy)-6-methylpyrimidine-2-carboxylate (2.8 g, 56%) as a green oil. ¹H NMR (500 MHz, MeOH-d₄) δ: 7.28-7.53 (m, 5H), 6.94 (s, 1H), 5.50 (s, 2H), 4.00 (s, 3H), 2.50 (s, 3H).

### Preparation 215: 1-(4-(benzyloxy)-6-methylpyrimidin-2-yl)ethan-1-one

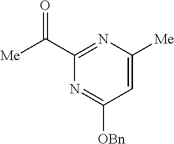 [SMILES: CC(=O)c1nc(C)cc(OCc2ccccc2)n1]

Under N₂, to a solution of methyl 4-(benzyloxy)-6-methylpyrimidine-2-carboxylate (Preparation 214, 2.6 g, 10.07 mmol) in THF (40 mL) was added CH₃MgBr (3 M, 4.03 mL) and the mixture was stirred for 2 h. The reaction mixture was quenched by the addition of water (20 mL) and extracted with EtOAc (40 mL×3). The combined organic layers were washed with brine (40 mL), dried (Na₂SO₄) and concentrated under reduced pressure to give a residue which was purified using silica gel column chromatography (PE/EtOAc=9/1 to 5/1) to give 1-(4-(benzyloxy)-6-methylpyrimidin-2-yl)ethan-1-one (312 mg, 13%) as a yellow oil. LCMS m/z=243.0 [M+H]⁺.

### Preparation 216: 4-(benzyloxy)-2-(1,1-difluoroethyl)-6-methylpyrimidine

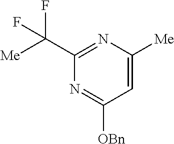 [SMILES: Cc1cc(OCc2ccccc2)nc(C(C)(F)F)n1]

4-(Benzyloxy)-2-(1,1-difluoroethyl)-6-methylpyrimidine was obtained (208 mg, 61.1% yield) as a yellow oil from 1-(4-(benzyloxy)-6-methylpyrimidin-2-yl)ethan-1-one (Preparation 215), following an analogous procedure to that described in Preparation 176. LCMS m/z=265.1 [M+H]⁺.

### Preparation 217:2-(1,1-difluoroethyl)-6-methylpyrimidin-4-ol

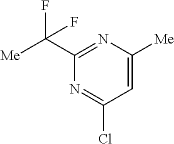 [SMILES: Cc1cc(Cl)nc(C(C)(F)F)n1]

A mixture of 4-(benzyloxy)-2-(1,1-difluoroethyl)-6-methylpyrimidine (Preparation 216, 208 mg, 0.8 mmol) and TFA (1 mL) was stirred at 100° C. for 12 h. The mixture was concentrated under reduced pressure to give a residue, which was purified on silica gel column chromatography (DCM/MeOH=19/1) to give 2-(1,1-difluoroethyl)-6-methylpyrimidin-4-ol (124 mg, 90%) as a yellow solid. ¹H NMR (500 MHz, MeOH-d₄) δ: 6.37 (s, 1H), 2.34 (s, 3H), 1.98 (t, 3H).

### Preparation 218: 4-chloro-2-(1,1-difluoroethyl)-6-methylpyrimidine

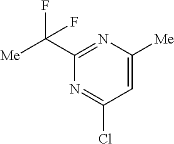 [SMILES: Cc1cc(Cl)nc(C(C)(F)F)n1]

A mixture of 2-(1,1-difluoroethyl)-6-methylpyrimidin-4-ol (Preparation 217, 124 mg, 0.7 mmol) and POCl₃ (1 mL) was stirred at 100° C. for 1 h. The reaction mixture was added to ice water (5 mL) dropwise and extracted with EtOAc (10 mL×3). The combined organics were washed with brine (15 mL), dried (Na₂SO₄) and evaporated to dryness in vacuo to give 4-chloro-2-(1,1-difluoroethyl)-6-methylpyrimidine (86 mg, 63%) as a brown oil. LCMS m/z=193.1 [M+H]⁺.

### Preparation 219: 2-chloro-4-(1,1-difluoroethyl)-6-methoxypyrimidine and 4-chloro-6-(1,1-difluoroethyl)-2-methoxypyrimidine

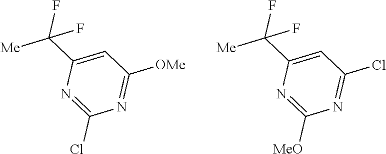 [SMILES: COc1cc(C(C)(F)F)nc(Cl)n1 ; COc1nc(Cl)cc(C(C)(F)F)n1]

To a solution of 2,4-dichloro-6-(1,1-difluoroethyl)pyrimidine (380 mg, 1.78 mmol) in MeOH (5 mL) was added MeONa (106.0 mg, 1.96 mmol) and the resulting mixture was stirred at 35° C. for 16 h. The mixture was concentrated and the residue was purified by chromatography on silica gel (PE/EtOAc=5/1) to give a mixture of 2-chloro-4-(1,1-difluoroethyl)-6-methoxypyrimidine and 4-chloro-6-(1,1-difluoroethyl)-2-methoxypyrimidine (240 mg, 64.5% yield) as a yellow oil. ¹H NMR: (400 MHz, CDCl₃) δ: ppm 6.94 (s, 1H), 4.05 (s, 3H), 1.95 (t, J=18.8 Hz, 3H).

### Preparation 220:2-chloro-4-(1,1-difluoroethyl)-6-isopropoxypyrimidine

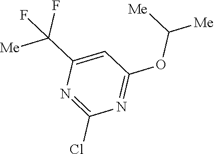 [SMILES: CC(C)Oc1cc(C(C)(F)F)nc(Cl)n1]

A solution of Na (10.8 mg, 0.469 mmol) in IPA (2 mL) was stirred at 50° C. for 2 h. To this was added 2,4-dichloro-6-(1,1-difluoroethyl)pyrimidine (100 mg, 0.469 mmol) and the mixture stirred at 50° C. for 12 h. The mixture was concentrated under reduced pressure and the residue purified by chromatography on silica gel (PE/EtOAc=10/1) to give 2-chloro-4-(1,1-difluoroethyl)-6-isopropoxypyrimidine as a white solid (45.2 mg, 41%). ¹H NMR (500 MHz, CDCl₃) δ: 6.94 (s, 1H), 4.05 (s, 3H), 1.95 (t, 3H).

### Preparation 221: 2,4-dichloro-6-(2-fluoropropan-2-yl)pyrimidine

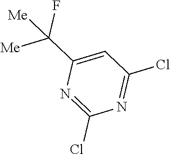 [SMILES: CC(C)(F)c1cc(Cl)nc(Cl)n1]

To a solution of 2-(2,6-dichloropyrimidin-4-yl)propan-2-ol (210 mg, 1.01 mmol) in DCM (3 mL) was added DAST (327 mg, 2.03 mmol) at 0° C. and the resulting mixture was stirred at 25° C. for 12 h. The reaction mixture was poured into ice-water (10 mL) slowly and extracted with DCM (3×10 mL). The combined organics were washed with brine (2×10 mL), dried (Na₂SO₄) and concentrated under reduced pressure. The residue was purified by chromatography (SiO₂, 25% EtOAc/PE) to give 4-dichloro-6-(2-fluoropropan-2-yl)pyrimidine as a yellow solid (127.5 mg, 60%). ¹H NMR (400 MHz, CDCl₃) δ: 7.54 (d, 1H), 1.72 (s, 3H), 1.66 (s, 3H).

### Preparation 222: 2-chloro-4-(2-fluoropropan-2-yl)pyrimidine

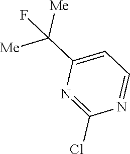 [SMILES: CC(C)(F)c1ccnc(Cl)n1]

The title compound was prepared as a yellow oil (140 mg, 33%) from 2-(2-chloropyrimidin-4-yl)propan-2-ol using an analogous method to that described for Preparation 221. ¹H NMR (500 MHz, CDCl₃) δ: 8.67-8.61 (m, 1H), 7.54-7.48 (m, 1H), 1.72 (s, 3H), 1.67 (s, 3H).

### Preparation 223: 2-chloro-4-(2-fluoropropan-2-yl)-6-methoxypyrimidine

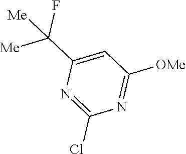 [SMILES: COc1cc(C(C)(C)F)nc(Cl)n1]

2-Chloro-4-(2-fluoropropan-2-yl)-6-methoxypyrimidine was prepared as a colourless oil (50 mg, 40%) from 2,4-dichloro-6-(2-fluoropropan-2-yl)pyrimidine (Preparation 221) using an analogous method to that described for Preparation 208. ¹H NMR (400 MHz, CDCl₃) δ: 6.85 (d, 1H), 4.01 (s, 3H), 1.68 (s, 3H), 1.63 (s, 3H).

### Preparation 224: 2-chloro-4-(1,1-difluoroethyl)-6-(2-methoxyethoxy)pyrimidine and 4-chloro-6-(1,1-difluoroethyl)-2-(2-methoxyethoxy)pyrimidine

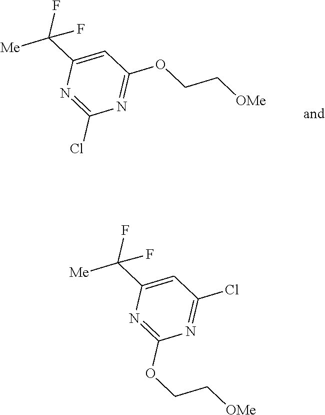 [SMILES: COCCOc1cc(C(C)(F)F)nc(Cl)n1 ; COCCOc1nc(Cl)cc(C(C)(F)F)n1]

To a solution of 2-methoxyethan-1-ol (35.7 mg, 0.469 mmol) in THF (3 mL) was added NaH (28.2 mg, 0.704 mmol, 60% purity) and the mixture stirred 30 mins. To this was added 2,4-dichloro-6-(1,1-difluoroethyl)pyrimidine (100 mg, 0.469 mmol) in THF (0.5 mL) and the resulting mixture stirred at 25° C. for 12 h. The mixture was evaporated to dryness in vacuo and was purified by chromatography (SiO₂, 20% EtOAc/PE) to give a mixture of 2-chloro-4-(1,1-difluoroethyl)-6-(2-methoxyethoxy)pyrimidine and 4-chloro-6-(1,1-difluoroethyl)-2-(2-methoxyethoxy)pyrimidine as a colourless oil (71.2 mg, crude) which was used without further purification. ¹H NMR (400 MHz, CDCl₃) δ: 7.54-7.52 (m, 1H), 4.86-4.83 (m, 2H), 4.01-3.99 (m, 2H), 3.69 (s, 3H), 2.24-2.15 (m, 3H).

### Preparation 225: 4-(6-chloro-1-trityl-1H-pyrazolo[4,3-c]pyridin-3-yl)-1-methylpiperazin-2-one

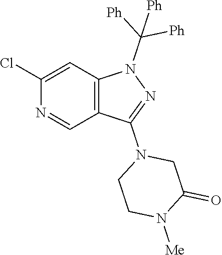 [SMILES: CN1CCN(c2nn(C(c3ccccc3)(c3ccccc3)c3ccccc3)c3cc(Cl)ncc23)CC1=O]

To a mixture of 6-chloro-3-iodo-1-trityl-1H-pyrazolo[4,3-c]pyridine (Preparation 14, 3.0 g, 5.75 mmol) and 1-methylpiperazin-2-one (984.4 mg, 8.62 mmol) in DMSO (30 mL) was added CuI (219 mg, 1.15 mmol), K₂CO₃ (1.6 g, 11.5 mmol) and L-proline (264.8 mg, 2.30 mmol) and the reaction mixture stirred at 100° C. under N₂ for 12 h. The mixture was poured into H₂O (50 mL) and extracted with EtOAc (60 mL×3). The combined organic phase was washed with brine (2×30 mL), dried (Na₂SO₄) and evaporated to dryness in vacuo. The residue was purified by chromatography (SiO₂, 5-50% EtOAc/PE) to give 4-(6-chloro-1-trityl-1H-pyrazolo[4,3-c]pyridin-3-yl)-1-methylpiperazin-2-one as a yellow solid (622 mg, 21%). ¹H NMR (500 MHz, MeOH-d₄) δ: 8.86 (s, 1H), 7.33-7.31 (m, 9H), 7.24-7.22 (m, 6H), 6.01 (s, 1H), 4.00 (s, 2H), 3.75-3.73 (m, 2H), 3.51-3.48 (m, 2H), 2.97 (s, 3H).

### Preparation 226: 3-(azetidin-1-yl)-6-chloro-1-trityl-1H-pyrazolo[4,3-c]pyridine

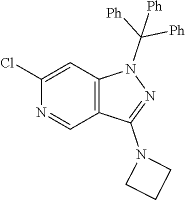 [SMILES: Clc1cc2c(cn1)c(N1CCC1)nn2C(c1ccccc1)(c1ccccc1)c1ccccc1]

3-(Azetidin-1-yl)-6-chloro-1-trityl-1H-pyrazolo[4,3-c]pyridine was prepared as a yellow solid (483 mg, 56%) from 6-chloro-3-iodo-1-trityl-1H-pyrazolo[4,3-c]pyridine (Preparation 14) and azetidine using an analogous method to that described for Preparation 225. ¹H NMR (500 MHz, CDCl₃) δ: ppm: 8.51 (s, 1H), 7.31-7.28 (m, 10H), 7.25-7.22 (m, 5H), 5.80 (s, 1H), 4.13-4.10 (m, 4H), 2.48-2.42 (m, 2H).

### Preparation 227: 4-(6-chloro-1H-pyrazolo[4,3-c]pyridin-3-yl)-1-methylpiperazin-2-one

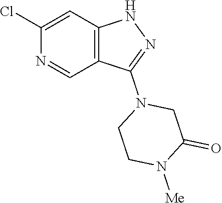 [SMILES: CN1CCN(c2n[nH]c3cc(Cl)ncc23)CC1=O]

To a solution of 4-(6-chloro-1-trityl-1H-pyrazolo[4,3-c]pyridin-3-yl)-1-methylpiperazin-2-one (Preparation 225, 622 mg, 1.22 mmol) in DCM (5 mL) was added TFA (2.9 g, 26.12 mmol) and Et₃SiH (570 mg, 4.90 mmol) and the resulting mixture stirred at 25° C. for 2 h. The mixture was concentrated and purified by prep-HPLC-H (25-45% MeCN) to give 4-(6-chloro-1H-pyrazolo[4,3-c]pyridin-3-yl)-1-methylpiperazin-2-one as a yellow solid (223 mg, 69%). ¹H NMR (500 MHz, CDCl₃) δ: 8.98 (s, 1H), 7.35 (s, 1H), 4.28 (s, 2H), 3.83-3.81 (m, 2H), 3.61-3.59 (m, 2H), 3.10 (s, 3H).

### Preparation 228: 3-(azetidin-1-yl)-6-chloro-1H-pyrazolo[4,3-c]pyridine

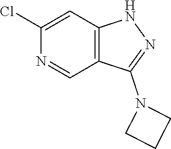 [SMILES: Clc1cc2[nH]nc(N3CCC3)c2cn1]

3-(Azetidin-1-yl)-6-chloro-1H-pyrazolo[4,3-c]pyridine was prepared as a yellow solid (110 mg, 53%) from 3-(azetidin-1-yl)-6-chloro-1-trityl-1H-pyrazolo[4,3-c]pyridine (Preparation 226) using an analogous method to that described for Preparation 227. ¹H NMR (500 MHz, CDCl₃) δ: 8.64 (s, 1H), 7.21 (s, 1H), 4.27-4.24 (m, 4H), 2.58-2.52 (m, 2H).

### Preparation 229: 6-chloro-3-(3,4-dimethylpiperazin-1-yl)-1-(tetrahydro-2H-pyran-2-yl)-1H-pyrazolo[4,3-c]pyridine

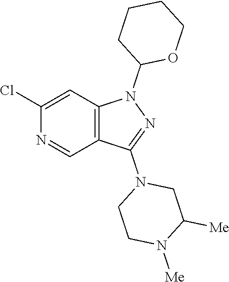 [SMILES: CC1CN(c2nn(C3CCCCO3)c3cc(Cl)ncc23)CCN1C]

To a solution of 6-chloro-3-iodo-1-(tetrahydro-2H-pyran-2-yl)-1H-pyrazolo[4,3-c]pyridine (400 mg, 1.10 mmol) in DMSO (1 mL) under N₂ was added 1,2-dimethylpiperazine (138.2 mg, 1.21 mmol), CuI (41.91 mg, 0.220 mmol), L-proline (38 mg, 0.33 mmol) and K₂CO₃ (304 mg, 2.20 mmol) and the mixture stirred at 100° C. for 3 h. The mixture was quenched with H₂O (30 mL) and extracted with EtOAc (2×30 mL). The combined organics were washed with brine (2×50 mL), dried (Na₂SO₄) and evaporated to dryness in vacuo. The residue was purified by chromatography (SiO2, 16-50% EtOAc/PE) to give 6-chloro-3-(3,4-dimethylpiperazin-1-yl)-1-(tetrahydro-2H-pyran-2-yl)-1H-pyrazolo[4,3-c]pyridine as a yellow oil (150 mg, 39%). ¹H NMR (500 MHz, CDCl₃) δ: 8.76 (s, 1H), 7.34 (s, 1H), 5.46-5.43 (m, 1H), 4.06-4.03 (m, 1H), 3.90-3.67 (m, 3H), 3.35-2.83 (m, 3H), 2.56-2.43 (m, 2H), 2.39 (s, 3H), 2.14-2.07 (m, 1H), 2.00-1.96 (m, 1H), 1.77-1.63 (m, 4H), 1.19 (s, 3H).

### Preparation 230-248

The title compounds were prepared from 6-chloro-3-iodo-1-(tetrahydro-2H-pyran-2-yl)-1H-pyrazolo[4,3-c]pyridine and the appropriate amine using an analogous method to that described for Preparation 229.

| Preparation | |
|---|---|
| No | Name/Structure/R¹R²NH/Data |
| 230 | 6-chloro-3-(3-methoxypiperidin-1-yl)-1-(tetrahydro-2H-pyran-2-yl)-1H- |
| | pyrazolo[4,3-c]pyridine |
| | 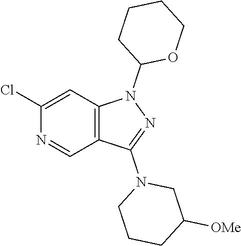 [SMILES: COC1CCCN(c2nn(C3CCCCO3)c3cc(Cl)ncc23)C1] |
| | Amine: 3-methoxypiperidine |
| | Yellow solid (259 mg, 67%); LCMS m/z = 351.1 [M + H]⁺. |
| 231 | 6-chloro-3-(3-methoxypyrrolidin-1-yl)-1-(tetrahydro-2H-pyran-2-yl)-1H- |
| | pyrazolo[4,3-c]pyridine |
| | 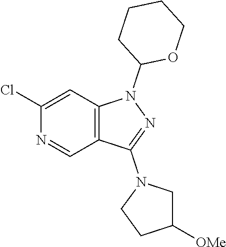 [SMILES: COC1CCN(c2nn(C3CCCCO3)c3cc(Cl)ncc23)C1] |
| | Amine: 3-mthoxypyrrolidine hydrochloride |
| | Yellow oil (190 mg, 41%); ¹H NMR (500 MHz, CDCl₃) δ: 8.75 (s, 1H), |
| | 7.28 (d, 1H), 5.43-5.40 (m, 1H), 4.13-4.04 (m, 2H), 3.75-3.70 (m, 5H), 3.38 |
| | (s, 3H), 2.20-2.19 (m, 1H), 2.14-2.13 (m, 3H), 2.12-2.11 (m, 1H), 1.72-1.70 |
| | (m, 3H). |
| 232 | 1-(6-chloro-1-(tetrahydro-2H-pyran-2-yl)-1H-pyrazolo[4,3-c]pyridin-3- |
| | yl)pyrrolidin-3-ol |
| | 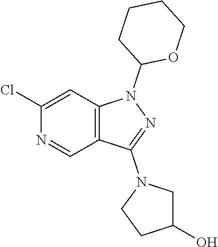 [SMILES: OC1CCN(c2nn(C3CCCCO3)c3cc(Cl)ncc23)C1] |
| | Amine: 3-hydroxypyrrolidine |
| | Yellow solid (242 mg, 68%); ¹H NMR (500 MHz, CDCl₃) δ: 8.75 (s, 1H), |
| | 7.29 (s, 1H), 5.44-5.40 (m, 1H), 4.66-4.65 (m, 1H), 4.04-3.83 (m, 1H), |
| | 3.81-3.65 (m, 5H), 2.44-2.42 (m, 1H), 2.21-2.19 (m, 3H), 2.12-2.10 (m, |
| | 1H), 1.73-1.68 (m, 4H). |
| 233 | 1-(6-chloro-1-(tetrahydro-2H-pyran-2-yl)-1H-pyrazolo[4,3-c]pyridin-3-yl)- |
| | 3-methylpyrrolidin-3-ol |
| | 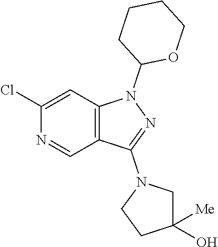 [SMILES: CC1(O)CCN(c2nn(C3CCCCO3)c3cc(Cl)ncc23)C1] |
| | Amine: 3-methylpyrrolidin-3-ol |
| | Yellow oil (261 mg, 56%); ¹H NMR (400 MHz, MeOH-d₄) δ: 8.76 (s, 1H), |
| | 7.50 (s, 1H), 5.58-5.55 (m, 1H), 3.99-3.95 (m, 1H), 3.85-3.83 (m, 1H), |
| | 3.79-3.74 (m, 2H), 3.62-3.56 (m, 2H), 2.43-2.37 (m, 1H), 2.09-2.05 (m, |
| | 3H), 1.94-1.90 (m, 1H), 1.76-1.63 (m, 3H), 1.49 (s, 3H) |
| 234 | 6-chloro-3-(3-fluoroazetidin-1-yl)-1-(tetrahydro-2H-pyran-2-yl)-1H- |
| | pyrazolo[4,3-c]pyridine |
| | 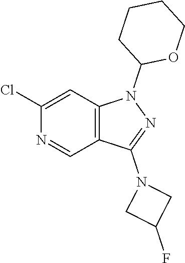 [SMILES: FC1CN(c2nn(C3CCCCO3)c3cc(Cl)ncc23)C1] |
| | Amine: 3-fluoroazetidine |
| | White solid (219 mg, 73%); ¹H NMR (400 MHz, CDCl₃) δ: 8.54 (s, 1H), |
| | 7.33 (s, 1H), 5.58-5.38 (m, 2H), 4.58-4.43 (m, 2H), 4.39-4.26 (m, 2H), |
| | 4.13-3.98 (m, 1H), 3.80-3.65 (m, 1H), 2.47-2.34 (m, 1H), 2.13-1.93 (m, |
| | 2H), 1.77-1.60 (m, 3H). |
| 235 | 6-chloro-3-(2-methylazetidin-1-yl)-1-(tetrahydro-2H-pyran-2-yl)-1H- |
| | pyrazolo[4,3-c]pyridine |
| | 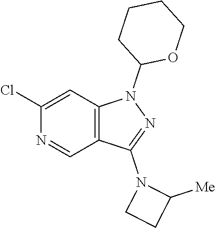 [SMILES: CC1CCN1c1nn(C2CCCCO2)c2cc(Cl)ncc12] |
| | Amine: 2-methylazetidine hydrochloride |
| | White solid (72 mg, 43%); ¹H NMR (500 MHz, MeOH-d₄) δ: 8.60 (s, 1H), |
| | 7.56 (d, 1H), 5.62-5.59 (m, 1H), 4.53-4.51 (m, 1H), 4.26-4.23 (m, 1H), |
| | 4.03-3.99 (m, 2H), 3.80-3.78 (m, 1H), 2.55-2.53 (m, 1H), 2.45-2.43 (m, |
| | 1H), 2.30-2.08 (m, 2H), 1.98-1.90 (m, 1H), 1.79-1.64 (m, 3H), 1.55-1.51 |
| | (m, 3H). |
| 236 | 6-chloro-3-(3-methoxyazetidin-1-yl)-1-(tetrahydro-2H-pyran-2-yl)-1H- |
| | pyrazolo[4,3-c]pyridine |
| | 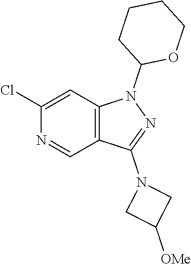 [SMILES: COC1CN(c2nn(C3CCCCO3)c3cc(Cl)ncc23)C1] |
| | Amine: 3-methoxyazetidine |
| | White solid (320 mg, 72%); LCMS m/z = 323.0 [M + H]⁺. |
| 237 | 1-(6-chloro-1-(tetrahydro-2H-pyran-2-yl)-1H-pyrazolo[4,3-c]pyridin-3- |
| | yl)azetidin-3-ol |
| | 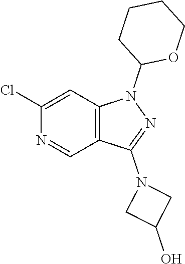 [SMILES: OC1CN(c2nn(C3CCCCO3)c3cc(Cl)ncc23)C1] |
| | Amine: 3-hydroxyazetidine |
| | White solid (200 mg, 47%); LCMS m/z = 309.1 [M + H]⁺. |
| 238 | 1-(6-chloro-1-(tetrahydro-2H-pyran-2-yl)-1H-pyrazolo[4,3-c]pyridin-3-yl)- |
| | 3-methylazetidin-3-ol |
| | 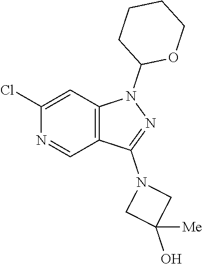 [SMILES: CC1(O)CN(c2nn(C3CCCCO3)c3cc(Cl)ncc23)C1] |
| | Amine: 3-hydroxyazetidine |
| | Colourless oil (330 mg, 74%); LCMS m/z = 323.3 [M + H]⁺. |
| 239 | 6-chloro-3-(3-methoxy-3-methylazetidin-1-yl)-1-(tetrahydro-2H-pyran-2- |
| | yl)-1H-pyrazolo[4,3-c]pyridine |
| | 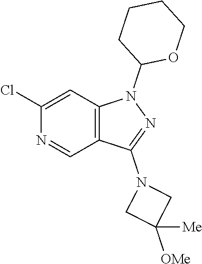 [SMILES: COC1(C)CN(c2nn(C3CCCCO3)c3cc(Cl)ncc23)C1] |
| | Amine: 3-methoxy-3-methylazetidine |
| | Colourless oil (380 mg, 82%); ¹H NMR (500 MHz, CDCl₃) δ: 8.49 (s, 1H), |
| | 7.25 (s, 1H), 5.38-5.34 (m, 1H), 4.13-4.08 (m, 2H), 4.03-3.98 (m, 1H), |
| | 3.96-3.91 (m, 2H), 3.66-3.63 (m, 1H), 3.23 (s, 3H), 2.34-2.30 (m, 1H), |
| | 2.05-2.03 (m, 1H), 1.93-1.88 (m, 1H), 1.67-1.64 (m, 1H), 1.62 (s, 3H), |
| | 1.21-1.17 (m, 2H). |
| 240 | 6-chloro-1-(tetrahydro-2H-pyran-2-yl)-3-(3-(trifluoromethoxy)azetidin-1- |
| | yl)-1H-pyrazolo[4,3-c]pyridine |
| | 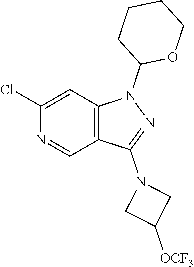 [SMILES: FC(F)(F)OC1CN(c2nn(C3CCCCO3)c3cc(Cl)ncc23)C1] |
| | Amine: 3-trifluoromethoxyazetidine |
| | Yellow oil (53 mg, 26%); ¹H NMR (400 MHz, MeOH-d₄) δ: 8.63 (s, 1H), |
| | 7.58 (s, 1H), 5.63-5.60 (m, 1H), 4.60-4.56 (m, 2H), 4.28-4.25 (m, 2H), |
| | 4.11-4.08 (m, 2H), 3.97-3.77 (m, 1H), 2.40-2.35 (m, 1H), 2.08-2.05 (m, |
| | 1H), 1.97-1.93 (m, 1H), 1.80-1.77 (m, 1H), 1.68-1.64 (m, 2H). |
| 241 | 1-(6-chloro-1-(tetrahydro-2H-pyran-2-yl)-1H-pyrazolo[4,3-c]pyridin-3-yl)- |
| | N,N-dimethylazetidin-3-amine |
| | 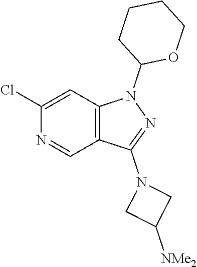 [SMILES: CN(C)C1CN(c2nn(C3CCCCO3)c3cc(Cl)ncc23)C1] |
| | Amine: 3-dimethylaminoazetidine hydrochloride |
| | White solid (300 mg, 65%); ¹H NMR (500 MHz, CDCl₃) δ: 8.53 (s, 1H), |
| | 7.29 (s, 1H), 5.41-5.38 (m, 1H), 4.25-4.23 (m, 2H), 4.05-4.02 (m, 3H), |
| | 3.68-3.67 (m, 1H), 3.38-3.35 (m, 1H), 2.40-2.33 (m, 1H), 2.23 (s, 6H), |
| | 2.08-2.05 (m, 1H), 1.95-1.94 (m, 1H), 1.70-1.67 (m, 2H), 1.64-1.59 (m, |
| | 1H). |
| 242 | 6-(6-chloro-1-(tetrahydro-2H-pyran-2-yl)-1H-pyrazolo[4,3-c]pyridin-3-yl)- |
| | 2-oxa-6-azaspiro[3.3]heptane |
| | 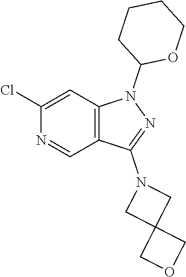 [SMILES: Clc1cc2c(cn1)c(N1CC3(COC3)C1)nn2C1CCCCO1] |
| | Amine: 2-oxa-6-azaspiro[3.3]heptane |
| | Yellow solid (450 mg, 70%); ¹H NMR (400 MHz, CDCl₃) δ: 8.56 (s, 1H), |
| | 7.31 (s, 1H), 5.43-5.40 (m, 1H), 4.86 (s, 4H), 4.39-4.32 (m, 4H), 4.11-4.05 |
| | (m, 1H), 3.73-3.67 (m, 1H), 2.10-2.09 (m, 1H), 1.98-1.95 (m, 1H), 1.74- |
| | 1.68 (m, 4H). |
| 243 | tert-butyl (1-(6-chloro-1-(tetrahydro-2H-pyran-2-yl)-1H-pyrazolo[4,3- |
| | c]pyridin-3-yl)pyrrolidin-3-yl)carbamate |
| | 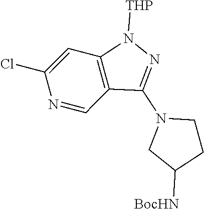 |
| | 6-chloro-3-iodo-1-(tetrahydro-2H-pyran-2-yl)-1H-pyrazolo[4,3-c]pyridine |
| | Amine: tert-butyl pyrrolidin-3-ylcarbamate |
| | White solid (1.7 g, 73%); LCMS m/z = 422.1 [M + H]⁺ |
| 244 | 1-(6-chloro-1-(tetrahydro-2H-pyran-2-yl)-1H-pyrazolo[4,3-c]pyridin-3-yl)- |
| | 6-oxa-1-azaspiro[3.3]heptane |
| | 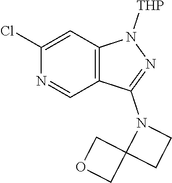 |
| | Amine: 6-oxa-1-azaspiro[3.3]heptane |
| | Yellow solid (105 mg, 38%); ¹H NMR (500 MHz, CDCl₃) δ: 8.80 (s, 1H), |
| | 7.35 (s, 1H), 5.50-5.46 (m, 1H), 5.45-5.39 (m, 2H), 4.13-4.09 (m, 4H), |
| | 3.72-3.69 (m, 1H), 2.75-2.72 (m, 2H), 2.46-2.41 (m, 1H), 2.12-2.09 (m, |
| | 1H), 2.02-1.99 (m, 1H), 1.77-1.59 (m, 4H). |
| 245 | 1-(6-chloro-1-(tetrahydro-2H-pyran-2-yl)-1H-pyrazolo[4,3-c]pyridin-3-yl)- |
| | N,N,3-trimethylpyrrolidin-3-amine |
| | 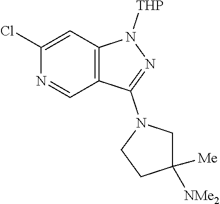 |
| | Amine: N,N,3-trimethylpyrrolidin-3-amine |
| | White solid (123 mg, 41%); ¹H NMR (400 MHz, MeOH-d₄) δ: 8.78 (s, 1H), |
| | 7.52 (s, 1H), 5.59-5.56 (m, 1H), 4.02-3.99 (m, 1H), 3.80-3.76 (m, 3H), |
| | 3.66-3.54 (m, 2H), 2.43-2.40 (m, 1H), 2.37 (s, 6H), 2.13-2.04 (m, 3H), |
| | 1.94-1.91 (m, 1H), 1.76-1.63 (m, 3H), 1.20 (s, 3H). |
| 246 | tert-butyl 6-(6-chloro-1-(tetrahydro-2H-pyran-2-yl)-1H-pyrazolo[4,3- |
| | c]pyridin-3-yl)-3,6-diazabicyclo[3.2.0]heptane-3-carboxylate |
| | 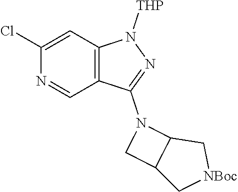 |
| | Amine: tert-butyl 3,6-diazabicyclo[3.2.0]heptane-3-carboxylate |
| | White solid (197 mg, 55%); ¹H NMR (500 MHz, CDCl₃) δ: 8.50 (s, 1H), |
| | 7.31 (s, 1H), 5.43-5.40 (m, 1H), 4.95-4.91 (m, 1H), 4.32-4.29 (m, 1H), |
| | 4.05-3.97 (m, 4H), 3.72-3.69 (m, 1H), 3.35-3.22 (m, 4H), 2.45-2.25 (m, |
| | 1H), 2.15-2.05 (m, 1H), 1.99-1.85 (m, 1H), 1.71-1.68 (m, 2H), 1.49-1.47 |
| | (m, 9H). |
| 247 | tert-butyl 5-(6-chloro-1-(tetrahydro-2H-pyran-2-yl)-1H-indazol-3- |
| | yl)hexahydropyrrolo[3,4-b]pyrrole-1(2H)-carboxylate |
| | 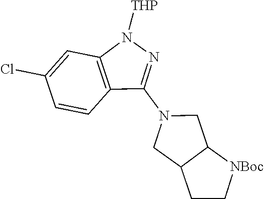 |
| | Amine: tert-butyl hexahydropyrrolo[3,4-b]pyrrole-1(2H)-carboxylate |
| | Yellow oil (58 mg, 31%); ¹H NMR (400 MHz, CDCl₃) δ: 8.77-8.73 (m, |
| | 1H), 7.32-7.29 (m, 1H), 5.44-5.41 (m, 1H), 4.50-4.39 (m, 1H), 3.86-3.70 |
| | (m, 1H), 3.52-3.45 (m, 8H), 3.08 (br s, 1H), 2.43 (br s, 1H), 2.12-2.07 (m, |
| | 2H), 1.99-1.89 (m, 2H), 1.72-1.51 (m, 2H), 1.47 (s, 9H). |
| 248 | tert-butyl 5-(6-chloro-1-(tetrahydro-2H-pyran-2-yl)-1H-pyrazolo[4,3- |
| | c]pyridin-3-yl)hexahydropyrrolo[3,4-c]pyrrole-2(1H)-carboxylate |
| | 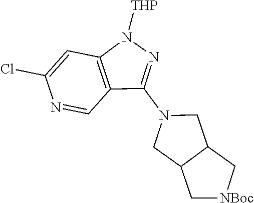 |
| | Amine: tert-butyl hexahydropyrrolo[3,4-c]pyrrole-2(1H)-carboxylate |
| | White solid (173 mg, 47%); LCMS m/z = 448.1 [M + H]⁺. |

### Preparation 249: 1-(6-chloro-1-(tetrahydro-2H-pyran-2-yl)-1H-pyrazolo[4,3-c]pyridin-3-yl)-3-methylpyrrolidine-3-carbonitrile

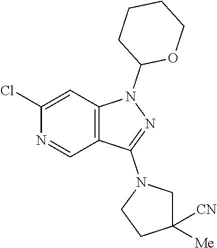 [SMILES: CC1(C#N)CCN(c2nn(C3CCCCO3)c3cc(Cl)ncc23)C1]

To a solution of 6-chloro-3-iodo-1-(tetrahydro-2H-pyran-2-yl)-1H-pyrazolo[4,3-c]pyridine (400 mg, 1.10 mmol) and 3-methylpyrrolidine-3-carbonitrile (193.6 mg, 1.32 mmol) in dioxane (10 mL) at 25° C. was added Xantphos Pd G3 (113.7 mg, 0.11 mmol) and Cs₂CO₃ (1.08 g, 3.30 mmol) and the mixture stirred at 110° C. for 16 h under N₂. The mixture was concentrated and purified by column chromatography (SiO₂, 16-50% EtOAc/PE) to give 1-(6-chloro-1-(tetrahydro-2H-pyran-2-yl)-1H-pyrazolo[4,3-c]pyridin-3-yl)-3-methylpyrrolidine-3-carbonitrile as a pale yellow oil (320 mg, 84%). LCMS m/z=346.0 [M+H]⁺; ¹H NMR (400 MHz, CDCl₃) δ: 8.72 (s, 1H), 7.31 (d, 1H), 5.44-5.41 (m, 1H), 4.09-3.85 (m, 4H), 3.71-3.69 (m, 1H), 3.58-3.53 (m, 1H), 2.58-2.55 (m, 1H), 2.44-2.36 (m, 1H), 2.15-2.09 (m, 2H), 1.99-1.95 (m, 1H), 1.76-1.66 (m, 3H), 1.61 (s, 3H).

### Preparation 250-257

The title compounds were prepared from 6-chloro-3-iodo-1-(tetrahydro-2H-pyran-2-yl)-1H-pyrazolo[4,3-c]pyridine and the appropriate amine using an analogous method to that described for Preparation 249. Alternative catalyst systems noted in the table.

| Preparation | |
|---|---|
| No | Name/Structure/R¹R²NH/Data |
| 250^{A} | 6-chloro-3-(3,3-difluoroazetidin-1-yl)-1-(tetrahydro-2H-pyran-2-yl)-1H- |
| | pyrazolo[4,3-c]pyridine |
| | 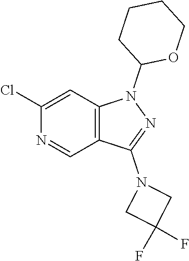 [SMILES: FC1(F)CN(c2nn(C3CCCCO3)c3cc(Cl)ncc23)C1] |
| | Amine: 3,3-difluoroazetidine |
| | Yellow solid (45 mg, 25%); ¹H NMR (500 MHz, CDCl₃) δ: 8.55 (s, 1H), |
| | 7.36 (s, 1H), 5.46-5.43 (m, 1H), 4.61-4.49 (m, 4H), 4.08-4.02 (m, 1H), |
| | 3.74-3.68 (m, 1H), 2.45-2.35 (m, 1H), 2.14-2.06 (m, 1H), 2.02-1.94 (m, |
| | 1H), 1.79-1.67 (m, 3H). |
| 251 | 1-(6-chloro-1-(tetrahydro-2H-pyran-2-yl)-1H-pyrazolo[4,3-c]pyridin-3-yl)- |
| | 3-fluoroazetidine-3-carbonitrile |
| | 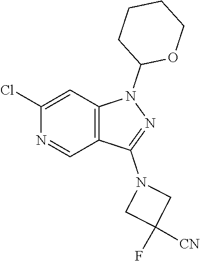 [SMILES: N#CC1(F)CN(c2nn(C3CCCCO3)c3cc(Cl)ncc23)C1] |
| | Amine: 3-fluoroazetidine-3-carbonitrile |
| | White solid (118 mg, 32%); ¹H NMR (500 MHz, CDCl₃) δ: 8.54 (s, 1H), |
| | 7.37 (s, 1H), 5.47-5.44 (m, 1H), 4.74-4.70 (m, 2H), 4.61-4.54 (m, 2H), |
| | 4.07-4.04 (m, 1H), 3.72-3.71 (m, 1H), 2.12-2.11 (m, 1H), 2.00-1.99 (m, |
| | 1H), 1.75-1.64 (m, 4H). |
| 252^{B} | tert-butyl 5-(6-chloro-1-(tetrahydro-2H-pyran-2-yl)-1H-pyrazolo[4,3- |
| | c]pyridin-3-yl)-2,5-diazabicyclo[2.2.2]octane-2-carboxylate |
| | 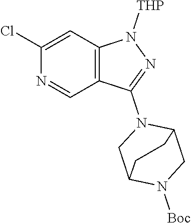 |
| | Amine: tert-butyl 2,5-diazabicyclo[2.2.2]octane-2-carboxylate |
| | White solid (181 mg, 74%); LCMS m/z = 448.1 [M + H]⁺. |
| 253^{B} | tert-butyl 3-(6-chloro-1-(tetrahydro-2H-pyran-2-yl)-1H-pyrazolo[4,3- |
| | c]pyridin-3-yl)-3,6-diazabicyclo[3.1.1]heptane-6-carboxylate |
| | 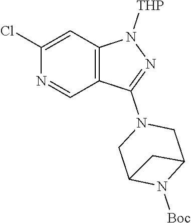 |
| | Amine: tert-butyl 3,6-diazabicyclo[3.1.1]heptane-6-carboxylate |
| | Yellow solid (120 mg, 50%); LCMS m/z = 434.1 [M + H]⁺. |
| 254^{B} | tert-butyl 3-(6-chloro-1-(tetrahydro-2H-pyran-2-yl)-1H-pyrazolo[4,3- |
| | c]pyridin-3-yl)-3,8-diazabicyclo[3.2.1]octane-8-carboxylate |
| | 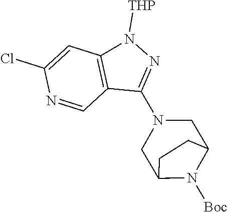 |
| | Amine: tert-butyl 3,8-diazabicyclo[3.2.1]octane-8-carboxylate |
| | Yellow oil (523 mg, 85%); ¹H NMR (400 MHz, CDCl₃) δ: 8.75 (s, 1H), |
| | 7.32 (s, 1H), 5.44-5.41 (m, 1H), 4.42-4.34 (m, 2H), 4.05-4.02 (m, 1H), |
| | 3.73-3.66 (m, 3H), 3.32-3.31 (m, 2H), 2.43-2.11 (m, 1H), 2.10-1.92 (m, |
| | 6H), 1.74-1.63 (m, 3H), 1.48 (s, 9H). |
| 255^{B} | tert-butyl 8-(6-chloro-1-(tetrahydro-2H-pyran-2-yl)-1H-pyrazolo[4,3- |
| | c]pyridin-3-yl)-3,8-diazabicyclo[3.2.1]octane-3-carboxylate |
| | 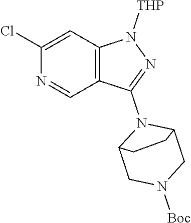 |
| | Amine: tert-butyl 3,8-diazabicyclo[3.2.1]octane-3-carboxylate |
| | Yellow solid (201 mg, 81%); ¹H NMR (500 MHz, CDCl₃) δ: 8.72 (s, 1H), |
| | 7.33 (s, 1H), 5.46-5.44 (m, 1H), 4.42-4.35 (m, 2H), 4.03-4.00 (m, 1H), |
| | 3.86-3.85 (m, 1H), 3.83-3.68 (m, 2H), 3.33-3.30 (m, 2H), 2.40-2.38 (m, |
| | 1H), 2.11-2.09 (m, 2H), 2.06-2.05 (m, 1H), 1.99-1.86 (m, 2H), 1.73-1.64 |
| | (m, 4H), 1.46 (s, 9H). |
| 256^{B} | tert-butyl 5-(6-chloro-1-(tetrahydro-2H-pyran-2-yl)-1H-pyrazolo[4,3- |
| | c]pyridin-3-yl)-2,5-diazabicyclo[2.2.1]heptane-2-carboxylate |
| | 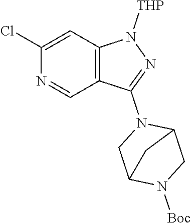 |
| | Amine: tert-butyl 2,5-diazabicyclo[2.2.1]heptane-2-carboxylate |
| | Yellow solid (402 mg, 67%); ¹H NMR (400 MHz, CDCl₃) δ: 8.65 (s, 1H), |
| | 7.30 (s, 1H), 5.43-5.41 (m, 1H), 4.68-4.67 (m, 2H), 4.01-4.00 (m, 1H), |
| | 3.87-3.80 (m, 1H), 3.70-3.51 (m, 3H), 3.41-3.40 (m, 1H), 2.41-2.40 (m, |
| | 1H), 2.10-2.09 (m, 1H), 1.98-1.93 (m, 3H), 1.71-1.64 (m, 3H), 1.46-1.41 |
| | (m, 9H). |
| 257^{B} | 6-(6-chloro-1-(tetrahydro-2H-pyran-2-yl)-1H-pyrazolo[4,3-c]pyridin-3-yl)- |
| | 3,3-difluoro-1-oxa-6-azaspiro[3.3]heptane |
| | 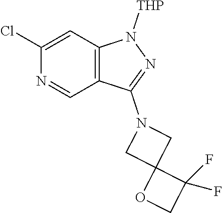 |
| | Amine: 3,3-difluoro-1-oxa-6-azaspiro[3.3]heptane |
| | Yellow solid (200 mg, 65%); LCMS m/z = 371.1 [M + H]⁺. |

A. 1:1 Xantphos/Pd₂(dba)₃ was used instead of Xantphos Pd G3
B. Ruphos Pd G3 was used instead of Xantphos Pd G3

### Preparation 258: 6-chloro-3-(3,4-dimethylpiperazin-1-yl)-1H-pyrazolo[4,3-c]pyridine hydrochloride

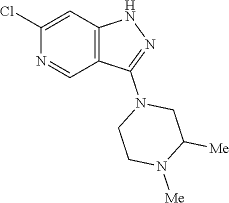 [SMILES: CC1CN(c2n[nH]c3cc(Cl)ncc23)CCN1C]

6-Chloro-3-(3,4-dimethylpiperazin-1-yl)-1-(tetrahydro-2H-pyran-2-yl)-1H-pyrazolo[4,3-c]pyridine (Preparation 229, 150 mg, 0.429 mmol) and HCl/EtOAc (4 M, 0.5 mL) in DCM (1 mL) was stirred at 20° C. for 4 h. The mixture evaporated to dryness in vacuo to give 6-chloro-3-(3,4-dimethylpiperazin-1-yl)-1H-pyrazolo[4,3-c]pyridine as a yellow solid (170 mg, crude) which was used without further purification. LCMS m/z=266.1 [M+H]⁺.

### Preparation 259: 6-chloro-3-(3-methoxypiperidin-1-yl)-1H-pyrazolo[4,3-c]pyridine trifluoroacetate

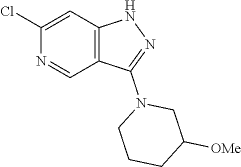 [SMILES: COC1CCCN(c2n[nH]c3cc(Cl)ncc23)C1]

To a solution of 6-chloro-3-(3-methoxypiperidin-1-yl)-1-(tetrahydro-2H-pyran-2-yl)-1H-pyrazolo[4,3-c]pyridine (Preparation 230, 259 mg, 0.738 mmol) in DCM (5 mL) was added TFA (84.2 mg, 0.738 mmol) and the resulting mixture was stirred at 25° C. for 16 h. The reaction mixture was evaporated to dryness and the residue diluted with H₂O (10 mL) and extracted with EtOAc (10 mL×3). The combined organics were washed with brine (10 mL×2), dried (Na₂SO₄) and evaporated to dryness. The residue was purified by chromatography on silica gel (PE/EtOAc=3/1) to give 6-chloro-3-(3-methoxypiperidin-1-yl)-1H-pyrazolo[4,3-c]pyridine as a yellow solid (136 mg, 69%). ¹H NMR (400 MHz, CDCl₃) δ: 8.93-8.90 (m, 1H), 7.34 (s, 1H), 3.84-3.81 (m, 1H), 3.65-3.59 (m, 1H), 3.52-3.50 (m, 1H), 3.42 (s, 3H) 3.37-3.32 (m, 2H), 2.01-1.98 (m, 2H), 1.72-1.67 (m, 2H).

### Preparation 260-276

The title compounds were prepared by acid mediated deprotection of the appropriate THP-protected compound using one of 2 methods previously described. Method 1; analogous method to that described for Preparation 258 or Method-2; analogous method to that described for Preparation 259.

| Preparation | |
|---|---|
| No | Name/Structure/R¹R²NH/Data |
| 260 | 6-chloro-3-(3-methoxypyrrolidin-1-yl)-1H-pyrazolo[4,3-c]pyridine |
| | hydrochloride |
| | 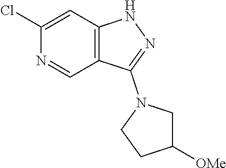 [SMILES: COC1CCN(c2n[nH]c3cc(Cl)ncc23)C1] |
| | SM: 6-chloro-3-(3-methoxypyrrolidin-1-yl)-1-(tetrahydro-2H-pyran-2-yl)- |
| | 1H-pyrazolo[4,3-c]pyridine (Preparation 231). |
| | Method 1. Yellow oil (200 mg); LCMS m/z = 253.3 [M + H]⁺. |
| 261 | 1-(6-chloro-1H-pyrazolo[4,3-c]pyridin-3-yl)pyrrolidin-3-ol hydrochloride |
| | 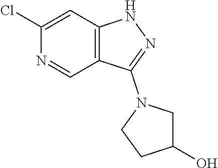 [SMILES: OC1CCN(c2n[nH]c3cc(Cl)ncc23)C1] |
| | SM: 1-(6-chloro-1-(tetrahydro-2H-pyran-2-yl)-1H-pyrazolo[4,3-c]pyridin- |
| | 3-yl)pyrrolidin-3-ol (Preparation 232) |
| | Method 1. Yellow solid (263 mg); ¹H NMR (400 MHz, MeOH-d₄) δ: 9.10 |
| | (s, 1H), 7.63 (s, 1H), 3.87-3.81 (m, 4H), 3.66-3.61 (m, 2H), 2.28-2.24 (m, |
| | 1H). |
| 262 | 1-(6-chloro-1H-pyrazolo[4,3-c]pyridin-3-yl)-3-methylpyrrolidin-3-ol |
| | hydrochloride |
| | 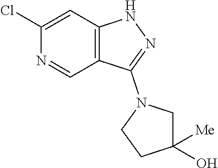 [SMILES: CC1(O)CCN(c2n[nH]c3cc(Cl)ncc23)C1] |
| | SM: 1-(6-chloro-1-(tetrahydro-2H-pyran-2-yl)-1H-pyrazolo[4,3-c]pyridin- |
| | 3-yl)-3-methylpyrrolidin-3-ol (Preparation 233) |
| | Method 1. White solid (110 mg); ¹H NMR (500 MHz, DMSO-d₆) δ: 8.85 |
| | (s, 1H), 7.36 (s, 1H), 3.71-3.65 (m, 2H), 3.49-3.45 (m, 2H), 1.99-1.93 (m, |
| | 2H), 1.37 (s, 3H) |
| 263 | 6-chloro-3-(3,3-difluoroazetidin-1-yl)-1H-pyrazolo[4,3-c]pyridine |
| | trifluoroacetate |
| | 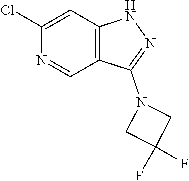 [SMILES: FC1(F)CN(c2n[nH]c3cc(Cl)ncc23)C1] |
| | SM: 6-chloro-3-(3,3-difluoroazetidin-1-yl)-1-(tetrahydro-2H-pyran-2-yl)- |
| | 1H-pyrazolo[4,3-c]pyridine (Preparation 250) |
| | Method 2. Brown oil (35 mg); LCMS m/z = 245.0 [M + H]⁺. |
| 264 | 6-chloro-3-(3-fluoroazetidin-1-yl)-1H-pyrazolo[4,3-c]pyridine |
| | trifluoroacetate |
| | 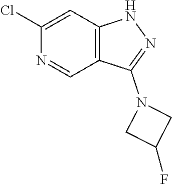 [SMILES: FC1CN(c2n[nH]c3cc(Cl)ncc23)C1] |
| | SM: 6-chloro-3-(3-fluoroazetidin-1-yl)-1-(tetrahydro-2H-pyran-2-yl)-1H- |
| | pyrazolo[4,3-c]pyridine (Preparation 234) |
| | Method 2. Yellow solid (98 mg, 75%); LCMS m/z = 227.1 [M + H]⁺. |
| 265 | 6-chloro-3-(2-methylazetidin-1-yl)-1H-pyrazolo[4,3-c]pyridine |
| | trifluoroacetate |
| | 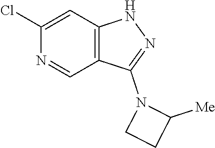 [SMILES: CC1CCN1c1n[nH]c2cc(Cl)ncc12] |
| | SM: 6-chloro-3-(2-methylazetidin-1-yl)-1-(tetrahydro-2H-pyran-2-yl)-1H- |
| | pyrazolo[4,3-c]pyridine (Preparation 235) |
| | Method 2. Yellow solid (40 mg); ¹H NMR (500 MHz, MeOH-d₄) δ: 8.62 (s, |
| | 1H), 7.31 (s, 1H), 4.51-4.48 (m, 1H), 4.25-4.22 (m, 1H), 4.00-3.98 (m, 1H), |
| | 2.56-2.52 (m, 1H), 2.21-2.17 (m, 1H), 1.52 (d, 3H). |
| 266 | 6-chloro-3-(3-methoxyazetidin-1-yl)-1H-pyrazolo[4,3-c]pyridine |
| | trifluoroacetate |
| | 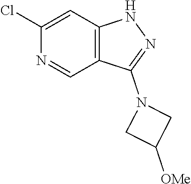 [SMILES: COC1CN(c2n[nH]c3cc(Cl)ncc23)C1] |
| | SM: 6-chloro-3-(3-methoxyazetidin-1-yl)-1-(tetrahydro-2H-pyran-2-yl)- |
| | 1H-pyrazolo[4,3-c]pyridine (Preparation 236) |
| | Method 2. Yellow oil (318 mg); LCMS m/z = 239.0 [M + H]⁺. |
| 267 | 1-(6-chloro-1H-pyrazolo[4,3-c]pyridin-3-yl)azetidin-3-ol trifluoroacetate |
| | 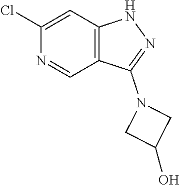 [SMILES: OC1CN(c2n[nH]c3cc(Cl)ncc23)C1] |
| | SM: 1-(6-chloro-1-(tetrahydro-2H-pyran-2-yl)-1H-pyrazolo[4,3-c]pyridin- |
| | 3-yl)azetidin-3-ol (Preparation 237) |
| | Method 2. Yellow solid (50 mg, 36%); LCMS m/z = 225.0 [M + H]⁺. |
| 268 | 1-(6-chloro-1H-pyrazolo[4,3-c]pyridin-3-yl)-3-methylazetidin-3-ol |
| | trifluoroacetate |
| | 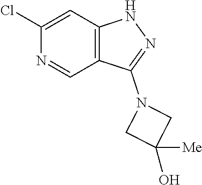 [SMILES: CC1(O)CN(c2n[nH]c3cc(Cl)ncc23)C1] |
| | SM: 1-(6-chloro-1-(tetrahydro-2H-pyran-2-yl)-1H-pyrazolo[4,3-c]pyridin- |
| | 3-yl)-3-methylazetidin-3-ol (Preparation 238) |
| | Method 2. HPLC-C (10-40% MeCN). |
| | White solid (60 mg, 27%); LCMS m/z = 239.1 [M + H]⁺. |
| 269 | 6-chloro-3-(3-methoxy-3-methylazetidin-1-yl)-1H-pyrazolo[4,3-c]pyridine |
| | trifluoroacetate |
| | 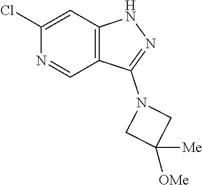 [SMILES: COC1(C)CN(c2n[nH]c3cc(Cl)ncc23)C1] |
| | SM: 6-chloro-3-(3-methoxy-3-methylazetidin-1-yl)-1-(tetrahydro-2H- |
| | pyran-2-yl)-1H-pyrazolo[4,3-c]pyridine (Preparation 239) |
| | Method 2. Yellow oil (100 mg, 71%); LCMS m/z = 253.3 [M + H]⁺. |
| 270 | 6-chloro-3-(3-(trifluoromethoxy)azetidin-1-yl)-1H-pyrazolo[4,3-c]pyridine |
| | trifluoroacetate |
| | 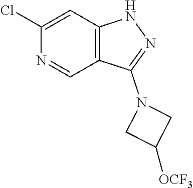 [SMILES: FC(F)(F)OC1CN(c2n[nH]c3cc(Cl)ncc23)C1] |
| | SM: 6-chloro-1-(tetrahydro-2H-pyran-2-yl)-3-(3- |
| | (trifluoromethoxy)azetidin-1-yl)-1H-pyrazolo[4,3-c]pyridine (Preparation |
| | 240) |
| | Method 2. Yellow oil (50 mg%); ¹H NMR (400 MHz, MeOH-d₄) δ: 8.65 (s, |
| | 1H), 7.34 (s, 1H), 4.60-4.55 (m, 3H), 4.28-4.24 (m, 2H) |
| 271 | 1-(6-chloro-1H-pyrazolo[4,3-c]pyridin-3-yl)-N,N-dimethylazetidin-3-amine |
| | trifluoroacetate |
| | 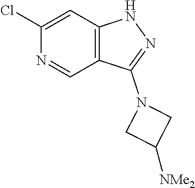 [SMILES: CN(C)C1CN(c2n[nH]c3cc(Cl)ncc23)C1] |
| | SM: 1-(6-chloro-1-(tetrahydro-2H-pyran-2-yl)-1H-pyrazolo[4,3-c]pyridin- |
| | 3-yl)-N,N-dimethylazetidin-3-amine (Preparation 241) |
| | Method 2. Brown solid (250 mg, 65%); LCMS m/z = 252.0 [M + H]⁺. |
| 272 | 6-(6-chloro-1H-pyrazolo[4,3-c]pyridin-3-yl)-2-oxa-6-azaspiro[3.3]heptane |
| | trifluoroacetate |
| | 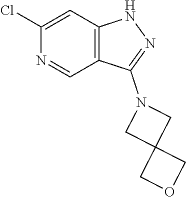 [SMILES: Clc1cc2[nH]nc(N3CC4(COC4)C3)c2cn1] |
| | SM: 6-(6-chloro-1-(tetrahydro-2H-pyran-2-yl)-1H-pyrazolo[4,3-c]pyridin- |
| | 3-yl)-2-oxa-6-azaspiro[3.3 ]heptane (Preparation 242) |
| | Method 2. Yellow solid (200 mg, 67%); ¹H NMR (400 MHz, CDCl₃) δ: |
| | 8.65 (s, 1H), 7.23 (d, 1H), 4.90-4.89 (m, 4H), 4.39 (s, 4H). |
| 273 | 1-(6-chloro-1H-pyrazolo[4,3-c]pyridin-3-yl)-3-fluoroazetidine-3- |
| | carbonitrile trifluoroacetate |
| | 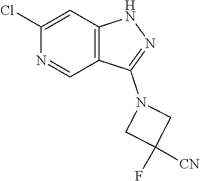 [SMILES: N#CC1(F)CN(c2n[nH]c3cc(Cl)ncc23)C1] |
| | SM: 1-(6-chloro-1-(tetrahydro-2H-pyran-2-yl)-1H-pyrazolo[4,3-c]pyridin- |
| | 3-yl)-3-fluoroazetidine-3-carbonitrile (Preparation 251) |
| | Method 2. White solid (73 mg, 82%); LCMS m/z = 252.0 [M + H]⁺. |
| 274 | 1-(6-chloro-1H-pyrazolo[4,3-c]pyridin-3-yl)-6-oxa-1-azaspiro[3.3]heptane |
| | trifluoroacetate |
| | 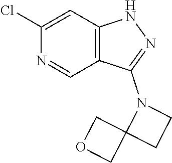 [SMILES: Clc1cc2[nH]nc(N3CCC34COC4)c2cn1] |
| | SM: 1-(6-chloro-1-(tetrahydro-2H-pyran-2-yl)-1H-pyrazolo[4,3-c]pyridin- |
| | 3-yl)-6-oxa-1-azaspiro[3.3 ]heptane (Preparation 244) |
| | Method 2. White solid (35 mg, 47%); ¹H NMR (500 MHz, CDCl₃) δ: 10.13 |
| | (br s, 1H), 8.78 (s, 1H), 5.45 (d, 2H), 4.86 (d, 2H), 4.15 (t, 2H), 2.73 (t, 2H). |
| 275 | 1-(6-chloro-1H-pyrazolo[4,3-c]pyridin-3-yl)-N,N,3-trimethylpyrrolidin-3- |
| | amine hydrochloride |
| | 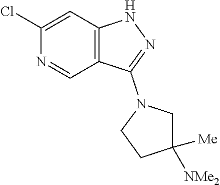 [SMILES: CN(C)C1(C)CCN(c2n[nH]c3cc(Cl)ncc23)C1] |
| | SM: 1-(6-chloro-1-(tetrahydro-2H-pyran-2-yl)-1H-pyrazolo[4,3-c]pyridin- |
| | 3-yl)-N,N,3-trimethylpyrrolidin-3-amine (Preparation 245) |
| | Method 1. Yellow solid (135 mg, crude); ¹H NMR (500 MHz, MeOH-d₄) δ: |
| | 9.24 (s, 1H), 7.75 (s, 1H), 2.56-2.42 (m, 4H), 2.04-2.01 (m, 6H), 1.68-1.66 |
| | (m, 1H) 1.60-1.59 (m, 4H). |
| 276 | 6-(6-chloro-1H-pyrazolo[4,3-c]pyridin-3-yl)-3,3-difluoro-1-oxa-6- |
| | azaspiro[3.3]heptane trifluoroacetate |
| | 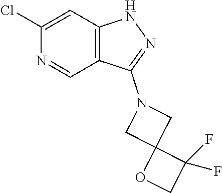 [SMILES: FC1(F)COC12CN(c1n[nH]c3cc(Cl)ncc13)C2] |
| | SM: 6-(6-chloro-1-(tetrahydro-2H-pyran-2-yl)-1H-pyrazolo[4,3-c]pyridin- |
| | 3-yl)-3,3-difluoro-1-oxa-6-azaspiro[3.3]heptane (Preparation 257) |
| | Method 2. White solid (40 mg, 21%); ¹H NMR (500 MHz, CDCl₃) δ: 9.44 |
| | (br s, 1H), 8.63 (s, 1H), 7.23 (s, 1H), 4.80 (t, 2H), 4.69 (d, 2H), 4.30 (d, |
| | 2H). |

Preparation 277: 2-(6-chloro-1H-pyrazolo[4,3-c]pyridin-3-yl)-2,5-diazabicyclo [2.2.2]octane hydrochloride

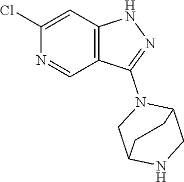 [SMILES: Clc1cc2[nH]nc(N3CC4CCC3CN4)c2cn1]

A solution of tert-butyl 5-(6-chloro-1-(tetrahydro-2H-pyran-2-yl)-1H-pyrazolo[4,3-c]pyridin-3-yl)-2,5-diazabicyclo[2.2.2]octane-2-carboxyl)ate (Preparation 252, 171.2 mg, 0.382 mmol) in HCl/dioxane (4 M, 3.0 mL) was stirred at 25° C. for 1 h. The mixture was concentrated to give 2-(6-chloro-H-pyrazolo[4,3-c]pyridin-3-yl)-2,5-diazabicyclo[2.2.2]octane hydrochloride (150.3 mg, crude) as a yellow solid which was used without further purification. LCMS m/z=264.1 [M+H]⁺.

### Preparations 278-279

The title compounds were prepared from the appropriate protected pyrazolo[4,3-c]pyri dine, using an analogous method to that described for Preparation 277.

| Preparation | |
|---|---|
| No | Name/Structure/SM/Data |
| 278 | 1-(6-chloro-1H-pyrazolo[4,3-c]pyridin-3-yl)pyrrolidin-3-amine |
| | hydrochloride |
| | 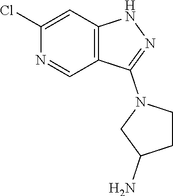 [SMILES: NC1CCN(c2n[nH]c3cc(Cl)ncc23)C1] |
| | tert-butyl (1-(6-chloro-1-(tetrahydro-2H-pyran-2-yl)-1H-pyrazolo[4,3- |
| | c]pyridin-3-yl)pyrrolidin-3-yl)carbamate (Preparation 243) |
| | Yellow solid (1.4 g, crude); LCMS m/z = 238.1 [M + H]⁺. |
| 279 | 6-chloro-3-(hexahydropyrrolo[3,4-b]pyrrol-5(1H)-yl)-1H-pyrazolo[4,3- |
| | c]pyridine hydrochloride |
| | 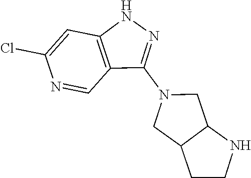 [SMILES: Clc1cc2[nH]nc(N3CC4CCNC4C3)c2cn1] |
| | SM: tert-butyl 5-(6-chloro-1-(tetrahydro-2H-pyran-2-yl)-1H-indazol-3- |
| | yl)hexahydropyrrolo[3,4-b]pyrrole-1(2H)-carboxylate (Preparation 247) |
| | Yellow solid (62 mg, crude); ¹H NMR (500 MHz, MeOH-d₄) δ: 9.07 (s, |
| | 1H), 7.58 (s, 1H), 4.47-4.45 (m, 1H), 4.13 (d, 1H), 3.82-3.80 (m, 1H), 3.70- |
| | 3.66 (m, 2H), 3.40-3.36 (m, 3H), 2.43-2.38 (m, 1H), 2.16-2.11 (m, 1H). |

### Preparation 280: 1-(6-chloro-1H-pyrazolo[4,3-c]pyridin-3-yl)-3-fluoropyrrolidine-3-carbonitrile trifluoroacetate

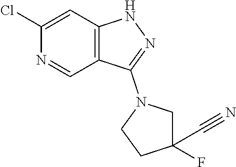 [SMILES: N#CC1(F)CCN(c2n[nH]c3cc(Cl)ncc23)C1]

Part A: 1-(6-Chloro-1-(tetrahydro-2H-pyran-2-yl)-1H-pyrazolo[4,3-c]pyridin-3-yl)-3-fluoropyrrolidine-3-carbonitrile was obtained as a white solid, 60.5 mg, 41.9% yield, from 6-chloro-3-iodo-1-(tetrahydro-2H-pyran-2-yl)-1H-pyrazolo[4,3-c]pyridine and 3-fluoropyrrolidine-3-carbonitrile, following a similar procedure to that described in Preparation 249. LCMS m/z=250.1 [M+H]⁺ Part B: 1-(6-Chloro-1H-pyrazolo[4,3-c]pyridin-3-yl)-3-fluoropyrrolidine-3-carbonitrile was obtained as a white solid, 35.2 mg, 83.5% yield, from 1-(6-chloro-1-(tetrahydro-2H-pyran-2-yl)-1H-pyrazolo[4,3-c]pyridin-3-yl)-3-fluoropyrrolidine-3-carbonitrile, following a similar procedure to that described in Preparation 259. LCMS m/z=266.0 [M+H]⁺

### Preparation 281: 1-(6-chloro-1H-pyrazolo[4,3-c]pyridin-3-yl)-3-methylpyrrolidine-3-carbonitrile trifluoroacetate

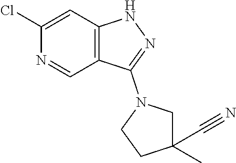 [SMILES: CC1(C#N)CCN(c2n[nH]c3cc(Cl)ncc23)C1]

1-(6-Chloro-1H-pyrazolo[4,3-c]pyridin-3-yl)-3-methylpyrrolidine-3-carbonitrile trifluoroacetate was obtained as a white solid, 160 mg, 70.5% yield, from 1-(6-chloro-1-(tetrahydro-2H-pyran-2-yl)-1H-pyrazolo[4,3-c]pyridin-3-yl)-3-methylpyrrolidine-3-carbonitrile, following a similar procedure to that described in Preparation 259. LCMS/z=262.1 [M+H]⁺

### Preparation 282: 6-chloro-3-(4-methylpiperazin-1-yl)-1H-pyrazolo[4,3-c]pyridine

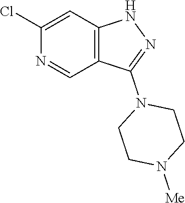 [SMILES: CN1CCN(c2n[nH]c3cc(Cl)ncc23)CC1]

6-Chloro-3-(4-methylpiperazin-1-yl)-1H-pyrazolo[4,3-c]pyridine was obtained from 6-chloro-3-iodo-1-(tetrahydro-2H-pyran-2-yl)-1H-pyrazolo[4,3-c]pyridine and 4-methylpiperazine, following a similar procedure to that described in Preparation 280.

### Preparation 283: 3-(3,6-diazabicyclo[3.2.0]heptan-6-yl)-6-chloro-1H-pyrazolo[4,3-c]pyridine

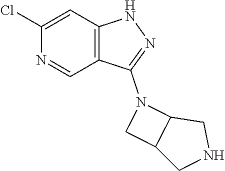 [SMILES: Clc1cc2[nH]nc(N3CC4CNCC43)c2cn1]

To a solution of tert-butyl 6-(6-chloro-1-(tetrahydro-2H-pyran-2-yl)-1H-pyrazolo[4,3-c]pyridin-3-yl)-3,6-diazabicyclo[3.2.0]heptane-3-carboxylate (Preparation 246, 197 mg, 0.454 mmol) in DCM (5 mL) was added TFA (1.3 mL) and the mixture stirred at 25° C. for 16 h. Saturated NaHCO₃ was added to adjust the pH to ˜7 and the mixture diluted with water (5 mL) and extracted with EtOAc (10 mL×3). The combined extracts were evaporated to dryness in vacuo and the residue purified by prep-HPLC-F (10-40% MeCN) to give 3-(3,6-diazabicyclo[3.2.0]heptan-6-yl)-6-chloro-1H-pyrazolo[4,3-c]pyridine as a white solid (85 mg, 75%). LCMS m/z=250.0 [M+H]⁺.

### Preparation 284: 2-(6-chloro-1H-pyrazolo[4,3-c]pyridin-3-yl)-5-methyl-2,5-diazabicyclo[2.2.2]octane

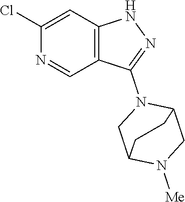 [SMILES: CN1CC2CCC1CN2c1n[nH]c2cc(Cl)ncc12]

To a solution of 2-(6-chloro-1H-pyrazolo[4,3-c]pyridin-3-yl)-2,5-diazabicyclo[2.2.2]octane hydrochloride (Preparation 277, 140.3 mg, 0.467 mmol) in MeOH (5.0 mL) was added NaBH₃CN (146.9 mg, 2.34 mmol) and (CHO)ₙ (1.7 g, 1.40 mmol) and the mixture stirred at 25° C. for 1 h. The reaction mixture was purified by prep-HPLC-C (17-47% MeCN) to give 2-(6-chloro-1H-pyrazolo[4,3-c]pyridin-3-yl)-5-methyl-2,5-diazabicyclo[2.2.2]octane as a pale yellow solid (60.1 mg, 46%). LCMS m/z=278.1 [M+H]⁺.

### Preparation 285: 1-(6-chloro-1H-pyrazolo[4,3-c]pyridin-3-yl)-N,N-dimethylpyrrolidin-3-amine

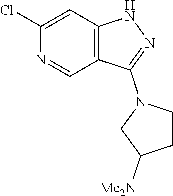 [SMILES: CN(C)C1CCN(c2n[nH]c3cc(Cl)ncc23)C1]

1-(6-Chloro-1H-pyrazolo[4,3-c]pyridin-3-yl)-N,N-dimethylpyrrolidin-3-amine was prepared as a yellow solid (250 mg, 22%) from 1-(6-chloro-1H-pyrazolo[4,3-c]pyridin-3-yl)pyrrolidin-3-amine hydrochloride (Preparation 278) using an analogous method to that described for Preparation 284. LCMS m/z=266.1 [M+H]⁺.

### Preparation 286: 6-chloro-3-(3-methyl-3,6-diazabicyclo[3.2.0]heptan-6-yl)-1H-pyrazolo[4,3-c]pyridine

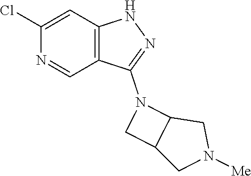 [SMILES: CN1CC2CN(c3n[nH]c4cc(Cl)ncc34)C2C1]

6-Chloro-3-(3-methyl-3,6-diazabicyclo[3.2.0]heptan-6-yl)-1H-pyrazolo[4,3-c]pyridine was prepared as a yellow solid (55 mg, 42%) from 3-(3,6-diazabicyclo[3.2.0]heptan-6-yl)-6-chloro-1H-pyrazolo[4,3-c]pyridine (Preparation 280) using an analogous method to that described for Preparation 284. LCMS m/z=264.0 [M+H]⁺.

### Preparation 287: 6-chloro-3-(1-methylhexahydropyrrolo[3,4-b]pyrrol-5(1H)-yl)-1H-pyrazolo[4,3-c]pyridine

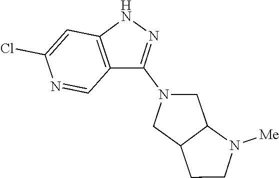 [SMILES: CN1CCC2CN(c3n[nH]c4cc(Cl)ncc34)CC21]

6-Chloro-3-(1-methylhexahydropyrrolo[3,4-b]pyrrol-5(1H)-yl)-1H-pyrazolo[4,3-c]pyridine was prepared as a yellow solid (35 mg, 55%) from 6-chloro-3-(hexahydropyrrolo[3,4-b]pyrrol-5(1H)-yl)-1H-pyrazolo[4,3-c]pyridine hydrochloride (Preparation 279) using an analogous method to that described for Preparation 284. ¹H NMR (400 MHz, MeOH-d₄) δ: 8.93 (s, 1H), 7.39 (s, 1H), 4.22-4.17 (m, 2H), 3.83 (d, 1H), 3.51-3.46 (m, 1H), 3.43-3.39 (m, 3H), 3.28-3.25 (m, 1H), 3.25 (s, 3H), 2.67-2.63 (m, 1H), 2.02-1.96 (m, 1H).

### Preparation 288: 3-(6-chloro-1H-pyrazolo[4,3-c]pyridin-3-yl)-6-methyl-3,6-diazabicyclo[3.1.1]heptane

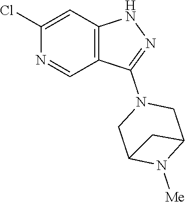 [SMILES: CN1C2CC1CN(c1n[nH]c3cc(Cl)ncc13)C2]

Part 1: 3-(6-Chloro-1H-pyrazolo[4,3-c]pyridin-3-yl)-3,6-diazabicyclo[3.1.1]heptane hydrochloride was prepared as a yellow solid (80 mg, crude) from tert-butyl 3-(6-chloro-1-(tetrahydro-2H-pyran-2-yl)-1H-pyrazolo[4,3-c]pyridin-3-yl)-3,6-diazabicyclo[3.1.1]heptane-6-carboxylate (Preparation 253) using an analogous method to that described for Preparation 277

Part 2: 3-(6-Chloro-1H-pyrazolo[4,3-c]pyridin-3-yl)-6-methyl-3,6-diazabicyclo[3.1.1]heptane was prepared from 3-(6-chloro-1H-pyrazolo[4,3-c]pyridin-3-yl)-3,6-diazabicyclo[3.1.1]heptane hydrochloride (Part 1, 80 mg) using an analogous method to that described for Preparation 284. Yellow solid (50 mg, 59%); ¹H NMR (500 MHz, CDCl₃) δ: 9.10 (s, 1H), 8.92 (s, 1H), 7.20 (s, 1H), 3.96 (d, 2H), 3.75 (d, 2H), 3.70 (d, 2H), 2.76-2.71 (m, 1H), 2.26 (s, 3H), 1.75 (d, 1H).

### Preparation 289: 6-chloro-3-(5-methylhexahydropyrrolo[3,4-c]pyrrol-2(1H)-yl)-1H-pyrazolo[4,3-c]pyridine

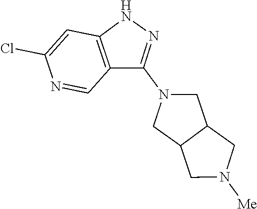 [SMILES: CN1CC2CN(c3n[nH]c4cc(Cl)ncc34)CC2C1]

6-Chloro-3-(5-methylhexahydropyrrolo[3,4-c]pyrrol-2(1H)-yl)-1H-pyrazolo[4,3-c]pyridine was prepared as a white solid (47.7 mg, 52%) from tert-butyl 5-(6-chloro-1-(tetrahydro-2H-pyran-2-yl)-1H-pyrazolo[4,3-c]pyridin-3-yl)hexahydropyrrolo[3,4-c]pyrrole-2(1H)-carboxylate (Preparation 248) using an analogous 2-part method as described for Preparation 288. LCMS m/z=278.1 [M+H]⁺.

### Preparation 290: 6-chloro-3-(8-methyl-3,8-diazabicyclo[3.2.1]octan-3-yl)-1H-pyrazolo[4,3-c]pyridine

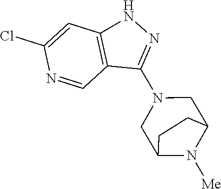 [SMILES: CN1C2CCC1CN(c1n[nH]c3cc(Cl)ncc13)C2]

6-Chloro-3-(8-methyl-3,8-diazabicyclo[3.2.1]octan-3-yl)-1H-pyrazolo[4,3-c]pyridine was prepared as a yellow solid (183 mg, 58%) from tert-butyl 3-(6-chloro-1-(tetrahydro-2H-pyran-2-yl)-1H-pyrazolo[4,3-c]pyridin-3-yl)-3, 8-diazabicyclo[3.2.1]octane-8-carboxylate (Preparation 254) using an analogous 2-part method as described for Preparation 289. ¹H NMR (500 MHz, DMSO-d₆) δ: 12.70 (s, 1H), 10.87 (s, 1H), 6.70 (s, 1H), 7.42 (s, 1H), 3.91-3.88 (m, 2H), 3.56-3.54 (m, 2H), 2.75-2.73 (m, 4H), 2.23-2.21 (m, 2H), 2.30-2.07 (m, 2H).

### Preparation 291: 6-chloro-3-(3-methyl-3,8-diazabicyclo[3.2.1]octan-8-yl)-1H-pyrazolo[4,3-c]pyridine

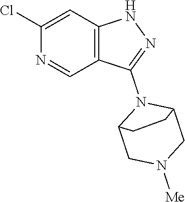 [SMILES: CN1CC2CCC(C1)N2c1n[nH]c2cc(Cl)ncc12]

6-Chloro-3-(3-methyl-3,8-diazabicyclo[3.2.1]octan-8-yl)-1H-pyrazolo[4,3-c]pyridine was prepared as a white solid (65 mg, 70%) from tert-butyl 8-(6-chloro-1-(tetrahydro-2H-pyran-2-yl)-1H-pyrazolo[4,3-c]pyridin-3-yl)-3, 8-diazabicyclo[3.2.1]octane-3-carboxylate (Preparation 255) using an analogous 2-part method as described for Preparation 288. LCMS m/z=278.1 [M+H]⁺.

### Preparation 292: 6-chloro-3-(5-methyl-2,5-diazabicyclo[2.2.1]heptan-2-yl)-1H-pyrazolo[4,3-c]pyridine

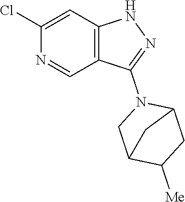 [SMILES: CC1CC2CC1CN2c1n[nH]c2cc(Cl)ncc12]

6-Chloro-3-(5-methyl-2,5-diazabicyclo[2.2.1]heptan-2-yl)-1H-pyrazolo[4,3-c]pyridine was prepared as a yellow solid (123 mg, 47%) from tert-butyl 5-(6-chloro-1-(tetrahydro-2H-pyran-2-yl)-1H-pyrazolo[4,3-c]pyridin-3-yl)-2,5-diazabicyclo[2.2.1]heptane-2-carboxylate (Preparation 256) using an analogous 2-part method as described for Preparation 288. LCMS m/z=264.0 [M+H]⁺.

### Preparation 293: 6-chloro-1-(4-(1,1-difluoroethyl)pyrimidin-2-yl)-3-(3,4-dimethylpiperazin-1-yl)-1H-pyrazolo[4,3-c]pyridine

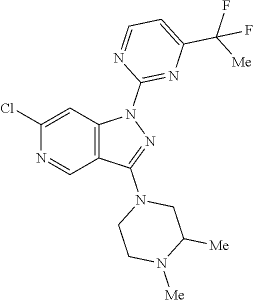 [SMILES: CC1CN(c2nn(-c3nccc(C(C)(F)F)n3)c3cc(Cl)ncc23)CCN1C]

A mixture of 6-chloro-3-(3,4-dimethylpiperazin-1-yl)-1H-pyrazolo[4,3-c]pyridine (Preparation 258, 170 mg, 0.640 mmol), 2-chloro-4-(1,1-difluoroethyl)pyrimidine (Preparation 178, 137 mg, 0.768 mmol) and Cs₂CO₃ (417 mg, 1.28 mmol) in DMSO (2 mL) was stirred at 70° C. for 2 h. The mixture was poured into water (10 mL) and the solids collected by filtration to afford 6-chloro-1-(4-(1,1-difluoroethyl)pyrimidin-2-yl)-3-(3,4-dimethylpiperazin-1-yl)-1H-pyrazolo[4,3-c]pyridine as a yellow solid (130 mg, 50%). ¹H NMR (400 MHz, CDCl₃) δ: 8.97 (d, 1H), 8.90 (s, 1H), 8.57 (s, 1H), 7.41 (d, 1H), 4.09-3.94 (m, 2H), 3.43-3.40 (m, 1H), 3.09-2.96 (m, 2H), 2.64-2.49 (m, 2H), 2.39 (s, 3H), 2.17-2.05 (m, 3H), 1.21 (d, 3H).

### Preparations 294 to 349

The title compounds were prepared from the appropriate pyrimidine (RCl) and the appropriate pyrazolo[4,3-c]pyridine (SM) in DMF or DMSO using an analogous method to that described for Preparation 293 using purification outlined in the table. RCl-1, 2-chloro-4-(1,1-difluoroethyl)pyrimidine (Preparation 178); RCl-2, 4-chloro-2-(1,1-difluoroethyl)pyrimidine (Preparation 184); RCl-3, 2-chloro-4-(1,1-difluoroethyl)-6-methylpyrimidine (Preparation 180); RCl-4: 4-chloro-6-(1,1-difluoroethyl)-2-methylpyrimidine (Preparation 213); RCl-5: 4,6-dichloro-2-(1,1-difluoroethyl)pyrimidine (Preparation 189); RCl-6: 4-chloro-6-(1,1-difluoroethyl)pyrimidine; RCl-7: 4-chloro-2-(1,1-difluoroethyl)-6-methylpyrimidine (Preparation 218).

| Preparation No | Name/Structure/R¹R²NH/Data |
|---|---|
| 294 | 6-chloro-1-(2-(1,1-difluoroethyl)pyrimidin-4-yl)-3-(3,4- |
| | dimethylpiperazin-1-yl)-1H-pyrazolo[4,3-c]pyridine |
| | 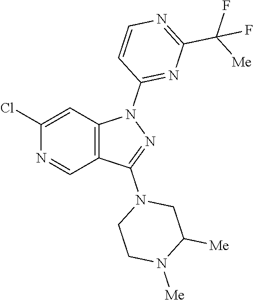 [SMILES: CC1CN(c2nn(-c3ccnc(C(C)(F)F)n3)c3cc(Cl)ncc23)CCN1C] |
| | SM: 6-chloro-3-(3,4-dimethylpiperazin-1-yl)-1H-pyrazolo[4,3- |
| | c]pyridine (Preparation 258); RCl-2 |
| | Yellow solid (100 mg, 54%). ¹H NMR (500 MHz, DMSO-d₆) δ: |
| | 9.23 (s, 1H), 8.92 (d, 1H), 8.58 (s, 1H), 7.84 (d, 1H), 4.06-3.95 (m, |
| | 2H), 3.39-3.37 (m, 1H), 3.23-3.22 (m, 1H), 2.89-2.82 (m, 2H), 2.36- |
| | 2.35 (m, 1H), 2.26 (s, 3H), 2.12 (t, 3H), 1.11 (d, 3H). |
| 295 | 4-(6-chloro-1-(4-(1,1-difluoroethyl)pyrimidin-2-yl)-1H- |
| | pyrazolo[4,3-c]pyridin-3-yl)-1-methylpiperazin-2-one |
| | 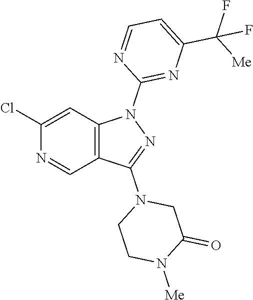 [SMILES: CN1CCN(c2nn(-c3nccc(C(C)(F)F)n3)c3cc(Cl)ncc23)CC1=O] |
| | SM: 4-(6-chloro-1H-pyrazolo[4,3-c]pyridin-3-yl)-1- |
| | methylpiperazin-2-one (Preparation 227), RCl-1 |
| | SiO₂ (100% EtOAc); Yellow solid (82.6 mg, 54%). ¹H NMR (500 MHz, |
| | CDCl₃) δ: 8.97 (d, 1H), 8.93 (s, 1H), 8.59 (s, 1H), 7.44 (d, |
| | 1H), 4.39 (s, 2H), 3.99-3.97 (m, 2H), 3.63-3.61 (m, 2H), 3.09 (s, |
| | 3H), 2.11 (t, 3H). |
| 296 | 4-(6-chloro-1-(2-(1,1-difluoroethyl)pyrimidin-4-yl)-1H- |
| | pyrazolo[4,3-c]pyridin-3-yl)-1-methylpiperazin-2-one |
| | 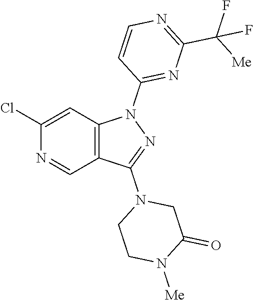 [SMILES: CN1CCN(c2nn(-c3ccnc(C(C)(F)F)n3)c3cc(Cl)ncc23)CC1=O] |
| | SM: 4-(6-chloro-1H-pyrazolo[4,3-c]pyridin-3-yl)-1- |
| | methylpiperazin-2-one (Preparation 227); RCl-2 |
| | SiO₂ (100% EtOAc); Yellow solid (202.8 mg, 63%). ¹H NMR (400 MHz, |
| | CDCl₃) δ: 8.89 (s, 1H), 8.79 (d, 1H), 8.76 (s, 1H), 7.79 (d, |
| | 1H), 4.34 (s, 2H), 3.96-3.93 (m, 2H), 3.64-3.61 (m, 2H), 3.09 (s, |
| | 3H), 2.13 (t, 3H). |
| 297 | 6-chloro-1-(4-(1,1-difluoroethyl)pyrimidin-2-yl)-3-(3- |
| | methoxypiperidin-1-yl)-1H-pyrazolo[4,3-c]pyridine |
| | 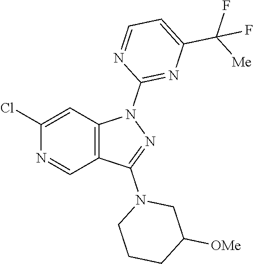 [SMILES: COC1CCCN(c2nn(-c3nccc(C(C)(F)F)n3)c3cc(Cl)ncc23)C1] |
| | SM: 6-chloro-3-(3-methoxypiperidin-1-yl)-1H-pyrazolo[4,3- |
| | c]pyridine (Preparation 259); RCl-1 |
| | SiO₂ (50% EtOAc/PE), Yellow solid (73 mg, 79%); ¹H NMR (500 MHz, |
| | CDCl₃) δ: 8.97 (d, 1H), 8.93 (s, 1H), 8.57 (s, 1H), 7.40 (d, |
| | 1H), 3.99-3.96 (m, 1H), 3.65-3.59 (m, 1H), 3.54-3.53 (m, 2H), 3.45 |
| | (s, 3H), 3.44-3.40 (m, 1H), 2.14-2.07 (m, 3H), 2.03-2.00 (m, 2H), |
| | 1.72-1.70 (m, 2H). |
| 298 | 6-chloro-1-(2-(1,1-difluoroethyl)pyrimidin-4-yl)-3-(3- |
| | methoxypiperidin-1-yl)-1H-pyrazolo[4,3-c]pyridine |
| | 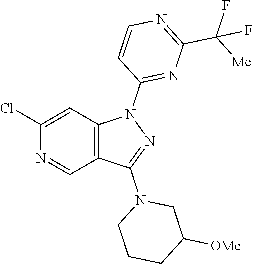 [SMILES: COC1CCCN(c2nn(-c3ccnc(C(C)(F)F)n3)c3cc(Cl)ncc23)C1] |
| | SM: 6-chloro-3-(3-methoxypiperidin-1-yl)-1H-pyrazolo[4,3- |
| | c]pyridine (Preparation 259); RCl-2 |
| | SiO₂ (50% EtOAc/PE), Yellow solid (80 mg, 74%); ¹H NMR (400 MHz, |
| | CDCl₃) δ: 8.91 (s, 1H), 8.77-8.74 (m, 2H), 7.80 (d, 1H), 3.96- |
| | 3.93 (m, 1H), 3.77-3.69 (m, 1H), 3.51-3.50 (m, 2H), 3.44-3.39 (m, |
| | 4H) 2.18-2.09 (m, 3H), 2.06-2.02 (t, 2H), 1.72-1.70 (m, 2H). |
| 299 | 6-chloro-1-(4-(1,1-difluoroethyl)pyrimidin-2-yl)-3-(3- |
| | methoxypyrrolidin-1-yl)-1H-pyrazolo[4,3-c]pyridine |
| | 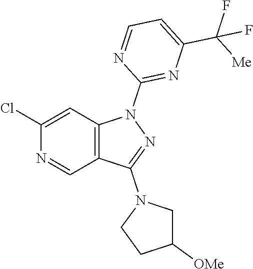 [SMILES: COC1CCN(c2nn(-c3nccc(C(C)(F)F)n3)c3cc(Cl)ncc23)C1] |
| | SM: 6-chloro-3-(3-methoxypyrrolidin-1-yl)-1H-pyrazolo[4,3- |
| | c]pyridine (Preparation 260); RCl-1 |
| | SiO₂ (50% EtOAc/PE), Yellow oil (70 mg, 45%); ¹H NMR (500 MHz, |
| | CDCl₃) δ: 8.97 (d, 1H), 8.91 (s, 1H), 8.56 (s, 1H), 7.38 (d, |
| | 1H), 4.18-4.17 (m, 1H), 3.96-3.90 (m, 4H), 3.40 (s, 3H), 2.29-2.27 |
| | (m, 1H), 2.19-2.15 (m, 1H), 2.11 (t, 3H). |
| 300 | 6-chloro-1-(2-(1,1-difluoroethyl)pyrimidin-4-yl)-3-(3- |
| | methoxypyrrolidin-1-yl)-1H-pyrazolo[4,3-c]pyridine |
| | 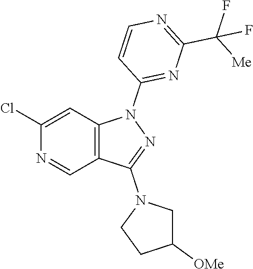 [SMILES: COC1CCN(c2nn(-c3ccnc(C(C)(F)F)n3)c3cc(Cl)ncc23)C1] |
| | SM: 6-chloro-3-(3-methoxypyrrolidin-1-yl)-1H-pyrazolo[4,3- |
| | c]pyridine (Preparation 260); RCl-2 |
| | SiO₂ (66% EtOAc/PE), Yellow oil (100 mg, 64%); ¹H NMR (500 MHz, |
| | CDCl₃) δ: 8.86 (s, 1H), 8.74 (d, 1H), 8.69 (s, 1H), 7.79 (d, |
| | 1H), 4.19-4.18 (m, 1H), 3.88-3.82 (m, 4H), 3.41 (s, 3H), 2.30-2.29 |
| | (m, 1H), 2.17-2.10 (m, 4H). |
| 301 | 1-(6-chloro-1-(4-(1,1-difluoroethyl)pyrimidin-2-yl)-1H- |
| | pyrazolo[4,3-c]pyridin-3-yl)pyrrolidin-3-ol |
| | 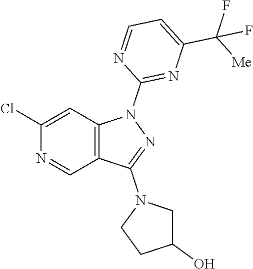 [SMILES: CC(F)(F)c1ccnc(-n2nc(N3CCC(O)C3)c3cnc(Cl)cc32)n1] |
| | SM: 1-(6-chloro-1H-pyrazolo[4,3-c]pyridin-3-yl)pyrrolidin-3-ol |
| | (Preparation 261); RCl-1 |
| | SiO₂ (100% EtOAc), white solid (75.5 mg, 39%); ¹H NMR (500 MHz, |
| | CDCl₃) δ: 8.96 (d, 1H), 8.91 (s, 1H), 8.56 (s, 1H), 7.38 (d, |
| | 1H), 4.74-4.73 (m, 1H), 4.04-3.96 (m, 3H), 3.95-3.84 (m, 1H), 2.67- |
| | 2.20 (m, 2H), 2.11 (t, 3H). |
| 302 | 1-(6-chloro-1-(2-(1,1-difluoroethyl)pyrimidin-4-yl)-1H- |
| | pyrazolo[4,3-c]pyridin-3-yl)pyrrolidin-3-ol |
| | 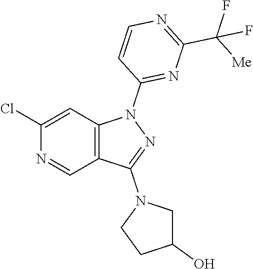 [SMILES: CC(F)(F)c1nccc(-n2nc(N3CCC(O)C3)c3cnc(Cl)cc32)n1] |
| | SM: 1-(6-chloro-1H-pyrazolo[4,3-c]pyridin-3-yl)pyrrolidin-3-ol |
| | (Preparation 261); RCl-2 |
| | SiO₂ (100% EtOAc), yellow solid (78 mg, 47%); ¹H NMR (400 MHz, |
| | CDCl₃) δ: 8.86 (s, 1H), 8.74 (d, 1H), 8.69 (s, 1H), 7.78 (d, |
| | 1H), 4.75-4.74 (m, 1H), 3.97-3.65 (m, 5H), 2.28-2.21 (m, 2H), 2.14 |
| | (t, 3H). |
| 303 | 1-(6-chloro-1-(4-(1,1-difluoroethyl)pyrimidin-2-yl)-1H- |
| | pyrazolo[4,3-c]pyridin-3-yl)-3-methylpyrrolidin-3-ol |
| | 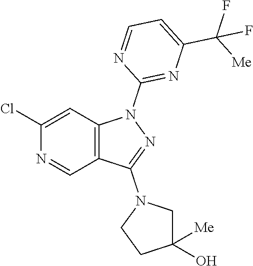 [SMILES: CC1(O)CCN(c2nn(-c3nccc(C(C)(F)F)n3)c3cc(Cl)ncc23)C1] |
| | SM: 1-(6-chloro-1H-pyrazolo[4,3-c]pyridin-3-yl)-3- |
| | methylpyrrolidin-3-ol (Preparation 262); RCl-1 |
| | SiO₂ (0-90% EtOAc/PE), white solid (90.6 mg, 58%); ¹H NMR (400 MHz, |
| | MeOH-d₄) δ: 8.98 (d, 1H), 8.94 (s, 1H), 8.57 (s, 1H) 7.51 (d, |
| | 1H), 4.60 (s, 1H), 4.02-3.93 (m, 2H), 3.81-3.69 (m, 2H), 2.15-2.10 |
| | (m, 1H), 2.14-2.08 (m, 3H), 2.05-2.01 (m, 1H), 1.53 (s, 3H). |
| 304 | 1-(6-chloro-1-(2-(1,1-difluoroethyl)pyrimidin-4-yl)-1H- |
| | pyrazolo[4,3-c]pyridin-3-yl)-3-methylpyrrolidin-3-ol |
| | 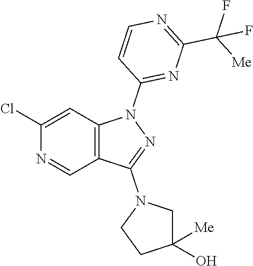 [SMILES: CC1(O)CCN(c2nn(-c3ccnc(C(C)(F)F)n3)c3cc(Cl)ncc23)C1] |
| | SM: 1-(6-chloro-1H-pyrazolo[4,3-c]pyridin-3-yl)-3- |
| | methylpyrrolidin-3-ol (Preparation 262): RCl-2 |
| | SiO₂ (0-90% EtOAc/PE), white solid (102.2 mg, 72%); ¹H NMR |
| | (400 MHz, MeOH-d₄) δ: 8.94 (s, 1H), 8.78 (d, 1H), 8.68 (s, 1H), |
| | 7.85 (d, 1H), 3.99-3.90 (m, 2H), 3.76-3.66 (m, 2H), 2.16-2.10 (m, |
| | 3H), 2.05-2.01 (m, 2H), 1.53 (s, 3H). |
| 305 | 1-(6-chloro-1-(2-(1,1-difluoroethyl)pyrimidin-4-yl)-1H- |
| | pyrazolo[4,3-c]pyridin-3-yl)-3-fluoropyrrolidine-3-carbonitrile |
| | 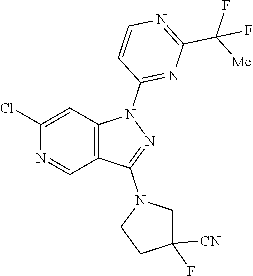 [SMILES: CC(F)(F)c1nccc(-n2nc(N3CCC(F)(C#N)C3)c3cnc(Cl)cc32)n1] |
| | SM: 1-(6-chloro-1H-pyrazolo[4,3-c]pyridin-3-yl)-3- |
| | fluoropyrrolidine-3-carbonitrile (Preparation 280): RCl-2 |
| | Prep-TLC: (50% EtOAc/PE), white solid (16 mg); LCMS m/z = |
| | 408.0 [M + H]⁺. |
| 306 | 1-(6-chloro-1-(4-(1,1-difluoroethyl)pyrimidin-2-yl)-1H- |
| | pyrazolo[4,3-c]pyridin-3-yl)-3-methylpyrrolidine-3-carbonitrile |
| | 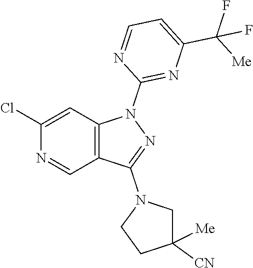 [SMILES: CC1(C#N)CCN(c2nn(-c3nccc(C(C)(F)F)n3)c3cc(Cl)ncc23)C1] |
| | SM: 1-(6-chloro-1H-pyrazolo[4,3-c]pyridin-3-yl)-3- |
| | methylpyrrolidine-3-carbonitrile (Preparation 281); RCl-1 |
| | SiO₂ (25-100% EtOAc/PE), yellow solid (95 mg, 88%); ¹H NMR |
| | (400 MHz, CDCl₃) δ: 8.97 (d, 1H), 8.87 (s, 1H), 8.57 (s, 1H), 7.41 |
| | (d, 1H), 4.30 (d, 1H), 4.14-4.03 (m, 2H), 3.74 (d, 1H), 2.66-2.62 (m, |
| | 1H), 2.22-2.04 (m, 4H), 1.65 (s, 3H). |
| 307 | 1-(6-chloro-1-(2-(1,1-difluoroethyl)pyrimidin-4-yl)-1H- |
| | pyrazolo[4,3-c]pyridin-3-yl)-3-methylpyrrolidine-3-carbonitrile |
| | 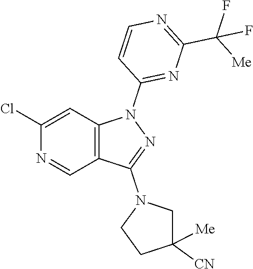 [SMILES: CC1(C#N)CCN(c2nn(-c3ccnc(C(C)(F)F)n3)c3cc(Cl)ncc23)C1] |
| | SM: 1-(6-chloro-1H-pyrazolo[4,3-c]pyridin-3-yl)-3- |
| | methylpyrrolidine-3-carbonitrile (Preparation 281); RCl-2 |
| | SiO₂ (25-100% EtOAc/PE), yellow solid (76 mg, 70%); ¹H NMR |
| | (400 MHz, CDCl₃) δ: 8.84 (s, 1H), 8.77 (d, 1H), 8.73 (s, 1H), 7.78 |
| | (d, 1H), 4.22 (d, 1H), 4.09-3.99 (m, 2H), 3.66 (d, 1H), 2.65-2.62 (m, |
| | 1H), 2.23-2.04 (m, 4H), 1.67 (s, 3H). |
| 308^{A} | 3-(azetidin-1-yl)-6-chloro-1-(4-(1,1-difluoroethyl)pyrimidin-2-yl)- |
| | 1H-pyrazolo[4,3-c]pyridine |
| | 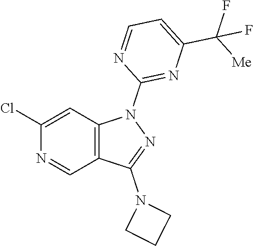 [SMILES: CC(F)(F)c1ccnc(-n2nc(N3CCC3)c3cnc(Cl)cc32)n1] |
| | SM: 3-(azetidin-1-yl)-6-chloro-1H-pyrazolo[4,3-c]pyridine |
| | (Preparation 228); RCl-1 |
| | SiO₂ (50% EtOAc/PE), white solid (72.5 mg, 43%); ¹H NMR (500 MHz, |
| | CDCl₃) δ: 8.95 (d, 1H), 8.67 (s, 1H), 8.49 (s, 1H), 7.37 (d, |
| | 1H), 4.44-4.41 (m, 4H), 2.62-2.56 (m, 2H), 2.11-2.07 (m, 3H). |
| 309^{A} | 6-chloro-3-(3,3-difluoroazetidin-1-yl)-1-(4-(1,1- |
| | difluoroethyl)pyrimidin-2-yl)-1H-pyrazolo[4,3-c]pyridine |
| | 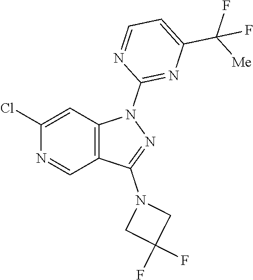 [SMILES: CC(F)(F)c1ccnc(-n2nc(N3CC(F)(F)C3)c3cnc(Cl)cc32)n1] |
| | SM: 6-chloro-3-(3,3-difluoroazetidin-1-yl)-1H-pyrazolo[4,3- |
| | c]pyridine (Preparation 263); RCl-1 |
| | SiO₂ (10-50% EtOAc/PE), yellow solid (45 mg, 25%); ¹H NMR |
| | (500 MHz, CDCl₃) δ: 8.55 (s, 1H), 7.36 (s, 1H), 5.46-5.43 (m, 1H), |
| | 4.61-4.49 (m, 4H), 4.08-4.02 (m, 1H), 3.74-3.68 (m, 1H), 2.45-2.35 |
| | (m, 1H), 2.14-2.06 (m, 1H), 2.02-1.94 (m, 1H), 1.79-1.67 (m, 3H). |
| 310 | 6-chloro-3-(3,3-difluoroazetidin-1-yl)-1-(2-(1,1- |
| | difluoroethyl)pyrimidin-4-yl)-1H-pyrazolo[4,3-c]pyridine |
| | 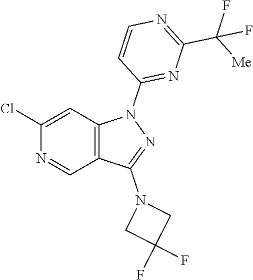 [SMILES: CC(F)(F)c1nccc(-n2nc(N3CC(F)(F)C3)c3cnc(Cl)cc32)n1] |
| | SM: 6-chloro-3-(3,3-difluoroazetidin-1-yl)-1H-pyrazolo[4,3- |
| | c]pyridine (Preparation 263); RCl-2 |
| | SiO₂ (5-33% EtOAc/PE), yellow solid (92 mg, 65%); ¹H NMR (500 MHz, |
| | CDCl₃) δ: 8.80 (d, 1H), 8.70 (d, 1H), 8.64 (s, 1H), 7.79 (d, |
| | 1H), 4.68 (t, 4H), 2.14 (t, 3H). |
| 311^{A} | 6-chloro-1-(4-(1,1-difluoroethyl)pyrimidin-2-yl)-3-(3- |
| | fluoroazetidin-1-yl)-1H-pyrazolo[4,3-c]pyridine |
| | 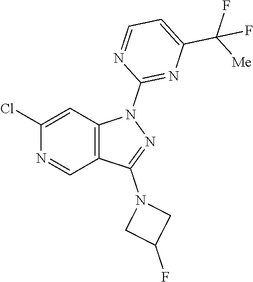 [SMILES: CC(F)(F)c1ccnc(-n2nc(N3CC(F)C3)c3cnc(Cl)cc32)n1] |
| | SM: 6-chloro-3-(3-fluoroazetidin-1-yl)-1H-pyrazolo[4,3-c]pyridine |
| | (Preparation 264); RCl-1 |
| | SiO₂ (5-33% EtOAc/PE), white solid (112 mg, 70%); ¹H NMR (500 MHz, |
| | CDCl₃) δ: 8.97 (d, 1H), 8.66 (s, 1H), 8.52 (s, 1H), 7.41 (d, |
| | 1H), 5.70-5.43 (m, 1H), 4.80-4.45 (m, 2H), 4.60-4.45 (m, 2H), 2.11 |
| | (t, 3H). |
| 312 ^{A} | 6-chloro-1-(2-(1,1-difluoroethyl)pyrimidin-4-yl)-3-(3- |
| | fluoroazetidin-1-yl)-1H-pyrazolo[4,3-c]pyridine |
| | 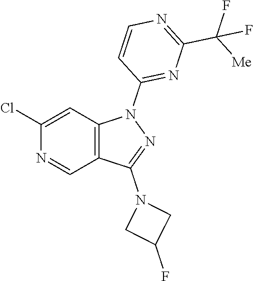 [SMILES: CC(F)(F)c1nccc(-n2nc(N3CC(F)C3)c3cnc(Cl)cc32)n1] |
| | SM: 6-chloro-3-(3-fluoroazetidin-1-yl)-1H-pyrazolo[4,3-c]pyridine |
| | (Preparation 264); RCl-2 |
| | SiO₂ (5-33% EtOAc/PE), white solid (59 mg, 60%); ¹H NMR (500 MHz, |
| | CDCl₃) δ: 8.77 (d, 1H), 8.67 (s, 1H), 8.64 (s, 1H), 7.78 (d, |
| | 1H), 5.64-5.48 (m, 1H), 4.68-4.57 (m, 2H), 4.49-4.41 (m, 2H), 2.13 |
| | (t, 3H). |
| 313 | 6-chloro-1-(4-(1,1-difluoroethyl)pyrimidin-2-yl)-3-(2- |
| | methylazetidin-1-yl)-1H-pyrazolo[4,3-c]pyridine |
| | 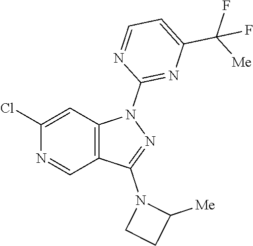 [SMILES: CC1CCN1c1nn(-c2nccc(C(C)(F)F)n2)c2cc(Cl)ncc12] |
| | SM: 6-chloro-3-(2-methylazetidin-1-yl)-1H-pyrazolo[4,3-c]pyridine |
| | (Preparation 265) |
| | SiO₂ (0-33% EtOAc/PE), white solid (37 mg, 75%); ¹H NMR (400 MHz, |
| | MeOH-d₄) δ: 8.99 (d, 1H), 8.75 (s, 1H), 8.56 (d, 1H), 7.53 (d, |
| | 1H), 4.78-4.73 (m, 1H), 4.43-4.39 (m, 1H), 4.24-4.19 (m, 1H), 2.71- |
| | 2.62 (m, 1H), 2.26-2.20 (m, 1H), 2.10 (t, 3H), 1.62 (d, 3H) |
| 314 | 6-chloro-1-(4-(1,1-difluoroethyl)pyrimidin-2-yl)-3-(3- |
| | methoxyazetidin-1-yl)-1H-pyrazolo[4,3-c]pyridine |
| | 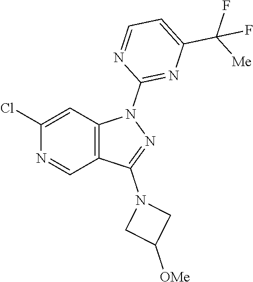 [SMILES: COC1CN(c2nn(-c3nccc(C(C)(F)F)n3)c3cc(Cl)ncc23)C1] |
| | SM: 6-chloro-3-(3-methoxyazetidin-1-yl)-1H-pyrazolo[4,3- |
| | c]pyridine (Preparation 266); RCl-1 |
| | SiO₂ (6-50% EtOAc/PE), white solid (70 mg, 63%); LCMS m/z = |
| | 381.2 [M + H]⁺. |
| 315 | 6-chloro-1-(2-(1,1-difluoroethyl)pyrimidin-4-yl)-3-(3- |
| | methoxyazetidin-1-yl)-1H-pyrazolo[4,3-c]pyridine |
| | 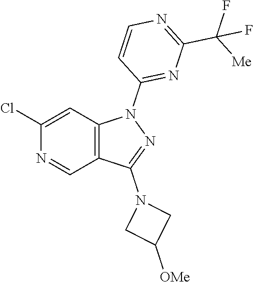 [SMILES: COC1CN(c2nn(-c3ccnc(C(C)(F)F)n3)c3cc(Cl)ncc23)C1] |
| | SM: 6-chloro-3-(3-methoxyazetidin-1-yl)-1H-pyrazolo[4,3- |
| | c]pyridine (Preparation 266); RCl-2 |
| | SiO₂ (6-50% EtOAc/PE), white solid (75 mg, 47%); LCMS m/z = |
| | 381.2 [M + H]⁺. |
| 316 | 1-(6-chloro-1-(4-(1,1-difluoroethyl)pyrimidin-2-yl)-1H- |
| | pyrazolo[4,3-c]pyridin-3-yl)azetidin-3-ol |
| | 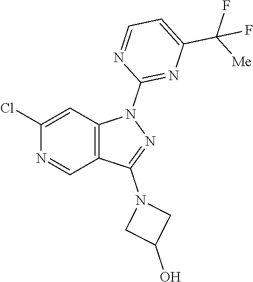 [SMILES: CC(F)(F)c1ccnc(-n2nc(N3CC(O)C3)c3cnc(Cl)cc32)n1] |
| | SM: 1-(6-chloro-1H-pyrazolo[4,3-c]pyridin-3-yl)azetidin-3-ol |
| | (Preparation 267); RCl-1 |
| | Prep-TLC (100% EtOAc), yellow solid (30 mg, 46%); LCMS m/z = |
| | 367.0 [M + H]⁺. |
| 317 | 1-(6-chloro-1-(2-(1,1-difluoroethyl)pyrimidin-4-yl)-1H- |
| | pyrazolo[4,3-c]pyridin-3-yl)azetidin-3-ol |
| | 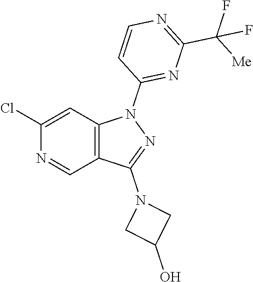 [SMILES: CC(F)(F)c1nccc(-n2nc(N3CC(O)C3)c3cnc(Cl)cc32)n1] |
| | SM: 1-(6-chloro-1H-pyrazolo[4,3-c]pyridin-3-yl)azetidin-3-ol |
| | (Preparation 267); RCl-2 |
| | SiO₂ (0-25% EtOAc/PE), yellow oil (120 mg, 61%); LCMS m/z = |
| | 367.0 [M + H]⁺. |
| 318 | 1-(6-chloro-1-(4-(1,1-difluoroethyl)pyrimidin-2-yl)-1H- |
| | pyrazolo[4,3-c]pyridin-3-yl)-3-methylazetidin-3-ol |
| | 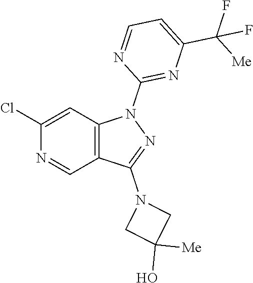 [SMILES: CC1(O)CN(c2nn(-c3nccc(C(C)(F)F)n3)c3cc(Cl)ncc23)C1] |
| | SM: 1-(6-chloro-1H-pyrazolo[4,3-c]pyridin-3-yl)-3-methylazetidin- |
| | 3-ol (Preparation 268); RCl-1 |
| | SiO₂ (6-50% EtOAc/PE), colourless oil (60 mg, 75%); LCMS m/z = |
| | 381.1 [M + H]⁺. |
| 319 | 1-(6-chloro-1-(2-(1,1-difluoroethyl)pyrimidin-4-yl)-1H- |
| | pyrazolo[4,3-c]pyridin-3-yl)-3-methylazetidin-3-ol |
| | 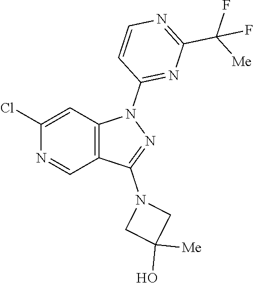 [SMILES: CC1(O)CN(c2nn(-c3ccnc(C(C)(F)F)n3)c3cc(Cl)ncc23)C1] |
| | SM: 1-(6-chloro-1H-pyrazolo[4,3-c]pyridin-3-yl)-3-methylazetidin- |
| | 3-ol (Preparation 268); RCl-2 |
| | SiO₂ (9-25% EtOAc/PE), white solid (50 mg, 52%); LCMS m/z = |
| | 381.0 [M + H]⁺. |
| 320 | 6-chloro-1-(4-(1,1-difluoroethyl)pyrimidin-2-yl)-3-(3-methoxy-3- |
| | methylazetidin-1-yl)-1H-pyrazolo[4,3-c]pyridine |
| | 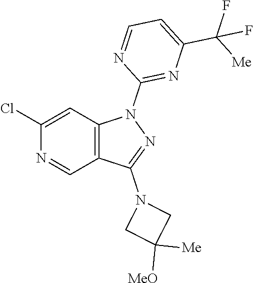 [SMILES: COC1(C)CN(c2nn(-c3nccc(C(C)(F)F)n3)c3cc(Cl)ncc23)C1] |
| | SM: 6-chloro-3-(3-methoxy-3-methylazetidin-1-yl)-1H- |
| | pyrazolo[4,3-c]pyridine (Preparation 269); RCl-1 |
| | SiO₂ (6-25% EtOAc/PE), yellow oil (70 mg, 64%); ¹H NMR (400 MHz, |
| | CDCl₃) δ: 8.90 (d, 1H), 8.61 (s, 1H), 8.44 (s, 1H), 7.33 (d, |
| | 1H), 4.30 (d, 2H), 4.11 (d, 2H), 3.26 (s, 3H), 2.04 (t, 3H), 1.56 (s, |
| | 3H). |
| 321 | 6-chloro-1-(2-(1,1-difluoroethyl)pyrimidin-4-yl)-3-(3-methoxy-3- |
| | methylazetidin-1-yl)-1H-pyrazolo[4,3-c]pyridine |
| | 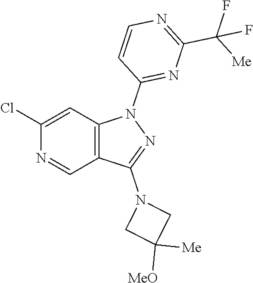 [SMILES: COC1(C)CN(c2nn(-c3ccnc(C(C)(F)F)n3)c3cc(Cl)ncc23)C1] |
| | SM: 6-chloro-3-(3-methoxy-3-methylazetidin-1-yl)-1H- |
| | pyrazolo[4,3-c]pyridine (Preparation 269); RCl-2 |
| | SiO₂ (0-50% EtOAc/PE), white solid (51 mg, 22%); ¹H NMR (500 MHz, |
| | MeOH-d₄) δ: 8.80 (d, 1H), 8.75 (s, 1H), 8.64 (s, 1H), 7.83 (d, |
| | 1H), 4.29 (d, 2H), 4.15 (d, 2H), 3.34 (s, 3H), 2.14-2.06 (m, 3H), |
| | 1.63 (s, 3H). |
| 322 | 6-chloro-1-(4-(1,1-difluoroethyl)pyrimidin-2-yl)-3-(3- |
| | (trifluoromethoxy)azetidin-1-yl)-1H-pyrazolo[4,3-c]pyridine |
| | 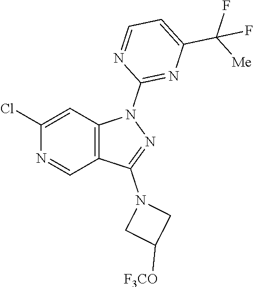 [SMILES: CC(F)(F)c1ccnc(-n2nc(N3CC(OC(F)(F)F)C3)c3cnc(Cl)cc32)n1] |
| | SM: 6-chloro-3-(3-(trifluoromethoxy)azetidin-1-yl)-1H- |
| | pyrazolo[4,3-c]pyridine (Preparation 270); RCl-1 |
| | SiO₂ (0-33% EtOAc/PE), white solid (113 mg); ¹H NMR (400 MHz, |
| | MeOH-d₄) δ: 9.01 (d, 1H), 8.78 (d, 1H), 7.56 (d, 1H), 4.79-4.74 (m, |
| | 2H), 4.49-4.46 (m, 2H), 4.13-4.07 (m, 1H), 2.10 (t, 3H). |
| 323 | 6-chloro-1-(2-(1,1-difluoroethyl)pyrimidin-4-yl)-3-(3- |
| | (trifluoromethoxy)azetidin-1-yl)-1H-pyrazolo[4,3-c]pyridine |
| | 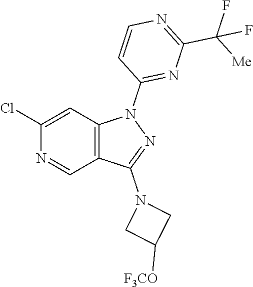 [SMILES: CC(F)(F)c1nccc(-n2nc(N3CC(OC(F)(F)F)C3)c3cnc(Cl)cc32)n1] |
| | SM: 6-chloro-3-(3-(trifluoromethoxy)azetidin-1-yl)-1H- |
| | pyrazolo[4,3-c]pyridine (Preparation 270); RCl-2 |
| | SiO₂ (0-50% EtOAc/PE), white solid (72.4 mg, 61%); ¹H NMR (400 MHz, |
| | MeOH-d₄) δ: 8.83 (d, 1H), 8.79 (s, 1H), 8.67 (d, 1H), 7.88 (d, |
| | 1H), 5.42-5.38 (m, 1H), 4.76-4.72 (m, 2H), 4.47-4.44 (m, 2H), 2.16- |
| | 2.01 (m, 3H). |
| 324^{A} | 1-(6-chloro-1-(4-(1,1-difluoroethyl)pyrimidin-2-yl)-1H- |
| | pyrazolo[4,3-c]pyridin-3-yl)-N,N-dimethylazetidin-3-amine |
| | 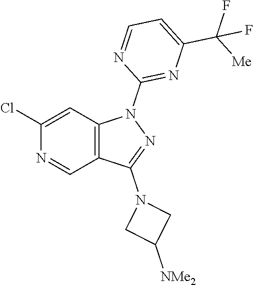 [SMILES: CN(C)C1CN(c2nn(-c3nccc(C(C)(F)F)n3)c3cc(Cl)ncc23)C1] |
| | SM: 1-(6-chloro-1H-pyrazolo[4,3-c]pyridin-3-yl)-N,N- |
| | dimethylazetidin-3-amine (Preparation 271); RCl-1 |
| | SiO₂ (0-25% EtOAc/PE), yellow solid (90 mg, 58%); LCMS m/z = |
| | 394.1 [M + H]⁺. |
| 325^{A} | 1-(6-chloro-1-(2-(1, 1-difluoroethyl)pyrimidin-4-yl)-1H- |
| | pyrazolo[4,3-c]pyridin-3-yl)-N,N-dimethylazetidin-3-amine |
| | 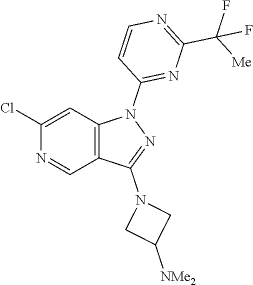 [SMILES: CN(C)C1CN(c2nn(-c3ccnc(C(C)(F)F)n3)c3cc(Cl)ncc23)C1] |
| | SM: 1-(6-chloro-1H-pyrazolo[4,3-c]pyridin-3-yl)-N,N- |
| | dimethylazetidin-3-amine (Preparation 271); RCl-2 |
| | SiO₂ (9% MeOH/DCM), white solid (65 mg, 31%); LCMS m/z = |
| | 394.2 [M + H]⁺. |
| 326^{A} | 6-(6-chloro-1-(4-(1,1-difluoroethyl)pyrimidin-2-yl)-1H- |
| | pyrazolo[4,3-c]pyridin-3-yl)-2-oxa-6-azaspiro[3.3]heptane |
| | 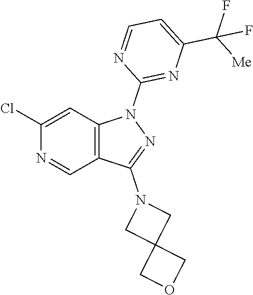 [SMILES: CC(F)(F)c1ccnc(-n2nc(N3CC4(COC4)C3)c3cnc(Cl)cc32)n1] |
| | SM: 6-(6-chloro-1H-pyrazolo[4,3-c]pyridin-3-yl)-2-oxa-6- |
| | azaspiro[3.3]heptane (Preparation 272); RCl-1 |
| | SiO₂ (50% EtOAc/PE), Yellow solid (120 mg, 96%); ¹H NMR (500 MHz, |
| | CDCl₃) δ: 8.97-8.94 (m, 1H), 8.68 (s, 1H), 8.51 (s, 1H), 7.41 |
| | (d, 1H), 4.90 (s, 4H), 4.56 (s, 4H), 2.10 (t, 3H). |
| 327^{A} | 6-(6-chloro-1-(2-(1,1-difluoroethyl)pyrimidin-4-yl)-1H- |
| | pyrazolo[4,3-c]pyridin-3-yl)-2-oxa-6-azaspiro[3.3]heptane |
| | 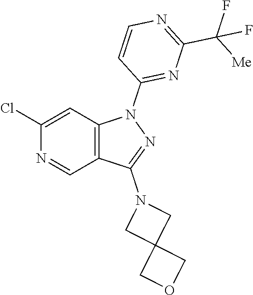 [SMILES: CC(F)(F)c1nccc(-n2nc(N3CC4(COC4)C3)c3cnc(Cl)cc32)n1] |
| | SM: 6-(6-chloro-1H-pyrazolo[4,3-c]pyridin-3-yl)-2-oxa-6- |
| | azaspiro[3.3]heptane (Preparation 272); RCl-2 |
| | SiO₂ (50% EtOAc/PE), Yellow solid (98 mg, 69%); ¹H NMR (400 MHz, |
| | CDCl₃) δ: 8.77 (d, 1H), 8.69-8.65 (m, 2H), 7.77 (d, 1H), 4.92 |
| | (s, 4H), 4.50 (s, 4H), 2.13 (d, 3H). |
| 328 | 3-(azetidin-1-yl)-6-chloro-1-(6-(1,1-difluoroethyl)pyrimidin-4-yl)- |
| | 1H-pyrazolo[4,3-c]pyridine |
| | 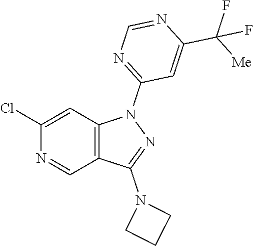 [SMILES: CC(F)(F)c1cc(-n2nc(N3CCC3)c3cnc(Cl)cc32)ncn1] |
| | SM: 3-(azetidin-1-yl)-6-chloro-1H-pyrazolo[4,3-c]pyridine |
| | (Preparation 228); RCl-6 |
| | SiO₂ (25% EtOAc/PE), white solid (143 mg, 85%); ¹H NMR (400 MHz, |
| | CDCl₃) δ: 9.05 (s, 1H), 8.67 (s, 1H), 8.64 (s, 1H), 8.02 (s, |
| | 1H), 4.38-4.34 (m, 4H), 2.65-2.57 (m, 2H), 2.03 (t, 3H). |
| 329 | 4-(6-chloro-1-(6-(1,1-difluoroethyl)pyrimidin-4-yl)-1H- |
| | pyrazolo[4,3-c]pyridin-3-yl)morpholine |
| | 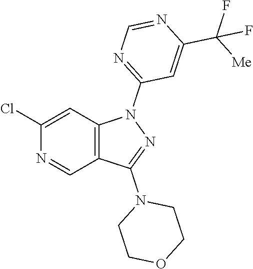 [SMILES: CC(F)(F)c1cc(-n2nc(N3CCOCC3)c3cnc(Cl)cc32)ncn1] |
| | SM: 4-(6-chloro-1H-pyrazolo[4,3-c]pyridin-3-yl)morpholine |
| | (Preparation 17); RCl-6 |
| | SiO₂ (50% EtOAc/PE), white solid (175 mg, 84%); ¹H NMR (400 MHz, |
| | CDCl₃) δ: 9.09 (s, 1H), 8.88 (s, 1H), 8.79 (s, 1H), 8.03 (s, |
| | 1H), 3.97-3.94 (m, 4H), 3.66-3.64 (m, 4H), 2.04 (t, 3H). |
| 330 | 4-(6-chloro-1-(6-(1,1-difluoroethyl)pyrimidin-4-yl)-1H-pyrrolo[3,2- |
| | c]pyridin-3-yl)morpholine |
| | 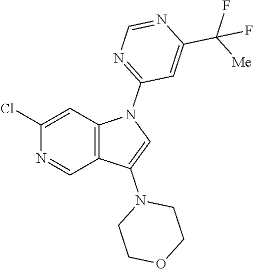 [SMILES: CC(F)(F)c1cc(-n2cc(N3CCOCC3)c3cnc(Cl)cc32)ncn1] |
| | SM: 4-(6-chloro-1H-pyrrolo[3,2-c]pyridin-3-yl)morpholine |
| | (Preparation 150); RCl-6 |
| | SiO₂ (16-50% EtOAc/PE), yellow solid (33 mg, 41%); ¹H NMR |
| | (400 MHz, CDCl₃) δ: 9.15 (s, 1H), 8.73 (s, 2H), 7.53 (s, 1H), 7.09 |
| | (s, 1H), 3.98-3.95 (m, 4H), 3.21-3.18 (m, 4H), 2.10-2.01 (m, 3H). |
| 331 | 3-(azetidin-1-yl)-6-chloro-1-(6-(1,1-difluoroethyl)-2- |
| | methylpyrimidin-4-yl)-1H-pyrazolo[4,3-c]pyridine |
| | 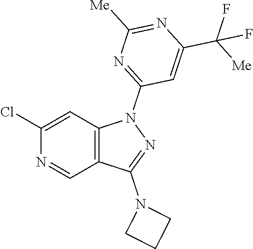 [SMILES: Cc1nc(-n2nc(N3CCC3)c3cnc(Cl)cc32)cc(C(C)(F)F)n1] |
| | SM: 3-(azetidin-1-yl)-6-chloro-1H-pyrazolo[4,3-c]pyridine |
| | (Preparation 228); RCl-4 |
| | SiO₂ (5-25% EtOAc/PE), white solid (101 mg, 72%); LCMS m/z = |
| | 365.1 [M + H]⁺; |
| 332^{A} | 4-(6-chloro-1-(6-(1,1-difluoroethyl)-2-methylpyrimidin-4-yl)-1H- |
| | pyrazolo[4,3-c]pyridin-3-yl)morpholine |
| | 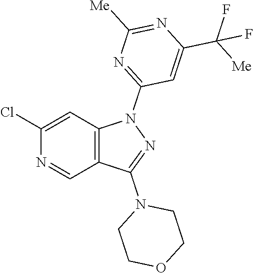 [SMILES: Cc1nc(-n2nc(N3CCOCC3)c3cnc(Cl)cc32)cc(C(C)(F)F)n1] |
| | SM: 4-(6-chloro-1H-pyrazolo[4,3-c]pyridin-3-yl)morpholine |
| | (Preparation 17); RCl-4 |
| | SiO₂ (0-33% EtOAc/PE), yellow solid (101 mg, 78%); LCMS m/z = |
| | 395.1 [M + H]⁺; |
| 333^{A} | 6-chloro-1-(2-(1,1-difluoroethyl)-6-methylpyrimidin-4-yl)-3-(4- |
| | methylpiperazin-1-yl)-1H-pyrazolo[4,3-c]pyridine |
| | 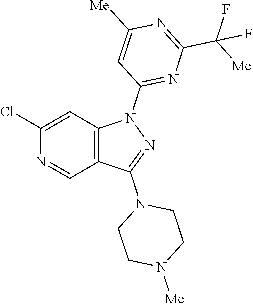 [SMILES: Cc1cc(-n2nc(N3CCN(C)CC3)c3cnc(Cl)cc32)nc(C(C)(F)F)n1] |
| | SM: 6-chloro-3-(4-methylpiperazin-1-yl)-1H-pyrazolo[4,3- |
| | c]pyridine (Preparation 282); RCl-7 |
| | Yellow solid (86 mg, 59%); LCMS m/z = 408.2 [M + H]⁺; |
| 334^{A} | 4-(6-chloro-1-(4-(1,1-difluoroethyl)pyrimidin-2-yl)-1H- |
| | pyrazolo[4,3-c]pyridin-3-yl)morpholine |
| | 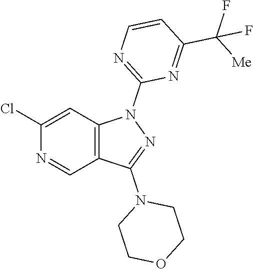 [SMILES: CC(F)(F)c1ccnc(-n2nc(N3CCOCC3)c3cnc(Cl)cc32)n1] |
| | SM: 4-(6-chloro-1H-pyrazolo[4,3-c]pyridin-3-yl)morpholine |
| | (Preparation 17); RCl-1 |
| | SiO₂ (25% EtOAc/PE), Yellow solid (480 mg, 75%); ¹H NMR (400 MHz, |
| | CDCl₃) δ: 8.98 (d, 1H), 8.90 (s, 1H), 8.59 (s, 1H), 7.43 (d, |
| | 1H), 3.96-3.91 (m, 4H), 3.69-3.67 (m, 4H), 2.11 (t, 3H). |
| 335 | 2-(6-chloro-1-(2-(1,1-difluoroethyl)pyrimidin-4-yl)-1H- |
| | pyrazolo[4,3-c]pyridin-3-yl)-5-methyl-2,5-diazabicyclo[2.2.2]octane |
| | 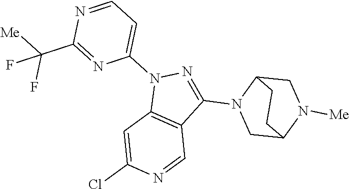 [SMILES: CN1CC2CCC1CN2c1nn(-c2ccnc(C(C)(F)F)n2)c2cc(Cl)ncc12] |
| | SM: 2-(6-chloro-1H-pyrazolo[4,3-c]pyridin-3-yl)-5-methyl-2,5- |
| | diazabicyclo[2.2.2]octane (Preparation 284); RCl-2 |
| | Brown solid (60.1 mg, 72%); LCMS m/z = 420.0 [M + H]⁺ |
| 336^{A} | 1-(6-chloro-1-(6-chloro-2-(1,1-difluoroethyl)pyrimidin-4-yl)-1H- |
| | pyrazolo[4,3-c]pyridin-3-yl)-N,N-dimethylpyrrolidin-3-amine |
| | 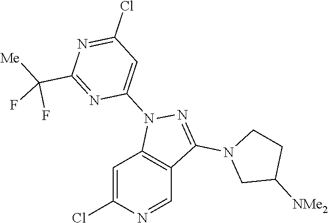 [SMILES: CN(C)C1CCN(c2nn(-c3cc(Cl)nc(C(C)(F)F)n3)c3cc(Cl)ncc23)C1] |
| | SM: 1-(6-chloro-1H-pyrazolo[4,3-c]pyridin-3-yl)-N,N- |
| | dimethylpyrrolidin-3-amine (Preparation 285); RCl-5 |
| | SiO₂ (0-10% MeOH/DCM), White solid (100 mg, 40%); ¹H NMR |
| | (500 MHz, CDCl₃) δ: 8.86 (s, 1H), 8.64 (s, 1H), 7.80 (s, 1H), 3.98- |
| | 3.93 (m, 2H), 3.79-3.76 (m, 1H), 3.60-3.58 (m, 1H), 3.50-3.48 (m, |
| | 1H), 3.01-2.98 (m, 1H), 2.39 (s, 6H), 2.35-2.30 (m, 1H), 2.12 (t, J= |
| | 18.5 Hz, 3H). |
| 337 | 1-(6-chloro-1-(2-(1,1-difluoroethyl)-6-methylpyrimidin-4-yl)-1H- |
| | pyrazolo[4,3-c]pyridin-3-yl)-N,N-dimethylpyrrolidin-3-amine |
| | 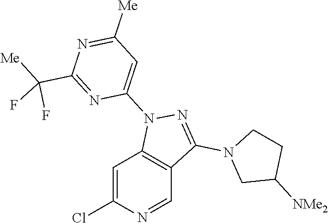 [SMILES: Cc1cc(-n2nc(N3CCC(N(C)C)C3)c3cnc(Cl)cc32)nc(C(C)(F)F)n1] |
| | SM: 1-(6-chloro-1H-pyrazolo[4,3-c]pyridin-3-yl)-N,N- |
| | dimethylpyrrolidin-3-amine (Preparation 285); RCl-7 |
| | SiO₂ (25/1 DCM/MeOH), Yellow solid (68 mg, 43%); ¹H NMR |
| | (500 MHz, MeOH-d₄) δ: 8.97 (s, 1H), 8.71 (d, 1H), 7.75 (s, 1H), |
| | 4.07-3.99 (m, 2H), 3.82-3.80 (m, 1H), 3.61-3.57 (m, 1H), 3.17-3.15 |
| | (m, 1H), 2.62 (s, 3H), 2.46 (s, 6H), 2.11 (t, 3H), 2.03-1.98 (m, 2 H). |
| 338 | 1-(6-chloro-1-(4-(1,1-difluoroethyl)pyrimidin-2-yl)-1H- |
| | pyrazolo[4,3-c]pyridin-3-yl)-N,N-dimethylpyrrolidin-3-amine |
| | 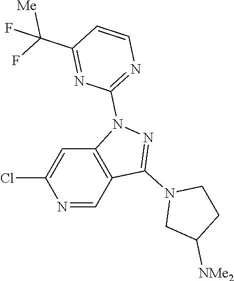 [SMILES: CN(C)C1CCN(c2nn(-c3nccc(C(C)(F)F)n3)c3cc(Cl)ncc23)C1] |
| | SM: 1-(6-chloro-1H-pyrazolo[4,3-c]pyridin-3-yl)-N,N- |
| | dimethylpyrrolidin-3-amine (Preparation 285); RCl-1 |
| | SiO₂ (0-10% MeOH/DCM), Yellow solid (99 mg, 65%); ¹H NMR |
| | (400 MHz, CDCl₃) δ: 8.96 (d, 1H), 8.89 (s, 1H), 8.54 (s, 1H), 7.37 |
| | (d, 1H), 4.07-4.02 (m, 2H), 3.84-3.82 (m, 1H), 3.64-3.60 (m, 1H), |
| | 2.97-2.94 (m, 1H), 2.37 (s, 6H), 2.37-2.31 (m, 1H), 2.14-2.04 (m, |
| | 4H). |
| 339^{A} | 1-(6-chloro-1-(4-(1,1-difluoroethyl)-6-methylpyrimidin-2-yl)-1H- |
| | pyrazolo[4,3-c]pyridin-3-yl)-N,N-dimethylpyrrolidin-3-amine |
| | 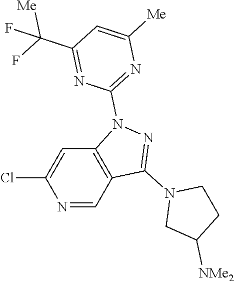 [SMILES: Cc1cc(C(C)(F)F)nc(-n2nc(N3CCC(N(C)C)C3)c3cnc(Cl)cc32)n1] |
| | SM: 1-(6-chloro-1H-pyrazolo[4,3-c]pyridin-3-yl)-N,N- |
| | dimethylpyrrolidin-3-amine (Preparation 285); RCl-3 |
| | HPLC-C (40-70% MeCN). |
| | Grey solid (60 mg, 19%); 1H NMR (400 MHz, CDCl₃) δ: 8.88 (s, |
| | 1H), 8.54 (s, 1H), 7.25 (s, 1H), 4.10-4.05 (m, 2H), 4.03-4.01 (m, |
| | 1H), 3.63-3.62 (m, 1H), 2.96-2.95 (m, 1H), 2.72 (s, 3H), 2.38-2.32 |
| | (m, 7H), 2.13-2.05 (m, 4H). |
| 340^{A} | 3-(6-chloro-1-(2-(1,1-difluoroethyl)pyrimidin-4-yl)-1H- |
| | pyrazolo[4,3-c]pyridin-3-yl)-6-methyl-3,6- |
| | diazabicyclo[3.1.1]heptane |
| | 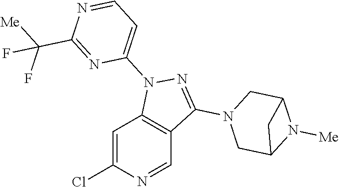 [SMILES: CN1C2CC1CN(c1nn(-c3ccnc(C(C)(F)F)n3)c3cc(Cl)ncc13)C2] |
| | SM: 3-(6-chloro-1H-pyrazolo[4,3-c]pyridin-3-yl)-6-methyl-3,6- |
| | diazabicyclo[3.1.1]heptane (Preparation 288); RCl-2 |
| | SiO₂ (100% EtOAc), Yellow solid (25 mg, 36%). LCMS m/z = |
| | 406.0 [M + H]⁺ |
| 341^{A} | 1-(6-chloro-1-(2-(1,1-difluoroethyl)pyrimidin-4-yl)-1H- |
| | pyrazolo[4,3-c]pyridin-3-yl)-6-oxa-1-azaspiro[3.3]heptane |
| | 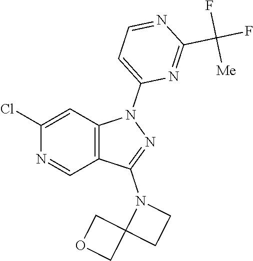 [SMILES: CC(F)(F)c1nccc(-n2nc(N3CCC34COC4)c3cnc(Cl)cc32)n1] |
| | SM: 1-(6-chloro-1H-pyrazolo[4,3-c]pyridin-3-yl)-6-oxa-1- |
| | azaspiro[3.3]heptane (Preparation 274); RCl-2 |
| | SiO₂ (0-50% EtOAc/PE), Yellow solid (40 mg, 85%). ¹H NMR (500 MHz, |
| | CDCl₃) δ: 8.84 (s, 1H), 8.77 (d, 1H), 8.71 (s, 1H), 7.87 (d, |
| | 1H), 5.57 (d, 2H), 4.79 (d, 2H), 4.26 (t, 2H), 2.84 (t, 2H), 2.14 (t, |
| | 3H). |
| 342^{A} | 6-chloro-1-(2-(1,1-difluoroethyl)pyrimidin-4-yl)-3-(3-methyl-3,6- |
| | diazabicyclo[3.2.0]heptan-6-yl)-1H-pyrazolo[4,3-c]pyridine |
| | 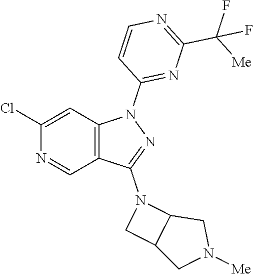 [SMILES: CN1CC2CN(c3nn(-c4ccnc(C(C)(F)F)n4)c4cc(Cl)ncc34)C2C1] |
| | SM: 6-chloro-3-(3-methyl-3,6-diazabicyclo[3.2.0]heptan-6-yl)-1H- |
| | pyrazolo[4,3-c]pyridine (Preparation 286); RCl-2 |
| | SiO₂ (10% MeOH/DCM), Yellow solid (60 mg, 78%). LCMS m/z = |
| | 407.6 [M + H]⁺ |
| 343^{A} | 6-chloro-1-(2-(1,1-difluoroethyl)pyrimidin-4-yl)-3-(1- |
| | methylhexahydropyrrolo[3,4-b]pyrrol-5(1H)-yl)-1H-pyrazolo[4,3- |
| | c]pyridine |
| | 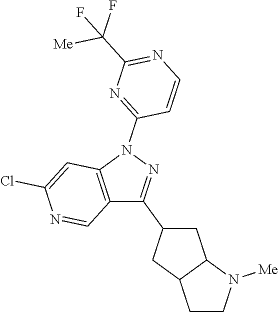 [SMILES: CN1CCC2CC(c3nn(-c4ccnc(C(C)(F)F)n4)c4cc(Cl)ncc34)CC21] |
| | SM: 6-chloro-3-(1-methylhexahydropyrrolo[3,4-b]pyrrol-5(1H)-yl)- |
| | 1H-pyrazolo[4,3-c]pyridine (Preparation 287); RCl-2 |
| | SiO₂ (10% MeOH/DCM), Yellow solid (20 mg, 33%). ¹H NMR |
| | (500 MHz, MeOH-d₄) δ: 9.02 (s, 1H), 8.80 (d, 1H), 8.72 (s, 1H), |
| | 7.89 (d, 1H), 3.94-3.90 (m, 2H) 3.71-3.67 (m, 2H), 3.17-3.12 (m, |
| | 3H), 2.48-2.44 (m, 3H), 2.25-2.22 (m, 1H), 2.24-2.10 (m, 3H), 1.83- |
| | 1.79 (m, 1H), 0.91-0.87 (m, 1H). |
| 344^{A} | 1-(6-chloro-1-(2-(1,1-difluoroethyl)pyrimidin-4-yl)-1H- |
| | pyrazolo[4,3-c]pyridin-3-yl)-N,N,3-trimethylpyrrolidin-3-amine |
| | 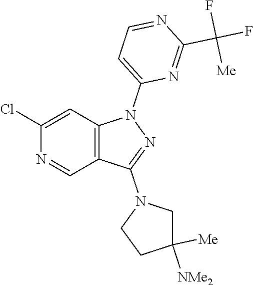 [SMILES: CN(C)C1(C)CCN(c2nn(-c3ccnc(C(C)(F)F)n3)c3cc(Cl)ncc23)C1] |
| | SM: 1-(6-chloro-1H-pyrazolo[4,3-c]pyridin-3-yl)-N,N,3- |
| | trimethylpyrrolidin-3-amine (Preparation 275); RCl-2 |
| | SiO₂ (10% MeOH/DCM), Yellow solid (34 mg, 32%). ¹H NMR |
| | (500 MHz, MeOH-d₄) δ: 8.98 (s, 1H), 8.80 (d, 1H), 8.72 (s, 1H), |
| | 7.88 (d, 1H), 4.01-3.98 (m, 1H), 3.90-3.88 (m, 1H), 3.79-3.77 (m, |
| | 1H), 3.67-3.65 (m, 1H), 2.40 (s, 6H), 2.13-2.07 (m, 3H), 1.35-1.29 |
| | (m, 2H), 1.24 (s, 3H). |
| 345 | 6-chloro-1-(2-(1,1-difluoroethyl)pyrimidin-4-yl)-3-(5- |
| | methylhexahydropyrrolo[3,4-c]pyrrol-2(1H)-yl)-1H-pyrazolo[4,3- |
| | c]pyridine |
| | 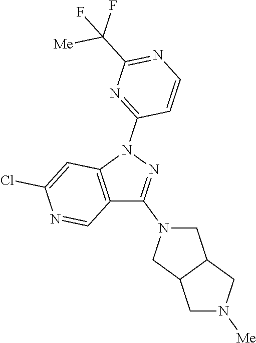 [SMILES: CN1CC2CN(c3nn(-c4ccnc(C(C)(F)F)n4)c4cc(Cl)ncc34)CC2C1] |
| | SM: 6-chloro-3-(5-methylhexahydropyrrolo[3,4-c]pyrrol-2(1H)-yl)- |
| | 1H-pyrazolo[4,3-c]pyridine (Preparation 289); RCl-2 |
| | White solid (88 mg, crude). LCMS m/z = 420.1 [M + H]⁺. |
| 346^{A} | 6-chloro-1-(2-(1,1-difluoroethyl)pyrimidin-4-yl)-3-(8-methyl-3,8- |
| | diazabicyclo[3.2.1]octan-3-yl)-1H-pyrazolo[4,3-c]pyridine |
| | 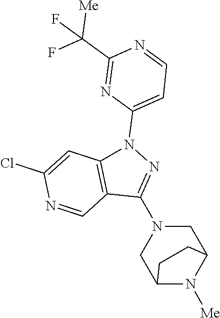 [SMILES: CN1C2CCC1CN(c1nn(-c3ccnc(C(C)(F)F)n3)c3cc(Cl)ncc13)C2] |
| | SM: 6-chloro-3-(8-methyl-3,8-diazabicyclo[3.2.1]octan-3-yl)-1H- |
| | pyrazolo[4,3-c]pyridine (Preparation 290); RCl-2 |
| | HPLC-C (45-75% MeCN). White solid (90 mg, 33). ¹H NMR (500 MHz, |
| | CDCl₃) δ: 8.88 (s, 1H), 8.76-8.74 (m, 2H), 7.78 (d, 1H), 3.82- |
| | 3.80 (m, 2H), 3.50-3.48 (m, 2H), 3.34-3.33 (m, 2H), 2.40 (s, 3H), |
| | 2.17-2.09 (m, 5H), 1.86-1.84 (m, 2H). |
| 347 | 6-chloro-1-(2-(1,1-difluoroethyl)pyrimidin-4-yl)-3-(3-methyl-3,8- |
| | diazabicyclo[3.2.1]octan-8-yl)-1H-pyrazolo[4,3-c]pyridine |
| | 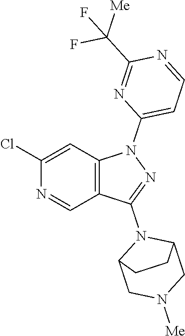 [SMILES: CN1CC2CCC(C1)N2c1nn(-c2ccnc(C(C)(F)F)n2)c2cc(Cl)ncc12] |
| | SM: 6-chloro-3-(3-methyl-3,8-diazabicyclo[3.2.1]octan-8-yl)-1H- |
| | pyrazolo[4,3-c]pyridine (Preparation 291); RCl-2 |
| | White solid (100 mg, crude). LCMS m/z = 420.1 [M + H]⁺. |
| 348^{A} | 6-chloro-1-(2-(1,1-difluoroethyl)pyrimidin-4-yl)-3-(5-methyl-2,5- |
| | diazabicyclo[2.2.1]heptan-2-yl)-1H-pyrazolo[4,3-c]pyridine |
| | 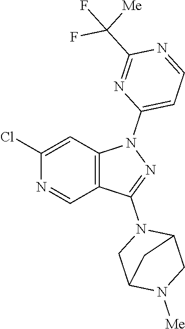 [SMILES: CN1CC2CC1CN2c1nn(-c2ccnc(C(C)(F)F)n2)c2cc(Cl)ncc12] |
| | SM: 6-chloro-3-(5-methyl-2,5-diazabicyclo[2.2.1]heptan-2-yl)-1H- |
| | pyrazolo[4,3-c]pyridine (Preparation 292); RCl-2 |
| | White solid (50 mg, 32%). ¹H NMR (500 MHz, CDCl₃) δ: 8.80 (s, |
| | 1H), 8.74 (d, 1H), 8.70 (s, 1H), 7.78 (d, 1H), 4.62 (s, 1H), 3.89-3.74 |
| | (m, 1H), 3.74-3.72 (m, 1H), 3.61-3.59 (m, 1H), 3.00-2.98 (m, 1H), |
| | 2.89-2.87 (m, 1H), 2.44 (s, 3H), 2.17-2.10 (m, 4H), 2.07-1.95 (m, |
| | 1H). |
| 349 | 6-(6-chloro-1-(2-(1,1-difluoroethyl)pyrimidin-4-yl)-1H- |
| | pyrazolo[4,3-c]pyridin-3-yl)-3,3-difluoro-1-oxa-6- |
| | azaspiro[3.3]heptane |
| | 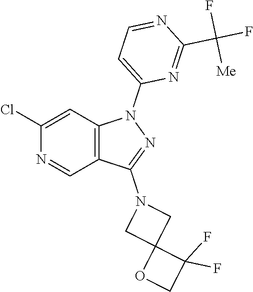 [SMILES: CC(F)(F)c1nccc(-n2nc(N3CC4(C3)OCC4(F)F)c3cnc(Cl)cc32)n1] |
| | SM: 6-(6-chloro-1H-pyrazolo[4,3-c]pyridin-3-yl)-3,3-difluoro-1- |
| | oxa-6-azaspiro[3.3]heptane (Preparation 276); RCl-2 |
| | White solid (30 mg, 50%). LCMS m/z = 429.0 [M + H]⁺ |

A-DMF was the reaction solvent

### Preparation 350: 1-(6-chloro-1-(4-(1,1-difluoroethyl)-6-methoxypyrimidin-2-yl)-1H-pyrazolo[4,3-c]pyridin-3-yl)-N,N-dimethylpyrrolidin-3-amine and 1-(6-chloro-1-(6-(1,1-difluoroethyl)-2-methoxypyrimidin-4-yl)-1H-pyrazolo[4,3-c]pyridin-3-yl)-N,N-dimethylpyrrolidin-3-amine

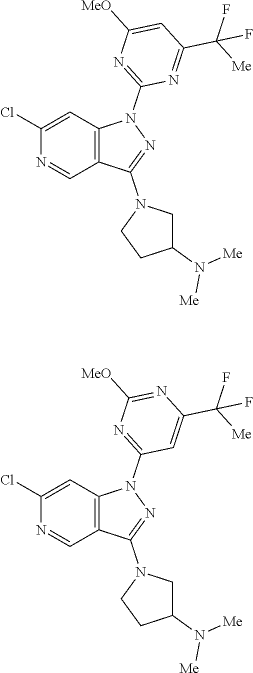 [SMILES: COc1cc(C(C)(F)F)nc(-n2nc(N3CCC(N(C)C)C3)c3cnc(Cl)cc32)n1 ; COc1nc(-n2nc(N3CCC(N(C)C)C3)c3cnc(Cl)cc32)cc(C(C)(F)F)n1]

To a solution of 2-chloro-4-(1,1-difluoroethyl)-6-methoxypyrimidine and 4-chloro-6-(1,1-difluoroethyl)-2-methoxypyrimidine (Preparation 219, 200 mg, 0.959 mmol) in DMF (5 mL) was added 1-(6-chloro-1H-pyrazolo[4,3-c]pyridin-3-yl)-N,N-dimethylpyrrolidin-3-amine (Preparation 285, 254.8 mg, 0.959 mmol) and Cs₂CO₃ (624.8 mg, 1.92 mmol) and the reaction stirred at 100° C. for 16 h. The cooled mixture was concentrated and the residue was purified by chromatography on silica gel (EtOAc) to give 1-(6-chloro-1-(4-(1,1-difluoroethyl)-6-methoxypyrimidin-2-yl)-1H-pyrazolo[4,3-c]pyridin-3-yl)-N,N-dimethylpyrrolidin-3-amine and 1-(6-chloro-1-(6-(1,1-difluoroethyl)-2-methoxypyrimidin-4-yl)-1H-pyrazolo[4,3-c]pyridin-3-yl)-N,N-dimethylpyrrolidin-3-amine (90 mg, 21.4% yield) as a yellow oil. LCMS m/z=438.1 [M+H]⁺

### Preparation 351: 6-chloro-3-cyclopropyl-1-(2-(1,1-difluoroethyl)pyrimidin-4-yl)-1H-pyrrolo[3,2-c]pyridine

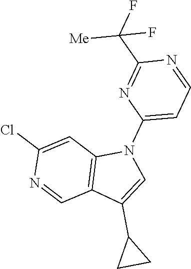 [SMILES: CC(F)(F)c1nccc(-n2cc(C3CC3)c3cnc(Cl)cc32)n1]

A mixture of 6-chloro-3-cyclopropyl-1H-pyrrolo[3,2-c]pyridine (Preparation 27, 95 mg, 0.493 mmol), 4-chloro-2-(1,1-difluoroethyl)pyrimidine (Preparation 184, 105.7 mg, 0.592 mmol) and Cs₂CO₃ (321.4 mg, 0.986 mmol) in DMSO (3 mL) was heated at 80° C. for 2 h. The cooled mixture was diluted with water, the solid formed was washed with water (3×), then dried to give 6-chloro-3-cyclopropyl-1-(2-(1,1-difluoroethyl)pyrimidin-4-yl)-1H-pyrrolo[3,2-c]pyridine (153 mg, 92% yield) as a white solid which was used in the next step without further purification. LCMS m/z=335.0 [M+H]⁺1H NMR (400 MHz, CDCl₃) δ 8.90-8.80 (m, 1H), 8.81 (s, 1H), 8.62 (s, 1H), 7.38 (d, 1H, J=0.8 Hz), 7.35 (d, 1H, J=5.8 Hz), 2.15 (t, 3H, J=18.4 Hz), 2.10-2.00 (m, 1H), 1.10-1.00 (m, 2H), 0.80-0.70 (m, 2H).

### Preparation 352: 6-chloro-3-(4-methoxypiperidin-1-yl)-1-(tetrahydro-2H-pyran-2-yl)-1H-pyrazolo[4,3-c]pyridine

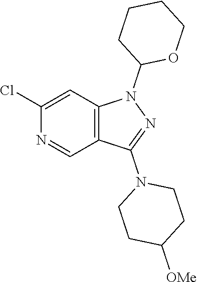 [SMILES: COC1CCN(c2nn(C3CCCCO3)c3cc(Cl)ncc23)CC1]

6-Chloro-3-(4-methoxypiperidin-1-yl)-1-(tetrahydro-2H-pyran-2-yl)-1H-pyrazolo[4,3-c]pyridine was obtained as a yellow oil, 100 mg, 34.5% yield from 6-chloro-3-iodo-1-(tetrahydro-2H-pyran-2-yl)-1H-pyrazolo[4,3-c]pyridine and 4-methoxypiperidine, following the procedure described in Preparation 229. ¹H NMR: (500 MHz, CDCl₃) δ: ppm 8.76 (s, 1H), 7.33 (s, 1H), 5.45-5.43 (m, 1H), 4.04-4.03 (m, 2H), 3.91-3.90 (m, 1H), 3.81-3.80 (m, 2H), 3.79-3.76 (m, 1H), 3.44-3.42 (m, 2H), 3.39 (s, 3H), 3.23-3.20 (m, 2H), 2.41-2.40 (m, 1H), 1.78-1.71 (m, 6H).

### Preparation 353: 6-chloro-3-(pyrrolidin-1-yl)-1-(tetrahydro-2H-pyran-2-yl)-1H-pyrazolo[4,3-c]pyridine

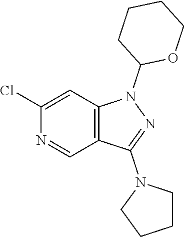 [SMILES: Clc1cc2c(cn1)c(N1CCCC1)nn2C1CCCCO1]

6-Chloro-3-(pyrrolidin-1-yl)-1-(tetrahydro-2H-pyran-2-yl)-1H-pyrazolo[4,3-c]pyridine was obtained as a yellow oil, 220 mg, 65.2% yield from 6-chloro-3-iodo-1-(tetrahydro-2H-pyran-2-yl)-1H-pyrazolo[4,3-c]pyridine and pyrrolidine, following the procedure described in Preparation 229. 1H NMR: (500 MHz, CDCl₃) δ: ppm 8.75 (s, 1H), 7.27 (s, 1H), 5.43-5.40 (m, 1H), 4.09-4.05 (m, 2H), 3.66-3.60 (m, 4H), 2.11-2.07 (m, 2H), 2.04-2.02 (m, 4H), 1.73-1.70 (m, 4H).

### Preparation 354: 6-chloro-3-(4-methoxypiperidin-1-yl)-1H-pyrazolo[4,3-c]pyridine trifluoroacetate

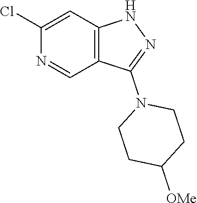 [SMILES: COC1CCN(c2n[nH]c3cc(Cl)ncc23)CC1]

A mixture of 6-chloro-3-(4-methoxypiperidin-1-yl)-1-(tetrahydro-2H-pyran-2-yl)-1H-pyrazolo[4,3-c]pyridine (Preparation 352, 100 mg, 0.285 mmol) in TFA (595.6 mg, 5.22 mmol) and DCM (2 mL) was stirred at 20° C. for 12 h. The mixture was concentrated under reduced pressure to give 6-chloro-3-(4-methoxypiperidin-1-yl)-1H-pyrazolo[4,3-c]pyridine trifluoroacetate (70 mg, 92.1% yield) as a yellow oil. LCMS m/z=267.1 [M+H]⁺

### Preparation 355: 6-chloro-3-(pyrrolidin-1-yl)-1H-pyrazolo[4,3-c]pyridine hydrochloride

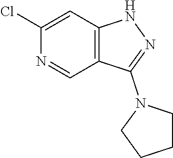 [SMILES: Clc1cc2[nH]nc(N3CCCC3)c2cn1]

A mixture of 6-chloro-3-(pyrrolidin-1-yl)-1-(tetrahydro-2H-pyran-2-yl)-1H-pyrazolo[4,3-c]pyridine (Preparation 353, 220 mg, 0.717 mmol) in HCl/dioxane (4 M, 2 mL) and DCM (2 mL) was stirred at 25° C. for 2 hours. The mixture was concentrated under reduced pressure to give 6-chloro-3-(pyrrolidin-1-yl)-1H-pyrazolo[4,3-c]pyridine hydrochloride (180 mg, 96.86% yield) as a yellow solid. LCMS m/z=223.1 [M+H]⁺

### Preparation 356: 6-chloro-1-(2-(1,1-difluoroethyl)pyrimidin-4-yl)-3-(4-methoxypiperidin-1-yl)-1H-pyrazolo[4,3-c]pyridine

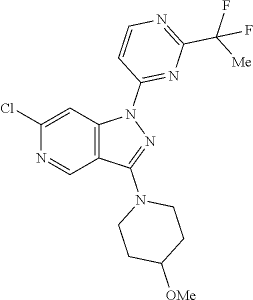 [SMILES: COC1CCN(c2nn(-c3ccnc(C(C)(F)F)n3)c3cc(Cl)ncc23)CC1]

A mixture of 6-chloro-3-(4-methoxypiperidin-1-yl)-1H-pyrazolo[4,3-c]pyridine trifluoroacetate (Preparation 354, 60 mg, 0.225 mmol), 4-chloro-2-(1,1-difluoroethyl)pyrimidine (Preparation 184, 40.2 mg, 0.225 mmol) and Cs₂CO₃ (146.6 mg, 0.450 mmol) in DMF (3 mL) was stirred at 100° C. for 12 h. The mixture was concentrated under reduced pressure and the residue was purified by chromatography on silica gel (PE/EtOAc=1/2) to give 6-chloro-1-(2-(1,1-difluoroethyl)pyrimidin-4-yl)-3-(4-methoxypiperidin-1-yl)-1H-pyrazolo[4,3-c]pyridine (50 mg, 54.4% yield) as a yellow oil. ¹H NMR: (400 MHz, CDCl₃) δ: ppm 8.89 (s, 1H), 8.77-8.74 (m, 2H), 7.79 (d, J=5.6 Hz, 1H), 3.80-3.78 (m, 1H), 3.53-3.43 (m, 4H), 3.41 (s, 3H), 2.13 (t, J=18.4 Hz, 3H), 2.00-1.95 (m, 4H).

### Preparation 357: 6-chloro-1-(2-(1,1-difluoroethyl)pyrimidin-4-yl)-3-(pyrrolidin-1-yl)-1H-pyrazolo[4,3-c]pyridine

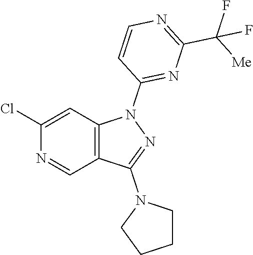 [SMILES: CC(F)(F)c1nccc(-n2nc(N3CCCC3)c3cnc(Cl)cc32)n1]

6-Chloro-1-(2-(1,1-difluoroethyl)pyrimidin-4-yl)-3-(pyrrolidin-1-yl)-1H-pyrazolo[4,3-c]pyridine was obtained as a yellow solid, 90 mg, 36.6% yield, from 6-chloro-3-(pyrrolidin-1-yl)-1H-pyrazolo[4,3-c]pyridine hydrochloride (Preparation 355) and 4-chloro-2-(1,1-difluoroethyl)pyrimidine (Preparation 184) following the procedure described in Preparation 356. 1H NMR: (400 MHz, CDCl₃) δ: ppm 8.88 (s, 1H), 8.76 (d, J=5.6 Hz, 1H), 8.72 (s, 1H), 7.79 (d, J=6.0 Hz, 1H), 3.77-3.75 (m, 4H), 2.18-2.09 (m, 7H).

### Preparation 358: 6-chloro-3-iodo-1-trityl-1H-pyrrolo[3,2-c]pyridine

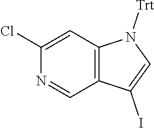

To a solution of 6-chloro-3-iodo-1H-pyrrolo[3,2-c]pyridine (1.8 g, 6.46 mmol) in DMF (20 mL) was added TrtCl (1.98 g, 7.11 mmol) and Cs₂CO₃ (4.2 g, 12.93 mmol) and the reaction was stirred at 70° C. for 16 h. The cooled mixture was concentrated and the residue was purified by chromatography on silica gel (PE/EtOAc=10/1) to give 6-chloro-3-iodo-1-trityl-1H-pyrrolo[3,2-c]pyridine (2.61 g, 77.5% yield) as a yellow solid.

### Preparation 359: 3-(azetidin-1-yl)-6-chloro-1-trityl-1H-pyrrolo[3,2-c]pyridine

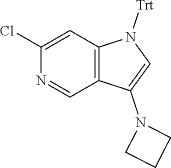

To a solution of 6-chloro-3-iodo-1-trityl-1H-pyrrolo[3,2-c]pyridine (Preparation 358, 2 g, 3.84 mmol) in DMSO (20 mL) was added azetidine hydrochloride (718.6 mg, 7.68 mmol), L-proline (88.4 mg, 0.768 mmol), K₂CO₃ (1.1 g, 7.68 mmol) and CuI (219.4 mg, 1.15 mmol) and the resulting mixture was stirred at 100° C. for 3 h under N₂. The cooled mixture was quenched with H₂O (20 mL) and extracted with EtOAc (20 mL×2). The combined organic phase was washed with brine (20 mL×2), dried over Na₂SO₄ and filtered. The filtrate was concentrated under reduced pressure and the residue was purified by chromatography on silica gel (PE/EtOAc=5/1) to give 3-(azetidin-1-yl)-6-chloro-1-trityl-1H-pyrrolo[3,2-c]pyridine (700 mg, 40.5% yield) as a black oil. ¹H NMR: (500 MHz, CDCl₃) δ: ppm 8.49 (s, 1H), 7.30-7.29 (m, 9H), 7.17-7.16 (m, 6H), 6.26-6.23 (m, 2H), 3.86-3.81 (m, 4H), 2.39-2.34 (m, 2H).

### Preparation 360: 3-(azetidin-1-yl)-6-chloro-1H-pyrrolo[3,2-c]pyridine

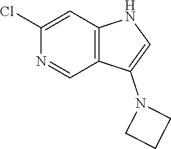 [SMILES: Clc1cc2[nH]cc(N3CCC3)c2cn1]

To a solution of 3-(azetidin-1-yl)-6-chloro-1-trityl-1H-pyrrolo[3,2-c]pyridine (Preparation 359, 540 mg, 1.20 mmol) in DCM (20 mL) was added TFA (6.0 g, 52.24 mmol) and Et₃SiH (558.2 mg, 4.80 mmol) and the reaction was stirred at 25° C. for 16 h. The mixture was quenched with H₂O (20 mL), extracted with DCM (20 mL×2), the combined organic phase was washed with brine (20 mL×2) and dried over Na₂SO₄ and filtered. The filtrate was concentrated under reduced pressure and the residue was purified by chromatography on silica gel (PE/EtOAc=1/1) to give 3-(azetidin-1-yl)-6-chloro-1H-pyrrolo[3,2-c]pyridine (45 mg, 18.1% yield) as a yellow solid. ¹H NMR: (500 MHz, CDCl₃) δ: ppm 8.57 (s, 1H), 7.20 (s, 1H), 6.42 (d, J=2.5 Hz, 1H), 4.73 (s, 1H), 3.96-3.91 (m, 4H), 2.45-2.39 (m, 2H).

### Preparation 361: 3-(azetidin-1-yl)-6-chloro-1-(6-(1,1-difluoroethyl)pyrimidin-4-yl)-1H-pyrrolo[3,2-c]pyridine

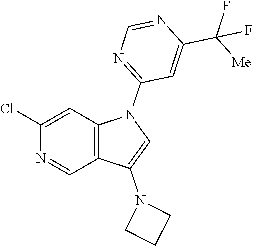 [SMILES: CC(F)(F)c1cc(-n2cc(N3CCC3)c3cnc(Cl)cc32)ncn1]

To a solution of 3-(azetidin-1-yl)-6-chloro-1H-pyrrolo[3,2-c]pyridine (Preparation 360, 45 mg, 0.217 mmol) in DMF (2 mL) was added 4-chloro-6-(1,1-difluoroethyl)pyrimidine (38.7 mg, 0.217 mmol) and Cs₂CO₃ (141.2 mg, 0.433 mmol) and the mixture was stirred at 100° C. for 3 h. The mixture was quenched with H₂O (20 mL) and extracted with EtOAc (20 mL×2). The combined organic phase was washed with brine (20 mL×2), dried over Na₂SO₄ and filtered. The filtrate was concentrated under reduced pressure and the residue was purified by chromatography on silica gel (EtOAc) to give 3-(azetidin-1-yl)-6-chloro-1-(6-(1,1-difluoroethyl)pyrimidin-4-yl)-1H-pyrrolo[3,2-c]pyridine (40 mg, 52.8% yield) as a yellow solid. ¹H NMR (500 MHz, CDCl₃) δ: 9.11 (s, 1H), 8.70 (s, 1H), 8.60 (s, 1H), 7.45 (s, 1H), 6.74 (s, 1H), 4.10-4.06 (m, 4H), 2.53-2.48 (m, 2H), 2.08-2.01 (m, 3H).

### Preparation 362: 1-(6-chloro-1-(4-(1,1-difluoroethyl)pyrimidin-2-yl)-1H-pyrazolo[4,3-c]pyridin-3-yl)-3-fluoroazetidine-3-carbonitrile

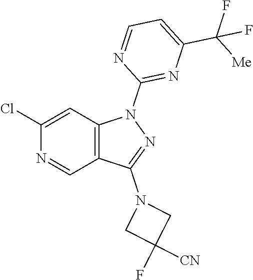 [SMILES: CC(F)(F)c1ccnc(-n2nc(N3CC(F)(C#N)C3)c3cnc(Cl)cc32)n1]

To a solution of 1-(6-chloro-1H-pyrazolo[4,3-c]pyridin-3-yl)-3-fluoroazetidine-3-carbonitrile trifluoroacetate (Preparation 273, 73.0 mg, 0.290 mmol) and 2-chloro-4-(1,1-difluoroethyl)pyrimidine (Preparation 178, 62.2 mg, 0.348 mmol) in dioxane (7.0 mL) was added K₃PO₄ (86.2 mg, 0.406 mmol), Pd₂(dba)₃ (26.6 mg, 0.029 mmol) and Xantphos (16.8 mg, 0.029 mmol) at 25° C. and the reaction was stirred at 70° C. for 16 h under N₂. The mixture was concentrated and purified by column chromatography (PE/EtOAc=5/1 to 1/1) on silica gel to give 1-(6-chloro-1-(4-(1,1-difluoroethyl)pyrimidin-2-yl)-1H-pyrazolo[4,3-c]pyridin-3-yl)-3-fluoroazetidine-3-carbonitrile (100.0 mg, 87.6% yield) as a white solid. ¹H NMR (400 MHz, CDCl₃) δ: 8.99 (d, 1H), 8.65 (s, 1H), 8.55 (s, 1H), 7.47 (d, 1H), 4.96-4.89 (m, 2H), 4.81-4.75 (m, 2H), 2.16-2.06 (m, 3H).

### Preparation 363: 1-(6-chloro-1-(2-(1,1-difluoroethyl)-6-methoxypyrimidin-4-yl)-1H-pyrazolo[4,3-c]pyridin-3-yl)-N,N-dimethylpyrrolidin-3-amine

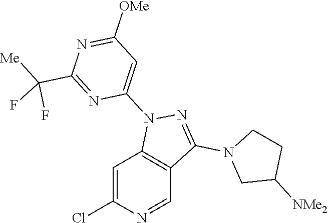 [SMILES: COc1cc(-n2nc(N3CCC(N(C)C)C3)c3cnc(Cl)cc32)nc(C(C)(F)F)n1]

A solution of 1-(6-chloro-1-(6-chloro-2-(1,1-difluoroethyl)pyrimidin-4-yl)-1H-pyrazolo[4,3-c]pyridin-3-yl)-N,N-dimethylpyrrolidin-3-amine (Preparation 336, 90 mg, 0.203 mmol) and MeONa (16.5 mg, 0.305 mmol) in MeOH (8 mL) was stirred at 25° C. for 16 h. The mixture was quenched with H₂O (50.0 mL) and extracted with EtOAc (20.0 mL×3). The combined organics were washed with brine (20 mL), dried (Na₂SO₄) and concentrated under vacuum to give 1-(6-chloro-1-(2-(1,1-difluoroethyl)-6-methoxypyrimidin-4-yl)-1H-pyrazolo[4,3-c]pyridin-3-yl)-N,N-dimethylpyrrolidin-3-amine as a white solid (40 mg, 45% yield). ¹H NMR (500 MHz, CDCl₃) δ: (s, 1H), 8.68 (s, 1H), 7.13 (s, 1H), 4.08 (s, 3H), 3.95-3.71 (m, 2H), 3.77-3.73 (m, 1H), 3.56-3.51 (m, 1H), 2.95-2.93 (m, 1H), 2.37 (s, 6H), 2.32-2.28 (m, 1H), 2.11 (t, J=18.5 Hz, 3H), 2.06-2.03 (m, 1H).

### Preparation 364: 3,6-dibromo-1-(tetrahydro-2H-pyran-2-yl)-1H-pyrazolo[4,3-c]pyridine

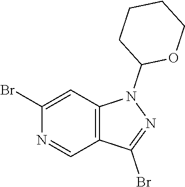 [SMILES: Brc1cc2c(cn1)c(Br)nn2C1CCCCO1]

3,6-Dibromo-1H-pyrazolo[4,3-c]pyridine (200 g, 722.2 mmol) was dissolved in EtOAc (1.0 L). DHP (121.50 g, 1.44 mol) and TFA (411.76 g, 3.61 mol) were added and the mixture was stirred at 70° C. for 12 h. The cooled reaction was poured into ice water (500 mL), then basified to pH=8.0 with saturated aq. Na₂CO₃. The mixture was partitioned between brine and EtOAc, the layers separated, the organic layer was concentrated until most of precipitate generated. The solid was collected by filtration, then slurried with PE: Et₂O (1:1, 200 mL), filtered and dried to afford 3,6-dibromo-1-(tetrahydro-2H-pyran-2-yl)-1H-pyrazolo[4,3-c]pyridine (150 g, 57.5%) as a white solid.

### Preparation 365: N-(3-bromo-1-(tetrahydro-2H-pyran-2-yl)-1H-pyrazolo[4,3-c]pyridin-6-yl)acetamide

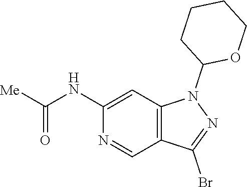 [SMILES: CC(=O)Nc1cc2c(cn1)c(Br)nn2C1CCCCO1]

A mixture of 3,6-dibromo-1-(tetrahydro-2H-pyran-2-yl)-1H-pyrazolo[4,3-c]pyridine (Preparation 364, 150 g, 415.5 mmol), AcNH₂ (34.36 g, 581.7 mmol), Pd₂(dba)₃ (11.41 g, 12.46 mmol), XantPhos (14.42 g, 24.9 mmol) and Cs₂CO₃ (203.06 g, 623.2 mmol) in DMA (800 mL), was heated to 100° C. and stirred for 3 h under N₂ atmosphere. The cooled reaction mixture was diluted with EtOAc (500 mL), filtered and the filtrate was poured into ice water (2.0 L). After stirring for 1 h, the precipitate was collected by filtration, the filter cake was washed with EtOAc (100 mL) and dried to give N-(3-bromo-1-(tetrahydro-2H-pyran-2-yl)-1H-pyrazolo[4,3-c]pyridin-6-yl)acetamide (100 g, 71.0%) as white solid. LCMS m/z=338.9 [M+H]⁺

### Preparation 366: N-(3-bromo-1H-pyrazolo[4,3-c]pyridin-6-yl)acetamide

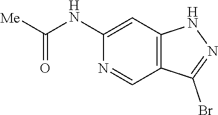 [SMILES: CC(=O)Nc1cc2[nH]nc(Br)c2cn1]

N-(3-Bromo-1-(tetrahydro-2H-pyran-2-yl)-1H-pyrazolo[4,3-c]pyridin-6-yl)acetamide (Preparation 365, 95 g, 280.1 mmol) was added to TFA (1.0 L) at 10° C. in portions. After stirring for 12 h at 25° C., the reaction was concentrated in vacuo. The residue was poured into pre-cooled saturated aq.Na₂CO₃ (1 L), filtered and dried to afford N-(3-bromo-1H-pyrazolo[4,3-c]pyridin-6-yl)acetamide (65 g, 91.0%) as yellow solid. ¹HNMR (400 MHz, DMSO-d₆) δ ppm: 10.67 (s, 1H), 8.68 (s, 1H), 8.20 (s, 1H), 2.12 (s, 3H)

### Preparation 367: N-(3-bromo-1-(4-(1,1-difluoroethyl)pyrimidin-2-yl)-1H-pyrazolo[4,3-c]pyridin-6-yl)acetamide

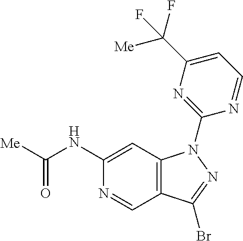 [SMILES: CC(=O)Nc1cc2c(cn1)c(Br)nn2-c1nccc(C(C)(F)F)n1]

To a solution of N-(3-bromo-1H-pyrazolo[4,3-c]pyridin-6-yl)acetamide (Preparation 366, 1.0 g, 3.92 mmol) in DMSO (10 mL) was added 2-chloro-4-(1,1-difluoroethyl)pyrimidine (Preparation 178, 700 mg, 3.92 mmol) and Cs₂CO₃ (2.6 g, 7.84 mmol) and the mixture stirred at 70° C. for 2 h. The reaction was quenched with water (30 mL) and extracted with EtOAc (2×30 mL). The combined organics were washed with brine (30 mL), dried (Na₂SO₄) and evaporated to dryness in vacuo. The residue was purified by column chromatography (SiO₂, 20-66% EtOAc/PE) to give N-(3-bromo-1-(4-(1,1-difluoroethyl)pyrimidin-2-yl)-1H-pyrazolo[4,3-c]pyridin-6-yl)acetamide as a yellow solid (850 mg, 55%). LCMS m/z=398.8 [M+H]⁺.

### Preparation 368: N-(3-bromo-1-(2-(1,1-difluoroethyl)pyrimidin-4-yl)-1H-pyrazolo[4,3-c]pyridin-6-yl)acetamide

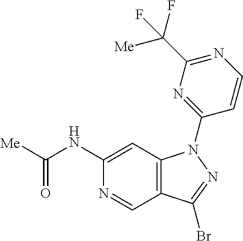 [SMILES: CC(=O)Nc1cc2c(cn1)c(Br)nn2-c1ccnc(C(C)(F)F)n1]

The title compound was prepared as a pale yellow solid (65 mg, 21%) from N-(3-bromo-1H-pyrazolo[4,3-c]pyridin-6-yl)acetamide (Preparation 366) and 4-chloro-2-(1,1-difluoroethyl)pyrimidine (Preparation 184) using an analogous method to that described for Preparation 367. LCMS m/z=398.8 [M+H]⁺.

### Preparation 369: 3-(6-bromopyrazin-2-yl)oxetan-3-ol

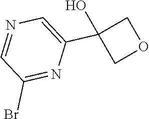 [SMILES: OC1(c2cncc(Br)n2)COC1]

n-BiLi (2.5 M, 1.68 mL) was added slowly to a solution of 2,6-dibromopyrazine (1 g, 4.20 mmol) in DCM (10 mL) at −70° C. and stirred for 30 minutes under N₂. To this was added oxetan-3-one (303 mg, 4.20 mmol) was added slowly and the reaction mixture stirred at −70° C. for 2 h under N₂. The mixture was quenched with aqueous NH₄Cl (20 mL) and extracted with EtOAc (3×20 mL). The combined organics were washed with brine (2×10 mL), dried (Na₂SO₄) and evaporated to dryness in vacuo. The residue was purified by chromatography (PE/EtOAc=3/1) on silica gel to give 3-(6-bromopyrazin-2-yl)oxetan-3-ol as a yellow oil (620 mg). ¹H NMR (400 MHz, CDCl₃) δ: 9.14 (s, 1H), 8.72 (s, 1H), 4.58-4.50 (m, 4H).

### Preparation 370: 2-bromo-6-(3-fluorooxetan-3-yl)pyrazine

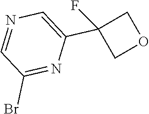 [SMILES: FC1(c2cncc(Br)n2)COC1]

To a solution of 3-(6-bromopyrazin-2-yl)oxetan-3-ol (Preparation 369, 600 mg, 2.60 mmol) in DCM (10 mL) was added DAST (837 mg, 5.19 mmol) at 0° C. and then the resulting mixture was stirred at 20° C. for 2 h. The mixture was quenched with Na₂CO₃ (10 mL), extracted with DCM (2×10 mL). The combined organics were washed with brine (2×10 mL), dried (Na₂SO₄) and concentrated under reduced pressure and the residue purified by chromatography on silica gel (PE/EtOAc=5/1) to give 2-bromo-6-(3-fluorooxetan-3-yl)pyrazine as a yellow oil (250 mg, 41%). ¹H NMR (400 MHz, CDCl₃) δ: 8.73-8.72 (m, 2H), 5.16-5.14 (m, 1H), 5.10-5.07 (m, 2H), 5.01-5.00 (m, 1H).

### Preparation 371: 3-(4,6-dichloropyrimidin-2-yl)oxetan-3-ol

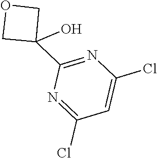 [SMILES: OC1(c2nc(Cl)cc(Cl)n2)COC1]

3-(4,6-Dichloropyrimidin-2-yl)oxetan-3-ol was obtained, 700 mg, 39.5% yield as a yellow solid, from 4,6-dichloro-2-iodopyrimidine and oxetan-3-one following the procedure described in Preparation 191. 1H NMR: (500 MHz, DMSO-d₆) δ ppm: 8.03 (s, 1H), 6.65 (s, 1H), 4.92 (d, J=7.0 Hz, 2H), 4.64 (d, J=7.0 Hz, 2H).

### Preparation 372: 3-(4-chloro-6-methoxypyrimidin-2-yl)oxetan-3-ol

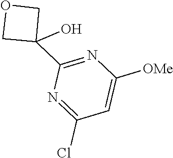 [SMILES: COc1cc(Cl)nc(C2(O)COC2)n1]

3-(4-Chloro-6-methoxypyrimidin-2-yl)oxetan-3-ol was obtained as a colorless oil, 600 mg, 87.5% yield, from 3-(4,6-dichloropyrimidin-2-yl)oxetan-3-ol (Preparation 371), following the procedure described in Preparation 205. 1H NMR (500 MHz, DMSO-d₆) δ ppm: 7.13 (s, 1H), 6.40 (s, 1H), 4.94 (d, J=7.0 Hz, 2H), 4.65-4.63 (m, 2H), 4.00 (s, 3H).

### Preparation 373: 0-(3-(4-chloro-6-methoxypyrimidin-2-yl)oxetan-3-yl) S-methyl carbonodithioate

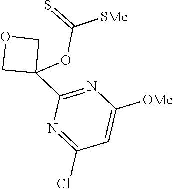 [SMILES: COc1cc(Cl)nc(C2(OC(=S)SC)COC2)n1]

O-(3-(4-Chloro-6-methoxypyrimidin-2-yl)oxetan-3-yl) S-methyl carbonodithioate was obtained as a yellow oil, 340 mg, 41.2% yield, from 3-(4-chloro-6-methoxypyrimidin-2-yl)oxetan-3-ol (Preparation 372), following the procedure described in Preparation 198. 1H NMR: (500 MHz, CDCl₃) δ ppm: 6.65 (s, 1H), 5.19 (d, J=8.0 Hz, 2H), 5.09 (d, J=8.0 Hz, 2H), 3.99 (s, 3H), 2.59 (s, 3H).

### Preparation 374: 4-chloro-6-methoxy-2-(oxetan-3-yl)pyrimidine

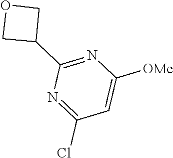 [SMILES: COc1cc(Cl)nc(C2COC2)n1]

4-Chloro-6-methoxy-2-(oxetan-3-yl)pyrimidine was obtained as a yellow oil, 70 mg, 35.7% yield, from O-(3-(4-chloro-6-methoxypyrimidin-2-yl)oxetan-3-yl) S-methyl carbonodithioate (Preparation 373), following the procedure described in Preparation 200. 1H NMR: (500 MHz, CDCl₃) δ ppm: 6.64 (s, 1H), 5.01 (m, 4H), 4.39 (m, 1H), 4.03 (s, 3H).

### Preparation 375: 2-(4,4,5,5-tetramethyl-1,3,2-dioxaborolan-2-yl)cyclopropane-1-carbonitrile

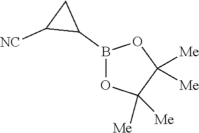 [SMILES: CC1(C)OB(C2CC2C#N)OC1(C)C]

To a solution of B₂Pin₂ (6.0 g, 23.63 mmol) and cyclopropanecarbonitrile (2.0 g, 30.72 mmol) in THF (10 mL) was added 2,9-dimethyl-1,10-phenanthroline (197 mg, 0.945 mmol), [Ir(COD)OMe]₂ (627 mg, 0.945 mmol) at 25° C. under N₂ and the mixture was heated to 90° C. with stirred for 18 h. The mixture was concentrated under reduced pressure to give a residue which was purified on silica gel column chromatography (PE/EtOAc=20/1 to 10/1) to give 2-(4,4,5,5-tetramethyl-1,3,2-dioxaborolan-2-yl)cyclopropane-1-carbonitrile as a yellow oil (800 mg, 17.5%). ¹H NMR (500 MHz, CDCl₃) δ: 1.23-1.21 (m, 1H), 1.07-1.10 (m, 12H), 0.88-0.86 (m, 2H), 0.85 (s, 1H).

### Preparation 376: 1-(2-bromo-5-methylthiazol-4-yl)ethan-1-one

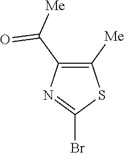

To a solution of CuBr₂ (1.7 g, 7.68 mmol) in MeCN (10 mL) was added tert-butyl nitrite (990 mg, 9.60 mmol) slowly at 0° C. and stirred at 0° C. for 10 min. The reaction was allowed to warm up to rt and 1-(2-amino-5-methylthiazol-4-yl)ethan-1-one (1 g, 6.40 mmol) was added and the resulting mixture stirred at 30° C. for 2 h. The mixture was concentrated and the residue dissolved in DCM (10 mL). The solids were removed by filtration and the organic layer extracted with Na₂CO₃ (10 mL×2), washed with brine (10 mL), dried (Na₂SO₄) and concentrated under reduced pressure to give 1-(2-bromo-5-methylthiazol-4-yl)ethan-1-one as a brown solid (1 g, 71%). ¹H NMR (500 MHz, CDCl₃) δ: 2.71 (s, 3H), 2.61 (s, 3H).

### Preparation 377: 2-bromo-4-(1,1-difluoroethyl)-5-methylthiazole

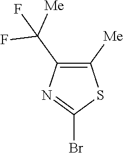 [SMILES: Cc1sc(Br)nc1C(C)(F)F]

2-Bromo-4-(1,1-difluoroethyl)-5-methylthiazole was prepared as a yellow oil (120 mg, crude) from 1-(2-bromo-5-methylthiazol-4-yl)ethan-1-one (Preparation 376) using an analogous method to that described for Preparation 182. ¹H NMR (500 MHz, CDCl₃) δ: 2.52-2.54 (m, 3H), 2.03 (t, 3H).

### Preparation 378: 2-(6-chloro-1H-pyrrolo[3,2-c]pyridin-1-yl)-5-methyl-4-(tetrahydrofuran-3-yl)thiazole

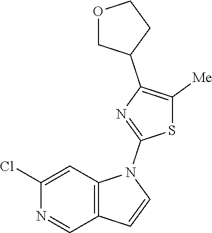 [SMILES: Cc1sc(-n2ccc3cnc(Cl)cc32)nc1C1CCOC1]

To the solution of 6-chloro-1H-pyrrolo[3,2-c]pyridine and 2-bromo-5-methyl-4-(tetrahydrofuran-3-yl)thiazole (162.6 mg, 0.655 mmol) in dioxane (10 mL) was added N1,N2-dimethylethane-1,2-diamine (11.6 mg, 0.131 mmol), CuI (49.9 mg, 0.262 mmol) and K₂CO₃ (181.2 mg, 1.31 mmol) and the mixture was heated at 90° C. for 2 h under N₂. The residue was purified by column chromatography on silica gel (PE/EtOAc=5/1) to give 2-(6-chloro-1H-pyrrolo[3,2-c]pyridin-1-yl)-5-methyl-4-(tetrahydrofuran-3-yl)thiazole as a yellow solid (145 mg, 69%). ¹H NMR (400 MHz, CDCl₃) δ: 8.69 (s, 1H), 8.17 (s, 1H), 7.56 (d, 1H), 6.74 (d, 1H), 4.18-4.12 (m, 2H), 4.03-4.00 (m, 1H), 3.87-3.83 (m, 1H), 3.55-3.51 (m, 1H), 2.44 (s, 3H) 2.35-2.27 (m, 2H).

### Preparation 379: 1-(5-methyl-4-(tetrahydrofuran-3-yl)thiazol-2-yl)-1H-pyrrolo[3,2-c]pyridin-6-amine

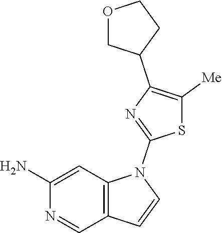 [SMILES: Cc1sc(-n2ccc3cnc(N)cc32)nc1C1CCOC1]

A mixture of 2-(6-chloro-1H-pyrrolo[3,2-c]pyridin-1-yl)-5-methyl-4-(tetrahydrofuran-3-yl)thiazole (Preparation 378, 133 mg, 0.416 mmol), Pd(OAc)₂ (9.3 mg, 0.042 mmol), NH₂Boc (97.4 mg, 0.832 mmol), BINAP (51.8 mg, 0.083 mmol) and Cs₂CO₃ (271 mg, 0.832 mmol) in dioxane (10 mL) was stirred at 110° C. for 12 h under N₂. The mixture was purified by prep-HPLC-G (55-85% MeCN) to give 1-(5-methyl-4-(tetrahydrofuran-3-yl)thiazol-2-yl)-1H-pyrrolo[3,2-c]pyridin-6-amine as a yellow solid (15 mg, 12%). LCMS m/z=301.1 [M+H]⁺.

### Preparation 380: 2-(6-chloro-1H-pyrrolo[3,2-c]pyridin-1-yl)-4-(tetrahydrofuran-3-yl)thiazole

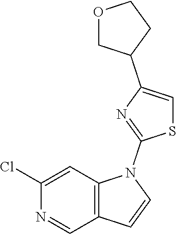 [SMILES: Clc1cc2c(ccn2-c2nc(C3CCOC3)cs2)cn1]

2-(6-Chloro-1H-pyrrolo[3,2-c]pyridin-1-yl)-4-(tetrahydrofuran-3-yl)thiazole was obtained as a colorless oil, 66 mg, 50.5% yield, from 6-chloro-1H-pyrrolo[3,2-c]pyridine and 2-bromo-4-(tetrahydrofuran-3-yl)thiazole, following the procedure described in Preparation 378. 1H NMR: (400 MHz, CDCl₃) δ: ppm 8.70 (d, J=2.8 Hz, 1H), 8.25 (s, 1H), 7.62 (d, J=4.0 Hz, 1H), 6.80 (s, 1H), 6.81 (d, J=2.8 Hz, 1H), 4.22-4.15 (m, 1H), 4.11-4.04 (m, 1H), 4.03-3.98 (m, 1H), 3.96-3.91 (m, 1H), 3.66-3.63 (m, 1H), 2.46-2.33 (m, 1H), 2.28-2.15 (m, 1H).

### Preparation 381: 1-(3-bromophenyl)-2,2,2-trifluoroethan-1-amine hydrochloride

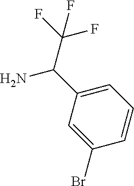 [SMILES: NC(c1cccc(Br)c1)C(F)(F)F]

To a solution of 1-(3-bromophenyl)-2,2,2-trifluoroethan-1-one (2 g, 7.90 mmol) in toluene (30 mL) was added LiHMDS (1 M, 8.7 mL) over 10 min and the reaction mixture was stirred at 30° C. for 30 min. BH₃·Me₂S (10 M, 1.58 mL) was added to the mixture and the mixture stirred for another 30 min. After cooling to 0° C., NaOH (2 M, 7.9 mL) was carefully added dropwise over 5 min and the resulting mixture stirred at 30° C. for 90 min. The layers were separated and the organic layer washed with NaOH (2 M, 5 mL) and water, dried (Na₂SO₄) and filtered. The solution was treated with HCl/dioxane (4 M, 5 mL) and the mixture was concentrated under reduced pressure to afford 1-(3-bromophenyl)-2,2,2-trifluoroethan-1-amine hydrochloride as a white solid (1.9 g, 95%). ¹H NMR (400 MHz, DMSO-d₆) δ: 9.73 (br s, 2H), 7.93 (s, 1H), 7.74 (d, 1H), 7.68 (d, 1H), 7.49 (t, 1H), 5.62-5.56 (m, 1H).

### Preparation 382: tert-butyl (1-(3-bromophenyl)-2,2,2-trifluoroethyl)carbamate

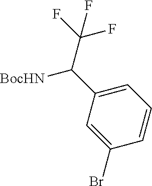 [SMILES: CC(C)(C)OC(=O)NC(c1cccc(Br)c1)C(F)(F)F]

To a solution of 1-(3-bromophenyl)-2,2,2-trifluoroethan-1-amine hydrochloride (Preparation 381, 1.0 g, 3.94 mmol) in DCM (10 mL) was added TEA (797 mg, 7.87 mmol) and (Boc)₂O (859 mg, 3.94 mmol) and the resulting mixture was stirred at 30° C. for 4 h. The reaction was diluted with H₂O (10 mL) and extracted with DCM (3×15 mL). The combined organics were washed with brine (2×10 mL), dried (Na₂SO₄) and concentrated under reduced pressure. The residue was purified by chromatography on silica gel (PE/EtOAc=3/1) to give tert-butyl (1-(3-bromophenyl)-2,2,2-trifluoroethyl)carbamate as a white solid (1 g, 72%). ¹H NMR (500 MHz, CDCl₃) δ: 7.52-7.47 (m, 2H), 7.29-7.28 (m, 1H), 7.23-7.21 (m, 1H), 5.54-5.45 (m, 1H), 1.52 (s, 9H).

### Preparation 383: tert-butyl (1-(3-(6-acetamido-1H-pyrazolo[4,3-c]pyridin-1-yl)phenyl)-2,2,2-trifluoroethyl)carbamate

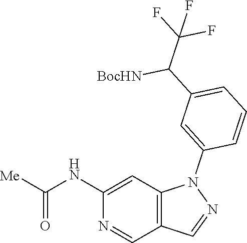 [SMILES: CC(=O)Nc1cc2c(cn1)cnn2-c1cccc(C(NC(=O)OC(C)(C)C)C(F)(F)F)c1]

A mixture of tert-butyl (1-(3-bromophenyl)-2,2,2-trifluoroethyl)carbamate (Preparation 382, 300 mg, 0.847 mmol), N-(1H-pyrazolo[4,3-c]pyridin-6-yl)acetamide (Preparation 2, 149 mg, 0.847 mmol), Pd₂(dba)₃ (77.6 mg, 0.085 mmol), Cs₂CO₃ (552 mg, 1.69 mmol) and tBuXPhos (36 mg, 0.085 mmol) in toluene (5 mL) was stirred at 90° C. for 2 h under N₂. The mixture was concentrated and purified by chromatography on silica gel (PE/EtOAc=1/1) to give a yellow solid which was further purified by HPLC-C (38-68% MeCN) to give tert-butyl (1-(3-(6-acetamido-1H-pyrazolo[4,3-c]pyridin-1-yl)phenyl)-2,2,2-trifluoroethyl)carbamate as a white solid (15 mg, 4%). ¹H NMR (500 MHz, CDCl₃) δ: 8.80 (s, 1H), 8.58 (s, 1H), 8.43 (s, 1H), 8.24 (s, 1H), 7.79-7.75 (m, 2H), 7.58 (t, 1H), 7.40 (d, 1H), 5.57-5.47 (m, 2H), 2.24 (s, 3H), 1.46 (s, 9H).

### Preparation 384: ((3-(3-bromophenyl)oxetan-3-yl)methoxy)(tert-butyl)dimethylsilane

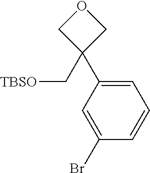 [SMILES: CC(C)(C)[Si](C)(C)OCC1(c2cccc(Br)c2)COC1]

To a solution of (3-(3-bromophenyl)oxetan-3-yl)methanol (100 mg, 0.411 mmol) and imidazole (56 mg, 0.823 mmol) in DCM (3 mL) was added TBSCl (74.4 mg, 0.494 mmol) at 0° C. The mixture was stirred at 25° C. for 2 h, diluted with water (10 mL) and extracted with EtOAc (10 mL×3). The combined organics were washed with brine (10 mL), dried (Na₂SO₄) and evaporated to dryness in vacuo. The residue was purified by prep-TLC (25% EtOAc/PE) to give ((3-(3-bromophenyl)oxetan-3-yl)methoxy)(tert-butyl)dimethylsilane as a colourless oil (101.3 mg, 69%). ¹H NMR (500 MHz, CDCl₃) δ: 7.39-7.37 (m, 1H), 7.22-7.18 (m, 2H), 6.97 (d, 1H), 4.88 (d, 2H), 4.74 (d, 2H), 3.93 (s, 2H), 0.84 (s, 9H), −0.09 (s, 6H).

### Preparation 385: N-(1-(3-(3-(((tert-butyldimethylsilyl)oxy)methyl)oxetan-3-yl)phenyl)-1H-pyrazolo[4,3-c]pyridin-6-yl)acetamide

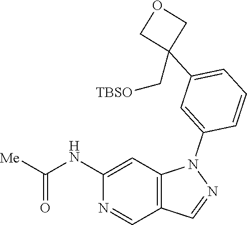 [SMILES: CC(=O)Nc1cc2c(cn1)cnn2-c1cccc(C2(CO[Si](C)(C)C(C)(C)C)COC2)c1]

To a solution of ((3-(3-bromophenyl)oxetan-3-yl)methoxy)(tert-butyl)dimethylsilane (Preparation 384, 60 mg, 0.168 mmol) and N-(1H-pyrazolo[4,3-c]pyridin-6-yl)acetamide (Preparation 2, 35.5 mg, 0.201 mmol) in toluene (3 mL) was added BrettPhos Pd G₃ (15.2 mg, 0.017 mmol) and Cs₂CO₃ (109.4 mg, 0.336 mmol) and the resulting mixture stirred at 110° C. for 12 h under N₂. The mixture was poured into H₂O (10 mL) and extracted with EtOAc (10 mL×3). The combined organics were dried (Na₂SO₄) and evaporated to dryness. The residue was purified by chromatography (PE/EtOAc=15/1 to 1/1) on silica gel to give N-(1-(3-(3-(((tert-butyldimethylsilyl)oxy)methyl)oxetan-3-yl)phenyl)-1H-pyrazolo[4,3-c]pyridin-6-yl)acetamide (68.1 mg, 90%) as colourless oil. ¹H NMR (400 MHz, CDCl₃) δ: 8.81 (s, 1H), 8.59 (s, 1H), 8.24 (s, 1H), 8.06 (s, 1H), 7.62 (d, 1H), 7.22 (d, 1H), 7.45 (s, 1H), 7.10 (d, 1H), 5.00 (d, 2H), 4.85 (d, 2H), 4.00 (s, 2H), 2.24 (s, 3H), 0.82 (s, 9H), −0.06 (s, 6H).

### Preparation 386: N-(3-bromo-1-(4-(1,1-difluoroethyl)pyrimidin-2-yl)-1H-pyrrolo[3,2-c]pyridin-6-yl)acetamide

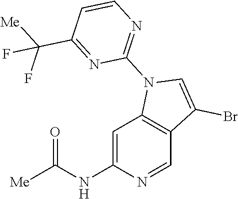 [SMILES: CC(=O)Nc1cc2c(cn1)c(Br)cn2-c1nccc(C(C)(F)F)n1]

To a solution of N-(3-bromo-1H-pyrrolo[3,2-c]pyridin-6-yl)acetamide (Preparation 24, 1.0 g, 3.94 mmol) in DMF (10 mL) was added 2-chloro-4-(1,1-difluoroethyl)pyrimidine (Preparation 178, 843 mg, 4.72 mmol), Cs₂CO₃ (2.6 g, 7.87 mmol) and the mixture stirred at 80° C. for 2 h. The mixture was diluted with H₂O (20 mL) and extracted with EtOAc (20 mL×3). The combined organics were washed with brine (20 mL×3), dried (Na₂SO₄) and evaporated to dryness in vacuo and the residue purified by column chromatography (SiO₂, PE/EtOAc=1/0 to 0/1) to give N-(3-bromo-1-(4-(1,1-difluoroethyl)pyrimidin-2-yl)-1H-pyrrolo[3,2-c]pyridin-6-yl)acetamide as a yellow solid (1.1 g, 72.5%). ¹H NMR (500 MHz, CDCl₃) δ: 9.49 (s, 1H), 8.90 (d, 1H), 8.52 (s, 1H), 8.30 (s, 1H), 8.12 (s, 1H), 7.47 (d, 1H), 2.27-2.19 (m, 6H).

### Preparation 387: N-(3-bromo-1-(2-(1,1-difluoroethyl)pyrimidin-4-yl)-1H-pyrrolo[3,2-c]pyridin-6-yl)acetamide

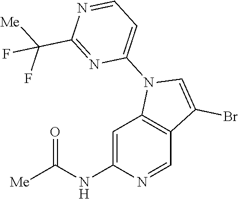 [SMILES: CC(=O)Nc1cc2c(cn1)c(Br)cn2-c1ccnc(C(C)(F)F)n1]

N-(3-Bromo-1-(2-(1,1-difluoroethyl)pyrimidin-4-yl)-1H-pyrrolo[3,2-c]pyridin-6-yl)acetamide was prepared as a white solid (194 mg, 83%) from N-(3-bromo-1H-pyrrolo[3,2-c]pyridin-6-yl)acetamide (Preparation 24) and 4-chloro-2-(1,1-difluoroethyl)pyrimidine (Preparation 184) using an analogous method to that described for Preparation 385. ¹H NMR (500 MHz, CDCl₃) δ: 9.25 (s, 1H), 8.92 (d, 1H), 8.55 (s, 1H), 8.17 (s, 1H), 7.90 (d, 1H), 7.51 (d, 1H), 2.27-2.18 (m, 6H).

Preparation 388: (Z)-3-aminopent-2-enamide

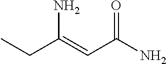

NH₃ (g) was introduced to a solution of ethyl 3-oxopentanoate (500 g, 3.47 mol) in xylene (2.0 L). The mixture was refluxed at 100° C. for 12 h with concomitant azeotropic removal of H₂O generated in the reaction. The mixture was cooled to rt and the mixture evaporated under reduced pressure to give (Z)-3-aminopent-2-enamide as a yellow solid, that was used without further purification.

### Preparation 389: 2-(1,1-difluoroethyl)-6-ethylpyrimidin-4-ol

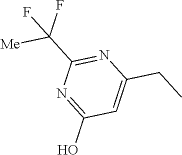

(Z)-3-Aminopent-2-enamide (Preparation 388, 395.9 g, 3.47 mol) was dissolved in EtOH (2.5 L), t-BuONa (500 g, 5.2 mol) was added in batches, followed by ethyl 2,2-difluoropropionate (503 g, 3.64 mol) and the resulting black solution was heated to 85° C. and stirred for 24 h. The mixture was cooled to 25° C., the pH adjusted to 2-3 with 2 N aq. HCl and the solvent was evaporated under reduced pressure. EtOAc (1.0 L) was added and the layers separated. The organic layer was dried over anhydrous MgSO₄, filtered and concentrated to give 2-(1,1-difluoroethyl)-6-ethylpyrimidin-4-ol as a brown liquid (385.2 g, 59.0% yield). 1HNMR (400 MHz, DMSO-d₆) δ (ppm): 6.46 (s, 1H), 2.60-2.50 (m, 2H), 2.00-1.90 (m, 3H), 1.20-1.18 (m, 3H).

### Preparation 390: 4-chloro-2-(1,1-difluoroethyl)-6-ethylpyrimidine

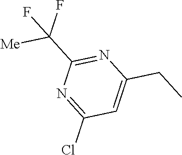

2-(1,1-Difluoroethyl)-6-ethylpyrimidin-4-ol (Preparation 389, 385 g, 2.05 mol) was dissolved in MeCN (1 L), then POCl3 (313.7 g, 2.05 mol) was added and the reaction heated to 80° C. and stirred for 4 h. The mixture was cooled to 30° C. and concentrated under vacuum to remove most of solvent. The residue was dissolved in EtOAc (1.0 L), poured into ice water, maintaining the internal temperature between 20° C. to 30° C. The mixture was separated, the organic phase was washed with brine (2×), dried over anhydrous MgSO4, filtered and concentrated. The crude product was distilled (external temp.: 50° C.; internal temp.: 25-30° C.) to remove impurities. The residue was purified by silica gel chromatography eluting with n-Heptane/EtOAc (40/1) to give 4-chloro-2-(1,1-difluoroethyl)-6-ethylpyrimidine (110.4 g, 26.1% yield) as yellow liquid. LCMS m/z=207.0 [M+H]⁺

### Preparation 391: N-(3-bromo-1-(2-(1,1-difluoroethyl)-6-methylpyrimidin-4-yl)-1H-pyrazolo[4,3-c]pyridin-6-yl)acetamide

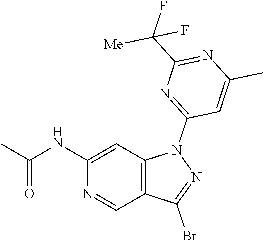 [SMILES: CC(=O)Nc1cc2c(cn1)c(Br)nn2-c1cc(C)nc(C(C)(F)F)n1]

To a solution of N-(3-bromo-1H-pyrazolo[4,3-c]pyridin-6-yl)acetamide (Preparation 366, 1.0 g, 3.92 mmol) and Cs₂CO₃ (3.8 g, 11.76 mmol) in DMF (10.0 mL) was added 4-chloro-2-(1,1-difluoroethyl)-6-methylpyrimidine (Preparation 218, 830.6 mg, 4.31 mmol) and the reaction stirred at 100° C. for 1 h. The mixture was diluted with water (60 mL), extracted with EtOAc (80 mL×3) and the combined organic phase was washed with brine (120 mL), dried over anhydrous Na₂SO₄, filtered and concentrated. The crude was purified by silica gel column chromatography (PE/EtOAc=0/1 to 1/1) to give N-(3-bromo-1-(2-(1,1-difluoroethyl)-6-methylpyrimidin-4-yl)-1H-pyrazolo[4,3-c]pyridin-6-yl)acetamide (1.2 g, 71.3% yield) as a yellow solid. ¹H NMR: (400 MHz, CDCl₃) δ ppm 9.54 (s, 1H), 8.67 (s, 1H), 8.22 (br s, 1H), 7.84 (s, 1H), 2.70 (s, 3H), 2.24-2.33 (m, 6H).

### Preparation 392: N-(1-(2-(1,1-difluoroethyl)-6-methylpyrimidin-4-yl)-3-iodo-1H-pyrazolo[4,3-c]pyridin-6-yl)acetamide

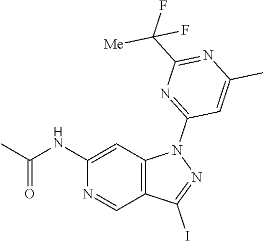 [SMILES: CC(=O)Nc1cc2c(cn1)c(I)nn2-c1cc(C)nc(C(C)(F)F)n1]

To a solution of 4-chloro-2-(1,1-difluoroethyl)-6-methylpyrimidine (Preparation 218, 637.6 mg, 3.31 mmol) and N-(3-iodo-1H-pyrazolo[4,3-c]pyridin-6-yl)acetamide (Preparation 3, 1 g, 3.31 mmol) in DMF (10 mL) was added Cs₂CO₃ (2.16 g, 6.62 mmol) and the reaction mixture stirred at 70° C. for 2 h. The mixture was cooled to rt and diluted with water. The resulting solid was filtered off and dried under vacuum to give N-(1-(2-(1,1-difluoroethyl)-6-methylpyrimidin-4-yl)-3-iodo-1H-pyrazolo[4,3-c]pyridin-6-yl)acetamide (873 mg, 57.6% yield) as a brown solid. LCMS m/z=458.9 [M+H]⁺.

### Preparation 393: N-(3-bromo-1-(2-(1,1-difluoroethyl)-6-ethylpyrimidin-4-yl)-1H-pyrazolo[4,3-c]pyridin-6-yl)acetamide

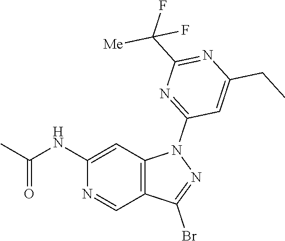 [SMILES: CCc1cc(-n2nc(Br)c3cnc(NC(C)=O)cc32)nc(C(C)(F)F)n1]

To a solution of N-(3-bromo-1H-pyrazolo[4,3-c]pyridin-6-yl)acetamide (Preparation 366, 1.5 g, 5.88 mmol) in DMF (15 mL) was added Cs₂CO₃ (5.75 g, 17.6 mmol) and 4-chloro-2-(1,1-difluoroethyl)-6-ethylpyrimidine (Preparation 390, 1.22 g, 5.88 mmol) and the reaction stirred at 25° C. for 2 h. The reaction mixture was diluted with H₂O (20 mL), filtered and the filtrate evaporated under reduced pressure to give N-(3-bromo-1-(2-(1,1-difluoroethyl)-6-ethylpyrimidin-4-yl)-1H-pyrazolo[4,3-c]pyridin-6-yl)acetamide (1.44 g, 51.8% yield), which was used for the next step without purification. ¹H NMR: (400 MHz, DMSO-d₆) δ ppm 10.94 (s, 1H), 9.42 (s, 1H), 8.84 (s, 1H), 7.89 (s, 1H), 2.93 (q, J=7.6 Hz, 2H), 2.17-2.29 (m, 6H), 2.17 (s, 3H), 1.30 (t, J=7.6 Hz, 3H).

### Preparation 394: N-(1-benzyl-3-methylpyrrolidin-3-yl)acetamide

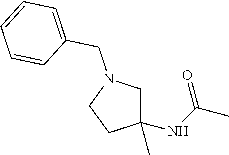 [SMILES: CC(=O)NC1(C)CCN(Cc2ccccc2)C1]

Sulfuric acid (16 M, 16.0 mL) was added dropwise to 1-benzyl-3-methylpyrrolidin-3-ol (4.0 g, 20.91 mmol) in MeCN (12.0 mL) at 0° C. The solution was allowed to warm up to rt then stirred for 16 h. The mixture was poured into ice, the pH adjusted to 7 using sat. aq. NaOH and the mixture extracted with DCM (50 ml×3). The organic layer was washed with brine (30 mL), dried over Na₂SO₄ and concentrated in vacuo to give N-(1-benzyl-3-methylpyrrolidin-3-yl)acetamide (4.0 g, 82.3% yield) as a yellow solid. LCMS m/z=233.2 [M+H]⁺.

### Preparation 395: 1-benzyl-N-ethyl-3-methylpyrrolidin-3-amine

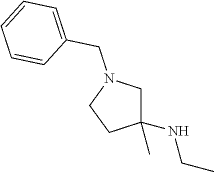 [SMILES: CCNC1(C)CCN(Cc2ccccc2)C1]

To a solution of N-(1-benzyl-3-methylpyrrolidin-3-yl)acetamide (Preparation 394, 2.0 g, 8.61 mmol) in THF (40 mL) was added LiAlH₄ (1.3 g, 34.4 mmol) at 0° C. and the reaction was stirred at 60° C. for 7 h. The solution was cooled to 0° C., quenched with water (1.5 mL) then 15% NaOH aq (1.5 mL) and water (4.5 mL) were added. The mixture was dried over Na₂SO₄ (1.0 g) the mixture stirred for 0.5 h, filtered, washing through with THF. The filtrate was evaporated under reduced pressure to give 1-benzyl-N-ethyl-3-methylpyrrolidin-3-amine (1.9 g, crude) as yellow oil. LCMS m/z=219.2 [M+H]⁺.

### Preparation 396: 1-benzyl-N-ethyl-N,3-dimethylpyrrolidin-3-amine

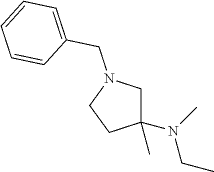 [SMILES: CCN(C)C1(C)CCN(Cc2ccccc2)C1]

To a solution of 1-benzyl-N-ethyl-3-methylpyrrolidin-3-amine (Preparation 395, 1.2 g, 5.50 mmol) in MeOH (15.0 mL) was added NaBH₃CN (1.7 g, 27.5 mmol) and (CH₂O)ₙ(22.48 mL, 16.5 mmol) and the reaction mixture stirred at 25° C. for 1 h. The mixture was concentrated under reduced pressure and the residue was purified by Prep-HPLC-A (Gradient 24 to 54% MeCN) to give 1-benzyl-N-ethyl-N,3-dimethylpyrrolidin-3-amine (570 mg, 44.6% yield) as a yellow gum. LCMS m/z=233.2 [M+H]⁺.

### Preparation 397: 1-benzyl-N,N-diethyl-3-methylpyrrolidin-3-amine

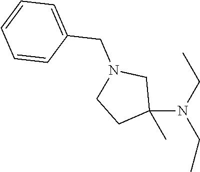 [SMILES: CCN(CC)C1(C)CCN(Cc2ccccc2)C1]

The title compound was obtained as a colorless oil, 380 mg, 22.6%, from 1-benzyl-3-methylpyrrolidin-3-amine and acetaldehyde, following a similar procedure to that described in Preparation 396. LCMS m/z=247.2 [M+H]⁺.

### Preparation 398: N-ethyl-N,3-dimethylpyrrolidin-3-amine acetate

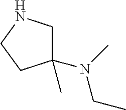

To a solution of 1-benzyl-N-ethyl-N,3-dimethylpyrrolidin-3-amine (Preparation 396, 570 mg, 2.61 mmol) in MeOH (10 mL) was added Pd/C (1.4 g, 1.31 mmol, 10% purity) and AcOH (1.1 g, 17.5 mmol) and the reaction mixture was stirred at 60° C. for 16 h under H₂ (50 Psi). Pd/C was filtered off and the mixture was concentrated to give N-ethyl-N,3-dimethylpyrrolidin-3-amine acetate (1.12 g, crude) as yellow oil. ¹H NMR: (500 MHz, CDCl₃) δ ppm: 3.33-3.42 (m, 3H), 3.10-3.15 (m, 1H), 2.56-2.61 (m, 2H), 2.38 (s, 3H), 2.22-2.29 (m, 1H), 1.86-1.88 (m, 1H), 1.26 (s, 3H), 1.17 (t, J=7.0 Hz, 3H).

### Preparation 399: N,N-diethyl-3-methylpyrrolidin-3-amine acetate

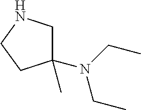

N,N-Diethyl-3-methylpyrrolidin-3-amine acetate was obtained as a yellow oil, 265.1 mg, crude, from 1-benzyl-N,N-diethyl-3-methylpyrrolidin-3-amine (Preparation 397) following the procedure described in Preparation 398. ¹H NMR (400 MHz, MeOD-d₄) δ ppm: 3.40-3.48 (m, 1H), 3.19-3.21 (m, 1H), 2.98-3.05 (m, 2H), 2.67-2.78 (m, 4H), 2.22-2.26 (m, 1H), 1.92-1.93 (m, 1H), 1.27 (s, 3H), 1.15 (t, J=7.2 Hz, 6H).

### Preparation 400: tert-butyl ((3R,4S)-1-(6-acetamido-1-(2-(1,1-difluoroethyl)-6-methylpyrimidin-4-yl)-1H-pyrazolo[4,3-c]pyridin-3-yl)-4-methylpyrrolidin-3-yl)carbamate

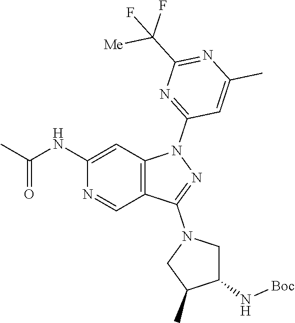 [SMILES: CC(=O)Nc1cc2c(cn1)c(N1C[C@H](NC(=O)OC(C)(C)C)[C@@H](C)C1)nn2-c1cc(C)nc(C(C)(F)F)n1]

To a solution of N-(3-bromo-1-(2-(1,1-difluoroethyl)-6-methylpyrimidin-4-yl)-1H-pyrazolo[4,3-c]pyridin-6-yl)acetamide (Preparation 391, 200 mg, 0.49 mmol), CuI (9.3 mg, 0.049 mmol), K₃PO₄ (206.5 mg, 0.97 mmol) and 2-(2,6-difluoroanilino)-2-oxo-acetic acid (19.6 mg, 0.097 mmol) in DMSO (2 mL) was added tert-butyl ((3R,4S)-4-methylpyrrolidin-3-yl)carbamate (97.4 mg, 0.49 mmol) and the reaction stirred at 100° C. for 12 h under N₂. The mixture was concentrated, the residue diluted with water (50 mL) and extracted with EtOAc (50 mL×3). The combined organic phase was washed with brine (50 mL), dried over anhydrous Na₂SO₄, filtered and concentrated. The crude product was purified by silica gel column chromatography (PE/EtOAc=1/0 to 1/1) to give tert-butyl ((3R,4S)-1-(6-acetamido-1-(2-(1,1-difluoroethyl)-6-methylpyrimidin-4-yl)-1H-pyrazolo[4,3-c]pyridin-3-yl)-4-methylpyrrolidin-3-yl)carbamate (160 mg, 62.0%) as yellow solid. LCMS m/z=531.2 [M+H]⁺.

### Preparation 401 to 403

| Preparation No | Name/Structure/Data |
|---|---|
| 401 | tert-butyl ((3S,4R)-1-(6-acetamido-1-(2-(1,1-difluoroethyl)-6- |
| | methylpyrimidin-4-yl)-1H-pyrazolo[4,3-c]pyridin-3-yl)-4-methylpyrrolidin- |
| | 3-yl)carbamate |
| | 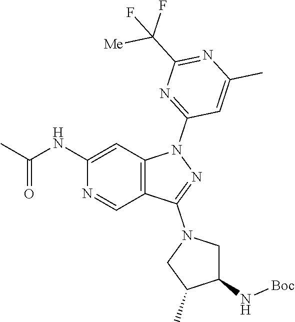 [SMILES: CC(=O)Nc1cc2c(cn1)c(N1C[C@@H](C)[C@H](NC(=O)OC(C)(C)C)C1)nn2-c1cc(C)nc(C(C)(F)F)n1] |
| | SM: tert-butyl ((3S,4R)-4-methylpyrrolidin-3-yl)carbamate |
| | Yellow solid, 210 mg, 94.2% yield |
| | LCMS m/z = 531.2 [M + H]⁺ |
| 402 | tert-butyl ((3S,4S)-1-(6-acetamido-1-(2-(1,1-difluoroethyl)-6- |
| | methylpyrimidin-4-yl)-1H-pyrazolo[4,3-c]pyridin-3-yl)-4-methylpyrrolidin- |
| | 3-yl)carbamate |
| | 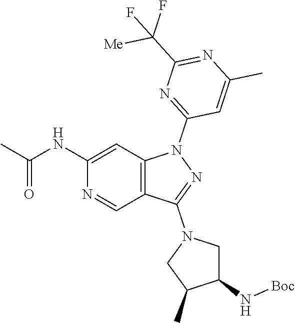 [SMILES: CC(=O)Nc1cc2c(cn1)c(N1C[C@H](C)[C@H](NC(=O)OC(C)(C)C)C1)nn2-c1cc(C)nc(C(C)(F)F)n1] |
| | SM: tert-butyl ((3S,4S)-4-methylpyrrolidin-3-yl)carbamate |
| | Yellow solid, 560 mg, 83.5%. |
| | LCMS m/z = 531.2 [M + H]⁺ |
| 403 | tert-butyl ((3R,4R)-1-(6-acetamido-1-(2-(1,1-difluoroethyl)-6- |
| | methylpyrimidin-4-yl)-1H-pyrazolo[4,3-c]pyridin-3-yl)-4-methylpyrrolidin- |
| | 3-yl)carbamate |
| | 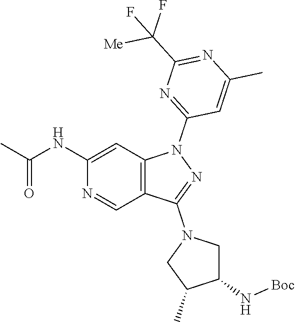 [SMILES: CC(=O)Nc1cc2c(cn1)c(N1C[C@@H](C)[C@@H](NC(=O)OC(C)(C)C)C1)nn2-c1cc(C)nc(C(C)(F)F)n1] |
| | SM: tert-butyl ((3R,4R)-4-methylpyrrolidin-3-yl)carbamate |
| | Yellow solid, 420 mg, 65.1% yield |
| | LCMS m/z = 531.2 [M + H]⁺ |

### Preparation 404: N-(3-((3R,4S)-3-amino-4-methylpyrrolidin-1-yl)-1-(2-(1,1-difluoroethyl)-6-methylpyrimidin-4-yl)-1H-pyrazolo[4,3-c]pyridin-6-yl)acetamide hydrochloride

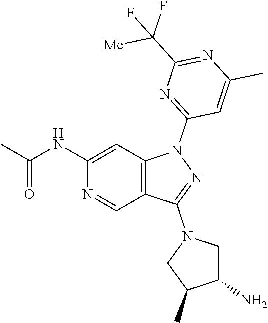 [SMILES: CC(=O)Nc1cc2c(cn1)c(N1C[C@H](C)[C@@H](N)C1)nn2-c1cc(C)nc(C(C)(F)F)n1]

To a solution of tert-butyl ((3R,4S)-1-(6-acetamido-1-(2-(1,1-difluoroethyl)-6-methylpyrimidin-4-yl)-1H-pyrazolo[4,3-c]pyridin-3-yl)-4-methylpyrrolidin-3-yl)carbamate (Preparation 400, 150 mg, 0.283 mmol) in DCM (5 mL) was added HCl/dioxane (4 M, 10 mL). The mixture was stirred at 25° C. for 2 h then evaporated under reduced pressure to give N-(3N-((3R,4-S)-3-amino-4-methylpyrrolidin-1-yl)-1-(2-(1,1-difluoroethyl)-6-methylpyrimidin-4-yl)-1H-pyrazolo[4,3-c]pyridin-6-yl)acetamide hydrochloride (130 mg, crude) as a white solid. LCMS m/z=431.2 [M+H]⁺

### Preparation 405 to 407

| Preparation No | Name/Structure/Data |
|---|---|
| 405 | N-(3-((3S,4R)-3-amino-4-methylpyrrolidin-1-yl)-1-(2-(1,1-difluoroethyl)- |
| | 6-methylpyrimidin-4-yl)-1H-pyrazolo[4,3-c]pyridin-6-yl)acetamide |
| | hydrochloride |
| | 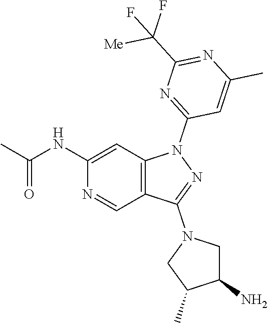 [SMILES: CC(=O)Nc1cc2c(cn1)c(N1C[C@@H](C)[C@H](N)C1)nn2-c1cc(C)nc(C(C)(F)F)n1] |
| | SM: tert-butyl ((3S,4R)-1-(6-acetamido-1-(2-(1,1-difluoroethyl)-6- |
| | methylpyrimidin-4-yl)-1H-pyrazolo[4,3-c]pyridin-3-yl)-4-methylpyrrolidin- |
| | 3-yl)carbamate (Preparation 401) |
| | Yellow solid, 90 mg crude. |
| | LCMS m/z = 431.3 [M + H]⁺ |
| 406 | N-(3-((3S,4S)-3-amino-4-methylpyrrolidin-1-yl)-1-(2-(1,1-difluoroethyl)-6- |
| | methylpyrimidin-4-yl)-1H-pyrazolo[4,3-c]pyridin-6-yl)acetamide |
| | hydrochloride |
| | 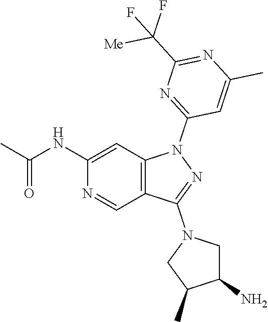 [SMILES: CC(=O)Nc1cc2c(cn1)c(N1C[C@@H](N)[C@@H](C)C1)nn2-c1cc(C)nc(C(C)(F)F)n1] |
| | SM: tert-butyl ((3S,4S)-1-(6-acetamido-1-(2-(1,1-difluoroethyl)-6- |
| | methylpyrimidin-4-yl)-1H-pyrazolo[4,3-c]pyridin-3-yl)-4-methylpyrrolidin- |
| | 3-yl)carbamate (Preparation 402) |
| | Yellow solid, 224 mg, crude |
| | LCMS m/z = 431.3 [M + H]⁺ |
| 407 | N-(3-((3R,4R)-3-amino-4-methylpyrrolidin-1-yl)-1-(2-(1,1-difluoroethyl)- |
| | 6-methylpyrimidin-4-yl)-1H-pyrazolo[4,3-c]pyridin-6-yl)acetamide |
| | hydrochloride |
| | 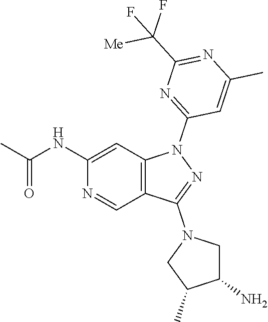 [SMILES: CC(=O)Nc1cc2c(cn1)c(N1C[C@@H](C)[C@@H](N)C1)nn2-c1cc(C)nc(C(C)(F)F)n1] |
| | SM: tert-butyl ((3R,4R)-1-(6-acetamido-1-(2-(1,1-difluoroethyl)-6- |
| | methylpyrimidin-4-yl)-1H-pyrazolo[4,3-c]pyridin-3-yl)-4-methylpyrrolidin- |
| | 3-yl)carbamate (Preparation 403) |
| | Yellow solid, 457 mg, crude |
| | LCMS m/z = 431.2 [M + H]⁺ |

### Preparation 408: 3-(6-chloro-1-(tetrahydro-2H-pyran-2-yl)-1H-pyrazolo[4,3-c]pyridin-3-yl)-6-oxa-3-azabicyclo[3.1.1]heptane

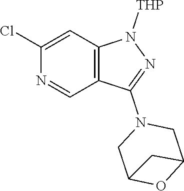

To a solution of 3-bromo-6-chloro-1-(tetrahydro-2H-pyran-2-yl)-1H-pyrazolo[4,3-c]pyridine (300 mg, 0.95 mmol) and 6-oxa-3-azabicyclo[3.1.1]heptane (154.2 mg, 1.14 mmol) in dioxane (10 mL) was added Pd₂(dba)₃ (86.8 mg, 0.095 mmol), Xantphos (109.7 mg, 0.19 mmol) and Cs₂CO₃ (926.3 mg, 2.84 mmol) and the reaction stirred at 100° C. for 4 h. The mixture was concentrated in vacuo and the crude was purified by silica gel column (EtOAc in PE from 0% to 15%) to give 3-(6-chloro-1-(tetrahydro-2H-pyran-2-yl)-1H-pyrazolo[4,3-c]pyridin-3-yl)-6-oxa-3-azabicyclo[3.1.1]heptane (220 mg, 69.4% yield) as yellow oil. LCMS m/z=335.1 [M+H]⁺.

### Preparation 409: N-(3-(6-oxa-3-azabicyclo[3.1.1]heptan-3-yl)-1-(tetrahydro-2H-pyran-2-yl)-1H-pyrazolo[4,3-c]pyridin-6-yl)acetamide

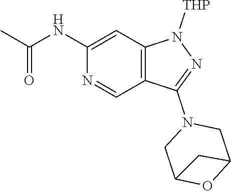

N-(3-(6-oxa-3-azabicyclo[3.1.1]heptan-3-yl)-1-(tetrahydro-2H-pyran-2-yl)-1H-pyrazolo[4,3-c]pyridin-6-yl)acetamide was obtained as a yellow solid, 200 mg, 85.2%, from 3-(6-chloro-1-(tetrahydro-2H-pyran-2-yl)-1H-pyrazolo[4,3-c]pyridin-3-yl)-6-oxa-3-azabicyclo[3.1.1]heptane (Preparation 408) and acetamide, following a similar procedure to that described in Preparation 1. LCMS m/z=358.2 [M+H]⁺.

### Preparation 410: N-(3-(6-oxa-3-azabicyclo[3.1.1]heptan-3-yl)-1H-pyrazolo[4,3-c]pyridin-6-yl)acetamide

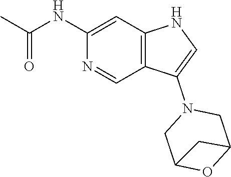 [SMILES: CC(=O)Nc1cc2[nH]cc(N3CC4CC(C3)O4)c2cn1]

To a solution of N-(3-(6-oxa-3-azabicyclo[3.1.1]heptan-3-yl)-1-(tetrahydro-2H-pyran-2-yl)-1H-pyrazolo[4,3-c]pyridin-6-yl)acetamide (Preparation 409, 200 mg, 0.56 mmol) in HFIP (3 mL) was added TFA (0.6 mL, 7.84 mmol) and the reaction mixture was stirred at 25° C. for 16 h. The mixture was adjusted to pH=7 by NH₃·H₂O then evaporated under reduced pressure to give N-(3-(6-oxa-3-azabicyclo[3.1.1]heptan-3-yl)-1H-pyrazolo[4,3-c]pyridin-6-yl)acetamide (200 mg, crude) as dark oil. LCMS m/z=274.0 [M+H]⁺.

### Preparation 411: 4-chloro-2-(2-fluoropropan-2-yl)-6-methylpyrimidine

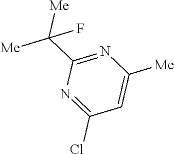 [SMILES: Cc1cc(Cl)nc(C(C)(C)F)n1]

To a solution of 4,6-dichloro-2-(2-fluoropropan-2-yl)pyrimidine (Preparation 206, 1 g, 4.78 mmol) in water (1 mL) and dioxane (5 mL) was added methyl boronic acid (286.4 mg, 4.78 mmol), Pd(dppf)Cl₂ (350 mg, 0.48 mmol) and K₂CO₃ (1.32 g, 9.57 mmol) and the mixture was stirred at 90° C. for 2 h under N₂. The mixture was diluted with water (20 mL) and extracted with EtOAc (25 mL×3). The combined organic layers were washed with brine (25 mL), dried over Na₂SO₄, filtered and concentrated to give a residue which was purified on silica gel column chromatography (PE/EtOAc=1/0 to 10/1) to give 4-chloro-2-(2-fluoropropan-2-yl)-6-methylpyrimidine (720 mg, 79.8% yield) as a yellow oil. ¹H NMR: (500 MHz, CDCl₃) δ ppm: 7.13 (s, 1H), 2.56 (s, 3H), 1.79 (s, 3H), 1.75 (s, 3H).

### Preparation 412: 4-chloro-6-ethyl-2-(2-fluoropropan-2-yl)pyrimidine

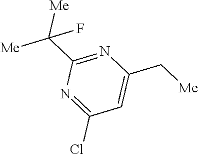 [SMILES: CCc1cc(Cl)nc(C(C)(C)F)n1]

4-Chloro-6-ethyl-2-(2-fluoropropan-2-yl)pyrimidine was obtained as a colorless oil, 486 mg, 50.1% yield, from 4,6-dichloro-2-(2-fluoropropan-2-yl)pyrimidine (Preparation 206) and ethylboronic acid following the procedure described in Preparation 411. ¹H NMR: (400 MHz, CDCl₃) δ ppm: 7.13 (s, 1H), 2.82 (q, J=7.6 Hz, 2H), 1.80 (s, 3H), 1.75 (s, 3H), 1.31 (t, J=7.6 Hz, 3H).

### Preparation 413: (Z)-3-amino-3-cyclopropylacrylamide

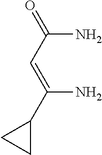

A solution of methyl 3-cyclopropyl-3-oxo-propanoate (20 g, 140.7 mmol) and NH₄OH (20 mL) in a teflon tank at 20° C., was heated to 50° C. for 24 h. The mixture reaction was concentrated under reduced pressure to give (Z)-3-amino-3-cyclopropylacrylamide (18 g, crude) as yellow oil. LCMS m/z=127.2 [M+H]⁺

### Preparation 414: 6-cyclopropyl-2-(1,1-difluoroethyl)pyrimidin-4-ol

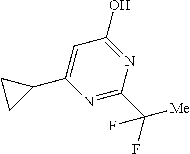 [SMILES: CC(F)(F)c1nc(O)cc(C2CC2)n1]

To a solution of (Z)-3-amino-3-cyclopropylacrylamide (Preparation 413, 17 g, 134.8 mmol) in EtOH (500 mL) was added sodium ethanolate (18.34 g, 269.5 mmol) and the mixture was stirred at 25° C. for 1 h. Ethyl 2,2-difluoropropanoate (27.92 g, 202.1 mmol) was added and the reaction was heated under reflux at 100° C. for 16 h. The reaction mixture was concentrated in vacuo to give 6-cyclopropyl-2-(1,1-difluoroethyl)pyrimidin-4-ol (40 g, crude) as yellow oil. LCMS m/z=201.1 [M+H]⁺.

### Preparation 415: 4-chloro-6-cyclopropyl-2-(1,1-difluoroethyl)pyrimidine

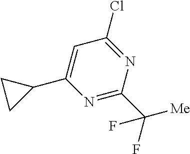 [SMILES: CC(F)(F)c1nc(Cl)cc(C2CC2)n1]

A mixture of 6-cyclopropyl-2-(1,1-difluoroethyl)pyrimidin-4-ol (Preparation 414, 10 g, 30.0 mmol) and POCl₃ (20.0 mL) was heated under reflux for 3 h. The cooled reaction was quenched with ice water and then extracted with DCM (200×2). The combined organic layer was dried over Na₂SO₄, filtered and concentrated in vacuo. The residue was purified by silica gel chromatography (EtOAc/PE=0/100 to 10/90) to give 4-chloro-6-cyclopropyl-2-(1,1-difluoroethyl)pyrimidine (900 mg, 11.7% yield) as yellow oil. LCMS m/z=218.9 [M+H]⁺

### Preparation 416: 2-bromo-6-(1,1-difluoroethyl)-4-methylpyridine

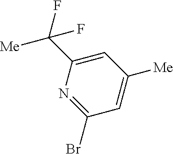 [SMILES: Cc1cc(Br)nc(C(C)(F)F)c1]

To a solution of 1-(6-bromo-4-methylpyridin-2-yl)ethan-1-one (360.5 mg, 1.68 mmol) in DCM (5.0 mL) was added DAST (3.7 g, 22.71 mmol) at 0° C. and the reaction stirred at 50° C. for 16 h. The mixture was poured into H₂O (30 mL) slowly and extracted with DCM (30 mL×3). The combined organic phase was washed with brine (30 mL×2), dried over Na₂SO₄ and filtered. The filtrate was concentrated under reduced pressure and the residue was purified by chromatography on silica gel (PE/EtOAc=20/1 to 5/1) to give 2-bromo-6-(1,1-difluoroethyl)-4-methylpyridine (280.6 mg, 70.6% yield) as colorless oil. ¹H NMR: (400 MHz, CDCl₃) δ ppm: 7.42 (s, 1H), 7.38 (s, 1H), 2.39 (s, 3H), 1.99 (t, J=18.8 Hz, 3H).

### Preparation 417: 2-(6-bromo-4-methoxypyridin-2-yl)propan-2-ol

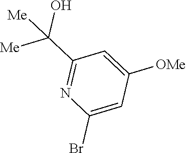 [SMILES: COc1cc(Br)nc(C(C)(C)O)c1]

To a solution of 2,6-dibromo-4-methoxypyridine (2.0 g, 7.49 mmol) in DCM (10 mL) was added n-BuLi (2.5 M, 3.0 mL) at −78° C. and the mixture was stirred for 30 min. Propan-2-one (435.2 mg, 7.49 mmol) was added and the reaction stirred for another 2 h at −78° C. The mixture was quenched with H₂O (20 mL) and extracted with EtOAc (30 mL×3). The combined organic layers were washed with brine (20 mL), dried over Na₂SO₄, filtered and concentrated under vacuum. The crude product was purified on silica gel column chromatography (PE/EtOAc=1/0 to 5/1) to give 2-(6-bromo-4-methoxypyridin-2-yl)propan-2-ol (1.3 g, 70.5% yield) as a white solid. LCMS m/z=248.0 [M+H]⁺

### Preparation 418: 1-(6-bromopyridin-2-yl)-2,2-difluorocyclopentan-1-ol

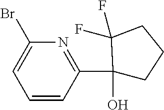 [SMILES: OC1(c2cccc(Br)n2)CCCC1(F)F]

To a solution of 2,6-dibromopyridine (300 mg, 1.27 mmol) in THF (10 mL) at −78° C. was added n-BuLi (2.5 M, 557 μL) and the mixture stirred for 30 min. 2,2-Difluorocyclopentanone (203 mg, 1.52 mmol) was added and the reaction mixture was stirred from −78° C. to rt overnight. The reaction was quenched with aq. NH₄Cl and extracted with EtOAc. The organic layer was dried and concentrated. The crude was purified by chromatography on silica gel (EtOAc/heptane 0/100 to 100/0) to give 1-(6-bromo-2-pyridyl)-2,2-difluoro-cyclopentanol (114 mg, 32% yield) as a colorless oil. LCMS m/z=278, 280 [M+H]⁺

### Preparation 419: 3-(6-bromopyrazin-2-yl)tetrahydrofuran-3-ol

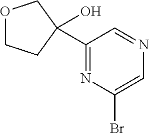 [SMILES: OC1(c2cncc(Br)n2)CCOC1]

3-(6-Bromopyrazin-2-yl)tetrahydrofuran-3-ol was obtained as a yellow oil, 3.5 g, 68% yield, from 2,6-dibromopyrazine and dihydrofuran-3(2H)-one, following a similar procedure to that described in Preparation 418. LCMS m/z=245 [M+H]⁺

### Preparation 420: 3-(3-bromophenyl)tetrahydrofuran-3-ol

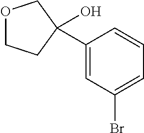 [SMILES: OC1(c2cccc(Br)c2)CCOC1]

3-(3-Bromophenyl)tetrahydrofuran-3-ol was obtained as a yellow oil, 6 g, 49%, from 1,3-dibromobenzene and dihydrofuran-3(2H)-one following a similar method to that described in Preparation 418. LCMS m/z=243 [M+H]⁺

### Preparation 421: 1-(6-bromopyridin-2-yl)-1-cyclopropylethan-1-ol

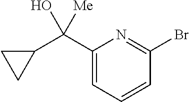 [SMILES: CC(O)(c1cccc(Br)n1)C1CC1]

To a solution of 1-(6-bromo-2-pyridyl)ethanone (1.08 g, 5.38 mmol) in THE (30 mL) at −78° C. was added bromo(cyclopropyl)magnesium (1 M, 7.0 mL). The mixture was stirred from −78° C. to rt over 2 h. The reaction was quenched with aq. NH₄Cl, extracted with EtOAc and the organic layer was separated, dried and concentrated. The crude was purified by chromatography on silica gel (0-100% EtOAc in heptane) to give 1-(6-bromopyridin-2-yl)-1-cyclopropylethan-1-ol (855 mg, 65% yield) as a colorless oil. LCMS m/z=242, 244 [M+H]⁺

### Preparation 422: 1-(2-bromo-5-methylthiazol-4-yl)ethan-1-ol

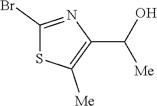

To a solution of 1-(2-bromo-5-methyl-thiazol-4-yl)ethanone (1.1 g, 5.0 mmol) in MeOH (16 mL) at 0° C. was added NaBH₄ (378 mg, 10.0 mmol) and the reaction was stirred at 0° C. for 1 h. The reaction mixture was concentrated in vacuo and the crude was purified by chromatography on silica gel (0-100% EtOAc in heptane) to give 1-(2-bromo-5-methylthiazol-4-yl)ethan-1-ol (725 mg, 65% yield) as a colorless oil. LCMS m/z=222 [M+H]⁺

### Preparation 423: 1-(2-bromothiazol-4-yl)ethan-1-ol

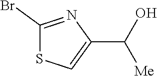

1-(2-Bromothiazol-4-yl)ethan-1-ol was obtained as a colorless oil, 404 mg, 80%, from 1-(2-bromothiazol-4-yl)ethanone, following the procedure described in Preparation 422. LCMS m/z=208 [M+H]⁺

### Preparation 424: 2-bromo-6-(cyclopropyl(methoxy)methyl)pyridine

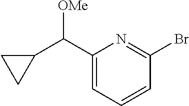 [SMILES: COC(c1cccc(Br)n1)C1CC1]

A solution of (6-bromo-2-pyridyl)-cyclopropyl-methanol (215 mg, 0.94 mmol) in THF (8 mL) was cooled to 0° C., NaH (57 mg, 1.41 mmol, 60% in mineral oil) was added and the reaction was stirred at 0° C. for 30 min. Iodomethane (669 mg, 4.71 mmol) was added and the reaction was stirred at rt overnight. Brine was added and the mixture was extracted with EtOAc. The organic layer was separated, dried and concentrated. The crude was purified by chromatography on silica gel (0-100% EtOAc in heptane) to give 2-bromo-6-(cyclopropyl(methoxy)methyl)pyridine (202 mg, 88% yield) as a colorless oil. LCMS m/z=242 [M+H]⁺

### Preparations 425 to 430

The compounds in the following table were prepared from the appropriate alcohol, following a similar procedure to that described in Preparation 424.

| Preparation | | Starting Material |
|---|---|---|
| No. | Name, Structure | (SM), Yield, data |
| 425 | 2-bromo-6-(1-cyclopropyl-1- methoxyethyl)pyridine 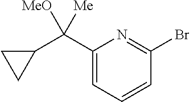 [SMILES: COC(C)(c1cccc(Br)n1)C1CC1] | SM: 1-(6-bromopyridin- 2-yl)-1-cyclopropylethan- 1-ol (Preparation 421) colorless oil, 28 mg, 13% LCMS m/z = 224, 226 [M − OMe] |
| 426 | 2-bromo-6-(2,2-difluoro-1- methoxycyclopentyl)pyridine 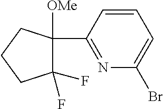 [SMILES: COC1(c2cccc(Br)n2)CCCC1(F)F] | SM: 1-(6-bromopyridin- 2-yl)-2,2- difluorocyclopentan-1-ol colorless oil, 98 mg, 84%, LCMS m/z = 292, 294 [M + H]⁺ |
| 427 | 2-bromo-6-(3- methoxytetrahydrofuran-3- yl)pyrazine 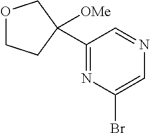 [SMILES: COC1(c2cncc(Br)n2)CCOC1] | SM: 3-(6-bromopyrazin-2- yl)tetrahydrofuran-3-ol (Preparation 419) 2.5 g, 67% Yield as yellow oil LCMS m/z = 259 [M + H]⁺ |
| 428 | 3-(3-bromophenyl)-3- methoxytetrahydrofuran 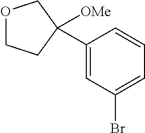 [SMILES: COC1(c2cccc(Br)c2)CCOC1] | SM: 3-(3-bromophenyl) tetrahydrofuran-3-ol 3.20 g, 50% Yield as yellow oil, LCMS m/z = 257 [M + H]⁺ |
| 429 | 2-bromo-4-(1-methoxyethyl)-5- methylthiazole 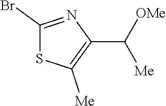 [SMILES: COC(C)c1nc(Br)sc1C] | SM: 1-(2-bromo-5- methylthiazol-4- yl)ethan-1-ol (Preparation 422) 241 mg, 90% yield as a colorless oil. LCMS m/z = 204, 206 [M − OMe] |
| 430 | 2-bromo-4-(1- methoxyethyl)thiazole 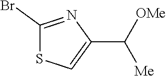 | SM: 1-(2-bromothiazol- 4-yl)ethan-1-ol (Preparation 423) 114 mg, 53% yield as a colorless oil. LCMS m/z = 222, 224 [M + H]⁺ |

### Preparation 431: 2,6-dibromo-4-(1,3-dioxolan-2-yl)pyridine

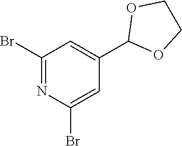 [SMILES: Brc1cc(C2OCCO2)cc(Br)n1]

A suspension of 2,6-dibromoisonicotinaldehyde (20 g, 74.9 mmol) in toluene (200 mL), ethane-1,2-diol (6.5 mL, 112.4 mmol) and 4-methylbenzenesulfonic acid hydrate (2.90 g, 1.75 mmol) was heated under reflux overnight using a Dean-Stark trap. The reaction was cooled, washed with water and brine. The organic layers were dried over Na₂SO₄, filtered, concentrated and purified by silica gel chromatography eluting with 20% EtOAc/hexane to give 2,6-dibromo-4-(1,3-dioxolan-2-yl)pyridine (13.0 g, 56% yield) as a white solid. LCMS m/z=308 [M+H]⁺

### Preparation 432: 3-(6-bromo-4-(1,3-dioxolan-2-yl)pyridin-2-yl)tetrahydrofuran-3-ol

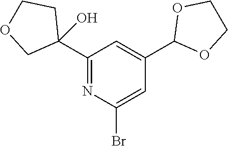 [SMILES: OC1(c2cc(C3OCCO3)cc(Br)n2)CCOC1]

n-BuLi in hexane (2.5 M, 36.7 mL, 91.8 mmol) was added dropwise to a solution of 2,6-dibromo-4-(1,3-dioxolan-2-yl)pyridine (Preparation 431, 13 g, 41.9 mmol) in DCM (200 mL) at −78° C. and the mixture was stirred for 1 h. Dihydrofuran-3(2H)-one (4.10 g, 47.4 mmol) in DCM (10 mL) was added, the reaction stirred for 1 h at −78° C., then allowed to warm to rt overnight. The reaction was quenched with NH₄Cl (aq. 50 mL), the aqueous layer was separated, and extracted with EtOAc (2×100 mL). The combined organic phase was washed with water and brine, dried over Na₂SO₄ and concentrated in vacuo. The crude was purified by chromatography on silica gel (30-50% EtOAc hexane) to give 3-(6-bromo-4-(1,3-dioxolan-2-yl)pyridin-2-yl)tetrahydrofuran-3-ol, (4.3 g, 33% yield) as a brown oil. LCMS m/z=316 [M+H]⁺

### Preparation 433: 2-bromo-4-(1,3-dioxolan-2-yl)-6-(3-methoxytetrahydrofuran-3-yl)pyridine

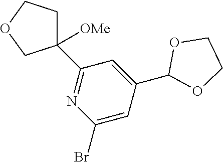 [SMILES: COC1(c2cc(C3OCCO3)cc(Br)n2)CCOC1]

2-Bromo-4-(1,3-dioxolan-2-yl)-6-(3-methoxytetrahydrofuran-3-yl)pyridine was obtained as a yellow oil, 3.0 g, 67% yield, from 3-(6-bromo-4-(1,3-dioxolan-2-yl)pyridin-2-yl)tetrahydrofuran-3-ol (Preparation 432), following the procedure described in Preparation 424. LCMS m/z=330 [M+H]⁺

### Preparation 434: 2-bromo-6-(3-methoxytetrahydrofuran-3-yl)isonicotinaldehyde

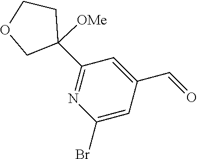 [SMILES: COC1(c2cc(C=O)cc(Br)n2)CCOC1]

5N HCl aqueous (18 mL, 91.0 mmol) was added to a solution of 2-bromo-4-(1,3-dioxolan-2-yl)-6-(3-methoxytetrahydrofuran-3-yl)pyridine (Preparation 433, 3.0 g, 9.10 mmol) in THE (30 mL) and the mixture was stirred at 50° C. for 3 h. The reaction was cooled to rt, the pH was adjusted to 7 and the mixture extracted with EtOAc (2×50 mL). The organic layer was concentrated and purified by silica gel chromatography eluting with 10-20% EtOAc in hexane to give 2-bromo-6-(3-methoxytetrahydrofuran-3-yl)isonicotinaldehyde, (2.3 g, 88% yield) as yellow oil. LCMS m/z=286 [M+H]⁺

### Preparation 435: 2-bromo-6-(2-fluoropropan-2-yl)-4-methoxypyridine

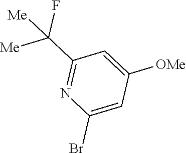 [SMILES: COc1cc(Br)nc(C(C)(C)F)c1]

To a solution of 2-(6-bromo-4-methoxypyridin-2-yl)propan-2-ol (1.3 g, 5.28 mmol) in DCM (10 mL) was added DAST (1.70 g, 10.56 mmol) and the reaction stirred at 25° C. for 16 h. The mixture was quenched with H₂O (20 mL) and extracted with EtOAc (20 mL×3). The combined organic layers were washed with brine (20 mL), dried over Na₂SO₄, filtered and concentrated in vacuo to give the crude, which was purified on silica gel column chromatography (PE/EtOAc=1/0 to 3/1) to give 2-bromo-6-(2-fluoropropan-2-yl)-4-methoxypyridine (1.0 g, 76.3% yield) as a white oil. LCMS m/z=250 [M+H]⁺

### Preparations 436 to 441

The compounds in the following table were prepared from the appropriate alcohol or ketone, following a similar procedure to that described in Preparation 435.

| Preparation | | Starting Material |
|---|---|---|
| No. | Name, Structure | (SM), Yield, data |
| 436 | 2-bromo-6-(1,1-difluoroethyl)-4- methoxypyridine 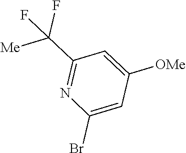 [SMILES: COc1cc(Br)nc(C(C)(F)F)c1] | SM: 1-(6-bromo-4- methoxypyridin-2- yl)ethan-1-one 420 mg, crude as yellow oil. LCMS m/z = 252.0 [M + H]⁺ |
| 437 | 2-bromo-6-(1,2- difluoroethyl)pyridine 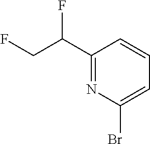 [SMILES: FCC(F)c1cccc(Br)n1] | SM: 1-(6-bromopyridin- 2-yl)ethane-1,2-diol 440 mg, 75.0% yield as a yellow oil. ¹H NMR (400 MHz, CDCl₃) δ ppm: 7.61-7.57 (m, 1H), 7.47-7.41 (m, 2H), 5.77- 5.71 (m, 1H), 4.95-4.64 (m, 2H) |
| 438 | 2-bromo-6-(1- fluorocyclobutyl)pyridine 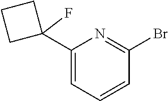 [SMILES: FC1(c2cccc(Br)n2)CCC1] | SM: 1-(6-bromopyridin- 2-yl)cyclobutan-1- colorless oil, 316 mg, 62% LCMS m/z = 230 [M + H]⁺ |
| 439 | tert-butyl 3-fluoro-3-(6- fluoropyridin-2-yl)azetidine-1- carboxylate 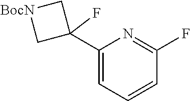 [SMILES: CC(C)(C)OC(=O)N1CC(F)(c2cccc(F)n2)C1] | SM: tert-butyl 3- (6-fluoro- 2-pyridyl)-3-hydroxy- azetidine-1-carboxylate 411 mg, 81% yield as a colorless oil LCMS m/z = 215 [M − tBu]⁺ |
| 440 | 2-chloro-6-(3- fluorotetrahydrofuran-3- yl)pyridine 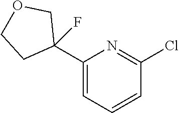 [SMILES: FC1(c2cccc(Cl)n2)CCOC1] | SM: 3-(6-chloro-2- pyridyl)tetrahydro- furan-3-ol 279 mg, 69% yield as a cololess oil. LCMS m/z = 202 [M + H]⁺ |
| 441 | 2-bromo-6-(1- fluoroethyl)pyridine 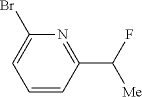 | SM: 1-(6-bromo-2- pyridyl)ethanol Light yellow solid, 0.94 g, 93%. LCMS m/z = 204 [M + H]⁺ |

### Preparation 442: 2-bromo-6-(1-cyclopropyl-1-fluoroethyl)pyridine

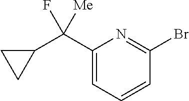 [SMILES: CC(F)(c1cccc(Br)n1)C1CC1]

A solution of 1-(6-bromopyridin-2-yl)-1-cyclopropylethan-1-ol (Preparation 421, 200 mg, 0.826 mmol) in DCM (5 mL) was cooled to −78° C. DAST (200 mg, 1.24 mmol) was added slowly, followed by N,N-diethylethanamine trihydrofluoride (133 mg, 0.826 mmol) and the reaction was stirred at 0° C. for 1 h. Aq. NaHCO₃ was added, the mixture was extracted with EtOAc and the organic layer was separated, dried and concentrated. The crude was purified by chromatography on silica gel (0-100% EtOAc in heptane) to give 2-bromo-6-(1-cyclopropyl-1-fluoro-ethyl)pyridine (72 mg, 35% yield) as a colorless oil. LCMS m/z=244, 246 [M+H]⁺

### Preparation 443: 2-bromo-6-(cyclopropylfluoromethyl)pyridine

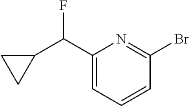 [SMILES: FC(c1cccc(Br)n1)C1CC1]

2-Bromo-6-(cyclopropylfluoromethyl)pyridine was obtained as a colorless oil, 186 mg, 36% from (6-bromo-2-pyridyl)-cyclopropyl-methanol, following the procedure described in Preparation 442. LCMS m/z=230 [M+H]⁺

### Preparation 444: 2-bromo-4-(1-fluoroethyl)thiazole

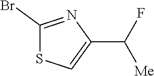

2-Bromo-4-(1-fluoroethyl)thiazole was obtained as a colorless oil, 86 mg, 42% from 1-(2-bromothiazol-4-yl)ethan-1-ol (Preparation 423), following the procedure described in Preparation 442. LCMS m/z=210, 212 [M+H]⁺.

### Preparation 445: 2-bromo-4-(1-fluoroethyl)-5-methylthiazole

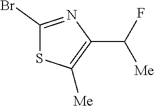

2-Bromo-4-(1-fluoroethyl)-5-methylthiazole was obtained as a colorless oil, 142 mg, 46% from 1-(2-bromo-5-methylthiazol-4-yl)ethan-1-ol (Preparation 422), following the procedure described in Preparation 442. LCMS m/z=224, 226 [M+H]⁺

### Preparation 446: 2-bromo-4-(difluoromethyl)-6-(3-methoxytetrahydrofuran-3-yl)pyridine

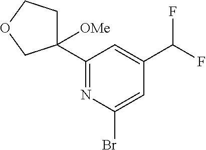 [SMILES: COC1(c2cc(C(F)F)cc(Br)n2)CCOC1]

To a solution of 2-bromo-6-(3-methoxytetrahydrofuran-3-yl)isonicotinaldehyde (Preparation 434, 2.3 g, 8.0 mmol) in DCM (30 mL), was added diethylaminosulfur trifluoride (5.30 mL, 40.0 mmol) at −78° C. The mixture was warmed up to rt and stirred for 2 h. The reaction mixture was carefully poured into ice water (40 mL) and NaHCO₃ solution, then extracted with DCM (3×30 mL). The combined organic layers were washed with NaHCO₃ solution, water (30 mL) and brine (30 mL), dried over Na₂SO₄ and concentrated. The crude was purified by column chromatography on silica gel (0-10% EtOAc in hexane) to give 2-bromo-4-(difluoromethyl)-6-(3-methoxytetrahydrofuran-3-yl)pyridine, (1.5 g, 60% yield) as yellow oil, LCMS m/z=308 [M+H]⁺

### Preparation 447: 2-chloro-6-(2-fluoropropan-2-yl)pyridine

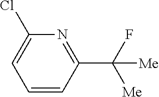 [SMILES: CC(C)(F)c1cccc(Cl)n1]

A solution of 2-(6-chloro-2-pyridyl)propan-2-ol (300 mg, 1.75 mmol) in DCM (12 mL) was cooled to −78° C. Deoxo-Fluor (4 M, 1.31 mL) was added slowly and the reaction warmed to rt overnight. The reaction mixture was cooled to 0° C. and aq.NaHCO₃ was added slowly. The organic layer was separated, dried and concentrated. The crude purified by chromatography on silica gel (0-40% EtOAc in heptane) to give 2-chloro-6-(2-fluoropropan-2-yl)pyridine (220 mg, 65% yield) as a colorless oil. LCMS m/z=174 [M+H]⁺

### Preparation 448: 2-bromo-6-(2,2-difluoroethyl)pyridine

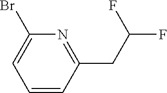 [SMILES: FC(F)Cc1cccc(Br)n1]

A cold solution of 2-(6-bromo-2-pyridyl)ethanol (1.00 g, 4.95 mmol) in DCM (30 mL) was added to a solution of Dess-Martin periodinane (2.52 g, 5.94 mmol) in one portion at 0° C. and the solution stirred for 1 h at 0° C. Deoxo-Fluor (2.2 M, 9.12 mL) was added and the reaction was stirred at 0° C. for an additional 1 h. The mixture was carefully poured into saturated NaHCO₃ and the organic layer was separated. The aqueous layer was extracted with EtOAc (3×), the combined organic layer was washed with water, then brine, and then dried over MgSO4 and concentrated. The crude was purified by silica gel column (10-50% EtOAc/heptane) to give 2-bromo-6-(2,2-difluoroethyl)pyridine as a yellowish oil (101 mg). LCMS m/z=221.9, 223.9 [M+H]⁺

### Preparation 449: 4-(benzyloxy)-2-(1,1-difluoroethyl)-6-(prop-1-en-2-yl)pyrimidine

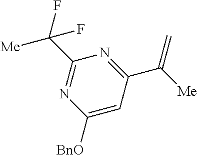 [SMILES: C=C(C)c1cc(OCc2ccccc2)nc(C(C)(F)F)n1]

Step 1: To a solution of 4,6-dichloro-2-(1,1-difluoroethyl)pyrimidine (5.0 g, 23.47 mmol) in THF (40 mL) was added NaH (1.9 g, 46.95 mmol, 60% purity) and BnOH (2.7 g, 24.55 mmol) and the reaction stirred at 25° C. for 1 h. The reaction mixture was concentrated in vacuo to give crude 4-(benzyloxy)-6-chloro-2-(1,1-difluoroethyl)pyrimidine.

Step 2: To 4-(benzyloxy)-6-chloro-2-(1,1-difluoroethyl)pyrimidine (6.7 g, 23.5 mmol) was added water (10 mL), then 4,4,5,5-tetramethyl-2-(prop-1-en-2-yl)-1,3,2-dioxaborolane (7.9 g, 46.9 mmol), K₂CO₃ (10.0 g, 72.4 mmol) and Pd(dppf)Cl₂ (1.7 g, 2.35 mmol) and the reaction stirred at 70° C. for 2 h under N₂. The mixture was quenched with H₂O (100 mL) and extracted with EtOAc (50 mL×3). The combined organic layers were washed with brine (100 mL), dried over Na₂SO₄, filtered and concentrated under vacuum to give the crude, which was purified on silica gel column chromatography (PE/EtOAc=1/0 to 10/1) to give 4-(benzyloxy)-2-(1,1-difluoroethyl)-6-(prop-1-en-2-yl)pyrimidine (6.0 g, yield: 88%, two steps) as a white solid. ¹H NMR: (400 MHz, DMSO-d₆) δ ppm: 7.52-7.49 (m, 2H), 7.40-7.35 (m, 3H), 7.17 (s, 1H), 6.26 (s, 1H), 5.52-5.51 (m, 1H), 5.47 (s, 2H), 5.47-5.41 (s, 1H), 2.11-1.99 (m, 3H), 1.73-1.72 (m, 3H).

### Preparation 450: 2-(1,1-difluoroethyl)-6-isopropylpyrimidin-4-ol

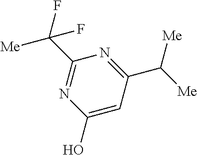 [SMILES: CC(C)c1cc(O)nc(C(C)(F)F)n1]

To a solution of 4-(benzyloxy)-2-(1,1-difluoroethyl)-6-(prop-1-en-2-yl)pyrimidine (Preparation 449, 6.0 g, 20.67 mmol) in EtOH (60 mL) was added Pd/C (4.4 g, 4.13 mmol, 10% purity) and the reaction mixture was stirred at 65° C. under H₂ at 15 Psi for 4 h. The mixture was quenched with H₂O (100 mL) and extracted with EtOAc (50 mL×3). The combined organic layers were washed with brine (100 mL), dried over Na₂SO₄, filtered and concentrated under vacuum to give the crude, which was purified on silica gel column chromatography (PE/EtOAc=1/0 to 1/1) to give 2-(1,1-difluoroethyl)-6-isopropylpyrimidin-4-ol (3.0 g, 72.5% yield) as a white solid. ¹H NMR: (400 MHz, DMSO-d₆) δ ppm 12.71 (br s, 1H), 6.46 (br s, 1H), 2.87-2.79 (m, 1H), 2.03-1.91 (m, 3H), 1.17 (d, J=6.8 Hz, 6H).

### Preparation 451: 4-chloro-2-(1,1-difluoroethyl)-6-isopropylpyrimidine

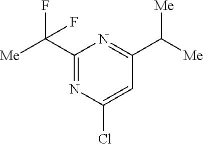 [SMILES: CC(C)c1cc(Cl)nc(C(C)(F)F)n1]

To a solution of 2-(1,1-difluoroethyl)-6-isopropylpyrimidin-4-ol (Preparation 450, 3.0 g, 15.0 mmol) was added POCl₃ (8.3 g, 54.2 mmol) and the reaction was stirred at 100° C. for 1 h. The mixture was concentrated, diluted with DCM (100 mL) and aq.NaHCO₃ (200 mL) slowly added. The mixture was stirred at 25° C. for 1 h, the layers separated and the aqueous extracted with DCM (20 mL×3). The combined organic layers were washed with brine (50 mL), dried over Na₂SO₄, filtered and concentrated to give 4-chloro-2-(1,1-difluoroethyl)-6-isopropylpyrimidine (3.1 g, 93.7% yield) as a yellow oil. ¹H NMR (400 MHz, CDCl₃) δ ppm: 7.20 (d, J=2.4 Hz, 1H), 3.07-2.98 (m, 1H), 2.03-1.94 (m, 3H), 1.27-1.25 (m, 6H).

### Preparation 452: 2,4-dichloro-5-fluoro-6-methylpyrimidine

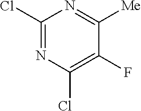

To a solution of 2,4-dichloro-5-fluoropyrimidine (15.0 g, 89.84 mmol) in DME (150 mL) was slowly added MeMgBr (3 M, 44.9 mL) at 0° C. under N₂ and the mixture was stirred at 15° C. for 1 h. TEA (9.1 g, 89.84 mmol) in THF (10 mL) was added to the solution at 0° C. After 5 mins, I₂ (22.8 g, 89.84 mmol) in THF (16 mL) was slowly added and the reaction stirred at 15° C. for 2 h. The mixture was quenched with H₂O (200 mL), and the pH adjusted to pH=1 with 5N HCl. The mixture was extracted with EtOAc (150 mL×3), the combined organic phase was washed with brine (300 mL), dried with Na₂SO₄, filtered and concentrated in vacuo. The crude was purified by column chromatography (PE/EtOAc=0/1 to 10/1) on silica gel to give 2,4-dichloro-5-fluoro-6-methylpyrimidine (9.3 g, 57.0% yield) as yellow oil. ¹H NMR (500 MHz, CDCl₃) δ ppm: 2.56 (d, J=2.5 Hz, 3H).

### Preparation 453: 4-(benzyloxy)-2-chloro-5-fluoro-6-methylpyrimidine

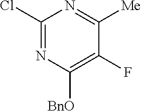 [SMILES: Cc1nc(Cl)nc(OCc2ccccc2)c1F]

A solution of BnOH (5.8 g, 53.87 mmol) and t-BuONa (5.2 g, 53.87 mmol) in toluene (100 mL) was stirred at 0° C. for 10 mins, then slowly added to 2,4-dichloro-5-fluoro-6-methylpyrimidine (Preparation 452, 9.8 g, 53.87 mmol). The resulting mixture was stirred at 20° C. for 1 h. The reaction was treated with H₂O (150 mL) and extracted with EtOAc (100 mL×3). The combined organic phase was washed with brine (200 mL), dried over Na₂SO₄, filtered, concentrated, then purified by column chromatography (PE/EtOAc=20/1 to 5/1) on silica gel to give 4-(benzyloxy)-2-chloro-5-fluoro-6-methylpyrimidine (7.9 g, 58.2% yield) as colorless oil.

### Preparation 454: 4-(benzyloxy)-2-chloro-5-fluoropyrimidine

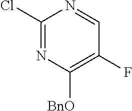 [SMILES: Fc1cnc(Cl)nc1OCc1ccccc1]

4-(Benzyloxy)-2-chloro-5-fluoropyrimidine was obtained as a white solid, 12.5 g, 87.5%, from 2,4-dichloro-5-fluoropyrimidine, following the procedure described in Preparation 453. 1H NMR: (500 MHz, CDCl₃) δ ppm: 8.21 (s, 1H), 7.49-7.42 (m, 2H), 7.41-7.38 (m, 3H), 5.51 (s, 2H).

### Preparation 455: methyl 4-(benzyloxy)-5-fluoro-6-methylpyrimidine-2-carboxylate

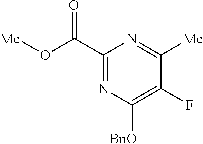 [SMILES: COC(=O)c1nc(C)c(F)c(OCc2ccccc2)n1]

To a solution of 4-(benzyloxy)-2-chloro-5-fluoro-6-methylpyrimidine (Preparation 453, 7.9 g, 31.35 mmol) in MeOH (80 mL) was added TEA (6.4 g, 62.69 mmol) and Pd(dppf)Cl₂ (458.7 mg, 0.63 mmol) and the reaction was stirred at 80° C. for 16 h under CO (50 psi). The mixture was concentrated in vacuo and was purified by chromatography (PE/EtOAc=20/1 to 5/1) on silica gel to give methyl 4-(benzyloxy)-5-fluoro-6-methylpyrimidine-2-carboxylate (3.1 g, 35.8% yield) as yellow oil. 1H NMR: (400 MHz, CDCl₃) δ ppm: 7.53-7.51 (m, 2H), 7.40-7.35 (m, 3H), 5.58 (s, 2H), 4.03 (s, 3H), 2.55 (d, J=2.8 Hz, 3H).

### Preparation 456: methyl 4-(benzyloxy)-5-fluoropyrimidine-2-carboxylate

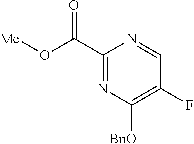 [SMILES: COC(=O)c1ncc(F)c(OCc2ccccc2)n1]

Methyl 4-(benzyloxy)-5-fluoropyrimidine-2-carboxylate was obtained as a white solid, 12 g, 42% yield, from 4-(benzyloxy)-2-chloro-5-fluoropyrimidine (Preparation 454), following the procedure described in Preparation 455. 1H NMR (400 MHz, CDCl₃) δ ppm: 8.44 (d, J=2.0 Hz, 1H), 7.53-7.39 (m, 2H), 7.38-7.35 (m, 3H), 5.61 (s, 2H), 4.03 (s, 3H).

### Preparation 457: 1-(4-(benzyloxy)-5-fluoro-6-methylpyrimidin-2-yl)ethan-1-one

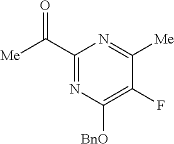 [SMILES: CC(=O)c1nc(C)c(F)c(OCc2ccccc2)n1]

To a solution of methyl 4-(benzyloxy)-5-fluoro-6-methylpyrimidine-2-carboxylate (Preparation 455, 3.1 g, 11.22 mmol) in THF (30 mL) was slowly added dropwise MeMgBr (3 M, 3.8 mL) at −70° C. under N₂. The mixture was stirred at −70° C. for 1.5 h. The reaction was quenched with sat. aq.NH₄Cl (30 mL), treated with H₂O (20 mL) and extracted with EtOAc (40 mL×3). The organic phase was washed with brine (50 mL), dried with Na₂SO₄, filtered, concentrated, then purified by column chromatography (PE/EtOAc=15/1 to 3/1) on silica gel to give 1-(4-(benzyloxy)-5-fluoro-6-methylpyrimidin-2-yl)ethan-1-one (1.6 g, 54.8% yield) as yellow oil. ¹H NMR: (400 MHz, CDCl₃) δ ppm: 7.51-7.49 (m, 2H), 7.40-7.36 (m, 3H), 5.57 (s, 2H), 2.71 (s, 3H), 2.54 (d, J=3.2 Hz, 3H).

### Preparation 458: 1-(4-(benzyloxy)-5-fluoropyrimidin-2-yl)ethan-1-one

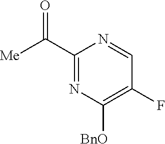 [SMILES: CC(=O)c1ncc(F)c(OCc2ccccc2)n1]

1-(4-(Benzyloxy)-5-fluoropyrimidin-2-yl)ethan-1-one was obtained as a white solid, 7.3 g, 64.8% yield, from methyl 4-(benzyloxy)-5-fluoropyrimidine-2-carboxylate (Preparation 456), following the procedure described in Preparation 457. 1H NMR (400 MHz, CDCl₃) δ ppm: 8.45 (d, J=2.0 Hz, 1H), 7.52-7.49 (m, 2H), 7.42-7.37 (m, 3H), 5.60 (s, 2H), 2.73 (s, 3H).

### Preparation 459: 4-(benzyloxy)-2-(1,1-difluoroethyl)-5-fluoro-6-methylpyrimidine

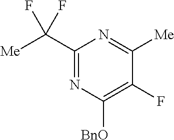 [SMILES: Cc1nc(C(C)(F)F)nc(OCc2ccccc2)c1F]

To a solution of 1-(4-(benzyloxy)-5-fluoro-6-methylpyrimidin-2-yl)ethan-1-one (Preparation 457, 1.5 g, 5.76 mmol) in DCM (4 mL) was added DAST (4.65 g, 28.82 mmol) and the reaction stirred at 25° C. for 16 h. The mixture was treated with H₂O (10 mL) and extracted with DCM (10 mL×3). The combined organic phase was washed with brine (20 mL), dried with Na₂SO₄, filtered, concentrated, then purified by column chromatography (PE/EtOAc=15/1 to 3/1) on silica gel to give 4-(benzyloxy)-2-(1,1-difluoroethyl)-5-fluoro-6-methylpyrimidine (1.6 g, 95.9% yield) as yellow oil. ¹H NMR: (500 MHz, CDCl₃) δ ppm: 7.48-7.46 (m, 2H), 7.38-7.32 (m, 3H), 5.51 (s, 2H), 2.48 (d, J=3.0 Hz, 3H), 1.98 (t, J=18.0 Hz, 3H).

### Preparation 460: 4-(benzyloxy)-2-(1,1-difluoroethyl)-5-fluoropyrimidine

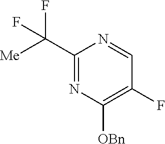 [SMILES: CC(F)(F)c1ncc(F)c(OCc2ccccc2)n1]

4-(Benzyloxy)-2-(1,1-difluoroethyl)-5-fluoropyrimidine was obtained as a colorless oil, 6.7 g, 84% from 1-(4-(benzyloxy)-5-fluoropyrimidin-2-yl)ethan-1-one (Preparation 458), following the procedure described in Preparation 459. 1H NMR: (400 MHz, CDCl₃) δ ppm: 8.38 (d, J=2.4 Hz, 1H), 7.51-7.40 (m, 2H), 7.39-7.36 (m, 3H), 5.56 (s, 2H), 2.02 (t, J=18.4 Hz, 3H).

### Preparation 461: 2-(1,1-difluoroethyl)-5-fluoro-6-methylpyrimidin-4-ol

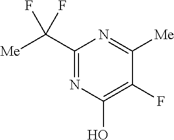 [SMILES: Cc1nc(C(C)(F)F)nc(O)c1F]

A solution of 4-(benzyloxy)-2-(1,1-difluoroethyl)-5-fluoro-6-methylpyrimidine (Preparation 459, 1.6 g, 5.53 mmol) in TFA (3 mL) was stirred at 25° C. for 16 h and a further 3 h at 100° C. The mixture was concentrated under reduced pressure to give the residue, which was purified by column chromatography (PE/EtOAc=15/1 to 3/1) on silica gel to give 2-(1,1-difluoroethyl)-5-fluoro-6-methylpyrimidin-4-ol (780.0 mg, 73.5% yield) as colorless oil. 1H NMR: (500 MHz, CDCl₃) δ ppm: 2.40 (d, J=3.5 Hz, 3H), 2.02 (t, J=18.5 Hz, 3H).

### Preparation 462: 2-(1,1-difluoroethyl)-5-fluoropyrimidin-4-ol

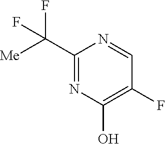 [SMILES: CC(F)(F)c1ncc(F)c(O)n1]

2-(1,1-Difluoroethyl)-5-fluoropyrimidin-4-ol was obtained as a white solid, 3.2 g, 72.1% yield from 4-(benzyloxy)-2-(1,1-difluoroethyl)-5-fluoropyrimidine (Preparation 460), following a similar procedure to that described in Preparation 461. 1H NMR: (400 MHz, CDCl₃) δ ppm: 11.61 (br s, 1H), 7.93 (d, J=2.4 Hz, 1H), 2.03 (t, J=18.8 Hz, 3H).

### Preparation 463: 4-chloro-2-(1,1-difluoroethyl)-5-fluoro-6-methylpyrimidine

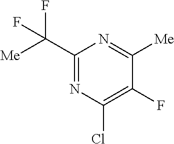 [SMILES: Cc1nc(C(C)(F)F)nc(Cl)c1F]

A solution of 2-(1,1-difluoroethyl)-5-fluoro-6-methylpyrimidin-4-ol (Preparation 461, 3.1 g, 16.24 mmol) in POCl₃ (24.0 mL, 257.5 mmol) was stirred at 100° C. for 30 mins. The mixture was concentrated under reduced pressure to give the residue, which was added to H₂O (15 mL), extracted with DCM (10×3 mL), the combined organic phase was dried over anhydrous Na₂SO₄, filtered and concentrated, then purified by column chromatography (PE/EtOAc=15/1 to 3/1) on silica gel to give 4-chloro-2-(1,1-difluoroethyl)-5-fluoro-6-methylpyrimidine (3.1 g, 90.7% yield) as colorless oil. LCMS m/z=211.0 [M+H]⁺

### Preparation 464: 4-chloro-2-(1,1-difluoroethyl)-5-fluoropyrimidine

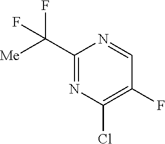 [SMILES: CC(F)(F)c1ncc(F)c(Cl)n1]

4-Chloro-2-(1,1-difluoroethyl)-5-fluoropyrimidine was obtained as a colorless oil, 2.3 g, 65.1% from 2-(1,1-difluoroethyl)-5-fluoropyrimidin-4-ol (Preparation 462), following the procedure described in Preparation 463. LCMS m/z=197.1 [M+H]⁺

### Preparation 465: 2-oxabicyclo[2.1.1]hexane-4-carboxamide

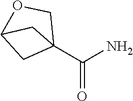

To a solution of 2-oxabicyclo[2.1.1]hexane-4-carboxylic acid (2 g, 15.61 mmol) in DCM (30 mL) under N₂ was added oxalyl chloride (2 M, 9.4 mL) and DMF (114.09 mg, 1.56 mmol) and the solution stirred at rt for 1 h. The reaction mixture was concentrated in vacuo, the residue was dissolved in DCM (20 mL) and cooled to 0° C. Ammonia (7 M in MeOH, 33.45 mL) was slowly added and the mixture was stirred at rt for 2 h. The resulting solid was filtered off and rinsed with DCM. The filtrate was concentrated, diluted with MeCN, the solid formed was collected by filtration and rinsed with MeCN to give 2-oxabicyclo[2.1.1]hexane-4-carboxamide as a white solid, 1.86 g, 93% yield. NMR (MeOD-d₄, 400 MHz) δ 4.50 (t, 1H, J=1.1 Hz), 3.81 (s, 2H), 2.12 (td, 2H, J=1.3, 4.9 Hz), 1.7-1.9 (m, 2H).

### Preparation 466: 2-(2-oxabicyclo[2.1.1]hexan-4-yl)-6-methylpyrimidin-4(3H)-one

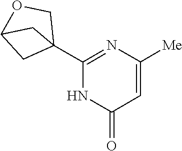 [SMILES: Cc1cc(=O)[nH]c(C23COC(C2)C3)n1]

A solution of NaOMe (4.88 M, 2.56 mL, 25 wt % in MeOH) in n-butanol (2 mL) was heated to 105° C. in a sealed vial (poked with a needle open to air) while the solvent was removed. A solution of methyl (E)-3-aminobut-2-enoate (360 mg, 3.13 mmol) and 2-oxabicyclo[2.1.1]hexane-4-carboxamide (Preparation 465, 1 g, 7.87 mmol) in n-butanol (6.0 mL) and MeOH (6.0 mL) was then added, and after the addition, the reaction temperature was raised to 110° C. and stirring continued for 2 h. conc. HCl was added to acidify the mixture, the resulting solid filtered off and rinsed with MeOH. The filtrate was concentrated under reduced pressure, the residue was diluted with MeCN, the resulting solid was filtered off and rinsed with MeCN. The filtrate was concentrated, the residue diluted with MeCN, the solid formed was collected by filtration, and rinsed with cold MeCN to give 2-(2-oxabicyclo[2.1.1]hexan-4-yl)-6-methylpyrimidin-4(3H)-one, as a white solid. LCMS m/z=193.1 [M+H]⁺

### Preparation 467: 2-(2-oxabicyclo[2.1.1]hexan-4-yl)-6-ethylpyrimidin-4(3H)-one

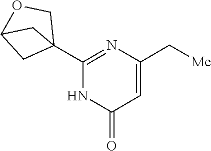 [SMILES: CCc1cc(=O)[nH]c(C23COC(C2)C3)n1]

2-(2-Oxabicyclo[2.1.1]hexan-4-yl)-6-ethylpyrimidin-4(3H)-one was obtained as a solid, from methyl (E)-3-aminopent-2-enoate, sodium ethoxide and 2-oxabicyclo[2.1.1]hexane-4-carboxamide (Preparation 465), following a similar procedure to that described in Preparation 466. LCMS m/z=207.1 [M+H]⁺

### Preparation 468: 2-(2-oxabicyclo[2.1.1]hexan-4-yl)-4-chloro-6-methylpyrimidine

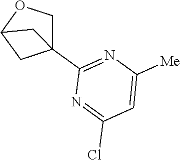 [SMILES: Cc1cc(Cl)nc(C23COC(C2)C3)n1]

A mixture of 2-(2-oxabicyclo[2.1.1]hexan-4-yl)-6-methylpyrimidin-4(3H)-one (Preparation 466, 170 mg, 0.884 mmol), DMF (34 μL) and POCl₃ (4.42 mmol, 412 μL) in DCM (3 mL) was stirred at rt for 2 h. The reaction was concentrated under reduced pressure, the residue diluted with EtOAc, then ice and neutralized with NaHCO₃ (aq). The layers were separated, the aqueous layer was extracted with EtOAc (2×). The combined organic layer was dried and concentrated to give 4-chloro-6-methyl-2-(2-oxabicyclo[2.1.1]hexan-4-yl)pyrimidine (149 mg, 79% yield) as a yellowish oil. LCMS m/z=211.1 [M+H]⁺

### Preparation 469: 2-(2-oxabicyclo[2.1.1]hexan-4-yl)-4-chloro-6-ethylpyrimidine

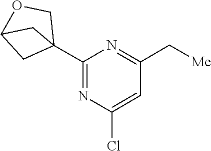 [SMILES: CCc1cc(Cl)nc(C23COC(C2)C3)n1]

2-(2-Oxabicyclo[2.1.1]hexan-4-yl)-4-chloro-6-ethylpyrimidine was obtained as a colorless oil, 178 mg, 56% yield from 2-(2-oxabicyclo[2.1.1]hexan-4-yl)-6-ethylpyrimidin-4(3H)-one (Preparation 467), following the procedure described in Preparation 468. LCMS m/z=225.1 [M+H]⁺

### Preparation 470: 4-bromo-2-(1,1-difluoroethyl)-1-methyl-1H-imidazole

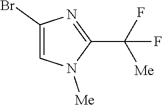 [SMILES: Cn1cc(Br)nc1C(C)(F)F]

To a mixture of 4-bromo-2-(1,1-difluoroethyl)-1H-imidazole (200 mg, 0.948 mmol) and K₂CO₃ (262 mg, 1.90 mmol) in DMF (2 mL) was added iodomethane (413 μL, 6.63 mmol) and the reaction was stirred at rt for 16 h. The mixture was diluted with EtOAc and washed with water (3×). The organic layer was separated, dried and concentrated. The solid formed was washed with MeCN to give 4-bromo-2-(1,1-difluoroethyl)-1-methyl-imidazole (137 mg, 64% yield) as a white powder. LCMS m/z=225 [M+H]⁺

### Preparation 471: 4-chloro-2-(1,1-difluoroethyl)-6-isopropoxypyrimidine

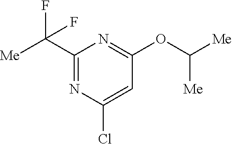 [SMILES: CC(C)Oc1cc(Cl)nc(C(C)(F)F)n1]

To a solution of 4,6-dichloro-2-(1,1-difluoroethyl)pyrimidine (200 mg, 0.94 mmol) in THF (5 mL) was added sodium isopropoxide (462.4 mg, 1.13 mmol, 20% purity) and the reaction was stirred at 25° C. for 3 h. The mixture was concentrated and the residue purified by prep-TLC (PE/EtOAc=10/1) to afford 4-chloro-2-(1,1-difluoroethyl)-6-isopropoxypyrimidine (87 mg, 39.2% yield) as a yellow oil. ¹H NMR (500 MHz, CDCl₃) δ ppm: 6.73 (s, 1H), 5.47-5.39 (m, 1H), 2.00 (t, J=18.5 Hz, 3H), 1.38 (d, J=6.5 Hz, 6H).

### Preparation 472: 4-chloro-2-(1,1-difluoroethyl)-6-ethoxypyrimidine

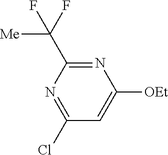 [SMILES: CCOc1cc(Cl)nc(C(C)(F)F)n1]

To a solution of 4,6-dichloro-2-(1,1-difluoroethyl)pyrimidine (200 mg, 0.94 mmol) in EtOH (2 mL) was added EtONa (76.67 mg, 1.13 mmol) and the mixture was stirred at 45° C. for 2 h. The mixture was diluted with water (20 mL) and extracted with EtOAc (25 mL×3). The combined organic layers were washed with brine (25 mL), dried over Na₂SO₄, filtered and concentrated to give a residue which was purified on silica gel column chromatography (PE/EtOAc=1/0 to 5/1) to give 4-chloro-2-(1,1-difluoroethyl)-6-ethoxypyrimidine (110 mg, 52.6% yield) as a yellow solid. ¹H NMR (500 MHz, CDCl₃) δ ppm: 6.77 (s, 1H), 4.50 (q, J=7.0 Hz, 2H), 2.04-1.97 (m, 3H), 1.41 (t, J=7.0 Hz, 3H).

### Preparation 473: 4-chloro-6-cyclopropoxy-2-(1,1-difluoroethyl)pyrimidine

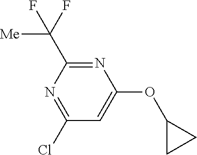 [SMILES: CC(F)(F)c1nc(Cl)cc(OC2CC2)n1]

To a degassed solution of cyclopropanol (49.6 mg, 0.85 mmol) in THF (2 mL) was added NaH (41.0 mg, 1.02 mmol, 60% purity) and the mixture was stirred at 0° C. for 0.5 h. 4,6-Dichloro-2-(1,1-difluoroethyl)pyrimidine (200 mg, 0.94 mmol) was added and the reaction stirred at 25° C. for 2 h. The mixture was poured into H₂O (20 mL) and extracted with EtOAc (20 mL×3). The combined organic phase was washed with brine (10 mL×2), dried over Na₂SO₄ and filtered. The filtrate was concentrated under reduced pressure and the residue was purified by chromatography on silica gel (PE/EtOAc=20/1 to 5/1) to give 4-chloro-6-cyclopropoxy-2-(1,1-difluoroethyl)pyrimidine, 137 mg, 68.4% yield, as colorless oil. LCMS m/z=235.1 [M+H]⁺

### Preparation 474: 4-chloro-2-(1,1-difluoroethyl)-6-(2-methoxyethoxy)pyrimidine

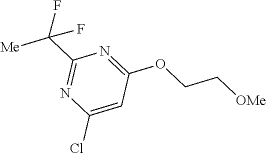 [SMILES: COCCOc1cc(Cl)nc(C(C)(F)F)n1]

To a solution of 4,6-dichloro-2-(1,1-difluoroethyl)pyrimidine (200 mg, 0.939 mmol), Cs₂CO₃ (917.8 mg, 2.82 mmol) in DMF (5 mL) was added 2-methoxyethan-1-ol (78.6 mg, 1.03 mmol) and the reaction stirred at 25° C. for 1 h. The reaction mixture was quenched by the addition of water and extracted with EtOAc (40 mL×3). The combined organic layers were washed with H₂O and brine (40 mL), dried over Na₂SO₄, filtered and concentrated under reduced pressure. The residue was purified on silica gel column chromatography (PE/EtOAc=10/1) to give 4-chloro-2-(1,1-difluoroethyl)-6-(2-methoxyethoxy)pyrimidine (180 mg, 75.9% yield) as a yellow oil. LCMS m/z=253.0 [M+H]⁺

### Preparation 475: 4-chloro-2-(1,1-difluoroethyl)-6-((1r,3r)-3-methoxycyclobutoxy)pyrimidine

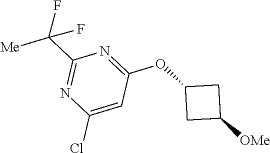 [SMILES: CO[C@H]1C[C@H](Oc2cc(Cl)nc(C(C)(F)F)n2)C1]

4-Chloro-2-(1,1-difluoroethyl)-6-((1r,3r)-3-methoxycyclobutoxy)pyrimidine was obtained as a yellow oil, 450 mg, 86.0% from 4,6-dichloro-2-(1,1-difluoroethyl)pyrimidine and (1r,3r)-3-methoxycyclobutan-1-ol, following a similar procedure to that described in Preparation 474. LCMS m/z=278.9 [M+H]⁺

### Preparation 476: 2-(1,1-difluoroethyl)-4-((1s,3s)-3-methoxycyclobutoxy)pyridine

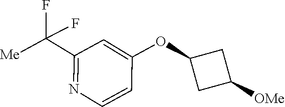 [SMILES: CO[C@H]1C[C@@H](Oc2ccnc(C(C)(F)F)c2)C1]

To a solution of 4-chloro-6-(1,1-difluoroethyl)pyridine (3.55 g, 20.0 mmol) and (1s,3s)-3-methoxycyclobutan-1-ol (1.7 g, 16.65 mmol) in DMF (35 mL) was added NaH (865.6 mg, 21.64 mmol, 60% purity) and the reaction mixture was stirred at 60° C. for 16 h. The reaction was quenched with water (50 mL) and extracted with EtOAc (3×50 mL). The combined organic layer was washed with brine (3×100 mL), dried over Na₂SO₄ and filtered. The filtrate was concentrated in vacuo and the residue was purified by column chromatography on silica gel (PE/EtOAc=10/1 to 5/1) to give 2-(1,1-difluoroethyl)-4-((1s,3s)-3-methoxycyclobutoxy)pyridine (3.7 g, 91.4% yield) as a colorless oil. 1H NMR (400 MHz, CDCl₃) δ ppm 8.43 (d, J=5.6 Hz, 1H), 7.06 (d, J=2.4 Hz, 1H), 6.76 (dd, J=2.4, 5.6 Hz, 1H), 4.44-4.37 (m, 1H), 3.73-3.66 (m, 1H), 3.28 (s, 3H), 2.96-2.59 (m, 2H), 2.17-2.14 (m, 2H), 1.99 (t, J=22.4 Hz, 3H).

### Preparation 477: 2-(1,1-difluoroethyl)-4-((1r,3r)-3-methoxycyclobutoxy)pyridine

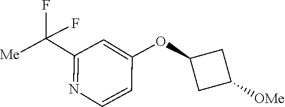 [SMILES: CO[C@H]1C[C@H](Oc2ccnc(C(C)(F)F)c2)C1]

2-(1,1-Difluoroethyl)-4-((1r,3r)-3-methoxycyclobutoxy)pyridine was obtained as a colorless oil, 5 g, 75% yield from 4-chloro-6-(1,1-difluoroethyl)pyridine and (1r,3r)-3-methoxycyclobutan-1-ol, following the procedure described in Preparation 476. 1H NMR (400 MHz, CDCl₃) δ ppm 8.43 (d, J=5.6 Hz, 1H), 7.04 (d, J=1.8 Hz, 6.4 Hz, 1H), 6.73 (dd, J=2.4, 5.6 Hz, 1H), 4.94-4.88 (m, 1H), 4.20-4.09 (m, 1H), 3.28 (s, 3H), 2.54-2.38 (m, 4H), 1.99 (t, J=18.8 Hz, 3H).

### Preparation 478: 2-(1,1-difluoroethyl)-4-((1s,3s)-3-methoxycyclobutoxy)pyridine 1-oxide

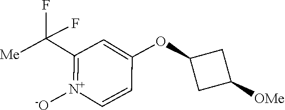

To a solution of 2-(1,1-difluoroethyl)-4-((1s,3s)-3-methoxycyclobutoxy)pyridine (Preparation 476, 3.70 g, 15.21 mmol) in DCM (60 mL) was added m-CPBA (3.87 g, 19.77 mmol, 80% purity) and the reaction mixture was stirred at 25° C. for 16 h. The reaction was quenched with sat.Na₂SO₃ (100 mL) and extracted with EtOAc (3×50 mL). The combined organic layer was dried over Na₂SO₄, filtered and the filtrate was concentrated in vacuo. The residue was purified by column chromatography on silica gel (PE/EtOAc=1/1, DCM/MeOH=10/1) to give 2-(1,1-difluoroethyl)-4-((1s,3s)-3-methoxycyclobutoxy)pyridine 1-oxide (3.5 g, 88.8% yield) as a colorless oil. 1H NMR: (400 MHz, CDCl₃) δ ppm 8.19 (d, J=7.2 Hz, 1H), 7.08 (d, J=7.2 Hz, 1H), 6.80 (dd, J=3.4, 7.2 Hz, 1H), 4.41-4.34 (m, 1H), 3.73-3.70 (m, 1H), 3.30 (s, 3H), 2.97-2.90 (m, 2H), 2.25 (t, J=22.4 Hz, 3H), 2.20-2.16 (m, 2H).

### Preparation 479: 2-(1,1-difluoroethyl)-4-((1r,3r)-3-methoxycyclobutoxy)pyridine 1-oxide

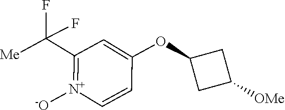

2-(1,1-Difluoroethyl)-4-((1r,3r)-3-methoxycyclobutoxy)pyridine 1-oxide was obtained as a colorless oil, 7 g, 93.8%, from 2-(1,1-difluoroethyl)-4-((1r,3r)-3-methoxycyclobutoxy)pyridine (Preparation 477), following the procedure described in Preparation 478. 1H NMR: (400 MHz, CDCl₃) δ ppm 8.14 (d, J=7.2 Hz, 1H), 7.09 (d, J=7.2 Hz, 1H), 6.65 (dd, J=2.4, 7.2 Hz, 1H), 4.94-4.79 (m, 1H), 4.16-4.08 (m, 1H), 3.28 (s, 3H), 2.53-2.38 (m, 4H), 2.25 (t, J=19.2 Hz, 3H).

### Preparation 480: 2-chloro-6-(1,1-difluoroethyl)-4-((1s,3s)-3-methoxycyclobutoxy)pyridine

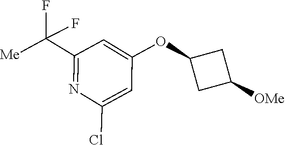 [SMILES: CO[C@H]1C[C@@H](Oc2cc(Cl)nc(C(C)(F)F)c2)C1]

To a solution of 2-(1,1-difluoroethyl)-4-((1s,3s)-3-methoxycyclobutoxy)pyridine 1-oxide (Preparation 478, 5.00 g, 19.29 mmol) was added POCl₃ (40 mL, 429 mmol) and the reaction mixture was stirred at 100° C. for 16 h. The mixture was concentrated, dissolved in DCM (20 mL), the solution was poured into water (50 mL) and quenched with sat.NaHCO₃ (40 mL) until pH>8. The aqueous layer was extracted with DCM (60 mL×3). The combined organic fractions were washed with brine (100 mL), dried over Na₂SO₄ and concentrated. The crude product was purified by column chromatography on silica gel (PE/EtOAc=10/1) to give 2-chloro-6-(1,1-difluoroethyl)-4-((1s,3s)-3-methoxycyclobutoxy)pyridine (2.74 g, 51.2% yield) as a yellow oil. LCMS m/z=278.1 [M+H]⁺

### Preparation 481: 2-chloro-6-(1,1-difluoroethyl)-4-((1r,3r)-3-methoxycyclobutoxy)pyridine

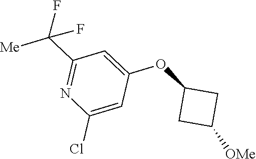 [SMILES: CO[C@H]1C[C@H](Oc2cc(Cl)nc(C(C)(F)F)c2)C1]

2-Chloro-6-(1,1-difluoroethyl)-4-((1r,3r)-3-methoxycyclobutoxy)pyridine was obtained as a yellow oil, 3.8 g, 50.7% from 2-(1,1-difluoroethyl)-4-((1r,3r)-3-methoxycyclobutoxy)pyridine 1-oxide (Preparation 479), following the procedure described in Preparation 480. LCMS m/z=278.1 [M+H]⁺

### C-3 Substituents

### Preparation 482: benzyl-N-(1-methylcyclopropyl)pyrrolidin-3-amine

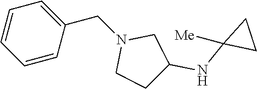 [SMILES: CC1(NC2CCN(Cc3ccccc3)C2)CC1]

To a solution of 1-benzylpyrrolidin-3-one (2 g, 11.41 mmol) in MeOH (25 mL) was added 1-methylcyclopropan-1-amine hydrochloride (1.97 g, 13.7 mmol) and the mixture was stirred for 1 h at 25° C. NaBH₃CN (1.08 g, 17.12 mmol) was added and the reaction stirred for another 16 h at 25° C. The reaction mixture was concentrated and extracted with DCM (100 mL×3). The combined organic layers were washed with brine, dried over anhydrous Na₂SO₄ and concentrated under reduced pressure. The residue was purified by silica gel chromatography (0/100 to 100/0 EtOAc/PE) to give 1-benzyl-N-(1-methylcyclopropyl)pyrrolidin-3-amine (1 g, 36.1% yield) as yellow oil. LCMS m/z=231.2 [M+H]⁺

### Preparation 483: tert-butyl (1-benzylpyrrolidin-3-yl)(1-methylcyclopropyl)carbamate

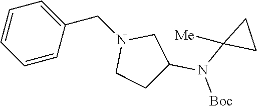 [SMILES: CC(C)(C)OC(=O)N(C1CCN(Cc2ccccc2)C1)C1(C)CC1]

To a solution of 1-benzyl-N-(1-methylcyclopropyl)pyrrolidin-3-amine (Preparation 482, 1 g, 4.34 mmol) in DCM (20 mL) was added (Boc)₂O (1.42 g, 6.51 mmol) and TEA (1.32 g, 13.02 mmol) and the reaction mixture was stirred for 16 h. The mixture was diluted with water (100 mL) and extracted with DCM (100 mL×3). The combined organic layers were washed with brine (50 mL×2), dried over anhydrous Na₂SO₄, filtered and concentrated under reduced pressure to give a residue, which was purified by prep-HPLC-F to give tert-butyl (1-benzylpyrrolidin-3-yl)(1-methylcyclopropyl)carbamate (500 mg, 34.9% yield) as yellow oil. LCMS m/z=331.2 [M+H]⁺

### Preparation 484: tert-butyl (1-methylcyclopropyl)(pyrrolidin-3-yl)carbamate

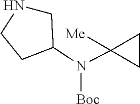 [SMILES: CC(C)(C)OC(=O)N(C1CCNC1)C1(C)CC1]

To a solution of tert-butyl (1-benzylpyrrolidin-3-yl)(1-methylcyclopropyl)carbamate (Preparation 483, 450 mg, 1.36 mmol) in MeOH (5 mL) was added Pd(OH)₂/C (95.62 mg, 0.136 mmol, 20% purity) and the reaction was stirred at 45° C. for 24 h under H₂ (45 Psi). The mixture was filtered and the filtrate was concentrated to give tert-butyl (1-methylcyclopropyl)(pyrrolidin-3-yl)carbamate (300 mg, 88.0% yield) as yellow oil. 1H NMR: (400 MHz, DMSO-d₆) δ ppm 3.90-3.83 (m, 1H), 3.64-2.99 (m, 3H), 2.93-2.89 (m, 1H), 2.83-2.49 (m, 1H), 2.02-1.98 (m, 1H), 1.91-1.85 (m, 1H), 1.41 (s, 9H), 1.21 (s, 3H), 0.81-0.72 (m, 2H), 0.66-0.62 (m, 2H).

### Preparation 485: tert-butyl 3-(bis(methyl-d₃)amino)pyrrolidine-1-carboxylate

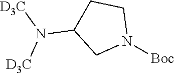 [SMILES: [2H]C([2H])([2H])N(C1CCN(C(=O)OC(C)(C)C)C1)C([2H])([2H])[2H]]

DIPEA (376 μL, 2.16 mmol) was added to a solution of tert-butyl 3-oxopyrrolidine-1-carboxylate (200 mg, 1.08 mmol) and bis(methyl-d₃)amine hydrochloride (104 mg, 1.19 mmol) in DCM (6 mL) and the solution stirred for 15 min. AcOH (124 μL, 2.16 mmol), followed by NaBH(OAc)₃ (343 mg, 1.62 mmol) were added and the reaction mixture stirred overnight. The reaction was quenched by slow addition of NaHCO₃ solution, extracted with EtOAc (3×), washed with brine, dried over Na₂SO₄, filtered and evaporated to dryness. The crude product was purified by SCX column, eluting with MeOH then 2M NH₃ in MeOH, to give tert-butyl 3-(bis(methyl-d₃)amino)pyrrolidine-1-carboxylate (202 mg, 85% yield) as dark colored liquid. ¹H NMR (400 MHz, MeOD-d₄) δ ppm 3.56-3.64 (m, 1H), 3.46-3.55 (m, 1H), 3.20-3.29 (m, 1H), 3.06 (dd, J=10.51, 8.51 Hz, 1H), 2.72-2.84 (m, 1H), 2.08-2.18 (m, 1H), 1.68-1.83 (m, 1H), 1.46 (s, 9H).

### Preparation 486: tert-butyl 3-(bis(methyl-d3)amino)-3-methylpyrrolidine-1-carboxylate

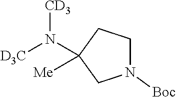 [SMILES: [2H]C([2H])([2H])N(C([2H])([2H])[2H])C1(C)CCN(C(=O)OC(C)(C)C)C1]

To a solution of tert-butyl 3-amino-3-methylpyrrolidine-1-carboxylate (202 mg, 1.01 mmol) in THF (5 mL) was added 60% NaH (124 mg, 3.10 mmol). After 15 min, iodomethane-d₃ (138 μL, 2.22 mmol) was added and the reaction was stirred at rt for 2.5 h. The reaction was quenched by careful addition of saturated NaHCO₃ solution. The mixture was extracted with EtOAc, washed with saturated brine, dried over Na₂SO₄, filtered and evaporated. The crude was purified by SCX (5 g, eluting with MeOH then 2M NH₃/MeOH) to give tert-butyl 3-(bis(methyl-d₃)amino)-3-methylpyrrolidine-1-carboxylate (186 mg, 79% yield). ¹H NMR (400 MHz, MeOD-d₄) δ ppm 3.46-3.54 (m, 1H), 3.34-3.45 (m, 1H), 3.14-3.22 (m, 1H), 1.80-1.94 (m, 2H), 1.46 (s, 9H), 1.22-1.31 (m, 1H), 1.08 (s, 3H).

### Preparation 487: tert-butyl 3-(methoxy-d3)-3-methylpyrrolidine-1-carboxylate

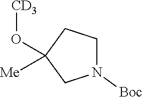 [SMILES: [2H]C([2H])([2H])OC1(C)CCN(C(=O)OC(C)(C)C)C1]

tert-Butyl 3-(methoxy-d3)-3-methylpyrrolidine-1-carboxylate was obtained from tert-butyl 3-hydroxy-3-methylpyrrolidine-1-carboxylate and iodomethane-d3, following a similar procedure to that described in Preparation 486. ¹H NMR (400 MHz, MeOD-d₄) δ ppm 3.45-3.52 (m, 1H), 3.34-3.42 (m, 2H), 3.10 (t, J=12.0 Hz, 1H), 2.09 (m, 1H), 1.77 (m, 1H), 1.46 (s, 9H), 1.32 (d, J=4.5 Hz, 3H).

### Preparation 488: N,N-bis(methyl-d3)pyrrolidin-3-amine hydrochloride

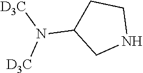 [SMILES: [2H]C([2H])([2H])N(C1CCNC1)C([2H])([2H])[2H]]

HCl/dioxane (4 M, 2.3 mL) was added to a solution of tert-butyl 3-(bis(methyl-d₃)amino)pyrrolidine-1-carboxylate (Preparation 485, 202 mg, 0.91 mmol) in DCM (5 mL) and the reaction was stirred at rt overnight. The reaction mixture was evaporated to dryness to give N,N-bis(methyl-d₃)pyrrolidin-3-amine hydrochloride (220 mg, crude). ¹H NMR (400 MHz, MeOD-d₄) δ ppm 4.14 (quin, J=8.01 Hz, 1H), 3.81 (dd, J=12.76, 8.26 Hz, 1H), 3.57-3.66 (m, 2H), 3.40 (ddd, J=11.76, 9.51, 7.26 Hz, 1H), 2.58 (dtd, J=13.63, 7.69, 7.69, 4.00 Hz, 1H), 2.28-2.40 (m, 1H).

### Preparation 489: 3-methyl-N,N-bis(methyl-d₃)pyrrolidin-3-amine hydrochoride

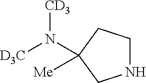 [SMILES: [2H]C([2H])([2H])N(C([2H])([2H])[2H])C1(C)CCNC1]

3-Methyl-N,N-bis(methyl-d3)pyrrolidin-3-amine hydrochoride was obtained as an off-white foam, from tert-butyl 3-(bis(methyl-d₃)amino)-3-methylpyrrolidine-1-carboxylate (Preparation 486) following the procedure described in Preparation 488. ¹H NMR (400 MHz, MeOD-d₄) δ ppm 3.60-3.76 (m, 2H), 3.43-3.60 (m, 2H), 2.39-2.58 (m, 1H), 2.23-2.39 (m, 1H), 1.59 (s, 3H).

### Preparation 490: 3-(methoxy-d₃)-3-methylpyrrolidine hydrochloride

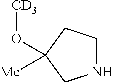 [SMILES: [2H]C([2H])([2H])OC1(C)CCNC1]

3-(Methoxy-d₃)-3-methylpyrrolidine hydrochloride was obtained, from tert-butyl 3-(methoxy-d3)-3-methylpyrrolidine-1-carboxylate (Preparation 487), following the procedure described in Preparation 488. ¹H NMR (400 MHz, MeOD-d₄) δ ppm 3.56-3.78 (m, 1H), 3.34-3.44 (m, 2H), 2.99 (d, J=12.01 Hz, 1H), 2.25-2.35 (m, 1H), 1.86 (dt, J=13.88, 10.07 Hz, 1H), 1.41 (s, 3H), 0.82-0.93 (m, 2H)

### Preparation 491: tert-butyl 3-((4-methoxybenzyl)(methyl)amino)-4-methylpyrrolidine-1-carboxylate

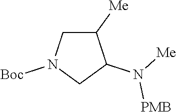

To a solution of tert-butyl 3-methyl-4-oxopyrrolidine-1-carboxylate (5.0 g, 25.1 mmol) in MeOH (130 mL) was added 1-(4-methoxyphenyl)-N-methylmethanamine (4.6 g, 30.11 mmol) and NaBH₃CN (3.2 g, 50.2 mmol) and the reaction was stirred at 20° C. for 2 h. The mixture was concentrated and then water (50 mL) was added. The mixture was extracted with EtOAc (50 mL×2), the combined organic layers were washed with brine (50 mL×2), dried over Na₂SO₄, filtered and concentrated under vacuum. The crude was purified by column chromatography on silica gel (from PE/EtOAc=1/0 to 1/1) to yield tert-butyl 3-((4-methoxybenzyl)(methyl)amino)-4-methylpyrrolidine-1-carboxylate (3.2 g, 38.4% yield) as a yellow oil. ¹H NMR (400 MHz, CDCl₃) δ ppm: 7.25-7.22 (m, 2H), 6.87-6.85 (m, 2H), 3.80 (s, 3H), 3.58-3.54 (m, 2H), 3.42-3.40 (m, 1H), 3.23-3.19 (m, 2H), 2.76-2.70 (m, 1H), 2.40-2.38 (m, 1H), 2.13-2.12 (m, 1H), 2.04-2.03 (m, 1H), 2.02 (s, 3H) 1.46 (s, 9H), 1.06-1.04 (m, 3H).

### Preparation 492: N-(4-methoxybenzyl)-N,4-dimethylpyrrolidin-3-amine hydrochloride

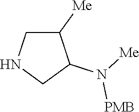

To a solution of tert-butyl 3-((4-methoxybenzyl)(methyl)amino)-4-methylpyrrolidine-1-carboxylate (Preparation 491, 3.2 g, 9.63 mmol) in DCM (20 mL) was added HCl/dioxane (4 M, 30 mL) and the reaction was stirred at 25° C. for 1 h. The mixture was concentrated under vacuum to give N-(4-methoxybenzyl)-N,4-dimethylpyrrolidin-3-amine hydrochloride (3.5 g, crude) as a white solid. LCMS m/z=235.1 [M+H]⁺

### Preparation 493: tert-butyl 3-(vinyloxy)pyrrolidine-1-carboxylate

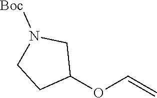

To a solution of tert-butyl 3-hydroxypyrrolidine-1-carboxylate (2.0 g, 10.68 mmol) in 1-(vinyloxy)butane (10 mL) was added 4,7-diphenyl-1,10-phenanthroline (177.5 mg, 0.534 mmol), Pd(OAc)₂ (119.9 mg, 0.534 mmol) and TEA (540.4 mg, 5.34 mmol) and the reaction was stirred at 80° C. for 2 h under N₂ in microwave. The reaction was concentrated under reduced pressure and the residue was purified by chromatography on silica gel (PE/EtOAc=20/1 to 3/1) to give tert-butyl 3-(vinyloxy)pyrrolidine-1-carboxylate (1.4 g, 61.5% yield) as colorless oil. ¹H NMR: (500 MHz, CDCl₃) δ ppm: 6.35-6.31 (m, 1H), 5.40 (br s, 1H), 4.21 (d, J=13.0 Hz, 1H), 4.08 (d, J=7.0 Hz, 1H), 3.54-3.39 (m, 4H), 2.11-2.07 (m, 1H), 2.00-1.98 (m, 1H), 1.46 (s, 9H).

### Preparation 494: tert-butyl 3-cyclopropoxypyrrolidine-1-carboxylate

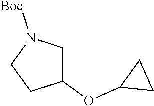 [SMILES: CC(C)(C)OC(=O)N1CCC(OC2CC2)C1]

To a solution of tert-butyl 3-(vinyloxy)pyrrolidine-1-carboxylate (Preparation 493, 1.4 g, 6.56 mmol) and chloroethane (3.5 g, 19.69 mmol) in DCE (20 mL) was added slowly, ZnEt₂ (1 M, 10.5 mL) at 0° C. under N₂. The resulting mixture was stirred at 0° C. for 2 h. The mixture was treated with H₂O (50 mL) and extracted with EtOAc (30 mL×3). The combined organic phase was washed with brine (30 mL×2), dried over Na₂SO₄ and filtered. The filtrate was concentrated under reduced pressure and the residue was purified by chromatography on silica gel (PE/EtOAc=20/1 to 3/1) to give tert-butyl 3-cyclopropoxypyrrolidine-1-carboxylate (1.1 g, 73.7% yield) as colorless oil. 1H NMR (400 MHz, CDCl₃) δ ppm: 4.15-4.13 (m, 1H), 3.52-3.40 (m, 4H), 3.29-3.27 (m, 1H), 2.04-1.94 (m, 2H), 1.45 (s, 9H), 0.61-0.49 (m, 4H).

### Preparation 495: tert-butyl 3-(diethylamino)-4-methylpyrrolidine-1-carboxylate

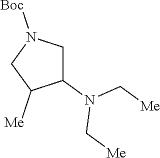

To a solution of tert-butyl 3-methyl-4-oxopyrrolidine-1-carboxylate (500 mg, 2.51 mmol) in MeOH (10 mL) was added Et₂NH (0.31 mL, 3.01 mmol), NaBH₃CN (315.4 mg, 5.02 mmol) and the mixture was stirred at 25° C. for 2 h. The mixture was concentrated under reduced pressure to give the residue, which was purified by column chromatography (PE/EtOAc=15/1 to 0/1) on silica gel to give tert-butyl 3-(diethylamino)-4-methylpyrrolidine-1-carboxylate (170 mg, 0.66 mmol) as colorless oil. 1H NMR: (400 MHz, CDCl₃) δ ppm: 3.62-3.41 (m, 1H), 3.39-3.33 (m 1H), 3.30-3.20 (m, 1H), 3.18-3.07 (m, 2H), 3.07-2.94 (m, 1H), 2.68-2.50 (m, 4H), 2.34-2.19 (m, 1H), 1.45 (s, 9H), 1.06-0.89 (m, 9H)

### Preparation 496: tert-butyl 4′-methyl-[1,3′-bipyrrolidine]-1′-carboxylate

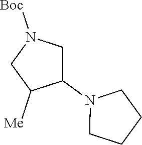 [SMILES: CC1CN(C(=O)OC(C)(C)C)CC1N1CCCC1]

tert-Butyl 4′-methyl-[1,3′-bipyrrolidine]-1′-carboxylate was obtained as a colorless oil, 488.2 mg, 38.2% from tert-butyl 3-methyl-4-oxopyrrolidine-1-carboxylate and pyrrolidine, following a similar procedure to that described in Preparation 495. LCMS m/z=255.3 [M+H]⁺

### Preparation 497: 3-cyclopropoxypyrrolidine hydrochloride

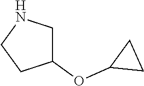

To a solution of tert-butyl 3-cyclopropoxypyrrolidine-1-carboxylate (Preparation 494, 1.1 g, 4.84 mmol) in DCM (8 mL) was added HCl/dioxane (4 M, 8.0 mL) and the reaction was stirred at 20° C. for 3 h. The mixture was concentrated under reduced pressure to give 3-cyclopropoxypyrrolidine hydrochloride (780.6 mg, crude) as yellow oil.

### Preparation 498: N,N-diethyl-4-methylpyrrolidin-3-amine hydrochloride

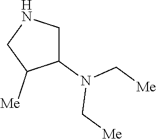 [SMILES: CCN(CC)C1CNCC1C]

N,N-Diethyl-4-methylpyrrolidin-3-amine hydrochloride was obtained as a white solid, from tert-butyl 3-(diethylamino)-4-methylpyrrolidine-1-carboxylate (Preparation 495) following the procedure described in Preparation 497. ¹H NMR (500 MHz, DMSO-d₆) δ ppm: 9.77 (s, 1H), 3.91-4.03 (m, 1H), 3.56-3.60 (m, 1H), 3.32-3.40 (m, 1H), 2.97-3.30 (m, 6H), 2.66-2.74 (m, 1H), 1.06-1.31 (m, 9H)

### Preparation 499: 4′-methyl-1,3′-bipyrrolidine hydrochloride

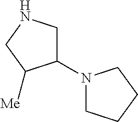 [SMILES: CC1CNCC1N1CCCC1]

4′-Methyl-1,3′-bipyrrolidine hydrochloride was obtained as brown oil, 340 mg, crude, from tert-butyl 4′-methyl-[1,3′-bipyrrolidine]-1′-carboxylate (Preparation 496) following the procedure described in Preparation 497. LCMS m/z=155.2 [M+H]⁺

### From JH2 Prov File

### Preparation 500: N-(3-bromo-1-(6-(2-fluoropropan-2-yl)pyrazin-2-yl)-1H-pyrazolo[4,3-c]pyridin-6-yl)acetamide

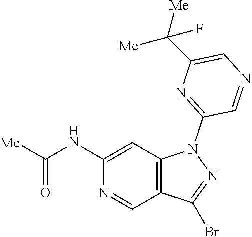 [SMILES: CC(=O)Nc1cc2c(cn1)c(Br)nn2-c1cncc(C(C)(C)F)n1]

To a solution of N-(3-bromo-1H-pyrazolo[4,3-c]pyridin-6-yl)acetamide (Preparation 366, 300 mg, 1.18 mmol) in DMF (5 mL) was added 2-chloro-6-(2-fluoropropan-2-yl)pyrazine (225.9 mg, 1.29 mmol) and Cs₂CO₃ (766.4 mg, 2.35 mmol) and the mixture was stirred at 100° C. for 2 h. The mixture was diluted with water (20 mL) and extracted with EtOAc (25 mL×3). The combined organic layers were washed with brine (25 mL), dried over Na₂SO₄, filtered and concentrated to give a residue which was purified on silica gel column chromatography (PE/EtOAc=1/0 to 1/1) to give N-(3-bromo-1-(6-(2-fluoropropan-2-yl)pyrazin-2-yl)-1H-pyrazolo[4,3-c]pyridin-6-yl)acetamide (450 mg, 97.3% yield) as a white solid. ¹H NMR: (400 MHz, CDCl₃) δ ppm 9.43 (s, 1H), 9.28 (s, 1H), 8.77 (s, 1H), 8.69 (s, 1H), 2.30 (s, 3H), 1.96 (s, 3H), 1.90 (s, 3H).

### Preparations 501 to 503

The compounds in the following table were prepared from N-(3-bromo-1H-pyrazolo[4,3-c]pyridin-6-yl)acetamide (Preparation 366) and the corresponding halo pyrimidine, following a similar procedure to that described in Preparation 500.

| Preparation | |
|---|---|
| No. | Name, Structure, Starting material (SM), Data |
| 501 | N-(3-bromo-1-(2-(2-fluoropropan-2-yl)-6-methylpyrimidin- |
| | 4-yl)-1H-pyrazolo[4,3-c]pyridin-6-yl)acetamide |
| | 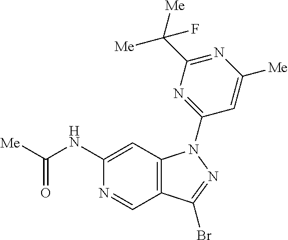 [SMILES: CC(=O)Nc1cc2c(cn1)c(Br)nn2-c1cc(C)nc(C(C)(C)F)n1] |
| | yellow solid, 180 mg, 75.2% |
| | 4-chloro-2-(2-fluoropropan-2-yl)-6-methylpyrimidine |
| | (Preparation 411), ¹H NMR (400 MHz, CDCl₃) |
| | δ ppm: 9.53 (s, 1H), 8.67 (s, 1H), 8.25 (s, 1H), 7.72 |
| | (s, 1H), 2.67 (s, 3H), 2.30 (s, 3H), 1.99 (s, 3H), 1.94 (s, 3H). |
| 502 | N-(3-bromo-1-(6-ethyl-2-(2-fluoropropan-2-yl)pyrimidin- |
| | 4-yl)-1H-pyrazolo[4,3-c]pyridin-6-yl)acetamide |
| | 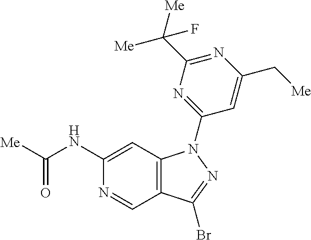 [SMILES: CCc1cc(-n2nc(Br)c3cnc(NC(C)=O)cc32)nc(C(C)(C)F)n1] |
| | SM: 4-chloro-6-ethyl-2-(2-fluoropropan-2-yl)pyrimidine |
| | (Preparation 412) yellow solid, 286.5 mg, 86.7% yield, |
| | ¹H NMR (500 MHz, CDCl₃) δ ppm: 9.53 |
| | (s, 1H), 8.67 (s, 1H), 8.25 (s, 1H), 7.71 (s, 1H), 2.94 (q, J = |
| | 7.5 Hz, 2H), 2.29 (s, 3H), 1.99 (s, 3H), 1.94 (s, 3H), 1.38 (t, |
| | J = 8.0 Hz, 3H) |
| 503 | N-(3-bromo-1-(6-cyclopropyl-2-(1,1-difluoroethyl)pyrimidin- |
| | 4-yl)-1H-pyrazolo[4,3-c]pyridin-6-yl)acetamide |
| | 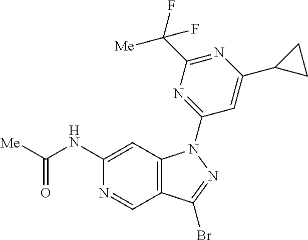 [SMILES: CC(=O)Nc1cc2c(cn1)c(Br)nn2-c1cc(C2CC2)nc(C(C)(F)F)n1] |
| | SM: 4-chloro-6-cyclopropyl-2-(1,1-difluoroethyl)pyrimidine |
| | (Preparation 415) white solid, 393 mg, 63% |
| | ¹H NMR (500 MHz, CDCl₃) δ ppm: 9.55 (s, 1H), 8.66 |
| | (s, 1H), 8.19 (s, 1H), 7.77 (s, 1H), 2.29 (s, 3H), 2.21 (t, |
| | J = 18.5 Hz, 3H), 2.17-2.16 (m, 1H), 1.30-1.27 (m, |
| | 2H), 1.19-1.17 (m, 2H). |

### Preparation 504: N-(3-bromo-1-(6-(1,1-difluoroethyl)pyrazin-2-yl)-1H-pyrazolo[4,3-c]pyridin-6-yl)acetamide

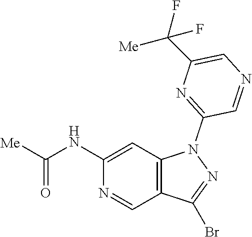 [SMILES: CC(=O)Nc1cc2c(cn1)c(Br)nn2-c1cncc(C(C)(F)F)n1]

To a solution of N-(3-bromo-1H-pyrazolo[4,3-c]pyridin-6-yl)acetamide (Preparation 366, 1 g, 3.92 mmol) in DMF (10 mL) was added 2-chloro-6-(1,1-difluoroethyl)pyrazine (1.1 g, 5.88 mmol) and Cs₂CO₃ (1.3 g, 3.92 mmol) and the reaction mixture was stirred at 100° C. for 16 h. The mixture was cooled to rt, filtered, the filter cake was washed with PE (20 mL×2) and dried to give N-(3-bromo-1-(6-(1,1-difluoroethyl)pyrazin-2-yl)-1H-pyrazolo[4,3-c]pyridin-6-yl)acetamide (1.5 g, 96.3% yield) as a gray solid. ¹H NMR (500 MHz, DMSO-d₆) δ: ppm 10.95 (s, 1H), 9.34 (d, J=7.0 Hz, 2H), 8.92 (s, 1H), 8.85 (s, 1H), 2.28 (t, J=19.5 Hz, 3H), 2.17 (s, 3H).

### Preparation 505: N-(3-bromo-1-(2-(1,1-difluoroethyl)-6-methoxypyrimidin-4-yl)-1H-pyrazolo[4,3-c]pyridin-6-yl)acetamide

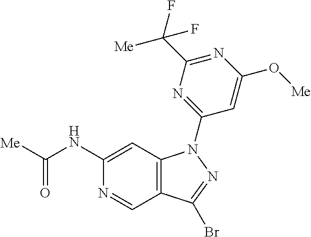 [SMILES: COc1cc(-n2nc(Br)c3cnc(NC(C)=O)cc32)nc(C(C)(F)F)n1]

N-(3-Bromo-1-(2-(1,1-difluoroethyl)-6-methoxypyrimidin-4-yl)-1H-pyrazolo[4,3-c]pyridin-6-yl)acetamide was obtained as a brown solid, 171 mg, 68% from N-(3-bromo-1H-pyrazolo[4,3-c]pyridin-6-yl)acetamide (Preparation 366) and 4-chloro-2-(2-fluoropropan-2-yl)-6-methoxypyrimidine (Preparation 208) following the procedure described in Preparation 504. LCMS m/z=427.0 [M+H]⁺

### Preparation 506: N-(1-(4-(1,1-difluoroethyl)pyrimidin-2-yl)-3-iodo-1H-pyrazolo[4,3-c]pyridin-6-yl)acetamide

30-3 int 3

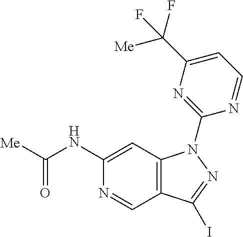 [SMILES: CC(=O)Nc1cc2c(cn1)c(I)nn2-c1nccc(C(C)(F)F)n1]

N-(1-(4-(1,1-Difluoroethyl)pyrimidin-2-yl)-3-iodo-1H-pyrazolo[4,3-c]pyridin-6-yl)acetamide was obtained as a yellow solid, 8.2 g, 74.9%, from 2-chloro-4-(1,1-difluoroethyl)pyrimidine and N-(3-iodo-1H-pyrazolo[4,3-c]pyridin-6-yl)acetamide (Preparation 3) following a similar procedure to that described in Preparation 504. LCMS m/z=444.9 [M+H]⁺

### Preparation 507: N-(3-bromo-1-(6-(1,1-difluoroethyl)-4-methylpyridin-2-yl)-1H-pyrazolo[4,3-c]pyridin-6-yl)acetamide

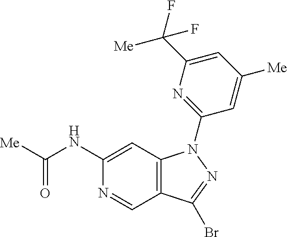 [SMILES: CC(=O)Nc1cc2c(cn1)c(Br)nn2-c1cc(C)cc(C(C)(F)F)n1]

To a solution of N-(3-bromo-1H-pyrazolo[4,3-c]pyridin-6-yl)acetamide (Preparation 366, 150 mg, 0.59 mmol) in dioxane (3 mL) was added 2-bromo-6-(1,1-difluoroethyl)-4-methylpyridine (Preparation 416, 166.6 mg, 0.71 mmol), Pd₂(dba)₃ (53.9 mg, 58.8 μmol), Xantphos (68.1 mg, 0.118 mmol) and Cs₂CO₃ (383.2 mg, 1.18 mmol) and the resulting mixture was stirred at 100° C. for 3 h under N₂. The mixture was concentrated, the residue diluted with H₂O (30 mL) and extracted with EtOAc (30 mL×3). The combined organic layers were washed with brine (30 mL×2), dried over Na₂SO₄, filtered and concentrated to give a residue which was purified on silica gel column chromatography (PE/EtOAc=20/1 to 1/1) to give N-(3-bromo-1-(6-(1,1-difluoroethyl)-4-methylpyridin-2-yl)-1H-pyrazolo[4,3-c]pyridin-6-yl)acetamide (146.5 mg, 60.7% yield) as a white solid. ¹H NMR (500 MHz, CDCl₃) δ ppm: 9.45 (s, 1H), 8.66 (s, 1H), 8.22 (s, 1H), 7.90 (s, 1H), 7.43 (s, 1H), 2.52 (s, 3H), 2.33-2.25 (m, 6H).

### Preparation 508 to 510

The compounds in the following table were prepared from N-(3-bromo-1H-pyrazolo[4,3-c]pyridin-6-yl)acetamide (Preparation 366) and the appropriate aryl halide, following a similar procedure to that described in Preparation 507.

| Preparation | |
|---|---|
| No | Name, Structure, Starting Halide, Data |
| 508 | N-(3-bromo-1-(2-(1,1-difluoroethyl)-6-isopropylpyrimidin- |
| | 4-yl)-1H-pyrazolo[4,3-c]pyridin-6-yl)acetamide |
| | 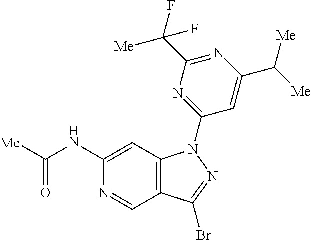 [SMILES: CC(=O)Nc1cc2c(cn1)c(Br)nn2-c1cc(C(C)C)nc(C(C)(F)F)n1] |
| | 4-chloro-2-(1,1-difluoroethyl)-6-isopropylpyrimidine |
| | (Preparation 451) a white solid, 470 mg, 65.5%, LCMS |
| | m/z = 439.0 [M + H]⁺ |
| 509 | N-(3-bromo-1-(6-(1,1-difluoroethyl)-4-methoxypyridin- |
| | 2-yl)-1H-pyrazolo[4,3-c]pyridin-6-yl)acetamide |
| | 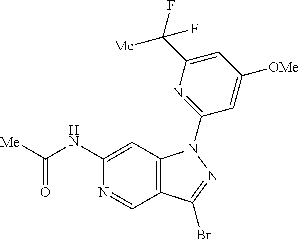 [SMILES: COc1cc(-n2nc(Br)c3cnc(NC(C)=O)cc32)nc(C(C)(F)F)c1] |
| | 2-chloro-6-(1,1-difluoroethyl)-4-methoxypyridine |
| | (Preparation 133) white solid, 100 mg, 46.5% yield. |
| | LCMS m/z = 426.1 [M + H]⁺ |
| 510 | N-(3-bromo-1-(6-(1,1-difluoroethyl)pyridin-2-yl)- |
| | 1H-pyrazolo[4,3-c]pyridin-6-yl)acetamide |
| | 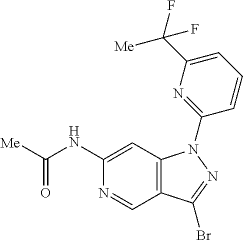 [SMILES: CC(=O)Nc1cc2c(cn1)c(Br)nn2-c1cccc(C(C)(F)F)n1] |
| | 2-bromo-6-(1,1-difluoroethyl)pyridine |
| | a yellow solid. LCMS m/z = 97.7 [M + H]⁺ |

### Preparation 511: N-(3-bromo-1-(6-(2-fluoropropan-2-yl)pyridin-2-yl)-1H-pyrazolo[4,3-c]pyridin-6-yl)acetamide

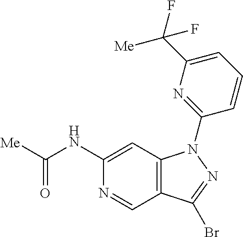 [SMILES: CC(=O)Nc1cc2c(cn1)c(Br)nn2-c1cccc(C(C)(F)F)n1]

To a solution of 2-bromo-6-(2-fluoropropan-2-yl)pyridine (Preparation 132, 400 mg, 1.83 mmol) in DMSO (10 mL) was added N-(3-bromo-1H-pyrazolo[4,3-c]pyridin-6-yl)acetamide (Preparation 366, 467.9 mg, 1.83 mmol), K₃PO₄ (778.7 mg, 3.67 mmol), 2,6-DFPAO (147.6 mg, 0.73 mmol) and CuI (69.9 mg, 0.37 mmol) and the reaction was stirred at 130° C. for 16 h under N₂. The mixture was quenched with H₂O (20 mL) and extracted with EtOAc (20 mL×3). The combined organic phase was washed with brine (20 mL×3), dried over Na₂SO₄ and filtered. The filtrate was concentrated under reduced pressure and the residue purified by chromatography on silica gel (PE/EtOAc=1/1) to give N-(3-bromo-1-(6-(2-fluoropropan-2-yl)pyridin-2-yl)-1H-pyrazolo[4,3-c]pyridin-6-yl)acetamide (251.8 mg, 35% yield) as a white solid. ¹H NMR (400 MHz, CDCl₃) δ: ppm 9.45 (s, 1H), 8.66 (d, J=0.8 Hz, 1H), 8.36-8.34 (m, 1H), 7.91-7.86 (m, 2H), 7.51-7.47 (m, 1H), 2.29 (s, 3H), 1.94 (s, 3H), 1.88 (s, 3H).

### Preparation 512: tert-butyl 3-(6-acetamido-1-(6-(1,1-difluoroethyl)pyridin-2-yl)-1H-pyrazolo[4,3-c]pyridin-3-yl)-3,6-diazabicyclo[3.1.1]heptane-6-carboxylate

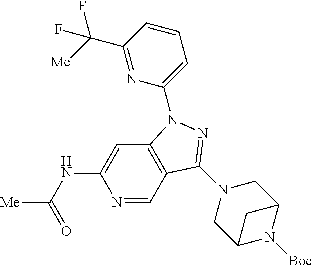 [SMILES: CC(=O)Nc1cc2c(cn1)c(N1CC3CC(C1)N3C(=O)OC(C)(C)C)nn2-c1cccc(C(C)(F)F)n1]

To a solution of N-(3-bromo-1-(6-(1,1-difluoroethyl)pyridin-2-yl)-1H-pyrazolo[4,3-c]pyridin-6-yl)acetamide (Preparation 510, 200 mg, 0.51 mmol) in dioxane (2 mL) was added tert-butyl 3,6-diazabicyclo[3.1.1]heptane-6-carboxylate (500.4 mg, 2.52 mmol), Pd₂(dba)₃ (46.23 mg, 0.05 mmol), Xantphos (29.21 mg, 0.05 mmol) and Cs₂CO₃ (329.0 mg, 1.01 mmol) and the reaction mixture was heated at 100° C. for 16 h. The mixture was diluted with water (50 mL) and extracted with EtOAc (100 mL×2). The combined organic phase was washed with brine (80 mL), dried with anhydrous Na₂SO₄, filtered and concentrated in vacuo. The residue was purified by silica gel chromatography (0/100 to 100/0 EtOAc/PE) to afford tert-butyl 3-(6-acetamido-1-(6-(1,1-difluoroethyl)pyridin-2-yl)-1H-pyrazolo[4,3-c]pyridin-3-yl)-3,6-diazabicyclo[3.1.1]heptane-6-carboxylate (200 mg, 75.6% yield) as yellow oil. LCMS m/z=514.3 [M+H]⁺

### Preparation 513: 3-(6-chloro-1-(tetrahydro-2H-pyran-2-yl)-1H-pyrazolo[4,3-c]pyridin-3-yl)-6-oxa-3-azabicyclo[3.1.1]heptane

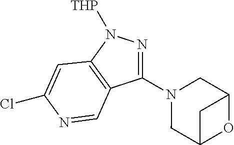

To a solution of 3-bromo-6-chloro-1-(tetrahydro-2H-pyran-2-yl)-1H-pyrazolo[4,3-c]pyridine (300 mg, 0.948 mmol) and 6-oxa-3-azabicyclo[3.1.1]heptane (154.2 mg, 1.14 mmol) in dioxane (10 mL) was added Pd₂(dba)₃ (86.8 mg, 0.095 mmol), Xantphos (109.7 mg, 0.19 mmol) and Cs₂CO₃ (926.3 mg, 2.84 mmol) and the reaction mixture was stirred at 100° C. for 4 h. The mixture was concentrated under vacuum and the crude product was purified by column chromatography (EtOAc/PE 0/100 to 15/85) to give 3-(6-chloro-1-(tetrahydro-2H-pyran-2-yl)-1H-pyrazolo[4,3-c]pyridin-3-yl)-6-oxa-3-azabicyclo[3.1.1]heptane (220 mg, 69.3% yield) as yellow oil. LCMS m/z=335.1 [M+H]⁺

### Preparation 514: 5-(6-chloro-1-(tetrahydro-2H-pyran-2-yl)-1H-pyrazolo[4,3-c]pyridin-3-yl)-2-oxa-5-azabicyclo[2.2.1]heptane

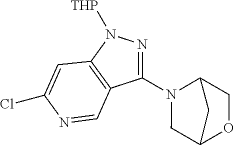

5-(6-Chloro-1-(tetrahydro-2H-pyran-2-yl)-1H-pyrazolo[4,3-c]pyridin-3-yl)-2-oxa-5-azabicyclo[2.2.1]heptane was obtained as a yellow solid, 1.2 g, 56.3%, from 3-bromo-6-chloro-1-(tetrahydro-2H-pyran-2-yl)-1H-pyrazolo[4,3-c]pyridine and 2-oxa-5-azabicyclo[2.2.1]heptane following the procedure described in Preparation 513. ¹H NMR (500 MHz, CDCl₃) δ ppm 8.66 (d, J=2.5, Hz, 1H), 7.31 (d, J=8.5 Hz, 1H), 5.45-5.41 (m, 1H), 4.69-4.66 (m, 2H), 4.07-4.06 (m, 2H), 3.88-3.60 (m, 4H), 2.42-2.40 (m, 1H), 2.10-1.97 (m, 4H), 1.74-1.63 (m, 3H).

### Preparation 515: (3S)-1-(6-chloro-1-(tetrahydro-2H-pyran-2-yl)-1H-pyrazolo[4,3-c]pyridin-3-yl)pyrrolidine-3-carbonitrile

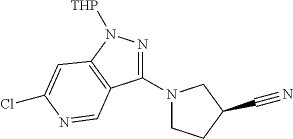

To a solution of (3S)-pyrrolidine-3-carbonitrile hydrochloride (365 mg, 2.75 mmol) in DMSO (3 mL) was added anhydrous K₂CO₃ (380 mg, 2.75 mmol), CuI (32 mg, 0.165 mmol) and DL-Proline (38 mg, 0.33 mmol) and the mixture stirred at rt for 5 min. 6-Chloro-3-iodo-1-(tetrahydro-2H-pyran-2-yl)-1H-pyrazolo[4,3-c]pyridine (200 mg, 0.55 mmol) was then added. The reaction mixture was purged with N₂, then heated at 80° C. for 6 h. The cooled mixture was diluted with water and extracted with EtOAc (×2). The combined organic extracts were washed with water (3×), brine, dried over MgSO₄, filtered, and concentrated in vacuo. The crude was purified by chromatography on silica gel (0-60% EtOAc-EtOH 3:1 with 2% NH₄OH in heptane) to give (3S)-1-(6-chloro-1-(tetrahydro-2H-pyran-2-yl)-1H-pyrazolo[4,3-c]pyridin-3-yl)pyrrolidine-3-carbonitrile (88 mg, 48% yield) as a white solid. LCMS m/z=332 [M+H]⁺

### Preparation 516: 6-chloro-3-(cyclopropylmethyl)-1-(tetrahydro-2H-pyran-2-yl)-1H-pyrazolo[4,3-c]pyridine

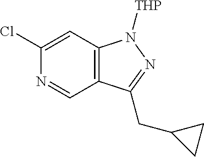

To a vial was added DMA (5.0 mL), pyridine-2,6-dicarboxamidine hydrochloride (9 mg, 39.5 μmol), followed by TEA (22 μL, 0.158 mmol) and the solution stirred for 5 min. NiCl₂ glyme (9 mg, 39.5 μmol), 3-bromo-6-chloro-1-tetrahydropyran-2-yl-pyrazolo[4,3-c]pyridine (250 mg, 0.79 mmol), bromomethylcyclopropane (213 mg, 1.58 mmol), NaI (30 mg, 0.197 mmol) and Zinc (103 mg, 1.58 mmol) were added and the reaction mixture was purged with N₂, then capped. The reaction mixture was heated at 60° C. overnight. The mixture was diluted with EtOAc, washed with water (3×), then brine. The organic layer was separated, dried and concentrated. The crude was purified by chromatography on silica gel (0-80% EtOAc in heptane) to give 6-chloro-3-(cyclopropylmethyl)-1-(tetrahydro-2H-pyran-2-yl)-1H-pyrazolo[4,3-c]pyridine (25 mg, 11% yield) as a colorless oil. LCMS m/z=292 [M+H]⁺

### Preparation 517: 6-chloro-3-(methylsulfonyl)-1-(tetrahydro-2H-pyran-2-yl)-1H-pyrazolo[4,3-c]pyridine

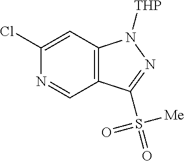

To a solution of sodium methanesulfinate (67 mg, 0.66 mmol) in DMSO (2 mL) was added anhydrous CuI (11 mg, 55.0 μmol), 2-(dimethylamino)acetic acid (11 mg, 0.11 mmol) and the solution stirred at rt for 5 min. 6-Chloro-3-iodo-1-tetrahydropyran-2-yl-pyrazolo[4,3-c]pyridine (200 mg, 0.55 mmol) was added, the reaction mixture was purged with N₂, then heated in microwave at 110° C. for 90 min. The cooled reaction mixture was poured into water and filtered. The precipitate was purified by chromatography on silica gel (0-80% EtOAc in heptane) to give 6-chloro-3-(methylsulfonyl)-1-(tetrahydro-2H-pyran-2-yl)-1H-pyrazolo[4,3-c]pyridine (49 mg, 28% yield). LCMS m/z=316 [M+H]⁺

### Preparation 518: N-(3-(6-oxa-3-azabicyclo[3.1.1]heptan-3-yl)-1-(tetrahydro-2H-pyran-2-yl)-1H-pyrazolo[4,3-c]pyridin-6-yl)acetamide

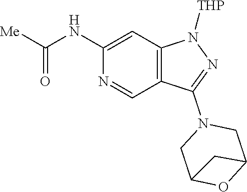

To a solution of 3-(6-chloro-1-(tetrahydro-2H-pyran-2-yl)-1H-pyrazolo[4,3-c]pyridin-3-yl)-6-oxa-3-azabicyclo[3.1.1]heptane (Preparation 513, 220 mg, 0.657 mmol) and acetamide (194.1 mg, 3.29 mmol) in dioxane (10 mL) was added Cs₂CO₃ (642.3 mg, 1.97 mmol) and BrettPhos Pd G₃ (59.57 mg, 0.066 mmol) and the reaction was stirred at 100° C. for 4 h. The mixture was filtered and concentrated under vacuum to give the crude, which was purified by column chromatography (MeOH in DCM from 0% to 8%) to give N-(3-(6-oxa-3-azabicyclo[3.1.1]heptan-3-yl)-1-(tetrahydro-2H-pyran-2-yl)-1H-pyrazolo[4,3-c]pyridin-6-yl)acetamide (200 mg, 85.2% yield) as yellow solid. LCMS m/z=358.2 [M+H]⁺

### Preparation 519: N-(3-(cyclopropylmethyl)-1-(tetrahydro-2H-pyran-2-yl)-1H-pyrazolo[4,3-c]pyridin-6-yl)acetamide

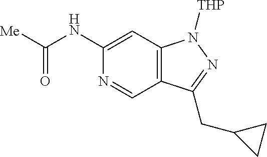

N-(3-(Cyclopropylmethyl)-1-(tetrahydro-2H-pyran-2-yl)-1H-pyrazolo[4,3-c]pyridin-6-yl)acetamide was obtained as a white powder, 23 mg 92% yield, from 6-chloro-3-(cyclopropylmethyl)-1-(tetrahydro-2H-pyran-2-yl)-1H-pyrazolo[4,3-c]pyridine (Preparation 516) and acetamide, following a similar procedure to that described in Preparation 518. LCMS m/z=315 [M+H]⁺

### Preparation 520: methyl (3-(6-oxa-3-azabicyclo[3.1.1]heptan-3-yl)-1-(tetrahydro-2H-pyran-2-yl)-1H-pyrazolo[4,3-c]pyridin-6-yl)carbamate

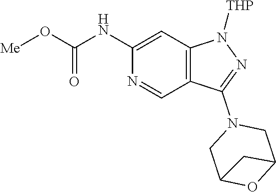

To solution of 3-(6-chloro-1-(tetrahydro-2H-pyran-2-yl)-1H-pyrazolo[4,3-c]pyridin-3-yl)-6-oxa-3-azabicyclo[3.1.1]heptane (Preparation 513, 500 mg, 1.49 mmol) in dioxane (150 mL) was added methyl carbamate (560.5 mg, 7.47 mmol), Cs₂CO₃ (1.5 g, 4.48 mmol) and BrettPhos Pd G₃ (135.4 mg, 0.149 mmol) and the reaction mixture stirred at 100° C. for 12 h. The reaction was slowly quenched with H₂O (50 mL), the layers were separated and the aqueous layer extracted with EtOAc (50 mL×3). The combined organic layers were washed with brine (25 mL×3), dried over Na₂SO₄, filtered and concentrated. The residue was purified with silica gel chromatography (ISCO®; EtOAc) to give methyl (3-(6-oxa-3-azabicyclo[3.1.1]heptan-3-yl)-1-(tetrahydro-2H-pyran-2-yl)-1H-pyrazolo[4,3-c]pyridin-6-yl)carbamate (355 mg, 63.7% yield) as a yellow solid. LCMS m/z=374.2 [M+H]⁺

### Preparations 521 to 525

The compounds in the following table were prepared from the appropriate chloro pyrazolo[4,3-c]pyridine and amide, following a similar procedure to that described in Preparation 520.

| Prep- | |
|---|---|
| aration | |
| No | Name, Structure, Starting Material (SM), Yield, Data |
| 521 | N-(3-(6-oxa-3-azabicyclo[3.1.1]heptan-3-yl)-1-(tetrahydro- |
| | 2H-pyran-2-yl)-1H-pyrazolo[4,3-c]pyridin-6-yl)propionamide |
| | 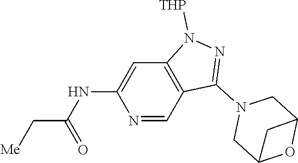 |
| | SM: 3-(6-chloro-1-(tetrahydro-2H-pyran-2-yl)-1H- |
| | pyrazolo[4,3-c]pyridin-3-yl)-6-oxa-3-azabicyclo[3.1.1] |
| | heptane (Preparation 513) |
| | white solid, 313.6 mg, 94.2% yield. 1H NMR (400 MHz, |
| | CDCl₃) δ ppm 8.71 (s, 1H), 8.22 (s, 1H), 8.07 (br s, 1H), |
| | 5.51-5.48 (m, 1H), 4.76 (d, J = 6.4 Hz, 2H), 4.13-4.07 |
| | (m, 2H), 4.02-3.99 (m, 1H), 3.93-3.88 (m, 3H), 3.72- |
| | 3.67 (m, 1H), 3.31-3.29 (m, 1H), 2.49-2.43 (m, 3H), |
| | 2.14-2.04 (m, 4H), 1.94-1.91 (m, 1H), 1.30-1.24 (m, 3H). |
| 522 | N-(3-((S)-3-cyanopyrrolidin-1-yl)-1-(tetrahydro-2H-pyran- |
| | 2-yl)-1H-pyrazolo[4,3-c]pyridin-6-yl)acetamide |
| | 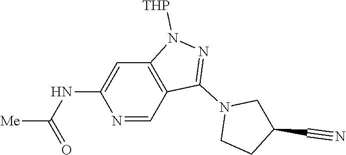 |
| | SM: (3S)-1-(6-chloro-1-(tetrahydro-2H-pyran-2-yl)-1H- |
| | pyrazolo[4,3-c]pyridin-3-yl)pyrrolidine-3-carbonitrile |
| | (Preparation 515) light yellow powder, 86 mg, 94% yield. |
| | LCMS m/z = 355 [M + H]⁺ |
| 523 | N-(3-(6-oxa-3-azabicyclo[3.1.1]heptan-3-yl)-1- |
| | (tetrahydro-2H-pyran-2-yl)-1H-pyrazolo[4,3-c]pyridin- |
| | 6-yl)formamide |
| | 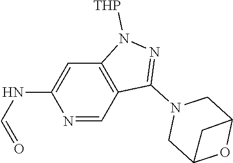 |
| | SM: 3-(6-chloro-1-(tetrahydro-2H-pyran-2-yl)-1H-pyrazolo |
| | [4,3-c]pyridin-3-yl)-6-oxa-3-azabicyclo[3.1.1]heptane |
| | (Preparation 513) white solid, 291 mg, 48.5% yield. ¹H NMR |
| | (500 MHz, CDCl₃): δ ppm 8.75 (s, 1H), 8.52 (s, 2H), 8.19 |
| | (s, 1H), 5.52-5.50 (m, 1H), 5.77 (d, J = 6.0 Hz, 2H), 4.10-4.08 |
| | (m, 2H), 4.02-4.00 (m, 2H), 3.90-3.88 (m, 2H), 3.77-3.75 (m, |
| | 2H), 3.30-3.22 (m, 2H), 2.56-2.52 (m, 2H), 2.00-1.93 (m, 2H) |
| 524 | N-(3-(2-oxa-5-azabicyclo[2.2.1]heptan-5-yl)-1- |
| | (tetrahydro-2H-pyran-2-yl)-1H-pyrazolo[4,3-c]pyridin-6- |
| | yl)acetamide |
| | 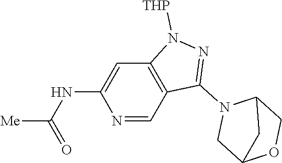 |
| | SM: 5-(6-chloro-1-(tetrahydro-2H-pyran-2-yl)-1H-pyrazolo |
| | [4,3-c]pyridin-3-yl)-2-oxa-5-azabicyclo[2.2.1]heptane |
| | (Preparation 514) yellow solid, 1.1 g, 86.6% yield. ¹H NMR |
| | (500 MHz, CDCl₃) δ ppm 8.55 (d, J = 5.0 Hz, 1H), |
| | 8.19-8.15 (m, 2H), 5.47 (d, J = 9.5 Hz, 1H), 4.71- |
| | 4.68 (m, 2H), 4.09-4.06 (m, 2H), 3.87-3.85 (m, 1H), 3.79-3.73 |
| | (m, 2H), 3.63-3.61 (m, 1H), 2.50-2.48 (m, 1H), 2.09-2.01 |
| | (m, 4H), 1.97-1.91 (m, 2H), 1.77-1.70 (m, 4H). |
| 525 | N-(3-(methylsulfonyl)-1-(tetrahydro-2H-pyran-2-yl)-1H- |
| | pyrazolo[4,3-c]pyridin-6-yl)acetamide |
| | 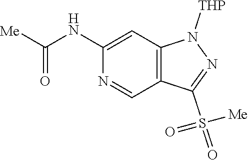 |
| | SM: 6-chloro-3-(methylsulfonyl)-1-(tetrahydro-2H-pyran-2- |
| | yl)-1H-pyrazolo[4,3-c]pyridine (Preparation 517) |
| | 26 mg, 53% yield. LCMS m/z = 339 [M + H]⁺ |

### Preparation 526: N-(3-morpholino-1-(tetrahydro-2H-pyran-2-yl)-1H-pyrazolo[4,3-c]pyridin-6-yl)acetamide

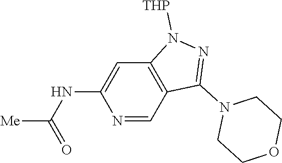

A mixture of N-(3-iodo-1-(tetrahydro-2H-pyran-2-yl)-1H-pyrazolo[4,3-c]pyridin-6-yl)acetamide (Preparation 12, 100 mg, 0.259 mmol), Cs₂CO₃ (169 mg, 0.518 mmol), Xantphos Pd G4 (25 mg, 25.89 μmol) and morpholine (113 mg, 1.29 mmol) in dioxane (2 mL) was purged with N₂ and the reaction was heated at 80° C. overnight. The resulting solid was filtered off and the crude was purified by chromatography on silica gel (0-80% EtOAc-EtOH 3:1 with 20% NH₄OH in heptane) to give N-(3-morpholino-1-(tetrahydro-2H-pyran-2-yl)-1H-pyrazolo[4,3-c]pyridin-6-yl)acetamide (18 mg, 18% yield) as a white solid. LCMS m/z=346 [M+H]⁺

### Preparation 527: N-(3-(3-(dimethylamino)-3-methylpyrrolidin-1-yl)-1-(tetrahydro-2H-pyran-2-yl)-1H-pyrazolo[4,3-c]pyridin-6-yl)acetamide

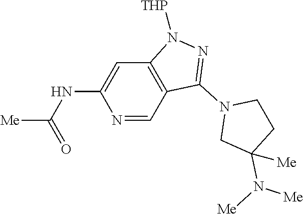

A suspension of N,N,3-trimethylpyrrolidin-3-amine (5.36 g, 26.67 mmol, Hydrochloride), N-(3-iodo-1-(tetrahydro-2H-pyran-2-yl)-1H-pyrazolo[4,3-c]pyridin-6-yl)acetamide (Preparation 12, 5.15 g, 13.34 mmol), 2-(2,6-difluoroanilino)-2-oxo-acetic acid (536.4 mg, 2.67 mmol) and K₃PO₄ (14.15 g, 66.68 mmol) in dry DMSO (60 mL) was sparged with N₂ for 5 min. CuI (508 mg, 2.67 mmol) was added and the reaction was stirred at 100° C. for 17 h. The cooled mixture was diluted with water and EtOAc, filtered through a pad of Celite®, the layers separated and the aqueous extracted with EtOAc (3×). The combined organic layers were washed with brine, dried (MgSO₄), filtered and concentrated in vacuo. The residue was purified by silica gel chromatography (0-10% MeOH in DCM) to give. N-(3-(3-(dimethylamino)-3-methylpyrrolidin-1-yl)-1-(tetrahydro-2H-pyran-2-yl)-1H-pyrazolo[4,3-c]pyridin-6-yl)acetamide (4.48 g, 86.9% yield) was obtained as an orange foam. LCMS m/z=387.1 [M+H]⁺

### Preparation 528: N-(3-(2-cyanocyclopropyl)-1-(tetrahydro-2H-pyran-2-yl)-1H-pyrazolo[4,3-c]pyridin-6-yl)acetamide

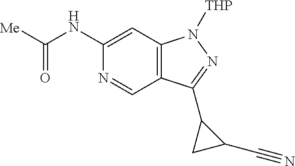

To a solution of N-(3-iodo-1-(tetrahydro-2H-pyran-2-yl)-1H-pyrazolo[4,3-c]pyridin-6-yl)acetamide (Preparation 12, 500 mg, 1.29 mmol) and 2-(4,4,5,5-tetramethyl-1,3,2-dioxaborolan-2-yl)cyclopropanecarbonitrile (500 mg, 2.59 mmol) in toluene (12 mL) was added Cs₂CO₃ (2 M in water, 1.29 mL), Pd(OAc)₂ (15 mg, 64.7 μmol) and cataCXium A (23 mg, 64.7 μmol). The mixture was heated at 90° C. for 16 h. The crude was diluted with EtOAc and washed with aq. NaHCO₃. The organic layer was then dried and concentrated. The crude was purified by chromatography on silica gel (0-70% EtOAc-EtOH 3:1 with 2% NH₄OH in heptane) to give N-(3-(2-cyanocyclopropyl)-1-(tetrahydro-2H-pyran-2-yl)-1H-pyrazolo[4,3-c]pyridin-6-yl)acetamide (325 mg, 77% yield) as a white powder. LCMS m/z=326 [M+H]⁺

### Preparation 529: N-(1-(tetrahydro-2H-pyran-2-yl)-3-(tetrahydro-2H-pyran-4-yl)-1H-pyrazolo[4,3-c]pyridin-6-yl)acetamide

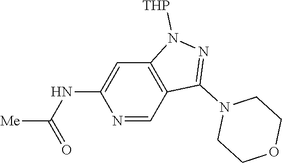

A mixture of N-(3-iodo-1-(tetrahydro-2H-pyran-2-yl)-1H-pyrazolo[4,3-c]pyridin-6-yl)acetamide (Preparation 12, 200 mg, 0.518 mmol), Pd(OAc)₂ (12 mg, 0.052 mmol) and SPhos (32 mg, 0.078 mmol) in 2-methyltetrahydrofuran (4 mL) was purged with N₂. Bromo(tetrahydropyran-4-yl)zinc (0.5 M, 3.11 mL) was then added and the resulting dark suspension was stirred at rt for 2 h. The reaction was quenched with water/EtOAc and the layers separated. The organic phase was dried and concentrated in vacuo. The crude was purified by chromatography on silica gel (0-70% EtOAc in heptane) to give N-(1-(tetrahydro-2H-pyran-2-yl)-3-(tetrahydro-2H-pyran-4-yl)-1H-pyrazolo[4,3-c]pyridin-6-yl)acetamide (109 mg, 61% yield) as a white powder. LCMS m/z=345 [M+H]⁺

### Preparation 530: tert-butyl 3-(6-acetamido-1-(tetrahydro-2H-pyran-2-yl)-1H-pyrazolo[4,3-c]pyridin-3-yl)azetidine-1-carboxylate

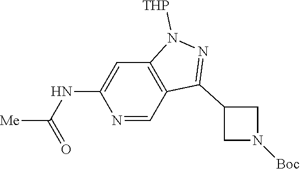

A mixture of N-(3-iodo-1-(tetrahydro-2H-pyran-2-yl)-1H-pyrazolo[4,3-c]pyridin-6-yl)acetamide (Preparation 12, 500 mg, 1.29 mmol) and dichloro[bis(diphenylphosphinophenyl)ether]palladium(II), (46 mg, 64.74 μmol) in THF (5 mL) was purged with N₂. (1-tert-Butoxycarbonylazetidin-3-yl)-iodo-zinc (0.5 M, 7.77 mL) was then added slowly at 0° C. and the resulting dark suspension was stirred at rt for 2 h. The reaction was quenched with MeOH. The crude was purified by chromatography on silica gel (0-70% EtOAc in heptane) to give tert-butyl 3-(6-acetamido-1-tetrahydropyran-2-yl-pyrazolo[4,3-c]pyridin-3-yl)azetidine-1-carboxylate (458 mg, 85% yield) as a white powder. LCMS m/z=416 [M+H]⁺

### Preparation 531: N-(1-(tetrahydro-2H-pyran-2-yl)-3-(3,3,3-trifluoroprop-1-en-2-yl)-1H-pyrazolo[4,3-c]pyridin-6-yl)acetamide

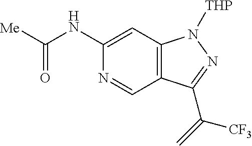

To a solution of N-(3-iodo-1-(tetrahydro-2H-pyran-2-yl)-1H-pyrazolo[4,3-c]pyridin-6-yl)acetamide (Preparation 12, 300 mg, 0.78 mmol) and 4,4,5,5-tetramethyl-2-[1-(trifluoromethyl)vinyl]-1,3,2-dioxaborolane (345 mg, 1.55 mmol) in dioxane (6 mL) was added aq. K₂CO₃ (2 M, 777 μL) and bis(tricyclohexylphosphine)palladium(0) (26 mg, 38.8 μmol) and the reaction was heated at 100° C. for 4 h. The cooled mixture was diluted with EtOAc and washed with brine. The organic layer was dried and concentrated. The crude was purified by chromatography on silica gel (0-100% EtOAc in heptane) to give N-(1-(tetrahydro-2H-pyran-2-yl)-3-(3,3,3-trifluoroprop-1-en-2-yl)-1H-pyrazolo[4,3-c]pyridin-6-yl)acetamide (181 mg, 65% yield). LCMS m/z=355 [M+H]⁺

### Preparation 532: N-(1-(tetrahydro-2H-pyran-2-yl)-3-(1,1,1-trifluoropropan-2-yl)-1H-pyrazolo[4,3-c]pyridin-6-yl)acetamide

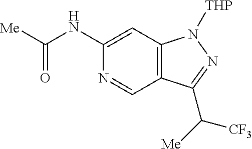

To a solution of N-(1-(tetrahydro-2H-pyran-2-yl)-3-(3,3,3-trifluoroprop-1-en-2-yl)-1H-pyrazolo[4,3-c]pyridin-6-yl)acetamide (Preparation 531, 170 mg, 0.48 mmol) in EtOAc (5 mL) under N₂ was added Pd/C (1.02 g, 0.48 mmol, 5% purity). The mixture was purged with H₂ and stirred under a H₂ balloon at rt overnight. The mixture was filtered, the filtrate was concentrated and purified by chromatography on silica gel (0-100% EtOAc in heptane) to give N-(1-(tetrahydro-2H-pyran-2-yl)-3-(1,1,1-trifluoropropan-2-yl)-1H-pyrazolo[4,3-c]pyridin-6-yl)acetamide (104 mg, 60% yield) as a white powder. LCMS m/z=357 [M+H]⁺

### Preparation 533: phenyl 6-acetamido-1-trityl-1H-pyrazolo[4,3-c]pyridine-3-carboxylate

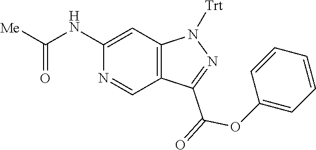

TEA (320 μL, 2.30 mmol) was added to a mixture of N-(3-iodo-1-trityl-1H-pyrazolo[4,3-c]pyridin-6-yl)acetamide (Preparation 4, 500 mg, 0.919 mmol), phenyl formate (250 μL, 2.30 mmol) and Xantphos Pd G4 (53 mg, 55.11 μmol) in MeCN (5 mL) at rt, the reaction mixture purged with N₂, then stirred at 80° C. overnight. After cooling to rt, the mixture was diluted with EtOAc, washed with water, then brine. The organic layer was separated, dried and concentrated. The crude was purified by chromatography on silica gel (0-100% EtOAc in heptane) to obtain phenyl 6-acetamido-1-trityl-pyrazolo[4,3-c]pyridine-3-carboxylate (263 mg, 53% yield) as a white solid. LCMS m/z=539 [M+H]⁺

### Preparation 534: N-(3-(hydroxymethyl)-1-trityl-1H-pyrazolo[4,3-c]pyridin-6-yl)acetamide

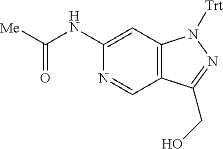

To a solution of phenyl 6-acetamido-1-trityl-pyrazolo[4,3-c]pyridine-3-carboxylate (Preparation 533, 260 mg, 0.483 mmol) in THF (5 mL) was added LAH (1 M, 965 μL) at 0° C. and the reaction stirred at 0° C. for 1 h. The reaction was quenched with Na₂SO₄·10H₂O/Celite® and the mixture stirred at rt for 10 min. The mixture was filtered and the filtrate was concentrated and purified by HPLC to give N-(3-(hydroxymethyl)-1-trityl-1H-pyrazolo[4,3-c]pyridin-6-yl)acetamide (70 mg, 32% yield) as a white powder. LCMS m/z=449 [M+H]⁺

### Preparation 535: N-(3-formyl-1-trityl-1H-pyrazolo[4,3-c]pyridin-6-yl)acetamide

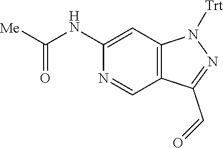

To a mixture of N-(3-(hydroxymethyl)-1-trityl-1H-pyrazolo[4,3-c]pyridin-6-yl)acetamide (Preparation 534, 60 mg, 0.134 mmol) in DCM (1 mL) was added Dess-Martin periodinane (68 mg, 0.161 mmol) and the reaction stirred at rt for 1 h. 1N NaOH (1 mL) was added, and the solution stirred for 5 min. The organic layer was separated and the aqueous layer was extracted with DCM (3×). The combined organic layer was dried and concentrated to give N-(3-formyl-1-trityl-1H-pyrazolo[4,3-c]pyridin-6-yl)acetamide (48 mg, 80% yield) as a white powder. LCMS m/z=447 [M+H]⁺

### Preparation 536: N-(3-(difluoromethyl)-1-trityl-1H-pyrazolo[4,3-c]pyridin-6-yl)acetamide

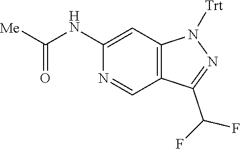

N-(3-(Difluoromethyl)-1-trityl-1H-pyrazolo[4,3-c]pyridin-6-yl)acetamide was obtained as a light yellow oil, 48 mg, 97%, from N-(3-formyl-1-trityl-1H-pyrazolo[4,3-c]pyridin-6-yl)acetamide (Preparation 535), following a similar procedure to that described in Preparation 459. LCMS m/z=469 [M+H]⁺

### Preparation 537: N-(1-trityl-3-vinyl-1H-pyrazolo[4,3-c]pyridin-6-yl)acetamide

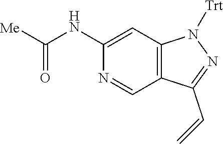

A mixture of N-(3-iodo-1-trityl-1H-pyrazolo[4,3-c]pyridin-6-yl)acetamide (Preparation 4, 400 mg, 0.735 mmol), 4,4,5,5-tetramethyl-2-vinyl-1,3,2-dioxaborolane (566 mg, 3.67 mmol), aq. K₂CO₃ (2 M, 1.10 mL) and Pd(PCy₃)₂ (25 mg, 36.7 μmol) in dioxane (7 mL) was purged with N₂. The mixture was then sealed and heated at 80° C. for 2 h. The cooled mixture was diluted with EtOAc, washed with water (3×), then brine. The organic layer was separated, dried and concentrated. The crude was purified by chromatography on silica gel (0-80% EtOAc in heptane) to give N-(1-trityl-3-vinyl-pyrazolo[4,3-c]pyridin-6-yl)acetamide (278 mg, 85% yield) as an off-white powder. LCMS m/z=445 [M+H]⁺

### Preparation 538: N-(3-(1,2-dihydroxyethyl)-1-trityl-1H-pyrazolo[4,3-c]pyridin-6-yl)acetamide

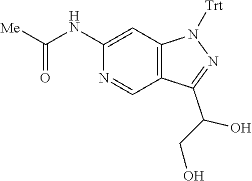

To a solution of N-(1-trityl-3-vinyl-1H-pyrazolo[4,3-c]pyridin-6-yl)acetamide (Preparation 537, 275 mg, 0.619 mmol) in tert-butanol (5 mL) was added 4-methyl-4-oxido-morpholin-4-ium (152 mg, 1.30 mmol) and water (2 mL), followed by tetraoxoosmium (0.078 M, 397 μL) and the reaction was stirred at rt overnight. The reaction was diluted with water and extracted with EtOAc. The combined organic layer was dried and concentrated to give N-(3-(1,2-dihydroxyethyl)-1-trityl-1H-pyrazolo[4,3-c]pyridin-6-yl)acetamide (276 mg, 93% yield) as a white powder. LCMS m/z=479 [M+H]⁺

### Preparation 539: N-(3-(1,2-difluoroethyl)-1-trityl-1H-pyrazolo[4,3-c]pyridin-6-yl)acetamide

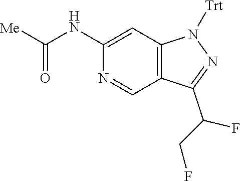

N-(3-(1,2-Difluoroethyl)-1-trityl-1H-pyrazolo[4,3-c]pyridin-6-yl)acetamide was obtained as a white powder, 24 mg, 15% yield, from N-(3-(1,2-dihydroxyethyl)-1-trityl-1H-pyrazolo[4,3-c]pyridin-6-yl)acetamide (Preparation 538) following a similar procedure, using 2 equivalents of DAST, to that described in Preparation 442. LCMS m/z=483 [M+H]⁺

### Preparation 540: N-(3-isopropyl-1-trityl-1H-pyrazolo[4,3-c]pyridin-6-yl)acetamide

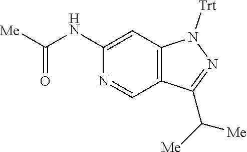

Bromo(isopropyl)zinc (0.5 M, 1.47 mL) was added to a mixture of N-(3-iodo-1-trityl-pyrazolo[4,3-c]pyridin-6-yl)acetamide (Preparation 4, 200 mg, 0.367 mmol), Pd(OAc)₂ (8 mg, 36.74 μmol) and 2-dicyclohexylphosphino-2,6-dimethoxybiphenyl (23 mg, 55.1 μmol) in 2-methyltetrahydrofuran (3 mL) and the resulting dark suspension was stirred at rt for 2 h.

The reaction was then quenched with water and EtOAc. The organic layer was separated, dried and concentrated. The crude was purified by chromatography on silica gel (0-70% EtOAc in heptane) to give N-(3-isopropyl-1-trityl-1H-pyrazolo[4,3-c]pyridin-6-yl)acetamide (137 mg, 80% yield) as a white powder. LCMS m/z=461 [M+H]⁺

### Preparation 541: N-(3-methoxy-1-(tetrahydro-2H-pyran-2-yl)-1H-pyrazolo[4,3-c]pyridin-6-yl)acetamide

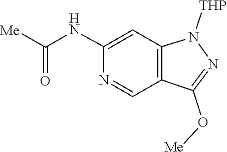

A mixture of N-(3-iodo-1-tetrahydropyran-2-yl-pyrazolo[4,3-c]pyridin-6-yl)acetamide (Preparation 12, 100 mg, 0.259 mmol), Cs₂CO₃ (169 mg, 0.518 mmol), tBuBrettPhos Pd G3 (22 mg, 0.026 mmol), and MeOH (105 μL) in dioxane (2 mL) was purged with N₂. The reaction was heated at 80° C. for 2 h. The cooled mixture was filtered and the filtrate was purified by chromatography on silica gel (0-80% EtOAc-EtOH 3:1 with 2% NH₄OH in heptane) to give N-(3-methoxy-1-tetrahydropyran-2-yl-pyrazolo[4,3-c]pyridin-6-yl)acetamide (71 mg, 85% yield). LCMS m/z=291 [M+H]⁺

### Preparation 542: N-(3-(3-methoxycyclobutoxy)-1-(tetrahydro-2H-pyran-2-yl)-1H-pyrazolo[4,3-c]pyridin-6-yl)acetamide

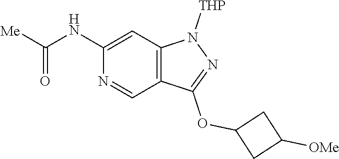

N-(3-(3-Methoxycyclobutoxy)-1-(tetrahydro-2H-pyran-2-yl)-1H-pyrazolo[4,3-c]pyridin-6-yl)acetamide was obtained as a light-yellow oil, from N-(3-iodo-1-trityl-1H-pyrazolo[4,3-c]pyridin-6-yl)acetamide (Preparation 4) and 3-methoxycyclobutanol, following the procedure described in Preparation 541. LCMS m/z=361 [M+H]⁺

### Preparation 543: N-(3-(6-oxa-3-azabicyclo[3.1.1]heptan-3-yl)-1H-pyrazolo[4,3-c]pyridin-6-yl)acetamide

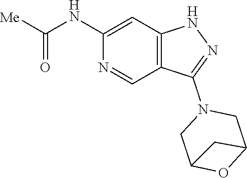 [SMILES: CC(=O)Nc1cc2[nH]nc(N3CC4CC(C3)O4)c2cn1]

To a solution of N-(3-(6-oxa-3-azabicyclo[3.1.1]heptan-3-yl)-1-(tetrahydro-2H-pyran-2-yl)-1H-pyrazolo[4,3-c]pyridin-6-yl)acetamide (Preparation 518, 100 mg, 0.28 mmol) in HFIPA (3 mL) was added TFA (255.2 mg, 2.24 mmol) and the reaction mixture was stirred at 20° C. for 10 h. The reaction was added to aq. NaHCO₃ (30 mL) and extracted with DCM (30 mL×2). The combined organic phase was washed with water (20 mL), dried over Na₂SO₄, filtered and concentrated to give N-(3-(6-oxa-3-azabicyclo[3.1.1]heptan-3-yl)-1H-pyrazolo[4,3-c]pyridin-6-yl)acetamide (65 mg, crude) as yellow oil. LCMS m/z=274.1 [M+H]⁺

### Preparation 544: methyl (3-(6-oxa-3-azabicyclo[3.1.1]heptan-3-yl)-1H-pyrazolo[4,3-c]pyridin-6-yl)carbamate

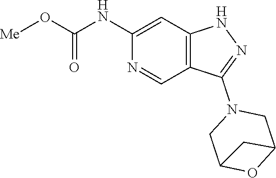 [SMILES: COC(=O)Nc1cc2[nH]nc(N3CC4CC(C3)O4)c2cn1]

To a solution of methyl (3-(6-oxa-3-azabicyclo[3.1.1]heptan-3-yl)-1-(tetrahydro-2H-pyran-2-yl)-1H-pyrazolo[4,3-c]pyridin-6-yl)carbamate (Preparation 520, 355 mg, 0.95 mmol) in HFiPA (3 mL) was added TFA (1.8 g, 16.23 mmol) and the reaction stirred at 25° C. for 5 h.

The pH of the mixture was adjusted to 7-8 with NH₄OH and concentrated under reduced pressure to give methyl (3-(6-oxa-3-azabicyclo[3.1.1]heptan-3-yl)-1H-pyrazolo[4,3-c]pyridin-6-yl)carbamate (340 mg, crude). LCMS m/z=290.1 [M+H]⁺

### Preparation 545: N-(3-(6-oxa-3-azabicyclo[3.1.1]heptan-3-yl)-1H-pyrazolo[4,3-c]pyridin-6-yl)propionamide

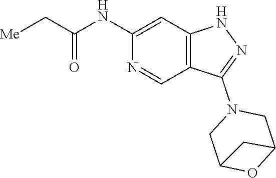 [SMILES: CCC(=O)Nc1cc2[nH]nc(N3CC4CC(C3)O4)c2cn1]

N-(3-(6-Oxa-3-azabicyclo[3.1.1]heptan-3-yl)-1H-pyrazolo[4,3-c]pyridin-6-yl)propionamide was obtained as a yellow gum, from N-(3-(6-oxa-3-azabicyclo[3.1.1]heptan-3-yl)-1-(tetrahydro-2H-pyran-2-yl)-1H-pyrazolo[4,3-c]pyridin-6-yl)propionamide (Preparation 521), following the procedure described in Preparation 545. LCMS m/z=288.1 [M+H]⁺

### Preparation 546: N-(3-morpholino-1H-pyrazolo[4,3-c]pyridin-6-yl)acetamide

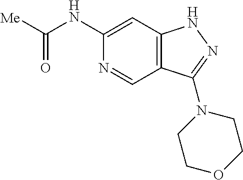 [SMILES: CC(=O)Nc1cc2[nH]nc(N3CCOCC3)c2cn1]

To a solution of N-(3-morpholino-1-(tetrahydro-2H-pyran-2-yl)-1H-pyrazolo[4,3-c]pyridin-6-yl)acetamide (Preparation 526, 18 mg, 52.11 μmol) in DCM (1.0 mL) was added TFA (0.1 mL) and the mixture was stirred at rt overnight. The reaction mixture was concentrated under vacuum, then diluted with EtOAc, washed with aq. NaHCO₃. The organic layer was separated, dried and concentrated to give N-(3-morpholino-1H-pyrazolo[4,3-c]pyridin-6-yl)acetamide (12 mg, 88% yield) as an off-white solid. LCMS m/z=262 [M+H]⁺

### Preparations 547 to 556

The compounds in the following table were prepared from the appropriate protected pyrazolo[4,3-c]pyridine, following a similar procedure to that described in Preparation 546.

| Preparation | |
|---|---|
| No | Name, Structure, Starting Materials (SM), Yield, Data |
| 547 | N-(3-(3-(dimethylamino)-3-methylpyrrolidin-1-yl)- |
| | 1H-pyrazolo[4,3-c]pyridin-6-yl)acetamide |
| | 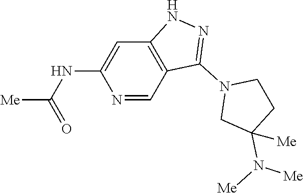 [SMILES: CC(=O)Nc1cc2[nH]nc(N3CCC(C)(N(C)C)C3)c2cn1] |
| | SM: N-(3-(3-(dimethylamino)-3-methylpyrrolidin-1-yl)- |
| | 1-(tetrahydro-2H-pyran-2-yl)-1H-pyrazolo[4,3-c] |
| | pyridin-6-yl)acetamide (Preparation 527) |
| | pale yellow solid, LCMS m/z = 303.1 [M + H]⁺ |
| 548 | N-(3-(1,2-difluoroethyl)-1H-pyrazolo[4,3-c]pyridin-6- |
| | yl)acetamide |
| | 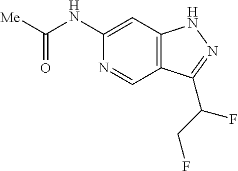 [SMILES: CC(=O)Nc1cc2[nH]nc(C(F)CF)c2cn1] |
| | SM: N-(3-(1,2-difluoroethyl)-1-trityl-1H-pyrazolo[4,3-c] |
| | pyridin-6-yl)acetamide (Preparation 539) |
| | light yellow powder. LCMS m/z = 241 [M + H]⁺. |
| 549 | N-(3-(cyclopropylmethyl)-1H-pyrazolo[4,3-c]pyridin- |
| | 6-yl)acetamide |
| | 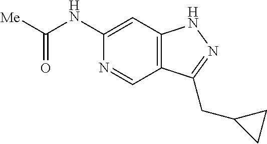 [SMILES: CC(=O)Nc1cc2[nH]nc(CC3CC3)c2cn1] |
| | SM: N-(3-(cyclopropylmethyl)-1-(tetrahydro-2H-pyran- |
| | 2-yl)-1H-pyrazolo[4,3-c]pyridin-6-yl)acetamide |
| | (Preparation 519) 25 mg, 99% yield as a colorless oil. |
| | LCMS m/z = 231 [M + H]⁺ |
| 550 | N-(3-(2-cyanocyclopropyl)-1H-pyrazolo[4,3-c] |
| | pyridin-6-yl)acetamide |
| | 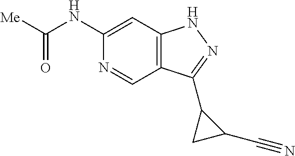 [SMILES: CC(=O)Nc1cc2[nH]nc(C3CC3C#N)c2cn1] |
| | SM: N-(3-(2-cyanocyclopropyl)-1-(tetrahydro-2H-pyran- |
| | 2-yl)-1H-pyrazolo[4,3-c]pyridin-6-yl)acetamide |
| | (Preparation 528). LCMS m/z = 242 [M + H]⁺ |
| 551 | N-(3-(tetrahydro-2H-pyran-4-yl)-1H-pyrazolo[4,3-c] |
| | pyridin-6-yl)acetamide |
| | 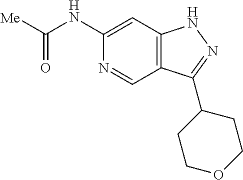 [SMILES: CC(=O)Nc1cc2[nH]nc(C3CCOCC3)c2cn1] |
| | SM: N-(1-(tetrahydro-2H-pyran-2-yl)-3-(tetrahydro-2H- |
| | pyran-4-yl)-1H-pyrazolo[4,3-c]pyridin-6-yl)acetamide |
| | (Preparation 529) a white powder, 109 mg, 95%. LCMS |
| | m/z = 261 [M + H]⁺ |
| 552 | N-(3-(difluoromethyl)-1H-pyrazolo[4,3-c]pyridin- |
| | 6-yl)acetamide |
| | 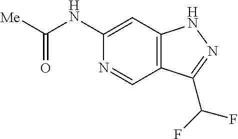 [SMILES: CC(=O)Nc1cc2[nH]nc(C(F)F)c2cn1] |
| | SM: N-(3-(difluoromethyl)-1-trityl-1H-pyrazolo[4,3-c] |
| | pyridin-6-yl)acetamide (Preparation 536) |
| | white solid, LCMS m/z = 227 [M + H]⁺ |
| 553 | N-(3-(1,1,1-trifluoropropan-2-yl)-1H-pyrazolo[4,3-c] |
| | pyridin-6-yl)acetamide |
| | 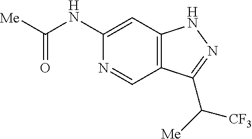 [SMILES: CC(=O)Nc1cc2[nH]nc(C(C)C(F)(F)F)c2cn1] |
| | SM: N-(1-(tetrahydro-2H-pyran-2-yl)-3-(1,1,1-trifluoro- |
| | propan-2-yl)-1H-pyrazolo[4,3-c]pyridin-6-yl)acetamide |
| | (Preparation 532) light yellow solid. LCMS m/z = 273 |
| | [M + H]⁺ |
| 554 | N-(3-methoxy-1H-pyrazolo[4,3-c]pyridin-6- |
| | yl)acetamide |
| | 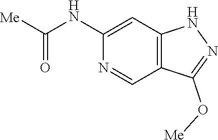 [SMILES: COc1n[nH]c2cc(NC(C)=O)ncc12] |
| | SM: N-(3-methoxy-1-(tetrahydro-2H-pyran-2-yl)-1H- |
| | pyrazolo[4,3-c]pyridin-6-yl)acetamide (Preparation 541) |
| | LCMS m/z = 207 [M + H]⁺ |
| 555 | N-(3-(3-methoxycyclobutoxy)-1H-pyrazolo[4,3-c] |
| | pyridin-6-yl)acetamide |
| | 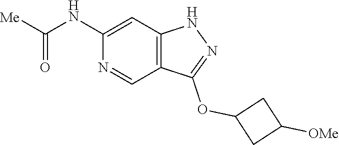 [SMILES: COC1CC(Oc2n[nH]c3cc(NC(C)=O)ncc23)C1] |
| | SM: N-(3-(3-methoxycyclobutoxy)-1-(tetrahydro-2H- |
| | pyran-2-yl)-1H-pyrazolo[4,3-c]pyridin-6-yl)acetamide |
| | (Preparation 542) |
| 556 | N-(3-(methylsulfonyl)-1H-pyrazolo[4,3-c]pyridin- |
| | 6-yl)acetamide |
| | 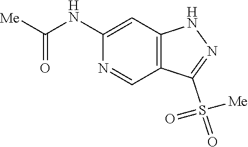 [SMILES: CC(=O)Nc1cc2[nH]nc(S(C)(=O)=O)c2cn1] |
| | SM: N-(3-(methylsulfonyl)-1-(tetrahydro-2H-pyran- |
| | 2-yl)-1H-pyrazolo[4,3-c]pyridin-6-yl)acetamide |
| | (Preparation 525) 38 mg, 74% colorless oil. |
| | LCMS m/z = 255 [M + H]⁺ |

### Preparation 557: N-(3-isopropyl-1H-pyrazolo[4,3-c]pyridin-6-yl)acetamide

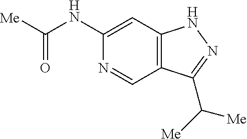 [SMILES: CC(=O)Nc1cc2[nH]nc(C(C)C)c2cn1]

To a solution of N-(3-isopropyl-1-trityl-1H-pyrazolo[4,3-c]pyridin-6-yl)acetamide (Preparation 540, 400 mg, 0.868 mmol) in DCM (6 mL) was added TFA (600 μL) and the reaction stirred at rt for 2 h. The mixture was concentrated in vacuo, the residue diluted with EtOAc and neutralized with aq. Na₂CO₃. The organic layer was separated, dried and concentrated. The solid formed was washed with heptane, then dried to give N-(3-isopropyl-1H-pyrazolo[4,3-c]pyridin-6-yl)acetamide (159 mg, 83% yield) as a white solid. LCMS m/z=219 [M+H]⁺

### Preparation 558: (S)—N-(3-(3-cyanopyrrolidin-1-yl)-1H-pyrazolo[4,3-c]pyridin-6-yl)acetamide trifluoroacetate

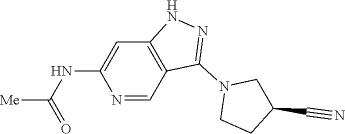 [SMILES: CC(=O)Nc1cc2[nH]nc(N3CC[C@H](C#N)C3)c2cn1]

To a solution of N-(3-((S)-3-cyanopyrrolidin-1-yl)-1-(tetrahydro-2H-pyran-2-yl)-1H-pyrazolo[4,3-c]pyridin-6-yl)acetamide (Preparation 552, 80 mg, 0.226 mmol) in DCM (2 mL) was added TFA (0.4 mL) and the reaction was stirred at 40° C. for 2 h. The reaction mixture was concentrated under vacuum to give (S)—N-(3-(3-cyanopyrrolidin-1-yl)-1H-pyrazolo[4,3-c]pyridin-6-yl)acetamide trifluoroacetate (166 mg) as a brownish oil. LCMS m/z=271 [M+H]⁺

### Preparation 559: N-(3-(azetidin-3-yl)-1H-pyrazolo[4,3-c]pyridin-6-yl)acetamide trifluoroacetate

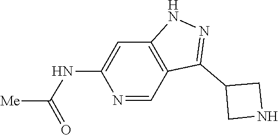 [SMILES: CC(=O)Nc1cc2[nH]nc(C3CNC3)c2cn1]

To a solution of tert-butyl 3-(6-acetamido-1-(tetrahydro-2H-pyran-2-yl)-1H-pyrazolo[4,3-c]pyridin-3-yl)azetidine-1-carboxylate (Preparation 530, 455 mg, 1.10 mmol) in DCM (5 mL) was added TFA (1.5 mL) and the reaction was stirred at rt overnight. The reaction mixture was concentrated under vacuum. The residue was diluted with DCM/ether, the solid formed was filtered and washed with ether to give N-(3-(azetidin-3-yl)-1H-pyrazolo[4,3-c]pyridin-6-yl)acetamide trifluoroacetate (473 mg, 94% yield) as an off-white solid. LCMS m/z=232 [M+H]⁺

### Preparation 560: N-(3-(6-oxa-3-azabicyclo[3.1.1]heptan-3-yl)-1H-pyrazolo[4,3-c]pyridin-6-yl)formamide hydrochloride

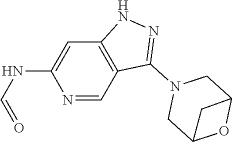 [SMILES: O=CNc1cc2[nH]nc(N3CC4CC(C3)O4)c2cn1]

To a solution of N-(3-(6-oxa-3-azabicyclo[3.1.1]heptan-3-yl)-1-(tetrahydro-2H-pyran-2-yl)-1H-pyrazolo[4,3-c]pyridin-6-yl)formamide (Preparation 523, 291 mg, 0.848 mmol) in DCM (3 mL) was added HCl/dioxane (4 M, 4 mL) and the reaction stirred at 25° C. for 2 h. The mixture was concentrated to give N-(3-(6-oxa-3-azabicyclo[3.1.1]heptan-3-yl)-1H-pyrazolo[4,3-c]pyridin-6-yl)formamide hydrochloride (220 mg, crude) as a yellow solid. LCMS m/z=260.0 [M+H]⁺

### Preparation 561: N-(3-(2-oxa-5-azabicyclo[2.2.1]heptan-5-yl)-1H-pyrazolo[4,3-c]pyridin-6-yl)acetamide hydrochloride

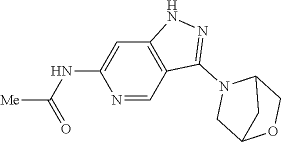 [SMILES: CC(=O)Nc1cc2[nH]nc(N3CC4CC3CO4)c2cn1]

N-(3-(2-Oxa-5-azabicyclo[2.2.1]heptan-5-yl)-1H-pyrazolo[4,3-c]pyridin-6-yl)acetamide hydrochloride was obtained as a yellow solid, 700 mg, from N-(3-(2-oxa-5-azabicyclo[2.2.1]heptan-5-yl)-1-(tetrahydro-2H-pyran-2-yl)-1H-pyrazolo[4,3-c]pyridin-6-yl)acetamide (Preparation 524), following the procedure described in Preparation 560. LCMS m/z=274.0 [M+H]⁺

### Preparation 562: N-(3-(1-(cyanomethyl)azetidin-3-yl)-1H-pyrazolo[4,3-c]pyridin-6-yl)acetamide

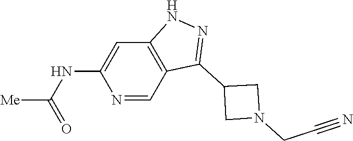 [SMILES: CC(=O)Nc1cc2[nH]nc(C3CN(CC#N)C3)c2cn1]

DIPEA (34 μL, 0.195 mmol) was added to a solution of N-(3-(azetidin-3-yl)-1H-pyrazolo[4,3-c]pyridin-6-yl)acetamide trifluoroacetate (Preparation 559, 15 mg, 64.9 μmol) and 2-bromoacetonitrile (12 mg, 97.3 μmol) in MeCN (1 mL) and the reaction was stirred at rt for 1 h. The reaction mixture was evaporated under reduced pressure to give N-(3-(1-(cyanomethyl)azetidin-3-yl)-1H-pyrazolo[4,3-c]pyridin-6-yl)acetamide. LCMS m/z=271 [M+H]⁺

### Preparation 563: N-(1-(6-bromopyridin-2-yl)-1H-pyrazolo[4,3-c]pyridin-6-yl)acetamide

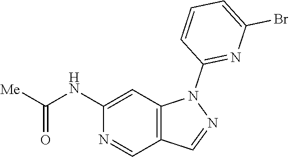

A mixture of N-(1H-pyrazolo[4,3-c]pyridin-6-yl)acetamide (100 mg, 0.568 mmol), 2,6-dibromopyridine (269 mg, 1.14 mmol) and CuI (11 mg, 0.057 mmol) in dioxane (4 mL) was purged with N₂ for 5 min and the reaction was heated at 80° C. for 4 h. The mixture was diluted with EtOAc, washed with water (3×), then brine. The organic layer was dried and concentrated. The crude was purified by chromatography on silica gel (10-100% EtOAc in heptane) to give N-(1-(6-bromopyridin-2-yl)-1H-pyrazolo[4,3-c]pyridin-6-yl)acetamide (68 mg, 36% yield) as a white powder. LCMS m/z=332, 334 [M+H]⁺

### Preparation 564: tert-butyl 3-((6-chloro-1-(tetrahydro-2H-pyran-2-yl)-1H-pyrazolo[4,3-c]pyridin-3-yl)amino)pyrrolidine-1-carboxylate

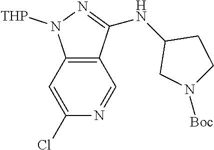

To a solution of 6-chloro-3-iodo-1-(tetrahydro-2H-pyran-2-yl)-1H-pyrazolo[4,3-c]pyridine (400 mg, 1.10 mmol) and tert-butyl 3-aminopyrrolidine-1-carboxylate (614.7 mg, 3.30 mmol) in DMSO (5 mL) was added CuI (41.9 mg, 0.22 mmol), K₂CO₃ (456.2 mg, 3.30 mmol) and L-proline (50.7 mg, 0.44 mmol) and the reaction mixture was stirred at 100° C. for 3 h under N₂. The mixture was treated with H₂ (20 mL) and extracted with EtOAc (25 mL×3). The organic phase was washed with brine (30 mL), dried over Na₂SO₄, filtered, concentrated, then purified by column chromatography (PE/EtOAc=15/1 to 1/1) on silica gel to give tert-butyl 3-((6-chloro-1-(tetrahydro-2H-pyran-2-yl)-1H-pyrazolo[4,3-c]pyridin-3-yl)amino)pyrrolidine-1-carboxylate (210 mg, 45.2% yield) as a colorless solid. ¹HNMR (500 MHz, CDCl₃) δ ppm: 8.58 (s, 1H), 7.31 (s, 1H), 5.44-5.41 (m, 1H), 4.41-4.28 (m, 2H), 4.03 (s, 1H), 3.77-3.68 (m, 2H), 3.49-3.39 (m, 3H), 2.78-2.25 (m, 3H), 1.99-1.97 (m, 1H), 1.74-1.64 (m, 3H), 1.47 (s, 9H).

### Preparations 565 to 580

The compounds in the following table were prepared from 6-chloro-3-iodo-1-(tetrahydro-2H-pyran-2-yl)-1H-pyrazolo[4,3-c]pyridine and the appropriate amine, following a similar procedure to that described in Preparation 564.

| Preparation | |
|---|---|
| No | Name, Structure, Starting Amine (SM), Data |
| 565 | 1-(6-chloro-1-(tetrahydro-2H-pyran-2-yl)-1H- |
| | pyrazolo[4,3-c]pyridin-3-yl)-5-methylpyrrolidin-3-ol |
| | 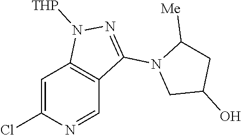 |
| | SM: 5-methylpyrrolidin-3-ol hydrochloride |
| | 189 mg, 40.8% yield as yellow oil. LCMS m/z = |
| | 337.1 [M + H]⁺ |
| 566 | 1-(6-chloro-1-(tetrahydro-2H-pyran-2-yl)-1H-pyrazolo |
| | [4,3-c]pyridin-3-yl)-4-methylpyrrolidin-3-ol |
| | 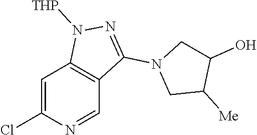 |
| | SM: 4-methylpyrrolidin-3-ol |
| | 130 mg, 46.8% yield as a white solid. LCMS m/z = |
| | 337.1 [M + H]⁺ |
| 567 | tert-butyl (1-(6-chloro-1-(tetrahydro-2H-pyran-2-yl)- |
| | 1H-pyrazolo[4,3-c]pyridin-3-yl)-4-methylpyrrolidin- |
| | 3-yl)carbamate |
| | 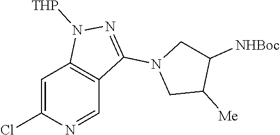 |
| | SM: tert-butyl (4-methylpyrrolidin-3-yl)carbamate |
| | 194 mg, 53.9% yield as a white solid. ¹H NMR (500 MHz, |
| | CDCl₃) δ ppm: 8.71 (d, J = 4.0 Hz, 1H), |
| | 7.31 (s, 1H), 5.41-5.39 (m, 1H), 4.70-4.64 (m, 1H,) |
| | 4.06-4.04 (m, 2H), 3.88-3.82 (m, 2H), 3.71-3.69 (m, 1H), |
| | 3.33-3.32 (m, 2H), 2.41-2.39 (m, 1H), 2.11-2.10 (m, |
| | 1H), 1.94-1.92 (m, 1H), 1.71-1.70 (m, 2H), 1.47-1.45 |
| | (m, 9H), 1.27-1.26 (m, 1H), 1.19-1.10 (m, 3 H). |
| 568 | tert-butyl (1-(6-chloro-1-(tetrahydro-2H-pyran-2-yl)- |
| | 1H-pyrazolo[4,3-c]pyridin-3-yl)-5-methylpyrrolidin-3- |
| | yl)carbamate |
| | 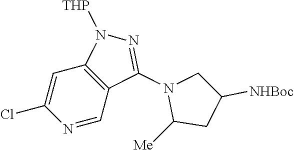 |
| | SM: tert-butyl (5-methylpyrrolidin-3-yl)carbamate |
| | 112.3 mg, 31.2% yield as a yellow solid. LCMS |
| | m/z = 436.2 [M + H]⁺ |
| 569 | (3S)-1-(6-chloro-1-(tetrahydro-2H-pyran-2-yl)-1H- |
| | pyrazolo[4,3-c]pyridin-3-yl)-N,N-dimethylpyrrolidin- |
| | 3-amine |
| | 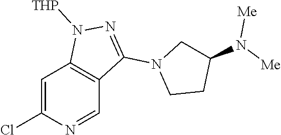 |
| | SM: (S)-N,N-dimethylpyrrolidin-3-amine |
| | 650 mg, 67.6% yield as a light-yellow solid. LCMS |
| | m/z = 350.1 [M + H]⁺ |
| 570^{A} | 6-chloro-3-(3,3-difluoro-4-methoxypyrrolidin-1- |
| | yl)-1-(tetrahydro-2H-pyran-2-yl)-1H-pyrazolo[4,3-c] |
| | pyridine |
| | 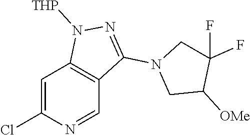 |
| | SM: 3,3-difluoro-4-methoxypyrrolidine hydrochloride |
| | 70.0 mg, crude as a white solid. LCMS m/z = 373.1 [M + H]⁺ |
| 571^{A} | 6-chloro-3-(4-methoxy-2-methylpyrrolidin-1-yl)-1- |
| | (tetrahydro-2H-pyran-2-yl)-1H-pyrazolo[4,3-c]pyridine |
| | 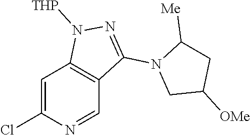 |
| | SM: 4-methoxy-2-methylpyrrolidine hydrochloride |
| | 70.0 mg, crude as a yellow gum. LCMS m/z = 351.1 |
| | [M + H]⁺ |
| 572 | Cis-rac-tert-butyl (1-(6-chloro-1-(tetrahydro-2H- |
| | pyran-2-yl)-1H-pyrazolo[4,3-c]pyridin-3-yl)-4- |
| | methylpyrrolidin-3-yl)carbamate |
| | 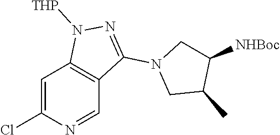 |
| | SM: cis-rac-tert-butyl (4-methylpyrrolidin-3-yl)carbamate |
| | Purified by Prep-HPLC-C (46% to 76% MeCN). |
| | 62.0 mg, 25.9% yield as a yellow solid. |
| | LCMS m/z = 436.3 [M + H]⁺ |
| 573 | 1-((6-chloro-1-(tetrahydro-2H-pyran-2-yl)-1H-pyrazolo |
| | [4,3-c]pyridin-3-yl)amino)-2-methylpropan-2-ol |
| | 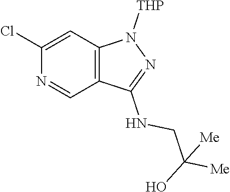 |
| | SM: 1-amino-2-methylpropan-2-ol |
| | 350 mg, 26.1% yield as a yellow solid. ¹H NMR (500 MHz, |
| | CDCl₃) δ ppm 8.67 (s, 1H), 7.30 (s, 1H), 5.41-5.38 (m, |
| | 1H), 4.07-4.02 (m, 1H), 3.73-3.66 (m, 1H), 3.51-3.46 |
| | (m, 1H), 3.42-3.37 (m, 1H), 2.41-2.31 (m, 1H), |
| | 2.13-2.04 (m, 1H), 2.02-1.94 (m, 1H), |
| | 1.77-1.66 (m, 2H), 1.65-1.59 (m, 1H), 1.33 (s, 6H) |
| 574 | 6-chloro-N-(2-methoxyethyl)-N-methyl-1-(tetrahydro-2H- |
| | pyran-2-yl)-1H-pyrazolo[4,3-c]pyridin-3-amine |
| | 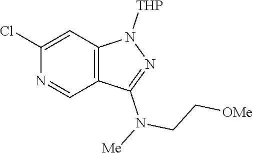 |
| | SM: 2-methoxy-N-methylethan-1-amine |
| | 147 mg, 32.9% yield as white solid. ¹H NMR (400 MHz, |
| | CDCl₃) δ ppm: 8.83 (s, 1H), 7.29 (s, 1H), 5.43-5.40 |
| | (m, 1H), 3.72-3.65 (m, 6H), 3.37 (s, 3H), 3.21 (s, 3H), 2.14- |
| | 2.05 (m, 1H), 1.99-1.91 (m, 1H), 1.67-1.61 (m, 4H) |
| 575 | N-benzyl-6-chloro-N-methyl-1-(tetrahydro-2H-pyran- |
| | 2-yl)-1H-pyrazolo[4,3-c]pyridin-3-amine |
| | 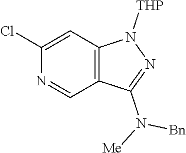 |
| | SM: N-methylbenzylamine |
| | 220 mg, 22.4% yield as a white solid. LCMS m/z = 357.2 |
| | [M + H]⁺ |
| 576 | N-benzyl-6-chloro-1-(tetrahydro-2H-pyran-2-yl)-1H- |
| | pyrazolo[4,3-c]pyridin-3-amine |
| | 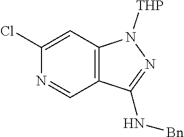 |
| | SM: benzylamine |
| | 1.2 g, 65.2% yield as a yellow solid. LCMS m/z = 343.1 |
| | [M + H]⁺ |
| 577 | 1-(6-chloro-1-(tetrahydro-2H-pyran-2-yl)-1H-pyrazolo |
| | [4,3-c]pyridin-3-yl)-N,N,3-trimethylazetidin-3-amine |
| | 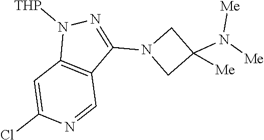 |
| | SM: N,N,3-trimethylazetidin-3-amine |
| | Prep-HPLC-D (18% to 38% MeCN) |
| | 250 mg, 52.0% yield as a yellow solid. LCMS m/z = 350.1 |
| | [M + H]⁺ |
| 580 | tert-butyl 2-(6-chloro-1-(tetrahydro-2H-pyran-2-yl)-1H- |
| | pyrazolo[4,3-c]pyridin-3-yl)-2,7-diazaspiro[3.5] |
| | nonane-7-carboxylate |
| | 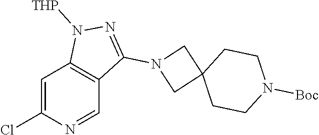 |
| | SM: tert-butyl 2,7-diazaspiro[3.5]nonane-7-carboxylate |
| | 342 mg, 53.8% as yellow gum |
| | ¹H NMR (500 MHz, CDCl₃) δ ppm: 8.55 (s, 1H), |
| | 7.30 (s, 1H), 5.40 (d, J = 9.5 Hz, 1H), 4.10-4.06 (m, 1H), 3.98- |
| | 3.93 (m, 4H), 3.72-3.68 (m, 1H), 3.41 (s, 4H), 2.40-2.38 |
| | (m, 1H), 2.10-1.98 (m, 1H), 1.95-1.82 (m, 1H), 1.82 (s, 4H), |
| | 1.72-1.68 (m, 2H), 1.63-1.46 (m, 1H), 1.46 (m, 9H) |

A-DMF was used as the reaction solvent

### Preparation 581: tert-butyl 3-(6-chloro-1-(tetrahydro-2H-pyran-2-yl)-1H-pyrazolo[4,3-c]pyridin-3-yl)-3,9-diazabicyclo[3.3.1]nonane-9-carboxylate

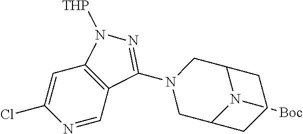

To a solution of 6-chloro-3-iodo-1-(tetrahydro-2H-pyran-2-yl)-1H-pyrazolo[4,3-c]pyridine (500 mg, 1.38 mmol) in toluene (10 mL) was added tert-butyl 3,9-diazabicyclo[3.3.1]nonane-9-carboxylate (373.5 mg, 1.65 mmol), Cphos Pd G₃ (110.9 mg, 0.14 mmol) and t-BuONa (396.5 mg, 4.13 mmol) and the reaction mixture was stirred at 110° C. for 16 h under N₂. The mixture was concentrated and the residue was purified by chromatography on silica gel (PE/EtOAc=1/1) to give tert-butyl 3-(6-chloro-1-(tetrahydro-2H-pyran-2-yl)-1H-pyrazolo[4,3-c]pyridin-3-yl)-3,9-diazabicyclo[3.3.1]nonane-9-carboxylate (280 mg, 44.1% yield) as a yellow oil. ¹H NMR (400 MHz, CDCl₃) δ: ppm 8.79 (s, 1H), 7.34 (s, 1H), 5.45-5.20 (m, 1H), 4.06-4.04 (m, 1H), 3.90-3.88 (m, 2H), 3.73-3.71 (m, 1H), 3.38-3.34 (m, 2H), 2.47-2.45 (m, 2H), 2.11-2.10 (m, 2H), 1.97-1.62 (m, 10H), 1.51-1.46 (m, 9H).

### Preparation 582: tert-butyl ((3R)-1-(6-chloro-1-(tetrahydro-2H-pyran-2-yl)-1H-pyrazolo[4,3-c]pyridin-3-yl)pyrrolidin-3-yl)carbamate

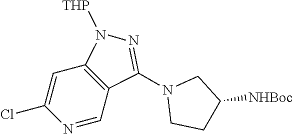

To a solution of 6-chloro-3-iodo-1-(tetrahydro-2H-pyran-2-yl)-1H-pyrazolo[4,3-c]pyridine (3 g, 8.25 mmol) in toluene (50 mL) was added tert-butyl (R)-pyrrolidin-3-ylcarbamate (3.1 g, 16.5 mmol), Cs₂CO₃ (5.4 g, 16.5 mmol) and Ruphos Pd G3 (690.1 mg, 0.825 mmol) and the reaction was stirred at 110° C. for 16 h under N₂. The mixture was concentrated and the residue was purified by chromatography on silica gel (PE/EtOAc=3/1) to give tert-butyl ((3R)-1-(6-chloro-1-(tetrahydro-2H-pyran-2-yl)-1H-pyrazolo[4,3-c]pyridin-3-yl)pyrrolidin-3-yl)carbamate (1.2 g, 34.5% yield) as a yellow solid. LCMS m/z=422.1 [M+H]⁺

### Preparations 583 to 587

The compounds in the following table were prepared from 6-chloro-3-iodo-1-(tetrahydro-2H-pyran-2-yl)-1H-pyrazolo[4,3-c]pyridine and the appropriate amine, following a similar procedure to that described in Preparation 582.

| Preparation | |
|---|---|
| No. | Name, Structure, Starting Material (SM), Data |
| 583 | tert-butyl (1-(6-chloro-1-(tetrahydro-2H-pyran-2- |
| | yl)-1H-pyrazolo[4,3-c]pyridin-3-yl)azetidin-3-yl) |
| | carbamate |
| | 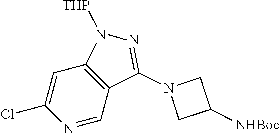 |
| | SM: tert-butyl azetidin-3-ylcarbamate |
| | 800 mg, 35.7% yield as a yellow solid. ¹H NMR: (400 MHz, |
| | CDCl₃) δ: ppm 8.54 (s, 1H), 7.32 (s, 1H), 5.43-5.40 (m, |
| | 1H), 5.00 (br s, 1H), 4.72 (br s, 1H), 4.54-4.49 (m, 2H), |
| | 4.10-4.03 (m, 1H), 4.01-3.94 (m, 2H), 3.73-3.70 |
| | (m, 1H), 2.43-2.35 (m, 1H), 2.10 (br s, 1H), 1.98- |
| | 1.94 (m, 1H), 1.73-1.68 (m, 3H), 1.46 |
| | (s, 9H). |
| 584 | tert-butyl (1-(6-chloro-1-(tetrahydro-2H-pyran-2-yl)-1H- |
| | pyrazolo[4,3-c]pyridin-3-yl)-4,4-difluoropyrrolidin- |
| | 3-yl)carbamate |
| | 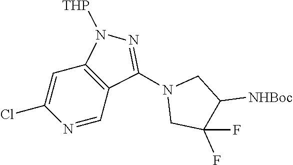 |
| | SM: tert-butyl (4,4-difluoropyrrolidin-3-yl)carbamate |
| | 250 mg, 80.9% yield as a yellow solid. ¹H NMR (400 |
| | MHz, CDCl₃) δ ppm 8.69 (d, J = 3.2 Hz, 1H), |
| | 7.33 (s, 1H), 5.45-5.41 (m, 1H), 4.97-4.95 (m, 1H), |
| | 4.69-4.65 (m, 1H), 4.22-4.20 (m, 1H), 4.08-3.99 (m, 3H), |
| | 3.73-3.70 (m, 1H), 3.52-3.50 (m, 1H), 2.43- |
| | 2.41 (m, 1H), 2.11-2.10 (m, 1H), 1.98-1.95 (m, 1H), |
| | 1.74-1.63 (m, 3H), 1.47 (s, 9H). |
| 585 | 1-(6-chloro-1-(tetrahydro-2H-pyran-2-yl)-1H-pyrazolo |
| | [4,3-c]pyridin-3-yl)-3-(difluoromethyl)pyrrolidin-3-ol |
| | 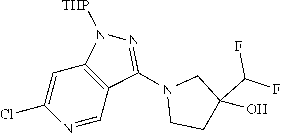 |
| | SM: 3-(difluoromethyl)pyrrolidin-3-ol |
| | 285.6 mg, 55.7% yield, as yellow solid. ¹H NMR: (400 |
| | MHz, CDCl₃) δ ppm: 8.73 (s, 1H), 7.31 (s, 1H), 5.89 |
| | (d, J = 56.0 Hz, 1H), 5.44-5.40 (m, 1H), 4.04-3.93 (m, |
| | 1H), 3.91-3.88 (m, 3H), 3.71-3.67 (m, 2H), 2.41-2.32 |
| | (m, 2H), 2.12-2.11 (m, 2H), 1.98-1.95 (m, 1H), 1.74- |
| | 1.62 (m, 4H). |
| 586 | 1-(6-chloro-1-(tetrahydro-2H-pyran-2-yl)-1H-pyrazolo- |
| | [4,3-c]pyridin-3-yl)-N,N-dimethylpiperidin-4-amine |
| | 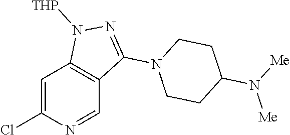 |
| | SM: N,N-dimethylpiperidin-4-amine |
| | 260 mg, 52.0% yield as a yellow solid. ¹H NMR: (400 MHz, |
| | CDCl₃) δ: ppm 8.76 (s, 1H), 7.34 (s, 1H), 5.46-5.43 |
| | (m, 1H), 4.09-4.02 (m, 3H), 3.73-3.67 (m, 1H), 3.03-2.97 |
| | (m, 2H), 2.46 (s, 6H), 2.42-2.40 (m, 1H), 2.09-2.06 (m, 3H), |
| | 2.02-1.99 (m, 1H), 1.81-1.70 (m, 6H). |
| 587 | tert-butyl 2-(6-chloro-1-(tetrahydro-2H-pyran-2-yl)-1H- |
| | pyrazolo[4,3-c]pyridin-3-yl)-2,6-diazaspiro[3.4]octane- |
| | 6-carboxylate |
| | 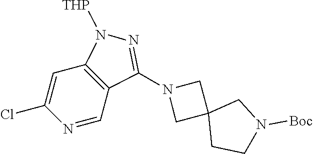 |
| | SM: tert-butyl 2,6-diazaspiro[3.4]octane-6-carboxylate |
| | 250 mg, 67.6% yield as a yellow solid. LCMS m/z = 448.3 |
| | [M + H]⁺ |

### Preparation 588: tert-butyl 7-(6-chloro-1-(tetrahydro-2H-pyran-2-yl)-1H-pyrazolo[4,3-c]pyridin-3-yl)-2,7-diazaspiro[4.4]nonane-2-carboxylate

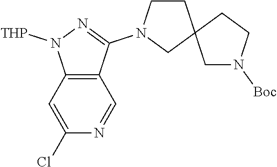

To a solution of 6-chloro-3-iodo-1-(tetrahydro-2H-pyran-2-yl)-1H-pyrazolo[4,3-c]pyridine (1.1 g, 2.94 mmol) and tert-butyl 2,7-diazaspiro[4.4]nonane-2-carboxylate (999 mg, 4.41 mmol) in dioxane (3 mL) was added Cs₂CO₃ (2.9 g, 8.83 mmol) and Xantphos Pd G₃ (304.1 mg, 0.294 mmol) and the reaction was stirred at 110° C. for 16 h under N₂. The reaction mixture was purified by prep-HPLC-A (48% to 78% MeCN) to give tert-butyl 7-(6-chloro-1-(tetrahydro-2H-pyran-2-yl)-1H-pyrazolo[4,3-c]pyridin-3-yl)-2,7-diazaspiro[4.4]nonane-2-carboxylate (460 mg, 33.8% yield) as a brown solid. LCMS m/z=462.2 [M+H]⁺

### Preparation 589: 1-(6-chloro-1-(tetrahydro-2H-pyran-2-yl)-1H-pyrazolo[4,3-c]pyridin-3-yl)-N-(4-methoxybenzyl)-N,4-dimethylpyrrolidin-3-amine

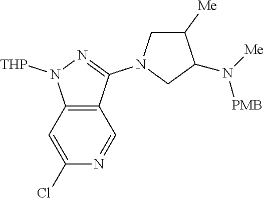

To a solution of 6-chloro-3-iodo-1-(tetrahydro-2H-pyran-2-yl)-1H-pyrazolo[4,3-c]pyridine (2.0 g, 5.50 mmol) and N-(4-methoxybenzyl)-N,4-dimethylpyrrolidin-3-amine hydrochloride (Preparation 492, 1.5 g, 5.50 mmol) in dioxane (40 mL) was added Cs₂CO₃ (5.4 g, 16.50 mmol) and XantPhos Pd G₃ (568.4 mg, 0.55 mmol) and the reaction was stirred at 100° C. for 12 h under N₂. The mixture was quenched with H₂O (50 mL) and extracted with EtOAc (30 mL×3). The combined organic layers were washed with H₂O (30 mL×3) and brine (20 mL×3), dried over Na₂SO₄, filtered and concentrated under vacuum. The crude was purified by column chromatography on silica gel (PE/EtOAc=1/0 to 1/1) to give 1-(6-chloro-1-(tetrahydro-2H-pyran-2-yl)-1H-pyrazolo[4,3-c]pyridin-3-yl)-N-(4-methoxybenzyl)-N,4-dimethylpyrrolidin-3-amine (503.0 mg, 19.5% yield) as a yellow solid. ¹HNMR (400 MHz, CDCl₃) δ ppm: 8.71 (s, 1H) 7.28-7.27 (m, 3H), 6.89-6.86 (m, 2H), 5.42-5.39 (m, 1H), 4.13-4.09 (m, 1H), 3.86-3.79 (m, 5H), 3.69-3.57 (m, 4H), 2.96-2.94 (m, 1H), 2.60-2.59 (m, 1H), 2.51-2.37 (m, 1H), 2.11 (s, 3H), 2.04 (s, 1H) 1.98-1.96 (m, 1H), 1.76-1.18 (m, 4H), 1.18-1.16 (m, 3H)

### Preparation 590: benzyl (1-(6-chloro-1-(tetrahydro-2H-pyran-2-yl)-1H-pyrazolo[4,3-c]pyridin-3-yl)pyrrolidin-3-yl)(methyl)carbamate

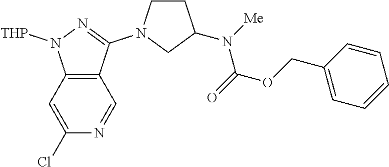

Benzyl (1-(6-chloro-1-(tetrahydro-2H-pyran-2-yl)-1H-pyrazolo[4,3-c]pyridin-3-yl)pyrrolidin-3-yl)(methyl)carbamate was obtained as a white solid, 422 mg, 54.4%, from benzyl N-methyl-N-(pyrrolidin-3-yl)carbamate and 6-chloro-3-iodo-1-(tetrahydro-2H-pyran-2-yl)-1H-pyrazolo[4,3-c]pyridine, following a similar procedure to that described in Preparation 589. ¹H NMR (400 MHz, CDCl₃) δ ppm: 8.72 (s, 1H), 7.38-7.33 (m, 5H), 7.29 (s, 1H), 5.44-5.40 (m, 1H), 5.17 (s, 2H), 4.07-4.04 (m, 1H), 3.83-3.55 (m, 5H), 2.93 (s, 3H), 2.18-2.13 (m, 5H), 2.11-2.10 (m, 1H), 1.74-1.61 (m, 3H).

### Preparation 591: tert-butyl (1-(6-chloro-1-(tetrahydro-2H-pyran-2-yl)-1H-pyrazolo[4,3-c]pyridin-3-yl)piperidin-3-yl)carbamate

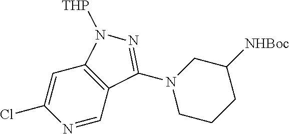

To a solution of 6-chloro-3-iodo-1-(tetrahydro-2H-pyran-2-yl)-1H-pyrazolo[4,3-c]pyridine (500 mg, 1.38 mmol) in dioxane (10 mL) was added tert-butyl piperidin-3-ylcarbamate (550.8 mg, 2.75 mmol), Pd₂(dba)₃ (125.9 mg, 0.138 mmol), Xantphos (79.6 mg, 0.138 mmol) and Cs₂CO₃ (1.3 g, 4.13 mmol) and the mixture was stirred at 100° C. for 12 h under N₂. The mixture was treated with H₂O (20 mL) and extracted with EtOAc (15 mL×3). The organic phase was washed with brine (20 mL), dried with Na₂SO₄, filtered and concentrated. The residue was purified by column chromatography (PE/EtOAc=15/1 to 1/1) on silica gel and then further purified by Prep-TLC (PE/EtOAc=1/1) to give tert-butyl (1-(6-chloro-1-(tetrahydro-2H-pyran-2-yl)-1H-pyrazolo[4,3-c]pyridin-3-yl)piperidin-3-yl)carbamate (150 mg, 25.0% yield) as a yellow solid. LCMS m/z=436.2 [M+H]⁺

### Preparation 592: tert-butyl 3-(6-chloro-1-(tetrahydro-2H-pyran-2-yl)-1H-pyrazolo[4,3-c]pyridin-3-yl)-3,6-diazabicyclo[3.1.1]heptane-6-carboxylate

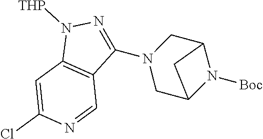

tert-Butyl 3-(6-chloro-1-(tetrahydro-2H-pyran-2-yl)-1H-pyrazolo[4,3-c]pyridin-3-yl)-3, 6-diazabicyclo[3.1.1]heptane-6-carboxylate was obtained as a yellow solid, 2.1 g, 74.4%, from 6-chloro-3-iodo-1-(tetrahydro-2H-pyran-2-yl)-1H-pyrazolo[4,3-c]pyridine and tert-butyl 3,6-diazabicyclo[3.1.1]heptane-6-carboxylate, following a similar procedure to that described in Preparation 591. LCMS m/z=434.1 [M+H]⁺

### Preparation 593: tert-butyl 9-(6-chloro-1-(tetrahydro-2H-pyran-2-yl)-1H-pyrazolo[4,3-c]pyridin-3-yl)-6-oxa-2,9-diazaspiro[4.5]decane-2-carboxylate

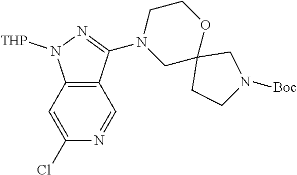

To a solution of 6-chloro-3-iodo-1-(tetrahydro-2H-pyran-2-yl)-1H-pyrazolo[4,3-c]pyridine (500 mg, 1.38 mmol) in dioxane (10 mL) was added tert-butyl 6-oxa-2,9-diazaspiro[4.5]decane-2-carboxylate (333.2 mg, 1.38 mmol), Pd₂(dba)₃ (251.9 mg, 0.275 mmol), Xantphos (238.7 mg, 0.413 mmol) and Cs₂CO₃ (896.1 mg, 2.75 mmol) and the reaction was stirred at 100° C. for 16 h under N₂. The mixture was concentrated and the residue was purified by chromatography on silica gel (PE/EtOAc=1/1) to give tert-butyl 9-(6-chloro-1-(tetrahydro-2H-pyran-2-yl)-1H-pyrazolo[4,3-c]pyridin-3-yl)-6-oxa-2,9-diazaspiro[4.5]decane-2-carboxylate (250 mg, 38.0% yield) as a yellow solid. LCMS m/z=478.3 [M+H]⁺

### Preparation 594: tert-butyl 3-((6-chloro-1-(tetrahydro-2H-pyran-2-yl)-1H-pyrazolo[4,3-c]pyridin-3-yl)(methyl)amino)pyrrolidine-1-carboxylate

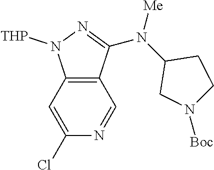

To a solution of tert-butyl 3-((6-chloro-1-(tetrahydro-2H-pyran-2-yl)-1H-pyrazolo[4,3-c]pyridin-3-yl)amino)pyrrolidine-1-carboxylate (Preparation 564, 200 mg, 0.474 mmol) in THF (5 mL) was added NaH (22.75 mg, 0.57 mmol, 60% purity), the mixture stirred at 25° C. for 30 mins, then CH₃I (134.6 mg, 0.948 mmol) was added. The reaction mixture was stirred at 25° C. for 12 h. The mixture was concentrated, treated with H₂O (20 mL) and extracted with EtOAc (15 mL×3). The organic phase was washed with brine (40 mL), dried with Na₂SO₄, filtered, concentrated, then purified by column chromatography (PE/EtOAc=15/1 to 1/1) on silica gel to give tert-butyl 3-((6-chloro-1-(tetrahydro-2H-pyran-2-yl)-1H-pyrazolo[4,3-c]pyridin-3-yl)(methyl)amino)pyrrolidine-1-carboxylate (150 mg, 72.6% yield) as a colorless solid. ¹H NMR: (400 MHz, CDCl₃) δ ppm: 8.78 (s, 1H), 7.33-7.31 (m, 1H), 5.45-5.41 (m, 1H), 4.70-4.67 (m, 1H), 4.06-4.02 (m, 1H), 3.73-3.61 (m, 5H), 3.37 (s, 3H), 2.43-2.41 (m, 1H), 2.20-2.15 (m, 3H), 2.10-1.95 (m, 1H), 1.72-1.62 (m, 3H), 1.47 (s, 9H).

### Preparation 595: 6-chloro-N-(2-methoxy-2-methylpropyl)-N-methyl-1-(tetrahydro-2H-pyran-2-yl)-1H-pyrazolo[4,3-c]pyridin-3-amine

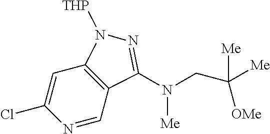

6-Chloro-N-(2-methoxy-2-methylpropyl)-N-methyl-1-(tetrahydro-2H-pyran-2-yl)-1H-pyrazolo[4,3-c]pyridin-3-amine was obtained as a yellow solid, 300 mg, 92.1% yield, from 1-((6-chloro-1-(tetrahydro-2H-pyran-2-yl)-1H-pyrazolo[4,3-c]pyridin-3-yl)amino)-2-methylpropan-2-ol (Preparation 573) and CH₃I following a similar procedure to that described in Preparation 594. ¹H NMR (400 MHz, CDCl₃) δ ppm 8.88 (s, 1H), 7.27 (s, 1H), 5.42-5.36 (m, 1H), 4.02 (d, J=10.8 Hz, 1H), 3.73-3.64 (m, 2H), 3.50-3.43 (m, 1H), 3.29 (s, 3H), 3.25 (s, 3H), 2.49-2.35 (m, 1H), 2.15-2.06 (m, 1H), 1.99-1.89 (m, 1H), 1.77-1.62 (m, 3H), 1.21 (s, 6H).

### Preparation 596: tert-butyl (1-(6-chloro-1-(tetrahydro-2H-pyran-2-yl)-1H-pyrazolo[4,3-c]pyridin-3-yl)-4,4-difluoropyrrolidin-3-yl)(methyl)carbamate

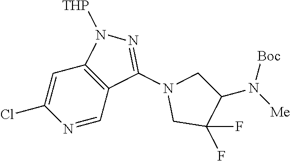

tert-Butyl (1-(6-chloro-1-(tetrahydro-2H-pyran-2-yl)-1H-pyrazolo[4,3-c]pyridin-3-yl)-4,4-difluoropyrrolidin-3-yl)(methyl)carbamate was obtained as a yellow solid, 210 mg, 92.6%, from tert-butyl (1-(6-chloro-1-(tetrahydro-2H-pyran-2-yl)-1H-pyrazolo[4,3-c]pyridin-3-yl)-4,4-difluoropyrrolidin-3-yl)carbamate (Preparation 584) and MeI, following the procedure described in Preparation 594. ¹H NMR (400 MHz, CDCl₃) δ: ppm 8.72 (s, 1H), 7.33 (s, 1H), 5.46-5.43 (m, 1H), 4.03-3.87 (m, 5H), 3.74-3.71 (m, 1H), 2.95-2.94 (m, 3H), 2.43-2.40 (m, 1H), 2.10-2.09 (m, 1H), 2.01-1.99 (m, 1H), 1.75-1.63 (m, 4H), 1.50 (s, 9H).

### Preparation 597: N-benzyl-6-chloro-N-ethyl-1-(tetrahydro-2H-pyran-2-yl)-1H-pyrazolo[4,3-c]pyridin-3-amine

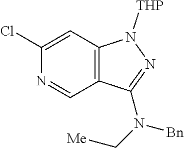

N-Benzyl-6-chloro-N-ethyl-1-(tetrahydro-2H-pyran-2-yl)-1H-pyrazolo[4,3-c]pyridin-3-amine was obtained as yellow soil, 500 mg, 77% from N-benzyl-6-chloro-1-(tetrahydro-2H-pyran-2-yl)-1H-pyrazolo[4,3-c]pyridin-3-amine (Preparation 576) and ethyl iodide following a similar procedure to that described in Preparation 594. LCMS m/z=371.1 [M+H]⁺

### Preparation 598: 6-chloro-3-(3-methoxy-4-methylpyrrolidin-1-yl)-1-(tetrahydro-2H-pyran-2-yl)-1H-pyrazolo[4,3-c]pyridine

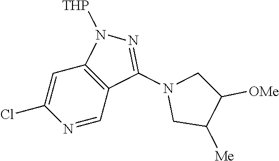

A solution of 1-(6-chloro-1-(tetrahydro-2H-pyran-2-yl)-1H-pyrazolo[4,3-c]pyridin-3-yl)-4-methylpyrrolidin-3-ol (Preparation 566, 150 mg, 0.445 mmol) and NaH (21.4 mg, 0.534 mmol, 60% purity) in DMF (4 mL) was stirred at 0° C. for 10 min. MeI (69.6 mg, 0.49 mmol) was added and the reaction stirred for 2 h at 25° C. The mixture was quenched with H₂O (50 mL) and extracted with EtOAc (20 mL×3). The combined organic layers were washed with H₂O (20 mL×3) and brine (20 mL), dried over Na₂SO₄, filtered and concentrated under vacuum. The crude product was purified on silica gel column chromatography (PE/EtOAc=1/0 to 3/1) to give 6-chloro-3-(3-methoxy-4-methylpyrrolidin-1-yl)-1-(tetrahydro-2H-pyran-2-yl)-1H-pyrazolo[4,3-c]pyridine (130 mg, 83.2% yield) as a white solid. LCMS m/z=351.2 [M+H]⁺

### Preparation 599: 6-chloro-3-(3,3-difluorocyclobutyl)-1-(tetrahydro-2H-pyran-2-yl)-1H-pyrazolo[4,3-c]pyridine

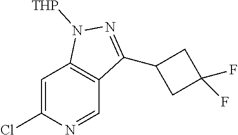

A mixture of NiCl₂ glyme (1.61 mg, 7.31 μmol) and 4-tert-butyl-2-(4-tert-butyl-2-pyridyl)pyridine (1.96 mg, 7.31 μmol) in dioxane (4 mL) was sonicated and stirred and the solution was sparged with N₂ for 10 min. The Ni solution was added to a mixture of 3-bromo-1,1-difluoro-cyclobutane (500 mg, 2.92 mmol), 3-bromo-6-chloro-1-tetrahydropyran-2-yl-pyrazolo[4,3-c]pyridine (462.9 mg, 1.46 mmol), Ir[dF(CF₃)ppy]₂(dtbpy)PF₆ (16.4 mg, 14.6 μmol) and 2,6-lutidine (783.3 mg, 7.31 mmol) in dioxane (14 mL). Bis(trimethylsilyl)silyl-trimethyl-silane (1.09 g, 4.39 mmol) was added, the reaction was degassed for 15 min and then irradiated with 2×450 nM kessile lamps with cooling. After 2 h, the solvent was removed, the residue was diluted with DCM and washed with NaHCO₃ (sat). The organic layer was solid loaded on to silica gel and purified by silica gel chromatography (heptane to EtOAc) to give 6-chloro-3-(3,3-difluorocyclobutyl)-1-(tetrahydro-2H-pyran-2-yl)-1H-pyrazolo[4,3-c]pyridine, 174 mg. LCMS m/z=328 [M+H]⁺

### Preparation 600: 6-chloro-N-methyl-N-(pyrrolidin-3-yl)-1H-pyrazolo[4,3-c]pyridin-3-amine hydrochloride

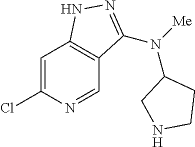 [SMILES: CN(c1n[nH]c2cc(Cl)ncc12)C1CCNC1]

To a solution of tert-butyl 3-((6-chloro-1-(tetrahydro-2H-pyran-2-yl)-1H-pyrazolo[4,3-c]pyridin-3-yl)(methyl)amino)pyrrolidine-1-carboxylate (Preparation 594, 161.0 mg, 0.37 mmol) in DCM (5 mL) was added HCl/dioxane (4 M, 5 mL) and the reaction was stirred at 25° C. for 2 h. The mixture was concentrated to give 6-chloro-N-methyl-N-(pyrrolidin-3-yl)-1H-pyrazolo[4,3-c]pyridin-3-amine hydrochloride (100 mg, crude) as a yellow solid. LCMS m/z=252 [M+H]⁺

### Preparation 601 to 621

The compounds in the following table were prepared from the appropriate protected amine, following a similar procedure to that described in Preparation 600.

| Preparation | |
|---|---|
| No | Name, Structure, Starting Material (SM), Yield, Data |
| 601 | 3-(3,9-diazabicyclo[3.3.1]nonan-3-yl)-6-chloro- |
| | 1H-pyrazolo[4,3-c]pyridine hydrochloride |
| | 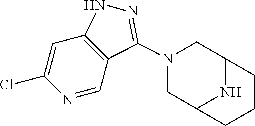 [SMILES: Clc1cc2[nH]nc(N3CC4CCCC(C3)N4)c2cn1] |
| | SM: tert-butyl 3-(6-chloro-1-(tetrahydro- |
| | 2H-pyran-2-yl)-1H-pyrazolo[4,3- |
| | c]pyridin-3-yl)-3,9-diazabicyclo[3.3.1]nonane-9-carboxylate |
| | (Preparation 581) |
| | Yellow solid, 190 mg, crude. LCMS m/z = 278.1 [M + H]⁺ |
| 602 | (R)-1-(6-chloro-1H-pyrazolo[4,3-c]pyridin-3-yl)pyrrolidin- |
| | 3-amine hydrochloride |
| | 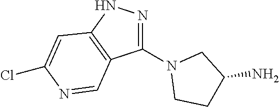 [SMILES: N[C@@H]1CCN(c2n[nH]c3cc(Cl)ncc23)C1] |
| | SM: tert-butyl ((3R)-1-(6-chloro-1-(tetrahydro-2H- |
| | pyran-2-yl)-1H-pyrazolo[4,3-c]pyridin-3-yl) |
| | pyrrolidin-3-yl)carbamate (Preparation 582) |
| | 670 mg, 99% as a yellow solid. |
| | LCMS m/z = 238.0 [M + H]⁺ |
| 603 | 1-(6-chloro-1H-pyrazolo[4,3-c]pyridin-3-yl)-4- |
| | methylpyrrolidin-3-ol hydrochloride |
| | 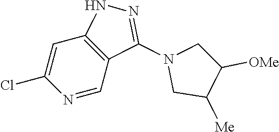 [SMILES: COC1CN(c2n[nH]c3cc(Cl)ncc23)CC1C] |
| | SM: 1-(6-chloro-1-(tetrahydro-2H-pyran-2-yl)-1H- |
| | pyrazolo[4,3-c]pyridin-3-yl)-4- |
| | methylpyrrolidin-3-ol (Preparation 566) |
| | 70 mg, 74.1% as yellow solid. |
| | LCMS m/z = 253.0 [M + H]⁺ |
| 604 | 6-chloro-3-(3-methoxy-4-methylpyrrolidin-1-yl)- |
| | 1H-pyrazolo[4,3-c]pyridine hydrochloride |
| | 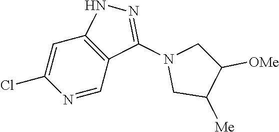 [SMILES: COC1CN(c2n[nH]c3cc(Cl)ncc23)CC1C] |
| | SM: 6-chloro-3-(3-methoxy-4-methylpyrrolidin-1-yl)- |
| | 1-(tetrahydro-2H-pyran-2-yl)-1H- |
| | pyrazolo[4,3-c]pyridine (Preparation 598) |
| | 70 mg, as a yellow solid. |
| | LCMS m/z = 267.1 [M + H]⁺ |
| 605 | 6-chloro-3-(4-methoxy-2-methylpyrrolidin-1-yl)- |
| | 1H-pyrazolo[4,3-c]pyridine hydrochloride |
| | 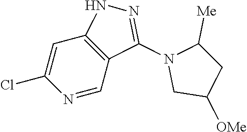 [SMILES: COC1CC(C)N(c2n[nH]c3cc(Cl)ncc23)C1] |
| | SM: 6-chloro-3-(4-methoxy-2-methylpyrrolidin-1-yl)- |
| | 1-(tetrahydro-2H-pyran-2-yl)-1H-pyrazolo |
| | [4,3-c]pyridine (Preparation 571) |
| | 40.0 mg, as a yellow solid. LCMS m/z = 267.1 [M + H]⁺ |
| 606 | 1-(6-chloro-1H-pyrazolo[4,3-c]pyridin-3-yl)-5- |
| | methylpyrrolidin-3-amine hydrochloride |
| | 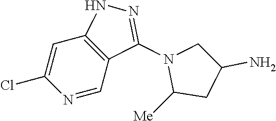 [SMILES: CC1CC(N)CN1c1n[nH]c2cc(Cl)ncc12] |
| | SM: tert-butyl (1-(6-chloro-1-(tetrahydro-2H-pyran-2- |
| | yl)-1H-pyrazolo[4,3-c]pyridin-3-yl)-5- |
| | methylpyrrolidin-3-yl)carbamate (Preparation 568) |
| | 90.0 mg, crude, as a yellow solid. |
| | LCMS m/z = 252.1 [M + H]⁺ |
| 607 | 1-(6-chloro-1H-pyrazolo[4,3-c]pyridin-3-yl)-4,4-difluoro- |
| | N-methylpyrrolidin-3-amine hydrochloride |
| | 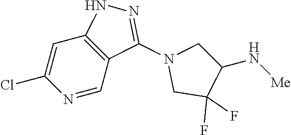 [SMILES: CNC1CN(c2n[nH]c3cc(Cl)ncc23)CC1(F)F] |
| | SM: tert-butyl (1-(6-chloro-1-(tetrahydro-2H-pyran- |
| | 2-yl)-1H-pyrazolo[4,3-c]pyridin-3-yl)-4,4- |
| | difluoropyrrolidin-3-yl)(methyl)carbamate |
| | (Preparation 596) |
| | 170 mg, crude as a yellow solid. |
| | LCMS m/z = 288.0 [M + H]⁺ |
| 608 | Cis-rac-1-(6-chloro-1H-pyrazolo[4,3-c]pyridin-3-yl)-4- |
| | methylpyrrolidin-3-amine hydrochloride |
| | 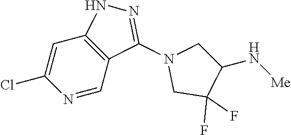 [SMILES: CNC1CN(c2n[nH]c3cc(Cl)ncc23)CC1(F)F] |
| | SM: cis-rac-tert-butyl (1-(6-chloro-1-(tetrahydro- |
| | 2H-pyran-2-yl)-1H-pyrazolo[4,3-c] |
| | pyridin-3-yl)-4-methylpyrrolidin-3-yl)carbamate |
| | (Preparation 572) |
| | 80 mg, as a yellow solid. LCMS m/z = 252.2 [M + H]⁺ |
| 609 | 1-(6-chloro-1H-pyrazolo[4,3-c]pyridin-3-yl)-4,4- |
| | difluoropyrrolidin-3-amine hydrochloride |
| | 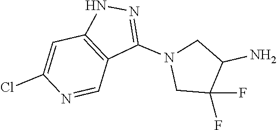 [SMILES: NC1CN(c2n[nH]c3cc(Cl)ncc23)CC1(F)F] |
| | SM: tert-butyl (1-(6-chloro-1-(tetrahydro-2H-pyran- |
| | 2-yl)-1H-pyrazolo[4,3-c]pyridin-3-yl)-4,4- |
| | difluoropyrrolidin-3-yl)carbamate (Preparation 584) |
| | 130 mg, crude, as a yellow solid. |
| | LCMS m/z = 274.1 [M + H]⁺ |
| 610 | 1-(6-chloro-1H-pyrazolo[4,3-c]pyridin-3-yl)-3- |
| | (difluoromethyl)pyrrolidin-3-ol hydrochloride |
| | 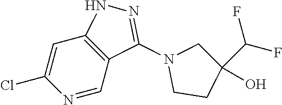 [SMILES: OC1(C(F)F)CCN(c2n[nH]c3cc(Cl)ncc23)C1] |
| | SM: 1-(6-chloro-1-(tetrahydro-2H-pyran-2-yl)-1H- |
| | pyrazolo[4,3-c]pyridin-3-yl)-3-(difluoromethyl) |
| | pyrrolidin-3-ol (Preparation 585) |
| | 150 mg, 68.6% as yellow solid. |
| | LCMS m/z = 289.1 [M + H]⁺ |
| 611 | 6-chloro-N-(2-methoxy-2-methylpropyl)- |
| | N-methyl-1H-pyrazolo[4,3- |
| | c]pyridin-3-amine hydrochloride |
| | 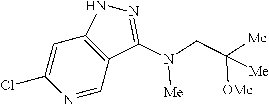 [SMILES: COC(C)(C)CN(C)c1n[nH]c2cc(Cl)ncc12] |
| | SM: 6-chloro-N-(2-methoxy-2-methylpropyl)- |
| | N-methyl-1-(tetrahydro-2H-pyran-2-yl)- |
| | 1H-pyrazolo[4,3-c]pyridin-3-amine (Preparation 595) |
| | 280 mg as a white solid. LCMS m/z = 269.2 [M + H]⁺ |
| 612 | 6-chloro-N-(2-methoxyethyl)-N-methyl-1H-pyrazolo |
| | [4,3-c]pyridin-3-amine hydrochloride |
| | 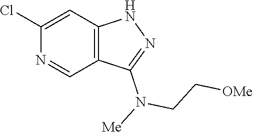 [SMILES: COCCN(C)c1n[nH]c2cc(Cl)ncc12] |
| | SM: 6-chloro-N-(2-methoxyethyl)-N-methyl-1- |
| | (tetrahydro-2H-pyran-2-yl)-1H-pyrazolo |
| | [4,3-c]pyridin-3-amine (Preparation 574) |
| | 120 mg, white solid. LCMS m/z = 241.1 [M + H]⁺ |
| 613 | N-benzyl-6-chloro-N-methyl-1H-pyrazolo[4,3-c] |
| | pyridin-3-amine hydrochloride |
| | 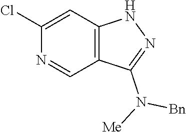 [SMILES: CN(Cc1ccccc1)c1n[nH]c2cc(Cl)ncc12] |
| | SM: N-benzyl-6-chloro-N-methyl-1-(tetrahydro- |
| | 2H-pyran-2-yl)-1H-pyrazolo[4,3-c] |
| | pyridin-3-amine (Preparation 575) |
| | 170 mg, as yellow solid. LCMS m/z = 273.1 [M + H]⁺ |
| 614 | N-benzyl-6-chloro-N-ethyl-1H-pyrazolo[4,3-c] |
| | pyridin-3-amine hydrochloride |
| | 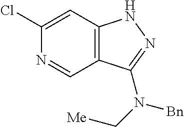 [SMILES: CCN(Cc1ccccc1)c1n[nH]c2cc(Cl)ncc12] |
| | SM: N-benzyl-6-chloro-N-ethyl-1-(tetrahydro-2H- |
| | pyran-2-yl)-1H-pyrazolo[4,3-c] |
| | pyridin-3-amine (Preparation 597) |
| | 400 mg, white solid, LCMS m/z = 287.2 [M + H]⁺ |
| 615 | N-benzyl-6-chloro-1H-pyrazolo[4,3-c]pyridin-3-amine |
| | 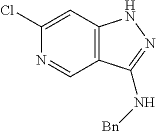 [SMILES: Clc1cc2[nH]nc(NCc3ccccc3)c2cn1] |
| | SM: N-benzyl-6-chloro-1-(tetrahydro-2H-pyran-2-yl)- |
| | 1H-pyrazolo[4,3-c]pyridin-3-amine (Preparation 576) |
| | 1.1 g, crude as yellow solid |
| 616 | 1-(6-chloro-1H-pyrazolo[4,3-c]pyridin-3-yl)piperidin- |
| | 3-amine hydrochloride |
| | 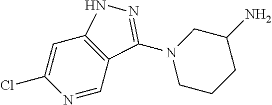 [SMILES: NC1CCCN(c2n[nH]c3cc(Cl)ncc23)C1] |
| | SM: tert-butyl (1-(6-chloro-1-(tetrahydro-2H-pyran-2- |
| | yl)-1H-pyrazolo[4,3-c]pyridin-3-yl) |
| | piperidin-3-yl)carbamate (Preparation 591) |
| | 110 mg, as white solid. LCMS m/z = 252.1 [M + H]⁺ |
| 617 | 1-(6-chloro-1H-pyrazolo[4,3-c]pyridin-3-yl)- |
| | N,N-dimethylpiperidin-4-amine |
| | 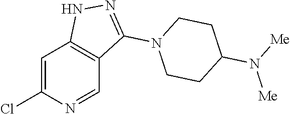 [SMILES: CN(C)C1CCN(c2n[nH]c3cc(Cl)ncc23)CC1] |
| | SM: 1-(6-chloro-1-(tetrahydro-2H-pyran-2-yl)-1H- |
| | pyrazolo[4,3-c]pyridin-3-yl)-N,N- |
| | dimethylpiperidin-4-amine (Preparation 586) |
| | 200 mg, as yellow solid. |
| | LCMS m/z = 280.1 [M + H]⁺ |
| 618 | 6-chloro-3-(2,7-diazaspiro[4.4]nonan-2-yl)-1H-pyrazolo |
| | [4,3-c]pyridine hydrochloride |
| | 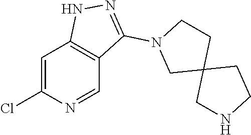 [SMILES: Clc1cc2[nH]nc(N3CCC4(CCNC4)C3)c2cn1] |
| | SM: tert-butyl 7-(6-chloro-1-(tetrahydro-2H-pyran- |
| | 2-yl)-1H-pyrazolo[4,3-c]pyridin-3-yl)-2,7- |
| | diazaspiro[4.4]nonane-2-carboxylate (Preparation 588) |
| 619 | 6-chloro-3-(2,7-diazaspiro[3.5]nonan-2-yl)- |
| | 1H-pyrazolo[4,3-c]pyridine hydrochloride |
| | 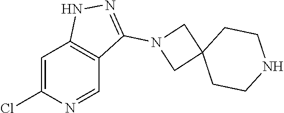 [SMILES: Clc1cc2[nH]nc(N3CC4(CCNCC4)C3)c2cn1] |
| | SM: tert-butyl 2-(6-chloro-1-(tetrahydro- |
| | 2H-pyran-2-yl)-1H-pyrazolo[4,3- |
| | c]pyridin-3-yl)-2,7-diazaspiro[3.5]nonane-7-carboxylate |
| | (Preparation 580) |
| | 288 mg, as yellow solid. ¹H NMR(400 MHz, DMSO- |
| | d₆) δ ppm: 8.66 (s, 1H), 7.38 (s, 1H), 3.93 |
| | (s, 3H), 3.43-3.46 (m, 1H), 3.16 (s, 1H), 3.05 (br s, |
| | 3H), 1.97-2.00 (m, 4H). |
| 620 | 1-(6-chloro-1H-pyrazolo[4,3-c]pyridin-3-yl)-N,N, |
| | 3-trimethylazetidin-3-amine hydrochloride |
| | 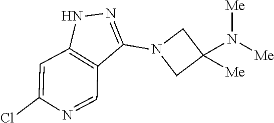 [SMILES: CN(C)C1(C)CN(c2n[nH]c3cc(Cl)ncc23)C1] |
| | SM: 1-(6-chloro-1-(tetrahydro-2H-pyran-2-yl)-1H- |
| | pyrazolo[4,3-c]pyridin-3-yl)-N,N,3- |
| | trimethylazetidin-3-amine (Preparation 577) |
| | 260 mg, crude as yellow oil. LCMS m/z = 266.1 [M + H]⁺ |
| 621 | 3-(6-chloro-1H-pyrazolo[4,3-c]pyridin-3-yl)-3,6- |
| | diazabicyclo[3.1.1]heptane hydrochloride |
| | 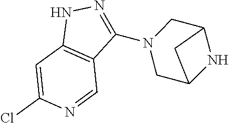 [SMILES: Clc1cc2[nH]nc(N3CC4CC(C3)N4)c2cn1] |
| | SM: tert-butyl 3-(6-chloro-1-(tetrahydro-2H-pyran-2-yl)- |
| | 1H-pyrazolo[4,3-c]pyridin-3-yl)- |
| | 3,6-diazabicyclo[3.1.1]heptane-6-carboxylate |
| | (Preparation 592) |
| | 1.6 g, as yellow solid. LCMS m/z = 250.1 [M + H]⁺ |
| 622 | 1-(6-chloro-1H-pyrazolo[4,3-c]pyridin-3-yl)-N-(4- |
| | methoxybenzyl)-N,4-dimethylpyrrolidin-3- |
| | amine hydrochloride |
| | 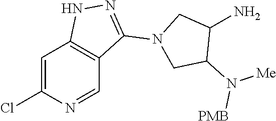 |
| | SM: 1-(6-chloro-1-(tetrahydro-2H-pyran-2-yl)-1H- |
| | pyrazolo[4,3-c]pyridin-3-yl)-N-(4- |
| | methoxybenzyl)-N,4-dimethylpyrrolidin-3-amine |
| | (Preparation 589) |
| | white solid. LCMS m/z = 386.2 [M + H]⁺ |
| 623 | benzyl (1-(6-chloro-1H-pyrazolo[4,3-c]pyridin-3-yl) |
| | pyrrolidin-3-yl)(methyl)carbamate hydrochloride |
| | 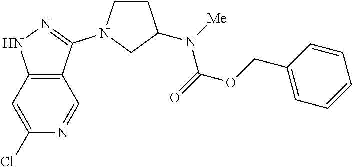 [SMILES: CN(C(=O)OCc1ccccc1)C1CCN(c2n[nH]c3cc(Cl)ncc23)C1] |
| | SM: benzyl (1-(6-chloro-1-(tetrahydro-2H-pyran-2-yl)- |
| | 1H-pyrazolo[4,3-c]pyridin-3-yl) |
| | pyrrolidin-3-yl)(methyl)carbamate (Preparation 590) |
| | yellow solid. ¹H NMR (400 MHz, MeOD-d₄) δ ppm: |
| | 9.45 (s, 1H), 8.01 (s, 1H), 7.72-7.66 (m, 5H), |
| | 5.53-5.46 (m, 2H), 5.35-5.31 (m, 1H), 4.24-4.19 |
| | (m, 2H), 4.06-4.00 (m, 2H), 3.65 (s, 3H), 2.70-2.64 (m, 2H). |

### Preparation 624: 1-(6-chloro-1H-pyrazolo[4,3-c]pyridin-3-yl)-5-methylpyrrolidin-3-ol trifluoroacetate

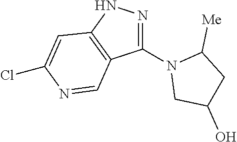 [SMILES: CC1CC(O)CN1c1n[nH]c2cc(Cl)ncc12]

To a solution of 1-(6-chloro-1-(tetrahydro-2H-pyran-2-yl)-1H-pyrazolo[4,3-c]pyridin-3-yl)-5-methylpyrrolidin-3-ol (Preparation 565, 179 mg, 0.531 mmol) in DCM (4 mL) was added TFA (1.5 g, 13.06 mmol) and the reaction mixture was stirred at 25° C. for 2 h. The mixture was treated with H₂O (10 mL) and extracted with DCM (10 mL×3). The aqueous phase was lyophilized to give 1-(6-chloro-1H-pyrazolo[4,3-c]pyridin-3-yl)-5-methylpyrrolidin-3-ol trifluoroacetate (150 mg, crude) as yellow oil. LCMS m/z=253.0 [M+H]⁺

### Preparation 625: 1-(6-chloro-1H-pyrazolo[4,3-c]pyridin-3-yl)-4-methylpyrrolidin-3-amine trifluoroacetate

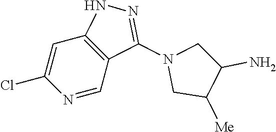 [SMILES: CC1CN(c2n[nH]c3cc(Cl)ncc23)CC1N]

To solution of tert-butyl (1-(6-chloro-1-(tetrahydro-2H-pyran-2-yl)-1H-pyrazolo[4,3-c]pyridin-3-yl)-4-methylpyrrolidin-3-yl)carbamate (Preparation 567, 170 mg, 0.39 mmol) in DCM (4 mL) was added TFA (1.5 g, 13.06 mmol) and the reaction mixture stirred at 25° C. for 6 h. The mixture was concentrated to give 1-(6-chloro-1H-pyrazolo[4,3-c]pyridin-3-yl)-4-methylpyrrolidin-3-amine trifluoroacetate (320.0 mg, crude) as a brown gum. ¹H NMR: (500 MHz, MeOD-d₄) δ ppm: 8.86 (s, 1H), 7.39 (s, 1H), 4.11-4.01 (m, 3H), 3.99-3.94 (m, 2H,) 3.71-3.41 (m, 1H), 1.29-1.27 (m, 3H).

### Preparation 626: 6-chloro-3-(3,3-difluoro-4-methoxypyrrolidin-1-yl)-1H-pyrazolo[4,3-c]pyridine trifluoroacetate

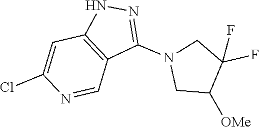 [SMILES: COC1CN(c2n[nH]c3cc(Cl)ncc23)CC1(F)F]

To a solution of 6-chloro-3-(3,3-difluoro-4-methoxypyrrolidin-1-yl)-1-(tetrahydro-2H-pyran-2-yl)-1H-pyrazolo[4,3-c]pyridine (Preparation 570, 60.0 mg, 161 mmol) in DCM (4 mL) was added TFA (2.0 mL) and the reaction was stirred at 25° C. for 16 h. The mixture was concentrated and purified by prep-TLC (PE/EtOAc=1/1) to give 6-chloro-3-(3,3-difluoro-4-methoxypyrrolidin-1-yl)-1H-pyrazolo[4,3-c]pyridine trifluoroacetate (45 mg, crude) as a white solid. LCMS m/z=289.0 [M+H]⁺

### Preparation 627: (S)-1-(6-chloro-1H-pyrazolo[4,3-c]pyridin-3-yl)-N,N-dimethylpyrrolidin-3-amine

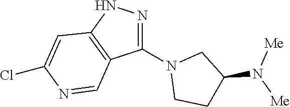 [SMILES: CN(C)[C@H]1CCN(c2n[nH]c3cc(Cl)ncc23)C1]

To a solution of (3S)-1-(6-chloro-1-(tetrahydro-2H-pyran-2-yl)-1H-pyrazolo[4,3-c]pyridin-3-yl)-N,N-dimethylpyrrolidin-3-amine (Preparation 569, 650 mg, 1.86 mmol) in DCM (6 mL) was added TFA (1.5 mL) and the reaction was stirred at 25° C. for 26 h. The mixture was adjusted pH=7 using NH₃H₂O. The crude product was purified by prep-HPLC-A (12% to 42% MeCN) to give (S)-1-(6-chloro-1H-pyrazolo[4,3-c]pyridin-3-yl)-N,N-dimethylpyrrolidin-3-amine (342.5 mg, 69.4% yield) as a white solid. LCMS m/z=266.1 [M+H]⁺

### Preparation 628: 1-(6-chloro-1H-pyrazolo[4,3-c]pyridin-3-yl)azetidin-3-amine trifluoroacetate

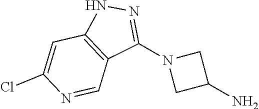 [SMILES: NC1CN(c2n[nH]c3cc(Cl)ncc23)C1]

To a solution of tert-butyl (1-(6-chloro-1-(tetrahydro-2H-pyran-2-yl)-1H-pyrazolo[4,3-c]pyridin-3-yl)azetidin-3-yl)carbamate (Preparation 583, 490 mg, 1.20 mmol) in HFIPA (10 mL) was added TFA (547.9 mg, 4.81 mmol) and the reaction was stirred at 25° C. for 16 h. The reaction was concentrated to give 1-(6-chloro-1H-pyrazolo[4,3-c]pyridin-3-yl)azetidin-3-amine trifluoroacetate (270 mg, crude) as a yellow solid. LCMS m/z=224.0 [M+H]⁺

### Preparation 629: 9-(6-chloro-1H-pyrazolo[4,3-c]pyridin-3-yl)-6-oxa-2,9-diazaspiro[4.5]decane trifluoroacetate

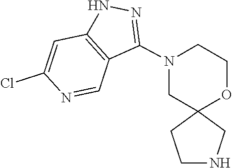 [SMILES: Clc1cc2[nH]nc(N3CCOC4(CCNC4)C3)c2cn1]

9-(6-Chloro-1H-pyrazolo[4,3-c]pyridin-3-yl)-6-oxa-2,9-diazaspiro[4.5]decane trifluoroacetate was obtained as a yellow solid, from tert-butyl 9-(6-chloro-1-(tetrahydro-2H-pyran-2-yl)-1H-pyrazolo[4,3-c]pyridin-3-yl)-6-oxa-2,9-diazaspiro[4.5]decane-2-carboxylate (Preparation 593) following the procedure described in Preparation 628. LCMS m/z=294.2 [M+H]⁺

### Preparation 630: 6-chloro-3-(2,6-diazaspiro[3.4]octan-2-yl)-1H-pyrazolo[4,3-c]pyridine

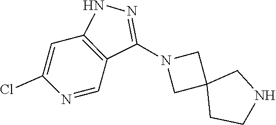 [SMILES: Clc1cc2[nH]nc(N3CC4(CCNC4)C3)c2cn1]

To a solution of tert-butyl 2-(6-chloro-1-(tetrahydro-2H-pyran-2-yl)-1H-pyrazolo[4,3-c]pyridin-3-yl)-2,6-diazaspiro[3.4]octane-6-carboxylate (Preparation 587, 200 mg, 0.446 mmol) in HFIPA (5 mL) was added TFA (0.5 mL) and the reaction was stirred at 25° C. for 5 h. The mixture was concentrated and adjusted to pH 7-8 with NH₃·H₂O, then purified by prep-HPLC-C (13% to 43% MeCN) to give 6-chloro-3-(2,6-diazaspiro[3.4]octan-2-yl)-1H-pyrazolo[4,3-c]pyridine (35.0 mg, 29.7% yield) as a white solid. LCMS m/z=264.1 [M+H]⁺

### Preparation 631: 6-chloro-N-methyl-N-(1-methylpyrrolidin-3-yl)-1H-pyrazolo[4,3-c]pyridin-3-amine

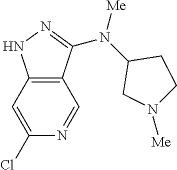 [SMILES: CN1CCC(N(C)c2n[nH]c3cc(Cl)ncc23)C1]

To a solution of 6-chloro-N-methyl-N-(pyrrolidin-3-yl)-1H-pyrazolo[4,3-c]pyridin-3-amine hydrochloride (Preparation 600, 100 mg, 0.347 mmol) in MeOH (5.0 mL) was added NaBH₃CN (109 mg, 1.74 mmol) and (CH₂O)ₙ(1.3 g, 1.04 mmol) and the reaction stirred at 25° C. for 16 h. The mixture was concentrated and purified by prep-HPLC-L (30% to 60% MeCN) to give 6-chloro-N-methyl-N-(1-methylpyrrolidin-3-yl)-1H-pyrazolo[4,3-c]pyridin-3-amine (60.0 mg, 65.1% yield) as a yellow solid. LCMS m/z=266.1 [M+H]⁺

### Preparations 632 to 643

The compounds in the following table were prepared from the appropriate amine and (CH₂O)ₙ, following a similar procedure to that described in Preparation 631.

| Preparation | |
|---|---|
| No. | Name, Structure, Starting Materials (SM), Data |
| 632 | 6-chloro-3-(9-methyl-3,9-diazabicyclo |
| | [3.3.1]nonan-3-yl)-1H-pyrazolo[4,3- |
| | c]pyridine |
| | 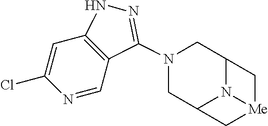 |
| | SM: 3-(3,9-diazabicyclo[3.3.1]nonan-3- |
| | yl)-6-chloro-1H-pyrazolo[4,3- |
| | c]pyridine hydrochloride (Preparation 601) |
| | Prep-HPLC-L (25% to 45%). |
| | 60 mg, 31.7% as a yellow solid. |
| | LCMS m/z = 292.1[M + H]⁺ |
| 633 | (R)-1-(6-chloro-1H-pyrazolo[4,3-c]pyridin-3-yl)- |
| | N,N-dimethylpyrrolidin-3-amine |
| | 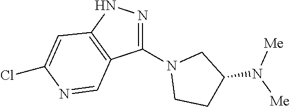 [SMILES: CN(C)[C@@H]1CCN(c2n[nH]c3cc(Cl)ncc23)C1] |
| | SM: (R)-1-(6-chloro-1H-pyrazolo[4,3-c]pyridin-3-yl) |
| | pyrrolidin-3-amine hydrochloride (Preparation 602) |
| | Prep-HPLC-P (15% to 45% MeCN) |
| | 250 mg, 33.4% yield as a yellow solid. |
| | LCMS m/z = 266.1[M + H]⁺ |
| 634 | 1-(6-chloro-1H-pyrazolo[4,3-c]pyridin-3-yl)- |
| | N,N,4-trimethylpyrrolidin-3-amine |
| | 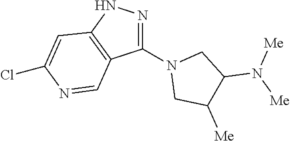 [SMILES: CC1CN(c2n[nH]c3cc(Cl)ncc23)CC1N(C)C] |
| | SM: 1-(6-chloro-1H-pyrazolo[4,3-c]pyridin- |
| | 3-yl)-4-methylpyrrolidin-3- |
| | amine trifluoroacetate (Preparation 625) |
| | prep-HPLC-F (30% to 60% MeCN) |
| | 36.0 mg, 27.0% yield as yellow solid. ¹H NMR: |
| | (400 MHz, MeOD-d₄) δ |
| | ppm: 8.83-8.82 (m, 1H), 7.28-7.27 (m, 1H), |
| | 3.95-3.91 (m, 1H), 3.80-3.78 (m, 3H), 3.77-3.65 (m, 1H), |
| | 2.82-2.76 (m, 1H), 2.32 (s, 6H) 1.11-1.09 (m, |
| | 3H). |
| 635 | 1-(6-chloro-1H-pyrazolo[4,3-c]pyridin-3-yl)- |
| | N,N,5-trimethylpyrrolidin-3-amine |
| | 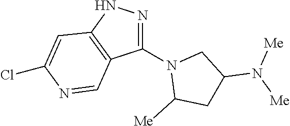 [SMILES: CC1CC(N(C)C)CN1c1n[nH]c2cc(Cl)ncc12] |
| | SM: 1-(6-chloro-1H-pyrazolo[4,3-c]pyridin- |
| | 3-yl)-5-methylpyrrolidin-3- |
| | amine hydrochloride (Preparation 606) |
| | Prep-HPLC-I (90% to 98% MeCN) |
| | 20 mg, 24.1% yield as a yellow solid. |
| | LCMS m/z = 280.2 [M + H]⁺ |
| 636 | Cis-rac-1-(6-chloro-1H-pyrazolo[4,3-c]pyridin-3- |
| | yl)-N,N,4-trimethylpyrrolidin-3-amine |
| | 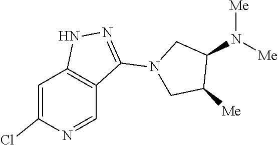 [SMILES: C[C@H]1CN(c2n[nH]c3cc(Cl)ncc23)C[C@H]1N(C)C] |
| | SM: cis-rac-1-(6-chloro-1H-pyrazolo[4,3-c]pyridin-3-yl)-4- |
| | methylpyrrolidin-3-amine hydrochloride (Preparation 608) |
| | prep-HPLC-C (16% to 46% MeCN) |
| | 35.0 mg, 45.1% yield as a yellow solid. |
| | LCMS m/z = 280.2 [M + H]⁺ |
| 637 | 1-(6-chloro-1H-pyrazolo[4,3-c]pyridin-3-yl)-4,4- |
| | difluoro-N,N-dimethylpyrrolidin-3-amine |
| | 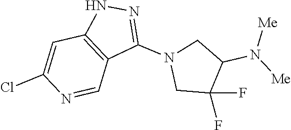 [SMILES: CN(C)C1CN(c2n[nH]c3cc(Cl)ncc23)CC1(F)F] |
| | SM: 1-(6-chloro-1H-pyrazolo[4,3-c]pyridin- |
| | 3-yl)-4,4-difluoropyrrolidin-3- |
| | amine hydrochloride (Preparation 609) |
| | Prep-HPLC-P (30% to 60% MeCN) |
| | 60 mg, 49.5% yield as a yellow solid. ¹H NMR: |
| | (400 MHz, CDCl₃) δ: ppm |
| | 9.19 (s, 1H), 8.76 (s, 1H), 7.23 (s, 1H), |
| | 4.13-4.04 (m, 3H), 3.76-3.73 (m, |
| | 1H), 3.28-3.25 (m, 1H), 2.52 (s, 6H). |
| 638 | 1-(6-chloro-1H-pyrazolo[4,3-c]pyridin-3-yl)- |
| | N,N-dimethylazetidin-3-amine |
| | 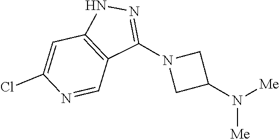 [SMILES: CN(C)C1CN(c2n[nH]c3cc(Cl)ncc23)C1] |
| | SM: 1-(6-chloro-1H-pyrazolo[4,3-c]pyridin-3-yl) |
| | azetidin-3-amine trifluoroacetate (Preparation 628) |
| | Prep-HPLC-U (0% to 15% MeCN) 100 mg, |
| | 34.2% yield as a yellow solid. |
| | ¹H NMR: (400 MHz, CDCl₃) δ: ppm 8.55 (s 1H), 8.50 |
| | (s 1H), 7.16 (s, 1H), 4.27-4.23 (m, 2H), 4.13-4.09 (m, |
| | 2H), 3.54-3.48 (m, 1H), 2.36-2.33 (m, 6H). |
| 639 | 6-chloro-3-(7-methyl-2,7-diazaspiro[4.4]nonan- |
| | 2-yl)-1H-pyrazolo[4,3-c]pyridine |
| | 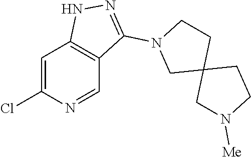 [SMILES: CN1CCC2(CCN(c3n[nH]c4cc(Cl)ncc34)C2)C1] |
| | SM: 6-chloro-3-(2,7-diazaspiro[4.4]nonan-2-yl)-1H-pyrazolo |
| | [4,3-c]pyridine hydrochloride (Preparation 618) |
| | Prep-HPLC-C (23% to 53% MeCN) |
| | 120 mg, 25.4% as white solid. LCMS m/z = 292.1[M + H]⁺ |
| 640 | 6-chloro-3-(7-methyl-2,7-diazaspiro[3.5]nonan- |
| | 2-yl)-1H-pyrazolo[4,3-c]pyridine |
| | 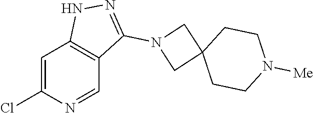 [SMILES: CN1CCC2(CC1)CN(c1n[nH]c3cc(Cl)ncc13)C2] |
| | SM: 6-chloro-3-(2, 7-diazaspiro[3.5]nonan-2-yl)-1H-pyrazolo |
| | [4,3-c]pyridine hydrochloride (Preparation 619) |
| | prep-HPLC-F (30% to 60% MeCN) |
| | 77.0 mg, 29.3% yield as a white solid. ¹H NMR: |
| | (500 MHz, CDCl₃) δ ppm: |
| | 8.62 (s, 1H), 7.19 (s, 1H), 3.94-3.93 (m, 4H), 2.28 |
| | (s, 3H), 1.92-1.91 (m, 4H), 1.58 (s, 4H). |
| 641 | 9-(6-chloro-1H-pyrazolo[4,3-c]pyridin-3-yl)-2- |
| | methyl-6-oxa-2,9-diazaspiro[4.5]decane |
| | 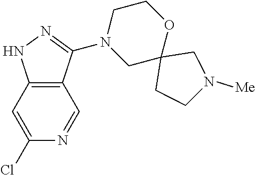 [SMILES: CN1CCC2(C1)CN(c1n[nH]c3cc(Cl)ncc13)CCO2] |
| | SM: 9-(6-chloro-1H-pyrazolo[4,3-c] |
| | pyridin-3-yl)-6-oxa-2,9- |
| | diazaspiro[4.5]decane trifluoroacetate (Preparation 629) |
| | Prep-HPLC-U (7% to 27% MeCN) |
| | 110 mg, 70.0% yield as a yellow solid. |
| | LCMS m/z = 308.0 [M + H]⁺ |
| 642 | 6-chloro-3-(6-methyl-2,6-diazaspiro[3.4]octan-2-yl)- |
| | 1H-pyrazolo[4,3-c]pyridine |
| | 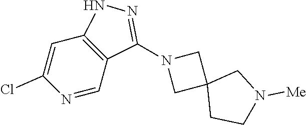 [SMILES: CN1CCC2(C1)CN(c1n[nH]c3cc(Cl)ncc13)C2] |
| | SM: 6-chloro-3-(2,6-diazaspiro[3.4]octan-2-yl)- |
| | 1H-pyrazolo[4,3-c]pyridine |
| | (Preparation 630) |
| | Prep-HPLC-C (17% to 47% MeCN) |
| | 25 mg, 67.8% as yellow solid. |
| | LCMS m/z = 278.1[M + H]⁺ |
| 643 | 1-(6-chloro-1H-pyrazolo[4,3-c]pyridin-3-yl)- |
| | N,N-dimethylpiperidin-3-amine |
| | 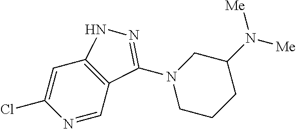 [SMILES: CN(C)C1CCCN(c2n[nH]c3cc(Cl)ncc23)C1] |
| | SM: 1-(6-chloro-1H-pyrazolo[4,3-c] |
| | pyridin-3-yl)piperidin-3-amine |
| | hydrochloride (Preparation 616) |
| | Prep-HPLC-C (15% to 42% MeCN) |
| | 50 mg, 51.5% as white solid. LCMS m/z = 280.2 [M + H]⁺ |

### Preparation 644: 3-(6-chloro-1H-pyrazolo[4,3-c]pyridin-3-yl)-6-ethyl-3,6-diazabicyclo[3.1.1]heptane

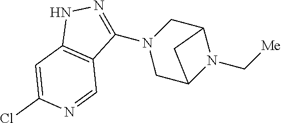 [SMILES: CCN1C2CC1CN(c1n[nH]c3cc(Cl)ncc13)C2]

To a solution of 3-(6-chloro-1H-pyrazolo[4,3-c]pyridin-3-yl)-3,6-diazabicyclo[3.1.1]heptane (Preparation 621, 300 mg, 1.20 mmol) and NaBH₃CN (377.5 mg, 6.01 mmol) in MeOH (20 mL) was added CH₃CHO (1.1 g, 24.0 mmol) and the reaction was stirred at 25° C. for 2 h under N₂. The mixture was concentrated and purified by Prep-HPLC-Q (5% to 35% MeCN) to give 3-(6-chloro-1H-pyrazolo[4,3-c]pyridin-3-yl)-6-ethyl-3,6-diazabicyclo[3.1.1]heptane (150 mg, 45.0% yield) as a yellow solid. LCMS m/z=278.1 [M+H]⁺

### Preparation 645: 6-chloro-3-(3,3-difluorocyclobutyl)-1H-pyrazolo[4,3-c]pyridine

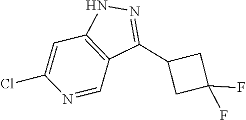 [SMILES: FC1(F)CC(c2n[nH]c3cc(Cl)ncc23)C1]

TFA (1.42 g, 12.47 mmol) was added to a solution of 6-chloro-3-(3,3-difluorocyclobutyl)-1-(tetrahydro-2H-pyran-2-yl)-1H-pyrazolo[4,3-c]pyridine (Preparation 599, 174 mg, 0.531 mmol) in DCM (9.55 mL) at 0° C. The reaction was warmed to rt and stirred overnight. The solvent was removed, the residue was dissolved in EtOAc and washed with NaHCO₃ (aq). The organic layer was concentrated in vacuo and residue was purified by silica gel chromatography eluting with heptane to EtOAc:EtOH 3:1 to give 6-chloro-3-(3,3-difluorocyclobutyl)-1H-pyrazolo[4,3-c]pyridine, 57 mg of white solid. LCMS m/z=244 [M+H]⁺

### Preparation 646: 3-bromo-6-chloro-1-(2-(1,1-difluoroethyl)-6-ethylpyrimidin-4-yl)-1H-pyrazolo[4,3-c]pyridine

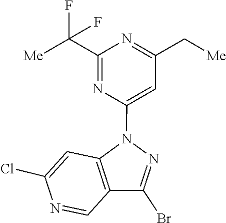 [SMILES: CCc1cc(-n2nc(Br)c3cnc(Cl)cc32)nc(C(C)(F)F)n1]

To a solution of 3-bromo-6-chloro-1H-pyrazolo[4,3-c]pyridine (1.5 g, 6.45 mmol) in THE (20 mL) was added 4-chloro-2-(1,1-difluoroethyl)-6-ethylpyrimidine (Preparation 390, 1.6 g, 7.74 mmol) and Cs₂CO₃ (6.3 g, 19.4 mmol) and the reaction was stirred at 60° C. for 12 h. The mixture was diluted with H₂O (50 mL), concentrated and extracted with EtOAc (50 mL×3). The combined organic layers were washed with brine (50 mL), dried over Na₂SO₄, filtered and concentrated under vacuum. The crude product was purified by silica gel column chromatography (PE/EtOAc=1/0 to 3/1) to give 3-bromo-6-chloro-1-(2-(1,1-difluoroethyl)-6-ethylpyrimidin-4-yl)-1H-pyrazolo[4,3-c]pyridine (2.2 g, 84.7% yield) as a white solid. ¹H NMR: (400 MHz, CDCl₃) δ ppm: 8.81 (d, J=1.2 Hz, 1H), 7.59 (s, 1H), 7.85 (s, 1H), 2.96 (q, J=7.6 Hz, 2H), 2.14 (d, J=18.4 Hz, 3H), 1.41 (d, J=7.6 Hz, 3H).

### Preparation 647: 3-bromo-6-chloro-1-(2-(1,1-difluoroethyl)-6-methylpyrimidin-4-yl)-1H-pyrazolo[4,3-c]pyridine

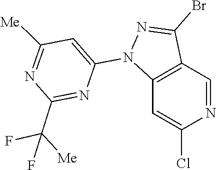 [SMILES: Cc1cc(-n2nc(Br)c3cnc(Cl)cc32)nc(C(C)(F)F)n1]

3-Bromo-6-chloro-1-(2-(1,1-difluoroethyl)-6-methylpyrimidin-4-yl)-1H-pyrazolo[4,3-c]pyridine was obtained as a yellow solid, 1.52 g, 90.9% yield from 3-bromo-6-chloro-1H-pyrazolo[4,3-c]pyridine and 4-chloro-2-(1,1-difluoroethyl)-6-methylpyrimidine (Preparation 218) following the procedure described in Preparation 646. ¹H NMR: (500 MHz, CDCl₃) δ: ppm 8.81 (s, 1H), 8.74 (s, 1H), 7.85 (s, 1H), 2.70 (s, 3H), 2.18-2.07 (m, 3H).

### Preparation 648: 3-bromo-6-chloro-1-(6-(1,1-difluoroethyl)pyrazin-2-yl)-1H-pyrazolo[4,3-c]pyridine

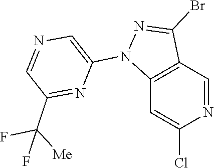 [SMILES: CC(F)(F)c1cncc(-n2nc(Br)c3cnc(Cl)cc32)n1]

To a solution of 3-bromo-6-chloro-1H-pyrazolo[4,3-c]pyridine (500 mg, 2.15 mmol) in DMF (10 mL) was added 2-chloro-6-(1,1-difluoroethyl)pyrazine (384.1 mg, 2.15 mmol) and Cs₂CO₃ (700.8 mg, 2.15 mmol). The resulting mixture was stirred at 100° C. for 16 h. The mixture was quenched with H₂O (20 mL) and extracted with EtOAc (20 mL×2). The combined organic phase was washed with brine (20 mL×2), dried over Na₂SO₄ and filtered. The filtrate was concentrated under reduced pressure and the residue was purified by chromatography on silica gel (PE/EtOAc=2/1) to give 3-bromo-6-chloro-1-(6-(1,1-difluoroethyl)pyrazin-2-yl)-1H-pyrazolo[4,3-c]pyridine (620 mg, 77.0% yield) a white solid. ¹H NMR (400 MHz, CDCl₃) δ: ppm 9.47 (s, 1H), 8.87 (s, 1H), 8.85 (s, 1H), 8.49 (s, 1H), 2.15 (t, J=18.8 Hz, 3H).

### Preparation 649: tert-butyl (1-(6-chloro-1H-pyrazolo[4,3-c]pyridin-3-yl)-5-methylpyrrolidin-3-yl)carbamate

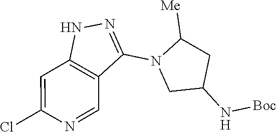 [SMILES: CC1CC(NC(=O)OC(C)(C)C)CN1c1n[nH]c2cc(Cl)ncc12]

To a solution of 1-(6-chloro-1H-pyrazolo[4,3-c]pyridin-3-yl)-5-methylpyrrolidin-3-amine hydrochloride (Preparation 606, 135 mg, 0.54 mmol) in DCM (10 mL) was added Boc₂O (117.1 mg, 0.54 mmol) and TEA (5.4 mg, 0.054 mmol) and the reaction was stirred at 25° C. for 16 h. The mixture was concentrated and the residue was purified by chromatography on silica gel (PE/EtOAc=1/1) to give tert-butyl (1-(6-chloro-1H-pyrazolo[4,3-c]pyridin-3-yl)-5-methylpyrrolidin-3-yl)carbamate (80 mg, 42.4% yield) as a yellow oil. LCMS m/z=352.1 [M+H]⁺

### Preparation 650: 6-chloro-1-(2-(1,1-difluoroethyl)pyrimidin-4-yl)-3-(9-methyl-3,9-diazabicyclo[3.3.1]nonan-3-yl)-1H-pyrazolo[4,3-c]pyridine

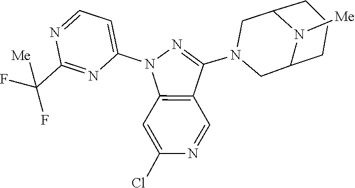 [SMILES: CN1C2CCCC1CN(c1nn(-c3ccnc(C(C)(F)F)n3)c3cc(Cl)ncc13)C2]

To a solution of 6-chloro-3-(9-methyl-3,9-diazabicyclo[3.3.1]nonan-3-yl)-1H-pyrazolo[4,3-c]pyridine (Preparation 632, 50 mg, 0.171 mmol) in DMF (2 mL) was added 4-chloro-2-(1,1-difluoroethyl)pyrimidine (33.7 mg, 0.188 mmol) and Cs₂CO₃ (111.7 mg, 0.343 mmol) and the reaction mixture was stirred at 100° C. for 5 h. The mixture was quenched with H₂O (20 mL) and extracted with EtOAc (20 mL×2). The combined organic phase was washed with brine (20 mL×2), dried over Na₂SO₄ and filtered. The filtrate was concentrated under reduced pressure and the residue was purified by chromatography on silica gel eluting with 100% EtOAc to give 6-chloro-1-(2-(1,1-difluoroethyl)pyrimidin-4-yl)-3-(9-methyl-3,9-diazabicyclo[3.3.1]nonan-3-yl)-1H-pyrazolo[4,3-c]pyridine (30 mg, 40.4% yield) as a yellow solid. 1H NMR: (500 MHz, CDCl₃) δ: ppm 8.92 (s, 1H), 8.80-8.78 (m, 2H), 7.80 (d, J=6.0 Hz, 1H), 4.07 (br s, 3H), 3.37 (br s, 1H), 2.35-2.28 (m, 4H), 2.18-2.08 (m, 6H), 1.89 (br s, 2H), 1.72 (br s, 2H).

### Preparations 651 to 663

The compounds in the following table were prepared from the appropriate 6-chloro-1H-pyrazolo[4,3-c]pyridine and 4-chloro-2-(1,1-difluoroethyl)pyrimidine (Preparation 184), following a similar procedure to that described in Preparation 650.

| Preparation | |
|---|---|
| No | Name, Structure, Starting Materials (SM), Data |
| 651^{A} | 1-(6-chloro-1-(2-(1,1-difluoroethyl) |
| | pyrimidin-4-yl)-1H-pyrazolo[4,3- |
| | c]pyridin-3-yl)-N,N-dimethylpyrrolidin-3-amine |
| | 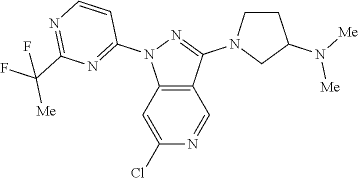 [SMILES: CN(C)C1CCN(c2nn(-c3ccnc(C(C)(F)F)n3)c3cc(Cl)ncc23)C1] |
| | SM: 1-(6-chloro-1H-pyrazolo[4,3-c]pyridin-3-yl)- |
| | N,N-dimethylpyrrolidin-3-amine |
| | (Preparation 285) |
| | (90.0 mg, 58.6% yield) as a light yellow solid. |
| | ¹H NMR: (400 MHz, CDCl₃) δ ppm: 8.85 (s, 1H), |
| | 8.75 (d, J = 5.6 Hz, 1H), 8.70 (s, 1H), 7.80 (d, J = 5.6 |
| | Hz, 1H), 3.92-3.97 (m, 2H), 3.75-3.78 (m, 1H), 3.53- |
| | 3.58 (m, 1H), 2.96-2.99 (m, 1H), 2.37 (s, 6H), |
| | 2.32-2.35 (m, 1H), 2.05-2.18 (m, 4H) |
| 652^{A} | 6-chloro-1-(2-(1,1-difluoroethyl)pyrimidin-4-yl)- |
| | N-methyl-N-(1-methylpyrrolidin-3-yl)- |
| | 1H-pyrazolo[4,3-c]pyridin-3-amine |
| | 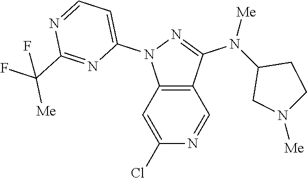 [SMILES: CN1CCC(N(C)c2nn(-c3ccnc(C(C)(F)F)n3)c3cc(Cl)ncc23)C1] |
| | SM: 6-chloro-N-methyl-N-(1-methylpyrrolidin- |
| | 3-yl)-1H-pyrazolo[4,3- |
| | c]pyridin-3-amine (Preparation 631) |
| | prep-HPLC-C (34% to 64%) 30 mg, as a white solid. |
| | LCMS m/z = 408.1 [M + H]⁺ |
| 653 | 1-(6-chloro-1-(2-(1,1-difluoroethyl)pyrimidin- |
| | 4-yl)-1H-pyrazolo[4,3- |
| | c]pyridin-3-yl)-5-methylpyrrolidin-3-ol |
| | 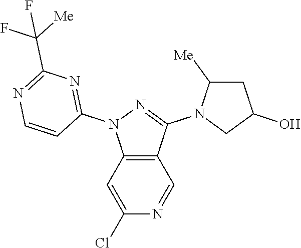 [SMILES: CC1CC(O)CN1c1nn(-c2ccnc(C(C)(F)F)n2)c2cc(Cl)ncc12] |
| | SM: 1-(6-chloro-1H-pyrazolo[4,3-c]pyridin- |
| | 3-yl)-5-methylpyrrolidin-3-ol |
| | trifluoroacetate (Preparation 624) |
| | 15.0 mg, as a white solid. LCMS m/z = |
| | 395.1[M + H]⁺ |
| 654 | 6-chloro-3-(3,3-difluoro-4-methoxypyrrolidin-1- |
| | yl)-1-(2-(1,1-difluoroethyl)pyrimidin-4-yl)-1H- |
| | pyrazolo[4,3-c]pyridine |
| | 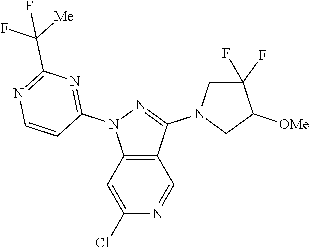 [SMILES: COC1CN(c2nn(-c3ccnc(C(C)(F)F)n3)c3cc(Cl)ncc23)CC1(F)F] |
| | SM: 6-chloro-3-(3,3-difluoro-4-methoxypyrrolidin- |
| | 1-yl)-1H-pyrazolo[4,3- |
| | c]pyridine trifluoroacetate (Preparation 626) |
| | 25.0 mg, 55.8% yield as a yellow solid. |
| | LCMS m/z = 431.1[M + H]⁺ |
| 655 | 6-chloro-1-(2-(1,1-difluoroethyl)pyrimidin-4- |
| | yl)-3-(4-methoxy-2-methylpyrrolidin- |
| | 1-yl)-1H-pyrazolo[4,3-c]pyridine |
| | 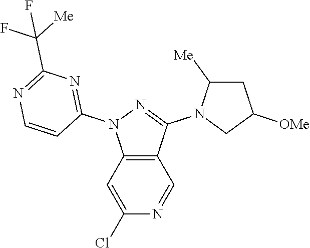 [SMILES: COC1CC(C)N(c2nn(-c3ccnc(C(C)(F)F)n3)c3cc(Cl)ncc23)C1] |
| | SM: 6-chloro-3-(4-methoxy-2-methylpyrrolidin- |
| | 1-yl)-1H-pyrazolo[4,3-c]pyridine |
| | hydrochloride (Preparation 605) |
| | 20 mg, 43.5% yield as a white solid. |
| | LCMS m/z = 409.1[M + H]⁺ |
| 656^{A} | 6-chloro-1-(2-(1,1-difluoroethyl)pyrimidin- |
| | 4-yl)-3-(3-methoxy-4- |
| | methylpyrrolidin-1-yl)-1H-pyrazolo[4,3-c]pyridine |
| | 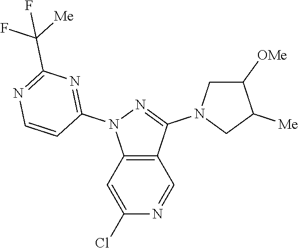 [SMILES: COC1CN(c2nn(-c3ccnc(C(C)(F)F)n3)c3cc(Cl)ncc23)CC1C] |
| | SM: 6-chloro-3-(3-methoxy-4-methylpyrrolidin- |
| | 1-yl)-1H-pyrazolo[4,3- |
| | c]pyridine hydrochloride (Preparation 604) |
| | 70.0 mg, 65.2% yield as a white solid. |
| | LCMS m/z = 49.1[M + H]⁺ |
| 657 | 1-(6-chloro-1-(2-(1,1-difluoroethyl)pyrimidin-4-yl)- |
| | 1H-pyrazolo[4,3-c]pyridin-3-yl)-N-(4- |
| | methoxybenzyl)-N,4-dimethylpyrrolidin-3-amine |
| | 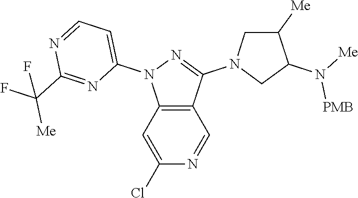 |
| | SM: 1-(6-chloro-1H-pyrazolo[4,3-c]pyridin-3-yl)-N- |
| | (4-methoxybenzyl)-N,4-dimethylpyrrolidin-3- |
| | amine hydrochloride (Preparation 622) |
| | 176 mg, 32% as yellow solid. ¹HNMR (400 MHz, |
| | CDCl₃) δ ppm: 8.82 (s, 1H), 8.74 (d, J = 6.0 |
| | Hz, 1H), 8.70 (s, 1H), 7.79 (d, J = 6.0 Hz, 1H), 7.29 (m, |
| | 2H), 6.89-6.90 (m, 2H), 3.89-3.93 (m, 2H), 3.83 (s, |
| | 3H), 3.64-3.68 (m, 2H), 3.14-3.16 (m, 1H), 3.04- |
| | 3.05 (m, 1H), 2.66-2.69 (m, 1H), 2.10-2.17 (m, |
| | 7H), 1.20 (d, J = 6.5 Hz, 3H) |
| 658 | 1-(6-chloro-1-(2-(1,1-difluoroethyl)pyrimidin-4-yl)- |
| | 1H-pyrazolo[4,3-c]pyridin- |
| | 3-yl)-N,N,4-trimethylpyrrolidin-3-amine |
| | 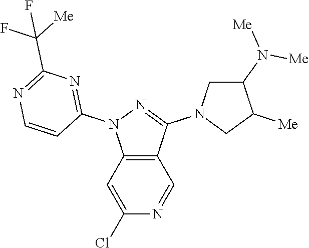 [SMILES: CC1CN(c2nn(-c3ccnc(C(C)(F)F)n3)c3cc(Cl)ncc23)CC1N(C)C] |
| | SM: 1-(6-chloro-1H-pyrazolo[4,3-c]pyridin-3-yl)-N,N,4- |
| | trimethylpyrrolidin-3-amine (Preparation 634) |
| | 33.0 mg, crude as a white solid. ¹H NMR |
| | (400 MHz, CDCl₃) δ ppm: 8.86 |
| | (d, J = 4.4 Hz, 1H), 8.76 (d, J = 4.4 Hz, 1H), |
| | 8.71 (s, 1H), 7.80-7.82 (m, 1H), |
| | 3.37-4.02 (m, 4H), 2.67-2.97 (m, 1H), 2.54-2.57 |
| | (m, 1H), 2.43 (s, 3H), 2.34 |
| | (s, 3H), 2.15 (t, J = 18.4 Hz, 3H), 1.12-1.25 (m, 3H). |
| 659 | tert-butyl (1-(6-chloro-1-(2-(1,1-difluoroethyl) |
| | pyrimidin-4-yl)-1H-pyrazolo[4,3-c]pyridin- |
| | 3-yl)-5-methylpyrrolidin-3-yl)carbamate |
| | 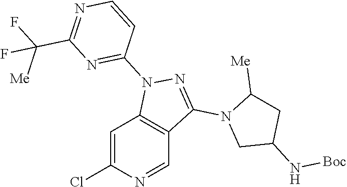 [SMILES: CC1CC(NC(=O)OC(C)(C)C)CN1c1nn(-c2ccnc(C(C)(F)F)n2)c2cc(Cl)ncc12] |
| | SM: tert-butyl (1-(6-chloro-1H-pyrazolo |
| | [4,3-c]pyridin-3-yl)-5- |
| | methylpyrrolidin-3-yl)carbamate (Preparation 649) |
| | 65 mg, 66.1% as a yellow oil. ¹H NMR: |
| | (500 MHz, CDCl₃ ) δ: ppm 8.82 (s, |
| | 1H), 8.76 (d, J = 5.5 Hz, 1H), 8.71 (s, 1H), |
| | 7.78 (d, J = 5.5 Hz, 1H), 4.27-4.41 |
| | (m, 2H), 3.67-3.70 (m, 1H), 3.14 (br s, 1H), |
| | 2.01-2.17 (m, 3H), 1.74 (br s, |
| | 1H), 1.46-1.50 (m, 9H), 1.25-1.27 (m, |
| | 3H), 0.84-0.88 (m, 2H). |
| 660 | 1-(6-chloro-1-(2-(1,1-difluoroethyl)pyrimidin-4- |
| | yl)-1H-pyrazolo[4,3-c]pyridin-3-yl)- |
| | N,N,5-trimethylpyrrolidin-3-amine |
| | 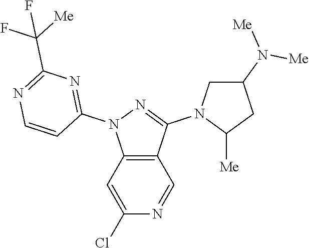 [SMILES: CC1CC(N(C)C)CN1c1nn(-c2ccnc(C(C)(F)F)n2)c2cc(Cl)ncc12] |
| | SM: 1-(6-chloro-1H-pyrazolo[4,3-c]pyridin-3-yl)-N,N,5- |
| | trimethylpyrrolidin-3-amine (Preparation 635) |
| | Prep-HPLC-I (22% to 42% MeCN). 20.2 mg, |
| | 67.0% yield as a yellow solid. |
| | ¹H NMR: (500 MHz, MeOD-d₄) δ ppm: |
| | 9.06 (d, J = 8.0 Hz, 1H), 8.84 (d, |
| | J = 5.5 Hz, 1H), 8.73 (s, 1H), 7.89-7.93 |
| | (m, 1H), 4.36-4.41 (m, 2H), 4.03- |
| | 4.11 (m, 1H), 3.03-3.31 (m, 6H), 2.58- |
| | 2.90 (m, 1H), 2.11 (d, J = 18.5 Hz, |
| | 3H), 1.56-1.57 (m, 2H), 1.30-1.43 (m, 3H). |
| 661 | 1-(6-chloro-1-(2-(1,1-difluoroethyl)pyrimidin- |
| | 4-yl)-1H-pyrazolo[4,3-c]pyridin- |
| | 3-yl)-4,4-difluoropyrrolidin-3-amine |
| | 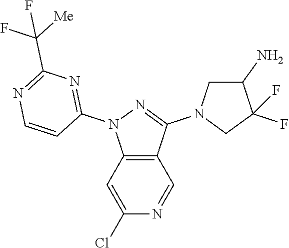 [SMILES: CC(F)(F)c1nccc(-n2nc(N3CC(N)C(F)(F)C3)c3cnc(Cl)cc32)n1] |
| | SM: 1-(6-chloro-1H-pyrazolo[4,3-c] |
| | pyridin-3-yl)-4,4-difluoropyrrolidin-3- |
| | amine hydrochloride (Preparation 609) |
| | 280 mg, 78.2% yield as a yellow solid. |
| | ¹H NMR (400 MHz, DMSO-d₆) δ |
| | ppm: 9.09 (s, 1H), 8.93 (d, J = 5.6 Hz, 1H), |
| | 8.56 (s, 1H), 7.83 (d, J = 6.0 Hz, |
| | 1H), 4.09-4.25 (m, 3H), 3.77-3.78 (m, 1H), 3.55-3.59 |
| | (m, 1H), 2.11 (t, J = 19.2 Hz, 3H). |
| 662 | 1-(6-chloro-1-(2-(1,1-difluoroethyl)pyrimidin-4-yl)- |
| | 1H-pyrazolo[4,3-c]pyridin-3-yl)-4, |
| | 4-difluoro-N-methylpyrrolidin-3-amine |
| | 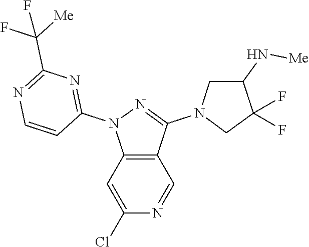 [SMILES: CNC1CN(c2nn(-c3ccnc(C(C)(F)F)n3)c3cc(Cl)ncc23)CC1(F)F] |
| | SM: 1-(6-chloro-1H-pyrazolo[4,3-c]pyridin-3-yl)- |
| | 4,4-difluoro-N-methylpyrrolidin- |
| | 3-amine hydrochloride (Preparation 607) |
| | 110 mg, 55.3% yield as a yellow solid. |
| | ¹H NMR: (400 MHz, CDCl₃) δ: ppm |
| | 8.81 (s, 1H), 8.78 (d, J = 5.6 Hz, 1H), 8.72 |
| | (s, 1H), 7.79 (d, J = 5.6 Hz, 1H), 4.05-4.16 |
| | (m, 3H), 3.64-3.66 (m, 2H), 2.64 (s, 3H), |
| | 2.09-2.18 (s, 3H). |
| 663 | 1-(6-chloro-1-(2-(1,1-difluoroethyl)pyrimidin- |
| | 4-yl)-1H-pyrazolo[4,3-c]pyridin-3-yl)- |
| | 4,4-difluoro-N,N-dimethylpyrrolidin-3-amine |
| | 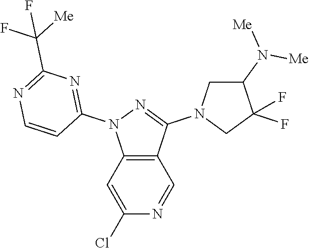 [SMILES: CN(C)C1CN(c2nn(-c3ccnc(C(C)(F)F)n3)c3cc(Cl)ncc23)CC1(F)F] |
| | SM: 1-(6-chloro-1H-pyrazolo[4,3-c] |
| | pyridin-3-yl)-4,4-difluoro-N,N- |
| | dimethylpyrrolidin-3-amine (Preparation 637) |
| | 100 mg, crude as a yellow solid. ¹H NMR |
| | (400 MHz, CDCl₃) δ: ppm 8.79 |
| | (d, J = 5.6 Hz, 1H), 8.73 (s, 1H), 8.02 (s, 1H), |
| | 7.81 (d, J = 5.6 Hz, 1H), 4.11- |
| | 4.24 (m, 3H), 3.90 (br s, 1H), 3.35-3.42 (m, 1H), |
| | 2.96 (s, 3H), 2.88 (s, 3H), 2.14 (t, J = 18.4 Hz, 3H). |

A-DMSO was the reaction solvent

### Preparations 664 to 680

The compounds in the following table were prepared from the appropriate 6-chloro-1H-pyrazolo[4,3-c]pyridine and 4-chloro-2-(1,1-difluoroethyl)-6-methylpyrimidine (Preparation 218) following a similar procedure to that described in Preparation 650.

| Preparation | |
|---|---|
| No | Name, Structure, Starting material (SM), Data |
| 664 | benzyl (1-(6-chloro-1-(2-(1,1-difluoroethyl)- |
| | 6-methylpyrimidin-4-yl)-1H- |
| | pyrazolo[4,3-c]pyridin- |
| | 3-yl)pyrrolidin-3-yl)(methyl)carbamate |
| | 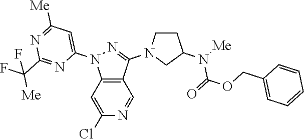 [SMILES: Cc1cc(-n2nc(N3CCC(N(C)C(=O)OCc4ccccc4)C3)c3cnc(Cl)cc32)nc(C(C)(F)F)n1] |
| | SM: benzyl (1-(6-chloro-1H-pyrazolo |
| | [4,3-c]pyridin-3-yl)pyrrolidin-3- |
| | yl)(methyl)carbamate hydrochloride |
| | (Preparation 623) |
| | 420 mg, 76.7% as yellow oil. ¹HNMR: |
| | (500 MHz, CDCl₃) δ ppm: 8.82 (s, |
| | 1H), 8.70 (s, 1H), 7.63 (s, 1H), 7.35-7.38 |
| | (m, 5H), 5.19 (s, 2H), 4.10-4.14 |
| | (m, 1H), 3.94-3.95 (m, 2H), 3.76-3.77 |
| | (m, 1H), 3.65-3.68 (m, 1H), 2.97 (s, |
| | 3H), 2.64 (s, 3H), 2.25-2.33 (m, 2H), 2.09-2.24 (m, 3H). |
| 665 | 1-(6-chloro-1-(2-(1,1-difluoroethyl)-6- |
| | methylpyrimidin-4-yl)-1H-pyrazolo[4,3-c] |
| | pyridin-3-yl)-N,N-dimethylazetidin-3-amine |
| | 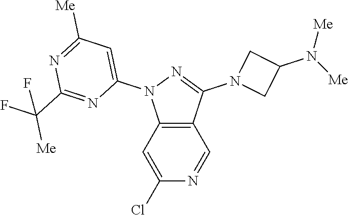 [SMILES: Cc1cc(-n2nc(N3CC(N(C)C)C3)c3cnc(Cl)cc32)nc(C(C)(F)F)n1] |
| | SM: 1-(6-chloro-1H-pyrazolo[4,3-c]pyridin-3-yl)-N,N- |
| | dimethylazetidin-3-amine (Preparation 638) |
| | 90 mg, 92.6% yield as a yellow solid. ¹H NMR (400 MHz, |
| | CDCl₃) δ: ppm 8.64 (s, 2H), 7.62 |
| | (s, 1H), 4.35-4.39 (m, 2H), 4.18 (br s, 2H), 3.44 (br s, |
| | 1H), 2.64 (s, 3H), 2.28 (s, 6H), 2.07-2.16 (m, 3H). |
| 666 | 1-(6-chloro-1-(2-(1,1-difluoroethyl)-6-methylpyrimidin- |
| | 4-yl)-1H-pyrazolo[4,3-c]pyridin-3-yl)- |
| | N,N,3-trimethylazetidin-3-amine |
| | 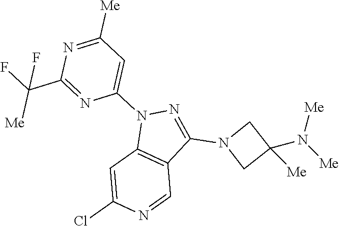 [SMILES: Cc1cc(-n2nc(N3CC(C)(N(C)C)C3)c3cnc(Cl)cc32)nc(C(C)(F)F)n1] |
| | SM: 1-(6-chloro-1H-pyrazolo[4,3-c]pyridin- |
| | 3-yl)-N,N,3-trimethylazetidin- |
| | 3-amine hydrochloride (Preparation 620) |
| | Prep-HPLC-D (24% to 44% MeCN). 110.0 mg, |
| | 34.6% yield as a yellow solid. ¹H NMR: (400 MHz, |
| | CDCl₃) δ ppm 8.68 (s, 1H), 8.66 (s, 1H), 7.64 (s, |
| | 1H), 4.82 (d, J = 8.4 Hz, 2H), 4.17 (d, J = |
| | 8.8 Hz, 2H), 2.80 (s, 6H), 2.65 (s, |
| | 3H), 2.12 (t, J = 18.4 Hz, 3H), 1.85 (s, 3H) |
| 667 | Cis-rac-1-(6-chloro-1-(2-(1,1-difluoroethyl)-6- |
| | methylpyrimidin-4-yl)-1H-pyrazolo[4,3-c] |
| | pyridin-3-yl)-N,N,4-trimethylpyrrolidin-3-amine |
| | 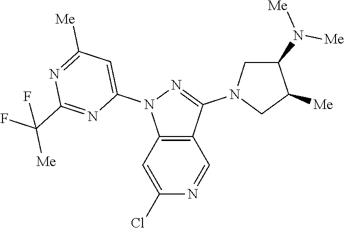 [SMILES: Cc1cc(-n2nc(N3C[C@@H](N(C)C)[C@@H](C)C3)c3cnc(Cl)cc32)nc(C(C)(F)F)n1] |
| | SM: cis-rac-1-(6-chloro-1H-pyrazolo[4,3- |
| | c]pyridin-3-yl)-N,N,4-trimethylpyrrolidin-3-amine |
| | (Preparation 636) |
| | 20 mg, 42.8% yield as a yellow solid. |
| | LCMS m/z = 436.3 [M + H]⁺. |
| 668 | 1-(6-chloro-1-(2-(1,1-difluoroethyl)-6-methylpyrimidin- |
| | 4-yl)-1H-pyrazolo[4,3-c] |
| | pyridin-3-yl)-3-(difluoromethyl)pyrrolidin-3-ol |
| | 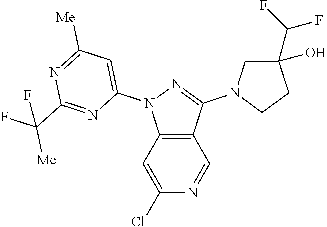 [SMILES: Cc1cc(-n2nc(N3CCC(O)(C(F)F)C3)c3cnc(Cl)cc32)nc(C(C)(F)F)n1] |
| | SM: 1-(6-chloro-1H-pyrazolo[4,3-c]pyridin-3-yl)-3- |
| | (difluoromethyl)pyrrolidin-3-ol hydrochloride |
| | (Preparation 610) |
| | 112.8 mg, 73.2% as yellow solid. ¹H NMR (500 MHz, |
| | CDCl₃) δ ppm: 8.83 (s, 1H), 8.69 (s, 1H), 7.63 |
| | (s, 1H), 5.95 (t, J = 55.5 Hz, 1H), 3.99-4.06 (m, |
| | 3H), 3.78-3.81 (m, 1H), 2.65 (s, 3H), 2.37-2.42 (m, 2H), |
| | 2.17-2.19 (m, 1H), 2.12 (d, J = 18.5 Hz, 3H). |
| 669 | 6-chloro-1-(2-(1,1-difluoroethyl)-6- |
| | methylpyrimidin-4-yl)-N-(2-methoxy-2- |
| | methylpropyl)-N-methyl-1H-pyrazolo[4,3-c] |
| | pyridin-3-amine |
| | 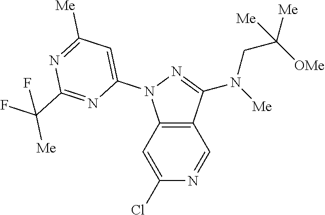 [SMILES: COC(C)(C)CN(C)c1nn(-c2cc(C)nc(C(C)(F)F)n2)c2cc(Cl)ncc12] |
| | SM: 6-chloro-N-(2-methoxy-2-methylpropyl)-N-methyl- |
| | 1H-pyrazolo[4,3-c]pyridin-3-amine hydrochloride |
| | (Preparation 611) |
| | 85 mg, 53.8% yield as a yellow solid. |
| | ¹H NMR: (500 MHz, CDCl₃) δ ppm |
| | 9.01 (s 1H), 8.72 (s, 1H), 7.59 (s, 1H), |
| | 3.69 (s, 2H), 3.41 (s, 3H), 3.25 (s, |
| | 3H), 2.64 (s, 3H), 2.12 (t, J = 18.5 Hz, 3H), 1.26 (s, 6H). |
| 670 | 6-chloro-1-(2-(1,1-difluoroethyl)-6-methylpyrimidin- |
| | 4-yl)-N-(2-methoxyethyl)-N- |
| | methyl-1H-pyrazolo[4,3-c]pyridin-3-amine |
| | 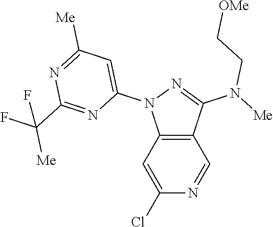 [SMILES: COCCN(C)c1nn(-c2cc(C)nc(C(C)(F)F)n2)c2cc(Cl)ncc12] |
| | SM: 6-chloro-N-(2-methoxyethyl)-N- |
| | methyl-1H-pyrazolo[4,3-c]pyridin-3- |
| | amine (Preparation 612) |
| | 95 mg, 52.4% yield) as a yellow solid. ¹H NMR (500 |
| | MHz, CDCl₃) δ ppm: 8.95 (s, 1H), 8.72 (s, 1H), 7.62 |
| | (s, 1H), 3.82 (t, J = 5.5 Hz, 2H), 3.72 (t, J = 5.5 |
| | Hz, 2H), 3.39 (s, 3H), 3.35 (s, 3H), 2.64 (s, 3H), |
| | 2.12 (t, J = 18.5 Hz, 3H). |
| 671 | N-benzyl-6-chloro-1-(2-(1,1-difluoroethyl)-6- |
| | methylpyrimidin-4-yl)-N- |
| | methyl-1H-pyrazolo[4,3-c]pyridin-3-amine |
| | 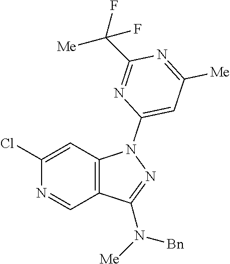 [SMILES: Cc1cc(-n2nc(N(C)Cc3ccccc3)c3cnc(Cl)cc32)nc(C(C)(F)F)n1] |
| | SM: N-benzyl-6-chloro-N-methyl-1H- |
| | pyrazolo[4,3-c]pyridin-3-amine |
| | (Preparation 613) |
| | 103 mg, 74.3% yield as a white solid. |
| | LCMS m/z = 429.1[M + H]⁺ |
| 672 | N-benzyl-6-chloro-1-(2-(1,1-difluoroethyl)- |
| | 6-methylpyrimidin-4-yl)-1H- |
| | pyrazolo[4,3-c]pyridin-3-amine |
| | 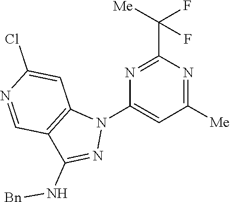 [SMILES: Cc1cc(-n2nc(NCc3ccccc3)c3cnc(Cl)cc32)nc(C(C)(F)F)n1] |
| | SM: N-benzyl-6-chloro-1H-pyrazolo[4,3-c]pyridin-3- |
| | amine (Preparation 615) |
| | 350 mg, 14.6% as a white solid. |
| | LCMS m/z = 415.1[M + H]⁺ |
| 673 | N-benzyl-6-chloro-1-(2-(1,1-difluoroethyl)- |
| | 6-methylpyrimidin-4-yl)-N- |
| | ethyl-1H-pyrazolo[4,3-c]pyridin-3-amine |
| | 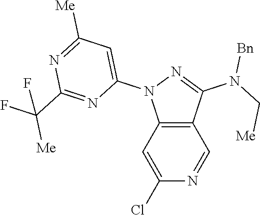 [SMILES: CCN(Cc1ccccc1)c1nn(-c2cc(C)nc(C(C)(F)F)n2)c2cc(Cl)ncc12] |
| | SM: N-benzyl-6-chloro-N-ethyl-1H-pyrazolo[4,3-c] |
| | pyridin-3-amine hydrochloride (Preparation 614) |
| | 315 mg, 57.5% yield as a yellow solid. |
| | LCMS m/z = 443.2 [M + H]⁺ |
| 674 | 6-chloro-1-(2-(1,1-difluoroethyl)-6-methylpyrimidin- |
| | 4-yl)-N-methyl-N-(1-methylpyrrolidin- |
| | 3-yl)-1H-pyrazolo[4,3-c]pyridin-3-amine |
| | 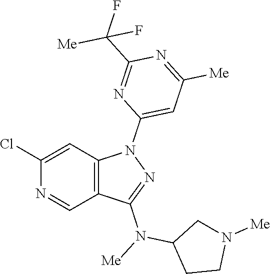 [SMILES: Cc1cc(-n2nc(N(C)C3CCN(C)C3)c3cnc(Cl)cc32)nc(C(C)(F)F)n1] |
| | SM: 6-chloro-N-methyl-N-(1-methylpyrrolidin- |
| | 3-yl)-1H-pyrazolo[4,3- |
| | c]pyridin-3-amine (Preparation 631) |
| | 72.6 mg, 41.1% yield as yellow solid. |
| | ¹H NMR (400 MHz, CDCl₃) δ ppm: |
| | 8.90 (s, 1H), 8.71 (s, 1H), 7.63 (s, 1H), 4.96- |
| | 5.03 (m, 1H), 3.30 (s, 3H), 2.84-2.86 |
| | (m, 3H), 2.65 (s, 3H), 2.33-2.45 (m, 5H), 2.12 (d, |
| | J = 18.8 Hz, 3H), 2.03-2.07 (m, 1H). |
| 675 | 1-(6-chloro-1-(2-(1,1-difluoroethyl)-6- |
| | methylpyrimidin-4-yl)-1H-pyrazolo[4,3-c] |
| | pyridin-3-yl)-N,N-dimethylpiperidin-3-amine |
| | 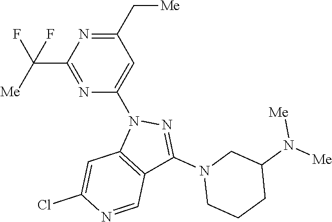 [SMILES: CCc1cc(-n2nc(N3CCCC(N(C)C)C3)c3cnc(Cl)cc32)nc(C(C)(F)F)n1] |
| | SM: 1-(6-chloro-1H-pyrazolo[4,3-c]pyridin- |
| | 3-yl)-N,N-dimethylpiperidin-3- |
| | amine (Preparation 643) |
| | Prep-HPLC-C (49% to 79% MeCN) |
| | 30 mg, 48.1% as a brown solid. |
| | LCMS m/z = 436.1[M + H]⁺ |
| 676 | 1-(6-chloro-1-(2-(1,1-difluoroethyl)- |
| | 6-methylpyrimidin-4-yl)-1H-pyrazolo[4,3-c] |
| | pyridin-3-yl)-N,N-dimethylpiperidin-4-amine |
| | 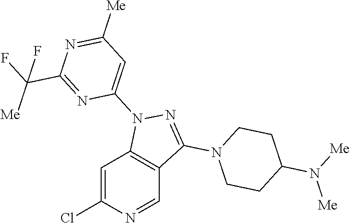 [SMILES: Cc1cc(-n2nc(N3CCC(N(C)C)CC3)c3cnc(Cl)cc32)nc(C(C)(F)F)n1] |
| | SM: 1-(6-chloro-1H-pyrazolo[4,3-c]pyridin- |
| | 3-yl)-N,N-dimethylpiperidin-4- |
| | amine (Preparation 617) |
| | Prep-HPLC-U (18% to 38% MeCN). 100 mg, 42.8% |
| | yield as a yellow solid. |
| | ¹H NMR: (400 MHz, CDCl₃) δ: ppm 8.86 (s, 1H), |
| | 8.74 (s, 1H), 7.64 (s, 1H), 4.29 (d, J = 13.6 Hz, |
| | 2H), 3.15 (t, J = 12.0 Hz, 2H), 2.95 (t, J = 11.6 Hz, 1H), |
| | 2.66 (s, 3H), 2.57 (s, 6H), 2.04-2.12 (m, 3H), 1.85-1.91 |
| | (m, 2H), 1.24-1.28 (m, 2H). |
| 677^{A} | 6-chloro-1-(2-(1,1-difluoroethyl)-6-methylpyrimidin- |
| | 4-yl)-3-(7-methyl-2,7-diazaspiro[4.4] |
| | nonan-2-yl)-1H-pyrazolo[4,3-c]pyridine |
| | 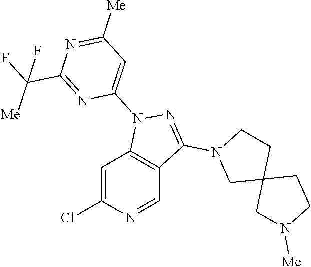 [SMILES: Cc1cc(-n2nc(N3CCC4(CCN(C)C4)C3)c3cnc(Cl)cc32)nc(C(C)(F)F)n1] |
| | SM: 6-chloro-3-(7-methyl-2,7-diazaspiro[4.4]nonan-2-yl)- |
| | 1H-pyrazolo[4,3-c]pyridine (Preparation 639) |
| | Prep-HPLC-C (60% to 90% MeCN) |
| | 70 mg, 38% as white solid. LCMS m/z = 448.1[M + H]⁺ |
| 678 | 6-chloro-1-(2-(1,1-difluoroethyl)-6-methylpyrimidin- |
| | 4-yl)-3-(7-methyl-2,7- |
| | diazaspiro[3.5]nonan-2-yl)-1H-pyrazolo[4,3-c]pyridine |
| | 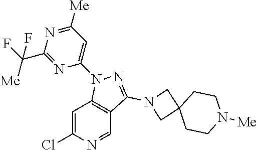 [SMILES: Cc1cc(-n2nc(N3CC4(CCN(C)CC4)C3)c3cnc(Cl)cc32)nc(C(C)(F)F)n1] |
| | SM: 6-chloro-3-(7-methyl-2,7-diazaspiro[3.5]nonan-2-yl)- |
| | 1H-pyrazolo[4,3-c]pyridine (Preparation 640) |
| | prep-HPLC-L (62% to 92% MeCN) |
| | 34 mg, 36.9% yield as a white solid. ¹H NMR: |
| | (500 MHz, CDCl₃) δ ppm: 8.65 (s, 1H), 8.64 (s, 1H), |
| | 7.62 (s, 1H), 4.06 (s, 4H), 2.64 (s, 3H), 2.35 (br s, |
| | 4H), 2.12 (t, J = 18.5 Hz, 3H), 1.98-2.00 (m, 3H), |
| | 1.56 (br s, 4H). |
| 679 | 6-chloro-1-(2-(1,1-difluoroethyl)-6-methylpyrimidin- |
| | 4-yl)-3-(6-methyl-2,6- |
| | diazaspiro[3.4]octan-2-yl)-1H-pyrazolo[4,3-c]pyridine |
| | 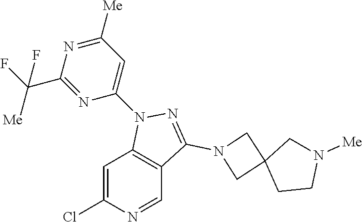 [SMILES: Cc1cc(-n2nc(N3CC4(CCN(C)C4)C3)c3cnc(Cl)cc32)nc(C(C)(F)F)n1] |
| | SM: 6-chloro-3-(6-methyl-2,6-diazaspiro[3.4]octan-2-yl)- |
| | 1H-pyrazolo[4,3-c]pyridine (Preparation 642) |
| | Prep-HPLC-C (40% to 70% MeCN). |
| | 25 mg, 64.0% as a white solid. |
| | LCMS m/z = 434.1[M + H]⁺ |
| 680 | 9-(6-chloro-1-(2-(1,1-difluoroethyl)-6-methylpyrimidin- |
| | 4-yl)-1H-pyrazolo[4,3-c]pyridin-3-yl)-2- |
| | methyl-6-oxa-2,9-diazaspiro[4.5]decane |
| | 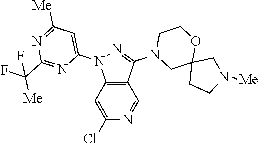 [SMILES: Cc1cc(-n2nc(N3CCOC4(CCN(C)C4)C3)c3cnc(Cl)cc32)nc(C(C)(F)F)n1] |
| | SM: 9-(6-chloro-1H-pyrazolo[4,3-c]pyridin- |
| | 3-yl)-2-methyl-6-oxa-2,9- |
| | diazaspiro[4.5]decane (Preparation 641) |
| | 50 mg, 30.2% yield) as a yellow solid. |
| | LCMS m/z = 464.1[M + H]⁺ |

A—the reaction was performed at rt.

### Preparations 681 to 689

The compounds in the following table were prepared from (S)-1-(6-chloro-1H-pyrazolo[4,3-c]pyridin-3-yl)-N,N-dimethylpyrrolidin-3-amine (Preparation 627) and the appropriate amine, following a similar procedure to that described in Preparation 650.

| Preparation | |
|---|---|
| No | Name, Structure, Starting amine, Data |
| 681 | (S)-1-(6-chloro-1-(6-chloro-2-(1,1-difluoroethyl) |
| | pyrimidin-4-yl)-1H-pyrazolo[4,3-c] |
| | pyridin-3-yl)-N,N-dimethylpyrrolidin-3-amine |
| | 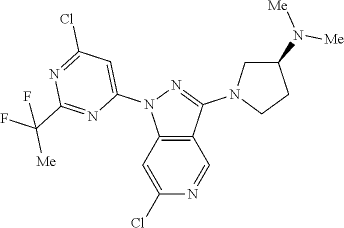 [SMILES: CN(C)[C@H]1CCN(c2nn(-c3cc(Cl)nc(C(C)(F)F)n3)c3cc(Cl)ncc23)C1] |
| | SM: 4,6-dichloro-2-(1,1-difluoroethyl)pyrimidine |
| | (Preparation 189) |
| | 385.5 mg, 69.8% yield as a yellow solid. |
| | LCMS m/z = 442.1[M + H]⁺ |
| 682 | (S)-1-(6-chloro-1-(2-(1,1-difluoroethyl)-6- |
| | isopropoxypyrimidin-4-yl)-1H-pyrazolo |
| | [4,3-c]pyridin-3-yl)-N,N-dimethylpyrrolidin-3-amine |
| | 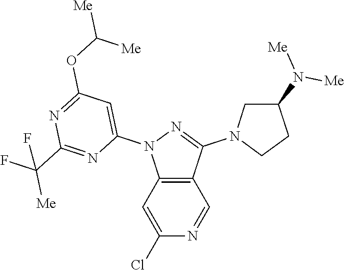 [SMILES: CC(C)Oc1cc(-n2nc(N3CC[C@H](N(C)C)C3)c3cnc(Cl)cc32)nc(C(C)(F)F)n1] |
| | SM: 4-chloro-2-(1,1-difluoroethyl)-6- |
| | isopropoxypyrimidine (Preparation 471) |
| | 113 mg, 82.0% yield as a yellow solid. |
| | LCMS m/z = 467.8 [M + H]⁺ |
| 683 | (S)-1-(6-chloro-1-(2-(1,1-difluoroethyl)-6- |
| | ethylpyrimidin-4-yl)-1H-pyrazolo[4,3-c] |
| | pyridin-3-yl)-N,N-dimethylpyrrolidin-3-amine |
| | 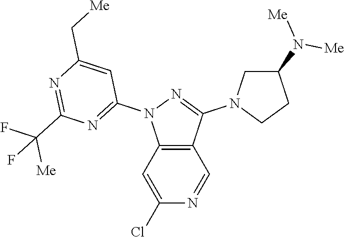 [SMILES: CCc1cc(-n2nc(N3CC[C@H](N(C)C)C3)c3cnc(Cl)cc32)nc(C(C)(F)F)n1] |
| | SM: 4-chloro-2-(1,1-difluoroethyl)-6-ethylpyrimidine |
| | (Preparation 390) |
| | 93.5 mg, 57% yield as a yellow solid. ¹H NMR: |
| | (400 MHz, CDCl₃) δ ppm: |
| | 8.84 (s, 1H), 8.70 (s, 1H), 7.63 (s, 1H), |
| | 3.93-3.98 (m, 2H), 3.76-3.79 (m, |
| | 1H), 3.56-3.57 (m, 1H), 2.87-2.93 (m, 3H), |
| | 2.39 (s, 6H), 2.31-2.33 (m, 1H), |
| | 2.08-2.17 (m, 4H), 1.38 (t, J = 7.6 Hz, 3H). |
| 684 | (S)-1-(6-chloro-1-(6-cyclopropyl-2-(1,1-difluoroethyl) |
| | pyrimidin-4-yl)-1H-pyrazolo[4,3-c] |
| | pyridin-3-yl)-N,N-dimethylpyrrolidin-3-amine |
| | 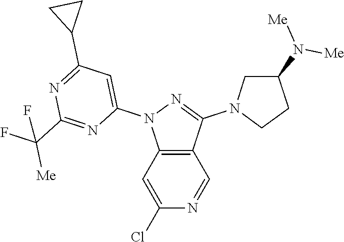 [SMILES: CN(C)[C@H]1CCN(c2nn(-c3cc(C4CC4)nc(C(C)(F)F)n3)c3cc(Cl)ncc23)C1] |
| | SM: 4-chloro-6-cyclopropyl-2-(1,1-difluoroethyl) |
| | pyrimidine (Preparation 415) |
| | 160.3 mg, crude as a yellow solid. |
| | LCMS m/z = 448.2 [M + H]⁺ |
| 685 | (S)-1-(6-chloro-1-(2-(1,1-difluoroethyl)-6-(2- |
| | methoxyethoxy)pyrimidin-4-yl)-1H-pyrazolo[4,3-c] |
| | pyridin-3-yl)-N,N-dimethylpyrrolidin-3-amine |
| | 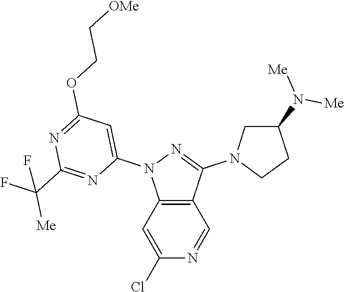 [SMILES: COCCOc1cc(-n2nc(N3CC[C@H](N(C)C)C3)c3cnc(Cl)cc32)nc(C(C)(F)F)n1] |
| | SM: 4-chloro-2-(1,1-difluoroethyl)-6-(2- |
| | methoxyethoxy)pyrimidine (Preparation 474) |
| | 220 mg, 74.6% yield as a yellow solid. |
| | LCMS m/z = 482.2 [M + H]⁺ |
| 686 | (S)-1-(6-chloro-1-(2-(1,1-difluoroethyl)-6-((1r,3S)-3- |
| | methoxycyclobutoxy)pyrimidin-4-yl)-1H-pyrazolo[4,3-c] |
| | pyridin-3-yl)-N,N-dimethylpyrrolidin-3-amine |
| | 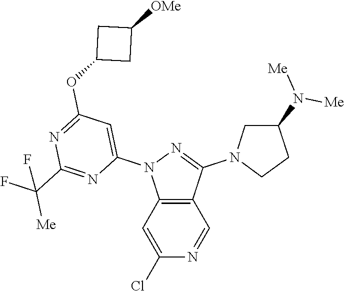 [SMILES: CO[C@H]1C[C@H](Oc2cc(-n3nc(N4CC[C@H](N(C)C)C4)c4cnc(Cl)cc43)nc(C(C)(F)F)n2)C1] |
| | SM: 4-chloro-2-(1,1-difluoroethyl)-6-((1r,3r)-3- |
| | methoxycyclobutoxy)pyrimidine (Preparation 475) |
| | 70 mg, 36.7% yield as a white solid. |
| | LCMS m/z = 507.4 [M + H]⁺ |
| 687 | (S)-1-(6-chloro-1-(6-cyclopropoxy-2-(1,1- |
| | difluoroethyl)pyrimidin-4-yl)-1H-pyrazolo[4,3-c] |
| | pyridin-3-yl)-N,N-dimethylpyrrolidin-3-amine |
| | 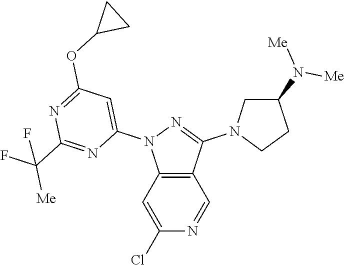 [SMILES: CN(C)[C@H]1CCN(c2nn(-c3cc(OC4CC4)nc(C(C)(F)F)n3)c3cc(Cl)ncc23)C1] |
| | SM: 4-chloro-6-cyclopropoxy-2-(1,1-difluoroethyl) |
| | pyrimidine (Preparation 473) |
| | 65 mg, 25.9% as yellow solid. |
| | LCMS m/z = 464.1[M + H]⁺ |
| 688 | (S)-1-(6-chloro-1-(6-(1,1-difluoroethyl)-4-((1s,3R)-3- |
| | methoxycyclobutoxy)pyridin-2-yl)-1H-pyrazolo[4,3-c] |
| | pyridin-3-yl)-N,N-dimethylpyrrolidin-3-amine |
| | 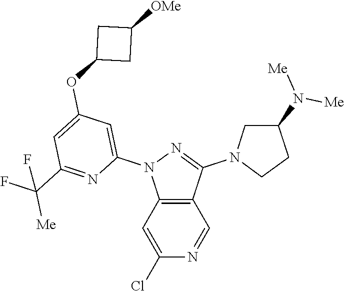 [SMILES: CO[C@H]1C[C@@H](Oc2cc(-n3nc(N4CC[C@H](N(C)C)C4)c4cnc(Cl)cc43)nc(C(C)(F)F)c2)C1] |
| | SM: 2-chloro-6-(1,1-difluoroethyl)-4-((1s,3s)-3- |
| | methoxycyclobutoxy)pyridine (Preparation 480) |
| | 100 mg, 23.8% yield as a white solid. |
| | LCMS m/z = 507.2 [M + H]⁺ |
| 689 | (S)-1-(6-chloro-1-(2-(1,1-difluoroethyl)-6-ethoxypyrimidin- |
| | 4-yl)-1H-pyrazolo[4,3-c] |
| | pyridin-3-yl)-N,N-dimethylpyrrolidin-3-amine |
| | 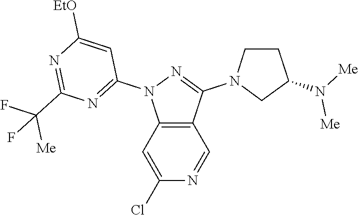 [SMILES: CCOc1cc(-n2nc(N3CC[C@H](N(C)C)C3)c3cnc(Cl)cc32)nc(C(C)(F)F)n1] |
| | SM: 4-chloro-2-(1,1-difluoroethyl)-6-ethoxypyrimidine |
| | (Preparation 472) |
| | 96 mg, 47.3% as yellow solid. ¹H NMR: (500 MHz, |
| | CDCl₃) δ ppm: 8.82 (s, 1H), 8.67 (s, 1H), |
| | 7.11 (s, 1H), 4.50-4.54 (m, 2H), 3.91-3.95 (m, 2H), 3.72- |
| | 3.78 (m, 1H), 3.52-3.55 (m, 1H), 2.96 (br s, |
| | 1H), 2.37 (s, 6H), 2.30-2.32 (m, |
| | 1H), 2.04-2.13 (m, 4H), 1.44 (t, J = 7.0 Hz, 3H). |

### Preparations 690 to 691

The compounds in the following table were prepared from the appropriate 6-chloro-1H-pyrazolo[4,3-c]pyridine and amine, following a similar procedure to that described in Preparation 650.

| Preparation | |
|---|---|
| No | Name, Structure, Starting materials (SM) and Data |
| 690 | (R)-1-(6-chloro-1-(6-(1,1-difluoroethyl) |
| | pyridin-2-yl)-1H-pyrazolo[4,3- |
| | c]pyridin-3-yl)-N,N-dimethylpyrrolidin-3-amine |
| | 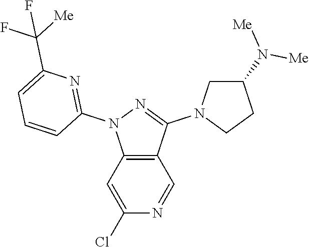 [SMILES: CN(C)[C@@H]1CCN(c2nn(-c3cccc(C(C)(F)F)n3)c3cc(Cl)ncc23)C1] |
| | SM: (R)-1-(6-chloro-1H-pyrazolo[4,3-c]pyridin- |
| | 3-yl)-N,N-dimethylpyrrolidin-3-amine |
| | (Preparation 633) and 2-bromo-6-(1,1- |
| | difluoroethyl)pyridine |
| | 240 mg, 38.2% yield as a yellow solid. |
| | ¹H NMR (400 MHz, CDCl₃) δ: ppm |
| | 8.83 (s, 1H), 8.58 (s, 1H), 7.87-7.96 (m, |
| | 2H), 7.42 (d, J = 7.6 Hz, 1H), 3.92- |
| | 3.97 (m, 2H), 3.75-3.78 (m, 1H), 3.49- |
| | 3.50 (m, 1H), 3.44-3.48 (m, 2H), 2.39 |
| | (s, 6H), 2.29-2.33 (m, 1H), 2.12 (t, J = 18.4 Hz, 3H). |
| 691 | 1-(6-chloro-1-(2-(1,1-difluoroethyl)-6- |
| | methoxypyrimidin-4-yl)-1H-pyrazolo[4,3-c] |
| | pyridin-3-yl)-N,N-dimethylazetidin-3-amine |
| | 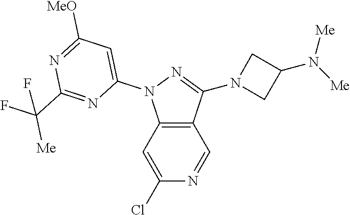 [SMILES: COc1cc(-n2nc(N3CC(N(C)C)C3)c3cnc(Cl)cc32)nc(C(C)(F)F)n1] |
| | SM: 1-(6-chloro-1H-pyrazolo[4,3-c]pyridin- |
| | 3-yl)-N,N-dimethylazetidin-3-amine |
| | (Preparation 638) and 4-chloro-2-(1,1-difluoroethyl)-6- |
| | methoxypyrimidine (Preparation 190) |
| | 80 mg, 95% yield as yellow solid. |
| | LCMS m/z = 424.0 [M + H]⁺ |

### Preparation 692: 1-(6-chloro-1-(2-(1,1-difluoroethyl)pyrimidin-4-yl)-1H-pyrazolo[4,3-c]pyridin-3-yl)-4-methylpyrrolidin-3-ol

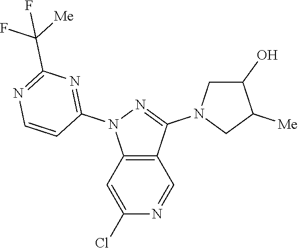 [SMILES: CC1CN(c2nn(-c3ccnc(C(C)(F)F)n3)c3cc(Cl)ncc23)CC1O]

To a solution of 1-(6-chloro-1H-pyrazolo[4,3-c]pyridin-3-yl)-4-methylpyrrolidin-3-ol (35.0 mg, 79.9 μmol), 4-chloro-2-(1,1-difluoroethyl)pyrimidine (35.4 mg, 0.198 mmol) and K₃PO₄ (58.8 mg, 0.277 mmol) in dioxane (2.0 mL) was added Pd₂(dba)₃ (18.2 mg, 19.8 μmol) and Xantphos (11.5 mg, 19.8 μmol) and the reaction mixture was stirred at 70° C. under N₂ for 1 h. The mixture was quenched with H₂O (50 mL) and extracted with EtOAc (20 mL×3). The combined organic layers were washed with brine (20 mL), dried over Na₂SO₄, filtered and concentrated under vacuum to give the crude, which was purified on silica gel column chromatography (PE/EtOAc=1/0 to 1/1) to give 1-(6-chloro-1-(2-(1,1-difluoroethyl)pyrimidin-4-yl)-1H-pyrazolo[4,3-c]pyridin-3-yl)-4-methylpyrrolidin-3-ol (70.0 mg, 89.6% yield) as a white solid. LCMS m/z=395.1 [M+H]⁺

### Preparation 693: 3-(6-chloro-1-(2-(1,1-difluoroethyl)-6-methylpyrimidin-4-yl)-1H-pyrazolo[4,3-c]pyridin-3-yl)-6-ethyl-3,6-diazabicyclo[3.1.1]heptane

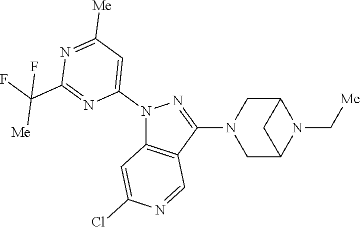 [SMILES: CCN1C2CC1CN(c1nn(-c3cc(C)nc(C(C)(F)F)n3)c3cc(Cl)ncc13)C2]

3-(6-Chloro-1-(2-(1,1-difluoroethyl)-6-methylpyrimidin-4-yl)-1H-pyrazolo[4,3-c]pyridin-3-yl)-6-ethyl-3,6-diazabicyclo[3.1.1]heptane was obtained as a yellow solid, 60 mg, 54.9% yield, from 3-(6-chloro-1H-pyrazolo[4,3-c]pyridin-3-yl)-6-ethyl-3,6-diazabicyclo[3.1.1]heptane (Preparation 644) and 4-chloro-2-(1,1-difluoroethyl)-6-methylpyrimidine (Preparation 218), following the procedure described in Preparation 692. LCMS m/z=434.1 [M+H]⁺

### Preparation 694: tert-butyl 3-(6-chloro-1-(2-(1,1-difluoroethyl)-6-ethylpyrimidin-4-yl)-1H-pyrazolo[4,3-c]pyridin-3-yl)-3,6-diazabicyclo[3.1.1]heptane-6-carboxylate

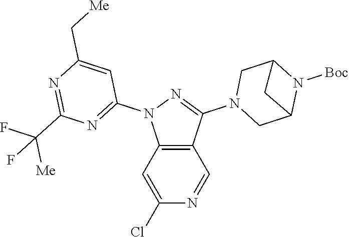 [SMILES: CCc1cc(-n2nc(N3CC4CC(C3)N4C(=O)OC(C)(C)C)c3cnc(Cl)cc32)nc(C(C)(F)F)n1]

tert-Butyl 3-(6-chloro-1-(2-(1,1-difluoroethyl)-6-ethylpyrimidin-4-yl)-1H-pyrazolo[4,3-c]pyridin-3-yl)-3,6-diazabicyclo[3.1.1]heptane-6-carboxylate was obtained as a white solid, 350 mg, 27.1%, from tert-butyl 3,6-diazabicyclo[3.1.1]heptane-6-carboxylate and 3-bromo-6-chloro-1-(2-(1,1-difluoroethyl)-6-ethylpyrimidin-4-yl)-1H-pyrazolo[4,3-c]pyridine (Preparation 646) following the procedure described in Preparation 693. ¹H NMR (500 MHz, CDCl₃) δ ppm:8.95 (s, 1H), 8.77 (m, 1H), 7.66 (s, 1H), 4.37-5.38 (m, 3H), 3.77 (d, J=10.0 Hz, 2H), 2.91 (m, 2H), 2.74-2.80 (m, 1H), 2.13 (t, J=18.5 Hz, 3H), 1.63 (d, J=8.5 Hz, 2H), 1.35-1.41 (m, 12H).

### Preparation 695: 3-(6-chloro-1-(2-(1,1-difluoroethyl)-6-ethylpyrimidin-4-yl)-1H-pyrazolo[4,3-c]pyridin-3-yl)-3,6-diazabicyclo[3.1.1]heptane hydrochloride

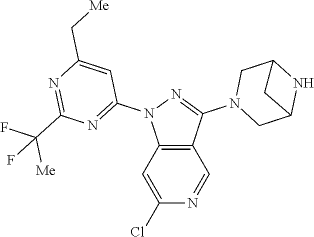 [SMILES: CCc1cc(-n2nc(N3CC4CC(C3)N4)c3cnc(Cl)cc32)nc(C(C)(F)F)n1]

To a solution of tert-butyl 3-(6-chloro-1-(2-(1,1-difluoroethyl)-6-ethylpyrimidin-4-yl)-1H-pyrazolo[4,3-c]pyridin-3-yl)-3,6-diazabicyclo[3.1.1]heptane-6-carboxylate (Preparation 694, 320 mg, 0.62 mmol) in DCM (3 mL) was added HCl/dioxane (4 M, 0.3 mL) and the mixture was stirred at 25° C. for 5 h. The mixture was concentrated under reduced pressure to give 3-(6-chloro-1-(2-(1,1-difluoroethyl)-6-ethylpyrimidin-4-yl)-1H-pyrazolo[4,3-c]pyridin-3-yl)-3,6-diazabicyclo[3.1.1]heptane hydrochloride (300 mg, crude) as yellow solid. LCMS m/z=420.2 [M+H]⁺

### Preparation 696: 3-(6-chloro-1-(2-(1,1-difluoroethyl)-6-ethylpyrimidin-4-yl)-1H-pyrazolo[4,3-c]pyridin-3-yl)-6-methyl-3,6-diazabicyclo[3.1.1]heptane

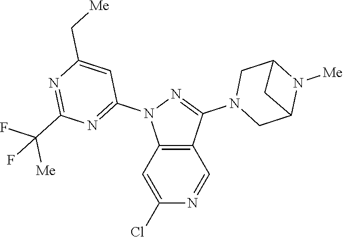 [SMILES: CCc1cc(-n2nc(N3CC4CC(C3)N4C)c3cnc(Cl)cc32)nc(C(C)(F)F)n1]

To a solution of 3-(6-chloro-1-(2-(1,1-difluoroethyl)-6-ethylpyrimidin-4-yl)-1H-pyrazolo[4,3-c]pyridin-3-yl)-3,6-diazabicyclo[3.1.1]heptane hydrochloride (Preparation 695, 300 mg, 0.66 mmol, HCl) in MeOH (10 mL) was added NaBH₃CN (206.6 mg, 3.29 mmol), (CH₂O)n (1.58 g, 1.31 mmol, 1.79 mL) and the mixture was stirred at 25° C. for 3 h. The mixture was filtered and concentrated to give a residue which was purified on silica gel column chromatography (DCM/MeOH=10/1) to give 3-(6-chloro-1-(2-(1,1-difluoroethyl)-6-ethylpyrimidin-4-yl)-1H-pyrazolo[4,3-c]pyridin-3-yl)-6-methyl-3,6-diazabicyclo[3.1.1]heptane (110 mg, 38.6% yield) as a white solid. ¹H NMR (400 MHz, DMSO-d₆) δ ppm: 9.20 (s, 1H), 8.62 (s, 1H), 7.74 (s, 1H), 3.99 (d, J=12.0 Hz, 2H), 3.75-3.82 (m, 2H), 3.64 (d, J=3.2 Hz, 2H), 2.88-2.89 (m, 2H), 2.56-2.58 (m, 1H) 2.05-2.17 (m, 6H), 1.64-1.66 (m, 1H), 1.29 (t, J=7.6 Hz, 3H), 1.23 (s, 1H).

### Preparation 697: 3-(6-chloro-1-(2-(1,1-difluoroethyl)-6-ethylpyrimidin-4-yl)-1H-pyrazolo[4,3-c]pyridin-3-yl)-6-ethyl-3,6-diazabicyclo[3.1.1]heptane

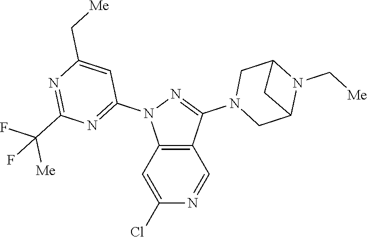 [SMILES: CCc1cc(-n2nc(N3CC4CC(C3)N4CC)c3cnc(Cl)cc32)nc(C(C)(F)F)n1]

3-(6-Chloro-1-(2-(1,1-difluoroethyl)-6-ethylpyrimidin-4-yl)-1H-pyrazolo[4,3-c]pyridin-3-yl)-6-ethyl-3,6-diazabicyclo[3.1.1]heptane was obtained as a yellow solid, 300 mg, 61.1%, from 3-(6-chloro-1-(2-(1,1-difluoroethyl)-6-ethylpyrimidin-4-yl)-1H-pyrazolo[4,3-c]pyridin-3-yl)-3,6-diazabicyclo[3.1.1]heptane hydrochloride (Preparation 695) and CH₃CHO, following a similar procedure to that described in Preparation 696. ¹H NMR (400 MHz, MeOD-d₄) δ ppm: 9.05 (s, 1H), 8.70 (s, 1H), 7.73 (s, 1H), 4.06-4.10 (m, 2H), 3.85-3.90 (m, 4H), 2.86-2.92 (m, 2H), 2.56-2.64 (m, 2H), 2.09 (t, J=18.4 Hz, 3H), 2.01 (s, 1H), 1.86-1.81 (m, 1H), 1.36 (t, J=7.6 Hz, 3H), 1.10 (t, J=7.2 Hz, 3H)

### Preparation 698: (S)-1-(6-chloro-1-(2-(1,1-difluoroethyl)-6-methoxypyrimidin-4-yl)-1H-pyrazolo[4,3-c]pyridin-3-yl)-N,N-dimethylpyrrolidin-3-amine

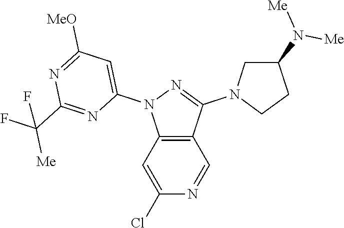 [SMILES: COc1cc(-n2nc(N3CC[C@H](N(C)C)C3)c3cnc(Cl)cc32)nc(C(C)(F)F)n1]

To a solution of (S)-1-(6-chloro-1-(6-chloro-2-(1,1-difluoroethyl)pyrimidin-4-yl)-1H-pyrazolo[4,3-c]pyridin-3-yl)-N,N-dimethylpyrrolidin-3-amine (Preparation 681, 200 mg, 0.452 mmol) in MeOH (5.0 mL) was added NaOMe (29.3 mg, 0.543 mmol) and the reaction mixture was stirred at 60° C. for 48 h. The mixture was concentrated, the residue diluted with water (15 mL) and extracted with DCM (20 mL×3). The combined organic phase was washed with brine (20 mL), dried over anhydrous Na₂SO₄, filtered and concentrated to give (S)-1-(6-chloro-1-(2-(1,1-difluoroethyl)-6-methoxypyrimidin-4-yl)-1H-pyrazolo[4,3-c]pyridin-3-yl)-N,N-dimethylpyrrolidin-3-amine (178 mg, 89.9% yield) as a yellow solid. LCMS m/z=438.1 [M+H]⁺

### Preparation 699: (S)-1-(6-chloro-1-(6-cyclobutoxy-2-(1,1-difluoroethyl)pyrimidin-4-yl)-1H-pyrazolo[4,3-c]pyridin-3-yl)-N,N-dimethylpyrrolidin-3-amine

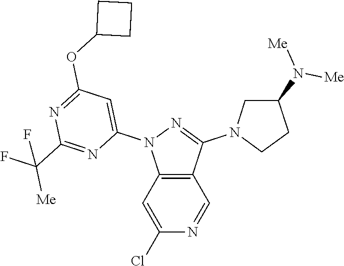 [SMILES: CN(C)[C@H]1CCN(c2nn(-c3cc(OC4CCC4)nc(C(C)(F)F)n3)c3cc(Cl)ncc23)C1]

(S)-1-(6-Chloro-1-(6-cyclobutoxy-2-(1,1-difluoroethyl)pyrimidin-4-yl)-1H-pyrazolo[4,3-c]pyridin-3-yl)-N,N-dimethylpyrrolidin-3-amine was obtained as a white solid, 110 mg, crude from cyclobutanol and (S)-1-(6-chloro-1-(6-chloro-2-(1,1-difluoroethyl)pyrimidin-4-yl)-1H-pyrazolo[4,3-c]pyridin-3-yl)-N,N-dimethylpyrrolidin-3-amine (Preparation 681) following a similar procedure to that described in Preparation 473. LCMS m/z=478.2 [M+H]⁺

### Preparation 700: 1-(6-chloro-1-(2-(1,1-difluoroethyl)-6-ethylpyrimidin-4-yl)-1H-pyrazolo[4,3-c]pyridin-3-yl)-N,N-dimethylpyrrolidin-3-amine

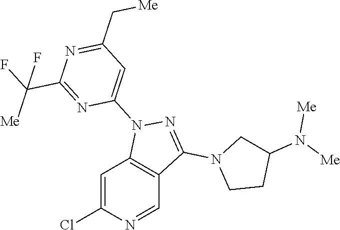 [SMILES: CCc1cc(-n2nc(N3CCC(N(C)C)C3)c3cnc(Cl)cc32)nc(C(C)(F)F)n1]

To a solution of 3-bromo-6-chloro-1-(2-(1,1-difluoroethyl)-6-ethylpyrimidin-4-yl)-1H-pyrazolo[4,3-c]pyridine (Preparation 646, 300 mg, 0.745 mmol) in DMSO (5 mL) was added N,N-dimethylpyrrolidine-3-amine (209.1 mg, 1.12 mmol), CuI (14.2 mg, 74.5 μmol), K₃PO₄ (474.5 mg, 2.24 mmol) and L-proline (8.6 mg, 74.5 μmol) and the reaction was stirred at 100° C. for 2 h under N₂. The mixture was concentrated and purified by prep-HPLC-C (58% to 78% MeCN) to give 1-(6-chloro-1-(2-(1,1-difluoroethyl)-6-ethylpyrimidin-4-yl)-1H-pyrazolo[4,3-c]pyridin-3-yl)-N,N-dimethylpyrrolidin-3-amine (50 mg, crude) as a white solid. LCMS m/z=436.3 [M+H]⁺

### Preparation 701: 1-(6-chloro-1-(2-(1,1-difluoroethyl)-6-methylpyrimidin-4-yl)-1H-pyrazolo[4,3-c]pyridin-3-yl)-N,N,3-trimethylpyrrolidin-3-amine

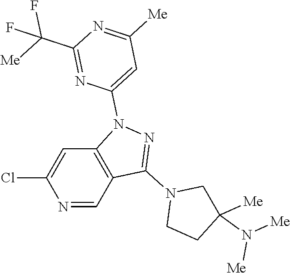 [SMILES: Cc1cc(-n2nc(N3CCC(C)(N(C)C)C3)c3cnc(Cl)cc32)nc(C(C)(F)F)n1]

To a solution of 3-bromo-6-chloro-1-(2-(1,1-difluoroethyl)-6-methylpyrimidin-4-yl)-1H-pyrazolo[4,3-c]pyridine (Preparation 647, 500 mg, 1.29 mmol) in DMSO (10 mL) was added compound N,N,3-trimethylpyrrolidin-3-amine (247.46 mg, 1.93 mmol), CuI (49.0 mg, 0.257 mmol), K₃PO₄ (819.4 mg, 3.86 mmol) and 2,6-DFPAO (51.8 mg, 0.257 mmol) and the reaction mixture was stirred at 100° C. for 16 h under N₂. The mixture was quenched with H₂O (20 mL) and extracted with EtOAc (20 mL×2). The combined organic phase was washed with brine (20 mL×2), dried over Na₂SO₄ and filtered. The filtrate was concentrated under reduced pressure and the residue was purified by chromatography on silica gel (PE/EtOAc=1/1) to give 1-(6-chloro-1-(2-(1,1-difluoroethyl)-6-methylpyrimidin-4-yl)-1H-pyrazolo[4,3-c]pyridin-3-yl)-N,N,3-trimethylpyrrolidin-3-amine (500 mg, 89.2% yield) as a yellow solid. LCMS m/z=436.3 [M+H]⁺

### Preparation 702: 1-(6-chloro-1-(6-(1,1-difluoroethyl)pyrazin-2-yl)-1H-pyrazolo[4,3-c]pyridin-3-yl)-N,N,3-trimethylpyrrolidin-3-amine

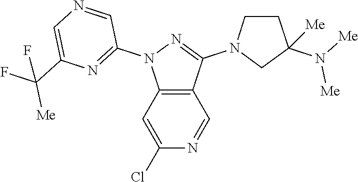 [SMILES: CN(C)C1(C)CCN(c2nn(-c3cncc(C(C)(F)F)n3)c3cc(Cl)ncc23)C1]

1-(6-Chloro-1-(6-(1,1-difluoroethyl)pyrazin-2-yl)-1H-pyrazolo[4,3-c]pyridin-3-yl)-N,N,3-trimethylpyrrolidin-3-amine was obtained as a yellow solid, 220 mg, 97.7% from 3-bromo-6-chloro-1-(6-(1,1-difluoroethyl)pyrazin-2-yl)-1H-pyrazolo[4,3-c]pyridine (Preparation 648) and N,N,3-trimethylpyrrolidin-3-amine following the procedure described in Preparation 701. ¹H NMR: (400 MHz, CDCl₃) δ: ppm 9.30 (s, 1H), 8.85 (s, 1H), 8.67 (s, 1H), 8.44 (s, 1H), 3.69-4.00 (m, 4H), 2.43 (d, J=5.96 Hz, 5H), 2.06-2.18 (m, 5H), 1.25 (s, 3H).

### Preparation 703: 1-(6-chloro-1-(2-(1,1-difluoroethyl)-6-ethylpyrimidin-4-yl)-1H-pyrazolo[4,3-c]pyridin-3-yl)-N,N,3-trimethylpyrrolidin-3-amine

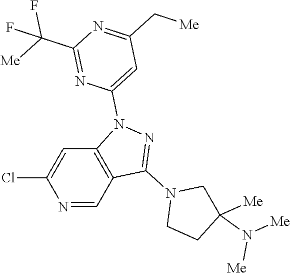 [SMILES: CCc1cc(-n2nc(N3CCC(C)(N(C)C)C3)c3cnc(Cl)cc32)nc(C(C)(F)F)n1]

1-(6-Chloro-1-(2-(1,1-difluoroethyl)-6-ethylpyrimidin-4-yl)-1H-pyrazolo[4,3-c]pyridin-3-yl)-N,N,3-trimethylpyrrolidin-3-amine was obtained as a white solid, 118.2 mg, 70.5%, from 3-bromo-6-chloro-1-(2-(1,1-difluoroethyl)-6-ethylpyrimidin-4-yl)-1H-pyrazolo[4,3-c]pyridine (Preparation 646) and N,N,3-trimethylpyrrolidin-3-amine following the procedure described in Preparation 701. ¹H NMR: (500 MHz, CDCl₃) δ ppm: 8.83 (s, 1H), 8.70 (s, 1H), 7.63 (s, 1H), 3.94-3.97 (m, 1H), 3.83-3.85 (m, 1H), 3.68-3.69 (m, 2H), 2.89-2.92 (m, 2H), 2.38 (s, 6H), 2.21-2.23 (m, 1H), 2.13 (t, J=18.5 Hz, 3H), 2.06-2.08 (m, 1H), 1.38 (t, J=7.5 Hz, 3H), 1.18 (s, 3H).

### Preparation 704: tert-butyl (1-(6-chloro-1-(2-(1,1-difluoroethyl)pyrimidin-4-yl)-1H-pyrazolo[4,3-c]pyridin-3-yl)-5-methylpyrrolidin-3-yl)(methyl)carbamate

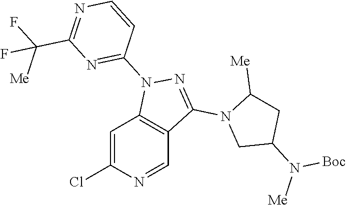 [SMILES: CC1CC(N(C)C(=O)OC(C)(C)C)CN1c1nn(-c2ccnc(C(C)(F)F)n2)c2cc(Cl)ncc12]

To a solution of tert-butyl (1-(6-chloro-1-(2-(1,1-difluoroethyl)pyrimidin-4-yl)-1H-pyrazolo[4,3-c]pyridin-3-yl)-5-methylpyrrolidin-3-yl)carbamate (Preparation 659, 55 mg, 0.111 mmol) in THF (2 mL) was added NaH (8.9 mg, 0.223 mmol, 60% purity) and the mixture was stirred for 30 min. MeI (31.6 mg, 0.223 mmol) was added and the reaction was stirred at 25° C. for 16 h. The mixture was quenched with H₂O (10 mL) and extracted with EtOAc (10 mL×2). The combined organic phase was washed with brine (10 mL×2), dried over Na₂SO₄ and filtered. The reaction was concentrated to give tert-butyl (1-(6-chloro-1-(2-(1,1-difluoroethyl)pyrimidin-4-yl)-1H-pyrazolo[4,3-c]pyridin-3-yl)-5-methylpyrrolidin-3-yl)(methyl)carbamate (30 mg, 53.0% yield) as a yellow solid. LCMS m/z=508.1 [M+H]⁺

### Preparation 705: 6-chloro-3-(3,3-difluorocyclobutyl)-1-(6-(1,1-difluoroethyl)pyridin-2-yl)-1H-pyrazolo[4,3-c]pyridine

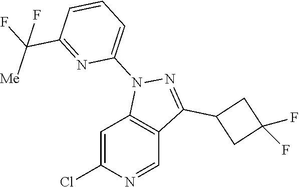 [SMILES: CC(F)(F)c1cccc(-n2nc(C3CC(F)(F)C3)c3cnc(Cl)cc32)n1]

A mixture of 2-bromo-6-(1,1-difluoroethyl)pyridine (62.3 mg, 0.28 mmol), 6-chloro-3-(3,3-difluorocyclobutyl)-1H-pyrazolo[4,3-c]pyridine (Preparation 645, 57 mg, 0.234 mmol), CuI (8.91 mg, 46.8 μmol) and N,N′-dimethylethane-1,2-diamine (8.25 mg, 93.6 μmol) in dioxane (2.69 mL) was sparged with N₂ for 10 min. K₂CO₃ (64.7 mg, 0.468 mmol) was added and the reaction was sparged for 10 min with N₂. The reaction was heated at 100° C. overnight. The cooled reaction mixture was purified by silica gel chromatography eluting with EtOAc:EtOH 3:1 to give 6-chloro-3-(3,3-difluorocyclobutyl)-1-(6-(1,1-difluoroethyl)pyridin-2-yl)-1H-pyrazolo[4,3-c]pyridine, 74 mg of white solid. LCMS m/z=385 [M+H]⁺

### Preparation 706: methyl 6-chloro-1-(6-(1,1-difluoroethyl)pyridin-2-yl)-1H-pyrazolo[4,3-c]pyridine-3-carboxylate

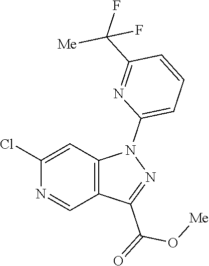 [SMILES: COC(=O)c1nn(-c2cccc(C(C)(F)F)n2)c2cc(Cl)ncc12]

A mixture of methyl 6-chloro-1H-pyrazolo[4,3-c]pyridine-3-carboxylate (200 mg, 0.945 mmol), 2-bromo-6-(1,1-difluoroethyl)pyridine (315 mg, 1.42 mmol) and CuI (18 mg, 0.095 mmol) in dioxane (8 mL) was purged with N₂ and the reaction was heated at 100° C. for 1 h. The cooled mixture was diluted with EtOAc, washed with water and brine. The organic layer was separated, dried and concentrated. The crude was purified by chromatography on silica gel (0-80% EtOAc in heptane) to give methyl 6-chloro-1-(6-(1,1-difluoroethyl)pyridin-2-yl)-1H-pyrazolo[4,3-c]pyridine-3-carboxylate (196 mg, 58% yield) as a white powder. LCMS m/z=353 [M+H]⁺

### Preparation 707: (6-chloro-1-(6-(1,1-difluoroethyl)pyridin-2-yl)-1H-pyrazolo[4,3-c]pyridin-3-yl)methanol

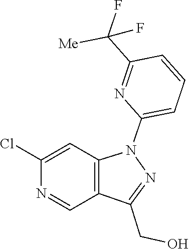

To a mixture of methyl 6-chloro-1-(6-(1,1-difluoroethyl)pyridin-2-yl)-1H-pyrazolo[4,3-c]pyridine-3-carboxylate (Preparation 706, 400 mg, 1.13 mmol) in MeOH (4 mL) and THF (2 mL) was added NaBH₄ (215 mg, 5.67 mmol) at 0° C. The reaction was stirred at rt for 3 h. The reaction mixture was concentrated in vacuo and the crude was purified by chromatography on silica gel (0-50% EtOAc-EtOH 3:1 with 2% NH₄OH in heptane) to give (6-chloro-1-(6-(1,1-difluoroethyl)pyridin-2-yl)-1H-pyrazolo[4,3-c]pyridin-3-yl)methanol (282 mg, 76% yield) as a white solid. LCMS m/z=325 [M+H]⁺

### Preparation 708: 6-chloro-1-(6-(1,1-difluoroethyl)pyridin-2-yl)-3-(methoxymethyl)-1H-pyrazolo[4,3-c]pyridine

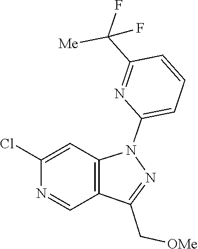

To a mixture of (6-chloro-1-(6-(1,1-difluoroethyl)pyridin-2-yl)-1H-pyrazolo[4,3-c]pyridin-3-yl)methanol (Preparation 707, 100 mg, 0.308 mmol) in DMF (2 mL) was added NaH (15 mg, 0.616 mmol, 60% in oil), followed by MeI (219 mg, 1.54 mmol) and the reaction was stirred at rt overnight. The reaction was quenched with MeOH and the crude mixture was purified by chromatography on silica gel (0-80% EtOAc in heptane) to give 6-chloro-1-(6-(1,1-difluoroethyl)pyridin-2-yl)-3-(methoxymethyl)-1H-pyrazolo[4,3-c]pyridine (52 mg, 44% yield) as white powder. LCMS m/z=339 [M+H]⁺

### Preparation 709: 6-chloro-1-(6-(1,1-difluoroethyl)pyridin-2-yl)-1H-pyrazolo[4,3-c]pyridine-3-carboxylic acid

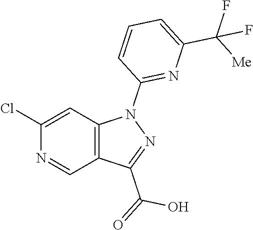 [SMILES: CC(F)(F)c1cccc(-n2nc(C(=O)O)c3cnc(Cl)cc32)n1]

To a solution of methyl 6-chloro-1-(6-(1,1-difluoroethyl)pyridin-2-yl)-1H-pyrazolo[4,3-c]pyridine-3-carboxylate (Preparation 706, 190 mg, 0.539 mmol) in MeOH (2 mL) and THE (2 mL) was added NaOH (3 M, 377 μL) and the reaction stirred at rt overnight. HCl (2 M, 565 μL) was added, the mixture stirred at rt for 5 min and then evaporated under reduced pressure to give 6-chloro-1-(6-(1,1-difluoroethyl)pyridin-2-yl)-1H-pyrazolo[4,3-c]pyridine-3-carboxylic acid, crude. LCMS m/z=339 [M+H]⁺

### Preparation 710: 1-(6-chloro-1-(6-(1,1-difluoroethyl)pyridin-2-yl)-1H-pyrazolo[4,3-c]pyridin-3-yl)ethan-1-one

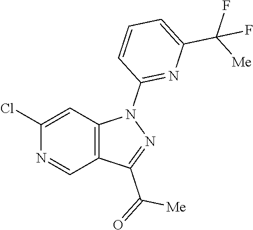 [SMILES: CC(=O)c1nn(-c2cccc(C(C)(F)F)n2)c2cc(Cl)ncc12]

Step 1: To a mixture of 6-chloro-1-(6-(1,1-difluoroethyl)pyridin-2-yl)-1H-pyrazolo[4,3-c]pyridine-3-carboxylic acid (Preparation 709, 180 mg, 0.531 mmol), N-methoxymethanamine hydrochloride (62 mg, 0.638 mmol) and HATU (263 mg, 0.691 mmol) in DMF (3 mL) was added DIPEA (555 μL, 3.19 mmol) and the reaction mixture stirred at rt overnight. The mixture was diluted with EtOAc, washed with water (3×), then brine. The organic layer was separated, dried and concentrated. The crude was purified by chromatography on silica gel (0-80% EtOAc in heptane) to give 6-chloro-1-(6-(1,1-difluoroethyl)pyridin-2-yl)-N-methoxy-N-methyl-1H-pyrazolo[4,3-c]pyridine-3-carboxamide (172 mg, 84% yield) as a white solid.

Step 2: A solution of 6-chloro-1-(6-(1,1-difluoroethyl)pyridin-2-yl)-N-methoxy-N-methyl-1H-pyrazolo[4,3-c]pyridine-3-carboxamide (170 mg, 0.445 mmol) in THF (4 mL) was cooled to −78° C. Chloro(methyl)magnesium (3 M, 222.7 μL) was added and the reaction slowly warmed from −78° C. to rt overnight. The reaction was quenched with MeOH and the mixture was purified by chromatography on silica gel (0-80% EtOAc in heptane) to give 1-(6-chloro-1-(6-(1,1-difluoroethyl)pyridin-2-yl)-1H-pyrazolo[4,3-c]pyridin-3-yl)ethan-1-one (115 mg, 76% yield) as a white solid. LCMS m/z=337 [M+H]⁺

### Preparation 711: 1-(6-chloro-1-(6-(1,1-difluoroethyl)pyridin-2-yl)-1H-pyrazolo[4,3-c]pyridin-3-yl)ethan-1-ol

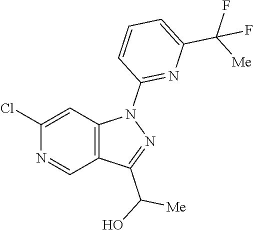 [SMILES: CC(O)c1nn(-c2cccc(C(C)(F)F)n2)c2cc(Cl)ncc12]

To a solution of 1-(6-chloro-1-(6-(1,1-difluoroethyl)pyridin-2-yl)-1H-pyrazolo[4,3-c]pyridin-3-yl)ethan-1-one (Preparation 710, 110 mg, 0.327 mmol) in MeOH (4 mL) was added NaBH₄ (15 mg, 0.392 mmol) and the reaction stirred at rt for 2 h. The mixture was concentrated in vacuo, the crude was diluted with EtOAc, washed with water, then brine. The organic layer was dried and evaporated under reduced pressure to give 1-(6-chloro-1-(6-(1,1-difluoroethyl)pyridin-2-yl)-1H-pyrazolo[4,3-c]pyridin-3-yl)ethan-1-ol (108 mg, 97% yield) as a white solid. LCMS m/z=339 [M+H]⁺

### Preparation 712: 6-chloro-1-(6-(1,1-difluoroethyl)pyridin-2-yl)-3-(1-methoxyethyl)-1H-pyrazolo[4,3-c]pyridine

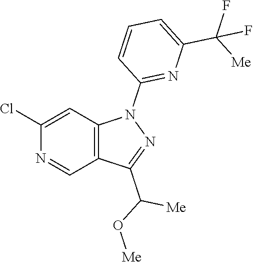 [SMILES: COC(C)c1nn(-c2cccc(C(C)(F)F)n2)c2cc(Cl)ncc12]

A solution of 1-(6-chloro-1-(6-(1,1-difluoroethyl)pyridin-2-yl)-1H-pyrazolo[4,3-c]pyridin-3-yl)ethan-1-ol (Preparation 711, 100 mg, 0.295 mmol) in THF (3 mL) was cooled to 0° C. NaH (18 mg, 0.443 mmol, 60% in mineral oil) was added and the mixture was stirred at 0° C. for 30 min. Iodomethane (92 μL, 1.48 mmol) was added and the reaction was stirred at rt overnight. The reaction was quenched with brine and extracted with EtOAc. The organic layer was then separated, dried and concentrated. The crude was purified by chromatography on silica gel (0-100% EtOAc in heptane) to give 6-chloro-1-(6-(1,1-difluoroethyl)pyridin-2-yl)-3-(1-methoxyethyl)-1H-pyrazolo[4,3-c]pyridine (29 mg, 27% yield). LCMS m/z=353 [M+H]⁺

### Preparation 713: 6-chloro-1-(6-(1,1-difluoroethyl)pyridin-2-yl)-7-fluoro-1H-pyrazolo[4,3-c]pyridine

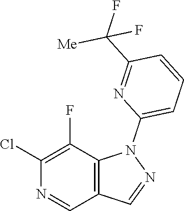 [SMILES: CC(F)(F)c1cccc(-n2ncc3cnc(Cl)c(F)c32)n1]

6-Chloro-1-(6-(1,1-difluoroethyl)pyridin-2-yl)-7-fluoro-1H-pyrazolo[4,3-c]pyridine was obtained as a white powder, 82 mg, 64% from 6-chloro-7-fluoro-1H-pyrazolo[4,3-c]pyridine and 2-bromo-6-(1,1-difluoroethyl)pyridine, following a similar procedure to that described in Preparation 706. LCMS m/z=312.9 [M+H]⁺

### Preparation 714: tert-butyl 3-(6-(6-chloro-3-methyl-1H-pyrazolo[4,3-c]pyridin-1-yl)pyridin-2-yl)-3-fluoroazetidine-1-carboxylate

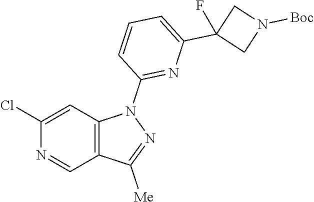 [SMILES: Cc1nn(-c2cccc(C3(F)CN(C(=O)OC(C)(C)C)C3)n2)c2cc(Cl)ncc12]

A mixture of 6-chloro-3-methyl-1H-pyrazolo[4,3-c]pyridine (250 mg, 1.49 mmol), tert-butyl 3-fluoro-3-(6-fluoropyridin-2-yl)azetidine-1-carboxylate (Preparation 439, 403 mg, 1.49 mmol) and K₂CO₃ (412 mg, 2.98 mmol) in DMSO (4 mL) was heated in open air at 90° C. overnight. The cooled mixture was diluted with EtOAc, washed with water (3×), then brine.

The organic layer was separated, dried and concentrated. The crude was purified by chromatography on silica gel (0-80% EtOAc in heptane) to give tert-butyl 3-(6-(6-chloro-3-methyl-1H-pyrazolo[4,3-c]pyridin-1-yl)pyridin-2-yl)-3-fluoroazetidine-1-carboxylate (408 mg, 65% yield) as a white powder. LCMS m/z=418 [M+H]⁺

### Preparation 715: 6-chloro-1-(6-(3-fluoroazetidin-3-yl)pyridin-2-yl)-3-methyl-1H-pyrazolo[4,3-c]pyridine

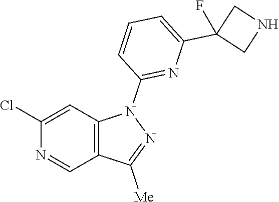 [SMILES: Cc1nn(-c2cccc(C3(F)CNC3)n2)c2cc(Cl)ncc12]

To a solution of tert-butyl 3-(6-(6-chloro-3-methyl-1H-pyrazolo[4,3-c]pyridin-1-yl)pyridin-2-yl)-3-fluoroazetidine-1-carboxylate (Preparation 714, 190 mg, 0.455 mmol) in DCM (3 mL) was added TFA (0.6 mL) and the reaction was stirred at rt overnight. The reaction mixture was concentrated under vacuum. The residue was diluted with EtOAc, washed with aq. NaHCO₃. The organic layer was separated, dried and concentrated to give 6-chloro-1-(6-(3-fluoroazetidin-3-yl)pyridin-2-yl)-3-methyl-1H-pyrazolo[4,3-c]pyridine, as a white powder. LCMS m/z=318 [M+H]⁺

### Preparation 716: 1-(3-(6-(6-chloro-3-methyl-1H-pyrazolo[4,3-c]pyridin-1-yl)pyridin-2-yl)-3-fluoroazetidin-1-yl)ethan-1-one

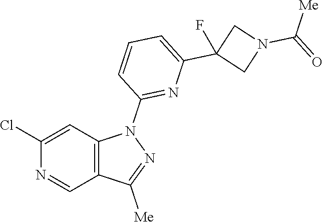 [SMILES: CC(=O)N1CC(F)(c2cccc(-n3nc(C)c4cnc(Cl)cc43)n2)C1]

To a solution of 6-chloro-1-(6-(3-fluoroazetidin-3-yl)pyridin-2-yl)-3-methyl-1H-pyrazolo[4,3-c]pyridine (Preparation 715, 50 mg, 91.6 μmol) in DCM (1 mL) was added DIPEA (96 μL, 0.55 mmol), followed by acetyl chloride (72 mg, 0.916 mmol) and the reaction stirred at rt for 30 min. The reaction was diluted with EtOAc, washed with water, then brine. The organic layer was separated, dried and concentrated to give 1-(3-(6-(6-chloro-3-methyl-1H-pyrazolo[4,3-c]pyridin-1-yl)pyridin-2-yl)-3-fluoroazetidin-1-yl)ethan-1-one (31 mg, 75% yield) as a yellowish solid. LCMS m/z=360 [M+H]⁺

### Preparation 717: 6-chloro-1-(6-(3-fluoro-1-(methylsulfonyl)azetidin-3-yl)pyridin-2-yl)-3-methyl-1H-pyrazolo[4,3-c]pyridine

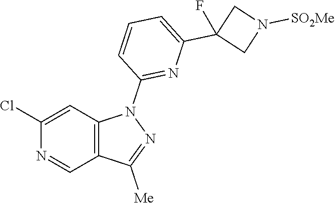 [SMILES: Cc1nn(-c2cccc(C3(F)CN(S(C)(=O)=O)C3)n2)c2cc(Cl)ncc12]

6-Chloro-1-(6-(3-fluoro-1-(methylsulfonyl)azetidin-3-yl)pyridin-2-yl)-3-methyl-1H-pyrazolo[4,3-c]pyridine was obtained as a solid, 41 mg, 90%, from 6-chloro-1-(6-(3-fluoroazetidin-3-yl)pyridin-2-yl)-3-methyl-1H-pyrazolo[4,3-c]pyridine (Preparation 715) and methanesulfonyl chloride, following the procedure described in Preparation 716. LCMS m/z=396 [M+H]⁺

### Preparations 718 and 719: 5-bromo-1-(difluoromethyl)-3-methylpyridin-2(1H)-one and 5-bromo-2-(difluoromethoxy)-3-methyl-1,2-dihydropyridine

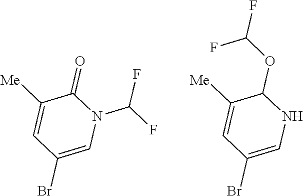 [SMILES: Cc1cc(Br)cn(C(F)F)c1=O ; CC1=CC(Br)=CNC1OC(F)F]

To a solution of 5-bromo-3-methylpyridin-2(1H)-one (1.0 g, 5.32 mmol) and LiBr (923.8 mg, 10.64 mmol) in DMSO (10 mL) was added NaH (234.0 mg, 5.85 mmol, 60% purity) and sodium 2-chloro-2,2-difluoroacetate (1.6 g, 10.64 mmol) at 0° C. The reaction mixture was stirred at 80° C. for 16 h under N₂. The reaction was quenched with H₂O (20 mL) and extracted with EtOAc (10 mL×3). The organic phase was washed with brine (20 mL), dried with Na₂SO₄, filtered and concentrated. The residue was purified by column chromatography (PE/EtOAc=15/1 to 3/1) on silica gel to give 5-bromo-1-(difluoromethyl)-3-methylpyridin-2(1H)-one (300 mg, 23.7% yield) as colorless oil. ¹H NMR: (400 MHz, CDCl₃) δ ppm: 7.65 (t, J=60.4 Hz, 1H), 7.45 (d, J=2.0 Hz, 1H), 7.42 (d, J=2.4 Hz, 1H), 2.14 (s, 3H).

and 5-bromo-2-(difluoromethoxy)-3-methyl-1,2-dihydropyridine (330.0 mg, 26.1% yield) as colorless oil. ¹H NMR: (400 MHz, CDCl₃) δ ppm: 8.06 (d, J=2.4 Hz, 1H), 7.66 (d, J=1.6 Hz, 1H), 7.42 (t, J=73.2 Hz, 1H), 2.26 (s, 3H).

### Preparation 720: 6-chloro-1-(4-(1,1-difluoroethyl)pyrimidin-2-yl)-3-iodo-1H-pyrrolo[3,2-c]pyridine

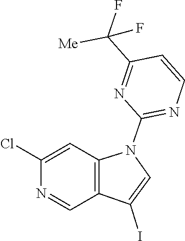

A mixture of 6-chloro-3-iodo-1H-pyrrolo[3,2-c]pyridine (500 mg, 1.80 mmol), 2-chloro-4-(1,1-difluoroethyl)pyrimidine (353 mg, 1.98 mmol), Cs₂CO₃ (1.17 g, 3.59 mmol) and DMSO (8 mL) was heated at 80° C. for 2 h. The reaction was diluted with EtOAc, washed with water (3×), then brine. The organic layer was separated, dried and concentrated. The crude was triturated with MeOH to give 6-chloro-1-(4-(1,1-difluoroethyl)pyrimidin-2-yl)-3-iodo-1H-pyrrolo[3,2-c]pyridine (388 mg, 51% yield). LCMS m/z=421 [M+H]⁺

### Preparation 721: 6-chloro-1-(2-(1,1-difluoroethyl)-6-ethylpyrimidin-4-yl)-1H-pyrrolo[3,2-c]pyridine-3-carbaldehyde

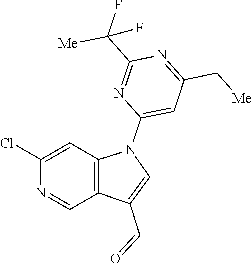

To a solution of 6-chloro-1H-pyrrolo[3,2-c]pyridine-3-carbaldehyde (750 mg, 4.15 mmol) and 4-chloro-2-(1,1-difluoroethyl)-6-ethylpyrimidine (Preparation 390, 858.1 mg, 4.15 mmol) in DMF (15 mL) was added K₂CO₃ (1.15 g, 8.31 mmol) and the reaction mixture was stirred at 80° C. for 2 h. The cooled mixture was diluted with water, the resulting solid filtered off, washed with water and vacuum dried to give 6-chloro-1-(2-(1,1-difluoroethyl)-6-ethylpyrimidin-4-yl)-1H-pyrrolo[3,2-c]pyridine-3-carbaldehyde (1.18 g, 80.8% yield) as a solid. LCMS m/z=351.1 [M+H]⁺

### Preparation 722: 6-chloro-1-(6-(1,1-difluoroethyl)pyridin-2-yl)-1H-pyrrolo[3,2-c]pyridine-3-carbaldehyde

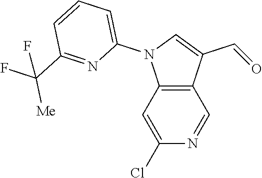 [SMILES: CC(F)(F)c1cccc(-n2cc(C=O)c3cnc(Cl)cc32)n1]

A mixture of 2-bromo-6-(1,1-difluoroethyl)pyridine (295.07 mg, 1.33 mmol), 6-chloro-1H-pyrrolo[3,2-c]pyridine-3-carbaldehyde (200 mg, 1.11 mmol), dioxane (9.72 mL) and K₂CO₃ (306.13 mg, 2.21 mmol) in CuI (26 mg, 0.137 mmol) was sparged with N₂ for 10 min and the reaction was heated at 70° C. for 20 h. The cooled mixture was filtered and the residue purified by silica gel column chromatography (Heptane to EtOAc:EtOH 3:1) to give 6-chloro-1-(6-(1,1-difluoroethyl)pyridin-2-yl)-1H-pyrrolo[3,2-c]pyridine-3-carbaldehyde as a white solid, 176 mg. LCMS m/z=322 [M+H]⁺

### Preparation 723: 6-chloro-1-(6-(1,1-difluoroethyl)pyridin-2-yl)-3-(difluoromethyl)-1H-pyrrolo[3,2-c]pyridine

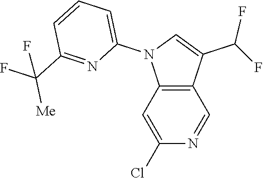 [SMILES: CC(F)(F)c1cccc(-n2cc(C(F)F)c3cnc(Cl)cc32)n1]

DAST (501.0 mg, 3.11 mmol) and N,N-diethylethanamine trihydrofluoride (50.1 mg, 0.311 mmol) were added to a solution of 6-chloro-1-(6-(1,1-difluoroethyl)pyridin-2-yl)-1H-pyrrolo[3,2-c]pyridine-3-carbaldehyde (Preparation 722, 100 mg, 0.311 mmol) in DCM (5 mL) cooled by an ice bath. The reaction was warmed to rt and stirred overnight. The solvent was removed and the residue purified by silica gel chromatography, eluting with heptane to 3:1 EtOAc:EtOH to give 6-chloro-1-(6-(1,1-difluoroethyl)pyridin-2-yl)-3-(difluoromethyl)-1H-pyrrolo[3,2-c]pyridine, 48 mg. LCMS m/z=344 [M+H]⁺

### Preparation 724: N-(1-(2-(1,1-difluoroethyl)-6-ethylpyrimidin-4-yl)-3-formyl-1H-pyrrolo[3,2-c]pyridin-6-yl)acetamide

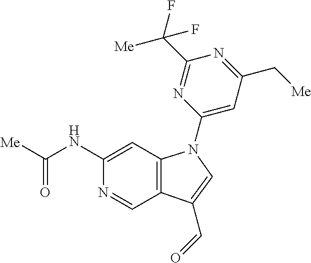 [SMILES: CCc1cc(-n2cc(C=O)c3cnc(NC(C)=O)cc32)nc(C(C)(F)F)n1]

A mixture of 6-chloro-1-(2-(1,1-difluoroethyl)-6-ethylpyrimidin-4-yl)-1H-pyrrolo[3,2-c]pyridine-3-carbaldehyde (Preparation 721, 1.18 g, 3.35 mmol), acetamide (989.4 mg, 16.75 mmol) and Cs₂CO₃ (2.18 g, 6.70 mmol) in dry dioxane (15 mL) was sparged with N2 for 5 min. BrettPhos Pd G3 (151.84 mg, 0.168 mmol) was added and the resulting mixture was stirred at 90° C. for 2 h. The reaction mixture was cooled to rt, diluted with EtOAc and water. A solid formed, which was filtered and washed with EtOAc. The filtrate was separated, the organic layer was washed with brine, the organic layer was back-extracted with EtOAc, the combined organic extracts were dried (MgSO₄), filtered and concentrated in vacuo. The crude product was purified by chromatography on silica gel (0-10% MeOH in DCM) to give N-(1-(2-(1,1-difluoroethyl)-6-ethylpyrimidin-4-yl)-3-formyl-1H-pyrrolo[3,2-c]pyridin-6-yl)acetamide (580 mg, 46.4% yield) as a brown solid. LCMS m/z=378.2 [M+H]⁺

### Preparation 725: 6-chloro-1-(4-(1,1-difluoroethyl)pyrimidin-2-yl)-3-(4-methylpiperazin-1-yl)-1H-pyrrolo[3,2-c]pyridine

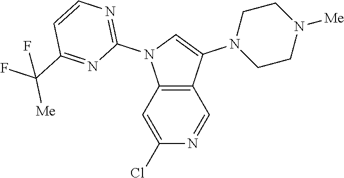 [SMILES: CN1CCN(c2cn(-c3nccc(C(C)(F)F)n3)c3cc(Cl)ncc23)CC1]

To a solution of 1-methylpiperazine (179 mg, 1.78 mmol) in DMSO (3.0 mL) was added anhydrous K₂CO₃ (246 mg, 1.78 mmol), DL-Proline (25 mg, 0.214 mmol), CuI (20 mg, 0.107 mmol) and the mixture stirred at rt for 5 min. 6-Chloro-1-(4-(1,1-difluoroethyl)pyrimidin-2-yl)-3-iodo-1H-pyrrolo[3,2-c]pyridine (Preparation 720, 150 mg, 0.357 mmol) was added, the reaction mixture was purged with N₂, then heated at 80° C. for 6 h. The cooled mixture was filtered and the filtrate purified by HPLC to give 6-chloro-1-(4-(1,1-difluoroethyl)pyrimidin-2-yl)-3-(4-methylpiperazin-1-yl)-1H-pyrrolo[3,2-c]pyridine (92 mg, 50% yield) as a white solid. LCMS m/z=393 [M+H]⁺

### Preparation 726: N-(1-(6-chloro-2-(1,1-difluoroethyl)pyrimidin-4-yl)-3-ethyl-1H-pyrrolo[3,2-c]pyridin-6-yl)acetamide

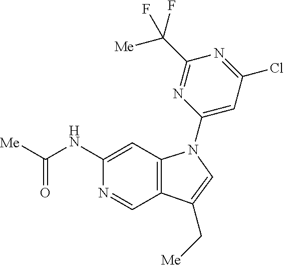 [SMILES: CCc1cn(-c2cc(Cl)nc(C(C)(F)F)n2)c2cc(NC(C)=O)ncc12]

To a vial was added N-(3-ethyl-1H-pyrrolo[3,2-c]pyridin-6-yl)acetamide (1 g, 4.92 mmol), 4,6-dichloro-2-(1,1-difluoroethyl)pyrimidine (1.26 g, 5.90 mmol), Cs₂CO₃ (3.21 g, 9.84 mmol) and DMF (22.9 mL) and the reaction was stirred at rt overnight. The reaction was diluted with water (10:1 v:v) and the resulting solid filtered off and dried to give N-(1-(6-chloro-2-(1,1-difluoroethyl)pyrimidin-4-yl)-3-ethyl-1H-pyrrolo[3,2-c]pyridin-6-yl)acetamide, 1.16 g as a tan solid. LCMS m/z=380 [M+H]⁺

### Preparation 727: N-(1-(6-chloro-2-(1,1-difluoroethyl)pyrimidin-4-yl)-1H-pyrrolo[3,2-c]pyridin-6-yl)acetamide

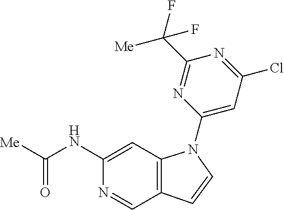 [SMILES: CC(=O)Nc1cc2c(ccn2-c2cc(Cl)nc(C(C)(F)F)n2)cn1]

N-(1-(6-Chloro-2-(1,1-difluoroethyl)pyrimidin-4-yl)-1H-pyrrolo[3,2-c]pyridin-6-yl)acetamide was obtained, from 4,6-dichloro-2-(1,1-difluoroethyl)pyrimidine and N-(1H-pyrrolo[3,2-c]pyridin-6-yl)acetamide, following a similar procedure to that described in Preparation 726.

### Preparation 728: tert-butyl 6-chloro-1H-imidazo[4,5-c]pyridine-1-carboxylate

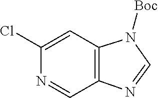 [SMILES: CC(C)(C)OC(=O)n1cnc2cnc(Cl)cc21]

To a mixture of 6-chloro-1H-imidazo[4,5-c]pyridine (500 mg, 3.26 mmol) in DCM (20 mL) was added tert-butoxycarbonyl tert-butyl carbonate (1.42 g, 6.52 mmol), followed by DMAP (40 mg, 0.326 mmol) and the reaction was stirred at rt overnight. The reaction mixture was concentrated and the crude was purified by chromatography on silica gel (0-80% EtOAc in heptane) to give tert-butyl 6-chloroimidazo[4,5-c]pyridine-1-carboxylate (402 mg, 48% yield) as a white solid. LCMS m/z=254 [M+H]⁺

### Preparation 729: tert-butyl 6-acetamido-1H-imidazo[4,5-c]pyridine-1-carboxylate

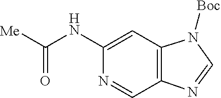 [SMILES: CC(=O)Nc1cc2c(cn1)ncn2C(=O)OC(C)(C)C]

A mixture of tert-butyl 6-chloroimidazo[4,5-c]pyridine-1-carboxylate (Preparation 728, 390 mg, 1.54 mmol), acetamide (182 mg, 3.07 mmol), Cs₂CO₃ (1.00 g, 3.07 mmol) and BrettPhos Pd G4 (70 mg, 0.077 mmol) in dioxane (10 mL) was degassed with N₂, then heated at 100° C. in a sealed tube for 1 h. The cooled mixture was filtered and the filtrate concentrated in vacuo. The crude was purified by chromatography on silica gel (0-70% EtOAc-EtOH 3:1 with 2% NH₄OH in heptane) to give tert-butyl 6-acetamidoimidazo[4,5-c]pyridine-1-carboxylate (332 mg, 78.% yield) as a white powder. LCMS m/z=299 [M+Na]⁺

### Preparation 730: N-(1H-imidazo[4,5-c]pyridin-6-yl)acetamide

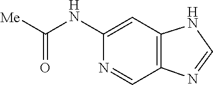 [SMILES: CC(=O)Nc1cc2[nH]cnc2cn1]

A solution of tert-butyl 6-acetamidoimidazo[4,5-c]pyridine-1-carboxylate (Preparation 729, 320 mg, 1.16 mmol) in EtOH (12 mL) was heated at 150° C. in a microwave reactor for 30 min. The cooled mixture was diluted with EtOAc, the mixture was filtered and washed with MeOH. The filtrate was concentrated, and the solid formed was washed with DCM/Heptane (20/80) to give N-(1H-imidazo[4,5-c]pyridin-6-yl)acetamide (167 mg, 81% yield) as an off-white powder. LCMS m/z=177 [M+H]⁺

### Example 1: (R)—N-(1-(4-(3-methoxytetrahydrofuran-3-yl)pyridin-2-yl)-1H-pyrazolo[4,3-c]pyridin-6-yl)acetamide and (S)—N-(1-(4-(3-methoxytetrahydrofuran-3-yl)pyridin-2-yl)-1H-pyrazolo[4,3-c]pyridin-6-yl)acetamide

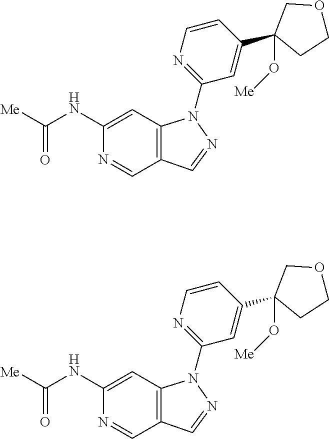 [SMILES: CO[C@@]1(c2ccnc(-n3ncc4cnc(NC(C)=O)cc43)c2)CCOC1 ; CO[C@]1(c2ccnc(-n3ncc4cnc(NC(C)=O)cc43)c2)CCOC1]

To a solution of N-(1H-pyrazolo[4,3-c]pyridin-6-yl)acetamide (Preparation 2, 100 mg, 0.57 mmol) in DMF (5 mL) was added NaH (22.7 mg, 0.57 mmol, 60% purity), then 2-fluoro-4-(3-methoxytetrahydrofuran-3-yl)pyridine (Preparation 48, 111.9 mg, 0.57 mmol) and the reaction stirred at 50° C. for 2 h. The mixture was concentrated to give crude product, which was purified by Prep-HPLC-F (Gradient: 15-45% MeCN) to give Peak 1, (R)—N-(1-(4-(3-methoxytetrahydrofuran-3-yl)pyridin-2-yl)-1H-pyrazolo[4,3-c]pyridin-6-yl)acetamide or (S)—N-(1-(4-(3-methoxytetrahydrofuran-3-yl)pyridin-2-yl)-1H-pyrazolo[4,3-c]pyridin-6-yl)acetamide (10.3 mg, 5.1% yield), as a white solid, LCMS m/z=354.1 [M+H]⁺. ¹H NMR: (400 MHz, CDCl₃) δ ppm: 9.44 (br s, 1H), 8.80 (s, 1H), 8.60 (d, J=5.6 Hz, 1H), 8.24 (d, J=0.8 Hz, 1H), 8.14 (s, 1H), 8.02 (d, J=0.8 Hz, 1H), 7.25-7.24 (m, 1H), 4.21-4.10 (m, 3H), 3.94-3.92 (m, 1H), 3.20 (s, 3H), 2.52-2.50 (m, 1H), 2.39-2.33 (m, 1H), 2.27 (s, 3H).

Peak 2, enantiomer 2, (R)—N-(2-(4-(3-methoxytetrahydrofuran-3-yl)pyridin-2-yl)-2H-pyrazolo[4,3-c]pyridin-6-yl)acetamide or (S)—N-(2-(4-(3-methoxytetrahydrofuran-3-yl)pyridin-2-yl)-2H-pyrazolo[4,3-c]pyridin-6-yl)acetamide (7.0 mg, 3.5% yield).

Peak 3, enantiomer 3, (S)—N-(1-(4-(3-methoxytetrahydrofuran-3-yl)pyridin-2-yl)-1H-pyrazolo[4,3-c]pyridin-6-yl)acetamide or (R)—N-(1-(4-(3-methoxytetrahydrofuran-3-yl)pyridin-2-yl)-1H-pyrazolo[4,3-c]pyridin-6-yl)acetamide (8.2 mg, 4.1% yield). LCMS m/z=354.2 [M+H]⁺. ¹H NMR: (400 MHz, CDCl₃) δ ppm: 8.81 (s, 1H), 8.61-8.63 (m, 1H), 8.25 (d, J=1.2 Hz, 1H), 8.14 (s, 1H), 8.05 (s, 1H), 8.02 (d, J=1.2 Hz, 1H), 7.52-7.56 (m, 1H), 3.74-3.91 (m, 4H), 3.12 (s, 3H), 2.25-2.31 (m, 1H), 2.19 (s, 3H), 2.12-2.15 (m, 1H)

Peak 4, enantiomer 4, (S)—N-(2-(4-(3-methoxytetrahydrofuran-3-yl)pyridin-2-yl)-2H-pyrazolo[4,3-c]pyridin-6-yl)acetamide or (R)—N-(2-(4-(3-methoxytetrahydrofuran-3-yl)pyridin-2-yl)-2H-pyrazolo[4,3-c]pyridin-6-yl)acetamide (3.8 mg, 1.89% yield) as white solid.

### Example 2: N-(1-(6-(2-oxopyrrolidin-1-yl)pyridin-2-yl)-1H-pyrazolo[4,3-c]pyridin-6-yl)acetamide

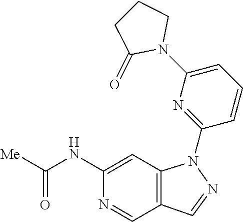 [SMILES: CC(=O)Nc1cc2c(cn1)cnn2-c1cccc(N2CCCC2=O)n1]

Under N₂, a suspension of N-(1H-pyrazolo[4,3-c]pyridin-6-yl)acetamide (Preparation 2, 150 mg, 0.851 mmol), 1-(6-chloropyridin-2-yl)pyrrolidin-2-one (Preparation 88, 167.4 mg, 0.851 mmol), Cs₂CO₃ (554.8 mg, 1.70 mmol), Pd₂(dba)₃ (54.6 mg, 0.060 mmol) and Xantphos (73.9 mg, 0.128 mmol) in dioxane (5 mL) was stirred at 100° C. for 16 h. The cooled reaction suspension was concentrated in vacuo, the residue was diluted with water (50 mL) and extracted with EtOAc (100 mL×2). The combined organic layer was washed with water (50 mL) and brine (50 mL), dried over Na₂SO₄ and filtered. The filtrate was concentrated and the residue was purified by silica gel chromatography (PE/EtOAc=1/1) to afford a crude product. This was purified by prep-HPLC-C (Gradient: 25-56% MeCN) to afford N-(1-(6-(2-oxopyrrolidin-1-yl)pyridin-2-yl)-1H-pyrazolo[4,3-c]pyridin-6-yl)acetamide (4.2 mg, 1.40% yield) as a white solid. LCMS m/z=337.1 [M+H]⁺. 1H NMR (500 MHz, DMSO-d₆) δ ppm 10.72 (s, 1H), 9.32 (s, 1H), 8.96 (s, 1H), 8.55 (s, 1H), 8.25 (d, J=8.5 Hz, 1H), 8.02 (t, J=8.0 Hz, 1H), 7.75-7.71 (m, 1H), 4.36 (t, J=7.5 Hz, 2H), 2.69-2.65 (m, 2H), 2.19-2.11 (m, 5H).

### Example 3: N-(1-(2-(3-methoxytetrahydrofuran-3-yl)pyridin-4-yl)-1H-pyrazolo[4,3-c]pyridin-6-yl)acetamide

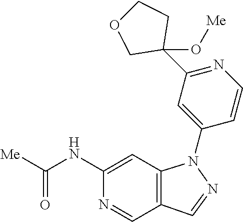 [SMILES: COC1(c2cc(-n3ncc4cnc(NC(C)=O)cc43)ccn2)CCOC1]

N-(1-(2-(3-Methoxytetrahydrofuran-3-yl)pyridin-4-yl)-1H-pyrazolo[4,3-c]pyridin-6-yl)acetamide was obtained as a white solid, 13 mg, 7.9% yield, from N-(1H-pyrazolo[4,3-c]pyridin-6-yl)acetamide (Preparation 2) and 4-chloro-2-(3-methoxytetrahydrofuran-3-yl)pyridine (Preparation 54), following a similar procedure to that described in Example 2. LCMS m/z=354.2 [M+H]⁺. 1H NMR: (500 MHz, CDCl₃) δ ppm: 8.82-8.43 (m, 2H), 8.74 (d, J=5.5 Hz, 1H), 8.31 (s, 1H), 8.15 (s, 1H), 8.04 (d, J=2.0 Hz, 1H), 7.71-7.69 (m, 1H), 4.26-4.12 (m, 4H), 3.36 (s, 3H), 2.73-2.67 (m, 1H), 2.50-2.48 (m, 1H), 2.28 (s, 3H).

### Example 4: N-(1-(6-(3-ethyltetrahydrofuran-3-yl)pyridin-2-yl)-1H-pyrazolo[4,3-c]pyridin-6-yl)acetamide

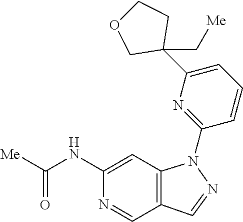 [SMILES: CCC1(c2cccc(-n3ncc4cnc(NC(C)=O)cc43)n2)CCOC1]

N-(1-(6-(3-Ethyltetrahydrofuran-3-yl)pyridin-2-yl)-1H-pyrazolo[4,3-c]pyridin-6-yl)acetamide was obtained as a white solid (17.3 mg, 7%) from N-(1H-pyrazolo[4,3-c]pyridin-6-yl)acetamide (Preparation 2) and 2-chloro-6-(3-ethyltetrahydrofuran-3-yl)pyridine (Preparation 85), following a similar procedure to that described in Example 2. LCMS m/z=352.2 [M+H]⁺. ¹H NMR (500 MHz, MeOH-d₄) δ ppm: 9.41 (s, 1H), 8.87 (s, 1H), 8.37 (s, 1H), 7.93-7.92 (m, 2H), 7.30-7.28 (m, 1H), 4.30-4.28 (m, 1H), 4.20-4.19 (m, 1H), 4.08-4.07 (m, 1H), 3.97-3.96 (m, 1H), 2.67-2.65 (m, 1H), 2.38-2.37 (m, 1H), 2.26 (s, 3H), 2.09-2.07 (m, 2H), 0.82-0.79 (t, J=7.5 Hz, 3H).

### Example 5: N-(1-(4-methoxy-6-(3-methoxytetrahydrofuran-3-yl)pyridin-2-yl)-1H-pyrazolo[4,3-c]pyridin-6-yl)acetamide

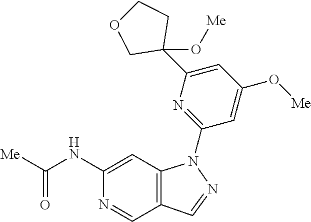 [SMILES: COc1cc(-n2ncc3cnc(NC(C)=O)cc32)nc(C2(OC)CCOC2)c1]

To a solution of 2-chloro-4-methoxy-6-(3-methoxytetrahydrofuran-3-yl)pyridine (Preparation 91, 50 mg, 0.205 mmol) in dioxane (5.0 mL) was added N-(1H-pyrazolo[4,3-c]pyridin-6-yl)acetamide (Preparation 2, 39.8 mg, 0.226 mmol), Pd₂dba₃ (18.8 mg, 0.021 mmol), Xantphos (23.7 mg, 0.041 mmol) and Zn(OAc)₂ (41.4 mg, 0.226 mmol) and the reaction mixture was stirred for 12 h at 120° C. under N₂. The cooled mixture was filtered and the filtrate was concentrated under reduced pressure. The residue was purified by Prep-HPLC-M (Gradient: 20-50%) to give N-(1-(4-methoxy-6-(3-methoxytetrahydrofuran-3-yl)pyridin-2-yl)-1H-pyrazolo[4,3-c]pyridin-6-yl)acetamide (17.62 mg, 19.5% yield) as a yellow solid. LCMS m/z=384.2 [M+H]⁺. 1H NMR (400 MHz, MeOH-d₄) δ ppm 9.26 (s, 1H), 8.83 (s, 1H), 8.63 (s, 1H), 7.65 (d, J=1.6 Hz, 1H), 7.19 (d, J=2.4 Hz, 1H), 4.36-4.32 (m, 1H), 4.23-4.15 (m, 3H), 4.05 (s, 3H), 3.28 (s, 3H), 2.68-2.60 (m, 2H), 2.39 (s, 3H).

### Examples 6 to 17

The compounds in the following table were prepared from N-(1H-pyrazolo[4,3-c]pyridin-6-yl)acetamide (Preparation 2, and the appropriate substituted pyridine following a similar procedure to that described in Example 5.

| Example | |
|---|---|
| No | Name/Structure/Pyridine/Data |
| 6^{A} | N-(1-(6-(3-methoxytetrahydro-3-yl)-4-(oxetan-3-yloxy)pyridin-2-yl)- |
| | 1H-pyrazolo[4,3-c]pyridin-6-yl)acetamide |
| | 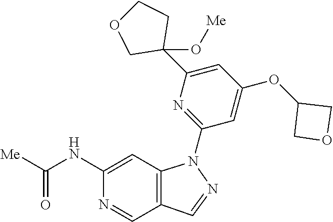 [SMILES: COC1(c2cc(OC3COC3)cc(-n3ncc4cnc(NC(C)=O)cc43)n2)CCOC1] |
| | Pyridine: 2-chloro-6-(3-methoxytetrahydrofuran-3-yl)-4-(oxetan-3-yloxy)pyridine |
| | (Preparation 92) |
| | Prep-HPLC-N (Gradient: 17-57% MeCN) |
| | 18.5 mg, 10.4% yield as a white solid. LCMS m/z = 426.2 [M + H]⁺. 1H |
| | NMR (400 MHz, DMSO-d₆) δ ppm 10.69 (s, 1H), 9.27 (s, 1H), 8.95 (s, 1H), |
| | 8.55 (s, 1H), 7.22 (s, 1H), 6.89 (d, J = 2.0 Hz, 1H), 5.70-5.58 (m, 1H), 4.99 (t, |
| | J = 6.8 Hz, 2H), 4.68-4.60 (m, 2H), 4.24-4.21 (m, 1H), 4.12-4.04 (m, 1H), |
| | 4.03-3.95 (m, 1H), 3.91-3.87 (m, 1H), 3.16 (s, 3H), 2.80-2.70 (m, 1H), 2.57- |
| | 2.52 (m, 1H), 2.15 (s, 3H). |
| 7 | N-(1-(6-(3-methoxytetrahydrofuran-3-yl)-4-(oxetan-2-ylmethoxy)pyridin-2- |
| | yl)-1H-pyrazolo[4,3-c]pyridin-6-yl)acetamide |
| | 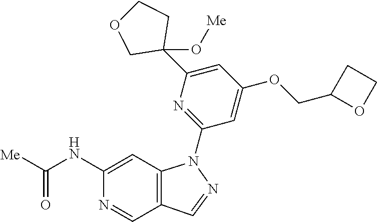 [SMILES: COC1(c2cc(OCC3CCO3)cc(-n3ncc4cnc(NC(C)=O)cc43)n2)CCOC1] |
| | Pyridine: 2-chloro-6-(3-methoxytetrahydrofuran-3-yl)-4-(oxetan-2- |
| | ylmethoxy)pyridine (Preparation 93) |
| | Prep HPLC-C (Gradient: 26-56% MeCN) |
| | 29.9 mg, 7.55% yield as a white solid. LCMS m/z = 440.2 [M + H]⁺ |
| | ¹H NMR: (400 MHz, CDCl₃) δ ppm: 9.35 (s, 1H), 8.77 (s, 1H), 8.21-8.12 |
| | (m, 2H), 7.54-7.53 (m, 1H), 7.09-7.07 (m, 1H), 5.22-5.20 (m, 1H), 4.75-4.67 |
| | (m, 2H), 4.32-4.09 (m, 6H), 3.31 (s, 3H), 3.00-2.55 (m, 4H), 2.25 (s, 3H). |
| 8 | N-(1-(6-(3-methoxytetrahydrofuran-3-yl)-4-(oxetan-3-ylmethoxy)pyridin-2- |
| | yl)-1H-pyrazolo[4,3-c]pyridin-6-yl)acetamide |
| | 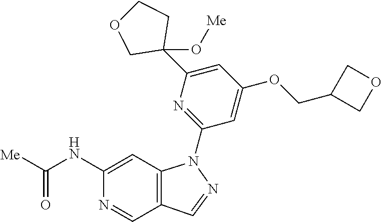 [SMILES: COC1(c2cc(OCC3COC3)cc(-n3ncc4cnc(NC(C)=O)cc43)n2)CCOC1] |
| | Pyridine: 2-chloro-6-(3-methoxytetrahydrofuran-3-yl)-4-(oxetan-3- |
| | ylmethoxy)pyridine (Preparation 94) |
| | Prep-HPLC-C (Gradient: 26-56% MeCN) |
| | 62.3 mg, 19.8% yield as a white solid. LCMS m/z = 440.3 [M + H]⁺ |
| | 1H NMR: (500 MHz, CDCl₃) δ ppm: 9.35 (s, 1H), 8.78 (s, 1H), 8.21 (s, |
| | 1H), 8.07 (s, 1H), 7.50 (d, J = 2.5 Hz, 1H), 7.02 (d, J = 1.5 Hz, 1H), 4.94-4.90 |
| | (m, 2H), 4.62-4.59 (m, 2H), 4.40-4.39 (m, 2H), 4.29-4.27 (m, 3H), 4.25-4.23 |
| | (m, 1H), 3.52-3.49 (m, 1H), 3.32 (s, 3H), 3.00-2.97 (m, 1H), 2.59-2.56 (m, |
| | 1H), 2.26 (s, 3H). |
| 9 | N-(1-(4-((1-methoxypropan-2-yl)oxy)-6-(3-methoxytetrahydrofuran-3- |
| | yl)pyridin-2-yl)-1H-pyrazolo[4,3-c]pyridin-6-yl)acetamide |
| | 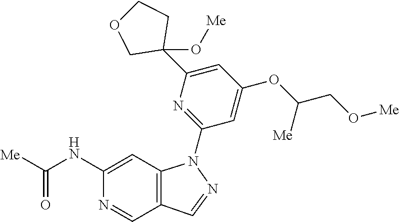 [SMILES: COCC(C)Oc1cc(-n2ncc3cnc(NC(C)=O)cc32)nc(C2(OC)CCOC2)c1] |
| | Pyridine: 2-chloro-4-((1-methoxypropan-2-yl)oxy)-6-(3- |
| | methoxytetrahydrofuran-3-yl)pyridine (Preparation X) |
| | Prep-HPLC-F (Gradient: 30-60% MeCN) |
| | 130.2 mg, 20.8% yield as a white solid. LCMS m/z = 442.3 [M + H]⁺ |
| | 1H NMR: (500 MHz, CDCl₃) δ ppm: 9.34 (s, 1H), 8.76 (s, 1H), 8.31 (br s, |
| | 1H), 8.18 (s, 1H), 7.50 (s, 1H), 7.03-7.02 (m, 1H), 4.82-4.81 (m, 1H), 4.27- |
| | 4.20 (m, 3H), 4.11-4.09 (m, 1H), 3.63-3.62 (m, 1H), 3.58-3.57 (m, 1H), 3.43 |
| | (s, 3H), 3.30 (s, 3H), 2.95-2.94 (m, 1H), 2.56-2.54 (m, 1H), 2.24 (s, 3H), |
| | 1.40 (d, J = 6.0 Hz, 3H). |
| 10 | N-(1-(4-(2-methoxypropoxy)-6-(3-methoxytetrahydrofuran-3-yl)pyridin-2- |
| | yl)-1H-pyrazolo[4,3-c]pyridin-6-yl)acetamide |
| | 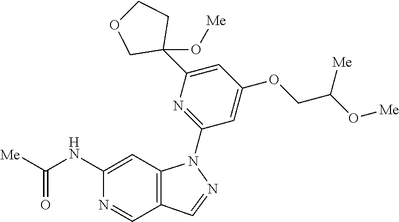 [SMILES: COC(C)COc1cc(-n2ncc3cnc(NC(C)=O)cc32)nc(C2(OC)CCOC2)c1] |
| | Pyridine: 2-chloro-4-(2-methoxypropoxy)-6-(3-methoxytetrahydrofuran-3- |
| | yl)pyridine (Preparation 114) |
| | Prep-HPLC-C (Gradient: 32-62% MeCN) |
| | (188.2 mg, 25.0% yield) as a white solid. LCMS m/z = 442.3 [M + H]⁺ |
| | 1H NMR (400 MHz, CDCl3) δ ppm: 9.35 (s, 1H), 8.77 (s, 1H), 8.21 (s, |
| | 1H), 8.17 (s, 1H), 7.49 (d, J = 2.4 Hz, 1H), 7.04 (d, J = 2.4 Hz, 1H), 4.28-4.08 |
| | (m, 6H), 3.79-3.78 (m, 1H), 3.48 (s, 3H), 3.30 (s, 3H), 2.98-2.94 (m, 1H), |
| | 2.57-2.52 (m, 1H), 2.25 (s, 3H), 1.31 (d, J = 6.8 Hz, 3H). |
| 11 | N-(1-(6-(3-methoxytetrahydrofuran-3-yl)-4-(2,2,2-trifluoroethoxy)pyridin- |
| | 2-yl)-1H-pyrazolo[4,3-c]pyridin-6-yl)acetamide |
| | 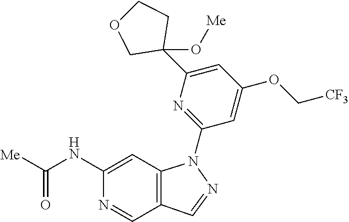 [SMILES: COC1(c2cc(OCC(F)(F)F)cc(-n3ncc4cnc(NC(C)=O)cc43)n2)CCOC1] |
| | Pyridine: 2-chloro-6-(3-methoxytetrahydrofuran-3-yl)-4-(2,2,2- |
| | trifluoroethoxy)pyridine (Preparation 101) |
| | Prep-HPLC-C (Gradient: 36-66% MeCN) |
| | 16.9 mg, 11.7% yield as white solid. LCMS m/z = 452.2 [M + H]⁺. 1H NMR: |
| | (400 MHz, CDCl₃) δ ppm: 9.35 (s, 1H), 8.78 (s, 1H), 8.21 (s, 1H), 8.04 (s, |
| | 1H), 7.52 (d, J = 2.0 Hz, 1H), 7.08 (d, J = 2.0 Hz, 1H), 4.57-4.51 (m, 2H), |
| | 4.29-4.22 (m, 3H), 4.10-4.08 (m, 1H), 3.32 (s, 3H), 3.02-2.96 (m, 1H), 2.59- |
| | 2.54 (m, 1H), 2.56 (s, 3H). |
| 12 | N-(1-(4-(3,3-difluorocyclobutoxy)-6-(3-methoxytetrahydrofuran-3- |
| | yl)pyridin-2-yl)-1H-pyrazolo[4,3-c]pyridin-6-yl)acetamide |
| | 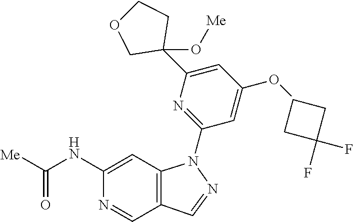 |
| | Pyridine: 2-chloro-4-(3,3-difluorocyclobutoxy)-6-(3- |
| | methoxytetrahydrofuran-3-yl)pyridine (Preparation 98) |
| | Prep-HPLC-C (Gradient: 37-67% MeCN) |
| | 11.5 mg, 25% as a yellow solid. LCMS m/z = 460.2 [M + H]⁺ |
| | 1H NMR: (500 MHz, CDCl₃) δ ppm: 9.34 (s, 1H), 8.78 (s, 1H), 8.21 (s, |
| | 1H), 8.12 (s, 1H), 7.36 (s, 1H), 6.95 (d, J = 2.0 Hz, 1H), 4.85-4.84 (m, 1H), |
| | 4.29-4.21 (m, 3H), 4.11-4.08 (m, 1H), 3.31 (s, 3H), 3.22-3.21 (m, 2H), 2.98- |
| | 2.96 (m, 1H), 2.87-2.83 (m, 2H), 2.55-2.52 (m, 1H), 2.26 (s, 3H). |
| 13 | N-(1-(4-methoxy-6-(3-methoxytetrahydrofuran-3-yl)pyridin-2-yl)-3-methyl- |
| | 1H-pyrazolo[4,3-c]pyridin-6-yl)acetamide |
| | 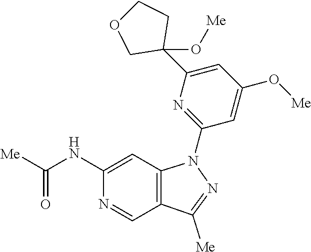 [SMILES: COc1cc(-n2nc(C)c3cnc(NC(C)=O)cc32)nc(C2(OC)CCOC2)c1] |
| | Pyridine: 2-chloro-4-methoxy-6-(3-methoxytetrahydrofuran-3-yl)pyridine |
| | (Preparation 91) |
| | Prep-HPLC-C (Gradient: 32-62% MeCN) |
| | 40 mg, 30.3% yield as a white solid. LCMS m/z = 398.2 [M + H]⁺ |
| | ¹H NMR: (500 MHz, CDCl₃) δ: ppm 9.28 (s, 1H), 8.67 (s, 1H), 8.11 (s, 1H), |
| | 7.44 (d, J = 2.5 Hz, 1H), 6.96 (d, J = 1.5 Hz, 1H), 4.29-4.20 (m, 3H), 4.11-4.08 |
| | (m, 1H), 3.97 (s, 3H), 3.30 (s, 3H), 2.98-2.95 (m, 1H), 2.66 (s, 3H), 2.56- |
| | 2.52 (m, 1H), 2.25 (s, 3H). |
| 14^{A} | N-(1-(4-ethoxy-6-(3-methoxytetrahydrofuran-3-yl)pyridin-2-yl)-3-methyl- |
| | 1H-pyrazolo[4,3-c]pyridin-6-yl)acetamide |
| | 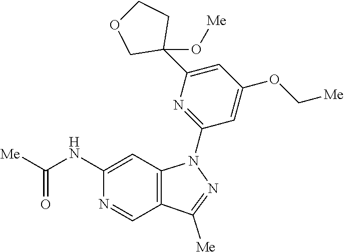 [SMILES: CCOc1cc(-n2nc(C)c3cnc(NC(C)=O)cc32)nc(C2(OC)CCOC2)c1] |
| | Pyridine: 2-chloro-4-ethoxy-6-(3-methoxytetrahydrofuran-3-yl)pyridine |
| | (Preparation 99) |
| | Prep-HPLC-F (Gradient: 38-53% MeCN) |
| | 25.4 mg, 23.5% as a white solid |
| | LCMS m/z = 412.2 [M + H]⁺. ¹H NMR: (500 MHz, CDCl₃) δ: ppm 9.28 (s, |
| | 1H), 8.66 (s, 1H), 8.13 (s, 1H), 7.42 (d, J = 2.0 Hz, 1H), 6.95 (d, J = 2.0 Hz, |
| | 1H), 4.28-4.20 (m, 5H), 4.11-4.08 (m, 1H), 3.30 (s, 3H), 2.98-2.94 (m, 1H), |
| | 2.65 (s, 3H), 2.57-2.51 (m, 1H), 2.25 (s, 3H), 1.48 (t, J = 6.5 Hz, 3H). |
| 15 | N-(1-(4-isopropoxy-6-(3-methoxytetrahydrofuran-3-yl)pyridin-2-yl)-3- |
| | methyl-1H-pyrazolo[4,3-c]pyridin-6-yl)acetamide |
| | 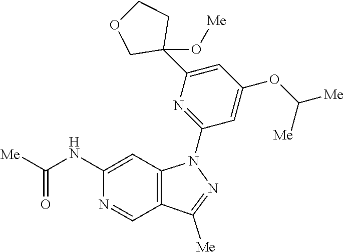 [SMILES: COC1(c2cc(OC(C)C)cc(-n3nc(C)c4cnc(NC(C)=O)cc43)n2)CCOC1] |
| | Pyridine: 2-chloro-4-isopropoxy-6-(3-methoxytetrahydrofuran-3-yl)pyridine |
| | (Preparation 100) |
| | Prep-HPLC-F (Gradient: 40-70% MeCN) |
| | 12.1 mg, 10.8% yield as a white solid. LCMS m/z = 426.2 [M + H]⁺ |
| | ¹H NMR: (500 MHz, CDCl₃) δ: ppm 9.27 (s, 1H), 8.66 (s, 1H), 8.22 (s, 1H), |
| | 7.40 (s, 1H), 6.92 (s, 1H), 4.81-4.80 (m, 1H), 4.26-4.08 (m, 4H), 3.30 (s, |
| | 3H), 2.96-2.95 (m, 1H), 2.64 (s, 3H), 2.54-2.53 (m, 1H), 2.24 (s, 3H), 1.43- |
| | 1.42 (m, 6H). |
| 16 | N-(1-(4-(2-methoxyethoxy)-6-(3-methoxytetrahydrofuran-3-yl)pyridin-2- |
| | yl)-3-methyl-1H-pyrazolo[4,3-c]pyridin-6-yl)acetamide |
| | 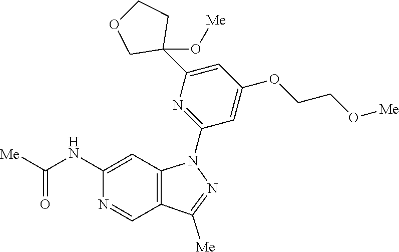 [SMILES: COCCOc1cc(-n2nc(C)c3cnc(NC(C)=O)cc32)nc(C2(OC)CCOC2)c1] |
| | Pyridine: 2-chloro-4-(2-methoxyethoxy)-6-(3-methoxytetrahydrofuran-3- |
| | yl)pyridine (Preparation 115) |
| | Prep-HPLC-F (Gradient: 30-60% MeCN) |
| | 57.6 mg, 49.6% yield as a white solid. LCMS m/z = 442.3 [M + H]⁺ |
| | ¹H NMR: (400 MHz, CDCl₃) δ: ppm 9.28 (s, 1H), 8.66 (s, 1H), 8.10 (s, 1H), |
| | 7.46 (d, J = 2.4 Hz, 1H), 7.01 (d, J = 2.4 Hz, 1H), 4.32-4.21 (m, 5H), 4.10-4.06 |
| | (m, 1H), 3.83-3.80 (m, 2H), 3.48 (s, 3H), 3.28 (s, 3H), 2.98-2.94 (m, 1H), |
| | 2.65 (s, 3H), 2.56-2.53 (m, 1H), 2.24 (s, 3H). |
| 17 | N-(1-(4-(2-methoxypropoxy)-6-(3-methoxytetrahydrofuran-3-yl)pyridin-2- |
| | yl)-3-methyl-1H-pyrazolo[4,3-c]pyridin-6-yl)acetamide |
| | 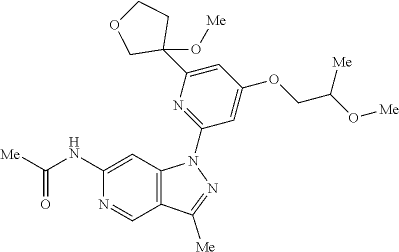 [SMILES: COC(C)COc1cc(-n2nc(C)c3cnc(NC(C)=O)cc32)nc(C2(OC)CCOC2)c1] |
| | Pyridine: 2-chloro-4-(2-methoxypropoxy)-6-(3-methoxytetrahydrofuran-3- |
| | yl)pyridine (Preparation 114) |
| | Prep-HPLC-F (Gradient: 32-62% MeCN) |
| | 6.4 mg, 8.9% yield as a white solid. LCMS m/z = 456.2 [M + H]⁺ |
| | ¹H NMR: (500 MHz, CDCl₃) δ: ppm 9.28 (s, 1H), 8.66 (s, 1H), 8.10 (s, 1H), |
| | 7.44 (d, J = 2.0 Hz, 1H), 6.98 (d, J = 2.0 Hz, 1H), 4.21-4.27 (m, 3H), 4.14-4.07 |
| | (m, 3H), 3.79-3.77 (m, 1H), 3.48 (s, 3H), 3.29 (s, 3H), 2.97-2.94 (m, 1H), |
| | 2.65 (s, 3H), 2.56-2.54 (m, 1H), 2.24 (s, 3H), 1.32 (d, J = 6.0 Hz, 3H). |

A-compound was further purified by prep TLC (EtOAc)

### Example 18 and 19: (S)—N-(1-(4-(3-(difluoromethyl)cyclobutoxy)-6-(3-methoxytetrahydrofuran-3-yl)pyridin-2-yl)-1H-pyrazolo[4,3-c]pyridin-6-yl)acetamide and (R)—N-(1-(4-(3-(difluoromethyl)cyclobutoxy)-6-(3-methoxytetrahydrofuran-3-yl)pyridin-2-yl)-1H-pyrazolo[4,3-c]pyridin-6-yl)acetamide

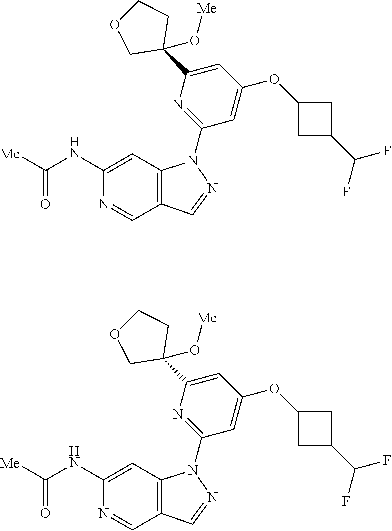 [SMILES: CO[C@@]1(c2cc(OC3CC(C(F)F)C3)cc(-n3ncc4cnc(NC(C)=O)cc43)n2)CCOC1 ; CO[C@]1(c2cc(OC3CC(C(F)F)C3)cc(-n3ncc4cnc(NC(C)=O)cc43)n2)CCOC1]

To a solution of 2-chloro-4-(3-(difluoromethyl)cyclobutoxy)-6-(3-methoxytetrahydrofuran-3-yl)pyridine (Preparation 95, 220 mg, 0.659 mmol) and N-(1H-pyrazolo[4,3-c]pyridin-6-yl)acetamide (Preparation 2, 116.1 mg, 0.659 mmol) in dioxane (5 mL) was added Pd₂(dba)₃ (60.4 mg, 0.066 mmol), XantPhos (76.3 mg, 0.132 mmol) and Zn(OAc)₂ (60.5 mg, 0.330 mmol) and the reaction mixture was stirred at 120° C. for 12 h under N₂. The cooled mixture was concentrated under reduced pressure and the residue was purified by Prep-HPLC-F (Gradient: 40-70% MeCN) to give N-(1-(4-(3-(difluoromethyl)cyclobutoxy)-6-(3-methoxytetrahydrofuran-3-yl)pyridin-2-yl)-1H-pyrazolo[4,3-c]pyridin-6-yl)acetamide (92 mg, 29.5% yield) as a white solid. LCMS m/z=474.3 [M+H]⁺

This was further purified by SFC (Instrument: SFC-21; Condition: 0.1% NH₃·H₂O IPA; Column: DAICEL CHIRALCEL OD-H (250 mm*30 mm,5 um); Begin B: 45%; End B: 45%; FlowRate (ml/min): 60) to give

Peak 1, enantiomer 1 (26.8 mg, 29.1% yield) as a white solid. LCMS m/z=474.3 [M+H]⁺ 1H NMR (400 MHz, CDCl₃) δ ppm: 9.35 (s, 1H), 8.77 (s, 1H), 8.21-8.20 (m, 2H), 7.32 (d, J=2.0 Hz, 1H), 6.92 (d, J=2.0 Hz, 1H), 5.96 (t, J=57.2 Hz, 1H), 5.00-4.97 (m, 1H), 4.28-4.21 (m, 3H), 4.10-4.08 (m, 1H), 3.31 (s, 3H), 2.99-2.96 (m, 1H), 2.71-2.68 (m, 3H), 2.44-2.41 (m, 3H), 2.25 (s, 3H) and

Peak 2, enantiomer 2 (29.2 mg, 31.7% yield) as a white solid. LCMS m/z=474.3 [M+H]⁺ 1H NMR: (400 MHz, CDCl₃) δ ppm: 9.34 (s, 1H), 8.77 (s, 1H), 8.21 (d, J=0.8 Hz, 1H), 8.09 (s, 1H), 7.32 (d, J=2.4 Hz, 1H), 6.92 (d, J=2.0 Hz, 1H), 5.96 (t, J=57.2 Hz, 1H), 4.98-4.93 (m, 1H), 4.28-4.21 (m, 3H), 4.10-4.08 (m, 1H), 3.31 (s, 3H), 2.99-2.96 (m, 1H), 2.71-2.68 (m, 3H), 2.46-2.44 (m, 3H), 2.25 (s, 3H).

### Example 20: N-(1-(4-(3-hydroxycyclobutoxy)-6-(3-methoxytetrahydrofuran-3-yl)pyridin-2-yl)-1H-pyrazolo[4,3-c]pyridin-6-yl)acetamide

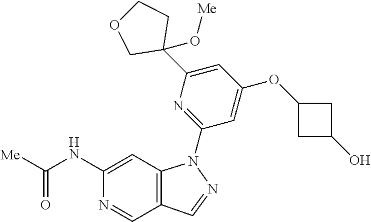 [SMILES: COC1(c2cc(OC3CC(O)C3)cc(-n3ncc4cnc(NC(C)=O)cc43)n2)CCOC1]

Step A: To a solution of 4-(3-(benzyloxy)cyclobutoxy)-2-chloro-6-(3-methoxytetrahydrofuran-3-yl)pyridine (Preparation 97, 500 mg, 1.28 mmol) and N-(1H-pyrazolo[4,3-c]pyridin-6-yl)acetamide (Preparation 2, 225.9 mg, 1.28 mmol) in dioxane (8.0 mL) was added Pd₂(dba)₃ (117.4 mg, 0.128 mmol), Xantphos (148.4 mg, 0.256 mmol) and Zn(OAc)₂ (352.9 mg, 1.92 mmol) at 25° C. The mixture was stirred at 120° C. for 16 hours under N₂. The mixture was poured into H₂O (20 mL), extracted with EtOAc (3×20 mL), the combined organic layer was dried over anhydrous Na₂SO₄, filtered and concentrated. The residue was purified by chromatography (EtOAc=100%) on silica gel to give N-(1-(4-(3-(benzyloxy)cyclobutoxy)-6-(3-methoxytetrahydrofuran-3-yl)pyridin-2-yl)-1H-pyrazolo[4,3-c]pyridin-6-yl)acetamide (209.8 mg, 30.9% yield) as colorless oil. ¹H NMR: (400 MHz, CDCl₃) δ ppm: 9.35 (s, 1H), 8.75 (s, 1H), 8.45 (s, 1H), 8.20 (s, 1H), 7.29-7.38 (m, 6H), 6.91 (d, J=2.0 Hz, 1H), 5.04-5.08 (m, 1H), 4.47 (s, 2H), 4.20-4.40 (m, 5H), 3.30 (s, 3H), 2.94-2.98 (m, 1H), 2.59-2.63 (m, 2H), 2.49-2.55 (m, 3H), 2.25 (s, 3H).

Step B: A solution of N-(1-(4-(3-(benzyloxy)cyclobutoxy)-6-(3-methoxytetrahydrofuran-3-yl)pyridin-2-yl)-1H-pyrazolo[4,3-c]pyridin-6-yl)acetamide (180 mg, 0.340 mmol) in TFA (3.0 g, 26.1 mmol) was stirred at 100° C. for 4 h. The mixture was purified by Prep-HPLC-C (Gradient: 21-51% MeCN) to give N-(1-(4-(3-hydroxycyclobutoxy)-6-(3-methoxytetrahydrofuran-3-yl)pyridin-2-yl)-1H-pyrazolo[4,3-c]pyridin-6-yl)acetamide (24.8 mg, 16.6% yield) as a white solid. LCMS m/z=440.3 [M+H]⁺. ¹H NMR: (500 MHz, DMSO-d₆) δ ppm: 10.68 (s, 1H), 9.27 (s, 1H), 8.95 (s, 1H), 8.54 (s, 1H), 7.28 (d, J=2.0 Hz, 1H), 6.88 (s, 1H), 5.29-5.27 (m, 1H), 5.09-5.06 (m, 1H), 4.40-4.22 (m, 1H), 4.21-4.20 (m, 1H), 4.08-3.97 (m, 2H), 3.90-3.87 (m, 1H), 3.31-3.30 (m, 1H), 3.15 (s, 3H), 2.76-2.73 (m, 1H), 2.42-2.36 (m, 4H), 2.15 (s, 3H).

### Example 21: N-(1-(4-(3-methoxycyclobutoxy)-6-(3-methoxytetrahydrofuran-3-yl)pyridin-2-yl)-1H-pyrazolo[4,3-c]pyridin-6-yl)acetamide

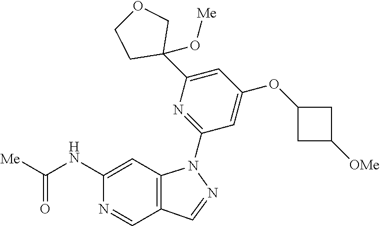 [SMILES: COC1CC(Oc2cc(-n3ncc4cnc(NC(C)=O)cc43)nc(C3(OC)CCOC3)c2)C1]

To a solution of 2-chloro-4-(3-methoxycyclobutoxy)-6-(3-methoxytetrahydrofuran-3-yl)pyridine (Preparation 103, 50 mg, 0.159 mmol) in dioxane (3 mL) was added N-(1H-pyrazolo[4,3-c]pyridin-6-yl)acetamide (Preparation 2, 56.2 mg, 0.319 mmol), Pd(OAc)₂ (3.6 mg, 15.9 umol), XantPhos (18.4 mg, 31.9 umol) and t-BuONa (30.6 mg, 0.319 mmol) and the mixture was stirred at 130° C. under microwave irradiation and N₂ for 2 h. The mixture was purified by Prep-HPLC-C (Gradient: 32-62% MeCN) to give N-(1-(4-(3-methoxycyclobutoxy)-6-(3-methoxytetrahydrofuran-3-yl)pyridin-2-yl)-1H-pyrazolo[4,3-c]pyridin-6-yl)acetamide (12.4 mg, 17.2% yield) as a white solid. LCMS m/z=454.2 [M+H]⁺. ¹H NMR: (400 MHz, CDCl₃) δ ppm: 9.33 (s, 1H), 8.77 (s, 1H), 8.20-8.17 (m, 2H), 7.34 (s, 1H), 6.92 (s, 1H), 5.03-5.02 (m, 1H), 4.28-4.08 (m, 5H), 3.30 (s, 6H), 2.98-2.94 (m, 1H), 2.56-2.48 (m, 5H), 2.25 (s, 3H).

### Example 22: N-(1-(4-(2-methoxypropoxy)-6-(tetrahydrofuran-3-yl)pyridin-2-yl)-3-methyl-1H-pyrazolo[4,3-c]pyridin-6-yl)acetamide

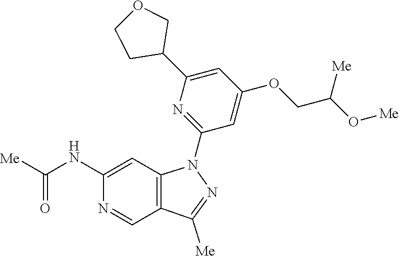 [SMILES: COC(C)COc1cc(C2CCOC2)nc(-n2nc(C)c3cnc(NC(C)=O)cc32)c1]

Step A: N-(1-(6-(Furan-3-yl)-4-(2-methoxypropoxy)pyridin-2-yl)-3-methyl-1H-pyrazolo[4,3-c]pyridin-6-yl)acetamide was obtained as a yellow solid, 70 mg, 31.6%, from N-(1H-pyrazolo[4,3-c]pyridin-6-yl)acetamide (Preparation 2), and 2-chloro-6-(furan-3-yl)-4-(2-methoxypropoxy)pyridine (Preparation 126) following the procedure described in Example 20, step A. LCMS m/z=422.1 [M+H]⁺

Step B: To a solution of N-(1-(6-(furan-3-yl)-4-(2-methoxypropoxy)pyridin-2-yl)-3-methyl-1H-pyrazolo[4,3-c]pyridin-6-yl)acetamide (60.0 mg, 0.142 mmol) in MeOH (5 mL) was added Pd/C (151.5 mg, 0.142 mmol, 10% purity) and the mixture was stirred at 25° C. for 2 h under 15 psi of H₂. The mixture was filtered and the organic layer was concentrated to give crude product. This was purified by Prep-HPLC-C (Gradient: 33-63% MeCN) to give N-(1-(4-(2-methoxypropoxy)-6-(tetrahydrofuran-3-yl)pyridin-2-yl)-3-methyl-1H-pyrazolo[4,3-c]pyridin-6-yl)acetamide (3.0 mg, 5.0% yield) as white solid. LCMS m/z=426.2 [M+H]⁺. ¹H NMR: (500 MHz, CDCl₃) δ ppm: 9.39 (s, 1H), 8.66 (s, 1H), 8.07 (s, 1H), 7.35 (d, J=2.0 Hz, 1H), 6.66 (d, J=2.0 Hz, 1H), 4.31-4.27 (m, 1H), 4.23-4.21 (m, 1H), 4.14-4.10 (m, 1H), 4.08-4.04 (m, 1H), 4.03-4.02 (m, 2H), 4.01-3.98 (m, 1H), 3.61-3.58 (m, 1H), 3.47 (s, 3H), 2.65 (s, 3H), 2.51-2.44 (m, 2H), 2.25 (s, 3H), 1.30 (d, J=6.5 Hz, 3H).

### Example 23: N-(1-(6-(1,1-difluoroethyl)-4-methoxypyridin-2-yl)-3-methyl-1H-pyrazolo[4,3-c]pyridin-6-yl)acetamide

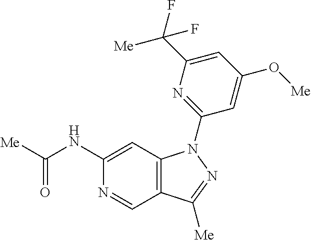 [SMILES: COc1cc(-n2nc(C)c3cnc(NC(C)=O)cc32)nc(C(C)(F)F)c1]

To a solution of 2-chloro-6-(1,1-difluoroethyl)-4-methoxypyridine (Preparation 133, 50 mg, 0.241 mmol) and N-(3-methyl-1H-pyrazolo[4,3-c]pyridin-6-yl)acetamide (Preparation 6, 45.8 mg, 0.241 mmol) in dioxane (3.0 mL) was added BrettPhos Pd G3 (43.7 mg, 0.048 mmol) and Cs₂CO₃ (156.9 mg, 0.482 mmol) and the reaction was stirred at 100° C. for 2 h under N₂. The crude was purified by Prep-HPLC-C (Gradient: 40-70% MeCN) to give N-(1-(6-(1,1-difluoroethyl)-4-methoxypyridin-2-yl)-3-methyl-1H-pyrazolo[4,3-c]pyridin-6-yl)acetamide (24.7 mg, 28.4% yield) as a white solid. LCMS m/z=362.1 [M+H]⁺. ¹H NMR: (500 MHz, CDCl₃) δ ppm: 9.41 (s, 1H), 8.68 (s, 1H), 8.11 (s, 1H), 7.55 (s, 1H), 7.09 (d, J=2.0 Hz, 1H), 4.00 (s, 3H), 2.67 (s, 3H), 2.67-2.26 (m, 6H).

### Examples 24 to 31

The Examples in the following table were prepared from the appropriate halopyridine and N-(1H-pyrazolo[4,3-c]pyridin-6-yl)acetamide (Preparation 2), following a similar procedure to that described in Example 23.

| | |
|---|---|
| Example No | Name, Structure, Starting Materials, Data |

Using N-(3-methyl-1H-pyrazolo[4,3-c]pyridin-6-yl)acetamide (Preparation 6)

| | |
|---|---|
| 24 | N-(1-(6-(1,1-difluoroethyl)-4-(2-methoxyethoxy)pyridin-2-yl)-3-methyl- |
| | 1H-pyrazolo[4,3-c]pyridin-6-yl)acetamide |
| | 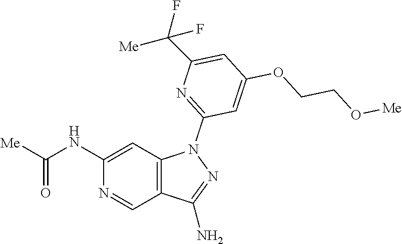 [SMILES: COCCOc1cc(-n2nc(N)c3cnc(NC(C)=O)cc32)nc(C(C)(F)F)c1] |
| | Pyridine: 2-chloro-6-(1,1-difluoroethyl)-4-(2-methoxyethoxy)pyridine |
| | (Preparation 117) |
| | Prep-HPLC-C (Gradient: 37-67% MeCN) |
| | 11.6 mg, 36% yield as a white solid. LCMS m/z = 406.2 [M + H]⁺. 1H |
| | NMR: (400 MHz, CDCl₃) δ ppm: 9.39 (s, 1H), 8.67 (s, 1H), 8.11 (s, 1H), |
| | 7.56 (s, 1H), 7.12 (d, J = 1.6 Hz, 1H), 4.33-4.30 (m, 2H), 3.82-3.80 (m, |
| | 2H), 3.47 (s, 3H), 2.65 (s, 3H), 2.33-2.23 (m, 6H). |

Using N-(3-ethyl-1H-pyrazolo[4,3-c]pyridin-6-yl)acetamide (Preparation 9)

| | |
|---|---|
| 25 | N-(1-(6-(1,1-difluoroethyl)-4-(2-methoxyethoxy)pyridin-2-yl)-3-ethyl-1H- |
| | pyrazolo[4,3-c]pyridin-6-yl)acetamide |
| | 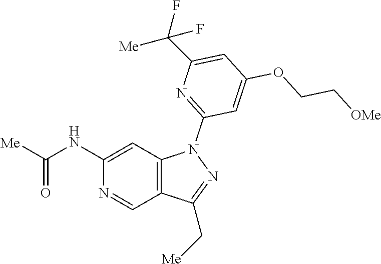 [SMILES: CCc1nn(-c2cc(OCCOC)cc(C(C)(F)F)n2)c2cc(NC(C)=O)ncc12] |
| | pyridine: 2-chloro-6-(1,1-difluoroethyl)-4-(2-methoxyethoxy)pyridine |
| | (Preparation 117) |
| | Prep-HPLC-C (Gradient: 42-72% MeCN) |
| | 25.0 mg, 33.3%. LCMS m/z = 420.1 [M + H]⁺. ¹H NMR: (500 MHz, |
| | CDCl₃) δ ppm: 9.40 (s, 1H), 8.70 (s, 1H), 8.02 (s, 1H), 7.59 (d, J = 2.0 Hz, |
| | 1H), 7.12 (d, J = 2.0 Hz, 1H), 4.31 (t, J = 4.5 Hz, 2H), 3.81 (t, J = 4.5 Hz, |
| | 2H), 3.48 (s, 3H), 3.08-3.03 (m, 2H), 2.32-2.25 (m, 6H), 1.46 (t, J = 8.0 |
| | Hz, 3H). |
| 26 | N-(1-(6-(1,1-difluoroethyl)-4-(3-methoxycyclobutoxy)pyridin-2-yl)-3- |
| | ethyl-1H-pyrazolo[4,3-c]pyridin-6-yl)acetamide |
| | 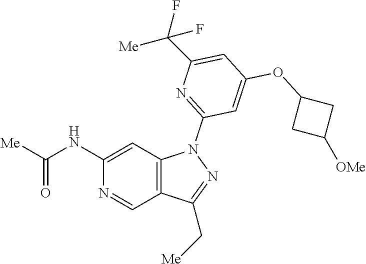 [SMILES: CCc1nn(-c2cc(OC3CC(OC)C3)cc(C(C)(F)F)n2)c2cc(NC(C)=O)ncc12] |
| | Pyridine: 2-chloro-6-(1,1-difluoroethyl)-4-(3- |
| | methoxycyclobutoxy)pyridine (Preparation 112) |
| | Prep-HPLC-C (Gradient: 50-80% MeCN) |
| | 5.4 mg, 16.8% yield as a white solid. LCMS m/z = 446.2 [M + H]⁺ 1H NMR: |
| | (400 MHz, CDCl₃) δ ppm: 9.40 (s, 1H), 8.71 (s, 1H), 8.17 (s, 1H), 7.43 |
| | (s, 1H), 6.99 (s, 1H), 5.08-5.00 (m, 1H), 4.19-4.15 (m, 1H), 3.30 (s, 3H), |
| | 3.09-3.03 (m, 2H), 2.56-2.50 (m, 4H), 2.33-2.23 (m, 6H), 1.47 (t, J = 7.2 |
| | Hz, 3H). |

Using N-(3-cyclopropyl-1H-pyrazolo[4,3-c]pyridin-6-yl)acetamide (Preparation 11)

| | |
|---|---|
| 27 | N-(3-cyclopropyl-1-(6-(1,1-difluoroethyl)-4-(3- |
| | methoxycyclobutoxy)pyridin-2-yl)-1H-pyrazolo[4,3-c]pyridin-6- |
| | yl)acetamide |
| | 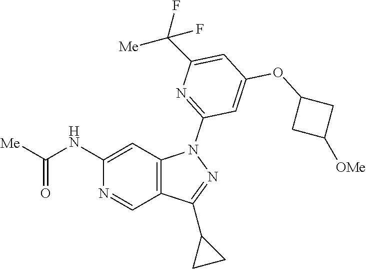 [SMILES: COC1CC(Oc2cc(-n3nc(C4CC4)c4cnc(NC(C)=O)cc43)nc(C(C)(F)F)c2)C1] |
| | Pyridine: 2-chloro-6-(1,1-difluoroethyl)-4-(3- |
| | methoxycyclobutoxy)pyridine (Preparation 112) |
| | prep-HPLC (Column: Agela DuraShell NH2 150 mm*30 mm*5 um; |
| | Condition: Heptane-EtOH (0.1% NH3•H2O); Begin B; 0; End B: 80; |
| | Gradient Time (min): 9; 100% B Hold Time (min): 2; Flow Rate |
| | (ml/min): 25; 48.6 mg, 36.9% yield as white solid. LCMS m/z = 458.2 |
| | [M + H]+ 1H NMR: (400 MHz, CDCl₃) δ ppm: 9.42 (s, 1H), 8.72 (s, 1H), |
| | 8.50 (s, 1H), 7.37 (s, 1H), 6.97 (d, J = 2.0 Hz, 1H), 5.06-5.00 (m, 1H), |
| | 4.19-4.13 (m, 1H), 3.30 (s, 3H), 2.57-2.48 (m, 4H), 2.32-2.22 (m, 7H), |
| | 1.21-1.16 (m, 4H). |
| 28 | N-(3-cyclopropyl-1-(6-(1,1-difluoroethyl)-4-isopropoxypyridin-2-yl)-1H- |
| | pyrazolo[4,3-c]pyridin-6-yl)acetamide |
| | 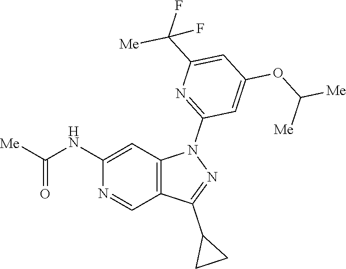 [SMILES: CC(=O)Nc1cc2c(cn1)c(C1CC1)nn2-c1cc(OC(C)C)cc(C(C)(F)F)n1] |
| | Pyridine: 2-chloro-6-(1,1-difluoroethyl)-4-isopropoxypyridine |
| | (Preparation 129) |
| | Prep-HPLC-F (Gradient: 57-87% MeCN) |
| | 15.1 mg, 13.8% yield as a white solid. LCMS m/z = 416.2 [M + H]⁺ |
| | 1H NMR: (500 MHz, CDCl₃) δ ppm: 9.41 (s, 1H), 8.73 (s, 1H), 8.28 (s, |
| | 1H), 7.45 (d, J = 2.0 Hz, 1H), 7.03 (d, J = 2.0 Hz, 1H), 4.83-4.77 (m, 1H), |
| | 2.32-2.28 (m, 1H), 2.27-2.24 (m, 6H), 1.42 (d, J = 6.0 Hz, 6H), 1.22-1.20 |
| | (m, 2H), 1.18-1.15 (m, 2H). |
| 29 | N-(3-cyclopropyl-1-(6-(1,1-difluoroethyl)-4-methoxypyridin-2-yl)-1H- |
| | pyrazolo[4,3-c]pyridin-6-yl)acetamide |
| | 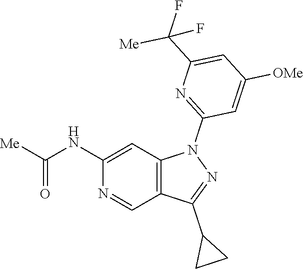 [SMILES: COc1cc(-n2nc(C3CC3)c3cnc(NC(C)=O)cc32)nc(C(C)(F)F)c1] |
| | Pyridine: 2-chloro-6-(1,1-difluoroethyl)-4-methoxypyridine (Preparation |
| | 133). |
| | Prep-HPLC-F (Gradient: 51-81% MeCN) |
| | a white solid, 12.8 mg, 19.4% yield. LCMS m/z = 384.2 [M + H]+. 1H |
| | NMR: (400 MHz, CDCl3) δ ppm: 9.37 (s, 1H), 8.72 (s, 1H), 8.04 (s, 1H), |
| | 7.34 (d, J = 2.0 Hz, 1H), 6.97 (s, 1H), 3.96 (s, 3H), 2.32-2.28 (m, 1H), 2.26 |
| | (s, 3H), 1.92 (s, 3H), 1.86 (s, 3H), 1.22-1.20 (m, 2H), 1.17-1.14 (m, 2H). |
| 30 | N-(3-cyclopropyl-1-(6-(2-oxopyrrolidin-1-yl)pyridin-2-yl)-1H- |
| | pyrazolo[4,3-c]pyridin-6-yl)acetamide |
| | 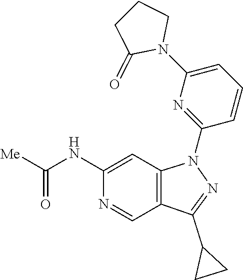 [SMILES: CC(=O)Nc1cc2c(cn1)c(C1CC1)nn2-c1cccc(N2CCCC2=O)n1] |
| | Pyridine: 1-(6-chloropyridin-2-yl)pyrrolidin-2-one (Preparation 88) |
| | Prep-HPLC-F (Gradient: 47-77% MeCN) white solid, 24.6 mg, 18.4% |
| | yield, LCMS m/z = 377.2 [M + H]⁺. 1H NMR: (500 MHz, DMSO-d₆) δ: |
| | ppm 10.69 (s, 1H), 9.22 (s, 1H), 8.95 (s, 1H), 8.5 (d, J = 8.5 Hz, 1H), 7.94 |
| | (t, J = 8.0 Hz, 1H), 7.58 (d, J = 8.0 Hz, 1H), 4.31 (t, J = 7.0 Hz, 2H), 2.65- |
| | 2.50 (m, 2H), 2.52-2.49 (m, 1H), 2.15 (s, 3H), 2.14-2.10 (m, 2H), 1.16- |
| | 1.13 (m, 4H). |
| 31 | N-(3-cyclopropyl-1-(4-(1,1-difluoroethyl)pyridin-2-yl)-1H-pyrrolo[3,2- |
| | c]pyridin-6-yl)acetamide |
| | 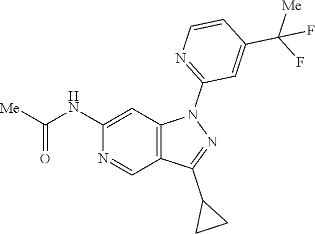 [SMILES: CC(=O)Nc1cc2c(cn1)c(C1CC1)nn2-c1cc(C(C)(F)F)ccn1] |
| | N-(3-cyclopropyl-1H-pyrrolo[3,2-c]pyridin-6-yl)acetamide (Preparation |
| | 21) and 2-chloro-4-(2-fluoropropan-2-yl)pyridine (Preparation 128) |
| | Prep-HPLC-C (Gradient: 41-71% MeCN) |
| | 12.0 mg, 17.1% yield as a yellow solid. LCMS m/z = 357.1 [M + H]⁺ 1H |
| | NMR: (500 MHz, MeOH-d₄) δ ppm: 8.96 (s, 1H), 8.73-8.62 (m, 2H), |
| | 7.78-7.73 (m, 1H), 7.67 (s, 1H), 7.46-7.41 (m, 1H), 2.21 (s, 3H), 2.07- |
| | 1.98 (m, 4H), 1.02-0.98 (m, 2H), 0.80-0.77 (m, 2H). |

### Example 32: N-(3-cyclopropyl-1-(6-(1,1-difluoroethyl)-4-(2-methoxyethoxy)pyridin-2-yl)-1H-pyrazolo[4,3-c]pyridin-6-yl)acetamide

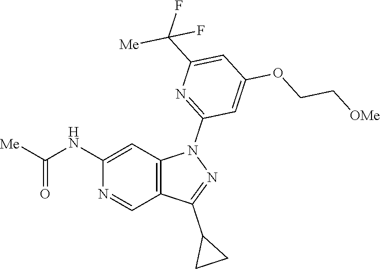 [SMILES: COCCOc1cc(-n2nc(C3CC3)c3cnc(NC(C)=O)cc32)nc(C(C)(F)F)c1]

To a solution of 2-chloro-6-(1,1-difluoroethyl)-4-(2-methoxyethoxy)pyridine (Preparation 117, 75.0 mg, 0.298 mmol) in dioxane (8 mL) was added N-(3-cyclopropyl-1H-pyrazolo[4,3-c]pyridin-6-yl)acetamide (Preparation 11, 64.4 mg, 0.298 mmol), Cs₂CO₃ (194.2 mg, 0.596 mmol) and BrettPhos Pd G₃ (54.0 mg, 0.060 mmol) and the reaction was stirred at 100° C. for 16 h under N₂. The reaction was treated with H₂O (10 mL) and extracted with EtOAc (5 mL×3). The organic phase was washed with brine (15 mL), dried over Na₂SO₄, filtered and concentrated in vacuo. The crude was purified by column chromatography (PE/EtOAc=3/1 to 0/1) on silica gel to give product. The residue was purified by Prep-HPLC-C (Gradient: 43-73% MeCN) to give N-(3-cyclopropyl-1-(6-(1,1-difluoroethyl)-4-(2-methoxyethoxy)pyridin-2-yl)-1H-pyrazolo[4,3-c]pyridin-6-yl)acetamide, 20.8 mg, 16.2% yield as white solid. LCMS m/z=432.1 [M+H]⁺. ¹H NMR: (400 MHz, CDCl₃) δ ppm: 9.48 (s, 1H), 8.80 (s, 1H), 8.72 (s, 1H), 7.54 (s, 1H), 7.12 (d, J=1.6 Hz, 1H), 4.32-4.30 (m, 2H), 3.83-3.80 (m, 2H), 3.48 (s, 3H), 2.32-2.22 (m, 7H), 1.22-1.17 (m, 4H)

### Example 33: N-(3-ethyl-1-(4-(3-methoxycyclobutoxy)-6-(tetrahydrofuran-3-yl)pyridin-2-yl)-1H-pyrazolo[4,3-c]pyridin-6-yl)acetamide

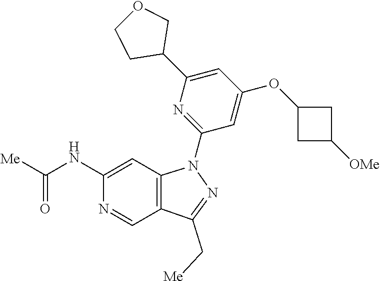 [SMILES: CCc1nn(-c2cc(OC3CC(OC)C3)cc(C3CCOC3)n2)c2cc(NC(C)=O)ncc12]

Step A: To a solution of 2-chloro-6-(furan-3-yl)-4-(3-methoxycyclobutoxy)pyridine (Preparation 107, 250 mg, 0.894 mmol) in dioxane (3 mL) was added N-(3-ethyl-1H-pyrazolo[4,3-c]pyridin-6-yl)acetamide (Preparation 9, 182.5 mg, 0.894 mmol), Cs₂CO₃ (582.4 mg, 1.79 mmol) and BrettPhos Pd G₃ (162.0 mg, 0.179 mmol) and the reaction was stirred at 100° C. for 16 h under N₂. The mixture was concentrated and purified by Prep-HPLC-F (Gradient: 54-84% MeCN) to give N-(3-ethyl-1-(6-(furan-3-yl)-4-(3-methoxycyclobutoxy)pyridin-2-yl)-1H-pyrazolo[4,3-c]pyridin-6-yl)acetamide (46 mg, 11.5% yield) as a white solid. ¹H NMR: (400 MHz, CDCl₃) δ ppm: 10.69 (s, 1H), 9.90 (s, 1H), 8.68 (s, 1H), 8.55 (s, 1H), 7.57 (s, 1H), 7.30 (d, J=2.0 Hz, 1H), 7.04 (s, 1H), 6.84 (d, J=1.6 Hz, 1H), 5.08-5.04 (m, 1H), 4.21-4.17 (m, 1H), 3.32 (s, 3H), 3.10 (q, J=7.6 Hz, 2H), 2.57-2.51 (m, 4H), 2.38 (s, 3H), 1.50 (t, J=7.6 Hz, 3H).

Step B: To a solution of N-(3-ethyl-1-(6-(furan-3-yl)-4-(3-methoxycyclobutoxy)pyridin-2-yl)-1H-pyrazolo[4,3-c]pyridin-6-yl)acetamide (41 mg, 0.092 mmol) in THF (4 mL) was added Pd/C (9.8 mg, 9.16 umol, 10% purity) and the reaction mixture was stirred at 25° C. for 16 h under 30 psi of H₂. The mixture was concentrated and was purified by Prep-HPLC-F (Gradient: 40-70% MeCN) to give N-(3-ethyl-1-(4-(3-methoxycyclobutoxy)-6-(tetrahydrofuran-3-yl)pyridin-2-yl)-1H-pyrazolo[4,3-c]pyridin-6-yl)acetamide (8.7 mg, 21.0% yield) as a white solid. LCMS m/z=452.2 [M+H]¹H NMR: (400 MHz, CDCl₃) δ ppm: 9.38 (s, 1H), 8.69 (s, 1H), 8.05 (s, 1H), 7.22 (d, J=2.0 Hz, 1H), 6.53 (d, J=2.0 Hz, 1H), 5.03-5.00 (m, 1H), 4.29-4.16 (m, 3H), 4.03-4.00 (m, 2H), 3.61-3.58 (m, 1H), 3.30 (s, 3H), 3.08-3.04 (m, 2H), 2.54-2.48 (m, 6H), 2.25 (s, 3H), 1.47 (t, J=7.6 Hz, 3H).

### Example 34: N-(3-cyclopropyl-1-(4-(3-methoxycyclobutoxy)-6-(tetrahydrofuran-3-yl)pyridin-2-yl)-1H-pyrazolo[4,3-c]pyridin-6-yl)acetamide

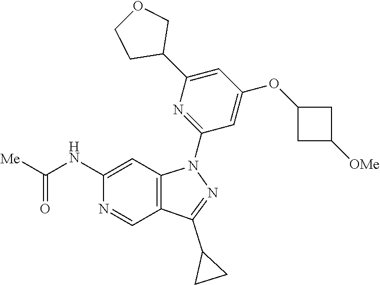 [SMILES: COC1CC(Oc2cc(C3CCOC3)nc(-n3nc(C4CC4)c4cnc(NC(C)=O)cc43)c2)C1]

N-(3-Cyclopropyl-1-(4-(3-methoxycyclobutoxy)-6-(tetrahydrofuran-3-yl)pyridin-2-yl)-1H-pyrazolo[4,3-c]pyridin-6-yl)acetamide was obtained from 2-chloro-6-(furan-3-yl)-4-(3-methoxycyclobutoxy)pyridine (Preparation 107) and N-(3-cyclopropyl-1H-pyrazolo[4,3-c]pyridin-6-yl)acetamide (Preparation 11) following a similar procedure to that described in Example 33. LCMS m/z=464.2 [M+H]¹H NMR: (500 MHz, CDCl₃) δ ppm: 11.34 (s, 1H), 9.80 (s, 1H), 8.69 (s, 1H), 7.19 (d, J=2.0 Hz, 1H), 6.61 (s, 1H), 5.02-4.99 (m, 1H), 4.29-4.27 (m, 1H), 4.26-4.25 (m, 2H), 4.01-3.94 (m, 2H), 3.63-3.60 (m, 1H), 3.30 (s, 3H), 2.55-2.53 (m, 2H), 2.48-2.46 (m, 4H), 2.44 (s, 3H), 2.38-2.37 (m, 1H), 1.27-1.23 (m, 4H).

### Example 35: N-(1-(4-(2-methoxyethoxy)-6-(tetrahydrofuran-3-yl)pyridin-2-yl)-3-methyl-1H-pyrazolo[4,3-c]pyridin-6-yl)acetamide

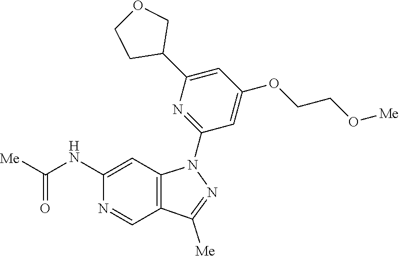 [SMILES: COCCOc1cc(C2CCOC2)nc(-n2nc(C)c3cnc(NC(C)=O)cc32)c1]

Step A: To a solution of 2-chloro-6-(furan-3-yl)-4-(2-methoxyethoxy)pyridine (Preparation 136, 109.1 mg, 0.573 mmol), N-(3-methyl-1H-pyrazolo[4,3-c]pyridin-6-yl)acetamide (Preparation 6, 160 mg, 0.631 mmol) and BrettPhos Pd G3 (104 mg, 0.115 mmol) in dioxane (15 mL) was added Cs₂CO₃ (373.6 mg, 1.15 mmol) and the reaction was stirred at 100° C. for 3 h at N₂. The mixture was concentrated in vacuo and the crude was purified by silica gel column chromatography (PE/EtOAc=1/2) to give N-(1-(6-(furan-3-yl)-4-(2-methoxyethoxy)pyridin-2-yl)-3-methyl-1H-pyrazolo[4,3-c]pyridin-6-yl)acetamide (100 mg, 42.8% yield) as yellow solid. ¹H NMR: (400 MHz, DMSO-d₆) δ: ppm 10.78 (s, 1H), 9.51 (s, 1H), 8.96-8.90 (m, 1H), 8.78 (s, 1H), 7.82 (d, J=2.0 Hz, 1H), 7.41 (s, 1H), 7.33 (d, J=2.0 Hz, 1H), 7.28 (s, 1H), 4.36-4.34 (m, 2H), 3.74-3.72 (m, 2H), 3.34 (s, 3H), 2.64 (s, 3H), 2.20 (s, 3H).

Step B: A solution of N-(1-(6-(furan-3-yl)-4-(2-methoxyethoxy)pyridin-2-yl)-3-methyl-1H-pyrazolo[4,3-c]pyridin-6-yl)acetamide (90 mg, 0.221 mmol) and Pd/C (117.5 mg, 0.11 mmol, 10% purity) in MeOH (10 mL) was stirred at 40° C. under 25 psi of H₂ atmosphere for 24 h. The mixture was filtered and the filtrate was concentrated to give the crude, which was by Prep-HPLC-F (Gradient: 35-65% MeCN) to give N-(1-(4-(2-methoxyethoxy)-6-(tetrahydrofuran-3-yl)pyridin-2-yl)-3-methyl-1H-pyrazolo[4,3-c]pyridin-6-yl)acetamide (16 mg, 17.6% yield) as a white solid. LCMS m/z=41.1 [M+H]⁺. ¹H NMR: H NMR (400 MHz, CDCl₃) δ: ppm 9.39 (s, 1H), 8.66 (s, 1H), 8.06 (s, 1H), 7.36 (d, J=2.0 Hz, 1H), 6.68 (d, J=2.0 Hz, 1H). 4.29-4.27 (m, 3H), 4.24-4.19 (m, 1H), 4.02-4.00 (m, 2H), 3.81-3.78 (m, 2H), 3.64-3.56 (m, 1H), 3.47 (s, 3H), 2.65 (s, 3H), 2.52-2.44 (m, 2H), 2.25 (s, 3H).

### Example 36 and 37: (R)—N-(3-cyclopropyl-1-(4-(2-methoxyethoxy)-6-(tetrahydrofuran-3-yl)pyridin-2-yl)-1H-pyrazolo[4,3-c]pyridin-6-yl)acetamide and (S)—N-(3-cyclopropyl-1-(4-(2-methoxyethoxy)-6-(tetrahydrofuran-3-yl)pyridin-2-yl)-1H-pyrazolo[4,3-c]pyridin-6-yl)acetamide

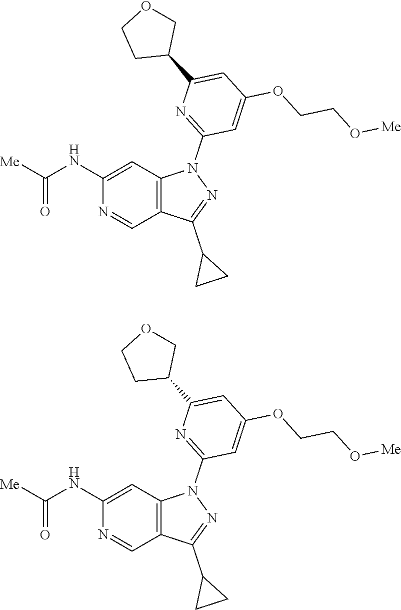 [SMILES: COCCOc1cc([C@H]2CCOC2)nc(-n2nc(C3CC3)c3cnc(NC(C)=O)cc32)c1 ; COCCOc1cc([C@@H]2CCOC2)nc(-n2nc(C3CC3)c3cnc(NC(C)=O)cc32)c1]

Step A: To a solution of N-(3-cyclopropyl-1H-pyrazolo[4,3-c]pyridin-6-yl)acetamide (Preparation 11, 300 mg, 1.39 mmol) in dioxane (10 mL) was added 2-chloro-6-(furan-3-yl)-4-(2-methoxyethoxy)pyridine (Preparation 136, 351.9 mg, 1.39 mmol), Cs₂CO₃ (904.1 mg, 2.77 mmol) and BrettPhos Pd G₃ (251.5 mg, 0.277 mmol) and the reaction was stirred at 100° C. for 16 h under N₂. The mixture was poured into H₂O (30 mL) and extracted with EtOAc (3×30 mL). The combined organic phase was washed with brine (2×20 mL), dried over Na₂SO₄ and filtered. The filtrate was concentrated under reduced pressure and the residue was purified by chromatography on silica gel (EtOAc=100%) to give N-(3-cyclopropyl-1-(6-(furan-3-yl)-4-(2-methoxyethoxy)pyridin-2-yl)-1H-pyrazolo[4,3-c]pyridin-6-yl)acetamide (288.6 mg, 48.0% yield) as a white solid. ¹H NMR: (400 MHz, CDCl₃) δ ppm: 9.59 (s, 1H), 8.72 (s, 1H), 8.60 (s, 1H), 8.12 (s, 1H), 7.55 (s, 1H), 7.36 (d, J=2.0 Hz, 1H), 7.05 (s, 1H), 6.89 (d, J=2.0 Hz, 1H), 4.32-4.30 (m, 2H), 3.83-3.81 (m, 2H), 3.49 (s, 3H), 2.32-2.25 (m, 4H), 1.22-1.14 (m, 4H).

Step B: To a solution of N-(3-cyclopropyl-1-(6-(furan-3-yl)-4-(2-methoxyethoxy)pyridin-2-yl)-1H-pyrazolo[4,3-c]pyridin-6-yl)acetamide (288.6 mg, 0.666 mmol) in MeOH (15.0 mL) was added Pd/C (141.7 mg, 0.133 mmol, 10% purity) and the reaction was stirred at 45° C. for 16 h under H₂ at 40 psi atmosphere. The reaction was filtered, concentrated and purified by chromatography on silica gel (EtOAc=100%) to give N-(3-cyclopropyl-1-(4-(2-methoxyethoxy)-6-(tetrahydrofuran-3-yl)pyridin-2-yl)-1H-pyrazolo[4,3-c]pyridin-6-yl)acetamide (52.0 mg, 17.8% yield) as a white solid. This was further purified by SFC (Condition: 0.1% NH₃·H₂O MEOH; Column: DAICEL CHIRALPAK AD (250 mm*30 mm,10 um)); Begin B: 40%; End B: 40%; Flow Rate(ml/min): 70) to give

peak 1, enantiomer 1, (21.7 mg) as a white solid. LCMS m/z=438.2 [M+H]^{+ 1}H NMR: (400 MHz, CDCl₃) δ ppm: 9.45 (s, 1H), 8.71 (s, 1H), 8.68 (s, 1H), 7.33 (s, 1H), 6.66 (s, 1H), 4.29-4.22 (m, 4H), 4.00-3.98 (m, 2H), 3.80-3.79 (m, 2H), 3.60-3.59 (m, 1H), 3.47 (s, 3H), 2.47-2.46 (m, 2H), 2.27-2.26 (m, 4H), 1.25-1.17 (m, 4H). and

peak 2, enantiomer 2 (24.9 mg, 31.1% yield) as a white solid. LCMS m/z=438.2 [M+H]^{+ 1}H NMR: (400 MHz, CDCl₃) δ ppm: 9.48 (s, 1H), 8.77 (s, 1H), 8.71 (s, 1H), 7.33 (s, 1H), 6.67 (s, 1H), 4.30-4.20 (m, 4H), 4.01-3.98 (m, 2H), 3.96-3.79 (m, 2H), 3.62-3.60 (m, 1H), 3.47 (s, 3H), 2.49-2.47 (m, 2H), 2.27-2.26 (m, 4H), 1.25-1.15 (m, 4H).

### Example 38 and 39: (R)—N-(3-ethyl-1-(4-(2-methoxyethoxy)-6-(tetrahydrofuran-3-yl)pyridin-2-yl)-1H-pyrazolo[4,3-c]pyridin-6-yl)acetamide and (S)—N-(3-ethyl-1-(4-(2-methoxyethoxy)-6-(tetrahydrofuran-3-yl)pyridin-2-yl)-1H-pyrazolo[4,3-c]pyridin-6-yl)acetamide

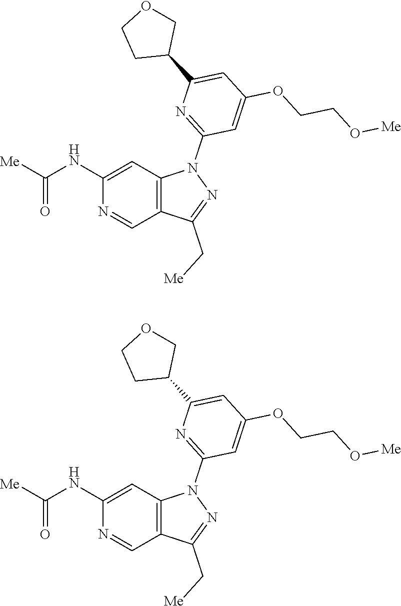 [SMILES: CCc1nn(-c2cc(OCCOC)cc([C@H]3CCOC3)n2)c2cc(NC(C)=O)ncc12 ; CCc1nn(-c2cc(OCCOC)cc([C@@H]3CCOC3)n2)c2cc(NC(C)=O)ncc12]

(R)—N-(3-ethyl-1-(4-(2-methoxyethoxy)-6-(tetrahydrofuran-3-yl)pyridin-2-yl)-1H-pyrazolo[4,3-c]pyridin-6-yl)acetamide and (S)—N-(3-ethyl-1-(4-(2-methoxyethoxy)-6-(tetrahydrofuran-3-yl)pyridin-2-yl)-1H-pyrazolo[4,3-c]pyridin-6-yl)acetamide were obtained as white solids from 2-chloro-6-(furan-3-yl)-4-(2-methoxyethoxy)pyridine (Preparation 136) and N-(3-ethyl-1H-pyrazolo[4,3-c]pyridin-6-yl)acetamide (Preparation 9), following a similar procedure to that described in Example 36 and 37.

Peak 1, enantiomer 1, (30.9 mg, 38.6% yield). LCMS m/z=426.2 [M+H]^{+ 1}H NMR: (400 MHz, CDCl₃) δ ppm: 9.39 (s, 1H), 8.69 (s, 1H), 8.20 (s, 1H), 7.38 (d, J=2.0 Hz, 1H), 6.66 (d, J=2.0 Hz, 1H), 4.29-4.27 (m, 4H), 4.02-3.97 (m, 2H), 3.81-3.79 (m, 2H), 3.78-3.60 (m, 1H), 3.47 (s, 3H), 3.04 (q, J=7.6 Hz, 2H), 2.50-2.46 (m, 2H), 2.24 (s, 3H), 1.46 (d, J=7.6 Hz, 3H).

Peak 2, Enantiomer 2, (31.2 mg, 39%). LCMS m/z=426.2 [M+H]^{+ 1}H NMR: (400 MHz, CDCl₃) δ ppm: 9.39 (s, 1H), 8.69 (s, 1H), 8.17 (s, 1H), 7.38 (s, 1H), 6.67 (s, 1H), 4.29-4.27 (m, 4H), 4.02-3.99 (m, 2H), 3.81-3.79 (m, 2H), 3.78-3.60 (m, 1H), 3.47 (s, 3H), 3.06 (q, J=7.6 Hz, 2H), 2.50-2.46 (m, 2H), 2.24 (s, 3H), 1.46 (t, J=7.6 Hz, 3H).

### Example 40 and 41: N-(3-cyclopropyl-1-(2-(1,1-difluoroethyl)-6-(oxetan-3-yl)pyridin-4-yl)-1H-pyrazolo[4,3-c]pyridin-6-yl)acetamide and N-(3-cyclopropyl-1-(6-(1,1-difluoroethyl)-4-(oxetan-3-yl)pyridin-2-yl)-1H-pyrazolo[4,3-c]pyridin-6-yl)acetamide

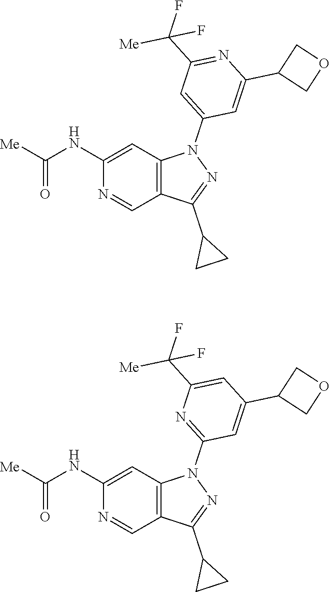 [SMILES: CC(=O)Nc1cc2c(cn1)c(C1CC1)nn2-c1cc(C2COC2)nc(C(C)(F)F)c1 ; CC(=O)Nc1cc2c(cn1)c(C1CC1)nn2-c1cc(C2COC2)cc(C(C)(F)F)n1]

Step A: To a solution of 2,4-dichloro-6-(1,1-difluoroethyl)pyridine (Preparation 127, 250 mg, 1.18 mmol) and 3-bromooxetane (161.5 mg, 1.18 mmol) in DME (4 mL) were added TTMSS (293.2 mg, 1.18 mmol), NiCl₂·glyme (25.8 mg, 0.118 mmol), dtbbpy (31.6 mg, 0.118 mmol), Ir[dF(CF₃)ppy]₂(dtbbpy)PF₆ (13.2 mg, 11.8 μmol) and LiOH (56.5 mg, 2.36 mmol). The mixture was stirred and irradiated with blue LEDs for 12 h at 20° C. under N₂. The reaction was concentrated and the residue was purified by chromatography on silica gel (PE/EtOAc=3/1) to give a mixture of 2-chloro-6-(1,1-difluoroethyl)-4-(oxetan-3-yl)pyridine and 4-chloro-2-(1,1-difluoroethyl)-6-(oxetan-3-yl)pyridine (85 mg, 30.9% yield) as a yellow oil. LCMS m/z=234.1 [M+H]⁺

Step B: To a solution of this oil (150 mg, 0.642 mmol), N-(3-cyclopropyl-1H-pyrazolo[4,3-c]pyridin-6-yl)acetamide (Preparation 11, 138.8 mg, 0.642 mmol) in dioxane (5 mL) was added BrettPhos Pd G3 (116.4 mg, 0.128 mmol) and Cs₂CO₃ (418.4 mg, 1.28 mmol). The reaction mixture was stirred at 100° C. for 12 h and then was concentrated in vacuo. The crude material was purified by Prep-HPLC-C (Gradient: 37-57% MeCN) to give N-(3-cyclopropyl-1-(2-(1,1-difluoroethyl)-6-(oxetan-3-yl)pyridin-4-yl)-1H-pyrazolo[4,3-c]pyridin-6-yl)acetamide (37.1 mg, 14% yield) as a white solid. LCMS m/z=414.1 [M+H]. ¹H NMR: (400 MHz, CDCl₃) δ: ppm 8.76 (s, 2H), 8.23 (s, 1H), 7.98 (d, J=2.0 Hz, 1H), 7.77 (s, 1H), 5.10-5.14 (m, 2H), 5.04-5.07 (m, 2H), 4.49-4.56 (m, 1H), 2.27-2.31 (m, 4H), 2.11 (t, J=18.4 Hz, 3H), 1.17-1.24 (m, 4H) and N-(3-cyclopropyl-1-(6-(1,1-difluoroethyl)-4-(oxetan-3-yloxy)pyridin-2-yl)-1H-pyrazolo[4,3-c]pyridin-6-yl)acetamide (8.6 mg, 3.2% yield) as a white solid. LCMS m/z=414.1 [M+H]⁺. 1H NMR: (500 MHz, CDCl₃) δ: ppm 9.42 (br s, 1H), 8.74 (s, 1H), 8.27 (s, 1H), 8.01 (s, 1H), 7.57 (s, 1H), 5.16-5.13 (m, 2H), 4.86-4.84 (m, 2H), 4.40-4.34 (m, 1H), 2.36-2.28 (m, 4H), 2.27 (s, 3H), 1.25-1.23 (m, 2H), 1.20-1.18 (m, 2H).

### Example 42: N-(1-(5-(3-methoxytetrahydrofuran-3-yl)pyridin-3-yl)-1H-pyrazolo[4,3-c]pyridin-6-yl)acetamide

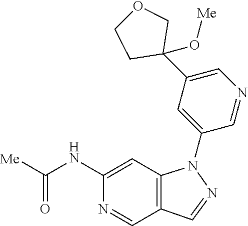 [SMILES: COC1(c2cncc(-n3ncc4cnc(NC(C)=O)cc43)c2)CCOC1]

To a mixture of 3-bromo-5-(3-methoxytetrahydrofuran-3-yl)pyridine (Preparation 50, 100 mg, 0.387 mmol) in DMSO (3 mL) was added N-(1H-pyrazolo[4,3-c]pyridin-6-yl)acetamide (Preparation 2, 68.3 mg, 0.387 mmol), CuI (14.8 mg, 0.077 mmol), 2-(dimethylamino)acetic acid (15.9 mg, 0.155 mmol), K₃PO₄ (246.7 mg, 1.16 mmol) and the mixture was stirred at 120° C. for 12 h under N₂. The mixture was filtered and purified by Prep-HPLC-K (Gradient: 15-45% MeCN) to give N-(1-(5-(3-methoxytetrahydrofuran-3-yl)pyridin-3-yl)-1H-pyrazolo[4,3-c]pyridin-6-yl)acetamide (8.3 mg, 6% yield) as a white solid. LCMS m/z=354.2 [M+H]⁺ 1H NMR: (500 MHz, MeOH-d₄) δ ppm: 8.98 (s, 1H), 8.92 (s, 1H), 8.67 (s, 1H), 8.61 (s, 1H), 8.44 (s, 1H), 8.26 (d, J=2.5 Hz, 1H), 4.28-4.26 (m, 1H), 4.14-4.11 (m, 2H), 3.91-3.89 (m, 1H), 3.24 (s, 3H), 2.66-2.63 (m, 1H), 2.49-2.43 (m, 1H), 2.21 (s, 3H).

### Example 43: N-(1-(6-(1-methoxy-2,2-dimethylpropyl)pyridin-2-yl)-1H-pyrazolo[4,3-c]pyridin-6-yl)acetamide

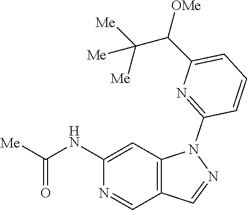 [SMILES: COC(c1cccc(-n2ncc3cnc(NC(C)=O)cc32)n1)C(C)(C)C]

To a solution of N-(1H-pyrazolo[4,3-c]pyridin-6-yl)acetamide (Preparation 2, 100 mg, 0.568 mmol) in DMSO (3 mL) was added 2-bromo-6-(1-methoxy-2,2-dimethylpropyl)pyridine (Preparation 49, 146.5 mg, 0.568 mmol), K₂CO₃ (235.3 mg, 1.7 mmol), L-proline (26.1 mg, 0.227 mmol) and CuI (21.6 mg, 0.114 mmol) and the reaction was stirred at 90° C. for 2 h. The cooled mixture was concentrated to give the crude, which was purified by Prep-HPLC-F (Gradient: 51-81% MeCN) to give N-(1-(6-(1-methoxy-2,2-dimethylpropyl)pyridin-2-yl)-1H-pyrazolo[4,3-c]pyridin-6-yl)acetamide (16.6 mg, 8.3% yield) as a yellow solid. LCMS m/z=354.2 [M+H]⁺ 1H NMR: (500 MHz, CDCl₃) δ ppm: 9.51 (br s, 1H), 8.79 (s, 1H), 8.25-8.22 (m, 2H), 7.91-7.83 (m, 2H), 7.30 (d, J=6.5 Hz, 1H), 4.19 (s, 1H), 3.31 (s, 3H), 2.27 (s, 3H), 1.04 (s, 9H).

### Example 44: N-(1-(6-(1-methoxycyclopentyl)pyridin-2-yl)-1H-pyrazolo[4,3-c]pyridin-6-yl)acetamide

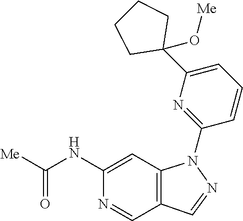 [SMILES: COC1(c2cccc(-n3ncc4cnc(NC(C)=O)cc43)n2)CCCC1]

To a solution of N-(1H-pyrazolo[4,3-c]pyridin-6-yl)acetamide (Preparation 2, 100 mg, 0.57 mmol) in dioxane (5 mL) was added 2-bromo-6-(1-methoxycyclopentyl)pyridine (Preparation 51, 145.4 mg, 0.57 mmol), K₂CO₃ (235.4 mg, 1.70 mmol), CuI (43.2 mg, 0.23 mmol) and N¹,N²-dimethylethane-1,2-diamine (10.0 mg, 0.114 mmol) and the reaction stirred at 90° C. for 2 h. The cooled mixture was concentrated in vacuo and the crude was purified by Prep-HPLC-C (Gradient: 39-69% MeCN) to give N-(1-(6-(1-methoxycyclopentyl)pyridin-2-yl)-1H-pyrazolo[4,3-c]pyridin-6-yl)acetamide (12.8 mg, 6.4% yield) as a white solid. LCMS m/z=352.2 [M+H]⁺. ¹H NMR: (400 MHz, CDCl₃) δ ppm: 9.36 (br s, 1H), 8.78 (s, 1H), 8.22 (s, 1H), 8.13 (s, 1H), 7.87-7.83 (m, 2H), 7.46 (d, J=6.0 Hz, 1H), 3.20 (s, 3H), 2.35-2.34 (m, 2H), 2.26 (s, 3H), 2.23-2.22 (m, 2H), 1.93-1.92 (m, 4H).

Examples 45 to 81: The compounds in the following table were prepared from the appropriate pyridine and N-(pyrazolo[4,3-c]pyridin-6-yl)acetamide, following a similar procedure to that described in Example 44.

| | |
|---|---|
| Example No | Name, Structure, Pyridine starting material, HPLC, Data |

Acetamide: N-(1H-pyrazolo[4,3-c]pyridin-6-yl)acetamide (Preparation 2)

| | |
|---|---|
| 45 | N-(1-(6-(3-methyltetrahydrofuran-3-yl)pyridin-2-yl)-1H-pyrazolo[4,3- |
| | c]pyridin-6-yl)acetamide |
| | 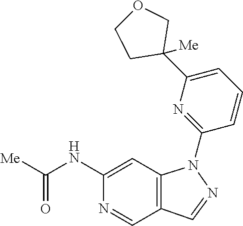 [SMILES: CC(=O)Nc1cc2c(cn1)cnn2-c1cccc(C2(C)CCOC2)n1] |
| | Pyridine: 2-bromo-6-(3-methyltetrahydrofuran-3-yl)pyridine (Preparation |
| | 75), Prep-HPLC-F (Gradient: 28-58% MeCN) white solid, 20.2 mg, 72.5%. |
| | LCMS m/z = 338.2 [M + H]⁺. 1H NMR (500 MHz, CDCl₃) δ ppm: 9.44 (s, |
| | 1H), 8.78 (s, 1H), 8.21 (s, 1H), 8.16 (br s, 1H), 7.86 (d, J = 8.5 Hz,, 1H), |
| | 7.81 (t, J = 7.5 Hz, 1H), 7.19 (d, J = 7.0 Hz, 1H), 4.24-4.23 (m, 1H), 4.12- |
| | 4.06 (m, 3H), 2.69-2.65 (m, 1H), 2.27-2.22 (m, 4H), 1.64 (s, 3H). |
| 46 | N-(1-(6-(1,2-dimethoxypropan-2-yl)pyridin-2-yl)-1H-pyrazolo[4,3- |
| | c]pyridin-6-yl)acetamide |
| | 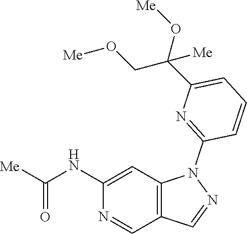 [SMILES: COCC(C)(OC)c1cccc(-n2ncc3cnc(NC(C)=O)cc32)n1] |
| | Pyridine: 2-bromo-6-(1,2-dimethoxypropan-2-yl)pyridine (Preparation 53) |
| | Prep-HPLC-C (Gradient: 27-57% MeCN) |
| | white solid (63.8 mg, 46.7% yield). LCMS m/z = 356.2 [M + H]⁺ |
| | 1H NMR (400 MHz, DMSO-d₆) δ ppm: 10.69 (s, 1H), 9.35 (s, 1H), 8.95 |
| | (s, 1H), 8.53 (s, 1H), 8.10 (t, J = 8.0 Hz, 1H), 7.87 (d, J = 8.4 Hz, 1H), 7.47 |
| | (d, J = 7.6 Hz, 1H), 3.83 (s, 2H), 3.22 (s, 6H), 2.16 (s, 3H), 1.62 (s, 3H). |
| 47 | N-(1-(6-(3-cyanotetrahydrofuran-3-yl)pyridin-2-yl)-1H-pyrazolo[4,3- |
| | c]pyridin-6-yl)acetamide |
| | 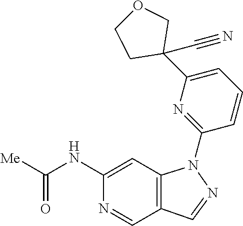 [SMILES: CC(=O)Nc1cc2c(cn1)cnn2-c1cccc(C2(C#N)CCOC2)n1] |
| | Pyridine: 3-(6-bromopyridin-2-yl)tetrahydrofuran-3-carbonitrile |
| | (Preparation 87), |
| | Prep-HPLC-C (Gradient: 28-58% MeCN) |
| | 35.9 mg, 25.4% as a yellow solid. LCMS m/z = 349.2 [M + H]⁺. ¹H NMR: |
| | (400 MHz, CDCl₃) δ ppm: 9.34 (s, 1H), 8.80 (s, 1H), 8.24 (s, 1H), 8.13 (s, |
| | 1H), 8.03 (d, J = 5.6 Hz, 1H), 7.94-7.90 (m 1H), 7.64 (d, J = 7.2 Hz, 1H), |
| | 4.54-4.49 (m, 2H), 4.35-4.29 (m, 2H), 3.29-3.25 (m, 1H), 2.86-2.80 (m, |
| | 1H), 2.26 (s, 3H). |
| 48 | N-(1-(6-(3-difluoromethoxy)tetrahydrofuran-3-yl)pyridin-2-yl)-1H- |
| | pyrazolo[4,3-c]pyridin-6-yl)acetamide |
| | 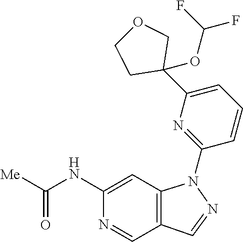 [SMILES: CC(=O)Nc1cc2c(cn1)cnn2-c1cccc(C2(OC(F)F)CCOC2)n1] |
| | Pyridine: 2-bromo-6-(3-(difluoromethoxy)tetrahydrofuran-3-yl)pyridine |
| | (Preparation 62), |
| | Prep-HPLC-F (Gradient: 32-62% MeCN) 44.2 mg, 51.4% as a white solid. |
| | LCMS m/z = 390.2 [M + H]⁺. 1H NMR: (400 MHz, CDCl₃) δ ppm: 9.35 (s, |
| | 1H), 8.80 (br s, 1H), 8.24 (s, 1H), 8.15 (br s, 1H), 8.02 (d, J = 8.0 Hz, 1H), |
| | 7.92 (t, J = 8.0 Hz, 1H), 7.53 (d, J = 7.6 Hz, 1H), 6.53 (t, J = 74.8 Hz, 1H), |
| | 4.46-4.22 (m, 4H), 3.23-3.15 (m, 1H), 2.75-2.70 (m, 1H), 2.26 (s, 3H). |
| 49 | N-(1-(6-(1-methoxyethyl)pyridin-2-yl)-1H-pyrazolo[4,3-c]pyridin-6- |
| | yl)acetamide |
| | 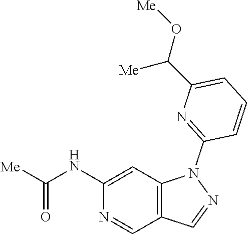 [SMILES: COC(C)c1cccc(-n2ncc3cnc(NC(C)=O)cc32)n1] |
| | Pyridine: 2-bromo-6-(1-methoxyethyl)pyridine |
| | Prep-HPLC-C (Gradient: 26-56% MeCN) |
| | 26 mg, 18% as a white solid. LCMS m/z = 312.1 [M + H]⁺ 1H NMR: (400 |
| | MHz, CDCl₃) δ ppm: 9.52 (s, 1H), 8.79 (s, 1H), 8.23 (s, 1H), 8.12 (s, 1H), |
| | 7.89-7.88 (m, 2H), 7.39-7.35 (m, 1H), 4.63 (q, J = 6.4 Hz, 1H), 3.45 (s, 3H), |
| | 2.29 (s, 3H), 1.69 (d, J = 6.8 Hz, 3H). |
| 50 | N-(1-(6-(2-methoxypropan-2-yl)pyridin-2-yl)-1H-pyrazolo[4,3-c]pyridin- |
| | 6-yl)acetamide |
| | 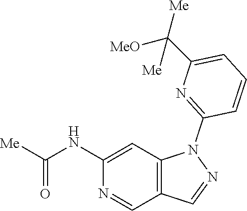 [SMILES: COC(C)(C)c1cccc(-n2ncc3cnc(NC(C)=O)cc32)n1] |
| | Pyridine: 2-bromo-6-(2-methoxypropan-2-yl)pyridine (Preparation 131) |
| | Prep-HPLC-C (Gradient: 30-60% MeCN) |
| | 33.8 mg, 15.3% yield as white solid. LCMS m/z = 326.2 [M + H]⁺ |
| | 1H NMR: (400 MHz, DMSO-d₆) δ ppm: 10.67 (s, 1H), 9.37 (s, 1H), 8.95 |
| | (s, 1H), 8.53 (s, 1H), 8.02 (t, J = 7.6 Hz, 1H), 7.85 (d, J = 7.6 Hz, 1H), 7.46 |
| | (d, J = 8.0 Hz, 1H), 3.18 (s, 3H), 2.15 (s, 3H), 1.64 (s, 6H). |
| 51 | N-(1-(6-(3-methoxyoxetan-3-yl)pyridin-2-yl)-1H-pyrazolo[4,3-c]pyridin- |
| | 6-yl)acetamide |
| | 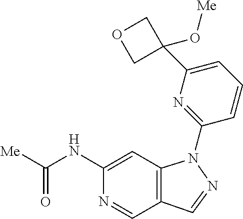 [SMILES: COC1(c2cccc(-n3ncc4cnc(NC(C)=O)cc43)n2)COC1] |
| | Pyridine: 2-bromo-6-(3-methoxyoxetan-3-yl)pyridine (Preparation 56) |
| | Prep-HPLC-C (Gradient: 23-53% MeCN) |
| | 52.4 mg, 37.7% yield as white solid. LCMS m/z = 340.2 [M + H]⁺. ¹H |
| | NMR: (500 MHz, CDCl₃) δ ppm: 9.45 (s, 1H), 8.79 (s, 1H), 8.24 (s, 1H), |
| | 8.08 (s, 1H), 8.01 (d, J = 8.5 Hz, 1H), 7.94-7.90 (m, 1H), 7.35 (d, J = 7.5 Hz, |
| | 1H), 5.30-5.28 (m, 2H), 5.05-5.03 (m, 2H), 3.33 (s, 3H), 2.26 (s, 3H). |
| 52 | N-(1-(6-(1-methoxycyclobutyl)pyridin-2-yl)-1H-pyrazolo[4,3-c]pyridin-6- |
| | yl)acetamide |
| | 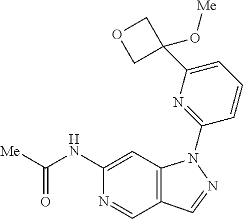 [SMILES: COC1(c2cccc(-n3ncc4cnc(NC(C)=O)cc43)n2)COC1] |
| | Pyridine: 2-bromo-6-(1-methoxycyclobutyl)pyridine (Preparation 52) |
| | Prep-HPLC-C (Gradient: 32-62% MeCN) |
| | 19.3 mg, 10.1% yield as a white solid. LCMS m/z = 338.2 [M + H]⁺ 1H |
| | NMR: (400 MHz, CDCl₃) δ ppm: 9.42 (br s, 1H), 8.80 (s, 1H), 8.24 (s, |
| | 1H), 8.14 (s, 1H), 7.94-7.86 (m, 2H), 7.41 (d, J = 7.2 Hz, 1H), 3.16 (s, 3H), |
| | 2.83-2.79 (m, 2H), 2.50-2.47 (m, 2H), 2.26 (s, 3H), 2.04-2.00 (m, 2H). |
| 53 | N-(1-(6-(1-methoxycyclopropyl)pyridin-2-yl)-1H-pyrazolo[4,3-c]pyridin- |
| | 6-yl)acetamide |
| | 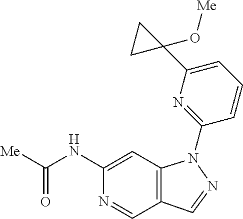 [SMILES: COC1(c2cccc(-n3ncc4cnc(NC(C)=O)cc43)n2)CC1] |
| | Pyridine: 2-bromo-6-(1-methoxycyclopropyl)pyridine (Preparation 57) |
| | Prep-HPLC-C (Gradient: 34-64% MeCN) |
| | 28.1 mg, 24.8% yield as a white solid. LCMS m/z = 324.2 [M + H]⁺ |
| | 1H NMR: (400 MHz, CDCl₃) δ ppm: 9.16 (s, 1H), 8.77 (s, 1H), 8.20 (s, |
| | 1H), 8.09 (br s, 1H), 7.85-7.80 (m, 2H), 7.56-7.51 (m, 1H), 3.46 (s, 3H), |
| | 2.28 (s, 3H), 1.77-1.74 (m, 2H), 1.45-1.41 (m, 2H). |
| 54 | N-(1-(6-(1-aminocyclopropyl)pyridin-2-yl)-1H-pyrazolo[4,3-c]pyridin-6- |
| | yl)acetamide |
| | 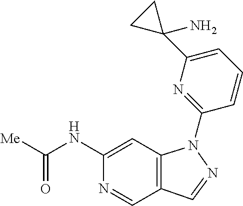 [SMILES: CC(=O)Nc1cc2c(cn1)cnn2-c1cccc(C2(N)CC2)n1] |
| | Pyridine: 1-(6-bromopyridin-2-yl)cyclopropan-1-amine |
| | Prep-HPLC-F (Gradient: 21-51% MeCN) |
| | 16.3 mg, 14.1% yield as a yellow solid. LCMS m/z = 309.2 [M + H]⁺ |
| | 1H NMR: (500 MHz, MeOH-d₄) δ ppm: 9.34 (s, 1H), 8.87 (s, 1H), 8.43- |
| | 8.33 (m, 1H), 7.92-7.82 (m, 2H), 7.45-7.34 (m, 1H), 2.25 (s, 3H), 1.61- |
| | 1.51 (m, 2H), 1.30-1.22 (m, 2H) |
| 55 | N-(1-(6-(3-fluorooxetan-3-yl)pyridin-2-yl)-1H-pyrazolo[4,3-c]pyridin-6- |
| | yl)acetamide |
| | 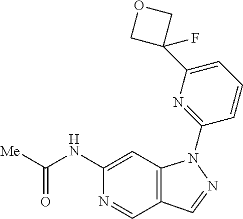 [SMILES: CC(=O)Nc1cc2c(cn1)cnn2-c1cccc(C2(F)COC2)n1] |
| | Pyridine: 2-bromo-6-(3-fluorooxetan-3-yl)pyridine (Preparation 72) |
| | Prep-HPLC-C (Gradient: 25-55% MeCN) |
| | 12.2 mg, 8.2% yield as a white solid. LCMS m/z = 328.1 [M + H]⁺ |
| | 1H NMR: (400 MHz, CDCl₃) δ ppm: 9.79 (brs, 1H), 8.54 (s, 1H), 8.26 (s, |
| | 1H), 8.01 (d, J = 8.4 Hz, 1H), 7.95-7.91 (m, 1H), 7.44 (d, J = 7.6 Hz, 1H), |
| | 5.47-5.39 (m, 2H), 5.17-5.10 (m, 2H), 2.31 (s, 3H). |
| 56 | N-(1-(6-(3-fluorotetrahydrofuran-3-yl)-4-methylpyridin-2-yl)-1H- |
| | pyrazolo[4,3-c]pyridin-6-yl)acetamide |
| | 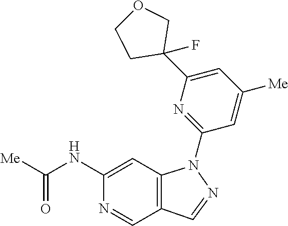 [SMILES: CC(=O)Nc1cc2c(cn1)cnn2-c1cc(C)cc(C2(F)CCOC2)n1] |
| | Pyridine: 2-bromo-6-(3-fluorotetrahydrofuran-3-yl)-4-methylpyridine |
| | (Preparation 64) |
| | Prep-HPLC-F (Gradient: 42-72% MeCN) |
| | 19.2 mg, 7.0% yield as a white solid. LCMS m/z = 356.1 [M + H]⁺ |
| | ¹H NMR: (500 MHz, CDCl₃) δ: ppm 9.36 (s, 1H), 8.78 (s, 1H), 8.22 (s, |
| | 1H), 8.10 (br s, 1H), 7.80 (s, 1H), 7.39 (s, 1H), 4.50-4.46 (m, 1H), 4.34- |
| | 4.24 (m, 3H), 3.19-3.09 (m, 1H), 2.58-2.52 (m, 1H), 2.50 (s, 3H), 2.25 (s, |
| | 3H). |
| 57 | N-(1-(6-(3-fluorooxetan-3-yl)pyridin-2-yl)-3-methyl-1H-pyrazolo[4,3- |
| | c]pyridin-6-yl)acetamide |
| | 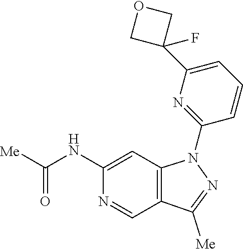 [SMILES: CC(=O)Nc1cc2c(cn1)c(C)nn2-c1cccc(C2(F)COC2)n1] |
| | Pyridine: 2-bromo-6-(3-fluorooxetan-3-yl)pyridine (Preparation 72) |
| | Prep-HPLC-C (Gradient: 29-59% MeCN) |
| | 40.1 mg, 27.9% yield as a white solid. LCMS m/z = 342.1 [M + H]⁺ |
| | ¹H NMR: (400 MHz, CDCl₃) δ ppm: 9.59 (brs, 1H), 8.79 (s, 1H), 7.97 (d, |
| | J = 8.0 Hz, 1H), 7.92-7.88 (m, 1H), 7.40 (d, J = 7.6 Hz, 1H), 5.46-5.38 (m, |
| | 2H), 5.16-5.09 (m, 2H), 2.68 (s, 3H), 2.30 (s, 3H). |
| 58 | N-(1-(6-(3-fluorotetrahydrofuran-3-yl)-4-isopropoxypyridin-2-yl)-1H- |
| | pyrazolo[4,3-c]pyridin-6-yl)acetamide |
| | 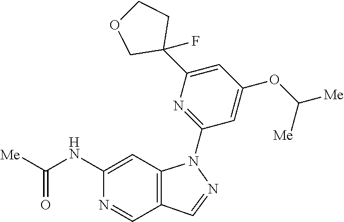 [SMILES: CC(=O)Nc1cc2c(cn1)cnn2-c1cc(OC(C)C)cc(C2(F)CCOC2)n1] |
| | Pyridine: 2-bromo-6-(3-fluorotetrahydrofuran-3-yl)-4-isopropoxypyridine |
| | (Preparation 66) |
| | Prep-HPLC-F (Gradient: 55-85% MeCN) |
| | 45.2 mg, 34.4% yield as a white solid. LCMS m/z = 400.1 [M + H]⁺ |
| | 1H NMR: (400 MHz, CDCl₃) δ: ppm 9.36 (s, 1H), 8.77 (s, 1H), 8.20 (s, |
| | 1H), 8.08 (s, 1H), 7.44 (d, J = 1.6 Hz, 1H), 7.08-7.06 (m, 1H), 4.84-4.79 (m, |
| | 1H), 4.48-4.46 (m, 1H), 4.32-4.31 (m, 2H), 4.29-4.22 (m, 1H), 3.23-3.08 |
| | (m, 1H), 2.54-2.48 (m, 1H), 2.25 (s, 3H), 1.43 (d, J = 5.6 Hz, 6H). |

Acetamide: N-(3-methyl-1H-pyrazolo[4,3-c]pyridin-6-yl)acetamide (Preparation 6)

| | |
|---|---|
| 59 | N-(1-(6-(3-fluorotetrahydrofuran-3-yl)-4-methylpyridin-2-yl)-3-methyl- |
| | 1H-pyrazolo[4,3-c]pyridin-6-yl)acetamide |
| | 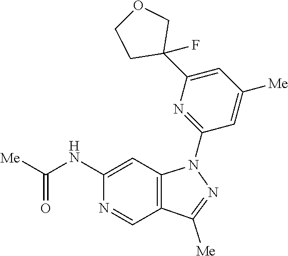 [SMILES: CC(=O)Nc1cc2c(cn1)c(C)nn2-c1cc(C)cc(C2(F)CCOC2)n1] |
| | Pyridine: 2-bromo-6-(3-fluorotetrahydrofuran-3-yl)-4-methylpyridine |
| | (Preparation 64) |
| | Prep-HPLC-F (Gradient: 50-65% MeCN) |
| | 27.6 mg, 19.4% yield as a white solid. LCMS m/z = 370.1 [M + H]⁺ |
| | ¹H NMR: (400 MHz, CDCl₃) δ: ppm 9.30 (s, 1H), 8.68 (s, 1H), 8.09 (br s, |
| | 1H), 7.75 (s, 1H), 7.34 (s, 1H), 4.50-4.47 (m, 1H), 4.34-4.22 (m, 3H), |
| | 3.21-3.08 (m, 1H), 2.66 (s, 3H), 2.56-2.50 (m, 1H), 2.48 (s, 3H), 2.24 (s, |
| | 3H). |
| 60 | N-(1-(6-(3-methoxytetrahydrofuran-3-yl)-4-methylpyridin-2-yl)-3-methyl- |
| | 1H-pyrazolo[4,3-c]pyridin-6-yl)acetamide |
| | 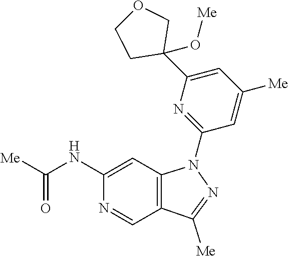 [SMILES: COC1(c2cc(C)cc(-n3nc(C)c4cnc(NC(C)=O)cc43)n2)CCOC1] |
| | Pyridine: 2-bromo-6-(3-methoxytetrahydrofuran-3-yl)-4-methylpyridine |
| | (Preparation 124) |
| | Prep-HPLC-C (Gradient: 33-63% MeCN) |
| | 18 mg, 28.5% yield. LCMS m/z = 382.2 [M + H]⁺ |
| | 1H NMR: (500 MHz, CDCl₃) δ ppm: 9.28 (s, 1H), 8.68 (s, 1H), 8.07 (s, |
| | 1H), 7.74 (s, 1H), 7.23 (s, 1H), 4.31-4.21 (m, 3H), 4.11-4.08 (m, 1H), 3.23 |
| | (s, 3H), 2.96-2.91 (m, 1H), 2.67 (s, 3H), 2.60-2.57 (m, 1H), 2.47 (s, 3H), |
| | 2.25 (s, 3H). |
| 61 | N-(3-methyl-1-(6-(oxetan-3-yl)pyridin-2-yl)-1H-pyrazolo[4,3-c]pyridin-6- |
| | yl)acetamide |
| | 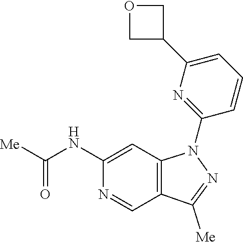 [SMILES: CC(=O)Nc1cc2c(cn1)c(C)nn2-c1cccc(C2COC2)n1] |
| | Pyridine: 2-bromo-6-(oxetan-3-yl)pyridine (Preparation 135) |
| | Prep-HPLC-F (Gradient: 36-65% MeCN) |
| | 76.1 mg, 28.0% yield as a white solid. LCMS m/z = 324.1 [M + H]⁺ |
| | ¹H NMR: (400 MHz, CDCl₃) δ: ppm 9.44 (s, 1H), 8.66 (s, 1H), 8.28 (br s, |
| | 1H), 7.86-7.77 (m, 2H), 7.10 (d, J = 7.2 Hz, 1H), 5.18-5.16 (m, 4H), 4.57- |
| | 4.49 (m, 1H), 2.66 (s, 3H), 2.26 (s, 3H). |

Acetamide: N-(3-cyclopropyl-1H-pyrazolo[4,3-c]pyridin-6-yl)acetamide (Preparation 11)

| | |
|---|---|
| 62 | N-(3-cyclopropyl-1-(6-(3-fluorooxetan-3-yl)pyridin-2-yl)-1H- |
| | pyrazolo[4,3-c]pyridin-6-yl)acetamide |
| | 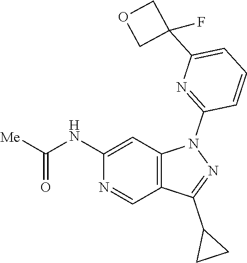 [SMILES: CC(=O)Nc1cc2c(cn1)c(C1CC1)nn2-c1cccc(C2(F)COC2)n1] |
| | Pyridine: 2-bromo-6-(3-fluorooxetan-3-yl)pyridine (Preparation 72) |
| | Prep-HPLC-C (Gradient: 38 to 68% MeCN) |
| | 21.9 mg, 27.7% yield as a white solid. LCMS m/z = 368.1 [M + H]⁺ |
| | ¹H NMR: (500 MHz, CDCl₃) δ ppm: 9.41 (s, 1H), 8.77 (s, 1H), 8.15 (s, |
| | 1H), 7.95 (d, J = 9.0 Hz, 1H), 7.90-7.86 (m, 1H), 7.37 (d, J = 8.0 Hz, 1H), |
| | 5.48-5.46 (m, 2H), 5.16-5.10 (m, 2H), 2.33-2.29 (m, 1H), 2.27 (s, 3H), |
| | 1.25-1.24 (m, 2H), 1.20-1.18 (m, 2H). |
| 63 | N-(3-cyclopropyl-1-(6-(3-fluorotetrahydrofuran-3-yl)-4-methoxypyridin-2- |
| | yl)-1H-pyrazolo[4,3-c]pyridin-6-yl)acetamide |
| | 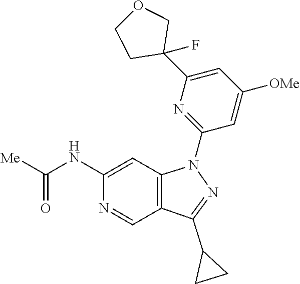 [SMILES: COc1cc(-n2nc(C3CC3)c3cnc(NC(C)=O)cc32)nc(C2(F)CCOC2)c1] |
| | Pyridine: 2-bromo-6-(3-fluorotetrahydrofuran-3-yl)-4-methoxypyridine |
| | (Preparation 65) |
| | Purified by chromatography on silica gel (PE/EtOAc = 1/1) |
| | 13.8 g, 21.8% yield as a yellow solid. LCMS m/z = 412.1 [M + H]⁺ |
| | ¹H NMR: (500 MHz, CDCl₃) δ: ppm 9.29 (s, 1H), 8.72 (s, 1H), 8.07 (br s, |
| | 1H), 7.38 (d, J = 2.0 Hz, 1H), 7.06-7.04 (m, 1H), 4.48-4.31 (m, 1H), 4.29- |
| | 4.21 (m, 3H), 3.97 (s, 3H), 3.16-3.09 (m, 1H), 2.55-2.50 (m, 1H), 2.30- |
| | 2.27 (m 1H), 2.24 (s, 3H), 1.23-1.14 (m, 4H). |
| 64 | N-(3-cyclopropyl-1-(6-(3-fluorooxetan-3-yl)-4-methylpyridin-2-yl)-1H- |
| | pyrazolo[4,3-c]pyridin-6-yl)acetamide |
| | 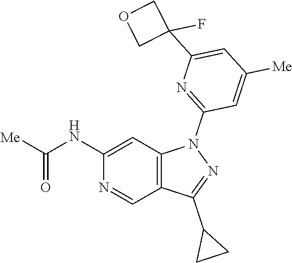 [SMILES: CC(=O)Nc1cc2c(cn1)c(C1CC1)nn2-c1cc(C)cc(C2(F)COC2)n1] |
| | Pyridine: 2-bromo-6-(3-fluorooxetan-3-yl)-4-methylpyridine (Preparation |
| | 67) |
| | Purified by chromatography on silica gel (PE/EtOAc = 1/2) |
| | 13.7 mg, 11.1% yield as a white solid. LCMS m/z = 382.1 [M + H]⁺ |
| | ¹H NMR: (400 MHz, CDCl₃) δ: ppm 9.75 (br s, 1H), 9.60 (s, 1H), 8.75 (s, |
| | 1H), 7.77 (s, 1H), 5.38 (d, J = 7.2 Hz, 1H), 5.33 (d, J = 7.6 Hz, 1H), 5.13 (d, |
| | J = 7.2 Hz, 1H), 5.07 (d, J = 7.2 Hz, 1H), 2.50 (s, 3H), 2.31 (s, 3H), 2.28-2.26 |
| | (m, 1H), 1.27-1.24 (m, 4H). |
| 65 | N-(3-cyclopropyl-1-(6-(3-fluorooxetan-3-yl)-4-methoxypyridin-2-yl)-1H- |
| | pyrazolo[4,3-c]pyridin-6-yl)acetamide |
| | 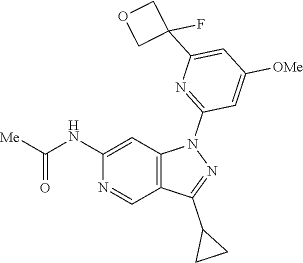 [SMILES: COc1cc(-n2nc(C3CC3)c3cnc(NC(C)=O)cc32)nc(C2(F)COC2)c1] |
| | Pyridine: 2-bromo-6-(3-fluorooxetan-3-yl)-4-methoxypyridine |
| | (Preparation 68) |
| | Prep-HPLC-C (Gradient: 40-70% MeCN) |
| | 15.4 mg, yield: 10.2% as a yellow solid. LCMS m/z = 398.1 [M + H]⁺ |
| | ¹H NMR (400 MHz, CDCl₃) δ ppm: 9.39 (s, 1H), 8.74 (s, 1H), 8.03 (s, |
| | 1H), 7.42 (d, J = 2.0 Hz, 1H), 6.91 (d, J = 1.6 Hz, 1H), 5.45-5.36 (m, 2H), |
| | 5.11-5.03 (m, 2H), 3.96 (s, 3H), 2.32-2.29 (m, 1H), 2.25 (s, 3H), 1.23-1.15 |
| | (m, 4H) |
| 66 | N-(3-cyclopropyl-1-(6-(3-fluorooxetan-3-yl)-4-isopropoxypyridin-2-yl)- |
| | 1H-pyrazolo[4,3-c]pyridin-6-yl)acetamide |
| | 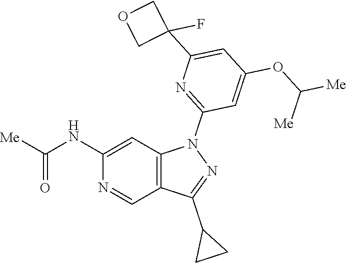 [SMILES: CC(=O)Nc1cc2c(cn1)c(C1CC1)nn2-c1cc(OC(C)C)cc(C2(F)COC2)n1] |
| | Pyridine: 2-bromo-6-(3-fluorooxetan-3-yl)-4-isopropoxypyridine |
| | (Preparation 69) |
| | Prep-HPLC-C (Gradient: 50-80% MeCN) |
| | 32.6 mg, 22.2% yield as a white solid. LCMS m/z = 426.2 [M + H]⁺ |
| | ¹H NMR: (500 MHz, CDCl₃) δ: ppm 9.38 (s, 1H), 8.72 (s, 1H), 8.25 (br s, |
| | 1H), 7.35 (s, 1H), 6.87 (s, 1H), 5.45-5.38 (m, 2H), 5.10-5.04 (m, 2H), |
| | 4.81-4.77 (m, 1H), 2.31-2.27 (m, 1H), 2.25 (s, 3H), 1.41 (d, J = 6.0 Hz, 6H), |
| | 1.22-1.15 (m, 4H). |
| 67 | N-(3-cyclopropyl-1-(6-(3-fluorooxetan-3-yl)-4-(2-methoxyethoxy)pyridin- |
| | 2-yl)-1H-pyrazolo[4,3-c]pyridin-6-yl)acetamide |
| | 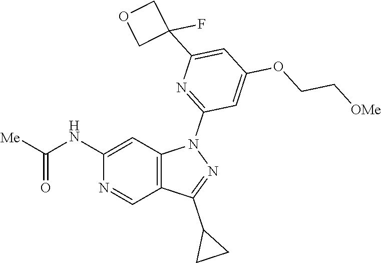 [SMILES: COCCOc1cc(-n2nc(C3CC3)c3cnc(NC(C)=O)cc32)nc(C2(F)COC2)c1] |
| | Pyridine: 2-bromo-6-(3-fluorooxetan-3-yl)-4-(2-methoxyethoxy)pyridine |
| | (Preparation 70) |
| | Prep-HPLC-C (Gradient: 34.64% MeCN) |
| | 52.1 mg, 42.5% yield as a white solid. LCMS m/z = 442.1 [M + H]⁺ |
| | ¹H NMR: (500 MHz, DMSO-d₆) δ ppm: 10.70 (s, 1H), 9.25 (s, 1H), 8.97 |
| | (s, 1H), 7.38 (s, 1H), 7.03 (d, J = 1.5 Hz, 1H), 5.33-5.25 (m, 2H), 4.97-4.90 |
| | (m, 2H), 4.34-4.33 (m, 2H), 3.71-3.70 (m, 2H), 3.32 (s, 3H), 2.48-2.46 (m, |
| | 1H), 2.14 (s, 3H), 1.20-1.14 (m, 4H). |
| 68 | N-(3-cyclopropyl-1-(6-(2-fluoro-1-methoxypropan-2-yl)pyridin-2-yl)-1H- |
| | pyrazolo[4,3-c]pyridin-6-yl)acetamide |
| | 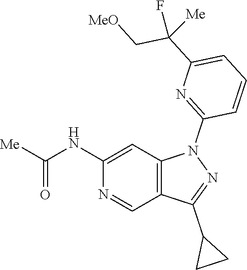 [SMILES: COCC(C)(F)c1cccc(-n2nc(C3CC3)c3cnc(NC(C)=O)cc32)n1] |
| | Pyridine: 2-bromo-6-(2-fluoro-1-methoxypropan-2-yl)pyridine |
| | (Preparation 71) |
| | Prep-HPLC-C (Gradient: 44-74% MeCN) |
| | 37.6 mg, 35.3% yield as a white solid. LCMS m/z = 384.2 [M + H]⁺ |
| | ¹H NMR: (500 MHz, CDCl₃) δ ppm: 9.34 (s, 1H), 8.71 (s, 1H), 8.08 (s, |
| | 1H), 7.83-7.80 (m, 2H), 7.44-7.41 (m, 1H), 4.18-4.10 (m, 1H), 4.01-3.94 |
| | (m, 1H), 3.37 (s, 3H), 2.28-2.25 (m, 1H), 2.24 (s, 3H), 1.84 (d, J = 22.0 Hz, |
| | 3H), 1.19-1.12 (m, 4H). |
| 69 | N-(3-cyclopropyl-1-(6-(oxetan-3-yl)pyridin-2-yl)-1H-pyrazolo[4,3- |
| | c]pyridin-6-yl)acetamide |
| | 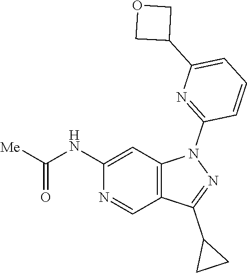 [SMILES: CC(=O)Nc1cc2c(cn1)c(C1CC1)nn2-c1cccc(C2COC2)n1] |
| | Pyridine: 2-bromo-6-(oxetan-3-yl)pyridine (Preparation 135) |
| | Prep-HPLC-C (Gradient: 33-63% MeCN) |
| | 14.6 mg, 17.9% yield as a white solid. LCMS m/z = 350.1 [M + H]⁺ |
| | ¹H NMR: (500 MHz, CDCl₃) δ ppm: 9.45 (br s, 1H), 8.74 (s, 1H), 8.10 (s, |
| | 1H), 7.83-7.76 (m, 2H), 7.09 (d, J = 7.0 Hz, 1H), 5.17-5.15 (m, 4H), 4.55- |
| | 4.49 (m, 1H), 2.32-2.26 (m, 4H), 1.24-1.15 (m, 4H). |
| 70 | N-(3-cyclopropyl-1-(4-isopropoxy-6-(3-methoxyoxetan-3-yl)pyridin-2-yl)- |
| | 1H-pyrazolo[4,3-c]pyridin-6-yl)acetamide |
| | 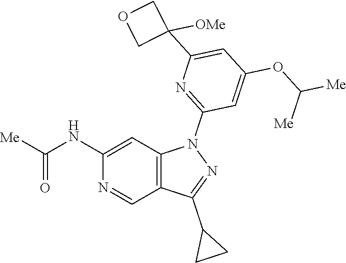 [SMILES: COC1(c2cc(OC(C)C)cc(-n3nc(C4CC4)c4cnc(NC(C)=O)cc43)n2)COC1] |
| | Pyridine: 2-bromo-4-isopropoxy-6-(3-methoxyoxetan-3-yl)pyridine |
| | (Preparation 59) |
| | Prep-HPLC-F (Gradient: 58-88% MeCN) |
| | 65.8 mg, 45.5% yield as a white solid. LCMS m/z = 438.2 [M + H]⁺ |
| | ¹H NMR: (500 MHz, CDCl₃) δ: ppm 9.33 (br s, 1H), 8.67 (s, 1H), 8.36 (s, |
| | 1H), 7.39 (d, J = 2.0 Hz, 1H), 6.78 (d, J = 1.5 Hz, 1H), 5.29-5.27 (m, 2H), |
| | 4.98-4.96 (m, 2H), 4.80-4.75 (m, 1H), 3.31 (s, 3H), 2.28-2.24 (m, 1H), |
| | 2.22 (s, 3H), 1.31 (d, J = 6.0 Hz, 6H), 1.20-1.14 (m, 4H). |
| 71 | N-(3-cyclopropyl-1-(4-(2-methoxyethoxy)-6-(3-methoxyoxetan-3- |
| | yl)pyridin-2-yl)-1H-pyrazolo[4,3-c]pyridin-6-yl)acetamide |
| | 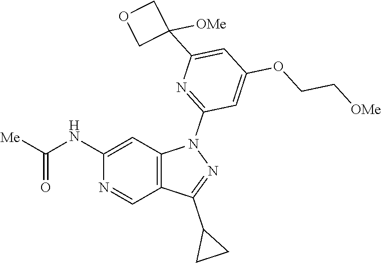 [SMILES: COCCOc1cc(-n2nc(C3CC3)c3cnc(NC(C)=O)cc32)nc(C2(OC)COC2)c1] |
| | Pyridine: 2-bromo-4-(2-methoxyethoxy)-6-(3-methoxyoxetan-3- |
| | yl)pyridine (Preparation 60) |
| | Prep-HPLC-C (Gradient: 34-64% MeCN) |
| | 36.9 mg, 29.3% yield as a white solid. LCMS m/z = 454.2 [M + H]⁺ |
| | ¹H NMR: (400 MHz, DMSO-d₆) δ ppm: 10.67 (s, 1H), 9.26 (s, 1H), 8.96 |
| | (d, J = 1.2 Hz, 1H), 7.36 (s, 1H), 6.92 (s, 1H), 5.12-5.10 (m, 2H), 4.79-4.77 |
| | (m, 2H), 4.33-4.31 (m, 2H), 3.72-3.70 (m, 2H), 3.33 (s, 3H), 3.15 (s, 3H), |
| | 2.46-2.44 (m, 1H), 2.14 (s, 3H), 1.18-1.14 (m, 4H) |
| 72 | N-(3-cyclopropyl-1-(6-methoxypyridin-2-yl)-1H-pyrazolo[4,3-c]pyridin-6- |
| | yl)acetamide |
| | 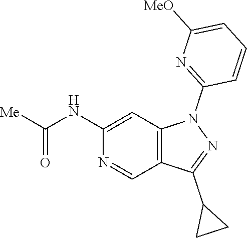 [SMILES: COc1cccc(-n2nc(C3CC3)c3cnc(NC(C)=O)cc32)n1] |
| | Pyridine: 2-bromo-6-methoxypyridine |
| | Prep-HPLC-F (Gradient: 54-84% MeCN) |
| | 51.6 mg, 15.0% yield as a white solid. LCMS /z = 324.2 [M + H]⁺ |
| | ¹H NMR: (400 MHz, CDCl₃) δ: ppm 9.44 (s, 1H), 8.73 (s, 1H), 8.15 (s, |
| | 1H), 7.67 (t, J = 8.0 Hz, 1H), 7.47 (d, J = 8.0 Hz, 1H), 6.59 (d, J = 8.0 Hz, |
| | 1H), 4.24 (s, 3H), 2.31-2.23 (m, 4H), 1.21-1.13 (m, 4H). |
| 73 | N-(3-cyclopropyl-1-(6-ethoxypyridin-2-yl)-1H-pyrazolo[4,3-c]pyridin-6- |
| | yl)acetamide |
| | 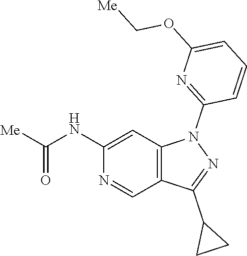 [SMILES: CCOc1cccc(-n2nc(C3CC3)c3cnc(NC(C)=O)cc32)n1] |
| | Pyridine: 2-bromo-6-ethoxypyridine |
| | Prep-HPLC-F (Gradient: 60-90% MeCN) |
| | 13.5 mg, 8.1% yield as a white solid. LCMS /z = 338.1 [M + H]⁺ |
| | ¹H NMR: (400 MHz, CDCl₃) δ: ppm 9.47 (br s, 1H), 8.67-8.66 (m, 2H), |
| | 7.67 (d, J = 8.0 Hz, 1H), 7.46 (d, J = 7.6 Hz, 1H), 6.59 (d, J = 8.0 Hz, 1H), |
| | 4.67 (t, J = 6.8 Hz, 2H), 2.30-2.23 (m, 4H), 1.51 (t, J = 7.2 Hz, 3H), 1.22- |
| | 1.15 (m, 4H). |
| 74 | N-(3-cyclopropyl-1-(6-isopropoxypyridin-2-yl)-1H-pyrazolo[4,3- |
| | c]pyridin-6-yl)acetamide |
| | 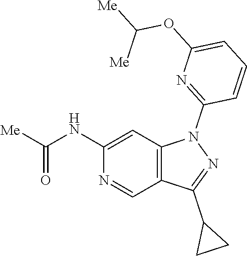 [SMILES: CC(=O)Nc1cc2c(cn1)c(C1CC1)nn2-c1cccc(OC(C)C)n1] |
| | Pyridine: 2-bromo-6-isopropoxypyridine |
| | Prep-HPLC-F (Gradient: 65-95% MeCN) |
| | 26.1 mg, 16.1% yield as a white solid. LCMS m/z = 352.1 [M + H]⁺ |
| | 1H NMR (400 MHz, CDCl₃) δ: ppm 9.30 (br s, 1H), 8.75 (br s, 1H), 8.29 |
| | (s, 1H), 7.65 (s, 1H), 7.45 (d, J = 8.0 Hz, 1H), 6.53 (d, J = 6.4 Hz, 1H), 5.72- |
| | 5.71 (m, 1H), 2.23-2.22 (m, 4H), 1.46 (d, J = 3.2 Hz, 6H), 1.20-1.15 (m, |
| | 4H). |
| 75 | N-(3-cyclopropyl-1-(6-(2-methoxyethoxy)pyridin-2-yl)-1H-pyrazolo[4,3- |
| | c]pyridin-6-yl)acetamide |
| | 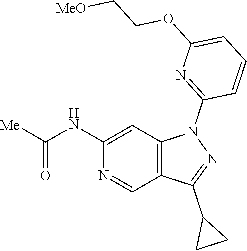 [SMILES: COCCOc1cccc(-n2nc(C3CC3)c3cnc(NC(C)=O)cc32)n1] |
| | Pyridine: 2-bromo-6-(2-methoxyethoxy)pyridine |
| | Prep-HPLC-F (Gradient: 50-80% MeCN) |
| | 70.1 mg, 44.3% yield as a white solid. LCMS m/z = 368.1 [M + H]⁺ |
| | ¹H NMR: (400 MHz, CDCl₃) δ: ppm 9.35 (br s, 1H), 9.07 (br s, 1H), 7.69 |
| | (t, J = 8.0 Hz, 1H), 7.48 (d, J = 8.0 Hz, 1H), 6.70 (d, J = 7.6 Hz, 1H), 4.80- |
| | 4.78 (m, 2H), 3.91-3.89 (m, 2H), 3.48 (s, 3H), 2.29 (s, 3H), 2.26-2.25 (m, |
| | 1H), 1.21-1.16 (m, 4H). |
| 76 | N-(3-cyclopropyl-1-(6-(2-hydroxypropan-2-yl)pyridin-2-yl)-1H- |
| | pyrazolo[4,3-c]pyridin-6-yl)acetamide |
| | 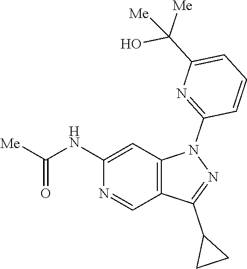 [SMILES: CC(=O)Nc1cc2c(cn1)c(C1CC1)nn2-c1cccc(C(C)(C)O)n1] |
| | Pyridine: 2-bromo-6-(2-methoxypropan-2-yl)pyridine |
| | Prep-HPLC-F (Gradient: 42-72% MeCN) |
| | 17.7 mg, 15.9% as a white solid. LCMS m/z = 366.2 [M + H]⁺ |
| | ¹H NMR (500 MHz, CDCl₃) δ ppm: 9.43 (s, 1H), 8.13 (s, 1H), 7.81-7.79 |
| | (m, 2H), 7.44-7.41 (m, 1H), 3.26 (s, 3H), 2.31-2.29 (m, 1H), 2.28 (s, 3H), |
| | 1.75 (s, 6H), 1.21-1.20 (m, 2H), 1.17-1.14 (m, 2H). |
| 77 | N-(3-cyclopropyl-1-(6-(1-methoxyethyl)pyridin-2-yl)-1H-pyrazolo[4,3- |
| | c]pyridin-6-yl)acetamide |
| | 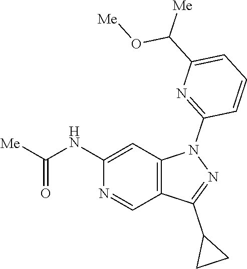 [SMILES: COC(C)c1cccc(-n2nc(C3CC3)c3cnc(NC(C)=O)cc32)n1] |
| | Pyridine: 2-bromo-6-(1-methoxyethyl)pyridine |
| | Prep-HPLC-F (Gradient: 48-78% MeCN) Detection wavelength: 220 nm) |
| | 13.7 mg, 16.9% yield as a white solid. LCMS m/z = 352.2 [M + H]⁺ |
| | ¹H NMR: (500 MHz, CDCl₃) δ: ppm 9.53 (s, 1H), 8.73 (s, 1H), 7.85-7.80 |
| | (m, 2H), 7.32 (d, J = 7.0 Hz, 1H), 4.61-4.58 (m, 1H), 3.42 (s, 3H), 2.29-2.27 |
| | (m, 4H), 1.65 (d, J = 7.0 Hz, 3H), 1.22-1.20 (m, 2H), 1.19-1.16 (m, 2H). |
| 78 | N-(1-(6-(azetidin-1-yl)pyridin-2-yl)-3-cyclopropyl-1H-pyrazolo[4,3- |
| | c]pyridin-6-yl)acetamide |
| | 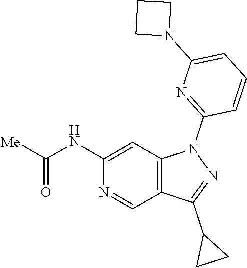 [SMILES: CC(=O)Nc1cc2c(cn1)c(C1CC1)nn2-c1cccc(N2CCC2)n1] |
| | Pyridine: 2-(azetidin-1-yl)-6-bromopyridine |
| | Prep-HPLC-F (Gradient: 52-82% MeCN) 27.3 mg, 23.9% yield as a white |
| | solid. LCMS m/z = 349.1 [M + H]⁺ |
| | ¹H NMR: (500 MHz, MeOH-d₄) δ: ppm 9.33 (s, 1H), 8.80 (s, 1H), 7.58 (t, |
| | J = 8.5 Hz, 1H), 7.12 (d, J = 8.0 Hz, 1H), 6.16 (d, J = 8.5 Hz, 1H), 4.18 (t, |
| | J = 7.5 Hz, 4H), 2.49-2.44 (m, 2H), 2.37-2.35 (m, 1H), 2.34 (s, 3H), 1.19- |
| | 1.12 (m, 4H). |
| 79 | N-(3-cyclopropyl-1-(6-(dimethylamino)pyridin-2-yl)-1H-pyrazolo[4,3- |
| | c]pyridin-6-yl)acetamide |
| | 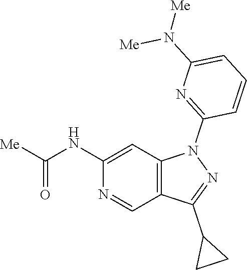 [SMILES: CC(=O)Nc1cc2c(cn1)c(C1CC1)nn2-c1cccc(N(C)C)n1] |
| | Pyridine: 6-bromo-N,N-dimethylpyridin-2-amine |
| | Prep-HPLC-C (Gradient: 43-73% MeCN) |
| | 13.5 mg, 14.5% yield as a white solid. LCMS m/z = 337.1 [M + H]⁺ |
| | ¹H NMR: (500 MHz, CDCl₃) δ ppm: 9.36 (s, 1H), 8.71 (s, 1H), 8.18 (br s, |
| | 1H), 7.54 (t, J = 7.5 Hz, 1H), 7.13 (d, J = 7.5 Hz, 1H), 6.32 (d, J = 8.0 Hz, |
| | 1H), 3.25 (s, 6H), 2.29-2.24 (m, 1H), 2.23 (s, 3H), 1.21-1.18 (m, 2H), |
| | 1.14-1.11 (m, 2H). |
| 80 | N-(3-cyclopropyl-1-(6-(2-fluoropropan-2-yl)pyridin-2-yl)-1H- |
| | pyrazolo[4,3-c]pyridin-6-yl)acetamide |
| | 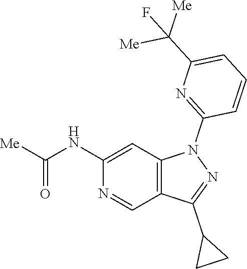 [SMILES: CC(=O)Nc1cc2c(cn1)c(C1CC1)nn2-c1cccc(C(C)(C)F)n1] |
| | Pyridine: 2-bromo-6-(2-fluoropropan-2-yl)pyridine (Preparation 132) |
| | Prep-HPLC-C (Gradient: 50-80% MeCN) |
| | 50.4 mg, 44.4% yield as a white solid. LCMS m/z = 354.1 [M + H]⁺ |
| | ¹H NMR: (400 MHz, DMSO-d₆) δ ppm: 10.70 (s, 1H), 9.36 (s, 1H), 8.97 |
| | (s, 1H), 8.02 (t, J = 8.0 Hz, 1H), 7.81 (d, J = 8.0 Hz, 1H), 7.40 (d, J = 7.6 Hz, |
| | 1H), 2.50-2.44 (m, 1H), 2.15 (s, 3H), 1.85 (s, 3H), 1.79 (s, 3H), 1.16-1.13 |
| | (m, 4H). |
| 81 | N-(3-ethyl-1-(6-(2-methoxyethoxy)pyridin-2-yl)-1H-pyrazolo[4,3- |
| | c]pyridin-6-yl)acetamide |
| | 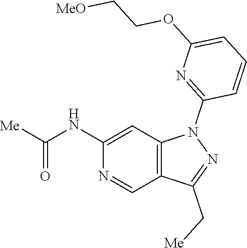 [SMILES: CCc1nn(-c2cccc(OCCOC)n2)c2cc(NC(C)=O)ncc12] |
| | Pyridine: 2-bromo-6-(2-methoxyethoxy)pyridine |
| | Acetamide: N-(3-ethyl-1H-pyrazolo[4,3-c]pyridin-6-yl)acetamide |
| | (Preparation 9) |
| | Prep-HPLC-F (Gradient: 48-78% MeCN) |
| | 62.1 mg, 40.6% yield as a white solid. LCMS m/z = 356.1 [M + H]⁺ |
| | ¹H NMR: (500 MHz, CDCl₃) δ: ppm 9.39 (s, 1H), 8.69 (s, 1H), 8.14 (br s, |
| | 1H), 7.69 (t, J = 8.0 Hz, 1H), 7.53 (d, J = 7.5 Hz, 1H), 6.68 (d, J = 8.0 Hz, |
| | 1H), 4.83-4.81 (m, 2H), 3.93-3.91 (m, 2H), 3.49 (s, 3H), 3.05 (q, J = 7.5 Hz, |
| | 2H), 2.23 (s, 3H), 1.47 (t, J = 7.5 Hz, 3H). |

### Examples 82 and 83: (S)—N-(1-(6-(3-fluorotetrahydrofuran-3-yl)pyridin-2-yl)-1H-pyrazolo[4,3-c]pyridin-6-yl)acetamide and (R)—N-(1-(6-(3-fluorotetrahydrofuran-3-yl)pyridin-2-yl)-1H-pyrazolo[4,3-c]pyridin-6-yl)acetamide

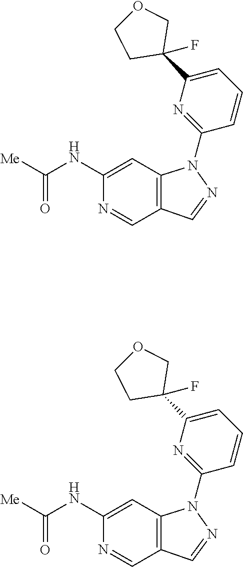 [SMILES: CC(=O)Nc1cc2c(cn1)cnn2-c1cccc([C@@]2(F)CCOC2)n1 ; CC(=O)Nc1cc2c(cn1)cnn2-c1cccc([C@]2(F)CCOC2)n1]

Step A: N-(1-(6-(3-fluorotetrahydrofuran-3-yl)pyridin-2-yl)-1H-pyrazolo[4,3-c]pyridin-6-yl)acetamide was obtained as a white solid, 32 mg, 11.5% yield from 2-bromo-6-(3-fluorotetrahydrofuran-3-yl)pyridine (Preparation 63) and N-(1H-pyrazolo[4,3-c]pyridin-6-yl)acetamide (Preparation 2), following a similar procedure to that described in Example 44. LCMS m/z=342.1 [M+H]⁺

Step B: N-(1-(6-(3-fluorotetrahydrofuran-3-yl)pyridin-2-yl)-1H-pyrazolo[4,3-c]pyridin-6-yl)acetamide was further separated by prep-SFC (Condition: 0.1% NH₃·H₂O EtOH; Column: DAICEL CHIRALPAK AD(250 mm*30 mm,10 um); Begin B: 40%; End B: 40%; Flow Rate (ml/min): 70) to give:

Peak 1, Enantiomer 1 (27.4 mg, 42.2% yield) as a white solid. LCMS m/z=342.1 [M+H]⁺. ¹H NMR: (400 MHz, CDCl₃) δ: ppm 9.37 (s, 1H), 8.78 (d, J=0.8 Hz, 1H), 8.23 (s, 1H), 8.13 (br s, 1H), 7.97 (d, J=7.2 Hz, 1H), 7.91 (t, J=7.6 Hz, 1H), 7.56 (d, J=6.8 Hz, 1H), 4.52-4.51 (m, 1H), 4.37-4.26 (m, 3H), 3.22-3.10 (m, 1H), 2.61-2.53 (m, 1H), 2.25 (s, 3H).

Peak 2, Enantiomer 2 (26.1 mg, 40.2% yield) as a white solid. LCMS m/z=342.1 [M+H]⁺. ¹H NMR: (400 MHz, CDCl₃) δ: ppm 9.37 (s, 1H), 8.79 (s, 1H), 8.23 (s, 1H), 8.10 (br s, 1H), 7.97 (d, J=6.8 Hz, 1H), 7.91 (t, J=7.2 Hz, 1H), 7.56 (d, J=7.2 Hz, 1H), 4.52-4.51 (m, 1H), 4.37-4.26 (m, 3H), 3.23-3.10 (m, 1H), 2.60-2.54 (m, 1H), 2.26 (s, 3H).

### Examples 84 and 85: (S)—N-(1-(6-(3-fluorotetrahydrofuran-3-yl)-4-methoxypyridin-2-yl)-3-methyl-1H-pyrazolo[4,3-c]pyridin-6-yl)acetamide and (R)—N-(1-(6-(3-fluorotetrahydrofuran-3-yl)-4-methoxypyridin-2-yl)-3-methyl-1H-pyrazolo[4,3-c]pyridin-6-yl)acetamide

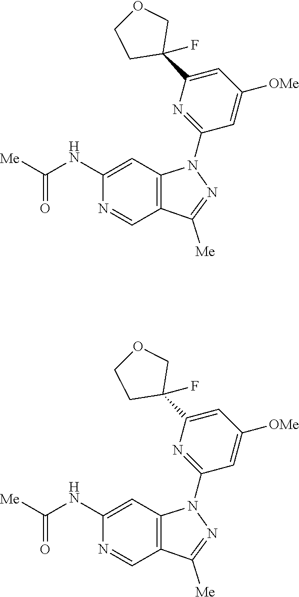 [SMILES: COc1cc(-n2nc(C)c3cnc(NC(C)=O)cc32)nc([C@@]2(F)CCOC2)c1 ; COc1cc(-n2nc(C)c3cnc(NC(C)=O)cc32)nc([C@]2(F)CCOC2)c1]

Step A: N-(1-(6-(3-fluorotetrahydrofuran-3-yl)-4-methoxypyridin-2-yl)-3-methyl-1H-pyrazolo[4,3-c]pyridin-6-yl)acetamide was obtained as a white solid, 90 mg, 8.9% yield, from 2-bromo-6-(3-fluorotetrahydrofuran-3-yl)-4-methoxypyridine (Preparation 65) and N-(3-methyl-1H-pyrazolo[4,3-c]pyridin-6-yl)acetamide (Preparation 6), following a similar procedure to that described in Example 44. ¹H NMR: (500 MHz, CDCl₃) δ ppm: 9.32 (s, 1H), 8.68 (s, 1H), 8.11 (s, 1H), 7.42 (d, J=2.0 Hz, 1H), 7.06 (d, J=1.5 Hz, 1H), 4.49-4.48 (m, 1H), 4.33-4.21 (m, 3H), 3.98 (s, 3H), 3.20-3.09 (m, 1H), 2.66 (s, 3H), 2.54-2.51 (m, 1H), 2.25 (s, 3H).

Step B: N-(1-(6-(3-Fluorotetrahydrofuran-3-yl)-4-methoxypyridin-2-yl)-3-methyl-1H-pyrazolo[4,3-c]pyridin-6-yl)acetamide (Example X, 90 mg, 0.233 mmol) was further separated by SFC (Condition: 0.10% NH₃·H₂O EtOH; Column: DAICEL CHIRALPAK AD (250 mm*30 mm,10 um); Begin B: 35%; End B: 35%; Flow Rate (ml/min): 70) to give: Peak 1, enantiomer 1 (26.5 mg, 29.4% yield) as a white solid. LCMS m/z=386.1 [M+H]⁺. ¹H NMR: (500 MHz, CDCl₃) δ ppm: 9.29 (s, 1H), 8.66 (s, 1H), 8.23 (s, 1H), 7.42 (s, 1H), 7.06 (s, 1H), 4.49-4.48 (m, 1H), 4.33-4.22 (m, 3H), 3.98 (s, 3H), 3.19-3.09 (m, 1H), 2.65 (s, 3H), 2.55-2.47 (m, 1H), 2.24 (s, 3H); and

Peak 2, enantiomer 2, (29.8 mg, 33.1% yield) as a white solid. LCMS m/z=386.1 [M+H]⁺. ¹H NMR: (400 MHz, CDCl₃) δ ppm: 9.29 (s, 1H), 8.66 (s, 1H), 8.14 (s, 1H), 7.42 (s, 1H), 7.06 (s, 1H), 4.49-4.48 (m, 1H), 4.31-4.21 (m, 3H), 3.98 (s, 3H), 3.20-3.08 (m, 1H), 2.65 (s, 3H), 2.55-2.45 (m, 1H), 2.24 (s, 3H).

### Example 86: N-(3-cyclopropyl-1-(6-(1,1-difluoroethyl)pyridin-2-yl)-1H-pyrazolo[4,3-c]pyridin-6-yl)acetamide

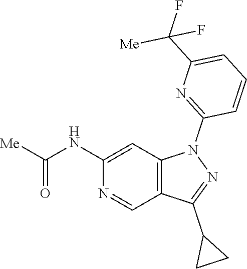 [SMILES: CC(=O)Nc1cc2c(cn1)c(C1CC1)nn2-c1cccc(C(C)(F)F)n1]

A mixture of N-(3-cyclopropyl-1H-pyrazolo[4,3-c]pyridin-6-yl)acetamide (Preparation 11, 160 mg, 0.740 mmol), 2-bromo-6-(1,1-difluoroethyl)pyridine (328.6 mg, 1.48 mmol), (1R,2R)—N1,N2-dimethylcyclohexane-1,2-diamine (21.0 mg, 0.148 mmol) and CuI (14.1 mg, 0.074 mmol) in dioxane (6.0 mL) was purged with N₂ and the reaction was heated at 100° C. for 1 h. The cooled mixture was diluted with DCM, the resulting solid filtered off and the filtrate concentrated in vacuo. The crude was purified by chromatography on silica gel (0-100% EtOAc/heptanes), followed by HPLC purification (10-90% water/MeCN with 0.1% TFA modifier). The fraction was then neutralized with sat'd aqueous NaHCO₃, extracted with EtOAc, the organic layer was then separated, dried and concentrated to give N-(3-cyclopropyl-1-(6-(1,1-difluoroethyl)pyridin-2-yl)-1H-pyrazolo[4,3-c]pyridin-6-yl)acetamide (72 mg, 27% yield) as a white powder. LCMS m/z=358.1 [M+H]^{+ 1}H NMR (400 MHz, CDCl₃) δ 9.43 (s, 1H), 8.74 (s, 1H), 8.51 (br s, 1H), 8.03 (d, J=8.28 Hz, 1H), 7.92 (t, J=8.03 Hz, 1H), 7.60-7.47 (m, 1H), 2.39-2.26 (m, 7H), 1.23 (dd, J=2.26, 5.27 Hz, 2H), 1.18 (td, J=2.67, 8.22 Hz, 2H).

### Example 87: N-(1-(6-(tetrahydrofuran-3-yl)pyridin-2-yl)-1H-pyrazolo[4,3-c]pyridin-6-yl)acetamide

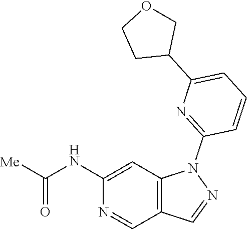 [SMILES: CC(=O)Nc1cc2c(cn1)cnn2-c1cccc(C2CCOC2)n1]

Step A: To a solution of N-(1H-pyrazolo[4,3-c]pyridin-6-yl)acetamide (Preparation 2, 200 mg, 1.14 mmol) in dioxane (5 mL) was added 2,6-dibromopyridine (268.9 mg, 1.14 mmol), K₂CO₃ (470.7 mg, 3.41 mmol), N1,N2-dimethylethane-1,2-diamine (20.0 mg, 0.227 mmol) and CuI (86.5 mg, 0.454 mmol) and the reaction was stirred at 90° C. under N₂ for 2 h. The cooled mixture was concentrated under reduced pressure and the residue was purified by chromatography on silica gel (PE/EtOAc=1/2) to give N-(1-(6-bromopyridin-2-yl)-1H-pyrazolo[4,3-c]pyridin-6-yl)acetamide (145 mg, 38.5% yield) as a white solid. 1H NMR (500 MHz, CDCl₃) δ ppm: 9.50 (br s, 1H), 8.78 (s, 1H), 8.22 (s, 1H), 8.18 (s, 1H), 7.95 (d, J=8.5 Hz, 1H), 7.69 (t, J=8.0 Hz, 1H), 7.38 (d, J=8.0 Hz, 1H), 2.29 (s, 3H).

Step B: A mixture of N-(1-(6-bromopyridin-2-yl)-1H-pyrazolo[4,3-c]pyridin-6-yl)acetamide (135 mg, 0.406 mmol), furan-3-ylboronic acid (45.5 mg, 0.406 mmol), Pd(dppf)Cl₂ (29.7 mg, 0.041 mmol) and Cs₂CO₃ (264.9 mg, 0.813 mmol) in dioxane (2.5 mL) and H₂O (0.5 mL) was degassed and purged with N₂ then the mixture was stirred at 90° C. for 6 h under N₂. The reaction was concentrated under reduced pressure and the residue was purified by chromatography on silica gel (PE/EtOAc=1/3) to give N-(1-(6-(furan-3-yl)pyridin-2-yl)-1H-pyrazolo[4,3-c]pyridin-6-yl)acetamide (100 mg, 77% yield) as a yellow solid. 1H NMR: (500 MHz, CDCl₃) δ ppm: 9.66 (br s, 1H), 8.79 (s, 1H), 8.63 (s, 1H), 8.23 (s, 1H), 8.14 (s, 1H), 7.90-7.82 (m, 2H), 7.59-7.58 (m, 1H), 7.36 (d, J=7.0 Hz, 1H), 7.15 (s, 1H), 2.30 (s, 3H).

Step C: To a solution of N-(1-(6-(furan-3-yl)pyridin-2-yl)-1H-pyrazolo[4,3-c]pyridin-6-yl)acetamide (100 mg, 0.313 mmol) in MeOH (5 mL) was added Pd/C (166.6 mg, 0.157 mmol, 10% purity) under N₂ atmosphere. The suspension was degassed and purged with H₂ (3×) and the reaction was stirred under H₂ (15 Psi) at 20° C. for 16 h. The mixture was filtered and the filtrate was concentrated under reduced pressure. The mixture was purified by Prep-HPLC-C (Gradient: 25-55% MeCN) to give N-(1-(6-(tetrahydrofuran-3-yl)pyridin-2-yl)-1H-pyrazolo[4,3-c]pyridin-6-yl)acetamide (31.5 mg, 31.1% yield) as a white solid. LCMS m/z=324.2 [M+H]⁺ 1H NMR (400 MHz, CDCl₃) δ ppm: 9.46 (br s, 1H), 8.78 (s, 1H), 8.21-8.19 (m, 2H), 7.85 (d, J=8.0 Hz, 1H), 7.78 (t, J=8.0 Hz, 1H), 7.12 (d, J=7.6 Hz, 1H), 4.35-4.31 (m, 1H), 4.26-4.24 (m, 1H), 4.06-4.02 (m, 2H), 3.72-3.70 (m, 1H), 2.5-2.49 (m, 2H), 2.26 (s, 3H).

### Example 88: N-(1-(4-methoxy-6-(tetrahydrofuran-3-yl)pyridin-2-yl)-3-methyl-1H-pyrazolo[4,3-c]pyridin-6-yl)acetamide

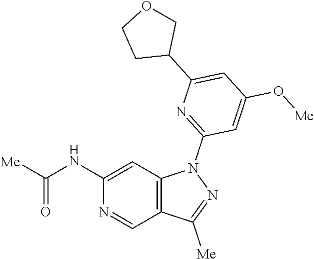 [SMILES: COc1cc(C2CCOC2)nc(-n2nc(C)c3cnc(NC(C)=O)cc32)c1]

Step A: To a solution of 2-bromo-6-(furan-3-yl)-4-methoxypyridine (Preparation 122, 40.1 mg, 0.158 mmol) and N-(3-methyl-1H-pyrazolo[4,3-c]pyridin-6-yl)acetamide (Preparation 6, 30 mg, 158 mmol) in dioxane (3 mL) was added N,N′-dimethylethane-1,2-diamine (2.8 mg, 0.032 mmol), CuI (12.0 mg, 0.063 mmol) and K₂CO₃ (43.6 mg, 0.315 mmol) and the reaction was stirred at 90° C. for 2 h under N₂. The mixture was concentrated under reduced pressure and the residue was purified by chromatography on silica gel (PE/EtOAc=0/1) to give N-(1-(6-(furan-3-yl)-4-methoxypyridin-2-yl)-3-methyl-1H-pyrazolo[4,3-c]pyridin-6-yl)acetamide (28 mg, 48.9% yield) as a yellow solid. LCMS m/z=364.1 [M+H]⁺. ¹H NMR (400 MHz, CDCl₃) δ: ppm 9.60 (s, 1H), 8.67 (s, 1H), 8.62 (s, 1H), 8.17 (br s, 1H), 7.56-7.55 (m, 1H), 7.41-7.38 (m, 1H), 7.07 (s, 1H), 6.86 (d, J=2.0 Hz, 1H), 3.99 (s, 3H), 2.66 (s, 3H), 2.29 (s, 3H).

Step B: To a solution of N-(1-(6-(furan-3-yl)-4-methoxypyridin-2-yl)-3-methyl-1H-pyrazolo[4,3-c]pyridin-6-yl)acetamide (28 mg, 0.077 mmol) in MeOH (3 mL) was added Pd/C (24.6 mg, 0.023 mmol, 10% purity) and the reaction was stirred at 30° C. for 3 h under 30 Psi of H₂. The mixture was concentrated under reduced pressure and the residue was purified by Prep-HPLC-F (Gradient: 30-60% MeCN) to give N-(1-(4-methoxy-6-(tetrahydrofuran-3-yl)pyridin-2-yl)-3-methyl-1H-pyrazolo[4,3-c]pyridin-6-yl)acetamide (2.9 mg, 10.2% yield) as a white solid. LCMS m/z=368.2 [M+H]⁺. ¹H NMR: (400 MHz, CDCl₃) δ: ppm 9.39 (s, 1H), 8.67 (s, 1H), 8.04 (s, 1H), 7.35 (d, J=2.0 Hz, 1H), 6.63 (d, J=2.0 Hz, 1H), 4.32-4.22 (m, 2H), 4.03-3.99 (m, 2H), 3.94 (s, 3H), 3.63-3.58 (m, 1H), 2.66 (s, 3H), 2.52-2.44 (m, 2H), 2.25 (s, 3H).

### Example 89 and 90: N-(1-(4-chloro-6-(1,1-difluoroethyl)pyridin-2-yl)-3-methyl-1H-pyrazolo[4,3-c]pyridin-6-yl)acetamide and N-(1-(2-chloro-6-(1,1-difluoroethyl)pyridin-4-yl)-3-methyl-1H-pyrazolo[4,3-c]pyridin-6-yl)acetamide

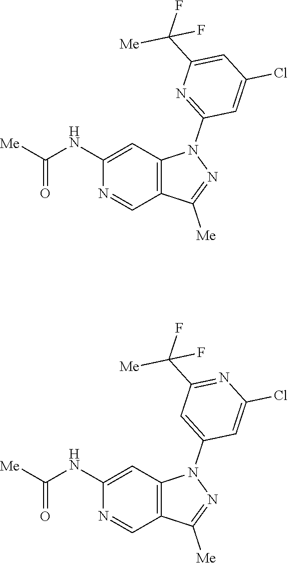 [SMILES: CC(=O)Nc1cc2c(cn1)c(C)nn2-c1cc(Cl)cc(C(C)(F)F)n1 ; CC(=O)Nc1cc2c(cn1)c(C)nn2-c1cc(Cl)nc(C(C)(F)F)c1]

To a solution of 2,4-dichloro-6-(1,1-difluoroethyl)pyridine (Preparation 127, 100 mg, 0.472 mmol) in DMF (2 mL) was added N-(3-methyl-1H-pyrazolo[4,3-c]pyridin-6-yl)acetamide (Preparation 6, 89.7 mg, 0.472 mmol) and Cs₂CO₃ (307.3 mg, 0.943 mmol) and the reaction was stirred at 100° C. for 16 h. The cooled mixture was concentrated and was purified by Prep-HPLC-C (Gradient: 50-85% MeCN) to give N-(1-(2-chloro-6-(1,1-difluoroethyl)pyridin-4-yl)-3-methyl-1H-pyrazolo[4,3-c]pyridin-6-yl)acetamide (82.3 mg, 47.7% yield) as a yellow solid. LCMS m/z=366.1 [M+H]⁺. ¹H NMR: (400 MHz, CDCl₃) δ ppm: 8.82 (s, 1H), 8.75 (s, 1H) 8.38 (br s, 1H), 8.09 (s, 1H), 7.88 (s, 1H), 2.69 (s, 3H), 2.31 (s, 3H), 2.08 (t, J=18.8 Hz, 3H).

and N-(1-(4-chloro-6-(1,1-difluoroethyl)pyridin-2-yl)-3-methyl-1H-pyrazolo[4,3-c]pyridin-6-yl)acetamide (10.4 mg, 6.0% yield) as a yellow solid. LCMS m/z=366.1 [M+H]⁺. ¹H NMR: (400 MHz, CDCl₃) δ ppm: 9.40 (s, 1H), 8.68 (s, 1H), 8.32 (br s, 1H), 8.10 (s, 1H), 7.53 (s, 1H), 2.67 (s, 3H), 2.35-2.26 (m, 6H).

### Example 91: N-(1-(6-(3-(fluoromethyl)tetrahydrofuran-3-yl)pyridin-2-yl)-1H-pyrazolo[4,3-c]pyridin-6-yl)acetamide

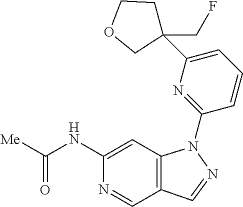 [SMILES: CC(=O)Nc1cc2c(cn1)cnn2-c1cccc(C2(CF)CCOC2)n1]

To a solution of N-(1H-pyrazolo[4,3-c]pyridin-6-yl)acetamide (Preparation 2, 96.7 mg, 0.55 mmol) in DMF (2.0 mL) was added 2-fluoro-6-(3-(fluoromethyl)tetrahydrofuran-3-yl)pyridine (Preparation 80, 72.9 mg, 0.37 mmol) and K₂CO₃ (101.2 mg, 0.73 mmol) and the reaction stirred at 100° C. for 12 h. The cooled mixture was filtered and purified by Prep-HPLC-C (Gradient: 27-57% MeCN) to give N-(1-(6-(3-(fluoromethyl)tetrahydrofuran-3-yl)pyridin-2-yl)-1H-pyrazolo[4,3-c]pyridin-6-yl)acetamide (21.5 mg, 16.5% yield) as a white solid. LCMS m/z=356.2 [M+H]⁺. ¹H NMR: (500 MHz, CDCl₃) δ ppm: 9.41 (s, 1H), 8.78 (s, 1H), 8.22 (s, 1H), 8.12 (s, 1H), 7.93 (d, J=8.0 Hz, 1H), 7.85-7.82 (m, 1H), 7.20 (d, J=8.0 Hz, 1H), 4.92-4.81 (m, 2H), 4.40-4.38 (m, 1H), 4.20-4.07 (m, 3H), 2.55-2.48 (m, 2H), 2.26 (s, 3H). Further elution provided N-(2-(6-(3-(fluoromethyl)tetrahydrofuran-3-yl)pyridin-2-yl)-2H-pyrazolo[4,3-c]pyridin-6-yl)acetamide (14.7 mg, 11.3% yield) as a white solid. LCMS m/z=356.2 [M+H]⁺. ¹H NMR: (500 MHz, CDCl₃) δ ppm: 9.21 (s, 1H), 8.98 (d, J=1.0 Hz, 1H), 8.44 (s, 1H), 8.20 (d, J=8.0 Hz, 1H), 8.00 (s, 1H), 7.94-7.91 (m, 1H), 7.39 (d, J=7.0 Hz, 1H), 4.70 (d, J=47.5 Hz, 2H), 4.20-4.16 (m, 2H), 4.12-4.02 (m, 2H), 2.48-2.45 (m, 1H), 2.32-2.28 (m, 1H), 2.25 (s, 3H).

### Example 92: N-(1-(6-(3-(difluoromethyl)tetrahydrofuran-3-yl)pyridin-2-yl)-1H-pyrazolo[4,3-c]pyridin-6-yl)acetamide

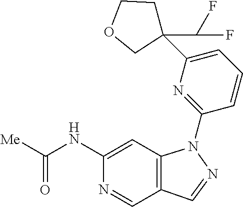 [SMILES: CC(=O)Nc1cc2c(cn1)cnn2-c1cccc(C2(C(F)F)CCOC2)n1]

To a solution of N-(1H-pyrazolo[4,3-c]pyridin-6-yl)acetamide (Preparation 2, 40.6 mg, 0.230 mmol) in DMF (2.0 mL) was added 2-(3-(difluoromethyl)tetrahydrofuran-3-yl)-6-fluoropyridine (Preparation 86, 50.0 mg, 0.230 mmol) and K₂CO₃ (63.6 mg, 0.46 mmol) and the reaction was stirred at 100° C. for 6 h. The cooled mixture was filtered and purified by Prep-HPLC-C (Gradient: 30-60% MeCN) to give N-(1-(6-(3-(difluoromethyl)tetrahydrofuran-3-yl)pyridin-2-yl)-1H-pyrazolo[4,3-c]pyridin-6-yl)acetamide (7.71 mg, 9.0% yield) as a white solid. LCMS m/z=374.2 [M+H]^{+ 1}H NMR: (500 MHz, CDCl₃) δ ppm: 9.37 (s, 1H), 8.78 (s, 1H), 8.22 (s, 1H), 8.11 (s, 1H), 7.96 (d, J=8.5 Hz, 1H), 7.88-7.84 (m, 1H), 7.26 (d, J=8.0 Hz, 1H), 6.47 (t, J=56.0 Hz, 1H), 4.60-4.59 (m, 1H), 4.34-4.31 (m, 1H), 4.13-4.01 (m, 2H), 2.78-2.72 (m, 1H), 2.62-2.59 (m, 1H), 2.26 (s, 3H). Further elution provided N-(2-(6-(3-(difluoromethyl)tetrahydrofuran-3-yl)pyridin-2-yl)-2H-pyrazolo[4,3-c]pyridin-6-yl)acetamide (6.34 mg, 6.8% yield) as a white solid. LCMS m/z=374.0 [M+H]^{+ 1}H NMR: (500 MHz, CDCl₃) δ ppm: 9.18 (s, 1H), 8.97 (s, 1H), 8.42 (s, 1H), 8.23 (d, J=8.0 Hz, 1H), 7.96-7.92 (m, 2H), 7.41 (d, J=8.0 Hz, 1H), 6.20 (t, J=56.0 Hz, 1H), 4.38-4.30 (m, 1H), 4.29-4.27 (m, 1H), 4.08-3.97 (m, 2H), 2.60-2.53 (m, 2H), 2.52 (s, 3H).

### Example 93: N-(3-methyl-1-(6-(tetrahydrofuran-3-yl)pyridin-2-yl)-1H-pyrazolo[4,3-c]pyridin-6-yl)acetamide

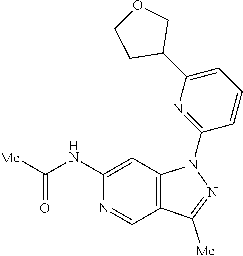 [SMILES: CC(=O)Nc1cc2c(cn1)c(C)nn2-c1cccc(C2CCOC2)n1]

To a solution of N-(3-methyl-1H-pyrazolo[4,3-c]pyridin-6-yl)acetamide (Preparation 6, 50 mg, 0.263 mmol) in DMF (3 mL) was added 2-fluoro-6-(tetrahydrofuran-3-yl)pyridine (48.4 mg, 0.289 mmol) and K₂CO₃ (72.7 mg, 0.526 mmol) and the reaction was stirred at 120° C. for 20 h. The cooled mixture was purified by Prep-HPLC-C (Gradient: 29-59% MeCN) to give N-(3-methyl-1-(6-(tetrahydrofuran-3-yl)pyridin-2-yl)-1H-pyrazolo[4,3-c]pyridin-6-yl)acetamide (16.5 mg, 18.6% yield) as a white solid. LCMS m/z=338.2 [M+H]^{+ 1}H NMR: (500 MHz, CDCl₃) δ ppm: 9.39 (s, 1H), 8.67 (s, 1H), 8.17 (s, 1H), 7.81-7.72 (m, 2H), 7.07 (d, J=7.5 Hz, 1H), 4.35-4.30 (m, 1H), 4.25-4.23 (m, 1H), 4.06-4.01 (m, 2H), 3.71-3.66 (m, 1H), 2.66 (s, 3H), 2.54-2.48 (m, 2H), 2.26 (s, 3H).

### Example 94: N-(3-methyl-1-(4-methyl-6-(tetrahydrofuran-3-yl)pyridin-2-yl)-1H-pyrazolo[4,3-c]pyridin-6-yl)acetamide

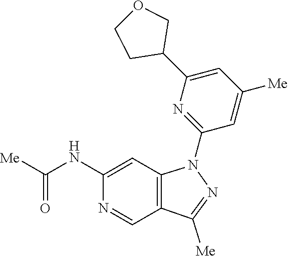 [SMILES: CC(=O)Nc1cc2c(cn1)c(C)nn2-c1cc(C)cc(C2CCOC2)n1]

N-(3-Methyl-1-(4-methyl-6-(tetrahydrofuran-3-yl)pyridin-2-yl)-1H-pyrazolo[4,3-c]pyridin-6-yl)acetamide was obtained as a white solid, 11.9 mg, 12.9% yield, from N-(3-methyl-1H-pyrazolo[4,3-c]pyridin-6-yl)acetamide (Preparation 6) and 2-fluoro-4-methyl-6-(tetrahydrofuran-3-yl)pyridine (Preparation 121) following a similar procedure to that described in Example 93. LCMS m/z=338.2 [M+H]⁺. ¹H NMR: (400 MHz, CDCl₃) δ ppm: 9.38 (s, 1H), 8.67 (s, 1H), 8.08 (s, 1H), 7.64 (s, 1H), 6.90 (s, 1H), 4.33-4.22 (m, 2H), 4.04-3.99 (m, 2H), 3.66-3.61 (m, 1H), 2.66 (s, 3H), 2.53-2.48 (m, 2H), 2.41 (s, 3H), 2.25 (s, 3H).

### Example 95 and 96: N-(1-(2-chloro-6-(1,1-difluoroethyl)pyridin-4-yl)-3-cyclopropyl-1H-pyrazolo[4,3-c]pyridin-6-yl)acetamide and N-(1-(4-chloro-6-(1,1-difluoroethyl)pyridin-2-yl)-3-cyclopropyl-1H-pyrazolo[4,3-c]pyridin-6-yl)acetamide

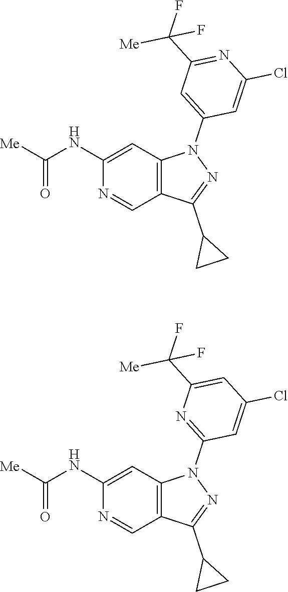 [SMILES: CC(=O)Nc1cc2c(cn1)c(C1CC1)nn2-c1cc(Cl)nc(C(C)(F)F)c1 ; CC(=O)Nc1cc2c(cn1)c(C1CC1)nn2-c1cc(Cl)cc(C(C)(F)F)n1]

To a solution of 2,4-dichloro-6-(1,1-difluoroethyl)pyridine (Preparation 127, 100 mg, 0.472 mmol) in DMF (3 mL) was added N-(3-cyclopropyl-1H-pyrazolo[4,3-c]pyridin-6-yl)acetamide (Preparation 11, 102 mg, 0.472 mmol) and K₂CO₃ (130.4 mg, 0.943 mmol) and the reaction was stirred at 100° C. for 5 h. The mixture was concentrated in vacuo and purified by Prep-HPLC-C (Gradient: 53-83% MeCN) to give N-(1-(2-chloro-6-(1,1-difluoroethyl)pyridin-4-yl)-3-cyclopropyl-1H-pyrazolo[4,3-c]pyridin-6-yl)acetamide (3 mg, 1.62% yield) as a white solid. LCMS m/z=392.1 [M+H]^{+ 1}H NMR: (400 MHz, CDCl) δ ppm: 8.80-8.79 (m, 2H), 8.47 (br s, 1H), 8.03 (s, 1H), 7.82 (s, 1H), 2.29-2.27 (m, 4H), 2.07 (t, J=18.4 Hz, 3H), 1.26-1.20 (m, 4H) and N-(1-(4-chloro-6-(1,1-difluoroethyl)pyridin-2-yl)-3-cyclopropyl-1H-pyrazolo[4,3-c]pyridin-6-yl)acetamide (3 mg, 1.62% yield) as a white solid. LCMS m/z=392.1 [M+H]⁺

¹H NMR: (400 MHz, CDCl₃) δ ppm: 9.50 (s, 1H), 9.11 (br s, 1H), 8.74 (s, 1H), 8.05 (s, 1H), 7.54-7.52 (m, 1H), 2.34-2.23 (m, 7H), 1.25-1.21 (m, 4H).

### Example 97: (S)—N-(1-(6-(1,1-difluoroethyl)pyridin-2-yl)-3-(3-(dimethylamino) pyrrolidin-1-yl)-1H-pyrazolo[4,3-c]pyridin-6-yl)acetamide

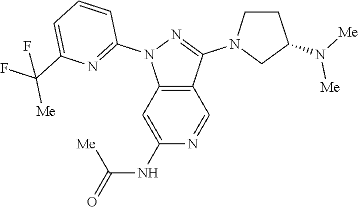 [SMILES: CC(=O)Nc1cc2c(cn1)c(N1CC[C@H](N(C)C)C1)nn2-c1cccc(C(C)(F)F)n1]

To a solution of (S)-1-(6-chloro-1-(6-(1,1-difluoroethyl)pyridin-2-yl)-1H-pyrazolo[4,3-c]pyridin-3-yl)-N,N-dimethylpyrrolidin-3-amine (Preparation 147, 50 mg, 0.123 mmol) in dioxane (3 mL) was added acetamide (8.0 mg, 0.135 mmol), Cs₂CO₃ (80.1 mg, 0.246 mmol) and BrettPhos Pd G₃ (11.1 mg, 12 μmol) and the reaction was stirred at 100° C. for 2 h under N₂. The mixture was purified by Prep-HPLC-L (Gradient:47-77% MeCN) to give (S)—N-(1-(6-(1,1-difluoroethyl)pyridin-2-yl)-3-(3-(dimethylamino)pyrrolidin-1-yl)-1H-pyrazolo[4,3-c]pyridin-6-yl)acetamide (20.1 mg, 38.1% yield) as a light-yellow solid. LCMS m/z=430.2 [M+H]^{+ 1}H NMR: (400 MHz, CDCl₃) δ ppm: 9.38 (s, 1H), 8.72 (s, 1H), 8.02 (s, 1H), 7.95-7.93 (m, 1H), 7.88-7.84 (m, 1H), 7.43-7.41 (m, 1H), 3.96-3.92 (m, 2H), 3.76-3.73 (m, 1H), 3.56-3.51 (m, 1H), 3.00-2.90 (m, 1H), 2.37 (s, 6H), 2.31 (t, J=19.2 Hz, 3H), 2.29-2.28 (m, 1H), 2.26 (s, 3H), 2.06-2.00 (m, 1H).

### Example 98: (R)—N-(1-(6-(1,1-difluoroethyl)pyridin-2-yl)-3-(3-(dimethylamino) pyrrolidin-1-yl)-1H-pyrazolo[4,3-c]pyridin-6-yl)acetamide

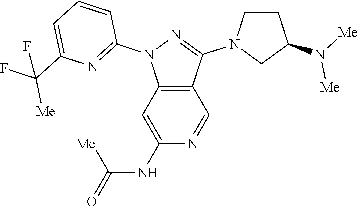 [SMILES: CC(=O)Nc1cc2c(cn1)c(N1CC[C@@H](N(C)C)C1)nn2-c1cccc(C(C)(F)F)n1]

To a solution of (R)-1-(6-chloro-1-(6-(1,1-difluoroethyl)pyridin-2-yl)-1H-pyrazolo[4,3-c]pyridin-3-yl)-N,N-dimethylpyrrolidin-3-amine (Preparation 148, 30 mg, 0.074 mmol) in dioxane (1 mL) was added acetamide (6.5 mg, 0.111 mmol), Cs₂CO₃ (48.1 mg, 0.147 mmol) and BrettPhos Pd G3 (6.7 mg, 7.37 umol) and the reaction was stirred at 100° C. for 3 h under N₂. The cooled mixture was concentrated and was purified by Prep-HPLC-L (Gradient:50-75% MeCN) to give (R)—N-(1-(6-(1,1-difluoroethyl)pyridin-2-yl)-3-(3-(dimethylamino)pyrrolidin-1-yl)-1H-pyrazolo[4,3-c]pyridin-6-yl)acetamide (8.2 mg, 25.9% yield) as a yellow solid. LCMS m/z=430.1 [M+H]^{+ 1}H NMR: (400 MHz, CDCl₃) δ: ppm 9.38 (s, 1H), 8.72 (s, 1H), 8.02 (s, 1H), 7.95 (d, J=8.4 Hz, 1H), 7.88 (t, J=8.4 Hz, 1H), 7.43 (d, J=6.8 Hz, 1H) 3.96-3.93 (m, 2H), 3.76-3.74 (m, 1H), 3.56-3.52 (m, 1H), 2.96-2.93 (m, 1H), 2.36-2.26 (m, 13H), 2.26-2.01 (m, 1H).

### Example 99: N-(1-(4-(1,1-difluoroethyl)pyridin-2-yl)-3-morpholino-1H-pyrrolo[3,2-c]pyridin-6-yl)acetamide

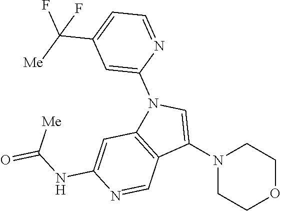 [SMILES: CC(=O)Nc1cc2c(cn1)c(N1CCOCC1)cn2-c1cc(C(C)(F)F)ccn1]

To a solution of 4-(6-chloro-1-(4-(1,1-difluoroethyl)pyrimidin-4-yl)-1H-pyrrolo[3,2-c]pyridin-3-yl)morpholine (Preparation 151, 32.0 mg, 0.084 mmol) and acetamide (7.5 mg, 0.127 mmol) in dioxane (3 mL) was added Cs₂CO₃ (82.57 mg, 0.253 mmol) and BrettPhos Pd G₃ (7.66 mg, 8.45 umol) and the reaction was stirred at 100° C. for 2 h under N₂. The reaction was concentrated under reduced pressure to give the residue, which was purified by Prep-HPLC-C (Gradient: 30-60% MeCN) to give impure product (15.0 mg, crude) as a white solid. The crude product was purified by prep-TLC (EtOAc) and further purified by Prep-HPLC-C (Gradient: 33-63% MeCN) to give N-(1-(4-(1,1-difluoroethyl)pyridin-2-yl)-3-morpholino-1H-pyrrolo[3,2-c]pyridin-6-yl)acetamide (4.8 mg, 14.2% yield) as a light red solid. LCMS m/z=402.1 [M+H]⁺

### Example 100: N-(1-(6-(1,1-difluoroethyl)pyridin-2-yl)-3-morpholino-1H-pyrazolo[4,3-c]pyridin-6-yl)-3-methoxypropanamide

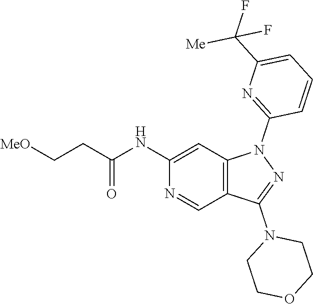 [SMILES: COCCC(=O)Nc1cc2c(cn1)c(N1CCOCC1)nn2-c1cccc(C(C)(F)F)n1]

A mixture of 4-(6-chloro-1-(4-(1,1-difluoroethyl)pyridin-2-yl)-1H-pyrazolo[4,3-c]pyridin-3-yl)morpholine (Preparation 151, 60 mg, 0.158 mmol), 3-methoxypropanamide (32.6 mg, 0.316 mmol), BrettPhos Pd G3 (28.6 mg, 0.032 mmol) and Cs₂CO₃ (103.0 mg, 0.316 mmol) in toluene (3 mL) was stirred at 100° C. for 2 h under N₂. The mixture was concentrated and purified by Prep-TLC (PE/EtOAc=1/4) to give a white solid. This was purified by Prep-HPLC-F (Gradient: 45-75%) to give N-(1-(6-(1,1-difluoroethyl)pyridin-2-yl)-3-morpholino-1H-pyrazolo[4,3-c]pyridin-6-yl)-3-methoxypropanamide (5.1 mg, 7.2% yield) as a white solid. LCMS m/z=447.2 [M+H]^{+ 1}H NMR: (400 MHz, CDCl₃) δ: ppm 9.50 (s, 1H), 9.28 (br s, 1H), 8.75 (s, 1H), 7.95-7.91 (m, 1H), 7.90-7.88 (m, 1H), 7.48 (d, J=7.2 Hz, 1H), 3.96-3.93 (m, 4H), 3.79-3.77 (m, 2H), 3.61-3.58 (m, 4H), 3.47 (s, 3H), 2.75-2.72 (m, 2H), 2.30 (t, J=18.8 Hz, 1H).

### Example 101: N-(3-cyclopropyl-1-(6-(1,1-difluoroethyl)pyridin-2-yl)-1H-pyrazolo[4,3-c]pyridin-6-yl)-3-methoxypropanamide

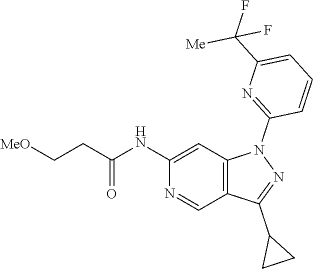 [SMILES: COCCC(=O)Nc1cc2c(cn1)c(C1CC1)nn2-c1cccc(C(C)(F)F)n1]

N-(3-Cyclopropyl-1-(6-(1,1-difluoroethyl)pyridin-2-yl)-1H-pyrazolo[4,3-c]pyridin-6-yl)-3-methoxypropanamide was obtained as a white solid, 27.4 mg, 17.6% yield, from 3-methoxypropanamide and 6-chloro-3-cyclopropyl-1-(6-(1,1-difluoroethyl)pyridin-2-yl)-1H-pyrazolo[4,3-c]pyridine (Preparation 142), following a similar procedure to that described in Example 100. LCMS m/z=402.1 [M+H]⁺. ¹H NMR: (500 MHz, CDCl₃) δ: ppm 10.40-8.99 (m, 2H), 8.01 (d, J=8.0 Hz, 1H), 7.92 (t, J=8.0 Hz, 1H), 7.54 (d, J=7.5 Hz, 1H), 3.86-3.83 (m, 2H), 3.48 (s, 3H), 2.79-2.78 (m, 2H), 2.34-2.26 (m, 4H), 1.22-1.18 (m, 4H).

### Example 102: N-(1-(6-(3-methoxytetrahydrofuran-3-yl)pyridin-2-yl)-1H-pyrazolo[4,3-c]pyridin-6-yl)acetamide

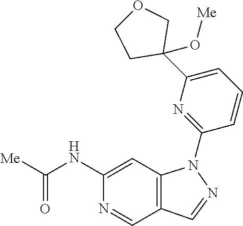 [SMILES: COC1(c2cccc(-n3ncc4cnc(NC(C)=O)cc43)n2)CCOC1]

To a solution of 1-(6-(3-methoxytetrahydrofuran-3-yl)pyridin-2-yl)-1H-pyrazolo[4,3-c]pyridin-6-amine (Preparation 152, 100 mg, 0.321 mmol) in DMF (5 mL) was added TEA (44.7 μL) and the solution cooled to 0° C. AcCl (34.3 μL) was added and the reaction mixture was stirred at 50° C. for 3 h. The mixture was purified by Prep-HPLC-C (Gradient: 23-53% MeCN) to give N-(1-(6-(3-methoxytetrahydrofuran-3-yl)pyridin-2-yl)-1H-pyrazolo[4,3-c]pyridin-6-yl)acetamide (14.8 mg, 13% yield) as white solid. LCMS m/z=354.2 [M+H]⁺. 1H NMR: (400 MHz, CDCl₃) δ ppm: 8.82 (s, 1H), 8.76 (s, 1H), 8.28 (d, J=3.6 Hz, 1H), 8.16 (s, 1H), 7.96 (d, J=8.0 Hz, 1H), 7.45 (d, J=7.6 Hz, 1H), 6.66 (d, J=4.0 Hz, 1H), 4.22-4.08 (m, 4H), 3.25 (s, 3H), 2.69-2.63 (m, 1H), 2.52 (s, 3H), 2.45-2.42 (m, 1H).

### Example 103: N-(7-(5-(3-methoxytetrahydrofuran-3-yl)pyridin-3-yl)-7H-pyrrolo[2,3-d]pyrimidin-2-yl)acetamide

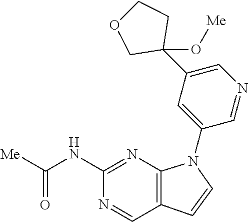 [SMILES: COC1(c2cncc(-n3ccc4cnc(NC(C)=O)nc43)c2)CCOC1]

To a solution of 7-(5-(3-methoxytetrahydrofuran-3-yl)pyridin-3-yl)-7H-pyrrolo[2,3-d]pyrimidin-2-amine hydrochloride (Preparation 154, 142 mg, 0.456 mmol) in Ac₂O (232.8 mg, 2.28 mmol) was added TEA (55.4 mg, 0.547 mmol) and the reaction was stirred at 70° C. for 2 h. The pH of the reaction was adjusted to 7-8 with ammonium hydroxide. The mixture was concentrated and was purified by Prep-HPLC-F (Gradient: 15-45% MeCN) to give N-(7-(5-(3-methoxytetrahydrofuran-3-yl)pyridin-3-yl)-7H-pyrrolo[2,3-d]pyrimidin-2-yl)acetamide (64.8 mg, 40.2% yield) as a white solid. LCMS m/z=354.2 [M+H]⁺. ¹H NMR (400 MHz, CDCl₃) δ ppm: 8.93 (d, J=2.0 Hz, 1H), 8.90 (s, 1H), 8.65 (d, J=1.6 Hz, 1H), 8.36 (s, 1H), 8.30 (s, 1H), 7.47 (d, J=3.6 Hz, 1H), 6.75 (d, J=3.6 Hz, 1H), 4.27-4.24 (m, 1H), 4.18-4.12 (m, 2H), 3.97 (d, J=9.2 Hz, 1H), 3.20 (s, 3H), 2.58-2.56 (m, 1H), 2.51 (s, 3H), 2.41-2.35 (m, 1H).

### Example 104: N-(1-(6-(1-amino-2,2,2-trifluoroethyl)pyridin-2-yl)-1H-pyrazolo[4,3-c]pyridin-6-yl)acetamide

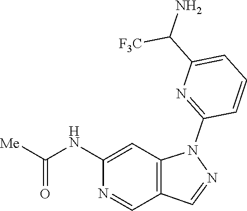

To a solution of tert-butyl (1-(6-(6-acetamido-1H-pyrazolo[4,3-c]pyridin-1-yl)pyridin-2-yl)-2,2,2-trifluoroethyl)carbamate (Preparation 138, 66.9 mg, 0.149 mmol) in DCM (2.0 mL) was added HCl/dioxane (4 M, 2.0 mL) and the reaction was stirred at 20° C. for 1 h. The mixture was filtered and purified by Prep-HPLC-K (Gradient: 15-45% MeCN) to give N-(1-(6-(1-amino-2,2,2-trifluoroethyl)pyridin-2-yl)-1H-pyrazolo[4,3-c]pyridin-6-yl)acetamide (26.7 mg, 51.3% yield) as a white solid. LCMS m/z=351.2 [M+H]^{+ 1}H NMR: (400 MHz, DMSO-d₆) δ ppm: 10.74 (s, 1H), 9.49 (s, 1H), 8.96 (s, 1H), 8.56 (s, 1H), 8.09-8.05 (m, 1H), 7.99 (d, J=8.0 Hz, 1H), 7.49 (d, J=7.6 Hz, 1H), 4.81-4.76 (m, 1H), 2.16 (s, 3H).

### Example 105: N-(3-cyclopropyl-1-(4-(tetrahydrofuran-3-yl)pyrimidin-2-yl)-1H-pyrazolo[4,3-c]pyridin-6-yl)acetamide

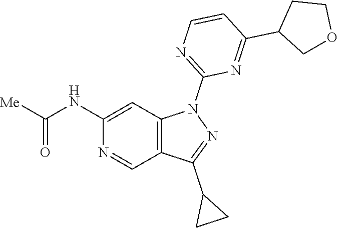 [SMILES: CC(=O)Nc1cc2c(cn1)c(C1CC1)nn2-c1nccc(C2CCOC2)n1]

To a solution of N-(3-cyclopropyl-1-(4-(furan-3-yl)pyrimidin-2-yl)-1H-pyrazolo[4,3-c]pyridin-6-yl)acetamide (Preparation 171, 60 mg, 0.167 mmol) in MeOH (5 mL) was added Pd/C (17.7 mg, 0.017 mmol, 10% purity) and the resulting mixture was stirred at 20° C. for 2 h under H₂ balloon (15 Psi). The mixture was evaporated to dryness in vacuo and the residue purified by prep-HPLC-C (Gradient=29-59% MeCN) to give N-(3-cyclopropyl-1-(4-(tetrahydrofuran-3-yl)pyrimidin-2-yl)-1H-pyrazolo[4,3-c]pyridin-6-yl)acetamide as a white solid (18.7 mg, 31%). LCMS m/z=365.1 [M+H]⁺; ¹H NMR (400 MHz, CDCl₃) δ: 9.55 (s, 1H), 9.41 (br s, 1H), 8.77 (d, 1H), 8.71 (s, 1H), 7.15 (d, 1H), 4.31-4.21 (m, 2H), 4.07-4.00 (m, 2H), 3.73-3.71 (m, 1H), 2.56-2.42 (m, 3H), 2.32 (s, 3H), 1.32-1.21 (m, 4H).

### Example 106: N-(3-cyclopropyl-1-(4-(3-fluorotetrahydrofuran-3-yl)pyrimidin-2-yl)-1H-pyrazolo[4,3-c]pyridin-6-yl)acetamide

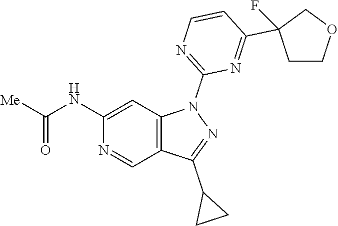 [SMILES: CC(=O)Nc1cc2c(cn1)c(C1CC1)nn2-c1nccc(C2(F)CCOC2)n1]

The title compound was prepared as a white solid (30 mg, 43%) from N-(3-cyclopropyl-1-(4-(furan-3-yl)pyrimidin-2-yl)-1H-pyrazolo[4,3-c]pyridin-6-yl)acetamide (Preparation 171) and 2-chloro-4-(3-fluorotetrahydrofuran-3-yl)pyrimidine (Preparation 168) using an analogous method to that described for Example 105. LCMS m/z=383.1 [M+H]⁺; ¹H NMR (400 MHz, CDCl₃) δ: 9.28 (s, 1H), 8.88 (d, 1H), 8.69 (s, 1H), 8.54 (s, 1H), 7.51-7.49 (m, 1H), 4.52-4.49 (m, 1H), 4.33-4.27 (m, 2H), 4.26-4.24 (m, 1H), 3.17-3.05 (m, 1H), 2.60-2.56 (m, 1H), 2.38-2.33 (m, 1H), 2.24 (s, 3H), 1.30-1.27 (m, 2H), 1.19-1.16 (m, 2H).

### Example 107: N-(1-(4-(3-methoxytetrahydrofuran-3-yl)pyrimidin-2-yl)-1H-pyrazolo[4,3-c]pyridin-6-yl)acetamide

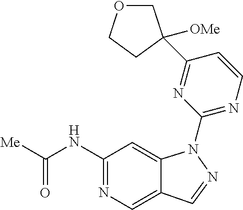 [SMILES: COC1(c2ccnc(-n3ncc4cnc(NC(C)=O)cc43)n2)CCOC1]

A mixture of N-(1H-pyrazolo[4,3-c]pyridin-6-yl)acetamide (Preparation 2, 82.1 mg, 0.466 mmol), 2-chloro-4-(3-methoxytetrahydrofuran-3-yl)pyrimidine (Preparation 166, 100 mg, 0.466 mmol) and K₂CO₃ (128.8 mg, 0.932 mmol) in DMF (3.0 mL) was stirred at 100° C. for 4 h. The mixture was filtered and the filtrate purified by prep-HPLC-C (Gradient=12-42% MeCN) to give N-(1-(4-(3-methoxytetrahydrofuran-3-yl)pyrimidin-2-yl)-1H-pyrazolo[4,3-c]pyridin-6-yl)acetamide as a white solid (109.2 mg, 66%). LCMS m/z=355.2 [M+H]⁺; ¹H NMR (400 MHz, CDCl₃) δ: 9.36 (s, 1H), 8.88 (d, 1H), 8.80 (s, 1H), 8.34 (s, 1H), 8.21 (s, 1H), 7.49 (d, 1H), 4.35-4.31 (m, 3H), 4.27-4.24 (m, 1H), 3.35 (s, 3H), 3.04-2.96 (m, 1H), 2.57-2.54 (m, 1H), 2.27 (s, 3H).

### Example 108: N-(1-(3-(1,1-difluoroethyl)pyrazin-2-yl)-1H-pyrazolo[4,3-c]pyridin-6-yl)acetamide

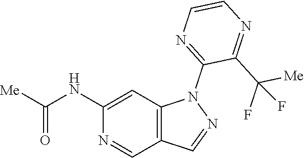 [SMILES: CC(=O)Nc1cc2c(cn1)cnn2-c1nccnc1C(C)(F)F]

Cs₂CO₃ (124.1 mg, 0.381 mmol) was added to a solution of N-(1H-pyrazolo[4,3-c]pyridin-6-yl)acetamide (Preparation 2, 33.5 mg, 0.190 mmol) and 2-chloro-3-(1,1-difluoroethyl)pyrazine (Preparation 173, 34.0 mg, 0.190 mmol) in DMF (2 mL) at 25° C. and the mixture stirred at 70° C. for 3 h. The mixture was purified by Prep-HPLC-C (Gradient=18-48% MeCN) to give N-(2-(3-(1,1-difluoroethyl)pyrazin-2-yl)-2H-pyrazolo[4,3-c]pyridin-6-yl)acetamide and N-(1-(3-(1,1-difluoroethyl)pyrazin-2-yl)-1H-pyrazolo[4,3-c]pyridin-6-yl)acetamide as a white solid (8.3 mg, 13.7%). LCMS m/z=318.9 [M+H]⁺; ¹H NMR (500 MHz, CDCl₃) δ: 8.82 (s, 1H), 8.74 (s, 1H), 8.68 (d, 1H), 8.53 (s, 1H), 8.26 (s, 1H), 8.17 (s, 1H), 2.26-2.19 (m, 6H).

### Example 109: N-(3-cyclopropyl-1-(4-(1,1-difluoroethyl)-6-methylpyrimidin-2-yl)-1H-pyrazolo[4,3-c]pyridin-6-yl)acetamide

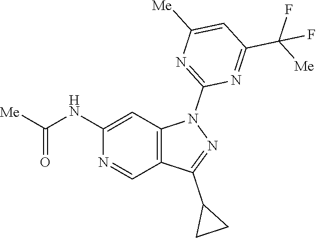 [SMILES: CC(=O)Nc1cc2c(cn1)c(C1CC1)nn2-c1nc(C)cc(C(C)(F)F)n1]

To a solution of N-(3-cyclopropyl-1H-pyrazolo[4,3-c]pyridin-6-yl)acetamide (Preparation 11, 1.6 g, 7.40 mmol) in DMF (20 mL) was added 2-chloro-4-(1,1-difluoroethyl)-6-methylpyrimidine (Preparation 180, 1.4 g, 7.40 mmol) and Cs₂CO₃ (4.8 g, 14.80 mmol) and the resulting mixture was stirred at 70° C. for 12 h. The reaction mixture was concentrated and the residue purified by chromatography (SiO₂, 5-50% EtOAc/PE) to give N-(3-cyclopropyl-1-(4-(1,1-difluoroethyl)-6-methylpyrimidin-2-yl)-1H-pyrazolo[4,3-c]pyridin-6-yl)acetamide as a yellow solid (1.13 g, 41%). LCMS m/z=373.1 [M+H]⁺; ¹H NMR (500 MHz, CDCl₃) δ: 9.41 (s, 1H), 8.69 (s, 1H), 8.26 (br s, 1H), 7.36 (s, 1H), 2.75 (s, 3H), 2.43-2.39 (m, 1H), 2.29-2.22 (m, 6H), 1.30-1.29 (m, 2H), 1.21-1.18 (m, 2H).

### Example 110-158

The title compounds were prepared from the appropriate pyrazolo[4,3-c]pyridine (SM) and chloropyrimidine (RCl) using an analogous method to that described for Example 109. Alternatives conditions as noted in the table. SM-1: N-(1H-pyrazolo[4,3-c]pyridin-6-yl)acetamide (Preparation 2); SM-2: N-(3-methyl-1H-pyrazolo[4,3-c]pyridin-6-yl)acetamide (Preparation 6); SM-3: N-(3-ethyl-1H-pyrazolo[4,3-c]pyridin-6-yl)acetamide (Preparation 9); SM-4: N-(3-cyclopropyl-1H-pyrazolo[4,3-c]pyridin-6-yl)acetamide (Preparation 11); SM-5: N-(3-(4-methylpiperazin-1-yl)-1H-pyrazolo[4,3-c]pyridin-6-yl)acetamide (Preparation 159); SM-6: N-(3-(3-cyanoazetidin-1-yl)-1H-pyrazolo[4,3-c]pyridin-6-yl)acetamide (Preparation 161); SM-7: N-(3-cyclopropyl-1H-pyrrolo[3,2-c]pyridin-6-yl)acetamide (Preparation 21); SM-8: N-(3-(4-methoxypiperidin-1-yl)-1H-pyrazolo[4,3-c]pyridin-6-yl)acetamide trifluoroacetate (Preparation 162); SM-9: N-(3-(pyrrolidin-1-yl)-1H-pyrazolo[4,3-c]pyridin-6-yl)acetamide (Preparation 160).

| Example | |
|---|---|
| No | Name/Structure/RCl/Data |
| 110 | N-(1-(2-(1,1-difluoroethyl)pyrimidin-4-yl)-1H-pyrazolo[4,3-c]pyridin-6- |
| | yl)acetamide |
| | 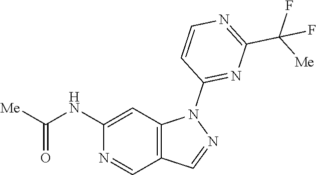 [SMILES: CC(=O)Nc1cc2c(cn1)cnn2-c1ccnc(C(C)(F)F)n1] |
| | SM-1; RCl: 4-chloro-2-(1,1-difluoroethyl)pyrimidine (Preparation 184) |
| | HPLC-C (22-52% MeCN); White solid (8.4 mg, 6%); LCMS m/z = 319.1 |
| | [M + H]⁺; ¹H NMR (400 MHz, CDCl₃) δ: 9.67 (s, 1H), 8.92 (d, 1H), 8.81 (s, |
| | 1H), 8.76 (s, 1H), 8.35 (s, 1H), 8.03 (d, 1H), 2.36-2.26 (m, 6H). |
| 111^{A} | N-(1-(2-(1,1-difluoroethyl)pyrimidin-4-yl)-3-methyl-1H-pyrazolo[4,3- |
| | c]pyridin-6-yl)acetamide |
| | 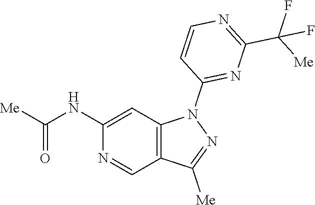 [SMILES: CC(=O)Nc1cc2c(cn1)c(C)nn2-c1ccnc(C(C)(F)F)n1] |
| | SM-2; RCl: 4-chloro-2-(1,1-difluoroethyl)pyrimidine (Preparation 184); |
| | HPLC-C (25-42% MeCN); White solid (4.9 mg, 3%); LCMS m/z = 333.1 |
| | [M + H]⁺; ¹H NMR (400 MHz, CDCl₃) δ: 9.58 (s, 1H), 8.87-8.86 (m, 1H), |
| | 8.71 (s, 1H), 8.58 (s, 1H), 7.97 (d, 1H), 2.68 (s, 3H), 2.35-2.26 (m, 6H). |
| 112 | N-(1-(6-(1, 1-difluoroethyl)pyrazin-2-yl)-3-methyl-1H-pyrazolo[4,3- |
| | c]pyridin-6-yl)acetamide |
| | 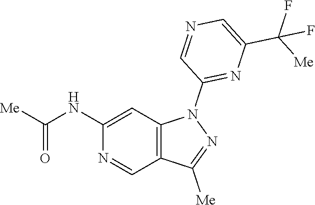 [SMILES: CC(=O)Nc1cc2c(cn1)c(C)nn2-c1cncc(C(C)(F)F)n1] |
| | SM-2; RCl: 2-chloro-6-(1,1-difluoroethyl)pyrazine (Preparation 176) |
| | HPLC-F (38-68% MeCN); White solid (14.5 mg, 3.9%); LCMS m/z = |
| | 333.1 [M + H]⁺; ¹H NMR (500 MHz, CDCl₃) δ: 9.43 (s, 1H), 9.36 (s, 1H), |
| | 8.80 (s, 1H), 8.72 (s, 1H), 8.10 (s, 1H), 2.69 (s, 3H), 2.33 (t, 3H), 2.27 (s, |
| | 3H). |
| 113 | N-(1-(6-(1,1-difluoroethyl)pyrazin-2-yl)-1H-pyrazolo[4,3-c]pyridin-6- |
| | yl)acetamide |
| | 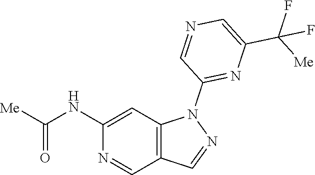 [SMILES: CC(=O)Nc1cc2c(cn1)cnn2-c1cncc(C(C)(F)F)n1] |
| | SM-1; RCl: 2-chloro-6-(1,1-difluoroethyl)pyrazine (Preparation 176) |
| | HPLC-F (35-65% MeCN); Yellow solid (4.3 mg, 1.2%); LCMS m/z = |
| | 319.1 [M + H]⁺; ¹H NMR (500 MHz, CDCl₃) δ: 9.49 (s, 1H), 9.43 (s, 1H), |
| | 8.85 (s, 1H), 8.83 (s, 1H), 8.31 (s, 1H), 8.12 (s, 1H), 2.34 (t, 1H), 2.28 (s, |
| | 3H). |
| 114 | N-(1-(6-(1,1-difluoroethyl)pyrazin-2-yl)-3-ethyl-1H-pyrazolo[4,3- |
| | c]pyridin-6-yl)acetamide |
| | 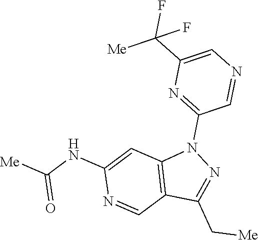 [SMILES: CCc1nn(-c2cncc(C(C)(F)F)n2)c2cc(NC(C)=O)ncc12] |
| | SM-3; RCl: 2-chloro-6-(1,1-difluoroethyl)pyrazine (Preparation 176) |
| | HPLC-C: (38-68% MeCN); White solid (14.6 mg, 3.8%); LCMS m/z = |
| | 347.1 [M + H]⁺; ¹H NMR (400 MHz, CDCl₃) δ: 9.45 (s, 1H), 9.37 (s, 1H), |
| | 8.79 (s, 1H), 8.75 (s, 1H), 8.14 (br s, 1H), 3.09 (q, 2H), 2.33 (t, 3H), 2.27 (s, |
| | 3H), 1.50 (t, 3H). |
| 115 | N-(3-cyclopropyl-1-(6-(1,1-difluoroethyl)pyrazin-2-yl)-1H-pyrazolo[4,3- |
| | c]pyridin-6-yl)acetamide |
| | 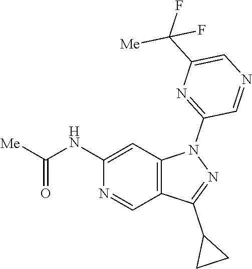 [SMILES: CC(=O)Nc1cc2c(cn1)c(C1CC1)nn2-c1cncc(C(C)(F)F)n1] |
| | SM-4; RCl: 2-chloro-6-(1,1-difluoroethyl)pyrazine (Preparation 176) |
| | HPLC-F (47-77% MeCN) followed by HPLC-C (Gradient = 41-71% |
| | MeCN); White solid (3.5 mg, 1%). LCMS m/z = 359.2 [M + H]⁺; ¹H NMR |
| | (400 MHz, CDCl₃) δ: 9.38 (s, 1H), 9.35 (s, 1H), 8.77 (s, 2H), 8.12 (br s, |
| | 1H), 2.37-2.27 (m, 7H), 1.26-1.19 (m, 4H). |
| 116 | N-(3-cyclopropyl-1-(4-(3-fluorooxetan-3-yl)pyrimidin-2-yl)-1H- |
| | pyrazolo[4,3-c]pyridin-6-yl)acetamide |
| | 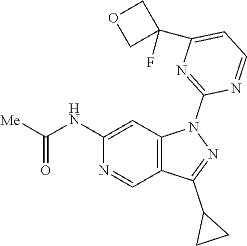 [SMILES: CC(=O)Nc1cc2c(cn1)c(C1CC1)nn2-c1nccc(C2(F)COC2)n1] |
| | SM-4; RCl: 2-chloro-4-(3-fluorooxetan-3-yl)pyrimidine (Preparation 169) |
| | HPLC-C (20-50% MeCN); Yellow solid (28.5 mg, 15%); LCMS m/z = |
| | 369.1 [M + H]⁺; ¹H NMR (400 MHz, CDCl₃) δ: 9.39 (s, 1H), 8.91 (d, 1H), |
| | 8.74 (s, 1H), 8.09 (br s, 1H), 7.38 (d, 1H), 5.43-5.35 (m, 2H), 5.15-5.07 (m, |
| | 2H), 2.41-2.37 (m, 1H), 2.25 (s, 3H), 1.33-1.22 (m, 2H), 1.21-1.18 (m, 2H). |
| 117 | N-(3-(3-cyanoazetidin-1-yl)-1-(6-(1,1-difluoroethyl)pyrazin-2-yl)-1H- |
| | pyrazolo[4,3-c]pyridin-6-yl)acetamide |
| | 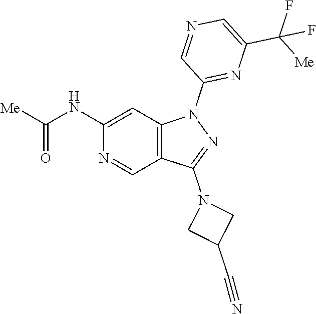 [SMILES: CC(=O)Nc1cc2c(cn1)c(N1CC(C#N)C1)nn2-c1cncc(C(C)(F)F)n1] |
| | SM-6; RCl: 2-chloro-6-(1,1-difluoroethyl)pyrazine (Preparation 176) |
| | HPLC-C (28-58% MeCN); Yellow solid (6 mg, 3.9%); LCMS m/z = 399.0 |
| | [M + H]⁺; ¹H NMR (400 MHz, CDCl₃) δ: 9.37 (s, 1H), 9.27 (s, 1H), 8.74 (s, |
| | 1H), 8.52 (s, 1H), 8.31 (s, 1H), 4.66-4.51 (m, 4H), 3.88-3.75 (m, 1H), 2.38- |
| | 2.24 (m, 6H). |
| 118 | N-(1-(6-(1,1-difluoroethyl)pyrazin-2-yl)-3-(4-methylpiperazin-1-yl)-1H- |
| | pyrazolo[4,3-c]pyridin-6-yl)acetamide |
| | 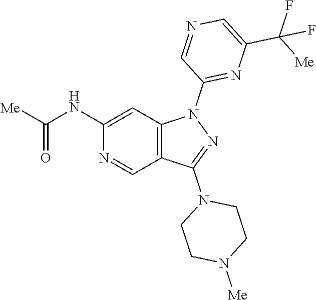 [SMILES: CC(=O)Nc1cc2c(cn1)c(N1CCN(C)CC1)nn2-c1cncc(C(C)(F)F)n1] |
| | SM-5; RCl: 2-chloro-6-(1,1-difluoroethyl)pyrazine (Preparation 176) |
| | Purification: Poured into water and solid collected; White solid (25.8 mg, |
| | 28%); LCMS m/z = 417.2 [M + H]⁺; ¹H NMR (500 MHz, CDCl₃) δ: 9.37 (s, |
| | 1H), 9.29 (s, 1H), 8.77 (s, 1H), 8.70 (s, 1H), 8.16 (s, 1H), 3.71 (br s, 4H), |
| | 2.70 (br s, 4H), 2.43 (s, 3H), 2.36-2.24 (m, 6H). |
| 119 | N-(1-(4-(1,1-difluoroethyl)pyrimidin-2-yl)-1H-pyrazolo[4,3-c]pyridin-6- |
| | yl)acetamide |
| | 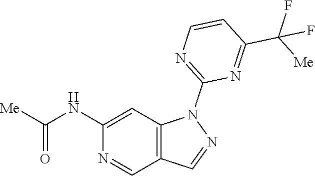 [SMILES: CC(=O)Nc1cc2c(cn1)cnn2-c1nccc(C(C)(F)F)n1] |
| | SM-1: RCl: 2-chloro-4-(1,1-difluoroethyl)pyrimidine (Preparation 178) |
| | SiO₂, 0-50% EtOAc/PE; White solid (43.2 mg, 61%); LCMS m/z = 319.1 |
| | [M + H]⁺; ¹H NMR (500 MHz, CDCl₃) δ: 9.45 (s, 1H), 9.03 (d, 1H), 8.81 (s, |
| | 1H), 8.36 (s, 1H), 8.13 (br s, 1H), 7.56 (d, 1H), 2.30 (t, J = 19.5 Hz, 3H), |
| | 2.28 (s, 3H). |
| 120 | N-(1-(4-(1,1-difluoroethyl)pyrimidin-2-yl)-3-methyl-1H-pyrazolo[4,3- |
| | c]pyridin-6-yl)acetamide |
| | 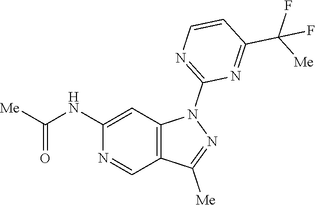 [SMILES: CC(=O)Nc1cc2c(cn1)c(C)nn2-c1nccc(C(C)(F)F)n1] |
| | SM-2; RCl: 2-chloro-4-(1,1-difluoroethyl)pyrimidine (Preparation 178) |
| | HPLC-G (25-55% MeCN); White solid (6.1 mg, 8%). LCMS m/z = 333.2 |
| | [M + H]⁺; ¹H NMR (400 MHz, CDCl₃) δ: 9.39 (s, 1H), 9.00 (d, 1H), 8.72 (s, |
| | 1H), 8.24 (br s, 1H), 7.51 (d, 1H), 2.72 (s, 3H), 2.30 (t, 3H), 2.28 (s, 3H). |
| 121 | N-(3-cyclopropyl-1-(4-(1,1-difluoroethyl)pyrimidin-2-yl)-1H-pyrazolo[4,3- |
| | c]pyridin-6-yl)acetamide |
| | 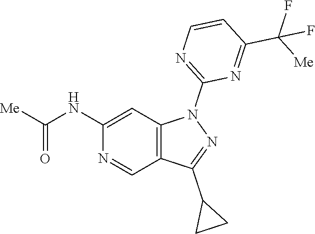 [SMILES: CC(=O)Nc1cc2c(cn1)c(C1CC1)nn2-c1nccc(C(C)(F)F)n1] |
| | SM-4; RCl: 2-chloro-4-(1,1-difluoroethyl)pyrimidine (Preparation 178). |
| | SiO₂, 66% EtOAc/PE; White solid (208 mg, 30%); LCMS m/z = 359.1 |
| | [M + H]⁺; ¹H NMR (400 MHz, CDCl₃) δ: 9.39 (s, 1H), 9.00 (d, 1H), 8.73 (s, |
| | 1H), 8.20 (br s, 1H), 7.49 (d, 1H), 2.38-2.34 (m, 1H), 2.30-2.25 (m, 6H), |
| | 1.31-1.29 (m, 2H), 1.21-1.19 (m, 2H). |
| 122^{A} | N-(1-(4-(1,1-difluoroethyl)pyrimidin-2-yl)-3-ethyl-1H-pyrazolo[4,3- |
| | c]pyridin-6-yl)acetamide |
| | 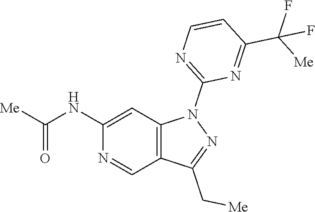 [SMILES: CCc1nn(-c2nccc(C(C)(F)F)n2)c2cc(NC(C)=O)ncc12] |
| | SM-3; RCl: 2-chloro-4-(1,1-difluoroethyl)pyrimidine (Preparation 178). |
| | HPLC-F (31-61% MeCN); White solid (19.4 mg, 13%). LCMS m/z = |
| | 347.1 [M + H]⁺; ¹H NMR (400 MHz, CDCl₃) δ: 9.44 (s, 1H), 9.01 (d, 1H), |
| | 8.76 (s, 1H), 8.53-8.48 (m, 1H), 7.52 (d, 1H), 3.17-3.12 (m, 2H), 2.35-2.25 |
| | (m, 6H), 1.51-1.42 (m, 3H). |
| 123 | N-(3-cyclopropyl-1-(4-(1,1-difluoroethyl)pyrimidin-2-yl)-1H-pyrrolo[3,2- |
| | c]pyridin-6-yl)acetamide |
| | 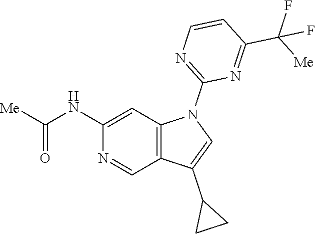 [SMILES: CC(=O)Nc1cc2c(cn1)c(C1CC1)cn2-c1nccc(C(C)(F)F)n1] |
| | SM-7; RCl: 2-chloro-4-(1,1-difluoroethyl)pyrimidine (Preparation 178) |
| | HPLC-C (40-70% MeCN); White solid (33.5 mg, 50%). LCMS m/z = |
| | 358.1 [M + H]⁺; ¹H NMR (400 MHz, CDCl₃) δ: 9.44 (s, 1H), 8.85 (d, 1H), |
| | 8.63 (s, 1H), 8.20 (br s, 1H), 7.87 (s, 1H), 7.40 (d, 1H), 2.25 (s, 3H), 2.24 (t, |
| | 1H), 1.96-1.99 (m, 1H), 0.99-1.01 (m, 2H), 0.76-0.79 (m, 2H). |
| 124 | N-(3-cyclopropyl-1-(4-(1,1-difluoroethyl)-6-methylpyrimidin-2-yl)-1H- |
| | pyrrolo[3,2-c]pyridin-6-yl)acetamide |
| | 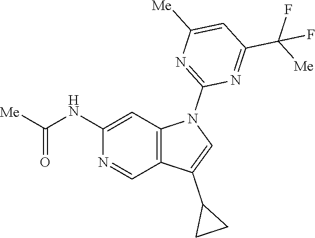 [SMILES: CC(=O)Nc1cc2c(cn1)c(C1CC1)cn2-c1nc(C)cc(C(C)(F)F)n1] |
| | SM-7; RCl: 2-chloro-4-(1,1-difluoroethyl)-6-methylpyrimidine (Preparation |
| | 180) HPLC-C (47-77% MeCN); White solid (62.6 mg, 60%). LCMS m/z = |
| | 372.1 [M + H]⁺; ¹H NMR (500 MHz, CDCl₃) δ: 9.61 (s, 1H), 9.07 (s, 1H), |
| | 8.59 (s, 1H), 7.96 (s, 1H), 7.30 (s, 1H), 2.70 (s, 3H), 2.29 (s, 3H), 2.19 (t, |
| | 3H), 1.97-1.95 (m, 1H), 1.03-1.01 (m, 2H), 0.79-0.77 (m, 2H). |
| 125 | N-(3-cyclopropyl-1-(6-(1,1-difluoroethyl)pyrimidin-4-yl)-1H-pyrrolo[3,2- |
| | c]pyridin-6-yl)acetamide |
| | 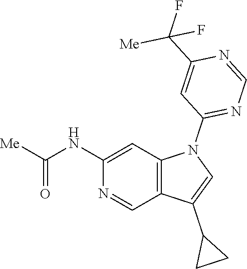 [SMILES: CC(=O)Nc1cc2c(cn1)c(C1CC1)cn2-c1cc(C(C)(F)F)ncn1] |
| | SM-7; 4-chloro-6-(1,1-difluoroethyl)pyrimidine |
| | HPLC-C (Gradient: 38 to 68% MeCN) |
| | 51.7 mg, 62.3% yield as a white solid. LCMS m/z = 358.1 [M + H]⁺ |
| | ¹H NMR (400 MHz, CDCl₃) δ ppm: 9.41 (s, 1H), 9.19 (s, 1H), 8.69 (s, 1H), |
| | 8.64 (s, 1H), 7.65 (s, 1H), 7.43 (s, 1H), 2.28 (s, 3H), 2.01 (t, J = 18.8 Hz, |
| | 3H), 1.98-1.94 (m, 1H), 1.07-1.03 (m, 2H), 0.78-0.77 (m, 2H). |
| 126 | N-(1-(4-(1,1-difluoroethyl)pyrimidin-2-yl)-3-(4-methoxypiperidin-1-yl)- |
| | 1H-pyrazolo[4,3-c]pyridin-6-yl)acetamide |
| | 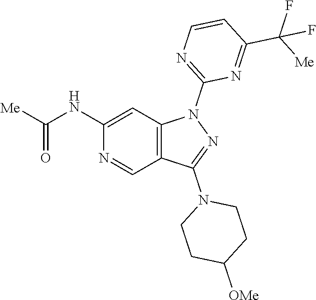 [SMILES: COC1CCN(c2nn(-c3nccc(C(C)(F)F)n3)c3cc(NC(C)=O)ncc23)CC1] |
| | SM-8; RCl: 2-chloro-4-(1,1-difluoroethyl)pyrimidine (Preparation 178) |
| | HPLC-C (25-55% MeCN); Yellow solid (28.2 mg, 29%). LCMS m/z = |
| | 432.1 [M + H]⁺; ¹H NMR (500 MHz, CDCl₃) δ: 9.39 (s, 1H), 8.96 (d, 1H), |
| | 8.79 (s, 1H), 8.16 (br s, 1H), 7.39 (d, 1H), 4.01-3.98 (m, 2H), 3.48-3.47 (m, |
| | 1H), 3.43-3.38 (m, 5H), 2.28 (t, 3H), 2.27 (s, 3H), 2.10-2.09 (m, 2H), 1.82- |
| | 1.81 (m, 2H). |
| 127 | N-(1-(4-(1,1-difluoroethyl)pyrimidin-2-yl)-3-(pyrrolidin-1-yl)-1H- |
| | pyrazolo[4,3-c]pyridin-6-yl)acetamide |
| | 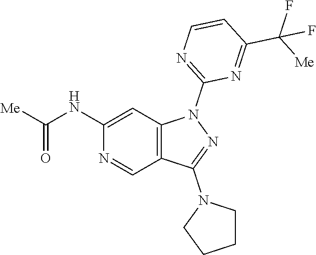 [SMILES: CC(=O)Nc1cc2c(cn1)c(N1CCCC1)nn2-c1nccc(C(C)(F)F)n1] |
| | SM-9; RCl: 2-chloro-4-(1,1-difluoroethyl)pyrimidine (Preparation 178) |
| | HPLC-C (28-58% MeCN); Yellow solid (10.7 mg, 10%). LCMS m/z = |
| | 388.1 [M + H]⁺; ¹H NMR (500 MHz, CDCl₃) δ: 9.51 (s, 1H), 9.42 (br s, 1H), |
| | 8.99 (d, 1H), 8.74 (s, 1H), 7.42 (d, 1H), 3.82-3.79 (m, 4H), 2.32-2.23 (m, |
| | 6H), 2.14-2.12 (m, 4H). |
| 128 | N-(1-(4-(1,1-difluoroethyl)-6-methylpyrimidin-2-yl)-3-ethyl-1H- |
| | pyrazolo[4,3-c]pyridin-6-yl)acetamide |
| | 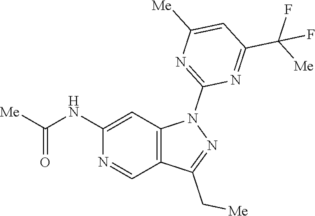 [SMILES: CCc1nn(-c2nc(C)cc(C(C)(F)F)n2)c2cc(NC(C)=O)ncc12] |
| | SM-3; RCl: 2-chloro-4-(1,1-difluoroethyl)-6-methylpyrimidine (Preparation |
| | 180) HPLC-G (35-65% MeCN); White solid (33 mg, 23%); LCMS m/z = |
| | 361.1 [M + H]⁺; ¹H NMR (400 MHz, CDCl₃) δ: 9.41 (s, 1H), 8.75 (s, 1H), |
| | 8.54 (s, 1H), 7.36 (s, 1H), 3.14 (q, 2H), 2.75 (s, 3H), 2.26 (t, 3H), 1.47 (t, |
| | 3H). |
| 129 | N-(1-(4-(1,1-difluoroethyl)-6-methylpyrimidin-2-yl)-3-(4-methylpiperazin- |
| | 1-yl)-1H-pyrazolo[4,3-c]pyridin-6-yl)acetamide |
| | 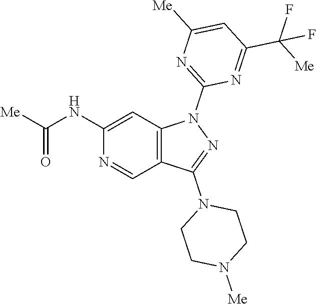 [SMILES: CC(=O)Nc1cc2c(cn1)c(N1CCN(C)CC1)nn2-c1nc(C)cc(C(C)(F)F)n1] |
| | SM-5; RCl: 2-chloro-4-(1,1-difluoroethyl)-6-methylpyrimidine (Preparation |
| | 180) |
| | HPLC-G (36-66% MeCN); White solid (46.4 mg, 49%); LCMS m/z = |
| | 431.1 [M + H]⁺; ¹H NMR (400 MHz, DMSO-d₆) δ: 10.73 (s, 1H), 9.36 (s, |
| | 1H), 9.06 (s, 1H), 7.49 (s, 1H), 3.60 (br s, 4H), 2.64-2.60 (m, 4H), 2.33 (s, |
| | 3H), 2.31 (s, 3H), 2.23-2.13 (m, 6H). |
| 130 | N-(3-(3-cyanoazetidin-1-yl)-1-(4-(1,1-difluoroethyl)-6-methylpyrimidin-2- |
| | yl)-1H-pyrazolo[4,3-c]pyridin-6-yl)acetamide |
| | 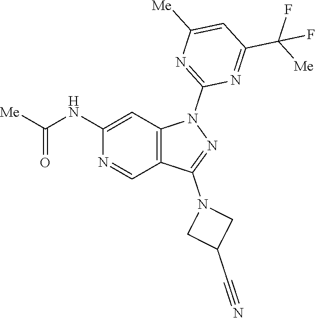 [SMILES: CC(=O)Nc1cc2c(cn1)c(N1CC(C#N)C1)nn2-c1nc(C)cc(C(C)(F)F)n1] |
| | SM-6; RCl: 2-chloro-4-(1,1-difluoroethyl)-6-methylpyrimidine (Preparation |
| | 180) |
| | HPLC-C (30-60% MeCN); Yellow solid (12.5 mg, 19%). LCMS m/z = |
| | 413.1 [M + H]⁺; ¹H NMR (500 MHz, CDCl₃) δ: 9.45 (s, 1H), 8.64 (s, 1H), |
| | 8.54 (s, 1H), 7.33 (s, 1H), 4.68-4.59 (m, 4H), 3.81-3.73 (m, 1H), 2.73 (s, |
| | 3H), 2.29 (s, 3H), 2.24 (t, 3H). |
| 131 | N-(3-cyclopropyl-1-(4-(2-fluoropropan-2-yl)-6-methoxypyrimidin-2-yl)- |
| | 1H-pyrazolo[4,3-c]pyridin-6-yl)acetamide |
| | 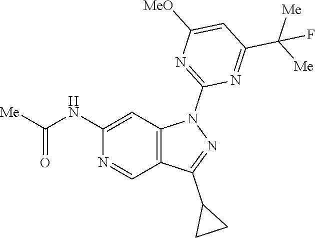 [SMILES: COc1cc(C(C)(C)F)nc(-n2nc(C3CC3)c3cnc(NC(C)=O)cc32)n1] |
| | SM-4; RCl: 2-chloro-4-(2-fluoropropan-2-yl)-6-methoxypyrimidine |
| | (Preparation 223) HPLC-C (41-71% MeCN); White solid (32.6 mg, 37%). |
| | LCMS m/z = 385.1 [M + H]⁺; ¹H NMR (400 MHz, CDCl₃) δ: : 9.46 (s, 1H), |
| | 8.88 (s, 1H), 8.70 (s, 1H), 8.82 (d, 1H), 4.21 (s, 3H), 2.34-2.32 (m, 1H), |
| | 2.28 (s, 3H), 1.85 (s, 3H), 1.80 (s, 3H), 1.28-1.27 (m, 2H), 1.20-1.18 (m, |
| | 2H). |
| 132 | N-(3-cyclopropyl-1-(4-(1,1-difluoroethyl)-6-methoxypyrimidin-2-yl)-1H- |
| | pyrazolo[4,3-c]pyridin-6-yl)acetamide |
| | 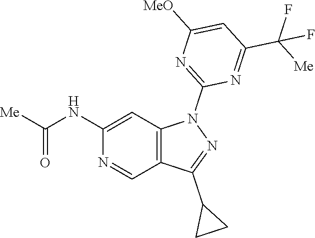 [SMILES: COc1cc(C(C)(F)F)nc(-n2nc(C3CC3)c3cnc(NC(C)=O)cc32)n1] |
| | SM-4; RCl: 2-chloro-4-(1,1-difluoroethyl)-6-methoxypyrimidine |
| | (Preparation 219) |
| | HPLC-C (36-66% MeCN); White solid (23.2 mg, 21%). LCMS m/z = |
| | 389.1 [M + H]⁺; ¹H NMR (400 MHz, CDCl₃) δ: 9.39 (s, 1H), 8.70 (s, 1H), |
| | 8.08 (s, 1H), 6.88 (s, 1H), 4.28 (s, 3H), 2.37-2.374 (m, 1H), 2.25 (s, 3H), |
| | 2.15 (t, J = 18.8 Hz, 3H), 1.30-1.28 (m, 2H), 1.20-1.16 (m, 2H). |
| 133 | N-(3-cyclopropyl-1-(4-(1,1-difluoroethyl)-6-isopropoxypyrimidin-2-yl)- |
| | 1H-pyrazolo[4,3-c]pyridin-6-yl)acetamide |
| | 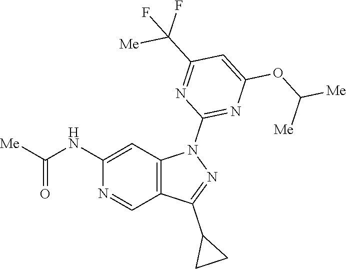 [SMILES: CC(=O)Nc1cc2c(cn1)c(C1CC1)nn2-c1nc(OC(C)C)cc(C(C)(F)F)n1] |
| | SM-4; RCl: 2-chloro-4-(1,1-difluoroethyl)-6-isopropoxypyrimidine |
| | (Preparation 220). HPLC-C (49-79% MeCN); White solid (16.5 mg, 21%). |
| | LCMS m/z = 417.1 [M + H]⁺; ¹H NMR (400 MHz, CDCl₃) δ: 11.57 (br s, |
| | 1H), 9.74 (s, 1H), 8.71 (s, 1H), 6.93 (s, 1H), 5.71-5.65 (m, 1H), 2.39 (s, |
| | 3H), 2.33-2.29 (m, 1H), 2.14 (t, 3H), 1.47 (d, 6H), 1.31-1.29 (m, 4H). |
| 134 | N-(3-cyclopropyl-1-(4-methoxypyrimidin-2-yl)-1H-pyrazolo[4,3-c]pyridin- |
| | 6-yl)acetamide |
| | 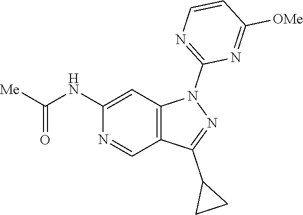 [SMILES: COc1ccnc(-n2nc(C3CC3)c3cnc(NC(C)=O)cc32)n1] |
| | SM-4; RCl: 2-chloro-4-methoxypyrimidine |
| | HPLC-C (22-42% MeCN); White solid (26.9 mg, 22%). LCMS m/z = |
| | 325.1 [M + H]⁺; ¹H NMR (400 MHz, CDCl₃) δ: 9.55 (s, 1H), 9.22 (s, 1H), |
| | 8.71 (s, 1H), 8.52 (d, 1H), 6.63 (d, 1H), 4.31 (s, 3H), 2.38-2.32 (m, 1H), |
| | 2.29 (s, 3H), 1.33-1.30 (m, 2H), 1.24-1.20 (m, 2H). |
| 135 | N-(3-cyclopropyl-1-(4-(2-fluoropropan-2-yl)pyrimidin-2-yl)-1H- |
| | pyrazolo[4,3-c]pyridin-6-yl)acetamide |
| | 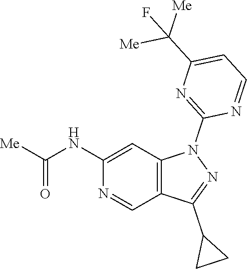 [SMILES: CC(=O)Nc1cc2c(cn1)c(C1CC1)nn2-c1nccc(C(C)(C)F)n1] |
| | SM-4; RCl: 2-chloro-4-(2-fluoropropan-2-yl)pyrimidine (Preparation 222) |
| | HPLC-F (37-67% MeCN); White solid (68.5 mg, 67%); LCMS m/z = 355.1 |
| | [M + H]⁺; ¹H NMR (500 MHz, CDCl₃) δ: 9.37 (s, 1H), 8.86 (d, 1H), 8.72 (s, |
| | 1H), 8.24 (br s, 1H), 7.42 (d, 1H), 2.38-2.36 (m, 1H), 2.27 (s, 3H), 1.93 (s, |
| | 3H), 1.89 (s, 3H), 1.31-1.28 (m, 2H), 1.19-1.16 (m, 2H). |
| 136 | N-(3-(3-cyanoazetidin-1-yl)-1-(4-(1,1-difluoroethyl)-6-methoxypyrimidin- |
| | 2-yl)-1H-pyrazolo[4,3-c]pyridin-6-yl)acetamide |
| | 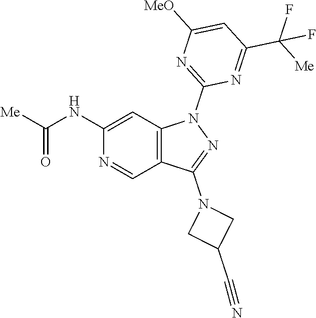 [SMILES: COc1cc(C(C)(F)F)nc(-n2nc(N3CC(C#N)C3)c3cnc(NC(C)=O)cc32)n1] |
| | SM-6; RCl: 2-chloro-4-(1,1-difluoroethyl)-6-methoxypyrimidine |
| | (Preparation 219) |
| | HPLC-F (43-73% MeCN); White solid (21 mg, 49%). LCMS m/z = 429.1 |
| | [M + H]⁺; ¹H NMR (400 MHz, CDCl₃) δ: 9.45 (s, 1H), 8.73 (s, 1H), 8.51 (s, |
| | 1H), 6.86 (s, 1H), 4.66-4.56 (m, 4H), 4.26 (s, 3H), 3.83-3.75 (m, 1H), 2.28 |
| | (s, 3H), 2.13 (d, 3H). |
| 137^{A} | N-(3-cyclopropyl-1-(2-(2-fluoropropan-2-yl)pyrimidin-4-yl)-1H- |
| | pyrazolo[4,3-c]pyridin-6-yl)acetamide |
| | 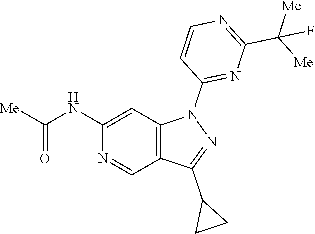 [SMILES: CC(=O)Nc1cc2c(cn1)c(C1CC1)nn2-c1ccnc(C(C)(C)F)n1] |
| | SM-4; RCl: 4-chloro-2-(2-fluoropropan-2-yl)pyrimidine (Preparation 188) |
| | HPLC-C (33-63% MeCN); White solid (31.9 mg, 24%); LCMS m/z = |
| | 355.1 [M + H]⁺; ¹H NMR (500 MHz, MeOH-d₄) δ: 9.44 (s, 1H), 8.91 (s, |
| | 1H), 8.75 (d, 1H), 7.86 (d, 1H), 2.45-2.39 (m, 1H), 2.24 (s, 3H), 1.94 (s, |
| | 3H), 1.90 (s, 3H), 1.24-1.20 (m, 4H). |
| 138 | N-(3-ethyl-1-(2-(2-fluoropropan-2-yl)pyrimidin-4-yl)-1H-pyrazolo[4,3- |
| | c]pyridin-6-yl)acetamide |
| | 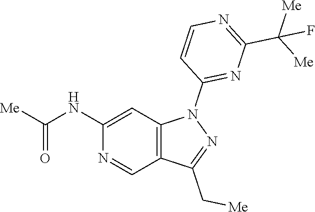 [SMILES: CCc1nn(-c2ccnc(C(C)(C)F)n2)c2cc(NC(C)=O)ncc12] |
| | SM-3; RCl: 4-chloro-2-(2-fluoropropan-2-yl)pyrimidine (Preparation 188) |
| | HPLC-J (42-62% MeCN); White solid (22.7 mg, 14%); LCMS m/z = 343.1 |
| | [M + H]⁺; ¹H NMR (500 MHz, DMSO-d₆) δ: 10.82 (s, 1H), 9.38 (s, 1H), |
| | 8.99 (s, 1H), 8.87 (d, 1H), 7.84 (d, 1H), 3.04 (q , 2H), 2.16 (s, 3H), 1.89 (s, |
| | 3H), 1.85 (s, 3H), 1.39 (t, 3H). |
| 139 | N-(1-(2-(1,1-difluoroethyl)pyrimidin-4-yl)-3-ethyl-1H-pyrazolo[4,3- |
| | c]pyridin-6-yl)acetamide |
| | 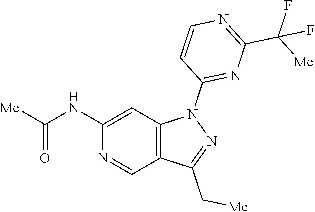 [SMILES: CCc1nn(-c2ccnc(C(C)(F)F)n2)c2cc(NC(C)=O)ncc12] |
| | SM-3; RCl: 4-chloro-2-(1,1-difluoroethyl)pyrimidine (Preparation 184) |
| | SiO2: 50% EtOAc/PE; Yellow solid (25 mg, 26%); LCMS m/z = 347.1 |
| | [M + H]⁺; ¹H NMR (500 MHz, DMSO-d₆) δ: 10.82 (s, 1H), 9.40 (s, 1H), |
| | 9.01 (s, 1H), 8.96 (d, 1H), 8.00 (d, 1H), 3.08 (q, 2H), 2.25 (t, J = 19.5 Hz, |
| | 3H), 2.16 (s, 3H), 1.41 (t, 3H). |
| 140 | N-(3-cyclopropyl-1-(2-(1,1-difluoroethyl)pyrimidin-4-yl)-1H-pyrazolo[4,3- |
| | c]pyridin-6-yl)acetamide |
| | 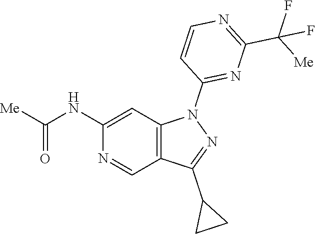 [SMILES: CC(=O)Nc1cc2c(cn1)c(C1CC1)nn2-c1ccnc(C(C)(F)F)n1] |
| | SM-4; RCl: 4-chloro-2-(1,1-difluoroethyl)pyrimidine (Preparation 184) |
| | HPLC-G (42-72% MeCN); White solid (15.3 mg, 13%); LCMS m/z = |
| | 359.1 [M + H]⁺; ¹H NMR (400 MHz, CDCl₃) δ: 9.50 (s, 1H), 8.82 (d, 1H), |
| | 8.76 (s, 1H), 8.09 (s, 1H), 7.90 (d, 1H), 2.35-2.25 (m, 7H), 1.24-1.19 (m, |
| | 4H). |
| 141 | N-(3-cyclopropyl-1-(2-(1,1-difluoroethyl)-6-methoxypyrimidin-4-yl)-1H- |
| | pyrazolo[4,3-c]pyridin-6-yl)acetamide |
| | 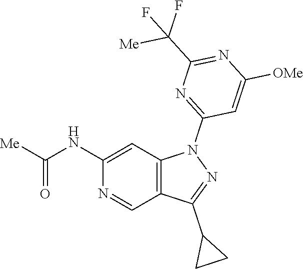 [SMILES: COc1cc(-n2nc(C3CC3)c3cnc(NC(C)=O)cc32)nc(C(C)(F)F)n1] |
| | SM-4; RCl: 4-chloro-2-(1,1-difluoroethyl)-6-methoxypyrimidine |
| | (Preparation 190) |
| | HPLC-F (55-85% MeCN); White solid (50.1 mg, 45%); LCMS m/z = 389.1 |
| | [M + H]⁺; ¹H NMR (500 MHz, CDCl₃) δ: 9.46 (br s, 1H), 8.72 (s, 1H), 8.17 |
| | (s, 1H), 7.22 (s, 1H), 4.09 (s, 3H), 2.30-2.21 (m, 7H), 1.23-1.16 (m, 4H). |
| 142 | N-(3-cyclopropyl-1-(2-(3-fluorooxetan-3-yl)pyrimidin-4-yl)-1H- |
| | pyrazolo[4,3-c]pyridin-6-yl)acetamide |
| | 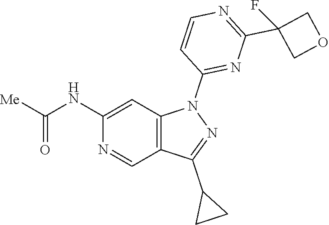 [SMILES: CC(=O)Nc1cc2c(cn1)c(C1CC1)nn2-c1ccnc(C2(F)COC2)n1] |
| | SM-4; RCl: 4-chloro-2-(3-fluorooxetan-3-yl)pyrimidine (Preparation 193) |
| | HPLC-J (38-68% MeCN); White solid (8.2 mg, 8.4%); LCMS m/z = 369.1 |
| | [M + H]⁺; ¹H NMR (400 MHz, CDCl₃) δ: 9.51 (s, 1H), 8.81 (d, 1H), 8.75 (s, |
| | 1H), 8.16 (br s, 1H), 7.85 (d, 1H), 5.39 (d, 1H), 5.34 (d, 1H), 5.25 (d, 1H), |
| | 5.20 (d, 1H), 2.32-2.25 (m, 4H), 1.26-1.19 (m, 4H). |
| 143 | N-(3-cyclopropyl-1-(2-methoxypyrimidin-4-yl)-1H-pyrazolo[4,3-c]pyridin- |
| | 6-yl)acetamide |
| | 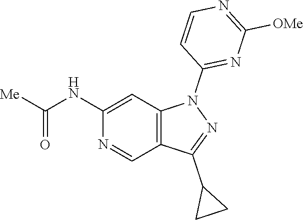 [SMILES: COc1nccc(-n2nc(C3CC3)c3cnc(NC(C)=O)cc32)n1] |
| | SM-4; RCl: 4-chloro-2-methoxypyrimidine |
| | HPLC-C (29-59% MeCN); White solid (3.7 mg, 5%). LCMS m/z = 325.1 |
| | [M + H]⁺; ¹H NMR (500 MHz, CDCl₃) δ: 9.50 (s, 1H), 8.74 (s, 1H), 8.51 (d, |
| | 1H), 8.11 (s, 1H), 7.47 (d, 1H), 4.27 (s, 3H), 2.28-2.27 (m, 1H), 2.26 (s, |
| | 3H), 1.24-1.18 (m, 4H). |
| 144 | N-(3-ethyl-1-(2-(2-methoxypropan-2-yl)pyrimidin-4-yl)-1H-pyrazolo[4,3- |
| | c]pyridin-6-yl)acetamide |
| | 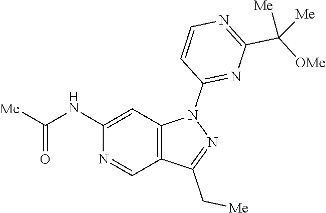 [SMILES: CCc1nn(-c2ccnc(C(C)(C)OC)n2)c2cc(NC(C)=O)ncc12] |
| | SM-3; RCl: 4-chloro-2-(2-methoxypropan-2-yl)pyrimidine (Preparation |
| | 197) |
| | HPLC-G (39-69% MeCN); White solid (43.7 mg, 26%); LCMS m/z = |
| | 355.1 [M + H]⁺; ¹H NMR (500 MHz, CDCl₃) δ: 10.76 (s, 1H), 9.37 (s, 1H), |
| | 8.99 (s, 1H), 8.86 (d, 1H), 7.80 (d, 1H), 3.10 (s, 3H), 3.06 (q, 2H), 2.16 (s, |
| | 3H), 1.69 (s, 6H), 1.40 (t, 3H). |
| 145 | N-(3-cyclopropyl-1-(2-(2-methoxypropan-2-yl)pyrimidin-4-yl)-1H- |
| | pyrazolo[4,3-c]pyridin-6-yl)acetamide |
| | 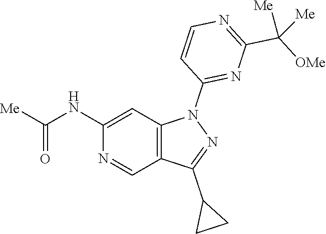 [SMILES: COC(C)(C)c1nccc(-n2nc(C3CC3)c3cnc(NC(C)=O)cc32)n1] |
| | SM-4; RCl: 4-chloro-2-(2-methoxypropan-2-yl)pyrimidine (Preparation |
| | 197) |
| | HPLC-G (38-68% MeCN); White solid (35 mg, 34%); LCMS m/z = 367.2 |
| | [M + H]⁺; ¹H NMR (500 MHz, DMSO-d₆) δ: 10.75 (s, 1H), 9.36 (s, 1H), |
| | 9.00 (s, 1H), 8.84 (d, 1H), 7.75 (d, 1H), 3.09 (s, 3H), 2.52-2.26 (m, 1H), |
| | 2.16 (s, 3H), 1.68 (s, 6H), 1.18-1.16 (m, 4H). |
| 146 | N-(1-(2-(tetrahydrofuran-3-yl)pyrimidin-4-yl)-1H-pyrazolo[4,3-c]pyridin- |
| | 6-yl)acetamide |
| | 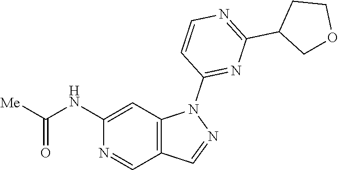 [SMILES: CC(=O)Nc1cc2c(cn1)cnn2-c1ccnc(C2CCOC2)n1] |
| | SM-1; RCl: 4-chloro-2-(tetrahydrofuran-3-yl)pyrimidine (Preparation 200) |
| | HPLC-C (15-45% MeCN); White solid (49 mg, 53%). LCMS m/z = 325.2 |
| | [M + H]⁺; ¹H NMR (400 MHz, MeOH-d₄) δ: 9.48 (s, 1H), 8.88 (d, 1H), 8.74 |
| | (d, 1H), 8.44 (s, 1H), 7.89 (d, 1H), 4.36-4.32 (m, 1H), 4.19-4.15 (m, 2H), |
| | 4.02-4.00 (m, 1H), 3.85-3.84 (m, 1H), 2.70-2.64 (m, 1H), 2.55-2.52 (m, |
| | 1H), 2.24 (s, 3H). |
| 147 | N-(3-cyclopropyl-1-(2-(oxetan-3-yl)pyrimidin-4-yl)-1H-pyrazolo[4,3- |
| | c]pyridin-6-yl)acetamide |
| | 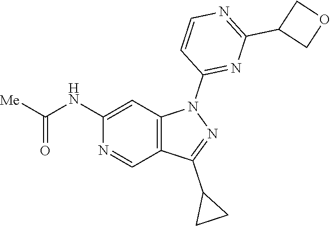 [SMILES: CC(=O)Nc1cc2c(cn1)c(C1CC1)nn2-c1ccnc(C2COC2)n1] |
| | SM-4; RCl: 4-chloro-2-(oxetan-3-yl)pyrimidine (Preparation 201) |
| | HPLC-J (32-62% MeCN); White solid (15 mg, 15%); LCMS m/z = 351.1 |
| | [M + H]⁺; ¹H NMR (400 MHz, CDCl₃) δ: 9.85 (br s, 1H), 9.73 (s, 1H), 8.78- |
| | 8.77 (m, 2H), 7.77 (d, 1H), 5.21-5.19 (m, 4H), 4.70-4.61 (m, 1H), 2.34 (s, |
| | 3H), 2.29-2.25 (m, 1H), 1.27-1.24 (m, 4H). |
| 148 | N-(3-cyclopropyl-1-(2-(2-fluoropropan-2-yl)-6-methoxypyrimidin-4-yl)- |
| | 1H-pyrazolo[4,3-c]pyridin-6-yl)acetamide |
| | 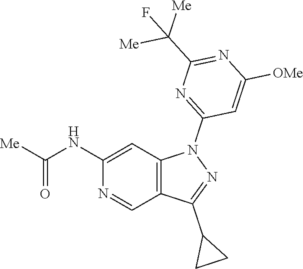 [SMILES: COc1cc(-n2nc(C3CC3)c3cnc(NC(C)=O)cc32)nc(C(C)(C)F)n1] |
| | SM-4; RCl: 4-chloro-2-(2-fluoropropan-2-yl)-6-methoxypyrimidine |
| | (Preparation 208) |
| | HPLC-J (55-85% MeCN); White solid (8 mg, 9%); LCMS m/z = 385.2 |
| | [M + H]⁺; ¹H NMR (400 MHz, CDCl₃) δ: 9.66 (s, 1H), 9.51 (br s, 1H), 8.73 |
| | (s, 1H), 7.14 (s, 1H), 4.08 (s, 3H), 2.32 (s, 3H), 2.27-2.22 (m, 1H), 1.95 (s, |
| | 3H), 1.90 (s, 3H), 1.23-1.20 (m, 4H). |
| 149 | N-(3-cyclopropyl-1-(2-(3-fluorooxetan-3-yl)-6-methoxypyrimidin-4-yl)- |
| | 1H-pyrazolo[4,3-c]pyridin-6-yl)acetamide |
| | 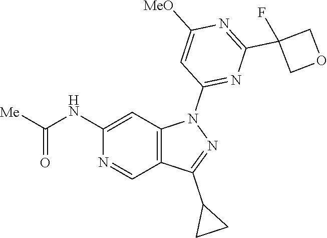 [SMILES: COc1cc(-n2nc(C3CC3)c3cnc(NC(C)=O)cc32)nc(C2(F)COC2)n1] |
| | SM-4; RCl: 4-chloro-2-(3-fluorooxetan-3-yl)-6-methoxypyrimidine |
| | (Preparation 209) |
| | HPLC-J (45-75% MeCN) |
| | White solid (33.8 mg, 19%); LCMS m/z = 399.1 [M + H]⁺; ¹H NMR (400 |
| | MHz, CDCl₃) δ: 9.46 (br s, 1H), 8.71 (s, 1H), 8.16 (s, 1H), 7.18 (s, 1H), |
| | 5.39-5.32 (m, 2H), 5.21-5.14 (m, 2H), 4.08 (s, 3H), 2.30-2.23 (m, 4H), |
| | 1.22-1.15 (m, 4H). |
| 150 | N-(3-cyclopropyl-1-(2-(tetrahydrofuran-3-yl)pyrimidin-4-yl)-1H- |
| | pyrazolo[4,3-c]pyridin-6-yl)acetamide |
| | 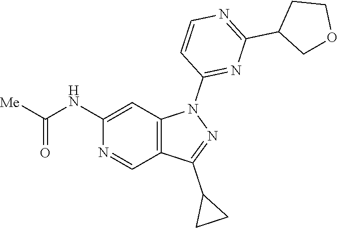 [SMILES: CC(=O)Nc1cc2c(cn1)c(C1CC1)nn2-c1ccnc(C2CCOC2)n1] |
| | SM-4; RCl: 4-chloro-2-(tetrahydrofuran-3-yl)pyrimidine (Preparation 200) |
| | HPLC-C (30-60% MeCN); White solid (29.3 mg, 29%); LCMS m/z = |
| | 365.2 [M + H]⁺; ¹H NMR (500 MHz, CDCl₃) δ: 9.50 (s, 1H), 8.74 (s, 1H), |
| | 8.66 (d, 1H), 8.36 (s, 1H), 7.70 (d, 1H), 4.39-4.35 (m, 1H), 4.21-4.16 (m, |
| | 2H), 4.04-4.02 (m, 1H), 3.87-3.86 (m, 1H), 2.65-2.63 (m, 1H), 2.58-2.56 |
| | (m, 1H), 2.29-2.26 (m, 4H), 1.24-1.18 (m, 4H). |
| 151 | N-(3-cyclopropyl-1-(6-(1,1-difluoroethyl)-2-methylpyrimidin-4-yl)-1H- |
| | pyrazolo[4,3-c]pyridin-6-yl)acetamide |
| | 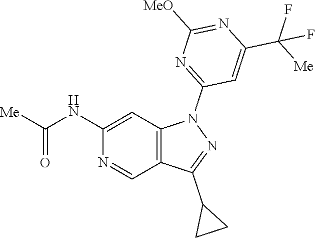 [SMILES: COc1nc(-n2nc(C3CC3)c3cnc(NC(C)=O)cc32)cc(C(C)(F)F)n1] |
| | SM-4; 4-chloro-6-(1,1-difluoroethyl)-2-methylpyrimidine (Preparation 213) |
| | HPLC-C (45-75% MeCN); White solid (25.2 mg, 29%); LCMS m/z = |
| | 373.1 [M + H]⁺; ¹H NMR (500 MHz, MeOH-d₄) δ: 9.43 (s, 1H), 8.83 (s, |
| | 1H), 7.83 (s, 1H), 2.80 (s, 3H), 2.34-2.43 (m, 1H), 2.25 (s, 3H), 2.00 (t, |
| | 3H), 1.26-1.13 (m, 4H). |
| 152 | N-(3-(3-cyanoazetidin-1-yl)-1-(2-(1,1-difluoroethyl)-6-methoxypyrimidin- |
| | 4-yl)-1H-pyrazolo[4,3-c]pyridin-6-yl)acetamide |
| | 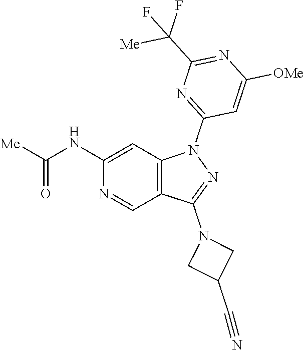 [SMILES: COc1cc(-n2nc(N3CC(C#N)C3)c3cnc(NC(C)=O)cc32)nc(C(C)(F)F)n1] |
| | SM-6; RCl: 4-chloro-2-(1, 1-difluoroethyl)-6-methoxypyrimidine |
| | (Preparation 190) |
| | HPLC-C (37-67% MeCN); White solid (3.1 mg, 4%); LCMS m/z = 429.1 |
| | [M + H]⁺; ¹H NMR (500 MHz, CDCl₃) δ: 9.58 (s, 1H), 9.08 (br s, 1H), 8.49 |
| | (s, 1H), 7.12 (s, 1H), 4.61-4.57 (m, 2H), 4.55-4.52 (m, 2H), 4.10 (s, 3H), |
| | 3.84-3.79 (m, 1H), 2.30 (s, 3H), 2.23 (t, 3H). |
| 153 | N-(1-(2-(1,1-difluoroethyl)-6-methoxypyrimidin-4-yl)-3-(4- |
| | methylpiperazin-1-yl)-1H-pyrazolo[4,3-c]pyridin-6-yl)acetamide |
| | 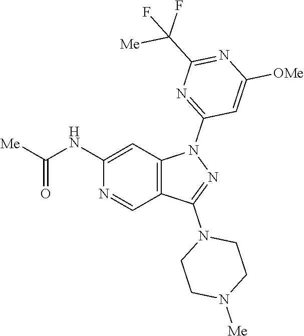 [SMILES: COc1cc(-n2nc(N3CCN(C)CC3)c3cnc(NC(C)=O)cc32)nc(C(C)(F)F)n1] |
| | SM-5; RCl: 4-chloro-2-(1,1-difluoroethyl)-6-methoxypyrimidine |
| | (Preparation 190) |
| | HPLC-J (45-75% MeCN); White solid (23.4 mg, 18%); LCMS m/z = 447.2 |
| | [M + H]⁺; ¹H NMR (500 MHz, CDCl₃) δ: 9.48 (br s, 1H), 8.73 (s, 1H), 8.05 |
| | (s, 1H), 7.12 (s, 1H), 4.08 (s, 3H), 3.67-3.65 (m, 4H), 2.66-2.65 (m, 4H), |
| | 2.40 (s, 3H), 2.29-2.21 (m, 6H). |
| 154 | N-(3-(3-cyanoazetidin-1-yl)-1-(2-(1,1-difluoroethyl)-6-methylpyrimidin-4- |
| | yl)-1H-pyrazolo[4,3-c]pyridin-6-yl)acetamide |
| | 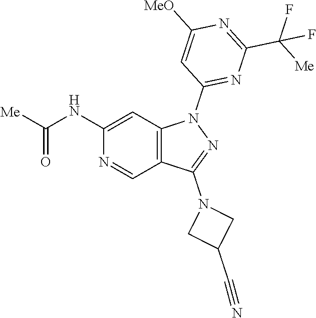 [SMILES: COc1cc(-n2nc(N3CC(C#N)C3)c3cnc(NC(C)=O)cc32)nc(C(C)(F)F)n1] |
| | SM-6; RCl: 4-chloro-2-(1,1-difluoroethyl)-6-methylpyrimidine (Preparation |
| | 218) |
| | HPLC-C (28-58% MeCN); White solid (28 mg, 39%); LCMS m/z = 413.1 |
| | [M + H]⁺; ¹H NMR (500 MHz, DMSO-d₆) δ: 10.77 (s, 1H), 9.34 (s, 1H), |
| | 8.79 (d, 1H), 7.66 (s, 1H), 4.61-4.54 (m, 2H), 4.50-4.46 (m, 2H), 4.09-4.03 |
| | (m, 1H), 2.58 (s, 3H), 2.21 (t, 3H), 2.15 (s, 3H). |
| 155 | N-(3-cyclopropyl-1-(2-(1,1-difluoroethyl)-6-methylpyrimidin-4-yl)-1H- |
| | pyrazolo[4,3-c]pyridin-6-yl)acetamide |
| | 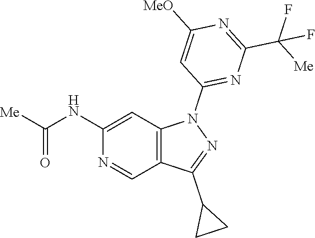 [SMILES: COc1cc(-n2nc(C3CC3)c3cnc(NC(C)=O)cc32)nc(C(C)(F)F)n1] |
| | SM-4; RCl: 4-chloro-2-(1,1-difluoroethyl)-6-methylpyrimidine (Preparation |
| | 218) |
| | SiO₂: 0-5% MeOH/DCM; Yellow solid (39 mg, 45%); LCMS m/z = 373.2 |
| | [M + H]⁺; ¹H NMR (500 MHz, MeOH-d₄) δ: 9.37 (s, 1H), 8.86 (s, 1H), 7.82 |
| | (s, 1H), 2.64 (s, 3H), 2.45-2.35 (m, 1H), 2.27-2.19 (m, 6H), 1.24-1.18 (m, |
| | 4H). |
| 156 | N-(3-cyclopropyl-1-(2-(3-fluorotetrahydrofuran-3-yl)pyrimidin-4-yl)-1H- |
| | pyrazolo[4,3-c]pyridin-6-yl)acetamide |
| | 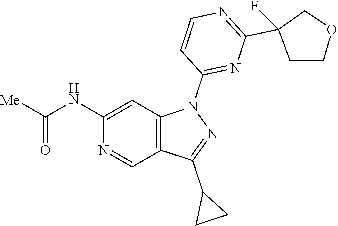 [SMILES: CC(=O)Nc1cc2c(cn1)c(C1CC1)nn2-c1ccnc(C2(F)CCOC2)n1] |
| | SM-4; RCl: 4-chloro-2-(3-fluorotetrahydrofuran-3-yl)pyrimidine |
| | (Preparation 194) |
| | HPLC-C (31-61% MeCN); White solid (61.7 mg, 58%); LCMS m/z = |
| | 383.2 [M + H]⁺; ¹H NMR (400 MHz, CDCl₃) δ: 9.56 (s, 1H), 8.92 (s, 1H), |
| | 8.79 (d, 1H), 8.75 (s, 1H), 7.82 (d, 1H), 4.49-4.31 (m, 4H), 3.05-2.93 (m, |
| | 1H), 2.80-2.70 (m, 1H), 2.30 (s, 3H), 2.28-2.22 (m, 1H), 1.25-1.22 (m, 4H). |
| 157 | N-(3-cyclopropyl-1-(6-methoxy-2-(oxetan-3-yl)pyrimidin-4-yl)-1H- |
| | pyrazolo[4,3-c]pyridin-6-yl)acetamide |
| | 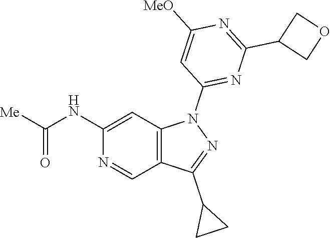 [SMILES: COc1cc(-n2nc(C3CC3)c3cnc(NC(C)=O)cc32)nc(C2COC2)n1] |
| | SM-4; RCl: 4-chloro-6-methoxy-2-(oxetan-3-yl)pyrimidine (Preparation |
| | 373) |
| | HPLC-C (38-68% MeCN); White solid (13.6 mg, 11%); LCMS m/z = |
| | 381.1 [M + H]⁺; ¹H NMR (500 MHz, CDCl₃) δ: 9.60 (s, 1H), 9.10 (br s, 1H), |
| | 8.71 (s, 1H), 7.10 (s, 1H), 5.24-5.11 (m, 4H), 4.54 (m, 1H), 4.06 (s, 3H), |
| | 2.31 (s, 3H), 2.29-2.23 (m, 1H), 1.24-1.19 (m, 4H). |
| 158 | N-(3-cyclopropyl-1-(2-(3-fluorotetrahydrofuran-3-yl)-6-methoxypyrimidin- |
| | 4-yl)-1H-pyrazolo[4,3-c]pyridin-6-yl)acetamide |
| | 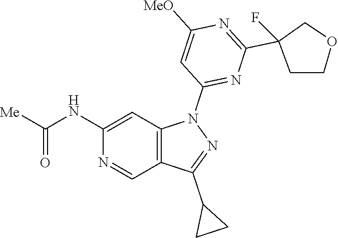 [SMILES: COc1cc(-n2nc(C3CC3)c3cnc(NC(C)=O)cc32)nc(C2(F)CCOC2)n1] |
| | SM-4; RCl: 4-chloro-2-(3-fluorotetrahydrofuran-3-yl)-6- |
| | methoxypyrimidine (Preparation 210) |
| | HPLC-C (44-74% MeCN); White solid (31.8 mg, 28%). LCMS m/z = |
| | 413.1 [M + H]⁺; ¹H NMR (500 MHz, CDCl₃) δ: 9.44 (s, 1H), 8.72 (s, 1H), |
| | 8.19 (s, 1H), 7.15 (s, 1H), 4.47-4.28 (m, 4H), 4.06 (s, 3H), 3.00-2.95 (m, |
| | 1H), 2.94-2.89 (m, 1H), 2.29-2.25 (m, 4H), 1.23-1.17 (m, 4H). |

A-K₂CO₃ was used instead of Cs₂CO₃

### Example 159 and 160: N-(1-(4-(1,1-difluoroethyl)-6-methoxypyrimidin-2-yl)-3-(4-methylpiperazin-1-yl)-1H-pyrazolo[4,3-c]pyridin-6-yl)acetamide and N-(1-(6-(1,1-difluoroethyl)-2-methoxypyrimidin-4-yl)-3-(4-methylpiperazin-1-yl)-1H-pyrazolo[4,3-c]pyridin-6-yl)acetamide

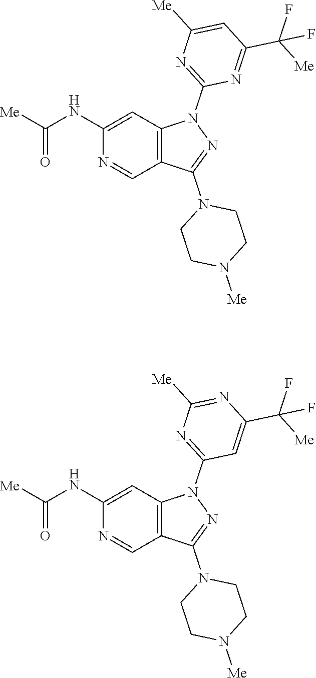 [SMILES: CC(=O)Nc1cc2c(cn1)c(N1CCN(C)CC1)nn2-c1nc(C)cc(C(C)(F)F)n1 ; CC(=O)Nc1cc2c(cn1)c(N1CCN(C)CC1)nn2-c1cc(C(C)(F)F)nc(C)n1]

To a solution of 2-chloro-4-(1,1-difluoroethyl)-6-methoxypyrimidine and 4-chloro-6-(1,1-difluoroethyl)-2-methoxypyrimidine (Preparation 219, 60 mg, 0.288 mmol) in DMF (3 mL) was added compound 5 (78.91 mg, 0.288 mmol) and Cs₂CO₃ (187.44 mg, 0.575 mmol), and the resulting mixture was stirred at 80° C. for 6 h. The cooled mixture was concentrated and was purified by HPLC-F (33-63% MeCN) to give N-(1-(4-(1,1-difluoroethyl)-6-methoxypyrimidin-2-yl)-3-(4-methylpiperazin-1-yl)-1H-pyrazolo[4,3-c]pyridin-6-yl)acetamide (16.5 mg, 12.9% yield) as a yellow solid. LCMS m/z=447.2 [M+H]⁺; ¹H NMR (400 MHz, CDCl₃) δ: 9.41 (s, 1H), 8.75 (s, 1H), 8.08 (s, 1H), 6.82 (s, 1H), 4.26 (s, 3H), 3.80 (br s, 4H), 2.79 (br s, 4H), 2.48 (s, 3H), 2.22 (s, 3H), 2.14 (t, 3H) and N-(1-(6-(1,1-difluoroethyl)-2-methoxypyrimidin-4-yl)-3-(4-methylpiperazin-1-yl)-1H-pyrazolo[4,3-c]pyridin-6-yl)acetamide (5.2 mg, 4.05% yield) as a yellow solid. LCMS m/z=447.2 [M+H]⁺; ¹H NMR (400 MHz, CDCl₃) δ: 9.41 (s, 1H), 8.75 (s, 1H), 8.08 (s, 1H), 6.82 (s, 1H), 4.26 (s, 3H), 3.80 (br s, 4H), 2.79 (br s, 4H), 2.48 (s, 3H), 2.22 (s, 3H), 2.14 (t, 3H).

### Example 161 and 162: N-(3-cyclopropyl-1-(4-(1,1-difluoroethyl)-6-(2-methoxyethoxy)pyrimidin-2-yl)-1H-pyrazolo[4,3-c]pyridin-6-yl)acetamide and N-(3-cyclopropyl-1-(6-(1,1-difluoroethyl)-2-(2-methoxyethoxy)pyrimidin-4-yl)-1H-pyrazolo[4,3-c]pyridin-6-yl)acetamide

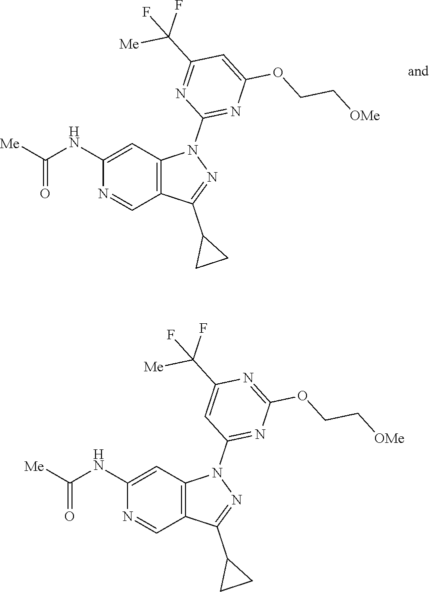 [SMILES: COCCOc1cc(C(C)(F)F)nc(-n2nc(C3CC3)c3cnc(NC(C)=O)cc32)n1 ; COCCOc1nc(-n2nc(C3CC3)c3cnc(NC(C)=O)cc32)cc(C(C)(F)F)n1]

To a solution of N-(3-cyclopropyl-1H-pyrazolo[4,3-c]pyridin-6-yl)acetamide (Preparation 11, 60 mg, 0.277 mmol) in DMF (3 mL) was added a mixture of 2-chloro-4-(1,1-difluoroethyl)-6-(2-methoxyethoxy)pyrimidine and 4-chloro-6-(1,1-difluoroethyl)-2-(2-methoxyethoxy)pyrimidine (Preparation 224, 70.1 mg, 0.277 mmol) and Cs₂CO₃ (181 mg, 0.555 mmol) and the resulting mixture was stirred at 70° C. for 12 h. The mixture was concentrated and purified by prep-HPLC-C (33-63% MeCN) to give the title compounds.

Peak 1, N-(3-cyclopropyl-1-(4-(1,1-difluoroethyl)-6-(2-methoxyethoxy)pyrimidin-2-yl)-1H-pyrazolo[4,3-c]pyridin-6-yl)acetamide (white solid, 19.9 mg, 16%). LCMS m/z=433.1 [M+H]⁺; ¹H NMR (400 MHz, CDCl₃) δ: 9.36 (s, 1H), 8.70 (s, 1H), 8.08 (s, 1H), 6.95 (s, 1H), 4.85-4.86 (m, 2H), 3.88-3.86 (m, 2H), 3.47 (s, 3H), 2.37-2.33 (m, 1H), 2.25 (s, 3H), 2.15 (t, J=18.8 Hz, 3H), 1.29-1.26 (m, 2H), 1.18-1.16 (m, 2H).

Peak 2, N-(3-cyclopropyl-1-(6-(1,1-difluoroethyl)-2-(2-methoxyethoxy)pyrimidin-4-yl)-1H-pyrazolo[4,3-c]pyridin-6-yl)acetamide (white solid, 11.7 mg, 10%). LCMS m/z=433.1 [M+H]⁺; ¹H NMR (400 MHz, CDCl₃) δ: 9.44 (s, 1H), 8.74 (s, 1H), 8.08 (s, 1H), 7.76 (s, 1H), 4.86-4.84 (m, 2H), 3.95-3.93 (m, 2H), 3.48 (s, 3H), 2.28-2.27 (m, 1H), 2.25 (s, 3H), 2.00 (t, J=18.8 Hz, 3H), 1.26-1.19 (m, 4H).

### Example 163 and 164: N-(1-(4-chloro-6-(2-fluoropropan-2-yl)pyrimidin-2-yl)-3-cyclopropyl-1H-pyrazolo[4,3-c]pyridin-6-yl)acetamide and N-(1-(2-chloro-6-(2-fluoropropan-2-yl)pyrimidin-4-yl)-3-cyclopropyl-1H-pyrazolo[4,3-c]pyridin-6-yl)acetamide

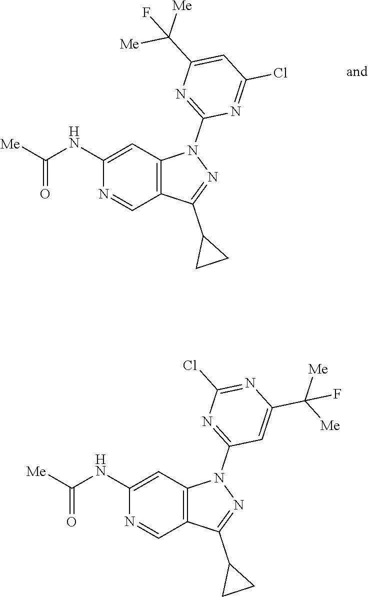 [SMILES: CC(=O)Nc1cc2c(cn1)c(C1CC1)nn2-c1nc(Cl)cc(C(C)(C)F)n1 ; CC(=O)Nc1cc2c(cn1)c(C1CC1)nn2-c1cc(C(C)(C)F)nc(Cl)n1]

To a solution of N-(3-cyclopropyl-1H-pyrazolo[4,3-c]pyridin-6-yl)acetamide (Preparation 11, 100 mg, 0.462 mmol) in DMSO (3 mL) was added 2,4-dichloro-6-(2-fluoropropan-2-yl)pyrimidine (Preparation 221, 96.7 mg, 0.462 mmol) and DBU (70.4 mg, 0.462 mol) and the resulting mixture stirred at 80° C. for 2 h. The mixture was purified by HPLC-C (47-77% MeCN) to give the title compounds.

Peak 1, N-(1-(4-chloro-6-(2-fluoropropan-2-yl)pyrimidin-2-yl)-3-cyclopropyl-1H-pyrazolo[4,3-c]pyridin-6-yl)acetamide (yellow solid, 15.2 mg, 8.5%). LCMS m/z=389.1 [M+H]⁺. ¹H NMR (400 MHz, CDCl₃) δ: 9.42 (s, 1H), 8.79 (s, 1H), 8.70 (s, 1H), 7.45 (d, 1H), 2.39-2.36 (m, 1H), 2.30 (s, 3H), 1.92 (s, 3H), 1.87 (s, 3H), 1.31-1.28 (m, 2H), 1.22-1.19 (m, 2H).

Peak 2, N-(1-(2-chloro-6-(2-fluoropropan-2-yl)pyrimidin-4-yl)-3-cyclopropyl-1H-pyrazolo[4,3-c]pyridin-6-yl)acetamide (white solid, 44.9 mg, 25%). LCMS m/z=389.1 [M+H]⁺. ¹H NMR (400 MHz, CDCl₃) δ: 9.51 (s, 1H), 8.72 (s, 1H), 8.68 (s, 1H), 7.94 (s, 1H), 2.28-2.21 (m, 4H), 1.68 (s, 3H), 1.74 (s, 3H), 1.25-1.18 (m, 4H).

### Example 165: N-(1-(3-(1,1-difluoroethyl)pyrazin-2-yl)-3-methyl-1H-pyrazolo[4,3-c]pyridin-6-yl)acetamide

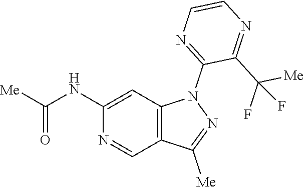 [SMILES: CC(=O)Nc1cc2c(cn1)c(C)nn2-c1nccnc1C(C)(F)F]

To a solution of N-(3-methyl-1H-pyrazolo[4,3-c]pyridin-6-yl)acetamide (Preparation 6, 100 mg, 0.560 mmol), 2-chloro-3-(1,1-difluoroethyl)pyrazine (Preparation 173, 106.5 mg, 0.560 mmol) in dioxane (5 mL) was added XantPhos (64.8 mg, 0.112 mmol), Pd₂(dba)₃ (51.3 mg, 0.056 mmol) and Zn(OAc)₂ (51.4 mg, 0.280 mmol) and the reaction mixture stirred at 120° C. for 12 h under N₂. The mixture was evaporated to dryness and the residue was purified by HPLC-J (33-63% MeCN) to give N-(1-(3-(1,1-difluoroethyl)pyrazin-2-yl)-3-methyl-1H-pyrazolo[4,3-c]pyridin-6-yl)acetamide as a white solid (6.0 mg, 3.2%). LCMS m/z=333.0 [M+H]⁺; ¹H NMR (400 MHz, CDCl₃) δ: 8.72 (s, 1H), 8.70 (d, 1H), 8.65 (d, 1H), 8.48 (s, 1H), 8.09 (br s, 1H), 2.65 (s, 3H), 2.25 (t, 1H), 2.22 (s, 3H).

### Example 166: N-(3-cyclopropyl-1-(2-(1,1-difluoroethyl)pyrimidin-4-yl)-1H-pyrrolo[3,2-c]pyridin-6-yl)acetamide

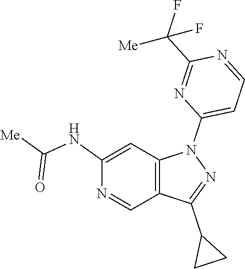 [SMILES: CC(=O)Nc1cc2c(cn1)c(C1CC1)nn2-c1ccnc(C(C)(F)F)n1]

A mixture of 6-chloro-3-cyclopropyl-1-(2-(1,1-difluoroethyl)pyrimidin-4-yl)-1H-pyrrolo[3,2-c]pyridine (Preparation 350, 150 mg, 0.448 mmol), acetamide (132.3 mg, 2.24 mmol), Cs₂CO₃ (292 mg, 0.896 mmol) and BrettPhos Pd G3 (20.31 mg, 0.022 mmol) in dioxane (4 mL) was purged with N₂, then heated at 110° C. in a sealed tube for 1 h. The cooled mixture was filtered, the filtrate was concentrated in vacuo and the crude was purified by chromatography on silica gel (0-80% EtOAc-EtOH 3:1 with 2% NH₄OH in heptane) to give N-(3-cyclopropyl-1-(2-(1,1-difluoroethyl)pyrimidin-4-yl)-1H-pyrrolo[3,2-c]pyridin-6-yl)acetamide (131 mg, 81% yield) as a white powder. LCMS m/z=358.1 [M+H]⁺1H NMR (400 MHz, CDCl₃) δ 9.28 (s, 1H), 8.85 (d, 1H, J=5.8 Hz), 8.66 (d, 1H, J=0.8 Hz), 8.26 (br s, 1H), 7.50-7.40 (m, 2H), 2.30-2.20 (m, 6H), 2.00-1.90 (m, 1H), 1.10-1.00 (m, 2H), 0.80-0.70 (m, 2H).

### Example 167: N-(3-cyclopropyl-1-(6-(3-fluorooxetan-3-yl)pyrazin-2-yl)-1H-pyrazolo[4,3-c]pyridin-6-yl)acetamide

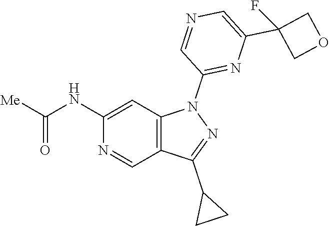 [SMILES: CC(=O)Nc1cc2c(cn1)c(C1CC1)nn2-c1cncc(C2(F)COC2)n1]

To a solution of 2-bromo-6-(3-fluorooxetan-3-yl)pyrazine (Preparation 369, 100 mg, 0.429 mmol) and N-(3-cyclopropyl-1H-pyrazolo[4,3-c]pyridin-6-yl)acetamide (Preparation 11, 92.8 mg, 0.429 mmol) in dioxane (5 mL) was added N,N′-dimethylethane-1,2-diamine (7.6 mg, 0.086 mmol), K₂CO₃ (118.6 mg, 0.858 mmol) and CuI (32.7 mg, 0.172 mmol) and the reaction mixture was stirred at 100° C. for 2 h under N₂. The cooled mixture was concentrated and purified by prep-TLC (PE/EtOAc=1/2) to give N-(3-cyclopropyl-1-(6-(3-fluorooxetan-3-yl)pyrazin-2-yl)-1H-pyrazolo[4,3-c]pyridin-6-yl)acetamide (28.0 mg, 17.7% yield) as a white solid. LCMS m/z=369.1 [M+H]⁺. ¹H NMR (400 MHz, CDCl₃) δ: 9.39 (s, 1H), 9.30 (s, 1H), 8.74 (s, 1H), 8.64 (s, 1H), 8.51 (br s, 1H), 5.43 (d, 1H), 5.37 (d, 1H), 5.19 (d, 1H), 5.13 (d, 1H), 2.33-2.30 (m, 1H), 2.27 (s, 3H), 1.27-1.20 (m, 4H).

### Example 168: N-(1-(6-(3-methoxytetrahydrofuran-3-yl)pyrimidin-4-yl)-1H-pyrazolo[4,3-c]pyridin-6-yl)acetamide

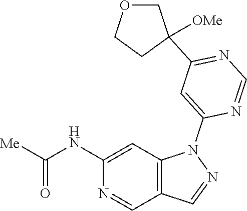 [SMILES: COC1(c2cc(-n3ncc4cnc(NC(C)=O)cc43)ncn2)CCOC1]

N-(1-(6-(3-Methoxytetrahydrofuran-3-yl)pyrimidin-4-yl)-1H-pyrazolo[4,3-c]pyridin-6-yl)acetamide was obtained as a white solid, 13 mg, 10% yield from N-(1H-pyrazolo[4,3-c]pyridin-6-yl)acetamide (Preparation 2) and 4-bromo-6-(3-methoxytetrahydrofuran-3-yl)pyrimidine (Preparation 167), following a similar procedure to that described in Example 167. The crude product was purified by HPLC-G (15-45% MeCN). LCMS m/z=355.2 [M+H]⁺. ¹H NMR (400 MHz, MeOH-d₄) δ: 9.48 (s, 1H), 9.12 (s, 1H), 8.90 (s, 1H), 8.48 (s, 1H), 8.21 (s, 1H), 4.17-4.06 (m, 4H), 3.34 (s, 3H), 2.67-2.62 (m, 1H), 2.50-2.45 (m, 1H), 2.25 (s, 3H).

### Example 169: N-(1-(4-(1,1-difluoroethyl)pyrimidin-2-yl)-3-(3,4-dimethylpiperazin-1-yl)-1H-pyrazolo[4,3-c]pyridin-6-yl)acetamide

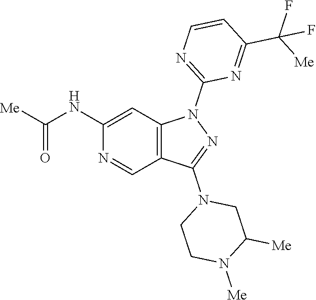 [SMILES: CC(=O)Nc1cc2c(cn1)c(N1CCN(C)C(C)C1)nn2-c1nccc(C(C)(F)F)n1]

To a solution of 6-chloro-1-(4-(1,1-difluoroethyl)pyrimidin-2-yl)-3-(3,4-dimethylpiperazin-1-yl)-1H-pyrazolo[4,3-c]pyridine (Preparation 293, 108 mg, 0.265 mmol) in dioxane (2 mL) was added acetamide (18.8 mg, 0.318 mmol), Cs₂CO₃ (172.56 mg, 0.53 mmol) and Brettphos Pd G3 (24 mg, 0.026 mmol). The resulting mixture was stirred at 100° C. for 5 h under N₂. The reaction mixture was concentrated and the residue purified by prep-HPLC-F (36-66% MeCN) to give N-(1-(4-(1,1-difluoroethyl)pyrimidin-2-yl)-3-(3,4-dimethylpiperazin-1-yl)-1H-pyrazolo[4,3-c]pyridin-6-yl)acetamide as a yellow solid (18.7 mg, 16.4%). LCMS m/z=431.1 [M+H]⁺ 1H NMR: (400 MHz, DMSO-d₆) δ ppm: 10.70 (s, 1H), 9.28 (s, 1H), 9.04-9.01 (m, 2H), 7.54 (d, J=4.8 Hz, 1H), 3.98-3.87 (m, 2H), 3.13-3.12 (m, 1H), 2.85-2.73 (m, 2H), 2.33-2.32 (m, 1H), 2.24-2.19 (m, 6H), 2.14-2.12 (m, 4H), 1.11-1.04 (m, 3H).

Example 170-228: The title compounds were prepared from acetamide and the appropriate chloride (SM) using an analogous method to that described for Example 169.

| Example | |
|---|---|
| No | Name/Structure/SM/Data |
| 170 | N-(1-(2-(1,1-difluoroethyl)pyrimidin-4-yl)-3-(3,4-dimethylpiperazin-1-yl)- |
| | 1H-pyrazolo[4,3-c]pyridin-6-yl)acetamide |
| | 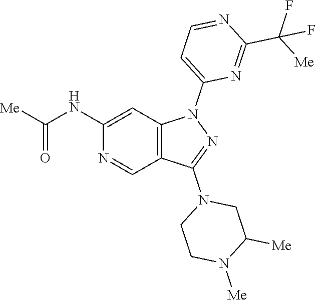 [SMILES: CC(=O)Nc1cc2c(cn1)c(N1CCN(C)C(C)C1)nn2-c1ccnc(C(C)(F)F)n1] |
| | SM: 6-chloro-1-(2-(1,1-difluoroethyl)pyrimidin-4-yl)-3-(3,4- |
| | dimethylpiperazin-1-yl)-1H-pyrazolo[4,3-c]pyridine (Preparation 294). |
| | HPLC-C (28-58% MeCN) |
| | White solid (15 mg, 16%). LCMS m/z = 431.2 [M + H]⁺. ¹H NMR (500 |
| | MHz, DMSO-d₆) δ: 10.78 (s, 1H), 9.41 (s, 1H), 9.12 (s, 1H), 8.87 (d, 1H), |
| | 7.85 (d, 1H), 4.07-3.99 (m, 2H), 3.46-3.41 (m, 1H), 3.31-3.27 (m, 1H), |
| | 2.90-2.89 (m, 2H), 2.30-2.19 (m, 7H), 2.17 (s, 3H), 1.16-1.14 (m, 3H). |
| 171 | N-(1-(4-(1,1-difluoroethyl)pyrimidin-2-yl)-3-(4-methyl-3-oxopiperazin-1- |
| | yl)-1H-pyrazolo[4,3-c]pyridin-6-yl)acetamide |
| | 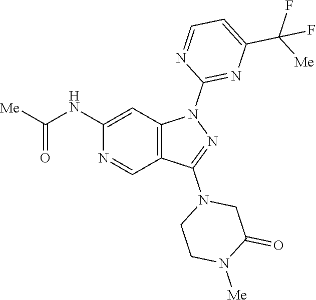 [SMILES: CC(=O)Nc1cc2c(cn1)c(N1CCN(C)C(=O)C1)nn2-c1nccc(C(C)(F)F)n1] |
| | SM: 4-(6-chloro-1-(4-(1,1-difluoroethyl)pyrimidin-2-yl)-1H-pyrazolo[4,3- |
| | c]pyridin-3-yl)-1-methylpiperazin-2-one (Preparation 295) |
| | HPLC-F (29-59% MeCN) |
| | White solid (10.1 mg, 13%). LCMS m/z = 431.1 [M + H]⁺. ¹H NMR (400 |
| | MHz, CDCl₃) δ: 9.41 (s, 1H), 8.97 (d, 1H), 8.82 (s, 1H), 8.12 (s, 1H), 7.39 |
| | (d, 1H), 4.40 (s, 2H), 4.00-3.97 (m, 2H), 3.62-3.59 (m, 2H), 3.09 (s, 3H), |
| | 2.33-2.23 (m, 6H). |
| 172 | N-(1-(2-(1,1-difluoroethyl)pyrimidin-4-yl)-3-(4-methyl-3-oxopiperazin-1- |
| | yl)-1H-pyrazolo[4,3-c]pyridin-6-yl)acetamide |
| | 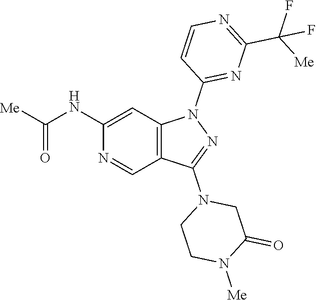 [SMILES: CC(=O)Nc1cc2c(cn1)c(N1CCN(C)C(=O)C1)nn2-c1ccnc(C(C)(F)F)n1] |
| | SM: 4-(6-chloro-1-(2-(1,1-difluoroethyl)pyrimidin-4-yl)-1H-pyrazolo[4,3- |
| | c]pyridin-3-yl)-1-methylpiperazin-2-one (Preparation 296) |
| | HPLC-F (30-60% MeCN) |
| | White solid (23.4 mg, 11%). LCMS m/z = 431.2 [M + H]⁺. ¹H NMR (500 |
| | MHz, CDCl₃) δ: 9.54 (s, 1H), 8.79-8.77 (m, 2H), 8.22 (s, 1H), 7.78 (d, 1H), |
| | 4.35 (s, 2H), 3.94-3.92 (m, 2H), 3.62-3.60 (m, 2H), 3.09 (s, 3H), 2.32-2.25 |
| | (m, 6H). |
| 173 | N-(1-(4-(1,1-difluoroethyl)pyrimidin-2-yl)-3-(3-methoxypiperidin-1-yl)- |
| | 1H-pyrazolo[4,3-c]pyridin-6-yl)acetamide |
| | 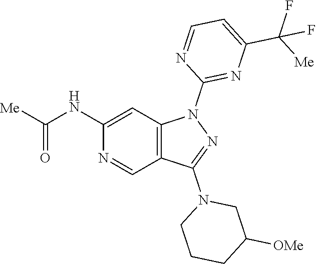 [SMILES: COC1CCCN(c2nn(-c3nccc(C(C)(F)F)n3)c3cc(NC(C)=O)ncc23)C1] |
| | SM: 6-chloro-1-(4-(1,1-difluoroethyl)pyrimidin-2-yl)-3-(3- |
| | methoxypiperidin-1-yl)-1H-pyrazolo[4,3-c]pyridine (Preparation 297) |
| | HPLC-F (35-65% MeCN) |
| | LCMS m/z = 432.1 [M + H]⁺; ¹H NMR (400 MHz, CDCl₃) δ: 9.41 (s, 1H), |
| | 8.97 (d, 1H), 8.81 (s, 1H), 8.32 (s, 1H), 7.40 (d, 1H), 3.99-3.95 (m, 1H), |
| | 3.65-3.59 (m, 1H), 3.54-3.53 (m, 1H), 3.46 (s, 3H), 3.43-3.38 (m, 2H), |
| | 2.33-2.23 (m, 6H), 2.04-1.99 (m, 2H), 1.76-1.71 (m, 2H). |
| 174 | N-(1-(2-(1,1-difluoroethyl)pyrimidin-4-yl)-3-(3-methoxypiperidin-1-yl)- |
| | 1H-pyrazolo[4,3-c]pyridin-6-yl)acetamide |
| | 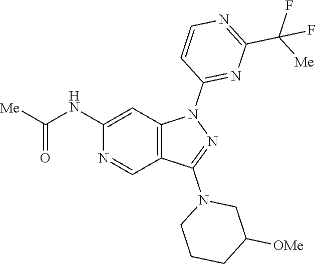 [SMILES: COC1CCCN(c2nn(-c3ccnc(C(C)(F)F)n3)c3cc(NC(C)=O)ncc23)C1] |
| | SM: 6-chloro-1-(2-(1,1-difluoroethyl)pyrimidin-4-yl)-3-(3- |
| | methoxypiperidin-1-yl)-1H-pyrazolo[4,3-c]pyridine (Preparation 298). |
| | HPLC-F (43-73% MeCN) |
| | Yellow solid (19 mg, 22%). LCMS m/z = 432.2 [M + H]⁺. ¹H NMR (400 |
| | MHz, CDC1₃) δ: 9.99 (s, 1H), 9.77 (s, 1H), 8.84 (d, 1H), 8.78 (s, 1H), 7.84 |
| | (d, 1H), 3.87-3.85 (m, 1H), 3.62-3.59 (m, 2H), 3.54-3.52 (m, 2H), 3.42 (s, |
| | 3H), 2.35-2.22 (m, 6H), 2.02-2.00 (s, 2H), 1.80-1.71 (m, 2H). |
| 175 | N-(1-(2-(1,1-difluoroethyl)pyrimidin-4-yl)-3-(4-methoxypiperidin-1-yl)- |
| | 1H-pyrazolo[4,3-c]pyridin-6-yl)acetamide |
| | 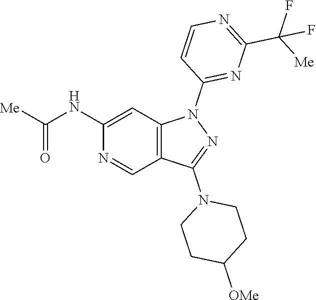 [SMILES: COC1CCN(c2nn(-c3ccnc(C(C)(F)F)n3)c3cc(NC(C)=O)ncc23)CC1] |
| | SM: 6-chloro-1-(2-(1,1-difluoroethyl)pyrimidin-4-yl)-3-(4- |
| | methoxypiperidin-1-yl)-1H-pyrazolo[4,3-c]pyridine (Preparation 356). |
| | HPLC-F (41-71% MeCN) |
| | White solid (5.7 mg, 11%). LCMS m/z = 431.2 [M + H]⁺. ¹H NMR (500 |
| | MHz, CDCl₃) δ: 9.52 (s, 1H), 8.77-8.75 (m, 2H), 8.10 (br s, 1H), 7.79 (d, |
| | 1H), 3.94-3.92 (m, 2H), 3.51-3.49 (m, 1H), 3.42-3.39 (m, 5H), 2.33-2.25 |
| | (m, 6H), 2.10-2.08 (m, 2H), 1.84-1.80 (m, 2H). |
| 176 | N-(1-(2-(1,1-difluoroethyl)pyrimidin-4-yl)-3-(pyrrolidin-1-yl)-1H- |
| | pyrazolo[4,3-c]pyridin-6-yl)acetamide |
| | 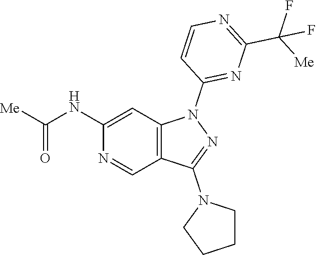 [SMILES: CC(=O)Nc1cc2c(cn1)c(N1CCCC1)nn2-c1ccnc(C(C)(F)F)n1] |
| | SM: 6-chloro-1-(2-(1,1-difluoroethyl)pyrimidin-4-yl)-3-(pyrrolidin-1-yl)- |
| | 1H-pyrazolo[4,3-c]pyridine (Preparation 357). |
| | HPLC-F (37-67% MeCN) |
| | White solid (33.3 mg, 39%). LCMS m/z = 338.1 [M + H]⁺. ¹H NMR (400 |
| | MHz, CDCl₃) δ: 9.68 (br s, 1H), 8.76 (d, 1H), 8.63 (s, 1H), 7.81 (d, 1H), |
| | 3.75-3.72 (m, 4H), 2.33-2.24 (m, 6H), 2.14-2.11 (m, 4H). |
| 177 | N-(1-(4-(1,1-difluoroethyl)pyrimidin-2-yl)-3-(3-methoxypyrrolidin-1-yl)- |
| | 1H-pyrazolo[4,3-c]pyridin-6-yl)acetamide |
| | 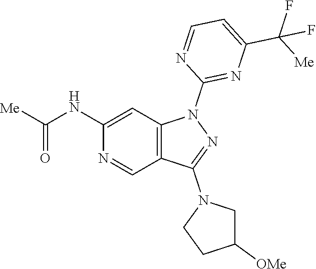 [SMILES: COC1CCN(c2nn(-c3nccc(C(C)(F)F)n3)c3cc(NC(C)=O)ncc23)C1] |
| | SM: 6-chloro-1-(4-(1,1-difluoroethyl)pyrimidin-2-yl)-3-(3- |
| | methoxypyrrolidin-1-yl)-1H-pyrazolo[4,3-c]pyridine (Preparation 299) |
| | HPLC-F (35-65% MeCN) |
| | LCMS m/z = 418.1 [M + H]⁺; ¹H NMR (500 MHz, CDCl₃) δ: 9.94 (br s, |
| | 1H), 9.57 (s, 1H), 9.01 (d, 1H), 8.73 (s, 1H), 7.46 (d, 1H), 4.19-4.18 (m, |
| | 1H), 3.93-3.87 (m, 4H), 3.40 (s, 3H), 2.33 (s, 3H), 2.27 (t, 3H), 2.22-2.19 |
| | (m, 2H). |
| 178 | N-(1-(2-(1,1-difluoroethyl)pyrimidin-4-yl)-3-(3-methoxypyrrolidin-1-yl)- |
| | 1H-pyrazolo[4,3-c]pyridin-6-yl)acetamide |
| | 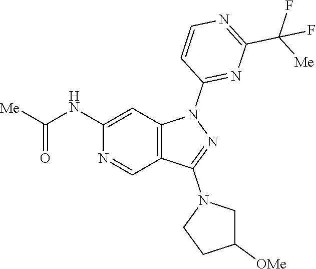 [SMILES: COC1CCN(c2nn(-c3ccnc(C(C)(F)F)n3)c3cc(NC(C)=O)ncc23)C1] |
| | SM: 6-chloro-1-(2-(1,1-difluoroethyl)pyrimidin-4-yl)-3-(3- |
| | methoxypyrrolidin-1-yl)-1H-pyrazolo[4,3-c]pyridine (Preparation 300). |
| | HPLC-F (41-71% MeCN) |
| | Yellow oil (18.5 mg, 18%). LCMS m/z = 418.1 [M + H]⁺. ¹H NMR (500 |
| | MHz, CDCl₃) δ: 9.68 (s, 1H), 9.57 (br s, 1H), 8.80 (d, 1H), 8.71 (s, 1H), |
| | 7.84 (d, 1H), 4.20-4.19 (m, 1H), 3.84-3.81 (m, 4H), 3.42 (s, 3H), 2.33 (s, |
| | 3H), 2.31-2.17 (m, 5H). |
| 179 | N-(1-(4-(1,1-difluoroethyl)pyrimidin-2-yl)-3-(3-hydroxypyrrolidin-1-yl)- |
| | 1H-pyrazolo[4,3-c]pyridin-6-yl)acetamide |
| | 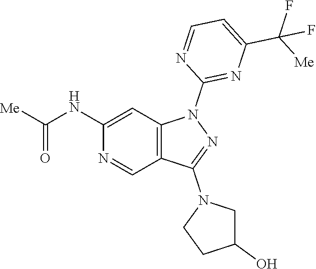 [SMILES: CC(=O)Nc1cc2c(cn1)c(N1CCC(O)C1)nn2-c1nccc(C(C)(F)F)n1] |
| | SM: 1-(6-chloro-1-(4-(1,1-difluoroethyl)pyrimidin-2-yl)-1H-pyrazolo[4,3- |
| | c]pyridin-3-yl)pyrrolidin-3-ol (Preparation 301) |
| | HPLC-C (23-53% MeCN). White solid (12.6 mg, 17%). LCMS m/z = |
| | 404.2 [M + H]⁺; ¹H NMR (500 MHz, CDCl₃) δ: 9.41 (s, 1H), 8.96 (d, 1H), |
| | 8.84 (s, 1H), 8.75 (s, 1H), 7.39 (d, 1H), 4.72-4.71 (m, 1H), 4.02-3.84 (m, |
| | 4H), 2.30-2.21 (m, 8H). |
| 180 | N-(1-(2-(1, 1-difluoroethyl)pyrimidin-4-yl)-3-(3-hydroxypyrrolidin-1-yl)- |
| | 1H-pyrazolo[4,3-c]pyridin-6-yl)acetamide |
| | 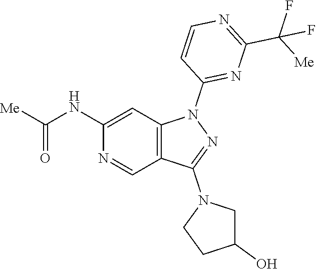 [SMILES: CC(=O)Nc1cc2c(cn1)c(N1CCC(O)C1)nn2-c1ccnc(C(C)(F)F)n1] |
| | SM: 1-(6-chloro-1-(2-(1,1-difluoroethyl)pyrimidin-4-yl)-1H-pyrazolo[4,3- |
| | c]pyridin-3-yl)pyrrolidin-3-ol (Preparation 302). |
| | HPLC-O (15-35% MeCN) |
| | White solid (6.9 mg, 8%). LCMS m/z = 404.1 [M + H]⁺. ¹H NMR (400 |
| | MHz, MeOH-d₄) δ: 9.32 (s, 1H), 8.84 (s, 1H), 8.71 (d, 1H), 7.80 (d, 1H), |
| | 3.88-3.83 (m, 4H), 3.70-3.67 (m, 1H), 2.28-2.14 (m, 8H). |
| 181 | N-(1-(4-(1,1-difluoroethyl)pyrimidin-2-yl)-3-(3-hydroxy-3- |
| | methylpyrrolidin-1-yl)-1H-pyrazolo[4,3-c]pyridin-6-yl)acetamide |
| | 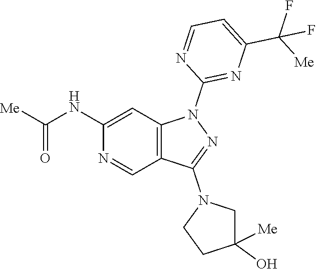 [SMILES: CC(=O)Nc1cc2c(cn1)c(N1CCC(C)(O)C1)nn2-c1nccc(C(C)(F)F)n1] |
| | SM: 1-(6-chloro-1-(4-(1,1-difluoroethyl)pyrimidin-2-yl)-1H-pyrazolo[4,3- |
| | c]pyridin-3-yl)-3-methylpyrrolidin-3-ol (Preparation 303) |
| | White solid (25.5 mg, 34%). LCMS m/z = 418.1 [M + H]⁺; ¹H NMR (500 |
| | MHz, MeOH-d₄) δ: 9.33 (s, 1H), 8.96 (d, 1H), 8.89 (s, 1H), 7.47 (d, 1H), |
| | 4.01-3.98 (m, 2H), 3.94-3.92 (m, 1H), 3.80-3.69 (m, 1H), 2.28-2.23 (m, |
| | 3H), 2.19 (s, 3H), 2.13-2.09 (m, 2H), 1.53 (s, 3H). |
| 182 | N-(1-(2-(1,1-difluoroethyl)pyrimidin-4-yl)-3-(3-hydroxy-3- |
| | methylpyrrolidin-1-yl)-1H-pyrazolo[4,3-c]pyridin-6-yl)acetamide |
| | 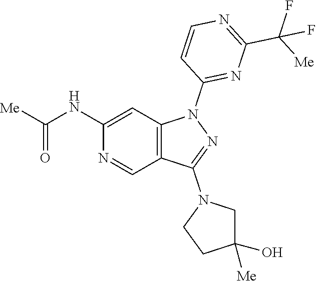 [SMILES: CC(=O)Nc1cc2c(cn1)c(N1CCC(C)(O)C1)nn2-c1ccnc(C(C)(F)F)n1] |
| | SM: 1-(6-chloro-1-(2-(1,1-difluoroethyl)pyrimidin-4-yl)-1H-pyrazolo[4,3- |
| | c]pyridin-3-yl)-3-methylpyrrolidin-3-ol (Preparation 304). HPLC-F (40- |
| | 70% MeCN). White solid (33.5 mg, 32%). LCMS m/z = 418.1 [M + H]⁺. ¹H |
| | NMR (400 MHz, MeOH-d₄) δ: 9.40 (s, 1H), 8.87 (s, 1H), 8.73 (d, 1H), 7.85 |
| | (d, 1H), 3.96-3.89 (m, 2H), 3.77-3.65 (m, 2H), 2.29 (s, 3H), 2.23-2.19 (m, |
| | 3H), 2.13-2.09 (m, 2H), 1.52 (s, 3H). |
| 183 | N-(3-(3-cyano-3-fluoropyrrolidin-1-yl)-1-(2-(1,1-difluoroethyl)pyrimidin- |
| | 4-yl)-1H-pyrazolo[4,3-c]pyridin-6-yl)acetamide |
| | 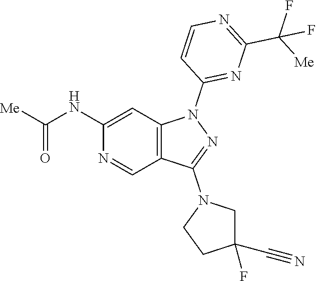 [SMILES: CC(=O)Nc1cc2c(cn1)c(N1CCC(F)(C#N)C1)nn2-c1ccnc(C(C)(F)F)n1] |
| | SM: 1-(6-chloro-1-(2-(1,1-difluoroethyl)pyrimidin-4-yl)-1H-pyrazolo[4,3- |
| | c]pyridin-3-yl)-3-fluoropyrrolidine-3-carbonitrile (Preparation 305) |
| | HPLC-F (41-71% MeCN) |
| | White solid (3.1 mg, 29%). LCMS m/z = 431.1 [M + H]⁺; ¹H NMR (400 |
| | MHz, CDCl₃) δ: 9.53 (s, 1H), 8.77 (d, 1H), 8.71 (s, 1H), 8.12 (s, 1H), 7.78 |
| | (d, 1H), 4.38-4.21 (m, 2H), 4.14-4.13 (m, 1H), 4.04-4.01 (m, 1H), 2.88-2.82 |
| | (m, 1H), 2.76-2.64 (m, 1H), 2.33-2.23 (m, 6H). |
| 184 | N-(3-(3-cyano-3-methylpyrrolidin-1-yl)-1-(4-(1,1-difluoroethyl)pyrimidin- |
| | 2-yl)-1H-pyrazolo[4,3-c]pyridin-6-yl)acetamide |
| | 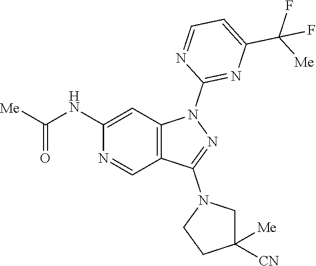 [SMILES: CC(=O)Nc1cc2c(cn1)c(N1CCC(C)(C#N)C1)nn2-c1nccc(C(C)(F)F)n1] |
| | SM: 1-(6-chloro-1-(4-(1,1-difluoroethyl)pyrimidin-2-yl)-1H-pyrazolo[4,3- |
| | c]pyridin-3-yl)-3-methylpyrrolidine-3-carbonitrile (Preparation 306) |
| | HPLC-C (30-60% MeCN). White solid (42 mg, 44%). LCMS m/z = 427.1 |
| | [M + H]⁺; ¹H NMR (400 MHz, CDCl₃) δ: 9.98 (br s, 1H), 9.61 (s, 1H), 9.02 |
| | (d, 1H), 8.75 (s, 1H), 7.49 (d, 1H), 4.30 (d, J), 4.14-4.01 (m, 2H), 3.73 (d, |
| | 1H), 2.67-2.64 (m, 1H), 2.34-2.18 (m, 7H), 1.66 (s, 3H). |
| 185 | N-(3-(3-cyano-3-methylpyrrolidin-1-yl)-1-(2-(1,1-difluoroethyl)pyrimidin- |
| | 4-yl)-1H-pyrazolo[4,3-c]pyridin-6-yl)acetamide |
| | 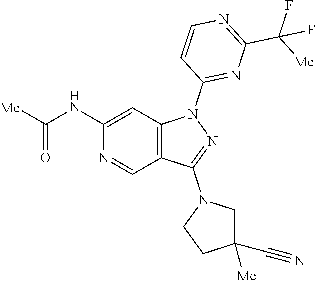 [SMILES: CC(=O)Nc1cc2c(cn1)c(N1CCC(C)(C#N)C1)nn2-c1ccnc(C(C)(F)F)n1] |
| | SM: 1-(6-chloro-1-(2-(1,1-difluoroethyl)pyrimidin-4-yl)-1H-pyrazolo[4,3- |
| | c]pyridin-3-y1)-3-methylpyrrolidine-3-carbonitrile (Preparation 307) |
| | HPLC-C (33-63% MeCN). |
| | White solid (16 mg, 22%). LCMS m/z = 427.1 [M + H]⁺; ¹H NMR (400 |
| | MHz, CDCl₃) δ: 9.66 (s, 1H), 9.23 (br s, 1H), 8.81 (d, 1H), 8.74 (s, 1H), |
| | 7.80 (d, 1H), 4.22 (d, 1H), 4.07-3.97 (m, 2H), 3.64 (d, 1H), 2.67-2.64 (m, |
| | 1H), 2.32-2.18 (m, 7H), 1.67 (s, 3H). |
| 186 | N-(3-(azetidin-1-yl)-1-(4-(1,1-difluoroethyl)pyrimidin-2-yl)-1H- |
| | pyrazolo[4,3-c]pyridin-6-yl)acetamide |
| | 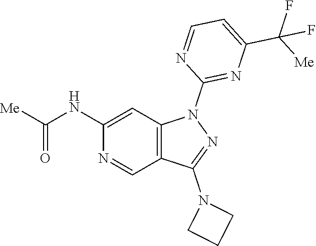 [SMILES: CC(=O)Nc1cc2c(cn1)c(N1CCC1)nn2-c1nccc(C(C)(F)F)n1] |
| | SM: 3-(azetidin-1-yl)-6-chloro-1-(4-(1,1-difluoroethyl)pyrimidin-2-yl)-1H- |
| | pyrazolo[4,3-c]pyridine (Preparation 308) |
| | HPLC-C (27-57% MeCN). |
| | White solid (25.4 mg, 34%). LCMS m/z = 374.1 [M + H]⁺; ¹H NMR (500 |
| | MHz, CDCl₃) δ: 9.34 (s, 1H), 8.94 (d, 1H), 8.55 (s, 1H), 8.15 (s, 1H), 7.36 |
| | (d, 1H), 4.42-4.39 (m, 4H), 2.60-2.54 (m, 2H), 2.32-2.24 (m, 6H). |
| 187 | N-(3-(3,3-difluoroazetidin-1-yl)-1-(4-(1,1-difluoroethyl)pyrimidin-2-yl)- |
| | 1H-pyrazolo[4,3-c]pyridin-6-yl)acetamide |
| | 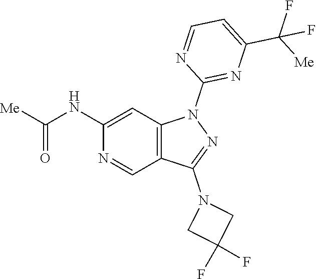 [SMILES: CC(=O)Nc1cc2c(cn1)c(N1CC(F)(F)C1)nn2-c1nccc(C(C)(F)F)n1] |
| | SM: 6-chloro-3-(3,3-difluoroazetidin-1-yl)-1-(4-(1,1- |
| | difluoroethyl)pyrimidin-2-yl)-1H-pyrazolo[4,3-c]pyridine (Preparation 309) |
| | HPLC-C (33-63% MeCN). Pink solid (10 mg, 26%). LCMS m/z = 410.1 |
| | [M + H]⁺; ¹H NMR (500 MHz, MeOH-d₄) δ: 9.34 (s, 1H), 8.99 (d, 1H), 8.71 |
| | (s, 1H), 7.54 (d, 1H), 4.78-4.70 (m, 4H), 2.30-2.16 (m, 6H). |
| 188 | N-(3-(3,3-difluoroazetidin-1-yl)-1-(2-(1,1-difluoroethyl)pyrimidin-4-yl)- |
| | 1H-pyrazolo[4,3-c]pyridin-6-yl)acetamide |
| | 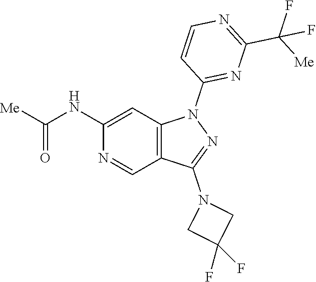 [SMILES: CC(=O)Nc1cc2c(cn1)c(N1CC(F)(F)C1)nn2-c1ccnc(C(C)(F)F)n1] |
| | SM: 6-chloro-3-(3,3-difluoroazetidin-1-yl)-1-(2-(1,1- |
| | difluoroethyl)pyrimidin-4-yl)-1H-pyrazolo[4,3-c]pyridine (Preparation 310) |
| | HPLC-C (38-48% MeCN). Yellow solid (7.1 mg, 7.5%). LCMS m/z = |
| | 410.1 [M + H]⁺; ¹H NMR (500 MHz, DMSO-d₆) δ: 10.80 (s, 1H), 9.36 (s, |
| | 1H), 8.89 (d, 1H), 8.81 (s, 1H), 7.80 (d, 1H), 4.88-4.70 (m, 4H), 2.22 (t, |
| | 3H), 2.16 (s, 3H). |
| 189 | N-(1-(4-(1,1-difluoroethyl)pyrimidin-2-yl)-3-(3-fluoroazetidin-1-yl)-1H- |
| | pyrazolo[4,3-c]pyridin-6-yl)acetamide |
| | 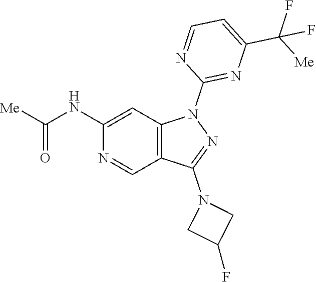 [SMILES: CC(=O)Nc1cc2c(cn1)c(N1CC(F)C1)nn2-c1nccc(C(C)(F)F)n1] |
| | SM: 6-chloro-1-(4-(1,1-difluoroethyl)pyrimidin-2-yl)-3-(3-fluoroazetidin-1- |
| | yl)-1H-pyrazolo[4,3-c]pyridine (Preparation 311). HPLC-C (28-58% |
| | MeCN). |
| | Yellow solid (24 mg, 20%). LCMS m/z = 392.1 [M + H]⁺; ¹H NMR (500 |
| | MHz, CDCl₃) δ: 9.39 (s, 1H), 8.97 (br s, 1H), 8.55 (s, 1H), 8.33 (s, 1H), |
| | 7.51-7.35 (m, 1H), 5.70-5.40 (m, 1H), 4.79-4.43 (m, 4H), 2.32-2.23 (m, |
| | 6H). |
| 190 | N-(1-(2-(1,1-difluoroethyl)pyrimidin-4-yl)-3-(3-fluoroazetidin-1-yl)-1H- |
| | pyrazolo[4,3-c]pyridin-6-yl)acetamide |
| | 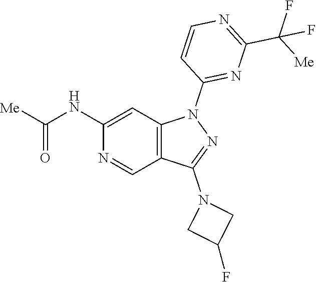 [SMILES: CC(=O)Nc1cc2c(cn1)c(N1CC(F)C1)nn2-c1ccnc(C(C)(F)F)n1] |
| | SM: 6-chloro-1-(2-(1,1-difluoroethyl)pyrimidin-4-yl)-3-(3-fluoroazetidin-1- |
| | yl)-1H-pyrazolo[4,3-c]pyridine (Preparation 312). HPLC-C (30-60% |
| | MeCN). |
| | White solid (26 mg, 41%). LCMS m/z = 392.1 [M + H]⁺; ¹H NMR (500 |
| | MHz, DMSO-d₆) δ: 10.77 (s, 1H), 9.35 (s, 1H), 8.86 (d, 1H), 8.79 (d, 1H), |
| | 7.78 (d, 1H), 5.71-5.52 (m, 1H), 4.69-4.56 (m, 2H), 4.46-4.34 (m, 2H), 2.22 |
| | (t, 3H), 2.16 (s, 3H). |
| 191 | N-(1-(4-(1,1-difluoroethyl)pyrimidin-2-yl)-3-(2-methylazetidin-1-yl)-1H- |
| | pyrazolo[4,3-c]pyridin-6-yl)acetamide |
| | 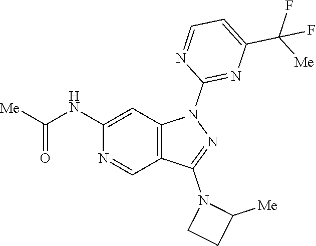 [SMILES: CC(=O)Nc1cc2c(cn1)c(N1CCC1C)nn2-c1nccc(C(C)(F)F)n1] |
| | SM: 6-chloro-1-(4-(1,1-difluoroethyl)pyrimidin-2-yl)-3-(2-methylazetidin- |
| | 1-yl)-1H-pyrazolo[4,3-c]pyridine (Preparation 313). HPLC-F (40-70% |
| | MeCN) |
| | White solid (10 mg, 27%). LCMS m/z = 388.1 [M + H]⁺; ¹H NMR (500 |
| | MHz, MeOH-d₄) δ: 9.34 (s, 1H), 8.98 (d, 1H), 8.70 (s, 1H), 7.52 (d, 1H), |
| | 4.78-4.75 (m, 1H), 4.42-4.39 (m, 1H), 4.23-4.19 (m, 1H), 2.69-2.66 (m, |
| | 1H), 2.28-2.21 (m, 7H), 1.63 (d, 3H). |
| 192 | N-(1-(4-(1,1-difluoroethyl)pyrimidin-2-yl)-3-(3-methoxyazetidin-1-yl)-1H- |
| | pyrazolo[4,3-c]pyridin-6-yl)acetamide |
| | 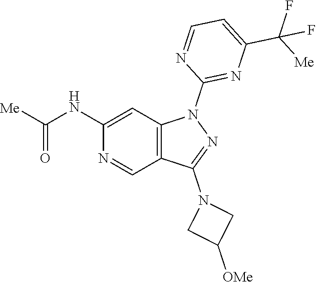 [SMILES: COC1CN(c2nn(-c3nccc(C(C)(F)F)n3)c3cc(NC(C)=O)ncc23)C1] |
| | SM: 6-chloro-1-(4-(1,1-difluoroethyl)pyrimidin-2-yl)-3-(3- |
| | methoxyazetidin-1-yl)-1H-pyrazolo[4,3-c]pyridine (Preparation 314). |
| | HPLC-C (29-52% MeCN). |
| | White solid (17 mg, 32%). LCMS m/z = 404.0 [M + H]⁺; ¹H NMR (500 |
| | MHz, CDCl₃) δ: 9.37 (s, 1H), 8.96 (d, 1H), 8.54 (s, 1H), 8.37 (s, 1H), 7.39 |
| | (d, 1H), 4.58-4.55 (m, 2H), 4.48-4.47 (m, 1H), 4.28-4.25 (m, 2H), 3.37 (s, |
| | 3H), 2.28 (t, 3H), 2.27 (s, 3H). |
| 193 | N-(1-(2-(1,1-difluoroethyl)pyrimidin-4-yl)-3-(3-methoxyazetidin-1-yl)-1H- |
| | pyrazolo[4,3-c]pyridin-6-yl)acetamide |
| | 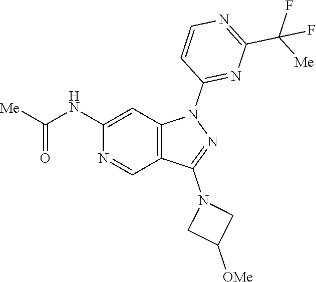 [SMILES: COC1CN(c2nn(-c3ccnc(C(C)(F)F)n3)c3cc(NC(C)=O)ncc23)C1] |
| | SM: 6-chloro-1-(2-(1,1-difluoroethyl)pyrimidin-4-yl)-3-(3- |
| | methoxyazetidin-1-yl)-1H-pyrazolo[4,3-c]pyridine (Preparation 315). |
| | HPLC-C (31-61% MeCN). |
| | White solid (17 mg, 32%). LCMS m/z = 404.1 [M + H]⁺; ¹H NMR (400 |
| | MHz, CDCl₃) δ: 9.47 (s, 1H), 8.75 (d, 1H), 8.53 (s, 1H), 8.23 (s, 1H), 7.76 |
| | (d, 1H), 4.49 (s, 3H), 4.21-4.19 (m, 2H), 3.39 (s, 3H), 2.33-2.23 (m, 6H). |
| 194 | N-(1-(4-(1,1-difluoroethyl)pyrimidin-2-yl)-3-(3-hydroxyazetidin-1-yl)-1H- |
| | pyrazolo[4,3-c]pyridin-6-yl)acetamide |
| | 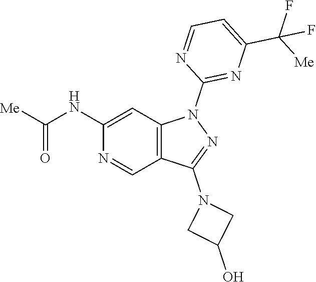 [SMILES: CC(=O)Nc1cc2c(cn1)c(N1CC(O)C1)nn2-c1nccc(C(C)(F)F)n1] |
| | SM: 1-(6-chloro-1-(4-(1,1-difluoroethyl)pyrimidin-2-yl)-1H-pyrazolo[4,3- |
| | c]pyridin-3-yl)azetidin-3-ol (Preparation 316). HPLC-C (20-50% MeCN). |
| | White solid (5 mg, 16%). LCMS m/z = 390.1 [M + H]⁺; ¹H NMR (500 MHz, |
| | DMSO-d₆) δ: 10.73 (s, 1H), 9.25 (s, 1H), 9.04 (d, 1H), 8.76 (s, 1H), 7.55 (d, |
| | 1H), 5.81 (d, 1H), 4.74-4.69 (m, 1H), 4.52-4.48 (m, 2H), 4.03-4.00 (m, 2H), |
| | 2.23 (t, 3H), 2.16 (s, 3H). |
| 195 | N-(1-(2-(1,1-difluoroethyl)pyrimidin-4-yl)-3-(3-hydroxyazetidin-1-yl)-1H- |
| | pyrazolo[4,3-c]pyridin-6-yl)acetamide |
| | 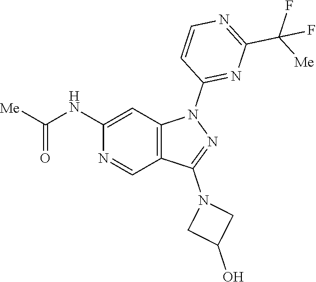 [SMILES: CC(=O)Nc1cc2c(cn1)c(N1CC(O)C1)nn2-c1ccnc(C(C)(F)F)n1] |
| | SM: 1-(6-chloro-1-(2-(1,1-difluoroethyl)pyrimidin-4-yl)-1H-pyrazolo[4,3- |
| | c]pyridin-3-yl)azetidin-3-ol (Preparation 317). HPLC-C (33-63% MeCN). |
| | Yellow solid (15 mg, 12%). LCMS m/z = 390.1 [M + H]⁺; ¹H NMR (400 |
| | MHz, DMSO-d₆) δ: 10.73 (s, 1H), 9.30 (s, 1H), 8.80 (d, 1H), 8.72 (s, 1H), |
| | 7.71 (d, 1H), 4.70-4.65 (m, 1H), 4.49-4.45 (m, 2H), 4.02-3.98 (m, 2H), 2.19 |
| | (t, 3H), 2.12 (s, 3H). |
| 196 | N-(1-(4-(1,1-difluoroethyl)pyrimidin-2-yl)-3-(3-hydroxy-3-methylazetidin- |
| | 1-yl)-1H-pyrazolo[4,3-c]pyridin-6-yl)acetamide |
| | 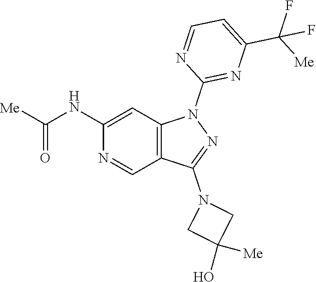 [SMILES: CC(=O)Nc1cc2c(cn1)c(N1CC(C)(O)C1)nn2-c1nccc(C(C)(F)F)n1] |
| | SM: 1-(6-chloro-1-(4-(1,1-difluoroethyl)pyrimidin-2-yl)-1H-pyrazolo[4,3- |
| | c]pyridin-3-yl)-3-methylazetidin-3-ol (Preparation 318). HPLC-C (25-55% |
| | MeCN). White solid (25 mg, 39%). LCMS m/z = 404.1 [M + H]⁺; ¹H NMR |
| | (500 MHz, CDCl₃) δ: 9.49 (s, 1H), 8.98 (d, 1H), 8.52 (s, 1H), 7.46 (d, 1H), |
| | 4.33 (s, 4H), 2.32-2.22 (m, 6H), 1.70 (s, 3H). |
| 197 | N-(1-(2-(1,1-difluoroethyl)pyrimidin-4-yl)-3-(3-hydroxy-3-methylazetidin- |
| | 1-yl)-1H-pyrazolo[4,3-c]pyridin-6-yl)acetamide |
| | 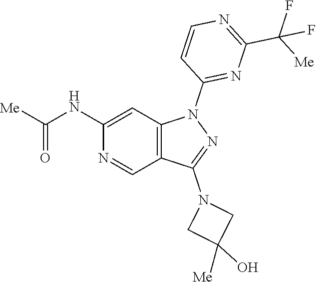 [SMILES: CC(=O)Nc1cc2c(cn1)c(N1CC(C)(O)C1)nn2-c1ccnc(C(C)(F)F)n1] |
| | SM: 1-(6-chloro-1-(2-(1,1-difluoroethyl)pyrimidin-4-yl)-1H-pyrazolo[4,3- |
| | c]pyridin-3-yl)-3-methylazetidin-3-ol (Preparation 319). HPLC-C (29-59% |
| | MeCN). White solid (20 mg, 38%). LCMS m/z = 404.1 [M + H]⁺; ¹H NMR |
| | (500 MHz, CDCl₃) δ: 9.54 (s, 1H), 8.97 (d, 1H), 8.50 (s, 1H), 7.77 (d, 1H), |
| | 4.25 (s, 4H), 2.31-2.23 (m, 6H), 1.71 (s, 3H). |
| 198 | N-(1-(4-(1,1-difluoroethyl)pyrimidin-2-yl)-3-(3-methoxy-3-methylazetidin- |
| | 1-yl)-1H-pyrazolo[4,3-c]pyridin-6-yl)acetamide |
| | 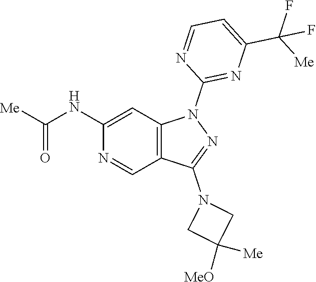 [SMILES: COC1(C)CN(c2nn(-c3nccc(C(C)(F)F)n3)c3cc(NC(C)=O)ncc23)C1] |
| | SM: 6-chloro-1-(4-(1,1-difluoroethyl)pyrimidin-2-yl)-3-(3-methoxy-3- |
| | methylazetidin-1-yl)-1H-pyrazolo[4,3-c]pyridine (Preparation 320) |
| | HPLC-F (43-73% MeCN) |
| | Yellow solid (23 mg, 36%). LCMS m/z = 418.1 [M + H]⁺; ¹H NMR (500 |
| | MHz, CDCl₃) δ: 9.36 (s, 1H), 8.95 (d, 1H), 8.55 (s, 1H), 8.24 (s, 1H), 7.38 |
| | (d, 1H), 4.36 (d, 2H), 4.16 (d, 2H), 3.32 (s, 3H), 2.31-2.14 (m, 6H), 1.62 (s, |
| | 3H). |
| 199 | N-(1-(2-(1,1-difluoroethyl)pyrimidin-4-yl)-3-(3-methoxy-3-methylazetidin- |
| | 1-yl)-1H-pyrazolo[4,3-c]pyridin-6-yl)acetamide |
| | 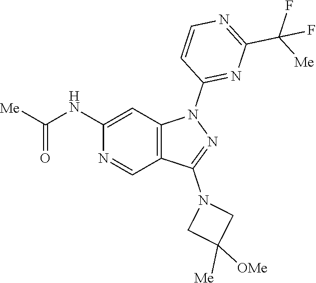 [SMILES: COC1(C)CN(c2nn(-c3ccnc(C(C)(F)F)n3)c3cc(NC(C)=O)ncc23)C1] |
| | SM: 6-chloro-1-(2-(1,1-difluoroethyl)pyrimidin-4-yl)-3-(3-methoxy-3- |
| | methylazetidin-1-yl)-1H-pyrazolo[4,3-c]pyridine (Preparation 321) |
| | HPLC-F (41-71% MeCN) |
| | White solid (13 mg, 25%). LCMS m/z = 418.2 [M + H]⁺; ¹H NMR (400 |
| | MHz, MeOH-d₄) δ: 9.36 (s, 1H), 8.73 (d, 1H), 8.66 (s, 1H), 7.82 (d, 1H), |
| | 4.25 (d, 2H), 4.12 (d, 2H), 3.31 (s, 3H), 2.25-2.16 (m, 6H), 1.59 (s, 3H). |
| 200 | N-(1-(4-(1,1-difluoroethyl)pyrimidin-2-yl)-3-(3-(trifluoromethoxy)azetidin- |
| | 1-yl)-1H-pyrazolo[4,3-c]pyridin-6-yl)acetamide |
| | 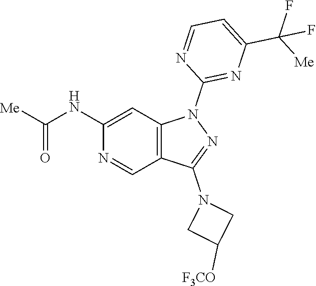 [SMILES: CC(=O)Nc1cc2c(cn1)c(N1CC(OC(F)(F)F)C1)nn2-c1nccc(C(C)(F)F)n1] |
| | SM: 6-chloro-1-(4-(1,1-difluoroethyl)pyrimidin-2-yl)-3-(3- |
| | (trifluoromethoxy)azetidin-1-yl)-1H-pyrazolo[4,3-c]pyridine (Preparation |
| | 322) |
| | HPLC-F (45-75% MeCN) |
| | White solid (6 mg, 6%). LCMS m/z = 458.0 [M + H]⁺; ¹H NMR (400 MHz, |
| | MeOH-d₄) δ: 9.32 (s, 1H), 8.98 (d, 1H), 8.70 (s, 1H), 7.52 (d, 1H), 5.40- |
| | 5.34 (m, 1H), 4.76-4.71 (m, 2H), 4.46-4.42 (m, 2H), 2.28-2.17 (m, 6H). |
| 201 | N-(1-(2-(1,1-difluoroethyl)pyrimidin-4-yl)-3-(3-(trifluoromethoxy)azetidin- |
| | 1-yl)-1H-pyrazolo[4,3-c]pyridin-6-yl)acetamide |
| | 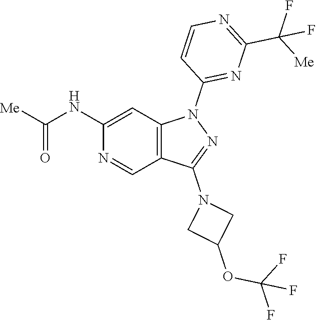 [SMILES: CC(=O)Nc1cc2c(cn1)c(N1CC(OC(F)(F)F)C1)nn2-c1ccnc(C(C)(F)F)n1] |
| | SM: 6-chloro-1-(2-(1,1-difluoroethyl)pyrimidin-4-yl)-3-(3- |
| | (trifluoromethoxy)azetidin-1-yl)-1H-pyrazolo[4,3-c]pyridine (Preparation |
| | 323) |
| | HPLC-F (47-67% MeCN) |
| | White solid (26 mg, 41%). LCMS m/z = 458.1 [M + H]⁺; ¹H NMR (400 |
| | MHz, MeOH-d₄) δ: 9.40 (s, 1H), 8.77 (d, 1H), 8.69 (d, 1H), 7.86 (d, 1H), |
| | 5.38 (s, 1H), 4.74-4.69 (m, 2H), 4.44-4.40 (m, 2H) 2.29-2.19 (m, 6H). |
| 202 | N-(1-(4-(1,1-difluoroethyl)pyrimidin-2-yl)-3-(3-(dimethylamino)azetidin-1- |
| | yl)-1H-pyrazolo[4,3-c]pyridin-6-yl)acetamide |
| | 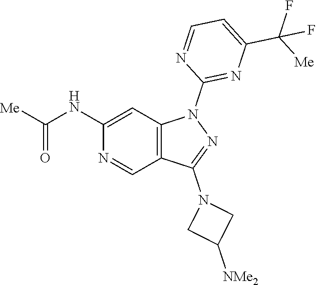 [SMILES: CC(=O)Nc1cc2c(cn1)c(N1CC(N(C)C)C1)nn2-c1nccc(C(C)(F)F)n1] |
| | SM: 1-(6-chloro-1-(4-(1,1-difluoroethyl)pyrimidin-2-yl)-1H-pyrazolo[4,3- |
| | c]pyridin-3-yl)-N,N-dimethylazetidin-3-amine (Preparation 324) |
| | HPLC-C (26-56% MeCN). White solid (34.1 mg, 40%). LCMS m/z = |
| | 417.1 [M + H]⁺; ¹H NMR (500 MHz, CDCl₃) δ: 9.34 (s, 1H), 8.95 (d, 1H), |
| | 8.56 (s, 1H), 8.11 (s, 1H), 7.38 (d, 1H), 4.75-4.44 (m, 2H), 4.38-4.28 (m, |
| | 2H), 3.58-3.49 (m, 1H), 2.36 (s, 6H), 2.28 (t, 1H), 2.26 (s, 3H). |
| 203 | N-(1-(2-(1,1-difluoroethyl)pyrimidin-4-yl)-3-(3-(dimethylamino)azetidin-1- |
| | yl)-1H-pyrazolo[4,3-c]pyridin-6-yl)acetamide |
| | 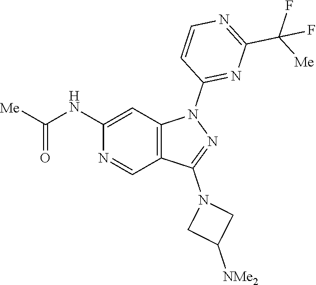 [SMILES: CC(=O)Nc1cc2c(cn1)c(N1CC(N(C)C)C1)nn2-c1ccnc(C(C)(F)F)n1] |
| | SM: 1-(6-chloro-1-(2-(1,1-difluoroethyl)pyrimidin-4-yl)-1H-pyrazolo[4,3- |
| | c]pyridin-3-yl)-N,N-dimethylazetidin-3-amine (Preparation 325) |
| | HPLC-C (25-55% MeCN). White solid (33.1 mg, 52%). LCMS m/z = |
| | 417.1 [M + H]⁺; ¹H NMR (500 MHz, MeOH-d₄) δ: 9.35 (s, 1H), 8.75 (d, |
| | 1H), 8.68 (s, 1H), 7.82 (d, 1H), 4.42-4.38 (m, 2H), 4.17-4.14 (m, 2H), 3.48- |
| | 3.43 (m, 1H), 2.28 (s, 6H), 2.24 (t, 3H), 2.23 (s, 3H). |
| 204 | N-(1-(4-(1,1-difluoroethyl)pyrimidin-2-yl)-3-(2-oxa-6-azaspiro[3.3]heptan- |
| | 6-yl)-1H-pyrazolo[4,3-c]pyridin-6-yl)acetamide |
| | 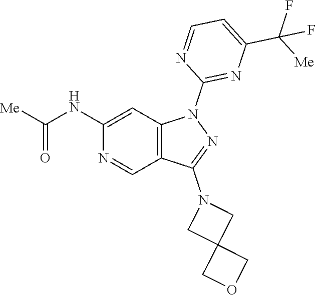 [SMILES: CC(=O)Nc1cc2c(cn1)c(N1CC3(COC3)C1)nn2-c1nccc(C(C)(F)F)n1] |
| | SM: 6-(6-chloro-1-(4-(1, 1-difluoroethyl)pyrimidin-2-yl)-1H-pyrazolo[4,3- |
| | c]pyridin-3-yl)-2-oxa-6-azaspiro[3.3]heptane (Preparation 326) |
| | HPLC-F (26-50% MeCN) Yellow solid (57.9 mg, 50%). LCMS m/z = |
| | 416.1 [M + H]⁺; ¹H NMR (400 MHz, CDCl₃) δ: 9.44 (s, 1H), 8.99 (d, 1H), |
| | 8.93 (br s, 1H), 8.55 (s, 1H), 7.44 (d, 1H), 4.91 (s, 4H), 4.55 (s, 4H), 2.31- |
| | 2.22 (m, 6H). |
| 205 | N-(1-(2-(1,1-difluoroethyl)pyrimidin-4-yl)-3-(2-oxa-6-azaspiro[3.3]heptan- |
| | 6-yl)-1H-pyrazolo[4,3-c]pyridin-6-yl)acetamide |
| | 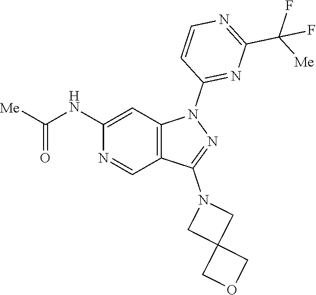 [SMILES: CC(=O)Nc1cc2c(cn1)c(N1CC3(COC3)C1)nn2-c1ccnc(C(C)(F)F)n1] |
| | SM: 6-(6-chloro-1-(2-(1,1-difluoroethyl)pyrimidin-4-yl)-1H-pyrazolo[4,3- |
| | c]pyridin-3-yl)-2-oxa-6-azaspiro[3.3]heptane (Preparation 327) |
| | HPLC-F (30-60% MeCN) Yellow solid (16.2 mg, 17%). LCMS m/z = |
| | 416.1 [M+H]⁺; ¹H NMR (500 MHz, CDCl₃) δ: 11.12 (s, 1H), 9.88 (s, 1H), |
| | 8.95-8.86 (m, 1H), 8.53 (s, 1H), 7.86 (d, 1H), 4.93 (s, 4H), 4.51 (s, 4H), |
| | 2.39 (s, 3H), 2.24 (t, 3H). |
| 206 | N-(3-(3-cyano-3-fluoroazetidin-1-yl)-1-(4-(1,1-difluoroethyl)pyrimidin-2- |
| | yl)-1H-pyrazolo[4,3-c]pyridin-6-yl)acetamide |
| | 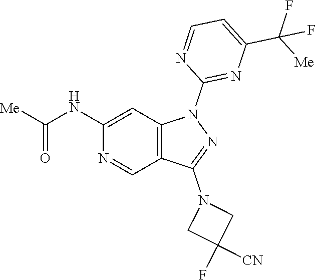 [SMILES: CC(=O)Nc1cc2c(cn1)c(N1CC(F)(C#N)C1)nn2-c1nccc(C(C)(F)F)n1] |
| | SM: 1-(6-chloro-1-(4-(1,1-difluoroethyl)pyrimidin-2-yl)-1H-pyrazolo[4,3- |
| | c]pyridin-3-yl)-3-fluoroazetidine-3-carbonitrile (Preparation 362) |
| | HPLC-C (27-57% MeCN). White solid (3 mg, 11%). LCMS m/z = 417.0 |
| | [M + H]⁺; ¹H NMR (400 MHz, CDCl₃) δ: 9.39 (s, 1H), 8.98 (d, 1H), 8.52 (s, |
| | 1H), 8.12 (s, 1H), 7.45 (d, 1H), 4.95-4.87 (m, 2H), 4.80-4.71 (m, 2H), 2.33- |
| | 2.23 (m, 6H). |
| 207 | N-(3-(azetidin-1-yl)-1-(6-(1,1-difluoroethyl)pyrimidin-4-yl)-1H- |
| | pyrazolo[4,3-c]pyridin-6-yl)acetamide |
| | 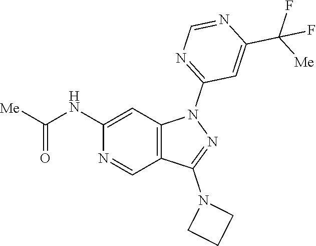 [SMILES: CC(=O)Nc1cc2c(cn1)c(N1CCC1)nn2-c1cc(C(C)(F)F)ncn1] |
| | SM: 3-(azetidin-1-yl)-6-chloro-1-(6-(1,1-difluoroethyl)pyrimidin-4-yl)-1H- |
| | pyrazolo[4,3-c]pyridine (Preparation 328) |
| | HPLC-C (36-66% MeCN). White solid (5.1 mg, 6%). LCMS m/z = 374.0 |
| | [M + H]⁺; ¹H NMR (400 MHz, CDCl₃) δ: 9.43 (s, 1H), 9.09 (s, 1H), 8.52 (s, |
| | 1H), 8.15 (s, 1H), 8.00 (s, 1H), 4.36-4.33 (m, 4H), 2.63-2.55 (m, 2H), 2.27 |
| | (s, 3H), 2.02 (t, 3H). |
| 208 | N-(1-(6-(1, 1-difluoroethyl)pyrimidin-4-yl)-3-morpholino-1H-pyrazolo[4,3- |
| | c]pyridin-6-yl)acetamide |
| | 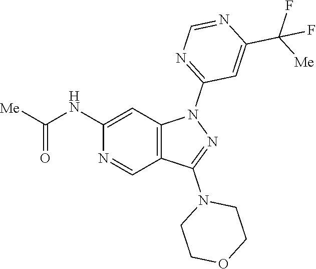 [SMILES: CC(=O)Nc1cc2c(cn1)c(N1CCOCC1)nn2-c1cc(C(C)(F)F)ncn1] |
| | SM: 4-(6-chloro-1-(6-(1,1-difluoroethyl)pyrimidin-4-yl)-1H-pyrazolo[4,3- |
| | c]pyridin-3-yl)morpholine (Preparation 329) |
| | HPLC-C (36-66% MeCN). White solid (50 mg, 47%). LCMS m/z = 404.1 |
| | [M + H]⁺; ¹H NMR (500 MHz, CDCl₃) δ: 9.57 (s, 1H), 9.13 (s, 1H), 8.76 (s, |
| | 1H), 8.45 (s, 1H), 8.02 (s, 1H), 3.96-3.94 (m, 4H), 3.65-3.63 (m, 4H), 2.29 |
| | (s, 3H), 2.03 (t, 3H). |
| 209 | N-(3-(azetidin-1-yl)-1-(6-(1,1-difluoroethyl)pyrimidin-4-yl)-1H- |
| | pyrrolo[3,2-c]pyridin-6-yl)acetamide |
| | 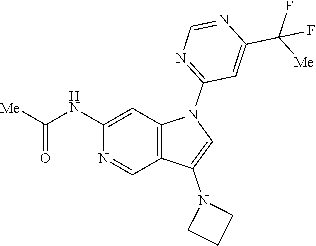 [SMILES: CC(=O)Nc1cc2c(cn1)c(N1CCC1)cn2-c1cc(C(C)(F)F)ncn1] |
| | SM: 3-(azetidin-1-yl)-6-chloro-1-(6-(1,1-difluoroethyl)pyrimidin-4-yl)-1H- |
| | pyrrolo[3,2-c]pyridine (Preparation 361) |
| | HPLC-F (35-65% MeCN) Yellow solid (6.8 mg, 18%). LCMS m/z = 373.1 |
| | [M + H]⁺; ¹H NMR (500 MHz, CDCl₃) δ: 9.41 (s, 1H), 9.14 (s, 1H), 8.47 (s, |
| | 1H), 8.10 (s, 1H), 7.54 (s, 1H), 6.80 (s, 1H), 4.08-4.05 (m, 4H), 2.52-2.46 |
| | (m, 2H), 2.26 (s 3H), 2.08-2.01 (m, 3H). |
| 210 | N-(1-(6-(1,1-difluoroethyl)pyrimidin-4-yl)-3-morpholino-1H-pyrrolo[3,2- |
| | c]pyridin-6-yl)acetamide |
| | 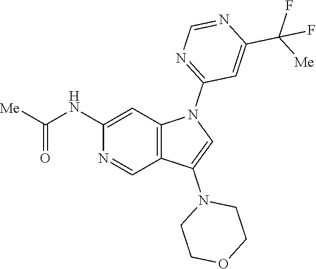 [SMILES: CC(=O)Nc1cc2c(cn1)c(N1CCOCC1)cn2-c1cc(C(C)(F)F)ncn1] |
| | SM: 4-(6-chloro-1-(6-(1,1-difluoroethyl)pyrimidin-4-yl)-1H-pyrrolo[3,2- |
| | c]pyridin-3-yl)morpholine (Preparation 330) |
| | HPLC-C (29-59% MeCN). White solid (17.7 mg, 67%). LCMS m/z = |
| | 403.1 [M + H]⁺; ¹H NMR (400 MHz, CDCl₃) δ: 9.43 (s, 1H), 9.17 (s, 1H), |
| | 8.60 (s, 1H), 8.36 (br s, 1H), 7.62 (s, 1H), 7.14 (s, 1H), 3.97-3.94 (m, 4H), |
| | 3.20-3.17 (m, 4H), 2.27 (s, 3H), 2.05 (t, 3H). |
| 211 | N-(3-(azetidin-1-yl)-1-(6-(1,1-difluoroethyl)-2-methylpyrimidin-4-yl)-1H- |
| | pyrazolo[4,3-c]pyridin-6-yl)acetamide |
| | 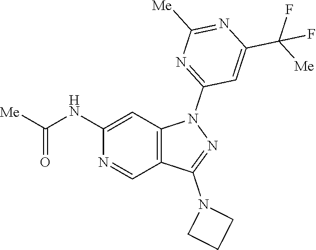 [SMILES: CC(=O)Nc1cc2c(cn1)c(N1CCC1)nn2-c1cc(C(C)(F)F)nc(C)n1] |
| | SM: 3-(azetidin-1-yl)-6-chloro-1-(6-(1,1-difluoroethyl)-2-methylpyrimidin- |
| | 4-yl)-1H-pyrazolo[4,3-c]pyridine (Preparation 331) |
| | HPLC-C (37-67% MeCN). White solid (68 mg, 80%). LCMS m/z = 388.1 |
| | [M + H]⁺; ¹H NMR (500 MHz, CDCl₃) δ: 9.54 (s, 1H), 8.51 (s, 1H), 8.38 (s, |
| | 1H), 7.80 (s, 1H), 4.33 (t, 4H), 2.84 (s, 3H), 2.61-2.55 (m, 2H), 2.28 (s, |
| | 3H), 1.99 (t, 3H). |
| 212 | N-(1-(6-(1,1-difluoroethyl)-2-methylpyrimidin-4-yl)-3-morpholino-1H- |
| | pyrazolo[4,3-c]pyridin-6-yl)acetamide |
| | 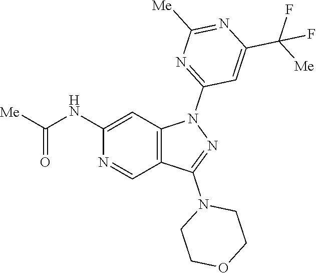 [SMILES: CC(=O)Nc1cc2c(cn1)c(N1CCOCC1)nn2-c1cc(C(C)(F)F)nc(C)n1] |
| | SM: 4-(6-chloro-1-(6-(1,1-difluoroethyl)-2-methylpyrimidin-4-yl)-1H- |
| | pyrazolo[4,3-c]pyridin-3-yl)morpholine (Preparation 332) |
| | HPLC-C (40-70% MeCN). White solid (42 mg, 40%). LCMS m/z = 418.1 |
| | [M + H]⁺; ¹H NMR (400 MHz, DMSO-d₆) δ: 10.81 (s, 1H), 9.54 (s, 1H), |
| | 9.10 (s, 1H), 7.70 (s, 1H), 3.85-3.77 (m, 4H), 3.62-3.57 (m, 4H), 2.72 (s, |
| | 3H), 2.15 (s, 3H), 2.00 (t, 3H). |
| 213 | N-(1-(2-(1,1-difluoroethyl)-6-methylpyrimidin-4-yl)-3-(4-methylpiperazin- |
| | 1-yl)-1H-pyrazolo[4,3-c]pyridin-6-yl)acetamide |
| | 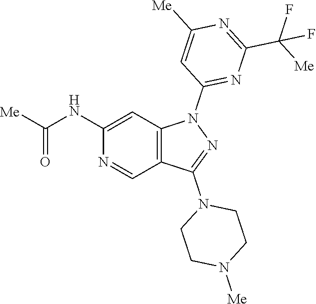 [SMILES: CC(=O)Nc1cc2c(cn1)c(N1CCN(C)CC1)nn2-c1cc(C)nc(C(C)(F)F)n1] |
| | SM: 6-chloro-1-(2-(1,1-difluoroethyl)-6-methylpyrimidin-4-yl)-3-(4- |
| | methylpiperazin-1-yl)-1H-pyrazolo[4,3-c]pyridine (Preparation 333) |
| | HPLC-C (35-65% MeCN). Yellow solid (26 mg, 29%). LCMS m/z = 431.1 |
| | [M + H]⁺; ¹H NMR (500 MHz, MeOH-d₄) δ: 9.33 (s, 1H), 8.86 (s, 1H), 7.66 |
| | (s, 1H), 3.71-3.62 (m, 4H), 2.74-2.67 (m, 4H), 2.60 (s, 3H), 2.41 (s, 3H), |
| | 2.28-2.16 (m, 6H). |
| 214 | (1-(2-(1,1-difluoroethyl)pyrimidin-4-yl)-3-((1R,4R)-5-methyl-2,5- |
| | diazabicyclo[2.2.2]octan-2-yl)-1H-pyrazolo[4,3-c]pyridin-6-yl)acetamide |
| | 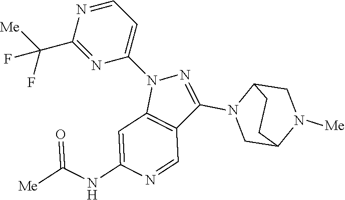 [SMILES: CC(=O)Nc1cc2c(cn1)c(N1CC3CCC1CN3C)nn2-c1ccnc(C(C)(F)F)n1] |
| | SM: 2-(6-chloro-1-(2-(1,1-difluoroethyl)pyrimidin-4-yl)-1H-pyrazolo[4,3- |
| | c]pyridin-3-yl)-5-methyl-2,5-diazabicyclo[2.2.2]octane (Preparation 335) |
| | HPLC-C (29-59% MeCN). White solid (12.1 mg, 23%). LCMS m/z = |
| | 443.2 [M + H]⁺; ¹H NMR (500 MHz, DMSO-d₆) δ: 10.76 (s, 1H), 9.38 (s, |
| | 1H), 8.97 (s, 1H), 8.82 (d, 1H), 7.77 (d, 1H), 4.27 (s, 1H), 4.13-4.11 (m, |
| | 1H), 3.72-3.69 (m, 1H), 2.94 (s, 2H), 2.84 (s, 1H), 2.36 (s, 3H), 2.22 (t, |
| | 3H), 2.15 (s, 3H), 2.05-2.00 (m, 2H), 1.82-1.79 (m, 1H), 1.69-1.65 (m, 1H). |
| 215 | N-(1-(2-(1,1-difluoroethyl)-6-methoxypyrimidin-4-yl)-3-(3- |
| | (dimethylamino)pyrrolidin-1-yl)-1H-pyrazolo[4,3-c]pyridin-6-yl)acetamide |
| | 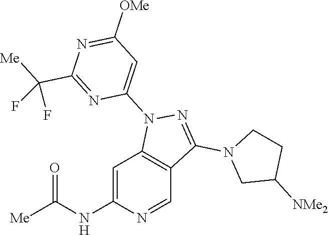 [SMILES: COc1cc(-n2nc(N3CCC(N(C)C)C3)c3cnc(NC(C)=O)cc32)nc(C(C)(F)F)n1] |
| | SM: 1-(6-chloro-1-(2-(1,1-difluoroethyl)-6-methoxypyrimidin-4-yl)-1H- |
| | pyrazolo[4,3-c]pyridin-3-yl)-N,N-dimethylpyrrolidin-3-amine (Preparation |
| | 363) |
| | HPLC-F (46-76% MeCN) White solid (8 mg, 22%). LCMS m/z = 461.3 |
| | [M + H]⁺; ¹H NMR (500 MHz, CDCl₃) δ: 9.45 (s, 1H), 8.70 (s, 1H), 8.05 (s, |
| | 1H), 7.13 (s, 1H), 4.08 (s, 3H), 3.96-3.92 (m, 2H), 3.75-3.72 (m, 1H), 3.55- |
| | 3.50 (m, 1H), 2.95-2.92 (m, 1H), 2.37 (s, 6H), 2.29-2.21 (m, 7H), 2.01-1.99 |
| | (m, 1H). |
| 216 | N-(1-(2-(1,1-difluoroethyl)-6-methylpyrimidin-4-yl)-3-(3- |
| | (dimethylamino)pyrrolidin-1-yl)-1H-pyrazolo[4,3-c]pyridin-6-yl)acetamide |
| | 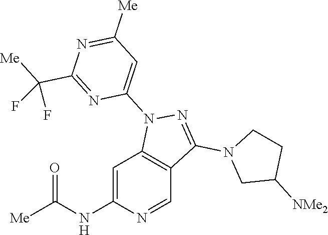 [SMILES: CC(=O)Nc1cc2c(cn1)c(N1CCC(N(C)C)C1)nn2-c1cc(C)nc(C(C)(F)F)n1] |
| | SM: 1-(6-chloro-1-(2-(1,1-difluoroethyl)-6-methylpyrimidin-4-yl)-1H- |
| | pyrazolo[4,3-c]pyridin-3-yl)-N,N-dimethylpyrrolidin-3-amine (Preparation |
| | 337) |
| | HPLC-F (39-69% MeCN) White solid (35 mg, 66%). LCMS m/z = 445.1 |
| | [M + H]⁺; ¹H NMR (500 MHz, CDCl₃) δ: 9.48 (s, 1H), 8.73 (s, 1H), 8.03 (s, |
| | 1H), 7.65 (s, 1H), 3.99-3.90 (m, 2H), 4.00-3.94 (m, 1H), 3.80-3.76 (m, 1H), |
| | 2.98-2.96 (m, 1H), 2.65 (s, 3H), 2.39 (s, 6H), 2.34-2.33 (m, 1H), 2.30-2.26 |
| | (m, 6H), 2.09-2.06 (m, 1H). |
| 217^{A} | N-(1-(4-(1,1-difluoroethyl)pyrimidin-2-yl)-3-(3-(dimethylamino)pyrrolidin- |
| | 1-yl)-1H-pyrazolo[4,3-c]pyridin-6-yl)acetamide |
| | 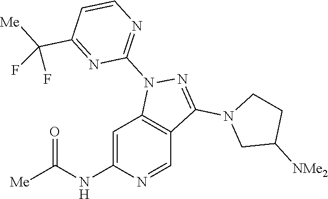 [SMILES: CC(=O)Nc1cc2c(cn1)c(N1CCC(N(C)C)C1)nn2-c1nccc(C(C)(F)F)n1] |
| | SM: 1-(6-chloro-1-(4-(1,1-difluoroethyl)pyrimidin-2-yl)-1H-pyrazolo[4,3- |
| | c]pyridin-3-y1)-N,N-dimethylpyrrolidin-3-amine (Preparation 338) |
| | HPLC-C (37-67% MeCN) White solid (15.6 mg, 16%). LCMS m/z = 431.1 |
| | [M + H]⁺; ¹H NMR (400 MHz, CDCl₃) δ: 9.38 (s, 1H), 8.96 (d, 2H), 8.76 (s, |
| | 1H), 8.15 (s, 1H), 7.36 (d, 1H), 4.09-4.00 (m, 2H), 3.84-3.81 (m, 1H), 3.64- |
| | 3.59 (m, 1H), 2.95-2.91 (m, 1H), 2.36-2.23 (m, 13H), 2.07-2.01 (m, 1H). |
| 218^{A} | N-(1-(2-(1,1-difluoroethyl)pyrimidin-4-yl)-3-(6-methyl-3,6- |
| | diazabicyclo[3.1.1]heptan-3-yl)-1H-pyrazolo[4,3-c]pyridin-6-yl)acetamide |
| | 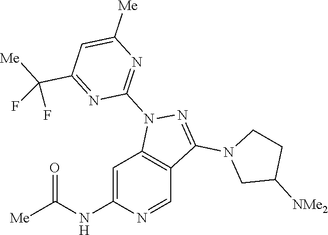 [SMILES: CC(=O)Nc1cc2c(cn1)c(N1CCC(N(C)C)C1)nn2-c1nc(C)cc(C(C)(F)F)n1] |
| | SM: 1-(6-chloro-1-(4-(1,1-difluoroethyl)-6-methylpyrimidin-2-yl)-1H- |
| | pyrazolo[4,3-c]pyridin-3-yl)-N,N-dimethylpyrrolidin-3-amine (Preparation |
| | 339) |
| | HPLC-C (27-57% MeCN) Yellow solid (9.5 mg, 18%). LCMS m/z = 445.1 |
| | [M + H]⁺; ¹H NMR (400 MHz, CDCl₃) δ: 9.38 (s, 1H), 8.75 (s, 1H), 8.16 (s, |
| | 1H), 7.24 (s, 1H), 4.11-4.00 (m, 2H), 3.82-3.80 (m, 1H), 3.61-3.57 (m, 1H), |
| | 2.93-2.91 (m, 1H), 2.71 (s, 3H), 2.36 (s, 6H), 2.31-2.20 (m, 7H), 2.04-2.02 |
| | (m, 1H). |
| 219 | N-(1-(2-(1,1-difluoroethyl)pyrimidin-4-yl)-3-(6-methyl-3,6- |
| | diazabicyclo[3.1.1]heptan-3-yl)-1H-pyrazolo[4,3-c]pyridin-6-yl)acetamide |
| | 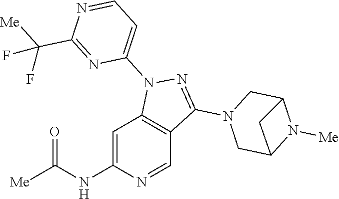 [SMILES: CC(=O)Nc1cc2c(cn1)c(N1CC3CC(C1)N3C)nn2-c1ccnc(C(C)(F)F)n1] |
| | SM: 3-(6-chloro-1-(2-(1,1-difluoroethyl)pyrimidin-4-yl)-1H-pyrazolo[4,3- |
| | c]pyridin-3-yl)-6-methyl-3,6-diazabicyclo[3.1.1 ]heptane (Preparation 340) |
| | HPLC-F (40-70% MeCN) Yellow solid (5 mg, 19%). LCMS m/z = 429.1 |
| | [M + H]⁺; ¹H NMR (400 MHz, CDCl₃) δ: 9.55 (s, 1H), 8.87 (d, 1H), 8.75 (d, |
| | 1H), 8.10 (s, 1H), 7.81 (d, 1H), 4.06 (d, 2H), 3.79-3.76 (m, 4H), 2.75-2.72 |
| | (m, 1H), 2.34-2.25 (m, 9H), 1.76-1.72 (m, 1H). |
| 220 | N-(1-(2-(1,1-difluoroethyl)pyrimidin-4-yl)-3-(6-oxa-1-azaspiro[3.3]heptan- |
| | 1-yl)-1H-pyrazolo[4,3-c]pyridin-6-yl)acetamide |
| | 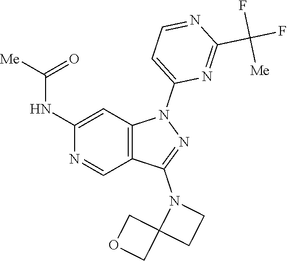 [SMILES: CC(=O)Nc1cc2c(cn1)c(N1CCC13COC3)nn2-c1ccnc(C(C)(F)F)n1] |
| | SM: 1-(6-chloro-1-(2-(1, 1-difluoroethyl)pyrimidin-4-yl)-1H-pyrazolo[4,3- |
| | c]pyridin-3-y1)-6-oxa-1-azaspiro[3.3]heptane (Preparation 341) |
| | HPLC-F (31-61% MeCN) Yellow solid (15 mg, 47%). LCMS m/z = 416.1 |
| | [M + H]⁺; ¹H NMR (500 MHz, CDCl₃) δ: 9.51 (s, 1H), 8.76-8.74 (m, 2H), |
| | 8.16 (s, 1H), 7.85 (d, 1H), 5.56 (d, 2H), 4.79 (d, 2H), 4.23 (t, 2H), 2.81 (t, |
| | 2H), 2.33-2.25 (m, 6H) |
| 221 | N-(1-(2-(1,1-difluoroethyl)pyrimidin-4-yl)-3-(3-methyl-3,6- |
| | diazabicyclo[3.2.0]heptan-6-yl)-1H-pyrazolo[4,3-c]pyridin-6-yl)acetamide |
| | 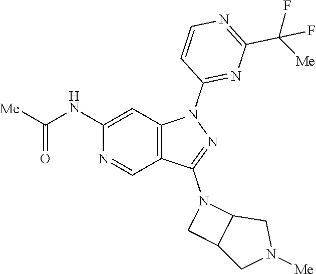 [SMILES: CC(=O)Nc1cc2c(cn1)c(N1CC3CN(C)CC31)nn2-c1ccnc(C(C)(F)F)n1] |
| | SM: 6-chloro-1-(2-(1,1-difluoroethyl)pyrimidin-4-yl)-3-(3-methyl-3,6- |
| | diazabicyclo[3.2.0]heptan-6-yl)-1H-pyrazolo[4,3-c]pyridine (Preparation |
| | 342) |
| | HPLC-F (45-75% MeCN) White solid (25.2 mg, 43%). LCMS m/z = 429.2 |
| | [M + H]⁺; ¹H NMR (500 MHz, CDCl₃) δ: 9.43 (s, 1H), 8.72 (d, 1H), 8.48 (s, |
| | 1H), 8.14 (s, 1H), 7.74 (d, 1H), 4.99-4.97 (m, 1H), 4.40-4.36 (m, 1H), 4.16- |
| | 4.13 (m, 1H), 3.53 (d, 1H), 3.34-3.31 (m, 1H), 3.17 (d, 1H), 2.49 (s, 3H), |
| | 2.32-2.24 (m, 6H), 2.20-2.14 (m, 1H), 2.13-2.11 (m, 1H). |
| 222 | N-(1-(2-(1,1-difluoroethyl)pyrimidin-4-yl)-3-(1- |
| | methylhexahydropyrrolo[3,4-b]pyrrol-5(1H)-yl)-1H-pyrazolo[4,3- |
| | c]pyridin-6-yl)acetamide |
| | 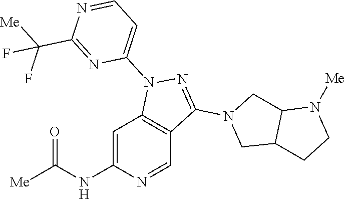 [SMILES: CC(=O)Nc1cc2c(cn1)c(N1CC3CCN(C)C3C1)nn2-c1ccnc(C(C)(F)F)n1] |
| | SM: 6-chloro-1-(2-(1,1-difluoroethyl)pyrimidin-4-yl)-3-(1- |
| | methylhexahydropyrrolo[3,4-b]pyrrol-5(1H)-yl)-1H-pyrazolo[4,3- |
| | c]pyridine (Preparation 343) |
| | HPLC-F (38-68% MeCN). White solid (5 mg, 32%). LCMS m/z = 443.3 |
| | [M + H]⁺; ¹H NMR (500 MHz, MeOH-d₄) δ: 9.38 (s, 1H), 8.90 (d, 1H), 8.73 |
| | (d, 1H), 7.85 (d, 1H), 3.93-3.86 (m, 2H), 3.68-3.65 (m, 2H), 3.14-3.12 (m, |
| | 3H), 2.45-2.44 (m, 4H), 2.27-2.20 (m, 7H), 1.82-1.78 (m, 1H). |
| 223 | N-(1-(2-(1,1-difluoroethyl)pyrimidin-4-yl)-3-(3-(dimethylamino)-3- |
| | methylpyrrolidin-1-yl)-1H-pyrazolo[4,3-c]pyridin-6-yl)acetamide |
| | 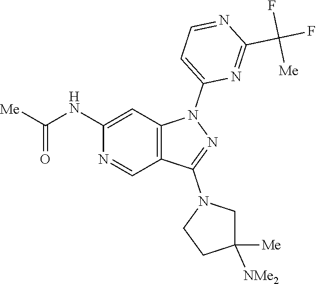 [SMILES: CC(=O)Nc1cc2c(cn1)c(N1CCC(C)(N(C)C)C1)nn2-c1ccnc(C(C)(F)F)n1] |
| | SM: 1-(6-chloro-1-(2-(1,1-difluoroethyl)pyrimidin-4-yl)-1H-pyrazolo[4,3- |
| | c]pyridin-3-yl)-N,N,3-trimethylpyrrolidin-3-amine (Preparation 344) |
| | HPLC-F (39-69% MeCN) White solid (15 mg, 47%). LCMS m/z = 445.2 |
| | [M + H]⁺; ¹H NMR (400 MHz, MeOH-d₄) δ: 9.36 (s, 1H), 8.87 (s, 1H), 8.72 |
| | (d, 1H), 7.84 (d, 1H), 3.96-3.89 (m, 1H), 3.86-3.85 (m, 1H), 3.78-3.76 (m, |
| | 1H), 3.65-3.63 (m, 1H), 2.42 (s, 6H), 2.29-2.24 (m, 6H), 2.19-2.12 (m, 2H), |
| | 1.25 (s, 3H). |
| 224^{A} | N-(1-(2-(1,1-difluoroethyl)pyrimidin-4-yl)-3-(5- |
| | methylhexahydropyrrolo[3,4-c]pyrrol-2(1H)-yl)-1H-pyrazolo[4,3- |
| | c]pyridin-6-yl)acetamide |
| | 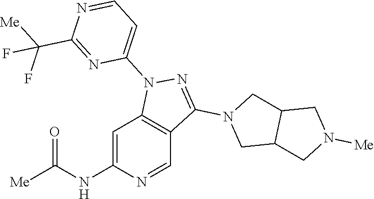 [SMILES: CC(=O)Nc1cc2c(cn1)c(N1CC3CN(C)CC3C1)nn2-c1ccnc(C(C)(F)F)n1] |
| | SM: 6-chloro-1-(2-(1,1-difluoroethyl)pyrimidin-4-yl)-3-(5- |
| | methylhexahydropyrrolo[3,4-c]pyrrol-2(1H)-yl)-1H-pyrazolo[4,3- |
| | c]pyridine (Preparation 345) |
| | HPLC-C (33-63% MeCN). White solid (31.3 mg, 38%). LCMS m/z = |
| | 443.2 [M + H]⁺; ¹H NMR (500 MHz, MeOH-d₄) δ: 9.36 (s, 1H), 8.88 (s, |
| | 1H), 8.73 (d, 1H), 7.80 (d, 1H), 3.83-3.77 (m, 2H), 3.69-3.64 (m, 2H), 3.12- |
| | 3.11 (m, 2H), 2.92-2.88 (m, 2H), 2.61-2.57 (m, 2H), 2.39 (s, 3H), 2.27-2.19 |
| | (m, 6H). |
| 225^{A} | N-(1-(2-(1,1-difluoroethyl)pyrimidin-4-yl)-3-(8-methyl-3,8- |
| | diazabicyclo[3.2.1]octan-3-yl)-1H-pyrazolo[4,3-c]pyridin-6-yl)acetamide |
| | 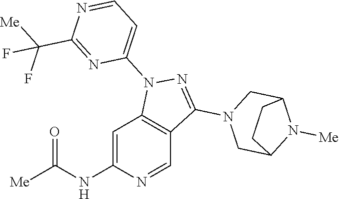 [SMILES: CC(=O)Nc1cc2c(cn1)c(N1CC3CCC(C1)N3C)nn2-c1ccnc(C(C)(F)F)n1] |
| | SM: 6-chloro-1-(2-(1,1-difluoroethyl)pyrimidin-4-yl)-3-(8-methyl-3,8- |
| | diazabicyclo[3.2.1]octan-3-yl)-1H-pyrazolo[4,3-c]pyridine (Preparation |
| | 346) |
| | HPLC-C (31-61% MeCN). White solid (12.8 mg, 14%). LCMS m/z = |
| | 443.2 [M + H]⁺; ¹H NMR (500 MHz, CDCl₃) δ: 9.51 (s, 1H), 8.75-8.73 (m, |
| | 2H), 8.14 (s, 1H), 7.76 (d, 1H), 3.81-3.79 (m, 2H), 3.48-3.46 (m, 2H), 3.33- |
| | 3.32 (m, 2H), 2.39 (s, 3H), 2.32-2.24 (m, 6H), 2.13-2.12 (m, 2H), 1.86-1.85 |
| | (m, 2H). |
| 226^{A} | N-(1-(2-(1,1-difluoroethyl)pyrimidin-4-yl)-3-(3-methyl-3,8- |
| | diazabicyclo[3.2.1]octan-8-yl)-1H-pyrazolo[4,3-c]pyridin-6-yl)acetamide |
| | 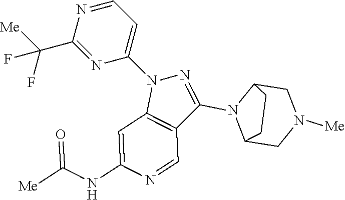 [SMILES: CC(=O)Nc1cc2c(cn1)c(N1C3CCC1CN(C)C3)nn2-c1ccnc(C(C)(F)F)n1] |
| | SM: 6-chloro-1-(2-(1,1-difluoroethyl)pyrimidin-4-yl)-3-(3-methyl-3,8- |
| | diazabicyclo[3.2.1]octan-8-yl)-1H-pyrazolo[4,3-c]pyridine (Preparation |
| | 347) |
| | HPLC-C (25-55% MeCN). |
| | White solid (19 mg, 22%). LCMS m/z = 443.2 [M + H]⁺; ¹H NMR (500 |
| | MHz, MeOH-d₄) δ: 9.44 (s, 1H), 8.91 (s, 1H), 8.77 (d, 1H), 7.89 (d, 1H), |
| | 2.86-2.84 (m, 2H), 2.67-2.65 (m, 2H), 2.28-2.24 (m, 9H), 2.24-2.22 (m, |
| | 4H), 2.11-2.03 (m, 2H). |
| 227^{A} | N-(1-(2-(1, 1-difluoroethyl)pyrimidin-4-y1)-3-(5-methyl-2,5- |
| | diazabicyclo[2.2.1]heptan-2-yl)-1H-pyrazolo[4,3-c]pyridin-6-yl)acetamide |
| | 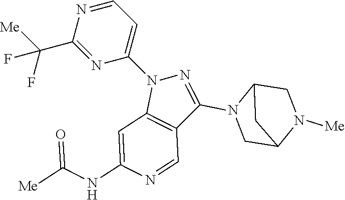 [SMILES: CC(=O)Nc1cc2c(cn1)c(N1CC3CC1CN3C)nn2-c1ccnc(C(C)(F)F)n1] |
| | SM: 6-chloro-1-(2-(1,1-difluoroethyl)pyrimidin-4-yl)-3-(5-methyl-2,5- |
| | diazabicyclo[2.2.1]heptan-2-yl)-1H-pyrazolo[4,3-c]pyridine (Preparation |
| | 348) |
| | HPLC-F (34-64% MeCN) |
| | White solid (11.3 mg, 21%). LCMS m/z = 429.1 [M + H]⁺; ¹H NMR (500 |
| | MHz, CDCl₃) δ: 9.49 (s, 1H), 8.73 (d, 1H), 8.68 (s, 1H), 8.09 (s, 1H), 7.77 |
| | (d, 1H), 4.62 (s, 1H), 3.89-3.73 (m, 1H), 3.72-3.70 (m, 1H), 3.59 (s, 1H), |
| | 3.00-2.98 (m, 1H), 2.89-2.87 (m, 1H), 2.44 (s, 3H), 2.33-2.27 (m, 6H), |
| | 2.25-2.06 (m, 1H), 1.97-1.96 (m, 1H). |
| 228^{A} | N-(3-(3,3-difluoro-1-oxa-6-azaspiro[3.3]heptan-6-yl)-1-(2-(1,1- |
| | difluoroethyl)pyrimidin-4-yl)-1H-pyrazolo[4,3-c]pyridin-6-yl)acetamide |
| | 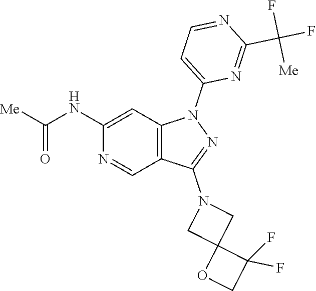 [SMILES: CC(=O)Nc1cc2c(cn1)c(N1CC3(C1)OCC3(F)F)nn2-c1ccnc(C(C)(F)F)n1] |
| | SM: 6-(6-chloro-1-(2-(1,1-difluoroethyl)pyrimidin-4-yl)-1H-pyrazolo[4,3- |
| | c]pyridin-3-yl)-3,3-difluoro-1-oxa-6-azaspiro[3.3 ]heptane (Preparation 349) |
| | HPLC-F (41-71% MeCN) |
| | White solid (8.5 mg, 40%). LCMS m/z = 452.0 [M + H]⁺; ¹H NMR (400 |
| | MHz, MeOH-d₄) δ: 8.70 (d, 1H), 8.39 (s, 1H), 7.77 (d, 1H), 7.66 (s, 1H), |
| | 4.76-4.73 (m, 2H), 4.61 (s, 2H) 4.38-4.34 (m, 2H) 2.29-2.03 (m, 6H). |

A-Pd₂(dba)₃/Xantphos was used instead of Brettphos Pd G3

### Example 229: N-(1-(4-(1,1-difluoroethyl)pyrimidin-2-yl)-3-morpholino-1H-pyrazolo[4,3-c]pyridin-6-yl)cyclopropanecarboxamide

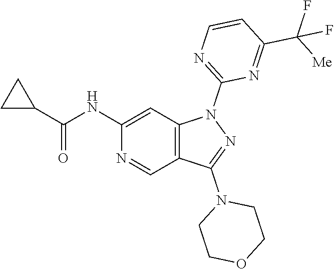 [SMILES: CC(F)(F)c1ccnc(-n2nc(N3CCOCC3)c3cnc(NC(=O)C4CC4)cc32)n1]

A mixture of 4-(6-chloro-1-(4-(1,1-difluoroethyl)pyrimidin-2-yl)-1H-pyrazolo[4,3-c]pyridin-3-yl)morpholine (Preparation 334, 100 mg, 0.263 mmol), cyclopropylacetamide (26.8 mg, 0.315 mmol), BrettPhos Pd G3 (47.6 mg, 0.053 mmol) and Cs₂CO₃ (171.1 mg, 0.525 mmol) in dioxane (5 mL) was stirred at 100° C. for 2 h under N₂. The cooled mixture was concentrated and purified by HPLC-F (46-76% MeCN) to give N-(1-(4-(1,1-difluoroethyl)pyrimidin-2-yl)-3-morpholino-1H-pyrazolo[4,3-c]pyridin-6-yl)cyclopropanecarboxamide (32.3 mg, 28.6% yield) as a yellow solid. LCMS m/z=430.1 [M+H]⁺; ¹H NMR (500 MHz, CDCl₃) δ: 9.43 (s, 1H), 8.96-8.83 (m, 2H), 8.36 (br s, 1H), 7.41 (s, 1H), 3.95-3.94 (m, 4H), 3.68-3.67 (m, 4H), 2.24 (t, 3H), 1.60-1.56 (m, 1H), 1.15-1.14 (m, 2H), 0.97-0.88 (m, 2H).

### Example 230: N-(1-(4-(1,1-difluoroethyl)pyrimidin-2-yl)-3-morpholino-1H-pyrazolo[4,3-c]pyridin-6-yl)-3-methoxypropanamide

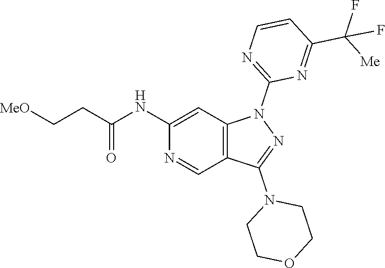 [SMILES: COCCC(=O)Nc1cc2c(cn1)c(N1CCOCC1)nn2-c1nccc(C(C)(F)F)n1]

N-(1-(4-(1,1-difluoroethyl)pyrimidin-2-yl)-3-morpholino-1H-pyrazolo[4,3-c]pyridin-6-yl)-3-methoxypropanamide was obtained as a white solid, 9.2 mg, 10% yield, from 4-(6-chloro-1-(4-(1,1-difluoroethyl)pyrimidin-2-yl)-1H-pyrazolo[4,3-c]pyridin-3-yl)morpholine (Preparation 334) and 3-methoxypropanamide, following a similar procedure to that described in Example 229. LCMS m/z=448.2 [M+H]⁺; ¹H NMR (500 MHz, CDCl₃) δ: 9.86 (br s, 1H), 9.58 (s, 1H), 9.00 (d, 1H), 8.80 (s, 1H), 7.47 (d, 1H), 3.95-3.93 (m, 4H), 3.81-3.78 (m, 2H), 3.68-3.67 (m, 4H), 3.47 (s, 3H), 2.79-2.76 (m, 2H), 2.27 (t, 3H).

### Example 231: N-(1-(4-(1,1-difluoroethyl)pyrimidin-2-yl)-3-morpholino-1H-pyrazolo[4,3-c]pyridin-6-yl)-2-fluorocyclopropane-1-carboxamide

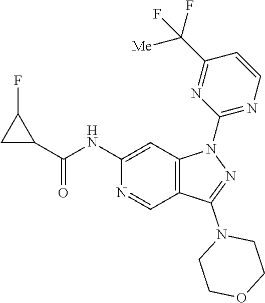 [SMILES: CC(F)(F)c1ccnc(-n2nc(N3CCOCC3)c3cnc(NC(=O)C4CC4F)cc32)n1]

N-(1-(4-(1,1-Difluoroethyl)pyrimidin-2-yl)-3-morpholino-1H-pyrazolo[4,3-c]pyridin-6-yl)-2-fluorocyclopropane-1-carboxamide was obtained as a white solid, 9.0 mg, 7.7% yield, from 4-(6-chloro-1-(4-(1,1-difluoroethyl)pyrimidin-2-yl)-1H-pyrazolo[4,3-c]pyridin-3-yl)morpholine (Preparation 334) and 2-fluorocyclopropane-1-carboxamide, following a similar procedure to that described in Example 229. LCMS m/z=448.1 [M+H]⁺; ¹H NMR (500 MHz, CDCl₃) δ: 9.42 (s, 1H), 8.96 (d, 1H), 8.78 (s, 1H), 8.30 (s, 1H), 7.41 (d, 1H), 4.91-4.75 (m, 1H), 3.96-3.93 (m, 4H), 3.68-3.66 (m, 4H), 2.27 (t, 3H), 1.96-1.87 (m, 2H), 1.27-1.24 (m, 1H).

### Example 232 and 233: N-(1-(4-(1,1-difluoroethyl)-6-methoxypyrimidin-2-yl)-3-(3-(dimethylamino)pyrrolidin-1-yl)-1H-pyrazolo[4,3-c]pyridin-6-yl)acetamide and N-(1-(6-(1,1-difluoroethyl)-2-methoxypyrimidin-4-yl)-3-(3-(dimethylamino)pyrrolidin-1-yl)-1H-pyrazolo[4,3-c]pyridin-6-yl)acetamide

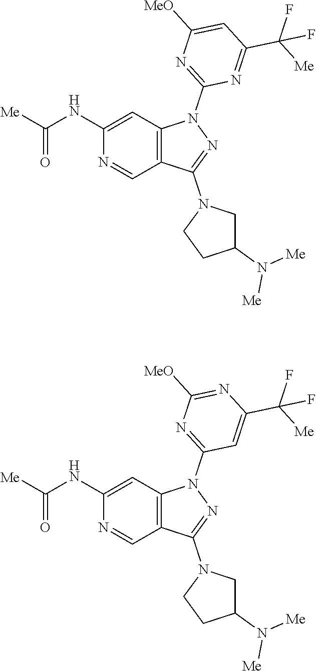 [SMILES: COc1cc(C(C)(F)F)nc(-n2nc(N3CCC(N(C)C)C3)c3cnc(NC(C)=O)cc32)n1 ; COc1nc(-n2nc(N3CCC(N(C)C)C3)c3cnc(NC(C)=O)cc32)cc(C(C)(F)F)n1]

To a solution of 1-(6-chloro-1-(4-(1,1-difluoroethyl)-6-methoxypyrimidin-2-yl)-1H-pyrazolo[4,3-c]pyridin-3-yl)-N,N-dimethylpyrrolidin-3-amine and 1-(6-chloro-1-(6-(1,1-difluoroethyl)-2-methoxypyrimidin-4-yl)-1H-pyrazolo[4,3-c]pyridin-3-yl)-N,N-dimethylpyrrolidin-3-amine (Preparation 350, 80 mg, 0.183 mmol) in dioxane (3 mL) was added acetamide (13.0 mg, 0.220 mmol), Cs₂CO₃ (119.3 mg, 0.366 mmol) and Brettphos Pd G3 (16.6 mg, 0.018 mmol) and the mixture was stirred at 100° C. for 16 h under N₂. The mixture was concentrated and was purified by Prep-HPLC-L (gradient: 35 to 63%) to give N-(1-(4-(1,1-difluoroethyl)-6-methoxypyrimidin-2-yl)-3-(3-(dimethylamino)pyrrolidin-1-yl)-1H-pyrazolo[4,3-c]pyridin-6-yl)acetamide (4.4 mg, 5.22% yield) as a yellow solid. LCMS m/z=461.1 [M+H]f¹H NMR: (400 MHz, CDCl₃) δ: ppm 9.38 (s, 1H), 8.74 (s, 1H), 8.03 (s, 1H), 6.78 (s, 1H), 4.26 (s, 3H), 4.07-3.98 (m, 2H), 3.82-3.78 (m, 1H), 3.61-3.57 (m, 1H), 2.95-2.93 (m, 1H), 2.37 (s, 6H), 2.29-2.20 (m, 4H), 2.15 (t, J=18.8 Hz, 3H), 2.05-2.03 (m, 1H).

And N-(1-(6-(1,1-difluoroethyl)-2-methoxypyrimidin-4-yl)-3-(3-(dimethylamino)pyrrolidin-1-yl)-1H-pyrazolo[4,3-c]pyridin-6-yl)acetamide (4 mg, 4.74% yield) as a yellow solid. LCMS m/z=461.1 [M+H]⁺. ¹H NMR: (400 MHz, CDCl₃) δ: ppm 9.49 (s, 1H), 8.73 (s, 1H), 8.03 (s, 1H), 7.67 (s, 1H), 4.29 (s, 3H), 3.99-3.93 (m, 2H), 3.80-3.75 (m, 1H), 3.57-3.53 (m, 1H), 2.98-2.95 (m, 1H), 2.38 (s, 6H), 2.32-2.25 (m, 4H), 2.07-1.96 (m, 4H).

### Example 234: N-(3-(2-cyanocyclopropyl)-1-(2-(1,1-difluoroethyl)pyrimidin-4-yl)-1H-pyrazolo[4,3-c]pyridin-6-yl)acetamide

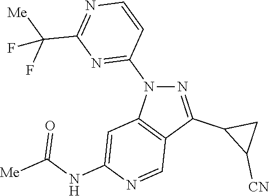 [SMILES: CC(=O)Nc1cc2c(cn1)c(C1CC1C#N)nn2-c1ccnc(C(C)(F)F)n1]

To a solution of 2-(4,4,5,5-tetramethyl-1,3,2-dioxaborolan-2-yl)cyclopropane-1-carbonitrile (Preparation 375, 244 mg, 1.26 mmol) in dioxane (2 mL) and H₂O (0.2 mL) was added N-(3-bromo-1-(2-(1,1-difluoroethyl)pyrimidin-4-yl)-1H-pyrazolo[4,3-c]pyridin-6-yl)acetamide (Preparation 368, 100 mg, 0.252 mmol), K₂CO₃ (105 mg, 0.757 mmol) and Pd(dppf)Cl₂ (18.5 mg, 0.025 mmol) and the mixture stirred at 100° C. for 1 h under N₂. The mixture was concentrated and purified by HPLC-C (29-59% MeCN) to give N-(3-(2-cyanocyclopropyl)-1-(2-(1,1-difluoroethyl)pyrimidin-4-yl)-1H-pyrazolo[4,3-c]pyridin-6-yl)acetamide as a white solid (29.5 mg, 24%). LCMS m/z=384.1 [M+H]⁺. ¹H NMR (400 MHz, CDCl₃) δ: 9.55 (s, 1H), 8.86-8.44 (m, 2H), 8.18 (s, 1H), 7.85 (d, 1H), 2.98-2.96 (m, 1H), 2.34-2.24 (m, 7H), 2.19 (s, 1H), 1.89-1.84 (m, 2H).

### Example 235 N-(3-(3-cyano-3-fluoropyrrolidin-1-yl)-1-(4-(1,1-difluoroethyl) pyrimidin-2-yl)-1H-pyrazolo[4,3-c]pyridin-6-yl)acetamide

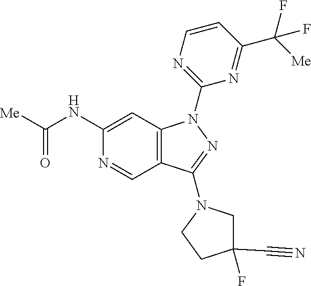 [SMILES: CC(=O)Nc1cc2c(cn1)c(N1CCC(F)(C#N)C1)nn2-c1nccc(C(C)(F)F)n1]

To a solution of N-(3-bromo-1-(4-(1,1-difluoroethyl)pyrimidin-2-yl)-1H-pyrazolo[4,3-c]pyridin-6-yl)acetamide (Preparation 367, 90 mg, 0.227 mmol) in DMSO (3 mL) was added 3-fluoropyrrolidine-3-carbonitrile hydrochloride (129.3 mg, 0.859 mmol), (1,2-dimethoxyethane)nickel dibromide (3.5 mg, 0.011 mmol), Ru(bpy)₃(PF6)₂ (2.0 mg, 0.002 mmol) and DABCO (50.8 mg, 0.453 mmol) and the mixture irradiated (kessil 450 nM lamp) with heating at 80° C. for 16 h under N₂. The mixture was diluted with water (5 mL), extracted with EtOAc (3×10 mL). The combined organics were washed with brine (3×20 mL), dried (Na₂SO₄) and evaporated to dryness in vacuo. The residue was purified by prep-TLC (75% EtOAc/PE) followed by prep-HPLC-C (30-60% MeCN) to afford N-(3-(3-cyano-3-fluoropyrrolidin-1-yl)-1-(4-(1,1-difluoroethyl)pyrimidin-2-yl)-1H-pyrazolo[4,3-c]pyridin-6-yl)acetamide as a white solid (5.1 mg, 5%). LCMS m/z=431.0 [M+H]. 1HNMR: (400 MHz, CDCl₃) δ ppm: 9.41 (s, 1H), 8.97 (d, J=5.2 Hz, 1H), 8.74 (s, 1H), 8.11 (s, 1H), 7.42 (d, J=5.2 Hz, 1H), 4.46-4.27 (m, 2H), 4.19-4.18 (m, 1H), 4.08-4.06 (m, 1H), 2.88-2.65 (m, 2H), 2.28 (t, J=19.2 Hz, 3H), 2.27 (s, 3H)

Example 236-247: The title compounds were prepared from the appropriate bromide (RBr) and the appropriate amine using an analogous method to that described for Example 235. RBr-1: N-(3-bromo-1-(4-(1,1-difluoroethyl)pyrimidin-2-yl)-1H-pyrazolo[4,3-c]pyridin-6-yl)acetamide (Preparation 367); RBr-2: N-(3-bromo-1-(2-(1,1-difluoroethyl)pyrimidin-4-yl)-1H-pyrazolo[4,3-c]pyridin-6-yl)acetamide (Preparation 368)

| Example No | Name/Structure/RBr/Amine/Data |
|---|---|
| 236 | N-(1-(4-(1,1-difluoroethyl)pyrimidin-2-yl)-3-(3-(difluoromethoxy)azetidin- |
| | 1-yl)-1H-pyrazolo[4,3-c]pyridin-6-yl)acetamide |
| | 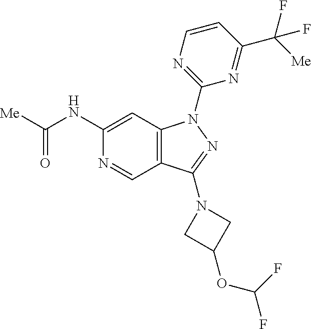 [SMILES: CC(=O)Nc1cc2c(cn1)c(N1CC(OC(F)F)C1)nn2-c1nccc(C(C)(F)F)n1] |
| | RBr-1; Amine: 3-(difluoromethoxy)azetidine hydrochloride |
| | HPLC-C (30-50% MeCN). |
| | White solid (25 mg, 37%). LCMS m/z = 440.0 [M + H]⁺. ¹H NMR (500 MHz, |
| | CDCl₃) δ: 9.42 (s, 1H), 8.97 (d, 1H), 8.57-8.53 (m, 2H), 7.43 (d, 1H), |
| | 6.30 (t, 1H), 5.27-5.23 (m, 1H), 4.66-4.50 (m, 2H), 4.44-4.41 (m, 2H), 2.31- |
| | 2.23 (m, 6H). |
| 237 | N-(3-(azetidin-1-yl)-1-(2-(1,1-difluoroethyl)pyrimidin-4-yl)-1H- |
| | pyrazolo[4,3-c]pyridin-6-yl)acetamide |
| | 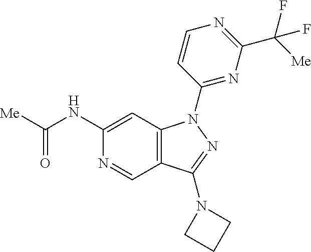 [SMILES: CC(=O)Nc1cc2c(cn1)c(N1CCC1)nn2-c1ccnc(C(C)(F)F)n1] |
| | RBr-2; Amine: azetidine hydrochloride |
| | HPLC-C (38-68% MeCN). White solid (13.4 mg, 18%). LCMS m/z = |
| | 374.1 [M + H]⁺. ¹H NMR (500 MHz, CDCl₃) δ: 9.50 (s, 1H), 8.76 (d, 1H), |
| | 8.52 (s, 1H), 8.48 (s, 1H), 7.76 (d, 1H), 4.35-4.33 (m, 4H), 2.63-2.57 (m, |
| | 2H), 2.32-2.24 (m, 6H). |
| 238 | N-(1-(2-(1, 1-difluoroethyl)pyrimidin-4-yl)-3-(2-methylazetidin-1-yl)-1H- |
| | pyrazolo[4,3-c]pyridin-6-yl)acetamide |
| | 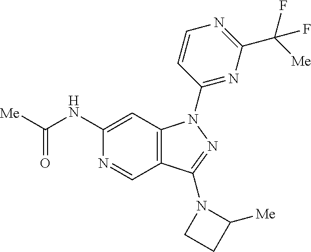 [SMILES: CC(=O)Nc1cc2c(cn1)c(N1CCC1C)nn2-c1ccnc(C(C)(F)F)n1] |
| | RBr-2; Amine: 2-methylazetidine hydrochloride |
| | HPLC-F (45-75% MeCN). White solid (9.8 mg, 16%). LCMS m/z = 388.1 |
| | [M + H]⁺. ¹H NMR (500 MHz, MeOH-d₄) δ: 9.37 (s, 1H), 8.74 (d, 1H), 8.67 |
| | (d, 1H), 7.83 (d, 1H), 4.67-4.65 (m, 1H), 4.37-4.34 (m, 1H), 4.20-4.17 (m, |
| | 1H), 2.64-2.61 (m, 1H), 2.28-2.19 (m, 7H), 1.60 (d, 3H). |
| 239 | N-(1-(2-(1, 1-difluoroethyl)pyrimidin-4-yl)-3-(3-(difluoromethoxy)azetidin- |
| | 1-yl)-1H-pyrazolo[4,3-c]pyridin-6-yl)acetamide |
| | 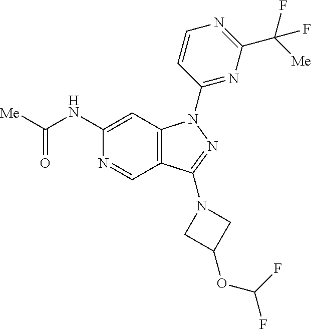 [SMILES: CC(=O)Nc1cc2c(cn1)c(N1CC(OC(F)F)C1)nn2-c1ccnc(C(C)(F)F)n1] |
| | RBr-2; Amine: 3-(difluoromethoxy)azetidine hydrochloride |
| | HPLC-C (33-53% MeCN). White solid (10 mg, 15%). LCMS m/z = 440.0 |
| | [M + H]⁺. ¹H NMR (500 MHz, DMSO-d₆) δ: 10.78 (s, 1H), 9.35 (s, 1H), |
| | 8.86 (d, 1H), 8.79 (s, 1H), 7.77 (d, 1H), 6.83 (t, 1H), 5.24-5.19 (m, 1H), |
| | 4.66-4.63 (m, 2H), 4.31-4.28 (m, 2H), 2.22 (t, 3H), 2.16 (s, 3H). |
| 240 | N-(3-(2-oxa-5-azabicyclo[2.2.1]heptan-5-yl)-1-(2-(1,1- |
| | difluoroethyl)pyrimidin-4-yl)-1H-pyrazolo[4,3-c]pyridin-6-yl)acetamide |
| | 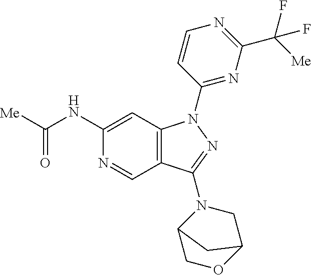 [SMILES: CC(=O)Nc1cc2c(cn1)c(N1CC3CC1CO3)nn2-c1ccnc(C(C)(F)F)n1] |
| | RBr-2; Amine: 2-oxa-5-azabicyclo[2.2.1 ]heptane hydrochloride |
| | HPLC-C (38-68% MeCN). White solid (15.1 mg, 18%). LCMS m/z = |
| | 416.1 [M + H]⁺. ¹H NMR (400 MHz, CDCl₃) δ: 9.50 (s, 1H), 8.75 (d, 1H), |
| | 8.66 (s, 1H), 8.08 (s, 1H), 7.77 (d, 1H), 4.81-4.77 (m, 2H), 4.13-4.11 (m, |
| | 1H), 3.95-3.87 (m, 2H), 3.75-3.73 (m, 1H), 2.34-2.24 (m, 6H), 2.11-2.04 |
| | (m, 2H). |
| 241 | N-(3-(8-oxa-3-azabicyclo[3.2.1]octan-3-yl)-1-(2-(1,1- |
| | difluoroethyl)pyrimidin-4-yl)-1H-pyrazolo[4,3-c]pyridin-6-yl)acetamide |
| | 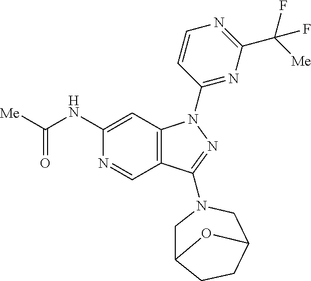 [SMILES: CC(=O)Nc1cc2c(cn1)c(N1CC3CCC(C1)O3)nn2-c1ccnc(C(C)(F)F)n1] |
| | RBr-2; Amine: 8-oxa-3-azabicyclo[3.2.1 ]octane hydrochloride |
| | HPLC-I (35-55% MeCN). White solid (11.3 mg, 10.5%). LCMS m/z = |
| | 430.3 [M + H]⁺. ¹H NMR (500 MHz, DMSO-d₆) δ: 10.80 (s, 1H), 9.41 (s, |
| | 1H), 9.07 (s, 1H), 8.86 (d, 1H), 7.81 (d, 1H), 4.49 (s, 2H), 3.88-3.85 (m, |
| | 2H), 3.34-3.31 (m, 2H), 2.22 (t, 3H), 2.15 (s, 3H), 1.97-1.93 (m, 4H). |
| 242 | N-(3-(6-oxa-3-azabicyclo[3.1.1]heptan-3-yl)-1-(2-(1,1- |
| | difluoroethyl)pyrimidin-4-yl)-1H-pyrazolo[4,3-c]pyridin-6-yl)acetamide |
| | 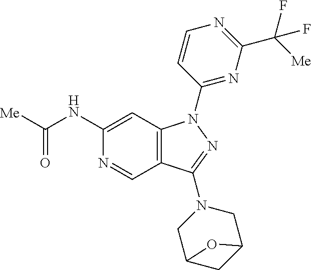 [SMILES: CC(=O)Nc1cc2c(cn1)c(N1CC3CC(C1)O3)nn2-c1ccnc(C(C)(F)F)n1] |
| | RBr-2; Amine: 6-oxa-3-azabicyclo[3.1.1 ]heptane hydrochloride |
| | HPLC-C (27-57% MeCN). White solid (9 mg, 9%). LCMS m/z = 416.1 |
| | [M + H]⁺. ¹H NMR (400 MHz, CDCl₃) δ: 9.55 (s, 1H), 8.83 (s, 1H), 8.75 (d, |
| | 1H), 8.07 (s, 1H), 7.81 (d, 1H), 4.84 (d, 2H), 4.10 (d, 2H), 3.99 (d, 2H), |
| | 3.43-3.36 (m, 1H), 2.35-2.25 (m, 6H), 2.16-2.13 (m, 1H). |
| 243 | N-(1-(4-(1, 1-difluoroethyl)pyrimidin-2-yl)-3-(3- |
| | (difluoromethoxy)pyrrolidin-1-yl)-1H-pyrazolo[4,3-c]pyridin-6- |
| | yl)acetamide |
| | 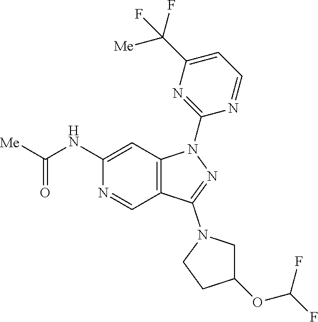 [SMILES: CC(=O)Nc1cc2c(cn1)c(N1CCC(OC(F)F)C1)nn2-c1nccc(C(C)(F)F)n1] |
| | RBr-1; Amine: 3-(difluoromethoxy)pyrrolidine |
| | HPLC-C (32-62% MeCN). Yellow solid (15.1 mg, 15%). LCMS m/z = |
| | 454.0 [M + H]⁺. ¹H NMR (400 MHz, CDCl₃) δ: 9.38 (s, 1H), 8.96 (d, 1H), |
| | 8.77 (s, 1H), 8.12 (s, 1H), 7.37 (d, 1H), 6.29 (t, 1H), 5.07 (s, 1H), 3.96-4.08 |
| | (m, 4H), 2.33-2.30 (m, 2H), 2.23-2.28 (m, 6H). |
| 244 | N-(1-(2-(1, 1-difluoroethyl)pyrimidin-4-yl)-3-(2-methylpyrrolidin-1-yl)-1H- |
| | pyrazolo[4,3-c]pyridin-6-yl)acetamide |
| | 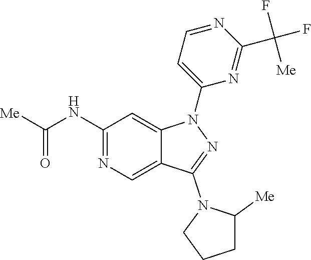 [SMILES: CC(=O)Nc1cc2c(cn1)c(N1CCCC1C)nn2-c1ccnc(C(C)(F)F)n1] |
| | RBr-2; Amine: 2-methylpyrrolidine hydrochloride |
| | HPLC-F (48-78% MeCN) |
| | White solid (4.8 mg, 4.8%). LCMS m/z = 402.1 [M + H]⁺. ¹H NMR (500 |
| | MHz, CDCl₃) δ: 9.50 (s, 1H), 8.74-8.72 (m, 2H), 8.12 (s, 1H), 7.78 (d, 1H), |
| | 4.33-4.29 (m, 1H), 3.90-3.87 (m, 1H), 3.70-3.68 (m, 1H), 2.29 (t, 3H), 2.28 |
| | (s, 3H), 2.20-2.18 (m, 2H), 2.18-2.07 (m, 1H), 1.82-1.79 (m, 1H), 1.37 (d, |
| | 3H). |
| 245 | N-(3-(3,3-difluoro-4-hydroxypyrrolidin-1-yl)-1-(2-(1,1- |
| | difluoroethyl)pyrimidin-4-yl)-1H-pyrazolo[4,3-c]pyridin-6-yl)acetamide |
| | 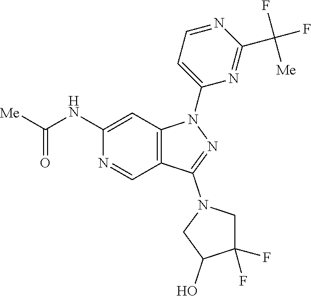 [SMILES: CC(=O)Nc1cc2c(cn1)c(N1CC(O)C(F)(F)C1)nn2-c1ccnc(C(C)(F)F)n1] |
| | RBr-2; Amine: 4,4-difluoropyrrolidin-3-ol |
| | HPLC-C (27-57% MeCN). White solid (7 mg, 6%). LCMS m/z = 440.0 |
| | [M + H]⁺. ¹H NMR (500 MHz, DMSO-d₆) δ: 10.79 (s, 1H), 9.38 (s, 1H), |
| | 8.99 (s, 1H), 8.87 (d, 1H), 7.82 (d, 1H), 6.27 (d, 1H), 4.43 (s, 1H), 4.19- |
| | 4.08 (m, 3H), 3.75-3.72 (m, 1H), 2.23 (t, 3H), 2.16 (s, 3H). |
| 246 | N-(3-(3-cyano-3-cyclopropylpyrrolidin-1-yl)-1-(2-(1,1- |
| | difluoroethyl)pyrimidin-4-yl)-1H-pyrazolo[4,3-c]pyridin-6-yl)acetamide |
| | 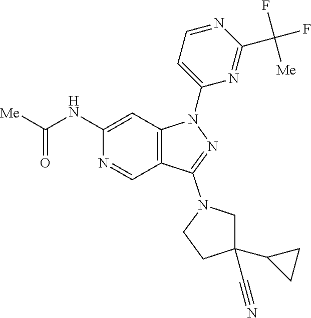 [SMILES: CC(=O)Nc1cc2c(cn1)c(N1CCC(C#N)(C3CC3)C1)nn2-c1ccnc(C(C)(F)F)n1] |
| | RBr-2; Amine: 3-cyclopropylpyrrolidine-3-carbonitrile |
| | HPLC-F (41-71% MeCN) Yellow solid (13 mg, 14%). LCMS m/z = 453.1 |
| | [M + H]⁺. ¹H NMR (400 MHz, CDCl₃) δ: 9.52 (s, 1H), 8.76 (d, 1H), 8.73 (s, |
| | 1H), 8.07 (s, 1H), 7.79 (d, 1H), 4.20-4.17 (m, 1H), 4.07-4.02 (m, 2H), 3.81- |
| | 3.78 (m, 1H), 2.62-2.59 (m, 1H), 2.36-2.25 (m, 7H), 1.16-1.14 (m, 1H), |
| | 0.75-0.68 (m, 4H). |
| 247 | N-(1-(4-(1,1-difluoroethyl)pyrimidin-2-yl)-3-(3-methoxy-3- |
| | methylpyrrolidin-1-yl)-1H-pyrazolo[4,3-c]pyridin-6-yl)acetamide |
| | 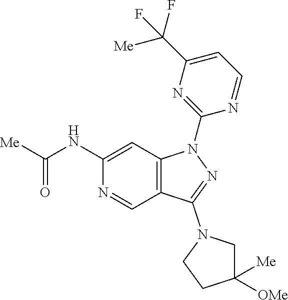 [SMILES: COC1(C)CCN(c2nn(-c3nccc(C(C)(F)F)n3)c3cc(NC(C)=O)ncc23)C1] |
| | RBr-1; Amine: 3-methoxy-3-methylpyrrolidine hydrochloride |
| | HPLC-C (28-58% MeCN). Yellow solid (39.0 mg, 15%). LCMS m/z = |
| | 432.1 [M + H]⁺. ¹H NMR (500 MHz, CDCl₃) δ: 9.37 (s, 1H), 8.95 (d, 1H), |
| | 8.75 (s, 1H), 8.11 (s, 1H), 7.35 (d, 1H), 4.02-3.96 (m, 2H), 3.91-3.89 (m, |
| | 1H), 3.65-3.62 (m, 1H), 3.30 (s, 3H), 2.33-2.24 (m, 7H), 2.00-1.97 (m, 1H), |
| | 1.47 (s, 3H). |

### Example 248: N-(1-(4-(1,1-difluoroethyl)pyrimidin-2-yl)-3-(3-(dimethylamino) pyrrolidin-1-yl)-1H-pyrrolo[3,2-c]pyridin-6-yl)acetamide

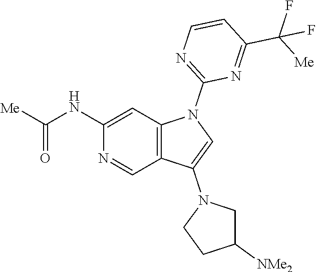 [SMILES: CC(=O)Nc1cc2c(cn1)c(N1CCC(N(C)C)C1)cn2-c1nccc(C(C)(F)F)n1]

To a solution of N-(3-bromo-1-(4-(1,1-difluoroethyl)pyrimidin-2-yl)-1H-pyrrolo[3,2-c]pyridin-6-yl)acetamide (Preparation 386, 200 mg, 0.505 mmol) and N,N-dimethylpyrrolidin-3-amine (173 mg, 1.51 mmol) in DMSO (5 mL) was added K₂CO₃ (209.3 mg, 1.51 mmol), CuI (19.2 mg, 0.101 mmol) and L-proline (23.3 mg, 0.202 mmol) at 25° C. and the mixture stirred at 100° C. for 16 h under N₂. The reaction was treated with H₂O (15 mL) and extracted with DCM/MeOH (v/v=10/1, 15.0 mL×3). The combined organics were washed by brine (20 mL), dried (Na₂SO₄) and evaporated to dryness in vacuo. The residue was purified by column chromatography (DCM/MeOH from 0/1 to 10/1) on silica gel and then purified by HPLC-F (36-66%0 MeCN) to give N-(1-(4-(1,1-difluoroethyl)pyrimidin-2-yl)-3-(3-(dimethylamino)pyrrolidin-1l-yl)-1H-pyrrolo[3,2-c]pyridin-6-yl)acetamide as a yellow solid (44 mg, 20%). LCMS m/z=430.1 [M+H]⁺. ¹H NMR (500 MHz, CDCl₃) δ: 9.47 (s, 1H), 8.81 (d, 1H), 8.68 (s, 1H), 8.49 (s, 1H), 7.31-7.26 (m, 2H), 3.60-3.51 (m, 3H), 3.36-3.34 (m, 1H), 3.00-2.97 (m, 1H), 2.34 (s, 6H), 2.27-2.23 (m, 7H), 2.03-2.00 (m, 1H).

### Example 249: N-(1-(2-(1,1-difluoroethyl)pyrimidin-4-yl)-3-(3-(dimethylamino) pyrrolidin-1-yl)-1H-pyrrolo[3,2-c]pyridin-6-yl)acetamide

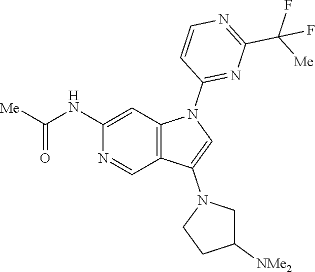 [SMILES: CC(=O)Nc1cc2c(cn1)c(N1CCC(N(C)C)C1)cn2-c1ccnc(C(C)(F)F)n1]

N-(1-(2-(1,1-Difluoroethyl)pyrimidin-4-yl)-3-(3-(dimethylamino)pyrrolidin-1-yl)-1H-pyrrolo[3,2-c]pyridin-6-yl)acetamide was prepared as a yellow solid (25.4 mg, 12%) from N-(3-bromo-1-(2-(1,1-difluoroethyl)pyrimidin-4-yl)-1H-pyrrolo[3,2-c]pyridin-6-yl)acetamide (Preparation 387) and N,N-dimethylpyrrolidin-3-amine using an analogous method to that described for Example 248. LCMS m/z=430.1 [M+H]⁺. ¹H NMR (500 MHz, CDCl₃) δ: 9.36 (s, 1H), 8.76 (d, 1H), 8.70 (s, 1H), 8.32 (s, 1H), 7.29 (d, 1H), 6.70 (s, 1H), 3.61-3.52 (m, 3H), 3.37-3.35 (m, 1H), 2.99-2.97 (m, 1H), 2.35 (s, 6H), 2.28-2.23 (m, 7H), 2.04-2.01 (m, 1H).

### Example 250 and 251: N-(3-((1S,2S)-2-cyanocyclopropyl)-1-(2-(1,1-difluoroethyl)pyrimidin-4-yl)-1H-pyrrolo[3,2-c]pyridin-6-yl)acetamide and N-(3-((1R,2R)-2-cyanocyclopropyl)-1-(2-(1,1-difluoroethyl)pyrimidin-4-yl)-1H-pyrrolo[3,2-c]pyridin-6-yl)acetamide

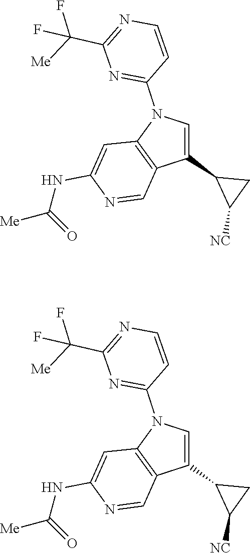 [SMILES: CC(=O)Nc1cc2c(cn1)c([C@H]1C[C@@H]1C#N)cn2-c1ccnc(C(C)(F)F)n1 ; CC(=O)Nc1cc2c(cn1)c([C@@H]1C[C@H]1C#N)cn2-c1ccnc(C(C)(F)F)n1]

Part 1. To a solution of N-(3-bromo-1-(2-(1,1-difluoroethyl)pyrimidin-4-yl)-1H-pyrrolo[3,2-c]pyridin-6-yl)acetamide (Preparation 387, 170 mg, 0.429 mmol) in dioxane (5 mL) and H₂O (0.5 mL) was added 2-(4,4,5,5-tetramethyl-1,3,2-dioxaborolan-2-yl)cyclopropane-1-carbonitrile (Preparation 375, 414 mg, 2.15 mmol), K₂CO₃ (178 mg, 1.29 mmol) and Pd(dtbpf)Cl₂ (27.9 mg, 0.043 mol) and the mixture stirred at 80° C. for 1 h under N₂. The mixture was concentrated and purified by HPLC-F (28-58% MeCN) to give N-(3-(2-cyanocyclopropyl)-1-(2-(1,1-difluoroethyl)pyrimidin-4-yl)-1H-pyrrolo[3,2-c]pyridin-6-yl)acetamide as a white solid (40 mg, 24%).

Part 2. N-(3-(2-cyanocyclopropyl)-1-(2-(1,1-difluoroethyl)pyrimidin-4-yl)-1H-pyrrolo[3,2-c]pyridin-6-yl)acetamide (Part 1, 40 mg) was purified using chiral-SFC chromatography (Diacel Chiralpak AD, 250×30 mm, 10 m; 45% EtOH (+0.1% NH₄OH) in CO₂) to afford the title compounds.

Peak 1. White solid (18.4 mg, 46%); LCMS m/z=383.0 [M+H]⁺. ¹H NMR (500 MHz, CDCl₃) δ: 9.18 (s, 1H), 8.89 (d, 1H), 8.67 (s, 1H), 8.11 (s, 1H), 7.62 (s, 1H), 7.52 (d, 1H), 2.75-2.70 (m, 1H), 2.27 (s, 3H), 2.24-2.16 (m, 3H), 1.76-1.73 (m, 1H), 1.68-1.64 (m, 2H).

Peak 2. White solid (15.7 mg, 39%); LCMS m/z=383.0 [M+H]⁺. ¹H NMR (500 MHz, CDCl₃) δ: 9.18 (s, 1H), 8.89 (d, 1H), 8.67 (s, 1H), 8.13 (s, 1H), 7.62 (s, 1H), 7.52 (d, 1H), 2.75-2.70 (m, 1H), 2.27 (s, 3H), 2.24-2.16 (m, 3H), 1.76-1.73 (m, 1H), 1.68-1.64 (m, 2H).

### Example 252: N-(1-(5-(tetrahydrofuran-3-yl)thiazol-2-yl)-1H-pyrazolo[4,3-c]pyridin-6-yl)acetamide

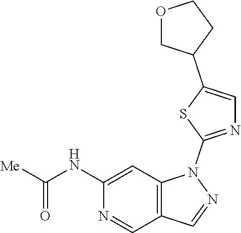 [SMILES: CC(=O)Nc1cc2c(cn1)cnn2-c1ncc(C2CCOC2)s1]

NaH (10.5 mg, 0.439 mmol, 60%) was added to a solution of 2-chloro-5-(tetrahydrofuran-3-yl)thiazole (100 mg, 0.527 mmol) and N-(1H-pyrazolo[4,3-c]pyridin-6-yl)acetamide (Preparation 2, 77.4 mg, 0.439 mmol) in DMF (5 mL) at 0° C. and the resulting mixture was stirred at 130° C. for 8 h under N₂. The mixture was quenched with H₂O (0.2 mL) and purified by HPLC-C (30-60% MeCN) to give N-(1-(5-(tetrahydrofuran-3-yl)thiazol-2-yl)-1H-pyrazolo[4,3-c]pyridin-6-yl)acetamide as a white solid (7.7 mg, 5%). LCMS m/z=330.1 [M+H]⁺. ¹H NMR (500 MHz, CDCl₃) δ: 8.97 (s, 1H), 8.94 (s, 1H), 8.41 (s, 1H), 7.97 (s, 1H), 7.43 (s, 1H), 4.15-4.08 (m, 2H), 3.97-3.95 (m, 1H), 3.81-3.77 (m, 2H), 2.51-2.46 (m, 1H), 2.24 (s, 3H), 2.09-2.04 (m, 1H).

### Example 253: N-(1-(4-(tetrahydrofuran-3-yl)thiazol-2-yl)-1H-pyrazolo[4,3-c]pyridin-6-yl)acetamide

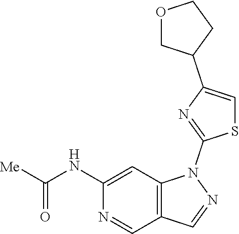 [SMILES: CC(=O)Nc1cc2c(cn1)cnn2-c1nc(C2CCOC2)cs1]

To a solution of 2-bromo-4-(tetrahydrofuran-3-yl)thiazole (80 mg, 0.342 mmol), N-(1H-pyrazolo[4,3-c]pyridin-6-yl)acetamide (Preparation 2, 60.2 mg, 0.342 mol) in dioxane (3 mL) was added N¹,N²-dimethylethane-1,2-diamine (6.0 mg, 0.068 mmol), CuI (26 mg, 0.137 mmol) and K₂CO₃ (94.5 mg, 0.683 mmol) and the reaction mixture stirred at 90° C. for 2 h under N₂. The mixture was concentrated under reduced pressure and the residue purified by prep-HPLC-G (25-55% MeCN) to give N-(1-(4-(tetrahydrofuran-3-yl)thiazol-2-yl)-1H-pyrazolo[4,3-c]pyridin-6-yl)acetamide as a white solid (25.1 mg, 22%). LCMS m/z=330.1 [M+H]⁺. ¹H NMR (400 MHz, CDCl₃) δ: 9.30 (s, 1H), 8.78 (s, 1H), 8.19-8.18 (m, 2H), 6.75 (s, 1H), 4.25-4.15 (m, 2H), 4.03-3.93 (m, 2H), 3.64-3.63 (m, 1H), 2.43-2.33 (m, 2H), 2.27 (s, 3H).

Example 254-257: The title compounds were prepared from the appropriate acetamide and aryl halide using an analogous method to that described for Example 253.

| Example No | Name/Structure/Acetamide/thiazole/Data |
|---|---|
| 254 | N-(1-(5-methyl-4-(tetrahydrofuran-3-yl)thiazol-2-yl)-1H-pyrazolo[4,3- |
| | c]pyridin-6-yl)acetamide |
| | 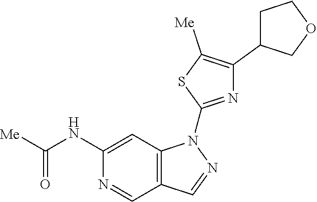 [SMILES: CC(=O)Nc1cc2c(cn1)cnn2-c1nc(C2CCOC2)c(C)s1] |
| | Reactants: N-(1H-pyrazolo[4,3-c]pyridin-6-yl)acetamide (Preparation 2) |
| | and 2-bromo-5-methyl-4-(tetrahydrofuran-3-yl)thiazole |
| | HPLC-C (30-60% MeCN). White solid (8.5 mg, 8%). LCMS m/z = 344.2 |
| | [M + H]⁺. ¹H NMR (400 MHz, CDCl₃) δ: 9.25 (s, 1H), 8.76 (s, 1H), 8.16- |
| | 8.14 (m, 2H), 4.32-4.28 (m, 1H), 4.20-4.16 (m, 1H), 4.09-4.06 (m, 1H), |
| | 3.90-3.85 (m, 1H), 3.86-3.54 (m, 1H), 2.50-2.46 (m, 1H), 2.45 (s, 3H), |
| | 2.41-2.33 (m, 1H), 2.26 (s, 3H). |
| 255 | N-(3-methyl-1-(4-(tetrahydrofuran-3-yl)thiazol-2-yl)-1H-pyrazolo[4,3- |
| | c]pyridin-6-yl)acetamide |
| | 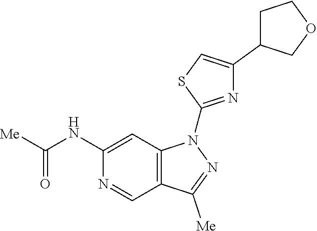 [SMILES: CC(=O)Nc1cc2c(cn1)c(C)nn2-c1nc(C2CCOC2)cs1] |
| | Reactants: N-(3-methyl-1H-pyrazolo[4,3-c]pyridin-6-yl)acetamide |
| | (Preparation 6) and 2-bromo-4-(tetrahydrofuran-3-yl)thiazole |
| | HPLC-C (27-57% MeCN). White solid (3.6 mg, 8%). LCMS m/z = 344.2 |
| | [M + H]⁺. ¹H NMR (500 MHz, MeOH-d₄) δ: 9.18 (s, 1H), 8.83 (s, 1H), 6.99 |
| | (s, 1H), 4.22-4.16 (m, 2H), 4.02-4.00 (m, 1H), 3.95-3.91 (m, 1H), 3.65-3.63 |
| | (m, 1H), 2.65 (s, 3H), 2.42-2.36 (m, 2H), 2.26 (s, 3H). |
| 256 | N-(3-methyl-1-(5-methyl-4-(tetrahydrofuran-3-yl)thiazol-2-yl)-1H- |
| | pyrazolo[4,3-c]pyridin-6-yl)acetamide |
| | 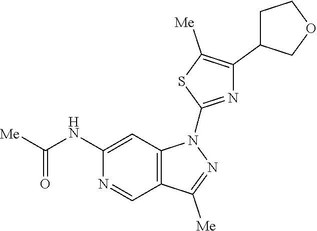 [SMILES: CC(=O)Nc1cc2c(cn1)c(C)nn2-c1nc(C2CCOC2)c(C)s1] |
| | Reactants: N-(3-methyl-1H-pyrazolo[4,3-c]pyridin-6-yl)acetamide |
| | (Preparation 6) and 2-bromo-5-methyl-4-(tetrahydrofuran-3-yl)thiazole |
| | HPLC-C (34-64% MeCN). White solid (8 mg, 14%). LCMS m/z = 358.1 |
| | [M + H]⁺. ¹H NMR (500 MHz, CDCl₃) δ: 9.18 (s, 1H), 8.65 (s, 1H), 8.15 (s, |
| | 1H), 4.30-4.27 (m, 1H), 4.19-4.15 (m, 1H), 4.08-4.06 (m, 1H), 3.89-3.85 |
| | (m, 1H), 3.54-3.52 (m, 1H), 2.63 (s, 3H), 2.48-2.44 (m, 1H), 2.39 (s, 3H), |
| | 2.33-2.31 (m, 1H), 2.25 (s, 3H). |
| 257 | N-(1-(4-(1, 1-difluoroethyl)-5-methylthiazol-2-yl)-1H-pyrazolo[4,3- |
| | c]pyridin-6-yl)acetamide |
| | 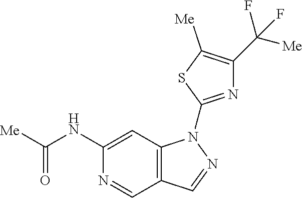 [SMILES: CC(=O)Nc1cc2c(cn1)cnn2-c1nc(C(C)(F)F)c(C)s1] |
| | Reactants: N-(3-methyl-1H-pyrazolo[4,3-c]pyridin-6-yl)acetamide |
| | (Preparation 6) and 2-bromo-4-(1,1-difluoroethyl)-5-methylthiazole |
| | HPLC-C (39-69% MeCN). White solid (4.2 mg, 3%). LCMS m/z = 338.1 |
| | [M + H]⁺. ¹H NMR (400 MHz, CDCl₃) δ: 9.36 (s, 1H), 8.81-8.78 (m, 2H), |
| | 8.24 (s, 1H), 2.62 (t, 3H), 2.30 (s, 3H), 2.25 (t, 3H). |

### Example 258: N-(1-(5-(3-methyltetrahydrofuran-3-yl)thiazol-2-yl)-1H-pyrazolo[4,3-c]pyridin-6-yl)acetamide

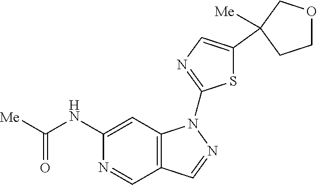 [SMILES: CC(=O)Nc1cc2c(cn1)cnn2-c1ncc(C2(C)CCOC2)s1]

A mixture of 2-chloro-5-(3-methyltetrahydrofuran-3-yl)thiazole (80 mg, 0.322 mmol), N-(1H-pyrazolo[4,3-c]pyridin-6-yl)acetamide (Preparation 2, 56.8 mg, 0.322 mmol), Brettphos Pd G₃ (58.5 mg, 0.064 mmol) and Cs₂CO₃ (210.1 mg, 0.645 mmol) in toluene (5 mL) was stirred at 90° C. under N₂ for 2 h. The mixture was concentrated under reduced pressure and the residue was purified by prep-HPLC-C (29-59% MeCN) to give N-(1-(5-(3-methyltetrahydrofuran-3-yl)thiazol-2-yl)-1H-pyrazolo[4,3-c]pyridin-6-yl)acetamide as a white solid (7.9 mg, 7%). LCMS m/z=344.2 [M+H]⁺. ¹H NMR (500 MHz, MeOH-d₄) δ: 9.13 (s, 1H), 8.86 (s, 1H), 8.34 (s, 1H), 7.49 (s, 1H), 4.07-4.05 (m, 2H), 3.92 (d, 1H), 3.74 (d, 1H), 2.34-2.28 (m, 1H), 2.23 (s, 3H), 2.20-2.17 (m, 1H), 1.59 (s, 3H).

### Example 259: N-(1-(4-(2-hydroxypropan-2-yl)-5-methylthiazol-2-yl)-1H-pyrazolo[4,3-c]pyridin-6-yl)acetamide

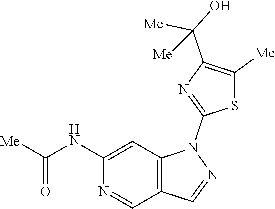 [SMILES: CC(=O)Nc1cc2c(cn1)cnn2-c1nc(C(C)(C)O)c(C)s1]

Part A: To a solution of ethyl 2-chloro-5-methylthiazole-4-carboxylate (500 mg, 2.4 mmol) in dioxane (5 mL) was added N-(1H-pyrazolo[4,3-c]pyridin-6-yl)acetamide (Preparation 2, 428 mg, 2.43 mmol), Xantphos (281.4 mg, 0.486 mmol), Cs₂CO₃ (1.6 g, 4.86 mmol) and Pd₂(dba)₃ (222.6 mg, 0.243 mmol) and the resulting mixture was stirred at 110° C. for 16 h under N₂. The solids were removed by filtration and the filtrate was concentrated under reduced pressure and the residue purified by chromatography on silica gel (PE/EtOAc=3/1) to give ethyl 2-(6-acetamido-1H-pyrazolo[4,3-c]pyridin-1-yl)-5-methylthiazole-4-carboxylate as a yellow solid (350 mg, 42%). ¹H NMR (400 MHz, CDCl₃) δ: 9.33 (s, 1H), 8.78 (s, 1H), 8.49 (br s, 1H), 8.23 (s, 1H), 4.36 (t, 2H), 2.76 (s, 3H), 2.29 (s, 3H), 1.38 (t, 3H).

Part B: To a solution of ethyl 2-(6-acetamido-1H-pyrazolo[4,3-c]pyridin-1-yl)-5-methylthiazole-4-carboxylate (200 mg, 0.579 mmol) in THF (3 mL) was added MeMgBr (3 M, 0.386 mL) at 0° C. under N₂ and the resulting mixture stirred at 0° C. for 16 h under N₂. The mixture was quenched by aq. NH₄Cl (1 mL), concentrated and purified by HPLC-C (23-53% MeCN) to give N-(1-(4-(2-hydroxypropan-2-yl)-5-methylthiazol-2-yl)-1H-pyrazolo[4,3-c]pyridin-6-yl)acetamide as a yellow oil (8 mg, 4.2%). LCMS m/z=332.1 [M+H]⁺. ¹H NMR (400 MHz, CDCl₃) δ: 9.22 (s, 1H), 8.75 (s, 1H), 8.30 (br s, 1H), 8.15 (s, 1H), 2.56 (s, 3H), 2.27 (s, 3H), 1.71 (s, 6H).

### Example 260: N-(1-(5-methyl-4-(tetrahydrofuran-3-yl)thiazol-2-yl)-1H-pyrrolo[3,2-c]pyridin-6-yl)acetamide

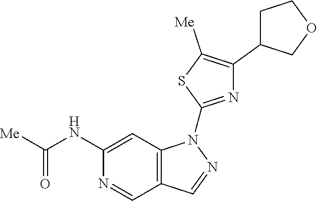 [SMILES: CC(=O)Nc1cc2c(cn1)cnn2-c1nc(C2CCOC2)c(C)s1]

Acetyl chloride (5.2 mg, 0.067 mmol) was added dropwise to the solution of 1-(5-methyl-4-(tetrahydrofuran-3-yl)thiazol-2-yl)-1H-pyrrolo[3,2-c]pyridin-6-amine (Preparation 379, 10.0 mg, 0.033 mmol) and DIPEA (5.2 mg, 0.067 mmol) in DMF (3 mL) at 0° C. under N₂ and stirred at 25° C. for 1 h. The mixture was concentrated in vacuo and the residue was purified by prep-HPLC-G (28-58% MeCN) to give N-(1-(5-methyl-4-(tetrahydrofuran-3-yl)thiazol-2-yl)-1H-pyrrolo[3,2-c]pyridin-6-yl)acetamide as a white solid (6.0 mg, 53%). LCMS m/z=343.1 [M+H]⁺. ¹H NMR (400 MHz, CDCl₃) δ: 9.02 (s, 1H), 8.56 (s, 1H) 8.28 (s, 1H) 7.67 (d, 1H), 6.69 (d, 1H), 4.18-4.12 (m, 2H), 4.02-3.99 (m, 1H), 3.89-3.84 (m, 1H), 3.54-3.50 (m, 1H), 2.43 (s, 3H), 2.38-2.27 (m, 2H), 2.24 (s, 3H).

### Example 261: N-(1-(3-(tetrahydrofuran-3-yl)-1,2,4-thiadiazol-5-yl)-1H-pyrazolo[4,3-c]pyridin-6-yl)acetamide

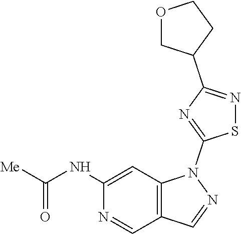 [SMILES: CC(=O)Nc1cc2c(cn1)cnn2-c1nc(C2CCOC2)ns1]

To a solution of N-(1H-pyrazolo[4,3-c]pyridin-6-yl)acetamide (Preparation 2, 73.9 mg, 0.420 mmol) and 5-chloro-3-(tetrahydrofuran-3-yl)-1,2,4-thiadiazole (80 mg, 0.420 mmol) in toluene (3 mL) was added BrettPhos Pd G₃ (38.1 mg, 0.042 mmol) and Cs₂CO₃ (273.4 mg, 0.839 mmol) and the resulting mixture stirred at 90° C. for 12 h under N₂. The mixture was concentrated and purified by HPLC-C (26-56% MeCN) to give N-(1-(3-(tetrahydrofuran-3-yl)-1,2,4-thiadiazol-5-yl)-1H-pyrazolo[4,3-c]pyridin-6-yl)acetamide as a white solid (9.1 mg, 6.6%). LCMS m/z=331.2 [M+H]⁺. ¹H NMR (500 MHz, CDCl₃) δ: 9.28 (s, 1H), 8.79 (d, 1H), 8.28 (s, 1H), 8.17 (s, 1H), 4.29-4.25 (m, 1H), 4.17-4.15 (m, 2H), 4.14-4.03 (m, 1H), 3.84-3.83 (m, 1H), 2.57-2.46 (m, 2H), 2.29 (s, 3H).

### Example 262: N-(1-(5-(7-oxabicyclo[2.2.1]heptan-2-yl)-1,3,4-thiadiazol-2-yl)-1H-pyrazolo[4,3-c]pyridin-6-yl)acetamide

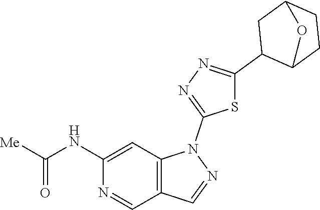 [SMILES: CC(=O)Nc1cc2c(cn1)cnn2-c1nnc(C2CC3CCC2O3)s1]

N-(1-(5-(7-oxabicyclo[2.2.1]heptan-2-yl)-1,3,4-thiadiazol-2-yl)-1H-pyrazolo[4,3-c]pyridin-6-yl)acetamide was prepared as a white solid (30.6 mg, 28%) from 2-(7-oxabicyclo[2.2.1]heptan-2-yl)-5-chloro-1,3,4-thiadiazole and N-(1H-pyrazolo[4,3-c]pyridin-6-yl)acetamide (Preparation 2) using an analogous method to that described for Example 261. LCMS m/z=357.2 [M+H]⁺. ¹H NMR (500 MHz, CDCl₃) δ: 9.19 (s, 1H), 8.79 (s, 1H), 8.23-8.22 (m, 2H), 4.80 (t, 1H), 4.63 (d, 1H), 3.65-3.61 (m, 1H), 2.27 (s, 3H), 2.22-2.18 (m, 1H), 1.87-1.83 (m, 4H), 1.63-1.59 (m, 1H).

### Example 263: N-(1-(4-(tetrahydrofuran-3-yl)thiazol-2-yl)-1H-pyrrolo[3,2-c]pyridin-6-yl)acetamide

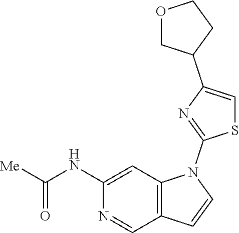 [SMILES: CC(=O)Nc1cc2c(ccn2-c2nc(C3CCOC3)cs2)cn1]

A solution of 2-(6-chloro-1H-pyrrolo[3,2-c]pyridin-1-yl)-4-(tetrahydrofuran-3-yl)thiazole (Preparation 380, 50.0 mg, 0.164 mmol), acetamide (193.2 mg, 3.27 mmol) t-BuBrettPhos Pd G3 (14.0 mg, 16.35 umol) and Cs₂CO₃ (266.4 mg, 0.818 mmol) in t-BuOH (10 mL) was stirred at 110° C. for 2 h under microwave irradiation under N₂. The cooled mixture was concentrated in vacuo and purified by Prep-HPLC-G (Gradient: 30 to 60% MeCN) to give N-(1-(4-(tetrahydrofuran-3-yl)thiazol-2-yl)-1H-pyrrolo[3,2-c]pyridin-6-yl)acetamide (6 mg, 11.2% yield) as a white solid. LCMS m/z=329.0 [M+H]^{+ 1}H NMR (400 MHz, CDCl₃) δ: ppm 9.09 (s, 1H), 8.58 (s, 1H), 8.24 (s, 1H), 7.71 (d, J=3.6 Hz, 1H), 6.79 (s, 1H), 6.72 (d, J=3.2 Hz, 1H), 4.20-4.17 (m, 1H), 4.11-4.09 (m, 1H), 3.99-3.91 (m, 2H), 3.64-3.61 (m, 1H), 2.41-2.37 (m, 2H), 2.45 (s, 3H).

### Example 264: N-(1-(5-(3-methoxytetrahydrofuran-3-yl)-1,2,4-oxadiazol-3-yl)-1H-pyrazolo[4,3-c]pyridin-6-yl)acetamide

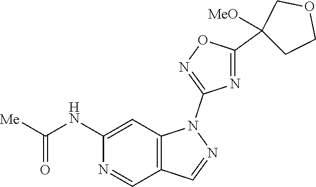 [SMILES: COC1(c2nc(-n3ncc4cnc(NC(C)=O)cc43)no2)CCOC1]

To a solution of N-(1H-pyrazolo[4,3-c]pyridin-6-yl)acetamide (Preparation 2, 56.6 mg, 0.321 mmol) in THF (5 mL) was added 3-bromo-5-(3-methoxytetrahydrofuran-3-yl)-1,2,4-oxadiazole (80 mg, 0.321 mmol) and t-BuOK (54.1 mg, 0.482 mmol) and the resulting mixture was stirred at 50° C. for 12 h. The mixture was concentrated under reduced pressure and the residue purified by prep-HPLC-G (8-38% MeCN) to afford N-(1-(5-(3-methoxytetrahydrofuran-3-yl)-1,2,4-oxadiazol-3-yl)-1H-pyrazolo[4,3-c]pyridin-6-yl)acetamide as a white solid (3.2 mg, 2.9%). LCMS m/z=345.2 [M+H]⁺. ¹H NMR (500 MHz, CDCl₃) δ: 9.08 (s, 1H), 8.82 (s, 1H), 8.35 (s, 1H), 8.20 (br s, 1H), 4.31-4.29 (m, 1H), 4.24-4.22 (m, 1H), 4.15-4.13 (m, 2H), 3.40 (s, 3H), 2.76-2.70 (m, 1H), 2.63-2.60 (m, 1H), 2.28 (s, 3H).

### Example 265: N-(1-(5-(3-methyltetrahydrofuran-3-yl)-1,2,4-oxadiazol-3-yl)-1H-pyrazolo[4,3-c]pyridin-6-yl)acetamide

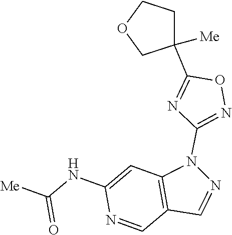 [SMILES: CC(=O)Nc1cc2c(cn1)cnn2-c1noc(C2(C)CCOC2)n1]

N-(1-(5-(3-Methyltetrahydrofuran-3-yl)-1,2,4-oxadiazol-3-yl)-1H-pyrazolo[4,3-c]pyridin-6-yl)acetamide was prepared as a white solid (6.1 mg, 4.3%) from N-(1H-pyrazolo[4,3-c]pyridin-6-yl)acetamide (Preparation 2) and 3-bromo-5-(3-methyltetrahydrofuran-3-yl)-1,2,4-oxadiazole using an analogous method as described for Example 264. LCMS m/z=329.2 [M+H]⁺. ¹H NMR (400 MHz, CDCl₃) δ: 9.08 (s, 1H), 8.81 (d, 1H), 8.33 (s, 1H), 8.15 (br s, 1H), 4.32-4.29 (m, 1H), 4.14-4.08 (m, 2H), 3.86-3.82 (m, 1H), 2.83-2.76 (m, 1H), 2.28 (s, 3H), 2.18-2.11 (m, 1H), 1.69 (s, 3H).

### Example 266: N-(1-(3-(1-amino-2,2,2-trifluoroethyl)phenyl)-1H-pyrazolo[4,3-c]pyridin-6-yl)acetamide

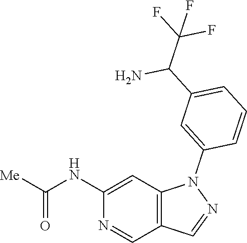 [SMILES: CC(=O)Nc1cc2c(cn1)cnn2-c1cccc(C(N)C(F)(F)F)c1]

To a solution of tert-butyl (1-(3-(6-acetamido-1H-pyrazolo[4,3-c]pyridin-1-yl)phenyl)-2,2,2-trifluoroethyl)carbamate (Preparation 383, 20 mg, 0.045 mmol) in DCM (2.5 mL) was added TFA (0.5 mL) and the resulting mixture was stirred at 30° C. for 1 h. The reaction mixture was concentrated under reduced pressure and the residue was purified by prep-HPLC-G (22-52% MeCN) to give N-(1-(3-(1-amino-2,2,2-trifluoroethyl)phenyl)-1H-pyrazolo[4,3-c]pyridin-6-yl)acetamide as a white solid (6 mg, 39%). LCMS m/z=350.2 [M+H]⁺. ¹H NMR (400 MHz, CDCl₃) δ: 8.81 (s, 1H), 8.61 (s, 1H), 8.25 (s, 1H), 8.19 (br s, 1H), 7.87 (s, 1H), 7.77-7.74 (m, 1H), 7.58 (t, 1H), 7.47 (d, 1H), 4.55-4.53 (m, 1H), 2.24 (s, 3H), 1.89 (br s, 2H).

### Example 267: N-(1-(3-(3-(hydroxymethyl)oxetan-3-yl)phenyl)-1H-pyrazolo[4,3-c]pyridin-6-yl)acetamide

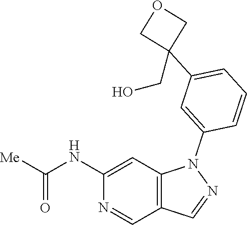 [SMILES: CC(=O)Nc1cc2c(cn1)cnn2-c1cccc(C2(CO)COC2)c1]

To a solution of N-(1-(3-(3-(((tert-butyldimethylsilyl)oxy)methyl)oxetan-3-yl)phenyl)-1H-pyrazolo[4,3-c]pyridin-6-yl)acetamide (Preparation 385, 68.1 mg, 0.150 mmol) in DCM (3 mL) was added TFA (1.5 g, 13.06 mmol) and the resulting mixture was stirred at 25° C. for 12 h. The mixture was purified by HPLC-C (26-56% MeCN) to give N-(1-(3-(3-(hydroxymethyl)oxetan-3-yl)phenyl)-1H-pyrazolo[4,3-c]pyridin-6-yl)acetamide as a white solid (38.7 mg, 76%). LCMS m/z=339.2 [M+H]⁺. ¹H NMR (400 MHz, CDCl₃) δ: 8.77 (s, 1H), 8.56 (s, 1H), 8.29 (s, 1H), 8.23 (s, 1H), 7.64 (d, 1H), 7.57 (d, J=8.0 Hz, 1H), 7.49 (s, 1H), 7.15 (d, 1H), 5.03 (d, 2H), 4.82 (d, 2H), 4.18 (d, 2H), 2.50 (br s, 1H), 2.23 (s, 3H).

### Example 268: N-(3-([1,3′-bipyrrolidin]-1′-yl)-1-(2-(1,1-difluoroethyl)-6-methylpyrimidin-4-yl)-1H-pyrazolo[4,3-c]pyridin-6-yl)acetamide

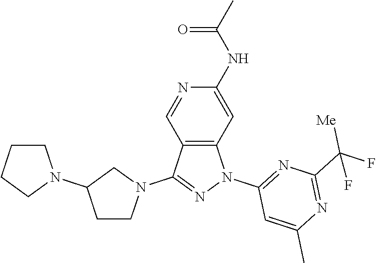 [SMILES: CC(=O)Nc1cc2c(cn1)c(N1CCC(N3CCCC3)C1)nn2-c1cc(C)nc(C(C)(F)F)n1]

A suspension of 1-(pyrrolidin-3-yl)pyrrolidine (137.7 mg, 0.98 mmol), N-(1-(2-(1,1-difluoroethyl)-6-methylpyrimidin-4-yl)-3-iodo-1H-pyrazolo[4,3-c]pyridin-6-yl)acetamide (Preparation 392, 150 mg, 0.33 mmol), 2-(2,6-difluoroanilino)-2-oxo-acetic acid (13.17 mg, 0.065 mmol) and K₃PO₄ (208.5 mg, 0.98 mmol) in dry DMSO (2 mL) was sparged with N₂ for 5 min. CuI (12.47 mg, 0.065 mmol) was added and the reaction stirred at 100° C. for 2 h. The cooled mixture was diluted with water and extracted with EtOAc (3×). The combined organic layers were washed with brine, dried (MgSO₄), filtered and concentrated in vacuo. The residue was purified by silica gel chromatography (0-10% MeOH in DCM) to give N-(3-([1,3′-bipyrrolidin]-1′-yl)-1-(2-(1,1-difluoroethyl)-6-methylpyrimidin-4-yl)-1H-pyrazolo[4,3-c]pyridin-6-yl)acetamide (63 mg, 38.9% yield) as a pale orange solid. LCMS m/z=471.2 [M+H]⁺1H NMR (500 MHz, CDCl₃) δ ppm 9.44 (br s, 1H), 8.68 (br s, 1H), 8.24 (br s, 1H), 7.61 (s, 1H), 3.90-4.01 (m, 2H), 3.66-3.80 (m, 2H), 3.23 (br s, 1H), 2.89 (br s, 4H), 2.66 (s, 4H), 2.31-2.37 (m, 2H), 2.26-2.31 (m, 6H), 1.98 (br s, 4H).

Examples 269 to 273: The compounds in the following table were prepared from N-(3-bromo-1-(2-(1,1-difluoroethyl)-6-ethylpyrimidin-4-yl)-1H-pyrazolo[4,3-c]pyridin-6-yl)acetamide (Preparation 393) and amine, following the procedure described in Example 268.

| Example | Name, Structure, Starting materials (SM), | |
|---|---|---|
| Number | yield | Data |
| 269 | (S)-N-(3-([1,3′-bipyrrolidin]-1′-yl)-1-(2-(1,1- difluoroethyl)-6-ethylpyrimidin-4-yl)-1H- pyrazolo[4,3-c]pyridin-6-yl)acetamide 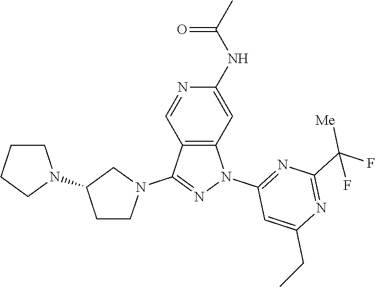 [SMILES: CCc1cc(-n2nc(N3CC[C@H](N4CCCC4)C3)c3cnc(NC(C)=O)cc32)nc(C(C)(F)F)n1] | LCMS m/z = 485.2 [M + H]⁺. ¹H NMR (500 MHz, CDCl₃) δ ppm 9.42 (br s, 1 H), 8.67 (s, 1 H), 8.56 (br s, 1 H), 7.60 (s, 1 H), 3.85-3.95 (m, 2 H), 3.70 (td, J = 9.3, 7.0 Hz, 1 H), 3.53- 3.63 (m, 1 H), 2.86-2.99 (m, 3 H), 2.65 (br d, J = 2.7 Hz, 4 H), 2.21-2.32 (m, 7 H), 2.09 (dq, J = 12.1, 9.0 Hz, 1 H), 1.85 (br s, 4 H), 1.37 (t, J = 7.6 Hz, 3 H) |
| | SM: 1-((3S)-pyrrolidin-3-yl)pyrrolidine | |
| | hydrochloride | |
| | yellow solid, 122 mg, 32.8%, | |
| 270 | (S)-N-(3-([1,3′-bipyrrolidin]-1′-yl)-1-(2-(1,1- difluoroethyl)-6-ethylpyrimidin-4-yl)-1H- pyrazolo[4,3-c]pyridin-6-yl)acetamide 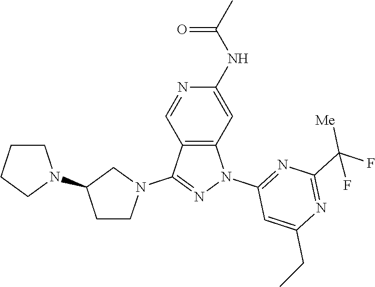 [SMILES: CCc1cc(-n2nc(N3CC[C@@H](N4CCCC4)C3)c3cnc(NC(C)=O)cc32)nc(C(C)(F)F)n1] | LCMS m/z = 485.2 [M + H]⁺. ¹H NMR (500 MHz, CDCl₃) δ ppm 9.49 (br s, 1 H), 8.73 (s, 1 H), 8.11 (s, 1 H), 7.64 (s, 1 H), 3.96 (br t, J-8.1 Hz, 2 H), 3.70-3.83 (m, 1 H), 3.62 (br t, J = 8.5 Hz, 1 H), 2.99 (dt, J = 15.0, 7.3 Hz, 1 H), 2.91 (q, J-7.6 Hz, 2 H), 2.62-2.73 (m, 4 H), 2.24- 2.33 (m, 7 H), 2.09-2.17 (m, 1 H), 1.87 (br s, 4 H), 1.39 (t, J = 7.5 Hz, 3 H). |
| | SM: 1-((3R)-pyrrolidin-3-yl)pyrrolidine | |
| | hydrochloride | |
| | Yellow solid, 473 mg, 43.8% | |
| 271 | (R)-N-(3-(3-(diethylamino)pyrrolidin-1-yl)-1-(2- (1,1-difluoroethyl)-6-ethylpyrimidin-4-yl)-1H- pyrazolo[4,3-c]pyridin-6-yl)acetamide 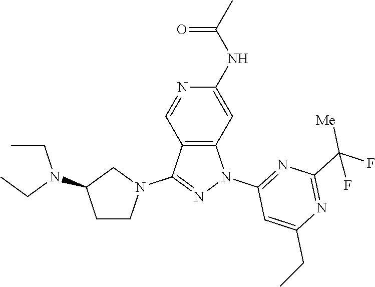 [SMILES: CCc1cc(-n2nc(N3CC[C@@H](N(CC)CC)C3)c3cnc(NC(C)=O)cc32)nc(C(C)(F)F)n1] | LCMS m/z = 487.2 [M + H]⁺. ¹H NMR (400 MHz, CDCl₃) δ ppm 9.48 (br s, 1 H), 8.74 (s, 1 H), 8.11 (s, 1 H), 7.64 (s, 1 H), 3.90-4.00 (m, 2 H), 3.70- 3.77 (m, 1 H), 3.46-3.57 (m, 2 H), 2.91 (q, J = 7.5 Hz, 2 H), 2.67-2.81 (m, 4 H), 2.22-2.34 (m, 7 H), 1.96- 2.14 (m, 1 H), 1.39 (t, J = 7.8 Hz, 3 H), 1.10 (t, J = 7.3 Hz, 6 H) |
| | SM: (3R)-N,N-diethylpyrrolidin-3-amine | |
| | hydrochloride | |
| | Brown solid, 259 mg, 61.4% | |
| 272 | (S)-N-(3-(3-(diethylamino)pyrrolidin-1-yl)-1-(2- (1,1-difluoroethyl)-6-ethylpyrimidin-4-yl)-1H- pyrazolo[4,3-c]pyridin-6-yl)acetamide 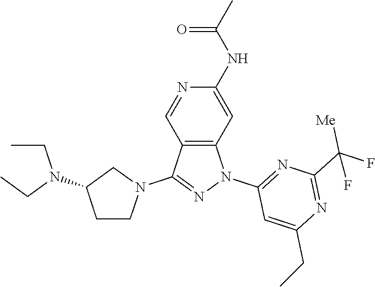 [SMILES: CCc1cc(-n2nc(N3CC[C@H](N(CC)CC)C3)c3cnc(NC(C)=O)cc32)nc(C(C)(F)F)n1] | LCMS m/z = 487.2 [M + H]⁺, ¹H NMR (500 MHz, CDCl₃) δ ppm 9.49 (br s, 1 H), 8.73 (s, 1 H), 8.13 (s, 1 H), 7.64 (s, 1 H), 3.88-4.04 (m, 2 H), 3.66- 3.82 (m, 1 H), 3.46-3.58 (m, 2 H), 2.91 (q, J-7.6 Hz, 2 H), 2.68-2.81 (m, 4 H), 2.24-2.33 (m, 7 H), 1.95- 2.14 (m, 1 H), 1.39 (t, J = 7.6 Hz, 3 H), 1.10 (t, J = 7.0 Hz, 6 H). |
| | SM: (3S)-N,N-diethylpyrrolidin-3-amine | |
| | hydrochloride | |
| | 284 mg, 67.4% as a yellow solid | |
| 273^{A} | N-(1-(2-(1, 1-difluoroethyl)-6-ethylpyrimidin-4- yl)-3-(3-(ethyl(methyl)amino)-3- methylpyrrolidin-1-yl)-1H-pyrazolo[4,3- c]pyridin-6-yl)acetamide 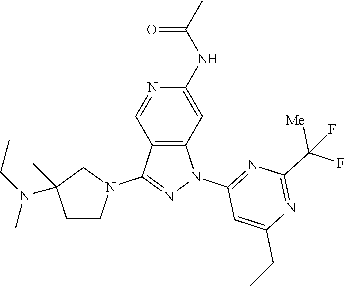 [SMILES: CCc1cc(-n2nc(N3CCC(C)(N(C)CC)C3)c3cnc(NC(C)=O)cc32)nc(C(C)(F)F)n1] | LCMS m/z = 487.3 [M + H]⁺; 1H NMR (400 MHz, MeOD-d₄) δ ppm: 9.36 (s, 1H), 8.89 (s, 1H), 7.71 (s, 1H), 3.96-4.11 (m, 4H), 2.86-2.93 (m, 6H), 2.37-2.69 (m, 3H), 2.23 (s, 3H), 2.22 (t, J = 1 8.8 Hz, 3H), 1.57 (s, 3H), 1.46 (t, J = 7.2 Hz, 3H), 1.36 (t, J = 7.6 Hz, 3H). |
| | SM: N-ethyl-N,3-dimethylpyrrolidin-3-amine | |
| | (Preparation X) | |
| | 28.7 mg, 25.1% yield as yellow solid | |

A-compound purified by Prep-HPLC-C (gradient: 40% to 70% MeCN)

### Examples 274 and 275: (S)—N-(1-(2-(1,1-difluoroethyl)-6-ethylpyrimidin-4-yl)-3-(3-(dimethylamino)-3-methylpyrrolidin-1-yl)-1H-pyrazolo[4,3-c]pyridin-6-yl)acetamide and (R)—N-(1-(2-(1,1-difluoroethyl)-6-ethylpyrimidin-4-yl)-3-(3-(dimethylamino)-3-methylpyrrolidin-1-yl)-1H-pyrazolo[4,3-c]pyridin-6-yl)acetamide

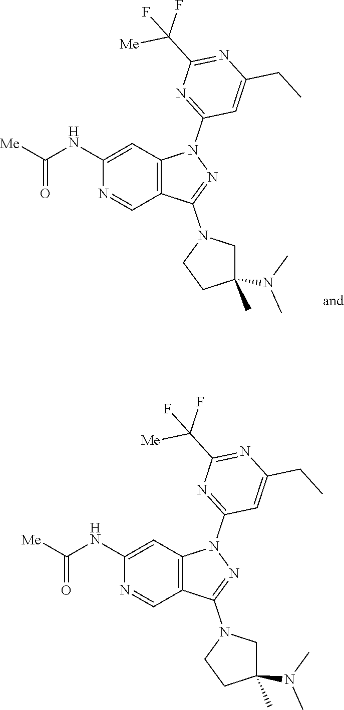 [SMILES: CCc1cc(-n2nc(N3CC[C@@](C)(N(C)C)C3)c3cnc(NC(C)=O)cc32)nc(C(C)(F)F)n1 ; CCc1cc(-n2nc(N3CC[C@](C)(N(C)C)C3)c3cnc(NC(C)=O)cc32)nc(C(C)(F)F)n1]

N-(1-(2-(1,1-Difluoroethyl)-6-ethylpyrimidin-4-yl)-3-(3-(dimethylamino)-3-methylpyrrolidin-1-yl)-1H-pyrazolo[4,3-c]pyridin-6-yl)acetamide was obtained, 4.0 g, 70.6% as a yellow solid, from N-(3-bromo-1-(2-(1,1-difluoroethyl)-6-ethylpyrimidin-4-yl)-1H-pyrazolo[4,3-c]pyridin-6-yl)acetamide (Preparation 393) and N,N,3-trimethylpyrrolidin-3-amine hydrochloride, following the procedure described in Example 268. This was further purified by SFC (Column:DAICEL CHIRALPAK AD (250 mm*30 mm,10 um); Mobile Phase: 35% of 0.1% NH₃H₂O EtOH; Flow Rate(mL/min): 80; Column temp: 35° C. to give

Peak 1, (R)—N-(1-(2-(1,1-difluoroethyl)-6-ethylpyrimidin-4-yl)-3-(3-(dimethylamino)-3-methylpyrrolidin-1-yl)-1H-pyrazolo[4,3-c]pyridin-6-yl)acetamide or (S)—N-(1-(2-(1,1-difluoroethyl)-6-ethylpyrimidin-4-yl)-3-(3-(dimethylamino)-3-methylpyrrolidin-1-yl)-1H-pyrazolo[4,3-c]pyridin-6-yl)acetamide (1.3 g, 33.3%) as a white solid. LCMS m/z=473.2 [M+H]⁺, ¹HNMR (400 MHz, CDCl₃) δ: ppm 9.48 (brs, 1H), 8.71 (s, 1H), 8.11 (s, 1H), 7.63 (s, 1H), 3.93-3.97 (m, 1H), 3.82-3.84 (m, 1H), 3.69 (s, 2H), 2.90 (q, J=8.0 Hz, 2H), 2.38 (s, 6H), 2.27-2.32 (m, 6H), 2.17-2.23 (m, 1H), 2.03-2.05 (m, 1H), 1.38 (t, J=8.0 Hz, 3H), 1.18 (s, 3H).

and Peak 2, (S)—N-(1-(2-(1,1-difluoroethyl)-6-ethylpyrimidin-4-yl)-3-(3-(dimethylamino)-3-methylpyrrolidin-1-yl)-1H-pyrazolo[4,3-c]pyridin-6-yl)acetamide or (R)—N-(1-(2-(1,1-difluoroethyl)-6-ethylpyrimidin-4-yl)-3-(3-(dimethylamino)-3-methylpyrrolidin-1-yl)-1H-pyrazolo[4,3-c]pyridin-6-yl)acetamide (1.05 g, 26.9%) as a white solid. LCMS m/z=473.2 [M+H]⁺, ¹HNMR (400 MHz, CDCl₃) δ: ppm 9.47 (brs, 1H), 8.71 (s, 1H), 8.16 (s, 1H), 7.62 (s, 1H), 3.92-3.95 (m, 1H), 3.82-3.84 (m, 1H), 3.69 (s, 2H), 2.90 (q, J=8.0 Hz, 2H), 2.38 (s, 6H), 2.27-2.33 (m, 6H), 2.17-2.24 (m, 1H), 2.02-2.05 (m, 1H), 1.38 (t, J=8.0 Hz, 3H), 1.19 (s, 3H).

### Examples 276 and 277: (S)—N-(3-(3-(diethylamino)-3-methylpyrrolidin-1-yl)-1-(2-(1,1-difluoroethyl)-6-ethylpyrimidin-4-yl)-1H-pyrazolo[4,3-c]pyridin-6-yl)acetamide and (R)—N-(3-(3-(diethylamino)-3-methylpyrrolidin-1-yl)-1-(2-(1,1-difluoroethyl)-6-ethylpyrimidin-4-yl)-1H-pyrazolo[4,3-c]pyridin-6-yl)acetamide

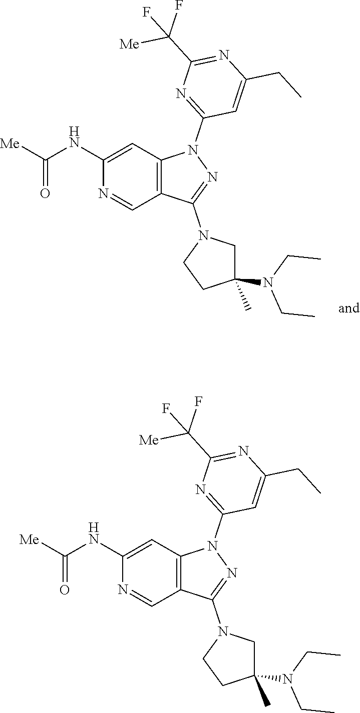 [SMILES: CCc1cc(-n2nc(N3CC[C@](C)(N(CC)CC)C3)c3cnc(NC(C)=O)cc32)nc(C(C)(F)F)n1 ; CCc1cc(-n2nc(N3CC[C@@](C)(N(CC)CC)C3)c3cnc(NC(C)=O)cc32)nc(C(C)(F)F)n1]

To a solution of N-(3-bromo-1-(2-(1,1-difluoroethyl)-6-ethylpyrimidin-4-yl)-1H-pyrazolo[4,3-c]pyridin-6-yl)acetamide (Preparation 393, 100 mg, 0.235 mmol) in DMSO (5.0 mL) was added N,N-diethyl-3-methylpyrrolidin-3-amine (Preparation 399, 73.5 mg, 0.470 mmol), CuI (9.0 mg, 0.047 mmol), K₃PO₄ (299.5 mg, 1.41 mmol) and 2,6-DFPAO (9.5 mg, 0.047 mmol). The reaction was stirred at 100° C. for 9 h under N₂. The mixture was purified by Prep-HPLC-C (gradient: 55% to 85% MeCN) to give N-(3-(3-(diethylamino)-3-methylpyrrolidin-1-yl)-1-(2-(1,1-difluoroethyl)-6-ethylpyrimidin-4-yl)-1H-pyrazolo[4,3-c]pyridin-6-yl)acetamide (60.0 mg, 51.0% yield) as a light-yellow solid. This was further purified by SFC (Column: DAICEL CHIRALPAK IG (250 mm*30 mm,10 um), Condition 45% (0.10% NH₃·H₂O MeOH); Flow Rate (ml/min): 80) to give Peak 1, (S)—N-(3-(3-(diethylamino)-3-methylpyrrolidin-1-yl)-1-(2-(1,1-difluoroethyl)-6-ethylpyrimidin-4-yl)-1H-pyrazolo[4,3-c]pyridin-6-yl)acetamide or (R)—N-(3-(3-(diethylamino)-3-methylpyrrolidin-1-yl)-1-(2-(1,1-difluoroethyl)-6-ethylpyrimidin-4-yl)-1H-pyrazolo[4,3-c]pyridin-6-yl)acetamide (25.5 mg, 42.5% yield) as a white solid. LCMS m/z=501.2 [M+H]^{+ 1}H NMR (400 MHz, CDCl₃) δ ppm: 9.48 (s, 1H), 8.71 (s, 1H), 8.06 (s, 1H), 7.63 (s, 1H), 3.70-3.94 (m, 4H), 2.91 (q, J=7.6 Hz, 2H), 2.45-2.85 (m, 4H), 2.23-2.33 (m, 7H), 2.00-2.04 (m, 1H), 1.38 (t, J=7.2 Hz, 3H), 1.18-1.25 (m, 9H).

and Peak 2, (R)—N-(3-(3-(diethylamino)-3-methylpyrrolidin-1-yl)-1-(2-(1,1-difluoroethyl)-6-ethylpyrimidin-4-yl)-1H-pyrazolo[4,3-c]pyridin-6-yl)acetamide or (S)—N-(3-(3-(diethylamino)-3-methylpyrrolidin-1-yl)-1-(2-(1,1-difluoroethyl)-6-ethylpyrimidin-4-yl)-1H-pyrazolo[4,3-c]pyridin-6-yl)acetamide (23.1 mg, 38.5% yield) as a white solid. LCMS m/z=501.2 [M+H]⁺; ¹H NMR: (400 MHz, CDCl₃) δ ppm: 9.48 (s, 1H), 8.71 (s, 1H), 8.06 (s, 1H), 7.62 (s, 1H), 3.71-3.95 (m, 4H), 2.70-2.94 (m, 6H), 2.23-2.33 (m, 7H), 2.00-2.05 (m, 1H), 1.38 (t, J=7.2 Hz, 3H), 0.99-1.25 (m, 9H).

### Examples 278 and 279: (S)—N-(3-(3-(diethylamino)-3-methylpyrrolidin-1-yl)-1-(2-(1,1-difluoroethyl)-6-methylpyrimidin-4-yl)-1H-pyrazolo[4,3-c]pyridin-6-yl)acetamide and (R)—N-(3-(3-(diethylamino)-3-methylpyrrolidin-1-yl)-1-(2-(1,1-difluoroethyl)-6-methylpyrimidin-4-yl)-1H-pyrazolo[4,3-c]pyridin-6-yl)acetamide

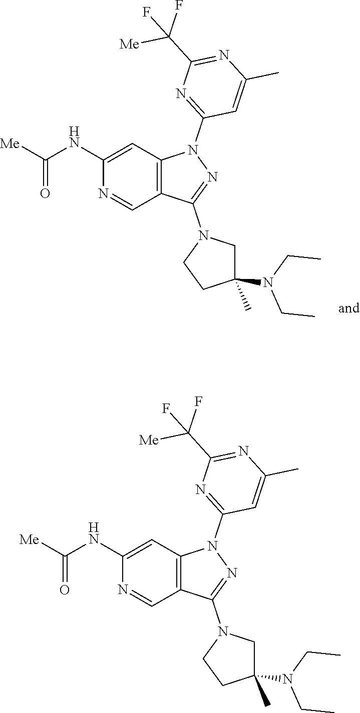 [SMILES: CCN(CC)[C@@]1(C)CCN(c2nn(-c3cc(C)nc(C(C)(F)F)n3)c3cc(NC(C)=O)ncc23)C1 ; CCN(CC)[C@]1(C)CCN(c2nn(-c3cc(C)nc(C(C)(F)F)n3)c3cc(NC(C)=O)ncc23)C1]

Zinc (63.6 mg, 0.973 mmol) was stirred with 2N HCl for 4 min, then washed with water, EtOH, and PE, and dried under high vacuum. To a solution of N-(3-bromo-1-(2-(1,1-difluoroethyl)-6-methylpyrimidin-4-yl)-1H-pyrazolo[4,3-c]pyridin-6-yl)acetamide (Preparation 391, 400 mg, 0.97 mmol) in DMPU (5 mL) was added N,N-diethyl-3-methylpyrrolidin-3-amine (Preparation 399, 207.6 mg, 1.46 mmol), Zinc (63.6 mg, 0.973 mmol), NiBr 1,2-dimethoxyethane (3.0 mg, 9.73 umol), DABCO (327.3 mg, 2.92 mmol) and DBU (296.2 mg, 1.95 mmol) at 25° C. The reaction was stirred at 55° C. for 16 h under N₂. The mixture was purified by Prep-HPLC-D (Gradient: 12% to 32% MeCN) to give N-(3-(3-(diethylamino)-3-methylpyrrolidin-1-yl)-1-(2-(1,1-difluoroethyl)-6-methylpyrimidin-4-yl)-1H-pyrazolo[4,3-c]pyridin-6-yl)acetamide (160 mg, 34.8% yield) as a white solid. This was further separated by SFC (Condition 40% (0.1% NH₃·H₂O EtOH) Column: DAICEL CHIRALPAK IG (250 mm*30 mm,10 um); Flow Rate (ml/min): 150) to give (S)—N-(3-(3-(diethylamino)-3-methylpyrrolidin-1-yl)-1-(2-(1,1-difluoroethyl)-6-methylpyrimidin-4-yl)-1H-pyrazolo[4,3-c]pyridin-6-yl)acetamide or (R)—N-(3-(3-(diethylamino)-3-methylpyrrolidin-1-yl)-1-(2-(1,1-difluoroethyl)-6-methylpyrimidin-4-yl)-1H-pyrazolo[4,3-c]pyridin-6-yl)acetamide (63.0 mg, 39.4% yield) as a white solid. LCMS m/z=473.2 [M+H]^{+ 1}H NMR: (400 MHz, CDCl₃) δ ppm 9.46 (s, 1H), 8.72 (s, 1H), 8.08 (s, 1H), 7.63 (s, 1H), 3.92-4.00 (m, 2H), 3.54-3.77 (m, 3H), 2.81 (br s, 4H), 2.64 (s, 3H), 2.24-2.33 (m, 7H), 2.17 (br s, 1H), 1.15 (t, J=7.2 Hz, 6H).

and (R)—N-(3-(3-(diethylamino)-3-methylpyrrolidin-1-yl)-1-(2-(1,1-difluoroethyl)-6-methylpyrimidin-4-yl)-1H-pyrazolo[4,3-c]pyridin-6-yl)acetamide or (S)—N-(3-(3-(diethylamino)-3-methylpyrrolidin-1-yl)-1-(2-(1,1-difluoroethyl)-6-methylpyrimidin-4-yl)-1H-pyrazolo[4,3-c]pyridin-6-yl)acetamide (65.0 mg, 40.6% yield) as a white solid. LCMS m/z=473.2 [M+H]⁺. ¹H NMR: (400 MHz, CDCl₃) δ ppm 9.44 (s, 1H), 8.70 (s, 1H), 8.16 (s, 1H), 7.62 (s, 1H), 3.92-4.00 (m, 2H), 3.64-3.73 (m, 3H), 2.81-2.84 (m, 4H), 2.64 (s, 3H), 2.27-2.33 (m, 7H), 2.23 (br s, 1H), 1.15 (t, J=7.2 Hz, 6H).

### Example 280: N-(1-(2-(1,1-difluoroethyl)-6-methylpyrimidin-4-yl)-3-((3R,4S)-3-(dimethylamino)-4-methylpyrrolidin-1-yl)-1H-pyrazolo[4,3-c]pyridin-6-yl)acetamide

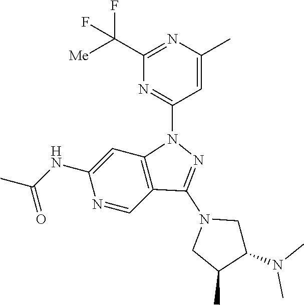 [SMILES: CC(=O)Nc1cc2c(cn1)c(N1C[C@H](C)[C@@H](N(C)C)C1)nn2-c1cc(C)nc(C(C)(F)F)n1]

To a solution of N-(3-((3R,4S)-3-amino-4-methylpyrrolidin-1-yl)-1-(2-(1,1-difluoroethyl)-6-methylpyrimidin-4-yl)-1H-pyrazolo[4,3-c]pyridin-6-yl)acetamide hydrochloride (Preparation 404, 120 mg, 0.279 mmol) and NaBH₃CN (87.6 mg, 1.39 mmol) in MeOH (10 mL) was added (CH₂O)ₙ(1.0 g, 0.836 mmol) and the reaction was stirred at 25° C. for 12 h. The mixture was purified by Prep-HPLC-C (Gradient: 30% to 60% MeCN) Column to give N-(1-(2-(1,1-difluoroethyl)-6-methylpyrimidin-4-yl)-3-((3R,4S)-3-(dimethylamino)-4-methylpyrrolidin-1-yl)-1H-pyrazolo[4,3-c]pyridin-6-yl)acetamide (68.0 mg, 53.2% yield) as a white solid. LCMS m/z=459.3 [M+H]^{+ 1}H NMR: (400 MHz, CDCl₃) δ ppm: 9.46 (s, 1H), 8.72 (s, 1H), 8.15 (s, 1H), 7.63 (s, 1H), 3.98-4.03 (m, 1H), 3.82-3.86 (m, 1H), 3.70-3.75 (m, 1H), 3.33-3.37 (m, 1H), 2.94-2.99 (m, 1H), 2.64 (s, 3H), 2.49-2.56 (m, 1H), 2.42 (s, 6H), 2.23-2.33 (m, 6H), 1.23 (d, J=6.4 Hz, 3H).

Examples 281 to 283: The compounds in the following table were obtained from the appropriate amine and (CH₂O)ₙ following the procedure described in Example 280.

| | Name, Structure | |
|---|---|---|
| Compound | Name, Structure, Starting materials | Starting Material (SM), |
| No | (SM), yield | Yield, Data |
| 281 | N-(1-(2-(1,1-difluoroethyl)-6- methylpyrimidin-4-yl)-3-((3R,4R)-3- (dimethylamino)-4-methylpyrrolidin-1-yl)- 1H-pyrazolo[4,3-c]pyridin-6-yl)acetamide 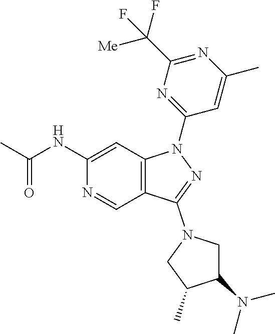 | LCMS m/z = 459.3 [M + H]⁺ ¹H NMR: (400 MHz, CDCl₃) δ ppm: 9.49 (s, 1H), 8.75 (s, 1H), 8.10 (s, 1H), 7.66 (s, 1H), 4.02-4.07 (m, 1H), 3.87- 3.92 (m, 1H), 3.84-3.77 (m, 1H), 3.37-3.40 (m, 1H), 3.04- 3.07 (m, 1H), 2.67 (s, 3H), 2.58-2.64 (m, 1H), 2.50 (s, 6H), 2.26-2.37 (m, 6H), 1.28 (d, J = 6.8 Hz, 3H) |
| | SM: N-(3-((3S,4R)-3-amino-4- | |
| | methylpyrrolidin-1-yl)-1-(2-(1,1- | |
| | difluoroethyl)-6-methylpyrimidin-4-yl)-1H- | |
| | pyrazolo[4,3-c]pyridin-6-yl)acetamide | |
| | hydrochloride (Preparation 405) | |
| | White solid, 26.3 mg, 30.8% yield | |
| 282 | N-(1-(2-(1,1-difluoroethyl)-6- methylpyrimidin-4-yl)-3-((3S,4S)-3- (dimethylamino)-4-methylpyrrolidin-1-yl)- 1H-pyrazolo[4,3-c]pyridin-6-yl)acetamide 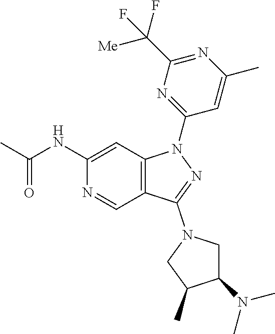 [SMILES: CC(=O)Nc1cc2c(cn1)c(N1C[C@@H](N(C)C)[C@@H](C)C1)nn2-c1cc(C)nc(C(C)(F)F)n1] | LCMS m/z = 459.2 [M + H]⁺ ¹H NMR: (400 MHz, CDCl₃) δ ppm: 9.47 (s, 1H), 8.71 (s, 1H), 8.16 (s, 1H), 7.63 (s, 1H), 3.86-3.90 (m, 2H), 3.59- 3.64 (m, 2H), 2.73-2.79 (m, 1H), 2.64 (s, 3H), 2.53-2.55 (m, 1H), 2.35 (s, 6H), 2.22- 2.28 (m, 6H), 1.12 (d, J = 7.2 Hz, 3H). |
| | SM: N-(3-((3S,4S)-3-amino-4- | |
| | methylpyrrolidin-1-yl)-1-(2-(1,1- | |
| | difluoroethyl)-6-methylpyrimidin-4-yl)-1H- | |
| | pyrazolo[4,3-c]pyridin-6-yl)acetamide | |
| | hydrochloride (Preparation 406) | |
| | 124.0 mg, 56.4% yield as a white solid. | |
| 283 | N-(1-(2-(1,1-difluoroethyl)-6- methylpyrimidin-4-yl)-3-((3R,4R)-3- (dimethylamino)-4-methylpyrrolidin-1-yl)- 1H-pyrazolo[4,3-c]pyridin-6-yl)acetamide 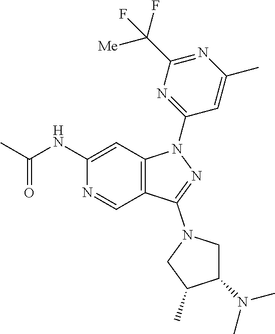 [SMILES: CC(=O)Nc1cc2c(cn1)c(N1C[C@@H](C)[C@@H](N(C)C)C1)nn2-c1cc(C)nc(C(C)(F)F)n1] | CMS m/z = 459.2 [M + H]⁺ ¹H NMR (500 MHz, CDCl₃) δ ppm 9.48 (s, 1H), 8.71 (s, 1H), 8.05 (s, 1H), 7.64 (s, 1H), 3.89-3.91 (m, 2H), 3.64- 3.66 (m, 2H), 2.77-2.93 (m, 1H), 2.64 (s, 3H), 2.58 (br s, 1H), 2.40 (br s, 6H), 2.25- 2.32 (m, 6H), 1.17 (br s, 3H). |
| | SM: N-(3-((3R,4R)-3-amino-4- | |
| | methylpyrrolidin-1-yl)-1-(2-(1,1- | |
| | difluoroethyl)-6-methylpyrimidin-4-yl)-1H- | |
| | pyrazolo[4,3-c]pyridin-6-yl)acetamide | |
| | hydrochloride (Preparation 407) | |
| | 108 mg, 26.5% yield) as a white solid. | |

### Example 284: N-(3-(6-oxa-3-azabicyclo[3.1.1]heptan-3-yl)-1-(2-(1,1-difluoroethyl)-6-ethylpyrimidin-4-yl)-1H-pyrazolo[4,3-c]pyridin-6-yl)acetamide

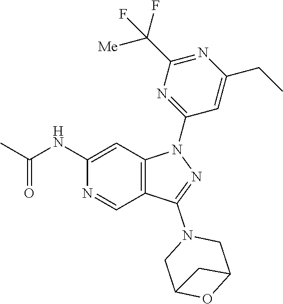 [SMILES: CCc1cc(-n2nc(N3CC4CC(C3)O4)c3cnc(NC(C)=O)cc32)nc(C(C)(F)F)n1]

To a solution of N-(3-(6-oxa-3-azabicyclo[3.1.1]heptan-3-yl)-1H-pyrazolo[4,3-c]pyridin-6-yl)acetamide (Preparation 410, 153.0 mg, 0.56 mmol) in dioxane (5.0 mL) was added 4-chloro-2-(1,1-difluoroethyl)-6-ethylpyrimidine (Preparation 390, 138.8 mg, 0.672 mmol), Pd₂(dba)₃ (51.3 mg, 0.056 mmol), K₃PO₄ (356.5 mg, 1.68 mmol) and Xantphos (64.8 mg, 0.112 mmol) and the reaction was stirred at 100° C. for 2 h under N₂. The reaction mixture was concentrated in vacuo and was purified by prep-HPLC-C (gradient: 30 to 60% MeCN) to give N-(3-(6-oxa-3-azabicyclo[3.1.1]heptan-3-yl)-1-(2-(1,1-difluoroethyl)-6-ethylpyrimidin-4-yl)-1H-pyrazolo[4,3-c]pyridin-6-yl)acetamide (106.1 mg, 42.7% yield) as a white solid. LCMS m/z=444.1 [M+H]^{+ 1}H NMR: (500 MHz, CDCl₃) δ ppm: 9.53 (s, 1H), 8.82 (s, 1H), 8.15 (s, 1H), 7.65 (s, 1H), 4.84 (d, J=6.0 Hz, 2H), 4.08-4.15 (m, 2H), 3.98-4.01 (m, 2H), 3.36-3.39 (m, 1H), 2.91 (q, J=7.5 Hz, 2H), 2.25-2.33 (m, 6H), 2.14 (d, J=9.0 Hz, 1H), 1.38 (t, J=7.5 Hz, 3H).

### Example 285: N-(1-(6-(1,1-difluoroethyl)pyridin-2-yl)-3-(6-ethyl-3,6-diazabicyclo[3.1.1]heptan-3-yl)-1H-pyrazolo[4,3-c]pyridin-6-yl)acetamide

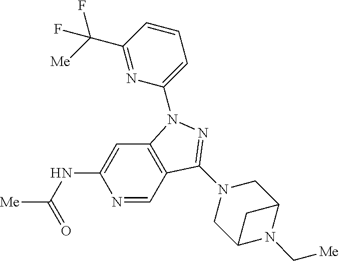 [SMILES: CCN1C2CC1CN(c1nn(-c3cccc(C(C)(F)F)n3)c3cc(NC(C)=O)ncc13)C2]

Step 1: To a solution of tert-butyl 3-(6-acetamido-1-(6-(1,1-difluoroethyl)pyridin-2-yl)-1H-pyrazolo[4,3-c]pyridin-3-yl)-3,6-diazabicyclo[3.1.1]heptane-6-carboxylate (Preparation 512, 170 mg, 0.331 mmol) in DCM (3 mL) was added HCl/dioxane (4 M, 1 mL) and the reaction was stirred at 20° C. for 2 h. The mixture was concentrated to give N-(3-(3,6-diazabicyclo[3.1.1]heptan-3-yl)-1-(6-(1,1-difluoroethyl)pyridin-2-yl)-1H-pyrazolo[4,3-c]pyridin-6-yl)acetamide hydrochloride (150 mg, crude) as yellow oil. LCMS m/z=414.2 [M+H]⁺.

Step 2: To a solution of N-(3-(3,6-diazabicyclo[3.1.1]heptan-3-yl)-1-(6-(1,1-difluoroethyl)pyridin-2-yl)-1H-pyrazolo[4,3-c]pyridin-6-yl)acetamide hydrochloride (150 mg, 0.33 mmol) and acetaldehyde in MeOH (3 mL) was added NaBH₃CN (104.8 mg, 1.67 mmol) and the reaction was stirred at 25° C. for 2 h. The mixture was concentrated and extracted with DCM (100 mL×2). The combined organic layers were washed with brine (50 mL), dried over anhydrous Na₂SO₄, filtered and concentrated under reduced pressure to give a residue, which was purified by prep-HPLC-C (40% to 60% MeCN) to give N-(1-(6-(1,1-difluoroethyl)pyridin-2-yl)-3-(6-ethyl-3,6-diazabicyclo[3.1.1]heptan-3-yl)-1H-pyrazolo[4,3-c]pyridin-6-yl)acetamide (25 mg, 17.0% yield) as a white solid. LCMS m/z=442.3 [M+H]⁺. 1H NMR: (500 MHz, DMSO-d₆) δ ppm 10.69 (s, 1H), 9.35 (s, 1H), 9.04 (s, 1H), 8.09 (d, J=8.0 Hz, 1H), 7.95 (d, J=8.0 Hz, 1H), 7.48 (d, J=7.0 Hz, 1H), 3.93 (br d, J=11.0 Hz, 2H), 3.75-3.69 (m, 4H), 2.52-2.49 (m, 1H), 2.42 (q, J=7.0 Hz, 2H), 2.26 (t, J=19.5 Hz, 3H), 2.15 (s, 3H), 1.66 (d, J=8.5 Hz, 1H), 0.95 (t, J=7.0 Hz, 3H).

### Example 286: (S)—N-(3-(3-(diethylamino)pyrrolidin-1-yl)-1-(6-(2-fluoropropan-2-yl)pyrazin-2-yl)-1H-pyrazolo[4,3-c]pyridin-6-yl)acetamide

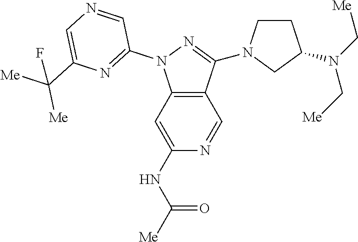 [SMILES: CCN(CC)[C@H]1CCN(c2nn(-c3cncc(C(C)(C)F)n3)c3cc(NC(C)=O)ncc23)C1]

To a solution of N-(3-bromo-1-(6-(2-fluoropropan-2-yl)pyrazin-2-yl)-1H-pyrazolo[4,3-c]pyridin-6-yl)acetamide (Preparation 500, 100 mg, 0.254 mmol) in DMSO (4 mL) was added (S)—N,N-diethylpyrrolidin-3-amine (108.5 mg, 0.763 mmol), K₃PO₄ (162.0 mg, 0.763 mmol), CuI (4.8 mg, 0.025 mmol) and 2,6-DFPAO (15.3 mg, 0.076 mmol). The reaction was stirred at 110° C. for 4 h under N₂ under microwave irradiation. The mixture was concentrated and purified by Prep-HPLC-U (10% to 40% MeCN) to give (S)—N-(3-(3-(diethylamino)pyrrolidin-1-yl)-1-(6-(2-fluoropropan-2-yl)pyrazin-2-yl)-1H-pyrazolo[4,3-c]pyridin-6-yl)acetamide (50 mg, 43.3% yield) as a yellow solid. LCMS m/z=455.2 [M+H]⁺. ¹HNMR (400 MHz, DMSO-d₆) δ: ppm 10.69 (s, 1H), 9.28 (s, 1H), 9.02 (s, 1H), 8.92 (s, 1H), 8.51 (s, 1H), 8.17 (br s, 1H), 3.91-3.83 (m, 2H), 3.72-3.61 (m, 3H), 2.68-2.63 (m, 4H), 2.20-2.15 (m, 4H), 1.99-1.82 (m, 7H), 1.00 (t, J=7.2 Hz, 6H).

Examples 287 to 299: The compounds in the following table were prepared from the appropriate bromide and amine, following a similar procedure to that described in Example 286.

Bromo pyrazolo[4,3-c]pyridine 1: N-(3-bromo-1-(6-(2-fluoropropan-2-yl)pyrazin-2-yl)-1H-pyrazolo[4,3-c]pyridin-6-yl)acetamide (Preparation 500); Bromo pyrazolo[4,3-c]pyridine 2: N-(3-bromo-1-(2-(2-fluoropropan-2-yl)-6-methylpyrimidin-4-yl)-1H-pyrazolo[4,3-c]pyridin-6-yl)acetamide (Preparation 501); Bromo pyrazolo[4,3-c]pyridine 3: N-(3-bromo-1-(6-(1,1-difluoroethyl)pyrazin-2-yl)-1H-pyrazolo[4,3-c]pyridin-6-yl)acetamide (Preparation 504); Bromo pyrazolo[4,3-c]pyridine 4: N-(3-bromo-1-(2-(1,1-difluoroethyl)-6-ethylpyrimidin-4-yl)-1H-pyrazolo[4,3-c]pyridin-6-yl)acetamide (Preparation 393); Bromo pyrazolo[4,3-c]pyridine 5: N-(3-bromo-1-(2-(1,1-difluoroethyl)-6-isopropylpyrimidin-4-yl)-1H-pyrazolo[4,3-c]pyridin-6-yl)acetamide (Preparation 508); Bromo pyrazolo[4,3-c]pyridine 6: N-(3-bromo-1-(6-(1,1-difluoroethyl)-4-methoxypyridin-2-yl)-1H-pyrazolo[4,3-c]pyridin-6-yl)acetamide (Preparation 509); Bromo pyrazolo[4,3-c]pyridine 7: N-(3-bromo-1-(6-(1,1-difluoroethyl)-4-methylpyridin-2-yl)-1H-pyrazolo[4,3-c]pyridin-6-yl)acetamide (Preparation 507)

| Example No | Name/Structure/Starting Materials (SM)/Data |
|---|---|
| 287 | (R)-N-(3-(3-(diethylamino)pyrrolidin-1-yl)-1-(6-(2-fluoropropan-2- |
| | yl)pyrazin-2-yl)-1H-pyrazolo[4,3-c]pyridin-6-yl)acetamide |
| | 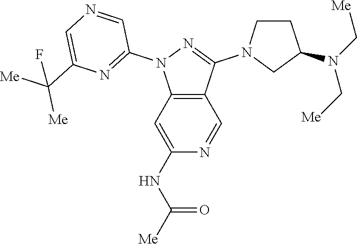 [SMILES: CCN(CC)[C@@H]1CCN(c2nn(-c3cncc(C(C)(C)F)n3)c3cc(NC(C)=O)ncc23)C1] |
| | SM: (S)-N,N-diethylpyrrolidin-3-amine and Bromo pyrazolo[4,3-c]pyridine |
| | 1 |
| | prep-HPLC-Q (10% to 40% MeCN). 41.6 mg, 36.0% yield as a yellow solid. |
| | LCMS m/z = 455.3 [M + H]⁺. 1H NMR (400 MHz, DMSO-d₆) δ ppm: 10.69 |
| | (s, 1H), 9.29 (s, 1H), 9.02 (s, 1H), 8.93 (s, 1H), 8.51 (s, 1H), 3.94-3.86 (m, |
| | 2H), 3.66-3.65 (m, 1H), 3.42-3.40 (m, 2H), 2.69-2.64 (m, 4H), 2.20-2.19 (m, |
| | 1H), 2.15 (s, 3H), 1.91-1.90 (m, 1H), 1.87 (s, 3H), 1.82 (s, 3H), 1.01 (t, J = |
| | 7.2 Hz, 6H). |
| 288 | (R)-N-(3-([1,3′-bipyrrolidin]-1′-yl)-1-(6-(2-fluoropropan-2-yl)pyrazin-2-yl)- |
| | 1H-pyrazolo[4,3-c]pyridin-6-yl)acetamide |
| | 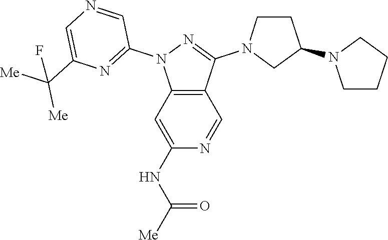 [SMILES: CC(=O)Nc1cc2c(cn1)c(N1CC[C@@H](N3CCCC3)C1)nn2-c1cncc(C(C)(C)F)n1] |
| | SM: (R)-1,3′-bipyrrolidine hydrochloride and Bromo pyrazolo[4,3-c]pyridine |
| | 1 |
| | Prep-HPLC-C (40% to 70% MeCN) |
| | 25 mg, 21.7% yield as a yellow solid. LCMS m/z = 453.3 [M + H]⁺ |
| | 1H NMR: (400 MHz, CDCl₃) δ ppm: 9.30 (s, 1H), 9.13 (s, 1H), 8.73 (s, 1H), |
| | 8.58 (s, 1H), 8.05 (s, 1H), 3.99-3.94 (m, 2H), 3.80-3.76 (m, 1H), 3.70-3.68 |
| | (m, 1H), 3.12-3.08 (m, 1H), 2.78-2.69 (m, 4H), 2.35-2.32 (m, 4H), 2.30-2.26 |
| | (m, 1H), 1.95 (s, 3H), 1.92-1.89 (m, 7H). |
| 289 | (S)-N-(3-([1,3′-bipyrrolidin]-1′-yl)-1-(6-(2-fluoropropan-2-yl)pyrazin-2-yl)- |
| | 1H-pyrazolo[4,3-c]pyridin-6-yl)acetamide |
| | 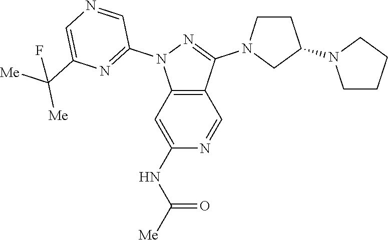 [SMILES: CC(=O)Nc1cc2c(cn1)c(N1CC[C@H](N3CCCC3)C1)nn2-c1cncc(C(C)(C)F)n1] |
| | SM: (S)-1,3′-bipyrrolidine hydrochloride and Bromo pyrazolo[4,3-c]pyridine |
| | 1 |
| | Prep-HPLC-C (41% to 71% MeCN). 42 mg, 30.4% yield as a yellow solid. |
| | LCMS m/z = 453.3 [M + H]⁺. 1H NMR: (400 MHz, CDCl₃) δ ppm 9.30 (s, |
| | 1H), 9.12 (s, 1H), 8.73 (s, 1H), 8.57 (s, 1H), 8.19 (s, 1H), 3.97-3.91 (m, 2H), |
| | 3.79-3.71 (m, 1H), 3.64-3.59 (m, 1H), 3.05-2.96 (m, 1H), 2.67 (br s, 4H), |
| | 2.28-2.23 (m, 4H), 2.17-2.10 (m, 1H), 1.94 (s, 3H), 1.89-1.85 (m, 7H) |
| 290 | N-(3-(3-(dimethylamino)-3-methylpyrrolidin-1-yl)-1-(6-(2-fluoropropan-2- |
| | yl)pyrazin-2-yl)-1H-pyrazolo[4,3-c]pyridin-6-yl)acetamide |
| | 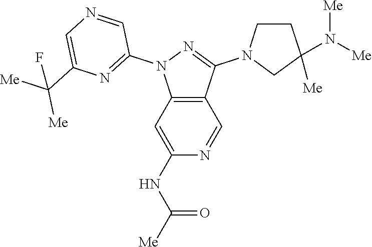 [SMILES: CC(=O)Nc1cc2c(cn1)c(N1CCC(C)(N(C)C)C1)nn2-c1cncc(C(C)(C)F)n1] |
| | SM: N,N,3-trimethylpyrrolidin-3-amine hydrochloride and Bromo |
| | pyrazolo[4,3-c]pyridine 1 |
| | prep-HPLC-S (30% to 60% MeCN). 18.0 mg, 20.0% yield as a green solid. |
| | LCMS m/z = 441.3 [M + H]⁺ 1H NMR: (400 MHz, DMSO-d₆) δ ppm: 10.68 |
| | (s, 1H), 9.26 (s, 1H), 8.99 (s, 1H), 8.89 (s, 1H), 8.49 (s, 1H), 3.85-3.81 (m, |
| | 1H), 3.75-3.72 (m, 1H), 3.66-3.63 (m, 1H), 3.52-3.48 (m, 1H), 2.22 (s, 6H), |
| | 2.15 (s, 3H), 1.99-1.94 (m, 2H), 1.86 (s, 3H), 1.80 (s, 3H), 1.05 (s, 3H). |
| 291 | N-(1-(6-(2-fluoropropan-2-yl)pyrazin-2-yl)-3-(3-(isopropylamino)pyrrolidin- |
| | 1-yl)-1H-pyrazolo[4,3-c]pyridin-6-yl)acetamide |
| | 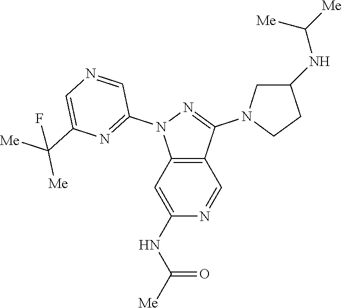 [SMILES: CC(=O)Nc1cc2c(cn1)c(N1CCC(NC(C)C)C1)nn2-c1cncc(C(C)(C)F)n1] |
| | SM: Bromo pyrazolo[4,3-c]pyridine 1 and N-isopropylpyrrolidin-3-amine |
| | Prep-HPLC-C (44% to 64% MeCN). 11.8 mg, 13.2% yield as a white solid. |
| | LCMS m/z = 441.2 [M + H]⁺ ¹H NMR: (500 MHz, CDCl₃) δ ppm: 9.31 (s, 1H), |
| | 9.13 (s, 1H), 8.78-8.71 (m, 1H), 8.58 (s, 1H), 8.13-8.10 (m, 1H), 3.99-3.99 (m, |
| | 2H), 3.76-3.69 (m, 2H), 3.48 (m, 1H), 3.02-2.99 (m, 1H), 2.34 (m, 1H), 2.26 |
| | (s, 3H), 1.94 (s, 3H), 1.90 (s, 3H), 1.79-1.71 (m, 1H), 1.16-1.14 (m, 6H). |
| 292 | N-(3-(3-(dimethylamino)-3-methylpyrrolidin-1-yl)-1-(2-(2-fluoropropan-2- |
| | yl)-6-methylpyrimidin-4-yl)-1H-pyrazolo[4,3-c]pyridin-6-yl)acetamide |
| | 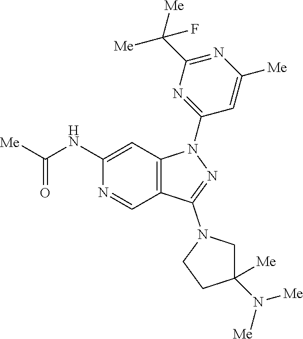 [SMILES: CC(=O)Nc1cc2c(cn1)c(N1CCC(C)(N(C)C)C1)nn2-c1cc(C)nc(C(C)(C)F)n1] |
| | SM: N,N,3-trimethylpyrrolidin-3-amine hydrochloride and Bromo |
| | pyrazolo[4,3-c]pyridine 2 |
| | Prep-HPLC-C (33% to 63% MeCN) 38 mg, 34% yield as a white solid. LCMS |
| | m/z = 445.2 [M + H]⁺ ¹H NMR: (400 MHz, CDCl₃) δ ppm: 9.44 (s, 1H), 8.72 |
| | (br s, 1H), 8.05 (s, 1H), 7.52 (s, 1H), 3.96-3.92 (m, 1H), 3.86-3.81 (m, 1H), |
| | 3.68 (s, 2H), 2.62 (s, 3H), 2.37 (s, 6H), 2.27 (s, 3H), 2.21-2.18 (m, 1H), 2.07- |
| | 2.02 (m, 1H), 1.98 (s, 3H), 1.93 (s, 3H), 1.17 (s, 3H) |
| 293 | (S)-N-(3-([1,3′-bipyrrolidin]-1′-yl)-1-(6-(1,1-difluoroethyl)pyrazin-2-yl)-1H- |
| | pyrazolo[4,3-c]pyridin-6-yl)acetamide |
| | 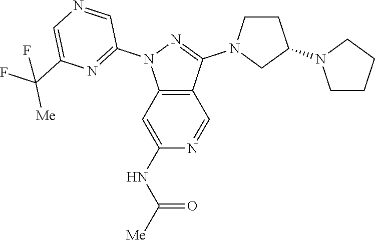 [SMILES: CC(=O)Nc1cc2c(cn1)c(N1CC[C@H](N3CCCC3)C1)nn2-c1cncc(C(C)(F)F)n1] |
| | SM: (S)-1,3′-bipyrrolidine hydrochloride and Bromo pyrazolo[4,3-c]pyridine |
| | 3 |
| | Prep-HPLC-C (40% to 70% MeCN). 28 mg, 24.4% yield as a yellow solid. |
| | LCMS m/z = 457.2 [M + H]⁺ 1H NMR: (400 MHz, CDCl₃) δ ppm 9.32 (s, |
| | 1H), 9.29 (s, 1H), 8.74 (s, 1H), 8.67 (s, 1H), 8.06 (s, 1H), 3.98-3.94 (m, 2H), |
| | 3.81-3.74 (m, 1H), 3.68 (br s, 1H), 3.08 (br s, 1H), 2.73 (br s, 4H), 2.37-2.25 |
| | (m, 8H), 1.90 (br s, 4H) |
| 294 | (R)-N-(3-([1,3′-bipyrrolidin]-1′-yl)-1-(6-(1,1-difluoroethyl)pyrazin-2-yl)-1H- |
| | pyrazolo[4,3-c]pyridin-6-yl)acetamide |
| | 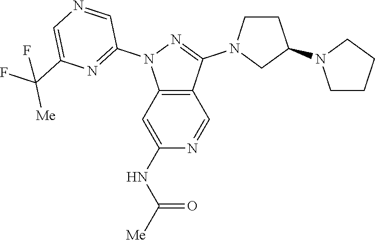 [SMILES: CC(=O)Nc1cc2c(cn1)c(N1CC[C@@H](N3CCCC3)C1)nn2-c1cncc(C(C)(F)F)n1] |
| | SM: (R)-1,3′-bipyrrolidine hydrochloride and Bromo pyrazolo[4,3-c]pyridine |
| | 3. |
| | Prep-HPLC-C (40% to 70% MeCN). 38 mg, 27.6% yield as a yellow solid. |
| | LCMS m/z = 457.3 [M + H]⁺. 1H NMR: (400 MHz, CDCl₃) δ ppm: 9.32 (s, |
| | 1H), 9.29 (s, 1H), 8.74 (s, 1H), 8.66 (s, 1H), 8.08 (s, 1H), 3.97-3.93 (m, 2H), |
| | 3.77-3.75 (m, 1H), 3.64-3.59 (m, 1H), 3.02-2.99 (m, 1H), 2.70-2.65 (m, 4H), |
| | 2.36-2.26 (m, 8H), 1.88-1.85 (m, 4H). |
| 295 | N-(1-(2-(1,1-difluoroethyl)-6-ethylpyrimidin-4-yl)-3-(3-(ethylamino)-3- |
| 78-3 | methylpyrrolidin-1-yl)-1H-pyrazolo[4,3-c]pyridin-6-yl)acetamide |
| | 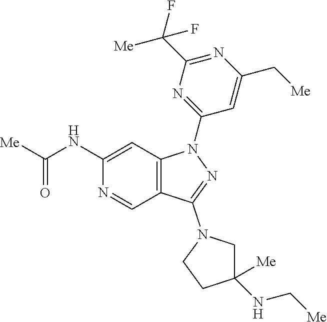 [SMILES: CCNC1(C)CCN(c2nn(-c3cc(CC)nc(C(C)(F)F)n3)c3cc(NC(C)=O)ncc23)C1] |
| | SM: Bromo pyrazolo[4,3-c]pyridine 4 and N-ethyl-3-methylpyrrolidin-3- |
| | amine |
| | prep-HPLC-C (36 to 65% MeCN). 65.0 mg, yield: 29.2% as a white solid. |
| | LCMS m/z = 473.3 [M + H]⁺ 1H NMR: (500 MHz, CDCl₃) δ ppm : 9.48 (s, |
| | 1H), 8.72 (s, 1H), 8.12 (s, 1H), 7.62 (s, 1H), 3.94-3.89 (m, 1H), 3.84-3.80 |
| | (m, 1H), 3.69 (d, J = 10.0 Hz, 1H), 3.59 (d, J = 10.0 Hz, 1H), 2.92-2.87 (m, |
| | 2H), 2.77-2.71 (m, 2H), 2.62 (s, 1H), 2.32 (s, 1H), 2.27-2.20 (m, 5H), 2.16- |
| | 2.13 (m, 1H), 2.03-1.97 (m, 1H), 1.40-1.36 (m, 6H), 1.16 (t, J = 7.0 Hz, 3H). |
| 296^{A} | N-(1-(2-(1,1-difluoroethyl)-6-isopropylpyrimidin-4-yl)-3-(3- |
| | (dimethylamino)-3-ethylpyrrolidin-1-yl)-1H-pyrazolo[4,3-c]pyridin-6- |
| | yl)acetamide |
| | 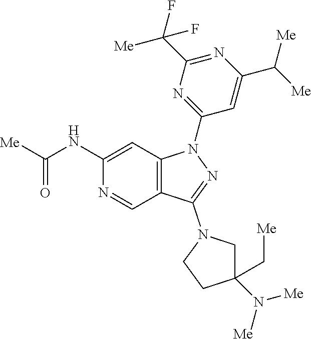 [SMILES: CCC1(N(C)C)CCN(c2nn(-c3cc(C(C)C)nc(C(C)(F)F)n3)c3cc(NC(C)=O)ncc23)C1] |
| | SM: Bromo pyrazolo[4,3-c]pyridine 5 and 3-ethyl-N,N-dimethylpyrrolidin- |
| | 3-amine |
| | Prep-HPLC-O (25% to 55% MeCN). 13 mg, 17.4% yield as a white solid. |
| | LCMS m/z = 501.3 [M + H]⁺. ¹H NMR (400 MHz, CDCl₃) δ ppm: 9.51 (s, 1H), |
| | 8.73 (s, 1H), 8.04 (s, 1H), 7.62 (s, 1H), 3.88-3.94 (m, 3H), 3.72-3.70 (m, 1H), |
| | 3.17-3.10 (m, 1H), 2.53 (br s, 6H), 2.33-2.21 (m, 7H), 2.11 (br s, 1H), 1.84 (br |
| | s, 2H), 1.38 (d, J-6.8 Hz, 6H), 1.08 (br s, 3H) |
| 297 | N-(1-(2-(1,1-difluoroethyl)-6-isopropylpyrimidin-4-yl)-3-(3- |
| | (ethyl(methyl)amino)-3-methylpyrrolidin-1-yl)-1H-pyrazolo[4,3-c]pyridin-6- |
| | yl)acetamide |
| | 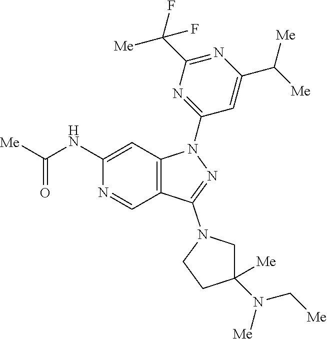 [SMILES: CCN(C)C1(C)CCN(c2nn(-c3cc(C(C)C)nc(C(C)(F)F)n3)c3cc(NC(C)=O)ncc23)C1] |
| | SM: Bromo pyrazolo[4,3-c]pyridine 5 |
| | HPLC-C (Gradient: 45 to 75% MeCN). 29.6 mg, 26.0% yield as a white |
| | solid. LCMS m/z = 501.3 [M + H]⁺ ¹H NMR: (500 MHz, CDCl₃) δ ppm: 9.49 |
| | (br s, 1H), 8.71 (s, 1H), 8.08 (s, 1H), 7.61 (s, 1H), 3.97-3.92 (m, 1H), 3.83- |
| | 3.81 (m, 1H), 3.74-3.72 (m, 2H), 3.15-3.10 (m, 1H), 2.73-2.50 (m, 2H), |
| | 2.36-2.23 (m, 10H), 2.08-2.04 (m, 1H), 1.37 (d, J = 7.0 Hz, 3H), 1.20 (s, 6H). |
| 298^{A} | N-(1-(6-(1,1-difluoroethyl)-4-methoxypyridin-2-yl)-3-(3- |
| | (isopropylamino)pyrrolidin-1-yl)-1H-pyrazolo[4,3-c]pyridin-6-yl)acetamide |
| | 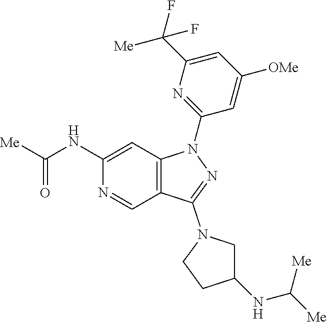 [SMILES: COc1cc(-n2nc(N3CCC(NC(C)C)C3)c3cnc(NC(C)=O)cc32)nc(C(C)(F)F)c1] |
| | SM: Bromo pyrazolo[4,3-c]pyridine 6 and 3-(isopropylamino)pyrrolidine |
| | prep-HPLC-C (43% to 73% MeCN). 13.08 mg, 13.9 % yield as a white solid. |
| | LCMS m/z = 474.2 [M + H]⁺ ¹H NMR: (400 MHz, CDCl₃) δ ppm : 9.30 (s, |
| | 1H), 8.64 (s, 1H), 7.98 (s, 1H), 7.35 (d, J = 1.6 Hz, 1H), 6.92 (d, J-2.0 Hz, 1H), |
| | 3.92-3.89 (m, 4H), 3.83-3.81 (m, 1H), 3.69-3.59 (m, 2H), 3.41-3.37 (m, 1H), |
| | 2.95-2.92 (m, 1H), 2.27-2.26 (m, 1H), 2.24-2.17 (m, 6H), 1.88-1.83 (m, 1H), |
| | 1.07 (t, J = 5.2 Hz, 6H). |
| 299 | N-(1-(6-(1,1-difluoroethyl)-4-methylpyridin-2-yl)-3-(3- |
| | (isopropylamino)pyrrolidin-1-yl)-1H-pyrazolo[4,3-c]pyridin-6-yl)acetamide |
| | 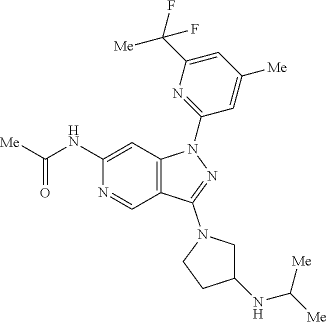 [SMILES: CC(=O)Nc1cc2c(cn1)c(N1CCC(NC(C)C)C1)nn2-c1cc(C)cc(C(C)(F)F)n1] |
| | SM: Bromo pyrazolo[4,3-c]pyridine 7 and 3-(isopropylamino)pyrrolidine |
| | HPLC-C (45% to 74% MeCN). 30.2 mg, 27% yield as a white solid. ¹H |
| | NMR (400 MHz, CDCl₃) δ ppm: 9.36 (s, 1H), 8.71 (s, 1H), 8.03 (s, 1H), |
| | 7.74 (s, 1H), 4.01-3.97 (m, 2H), 3.90-3.73 (m, 2H), 3.71-3.50 (m, 1H), 3.03- |
| | 3.02 (m, 1H), 2.47 (s, 3H), 2.34-2.25 (m, 7H), 1.96-1.95 (m, 1H), 1.15 (s, |
| | 6H). |

A-reaction stirred at 100° C. for 12 h, not under microwave irradiation

### Examples 300 and 301: (S)—N-(1-(6-(1,1-difluoroethyl)pyrazin-2-yl)-3-(3-(dimethylamino)-3-methylpyrrolidin-1-yl)-1H-pyrazolo[4,3-c]pyridin-6-yl)acetamide and (R)—N-(1-(6-(1,1-difluoroethyl)pyrazin-2-yl)-3-(3-(dimethylamino)-3-methylpyrrolidin-1-yl)-1H-pyrazolo[4,3-c]pyridin-6-yl)acetamide

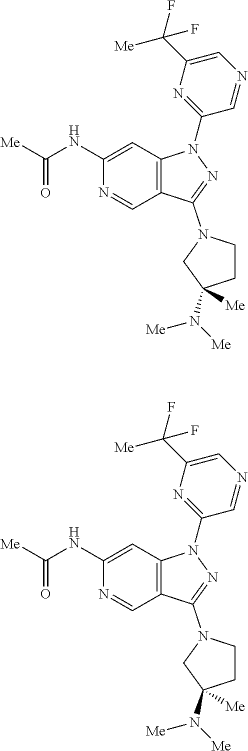 [SMILES: CC(=O)Nc1cc2c(cn1)c(N1CC[C@](C)(N(C)C)C1)nn2-c1cncc(C(C)(F)F)n1 ; CC(=O)Nc1cc2c(cn1)c(N1CC[C@@](C)(N(C)C)C1)nn2-c1cncc(C(C)(F)F)n1]

To a solution of N-(3-bromo-1-(6-(1,1-difluoroethyl)pyrazin-2-yl)-1H-pyrazolo[4,3-c]pyridin-6-yl)acetamide (Preparation 504, 200 mg, 0.503 mmol) in DMSO (10 mL) was added N,N,3-trimethylpyrrolidin-3-amine dihydrochloride (193.7 mg, 1.51 mmol), K₃PO₄ (320.7 mg, 1.51 mmol), 2,6-DFPAO (40.5 mg, 0.201 mmol) and CuI (19.2 mg, 0.101 mmol). The resulting mixture was stirred at 130° C. for 16 h under N₂. The mixture was concentrated and purified by Prep-HPLC-L (37% to 67% MeCN) to give N-(1-(6-(1,1-difluoroethyl)pyrazin-2-yl)-3-(3-(dimethylamino)-3-methylpyrrolidin-1-yl)-1H-pyrazolo[4,3-c]pyridin-6-yl)acetamide, 120 mg, 53.6% yield as a yellow solid. This was further purified by SFC (Column:DAICEL CHIRALPAK AD (250 mm*30 mm, 10 μm); Mobile Phase 35% (0.1% NH₃·H₂O EtOH) at 60 mL/min to give Peak 1, enantiomer 1, (S)—N-(1-(6-(1,1-difluoroethyl)pyrazin-2-yl)-3-(3-(dimethylamino)-3-methylpyrrolidin-1-yl)-1H-pyrazolo[4,3-c]pyridin-6-yl)acetamide or (R)—N-(1-(6-(1,1-difluoroethyl)pyrazin-2-yl)-3-(3-(dimethylamino)-3-methylpyrrolidin-1-yl)-1H-pyrazolo[4,3-c]pyridin-6-yl)acetamide (33 mg, 27.5% yield) as a yellow solid. LCMS m/z=445.3 [M+H]⁺. ¹H NMR (400 MHz, CDCl₃) δ: ppm 9.33 (s, 1H), 9.28 (s, 1H), 8.73 (d, J=0.8 Hz, 1H), 8.66 (s, 1H), 8.10 (s, 1H), 3.98-3.91 (m, 1H), 3.87-3.79 (m, 1H), 3.68 (s, 2H), 2.38-2.26 (m, 12H), 2.20 (br d, J=11.6 Hz, 1H), 2.06-2.02 (m, 1H), 1.18 (s, 3H).

and Peak 2, enantiomer 2, (R)—N-(1-(6-(1,1-difluoroethyl)pyrazin-2-yl)-3-(3-(dimethylamino)-3-methylpyrrolidin-1-yl)-1H-pyrazolo[4,3-c]pyridin-6-yl)acetamide or (S)—N-(1-(6-(1,1-difluoroethyl)pyrazin-2-yl)-3-(3-(dimethylamino)-3-methylpyrrolidin-1-yl)-1H-pyrazolo[4,3-c]pyridin-6-yl)acetamide (33 mg, 27.5% yield) as a yellow solid. LCMS m/z=445.3 [M+H]⁺. ¹H NMR (400 MHz, CDCl₃) δ: ppm 9.33 (s, 1H), 9.29 (s, 1H), 8.73 (s, 1H), 8.66 (s, 1H), 8.09 (s, 1H), 3.95-3.93 (m, 1H), 3.85-3.82 (m, 1H), 3.68 (s, 2H), 2.37-2.26 (m, 12H), 2.22-2.15 (m, 1H), 2.06-2.00 (m, 1H), 1.18 (s, 3H).

### Examples 302 and 303: (R)—N-(3-(3-(diethylamino)pyrrolidin-1-yl)-1-(6-(1,1-difluoroethyl)pyrazin-2-yl)-1H-pyrazolo[4,3-c]pyridin-6-yl)acetamide and (S)—N-(3-(3-(diethylamino)pyrrolidin-1-yl)-1-(6-(1,1-difluoroethyl)pyrazin-2-yl)-1H-pyrazolo[4,3-c]pyridin-6-yl)acetamide

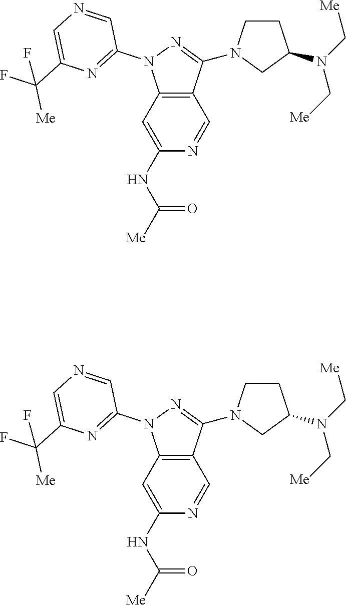 [SMILES: CCN(CC)[C@@H]1CCN(c2nn(-c3cncc(C(C)(F)F)n3)c3cc(NC(C)=O)ncc23)C1 ; CCN(CC)[C@H]1CCN(c2nn(-c3cncc(C(C)(F)F)n3)c3cc(NC(C)=O)ncc23)C1]

To a solution of N-(3-bromo-1-(6-(1,1-difluoroethyl)pyrazin-2-yl)-1H-pyrazolo[4,3-c]pyridin-6-yl)acetamide (Preparation 504, 150.0 mg, 0.378 mmol) in DMSO (5.0 mL) was added N,N-diethylpyrrolidin-3-amine (80.6 mg, 0.567 mmol), CuI (7.2 mg, 37.8 μmol), K₃PO₄ (240.5 mg, 1.13 mmol) and 2,6-DFPAO (15.2 mg, 75.5 μmol) and the reaction mixture was stirred at 130° C. for 2 h under microwave irradiation. The mixture was concentrated and purified by prep-HPLC-C (43% to 73% MeCN) to give N-(3-(3-(diethylamino)pyrrolidin-1-yl)-1-(6-(1,1-difluoroethyl)pyrazin-2-yl)-1H-pyrazolo[4,3-c]pyridin-6-yl)acetamide (49.0 mg, 28.3% yield) as a yellow solid. This was further separated by SFC Column: DAICEL CHIRALPAK IG (250 mm*30 mm,10 μm); (Condition: CO₂-EtOH (0.1% NH₃·H₂O); 45% isocratic gradient at 80 mL/min to give Peak 1, enantiomer 1, (R)—N-(3-(3-(diethylamino)pyrrolidin-1-yl)-1-(6-(1,1-difluoroethyl)pyrazin-2-yl)-1H-pyrazolo[4,3-c]pyridin-6-yl)acetamide or (S)—N-(3-(3-(diethylamino)pyrrolidin-1-yl)-1-(6-(1,1-difluoroethyl)pyrazin-2-yl)-1H-pyrazolo[4,3-c]pyridin-6-yl)acetamide (24 mg, 48.6% yield) as a white solid. LCMS m/z=459.3 [M+H]^{+ 1}H NMR (400 MHz, CDCl₃) δ ppm 9.32-9.28 (m, 2H), 8.74 (s, 1H), 8.67 (s, 1H), 8.10 (s, 1H), 4.02-3.92 (m, 2H), 3.76-3.55 (m, 3H), 2.83-2.80 (m, 4H), 2.36-2.26 (m, 8H), 1.15 (t, J=7.2 Hz, 6H).

and Peak 2, enantiomer 2, (S)—N-(3-(3-(diethylamino)pyrrolidin-1-yl)-1-(6-(1,1-difluoroethyl)pyrazin-2-yl)-1H-pyrazolo[4,3-c]pyridin-6-yl)acetamide or (R)—N-(3-(3-(diethylamino)pyrrolidin-1-yl)-1-(6-(1,1-difluoroethyl)pyrazin-2-yl)-1H-pyrazolo[4,3-c]pyridin-6-yl)acetamide (23.2 mg, 47.9% yield) as a white solid. LCMS m/z=459.3 [M+H]⁺ 1H NMR: (400 MHz, CDCl₃) δ ppm 9.32-9.27 (m, 2H), 8.73 (s, 1H), 8.66 (s, 1H), 8.12 (s, 1H), 4.01-3.94 (m, 2H), 3.74-3.62 (m, 3H), 2.83-2.77 (m, 4H), 2.36-2.14 (m, 8H), 1.14 (t, J=7.2 Hz, 6H).

### Examples 304 and 305: (S)—N-(1-(6-(1,1-difluoroethyl)pyrazin-2-yl)-3-(3-(isopropylamino)pyrrolidin-1-yl)-1H-pyrazolo[4,3-c]pyridin-6-yl)acetamide and (R)—N-(1-(6-(1,1-difluoroethyl)pyrazin-2-yl)-3-(3-(isopropylamino)pyrrolidin-1-yl)-1H-pyrazolo[4,3-c]pyridin-6-yl)acetamide

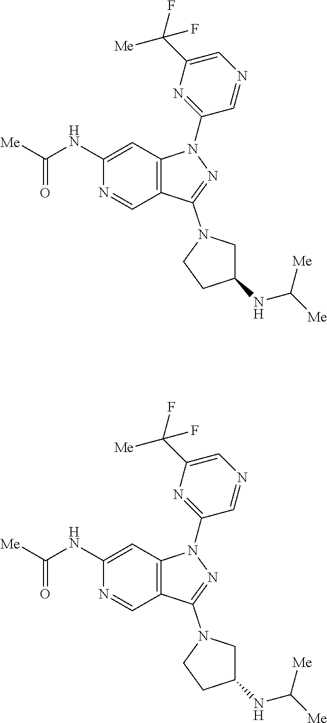 [SMILES: CC(=O)Nc1cc2c(cn1)c(N1CC[C@H](NC(C)C)C1)nn2-c1cncc(C(C)(F)F)n1 ; CC(=O)Nc1cc2c(cn1)c(N1CC[C@@H](NC(C)C)C1)nn2-c1cncc(C(C)(F)F)n1]

To a solution of N-(3-bromo-1-(6-(1,1-difluoroethyl)pyrazin-2-yl)-1H-pyrazolo[4,3-c]pyridin-6-yl)acetamide (Preparation 504, 150 mg, 0.378 mmol) in DMSO (8 mL) was added 3-(isopropylamino)pyrrolidine hydrochloride (96.8 mg, 0.588 mmol), CuI (14.4 mg, 75.6 μmol), K₃PO₄ (240.5 mg, 1.1 mmol), 2,6-DFPAO (30.4 mg, 0.151 mmol) and the reaction mixture was stirred at 100° C. for 12 h under N₂. The reaction was slowly quenched with H₂O (20 mL), the layers separated and the aqueous layer extracted with EtOAc (15 mL×3). The combined organic layers were washed with brine (15 mL), dried over Na₂SO₄, filtered and concentrated. The residue was purified with prep-HPLC-C (38% to 66% MeCN) to give N-(1-(6-(1,1-difluoroethyl)pyrazin-2-yl)-3-(3-(isopropylamino)pyrrolidin-1-yl)-1H-pyrazolo[4,3-c]pyridin-6-yl)acetamide (60 mg, 34.0% yield) as a white solid. This was separated by SFC Column: Phenomenex-Cellulose-2 (250 mm*30 mm, 10 μm), Condition: 0.1% NH₃·H₂O EtOH, 40% isocratic gradient at 80 mL/min to give Peak 1, enantiomer 1, (S)—N-(1-(6-(1,1-difluoroethyl)pyrazin-2-yl)-3-(3-(isopropylamino)pyrrolidin-1-yl)-1H-pyrazolo[4,3-c]pyridin-6-yl)acetamide or (R)—N-(1-(6-(1,1-difluoroethyl)pyrazin-2-yl)-3-(3-(isopropylamino)pyrrolidin-1-yl)-1H-pyrazolo[4,3-c]pyridin-6-yl)acetamide (16.8 mg, 21.1% yield) as a white solid. LCMS m/z=445.2 [M+H]⁺. ¹H NMR: (400 MHz, CDCl₃) δ ppm 9.28-9.25 (m, 2H), 8.77-8.62 (m, 2H), 8.25 (br s, 1H), 4.00-3.86 (m, 2H), 3.78-3.68 (m, 2H), 3.49 (br s, 1H), 3.10-2.95 (m, 1H), 2.40-2.20 (m, 7H), 2.01-1.93 (m, 1H), 1.16 (br s, 6H)

and Peak 2, enantiomer 2, (R)—N-(1-(6-(1,1-difluoroethyl)pyrazin-2-yl)-3-(3-(isopropylamino)pyrrolidin-1-yl)-1H-pyrazolo[4,3-c]pyridin-6-yl)acetamide or (S)—N-(1-(6-(1,1-difluoroethyl)pyrazin-2-yl)-3-(3-(isopropylamino)pyrrolidin-1-yl)-1H-pyrazolo[4,3-c]pyridin-6-yl)acetamide (20.5 mg, 36.0% yield) as a white solid. LCMS m/z=445.2 [M+H]⁺. 1H NMR (400 MHz, CDCl₃) δ ppm 9.26-9.19 (m, 2H), 8.67 (s, 1H), 8.59 (s, 1H), 8.00 (br s, 1H), 3.96-3.82 (m, 2H), 3.71-3.62 (m, 2H), 3.47-3.45 (m, 1H), 3.04-2.89 (m, 1H), 2.29-2.18 (m, 7H), 1.96-1.94 (m, 1H), 1.12-1.10 (m, 6H)

### Examples 306 and 307: (S)—N-(3-(3-(dimethylamino)-3-methylpyrrolidin-1-yl)-1-(6-(2-fluoropropan-2-yl)pyrazin-2-yl)-1H-pyrazolo[4,3-c]pyridin-6-yl)acetamide and (R)—N-(3-(3-(dimethylamino)-3-methylpyrrolidin-1-yl)-1-(6-(2-fluoropropan-2-yl)pyrazin-2-yl)-1H-pyrazolo[4,3-c]pyridin-6-yl)acetamide

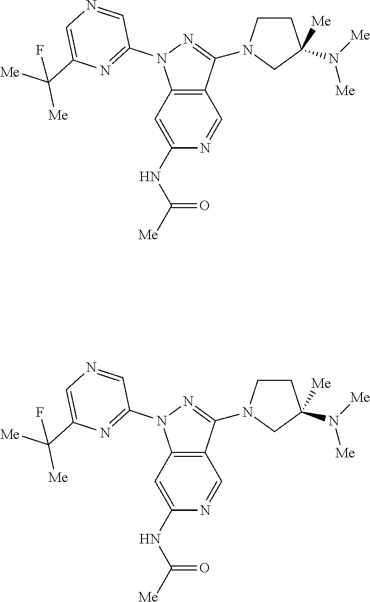 [SMILES: CC(=O)Nc1cc2c(cn1)c(N1CC[C@](C)(N(C)C)C1)nn2-c1cncc(C(C)(C)F)n1 ; CC(=O)Nc1cc2c(cn1)c(N1CC[C@@](C)(N(C)C)C1)nn2-c1cncc(C(C)(C)F)n1]

To a solution of N-(3-bromo-1-(6-(2-fluoropropan-2-yl)pyrazin-2-yl)-1H-pyrazolo[4,3-c]pyridin-6-yl)acetamide (Preparation 500, 250.0 mg, 0.636 mmol) in DMSO (5.0 mL) was added N,N,3-trimethylpyrrolidin-3-amine hydrochloride (293.2 mg, 1.78 mmol), K₃PO₄ (674.8 mg, 3.18 mmol), CuI (12.1 mg, 63.6 μmol) and 2,6-DFPAO (25.6 mg, 0.127 mmol) and the reaction mixture was stirred at 100° C. for 12 h under N₂. The mixture was concentrated and purified by prep-HPLC-C (38% to 68% MeCN) to give N-(3-(3-(dimethylamino)-3-methylpyrrolidin-1-yl)-1-(6-(2-fluoropropan-2-yl)pyrazin-2-yl)-1H-pyrazolo[4,3-c]pyridin-6-yl)acetamide (140 mg, 50% yield) as a yellow solid. This was further purified by SFC Column: CO₂-i-PrOH(0.10% NH₃·H₂O); 30% isocratic gradient at 80 mL/min to give Peak 1, enantiomer 1, (S)—N-(3-(3-(dimethylamino)-3-methylpyrrolidin-1-yl)-1-(6-(2-fluoropropan-2-yl)pyrazin-2-yl)-1H-pyrazolo[4,3-c]pyridin-6-yl)acetamide or (R)—N-(3-(3-(dimethylamino)-3-methylpyrrolidin-1-yl)-1-(6-(2-fluoropropan-2-yl)pyrazin-2-yl)-1H-pyrazolo[4,3-c]pyridin-6-yl)acetamide (49 mg, 37.7% yield) as a white solid. LCMS m/z=441.2 [M+H]⁺. ¹H NMR: (400 MHz, MeOD-d₄) δ ppm 9.32 (s, 1H), 9.04 (s, 1H), 8.87 (s, 1H), 8.49 (s, 1H), 3.99-3.68 (m, 4H), 2.49 (s, 6H), 2.49-2.16 (m, 5H), 1.91 (s, 3H), 1.86 (s, 3H), 1.31 (s, 3H).

and Peak 2, enantiomer 2, (R)—N-(3-(3-(dimethylamino)-3-methylpyrrolidin-1-yl)-1-(6-(2-fluoropropan-2-yl)pyrazin-2-yl)-1H-pyrazolo[4,3-c]pyridin-6-yl)acetamide or (S)—N-(3-(3-(dimethylamino)-3-methylpyrrolidin-1-yl)-1-(6-(2-fluoropropan-2-yl)pyrazin-2-yl)-1H-pyrazolo[4,3-c]pyridin-6-yl)acetamide (71 mg, 54.6% yield) as a white solid. LCMS m/z=441.2 [M+H]⁺ 1H NMR: (400 MHz, MeOD-d₄) δ ppm 9.29 (s, 1H), 9.01 (s, 1H), 8.85 (s, 1H), 8.48 (s, 1H), 3.97-3.69 (m, 4H), 2.53 (s, 6H), 2.25-2.14 (m, 5H), 1.90 (s, 3H), 1.84 (s, 3H), 1.32 (s, 3H).

### Examples 308 and 309: (S)—N-(1-(2-(2-fluoropropan-2-yl)-6-methylpyrimidin-4-yl)-3-(3′-methyl-[1,3′-bipyrrolidin]-1′-yl)-1H-pyrazolo[4,3-c]pyridin-6-yl)acetamide and (R)—N-(1-(2-(2-fluoropropan-2-yl)-6-methylpyrimidin-4-yl)-3-(3′-methyl-[1,3′-bipyrrolidin]-1′-yl)-1H-pyrazolo[4,3-c]pyridin-6-yl)acetamide

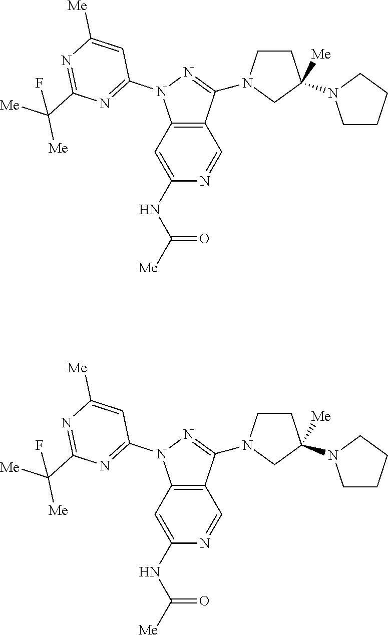 [SMILES: CC(=O)Nc1cc2c(cn1)c(N1CC[C@](C)(N3CCCC3)C1)nn2-c1cc(C)nc(C(C)(C)F)n1 ; CC(=O)Nc1cc2c(cn1)c(N1CC[C@@](C)(N3CCCC3)C1)nn2-c1cc(C)nc(C(C)(C)F)n1]

To a solution of N-(3-bromo-1-(2-(2-fluoropropan-2-yl)-6-methylpyrimidin-4-yl)-1H-pyrazolo[4,3-c]pyridin-6-yl)acetamide (Preparation 501, 150 mg, 0.37 mmol) in DMSO (3 mL) was added N,N,3-trimethylpyrrolidin-3-amine dihydrochloride (67.52 mg, 0.44 mmol), CuI (6.95 mg, 0.04 mmol), 2,6-DFPAO (7.34 mg, 0.04 mmol), K₃PO₄ (232.29 mg, 1.09 mmol) and the mixture was stirred in the microwave at 130° C. for 2 h under N₂. The mixture was concentrated and purified by Prep-HPLC-C (42% to 72% MeCN) to give N-(1-(2-(2-fluoropropan-2-yl)-6-methylpyrimidin-4-yl)-3-(3′-methyl-[1,3′-bipyrrolidin]-1′-yl)-1H-pyrazolo[4,3-c]pyridin-6-yl)acetamide (125 mg, 70.7% yield) as a white solid. This was further purified by SFC (Column: DAICEL CHIRALPAK IF (250 mm*30 mm,10 μm)); Mobile Phase: 30% of Heptane-EtOH (0.10% NH₃·H₂O) at 80 mL/min to give Peak 1, enantiomer 1, (S)—N-(1-(2-(2-fluoropropan-2-yl)-6-methylpyrimidin-4-yl)-3-(3′-methyl-[1,3′-bipyrrolidin]-1′-yl)-1H-pyrazolo[4,3-c]pyridin-6-yl)acetamide or (R)—N-(1-(2-(2-fluoropropan-2-yl)-6-methylpyrimidin-4-yl)-3-(3′-methyl-[1,3′-bipyrrolidin]-1′-yl)-1H-pyrazolo[4,3-c]pyridin-6-yl)acetamide (32 mg, 26.7%) as a white solid. LCMS m/z=444.2 [M+H]⁺. ¹H NMR (400 MHz, CDCl₃) δ: ppm 9.47 (s, 1H), 8.71 (s, 1H), 8.09 (s, 1H), 7.63 (s, 1H), 4.05-3.91 (m, 2H), 3.87-3.79 (m, 1H), 3.66 (d, J=9.6 Hz, 1H), 3.22-2.76 (m, 4H), 2.64 (s, 3H), 2.59-2.57 (m, 1H), 2.34-2.24 (m, 6H), 2.11-1.92 (m, 5H), 1.44 (s, 3H).

and Peak 2, enantiomer 2, (R)—N-(1-(2-(2-fluoropropan-2-yl)-6-methylpyrimidin-4-yl)-3-(3′-methyl-[1,3′-bipyrrolidin]-1′-yl)-1H-pyrazolo[4,3-c]pyridin-6-yl)acetamide or (S)—N-(1-(2-(2-fluoropropan-2-yl)-6-methylpyrimidin-4-yl)-3-(3′-methyl-[1,3′-bipyrrolidin]-1′-yl)-1H-pyrazolo[4,3-c]pyridin-6-yl)acetamide (24 mg, 20.0%) as a white solid. LCMS m/z=444.2 [M+H]⁺. ¹H NMR (400 MHz, CDCl₃) δ: ppm 9.47 (s, 1H), 8.71 (s, 1H), 8.09 (s, 1H), 7.63 (s, 1H), 4.05-3.95 (m, 2H), 3.87-3.81 (m, 1H), 3.68 (d, J=9.6 Hz, 1H), 3.22-2.76 (m, 4H), 2.67-2.65 (m, 4H), 2.33-2.23 (m, 6H), 2.13-1.96 (m, 5H), 1.44 (s, 3H).

### Examples 310 and 311: (R)—N-(3-(3-(dimethylamino)-3-methylpyrrolidin-1-yl)-1-(6-ethyl-2-(2-fluoropropan-2-yl)pyrimidin-4-yl)-1H-pyrazolo[4,3-c]pyridin-6-yl)acetamide and (S)—N-(3-(3-(dimethylamino)-3-methylpyrrolidin-1-yl)-1-(6-ethyl-2-(2-fluoropropan-2-yl)pyrimidin-4-yl)-1H-pyrazolo[4,3-c]pyridin-6-yl)acetamide

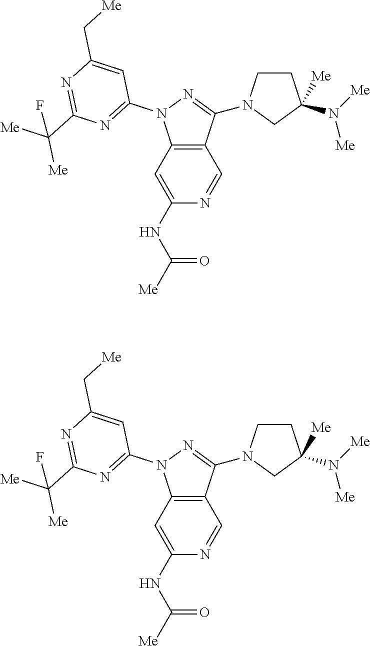 [SMILES: CCc1cc(-n2nc(N3CC[C@@](C)(N(C)C)C3)c3cnc(NC(C)=O)cc32)nc(C(C)(C)F)n1 ; CCc1cc(-n2nc(N3CC[C@](C)(N(C)C)C3)c3cnc(NC(C)=O)cc32)nc(C(C)(C)F)n1]

To a solution of N-(3-bromo-1-(6-ethyl-2-(2-fluoropropan-2-yl)pyrimidin-4-yl)-1H-pyrazolo[4,3-c]pyridin-6-yl)acetamide (Preparation 502, 200.0 mg, 0.475 mmol) and N,N,3-trimethylpyrrolidin-3-amine hydrochloride (234.5 mg, 1.42 mmol) in DMSO (3 mL) was added K₃PO₄ (302.3 mg, 1.42 mmol), CuI (18.1 mg, 0.095 mmol) and 2,6-DFPAO (19.1 mg, 0.095 mmol). The reaction was stirred at 130° C. for 2 h under microwave irradiation under N₂. The mixture was concentrated and purified by prep-HPLC-C (39% to 69% MeCN) to give N-(3-(3-(dimethylamino)-3-methylpyrrolidin-1-yl)-1-(6-ethyl-2-(2-fluoropropan-2-yl)pyrimidin-4-yl)-1H-pyrazolo[4,3-c]pyridin-6-yl)acetamide (145.2 mg, 65.3% yield) as a white solid. This was further separated by SFC (Column: DAICEL CHIRALPAK IC(250 mm*30 mm,10 um); Condition: 0.10% NH₃—H₂O MEOH; 55% isocratic gradient at 80 mL/min, to give Peak 1, enantiomer 1, (R)—N-(3-(3-(dimethylamino)-3-methylpyrrolidin-1-yl)-1-(6-ethyl-2-(2-fluoropropan-2-yl)pyrimidin-4-yl)-1H-pyrazolo[4,3-c]pyridin-6-yl)acetamide and (S)—N-(3-(3-(dimethylamino)-3-methylpyrrolidin-1-yl)-1-(6-ethyl-2-(2-fluoropropan-2-yl)pyrimidin-4-yl)-1H-pyrazolo[4,3-c]pyridin-6-yl)acetamide (58.7 mg, 40.4% yield) as a white solid. LCMS m/z=469.2 [M+H]^{+ 1}H NMR (500 MHz, CDCl₃) δ ppm: 9.45 (s, 1H), 8.71 (s, 1H), 8.15 (s, 1H), 7.52 (s, 1H), 3.94-3.92 (m, 1H), 3.83-3.81 (m, 1H), 3.69-3.66 (m, 2H), 2.88 (q, J=7.5 Hz, 2H), 2.36 (s, 6H), 2.26 (s, 3H), 2.18-2.16 (m, 1H), 2.02-2.01 (m, 1H), 1.98 (s, 3H), 1.93 (s, 3H), 1.37 (t, J=7.5 Hz, 3H), 1.16 (s, 3H).

and Peak 2, enantiomer 2, (S)—N-(3-(3-(dimethylamino)-3-methylpyrrolidin-1-yl)-1-(6-ethyl-2-(2-fluoropropan-2-yl)pyrimidin-4-yl)-1H-pyrazolo[4,3-c]pyridin-6-yl)acetamide or (R)—N-(3-(3-(dimethylamino)-3-methylpyrrolidin-1-yl)-1-(6-ethyl-2-(2-fluoropropan-2-yl)pyrimidin-4-yl)-1H-pyrazolo[4,3-c]pyridin-6-yl)acetamide (56.5 mg, 38.9% yield) as a white solid. LCMS m/z=469.2 [M+H]⁺. ¹H NMR (500 MHz, CDCl₃) δ ppm: 9.45 (s, 1H), 8.71 (s, 1H), 8.12 (s, 1H), 7.52 (s, 1H), 3.94-3.92 (m, 1H), 3.84-3.82 (m, 1H), 3.68-3.66 (m, 2H), 2.89 (q, J=7.5 Hz, 2H), 2.36 (s, 6H), 2.26 (s, 3H), 2.19-2.17 (m, 1H), 2.04-2.03 (m, 1H), 1.98 (s, 3H), 1.93 (s, 3H), 1.37 (t, J=7.5 Hz, 3H), 1.17 (s, 3H).

### Examples 312 and 313: (S)—N-(3-(3-(ethyl(methyl)amino)-3-methylpyrrolidin-1-yl)-1-(6-ethyl-2-(2-fluoropropan-2-yl)pyrimidin-4-yl)-1H-pyrazolo[4,3-c]pyridin-6-yl)acetamide and (R)—N-(3-(3-(ethyl(methyl)amino)-3-methylpyrrolidin-1-yl)-1-(6-ethyl-2-(2-fluoropropan-2-yl)pyrimidin-4-yl)-1H-pyrazolo[4,3-c]pyridin-6-yl)acetamide

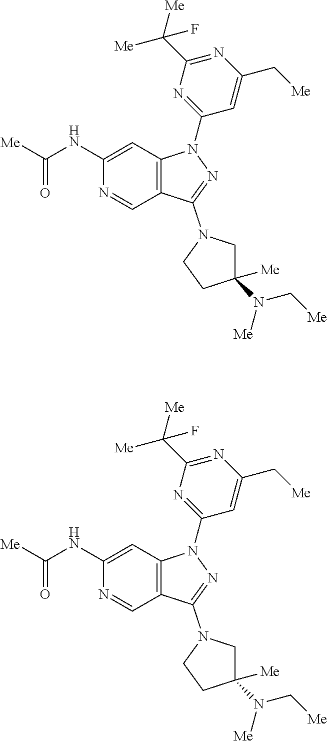 [SMILES: CCc1cc(-n2nc(N3CC[C@](C)(N(C)CC)C3)c3cnc(NC(C)=O)cc32)nc(C(C)(C)F)n1 ; CCc1cc(-n2nc(N3CC[C@@](C)(N(C)CC)C3)c3cnc(NC(C)=O)cc32)nc(C(C)(C)F)n1]

To a solution of N-(3-bromo-1-(6-ethyl-2-(2-fluoropropan-2-yl)pyrimidin-4-yl)-1H-pyrazolo[4,3-c]pyridin-6-yl)acetamide (Preparation 502, 200 mg, 0.47 mmol) in DMSO (5 mL) was added N-ethyl-N,3-dimethylpyrrolidin-3-amine (200.7 mg, 1.41 mmol), CuI (17.9 mg, 0.094 mmol), 2,6-DFPAO (28.4 mg, 0.141 mmol) and K₃PO₄ (299.5 mg, 1.41 mmol). The reaction was stirred at 130° C. for 2 h under N₂ under microwave irradiation. The mixture was purified by Prep-HPLC-P (50% to 80% MeCN) to give N-(3-(3-(ethyl(methyl)amino)-3-methylpyrrolidin-1-yl)-1-(6-ethyl-2-(2-fluoropropan-2-yl)pyrimidin-4-yl)-1H-pyrazolo[4,3-c]pyridin-6-yl)acetamide (120 mg, 52.4% yield) as a yellow solid. This was further separated by SFC (Column: DAICEL CHIRALPAK AD (250 mm*30 mm, 10 um); 50% isocratic gradient of 0.1% NH₃·H₂O EtOH at 80 mL/min; to give Peak 1, enantiomer 1, (S)—N-(3-(3-(ethyl(methyl)amino)-3-methylpyrrolidin-1-yl)-1-(6-ethyl-2-(2-fluoropropan-2-yl)pyrimidin-4-yl)-1H-pyrazolo[4,3-c]pyridin-6-yl)acetamide or (R)—N-(3-(3-(ethyl(methyl)amino)-3-methylpyrrolidin-1-yl)-1-(6-ethyl-2-(2-fluoropropan-2-yl)pyrimidin-4-yl)-1H-pyrazolo[4,3-c]pyridin-6-yl)acetamide (40 mg, 33.3% yield). LCMS m/z=487.3 [M+H]^{+ 1}H NMR: (400 MHz, CDCl₃) δ: ppm 9.48 (s, 1H), 8.71 (s, 1H), 8.07 (s, 1H), 7.63 (s, 1H), 3.96-3.91 (m, 1H), 3.82-3.80 (m, 1H), 3.73-3.68 (m, 2H), 2.93-2.88 (m, 2H), 2.51-2.49 (m, 2H), 2.33-2.23 (m, 10H), 2.08-2.06 (m, 1H), 1.40-1.36 (m, 3H), 1.18-1.14 (m, 6H).

and Peak 2, enantiomer 2, (R)—N-(3-(3-(ethyl(methyl)amino)-3-methylpyrrolidin-1-yl)-1-(6-ethyl-2-(2-fluoropropan-2-yl)pyrimidin-4-yl)-1H-pyrazolo[4,3-c]pyridin-6-yl)acetamide or (S)—N-(3-(3-(ethyl(methyl)amino)-3-methylpyrrolidin-1-yl)-1-(6-ethyl-2-(2-fluoropropan-2-yl)pyrimidin-4-yl)-1H-pyrazolo[4,3-c]pyridin-6-yl)acetamide (32 mg, 26.7% yield) as a white solid. LCMS m/z=487.3 [M+H]⁺. ¹H NMR: (400 MHz, CDCl₃) δ: ppm 9.48 (s, 1H), 8.71 (s, 1H), 8.07 (s, 1H), 7.63 (s, 1H), 3.94-3.92 (m, 1H), 3.85-3.80 (m, 1H), 3.74-3.68 (m, 2H), 2.93-2.88 (m, 2H), 2.52-2.48 (m, 2H), 2.30-2.24 (m, 10H), 2.08-2.03 (m, 1H), 1.40-1.36 (m, 3H), 1.19-1.17 (m, 6H).

### Examples 314 and 315: (R)—N-(1-(6-(1,1-difluoroethyl)pyridin-2-yl)-3-(3-(isopropylamino)pyrrolidin-1-yl)-1H-pyrazolo[4,3-c]pyridin-6-yl)acetamide and (S)—N-(1-(6-(1,1-difluoroethyl)pyridin-2-yl)-3-(3-(isopropylamino)pyrrolidin-1-yl)-1H-pyrazolo[4,3-c]pyridin-6-yl)acetamide

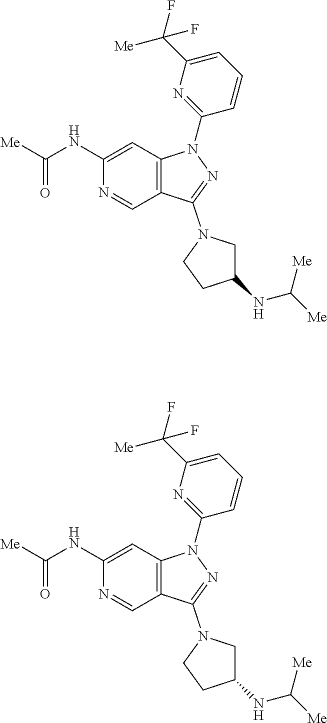

To a solution of N-(3-bromo-1-(6-(1,1-difluoroethyl)pyridin-2-yl)-1H-pyrazolo[4,3-c]pyridin-6-yl)acetamide (Preparation 510, 250 mg, 0.63 mmol) in DMSO (5 mL) was added N-isopropylpyrrolidin-3-amine hydrochloride (155.87 mg, 0.95 mmol), CuI (24.04 mg, 0.13 mmol), K₃PO₄ (401.83 mg, 1.89 mmol), 2,6-DFPAO (50.77 mg, 0.25 mmol) and the mixture was stirred at 130° C. for 2 hours under N₂. The mixture was concentrated and purified by Prep-HPLC-A (50% to 80% MeCN) to give N-(1-(6-(1,1-difluoroethyl)pyridin-2-yl)-3-(3-(isopropylamino)pyrrolidin-1-yl)-1H-pyrazolo[4,3-c]pyridin-6-yl)acetamide (130 mg, 46.5% yield) as a white solid.

This was further separated by SFC (Column: DAICEL CHIRALPAK AD (250 mm*30 mm, 10 um); Isocratic gradient: 35% [0.1% NH₃·H₂O EtOH] at 80 mL/min to give Peak 1, enantiomer 1, (R)—N-(1-(6-(1,1-difluoroethyl)pyridin-2-yl)-3-(3-(isopropylamino)pyrrolidin-1-yl)-1H-pyrazolo[4,3-c]pyridin-6-yl)acetamide or (S)—N-(1-(6-(1,1-difluoroethyl)pyridin-2-yl)-3-(3-(isopropylamino)pyrrolidin-1-yl)-1H-pyrazolo[4,3-c]pyridin-6-yl)acetamide (50 mg, 38.5%). LCMS m/z=444.2 [M+H]^{+ 1}H NMR: (500 MHz, CDCl₃) δ: ppm 9.36 (s, 1H), 8.70 (s, 1H), 8.12 (s, 1H), 7.95-7.82 (m, 2H), 7.41 (d, J=7.0 Hz, 1H), 4.00-3.98 (m, 1H), 3.87 (br s, 1H), 3.71-3.68 (m, 2H), 3.48 (br s, 1H), 3.01 (br s, 1H), 2.36-2.25 (m, 7H), 1.95 (br s, 1H), 1.16 (br s, 6H)

and Peak 2, enantiomer 2, (S)—N-(1-(6-(1,1-difluoroethyl)pyridin-2-yl)-3-(3-(isopropylamino)pyrrolidin-1-yl)-1H-pyrazolo[4,3-c]pyridin-6-yl)acetamide or (R)—N-(1-(6-(1,1-difluoroethyl)pyridin-2-yl)-3-(3-(isopropylamino)pyrrolidin-1-yl)-1H-pyrazolo[4,3-c]pyridin-6-yl)acetamide (50 mg, 38.5%) as a white solid. LCMS m/z=444.2 [M+H]^{+ 1}H NMR: (500 MHz, CDCl₃) δ: ppm 9.35 (s, 1H), 8.70 (s, 1H), 8.13 (s, 1H), 7.95-7.81 (m, 2H), 7.41 (d, J=7.5 Hz, 1H), 4.00-3.98 (m, 1H), 3.87 (br s, 1H), 3.71-3.69 (m, 2H), 3.49 (br s, 1H), 3.02 (br s, 1H), 2.35-2.25 (m, 7H), 1.96 (br s, 1H), 1.16 (br s, 6H)

### Example 316 and 317: (S)—N-(1-(6-(2-fluoropropan-2-yl)pyridin-2-yl)-3-(3-(isopropylamino)pyrrolidin-1-yl)-1H-pyrazolo[4,3-c]pyridin-6-yl)acetamide and (R)—N-(1-(6-(2-fluoropropan-2-yl)pyridin-2-yl)-3-(3-(isopropylamino)pyrrolidin-1-yl)-1H-pyrazolo[4,3-c]pyridin-6-yl)acetamide

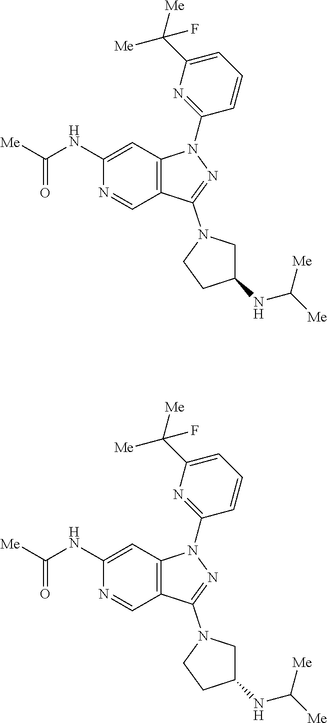 [SMILES: CC(=O)Nc1cc2c(cn1)c(N1CC[C@H](NC(C)C)C1)nn2-c1cccc(C(C)(C)F)n1 ; CC(=O)Nc1cc2c(cn1)c(N1CC[C@@H](NC(C)C)C1)nn2-c1cccc(C(C)(C)F)n1]

To a solution of N-(3-bromo-1-(6-(2-fluoropropan-2-yl)pyridin-2-yl)-1H-pyrazolo[4,3-c]pyridin-6-yl)acetamide (Preparation 511, 200 mg, 0.51 mmol) in DMSO (10 mL) was added N-isopropylpyrrolidin-3-amine hydrochloride (196.1 mg, 1.53 mmol), Cs₂CO₃ (332.3 mg, 1.02 mmol), 2,6-DFPAO (41.0 mg, 0.204 mmol) and CuI (19.4 mg, 0.102 mmol). The resulting mixture was stirred at 130° C. for 16 h under N₂. The mixture was concentrated and purified by Prep-HPLC-L (46% to 76% MeCN) to give N-(1-(6-(2-fluoropropan-2-yl)pyridin-2-yl)-3-(3-(isopropylamino)pyrrolidin-1-yl)-1H-pyrazolo[4,3-c]pyridin-6-yl)acetamide (117 mg, 52.2%) as a yellow solid. This was separated by SFC (Column:DAICEL CHIRALPAK AD (250 mm*30 mm,10 um); Mobile Phase: 35% [0.1% NH₃·H₂O EtOH]; at 100 mL/min to give Peak 1, enantiomer 1, (S)—N-(1-(6-(2-fluoropropan-2-yl)pyridin-2-yl)-3-(3-(isopropylamino)pyrrolidin-1-yl)-1H-pyrazolo[4,3-c]pyridin-6-yl)acetamide or (R)—N-(1-(6-(2-fluoropropan-2-yl)pyridin-2-yl)-3-(3-(isopropylamino)pyrrolidin-1-yl)-1H-pyrazolo[4,3-c]pyridin-6-yl)acetamide (35.1 mg, 34.4% yield) as yellow solid. LCMS m/z=440.3 [M+H]⁺. ¹H NMR (500 MHz, CDCl₃) δ: ppm 9.35 (s, 1H), 8.68 (s, 1H), 8.36 (br s, 1H), 7.81-7.71 (m, 2H), 7.31 (d, J=7.0 Hz, 1H), 3.99-3.85 (m, 2H), 3.75-3.66 (m, 2H), 3.57-3.41 (m, 1H), 3.07-2.97 (m, 1H), 2.36-2.31 (m, 1H), 2.25 (s, 3H), 1.98-1.95 (m, 1H), 1.93 (s, 3H), 1.88 (s, 3H), 1.17-1.14 (m, 6H).

and Peak 2, enantiomer 2, (R)—N-(1-(6-(2-fluoropropan-2-yl)pyridin-2-yl)-3-(3-(isopropylamino)pyrrolidin-1-yl)-1H-pyrazolo[4,3-c]pyridin-6-yl)acetamide or (S)—N-(1-(6-(2-fluoropropan-2-yl)pyridin-2-yl)-3-(3-(isopropylamino)pyrrolidin-1-yl)-1H-pyrazolo[4,3-c]pyridin-6-yl)acetamide (28.5 mg, 26.7%) as yellow solid. LCMS m/z=440.3 [M+H]^{+ 1}H NMR (500 MHz, CDCl₃) δ: ppm 9.35 (s, 1H), 8.68 (s, 1H), 8.36 (br s, 1H), 7.88-7.66 (m, 2H), 7.31 (d, J=7.0 Hz, 1H), 4.03-3.81 (m, 2H), 3.76-3.65 (m, 2H), 3.46-3.41 (m, 1H), 3.16-2.88 (m, 1H), 2.35-2.31 (m, 1H), 2.25 (s, 3H), 1.98-1.95 (m, 1H), 1.92 (s, 3H), 1.88 (s, 3H), 1.16 (t, J=6.0 Hz, 6H).

### Examples 318 and 319: (S)—N-(1-(2-(1,1-difluoroethyl)pyrimidin-4-yl)-3-(3′-methyl-[1,3′-bipyrrolidin]-1′-yl)-1H-pyrazolo[4,3-c]pyridin-6-yl)acetamide and (R)—N-(1-(2-(1,1-difluoroethyl)pyrimidin-4-yl)-3-(3′-methyl-[1,3′-bipyrrolidin]-1′-yl)-1H-pyrazolo[4,3-c]pyridin-6-yl)acetamide

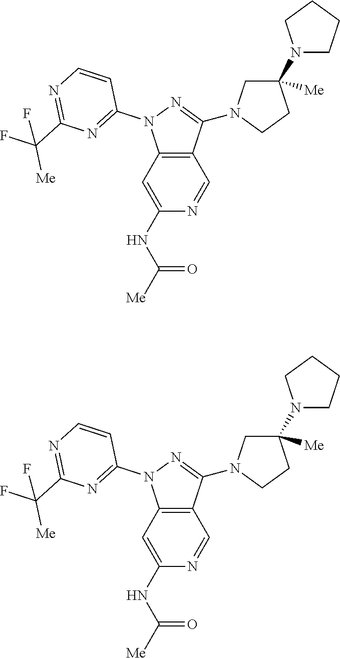 [SMILES: CC(=O)Nc1cc2c(cn1)c(N1CC[C@](C)(N3CCCC3)C1)nn2-c1ccnc(C(C)(F)F)n1 ; CC(=O)Nc1cc2c(cn1)c(N1CC[C@@](C)(N3CCCC3)C1)nn2-c1ccnc(C(C)(F)F)n1]

N-(1-(2-(1,1-Difluoroethyl)pyrimidin-4-yl)-3-(3′-methyl-[1,3′-bipyrrolidin]-1′-yl)-1H-pyrazolo[4,3-c]pyridin-6-yl)acetamide was obtained as a white solid, 150 mg, 63.3%, from N-(3-bromo-1-(2-(1,1-difluoroethyl)pyrimidin-4-yl)-1H-pyrazolo[4,3-c]pyridin-6-yl)acetamide (Preparation 368) and 3′-methyl-1,3′-bipyrrolidine, following a similar procedure to that described in Example 312. This was separated by SFC (Column: DAICEL CHIRALPAK AD (250 mm*30 mm, 10 um); Mobile Phase: 30% [0.1% NH₃·H₂O EtOH] at 80 mL/min to give Peak 1, enantiomer 1, (S)—N-(1-(2-(1,1-difluoroethyl)pyrimidin-4-yl)-3-(3′-methyl-[1,3′-bipyrrolidin]-1′-yl)-1H-pyrazolo[4,3-c]pyridin-6-yl)acetamide or (R)—N-(1-(2-(1,1-difluoroethyl)pyrimidin-4-yl)-3-(3′-methyl-[1,3′-bipyrrolidin]-1′-yl)-1H-pyrazolo[4,3-c]pyridin-6-yl)acetamide (27 mg, 18.0% yield) as a white solid. LCMS m/z=471.1 [M+H]^{+ 1}H NMR (400 MHz, DMSO-d₆) δ: ppm 10.75 (s, 1H), 9.37 (s, 1H), 8.95 (s, 1H), 8.83 (d, J=5.6 Hz, 1H), 7.78 (d, J=5.6 Hz, 1H), 3.89-3.74 (m, 2H), 3.69 (d, J=10.0 Hz, 1H), 3.56 (d, J=10.0 Hz, 1H), 2.68-2.62 (m, 4H), 2.26-2.17 (m, 4H), 2.15 (s, 3H), 1.94-1.86 (m, 1H), 1.69 (br s, 4H), 1.18 (s, 3H).

and Peak 2, enantiomer 2, (R)—N-(1-(2-(1,1-difluoroethyl)pyrimidin-4-yl)-3-(3′-methyl-[1,3′-bipyrrolidin]-1′-yl)-1H-pyrazolo[4,3-c]pyridin-6-yl)acetamide or (S)—N-(1-(2-(1,1-difluoroethyl)pyrimidin-4-yl)-3-(3′-methyl-[1,3′-bipyrrolidin]-1′-yl)-1H-pyrazolo[4,3-c]pyridin-6-yl)acetamide (21 mg, 14.0% yield) as white solid. LCMS m/z=471.1 [M+H]⁺. ¹H NMR (400 MHz, DMSO-d₆) δ: ppm 10.75 (s, 1H), 9.37 (s, 1H), 8.94 (s, 1H), 8.82 (d, J=5.6 Hz, 1H), 7.77 (d, J=5.6 Hz, 1H), 3.90-3.72 (m, 2H), 3.69 (d, J=10.0 Hz, 1H), 3.56 (d, J=10.0 Hz, 1H), 2.69-2.61 (m, 4H), 2.29-2.13 (m, 7H), 1.92-1.89 (m, 1H), 1.69 (br s, 4H), 1.17 (s, 3H).

### Example 320: N-(1-(2-(1,1-difluoroethyl)-6-methylpyrimidin-4-yl)-3-(2-methyl-2,6-diazaspiro[3.4]octan-6-yl)-1H-pyrazolo[4,3-c]pyridin-6-yl)acetamide

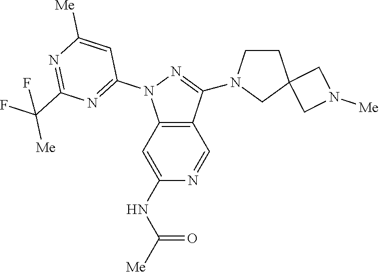 [SMILES: CC(=O)Nc1cc2c(cn1)c(N1CCC3(CN(C)C3)C1)nn2-c1cc(C)nc(C(C)(F)F)n1]

To a solution of N-(3-bromo-1-(2-(1,1-difluoroethyl)-6-methylpyrimidin-4-yl)-1H-pyrazolo[4,3-c]pyridin-6-yl)acetamide (Preparation 391, 70.0 mg, 0.17 mmol) in DMSO (2 mL) was added 2-methyl-2,6-diazaspiro[3.4]octane (27.9 mg, 0.22 mmol), CuI (6.5 mg, 0.034 mmol), L-Proline (5.9 mg, 0.051 mmol) and K₃PO₄ (72.3 mg, 0.341 mmol). The reaction was stirred at 100° C. for 16 h under N₂. The mixture was purified by prep-HPLC-C (35% to 65% MeCN) and the product further purified by prep-TLC (DCM/MeOH=10/1) to give N-(1-(2-(1,1-difluoroethyl)-6-methylpyrimidin-4-yl)-3-(2-methyl-2,6-diazaspiro[3.4]octan-6-yl)-1H-pyrazolo[4,3-c]pyridin-6-yl)acetamide (9.6 mg, 12.4% yield) as a white solid. LCMS m/z=457.1 [M+H]^{+ 1}H NMR: (400 MHz, CDCl₃) δ ppm: 9.45 (s, 1H), 8.72 (s, 1H), 8.13 (s, 1H), 7.61 (s, 1H), 3.93 (s, 2H), 3.80-3.76 (m, 2H), 3.69-3.57 (m, 3H), 2.64 (s, 3H), 2.60 (s, 3H), 2.41-2.38 (m, 2H), 2.33-2.23 (m, 7H).

Examples 321 to 335: The compounds in the following table were prepared from the appropriate bromide and amine, following a similar procedure to that described in Example 320.

Bromo pyrazolo[4,3-c]pyridine 8: N-(3-bromo-1-(2-(1,1-difluoroethyl)-6-methylpyrimidin-4-yl)-1H-pyrazolo[4,3-c]pyridin-6-yl)acetamide (Preparation 391); Bromo pyrazolo[4,3-c]pyridine 9: N-(3-bromo-1-(6-(1,1-difluoroethyl)pyridin-2-yl)-1H-pyrazolo[4,3-c]pyridin-6-yl)acetamide (Preparation 510); Bromo pyrazolo[4,3-c]pyridine 10: N-(3-bromo-1-(6-cyclopropyl-2-(1,1-difluoroethyl)pyrimidin-4-yl)-1H-pyrazolo[4,3-c]pyridin-6-yl)acetamide (Preparation 503); Bromo pyrazolo[4,3-c]pyridine 11: N-(3-bromo-1-(2-(1,1-difluoroethyl)-6-methoxypyrimidin-4-yl)-1H-pyrazolo[4,3-c]pyridin-6-yl)acetamide (Preparation 505)

| Example | |
|---|---|
| No | Name/Structure/Starting Materials (SM)/Data |
| 321 | N-(1-(2-(1,1-difluoroethyl)-6-methylpyrimidin-4-yl)-3-(5-methyl-2,5- diazaspiro[3.4]octan-2-yl)-1H-pyrazolo[4,3-c]pyridin-6-yl)acetamide 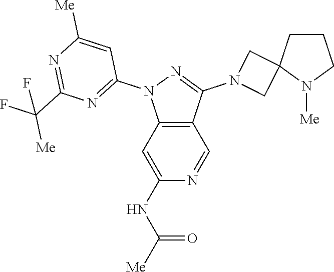 [SMILES: CC(=O)Nc1cc2c(cn1)c(N1CC3(CCCN3C)C1)nn2-c1cc(C)nc(C(C)(F)F)n1] SM: 5-methyl-2,5-diazaspiro[3.4]octane hydrochloride and Bromo pyrazolo[4,3-c]pyridine 8 prep-HPLC-C (33% to 60% MeCN). 28.9 mg, 26.0% yield as white solid. LCMS m/z = 457.1 [M + H]⁺ 1H NMR (500 MHz, CDCl₃) δ ppm: 9.45 (s, 1H), 8.55 (s, 1H), 8.07 (s, 1H), 7.63 (s, 1H), 4.44 (d, J = 8.0, 2H), 4.09 (d, J = 8.0, 2H), 2.81-2.77 (m, 2H), 2.64 (s, 3H), 2.56 (s, 3H), 2.33-2.32 (m, 1H), 2.28- 2.24 (m, 6H), 2.23-2.22 (m, 1H), 1.87-1.82 (m, 2H). |
| 322 | N-(1-(2-(1,1-difluoroethyl)-6-methylpyrimidin-4-yl)-3-(1-methyl-1,6- diazaspiro[3.3]heptan-6-yl)-1H-pyrazolo[4,3-c]pyridin-6-yl)acetamide 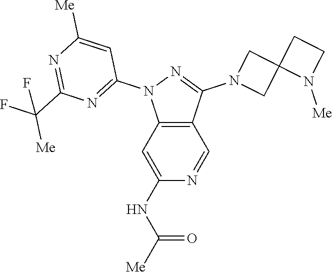 [SMILES: CC(=O)Nc1cc2c(cn1)c(N1CC3(CCN3C)C1)nn2-c1cc(C)nc(C(C)(F)F)n1] SM: 1-methyl-1,6-diazaspiro[3.3]heptane hydrochloride and Bromo pyrazolo[4,3-c]pyridine 8 prep-HPLC-C (41% to 71% MeCN). 18.2 mg, 33.8% yield as a white solid. LCMS m/z = 443.1 [M + H]⁺. 1H NMR (500 MHz, CDCl₃) δ ppm: 9.45 (s, 1H), 8.54 (s, 1H), 8.10 (s, 1H), 7.63 (s, 1H), 4.49 (d, J = 7.5 Hz, 2H), 4.27 (d, J = 8.5 Hz, 2H), 3.23-3.21 (m, 2H), 2.64 (s, 3H), 2.47-2.41 (m, 5H), 2.32-2.24 (m, 6H). |
| 323 | N-(1-(2-(1,1-difluoroethyl)-6-methylpyrimidin-4-yl)-3-(6-methyl-2,6- diazaspiro[3.3]heptan-2-yl)-1H-pyrazolo[4,3-c]pyridin-6-yl)acetamide 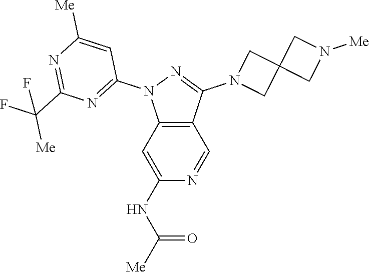 [SMILES: CC(=O)Nc1cc2c(cn1)c(N1CC3(CN(C)C3)C1)nn2-c1cc(C)nc(C(C)(F)F)n1] SM: 2-methyl-2,6-diazaspiro[3.3]heptane and Bromo pyrazolo[4,3-c]pyridine 8 prep-HPLC-F (42% to 72% MeCN) 12.0 mg, 22.3% yield as a white solid. LCMS m/z = 443.0 [M + H]⁺ 1H NMR: (500 MHz, CDCl₃) δ ppm: 9.43 (s, 1H), 8.51 (s, 1H), 8.05 (s, 1H), 7.62 (s, 1H), 4.39 (s, 4H), 3.48 (s, 4H), 2.65 (s, 3H), 2.36 (s, 3H), 2.32-2.23 (m, 6H). |
| 324 | N-(1-(2-(1,1-difluoroethyl)-6-methylpyrimidin-4-yl)-3-(3-methoxy-3- methylpyrrolidin-1-yl)-1H-pyrazolo[4,3-c]pyridin-6-yl)acetamide 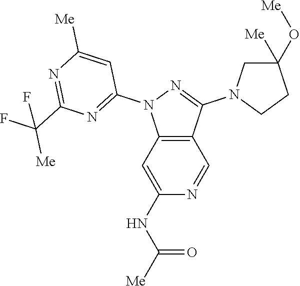 [SMILES: COC1(C)CCN(c2nn(-c3cc(C)nc(C(C)(F)F)n3)c3cc(NC(C)=O)ncc23)C1] SM: Bromo pyrazolo[4,3-c]pyridine 8 and 3-methoxy-3-methylpyrrolidine Prep-HPLC-P (37% to 67% MeCN) 35 mg, 30.2% yield as a yellow solid. LCMS m/z = 446.2 [M + H]⁺ ¹H NMR (400 MHz, CDCl₃) δ: ppm 9.58 (br s, 1H), 8.23 (s, 1H), 7.64 (s, 1H), 3.92-3.84 (m, 3H), 3.56 (d, J = 10.8 Hz, 1H), 3.31 (s, 3H), 2.64 (s, 3H), 2.37-2.24 (m, 7H), 2.03-1.97 (m, 1H), 1.49 (s, 3H). |
| 325 | N-(3-(6-oxa-3-azabicyclo[3.1.1]heptan-3-yl)-1-(2-(1,1-difluoroethyl)-6- methylpyrimidin-4-yl)-1H-pyrazolo[4,3-c]pyridin-6-yl)acetamide 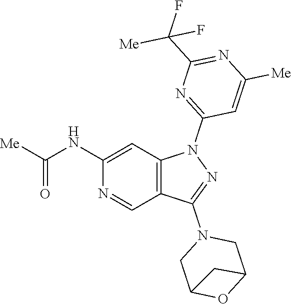 [SMILES: CC(=O)Nc1cc2c(cn1)c(N1CC3CC(C1)O3)nn2-c1cc(C)nc(C(C)(F)F)n1] SM: Bromo pyrazolo[4,3-c]pyridine 8 and 6-oxa-3-azabicyclo[3.1.1]heptane hydrochloride prep-HPLC-C (25% to 55% MeCN) 5.0 mg, 6.4% yield as a yellow solid. LCMS m/z = 430.1 [M + H]⁺. 1H NMR: (400 MHz, CDCl₃) δ ppm: 9.54 (s, 1H), 8.82 (s, 1H), 8.14 (br s, 1H), 7.67 (s, 1H), 4.84 (d, J = 6.4 Hz, 2H), 4.08- 4.11 (m, 2H), 3.98-4.01 (m, 2H), 3.36-3.43 (m, 1H), 2.65 (s, 3H), 2.25-2.34 (m, 6H), 2.13-2.17 (m, 1H). |
| 326 | N-(3-(3-cyclopropoxypyrrolidin-1-yl)-1-(2-(1,1-difluoroethyl)-6- methylpyrimidin-4-yl)-1H-pyrazolo[4,3-c]pyridin-6-yl)acetamide 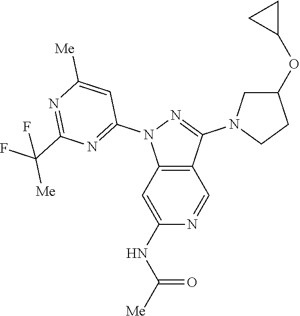 [SMILES: CC(=O)Nc1cc2c(cn1)c(N1CCC(OC3CC3)C1)nn2-c1cc(C)nc(C(C)(F)F)n1] SM: Bromo pyrazolo[4,3-c]pyridine 8 and 3-cyclopropoxypyrrolidine (Preparation 497) prep-HPLC-C (36% to 65% MeCN) 52.4 mg, 47.1% yield as a white solid. LCMS m/z = 458.1 [M + H]⁺ 1H NMR (400 MHz, CDCl₃) δ ppm: 9.55 (br s, 1H), 8.18 (br s, 1H), 7.63 (s, 1H), 4.41 (s, 1H), 3.90-3.83 (m, 4H), 3.41-3.37 (m, 1H), 2.63 (s, 3H), 2.33-2.18 (m, 8H), 0.64-0.54 (m, 4H). |
| 327 | N-(3-(2-oxa-5-azabicyclo[2.2.1]heptan-5-yl)-1-(6-(1,1-difluoroethyl)pyridin- 2-yl)-1H-pyrazolo[4,3-c]pyridin-6-yl)acetamide 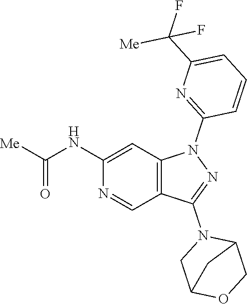 [SMILES: CC(=O)Nc1cc2c(cn1)c(N1CC3CC1CO3)nn2-c1cccc(C(C)(F)F)n1] SM: 2-oxa-5-azabicyclo[2.2.1]heptane and Bromo pyrazolo[4,3-c]pyridine 9 prep-HPLC-C (35% to 65% MeCN) 20.8 mg, 9.9% yield as a white solid. LCMS m/z = 415.1 [M + H]⁺1H NMR (400 MHz, MeOD-d₄) δ ppm: 9.34 (s, 1H), 8.80 (s, 1H), 8.00-7.96 (m, 2H), 7.47-7.45 (m, 1H), 4.85-4.83 (m, 1H), 4.76-4.75 (m, 1H), 4.09-4.07 (m, 1H), 3.93-3.89 (m, 2H), 3.69-3.66 (m, 1H), 2.29-2.23 (m, 6H), 2.19-2.04 (m, 2H). |
| 328 | N-(3-(6-oxa-3-azabicyclo[3.1.1]heptan-3-yl)-1-(6-(1,1-difluoroethyl)pyridin- 2-yl)-1H-pyrazolo[4,3-c]pyridin-6-yl)acetamide 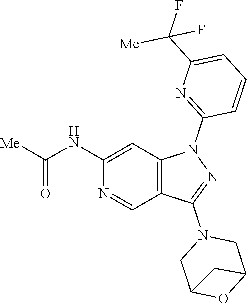 [SMILES: CC(=O)Nc1cc2c(cn1)c(N1CC3CC(C1)O3)nn2-c1cccc(C(C)(F)F)n1] SM: 6-oxa-3-azabicyclo[3.1.1]heptane and Bromo pyrazolo[4,3-c]pyridine 9 prep-HPLC-C (35% to 65% MeCN). 9.1 mg, 14.5% yield as a white solid. LCMS m/z = 415.1 [M + H]⁺ ¹H NMR (400 MHz, MeOD-d₄) δ ppm: 9.38 (s, 1H), 8.98 (s, 1H), 8.01 (s, 2H), 7.47 (s, 1H), 4.80-4.76 (m, 2H), 4.09-4.02 (m, 4H), 2.29-2.16 (m, 8H). |
| 329 | N-(1-(6-(1,1-difluoroethyl)pyridin-2-yl)-3-(3-(isopropylamino)pyrrolidin-1- yl)-1H-pyrazolo[4,3-c]pyridin-6-yl)acetamide 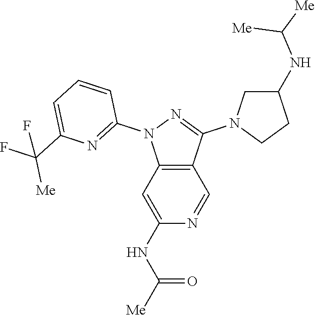 [SMILES: CC(=O)Nc1cc2c(cn1)c(N1CCC(NC(C)C)C1)nn2-c1cccc(C(C)(F)F)n1] SM: N-isopropylpyrrolidin-3-amine hydrochloride and Bromo pyrazolo[4,3- c]pyridine 9 Prep-HPLC-P (42% to 72% MeCN). 12 mg, 10.7 % yield as a yellow solid. LCMS m/z = 444.2 [M + H]⁺ ¹H NMR: (400 MHz, CDCl₃) δ: ppm 9.38 (s, 1H), 8.73 (s, 1H), 8.05 (s, 1H), 7.93 (d, J = 8.4 Hz, 1H), 7.88-7.84 (m, 1H), 7.42 (d, J = 7.2 Hz, 1H), 3.97-3.95 (m, 1H), 3.88-3.85 (m, 1H), 3.75-3.68 (m, 2H), 3.47- 3.45 (m, 1H), 3.01-2.98 (m, 1H), 2.37-2.25 (m, 7H), 1.92-1.91 (m, 1H), 1.15- 1.12 (m, 6H). |
| 330 | N-(1-(2-(1,1-difluoroethyl)-6-ethylpyrimidin-4-yl)-3-(3-(dimethylamino)-3- methylpyrrolidin-1-yl)-1H-pyrazolo[4,3-c]pyridin-6-yl)acetamide 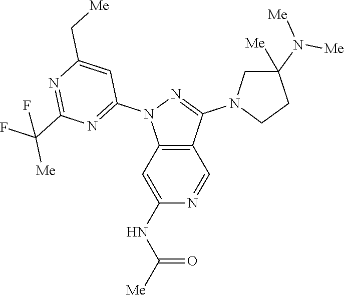 [SMILES: CCc1cc(-n2nc(N3CCC(C)(N(C)C)C3)c3cnc(NC(C)=O)cc32)nc(C(C)(F)F)n1] SM: N,N,3-trimethylpyrrolidin-3-amine and Bromo pyrazolo[4,3-c]pyridine 4 prep-HPLC-F (47% to 77% MeCN) 45.0 mg, 40.5% yield as a white solid. LCMS m/z = 473.2 [M + H]⁺ 1HNMR: (500 MHz, MeOD-d₄): δ ppm 9.40 (s, 1H), 8.89 (s, 1H), 7.73 (s, 1H), 4.02-3.90 (m, 1H), 3.90-3.88 (m, 1H), 3.81-3.79 (m, 1H), 3.68-3.66 (m, 1H), 2.91-2.86 (m, 2H), 2.45 (s, 6H), 2.27-2.23 (m, 6H), 2.19-2.15 (m, 2H), 1.37 (t, J = 7.5 Hz, 3H), 1.27 (s, 3H) |
| 331 | N-(1-(6-cyclopropyl-2-(1,1-difluoroethyl)pyrimidin-4-yl)-3-(3- (dimethylamino)-3-methylpyrrolidin-1-yl)-1H-pyrazolo[4,3-c]pyridin-6- yl)acetamide 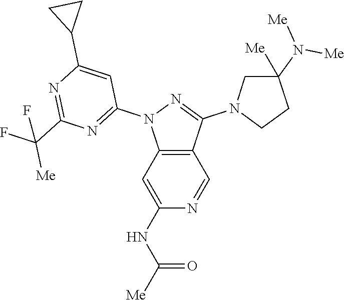 [SMILES: CC(=O)Nc1cc2c(cn1)c(N1CCC(C)(N(C)C)C1)nn2-c1cc(C2CC2)nc(C(C)(F)F)n1] SM: N,N,3-trimethylpyrrolidin-3-amine and Bromo pyrazolo[4,3-c]pyridine 10 prep-HPLC-C (45% to 75% MeCN) 54.6 mg, 39.4% yield as a white solid. LCMS m/z = 485.3 [M + H]⁺ ¹H NMR (400 MHz, CDCl₃) δ ppm: 9.49 (s, 1H), 8.71 (s, 1H), 8.04 (s, 1H), 7.59 (s, 1H), 3.97-3.92 (m, 1H), 3.84-3.82 (m, 1H), 3.70-3.67 (m, 2H), 2.37 (s, 6H), 2.26 (s, 3H), 2.21-2.12 (m, 5H), 2.03-2.02 (m, 1H), 1.26-1.24 (m, 2H), 1.17 (s, 3H), 1.11-1.09 (m, 2H). |
| 332 | N-(1-(2-(1,1-difluoroethyl)-6-methoxypyrimidin-4-yl)-3-(3-(dimethylamino)- 3-methylpyrrolidin-1-yl)-1H-pyrazolo[4,3-c]pyridin-6-yl)acetamide 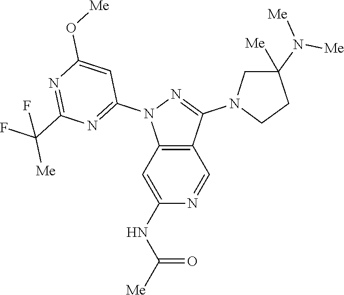 [SMILES: COc1cc(-n2nc(N3CCC(C)(N(C)C)C3)c3cnc(NC(C)=O)cc32)nc(C(C)(F)F)n1] SM: Bromo pyrazolo[4,3-c]pyridine 11 and N,N,3-trimethylpyrrolidin-3- amine prep-HPLC-C (40% to 70% MeCN) 12.1 mg, 15.6% yield as a white solid. LCMS m/z = 475.2 [M + H]⁺ 1H NMR: (400 MHz, CDCl₃) δ ppm: 9.43 (s, 1H), 8.68 (s, 1H), 8.21 (s, 1H), 7.11 (s, 1H), 4.07 (s, 3H), 3.91-3.88 (m, 1H), 3.82-3.77 (m, 1H), 3.64 (s, 2H), 2.35 (s, 6H), 2.30-2.20 (m, 6H), 2.19-2.12 (m, 1H), 2.01-1.98 (m, 1H), 1.16 (s, 3H). |
| 333^{A} | N-(3-(3-(diethylamino)-4-methylpyrrolidin-1-yl)-1-(2-(1,1-difluoroethyl)-6- ethylpyrimidin-4-yl)-1H-pyrazolo[4,3-c]pyridin-6-yl)acetamide 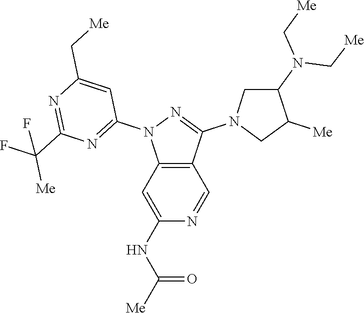 [SMILES: CCc1cc(-n2nc(N3CC(C)C(N(CC)CC)C3)c3cnc(NC(C)=O)cc32)nc(C(C)(F)F)n1] SM: N,N-diethyl-4-methylpyrrolidin-3-amine hydrochloride (Preparation 498) and Bromo pyrazolo[4,3-c]pyridine 4 Prep-HPLC-C (55% to 85% MeCN) 55 mg, yield: 17.8% as a white solid. LCMS m/z = 501.2 [M + H]⁺ 1H NMR (500 MHz, CDCl₃) δ ppm: 9.49 (s, 1H), 8.72 (s, 1H), 8.05 (s, 1H), 7.63 (s, 1H), 3.90-3.88 (m, 2H), 3.63-3.60 (m, 2H), 3.30 (br s, 1H), 2.91 (t, J = 7.5 Hz, 2H), 2.72 (br s, 4H), 2.55 (br s, 1H), 2.33- 2.23 (m, 6H), 1.38 (t, J = 7.5 Hz, 3H), 1.07 (br s, 9H) |
| 334 | N-(1-(2-(1,1-difluoroethyl)-6-ethylpyrimidin-4-yl)-3-(3-(dimethylamino)-3- ethylpyrrolidin-1-yl)-1H-pyrazolo[4,3-c]pyridin-6-yl)acetamide 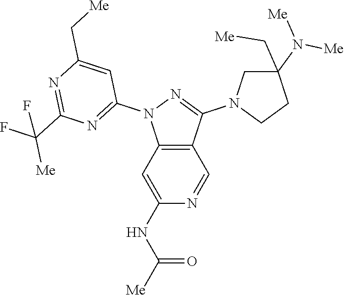 [SMILES: CCc1cc(-n2nc(N3CCC(CC)(N(C)C)C3)c3cnc(NC(C)=O)cc32)nc(C(C)(F)F)n1] SM: Bromo pyrazolo[4,3-c]pyridine 4 and 3-ethyl-N,N-dimethylpyrrolidin-3- amine Prep-HPLC-C (45% to 65% MeCN) 35 mg, 25.7% yield as a white solid. LCMS m/z = 487.3 [M + H]⁺ ¹H NMR (500 MHz, CDCl₃) δ ppm: 9.48 (s, 1H), 8.72 (s, 1H), 8.10 (s, 1H), 7.63 (s, 1H), 3.99- 3.78 (m, 3H), 3.63 (d, J = 10.2 Hz, 1H), 2.90 (q, J = 7.5 Hz, 2H), 2.43 (s, 7H), 2.33-2.24 (m, 6H), 2.10-2.02 (m, 1H), 1.85-1.77 (m, 2H), 1.38 (t, J = 7.5 Hz, 3H), 1.03 (t, J = 7.5 Hz, 3H) |
| 335 | N-(1-(2-(1,1-difluoroethyl)-6-ethylpyrimidin-4-yl)-3-(3- (isopropylamino)pyrrolidin-1-yl)-1H-pyrazolo[4,3-c]pyridin-6-yl)acetamide 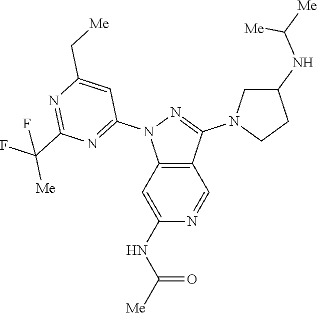 [SMILES: CCc1cc(-n2nc(N3CCC(NC(C)C)C3)c3cnc(NC(C)=O)cc32)nc(C(C)(F)F)n1] SM: Bromo pyrazolo[4,3-c]pyridine 4 and N-isopropylpyrrolidin-3-amine hydrochloride Prep-HPLC-P (34% to 64% MeCN) 45 mg, 40.5% yield as a yellow solid. LCMS m/z = 473.2 [M + H]⁺ ¹H NMR: (500 MHz, DMSO-d₆) δ: ppm 10.73 (s, 1H), 9.35 (s, 1H), 8.90 (s, 1H), 7.63 (s, 1H), 3.81-3.87 (m, 2H), 3.66-3.71 (m, 1H), 3.55 (brs, 1H), 3.37-3.38 (m, 1H), 3.30-3.32 (m, 1H), 2.83-2.86 (m, 3H), 2.50-2.52 (m, 1H), 2.18-2.26 (m, 3H), 2.15 (s, 3H), 1.77-1.90 (m, 1H), 1.28 (t, J = 7.5 Hz, 3H), 1.02 (t, J = 6.0 Hz, 6H). |

### A—Heated Under Microwave Irradiation

### Example 336: N-(3-(3-(bis(methyl-d₃)amino)pyrrolidin-1-yl)-1-(2-(1,1-difluoroethyl)-6-ethylpyrimidin-4-yl)-1H-pyrazolo[4,3-c]pyridin-6-yl)acetamide trifluoroacetate

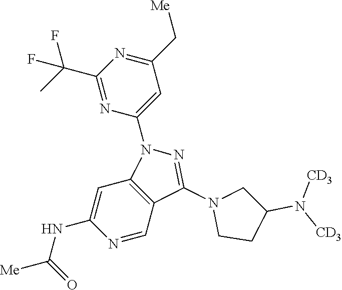 [SMILES: [2H]C([2H])([2H])N(C1CCN(c2nn(-c3cc(CC)nc(C(C)(F)F)n3)c3cc(NC(C)=O)ncc23)C1)C([2H])([2H])[2H]]

To a solution of N-(3-bromo-1-(2-(1,1-difluoroethyl)-6-ethylpyrimidin-4-yl)-1H-pyrazolo[4,3-c]pyridin-6-yl)acetamide (Preparation 393, 52 mg, 0.122 mmol) in DMSO (1.5 mL) was added N,N-bis(methyl-d₃)pyrrolidin-3-amine hydrochloride (Preparation 488, 44 mg, 0.227 mmol), CuI (2 mg, 12 μmol), K₃PO₄ (65 mg, 0.304 mmol) and L-Proline (3 mg, 24 μmol) and the reaction was sparged with N₂ for 5 minutes then stirred at 130° C. for 2 h under N₂ under microwave irradiation. Water was added and the mixture was extracted with EtOAc (2 mL×3). The combined organic layers were concentrated under vacuum and purified by Prep-HPLC-V (5% to 45% MeCN) to give N-(3-(3-(bis(methyl-d₃)amino)pyrrolidin-1-yl)-1-(2-(1,1-difluoroethyl)-6-ethylpyrimidin-4-yl)-1H-pyrazolo[4,3-c]pyridin-6-yl)acetamide TFA salt (6.1 mg, 8.7% yield) as yellow powder. LCMS m/z=465.4 [M+H]^{+ 1}H NMR (600 MHz, DMSO-d₆) δ ppm 10.74 (s, 1H), 10.05 (br s, 1H), 9.39 (s, 1H), 9.03 (s, 1H), 7.69 (s, 1H), 4.05-4.17 (m, 2H), 4.00 (m, 1H), 3.87 (m, 1H), 3.72-3.79 (m, 1H), 2.86 (m, 2H), 2.26-2.32 (m, 1H), 2.23 (t, J=19.3 Hz, 3H), 2.16 (s, 3H), 1.29 (t, J=7.6 Hz, 3H).

Examples 337 to 338: The Examples in the following table were prepared from the appropriate amine and N-(3-bromo-1-(2-(1,1-difluoroethyl)-6-ethylpyrimidin-4-yl)-1H-pyrazolo[4,3-c]pyridin-6-yl)acetamide (Preparation 393), following a similar procedure to that described in Example 336.

| Example | |
|---|---|
| No | Name, Structure, Starting amine (SM), Data |
| 337 | N-(3-(3-(bis(methyl-d₃)amino)-3-methylpyrrolidin-1-yl)-1-(2-(1,1- difluoroethyl)-6-ethylpyrimidin-4-yl)-1H-pyrazolo[4,3-c]pyridin-6- yl)acetamide 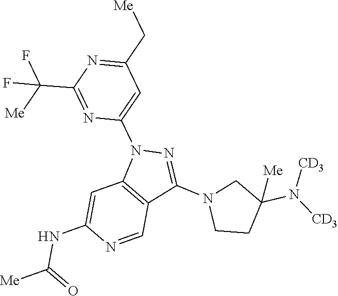 [SMILES: [2H]C([2H])([2H])N(C([2H])([2H])[2H])C1(C)CCN(c2nn(-c3cc(CC)nc(C(C)(F)F)n3)c3cc(NC(C)=O)ncc23)C1] SM: 3-methyl-N,N-bis(methyl-d₃)pyrrolidin-3-amine hydrochloride (Preparation 489) Prep-HPLC-W (Gradient: 10-90% MeCN). 14.7 mg, 25% yield as a white powder. LCMS m/z = 479.2 [M + H]⁺. ¹H NMR (600 MHz, DMSO-d₆) δ ppm 10.71 (s, 1H), 9.37 (s, 1H), 8.95 (d, J = 0.8 Hz, 1H), 7.66 (s, 1H), 3.90 (br t, J = 8.4 Hz, 1H), 3.75-3.84 (m, 1H), 3.71 (br d, J = 9.5 Hz, 1H), 3.54-3.62 (m, 1H), 2.85 (m, 2H), 2.17-2.27 (m, 3H), 2.15 (s, 3H), 1.96-2.10 (m, 2H), 1.28 (t, J = 7.6 Hz, 3H), 1.12 (br s, 3H). |
| 338 | N-(1-(2-(1,1-difluoroethyl)-6-ethylpyrimidin-4-yl)-3-(3-(methoxy-d₃)-3- methylpyrrolidin-1-yl)-1H-pyrazolo[4,3-c]pyridin-6-yl)acetamide trifluoroacetate 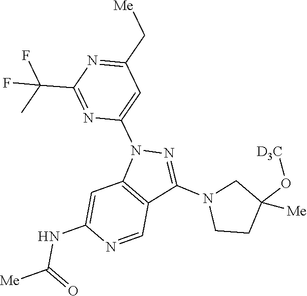 [SMILES: [2H]C([2H])([2H])OC1(C)CCN(c2nn(-c3cc(CC)nc(C(C)(F)F)n3)c3cc(NC(C)=O)ncc23)C1] SM: 3-(methoxy-d₃)-3-methylpyrrolidine hydrochloride (Preparation 490) Prep-HPLC-V (Gradient: 5-60% MeCN). 13.9 mg, 20% yield as a yellow solid. LCMS m/z = 463.4 [M + H]+. ¹H NMR (600 MHz, DMSO-d₆) δ ppm 10.73 (s, 1H), 9.36 (s, 1H), 8.94 (d, J = 0.8 Hz, 1H), 7.65 (s, 1H), 3.73-3.87 (m, 4H), 2.85 (m, 2H), 2.18-2.27 (m, 4H), 2.15 (s, 3H), 1.88-2.00 (m, 1H), 1.41 (s, 3H), 1.24-1.31 (m, 3H). |

### Example 339 and 340: (R)—N-(1-(2-(1,1-difluoroethyl)-6-methylpyrimidin-4-yl)-3-(3-ethoxypyrrolidin-1-yl)-1H-pyrazolo[4,3-c]pyridin-6-yl)acetamide and (S)—N-(1-(2-(1,1-difluoroethyl)-6-methylpyrimidin-4-yl)-3-(3-ethoxypyrrolidin-1-yl)-1H-pyrazolo[4,3-c]pyridin-6-yl)acetamide

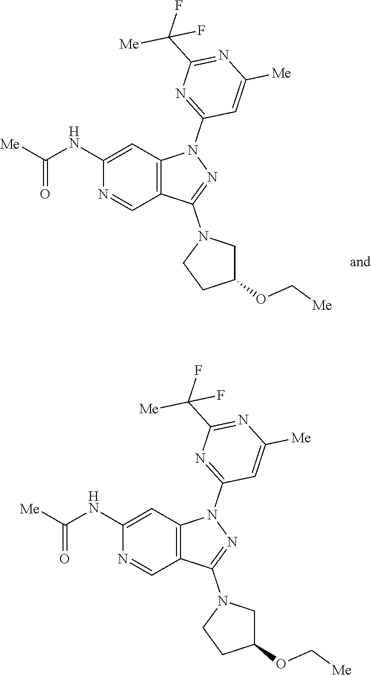 [SMILES: CCO[C@@H]1CCN(c2nn(-c3cc(C)nc(C(C)(F)F)n3)c3cc(NC(C)=O)ncc23)C1 ; CCO[C@H]1CCN(c2nn(-c3cc(C)nc(C(C)(F)F)n3)c3cc(NC(C)=O)ncc23)C1]

To a solution of N-(3-bromo-1-(2-(1,1-difluoroethyl)-6-methylpyrimidin-4-yl)-1H-pyrazolo[4,3-c]pyridin-6-yl)acetamide (Preparation 391, 300 mg, 0.73 mmol), CuI (13.9 mg, 0.073 mmol), L-proline (8.4 mg, 73.0 μmol) and K₃PO₄ (309.7 mg, 1.46 mmol) in DMSO (5.0 mL) was added 3-ethoxypyrrolidine hydrochloride (92.4 mg, 0.803 mmol). The mixture was stirred at 100° C. for 12 h under N₂. The mixture was purified by Prep-HPLC-Q (47% to 67% MeCN) give N-(1-(2-(1,1-difluoroethyl)-6-methylpyrimidin-4-yl)-3-(3-ethoxypyrrolidin-1-yl)-1H-pyrazolo[4,3-c]pyridin-6-yl)acetamide (110.0 mg, 33.9% yield) as a yellow solid. This was further purified by SFC (Column: DAICEL CHIRALPAK IG (250 mm*30 mm,10 um); Condition: 0.1% NH₃·H₂O EtOH; 40% isocratic gradient; at 80 ml/min) to give Peak 1, enantiomer 1, (R)—N-(1-(2-(1,1-difluoroethyl)-6-methylpyrimidin-4-yl)-3-(3-ethoxypyrrolidin-1-yl)-1H-pyrazolo[4,3-c]pyridin-6-yl)acetamide or (S)—N-(1-(2-(1,1-difluoroethyl)-6-methylpyrimidin-4-yl)-3-(3-ethoxypyrrolidin-1-yl)-1H-pyrazolo[4,3-c]pyridin-6-yl)acetamide (37.0 mg, 24.7% yield) as a white solid. LCMS m/z=446.1 [M+H]⁺ 1H NMR: (500 MHz, CDCl₃) δ ppm: 9.47 (s, 1H), 8.74 (s, 1H), 8.04 (s, 1H), 7.64 (s, 1H), 4.28 (s, 1H), 3.86-3.82 (m, 4H), 3.60-3.58 (m, 2H), 2.64 (s, 3H), 2.33-2.25 (m, 8H), 1.24 (t, J=7.0 Hz, 3H).

and Peak 2, enantiomer 2, (S)—N-(1-(2-(1,1-difluoroethyl)-6-methylpyrimidin-4-yl)-3-(3-ethoxypyrrolidin-1-yl)-1H-pyrazolo[4,3-c]pyridin-6-yl)acetamide or (R)—N-(1-(2-(1,1-difluoroethyl)-6-methylpyrimidin-4-yl)-3-(3-ethoxypyrrolidin-1-yl)-1H-pyrazolo[4,3-c]pyridin-6-yl)acetamide (37.0 mg, 24.7% yield) as a white solid. LCMS m/z=446.1 [M+H]⁺ 1H NMR: (500 MHz, CDCl₃) δ ppm: 9.52 (s, 1H), 8.72 (s, 1H), 7.65 (s, 1H), 4.29-4.28 (m, 1H), 3.89-3.81 (m, 4H), 3.60-3.58 (m, 2H), 2.64 (s, 3H), 2.33-2.19 (m, 8H), 1.25-1.23 (t, J=7.0 Hz, 3H).

### Example 341 and 342: N-(1-(2-(1,1-difluoroethyl)-6-methylpyrimidin-4-yl)-3-((3'S,4'S)-4′-methyl-[1,3′-bipyrrolidin]-1′-yl)-1H-pyrazolo[4,3-c]pyridin-6-yl)acetamide and N-(1-(2-(1,1-difluoroethyl)-6-methylpyrimidin-4-yl)-3-((3′R,4′R)-4′-methyl-[1,3′-bipyrrolidin]-1′-yl)-1H-pyrazolo[4,3-c]pyridin-6-yl)acetamide

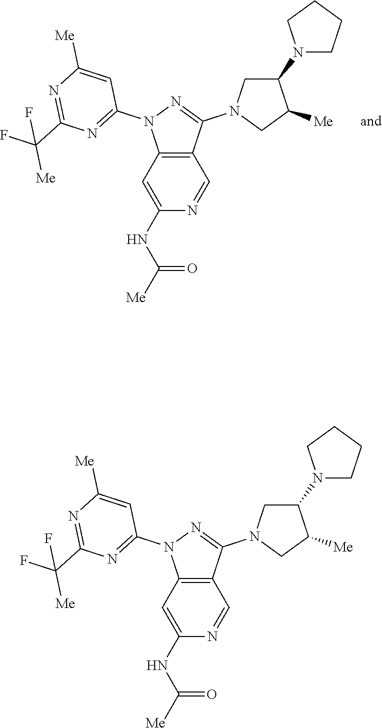 [SMILES: CC(=O)Nc1cc2c(cn1)c(N1C[C@@H](N3CCCC3)[C@@H](C)C1)nn2-c1cc(C)nc(C(C)(F)F)n1 ; CC(=O)Nc1cc2c(cn1)c(N1C[C@@H](C)[C@@H](N3CCCC3)C1)nn2-c1cc(C)nc(C(C)(F)F)n1]

N-(1-(2-(1,1-Difluoroethyl)-6-methylpyrimidin-4-yl)-3-(4′-methyl-[1,3′-bipyrrolidin]-1′-yl)-1H-pyrazolo[4,3-c]pyridin-6-yl)acetamide was obtained as a white solid, 100 mg, 84.9%, from N-(3-bromo-1-(2-(1,1-difluoroethyl)-6-methylpyrimidin-4-yl)-1H-pyrazolo[4,3-c]pyridin-6-yl)acetamide (Preparation 391) and 4′-methyl-1,3′-bipyrrolidine hydrochloride, following a similar procedure to that described in Example 339/340. The mixture was purified by prep-HPLC-C (45% to 75% MeCN) This was further purified by SFC (Column: DAICEL CHIRALPAK AD (250 mm*30 mm,10 um), Condition: CO₂-EtOH (0.1% NH₃H₂O); 40% isocratic gradient; Flow Rate (ml/min): 100) to give Peak 1, enantiomer 1, N-(1-(2-(1,1-difluoroethyl)-6-methylpyrimidin-4-yl)-3-((3'S,4'S)-4′-methyl-[1,3′-bipyrrolidin]-1′-yl)-1H-pyrazolo[4,3-c]pyridin-6-yl)acetamide or N-(1-(2-(1,1-difluoroethyl)-6-methylpyrimidin-4-yl)-3-((3′R,4′R)-4′-methyl-[1,3′-bipyrrolidin]-1′-yl)-1H-pyrazolo[4,3-c]pyridin-6-yl)acetamide (47.1 mg, 47.1% yield) as a white solid. LCMS m/z=485.3 [M+H]¹H NMR: (400 MHz, MeOD-d4) δ ppm: 9.37 (s, 1H), 8.86 (s, 1H), 7.71 (s, 1H), 3.96-3.88 (m, 2H), 3.70-3.67 (m, 2H), 3.12-2.53 (m, 9H), 2.28-2.18 (m, 6H), 2.01-1.88 (m, 4H), 1.17 (d, J=6.4 Hz, 3H).

and Peak 2, enantiomer 2, N-(1-(2-(1,1-difluoroethyl)-6-methylpyrimidin-4-yl)-3-((3′R,4′R)-4′-methyl-[1,3′-bipyrrolidin]-1′-yl)-1H-pyrazolo[4,3-c]pyridin-6-yl)acetamide or N-(1-(2-(1,1-difluoroethyl)-6-methylpyrimidin-4-yl)-3-((3'S,4'S)-4′-methyl-[1,3′-bipyrrolidin]-1′-yl)-1H-pyrazolo[4,3-c]pyridin-6-yl)acetamide (43.2 mg, 43.2% yield) as a white solid. LCMS m/z=485.3 [M+H]¹H NMR: (400 MHz, MeOD-d₄) δ ppm: 9.37 (s, 1H), 8.86 (s, 1H), 7.71 (s, 1H), 3.93-3.87 (m, 2H), 3.68-3.63 (m, 2H), 2.93-2.60 (m, 9H), 2.28-2.18 (m, 6H), 1.98-1.85 (m, 4H), 1.15 (d, J=7.2 Hz, 3H).

### Example 343: N-(3-(3-amino-3-methylpyrrolidin-1-yl)-1-(2-(1,1-difluoroethyl)-6-ethylpyrimidin-4-yl)-1H-pyrazolo[4,3-c]pyridin-6-yl)acetamide hydrochloride

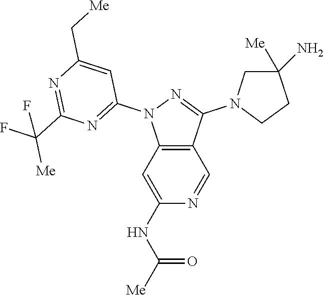 [SMILES: CCc1cc(-n2nc(N3CCC(C)(N)C3)c3cnc(NC(C)=O)cc32)nc(C(C)(F)F)n1]

Step 1: tert-Butyl (1-(6-acetamido-1-(2-(1,1-difluoroethyl)-6-ethylpyrimidin-4-yl)-1H-pyrazolo[4,3-c]pyridin-3-yl)-3-methylpyrrolidin-3-yl)carbamate was obtained as a yellow solid, 130 mg, 59.7% from N-(3-bromo-1-(2-(1,1-difluoroethyl)-6-ethylpyrimidin-4-yl)-1H-pyrazolo[4,3-c]pyridin-6-yl)acetamide (Preparation 393) and tert-butyl (3-methylpyrrolidin-3-yl)carbamate, following the procedure described in Preparation 511.

Step 2: To a solution of tert-butyl (1-(6-acetamido-1-(2-(1,1-difluoroethyl)-6-ethylpyrimidin-4-yl)-1H-pyrazolo[4,3-c]pyridin-3-yl)-3-methylpyrrolidin-3-yl)carbamate (130 mg, 0.24 mmol) in DCM (5 mL) was added HCl/dioxane (4 M, 1.5 mL) and the reaction stirred at 25° C. for 16 h. The mixture was concentrated and purified by prep-HPLC-I (10% to 30% MeCN) to give N-(3-(3-amino-3-methylpyrrolidin-1-yl)-1-(2-(1,1-difluoroethyl)-6-ethylpyrimidin-4-yl)-1H-pyrazolo[4,3-c]pyridin-6-yl)acetamide hydrochloride (37 mg, 34.9% yield) as a yellow solid. LCMS m/z=445.2 [M+H]⁺. ¹H NMR: (400 MHz, DMSO-d₆) δ ppm: 10.82 (s, 1H), 9.37 (s, 1H), 8.94 (s, 1H), 8.56 (br s, 3H), 7.66 (s, 1H), 4.05-4.03 (m, 1H), 3.97-3.91 (m, 2H), 3.71-3.69 (m, 1H), 2.89-2.83 (m, 2H), 2.33-2.15 (m, 8H), 1.55 (s, 3H), 1.28 (t, J=7.6 Hz, 3H).

Examples 344 to 347: The compounds in the following table were prepared from the appropriate protected pyrazolo[4,3-c]pyridine and halopyrimidine or halo pyridine, following a similar procedure to that described in Example 343.

| Example | |
|---|---|
| No | Name, Structure, Starting materials (SM), Data |
| 344 | N-(1-(2-(1,1-difluoroethyl)-6-ethylpyrimidin-4-yl)-3-(3-methyl-3- (methylamino)pyrrolidin-1-yl)-1H-pyrazolo[4,3-c]pyridin-6-yl)acetamide 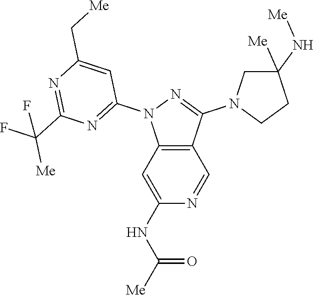 [SMILES: CCc1cc(-n2nc(N3CCC(C)(NC)C3)c3cnc(NC(C)=O)cc32)nc(C(C)(F)F)n1] SM: tert-butyl methyl(3-methylpyrrolidin-3-yl)carbamate and Bromo pyrazolo[4,3-c]pyridine 4 HPLC-C (35% to 65%). LCMS m/z = 459.4 [M + H]⁺. 1H NMR: (400 MHz, CDCl₃) δ ppm: 9.45 (s, 1H), 8.71 (s, 1H), 8.16 (s, 1H), 7.62 (s, 1H), 3.98- 3.94 (m, 1H), 3.81-3.76 (m, 2H), 3.59-3.56 (m, 1H), 2.93-2.87 (m, 2H), 2.50 (s, 3H), 2.32 (s, 1H), 2.28-2.26 (m, 4H), 2.23-2.21 (m, 1H), 2.03-1.99 (m, 2H), 1.42 (s, 3H), 1.39-1.36 (m, 3H). |
| 345 | N-(1-(2-(1,1-difluoroethyl)-6-ethylpyrimidin-4-yl)-3-(3-((1- methylcyclopropyl)amino)pyrrolidin-1-yl)-1H-pyrazolo[4,3-c]pyridin-6- yl)acetamide 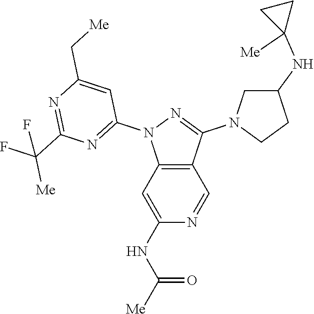 [SMILES: CCc1cc(-n2nc(N3CCC(NC4(C)CC4)C3)c3cnc(NC(C)=O)cc32)nc(C(C)(F)F)n1] SM: tert-butyl (1-methylcyclopropyl)(pyrrolidin-3-yl)carbamate (Preparation 484) and Bromo pyrazolo[4,3-c]pyridine 4 Prep-HPLC-F (55% to 85% MeCN). LCMS m/z = 485.2 [M + H]⁺ 1H NMR (500 MHz, MeOD-d₄) δ ppm 9.34 (br d, J = 7.5 Hz, 1H), 8.81 (br d, J = 8.5 Hz, 1H), 7.65 (br d, J = 8.5 Hz, 1H), 3.97-3.71 (m, 4H), 3.50-3.47 (m, 1H), 2.87 (q, J = 7.5 Hz, 2H), 2.34-2.32 (m, 1H), 2.27-2.18 (m, 6H), 2.06-1.95 (m, 1H), 1.37-1.34 (m, 6H), 0.66-0.49 (m, 2H), 0.50-0.46 (m, 2H). |
| 346 | N-(1-(6-(1,1-difluoroethyl)pyridin-2-yl)-3-(3-((1- methylcyclopropyl)amino)pyrrolidin-1-yl)-1H-pyrazolo[4,3-c]pyridin-6- yl)acetamide 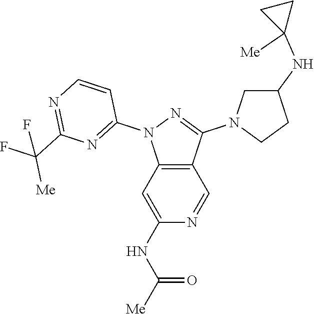 [SMILES: CC(=O)Nc1cc2c(cn1)c(N1CCC(NC3(C)CC3)C1)nn2-c1ccnc(C(C)(F)F)n1] SM: Bromo pyrazolo[4,3-c]pyridine 9 and tert-butyl (1- methylcyclopropyl)(pyrrolidin-3-yl)carbamate (Preparation 484), Prep-HPLC-C (45% to 71% MeCN). LCMS m/z = 456.2 [M + H]⁺. 1H NMR (400 MHz, MeOD-d₄) δ ppm 9.27 (s, 1H), 8.77 (s, 1H), 8.02-7.82 (m, 2H), 7.44-7.41 (m, 1H), 3.95-3.91 (m, 1H), 3.88-3.63 (m, 3H), 3.46-3.42 (m, 1H), 2.39-2.29 (m, 1H), 2.28-2.17 (m, 6H), 2.04-1.91 (m, 1H), 1.36 (s, 3H), 0.70- 0.59 (m, 2H), 0.51-0.41 (m, 2H). |
| 347 | (R)-N-(1-(6-(1,1-difluoroethyl)pyridin-2-yl)-3-(3-(methylamino)pyrrolidin- 1-yl)-1H-pyrazolo[4,3-c]pyridin-6-yl)acetamide 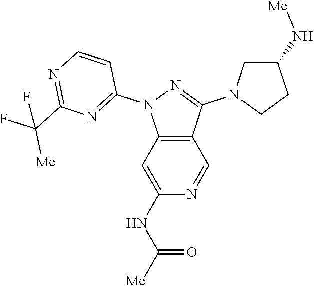 [SMILES: CN[C@@H]1CCN(c2nn(-c3ccnc(C(C)(F)F)n3)c3cc(NC(C)=O)ncc23)C1] SM: Bromo pyrazolo[4,3-c]pyridine 9 and tert-butyl (R)-methyl(pyrrolidin- 3-yl)carbamate hydrochloride Prep-HPLC-Q (10% to 40% MeCN). LCMS m/z = 416.3 [M + H]⁺ ¹HNMR (500 MHz, MeOD-d₄) δ ppm: 9.35 (s, 1H), 8.91 (m, 1H), 8.0-7.98 (m, 2H), 7.50-7.49 (m, 1H), 4.08-4.03 (m, 3H), 3.97-3.95 (m, 1H), 3.94-3.90 (m, 1H), 2.86 (s, 3H), 2.65-2.60 (m, 1H), 2.37-2.36 (m, 1H), 2.28-2.20 (m, 6H) |

### Example 348: N-(1-(2-(1,1-difluoroethyl)-6-methylpyrimidin-4-yl)-3-(3-isopropoxypyrrolidin-1-yl)-1H-pyrazolo[4,3-c]pyridin-6-yl)acetamide

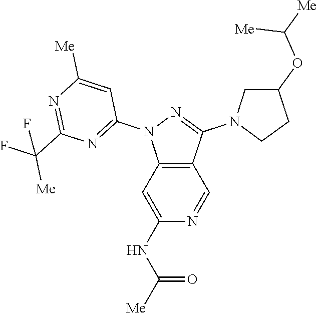 [SMILES: CC(=O)Nc1cc2c(cn1)c(N1CCC(OC(C)C)C1)nn2-c1cc(C)nc(C(C)(F)F)n1]

Zn (79.51 mg, 1.22 mmol) was stirred with 2N HCl for 4 min, then washed with water, EtOH, and PE, and dried under vacuum. To a solution of N-(3-bromo-1-(2-(1,1-difluoroethyl)-6-methylpyrimidin-4-yl)-1H-pyrazolo[4,3-c]pyridin-6-yl)acetamide (Preparation 391, 500 mg, 1.22 mmol) in DMPU (5 mL) was added 3-isopropoxypyrrolidine hydrochloride (235.7 mg, 1.82 mmol), Zn (79.51 mg, 1.22 mmol), DABCO (409.2 mg, 3.65 mmol), DBU (370.2 mg, 2.43 mmol) and NiBr·1,2-dimethoxyethane (3.75 mg, 12.2 mol) and the reaction mixture stirred at 55° C. for 12 h under N₂ atmosphere. The reaction mixture was diluted with H₂O (10 mL) and extracted with EtOAc (15 mL×3). The combined organic layers were dried over Na₂SO₄, filtered and concentrated under reduced pressure. The residue was purified by silica gel chromatography (ISCO®; 0 to 100% PE/EtOAc) to give N-(1-(2-(1,1-difluoroethyl)-6-methylpyrimidin-4-yl)-3-(3-isopropoxypyrrolidin-1-yl)-1H-pyrazolo[4,3-c]pyridin-6-yl)acetamide (36.6 mg, 6.5% yield) as a white solid. LCMS m/z=460.0 [M+H]⁺. 1H NMR (400 MHz, CDCl₃) δ ppm 9.51 (s, 1H), 8.73 (s, 1H), 8.60-8.38 (m, 1H), 7.65 (s, 1H), 4.42-4.36 (m, 1H), 3.92-3.85 (m, 2H), 3.84-3.79 (m, 1H), 3.78-3.70 (m, 2H), 2.65 (s, 3H), 2.33-2.24 (m, 6H), 2.22-2.17 (m, 2H), 1.24-1.19 (m, 6H).

### Example 349 and 350: (S)—N-(3-(3-(diethylamino)pyrrolidin-1-yl)-1-(2-(1,1-difluoroethyl)-6-isopropylpyrimidin-4-yl)-1H-pyrazolo[4,3-c]pyridin-6-yl)acetamide and (R)—N-(3-(3-(diethylamino)pyrrolidin-1-yl)-1-(2-(1,1-difluoroethyl)-6-isopropylpyrimidin-4-yl)-1H-pyrazolo[4,3-c]pyridin-6-yl)acetamide

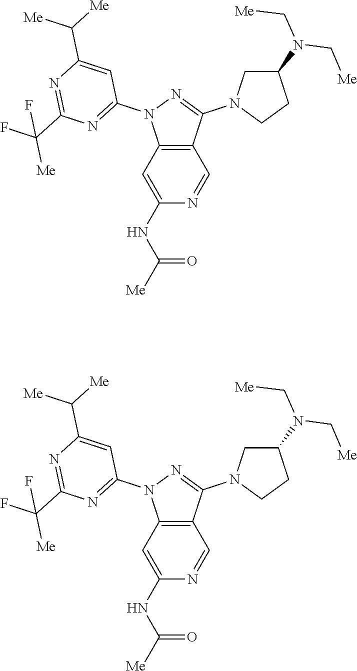 [SMILES: CCN(CC)[C@H]1CCN(c2nn(-c3cc(C(C)C)nc(C(C)(F)F)n3)c3cc(NC(C)=O)ncc23)C1 ; CCN(CC)[C@@H]1CCN(c2nn(-c3cc(C(C)C)nc(C(C)(F)F)n3)c3cc(NC(C)=O)ncc23)C1]

Zinc (59.6 mg, 0.911 mmol) was stirred with 2N HCl for 4 min, then washed with water, EtOH, and PE, and dried with high vacuum. To a solution of N-(3-bromo-1-(2-(1,1-difluoroethyl)-6-isopropylpyrimidin-4-yl)-1H-pyrazolo[4,3-c]pyridin-6-yl)acetamide (Preparation 508, 400 mg, 0.911 mmol) in DMPU (5.0 mL) was added N,N-diethylpyrrolidin-3-amine (194.3 mg, 1.37 mmol), Zinc (59.6 mg, 0.911 mmol), NiBr₂·1,2-dimethoxyethane (2.9 mg, 9.11 μmol), DABCO (306.5 mg, 2.73 mmol) and DBU (277.3 mg, 1.82 mmol) and the reaction mixture stirred at 55° C. for 16 h under N₂. The mixture was purified by prep-HPLC-F (63% to 93% MeCN) to give N-(3-(3-(diethylamino)pyrrolidin-1-yl)-1-(2-(1,1-difluoroethyl)-6-isopropylpyrimidin-4-yl)-1H-pyrazolo[4,3-c]pyridin-6-yl)acetamide (160 mg). This was further separated by SFC (Column: DAICEL CHIRALPAK IG (250 mm*30 mm,10 um); Condition: 0.1% NH₃·H₂O EtOH; 40% isocratic gradient, at 80 mL/min to give Peak 1, enantiomer 1, (S)—N-(3-(3-(diethylamino)pyrrolidin-1-yl)-1-(2-(1,1-difluoroethyl)-6-isopropylpyrimidin-4-yl)-1H-pyrazolo[4,3-c]pyridin-6-yl)acetamide or (R)—N-(3-(3-(diethylamino)pyrrolidin-1-yl)-1-(2-(1,1-difluoroethyl)-6-isopropylpyrimidin-4-yl)-1H-pyrazolo[4,3-c]pyridin-6-yl)acetamide (65.0 mg, 14.3% yield) as white solid. LCMS m/z=501.3 [M+H]⁺. ¹H NMR: (400 MHz, CDCl₃) δ ppm: 9.48 (s, 1H), 8.71 (s, 1H), 8.27 (s, 1H), 7.61 (s, 1H), 3.97-3.93 (m, 2H), 3.73-3.71 (m, 1H), 3.58-3.55 (m, 2H), 3.15-3.10 (m, 1H), 2.78-2.73 (m, 4H), 2.32-2.22 (m, 6H), 2.14-2.04 (m, 2H), 1.36 (d, J=6.8 Hz, 6H), 1.12 (t, J=7.2 Hz, 6);

and Peak 2, enantiomer 2, (R)—N-(3-(3-(diethylamino)pyrrolidin-1-yl)-1-(2-(1,1-difluoroethyl)-6-isopropylpyrimidin-4-yl)-1H-pyrazolo[4,3-c]pyridin-6-yl)acetamide or (S)—N-(3-(3-(diethylamino)pyrrolidin-1-yl)-1-(2-(1,1-difluoroethyl)-6-isopropylpyrimidin-4-yl)-1H-pyrazolo[4,3-c]pyridin-6-yl)acetamide (65.0 mg, 14.3% yield) as a white solid. LCMS m/z=501.3 [M+H]⁺. ¹H NMR: (400 MHz, CDCl₃) δ ppm: 9.47 (s, 1H), 8.70 (s, 1H), 8.23 (s, 1H), 7.61 (s, 1H), 3.98-3.93 (m, 2H), 3.73-3.71 (m, 1H), 3.63-3.61 (m, 2H), 3.15-3.10 (m, 1H), 2.83-2.76 (m, 4H), 2.31-2.22 (m, 6H), 2.18-2.11 (m, 2H), 1.37 (d, J=6.8 Hz, 6H), 1.14 (t, J=7.2 Hz, 6H).

### Example 351 and 352: (R)—N-(1-(2-(1,1-difluoroethyl)pyrimidin-4-yl)-3-(3-methoxy-3-methylpyrrolidin-1-yl)-1H-pyrazolo[4,3-c]pyridin-6-yl)acetamide and (S)—N-(1-(2-(1,1-difluoroethyl)pyrimidin-4-yl)-3-(3-methoxy-3-methylpyrrolidin-1-yl)-1H-pyrazolo[4,3-c]pyridin-6-yl)acetamide

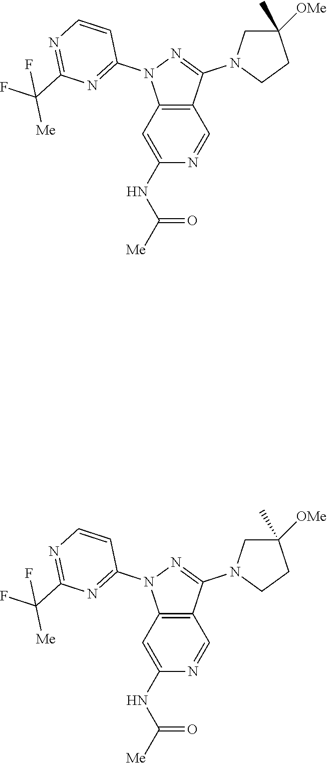 [SMILES: CO[C@]1(C)CCN(c2nn(-c3ccnc(C(C)(F)F)n3)c3cc(NC(C)=O)ncc23)C1 ; CO[C@@]1(C)CCN(c2nn(-c3ccnc(C(C)(F)F)n3)c3cc(NC(C)=O)ncc23)C1]

Zinc (32.93 mg, 0.504 mmol) was stirred with 2N HCl for 4 min, then washed with water, EtOH, and PE, and dried under vacuum. To a solution of N-(3-bromo-1-(2-(1,1-difluoroethyl)pyrimidin-4-yl)-1H-pyrazolo[4,3-c]pyridin-6-yl)acetamide (Preparation 368, 200 mg, 0.504 mmol) in DMPU (7 mL) was added 3-methoxy-3-methylpyrrolidine hydrochloride (152.7 mg, 1.01 mmol), Zinc (32.93 mg, 0.504 mmol), NiBr₂·1,2-dimethoxyethane (1.6 mg, 5.04 μmol), DABCO (169.5 mg, 1.51 mmol) and MTBD (154.3 mg, 1.01 mmol) and the reaction mixture was stirred at 55° C. for 16 h under N₂. The mixture was concentrated under vacuum and the residue purified by prep-HPLC-A (27% to 57% MeCN) to give N-(1-(2-(1,1-difluoroethyl)pyrimidin-4-yl)-3-(3-methoxy-3-methylpyrrolidin-1-yl)-1H-pyrazolo[4,3-c]pyridin-6-yl)acetamide (130 mg, 37.7% yield) as a white solid. This was further purified by SFC (Column: DAICEL CHIRALPAK AD (250 mm*30 mm,10 um); Condition: 40% [Neu-IPA] at 80 mL/min to give Peak 1, enantiomer 1, (R)—N-(1-(2-(1,1-difluoroethyl)pyrimidin-4-yl)-3-(3-methoxy-3-methylpyrrolidin-1-yl)-1H-pyrazolo[4,3-c]pyridin-6-yl)acetamide and (S)—N-(1-(2-(1,1-difluoroethyl)pyrimidin-4-yl)-3-(3-methoxy-3-methylpyrrolidin-1-yl)-1H-pyrazolo[4,3-c]pyridin-6-yl)acetamide (22 mg, 16.9% yield) as a white solid. LCMS m/z=432.1 [M+H]⁺. HNMR: (400 MHz, CDCl₃) δ ppm 9.52 (br s, 1H), 8.75-8.73 (m, 2H), 8.34 (br s, 1H), 7.78 (d, J=5.6 Hz, 1H), 3.91-3.83 (m, 3H), 3.54 (d, J=10.4 Hz, 1H), 3.31 (s, 3H), 2.33-2.24 (m, 7H), 2.02-1.97 (m, 1H), 1.49 (s, 3H).

and Peak 2, enantiomer 2, (S)—N-(1-(2-(1,1-difluoroethyl)pyrimidin-4-yl)-3-(3-methoxy-3-methylpyrrolidin-1-yl)-1H-pyrazolo[4,3-c]pyridin-6-yl)acetamide or (R)—N-(1-(2-(1,1-difluoroethyl)pyrimidin-4-yl)-3-(3-methoxy-3-methylpyrrolidin-1-yl)-1H-pyrazolo[4,3-c]pyridin-6-yl)acetamide (25 mg, 19.2% yield) as a white solid. LCMS m/z=432.1 [M+H]⁺. 1HNMR (400 MHz, CDCl₃) δ ppm: 9.45 (br s, 1H), 8.68-8.65 (m, 2H), 8.30 (br s, 1H), 7.72 (d, J=5.6 Hz, 1H), 3.85-3.76 (m, 3H), 3.47 (d, J=10.8 Hz, 1H), 3.24 (s, 3H), 2.27-2.17 (m, 7H), 1.96-1.90 (m, 1H), 1.42 (s, 3H).

### Example 353: N-(1-(2-(1,1-difluoroethyl)pyrimidin-4-yl)-3-(3-(difluoromethoxy)pyrrolidin-1-yl)-1H-pyrazolo[4,3-c]pyridin-6-yl)acetamide

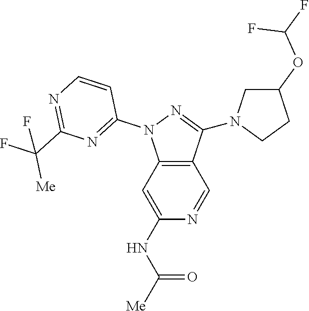 [SMILES: CC(=O)Nc1cc2c(cn1)c(N1CCC(OC(F)F)C1)nn2-c1ccnc(C(C)(F)F)n1]

To a solution of N-(3-bromo-1-(2-(1,1-difluoroethyl)pyrimidin-4-yl)-1H-pyrazolo[4,3-c]pyridin-6-yl)acetamide (Preparation 368, 100 mg, 0.252 mmol) in DMSO (3.0 mL) was added 3-(difluoromethoxy)pyrrolidine (172.6 mg, 1.26 mmol), NiBr₂;1,2-dimethoxyethane (3.9 mg, 12.6 μmol), Ru(bpy)₃(PF6)₂ (2.2 mg, 2.5 μmol) and DABCO (56.5 mg, 0.504 mmol). The mixture was irradiated (kessil 450 nM lamp) with heating (80° C.) for 16 h under N₂. The reaction mixture was purified by prep-HPLC-C (36% to 65% MeCN) to give N-(1-(2-(1,1-difluoroethyl)pyrimidin-4-yl)-3-(3-(difluoromethoxy)pyrrolidin-1-yl)-1H-pyrazolo[4,3-c]pyridin-6-yl)acetamide (18.7 mg, 15% yield) as a pink solid. LCMS m/z=454.1 [M+H]^{+ 1}H NMR: (500 MHz, CDCl₃) δ ppm: 9.51 (s, 1H), 8.75-8.73 (m, 2H), 8.04 (s, 1H), 7.79 (d, J=6.0 Hz, 1H), 6.31 (t, J=73.5 Hz, 1H), 5.09-5.07 (m, 1H), 3.97-3.89 (m, 4H), 2.38-2.34 (m, 2H), 2.29-2.25 (m, 6H).

Examples 354 to 357: The compounds in the following table were prepared from the appropriate Bromo pyrazolo[4,3-c]pyridine and amine, following a similar procedure to that described in Example 353.

| Example | |
|---|---|
| No | Name, Structure, Starting Materials, HPLC, Data |
| 354 | N-(1-(2-(1,1-difluoroethyl)pyrimidin-4-yl)-3-(3-methoxy-3-methylpyrrolidin- 1-yl)-1H-pyrazolo[4,3-c]pyridin-6-yl)acetamide 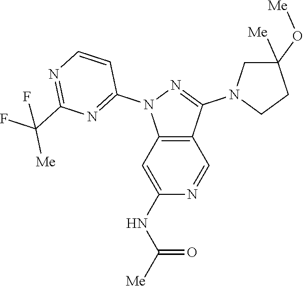 [SMILES: COC1(C)CCN(c2nn(-c3ccnc(C(C)(F)F)n3)c3cc(NC(C)=O)ncc23)C1] SM: 3-methoxy-3-methylpyrrolidine and Bromo pyrazolo[4,3-c]pyridine 3 prep-HPLC-C (35% to 64% MeCN) 21 mg, 19.3% yield as white solid. LCMS m/z = 432.1 [M + H]⁺. 1H NMR (400 MHz, CDCl₃) δ ppm: 9.48 (s, 1H), 8.72-8.73 (m, 2H), 8.07 (s, 1H), 7.78 (d, J = 5.6 Hz, 1H), 3.83-3.92 (m, 3H), 3.52-3.55 (m, 1H), 3.31 (s, 3H), 2.24- 2.34 (m, 7H), 1.94-2.00 (m, 1H), 1.49 (s, 3H). |
| 355 | N-(3-(2-oxa-5-azabicyclo[2.2.1]heptan-5-yl)-1-(2-(1,1-difluoroethyl)-6- methylpyrimidin-4-yl)-1H-pyrazolo[4,3-c]pyridin-6-yl)acetamide 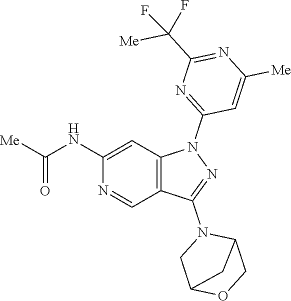 [SMILES: CC(=O)Nc1cc2c(cn1)c(N1CC3CC1CO3)nn2-c1cc(C)nc(C(C)(F)F)n1] SM: Bromo pyrazolo[4,3-c]pyridine 8 and 2-oxa-5-azabicyclo[2.2.1]heptane hydrochloride prep-HPLC-C (25% to 54% MeCN). 10.5 mg, 10.0% as white solid. LCMS m/z = 430.1 [M + H]⁺. 1H NMR (400 MHz, CDCl₃) δ ppm: 9.51 (s, 1H), 8.64 (s, 1H), 8.25 (s, 1H), 7.63 (s, 1H), 4.81-4.78 (m, 2H), 4.14-4.11 (m, 1H), 3.97-3.94 (m, 1H), 3.90-3.87 (m, 1H), 3.75-3.72 (m, 1H), 2.66 (s, 3H), 2.33- 2.23 (m, 6H), 2.12-2.07 (m, 2H). |
| 356 | N-(3-(2-oxa-5-azabicyclo[2.2.1]heptan-5-yl)-1-(2-(1,1-difluoroethyl)-6- methoxypyrimidin-4-yl)-1H-pyrazolo[4,3-c]pyridin-6-yl)acetamide 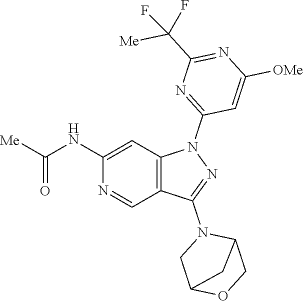 [SMILES: COc1cc(-n2nc(N3CC4CC3CO4)c3cnc(NC(C)=O)cc32)nc(C(C)(F)F)n1] SM: Bromo pyrazolo[4,3-c]pyridine 11 and 2-oxa-5-azabicyclo[2.2.1]heptane hydrochloride prep-HPLC-C (35% to 62% MeCN). 3.4 mg, 3.6% yield as a white solid. LCMS m/z = 446.1 [M + H]⁺ 1H NMR (400 MHz, CDCl₃) δ ppm: 9.46 (br s, 1H), 8.63 (s, 1H), 8.03 (br s, 1H), 7.10 (s, 1H), 4.78-4.75 (m, 2H), 4.13-4.11 (m, 1H), 4.08 (s, 3H), 3.94-3.86 (m, 2H), 3.73-3.70 (m, 1H), 2.30-2.20 (m, 6H), 2.07-2.02 (m, 2H). |
| 357 | N-(3-(6-oxa-3-azabicyclo[3.1.1]heptan-3-yl)-1-(2-(1,1-difluoroethyl)-6- methoxypyrimidin-4-yl)-1H-pyrazolo[4,3-c]pyridin-6-yl)acetamide 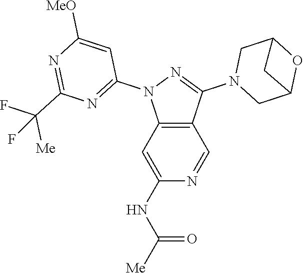 [SMILES: COc1cc(-n2nc(N3CC4CC(C3)O4)c3cnc(NC(C)=O)cc32)nc(C(C)(F)F)n1] SM: Bromo pyrazolo[4,3-c]pyridine 11 and 6-oxa-3-azabicyclo[3.1.1]heptane hydrochloride prep-HPLC-C (35% to 64% MeCN) 11.3 mg, 9.4% yield as a white solid. LCMS m/z = 446.1 [M + H]⁺. 1H NMR (400 MHz, CDCl₃) δ ppm: 9.51 (s, 1H), 8.79 (s, 1H), 8.17 (s, 1H), 7.14 (s, 1H), 4.83-4.82 (m, 2H), 4.08-3.95 (m, 7H), 4.40-3.35 (m, 1H), 2.31-2.13 (m, 7H). |

### Example 358: N-(3-(3-cyano-3-methylpyrrolidin-1-yl)-1-(2-(1,1-difluoroethyl)-6-methylpyrimidin-4-yl)-1H-pyrazolo[4,3-c]pyridin-6-yl)acetamide

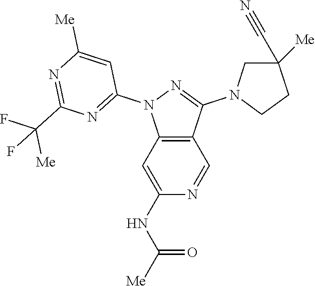 [SMILES: CC(=O)Nc1cc2c(cn1)c(N1CCC(C)(C#N)C1)nn2-c1cc(C)nc(C(C)(F)F)n1]

To a solution of N-(3-bromo-1-(2-(1,1-difluoroethyl)-6-methylpyrimidin-4-yl)-1H-pyrazolo[4,3-c]pyridin-6-yl)acetamide (Preparation 391, 100 mg, 0.243 mmol) in dioxane (8 mL) was added 3-methylpyrrolidine-3-carbonitrile (53.48 mg, 0.365 mmol), Xantphos (28.1 mg, 0.049 mmol), Cs₂CO₃ (237.7 mg, 0.73 mmol) and Pd₂(dba)₃ (22.3 mg, 0.024 mmol) and the reaction mixture was stirred at 100° C. for 12 h under N₂. The reaction mixture was concentrated under reduced pressure and the residue was purified by prep-HPLC-C (35% to 63% MeCN) to give N-(3-(3-cyano-3-methylpyrrolidin-1-yl)-1-(2-(1,1-difluoroethyl)-6-methylpyrimidin-4-yl)-1H-pyrazolo[4,3-c]pyridin-6-yl)acetamide (42.3 mg, 41.4% yield) as a white solid. LCMS m/z=441.1 [M+H]⁺. ¹H NMR (500 MHz, CDCl₃) δ ppm=9.49 (s, 1H), 8.70 (s, 1H), 8.18 (s, 1H), 7.62 (s, 1H), 4.21 (d, J=10.0 Hz, 1H), 4.09-4.03 (m, 1H), 4.00-3.96 (m, 1H), 3.66 (d, J=10.5 Hz, 1H), 2.65-2.60 (m, 4H), 2.32-2.24 (m, 6H), 2.22-2.15 (m, 1H), 1.66 (s, 3H).

Examples 359 to 362: The compounds in the following table were prepared from the appropriate bromo pyrazolo[4,3-c]pyridine and amine following a similar procedure to that described in Example 358.

| Example | |
|---|---|
| No | Name, Structure, Starting Materials (SM), HPLC, Data |
| 359 | N-(3-(3-cyano-3-ethylpyrrolidin-1-yl)-1-(2-(1,1-difluoroethyl)-6- methylpyrimidin-4-yl)-1H-pyrazolo[4,3-c]pyridin-6-yl)acetamide 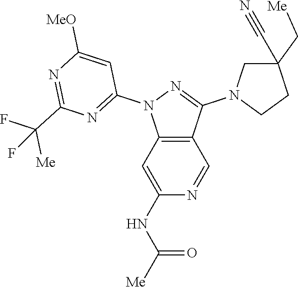 [SMILES: CCC1(C#N)CCN(c2nn(-c3cc(OC)nc(C(C)(F)F)n3)c3cc(NC(C)=O)ncc23)C1] SM: 3-ethylpyrrolidine-3-carbonitrile hydrochloride and Bromo pyrazolo[4,3- c]pyridine 8 prep-HPLC-C (36% to 66% MeCN). 43.0 mg, 38.9% yield as a white solid. LCMS m/z = 455.2 [M + H]⁺ 1H NMR (500 MHz, CDCl₃) δ ppm: 9.49 (s, 1H), 8.72 (s, 1H), 8.08 (s, 1H), 7.65 (s, 1H), 4.22-4.20 (m, 1H), 4.07-3.98 (m, 2H), 3.65-3.62 (m, 1H), 2.65-2.50 (m, 4H), 2.32-2.24 (m, 6H), 2.19-2.16 (m, 1H), 1.91-1.88 (m, 2H), 1.26 (t, J = 7.5 Hz, 3H). |
| 360 | N-(3-(3-cyano-3-methylpyrrolidin-1-yl)-1-(2-(1,1-difluoroethyl)-6- ethylpyrimidin-4-yl)-1H-pyrazolo[4,3-c]pyridin-6-yl)acetamide 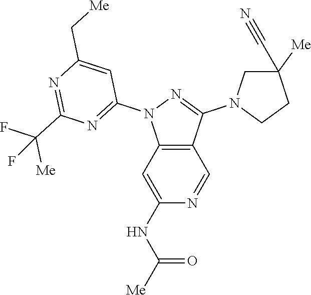 [SMILES: CCc1cc(-n2nc(N3CCC(C)(C#N)C3)c3cnc(NC(C)=O)cc32)nc(C(C)(F)F)n1] SM: 3-methylpyrrolidine-3-carbonitrile and Bromo pyrazolo[4,3-c]pyridine 4 prep-HPLC-C (38% to 68%). 47 mg, 44.0% yield as a white solid. LCMS m/z = 455.2 [M + H]⁺. 1H NMR: (500 MHz, CDCl₃) δ ppm: 9.49 (s, 1H), 8.69 (s, 1H), 8.16 (s, 1H), 7.61 (s, 1H), 4.21 (d, J = 10.5 Hz, 1H), 4.07-3.98 (m, 2H), 3.65 (d, J = 10.5 Hz, 1H), 2.90 (q, J = 7.5 Hz, 2H), 2.90-2.88 (m, 1H), 2.32-2.16 (m, 7H), 1.66 (s, 3H), 1.38 (t, J = 7.5 Hz, 3H). |
| 361 | N-(3-(3-cyano-3-ethylpyrrolidin-1-yl)-1-(2-(1,1-difluoroethyl)-6- ethylpyrimidin-4-yl)-1H-pyrazolo[4,3-c]pyridin-6-yl)acetamide 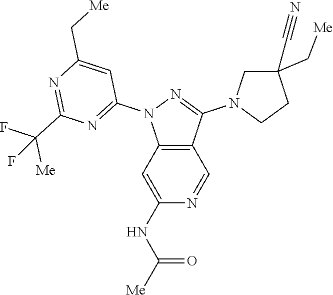 [SMILES: CCc1cc(-n2nc(N3CCC(C#N)(CC)C3)c3cnc(NC(C)=O)cc32)nc(C(C)(F)F)n1] SM: 3-ethylpyrrolidine-3-carbonitrile hydrochloride and Bromo pyrazolo[4,3- c]pyridine 4 prep-HPLC-C (36% to 66% MeCN). 33.0 mg, 30.0% yield as a white solid. LCMS m/z = 469.2 [M + H]⁺ 1H NMR (500 MHz, CDCl₃) δ ppm: 9.51 (s, 1H), 8.71 (s, 1H), 8.14 (s, 1H), 7.62 (s, 1H), 4.21 (d J = 10.5 Hz, 1H), 4.07-3.99 (m, 2H), 3.65-3.62 (m, 1H), 2.90 (q, J = 7.0 Hz, 2H), 2.90-2.86 (m, 1H), 2.32-2.27 (m, 6H), 2.27-2.22 (m, 1H), 1.90-1.88 (m, 2H), 1.38 (t, J = 7.5 Hz, 3H), 1.26 (t, J = 7.5 Hz, 3H). |
| 362 | N-(3-(3-cyanopyrrolidin-1-yl)-1-(2-(1,1-difluoroethyl)-6-ethylpyrimidin-4-yl)- 1H-pyrazolo[4,3-c]pyridin-6-yl)acetamide 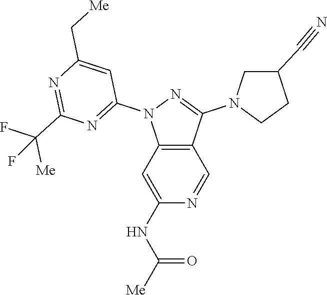 [SMILES: CCc1cc(-n2nc(N3CCC(C#N)C3)c3cnc(NC(C)=O)cc32)nc(C(C)(F)F)n1] SM: pyrrolidine-3-carbonitrile hydrochloride and Bromo pyrazolo[4,3- c]pyridine 4 prep-HPLC-C (34% to 63% MeCN). 55.72 mg, 53.8% yield as a white solid. LCMS m/z = 441.3 [M + H]⁺ 1H NMR 500 MHz, CDCl₃) δ ppm: 9.66 (s, 1H), 8.70 (s, 1H), 7.66 (s, 1H), 4.09-4.00 (m, 3H), 3.93-3.88 (m, 1H), 3.39 (t, J = 5.0 Hz, 1H), 2.94 (q, J = 7.5 Hz, 2H), 2.59-2.51 (m, 2H), 2.32-2.23(m, 6H), 1.39 (t, J = 7.5 Hz, 3H). |

### Example 363: N-(3-(2-(benzyloxy)cyclopropyl)-1-(4-(1,1-difluoroethyl)pyrimidin-2-yl)-1H-pyrazolo[4,3-c]pyridin-6-yl)acetamide

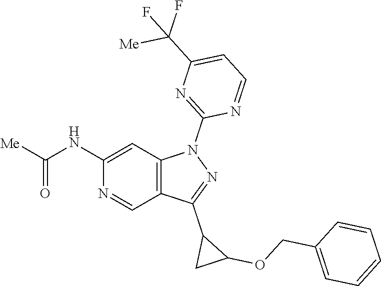 [SMILES: CC(=O)Nc1cc2c(cn1)c(C1CC1OCc1ccccc1)nn2-c1nccc(C(C)(F)F)n1]

To a solution of N-(1-(4-(1,1-difluoroethyl)pyrimidin-2-yl)-3-iodo-1H-pyrazolo[4,3-c]pyridin-6-yl)acetamide (Preparation 506, 500 mg, 1.13 mmol) and 2-(2-(benzyloxy)cyclopropyl)-4,4,5,5-tetramethyl-1,3,2-dioxaborolane (462.9 mg, 1.69 mmol) in H₂O (1 mL) and toluene (10 mL) was added Cs₂CO₃ (1.1 g, 3.38 mmol) and CatacxiumA-Pd-G₂ (75.3 mg, 0.113 mmol) at 25° C. The reaction was stirred at 80° C. for 16 h under N₂. The mixture was concentrated and purified by column chromatography (PE/EtOAc=5/1 to 0/1) on silica gel. The product was purified by prep-HPLC-U (51% to 71% MeCN) to give N-(3-(2-(benzyloxy)cyclopropyl)-1-(4-(1,1-difluoroethyl)pyrimidin-2-yl)-1H-pyrazolo[4,3-c]pyridin-6-yl)acetamide (6.0 mg, 1.2% yield) as a white solid. LCMS m/z=465.2 [M+H]⁺. ¹H NMR: (500 MHz, CDCl₃) δ ppm: 9.41 (s, 1H), 9.00 (d, J=5.0 Hz, 1H), 8.70 (s, 1H), 8.57 (s, 1H), 7.49 (d, J=5.0 Hz, 1H), 7.34-7.30 (m, 5H), 4.73-4.63 (m, 2H), 3.94-3.91 (m, 1H), 2.51-2.49 (m, 1H), 2.32-2.24 (m, 6H), 1.72-1.71 (m, 1H), 1.60-1.57 (m, 1H).

### Example 364: N-(3-(2-cyanocyclopropyl)-1-(4-(1,1-difluoroethyl)pyrimidin-2-yl)-1H-pyrazolo[4,3-c]pyridin-6-yl)acetamide

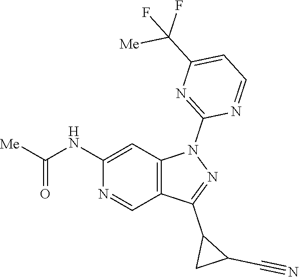 [SMILES: CC(=O)Nc1cc2c(cn1)c(C1CC1C#N)nn2-c1nccc(C(C)(F)F)n1]

N-(3-(2-Cyanocyclopropyl)-1-(4-(1,1-difluoroethyl)pyrimidin-2-yl)-1H-pyrazolo[4,3-c]pyridin-6-yl)acetamide was obtained as a yellow solid, 6 mg, 3.5% yield, from N-(1-(4-(1,1-difluoroethyl)pyrimidin-2-yl)-3-iodo-1H-pyrazolo[4,3-c]pyridin-6-yl)acetamide (Preparation 506) and 2-(4,4,5,5-tetramethyl-1,3,2-dioxaborolan-2-yl)cyclopropane-1-carbonitrile (Preparation 375), following a similar procedure to that described in Example 363. LCMS m/z=384.1 [M+H]^{+ 1}H NMR (400 MHz, MeOD-d₄) δ ppm: 9.40 (s, 1H), 9.07 (d, J=5.2 Hz, 1H), 9.04 (d, J=0.8 Hz, 1H), 7.68 (d, J=5.2 Hz, 1H), 3.27-3.25 (m, 1H), 2.53-2.51 (m, 1H), 2.31-2.21 (m, 6H) 1.97-1.95 (m, 1H), 1.84-1.87 (m, 1H).

### Example 365: N-(3-(6-oxa-3-azabicyclo[3.1.1]heptan-3-yl)-1-(2-(1,1-difluoroethyl)-6-isopropylpyrimidin-4-yl)-1H-pyrazolo[4,3-c]pyridin-6-yl)acetamide

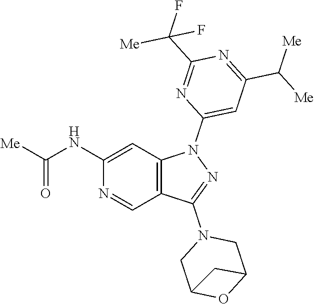 [SMILES: CC(=O)Nc1cc2c(cn1)c(N1CC3CC(C1)O3)nn2-c1cc(C(C)C)nc(C(C)(F)F)n1]

To a solution of N-(3-(6-oxa-3-azabicyclo[3.1.1]heptan-3-yl)-1H-pyrazolo[4,3-c]pyridin-6-yl)acetamide (Preparation 543, 150 mg, 0.55 mmol) in dioxane (5 mL) was added 4-chloro-2-(1,1-difluoroethyl)-6-isopropylpyrimidine (Preparation 451, 145.3 mg, 0.66 mmol), t-BuBrettphos G₃ Pd (46.9 mg, 0.055 mmol) and Cs₂CO₃ (894.2 mg, 2.74 mmol) and the reaction was stirred at 100° C. for 4 h under N₂. The mixture was concentrated under vacuum to give the crude, which was purified by prep-HPLC-Q (48% to 68% MeCN) to give N-(3-(6-oxa-3-azabicyclo[3.1.1]heptan-3-yl)-1-(2-(1,1-difluoroethyl)-6-isopropylpyrimidin-4-yl)-1H-pyrazolo[4,3-c]pyridin-6-yl)acetamide (60.0 mg, 23.9% yield) as a yellow solid. LCMS m/z=458.2 [M+H]⁺. ¹H NMR (400 MHz, DMSO-d₆) δ ppm: 10.75 (s, 1H), 9.41 (s, 1H), 9.04 (s, 1H), 7.70 (s, 1H), 4.76 (d, J=6.4 Hz, 2H), 4.05-4.02 (m, 2H), 3.96-3.93 (m, 2H), 3.24-3.20 (m, 1H), 3.14-3.07 (m, 1H), 2.28-2.18 (m, 3H), 2.15 (s, 3H), 2.04-2.02 (m, 1H), 1.29 (d, J=6.8 Hz, 2H).

Examples 366 to 370: The compounds in the following table were prepared from N-(3-(2-oxa-5-azabicyclo[2.2.1]heptan-5-yl)-1H-pyrazolo[4,3-c]pyridin-6-yl)acetamide hydrochloride (Preparation 561) and the appropriate aryl halide, following the procedure described in Example 365.

| Example | |
|---|---|
| No | Name, Structure, Starting Halide, Data |
| 366 | N-(3-(2-oxa-5-azabicyclo[2.2.1]heptan-5-yl)-1-(6-(1,1-difluoroethyl)pyrazin- 2-yl)-1H-pyrazolo[4,3-c]pyridin-6-yl)acetamide 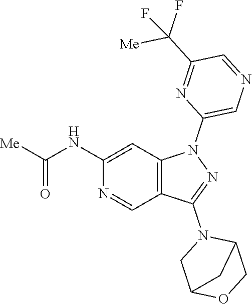 [SMILES: CC(=O)Nc1cc2c(cn1)c(N1CC3CC1CO3)nn2-c1cncc(C(C)(F)F)n1] Halide: 2-chloro-6-(1,1-difluoroethyl)pyrazine prep-HPLC-P (30% to 60% MeCN) 10.0 mg, 16.7% yield as a white solid. LCMS m/z = 416.1 [M + H]⁺. ¹H NMR (400 MHz, CDCl₃) δ ppm: 9.34 (s, 1H), 9.27 (s, 1H), 8.69-8.66 (m, 2H), 8.09 (s, 1H), 4.82-4.77 (m, 2H), 4.15-4.13 (m, 1H), 3.96-3.88 (m, 2H), 3.75-3.72 (m, 1H), 2.36-2.26 (m, 6H), 2.12-2.04 (m, 2H). |
| 367 | N-(3-(2-oxa-5-azabicyclo[2.2.1]heptan-5-yl)-1-(2-(1,1-difluoroethyl)- 6-isopropylpyrimidin-4-yl)-1H-pyrazolo[4,3-c]pyridin-6-yl)acetamide 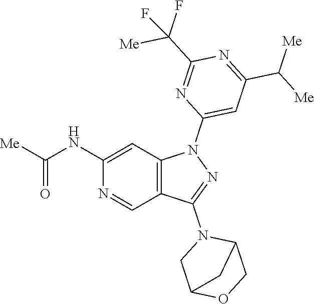 [SMILES: CC(=O)Nc1cc2c(cn1)c(N1CC3CC1CO3)nn2-c1cc(C(C)C)nc(C(C)(F)F)n1] Halide: 4-chloro-2-(1,1-difluoroethyl)-6-isopropylpyrimidine (Preparation 451) prep-HPLC-A (37% to 67% MeCN) 50 mg, 33.9% yield as a white solid. LCMS m/z = 458.3 [M + H]⁺ ¹H NMR (400 MHz, CDCl₃) δ ppm: 9.50 (s, 1H), 8.64 (d, 1H), 8.13 (s, 1H), 7.60 (s, 1H), 4.84 (s, 1H), 4.77 (s, 1H), 4.12 (d, J = 7.6 Hz, 1H), 3.96 (d, J = 6.4 Hz, 1H), 3.88 (t, J = 11.8 Hz, 1H), 3.75 (d, J = 9.2 Hz, 1H), 3.15-3.10 (m, 1H), 2.32-2.22 (m, 6H), 2.11-2.04 (m, 2H), 1.37 (d, J = 7.2 Hz, 6H). |
| 368 | N-(3-(2-oxa-5-azabicyclo[2.2.1]heptan-5-yl)-1-(6-(tetrahydrofuran-3- yl)pyridin-2-yl)-1H-pyrazolo[4,3-c]pyridin-6-yl)acetamide 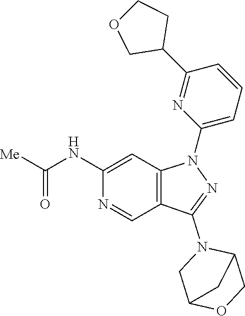 [SMILES: CC(=O)Nc1cc2c(cn1)c(N1CC3CC1CO3)nn2-c1cccc(C2CCOC2)n1] Halide: 2-bromo-6-(tetrahydrofuran-3-yl)pyridine prep-HPLC-A (21% to 51% MeCN). 45.0 mg, 33.2% yield as a white solid. LCMS m/z = 421.2 [M + H]⁺. ¹H NMR: (400 MHz, CDCl₃) δ ppm: 9.74 (s, 1H), 8.97 (s, 1H), 8.46 (br s, 1H), 8.04 (s, 1H), 8.02 (s, 1H), 7.35-7.31 (m, 1H), 5.13 (s, 1H), 5.08 (s, 1H), 4.68-4.64 (m, 1H), 4.61-4.55 (m, 1H), 4.50-4.45 (m, 1H), 4.40-4.33 (m, 2H), 4.28 (d, J = 8.0 Hz, 1H), 4.20 (d, J = 9.2 Hz, 1H), 4.06-3.95 (m, 2H), 2.87-2.83 (m, 2H), 2.59 (s, 3H), 2.44-2.38 (m, 2H). |
| 369 | N-(3-(2-oxa-5-azabicyclo[2.2.1]heptan-5-yl)-1-(6-(1,1-difluoroethyl)- 4-((1s,3s)-3-methoxycyclobutoxy)pyridin-2-yl)-1H-pyrazolo[4,3-c]pyridin-6- yl)acetamide 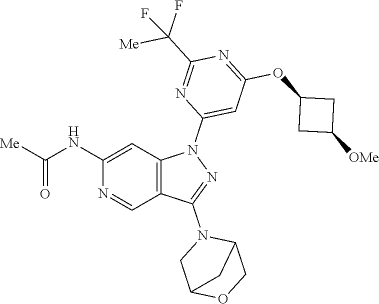 [SMILES: CO[C@H]1C[C@@H](Oc2cc(-n3nc(N4CC5CC4CO5)c4cnc(NC(C)=O)cc43)nc(C(C)(F)F)n2)C1] Halide: 2-chloro-6-(1,1-difluoroethyl)-4-((1s,3s)-3- methoxycyclobutoxy)pyridine (Preparation 480) prep-HPLC-A (40% to 60% MeCN). 55.0 mg, 25.5% yield as a white solid. LCMS m/z = 515.2 [M + H]⁺. ¹H NMR: (400 MHz, CDCl₃) δ ppm: 9.36 (s, 1H), 8.62 (s, 1H), 8.05 (br s, 1H), 7.32 (d, J = 2.0 Hz, 1H), 6.91-6.89 (m, 1H), 4.77 (d, J = 13.2 Hz, 2H), 4.52-4.50 (m, 1H), 4.14-4.12 (m, 1H), 3.94-3.92 (m, 1H), 3.87-3.85 (m, 1H), 3.72-3.71 (m, 2H), 3.28 (s, 3H), 2.95-2.94 (m, 2H), 2.33-2.21 (m, 6H) 2.08-2.05 (m, 4H). |
| 370 | N-(3-(2-oxa-5-azabicyclo[2.2.1]heptan-5-yl)-1-(6-(1,2- difluoroethyl)pyridin-2-yl)-1H-pyrazolo[4,3-c]pyridin-6-yl)acetamide 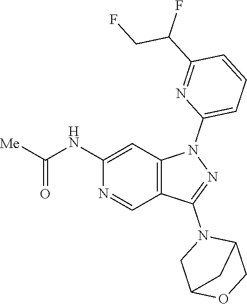 [SMILES: CC(=O)Nc1cc2c(cn1)c(N1CC3CC1CO3)nn2-c1cccc(C(F)CF)n1] Halide: 2-bromo-6-(1,2-difluoroethyl)pyridine (Preparation 437) 16.0 mg, 8.0% yield as white solid. LCMS m/z = 415.1 [M + H]⁺ ¹H NMR (400 MHz, CDCl₃) δ ppm: 9.36 (s, 1H), 8.64 (s, 1H), 8.04 (br s, 1H), 7.87-7.80 (m, 2H), 7.32 (d, J = 6.8 Hz, 1H), 5.96-5.78 (m, 1H), 5.32-5.13 (m, 2H), 4.80-4.77 (m, 2H), 4.15-4.12 (m, 1H), 3.94-3.86 (m, 2H) 3.73-3.70 (m, 1H), 2.26 (s, 3H), 2.10-2.01 (m, 2H). |

### Example 371: N-(3-(6-oxa-3-azabicyclo[3.1.1]heptan-3-yl)-1-(2-(1,1-difluoroethyl)-6-ethylpyrimidin-4-yl)-1H-pyrazolo[4,3-c]pyridin-6-yl)propionamide

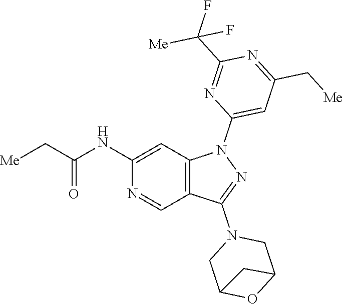 [SMILES: CCC(=O)Nc1cc2c(cn1)c(N1CC3CC(C1)O3)nn2-c1cc(CC)nc(C(C)(F)F)n1]

N-(3-(6-oxa-3-azabicyclo[3.1.1]heptan-3-yl)-1-(2-(1,1-difluoroethyl)-6-ethylpyrimidin-4-yl)-1H-pyrazolo[4,3-c]pyridin-6-yl)propionamide was obtained, 45 mg, 46.9% as a white solid, from N-(3-(6-oxa-3-azabicyclo[3.1.1]heptan-3-yl)-1H-pyrazolo[4,3-c]pyridin-6-yl)propionamide (Preparation 545) and 4-chloro-2-(1,1-difluoroethyl)-6-ethylpyrimidine (Preparation 390) following a similar procedure to that described in Example 365. LCMS m/z=458.1 [M+H]⁺. ¹H NMR (500 MHz, DMSO-d₆) δ ppm 10.69 (s, 1H), 9.42 (s, 1H), 9.04 (s, 1H), 7.70 (s, 1H), 4.77 (d, J=6.5 Hz, 2H), 4.02 (d, J=11.5 Hz, 2H), 3.94 (d, J=11.5 Hz, 2H), 3.23-3.22 (m, 1H), 2.88-2.84 (m, 2H), 2.46-2.43 (m, 2H), 2.24 (t, J=19.0 Hz, 3H), 2.04-2.02 (m, 1H), 1.28 (t, J=7.5 Hz, 3H), 1.11 (t, J=7.5 Hz, 3H).

### Example 372: methyl (3-(6-oxa-3-azabicyclo[3.1.1]heptan-3-yl)-1-(2-(1,1-difluoroethyl)-6-ethylpyrimidin-4-yl)-1H-pyrazolo[4,3-c]pyridin-6-yl)carbamate

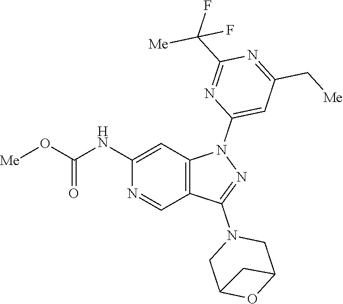 [SMILES: CCc1cc(-n2nc(N3CC4CC(C3)O4)c3cnc(NC(=O)OC)cc32)nc(C(C)(F)F)n1]

To a solution of methyl (3-(6-oxa-3-azabicyclo[3.1.1]heptan-3-yl)-1H-pyrazolo[4,3-c]pyridin-6-yl)carbamate (Preparation 544, 100 mg, 0.346 mmol) in dioxane (5 mL) was added 4-chloro-2-(1,1-difluoroethyl)-6-ethylpyrimidine (Preparation 390, 107.1 mg, 0.519 mmol), Cs₂CO₃ (337.8 mg, 1.04 mmol) and t-BuBrettphos G3 (29.5 mg, 0.035 mmol) and the reaction mixture was stirred at 100° C. for 12 h under N2. The mixture was treated with H₂O (30 mL) and extracted with DCM (30 mL×3). The organic phase was washed with brine (30 mL), dried with Na₂SO₄, filtered and concentrated in vacuo. The residue was poured into MeOH (5 mL), the mixture was stirred at 20° C. for 10 min, filtered the filter cake was washed with EtOAc (2 mL×2) and concentrated in vacuo. The crude was purified by prep-TLC (EtOAc) to give methyl (3-(6-oxa-3-azabicyclo[3.1.1]heptan-3-yl)-1-(2-(1,1-difluoroethyl)-6-ethylpyrimidin-4-yl)-1H-pyrazolo[4,3-c]pyridin-6-yl)carbamate (12 mg, 7.6% yield) as a white solid. LCMS m/z=460.1 [M+H]⁺. 1H NMR: (500 MHz, CDCl₃) δ ppm: 9.30 (s, 1H), 8.82 (s, 1H), 7.84 (br s, 1H), 7.66 (s, 1H), 4.84 (d, J=6.5 Hz, 2H), 4.11-4.08 (m, 2H), 4.01-3.98 (m, 2H), 3.87 (s, 3H), 3.41-3.36 (m, 1H), 2.94-2.88 (m, 2H), 2.26 (t, J=19.0 Hz, 3H), 2.14 (d, J=9.0 Hz, 1H), 1.39 (t, J=7.5 Hz, 3H).

### Example 373 N-(3-(6-oxa-3-azabicyclo[3.1.1]heptan-3-yl)-1-(2-(1,1-difluoroethyl)-6-ethylpyrimidin-4-yl)-1H-pyrazolo[4,3-c]pyridin-6-yl)formamide

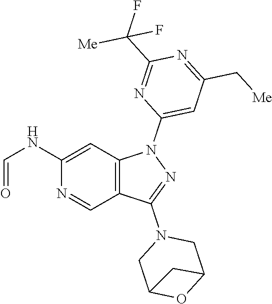 [SMILES: CCc1cc(-n2nc(N3CC4CC(C3)O4)c3cnc(NC=O)cc32)nc(C(C)(F)F)n1]

N-(3-(6-Oxa-3-azabicyclo[3.1.1]heptan-3-yl)-1-(2-(1,1-difluoroethyl)-6-ethylpyrimidin-4-yl)-1H-pyrazolo[4,3-c]pyridin-6-yl)formamide was obtained, 38 mg, 10.4% yield as a white solid, from

N-(3-(6-oxa-3-azabicyclo[3.1.1]heptan-3-yl)-1H-pyrazolo[4,3-c]pyridin-6-yl)formamide hydrochloride (Preparation 560) and 4-chloro-2-(1,1-difluoroethyl)-6-ethylpyrimidine (Preparation 390) following a similar procedure to that described in Example 372. LCMS m/z=430.1 [M+H]⁺. ¹H NMR (400 MHz, DMSO-d₆) δ pp: 10.76 (br s, 1H), 9.04 (s, 1H), 7.71 (s, 1H), 4.77 (br d, J=6.4 Hz, 2H), 4.05-3.95 (m, 4H), 3.26-3.21 (m, 1H), 2.90-2.84 (m, 2H), 2.20 (t, J=19.2 Hz, 3H), 2.15-2.04 (m, 1H), 1.31 (t, J=7.6 Hz, 3H).

### Example 374: N-(3-(6-oxa-3-azabicyclo[3.1.1]heptan-3-yl)-1-(6-(1,2-difluoroethyl)pyridin-2-yl)-1H-pyrazolo[4,3-c]pyridin-6-yl)acetamide

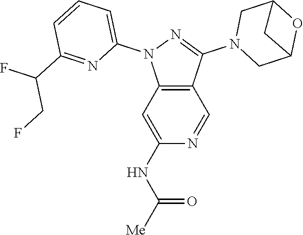 [SMILES: CC(=O)Nc1cc2c(cn1)c(N1CC3CC(C1)O3)nn2-c1cccc(C(F)CF)n1]

To a solution of N-(3-(6-oxa-3-azabicyclo[3.1.1]heptan-3-yl)-1H-pyrazolo[4,3-c]pyridin-6-yl)acetamide (Preparation 543, 50 mg, 0.183 mmol) and 2-bromo-6-(1,2-difluoroethyl)pyridine (Preparation 437, 44.68 mg, 0.20 mmol) in dioxane (5 mL) was added Brettphos Pd G₃ (23.45 mg, 27.44 μmol) and Cs₂CO₃ (178.8 mg, 0.55 mmol) and the reaction was stirred at 100° C. for 5 h under N₂. The mixture was concentrated under vacuum and diluted with EtOAc (30 mL). The mixture was filtered and concentrated under vacuum and the crude was purified by prep-HPLC-A (30% to 60% MeCN) to give N-(3-(6-oxa-3-azabicyclo[3.1.1]heptan-3-yl)-1-(6-(1,2-difluoroethyl)pyridin-2-yl)-1H-pyrazolo[4,3-c]pyridin-6-yl)acetamide (23 mg, 30.3% yield) as a white solid. LCMS m/z=415.1 [M+H]^{+ 1}HNMR: (400 MHz, CDCl₃) δ ppm: 9.43 (s, 1H), 8.80 (m, 1H), 8.25 (br s, 1H), 7.85 (d, J=4.8 Hz, 2H), 7.34-7.33 (m, 1H), 5.98-5.79 (m, 1H), 5.32-5.13 (m, 2H), 4.83 (d, J=6.4 Hz, 2H), 4.09-3.97 (m, 4H), 3.40-3.35 (m, 1H), 2.27 (s, 3H), 2.16 (d, J=9.2 Hz, 1H).

### Example 375: N-(3-(6-oxa-3-azabicyclo[3.1.1]heptan-3-yl)-1-(6-(1,1-difluoroethyl)pyrazin-2-yl)-1H-pyrazolo[4,3-c]pyridin-6-yl)acetamide

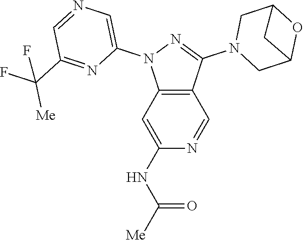 [SMILES: CC(=O)Nc1cc2c(cn1)c(N1CC3CC(C1)O3)nn2-c1cncc(C(C)(F)F)n1]

N-(3-(6-Oxa-3-azabicyclo[3.1.1]heptan-3-yl)-1-(6-(1,1-difluoroethyl)pyrazin-2-yl)-1H-pyrazolo[4,3-c]pyridin-6-yl)acetamide was obtained as a white solid, 15.8 mg, 19.8%, from N-(3-(6-oxa-3-azabicyclo[3.1.1]heptan-3-yl)-1H-pyrazolo[4,3-c]pyridin-6-yl)acetamide (Preparation 543) and 2-chloro-6-(1,1-difluoroethyl)pyrazine, following a similar procedure to that described in Example 374. LCMS m/z=416.1 [M+H]⁺. ¹H NMR (400 MHz, CDCl₃) δ ppm: 9.38 (s, 1H), 9.31 (s, 1H), 8.84 (s, 1H), 8.69 (s, 1H), 8.14 (s, 1H), 4.84 (d, J=6.8 Hz, 2H), 4.12-3.99 (m, 4H), 3.42-3.36 (m, 1H), 2.37-2.27 (m, 6H), 2.17-2.14 (m, 1H).

Examples 376 to 379: The compounds in the following table were prepared from N-(3-(2-oxa-5-azabicyclo[2.2.1]heptan-5-yl)-1H-pyrazolo[4,3-c]pyridin-6-yl)acetamide hydrochloride (Preparation 561) and the appropriate aryl halide, following a similar procedure to that described in Example 374.

| Example No | Name, Structure, Starting halide, Data |
|---|---|
| 376 | N-(3-(2-oxa-5-azabicyclo[2.2.1]heptan-5-yl)-1-(6-(2-fluoropropan-2-yl)-4- methoxypyridin-2-yl)-1H-pyrazolo[4,3-c]pyridin-6-yl)acetamide 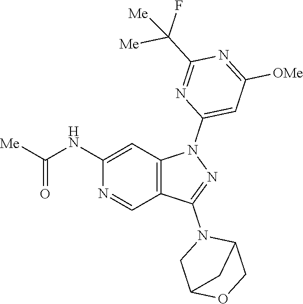 [SMILES: COc1cc(-n2nc(N3CC4CC3CO4)c3cnc(NC(C)=O)cc32)nc(C(C)(C)F)n1] Halide: 2-bromo-6-(2-fluoropropan-2-yl)-4-methoxypyridine (Preparation 435) prep-HPLC-R (39% to 69% MeCN). 37.4 mg, 23.2% yield as a white solid. LCMS m/z = 441.2 [M + H]⁺ ¹H NMR: (400 MHz, CDCl₃) δ ppm: 9.36 (s, 1H), 8.62 (s, 1H), 8.02 (s, 1H), 7.25 (s, 1H), 6.92-6.90 (m, 1H), 4.80-4.73 (m, 2H), 4.15-4.12 (m, 1H), 3.95 (s, 3H), 3.93-3.91 (m, 1H), 3.88-3.84 (m, 1H), 3.73-3.70 (m, 1H), 2.26 (s, 3H), 2.10-2.00 (m, 2H), 1.93-1.91 (m, 3H), 1.87-1.85 (m, 3H). |
| 377 | N-(3-(2-oxa-5-azabicyclo[2.2.1]heptan-5-yl)-1-(6-(1,1-difluoroethyl)-4- methoxypyridin-2-yl)-1H-pyrazolo[4,3-c]pyridin-6-yl)acetamide 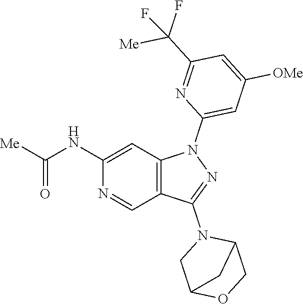 [SMILES: COc1cc(-n2nc(N3CC4CC3CO4)c3cnc(NC(C)=O)cc32)nc(C(C)(F)F)c1] Halide: 2-bromo-6-(1, 1-difluoroethyl)-4-methoxypyridine (Preparation 436) prep-HPLC-A (30% to 60% MeCN). 22 mg, 17.8% yield as a white solid. LCMS m/z = 445.2 [M + H]⁺. ¹H NMR (400 MHz, CDCl₃). δ ppm: 9.38 (s, 1H), 8.63 (s, 1H), 8.06 (br s, 1H), 7.39 (d, J = 2.0 Hz, 1H), 7.01 (d, J = 2.0 Hz, 1H), 4.80 (s, 1H), 4.75 (s, 1H), 4.13 (d, J = 7.6 Hz, 1H), 3.97 (s, 3H), 3.94- 3.92 (m, 1H), 3.88-3.85 (m, 1H), 3.72 (d, J = 8.8 Hz, 1H), 2.33-2.24 (m, 6H), 2.10-2.01 (m, 2H). |
| 378 | N-(3-(2-oxa-5-azabicyclo[2.2.1]heptan-5-yl)-1-(6-(1,1-difluoroethyl)-4- ((1r,3r)-3-methoxycyclobutoxy)pyridin-2-yl)-1H-pyrazolo[4,3-c]pyridin-6- yl)acetamide 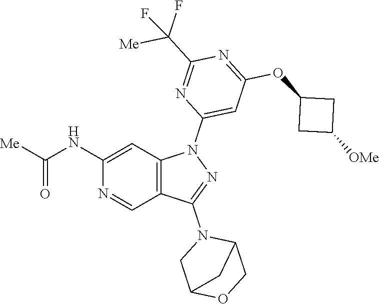 [SMILES: CO[C@H]1C[C@H](Oc2cc(-n3nc(N4CC5CC4CO5)c4cnc(NC(C)=O)cc43)nc(C(C)(F)F)n2)C1] Halide: 2-chloro-6-(1,1-difluoroethyl)-4-((1r,3r)-3- methoxycyclobutoxy)pyridine (Preparation 481) prep-HPLC-A (42% to 72% MeCN). 60.0 mg, 24.1% yield as a white solid. LCMS m/z = 515.2 [M + H]⁺. ¹H NMR (400 MHz, CDCl₃) δ ppm: 9.37 (s, 1H), 8.62 (s, 1H), 8.11 (s, 1H), 7.28 (s, 1H), 6.89 (s, 1H), 5.03-5.01 (m, 1H), 4.77 (d, J = 14.4 Hz, 2H), 4.15-4.11 (m, 2H), 3.94-3.90 (m, 1H), 3.88-3.86 (m, 1H), 3.73-3.70 (m, 1H), 3.30 (s, 3H), 2.55-2.50 (m, 4H), 2.32-2.23 (m, 6H), 2.08-2.07 (m, 2H). |
| 379 | N-(3-(2-oxa-5-azabicyclo[2.2.1]heptan-5-yl)-1-(6-(1,1-difluoroethyl)-4-(2- methoxyethoxy)pyridin-2-yl)-1H-pyrazolo[4,3-c]pyridin-6-yl)acetamide 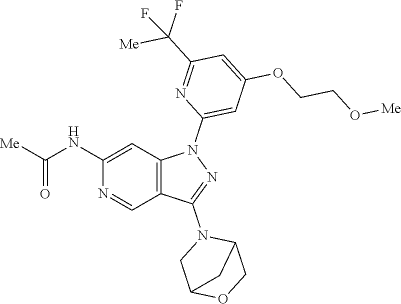 [SMILES: COCCOc1cc(-n2nc(N3CC4CC3CO4)c3cnc(NC(C)=O)cc32)nc(C(C)(F)F)c1] Halide: 4-chloro-2-(1,1-difluoroethyl)-6-(2-methoxyethoxy)pyrimidine (Preparation 474) prep-HPLC-S (28% to 58% MeCN). 20.3 mg, 27.5% yield as a white solid. LCMS m/z = 489.0 [M + H]⁺. ¹H NMR (400 MHz, DMSO-d₆) δ ppm 10.65 (s, 1H), 9.30 (s, 1H), 8.90 (s, 1H), 7.40 (d, J = 2.0 Hz, 1H), 7.04 (d, J = 2.0 Hz, 1H), 4.92 (s, 1H), 4.71 (s, 1H), 4.37-4.32 (m, 2H), 3.87-3.81 (m, 3H), 3.72-3.69 (m, 2H), 3.62-3.60 (m, 1H), 3.32 (s, 3H), 2.22 (t, J = 19.6 Hz, 3H), 2.14 (s, 3H), 2.03-1.91 (m, 2H). |

### Example 380: N-(3-cyclopropyl-1-(2-(1,1-difluoroethyl)-5-fluoro-6-methylpyrimidin-4-yl)-1H-pyrazolo[4,3-c]pyridin-6-yl)acetamide

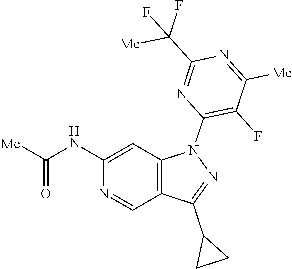 [SMILES: CC(=O)Nc1cc2c(cn1)c(C1CC1)nn2-c1nc(C(C)(F)F)nc(C)c1F]

To a solution of N-(3-cyclopropyl-1H-pyrazolo[4,3-c]pyridin-6-yl)acetamide (Preparation 11, 60.0 mg, 0.277 mmol) in DMF (2 mL) was added 4-chloro-2-(1,1-difluoroethyl)-5-fluoro-6-methylpyrimidine (Preparation 463, 70.1 mg, 0.333 mmol) and Cs₂CO₃ (271.2 mg, 0.832 mmol) and the reaction was stirred at 70° C. for 2 h. The mixture was concentrated and purified by prep-HPLC-C (36% to 56% MeCN) to give N-(3-cyclopropyl-1-(2-(1,1-difluoroethyl)-5-fluoro-6-methylpyrimidin-4-yl)-1H-pyrazolo[4,3-c]pyridin-6-yl)acetamide (9.5 mg, 8.8% yield) as a white solid. LCMS m/z=391.1 [M+H]^{+ 1}H NMR (500 MHz, CDCl₃) δ ppm: 9.28 (s, 1H), 8.76 (s, 1H), 8.24 (s, 1H), 2.68 (s, 3H), 2.29-2.20 (m, 7H), 1.24-1.17 (m, 4H).

### Example 381: N-(3-cyclopropyl-1-(2-(1,1-difluoroethyl)-5-fluoropyrimidin-4-yl)-1H-pyrazolo[4,3-c]pyridin-6-yl)acetamide

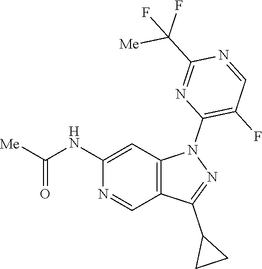 [SMILES: CC(=O)Nc1cc2c(cn1)c(C1CC1)nn2-c1nc(C(C)(F)F)ncc1F]

N-(3-Cyclopropyl-1-(2-(1,1-difluoroethyl)-5-fluoropyrimidin-4-yl)-1H-pyrazolo[4,3-c]pyridin-6-yl)acetamide was obtained as a yellow solid, 14 mg, 13.4% yield, from N-(3-cyclopropyl-1H-pyrazolo[4,3-c]pyridin-6-yl)acetamide (Preparation 11) and 4-chloro-2-(1,1-difluoroethyl)-5-fluoropyrimidine (Preparation 464) following the procedure described in Example 380. LCMS m/z=377.1 [M+H]⁺. ¹H NMR (400 MHz, DMSO-d₆) δ: ppm 10.85 (s, 1H), 9.24 (s, 1H), 9.08-9.05 (m, 2H), 2.51 (s, 1H), 2.26-2.15 (m, 6H), 1.18-1.14 (m, 4H).

### Example 382: N-(1-(6-chloro-2-(1,1-difluoroethyl)pyrimidin-4-yl)-3-(3-(dimethylamino)-3-methylpyrrolidin-1-yl)-1H-pyrazolo[4,3-c]pyridin-6-yl)acetamide

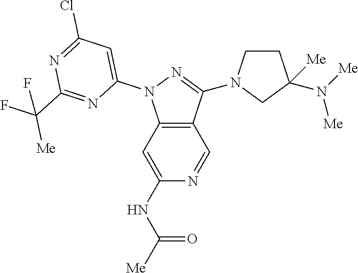 [SMILES: CC(=O)Nc1cc2c(cn1)c(N1CCC(C)(N(C)C)C1)nn2-c1cc(Cl)nc(C(C)(F)F)n1]

A mixture of 4,6-dichloro-2-(1,1-difluoroethyl)pyrimidine (704.5 mg, 3.31 mmol), N-(3-(3-(dimethylamino)-3-methylpyrrolidin-1-yl)-1H-pyrazolo[4,3-c]pyridin-6-yl)acetamide (Preparation 547, 1 g, 3.31 mmol) and Cs₂CO₃ (2.16 g, 6.61 mmol) in dry DMF (8 mL) was stirred at 35° C. for 3 h. Water was added and the solid formed was filtered, washed with water and dried under vacuum to give N-(1-(6-chloro-2-(1,1-difluoroethyl)pyrimidin-4-yl)-3-(3-(dimethylamino)-3-methylpyrrolidin-1-yl)-1H-pyrazolo[4,3-c]pyridin-6-yl)acetamide (981 mg, 61.3% yield) as a yellow solid. LCMS m/z=479.0 [M+H]⁺1H NMR (400 MHz, DMSO-d₆) δ ppm 10.78 (s, 1H), 9.30 (s, 1H), 8.93 (s, 1H), 7.73 (s, 1H), 3.83-3.91 (m, 1H), 3.72-3.82 (m, 1H), 3.67 (br d, J=9.5 Hz, 1H), 3.51 (br d, J=9.5 Hz, 1H), 2.12-2.29 (m, 12H), 1.87-2.10 (m, 2H), 1.07 (s, 3H).

### Example 383 and 384: (R)—N-(1-(6-chloro-2-(1,1-difluoroethyl)pyrimidin-4-yl)-3-(3-(dimethylamino)-3-methylpyrrolidin-1-yl)-1H-pyrazolo[4,3-c]pyridin-6-yl)acetamide and (S)—N-(1-(6-chloro-2-(1,1-difluoroethyl)pyrimidin-4-yl)-3-(3-(dimethylamino)-3-methylpyrrolidin-1-yl)-1H-pyrazolo[4,3-c]pyridin-6-yl)acetamide

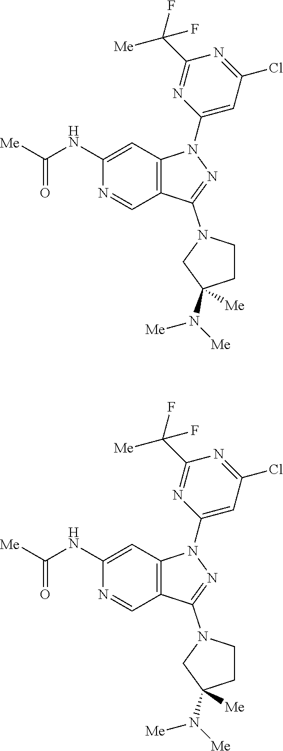 [SMILES: CC(=O)Nc1cc2c(cn1)c(N1CC[C@@](C)(N(C)C)C1)nn2-c1cc(Cl)nc(C(C)(F)F)n1 ; CC(=O)Nc1cc2c(cn1)c(N1CC[C@](C)(N(C)C)C1)nn2-c1cc(Cl)nc(C(C)(F)F)n1]

N-(1-(6-Chloro-2-(1,1-difluoroethyl)pyrimidin-4-yl)-3-(3-(dimethylamino)-3-methylpyrrolidin-1-yl)-1H-pyrazolo[4,3-c]pyridin-6-yl)acetamide (Example 382) was further purified by SFC separation: CHIRALPAK AD-H 30×250 mm, 5 um, Method: 40% EtOH w/0.1% DEA in CO₂ (flow rate: 100 mL/min, ABPR 120bar, MBPR 40 psi, column temp 40 deg C.) to give peak 1, enantiomer 1, (R)—N-(1-(6-chloro-2-(1,1-difluoroethyl)pyrimidin-4-yl)-3-(3-(dimethylamino)-3-methylpyrrolidin-1-yl)-1H-pyrazolo[4,3-c]pyridin-6-yl)acetamide or (S)—N-(1-(6-chloro-2-(1,1-difluoroethyl)pyrimidin-4-yl)-3-(3-(dimethylamino)-3-methylpyrrolidin-1-yl)-1H-pyrazolo[4,3-c]pyridin-6-yl)acetamide.

LCMS m/z=479.1 [M+H]⁺. 1H NMR (500 MHz, DMSO-d₆) δ ppm 10.80 (s, 1H), 9.34 (s, 1H), 8.96 (s, 1H), 7.78 (s, 1H), 3.85-3.95 (m, 1H), 3.74-3.85 (m, 1H), 3.69 (br d, J=9.8 Hz, 1H), 3.53 (br d, J=9.5 Hz, 1H), 2.17-2.27 (m, 9H), 2.16 (s, 3H), 1.88-2.09 (m, 2H), 1.07 (s, 3H).

And peak 2, (S)—N-(1-(6-chloro-2-(1,1-difluoroethyl)pyrimidin-4-yl)-3-(3-(dimethylamino)-3-methylpyrrolidin-1-yl)-1H-pyrazolo[4,3-c]pyridin-6-yl)acetamide or (R)—N-(1-(6-chloro-2-(1,1-difluoroethyl)pyrimidin-4-yl)-3-(3-(dimethylamino)-3-methylpyrrolidin-1-yl)-1H-pyrazolo[4,3-c]pyridin-6-yl)acetamide. LCMS m/z=479.1 [M+H]⁺. 1H NMR (500 MHz, DMSO-d₆) δ ppm 10.80 (s, 1H), 9.34 (s, 1H), 8.96 (s, 1H), 7.78 (s, 1H), 3.85-3.95 (m, 1H), 3.74-3.85 (m, 1H), 3.69 (br d, J=9.8 Hz, 1H), 3.53 (br d, J=9.5 Hz, 1H), 2.17-2.27 (m, 9H), 2.16 (s, 3H), 1.88-2.09 (m, 2H), 1.07 (s, 3H).

### Example 385: N-(1-(6-(1,1-difluoroethyl)pyridin-2-yl)-3-morpholino-1H-pyrazolo[4,3-c]pyridin-6-yl)acetamide

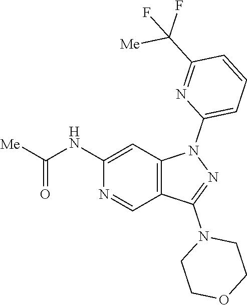 [SMILES: CC(=O)Nc1cc2c(cn1)c(N1CCOCC1)nn2-c1cccc(C(C)(F)F)n1]

To a mixture of N-(3-morpholino-1H-pyrazolo[4,3-c]pyridin-6-yl)acetamide (Preparation 546, 12 mg, 45.9 μmol) in dioxane (1 mL) was added TEA (32 μL, 0.23 mmol) and the solution stirred at rt for 5 min. 2-Bromo-6-(1,1-difluoroethyl)pyridine (16 mg, 68.9 μmol), K₂CO₃ (13 mg, 91.8 μmol), CuI (875 ug, 4.59 μmol) and (1R,2R)—N1,N2-dimethylcyclohexane-1,2-diamine (1.45 μL, 9.19 μmol) were then added. The reaction mixture was purged with N₂ for 2 min then heated at 105° C. for 2 h. The cooled mixture was filtered, and the filtrate was purified by HPLC to give N-(1-(6-(1,1-difluoroethyl)pyridin-2-yl)-3-morpholino-1H-pyrazolo[4,3-c]pyridin-6-yl)acetamide (5 mg, 17% yield). LCMS m/z=403 [M+H]^{+ 1}H NMR (400 MHz, MeOD-d₄) δ 9.00 (br s, 1H), 8.95 (s, 1H), 8.01-8.11 (m, 2H), 7.57 (d, J=7.03 Hz, 1H), 3.86-4.00 (m, 4H), 3.56-3.71 (m, 4H), 2.29 (s, 3H), 2.18 (t, J=18.82 Hz, 3H).

### Example 386: N-(1-(6-(1,1-difluoroethyl)pyridin-2-yl)-3-(methylsulfonyl)-1H-pyrazolo[4,3-c]pyridin-6-yl)acetamide

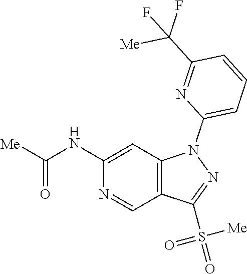 [SMILES: CC(=O)Nc1cc2c(cn1)c(S(C)(=O)=O)nn2-c1cccc(C(C)(F)F)n1]

A mixture of N-(3-(methylsulfonyl)-1H-pyrazolo[4,3-c]pyridin-6-yl)acetamide (Preparation 556, 35 mg, 0.073 mmol), 2-bromo-6-(1,1-difluoroethyl)pyridine (24 mg, 0.109 mmol) and CuI (2 mg, 7.26 μmol) in dioxane (1 mL) was purged with N₂. The reaction was heated at 80° C. for 4 h, cooled to rt and filtered. The filtrate was diluted with EtOAc and washed with brine. The organic layer was dried and concentrated. The crude was purified by HPLC to give N-(1-(6-(1,1-difluoroethyl)pyridin-2-yl)-3-(methylsulfonyl)-1H-pyrazolo[4,3-c]pyridin-6-yl)acetamide (8.3 mg, 28% yield) as a white powder. LCMS m/z=396 [M+H]⁺

### Example 387: (S)—N-(3-(3-cyanopyrrolidin-1-yl)-1-(6-(1,1-difluoroethyl)pyridin-2-yl)-1H-pyrazolo[4,3-c]pyridin-6-yl)acetamide

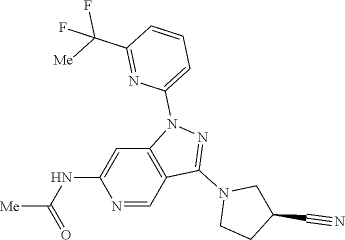 [SMILES: CC(=O)Nc1cc2c(cn1)c(N1CC[C@H](C#N)C1)nn2-c1cccc(C(C)(F)F)n1]

N-(1-(6-(1,1-Difluoroethyl)pyridin-2-yl)-3-(methylsulfonyl)-1H-pyrazolo[4,3-c]pyridin-6-yl)acetamide was obtained 45 mg, 38% from (S)—N-(3-(3-cyanopyrrolidin-1-yl)-1H-pyrazolo[4,3-c]pyridin-6-yl)acetamide trifluoroacetate (Preparation 558) and 2-bromo-6-(1,1-difluoroethyl)pyridine, following a similar procedure to that described in Example 386. LCMS m/z=412 [M+H]⁺1H NMR (500 MHz, DMSO-d₆) δ 10.71 (s, 1H), 9.32 (s, 1H), 8.96 (d, J=0.76 Hz, 1H), 8.05-8.15 (m, 1H), 7.94 (d, J=8.39 Hz, 1H), 7.49 (d, J=7.63 Hz, 1H), 3.98-4.04 (m, 1H), 3.84-3.96 (m, 2H), 3.75-3.83 (m, 1H), 3.65 (quin, J=6.56 Hz, 1H), 2.41-2.48 (m, 1H), 2.31-2.38 (m, 1H), 2.26 (t, J=19.45 Hz, 3H), 2.15 (s, 3H).

### Example 388-: N-(1-(6-(2,2-difluorocyclopropyl)pyridin-2-yl)-1H-pyrazolo[4,3-c]pyridin-6-yl)acetamide

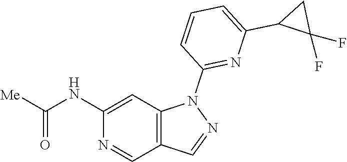 [SMILES: CC(=O)Nc1cc2c(cn1)cnn2-c1cccc(C2CC2(F)F)n1]

To a solution of N-(1-(6-bromopyridin-2-yl)-1H-pyrazolo[4,3-c]pyridin-6-yl)acetamide (Preparation 563, 60 mg, 0.181 mmol) and 2-(2,2-difluorocyclopropyl)-4,4,5,5-tetramethyl-1,3,2-dioxaborolane (74 mg, 0.361 mmol) in dioxane (2 mL) was added aqueous Cs₂CO₃ (2 M, 181 μL), Pd(OAc)₂ (4 mg, 18.1 μmol) and cataCXium A (6.5 mg, 18.1 μmol) and the reaction mixture was heated at 100° C. for 3 h. The cooled mixture was diluted with EtOAc, washed with water (3×), then brine. The organic layer was separated, dried and concentrated. The crude was purified by HPLC to give N-(1-(6-(2,2-difluorocyclopropyl)pyridin-2-yl)-1H-pyrazolo[4,3-c]pyridin-6-yl)acetamide (8 mg, 10% yield, Trifluoroacetic acid) as a white solid. LCMS m/z=330.1 [M+H]⁺; ¹H NMR (400 MHz, CDCl₃) δ 12.30 (br s, 1H), 9.85 (s, 1H), 8.93 (s, 1H), 8.53 (s, 1H), 7.97-8.03 (m, 1H), 7.87-7.95 (m, 1H), 7.33 (d, J=7.53 Hz, 1H), 3.04 (dt, J=8.03, 11.80 Hz, 1H), 2.58-2.72 (m, 1H), 2.42 (s, 3H), 2.00-2.16 (m, 1H)

### Example 389: N-(1-(2-(1,1-difluoroethyl)-6-vinylpyrimidin-4-yl)-3-(3-(dimethylamino)-3-methylpyrrolidin-1-yl)-1H-pyrazolo[4,3-c]pyridin-6-yl)acetamide

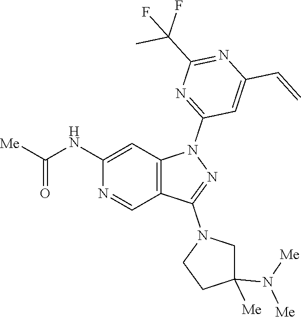 [SMILES: C=Cc1cc(-n2nc(N3CCC(C)(N(C)C)C3)c3cnc(NC(C)=O)cc32)nc(C(C)(F)F)n1]

To a suspension of N-(1-(6-chloro-2-(1,1-difluoroethyl)pyrimidin-4-yl)-3-(3-(dimethylamino)-3-methylpyrrolidin-1-yl)-1H-pyrazolo[4,3-c]pyridin-6-yl)acetamide (Example 382, 950 mg, 1.98 mmol) in MeCN (8 mL) and water (2 mL) was added potassium vinyltrifluoroborate (797.1 mg, 5.95 mmol) and K₂CO₃ (548.3 mg, 3.97 mmol) and the reaction mixture was sparged with N₂ for 5 min. Pd(PPh₃)₄ (45.84 mg, 0.04 mmol) was added and the reaction was heated to reflux for 17 h. The cooled mixture was filtered and the solid washed with water and minimum amount of EtOAc to give 342 mg of desired product. The filtrate was extracted with EtOAc (3×), the combined organic extracts were dried (MgSO₄), filtered and concentrated in vacuo. The residue was purified by silica gel chromatography (0-10% MeOH in DCM) to give N-(1-(2-(1,1-difluoroethyl)-6-vinylpyrimidin-4-yl)-3-(3-(dimethylamino)-3-methylpyrrolidin-1-yl)-1H-pyrazolo[4,3-c]pyridin-6-yl)acetamide as a yellow solid (409 mg). LCMS m/z=471.0 [M+H]⁺ 1H NMR (400 MHz, CDCl₃) δ ppm 9.50 (br s, 1H), 8.72 (d, J=1.0 Hz, 1H), 8.17 (s, 1H), 7.70 (s, 1H), 6.86 (dd, J=18.0 Hz, J=10.5H, 1H), 6.63 (d, J=18.0 Hz, 1H), 5.75 (dd, J=10.5, 1.0 Hz, 1H), 3.95 (td, J=9.5, 2.0 Hz, 1H), 3.83 (td, J=9.8, 7.0 Hz, 1H), 3.64-3.72 (m, 2H), 2.37 (s, 6H), 2.15-2.35 (m, 6H), 2.00-2.21 (m, 1H), 2.03 (ddd, J=12.0, 7.0, 2.0 Hz, 1H), 1.18 (s, 3H).

### Example 390: N-(1-(2-(1,1-difluoroethyl)pyrimidin-4-yl)-3-(3-(dimethylamino)pyrrolidin-1-yl)-1H-pyrazolo[4,3-c]pyridin-6-yl)acetamide

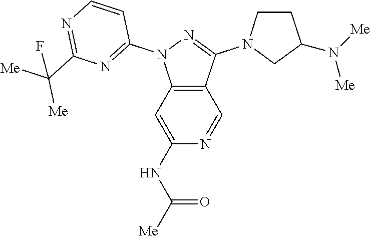 [SMILES: CC(=O)Nc1cc2c(cn1)c(N1CCC(N(C)C)C1)nn2-c1ccnc(C(C)(C)F)n1]

To a solution of 1-(6-chloro-1-(2-(1,1-difluoroethyl)pyrimidin-4-yl)-1H-pyrazolo[4,3-c]pyridin-3-yl)-N,N-dimethylpyrrolidin-3-amine (Preparation 651, 80.0 mg, 0.196 mmol) and acetamide (115.9 mg, 1.96 mmol) in dioxane (3 mL) was added Cs₂CO₃ (127.8 mg, 0.392 mmol), Pd₂(dba)₃ (35.9 mg, 39.2 μmol) and Xantphos (45.4 mg, 78.5 μmol) and the reaction was stirred at 120° C. for 16 h under N₂. The mixture was concentrated under reduced pressure and the residue was purified by prep-HPLC-C (27% to 57%) to give N-(1-(2-(1,1-difluoroethyl)pyrimidin-4-yl)-3-(3-(dimethylamino)pyrrolidin-1-yl)-1H-pyrazolo[4,3-c]pyridin-6-yl)acetamide (20 mg, 23.7% yield) as a white solid. LCMS m/z=431.1 [M+H]^{+ 1}H NMR: (400 MHz, CDCl₃) δ ppm: 9.49 (s, 1H), 8.73 (d, J=5.6 Hz, 2H), 8.12 (s, 1H), 7.79 (d, J=5.6 Hz, 1H), 3.91-3.97 (m, 2H), 3.74-3.76 (m, 1H), 3.52-3.57 (m, 1H), 2.93-2.97 (m, 1H), 2.24-2.37 (m, 13H), 2.04-2.07 (m, 1H).

Examples 391 to 401: The compounds in the following table were prepared from the appropriate 6-chloro-1H-pyrazolo[4,3-c]pyridine and acetamide, following a similar procedure to that described in Example 390.

| Example | |
|---|---|
| No | Name, Structure, Starting Material (SM), Data |
| 391 | N-(1-(2-(1,1-difluoroethyl)pyrimidin-4-yl)-3-(methyl(1-methylpyrrolidin-3- |
| | yl)amino)-1H-pyrazolo[4,3-c]pyridin-6-yl)acetamide |
| | 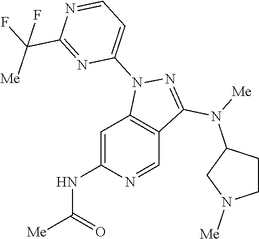 [SMILES: CC(=O)Nc1cc2c(cn1)c(N(C)C1CCN(C)C1)nn2-c1ccnc(C(C)(F)F)n1] |
| | SM: 6-chloro-1-(2-(1,1-difluoroethyl)pyrimidin-4-yl)-N-methyl-N-(1- |
| | methylpyrrolidin-3-yl)-1H-pyrazolo[4,3-c]pyridin-3-amine (Preparation 652) |
| | prep-HPLC-C (28% to 58% MeCN). 3.0 mg, 9.47% yield as a white solid. |
| | LCMS m/z = 431.2 [M + H]⁺ ¹H NMR (400 MHz, CDCl₃) δ ppm: 9.51 (s, 1H), |
| | 8.79 (s, 1H), 8.74 (d, J = 5.6 Hz, 1H), 8.13 (s, 1H), 7.76 (d, J = 5.6 Hz, 1H), 4.93- |
| | 5.01 (m, 1H), 3.26 (s, 3H), 2.79-2.87 (m, 3H), 2.50 (br s, 1H), 2.42 (s, 3H), |
| | 2.26-2.29 (m, 6H), 1.98-2.02 (m, 2H). |
| 392 | N-(1-(2-(1,1-difluoroethyl)pyrimidin-4-yl)-3-(4-(dimethylamino)-2- |
| | methylpyrrolidin-1-yl)-1H-pyrazolo[4,3-c]pyridin-6-yl)acetamide |
| | 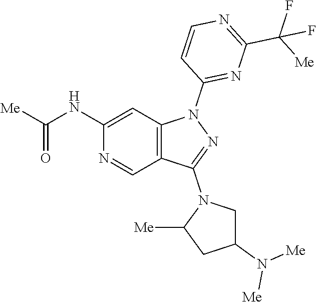 [SMILES: CC(=O)Nc1cc2c(cn1)c(N1CC(N(C)C)CC1C)nn2-c1ccnc(C(C)(F)F)n1] |
| | SM: 1-(6-chloro-1-(2-(1, 1-difluoroethyl)pyrimidin-4-yl)-1H-pyrazolo[4,3- |
| | c]pyridin-3-y1)-N,N,5-trimethylpyrrolidin-3-amine (Preparation 660) |
| | Prep-HPLC-C (30% to 58% MeCN) 5.1 mg, 24.2% yield as a white solid. |
| | LCMS m/z = 445.2 [M + H]⁺ ¹H NMR (500 MHz, CDCl₃) δ ppm: 9.49 (s, 1H), |
| | 8.69-8.74 (m, 2H), 8.11 (s, 1H), 7.76-7.80 (m, 1H), 4.24-4.45 (m, 1H), 3.99- |
| | 4.04 (m, 1H), 3.54-3.71 (m, 1H), 2.78-3.19 (m, 1H), 2.48-2.50 (m, 1H), 2.25- |
| | 2.35 (m, 12H), 1.72-1.97 (m, 1H), 1.38-1.47 (m, 3H). |
| 393 | N-(3-(4-amino-3,3-difluoropyrrolidin-1-yl)-1-(2-(2-fluoropropan-2- |
| | yl)pyrimidin-4-yl)-1H-pyrazolo[4,3-c]pyridin-6-yl)acetamide |
| | 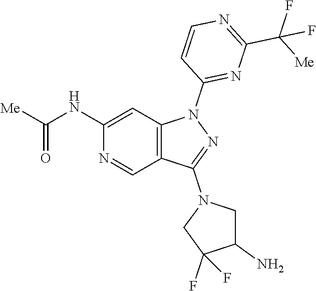 [SMILES: CC(=O)Nc1cc2c(cn1)c(N1CC(N)C(F)(F)C1)nn2-c1ccnc(C(C)(F)F)n1] |
| | SM: 1-(6-chloro-1-(2-(1, 1-difluoroethyl)pyrimidin-4-yl)-1H-pyrazolo[4,3- |
| | c]pyridin-3-yl)-4,4-difluoropyrrolidin-3-amine (Preparation 661) |
| | Prep-HPLC-I (13% to 33% MeCN). 14.2 mg, 16.8% yield as a yellow solid. |
| | LCMS m/z = 439.1 [M + H]⁺ ¹H NMR (500 MHz, DMSO-d₆) δ ppm: 10.84 (s, |
| | 1H), 9.41 (s, 1H), 9.20 (br s, 3H), 8.99 (s, 1H), 8.89 (d, J-6.0 Hz, 1H), 7.83 (d, |
| | J = 5.5 Hz, 1H), 4.41-4.53 (m, 3H), 4.27-4.29 (m, 1H), 3.95-3.99 (m, 1H), 2.23 |
| | (d, J = 19.5 Hz, 3H), 2.16 (s, 3H). |
| 394 | N-(3-(3,3-difluoro-4-(methylamino)pyrrolidin-1-yl)-1-(2-(1,1- |
| | difluoroethyl)pyrimidin-4-yl)-1H-pyrazolo[4,3-c]pyridin-6-yl)acetamide |
| | 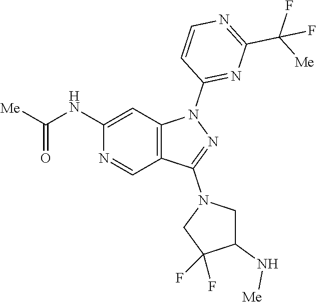 [SMILES: CNC1CN(c2nn(-c3ccnc(C(C)(F)F)n3)c3cc(NC(C)=O)ncc23)CC1(F)F] |
| | SM: 1-(6-chloro-1-(2-(1, 1-difluoroethyl)pyrimidin-4-yl)-1H-pyrazolo[4,3- |
| | c]pyridin-3-yl)-4,4-difluoro-N-methylpyrrolidin-3-amine (Preparation 662) |
| | Prep-HPLC-P (35% to 65% MeCN) 25 mg, 23.8% yield as a yellow solid. |
| | LCMS /z = 453.2 [M + H]⁺ ¹H NMR (400 MHz, CDCl₃) δ: ppm 9.51 (s, 1H), |
| | 8.77 (d, J = 5.6 Hz, 1H), 8.69 (s, 1H), 8.07 (s, 1H), 7.78 (d, J = 5.6 Hz, 1H), 4.08- |
| | 4.21 (m, 3H), 3.59-3.65 (m, 2H), 2.64 (s, 3H), 2.24-2.33 (m, 6H). |
| 395 | N-(1-(2-(1, 1-difluoroethyl)pyrimidin-4-yl)-3-(4-(dimethylamino)-3,3- |
| | difluoropyrrolidin-1-y1)-1H-pyrazolo[4,3-c]pyridin-6-yl)acetamide |
| | 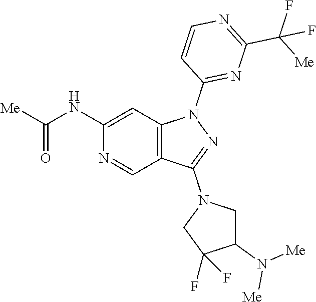 [SMILES: CC(=O)Nc1cc2c(cn1)c(N1CC(N(C)C)C(F)(F)C1)nn2-c1ccnc(C(C)(F)F)n1] |
| | SM: 1-(6-chloro-1-(2-(1, 1-difluoroethyl)pyrimidin-4-yl)-1H-pyrazolo[4,3- |
| | c]pyridin-3-y1)-4,4-difluoro-N,N-dimethylpyrrolidin-3-amine (Preparation 663) |
| | Prep-HPLC-P (30% to 60% MeCN). 28.2 mg, 26.8% yield as a yellow solid. |
| | LCMS m/z = 467.2 [M + H]⁺ ¹H NMR (400 MHz, CDCl₃) δ: ppm 9.52 (s, 1H), |
| | 8.77 (d, J = 5.6 Hz, 1H), 8.68 (s, 1H), 8.11 (s, 1H), 7.78 (d, J = 5.6 Hz, 1H), 4.11- |
| | 4.22 (m, 3H), 3.79-3.83 (m, 1H) 3.21-3.27 (m, 1H), 2.50 (s, 6H), 2.24-2.33 (m, |
| | 6H). |
| 396 | N-(3-(3,3-difluoro-4-methoxypyrrolidin-1-yl)-1-(2-(1,1- |
| | difluoroethyl)pyrimidin-4-yl)-1H-pyrazolo[4,3-c]pyridin-6-yl)acetamide |
| | 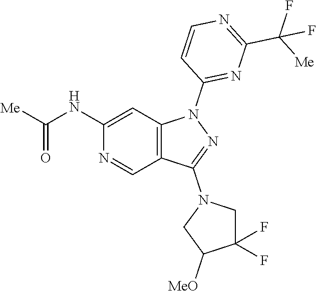 [SMILES: COC1CN(c2nn(-c3ccnc(C(C)(F)F)n3)c3cc(NC(C)=O)ncc23)CC1(F)F] |
| | SM: 6-chloro-3-(3,3-difluoro-4-methoxypyrrolidin-1-yl)-1-(2-(1,1- |
| | difluoroethyl)pyrimidin-4-y1)-1H-pyrazolo[4,3-c]pyridine (Preparation 654) |
| | Prep-HPLC-J (33% to 63% MeCN). 2.5 mg, 11.9% yield as a white solid. |
| | LCMS m/z = 454.1 [M + H]⁺. ¹H NMR (400 MHz, CDCl₃) δ ppm: 9.52 (s, 1H), |
| | 8.68-8.77 (m, 2H), 8.09 (s, 1H), 7.77 (d, J = 5.6 Hz, 1H), 4.05-4.16 (m, 4H), |
| | 3.89-3.93 (m, 1H), 3.62 (s, 3H), 2.24-2.33 (m, 6H). |
| 397 | (S)-N-(1-(2-(1,1-difluoroethyl)-6-ethoxypyrimidin-4-yl)-3-(3- |
| | (dimethylamino)pyrrolidin-1-yl)-1H-pyrazolo[4,3-c]pyridin-6-yl)acetamide |
| | 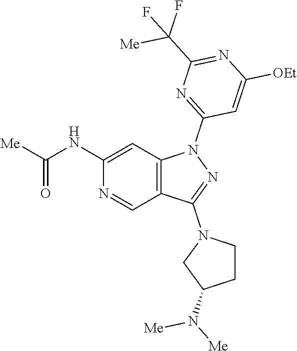 [SMILES: CCOc1cc(-n2nc(N3CC[C@H](N(C)C)C3)c3cnc(NC(C)=O)cc32)nc(C(C)(F)F)n1] |
| | SM: (S)-1-(6-chloro-1-(2-(1,1-difluoroethyl)-6-ethoxypyrimidin-4-yl)-1H- |
| | pyrazolo[4,3-c]pyridin-3-yl)-N,N-dimethylpyrrolidin-3-amine (Preparation |
| | 689) |
| | Prep-HPLC-C (43% to 73% MeCN) 42 mg, 50% as white solid. LCMS m/z = |
| | 475.2 [M + H]⁺ ¹H NMR (500 MHz, CDCl₃) δ ppm: 9.45 (s, 1H), 8.70 (s, 1H), |
| | 7.99 (s, 1H), 7.11 (s, 1H), 4.50-4.54 (m, 2H), 3.94 (q, J = 7.0 Hz, 2H), 3.75-3.77 |
| | (m, 2H), 3.71-3.73 (m, 1H), 3.55 (br s, 1H), 2.94 (br s, 1H), 2.20-2.43 (m, |
| | 13H), 1.99-2.03 (m, 1H), 1.43 (t, J = 7.5 Hz, 3H) |
| 398 | N-(1-(2-(1,1-difluoroethyl)-6-methylpyrimidin-4-y1)-3-(3-(dimethylamino)-3- |
| | methylpyrrolidin-1-yl)-1H-pyrazolo[4,3-c]pyridin-6-yl)acetamide |
| | 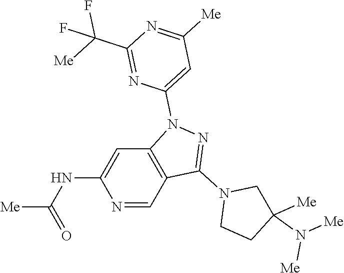 [SMILES: CC(=O)Nc1cc2c(cn1)c(N1CCC(C)(N(C)C)C1)nn2-c1cc(C)nc(C(C)(F)F)n1] |
| | SM: 1-(6-chloro-1-(2-(1, 1-difluoroethyl)-6-methylpyrimidin-4-y1)-1H- |
| | pyrazolo[4,3-c]pyridin-3-y1)-N,N,3-trimethylpyrrolidin-3-amine (Preparation |
| | 701) |
| | Prep-HPLC-C (35% to 65% MeCN) 14.8 mg, 23.5% as white solid. LCMS |
| | m/z = 459.2 [M + H]⁺ ¹H NMR: (400 MHz, CDCl₃) δ ppm: 9.47 (s, 1H), 8.71 (s, |
| | 1H), 8.06 (s, 1H), 7.64 (s, 1H), 3.93-3.95 (m, 1H), 3.81-3.86 (m, 1H), 3.68 (s, |
| | 2H), 2.64 (s, 3H), 2.38 (s, 6H), 2.24-2.33 (m, 6H), 2.16-2.17 (m, 1H), 2.01-2.04 |
| | (m, 1H), 1.18 (s, 3H). |
| 399 | N-(1-(2-(1,1-difluoroethyl)-6-methylpyrimidin-4-yl)-3-(3-(difluoromethyl)-3- |
| | hydroxypyrrolidin-1-yl)-1H-pyrazolo[4,3-c]pyridin-6-yl)acetamide |
| | 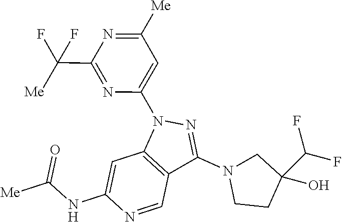 [SMILES: CC(=O)Nc1cc2c(cn1)c(N1CCC(O)(C(F)F)C1)nn2-c1cc(C)nc(C(C)(F)F)n1] |
| | SM: 1-(6-chloro-1-(2-(1, 1-difluoroethyl)-6-methylpyrimidin-4-y1)-1H- |
| | pyrazolo[4,3-c]pyridin-3-yl)-3-(difluoromethyl)pyrrolidin-3-ol (Preparation |
| | 668) |
| | Prep-HPLC-C (32% to 60% MeCN) 42.6 mg, 45% as white solid. LCMS m/z = |
| | 468.1 [M + H]⁺ ¹H NMR (400 MHz, MeOD-d₄) δ ppm: 9.16 (s, 1H), 8.69 (s, |
| | 1H), 7.52 (s, 1H), 5.99 (t, J = 55.6 Hz, 1H), 3.82-3.90 (m, 3H), 3.66-3.69 (m, |
| | 1H), 2.56 (s, 3H), 2.13-2.56 (m, 8H). |
| 400 | N-(1-(2-(1,1-difluoroethyl)-6-methylpyrimidin-4-yl)-3-(methyl(1- |
| | methylpyrrolidin-3-yl)amino)-1H-pyrazolo[4,3-c]pyridin-6-yl)acetamide |
| | 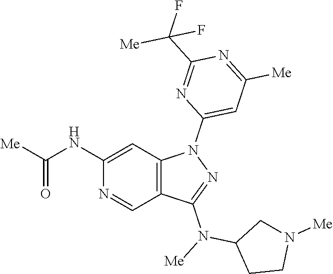 [SMILES: CC(=O)Nc1cc2c(cn1)c(N(C)C1CCN(C)C1)nn2-c1cc(C)nc(C(C)(F)F)n1] |
| | SM: 6-chloro-1-(2-(1,1-difluoroethyl)-6-methylpyrimidin-4-yl)-N-methyl-N- |
| | (1-methylpyrrolidin-3-y1)-1H-pyrazolo[4,3-c]pyridin-3-amine (Preparation |
| | 674) |
| | Prep-HPLC-C (33% to 60% MeCN) 35.2 mg, 47.7% as white solid. LCMS |
| | m/z = 445.1 [M + H]⁺ ¹H NMR: (400 MHz, CDCl₃) δ ppm: 9.49 (s, 1H), 8.78 (s, |
| | 1H), 8.07 (s, 1H), 7.63 (s, 1H), 4.95-5.02 (m, 1H), 3.27 (s, 3H), 2.77-2.82 (m, |
| | 3H), 2.75 (s, 3H), 2.63-2.65 (m, 1H), 2.40 (s, 3H), 2.24-2.33 (m, 7H), 1.99-2.00 |
| | (m, 1H). |
| 401 | N-(1-(2-(1,1-difluoroethyl)-6-methylpyrimidin-4-yl)-3-(3-(dimethylamino)-3- |
| | methylazetidin-1-y1)-1H-pyrazolo[4,3-c]pyridin-6-yl)acetamide |
| | 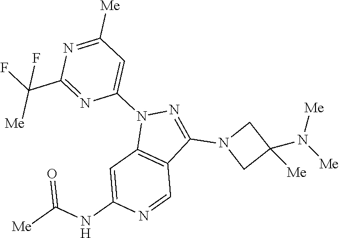 [SMILES: CC(=O)Nc1cc2c(cn1)c(N1CC(C)(N(C)C)C1)nn2-c1cc(C)nc(C(C)(F)F)n1] |
| | SM: 1-(6-chloro-1-(2-(1, 1-difluoroethyl)-6-methylpyrimidin-4-y1)-1H- |
| | pyrazolo[4,3-c]pyridin-3-yl)-N,N,3-trimethylazetidin-3-amine (Preparation |
| | 666) |
| | Prep-HPLC-C (28% to 58% MeCN). 25.8 mg, 25.5% as white solid. LCMS |
| | m/z = 445.2 [M + H]⁺ ¹H NMR (400 MHz, CDCl₃) δ ppm: 9.43 (s, 1H), 8.52 (s, |
| | 1H), 8.13 (s, 1H), 7.62 (s, 1H), 4.21 (d, J = 7.2 Hz, 2H), 3.98 (d, J = 7.2 Hz, 2H), |
| | 2.63 (s, 3H), 2.23-2.33 (m, 12H), 1.46 (s, 3H). |

### Example 402: N-(1-(2-(1,1-difluoroethyl)-6-methylpyrimidin-4-yl)-3-(6-ethyl-3,6-diazabicyclo[3.1.1]heptan-3-yl)-1H-pyrazolo[4,3-c]pyridin-6-yl)propionamide

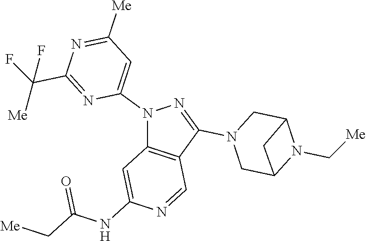 [SMILES: CCC(=O)Nc1cc2c(cn1)c(N1CC3CC(C1)N3CC)nn2-c1cc(C)nc(C(C)(F)F)n1]

N-(1-(2-(1,1-Difluoroethyl)-6-methylpyrimidin-4-yl)-3-(6-ethyl-3,6-diazabicyclo[3.1.1]heptan-3-yl)-1H-pyrazolo[4,3-c]pyridin-6-yl)propionamide was obtained as a white solid, 26 mg, 34.3%, from 3-(6-chloro-1-(2-(1,1-difluoroethyl)-6-methylpyrimidin-4-yl)-1H-pyrazolo[4,3-c]pyridin-3-yl)-6-ethyl-3,6-diazabicyclo[3.1.1]heptane (Preparation 693) and propionamide, following a similar procedure to that described in Example 390. LCMS m/z=471.2 [M+H]⁺. ¹H NMR (400 MHz, CDCl₃) δ ppm: 9.53 (s, 1H), 8.84 (s, 1H), 8.02 (s, 1H), 7.66 (s, 1H), 4.05 (d, J=10.8 Hz, 2H), 3.91 (d, J=5.6 Hz, 2H), 3.79 (d, J=11.2 Hz, 2H), 2.80 (br s, 1H), 2.65 (s, 3H), 2.59-2.61 (m, 2H), 2.47-2.50 (m, 2H), 2.31 (t, J=19.2 Hz, 3H), 1.78-1.80 (m, 1H), 1.31 (t, J=7.6 Hz, 3H), 1.10 (t, J=7.2 Hz, 3H).

### Example 403: N-(1-(2-(1,1-difluoroethyl)-6-methylpyrimidin-4-yl)-3-(3-(dimethylamino)-3-methylpyrrolidin-1-yl)-1H-pyrazolo[4,3-c]pyridin-6-yl)acetamide

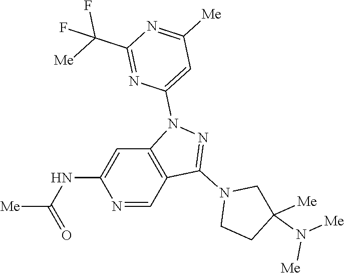 [SMILES: CC(=O)Nc1cc2c(cn1)c(N1CCC(C)(N(C)C)C1)nn2-c1cc(C)nc(C(C)(F)F)n1]

To a solution of 1-(6-chloro-1-(2-(1,1-difluoroethyl)-6-methylpyrimidin-4-yl)-1H-pyrazolo[4,3-c]pyridin-3-yl)-N,N,3-trimethylpyrrolidin-3-amine (Preparation 701, 100 mg, 0.229 mmol) in dioxane (5 mL) was added methyl carbamate (43.1 mg, 0.574 mmol), Pd₂(dba)₃ (21.0 mg, 0.023 mmol), Xantphos (26.6 mg, 0.046 mmol) and K₃PO₄ (146.1 mg, 0.688 mmol). The reaction was stirred at 100° C. for 16 h under N₂. The mixture was concentrated and purified by prep-HPLC-C (45% to 65% MeCN) to give N-(1-(2-(1,1-difluoroethyl)-6-methylpyrimidin-4-yl)-3-(3-(dimethylamino)-3-methylpyrrolidin-1-yl)-1H-pyrazolo[4,3-c]pyridin-6-yl)acetamide (13.2 mg, 12.1% yield) as a white solid. LCMS m/z=475.2 [M+H]^{+ 1}H NMR (500 MHz, CDCl₃) δ ppm: 9.21 (s, 1H), 8.71 (s, 1H), 7.63-7.65 (m, 2H), 3.96-3.99 (m, 1H), 3.79-3.91 (m, 5H), 3.72 (d, J=9.5 Hz, 1H), 2.64 (s, 3H), 2.31-2.63 (m, 7H), 2.26 (t, J=18.5 Hz, 3H), 2.04-2.12 (m, 1H), 1.25-1.28 (m, 3H).

### Example 404: N-(1-(6-(1,1-difluoroethyl)pyrazin-2-yl)-3-(3-(dimethylamino)-3-methylpyrrolidin-1-yl)-1H-pyrazolo[4,3-c]pyridin-6-yl)acetamide

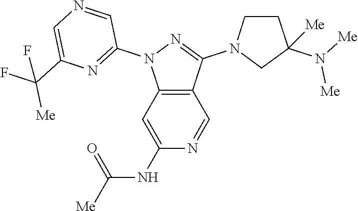 [SMILES: CC(=O)Nc1cc2c(cn1)c(N1CCC(C)(N(C)C)C1)nn2-c1cncc(C(C)(F)F)n1]

N-(1-(6-(1,1-Difluoroethyl)pyrazin-2-yl)-3-(3-(dimethylamino)-3-methylpyrrolidin-1-yl)-1H-pyrazolo[4,3-c]pyridin-6-yl)acetamide was obtained as white solid, 23.3 mg, 14.7% from 1-(6-chloro-1-(6-(1,1-difluoroethyl)pyrazin-2-yl)-1H-pyrazolo[4,3-c]pyridin-3-yl)-N,N,3-trimethylpyrrolidin-3-amine (Preparation 702) and acetamide, following the procedure described in Example 403. LCMS m/z=445.2 [M+H]⁺. ¹H NMR: (400 MHz, CDCl₃) δ ppm: 9.33 (s, 1H), 9.28 (s, 1H), 8.73 (s, 1H), 8.66 (s, 1H), 8.06 (s, 1H), 3.69-3.96 (m, 4H), 2.26-2.40 (m, 12H), 1.82-2.16 (m, 2H), 1.21 (s, 3H).

### Example 405: N-(1-(2-(1,1-difluoroethyl)pyrimidin-4-yl)-3-(9-methyl-3,9-diazabicyclo[3.3.1]nonan-3-yl)-1H-pyrazolo[4,3-c]pyridin-6-yl)acetamide

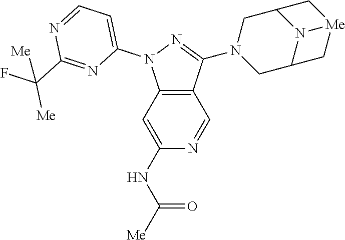 [SMILES: CC(=O)Nc1cc2c(cn1)c(N1CC(C)N(C)C(C)C1)nn2-c1ccnc(C(C)(C)F)n1]

To a solution of 6-chloro-1-(2-(1,1-difluoroethyl)pyrimidin-4-yl)-3-(9-methyl-3,9-diazabicyclo[3.3.1]nonan-3-yl)-1H-pyrazolo[4,3-c]pyridine (Preparation 650) (30 mg, 0.069 mmol) in dioxane (2 mL) was added acetamide (8.2 mg, 0.138 mmol), BrettPhos Pd G₃ (6.3 mg, 6.91 μmol) and Cs₂CO₃ (45.1 mg, 0.138 mmol) and the reaction was stirred at 100° C. for 5 h under N₂. The mixture was concentrated and was purified by Prep-HPLC-P (31% to 60% MeCN) to give N-(1-(2-(1,1-difluoroethyl)pyrimidin-4-yl)-3-(9-methyl-3,9-diazabicyclo[3.3.1]nonan-3-yl)-1H-pyrazolo[4,3-c]pyridin-6-yl)acetamide (2.6 mg, 8.2% yield) as a yellow solid. LCMS m/z=457.2 [M+H]^{+ 1}H NMR: (400 MHz, CDCl₃) δ: ppm 9.54 (s, 1H), 8.83 (s, 1H), 8.76 (d, J=6.0 Hz, 1H), 8.09 (s, 1H), 7.80 (d, J=5.6 Hz, 1H), 3.92-3.95 (m, 2H), 3.78-3.81 (m, 2H), 3.06 (br s, 2H), 2.69 (s, 3H), 2.23-2.34 (m, 8H), 1.72-1.74 (m, 4H).

Examples 406 to 434: The compounds in the following table were prepared from the appropriate 6-chloro-1H-pyrazolo[4,3-c]pyridine and acetamide, following a similar procedure to that described in Example 405

| Example | |
|---|---|
| No | Name, Structure, Starting Materials (SM), Purification, Data |
| 406 | N-(1-(2-(1,1-difluoroethyl)pyrimidin-4-yl)-3-(4-hydroxy-2-methylpyrrolidin- |
| | 1-y1)-1H-pyrazolo[4,3-c]pyridin-6-yl)acetamide |
| | 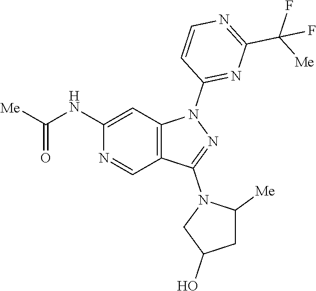 [SMILES: CC(=O)Nc1cc2c(cn1)c(N1CC(O)CC1C)nn2-c1ccnc(C(C)(F)F)n1] |
| | SM: 1-(6-chloro-1-(2-(1, 1-difluoroethyl)pyrimidin-4-yl)-1H-pyrazolo[4,3- |
| | c]pyridin-3-yl)-5-methylpyrrolidin-3-ol (Preparation 653) |
| | prep-HPLC-F (35% to 65% MeCN) 4.5 mg, 42.6% yield as a white solid. |
| | LCMS m/z = 418.0 [M + H]⁺. ¹H NMR (400 MHz, CDCl₃) δ ppm: 9.51 (s, |
| | 1H), 8.71-8.75 (m, 2H), 8.07 (s, 1H), 7.78 (d, J = 5.6 Hz, 1H), 4.70-4.72 (m, |
| | 1H), 4.37-4.41 (m, 1H), 3.88-3.99 (m, 2H), 2.42-2.48 (m, 1H), 2.24-2.34 (m, |
| | 6H), 1.91-1.95 (m, 1H), 1.82-1.84 (m, 1H), 1.58 (s, 3H). |
| 407 | N-(1-(2-(1,1-difluoroethyl)pyrimidin-4-yl)-3-(4-methoxy-2-methylpyrrolidin- |
| | 1-y1)-1H-pyrazolo[4,3-c]pyridin-6-yl)acetamide |
| | 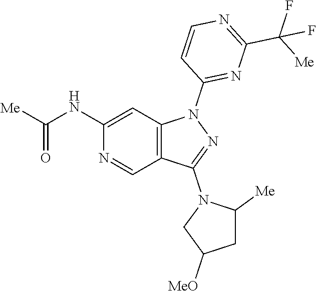 [SMILES: COC1CC(C)N(c2nn(-c3ccnc(C(C)(F)F)n3)c3cc(NC(C)=O)ncc23)C1] |
| | SM: 6-chloro-1-(2-(1, 1-difluoroethyl)pyrimidin-4-yl)-3-(4-methoxy-2- |
| | methylpyrrolidin-1-yl)-1H-pyrazolo[4,3-c]pyridine (Preparation 655) |
| | Prep TLC 100% EtOAc. 10.0 mg, 47.4% yield as a yellow solid. LCMS |
| | m/z = 432.1 [M + H]⁺. ¹H NMR (500 MHz, CDCl₃) δ ppm: 9.50 (s, 1H), 8.74 (d, |
| | J = 6.0 Hz, 1H), 8.71 (s, 1H), 8.12 (s, 1H), 7.79 (d, J = 6.0 Hz, 1H), 4.36-4.42 |
| | (m, 1H), 4.17-4.19 (m, 1H), 4.01-4.06 (m, 1H), 3.91-3.85 (m, 1H), 3.38 (s, |
| | 3H), 2.36-2.39 (m, 1H), 2.30 (t, J = 18.4 Hz, 3H), 2.29 (s, 3H), 1.90-1.94 (m, |
| | 1H), 1.43 (d, J = 6.0 Hz, 3H) |
| 408 | (R)-N-(1-(6-(1,1-difluoroethyl)pyridin-2-yl)-3-(3-(dimethylamino)pyrrolidin- |
| | 1-y1)-1H-pyrazolo[4,3-c]pyridin-6-yl)acetamide |
| | 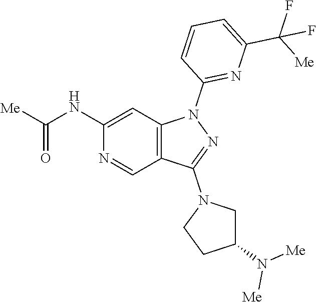 [SMILES: CC(=O)Nc1cc2c(cn1)c(N1CC[C@@H](N(C)C)C1)nn2-c1cccc(C(C)(F)F)n1] |
| | SM: (R)-1-(6-chloro-1-(6-(1,1-difluoroethyl)pyridin-2-y1)-1H-pyrazolo[4,3- |
| | c]pyridin-3-y1)-N,N-dimethylpyrrolidin-3-amine (Preparation 690) |
| | Prep-HPLC-P (34% to 64% MeCN) 55 mg, 21.7% yield as a yellow solid. |
| | LCMS m/z = 430.2 [M + H]⁺. ¹H NMR (400 MHz, CDCl₃) δ: ppm 9.38 (s, |
| | 1H), 8.72 (s, 1H), 7.99 (s, 1H), 7.95 (d, J = 8.4 Hz, 1H), 7.84-7.88 (m, 1H), |
| | 7.43 (d, J = 7.6 Hz, 1H) 3.93-3.99 (m, 2H), 3.73-3.79 (m, 1H), 3.61 (br s, 1H), |
| | 3.06 (br s, 1H), 2.26-2.43 (m, 13H), 2.12 (br s, 1H). |
| 409 | N-(1-(2-(1,1-difluoroethyl)pyrimidin-4-y1)-3-(3-hydroxy-4-methylpyrrolidin- |
| | 1-yl)-1H-pyrazolo[4,3-c]pyridin-6-yl)acetamide |
| | 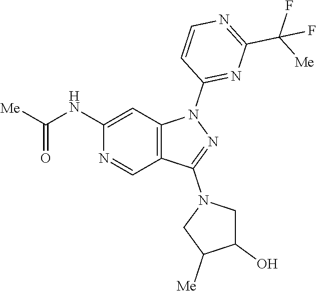 [SMILES: CC(=O)Nc1cc2c(cn1)c(N1CC(C)C(O)C1)nn2-c1ccnc(C(C)(F)F)n1] |
| | SM: 1-(6-chloro-1-(2-(1, 1-difluoroethyl)pyrimidin-4-yl)-1H-pyrazolo[4,3- |
| | c]pyridin-3-yl)-4-methylpyrrolidin-3-ol (Preparation 692) |
| | prep-HPLC-I (30% to 50% MeCN) 10 mg, 14.6% yield as a yellow solid. |
| | LCMS m/z = 418.1 [M + H]⁺. ¹H NMR (500 MHz, MeOD-d₄) δ ppm: 9.03-9.04 |
| | (m, 1H), 8.93-8.95 (m, 1H), 8.67-8.69 (m, 1H), 8.02-8.06 (m, 1H), 4.38-4.41 |
| | (m, 0.2H), 4.19-4.21 (m, 0.8H), 4.06-4.09 (m, 1.6H), 3.93-4.01 (m, 0.4H), |
| | 3.73-3.76 (m, 0.2H), 3.64-3.67 (m, 0.8H), 3.49-3.53 (m, 1H), 2.42-2.46 (m, |
| | 1H), 2.38 (s, 3H), 2.18 (t, J = 19.0 Hz, 3H), 1.17-1.27 (m, 3H). |
| 410 | N-(1-(2-(1,1-difluoroethyl)pyrimidin-4-y1)-3-(3-methoxy-4-methylpyrrolidin- |
| | 1-y1)-1H-pyrazolo[4,3-c]pyridin-6-yl)acetamide |
| | 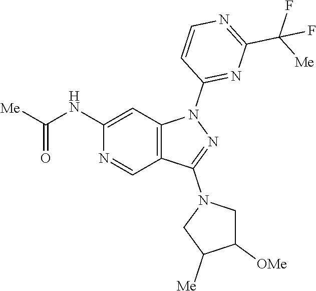 [SMILES: COC1CN(c2nn(-c3ccnc(C(C)(F)F)n3)c3cc(NC(C)=O)ncc23)CC1C] |
| | SM: 6-chloro-1-(2-(1, 1-difluoroethyl)pyrimidin-4-yl)-3-(3-methoxy-4- |
| | methylpyrrolidin-1-y1)-1H-pyrazolo[4,3-c]pyridine (Preparation 656) |
| | prep-HPLC-I (37% to 57% MeCN). 7.0 mg, 11.1% yield as a white solid. |
| | LCMS m/z = 432.1 [M + H]⁺. ¹H NMR (500 MHz, CDCl₃) δ ppm: 9.66 (s, |
| | 1H), 9.45 (s, 1H), 8.80 (d, J = 5.5 Hz, 1H), 8.73 (s, 1H), 7.83 (d, J = 5.5 Hz, |
| | 1H), 3.93-3.99 (m, 2H), 3.72-3.75 (m, 2H), 3.43-3.45 (m, 4H), 2.57-2.58 (m, |
| | 1H), 2.32 (s, 3H), 2.27 (t, J = 19.0 Hz, 3H), 1.15 (d, J = 7.0 Hz, 3H) |
| 411 | N-(1-(2-(1,1-difluoroethyl)pyrimidin-4-yl)-3-(3-(dimethylamino)-4- |
| | methylpyrrolidin-1-yl)-1H-pyrazolo[4,3-c]pyridin-6-yl)acetamide |
| | 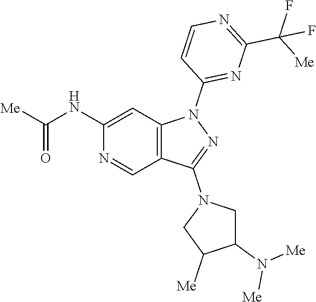 [SMILES: CC(=O)Nc1cc2c(cn1)c(N1CC(C)C(N(C)C)C1)nn2-c1ccnc(C(C)(F)F)n1] |
| | SM: 1-(6-chloro-1-(2-(1, 1-difluoroethyl)pyrimidin-4-yl)-1H-pyrazolo[4,3- |
| | c]pyridin-3-yl)-N,N,4-trimethylpyrrolidin-3-amine (Preparation 658) |
| | prep-HPLC-F (41% to 71% MeCN) 8.0 mg, 30.4% yield as white solid. |
| | LCMS m/z = 445.3 [M + H]⁺ ¹H NMR (500 MHz, CDCl₃) δ ppm: 9.50 (s, 1H), |
| | 8.74 (s, 1H), 8.73 (s, 1H), 8.12 (s, 1H), 7.71-7.80 (m, 1H),3.36-4.02 (m, 4H), |
| | 2.72-3.01 (m, 1H), 2.54-2.56 (m, 1H), 2.45 (s, 3H), 2.34 (s, 3H), 2.23-2.29 |
| | (m, 6H), 1.12-1.25 (m, 3H). |
| 412 | (S)-N-(1-(2-(1,1-difluoroethyl)-6-methoxypyrimidin-4-yl)-3-(3- |
| | (dimethylamino)pyrrolidin-1-yl)-1H-pyrazolo[4,3-c]pyridin-6-yl)acetamide |
| | 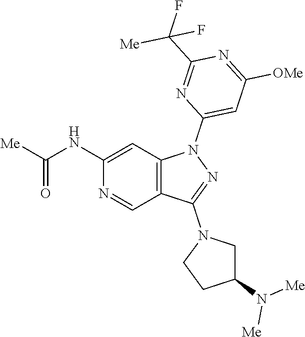 [SMILES: COc1cc(-n2nc(N3CC[C@H](N(C)C)C3)c3cnc(NC(C)=O)cc32)nc(C(C)(F)F)n1] |
| | SM: (S)-1-(6-chloro-1-(2-(1,1-difluoroethyl)-6-methoxypyrimidin-4-yl)-1H- |
| | pyrazolo[4,3-c]pyridin-3-yl)-N,N-dimethylpyrrolidin-3-amine (Preparation |
| | 698) |
| | Prep-HPLC-C (38% to 67% MeCN). 95.0 mg, 75.3% yield as a white solid. |
| | LCMS m/z = 461.1 [M + H]⁺ ¹H NMR (500 MHz, CDCl₃) δ ppm : 9.44 (s, |
| | 1H), 8.70 (s, 1H), 8.03 (s, 1H), 7.12 (s, 1H), 4.08 (s, 3H), 3.90-3.96 (m, 2H), |
| | 3.73-3.76 (m, 1H), 3.55-3.57 (m, 1H), 2.94-3.02 (m, 1H), 2.39 (s, 6H), 2.21- |
| | 2.31 (m, 7H), 2.06-2.09 (m, 1H). |
| 413 | (S)-N-(1-(2-(1,1-difluoroethyl)-6-isopropoxypyrimidin-4-yl)-3-(3- |
| | (dimethylamino)pyrrolidin-1-yl)-1H-pyrazolo[4,3-c]pyridin-6-yl)acetamide |
| | 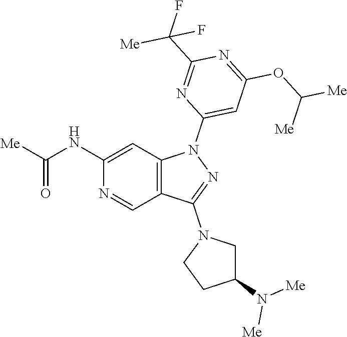 [SMILES: CC(=O)Nc1cc2c(cn1)c(N1CC[C@H](N(C)C)C1)nn2-c1cc(OC(C)C)nc(C(C)(F)F)n1] |
| | SM: (S)-1-(6-chloro-1-(2-(1,1-difluoroethyl)-6-isopropoxypyrimidin-4-yl)- |
| | 1H-pyrazolo[4,3-c]pyridin-3-y1)-N,N-dimethylpyrrolidin-3-amine |
| | (Preparation 682) |
| | Prep-HPLC-C (45% to 75% MeCN) 69 mg, 43.9% yield as a white solid. |
| | LCMS m/z = 489.2 [M + H]⁺ ¹H NMR (500 MHz, CDCl₃) δ ppm: 9.45 (s, 1H), |
| | 8.70 (s, 1H), 8.03 (s, 1H), 7.08 (s, 1H), 5.45-5.55 (m, 1H), 3.89-3.97 (m, 2H), |
| | 3.69-3.77 (m, 1H), 3.50-3.60 (m, 1H), 2.98 (br s, 1H), 2.40 (s, 6H), 2.19-2.31 |
| | (m, 7H), 2.04-2.12 (m, 1H), 1.39 (d, J = 6.5 Hz, 6H). |
| 414 | (S)-N-(1-(2-(1,1-difluoroethyl)-6-ethylpyrimidin-4-y1)-3-(3- |
| | (dimethylamino)pyrrolidin-1-yl)-1H-pyrazolo[4,3-c]pyridin-6-yl)acetamide |
| | 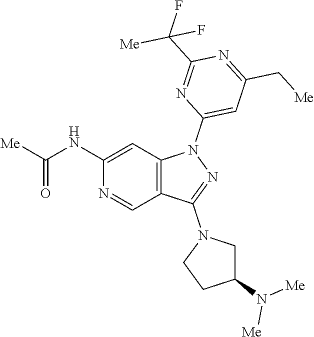 [SMILES: CCc1cc(-n2nc(N3CC[C@H](N(C)C)C3)c3cnc(NC(C)=O)cc32)nc(C(C)(F)F)n1] |
| | SM: (S)-1-(6-chloro-1-(2-(1,1-difluoroethyl)-6-ethylpyrimidin-4-yl)-1H- |
| | pyrazolo[4,3-c]pyridin-3-yl)-N,N-dimethylpyrrolidin-3-amine (Preparation |
| | 683) |
| | Prep-HPLC-C (37% to 65% MeCN) 50.6 mg, 53.5% yield as a white solid. |
| | LCMS m/z = 459.2 [M + H]⁺ ¹H NMR (400 MHz, CDCl₃) δ ppm: 9.48 (s, 1H), |
| | 8.73 (s, 1H), 8.02 (s, 1H), 7.63 (s, 1H), 3.93-3.99 (m, 2H), 3.75-3.77 (m, 1H), |
| | 3.56-3.58 (m, 1H), 2.88-2.96 (m, 3H), 2.38 (s, 6H), 2.27-2.31 (m, 7H), 2.05- |
| | 2.24 (m, 1H), 1.38 (t, J = 7.6 Hz, 3H). |
| 415 | (S)-N-(1-(6-cyclopropyl-2-(1,1-difluoroethyl)pyrimidin-4-yl)-3-(3- |
| | (dimethylamino)pyrrolidin-1-y1)-1H-pyrazolo[4,3-c]pyridin-6-yl)acetamide |
| | 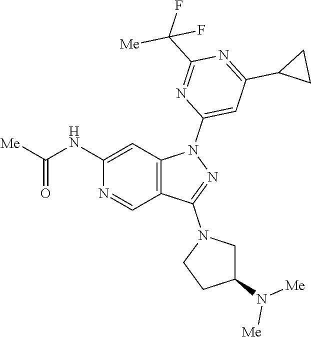 [SMILES: CC(=O)Nc1cc2c(cn1)c(N1CC[C@H](N(C)C)C1)nn2-c1cc(C2CC2)nc(C(C)(F)F)n1] |
| | SM: (S)-1-(6-chloro-1-(6-cyclopropyl-2-(1,1-difluoroethyl)pyrimidin-4-yl)- |
| | 1H-pyrazolo[4,3-c]pyridin-3-yl)-N,N-dimethylpyrrolidin-3-amine |
| | (Preparation 684) |
| | prep-HPLC-Q (15% to 35% MeCN) 55.8 mg, 35.4% yield as a light-yellow |
| | solid. LCMS m/z = 471.2 [M + H]⁺. ¹H NMR: (400 MHz, CDCl₃) δ ppm: 9.47 |
| | (s, 1H), 8.66 (s, 1H), 8.14 (s, 1H), 7.56 (s, 1H), 3.96-4.01 (m, 2H), 3.73-3.76 |
| | (m, 2H), 3.30-3.41 (m, 1H), 2.61 (s, 6H), 2.35-2.45 (m, 1H), 2.11-2.26 (m, |
| | 8H), 1.23-1.28 (m, 2H), 1.09-1.13 (m, 2H). |
| 416 | (S)-N-(1-(2-(1,1-difluoroethyl)-6-(2-methoxyethoxy)pyrimidin-4-yl)-3-(3- |
| | (dimethylamino)pyrrolidin-1-yl)-1H-pyrazolo[4,3-c]pyridin-6-yl)acetamide |
| | 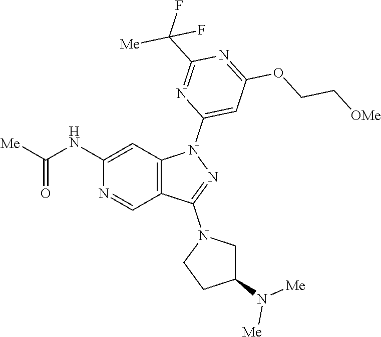 [SMILES: COCCOc1cc(-n2nc(N3CC[C@H](N(C)C)C3)c3cnc(NC(C)=O)cc32)nc(C(C)(F)F)n1] |
| | SM: (S)-1-(6-chloro-1-(2-(1,1-difluoroethyl)-6-(2-methoxyethoxy)pyrimidin- |
| | 4-y1)-1H-pyrazolo[4,3-c]pyridin-3-yl)-N,N-dimethylpyrrolidin-3-amine |
| | (Preparation 685) |
| | prep-HPLC-P (37% to 67% MeCN) 69 mg, 31.4% yield as a white solid. |
| | LCMS m/z = 505.1 [M + H]⁺ ¹H NMR (400 MHz, CDCl₃) δ: ppm 9.46 (s, 1H), |
| | 8.72 (s, 1H), 8.03 (s, 1H), 7.26 (s, 1H), 4.65-4.68 (m, 2H), 3.94-3.98 (m, 2H), |
| | 3.79-3.81 (m, 2H), 3.75-3.77 (m, 1H), 3.52-3.55 (m, 1H), 3.47 (s, 3H), 2.94- |
| | 2.97 (m, 1H), 2.39 (s, 6H), 2.22-2.29 (m, 7H), 2.06-2.09 (m, 1H). |
| 417 | (S)-N-(1-(6-cyclopropoxy-2-(1,1-difluoroethyl)pyrimidin-4-y1)-3-(3- |
| | (dimethylamino)pyrrolidin-1-yl)-1H-pyrazolo[4,3-c]pyridin-6-yl)acetamide |
| | 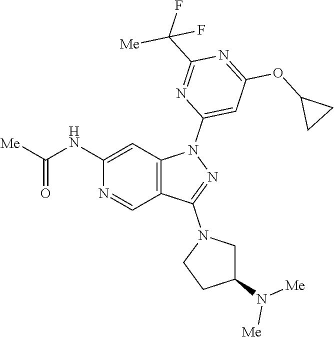 [SMILES: CC(=O)Nc1cc2c(cn1)c(N1CC[C@H](N(C)C)C1)nn2-c1cc(OC2CC2)nc(C(C)(F)F)n1] |
| | SM: (S)-1-(6-chloro-1-(6-cyclopropoxy-2-(1,1-difluoroethyl)pyrimidin-4-yl)- |
| | 1H-pyrazolo[4,3-c]pyridin-3-yl)-N,N-dimethylpyrrolidin-3-amine |
| | (Preparation 687) |
| | Prep-HPLC-C (37% to 66% MeCN) 7.5 mg, 14.3% as yellow solid. LCMS |
| | m/z = 487.2 [M + H]⁺ ¹HNMR (500 MHz, CDCl₃) δ: ppm 9.47 (s, 1H), 8.70 (s, |
| | 1H), 8.10 (s, 1H), 7.21 (s, 1H), 4.39 (s, 1H), 3.90-3.95 (m, 2H), 3.72-3.76 (m, |
| | 1H), 3.52-3.55 (m, 1H), 2.96 (br s, 1H), 2.37 (s, 6H), 2.24-2.29 (m, 6H), 2.02- |
| | 2.06 (m, 2H), 0.86-0.87 (m, 4H). |
| 418 | N-(1-(2-(1,1-difluoroethyl)-6-((1r,3S)-3-methoxycyclobutoxy)pyrimidin-4- |
| | y1)-3-((S)-3-(dimethylamino)pyrrolidin-1-yl)-1H-pyrazolo[4,3-c]pyridin-6- |
| | yl)acetamide |
| | 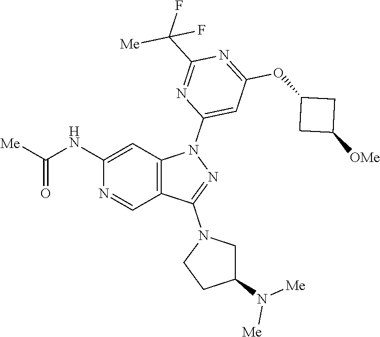 [SMILES: CO[C@H]1C[C@H](Oc2cc(-n3nc(N4CC[C@H](N(C)C)C4)c4cnc(NC(C)=O)cc43)nc(C(C)(F)F)n2)C1] |
| | SM: (S)-1-(6-chloro-1-(2-(1,1-difluoroethyl)-6-((1r,3S)-3- |
| | methoxycyclobutoxy)pyrimidin-4-yl)-1H-pyrazolo[4,3-c]pyridin-3-yl)-N,N- |
| | dimethylpyrrolidin-3-amine (Preparation 686) |
| | prep-HPLC-C (43% to 73% MeCN) 11.5 mg, 22.0% yield as a white solid. |
| | LCMS m/z = 530.2 [M + H]⁺ ¹H NMR (500 MHz, CDCl₃) δ ppm: 9.35 (s, 1H), |
| | 8.68 (s, 1H), 8.10 (s, 1H), 7.29 (s, 1H), 6.87 (s, 1H), 5.00-5.04 (m, 1H), 4.14- |
| | 4.17 (m, 1H), 3.93-3.96 (m, 2H), 3.91-3.92 (m, 1H), 3.73-3.75 (m, 1H), 3.29 |
| | (s, 3H), 3.01 (br s, 1H), 2.50-2.52 (m, 4H), 2.48-2.49 (m, 6H), 2.24-2.41 (m, |
| | 7H), 2.06-2.09 (m, 1H). |
| 419 | (S)-N-(1-(6-cyclobutoxy-2-(1,1-difluoroethyl)pyrimidin-4-y1)-3-(3- |
| | (dimethylamino)pyrrolidin-1-yl)-1H-pyrazolo[4,3-c]pyridin-6-yl)acetamide |
| | 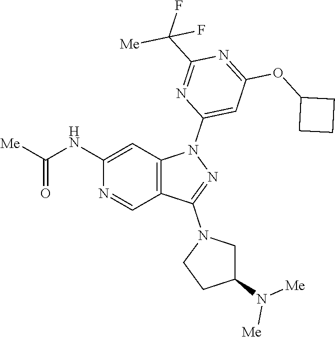 [SMILES: CC(=O)Nc1cc2c(cn1)c(N1CC[C@H](N(C)C)C1)nn2-c1cc(OC2CCC2)nc(C(C)(F)F)n1] |
| | SM: (S)-1-(6-chloro-1-(6-cyclobutoxy-2-(1,1-difluoroethyl)pyrimidin-4-yl)- |
| | 1H-pyrazolo[4,3-c]pyridin-3-yl)-N,N-dimethylpyrrolidin-3-amine |
| | (Preparation 699) |
| | prep-HPLC-F (63% to 93% MeCN) 28.1 mg, 26.8% yield as a white solid. |
| | LCMS m/z = 501.2 [M + H]⁺ ¹H NMR: (400 MHz, CDCl₃) δ ppm : 9.45 (s, |
| | 1H), 8.70 (s, 1H), 7.99 (s, 1H), 7.08 (s, 1H), 5.32-5.36 (m, 1H), 3.91-3.97 (m, |
| | 2H), 3.71-3.78 (m, 1H), 3.51-3.66 (m, 1H), 2.90-3.11 (m, 1H), 2.49-2.51 (m, |
| | 8H), 2.32-2.35 (m, 1H), 2.17-2.27 (m, 9H), 1.85-1.88 (m, 1H), 1.70-1.77 (m, |
| | 1H). |
| 420 | Cis-rac-N-(1-(2-(1,1-difluoroethyl)-6-methylpyrimidin-4-y1)-3-(3- |
| | (dimethylamino)-4-methylpyrrolidin-1-yl)-1H-pyrazolo[4,3-c]pyridin-6- |
| | yl)acetamide |
| | 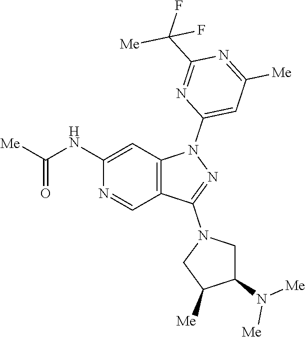 [SMILES: CC(=O)Nc1cc2c(cn1)c(N1C[C@@H](N(C)C)[C@@H](C)C1)nn2-c1cc(C)nc(C(C)(F)F)n1] |
| | SM: cis-rac-1-(6-chloro-1-(2-(1,1-difluoroethyl)-6-methylpyrimidin-4-y1)- |
| | 1H-pyrazolo[4,3-c]pyridin-3-yl)-N,N,4-trimethylpyrrolidin-3-amine |
| | (Preparation 667) |
| | prep-HPLC-F (30% to 60% MeCN) 5.0 mg, 23.8% yield as a white solid. |
| | LCMS m/z = 454.2 [M + H]⁺ ¹H NMR (400 MHz, CDCl₃) δ ppm: 9.47 (s, 1H), |
| | 8.72 (s, 1H), 8.07 (s, 1H), 7.64 (s, 1H), 3.86-3.90 (m, 2H), 3.58-3.65 (m, 2H), |
| | 2.53-2.73 (m, 5H), 2.24-2.34 (m, 12H), 1.11-1.13 (m, 3H). |
| 421 | N-(1-(2-(1,1-difluoroethyl)-6-methylpyrimidin-4-yl)-3-(3- |
| | (dimethylamino)azetidin-1-yl)-1H-pyrazolo[4,3-c]pyridin-6-yl)acetamide |
| | 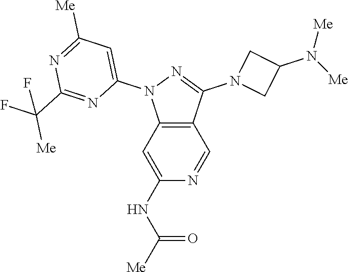 [SMILES: CC(=O)Nc1cc2c(cn1)c(N1CC(N(C)C)C1)nn2-c1cc(C)nc(C(C)(F)F)n1] |
| | SM: 1-(6-chloro-1-(2-(1,1-difluoroethyl)-6-methylpyrimidin-4-yl)-1H- |
| | pyrazolo[4,3-c]pyridin-3-yl)-N,N-dimethylazetidin-3-amine (Preparation 665) |
| | Prep-HPLC-P (25% to 55% MeCN) 30 mg, 35.5% yield as a yellow solid. |
| | LCMS m/z = 431.1 [M + H]⁺ ¹H NMR (400 MHz, CDCl₃) δ: ppm 9.44 (s, 1H), |
| | 8.53 (s, 1H), 8.02 (s, 1H), 7.62 (s, 1H), 4.37 (br s, 2H), 4.19 (br s, 2H), 3.45 |
| | (br s, 1H), 2.64 (s, 3H), 2.24-2.33 (m, 12H). |
| 422 | N-(1-(2-(1,1-difluoroethyl)-6-methoxypyrimidin-4-yl)-3-(3- |
| | (dimethylamino)azetidin-1-yl)-1H-pyrazolo[4,3-c]pyridin-6-yl)acetamide |
| | 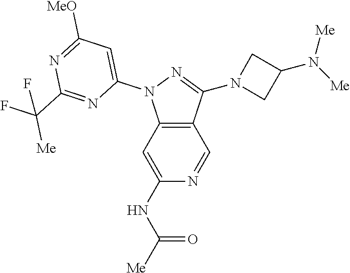 [SMILES: COc1cc(-n2nc(N3CC(N(C)C)C3)c3cnc(NC(C)=O)cc32)nc(C(C)(F)F)n1] |
| | SM: 1-(6-chloro-1-(2-(1,1-difluoroethyl)-6-methoxypyrimidin-4-y1)-1H- |
| | pyrazolo[4,3-c]pyridin-3-yl)-N,N-dimethylazetidin-3-amine (Preparation 691) |
| | Prep-HPLC-P (32% to 62% MeCN) 25 mg, 33.9% yield as a yellow solid. |
| | LCMS m/z = 477.2 [M + H]⁺ ¹H NMR (400 MHz, CDCl₃) δ: ppm 9.41 (s, 1H), |
| | 8.51 (s, 1H), 8.07 (s, 1H), 7.10 (s, 1H), 4.34 (t, J =7.2 Hz, 2H), 4.14-4.17 (m, |
| | 2H), 4.17 (s, 3H), 3.39-3.45 (m, 1H), 2.20-2.29 (m, 12H). |
| 423 | N-(1-(2-(1,1-difluoroethyl)-6-methylpyrimidin-4-yl)-3-((2-methoxy-2- |
| | methylpropyl)(methyl)amino)-1H-pyrazolo[4,3-c]pyridin-6-yl)acetamide |
| | 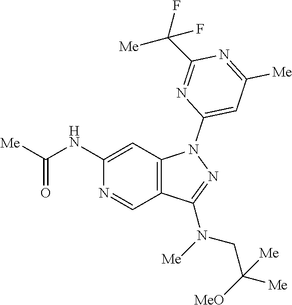 [SMILES: COC(C)(C)CN(C)c1nn(-c2cc(C)nc(C(C)(F)F)n2)c2cc(NC(C)=O)ncc12] |
| | SM: 6-chloro-1-(2-(1,1-difluoroethyl)-6-methylpyrimidin-4-yl)-N-(2- |
| | methoxy-2-methylpropyl)-N-methyl-1H-pyrazolo[4,3-c]pyridin-3-amine |
| | (Preparation 669) |
| | Prep-HPLC-C (42% to 68% MeCN) 22 mg, 34.8% yield as a white solid. |
| | LCMS m/z = 448.4 [M + H]⁺ ¹H NMR (400 MHz, CDCl₃) δ ppm 9.49 (s, 1H), |
| | 8.86 (s, 1H), 8.16 (s, 1H), 7.58 (s, 1H), 3.69 (s, 2H), 3.41 (s, 3H), 3.26 (s, |
| | 3H), 2.64 (s, 3H), 2.22-2.35 (m, 6H), 1.26 (s, 6H). |
| 424 | N-(1-(2-(1,1-difluoroethyl)-6-methylpyrimidin-4-yl)-3-((2- |
| | methoxyethyl)(methyl)amino)-1H-pyrazolo[4,3-c]pyridin-6-yl)acetamide |
| | 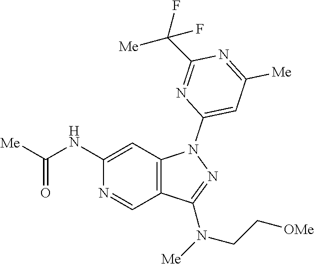 [SMILES: COCCN(C)c1nn(-c2cc(C)nc(C(C)(F)F)n2)c2cc(NC(C)=O)ncc12] |
| | 6-chloro-1-(2-(1, 1-difluoroethyl)-6-methylpyrimidin-4-y1)-N-(2- |
| | methoxyethyl)-N-methyl-1H-pyrazolo[4,3-c]pyridin-3-amine (Preparation |
| | 670) |
| | Prep-HPLC-C (35% to 65% MeCN) 45 mg, 50.1% yield as a white solid. |
| | LCMS m/z = 420.2 [M + H]⁺ ¹H NMR: (400 MHz, CDCl₃) δ ppm: 9.50 (s, |
| | 1H), 8.81 (s, 1H), 8.19 (s, 1H), 7.62 (s, 1H), 3.81 (t, J = 5.2 Hz, 2H), 3.72 (t, |
| | J = 5.2 Hz, 2H), 3.40 (s, 3H), 3.34 (s, 3H), 2.64 (s, 3H), 2.23-2.34 (m, 6H). |
| 425 | N-(3-(benzyl(methyl)amino)-1-(2-(1,1-difluoroethyl)-6-methylpyrimidin-4- |
| | y1)-1H-pyrazolo[4,3-c]pyridin-6-yl)acetamide |
| | 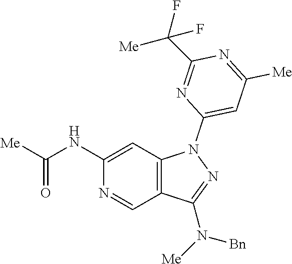 [SMILES: CC(=O)Nc1cc2c(cn1)c(N(C)Cc1ccccc1)nn2-c1cc(C)nc(C(C)(F)F)n1] |
| | SM: N-benzyl-6-chloro-1-(2-(1,1-difluoroethyl)-6-methylpyrimidin-4-yl)-N- |
| | methyl-1H-pyrazolo[4,3-c]pyridin-3-amine (Preparation 671) |
| | prep-HPLC-F (45% to 75% MeCN) |
| | 23.5 mg, 78.3% yield as a white solid. LCMS m/z = 452.1 [M + H]⁺ |
| 426 | N-(1-(2-(1,1-difluoroethyl)-6-methylpyrimidin-4-yl)-3-(3- |
| | (dimethylamino)piperidin-1-yl)-1H-pyrazolo[4,3-c]pyridin-6-yl)acetamide |
| | 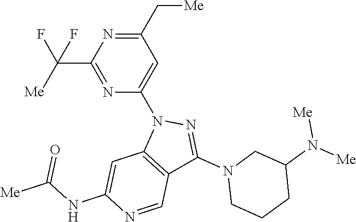 [SMILES: CCc1cc(-n2nc(N3CCCC(N(C)C)C3)c3cnc(NC(C)=O)cc32)nc(C(C)(F)F)n1] |
| | SM: 1-(6-chloro-1-(2-(1,1-difluoroethyl)-6-methylpyrimidin-4-yl)-1H- |
| | pyrazolo[4,3-c]pyridin-3-y1)-N,N-dimethylpiperidin-3-amine (Preparation |
| | 675) |
| | Prep-HPLC-C (31% to 61% MeCN). 17.0 mg, 80.8% yield as a white solid. |
| | LCMS m/z = 459.2 [M + H]⁺ ¹H NMR: (500 MHz, CDCl₃) δ ppm: 9.49 (s, |
| | 1H), 8.77 (s, 1H), 8.14 (s, 1H), 7.63 (s, 1H), 4.24-4.27 (m, 1H), 4.07 (d, |
| | J = 12.5 Hz, 1H), 3.00-3.07 (m, 2H), 2.64 (s, 3H), 2.39-2.41 (m, 1H), 2.32 (s, |
| | 6H), 2.26-2.28 (m, 6H), 2.23-2.25 (m, 1H), 1.75-1.78 (m, 1H), 1.74-1.76 (m, |
| | 2H). |
| 427 | N-(1-(2-(1,1-difluoroethyl)-6-methylpyrimidin-4-yl)-3-(4- |
| | (dimethylamino)piperidin-1-yl)-1H-pyrazolo[4,3-c]pyridin-6-yl)acetamide |
| | 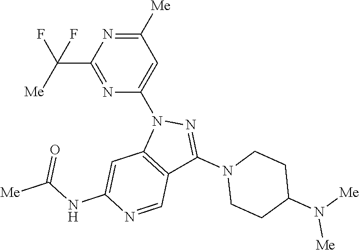 [SMILES: CC(=O)Nc1cc2c(cn1)c(N1CCC(N(C)C)CC1)nn2-c1cc(C)nc(C(C)(F)F)n1] |
| | SM: 1-(6-chloro-1-(2-(1, 1-difluoroethyl)-6-methylpyrimidin-4-yl)-1H- |
| | pyrazolo[4,3-c]pyridin-3-y1)-N,N-dimethylpiperidin-4-amine (Preparation |
| | 676) |
| | Prep-HPLC-P (35% to 65% MeCN) 35 mg, 37.0% yield as a yellow solid. |
| | LCMS m/z = 459.1 [M + H]⁺. ¹H NMR (500 MHz, CDCl₃) δ: ppm 9.50 (s, |
| | 1H), 8.77 (s, 1H), 8.04 (s, 1H), 7.65 (s, 1H), 4.23 (d, J = 12.5 Hz, 2H), 3.09- |
| | 3.13 (m, 2H), 2.62 (s, 3H), 2.33-2.35 (m, 1H), 2.25-2.29 (m, 12H), 2.02 (d, |
| | J = 13.0 Hz, 2H), 1.73-1.77 (m, 2H). |
| 428 | N-(1-(2-(1,1-difluoroethyl)-6-methylpyrimidin-4-y1)-3-(7-methyl-2,7- |
| | diazaspiro[4.4]nonan-2-y1)-1H-pyrazolo[4,3-c]pyridin-6-yl)acetamide |
| | 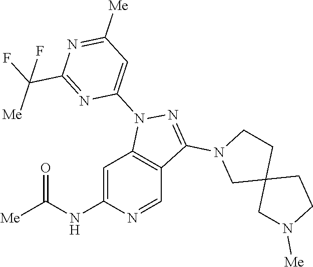 [SMILES: CC(=O)Nc1cc2c(cn1)c(N1CCC3(CCN(C)C3)C1)nn2-c1cc(C)nc(C(C)(F)F)n1] |
| | SM: 6-chloro-1-(2-(1,1-difluoroethyl)-6-methylpyrimidin-4-yl)-3-(7-methyl- |
| | 2,7-diazaspiro[4.4]nonan-2-yl)-1H-pyrazolo[4,3-c]pyridine (Preparation 677) |
| | Prep-HPLC-C (37% to 70% MeCN) 23 mg, 36.5% yield as white solid. |
| | LCMS m/z = 471.2 [M + H]⁺ ¹H NMR: (500 MHz, CDCl₃) δ ppm: 9.46 (s, 1H), |
| | 8.72 (s, 1H), 8.07 (s, 1H), 7.63 (s, 1H), 3.79 (m, 3H), 3.64-3.67 (m, 1H), 2.76 |
| | (m, 1H), 2.68-2.71 (m, 1H), 2.63-2.64 (m, 4H), 2.52-2.54 (m, 1H), 2.40 (s, 3H), |
| | 2.29 (m, 6H), 2.11 (m, 2H), 1.96 (m, 2H). |
| 429 | N-(1-(2-(1,1-difluoroethyl)-6-methylpyrimidin-4-yl)-3-(7-methyl-2,7- |
| | diazaspiro[3.5]nonan-2-yl)-1H-pyrazolo[4,3-c]pyridin-6-yl)acetamide |
| | 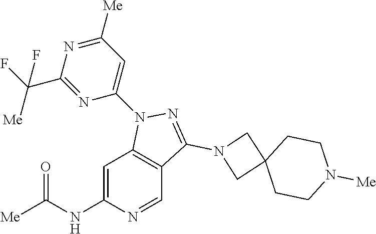 [SMILES: CC(=O)Nc1cc2c(cn1)c(N1CC3(CCN(C)CC3)C1)nn2-c1cc(C)nc(C(C)(F)F)n1] |
| | SM: 6-chloro-1-(2-(1,1-difluoroethyl)-6-methylpyrimidin-4-yl)-3-(7-methyl- |
| | 2,7-diazaspiro[3.5]nonan-2-yl)-1H-pyrazolo[4,3-c]pyridine (Preparation 678) |
| | prep-HPLC-C (35% to 63% MeCN) 25.0 mg, 85.0% yield as a white solid. |
| | LCMS m/z = 471.2 [M + H]⁺. ¹H NMR (400 MHz, MeOD-d₄) δ ppm: 9.44 (s, |
| | 1H), 8.53 (s, 1H), 8.04 (s, 1H), 7.61 (s, 1H), 4.04 (s, 4H), 2.64 (s, 3H), 2.40- |
| | 2.42 (m, 3H), 2.32-2.33 (m, 4H) 2.23-2.28 (m, 6H), 1.97 (br s, 4H). |
| 430 | N-(1-(2-(1,1-difluoroethyl)-6-methylpyrimidin-4-yl)-3-(6-methyl-2,6- |
| | diazaspiro[3.4]octan-2-yl)-1H-pyrazolo[4,3-c]pyridin-6-yl)acetamide |
| | 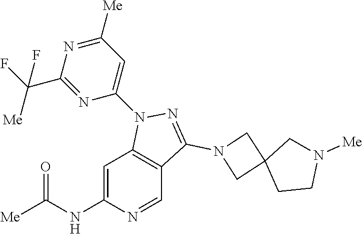 [SMILES: CC(=O)Nc1cc2c(cn1)c(N1CC3(CCN(C)C3)C1)nn2-c1cc(C)nc(C(C)(F)F)n1] |
| | SM: 6-chloro-1-(2-(1,1-difluoroethyl)-6-methylpyrimidin-4-yl)-3-(6-methyl- |
| | 2,6-diazaspiro[3.4]octan-2-y1)-1H-pyrazolo[4,3-c]pyridine (Preparation 679) |
| | Prep-HPLC-C (35% to 63% MeCN) 6.0 mg, 38.0% yield as a white solid. |
| | LCMS m/z = 457.3 [M + H]⁺ ¹H NMR (400 MHz, CDCl₃) δ ppm: 9.44 (s, 1H), |
| | 8.50 (s, 1H), 8.06 (s, 1H), 7.62 (s, 1H), 4.21-4.29 (m, 4H), 2.87(s, 2H), 2.59- |
| | 2.64 (m, 5H), 2.41 (s, 3H), 2.22-2.33 (m, 8H). |
| 431 | N-(1-(2-(1,1-difluoroethyl)-6-methylpyrimidin-4-yl)-3-(2-methyl-6-oxa-2,9- |
| | diazaspiro[4.5]decan-9-y1)-1H-pyrazolo[4,3-c]pyridin-6-yl)acetamide |
| | 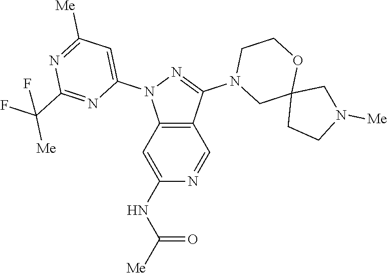 [SMILES: CC(=O)Nc1cc2c(cn1)c(N1CCOC3(CCN(C)C3)C1)nn2-c1cc(C)nc(C(C)(F)F)n1] |
| | SM: 9-(6-chloro-1-(2-(1, 1-difluoroethyl)-6-methylpyrimidin-4-yl)-1H- |
| | pyrazolo[4,3-c]pyridin-3-yl)-2-methyl-6-oxa-2,9-diazaspiro[4.5]decane |
| | (Preparation 680) |
| | Prep-HPLC-P (30% to 50% MeCN) 17 mg, 32.4% yield as a yellow solid. |
| | LCMS m/z = 487.3 [M + H]⁺ ¹H NMR: (400 MHz, CDCl₃) δ: ppm 9.51 (s, |
| | 1H), 8.76 (d, J = 0.8 Hz, 1H), 8.05 (s, 1H), 7.65 (s, 1H), 3.93-3.96 (m, 2H), |
| | 3.66-3.69 (m, 2H), 3.47-3.55 (m, 1H), 3.40-3.44 (m, 1H), 2.94-2.97 (m, 1H), |
| | 2.70-2.81 (m, 1H), 2.66 (s, 3H), 2.58-2.64 (m, 2H), 2.39 (s, 3H), 2.24-2.26 |
| | (m, 6H), 2.02-2.03 (m, 2H). |
| 432 | N-(1-(2-(1,1-difluoroethyl)-6-methylpyrimidin-4-yl)-3-(6-ethyl-3,6- |
| | diazabicyclo[3.1.1]heptan-3-yl)-1H-pyrazolo[4,3-c]pyridin-6-yl)acetamide |
| | 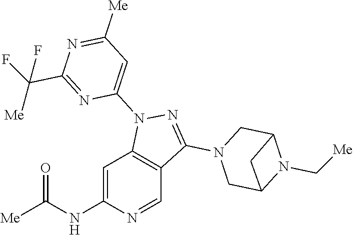 [SMILES: CCN1C2CC1CN(c1nn(-c3cc(C)nc(C(C)(F)F)n3)c3cc(NC(C)=O)ncc13)C2] |
| | SM: 3-(6-chloro-1-(2-(1,1-difluoroethyl)-6-methylpyrimidin-4-yl)-1H- |
| | pyrazolo[4,3-c]pyridin-3-y1)-6-ethyl-3,6-diazabicyclo[3.1.1]heptane |
| | (Preparation 693) |
| | Prep-HPLC-C (30% to 60% MeCN) 15 mg, 23.8% as a white solid. LCMS |
| | m/z = 457.1 [M + H]⁺ ¹H NMR (500 MHz, CDCl₃) δ ppm: 9.53 (s, 1H), 8.85 |
| | (s, 1H), 8.06 (s, 1H), 7.66 (s, 1H), 4.02-4.04 (m, 2H), 3.88-3.89 (m, 2H), |
| | 3.78-3.80 (m, 2H), 2.78 (br s, 1H), 2.65 (s, 3H), 2.58-2.60 (m, 2H), 2.26-2.33 |
| | (m, 6H), 1.77-1.79 (d, J = 4.5 Hz, 1H), 1.07-1.11 (m, 3H). |
| 433 | N-(1-(2-(1,1-difluoroethyl)-6-ethylpyrimidin-4-yl)-3-(6-ethyl-3,6- |
| | diazabicyclo[3.1.1]heptan-3-yl)-1H-pyrazolo[4,3-c]pyridin-6-yl)acetamide |
| | 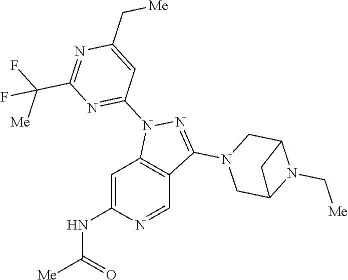 |
| | SM: 3-(6-chloro-1-(2-(1, 1-difluoroethyl)-6-ethylpyrimidin-4-yl)-1H- |
| | pyrazolo[4,3-c]pyridin-3-yl)-6-ethyl-3,6-diazabicyclo[3.1.1]heptane |
| | (Preparation 697) |
| | Prep-HPLC-C (48% to 78% MeCN) 130 mg, 61.9% yield as a white solid. |
| | LCMS m/z = 471.2 [M + H]⁺ ¹H NMR (400 MHz, CDCl₃) δ ppm: 9.53 (s, 1H), |
| | 8.85 (s, 1H), 8.07 (s, 1H), 7.66 (s, 1H), 4.03 (d, J = 11.2 Hz, 2H), 3.87 (d, |
| | J = 5.6 Hz, 2H), 3.78 (d, J = 11.2 Hz, 2H), 2.90 (q, J = 7.6 Hz, 2H), 2.73-2.78 (m, |
| | 1H), 2.55-2.61 (m, 2H), 2.34-2.24 (m, 6H), 1.77 (d, J = 8.8 Hz, 1H), 1.38 (t, |
| | J = 7.6 Hz, 3H), 1.08 (t, J = 7.2 Hz, 3H). |
| 434 | N-(1-(6-(1,1-difluoroethyl)-4-((1s,3R)-3-methoxycyclobutoxy)pyridin-2-yl)- |
| | 3-((S)-3-(dimethylamino)pyrrolidin-1-yl)-1H-pyrazolo[4,3-c]pyridin-6- |
| | yl)acetamide |
| | 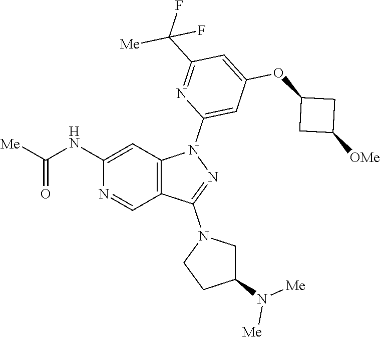 [SMILES: CO[C@H]1C[C@@H](Oc2cc(-n3nc(N4CC[C@H](N(C)C)C4)c4cnc(NC(C)=O)cc43)nc(C(C)(F)F)c2)C1] |
| | SM: (S)-1-(6-chloro-1-(6-(1,1-difluoroethyl)-4-((1s,3R)-3- |
| | methoxycyclobutoxy)pyridin-2-y1)-1H-pyrazolo[4,3-c]pyridin-3-y1)-N,N- |
| | dimethylpyrrolidin-3-amine (Preparation 688) |
| | prep-HPLC-C (45% to 75% MeCN) 28.0 mg, 33.5% yield as a white solid. |
| | LCMS m/z = 530.2 [M + H]⁺ ¹H NMR (500 MHz, CDCl₃) δ ppm: 9.35 (s, 1H), |
| | 8.70 (s, 1H), 8.06 (s, 1H), 7.34 (s, 1H), 6.90 (s, 1H), 4.50-4.53 (m, 1H), 3.92- |
| | 3.94 (m, 2H), 3.72-3.76 (m, 2H), 3.55-3.57 (m, 1H), 3.29 (s, 3H), 2.94-3.00 |
| | (m, 3H), 2.39 (s, 6H), 2.24-2.28 (m, 7H), 2.17-2.21 (m, 2H), 1.90-2.10 (m, |
| | 1H). |

### Example 435: N-(1-(2-(1,1-difluoroethyl)-6-ethylpyrimidin-4-yl)-3-(3-(dimethylamino)pyrrolidin-1-yl)-1H-pyrazolo[4,3-c]pyridin-6-yl)propionamide

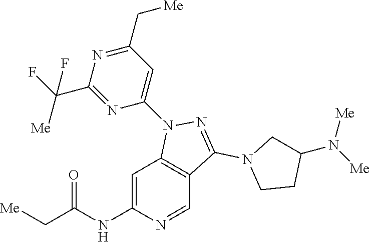 [SMILES: CCC(=O)Nc1cc2c(cn1)c(N1CCC(N(C)C)C1)nn2-c1cc(CC)nc(C(C)(F)F)n1]

N-(1-(2-(1,1-Difluoroethyl)-6-ethylpyrimidin-4-yl)-3-(3-(dimethylamino)pyrrolidin-1-yl)-1H-pyrazolo[4,3-c]pyridin-6-yl)propionamide was obtained as a white solid, 24 mg, 44.3%, from 1-(6-chloro-1-(2-(1,1-difluoroethyl)-6-ethylpyrimidin-4-yl)-1H-pyrazolo[4,3-c]pyridin-3-yl)-N,N-dimethylpyrrolidin-3-amine (Preparation 700) and propionamide, following a similar procedure to that described in Example 405. The crude was purified by prep-HPLC-F (37% to 65% MeCN). LCMS m/z=473.2 [M+H]^{+ 1}H NMR (500 MHz, CDCl₃) δ ppm: 9.47 (s, 1H), 8.70 (s, 1H), 8.14-8.18 (m, 1H), 7.62 (s, 1H), 3.93-3.96 (m, 2H), 3.73-3.75 (m, 1H), 3.51-3.54 (m, 1H), 2.88-2.92 (m, 3H), 2.47-2.50 (m, 2H), 2.44-2.64 (m, 6H), 2.25-2.37 (m, 4H), 2.04-2.08 (m, 1H), 1.37 (t, J=7.5 Hz, 3H), 1.29 (t, J=7.5 Hz, 3H).

### Example 436: methyl (1-(2-(1,1-difluoroethyl)-6-ethylpyrimidin-4-yl)-3-(3-(dimethylamino)pyrrolidin-1-yl)-1H-pyrazolo[4,3-c]pyridin-6-yl)carbamate

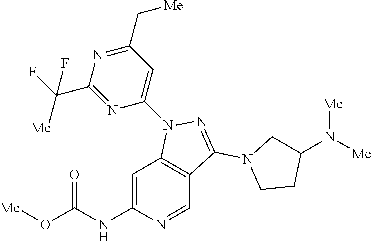 [SMILES: CCc1cc(-n2nc(N3CCC(N(C)C)C3)c3cnc(NC(=O)OC)cc32)nc(C(C)(F)F)n1]

To a solution of 1-(6-chloro-1-(2-(1,1-difluoroethyl)-6-ethylpyrimidin-4-yl)-1H-pyrazolo[4,3-c]pyridin-3-yl)-N,N-dimethylpyrrolidin-3-amine (Preparation 700, 150 mg, 0.344 mmol) in dioxane (5.0 mL) was added methyl carbamate (129.2 mg, 1.72 mmol), BrettPhos Pd G₃ (31.2 mg, 34.41 μmol) and Cs₂CO₃ (336.4 mg, 1.03 mmol) and the reaction was stirred at 100° C. for 2 h under N₂. The reaction was concentrated and diluted with H₂O (20 mL). The mixture was extracted with DCM/MeOH=10/1 (2×20 mL). The combined organic phase was concentrated under reduced pressure to give the crude product (50 mg, crude) as a yellow solid. The crude was concentrated and purified by prep-HPLC-C (43% to 67% MeCN) to give methyl (1-(2-(1,1-difluoroethyl)-6-ethylpyrimidin-4-yl)-3-(3-(dimethylamino)pyrrolidin-1-yl)-1H-pyrazolo[4,3-c]pyridin-6-yl)carbamate (11.0 mg, 6.70 yield) as a white solid. LCMS m/z=475.2 [M+H]⁺. ¹H NMR (500 MHz, CDCl₃) δ ppm: 9.23 (s, 1H), 8.74 (s, 1H), 8.12 (s, 1H), 7.63 (s, 1H), 3.93-3.98 (m, 2H), 3.87 (s, 3H), 3.75-3.76 (m, 1H), 3.57-3.59 (m, 1H), 2.88-2.93 (m, 3H), 2.40 (s, 6H), 2.21-2.33 (m, 4H), 2.05-2.08 (m, 1H), 1.38 (t, J=7.5 Hz, 3H).

Examples 437 to 439: The compounds in the following table were prepared from the appropriate 6-chloro-1H-pyrazolo[4,3-c]pyridine and acetamide, following a similar procedure to that described in Example 436.

| Example No | Name, Structure, Starting Materials (SM) and Data |
|---|---|
| 437 | methyl (1-(2-(1,1-difluoroethyl)-6-ethylpyrimidin-4-yl)-3-(6- |
| | methyl-3,6-diazabicyclo[3.1.1]heptan-3-yl)-1H-pyrazolo[4,3-c]pyridin-6- |
| | yl)carbamate |
| | 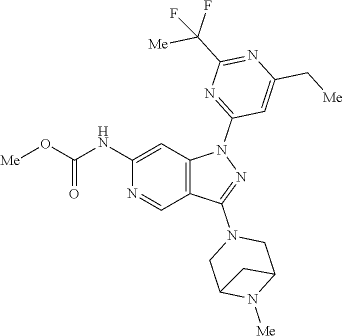 [SMILES: CCc1cc(-n2nc(N3CC4CC(C3)N4C)c3cnc(NC(=O)OC)cc32)nc(C(C)(F)F)n1] |
| | SM: 3-(6-chloro-1-(2-(1, 1-difluoroethyl)-6-ethylpyrimidin-4-yl)- |
| | 1H-pyrazolo[4,3-c]pyridin-3-yl)-6-methyl-3,6-diazabicyclo[3.1.1]heptane |
| | (Preparation 696) and methyl carbamate |
| | Prep-HPLC-F (43% to 73% MeCN). 15 mg, 13.8% yield as a |
| | white solid. LCMS m/z = 473.2 [M + H]+ 1H NMR (400 MHz, CDCl3) 8 |
| | ppm: 9.28 (s, 1H), 8.85 (s, 1H), 7.66 (s, 1H), 7.64 (s, 1H), 4.08 (d, J=10.8 |
| | Hz, 2H), 3.82-3.88 (m, 7H), 2.91 (d, J=7.5 Hz, 2H), 2.81 (br s, 1H), 2.21- |
| | 2.33 (m, 6H), 1.79 (d, J=9.6 Hz, 1H), 1.37-1.41 (m, 3H) |
| 438 | N-(1-(2-(1,1-difluoroethyl)-6-ethylpyrimidin-4-yl)-3-(6-methyl- |
| | 3,6-diazabicyclo[3.1.1]heptan-3-yl)-1H-pyrazolo[4,3-c]pyridin-6- |
| | yl)propionamide |
| | 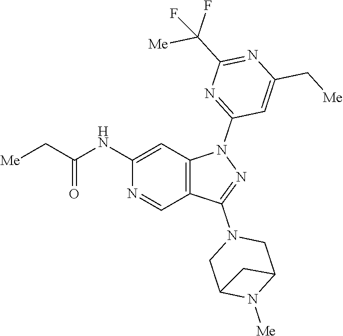 [SMILES: CCC(=O)Nc1cc2c(cn1)c(N1CC3CC(C1)N3C)nn2-c1cc(CC)nc(C(C)(F)F)n1] |
| | SM: 3-(6-chloro-1-(2-(1,1-difluoroethyl)-6-ethylpyrimidin-4-yl)- |
| | 1H-pyrazolo[4,3-c]pyridin-3-yl)-6-methyl-3,6-diazabicyclo[3.1.1]heptane |
| | (Preparation 696) and propionamide |
| | Prep-HPLC-C (33% to 63% MeCN). 25 mg, 38.4% as a white |
| | solid. LCMS m/z = 471.2 [M + H]⁺. ¹H NMR: (500 MHz, CDCl₃) δ ppm: |
| | 9.54 (s, 1H), 8.86 (s, 1H), 8.01 (s, 1H), 7.66 (s, 1H), 4.06 (d, J = 7.5 Hz, |
| | 2H), 3.78-3.81 (m, 4H), 2.91 (t, J = 7.5 Hz, 2H), 2.76 (br s, 1H), 2.45-2.54 |
| | (m, 2H), 2.26-2.36 (m, 6H), 1.76 (d, J = 8.5 Hz, 1H), 1.38 (t, J = 7.5 Hz, |
| | 3H), 1.31 (t, J = 7.5 Hz, 3H). |
| 439 | N-(1-(2-(1,1-difluoroethyl)-6-ethylpyrimidin-4-yl)-3-(3- |
| | (dimethylamino)-3-methylpyrrolidin-1-yl)-1H-pyrazolo[4,3-c]pyridin-6- |
| | y1)propionamide |
| | 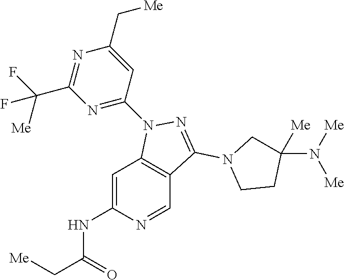 [SMILES: CCC(=O)Nc1cc2c(cn1)c(N1CCC(C)(N(C)C)C1)nn2-c1cc(CC)nc(C(C)(F)F)n1] |
| | SM: 1-(6-chloro-1-(2-(1,1-difluoroethyl)-6-ethylpyrimidin-4-yl)- |
| | 1H-pyrazolo[4,3-c]pyridin-3-y1)-N,N,3-trimethylpyrrolidin-3-amine |
| | (Preparation 703) and propionamide |
| | Prep-HPLC-C (49% to 69% MeCN). 36.1 mg, 33.4% yield as a |
| | white solid. LCMS m/z = 487.2 [M + H]⁺ ¹H NMR (500 MHz, CDCl₃) δ |
| | ppm: 9.48 (s, 1H), 8.71 (s, 1H), 8.04 (s, 1H), 7.63 (s, 1H), 3.94-3.95 (m, |
| | 1H), 3.84-3.86 (m, 1H), 3.67-3.68 (m, 2H), 2.90 (q, J = 8.0 Hz, 2H), 2.47 |
| | (q, J = 7.5 Hz, 3H), 2.37 (s, 6H), 2.30 (t, J = 19.0 Hz, 3H), 2.22-2.25 (m, |
| | 1H), 2.19-2.21 (m, 1H), 1.38 (t, J = 7.5 Hz, 3H), 1.30 (t, J = 7.5 Hz, 3H), |
| | 1.17 (s, 3H). |

### Example 440: N-(1-(2-(1,1-difluoroethyl)pyrimidin-4-yl)-3-(2-methyl-4-(methylamino)pyrrolidin-1-yl)-1H-pyrazolo[4,3-c]pyridin-6-yl)acetamide

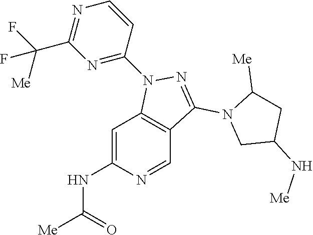 [SMILES: CNC1CC(C)N(c2nn(-c3ccnc(C(C)(F)F)n3)c3cc(NC(C)=O)ncc23)C1]

Step 1: To a solution of tert-butyl (1-(6-chloro-1-(2-(1,1-difluoroethyl)pyrimidin-4-yl)-1H-pyrazolo[4,3-c]pyridin-3-yl)-5-methylpyrrolidin-3-yl)(methyl)carbamate (Preparation 704, 30 mg, 59.1 μmol) in dioxane (2 mL) was added acetamide (7.0 mg, 0.118 mmol), Cs₂CO₃ (38.5 mg, 0.118 mmol) and BrettPhos Pd G3 (5.4 mg, 5.91 μmol) and the reaction was stirred at 100° C. for 16 h under N₂. The reaction mixture was filtered and the filtrate concentrated to give tert-butyl (1-(6-acetamido-1-(2-(1,1-difluoroethyl)pyrimidin-4-yl)-1H-pyrazolo[4,3-c]pyridin-3-yl)-5-methylpyrrolidin-3-yl)(methyl)carbamate (30 mg, crude) as a yellow solid.

Step 2: To a solution of tert-butyl (1-(6-acetamido-1-(2-(1,1-difluoroethyl)pyrimidin-4-yl)-1H-pyrazolo[4,3-c]pyridin-3-yl)-5-methylpyrrolidin-3-yl)(methyl)carbamate (30 mg, 0.057 mmol) in DCM (2 mL) was added HCl/Dioxane (4 M, 0.5 mL) and the reaction stirred at 25° C. for 16 h. The mixture was adjusted pH to 7 with NH₄OH then concentrated and was purified by Prep-HPLC-P (20% to 50% MeCN) to give N-(1-(2-(1,1-difluoroethyl)pyrimidin-4-yl)-3-(2-methyl-4-(methylamino)pyrrolidin-1-yl)-1H-pyrazolo[4,3-c]pyridin-6-yl)acetamide (3 mg, 12.3% yield) as a yellow solid. LCMS m/z=431.1 [M+H]⁺. ¹H NMR (400 MHz, MeOD-d₄) δ: ppm 9.41-9.42 (m, 1H), 8.87-8.88 (m, 1H), 8.75-8.77 (m, 1H), 7.85-7.88 (m, 1H), 4.35-4.63 (m, 1H), 4.20-4.23 (m, 1H), 3.82-3.93 (m, 2H), 2.77-2.79 (m, 3H), 2.19-2.29 (m, 6H) 1.85-1.88 (m, 1H) 1.40-1.55 (m, 3H) 1.29-1.33 (m, 1H).

### Example 441: N-(1-(2-(1,1-difluoroethyl)-6-methylpyrimidin-4-yl)-3-(3-(methylamino)pyrrolidin-1-yl)-1H-pyrazolo[4,3-c]pyridin-6-yl)acetamide trifluoroacetate

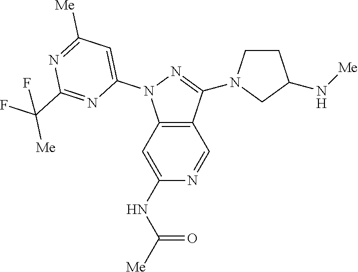 [SMILES: CNC1CCN(c2nn(-c3cc(C)nc(C(C)(F)F)n3)c3cc(NC(C)=O)ncc23)C1]

Step 1: To a solution of benzyl (1-(6-chloro-1-(2-(1,1-difluoroethyl)-6-methylpyrimidin-4-yl)-1H-pyrazolo[4,3-c]pyridin-3-yl)pyrrolidin-3-yl)(methyl)carbamate (Preparation 664, 400 mg, 0.738 mmol) and acetamide (218.0 mg, 3.69 mmol) in dioxane (11 mL) was added BrettPhos Pd G3 (66.9 mg, 0.074 mmol) and Cs₂CO₃ (480.9 mg, 1.48 mmol) and the reaction was stirred at 100° C. for 2 h under N₂. The mixture was concentrated, water (30 mL) was added and the aqueous mixture was extracted with EtOAc (30 mL×2). The combined organic layers were washed with brine (30 mLx 2), dried over Na₂SO₄, filtered and concentrated under vacuum. The crude was purified on silica gel column chromatography (PE/EtOAc=1/0 to 3/7) to yield benzyl (1-(6-acetamido-1-(2-(1,1-difluoroethyl)-6-methylpyrimidin-4-yl)-1H-pyrazolo[4,3-c]pyridin-3-yl)pyrrolidin-3-yl)(methyl)carbamate (247.0 mg, 59.3% yield) as a yellow solid.

Step 2: A solution of benzyl (1-(6-acetamido-1-(2-(1,1-difluoroethyl)-6-methylpyrimidin-4-yl)-1H-pyrazolo[4,3-c]pyridin-3-yl)pyrrolidin-3-yl)(methyl)carbamate (200 mg, 0.354 mmol) in TFA (5.0 mL) was stirred at 50° C. for 3 h. The mixture was concentrated under vacuum to get the crude, which was purified by prep-HPLC-Q (14% to 34% MeCN) to give N-(1-(2-(1,1-difluoroethyl)-6-methylpyrimidin-4-yl)-3-(3-(methylamino)pyrrolidin-1-yl)-1H-pyrazolo[4,3-c]pyridin-6-yl)acetamide trifluoroacetate (78.0 mg, 51.2% yield) as yellow solid. LCMS m/z=431.3 [M+H]¹H NMR: (400 MHz, CDCl₃) δ ppm: 9.13 (s, 1H), 8.51 (s, 1H), 7.43 (s, 1H), 3.80-4.04 (m, 4H), 3.55 (br s, 1H), 2.81 (s, 3H), 2.60 (s, 3H), 2.43 (br s, 2H), 2.28-2.29 (m, 1H), 2.27 (s, 3H), 2.13-2.22 (m, 3H).

### Example 442: N-(1-(2-(1,1-difluoroethyl)pyrimidin-4-yl)-3-(3-methyl-4-(methylamino)pyrrolidin-1-yl)-1H-pyrazolo[4,3-c]pyridin-6-yl)acetamide trifluoroacetate

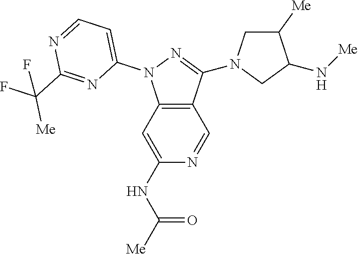 [SMILES: CNC1CN(c2nn(-c3ccnc(C(C)(F)F)n3)c3cc(NC(C)=O)ncc23)CC1C]

Step 1: N-(1-(2-(1,1-difluoroethyl)pyrimidin-4-yl)-3-(3-((4-methoxybenzyl)(methyl)amino)-4-methylpyrrolidin-1-yl)-1H-pyrazolo[4,3-c]pyridin-6-yl)acetamide was obtained as a white solid, 145 mg, 79%, from 1-(6-chloro-1-(2-(1,1-difluoroethyl)pyrimidin-4-yl)-1H-pyrazolo[4,3-c]pyridin-3-yl)-N-(4-methoxybenzyl)-N,4-dimethylpyrrolidin-3-amine (Preparation 657) and acetamide, following a similar procedure to that described in Example 441, step 1.

Step 2: A solution of N-(1-(2-(1,1-difluoroethyl)pyrimidin-4-yl)-3-(3-((4-methoxybenzyl)(methyl)amino)-4-methylpyrrolidin-1-yl)-1H-pyrazolo[4,3-c]pyridin-6-yl)acetamide (100 mg, 0.182 mmol) in TFA (3 mL, 39.18 mmol) was stirred at 130° C. for 4 h under microwave. The mixture was quenched with H₂O (10 mL) and extracted with EtOAc (10 mL×3). The combined organic layers were washed with brine (20 mL), dried over Na₂SO₄, filtered and concentrated under vacuum to give the crude, which was purified by prep-HPLC-C (30% to 55% MeCN) (50.0 mg, crude) and further purified by prep-HPLC-Q (14% to 44% MeCN) to give N-(1-(2-(1,1-difluoroethyl)pyrimidin-4-yl)-3-(3-methyl-4-(methylamino)pyrrolidin-1-yl)-1H-pyrazolo[4,3-c]pyridin-6-yl)acetamide trifluoroacetate (23.0 mg, 46.0% yield) as a white solid. LCMS m/z=431.1 [M+H]⁺. HNMR (500 MHz, MeOD-d₄) δ ppm: 9.25 (s, 1H), 8.80 (s, 1H), 8.70 (d, J=6.0 Hz, 1H), 8.46 (br s, 1H), 7.74 (d, J=5.5 Hz, 1H), 3.86-3.99 (m, 4H), 3.58-3.59 (m, 1H), 2.85-2.89 (m, 1H), 2.81 (s, 3H), 2.17-2.25 (m, 6H), 1.28 (d, J=7.0 Hz, 3H).

### Example 443 and 444: (R)—N-(1-(2-(1,1-difluoroethyl)-6-methylpyrimidin-4-yl)-3-(3-(dimethylamino)-3-methylpyrrolidin-1-yl)-1H-pyrazolo[4,3-c]pyridin-6-yl)propionamide and (S)—N-(1-(2-(1,1-difluoroethyl)-6-methylpyrimidin-4-yl)-3-(3-(dimethylamino)-3-methylpyrrolidin-1-yl)-1H-pyrazolo[4,3-c]pyridin-6-yl)propionamide

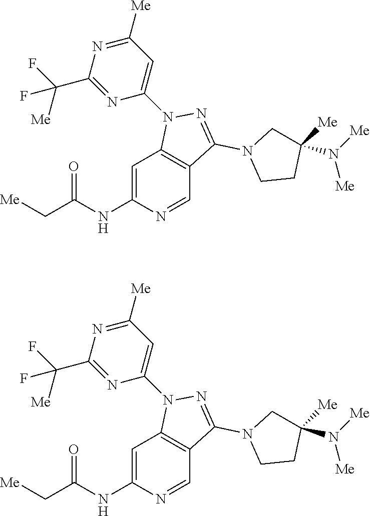 [SMILES: CCC(=O)Nc1cc2c(cn1)c(N1CC[C@@](C)(N(C)C)C1)nn2-c1cc(C)nc(C(C)(F)F)n1 ; CCC(=O)Nc1cc2c(cn1)c(N1CC[C@](C)(N(C)C)C1)nn2-c1cc(C)nc(C(C)(F)F)n1]

To a solution of 1-(6-chloro-1-(2-(1,1-difluoroethyl)-6-methylpyrimidin-4-yl)-1H-pyrazolo[4,3-c]pyridin-3-yl)-N,N,3-trimethylpyrrolidin-3-amine (Preparation 701, 150 mg, 0.344 mmol) in dioxane (4 mL) was added propionamide (75.5 mg, 1.03 mmol), BrettPhos Pd G3 (31.2 mg, 34.4 μmol) and Cs₂CO₃ (224.2 mg, 0.688 mmol) and the reaction was stirred at 100° C. for 16 h under N₂. The mixture was concentrated and was purified by Prep-HPLC-P (45% to 65% MeCN) to give N-(1-(2-(1,1-difluoroethyl)-6-methylpyrimidin-4-yl)-3-(3-(dimethylamino)-3-methylpyrrolidin-1-yl)-1H-pyrazolo[4,3-c]pyridin-6-yl)propionamide (70 mg, 43.1% yield) as a yellow solid.

This was separated by SFC (Column: DAICEL CHIRALPAK AD(250 mm*30 mm,10 um); Mobile Phase: 50% of 0.1% NH₃·H₂O MeOH; Flow Rate(mL/min): 80; Column temp: 35° C. to give Peak 1, enantiomer 1, (R)—N-(1-(2-(1,1-difluoroethyl)-6-methylpyrimidin-4-yl)-3-(3-(dimethylamino)-3-methylpyrrolidin-1-yl)-1H-pyrazolo[4,3-c]pyridin-6-yl)propionamide or (S)—N-(1-(2-(1,1-difluoroethyl)-6-methylpyrimidin-4-yl)-3-(3-(dimethylamino)-3-methylpyrrolidin-1-yl)-1H-pyrazolo[4,3-c]pyridin-6-yl)propionamide (30 mg, 42.9% yield) as a white solid. LCMS m/z=473.2 [M+H]^{+ 1}H NMR: (500 MHz, MeOD-d₄) δ: ppm 9.36 (s, 1H), 8.85 (s, 1H), 7.71 (s, 1H), 3.96-3.98 (m, 1H), 3.82-3.85 (m, 1H), 3.76 (d, J=9.5 Hz, 1H), 3.63 (d, J=9.5 Hz, 1H), 2.60 (s, 3H), 2.47-2.52 (m, 2H), 2.38 (s, 6H), 2.21-2.28 (m, 3H), 2.11-2.15 (m, 2H), 1.22-1.27 (m, 6H).

and Peak 2, enantiomer 2, (S)—N-(1-(2-(1,1-difluoroethyl)-6-methylpyrimidin-4-yl)-3-(3-(dimethylamino)-3-methylpyrrolidin-1-yl)-1H-pyrazolo[4,3-c]pyridin-6-yl)propionamide or (R)—N-(1-(2-(1,1-difluoroethyl)-6-methylpyrimidin-4-yl)-3-(3-(dimethylamino)-3-methylpyrrolidin-1-yl)-1H-pyrazolo[4,3-c]pyridin-6-yl)propionamide (30 mg, 42.9% yield) both as a white solid. LCMS m/z=473.2 [M+H]⁺. ¹H NMR: (500 MHz, MeOD-d₄) δ: ppm 9.34 (s, 1H), 8.84 (s, 1H), 7.69 (s, 1H), 3.95-3.96 (m, 1H), 3.81-3.84 (m, 1H), 3.75 (d, J=9.5 Hz, 1H), 3.62 (d, J=9.5 Hz, 1H), 2.60 (s, 3H), 2.47-2.52 (m, 2H), 2.38 (s, 6H), 2.20-2.28 (m, 3H), 2.11-2.15 (m, 2H), 1.22-1.27 (m, 6H).

### Example 445: N-(1-(2-(1,1-difluoroethyl)-6-methylpyrimidin-4-yl)-3-(ethylamino)-1H-pyrazolo[4,3-c]pyridin-6-yl)acetamide

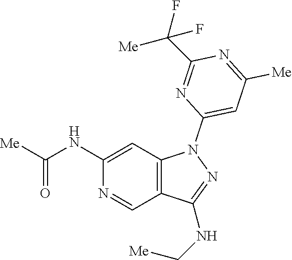 [SMILES: CCNc1nn(-c2cc(C)nc(C(C)(F)F)n2)c2cc(NC(C)=O)ncc12]

Step 1: N-(3-(benzyl(ethyl)amino)-1-(2-(1,1-difluoroethyl)-6-methylpyrimidin-4-yl)-1H-pyrazolo[4,3-c]pyridin-6-yl)acetamide was obtained as a yellow solid, 240 mg, 77.4%, from N-benzyl-6-chloro-1-(2-(1,1-difluoroethyl)-6-methylpyrimidin-4-yl)-N-ethyl-1H-pyrazolo[4,3-c]pyridin-3-amine (Preparation 673) and acetamide, following the procedure described in Example 441, step 1. LCMS m/z=466.2 [M+H]⁺ Step 2: To a solution of N-(3-(benzyl(ethyl)amino)-1-(2-(1,1-difluoroethyl)-6-methylpyrimidin-4-yl)-1H-pyrazolo[4,3-c]pyridin-6-yl)acetamide (100 mg, 0.21 mmol) in MeOH (5.0 mL) was added Pd/C (45.7 mg, 0.04 mmol, 10% purity). The mixture was stirred at 66° C. for 16 h under H₂ (15 psi). The mixture was concentrated and purified by prep-HPLC-C (35% to 70% MeCN) to give N-(1-(2-(1,1-difluoroethyl)-6-methylpyrimidin-4-yl)-3-(ethylamino)-1H-pyrazolo[4,3-c]pyridin-6-yl)acetamide

(25.2 mg, 31.3% yield) as a white solid. LCMS m/z=376.4 [M+H]⁺. HNMR: (400 MHz, CDCl₃) δ: ppm 9.43 (s, 1H), 8.54 (s, 1H), 8.12 (s, 1H), 7.62 (s, 1H), 4.39-4.42 (m, 1H), 3.54-3.61 (m, 2H), 2.64 (s, 3H), 2.23-2.33 (m, 6H), 1.39 (t, J=7.2 Hz, 3H).

### Example 446: N-(3-amino-1-(2-(1,1-difluoroethyl)-6-methylpyrimidin-4-yl)-1H-pyrazolo[4,3-c]pyridin-6-yl)acetamide

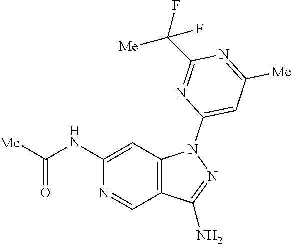 [SMILES: CC(=O)Nc1cc2c(cn1)c(N)nn2-c1cc(C)nc(C(C)(F)F)n1]

Step 1: N-(3-(Benzylamino)-1-(2-(1,1-difluoroethyl)-6-methylpyrimidin-4-yl)-1H-pyrazolo[4,3-c]pyridin-6-yl)acetamide was obtained as a yellow solid, from N-benzyl-6-chloro-1-(2-(1,1-difluoroethyl)-6-methylpyrimidin-4-yl)-1H-pyrazolo[4,3-c]pyridin-3-amine (Preparation 672) and acetamide, following the procedure described in Example 441, step 1.

Step 2: To a solution of N-(3-(benzylamino)-1-(2-(1,1-difluoroethyl)-6-methylpyrimidin-4-yl)-1H-pyrazolo[4,3-c]pyridin-6-yl)acetamide (20 mg, 0.041 mmol) in AcOH (2 mL) was added Pd(OH)₂ (10 mg, 0.071 mmol) under N₂ atmosphere. The mixture was stirred under H₂ (50 psi) at 50° C. for 16 h. The reaction mixture was filtered and concentrated under reduced pressure to give a residue, which was purified by Prep HPLC-S (12% to 42% MeCN) to give N-(3-amino-1-(2-(1,1-difluoroethyl)-6-methylpyrimidin-4-yl)-1H-pyrazolo[4,3-c]pyridin-6-yl)acetamide (11.7 mg, 52.2% yield) as a yellow oil. LCMS m/z=347.8 [M+H]⁺. 1H NMR (400 MHz, MeOD-d₄) δ ppm 9.30 (s, 1H), 8.76 (s, 1H), 7.66 (s, 1H), 2.59 (s, 3H), 2.18-2.25 (m, 6H)

### Example 447: N-(1-(2-(1,1-difluoroethyl)-6-methylpyrimidin-4-yl)-3-(methylamino)-1H-pyrazolo[4,3-c]pyridin-6-yl)acetamide

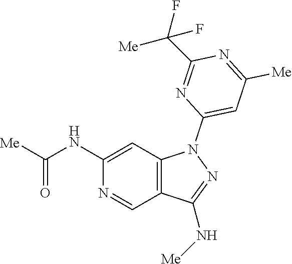 [SMILES: CNc1nn(-c2cc(C)nc(C(C)(F)F)n2)c2cc(NC(C)=O)ncc12]

To a solution of N-(3-(benzyl(methyl)amino)-1-(2-(1,1-difluoroethyl)-6-methylpyrimidin-4-yl)-1H-pyrazolo[4,3-c]pyridin-6-yl)acetamide (Example 425, 30.0 mg, 0.066 mmol) in MeOH (5 mL) was added Pd/C (14.14 mg, 10% purity) under H₂ (15 psi) and the reaction was stirred at 60° C. for 20 h. The mixture was filtered, concentrated and purified by prep-HPLC-C (20% to 60% MeCN) to give N-(1-(2-(1,1-difluoroethyl)-6-methylpyrimidin-4-yl)-3-(methylamino)-1H-pyrazolo[4,3-c]pyridin-6-yl)acetamide (8.5 mg, 35.4% yield) as a white solid. LCMS m/z=362.1 [M+H]⁺. ¹H NMR (500 MHz, CDCl₃) δ ppm: 9.44 (s, 1H), 8.52 (s, 1H), 8.07 (s, 1H), 7.67 (s, 1H), 4.47 (d, J=5.0 Hz, 1H), 3.17 (d, J=5.0 Hz, 3H), 2.65 (s, 3H), 2.24-2.32 (m, 6H).

### Example 448: methyl 6-acetamido-1-(6-(1,1-difluoroethyl)pyridin-2-yl)-1H-pyrazolo[4,3-c]pyridine-3-carboxylate

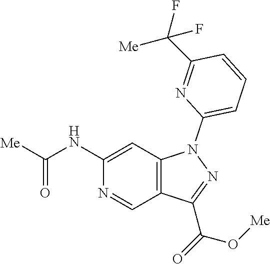 [SMILES: COC(=O)c1nn(-c2cccc(C(C)(F)F)n2)c2cc(NC(C)=O)ncc12]

Methyl 6-acetamido-1-(6-(1,1-difluoroethyl)pyridin-2-yl)-1H-pyrazolo[4,3-c]pyridine-3-carboxylate was obtained as a white powder from methyl 6-chloro-1-(6-(1,1-difluoroethyl)pyridin-2-yl)-1H-pyrazolo[4,3-c]pyridine-3-carboxylate (Preparation 706) and acetamide, following a similar procedure to that described in Example 445, step 1. LCMS m/z=376 [M+H]^{+ 1}H NMR (400 MHz, CDCl₃) δ 9.62 (s, 1H), 9.24 (d, J=1.00 Hz, 1H), 8.90 (br s, 1H), 8.26 (d, J=8.28 Hz, 1H), 8.05 (t, J=7.91 Hz, 1H), 7.70 (d, J=7.53 Hz, 1H), 4.13 (s, 3H), 2.26-2.41 (m, 6H).

### Example 449: N-(3-(azetidine-1-carbonyl)-1-(6-(1,1-difluoroethyl)pyridin-2-yl)-1H-pyrazolo[4,3-c]pyridin-6-yl)acetamide

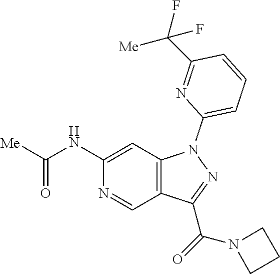 [SMILES: CC(=O)Nc1cc2c(cn1)c(C(=O)N1CCC1)nn2-c1cccc(C(C)(F)F)n1]

Step 1: To a solution of methyl 6-acetamido-1-(6-(1,1-difluoroethyl)pyridin-2-yl)-1H-pyrazolo[4,3-c]pyridine-3-carboxylate (Example 448, 100 mg, 0.266 mmol) in MeOH (1 mL) and THF (1 mL) was added aq. NaOH (3 M, 0.2 mL) and the reaction mixture stirred at 35° C. for 1 h. HCl (2 M, 0.3 mL) was added, the solution stirred at rt for 5 min and then concentrated in vacuo. The residue was dried to give 6-acetamido-1-(6-(1,1-difluoroethyl)pyridin-2-yl)-1H-pyrazolo[4,3-c]pyridine-3-carboxylic acid as a white solid. LCMS m/z=362 [M+H]⁺

Step 2: To a mixture of 6-acetamido-1-(6-(1,1-difluoroethyl)pyridin-2-yl)-1H-pyrazolo[4,3-c]pyridine-3-carboxylic acid (90 mg, 0.249 mmol), azetidine (142 mg, 2.49 mmol) and HATU (161 mg, 0.423 mmol) in DMF (2 mL) was added DIPEA (217 μL, 1.25 mmol) and the reaction mixture was stirred at rt overnight. The mixture was diluted with EtOAc, washed with water (3×), then brine. The organic layer was separated, dried and concentrated. The crude was purified by HPLC to give N-(3-(azetidine-1-carbonyl)-1-(6-(1,1-difluoroethyl)pyridin-2-yl)-1H-pyrazolo[4,3-c]pyridin-6-yl)acetamide (10 mg, 10% yield) as a white powder. LCMS m/z=401 [M+H]^{+ 1}H NMR (500 MHz, DMSO-d₆) δ:10.87 (s, 1H), 9.42 (d, J=0.92 Hz, 1H), 9.23 (d, J=1.22 Hz, 1H), 8.20-8.28 (m, 1H), 8.15 (d, J=8.39 Hz, 1H), 7.73 (dd, J=0.76, 7.63 Hz, 1H), 4.75 (t, J=7.63 Hz, 2H), 4.18 (t, J=7.71 Hz, 2H), 2.38 (td, J=7.67, 15.49 Hz, 2H), 2.22-2.34 (m, 3H), 2.16 (s, 3H).

### Example 450: N-(1-(6-(1,1-difluoroethyl)pyridin-2-yl)-3-(methoxymethyl)-1H-pyrazolo[4,3-c]pyridin-6-yl)acetamide

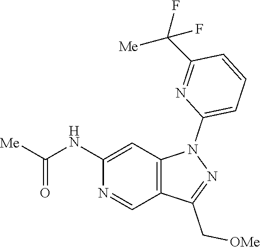 [SMILES: COCc1nn(-c2cccc(C(C)(F)F)n2)c2cc(NC(C)=O)ncc12]

N-(1-(6-(1,1-Difluoroethyl)pyridin-2-yl)-3-(methoxymethyl)-1H-pyrazolo[4,3-c]pyridin-6-yl)acetamide was obtained as a white powder, 13 mg, 20%, from 6-chloro-1-(6-(1,1-difluoroethyl)pyridin-2-yl)-3-(methoxymethyl)-1H-pyrazolo[4,3-c]pyridine (Preparation 708) and acetamide, following a similar procedure to that described in Example 436. LCMS m/z=362 [M+H]⁺ 1H NMR (400 MHz, CDCl₃) 9.82-10.12 (m, 1H), 9.66 (s, 1H), 8.95 (d, J=1.00 Hz, 1H), 8.07-8.14 (m, 1H), 7.97-8.05 (m, 1H), 7.64 (dd, J=1.00, 7.53 Hz, 1H), 4.93 (s, 2H), 3.54 (s, 3H), 2.19-2.40 (m, 6H).

### Example 451: N-(3-(3,3-difluorocyclobutyl)-1-(6-(1,1-difluoroethyl)pyridin-2-yl)-1H-pyrazolo[4,3-c]pyridin-6-yl)acetamide

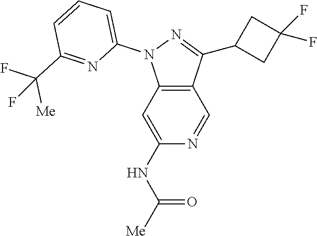 [SMILES: CC(=O)Nc1cc2c(cn1)c(C1CC(F)(F)C1)nn2-c1cccc(C(C)(F)F)n1]

A mixture of acetamide (22.69 mg, 0.384 mmol), 6-chloro-3-(3,3-difluorocyclobutyl)-1-(6-(1,1-difluoroethyl)pyridin-2-yl)-1H-pyrazolo[4,3-c]pyridine (Preparation 705, 73.91 mg, 0.192 mmol), Cs₂CO₃ (125.18 mg, 0.384 mmol) and BrettPhos Pd G3 (34.83 mg, 38.4 μmol) in dioxane (5.5 mL) was sparged with N₂ for 10 min and the reaction was heated at 100° C. for 1 h. The cooled reaction mixture was purified by silica gel chromatography eluting with 3:1 EtOAc, EtOH 2% NH₄OH and the residue was recrystallized from MeOH to give N-(3-(3,3-difluorocyclobutyl)-1-(6-(1,1-difluoroethyl)pyridin-2-yl)-1H-pyrazolo[4,3-c]pyridin-6-yl)acetamide, 26 mg of off-white solid. LCMS m/z=408 [M+H]⁺

### Example 452: N-(1-(6-(1,1-difluoroethyl)pyridin-2-yl)-7-fluoro-1H-pyrazolo[4,3-c]pyridin-6-yl)acetamide

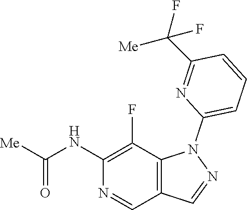 [SMILES: CC(=O)Nc1ncc2cnn(-c3cccc(C(C)(F)F)n3)c2c1F]

A mixture of 6-chloro-1-(6-(1,1-difluoroethyl)pyridin-2-yl)-7-fluoro-1H-pyrazolo[4,3-c]pyridine (Preparation 713, 80 mg, 0.256 mmol), acetamide (30 mg, 0.512 mmol), Cs₂CO₃ (167 mg, 0.512 mmol) and BrettPhos Pd G3 (12 mg, 12.8 μmol) in dioxane (2 mL) was degassed with N₂, then heated at 100° C. in a sealed tube for 30 min. The cooled mixture was filtered and the filtrate purified by column chromatography on silica gel (0-80% EtOAc-EtOH 3:1 with 20% NH₄OH in heptane) to give N-(1-(6-(1,1-difluoroethyl)pyridin-2-yl)-7-fluoro-1H-pyrazolo[4,3-c]pyridin-6-yl)acetamide, 45 mg, 52%) as a white powder. LCMS m/z=336 [M+H]^{+ 1}H NMR (400 MHz, CDCl₃) δ 8.75 (s, 1H), 8.36 (d, J=1.76 Hz, 1H), 8.00-8.07 (m, 1H), 7.94-7.99 (m, 1H), 7.80 (br s, 1H), 7.67-7.72 (m, 1H), 2.39 (s, 3H), 2.12 (t, J=18.82 Hz, 3H)

### Example 453: N-(1-(6-(3-fluoroazetidin-3-yl)pyridin-2-yl)-3-methyl-1H-pyrazolo[4,3-c]pyridin-6-yl)acetamide

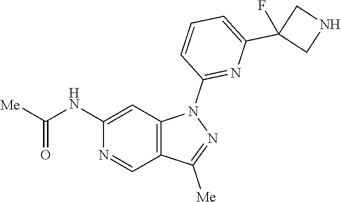 [SMILES: CC(=O)Nc1cc2c(cn1)c(C)nn2-c1cccc(C2(F)CNC2)n1]

Step 1: tert-Butyl 3-(6-(6-acetamido-3-methyl-1H-pyrazolo[4,3-c]pyridin-1-yl)pyridin-2-yl)-3-fluoroazetidine-1-carboxylate was obtained as a white powder, 33 mg, 15%, from tert-butyl 3-(6-(6-chloro-3-methyl-1H-pyrazolo[4,3-c]pyridin-1-yl)pyridin-2-yl)-3-fluoroazetidine-1-carboxylate (Preparation 714) and acetamide, following the procedure described in Example 452.

Step 2: To a solution of tert-Butyl 3-(6-(6-acetamido-3-methyl-1H-pyrazolo[4,3-c]pyridin-1-yl)pyridin-2-yl)-3-fluoroazetidine-1-carboxylate (30 mg, 68.1 μmol) in DCM (1 mL) was added TFA (0.1 mL) and the reaction mixture was stirred at rt overnight. The mixture was concentrated under vacuum, the residue was diluted with EtOAc, and washed with aq. NaHCO₃. The organic layer was separated, dried and concentrated. The crude was purified by HPLC to give N-(1-(6-(3-fluoroazetidin-3-yl)pyridin-2-yl)-3-methyl-1H-pyrazolo[4,3-c]pyridin-6-yl)acetamide (24 mg, 77% yield, TFA salt) as white powder. LCMS m/z=341 [M+H]^{+ 1}H NMR (DMSO-d₆, 500 MHz) δ 10.98 (s, 1H), 9.41 (s, 1H), 8.99 (d, 1H, J=1.1 Hz), 8.15 (dd, 1H, J=7.6, 8.2 Hz), 8.02 (d, 1H, J=8.2 Hz), 7.51 (d, 1H, J=7.6 Hz), 4.9-5.0 (m, 2H), 4.6-4.7 (m, 2H), 2.66 (s, 3H), 2.19 (s, 3H).

### Example 454: N-(1-(6-(1-acetyl-3-fluoroazetidin-3-yl)pyridin-2-yl)-3-methyl-1H-pyrazolo[4,3-c]pyridin-6-yl)acetamide

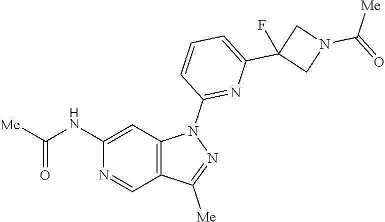 [SMILES: CC(=O)Nc1cc2c(cn1)c(C)nn2-c1cccc(C2(F)CN(C(C)=O)C2)n1]

N-(1-(6-(1-Acetyl-3-fluoroazetidin-3-yl)pyridin-2-yl)-3-methyl-1H-pyrazolo[4,3-c]pyridin-6-yl)acetamide was obtained as a white powder, from 1-(3-(6-(6-chloro-3-methyl-1H-pyrazolo[4,3-c]pyridin-1-yl)pyridin-2-yl)-3-fluoroazetidin-1-yl)ethan-1-one (Preparation 716) and acetamide, following a similar procedure to that described in Example 452. LCMS m/z=383 [M+H]^{+ 1}H NMR (DMSO-d₆, 500 MHz) δ 10.75 (s, 1H), 9.24 (s, 1H), 8.95 (d, 1H, J=0.9 Hz), 8.1-8.2 (m, 1H), 7.97 (d, 1H, J=8.2 Hz), 7.52 (d, 1H, J=7.5 Hz), 5.2-5.3 (m, 1H), 4.6-4.7 (m, 1H), 4.2-4.4 (m, 2H), 2.65 (s, 3H), 2.13 (s, 3H), 1.97 (s, 3H).

### Example 455: N-(1-(6-(3-fluoro-1-(methylsulfonyl)azetidin-3-yl)pyridin-2-yl)-3-methyl-1H-pyrazolo[4,3-c]pyridin-6-yl)acetamide

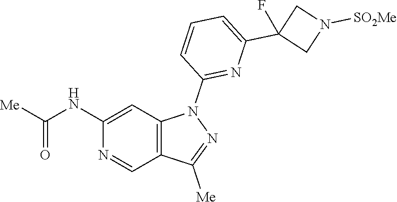 [SMILES: CC(=O)Nc1cc2c(cn1)c(C)nn2-c1cccc(C2(F)CN(S(C)(=O)=O)C2)n1]

N-(1-(6-(3-Fluoro-1-(methylsulfonyl)azetidin-3-yl)pyridin-2-yl)-3-methyl-1H-pyrazolo[4,3-c]pyridin-6-yl)acetamide was prepared from 6-chloro-1-(6-(3-fluoro-1-(methylsulfonyl)azetidin-3-yl)pyridin-2-yl)-3-methyl-1H-pyrazolo[4,3-c]pyridine (Preparation 717) and acetamide, following a similar procedure to that described in Example 452. LCMS m/z=419 [M+H]⁺1H NMR (DMSO-d₆, 500 MHz) δ 10.75 (s, 1H), 9.25 (s, 1H), 8.95 (d, 1H, J=1.1 Hz), 8.14 (t, 1H, J=7.9 Hz), 7.98 (d, 1H, J=8.4 Hz), 7.55 (dd, 1H, J=0.6, 7.5 Hz), 4.7-4.8 (m, 2H), 4.4-4.5 (m, 2H), 3.08 (s, 3H), 2.65 (s, 3H), 2.14 (s, 3H).

### Example 456: N-(1-(6-(1,1-difluoroethyl)pyridin-2-yl)-3-(1-methoxyethyl)-1H-pyrazolo[4,3-c]pyridin-6-yl)acetamide

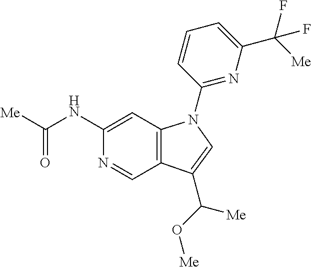 [SMILES: COC(C)c1cn(-c2cccc(C(C)(F)F)n2)c2cc(NC(C)=O)ncc12]

N-(1-(6-(1,1-Difluoroethyl)pyridin-2-yl)-3-(1-methoxyethyl)-1H-pyrazolo[4,3-c]pyridin-6-yl)acetamide was obtained as a white powder, 24 mg, 80% from 6-chloro-1-(6-(1,1-difluoroethyl)pyridin-2-yl)-3-(1-methoxyethyl)-1H-pyrazolo[4,3-c]pyridine (Preparation 712) and acetamide, following a similar procedure to that described in Example 452. LCMS m/z=376 [M+H]^{+ 1}H NMR (400 MHz, MeOD-d₄) δ 9.41 (s, 1H), 8.98 (d, J=1.00 Hz, 1H), 8.06-8.15 (m, 2H), 7.60 (dd, J=1.25, 7.03 Hz, 1H), 4.86-4.92 (m, 1H), 3.39 (s, 3H), 2.20-2.31 (m, 6H), 1.70 (d, J=6.53 Hz, 3H).

### Example 457: N-(3-cyclopropyl-1-(1-isopropyl-6-oxo-1,6-dihydropyridin-3-yl)-1H-pyrrolo[3,2-c]pyridin-6-yl)acetamide

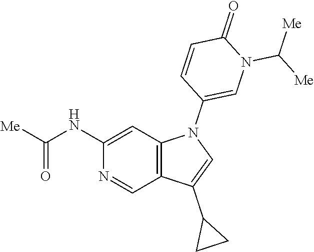 [SMILES: CC(=O)Nc1cc2c(cn1)c(C1CC1)cn2-c1ccc(=O)n(C(C)C)c1]

To a solution of N-(3-cyclopropyl-1H-pyrrolo[3,2-c]pyridin-6-yl)acetamide (Preparation 21, 100 mg, 0.46 mmol) in DMSO (5 mL) was added 5-bromo-1-isopropylpyridin-2(1H)-one (200.8 mg, 0.93 mmol), CuI (17.70 mg, 0.09 mmol), K₂CO₃ (128.41 mg, 0.93 mmol), N,N′-dimethylmethanediamine (8.19 mg, 0.93 mmol) and the reaction mixture was stirred at 100° C. for 16 h under N₂. The mixture was concentrated and purified by Prep-HPLC-C (43% to 73%) to give N-(3-cyclopropyl-1-(1-isopropyl-6-oxo-1,6-dihydropyridin-3-yl)-1H-pyrrolo[3,2-c]pyridin-6-yl)acetamide (38.9 mg, 23.9% yield) as a white solid. LCMS m/z=351.1 [M+H]⁺. ¹H NMR (400 MHz, CDCl₃) δ: ppm 8.66 (s, 1H), 8.31 (s, 1H), 8.16 (s, 1H), 7.52 (d, J=2.4 Hz, 1H), 7.35-7.38 (m, 1H), 6.82 (d, J=1.2 Hz, 1H), 6.68 (d, J=9.6 Hz, 1H), 5.28-5.38 (m, 1H), 2.20 (s, 3H), 1.96-2.01 (m, 1H), 1.43 (d, J=6.8 Hz, 6H), 0.93-1.02 (m, 2H), 0.66-0.74 (m, 2H)

Examples 458 to 462: The compounds in the following table were obtained from N-(3-cyclopropyl-1H-pyrrolo[3,2-c]pyridin-6-yl)acetamide (Preparation 21) and the appropriate aryl bromide, following a similar procedure to that described in Example 457.

| Example No | Name, Structure, Aryl bromide, HPLC, Data |
|---|---|
| 458 | N-(3-cyclopropyl-1-(1-(difluoromethyl)-6-oxo-1,6- |
| | dihydropyridin-3-yl)-1H-pyrrolo[3,2-c]pyridin-6-yl)acetamide |
| | 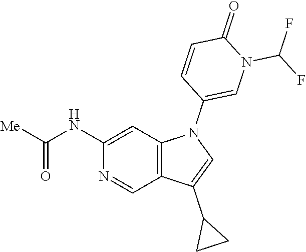 [SMILES: CC(=O)Nc1cc2c(cn1)c(C1CC1)cn2-c1ccc(=O)n(C(F)F)c1] |
| | 5-bromo-1-(difluoromethyl)pyridin-2(1H)-one |
| | Prep-HPLC-C (24% to 53% MeCN). 40.3 mg, 24.2% as yellow |
| | solid. |
| | LCMS m/z = 359.1 [M + H]⁺. ¹H NMR: (400 MHz, CDCl₃) δ ppm: |
| | 8.69 (br s, 1H), 8.21-8.22 (m, 2H), 7.51-7.87 (m, 3H), 6.82 (s, 1H), 6.70 |
| | (d, J = 9.6 Hz, 1H), 2.21 (s, 3H), 1.93-1.98 (m, 1H), 0.97-1.00 (m, 2H), |
| | 0.70-0.71 (m, 2H). |
| 459 | N-(3-cyclopropyl-1-(1-(difluoromethyl)-5-methyl-6-oxo-1,6- |
| | dihydropyridin-3-y1)-1H-pyrrolo[3,2-c]pyridin-6-yl)acetamide |
| | 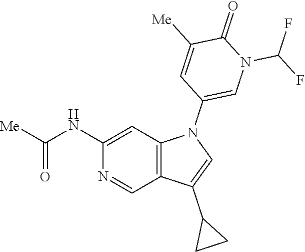 [SMILES: CC(=O)Nc1cc2c(cn1)c(C1CC1)cn2-c1cc(C)c(=O)n(C(F)F)c1] |
| | 5-bromo-1-(difluoromethyl)-3-methylpyridin-2(1H)-one |
| | (Preparation 718) |
| | Prep-HPLC-C (24% to 54% MeCN). 25 mg, 14.5% as a white |
| | solid. LCMS m/z = 373.1 [M + H]⁺ ¹H NMR: (400 MHz, CDCl₃) δ ppm: |
| | 8.67 (br s, 1H), 8.16-8.24 (m, 2H), 7.76 (t, J-60.4 Hz, 1H), 7.49 (s, 1H), |
| | 7.37 (s, 1H), 6.81 (s, 1H), 2.22 (s, 6H), 1.95-1.97 (m, 1H), 0.96-0.98 (m, |
| | 2H), 0.69-0.71 (m, 2H). |
| 460^{A} | N-(3-cyclopropyl-1-(6-(difluoromethoxy)-5-methyl-1,6- |
| | dihydropyridin-3-yl)-1H-pyrrolo[3,2-c]pyridin-6-yl)acetamide |
| | 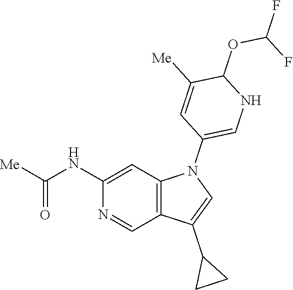 [SMILES: CC(=O)Nc1cc2c(cn1)c(C1CC1)cn2C1=CNC(OC(F)F)C(C)=C1] |
| | 5-bromo-2-(difluoromethoxy)-3-methyl-1,2-dihydropyridine |
| | (Preparation 719) |
| | Prep-HPLC-C (40% to 70% MeCN). 21.1 mg, 12.2% yield as a |
| | white solid. LCMS m/z = 373.1 [M + H]⁺ ¹H NMR: (400 MHz, CDCl₃) δ |
| | ppm: 8.73-8.76 (m, 1H), 8.11-8.26 (m, 3H), 7.64 (s, 1H), 7.51 (t, J = 73.2 |
| | Hz, 1H), 6.90 (s, 1H), 2.36 (s, 3H), 2.20 (s, 3H), 1.95-2.01 (m, 1H), 0.95- |
| | 1.01 (m, 2H), 0.69-0.74 (m, 2H). |
| 461 | N-(3-cyclopropyl-1-(1-isopropyl-6-oxo-1,6-dihydropyridazin-3- |
| | y1)-1H-pyrrolo[3,2-c]pyridin-6-yl)acetamide |
| | 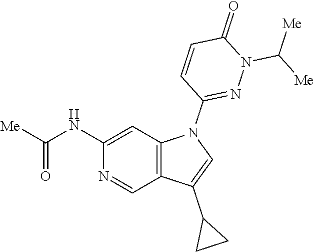 [SMILES: CC(=O)Nc1cc2c(cn1)c(C1CC1)cn2-c1ccc(=O)n(C(C)C)n1] |
| | 6-bromo-2-isopropylpyridazin-3(2H)-one |
| | Prep-HPLC-C (21% to 50% MeCN). 42 mg, 25.7% yield as white |
| | solid. LCMS m/z = 352.1 [M + H]⁺ ¹H NMR: (400 MHz, DMSO-d₆) δ |
| | ppm: 10.46 (s, 1H), 8.94 (s, 1H), 8.71 (s, 1H), 7.95 (d, J= 9.6 Hz, 1H), |
| | 7.63 (s, 1H), 7.16 (d, J = 10.0 Hz, 1H), 5.18-5.26 (m, 1H), 2.11 (s, 3H), |
| | 2.05-2.07 (m, 1H), 1.42 (d, J = 6.8 Hz, 6H), 0.76-0.77 (m, 2H), 0.75-0.76 |
| | (m, 2H). |
| 462 | N-(3-cyclopropyl-1-(4-isopropyl-5-oxo-4,5-dihydropyrazin-2-yl)- |
| | 1H-pyrrolo[3,2-c]pyridin-6-yl)acetamide |
| | 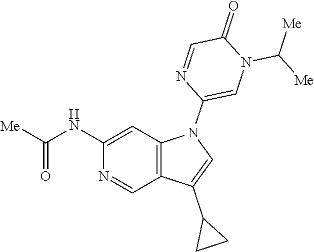 [SMILES: CC(=O)Nc1cc2c(cn1)c(C1CC1)cn2-c1cn(C(C)C)c(=O)cn1] |
| | 5-bromo-1-isopropylpyrazin-2(1H)-one |
| | Prep-HPLC-F (34% to 64% MeCN). 27.2 mg, 16.7% yield as a |
| | white solid. LCMS m/z = 352.1 [M + H]⁺ ¹H NMR: (500 MHz, CDCl₃) δ |
| | ppm: 8.67 (s, 1H), 8.38 (s, 1H), 8.15 (s, 1H), 8.03 (s, 1H), 7.51 (s, 1H), |
| | 7.19 (s, 1H), 5.21-5.26 (m, 1H), 2.22 (s, 3H), 1.95-2.01 (m, 1H), 1.50 (d, |
| | J = 7.0 Hz, 6H), 0.95-0.99 (m, 2H), 0.71-0.74 (m, 2H). |

### Example 463: N-(1-(6-(1,1-difluoroethyl)pyridin-2-yl)-3-(difluoromethyl)-1H-pyrrolo[3,2-c]pyridin-6-yl)acetamide

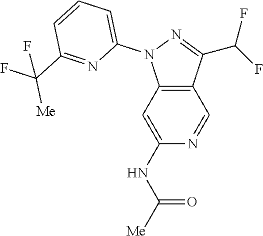 [SMILES: CC(=O)Nc1cc2c(cn1)c(C(F)F)nn2-c1cccc(C(C)(F)F)n1]

A mixture of acetamide (16.50 mg, 0.279 mmol), 6-chloro-1-(6-(1,1-difluoroethyl)pyridin-2-yl)-3-(difluoromethyl)-1H-pyrrolo[3,2-c]pyridine (Preparation 723, 48 mg, 0.14 mmol), Cs₂CO₃ (91.0 mg, 0.279 mmol) and BrettPhos Pd G3 (25.32 mg, 27.9 μmol) in dioxane (4 mL) was sparged with N₂ for 10 min and the reaction was heated at 100° C. for 2 h. The cooled reaction was purified directly by silica gel chromatography (heptane to 3:1 EtOAc:EtOH 2% NH₄OH) to give N-(1-(6-(1,1-difluoroethyl)pyridin-2-yl)-3-(difluoromethyl)-1H-pyrazolo[4,3-c]pyridin-6-yl)acetamide, 21 mg as a brown solid. LCMS m/z=367 [M+H]⁺.

### Example 464: N-(1-(4-(1,1-difluoroethyl)pyrimidin-2-yl)-3-(4-methylpiperazin-1-yl)-1H-pyrrolo[3,2-c]pyridin-6-yl)acetamide

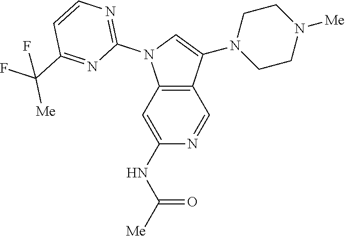 [SMILES: CC(=O)Nc1cc2c(cn1)c(N1CCN(C)CC1)cn2-c1nccc(C(C)(F)F)n1]

A mixture of 6-chloro-1-(4-(1,1-difluoroethyl)pyrimidin-2-yl)-3-(4-methylpiperazin-1-yl)-1H-pyrrolo[3,2-c]pyridine (Preparation 725, 90 mg, 0.229 mmol), acetamide (68 mg, 1.15 mmol), Cs₂CO₃ (149 mg, 0.458 mmol) and Brettphos Pd G3 (10 mg, 11.5 μmol) in dioxane (2 mL) was degassed with N₂, then heated at 110° C. in a sealed tube for 1 h. The cooled mixture was filtered, and the crude purified by HPLC to give the N-(1-(4-(1,1-difluoroethyl)pyrimidin-2-yl)-3-(4-methylpiperazin-1-yl)-1H-pyrrolo[3,2-c]pyridin-6-yl)acetamide (49 mg, 28% yield). LCMS m/z=416 [M+H]^{+ 1}H NMR (MeOD-d₄, 400 MHz) δ 9.07 (d, 1H, J=5.0 Hz), 8.93 (s, 1H), 8.84 (s, 1H), 8.19 (s, 1H), 7.68 (d, 1H, J=5.0 Hz), 3.81 (br d, 2H, J=12.5 Hz), 3.70 (br d, 2H, J=10.5 Hz), 3.4-3.5 (m, 2H), 3.2-3.3 (m, 2H), 3.04 (s, 3H), 2.34 (s, 3H), 2.1-2.2 (m, 3H).

### Example 465: N-(1-(2-(1,1-difluoroethyl)-6-(pyrimidin-5-yloxy)pyrimidin-4-yl)-1H-pyrrolo[3,2-c]pyridin-6-yl)acetamide trifluoroacetate

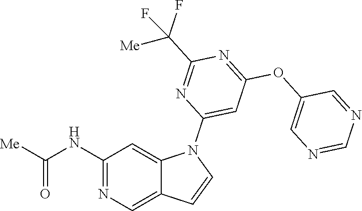 [SMILES: CC(=O)Nc1cc2c(ccn2-c2cc(Oc3cncnc3)nc(C(C)(F)F)n2)cn1]

A mixture of N-(1-(6-chloro-2-(1,1-difluoroethyl)pyrimidin-4-yl)-1H-pyrrolo[3,2-c]pyridin-6-yl)acetamide (Preparation 727, 21.1 mg, 0.060 mmol), pyrimidin-5-ol (6.3 mg, 0.066 mmol) and K₂CO₃ (24.9 mg, 0.180 mmol) in DMF (1 mL) was heated at 35° C. for 16 h in a sealed vessel. After cooling to rt, the reaction mixture was diluted with EtOAc, washed with H₂O (3 mL), and extracted with EtOAc (5 mL, 2×). The combined organic phases were dried over anhydrous MgSO₄ and concentrated in vacuo. The residue was purified by Prep-HPLC-V (5% to 40% MeCN) to give N-(1-(2-(1,1-difluoroethyl)-6-(pyrimidin-5-yloxy)pyrimidin-4-yl)-1H-pyrrolo[3,2-c]pyridin-6-yl)acetamide trifluoroacetate (2.4 mg, yield: 9.7%). LCMS m/z=412.2 [M+H]⁺

### Example 466: N-(1-(2-(1,1-difluoroethyl)-6-(propylamino)pyrimidin-4-yl)-3-ethyl-1H-pyrrolo[3,2-c]pyridin-6-yl)acetamide

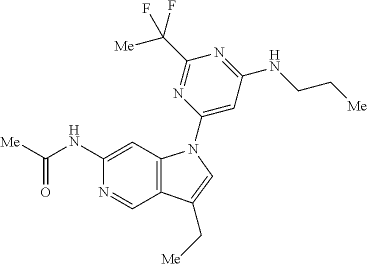 [SMILES: CCCNc1cc(-n2cc(CC)c3cnc(NC(C)=O)cc32)nc(C(C)(F)F)n1]

A mixture of N-(1-(6-chloro-2-(1,1-difluoroethyl)pyrimidin-4-yl)-3-ethyl-1H-pyrrolo[3,2-c]pyridin-6-yl)acetamide (Preparation X, 60 mg, 0.158 mmol), propan-1-amine (9.34 mg, 0.158 mmol) and K₂CO₃ (65.50 mg, 0.474 mmol) in DMF (1.98 g, 27.12 mmol) was stirred at 60° C. for 12 h. The mixture was diluted with EtOAc, washed with aq. NaHCO₃, H₂O and brine. The organic layer was dried over Na₂SO₄ and the solvent removed in vacuo. The crude mixture was purified by HPLC to give N-(1-(6-amino-2-(1,1-difluoroethyl)pyrimidin-4-yl)-1H-pyrrolo[3,2-c]pyridin-6-yl)acetamide, 12.1 mg as a white solid. LCMS/z=403 [M+H]⁺

### Example 467: N-(1-(2-(1,1-difluoroethyl)-6-(1-methyl-1H-pyrazol-3-yl)pyrimidin-4-yl)-3-ethyl-1H-pyrrolo[3,2-c]pyridin-6-yl)acetamide

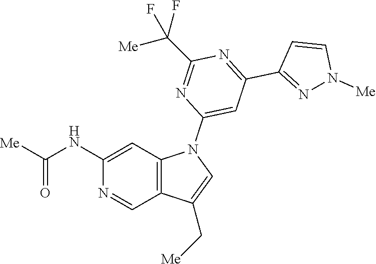 [SMILES: CCc1cn(-c2cc(-c3ccn(C)n3)nc(C(C)(F)F)n2)c2cc(NC(C)=O)ncc12]

A mixture of N-(1-(6-chloro-2-(1,1-difluoroethyl)pyrimidin-4-yl)-3-ethyl-1H-pyrrolo[3,2-c]pyridin-6-yl)acetamide (Preparation 726, 50 mg, 0.132 mmol), 1-methyl-3-(4,4,5,5-tetramethyl-1,3,2-dioxaborolan-2-yl)pyrazole (54.78 mg, 0.263 mmol), K₂CO₃ (36.39 mg, 0.263 mmol) and Pd(dppf)Cl₂ DCM (10.75 mg, 13.17 μmol) in dioxane (1.01 mL) and water (0.2 mL) was degassed under an atmosphere of N₂. The reaction mixture was stirred at 80° C. for 12 h. The mixture was diluted with EtOAc, washed with NaHCO₃, H₂O, and brine and the organic layer was dried over Na₂SO₄ and solvent removed in vacuo. The crude mixture was purified by HPLC to give N-(1-(2-(1,1-difluoroethyl)-6-(1-methyl-1H-pyrazol-3-yl)pyrimidin-4-yl)-3-ethyl-1H-pyrrolo[3,2-c]pyridin-6-yl)acetamide, 43.5 mg, as a solid. LCMS m/z=426 [M+H]⁺

### Example 468: N-(3-(2-cyanocyclopropyl)-1-(4-(1,1-difluoroethyl)pyrimidin-2-yl)-1H-pyrrolo[3,2-c]pyridin-6-yl)acetamide

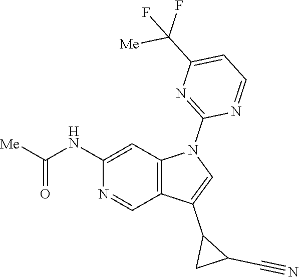 [SMILES: CC(=O)Nc1cc2c(cn1)c(C1CC1C#N)cn2-c1nccc(C(C)(F)F)n1]

To a solution of N-(3-bromo-1-(4-(1,1-difluoroethyl)pyrimidin-2-yl)-1H-pyrrolo[3,2-c]pyridin-6-yl)acetamide (Preparation 386, 100 mg, 0.25 mmol) in dioxane (5 mL) and H₂O (0.5 mL) was added 2-(4,4,5,5-tetramethyl-1,3,2-dioxaborolan-2-yl)cyclopropane-1-carbonitrile (Preparation 375, 365.5 mg, 1.89 mmol), K₂CO₃ (104.7 mg, 0.76 mmol) and Pd(dppf)Cl₂ (16.45 mg, 0.025 mmol) and the mixture was stirred at 80° C. for 16 h under N₂. The mixture was concentrated and purified by Prep-HPLC-L (45% to 75% MeCN) to give N-(3-(2-cyanocyclopropyl)-1-(4-(1,1-difluoroethyl)pyrimidin-2-yl)-1H-pyrrolo[3,2-c]pyridin-6-yl)acetamide (9 mg, 9.33% yield) as a white solid. LCMS m/z=383.0 [M+H]⁺. ¹H NMR (500 MHz, CDCl₃) δ ppm 9.47 (s, 1H), 8.87 (d, J=5.0 Hz, 1H), 8.64 (s, 1H), 8.06 (s, 1H), 7.98 (s, 1H), 7.45 (d, J=5.0 Hz, 1H), 2.76-2.70 (m, 1H), 2.28-2.19 (m, 6H), 1.79-1.58 (m, 3H)

### Example 469: N-(1-(2-(1,1-difluoroethyl)-6-ethylpyrimidin-4-yl)-3-(morpholinomethyl)-1H-pyrrolo[3,2-c]pyridin-6-yl)acetamide

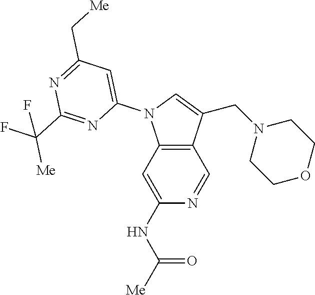 [SMILES: CCc1cc(-n2cc(CN3CCOCC3)c3cnc(NC(C)=O)cc32)nc(C(C)(F)F)n1]

To a suspension of morpholine (25.67 mg, 0.295 mmol) and N-(1-(2-(1,1-difluoroethyl)-6-ethylpyrimidin-4-yl)-3-formyl-1H-pyrrolo[3,2-c]pyridin-6-yl)acetamide (Preparation 724, 110 mg, 0.295 mmol) in MeOH (1 mL) was added NaCNBH₃ (1 M, 1.47 mL) solution in THF and the resulting mixture was stirred at rt for 3 h. The reaction was quenched with saturated NaHCO₃ solution and extracted with DCM (3×). The combined organic layers were washed with brine, dried (MgSO₄), filtered and concentrated in vacuo. The residue was purified by silica gel chromatography (0-10% MeOH in DCM) to give N-(1-(2-(1,1-difluoroethyl)-6-ethylpyrimidin-4-yl)-3-(morpholinomethyl)-1H-pyrrolo[3,2-c]pyridin-6-yl)acetamide (70 mg, 52.4% yield) as a pale yellow solid. LCMS m/z=445.2 [M+H]1H NMR (500 MHz, CDCl₃) δ ppm 9.24 (s, 1H), 8.82 (s, 1H), 8.68 (br s, 1H), 7.74 (s, 1H), 7.36 (s, 1H), 3.71 (br s, 6H), 2.96 (q, J=7.6 Hz, 2H), 2.52 (br s, 3H), 2.17-2.30 (m, 6H), 1.42 (t, J=7.6 Hz, 3H).

### Example 470: N-(1-(2-(1,1-difluoroethyl)-6-ethylpyrimidin-4-yl)-3-((dimethylamino)methyl)-1H-pyrrolo[3,2-c]pyridin-6-yl)acetamide

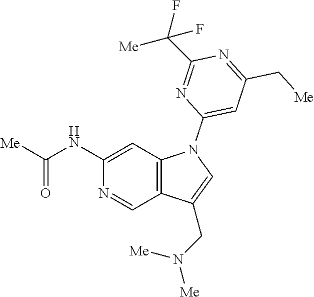 [SMILES: CCc1cc(-n2cc(CN(C)C)c3cnc(NC(C)=O)cc32)nc(C(C)(F)F)n1]

N-(1-(2-(1,1-Difluoroethyl)-6-ethylpyrimidin-4-yl)-3-((dimethylamino)methyl)-1H-pyrrolo[3,2-c]pyridin-6-yl)acetamide was obtained as a pale yellow solid, 50 mg, 40%, from N-methylmethanamine and N-(1-(2-(1,1-difluoroethyl)-6-ethylpyrimidin-4-yl)-3-formyl-1H-pyrrolo[3,2-c]pyridin-6-yl)acetamide (Preparation 724) following the procedure described in Example 469. LCMS m/z=403.2 [M+H]⁺1H NMR (500 MHz, CDCl₃) δ ppm 9.26 (s, 1H), 8.72 (s, 1H), 8.52 (br s, 1H), 7.74 (s, 1H), 7.36 (s, 1H), 3.65 (s, 2H), 2.96 (q, J=7.4 Hz, 2H), 2.33 (s, 6H), 2.18-2.30 (m, 6H), 1.42 (t, J=7.5 Hz, 3H).

### Examples 471 and 472: N-(3-((3-cyano-3-methylpyrrolidin-1-yl)methyl)-1-(2-(1,1-difluoroethyl)-6-ethylpyrimidin-4-yl)-1H-pyrrolo[3,2-c]pyridin-6-yl)acetamide and N-(1-(2-(1,1-difluoroethyl)-6-ethylpyrimidin-4-yl)-3-(hydroxymethyl)-1H-pyrrolo[3,2-c]pyridin-6-yl)acetamide

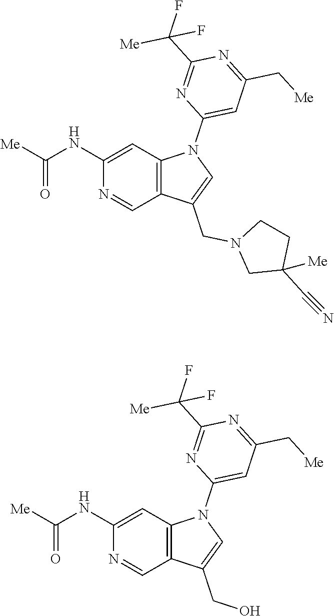 [SMILES: CCc1cc(-n2cc(CN3CCC(C)(C#N)C3)c3cnc(NC(C)=O)cc32)nc(C(C)(F)F)n1 ; CCc1cc(-n2cc(CO)c3cnc(NC(C)=O)cc32)nc(C(C)(F)F)n1]

To a suspension of (3R)-3-methylpyrrolidine-3-carbonitrile (35.70 mg, 0.324 mmol) and N-(1-(2-(1,1-difluoroethyl)-6-ethylpyrimidin-4-yl)-3-formyl-1H-pyrrolo[3,2-c]pyridin-6-yl)acetamide (Preparation 724, 110 mg, 0.295 mmol) in MeOH (1 mL) was added NaCNBH₃ (1 M, 1.47 mL) solution in THE and the resulting mixture was stirred at rt for 3 h. The reaction was quenched with saturated NaHCO₃ solution and extracted with DCM (3×). The combined organic layers were washed with brine, dried (MgSO₄), filtered and concentrated in vacuum. The residue was purified by silica gel chromatography (0-10% MeOH in DCM) to give N-(3-((3-cyano-3-methylpyrrolidin-1-yl)methyl)-1-(2-(1,1-difluoroethyl)-6-ethylpyrimidin-4-yl)-1H-pyrrolo[3,2-c]pyridin-6-yl)acetamide (34.7 mg, 24.7% yield) as a pale yellow solid. LCMS m/z=468.2 [M+H]⁺1H NMR (500 MHz, CDCl₃) δ ppm 9.23 (s, 1H), 8.77 (s, 1H), 8.54 (br s, 1H), 7.75 (s, 1H), 7.37 (s, 1H), 3.87 (s, 2H), 3.05 (d, J=9.4 Hz, 1H), 2.97 (q, J=7.6 Hz, 2H), 2.69-2.87 (m, 2H), 2.57 (d, J=9.4 Hz, 1H), 2.40-2.50 (m, 1H), 2.17-2.30 (m, 6H), 1.81-1.94 (m, 1H), 1.48 (s, 3H), 1.42 (t, J=7.6 Hz, 3H). N-(1-(2-(1,1-difluoroethyl)-6-ethylpyrimidin-4-yl)-3-(hydroxymethyl)-1H-pyrrolo[3,2-c]pyridin-6-yl)acetamide (15.3 mg, 13.7% yield) was obtained as a pale yellow solid. LCMS m/z=376.1 [M+H]⁺

1H NMR (500 MHz, CDCl₃) δ ppm 9.18 (s, 1H), 8.62 (s, 1H), 8.38 (br s, 1H), 7.76 (s, 1H), 7.27-7.29 (m, 1H), 4.92 (s, 2H), 2.94 (q, J=7.6 Hz, 2H), 2.25 (s, 3H), 2.16-2.23 (m, 3H), 1.40 (t, J=7.6 Hz, 3H).

### Example 473: N-(1-(6-(3-methoxytetrahydrofuran-3-yl)pyridin-2-yl)-1H-imidazo[4,5-c]pyridin-6-yl)acetamide

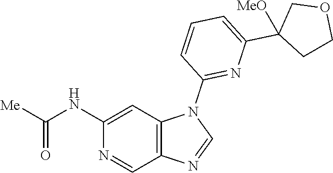 [SMILES: COC1(c2cccc(-n3cnc4cnc(NC(C)=O)cc43)n2)CCOC1]

A mixture of N-(1H-imidazo[4,5-c]pyridin-6-yl)acetamide (Preparation 730, 60 mg, 0.341 mmol), 2-chloro-6-(3-methoxytetrahydrofuran-3-yl)pyridine (87.32 mg, 0.409 mmol) and K₂CO₃ (94.14 mg, 0.681 mmol) in DMSO (2.00 mL) was heated at 150° C. for 2 h. The cooled mixture was diluted with EtOAc, washed with water (3×), then brine. The organic layer was dried and concentrated. The crude was purified by HPLC to give N-(1-(6-(3-methoxytetrahydrofuran-3-yl)pyridin-2-yl)-1H-imidazo[4,5-c]pyridin-6-yl)acetamide (16 mg). LCMS m/z=354 [M+H]⁺

### Example 474: N-(3-(6-oxa-3-azabicyclo[3.1.1]heptan-3-yl)-1-(6-ethyl-2-(2-fluoropropan-2-yl)pyrimidin-4-yl)-1H-pyrazolo[4,3-c]pyridin-6-yl)acetamide

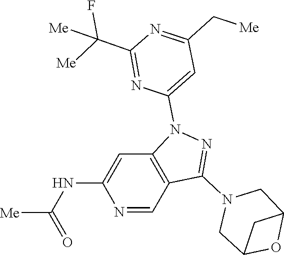 [SMILES: CCc1cc(-n2nc(N3CC4CC(C3)O4)c3cnc(NC(C)=O)cc32)nc(C(C)(C)F)n1]

To a mixture of N-(3-(6-oxa-3-azabicyclo[3.1.1]heptan-3-yl)-1H-pyrazolo[4,3-c]pyridin-6-yl)acetamide (Preparation 543, 109.32 mg, 0.4 mmol), 4-chloro-6-ethyl-2-(2-fluoropropan-2-yl)pyrimidine (Preparation 412, 102 mg, 0.50 mmol) and Cs₂CO₃ (261 mg, 0.8 mmol) in a microwave tube was added DMF (2 mL) and the reaction was heated at 100° C. for 2.5 h. The cooled mixture was partitioned between EtOAc and water and the layers separated. The aqueous layer was extracted with EtOAc. The combined organic phases were washed with brine, water (×2), dried over MgSO₄, filtered, and concentrated. The residue was triturated with MeCN to get N-(3-(6-oxa-3-azabicyclo[3.1.1]heptan-3-yl)-1-(6-ethyl-2-(2-fluoropropan-2-yl)pyrimidin-4-yl)-1H-pyrazolo[4,3-c]pyridin-6-yl)acetamide (96 mg, 54.600 yield) as a light brown solid. LCMS m/z=440.2 [M+H]⁺. ¹H NMR (MeOD-d₄, 400 MHz) δ ppm: 9.46 (s, 1H), 8.99 (d, J=1.0 Hz, 1H), 7.64 (s, 1H), 4.83-4.86 (m, 2H), 4.00-4.12 (m, 4H), 3.35-3.43 (m, 1H), 2.83-2.93 (m, 2H), 2.25 (s, 3H), 2.16 (d, J=8.5 Hz, 1H), 1.87-1.99 (m, 6H), 1.37 (t, J=7.8 Hz, 3H).

| Example | | |
|---|---|---|
| no | Structure, Name | Data |
| 475 | 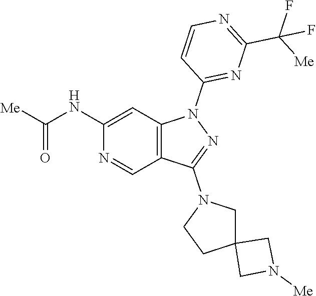 [SMILES: CC(=O)Nc1cc2c(cn1)c(N1CCC3(CN(C)C3)C1)nn2-c1ccnc(C(C)(F)F)n1] | LCMS m/z = 443.2 [M + H]⁺ 1H NMR (500 MHz, MeOD-d₄) ppm 9.35 (br s, 1H), 8.85 (s, 1H), 8.72 (d, J = 5.8 Hz, 1H), 7.81 (br d, J = 5.0 Hz, 1H), 3.85 (s, 2H), 3.76 (br t, J = 6.5 Hz, 2H), 3.42 (s, 4H), 2.44 (s, 3H), 2.26-2.32 (m, 3H), 2.14-2.26 (m, 5H). |
| | N-(1-(2-(1,1-difluoroethyl)pyrimidin-4- | |
| | yl)-3-(2-methyl-2,6-diazaspiro[3.4]octan- | |
| | 6-y1)-1H-pyrazolo[4,3-c]pyridin-6- | |
| | yl)acetamide | |
| 476 | 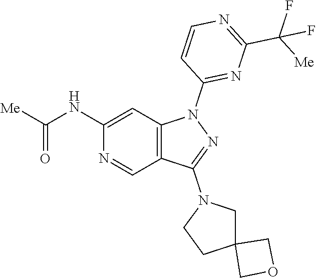 [SMILES: CC(=O)Nc1cc2c(cn1)c(N1CCC3(COC3)C1)nn2-c1ccnc(C(C)(F)F)n1] | LCMS m/z = 430.2 [M + H]⁺ 1H NMR (500 MHz, CDCl₃) ppm 9.66 (s, 1H), 9.34-9.58 (bs, 1H), 8.75-8.88 (m, 2H), 7.81-7.87 (m, 1H), 4.81 (d, J = 6.3 Hz, 2H), 4.74 (d, J = 6.3 Hz, 2H), 4.00 (s, 2H), 3.76-3.86 (m, 2H), 2.42- 2.51 (m, 2H), 2.34 (s, 3H), 2.29 (t, J = 18.8 Hz, 3H). |
| | N-(1-(2-(1,1-difluoroethyl)pyrimidin-4- | |
| | yl)-3-(2-oxa-6-azaspiro[3.4]octan-6-yl)- | |
| | 1H-pyrazolo[4,3-c]pyridin-6-yl)acetamide | |
| 477 | 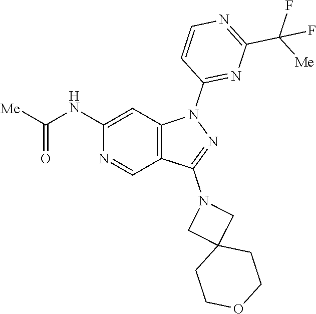 [SMILES: CC(=O)Nc1cc2c(cn1)c(N1CC3(CCOCC3)C1)nn2-c1ccnc(C(C)(F)F)n1] | LCMS m/z = 444.4 [M + H]⁺ 1H NMR (500 MHz, CDCl₃) ppm 9.63 (s, 1H), 9.45 (br s, 1H), 8.81 (d, J = 5.6 Hz, 1H), 8.51-8.65 (m, 1H), 7.79 (d, J = 5.6 Hz, 1H), 4.11 (s, 4H), 3.62-3.81 (m, 4H), 2.33 (s, 3H), 2.27 (t, J = 18.8 Hz, 3H), 1.95 (br t, J = 5.0 Hz, 4H). |
| | N-(1-(2-(1,1-difluoroethyl)pyrimidin-4- | |
| | yl)-3-(7-oxa-2-azaspiro[3.5]nonan-2-yl)- | |
| | 1H-pyrazolo[4,3-c]pyridin-6-yl)acetamide | |
| 478 | 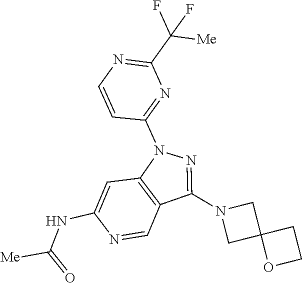 [SMILES: CC(=O)Nc1cc2c(cn1)c(N1CC3(CCO3)C1)nn2-c1ccnc(C(C)(F)F)n1] | LCMS m/z = 416.4 [M + H]⁺ 1H NMR (500 MHz, CDCl₃) ppm 9.44-9.75 (m, 2H), 8.82 (br d, J = 5.3 Hz, 1H), 8.53 (s, 1H), 7.74- 7.88 (m, 1H), 4.63 (br t, J = 7.3 Hz, 2H), 4.52 (s, 4H), 3.02 (br t, J = 7.3 Hz, 2H), 2.33 (s, 3H), 2.27 (t, J = 20.0 Hz, 3H). |
| | N-(1-(2-(1,1-difluoroethyl)pyrimidin-4- | |
| | yl)-3-(1-oxa-6-azaspiro[3.3]heptan-6-y1)- | |
| | 1H-pyrazolo[4,3-c]pyridin-6-yl)acetamide | |
| 479 | 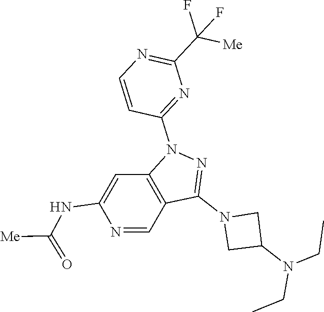 [SMILES: CCN(CC)C1CN(c2nn(-c3ccnc(C(C)(F)F)n3)c3cc(NC(C)=O)ncc23)C1] | LCMS m/z = 445.4 [M + H]⁺ 1H NMR (500 MHz, CDCl₃) ppm 9.43 (br s, 1H), 8.74 (d, J = 5.8 Hz, 1H), 8.39-8.57 (m, 1H), 8.24 (br s, 1H), 7.73 (br d, J = 5.5 Hz, 1H), 4.30-4.45 (m, 2H), 4.23-4.62 (m, 1H), 3.84-4.14 (m, 1H), 2.49- 3.03 (m, 4H), 2.18-2.38 (m, 6H), 0.99-1.38 (m, 6H). |
| | N-(3-(3-(diethylamino)azetidin-1-yl)-1-(2- | |
| | (1,1-difluoroethyl)pyrimidin-4-y1)-1H- | |
| | pyrazolo[4,3-c]pyridin-6-yl)acetamide | |
| 480 | 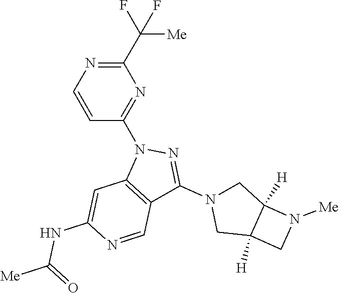 [SMILES: CC(=O)Nc1cc2c(cn1)c(N1C[C@@H]3CN(C)[C@@H]3C1)nn2-c1ccnc(C(C)(F)F)n1] | LCMS m/z = 429.4 [M + H]⁺ |
| | N-(1-(2-(1,1-difluoroethyl)pyrimidin-4- | |
| | yl)-3-((1S,5S)-6-methyl-3,6- | |
| | diazabicyclo[3.2.0]heptan-3-yl)-1H- | |
| | pyrazolo[4,3-c]pyridin-6-yl)acetamide | |
| 481 | 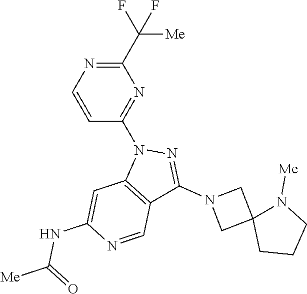 [SMILES: CC(=O)Nc1cc2c(cn1)c(N1CC3(CCCN3C)C1)nn2-c1ccnc(C(C)(F)F)n1] | LCMS m/z = 443.4 [M + H]⁺ |
| | N-(1-(2-(1,1-difluoroethyl)pyrimidin-4- | |
| | yl)-3-(5-methyl-2,5-diazaspiro[3.4]octan- | |
| | 2-y1)-1H-pyrazolo[4,3-c]pyridin-6- | |
| | yl)acetamide | |
| 482 | 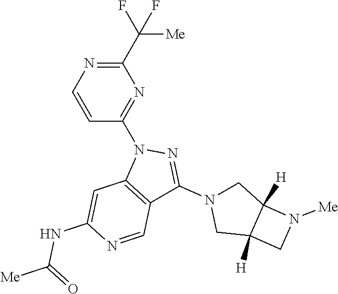 [SMILES: CC(=O)Nc1cc2c(cn1)c(N1C[C@H]3CN(C)[C@H]3C1)nn2-c1ccnc(C(C)(F)F)n1] | LCMS m/z = 429.2 [M + H]⁺ |
| | N-(1-(2-(1,1-difluoroethyl)pyrimidin-4- | |
| | yl)-3-((1R,5R)-6-methyl-3,6- | |
| | diazabicyclo[3.2.0]heptan-3-y1)-1H- | |
| | pyrazolo[4,3-c]pyridin-6-yl)acetamide | |
| 483 | 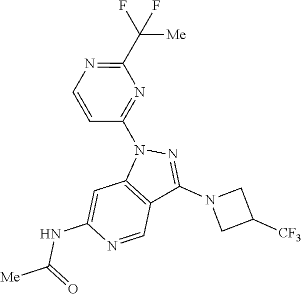 [SMILES: CC(=O)Nc1cc2c(cn1)c(N1CC(C(F)(F)F)C1)nn2-c1ccnc(C(C)(F)F)n1] | LCMS m/z = 442.3 [M + H]⁺ 1H NMR (500 MHz, CDCl₃) ppm 9.54-9.78 (m, 2H), 8.77 (d, J = 5.6 Hz, 1H), 8.56 (br s, 1H), 7.74 (d, J = 5.6 Hz, 1H), 4.41-4.51 (m, 2H), 4.29-4.41 (m, 2H), 3.47- 3.63 (m, 1H), 2.27 (s, 3H), 2.14- 2.25 (m, 3H). |
| | N-(1-(2-(1,1-difluoroethyl)pyrimidin-4- | |
| | yl)-3-(3-(trifluoromethyl)azetidin-1-yl)- | |
| | 1H-pyrazolo[4,3-c]pyridin-6-yl)acetamide | |
| 484 | 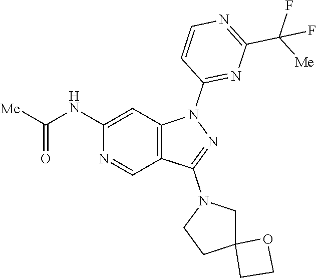 [SMILES: CC(=O)Nc1cc2c(cn1)c(N1CCC3(CCO3)C1)nn2-c1ccnc(C(C)(F)F)n1] | LCMS m/z = 430.3 [M + H]⁺ 1H NMR (500 MHz, CDCl₃) ppm 9.52-9.71 (m, 2H), 8.80 (d, J = 5.8 Hz, 1H), 8.74 (s, 1H), 7.82 (d, J = 5.6 Hz, 1H), 4.54-4.69 (m, 2H), 4.15 (d, J = 11.0 Hz, 1H), 3.74- 3.91 (m, 3H), 2.90 (dt, J = 11.5, 7.8 Hz, 1H), 2.75-2.86 (m, 1H), 2.57-2.69 (m, 1H), 2.31-2.37 (m, 3H), 2.18-2.31 (m, 4H). |
| | N-(1-(2-(1,1-difluoroethyl)pyrimidin-4- | |
| | yl)-3-(1-oxa-6-azaspiro[3.4]octan-6-yl)- | |
| | 1H-pyrazolo[4,3-c]pyridin-6-yl)acetamide | |
| 485 | 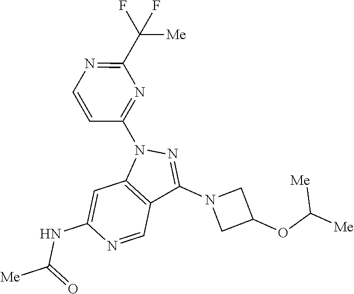 [SMILES: CC(=O)Nc1cc2c(cn1)c(N1CC(OC(C)C)C1)nn2-c1ccnc(C(C)(F)F)n1] | LCMS m/z = 432.4 [M + H]⁺ 1H NMR (500 MHz, CDCl₃) ppm 9.56 (br s, 1H), 8.86 (br s, 1H), 8.80 (d, J = 5.6 Hz, 1H), 8.53 (s, 1H), 7.80 (d, J = 5.6 Hz, 1H), 4.66 (quin, J = 5.6 Hz, 1H), 4.54 (t, J = 7.4 Hz, 2H), 4.21 (dd, J = 8.3, 5.0 Hz, 2H), 3.65-3.78 (m, 1H), 2.23- 2.37 (m, 6H), 1.23 (d, J = 6.1 Hz, 6H). |
| | N-(1-(2-(1,1-difluoroethyl)pyrimidin-4- | |
| | yl)-3-(3-isopropoxyazetidin-1-y1)-1H- | |
| | pyrazolo[4,3-c]pyridin-6-yl)acetamide | |
| 486 | 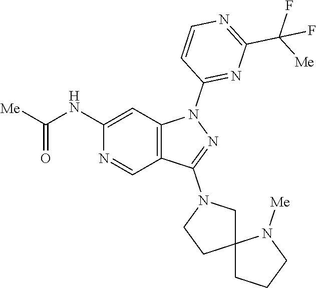 [SMILES: CC(=O)Nc1cc2c(cn1)c(N1CCC3(CCCN3C)C1)nn2-c1ccnc(C(C)(F)F)n1] | LCMS m/z = 457.4 [M + H]⁺ |
| | N-(1-(2-(1,1-difluoroethyl)pyrimidin-4- | |
| | y1)-3-(1-methyl-1,7-diazaspiro[4.4]nonan- | |
| | 7-yl)-1H-pyrazolo[4,3-c]pyridin-6- | |
| | yl)acetamide | |
| 487 | 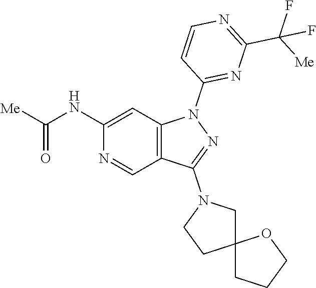 [SMILES: CC(=O)Nc1cc2c(cn1)c(N1CCC3(CCCO3)C1)nn2-c1ccnc(C(C)(F)F)n1] | LCMS m/z = 444.4 [M + H]⁺ 1H NMR (500 MHz, CDCl₃) ppm 9.52-9.71 (m, 2H), 8.80 (d, J = 5.8 Hz, 1H), 8.74 (s, 1H), 7.82 (d, J = 5.6 Hz, 1H), 4.54-4.69 (m, 2H), 4.15 (d, J = 11.0 Hz, 1H), 3.74- 3.91 (m, 3H), 2.90 (dt, J = 11.5, 7.8 Hz, 1H), 2.75-2.86 (m, 1H), 2.57-2.69 (m, 1H), 2.31-2.37 (m, 3H), 2.18-2.31 (m, 4H). |
| | N-(1-(2-(1,1-difluoroethyl)pyrimidin-4- | |
| | yl)-3-(1-oxa-7-azaspiro[4.4]nonan-7-yl)- | |
| | 1H-pyrazolo[4,3-c]pyridin-6-yl)acetamide | |
| 488 | 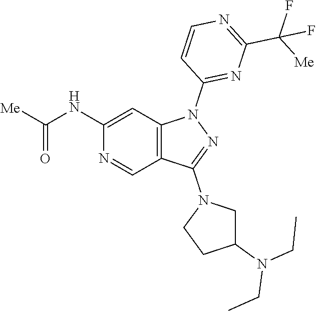 [SMILES: CCN(CC)C1CCN(c2nn(-c3ccnc(C(C)(F)F)n3)c3cc(NC(C)=O)ncc23)C1] | LCMS m/z = 459.2 [M + H]⁺ |
| | N-(3-(3-(diethylamino)pyrrolidin-1-yl)-1- | |
| | (2-(1,1-difluoroethyl)pyrimidin-4-y1)-1H- | |
| | pyrazolo[4,3-c]pyridin-6-yl)acetamide | |
| 489 | 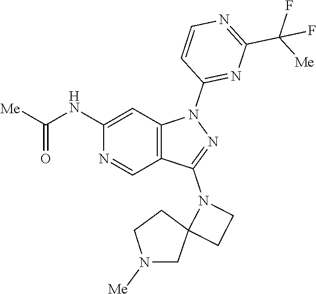 [SMILES: CC(=O)Nc1cc2c(cn1)c(N1CCC13CCN(C)C3)nn2-c1ccnc(C(C)(F)F)n1] | LCMS m/z = 443.1 [M + H]⁺ |
| | N-(1-(2-(1,1-difluoroethyl)pyrimidin-4- | |
| | yl)-3-(6-methyl-1,6-diazaspiro[3.4]octan- | |
| | 1-yl)-1H-pyrazolo[4,3-c]pyridin-6- | |
| | yl)acetamide | |
| 490 | 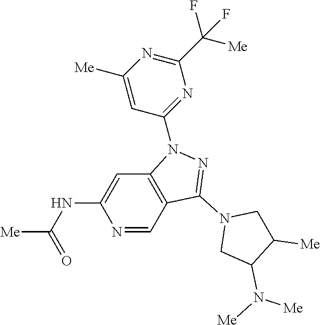 [SMILES: CC(=O)Nc1cc2c(cn1)c(N1CC(C)C(N(C)C)C1)nn2-c1cc(C)nc(C(C)(F)F)n1] | LCMS m/z = 459.1 [M + H]⁺ 1H NMR (400 MHz, CDCl₃) δ ppm 9.33-9.65 (bs, 1H), 8.59- 8.95 (bs, 1H), 8.03-8.25 (bs, 1H), 7.63 (s, 1H), 4.03-4.19 (m, 1H), 3.81-4.03 (m, 2H), 3.33-3.46 (m, 1H), 3.10-3.32 (m, 1H), 2.65 (s, 3H), 2.53-2.64 (m, 4H), 2.21- 2.36 (m, 6H), 1.31 (d, J = 8 Hz, 3H) |
| | N-(1-(2-(1,1-difluoroethyl)-6- | |
| | methylpyrimidin-4-y1)-3-(3- | |
| | (dimethylamino)-4-methylpyrrolidin-1- | |
| | yl)-1H-pyrazolo[4,3-c]pyridin-6- | |
| | yl)acetamide | |
| 491 | 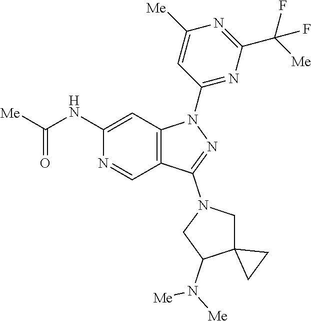 [SMILES: CC(=O)Nc1cc2c(cn1)c(N1CC(N(C)C)C3(CC3)C1)nn2-c1cc(C)nc(C(C)(F)F)n1] | LCMS m/z = 471.1 [M + H]⁺ 1H NMR (500 MHz, CDCl₃) δ ppm 9.34-9.68 (bs, 1H), 8.57- 8.92 (bs, 1H), 8.13-8.55 (m, 1H), 7.65 (s, 1H), 4.08 (d, J = 10 Hz, 2H), 3.78-3.93 (m, 1H), 3.38 (d, J = 5 Hz, 1H), 2.57-2.80 (m, 1H), 2.65 (s, 3H), 2.37-2.51 (m, 6H), 2.20-2.37 (m, 6H), 1.14-1.37 (m, 1H), 0.92-1.06 (m, 1H), 0.65- 0.79 (m, 2H). |
| | N-(1-(2-(1,1-difluoroethyl)-6- | |
| | methylpyrimidin-4-y1)-3-(7- | |
| | (dimethylamino)-5-azaspiro[2.4]heptan-5- | |
| | y1)-1H-pyrazolo[4,3-c]pyridin-6- | |
| | yl)acetamide | |
| 492 | 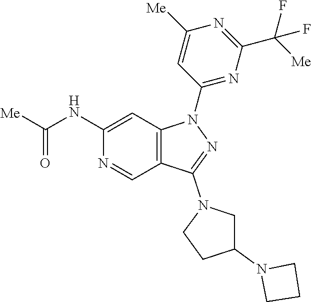 [SMILES: CC(=O)Nc1cc2c(cn1)c(N1CCC(N3CCC3)C1)nn2-c1cc(C)nc(C(C)(F)F)n1] | LCMS m/z = 457.1 [M + H]⁺ 1H NMR (500 MHz, CDCl₃) δ ppm 9.42 (s, 1H), 8.68 (s, 1H), 8.27 (s, 1H), 7.61 (s, 1H), 3.85- 3.97 (m, 1H), 3.73-3.85 (m, 1H), 3.62-3.73 (m, 1H), 3.51-3.60 (m, 1H), 3.40 (bs, 3H), 3.11-3.31 (m, 1H), 2.64 (s, 3H), 2.23-2.40 (m, 6H), 2.12-2.23 (m, 2H), 1.95- 2.12 (m, 2H). |
| | 7-N-(3-(3-(azetidin-1-yl)pyrrolidin-1-yl)-1- | |
| | (2-(1,1-difluoroethyl)-6-methylpyrimidin- | |
| | 4-y1)-1H-pyrazolo[4,3-c]pyridin-6- | |
| | yl)acetamide | |
| 493 | 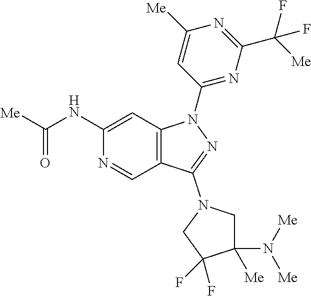 [SMILES: CC(=O)Nc1cc2c(cn1)c(N1CC(F)(F)C(C)(N(C)C)C1)nn2-c1cc(C)nc(C(C)(F)F)n1] | LCMS m/z = 495.1 [M + H]⁺ 1H NMR (500 MHz, CDCl₃) δ ppm 9.55 (s, 1H), 9.04 (s, 1H), 8.60 (s, 1H), 7.64 (s, 1H), 4.18- 4.22 (m, 1H), 4.10-4.15 (m, 1H), 3.91 (s, 2H), 2.66 (s, 3H), 2.42 (s, 6H), 2.29 (t, J = 20.0 Hz, 6H), 2.14 (s, 2H), 1.34 (s, 3H). |
| | N-(1-(2-(1,1-difluoroethyl)-6- | |
| | methylpyrimidin-4-y1)-3-(3- | |
| | (dimethylamino)-4,4-difluoro-3- | |
| | methylpyrrolidin-1-yl)-1H-pyrazolo[4,3- | |
| | c]pyridin-6-yl)acetamide | |
| 494 | 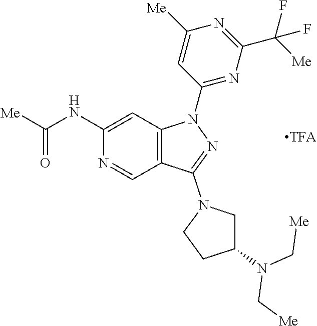 [SMILES: CCN(CC)[C@@H]1CCN(c2nn(-c3cc(C)nc(C(C)(F)F)n3)c3cc(NC(C)=O)ncc23)C1] | LCMS m/z = 473 [M + H]⁺ |
| | (R)-N-(3-(3-(diethylamino)pyrrolidin-1- | |
| | y1)-1-(2-(1,1-difluoroethyl)-6- | |
| | methylpyrimidin-4-yl)-1H-pyrazolo[4,3- | |
| | c]pyridin-6-yl)acetamide trifluoroacetate | |
| 495 | 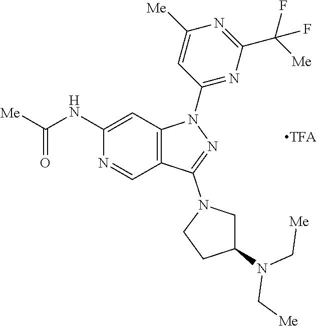 [SMILES: CCN(CC)[C@H]1CCN(c2nn(-c3cc(C)nc(C(C)(F)F)n3)c3cc(NC(C)=O)ncc23)C1] | LCMS m/z = 473 [M + H]⁺ |
| | (S)-N-(3-(3-(diethylamino)pyrrolidin-1- | |
| | yl)-1-(2-(1,1-difluoroethyl)-6- | |
| | methylpyrimidin-4-y1)-1H-pyrazolo[4,3- | |
| | c]pyridin-6-yl)acetamide trifluoroacetate | |
| 496 | 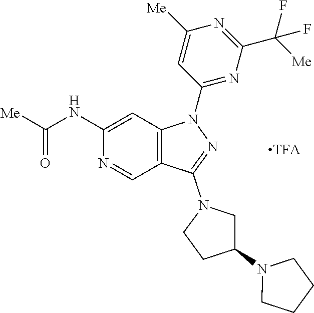 [SMILES: CC(=O)Nc1cc2c(cn1)c(N1CC[C@H](N3CCCC3)C1)nn2-c1cc(C)nc(C(C)(F)F)n1] | LCMS m/z = 471 [M + H]⁺ |
| | (S)-N-(3-([1,3′-bipyrrolidin]-1′-y1)-1-(2- | |
| | (1,1-difluoroethyl)-6-methylpyrimidin-4- | |
| | yl)-1H-pyrazolo[4,3-c]pyridin-6- | |
| | yl)acetamide trifluoroacetate | |
| 497 | 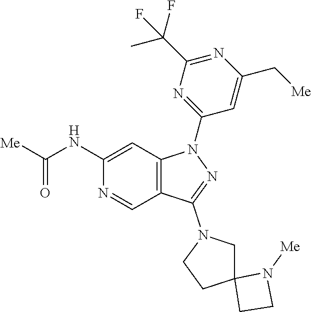 [SMILES: CCc1cc(-n2nc(N3CCC4(CCN4C)C3)c3cnc(NC(C)=O)cc32)nc(C(C)(F)F)n1] | LCMS m/z = 471.2 [M + H]⁺ 1H NMR (500 MHz, CDCl₃) δ ppm 9.48 (br s, 1H), 8.73 (s, 1H), 8.27 (br s, 1H), 7.63 (s, 1H), 3.95 (br d, J = 10.2 Hz, 1H), 3.85-3.92 (m, 1H), 3.65-3.81 (m, 2H), 3.34- 3.42 (m, 1H), 3.26-3.34 (m, 1H), 2.91 (q, J = 7.5 Hz, 2H), 2.40- 2.46 (m, 1H), 2.37 (br s, 3H), 2.22- 2.34 (m, 9 H), 2.17 (br dd, J = 11.3, 5.6 Hz, 1H), 1.39 (br t, J = 7.6 Hz, 4H). |
| | N-(1-(2-(1,1-difluoroethyl)-6- | |
| | ethylpyrimidin-4-yl)-3-(1-methyl-1,6- | |
| | diazaspiro[3.4]octan-6-y1)-1H- | |
| | pyrazolo[4,3-c]pyridin-6-yl)acetamide | |
| 498 | 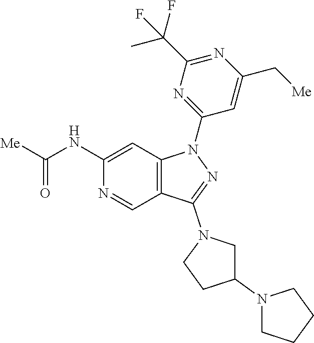 [SMILES: CCc1cc(-n2nc(N3CCC(N4CCCC4)C3)c3cnc(NC(C)=O)cc32)nc(C(C)(F)F)n1] | LCMS m/z = 485.2 [M + H]⁺ 1H NMR (500 MHz, CDCl₃) δ ppm 9.46 (br s, 1H), 8.65 (br s, 1H), 8.22 (br s, 1H), 7.58 (s, 1H), 3.99 (br s, 3H), 3.65-3.79 (m, 1H), 3.46-3.65 (m, 1H), 2.97- 3.39 (m, 3H), 2.92 (q, J = 7.6 Hz, 2H), 2.51-2.70 (m, 1H), 2.45 (br s, 1H), 2.25-2.33 (m, 7 H), 2.13 (br s, 4H), 1.41 (t, J = 7.6 Hz, 3H). |
| | N-(3-([1,3′-bipyrrolidin]-1′-y1)-1-(2-(1,1- | |
| | difluoroethyl)-6-ethylpyrimidin-4-yl)-1H- | |
| | pyrazolo[4,3-c]pyridin-6-yl)acetamide | |
| 499 | 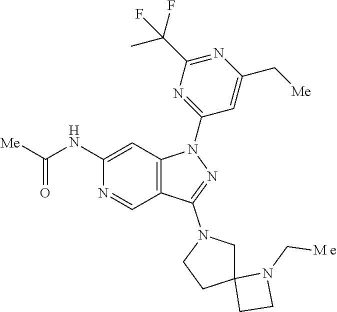 | LCMS m/z = 485.2 [M + H]⁺ 1H NMR (400 MHz, CDCl₃) δ ppm 9.47 (s, 1H), 8.74 (s, 1H), 8.53 (s, 1H), 7.50 (s, 1H), 4.21- 4.32 (m, 2H), 3.54 (br d, J = 11.0 Hz, 1H), 3.29-3.42 (m, 1H), 3.24 (br s, 2H), 2.75-2.96 (m, 6H), 2.62 (ddd, J = 11.3, 8.5, 5.3 Hz, 1H), 2.32-2.23 (m, 6H), 2.09 (br s, 1H), 1.38 (t, J = 7.5 Hz, 3H), 1.29 (br t, J = 7.3 Hz, 3H). |
| | N-(1-(2-(1,1-difluoroethyl)-6- | |
| | ethylpyrimidin-4-yl)-3-(1-ethyl-1,6- | |
| | diazaspiro[3.4]octan-6-y1)-1H- | |
| | pyrazolo[4,3-c]pyridin-6-yl)acetamide | |
| 500 | 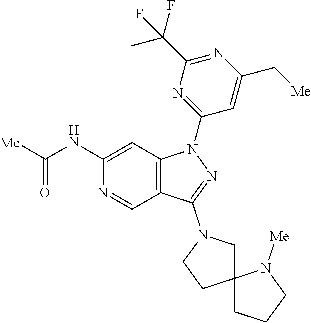 [SMILES: CCc1cc(-n2nc(N3CCC4(CCCN4C)C3)c3cnc(NC(C)=O)cc32)nc(C(C)(F)F)n1] | LCMS m/z = 485.3 [M + H]⁺ 1H NMR (400 MHz, CDCl₃) δ ppm 9.51 (s, 1H), 8.76 (s, 1H), 8.10 (s, 1H), 7.66 (s, 1H), 3.96 (td, J = 9.3, 3.5 Hz, 1H), 3.69-3.85 (m, 2H), 3.46-3.56 (m, 1H), 2.83- 3.00 (m, 4H), 2.46 (s, 3H), 2.24- 2.36 (m, 6H), 1.81-2.07 (m, 6H), 1.40 (t, J = 7.8 Hz, 3H). |
| | N-(1-(2-(1,1-difluoroethyl)-6- | |
| | ethylpyrimidin-4-y1)-3-(1-methyl-1,7- | |
| | diazaspiro[4.4]nonan-7-yl)-1H- | |
| | pyrazolo[4,3-c]pyridin-6-yl)acetamide | |
| 501 | 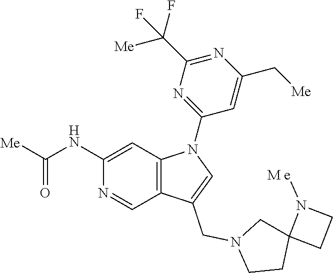 | LCMS m/z = 484.2 [M + H]⁺ 1H NMR (500 MHz, CDCl₃) 8 ppm 9.24 (s, 1H), 8.74 (s, 1H), 8.53 (br s, 1H), 7.72 (s, 1H), 7.35 (s, 1H), 3.70-3.84 (q, J = 7.5 Hz, 2H), 3.01-3.10 (m, 1H), 2.90-3.00 (m, 4H), 2.58-2.74 (m, 1H), 2.42- 2.52 (m, 2H), 2.21-2.27 (m, 9 H), 2.07-2.21 (m, 3H), 1.89 (ddd, J = 13.3, 7.8, 5.5 Hz, 1H), 1.42 (t, J = 7.5 Hz, 3H). |
| | N-(1-(2-(1,1-difluoroethyl)-6- | |
| | ethylpyrimidin-4-yl)-3-((1-methyl-1,6- | |
| | diazaspiro[3.4]octan-6-yl)methyl)-1H- | |
| | pyrrolo[3,2-c]pyridin-6-yl)acetamide | |
| 502 | 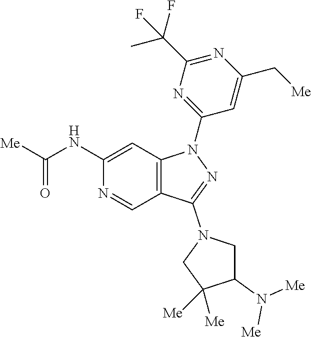 [SMILES: CCc1cc(-n2nc(N3CC(N(C)C)C(C)(C)C3)c3cnc(NC(C)=O)cc32)nc(C(C)(F)F)n1] | LCMS m/z = 487.2 [M + H]⁺ 1H NMR (500 MHz, CDCl₃) δ ppm 9.49 (br s, 1H), 8.72 (s, 1H), 8.09 (br s, 1H), 7.64 (s, 1H), 3.96 (br t, J = 8.4 Hz, 1H), 3.71-3.80 (m, 2H), 3.62 (d, J = 9.5 Hz, 1H), 3.55 (br d, J = 9.5 Hz, 1H), 2.91 (q, J = 7.6 Hz, 2H), 2.63 (s, 3H), 2.37 (s, 6H), 2.22-2.35 (m, 6H), 1.34- 1.44 (t, J = 7.6 Hz, 3H), 1.31 (s, 3H), 1.24 (s, 3H). |
| | N-(1-(2-(1,1-difluoroethyl)-6- | |
| | ethylpyrimidin-4-yl)-3-(4- | |
| | (dimethylamino)-3,3-dimethylpyrrolidin- | |
| | 1-y1)-1H-pyrazolo[4,3-c]pyridin-6- | |
| | yl)acetamide | |
| 503 | 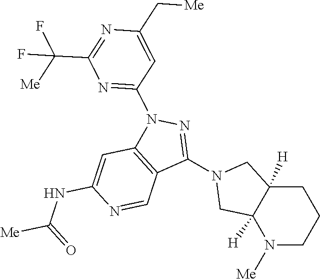 [SMILES: CCc1cc(-n2nc(N3C[C@H]4CCCN(C)[C@H]4C3)c3cnc(NC(C)=O)cc32)nc(C(C)(F)F)n1] | LCMS m/z = 485.2 [M + H]⁺ 1H NMR (500 MHz, CDCl₃) δ ppm 9.49 (br s, 1H), 8.74 (s, 1H), 8.21 (br s, 1H), 7.65 (s, 1H), 3.99 (br d, J = 10.7 Hz, 1H), 3.84-3.92 (m, 1H), 3.79 (br t, J = 8.2 Hz, 1H), 3.69 (dd, J = 10.7, 4.9 Hz, 1H), 2.82- 2.97 (m, 4H), 2.60 (br dd, J = 7.9, 3.7 Hz, 1H), 2.39 (s, 3H), 2.22- 2.36 (m, 6H), 1.71-1.93 (m, 4H), 1.66 (br dd, J = 9.3, 3.8 Hz, 1H), 1.40 (t, J = 7.6 Hz, 3H) |
| | N-(1-(2-(1,1-difluoroethyl)-6- | |
| | ethylpyrimidin-4-y1)-3-((4aR,7aR)-1- | |
| | methyloctahydro-6H-pyrrolo[3,4- | |
| | b]pyridin-6-yl)-1H-pyrazolo[4,3- | |
| | c]pyridin-6-yl)acetamide | |
| 504 | 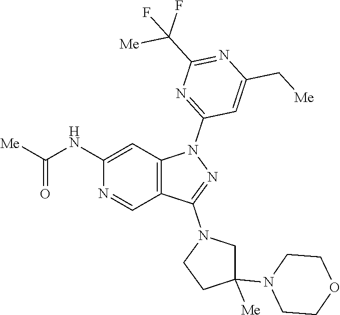 [SMILES: CCc1cc(-n2nc(N3CCC(C)(N4CCOCC4)C3)c3cnc(NC(C)=O)cc32)nc(C(C)(F)F)n1] | LCMS m/z = 515.2 [M + H]⁺ 1H NMR (500 MHz, CDCl₃) δ ppm 9.48 (br s, 1H), 8.70 (s, 1H), 8.28 (br s, 1H), 7.62 (s, 1H), 3.94 (t, J = 10.0 Hz, 1H), 3.73-3.87 (m, 5H), 3.61-3.72 (m, 2H), 2.90 (q, J = 7.4 Hz, 2H), 2.65-2.73 (m, 2H), 2.59-2.63 (m, 2H), 2.22-2.33 (m, 6H), 2.16 (q, J = 10.3 Hz, 1H), 2.03 (br dd, J = 10.7, 7.0 Hz, 1H), 1.38 (t, J = 7.6 Hz, 3H), 1.22 (s, 3H) |
| | N-(1-(2-(1,1-difluoroethyl)-6- | |
| | ethylpyrimidin-4-yl)-3-(3-methyl-3- | |
| | morpholinopyrrolidin-1-yl)-1H- | |
| | pyrazolo[4,3-c]pyridin-6-yl)acetamide | |
| 505 | 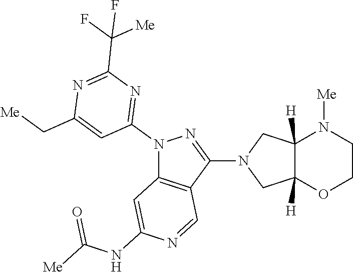 [SMILES: CCc1cc(-n2nc(N3C[C@H]4OCCN(C)[C@H]4C3)c3cnc(NC(C)=O)cc32)nc(C(C)(F)F)n1] | LCMS m/z = 487.2 [M + H]⁺ 1H NMR (500 MHz, CDCl₃) δ ppm 9.46 (br s, 1H), 8.70 (s, 1H), 8.38 (br s, 1H), 7.62 (s, 1H), 4.28 (br s, 1H), 3.83-3.98 (m, 4H), 3.67-3.79 (m, 2H), 3.46-3.58 (m, 1H), 2.90 (q, J = 7.6 Hz, 2H), 2.82 (br t, J = 10.1 Hz, 1H), 2.48- 2.63 (m, 4H), 2.21-2.33 (m, 6H), 1.38 (t, J = 7.6 Hz, 3H) |
| | N-(1-(2-(1,1-difluoroethyl)-6- | |
| | ethylpyrimidin-4-yl)-3-((4aS,7aR)-4- | |
| | methylhexahydropyrrolo[3,4- | |
| | b][1,4]oxazin-6(2H)-yl)-1H-pyrazolo[4,3- | |
| | c]pyridin-6-yl)acetamide | |
| 506 | 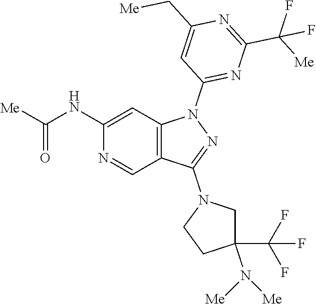 [SMILES: CCc1cc(-n2nc(N3CCC(N(C)C)(C(F)(F)F)C3)c3cnc(NC(C)=O)cc32)nc(C(C)(F)F)n1] | LCMS m/z = 527.1 [M + H]⁺ 1H NMR (500 MHz, CDCl₃) δ ppm 9.49 (br s, 1H), 8.69 (s, 1H), 8.35 (br s, 1H), 7.64 (s, 1H), 4.10 (br d, J = 11.3 Hz, 1H), 3.96-4.02 (m, 1H), 3.84-3.96 (m, 2H), 2.91 (q, J = 7.6 Hz, 2H), 2.57 (br s, 6H), 2.53-2.56 (m, 1H), 2.42 (dt, J = 13.6, 8.2 Hz, 1H), 2.23-2.33 (m, 6H), 1.39 (t, J = 7.6 Hz, 3H). |
| | N-(1-(2-(1,1-difluoroethyl)-6- | |
| | ethylpyrimidin-4-yl)-3-(3- | |
| | (dimethylamino)-3- | |
| | (trifluoromethyl)pyrrolidin-1-y1)-1H- | |
| | pyrazolo[4,3-c]pyridin-6-yl)acetamide | |
| 507 | 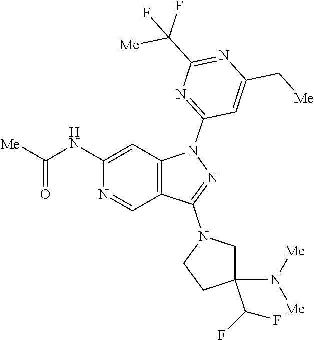 [SMILES: CCc1cc(-n2nc(N3CCC(C(F)F)(N(C)C)C3)c3cnc(NC(C)=O)cc32)nc(C(C)(F)F)n1] | LCMS m/z = 509.3 [M + H]⁺ 1H NMR (600 MHz, CDCl₃) δ ppm 9.54 (br s, 1H), 9.32 (br s, 1H), 8.63 (s, 1H), 7.64 (s, 1H), 6.08 (t, J = 54.0 Hz, 1H), 4.03 (d, J = 10.5 Hz, 1H), 3.97 (td, J = 9.0, 3.5 Hz, 1H), 3.87 (q, J = 8.4 Hz, 1H), 3.79 (br d, J = 10.5 Hz, 1H), 2.92 (q, J = 7.6 Hz, 2H), 2.55 (s, 6H), 2.42- 2.52 (m, 1H), 2.24-2.34 (m, 7 H), 1.39 (t, J = 7.6 Hz, 3H). |
| | N-(1-(2-(1,1-difluoroethyl)-6- | |
| | ethylpyrimidin-4-y1)-3-(3- | |
| | (difluoromethyl)-3- | |
| | (dimethylamino)pyrrolidin-1-yl)-1H- | |
| | pyrazolo[4,3-c]pyridin-6-yl)acetamide | |
| 508 | 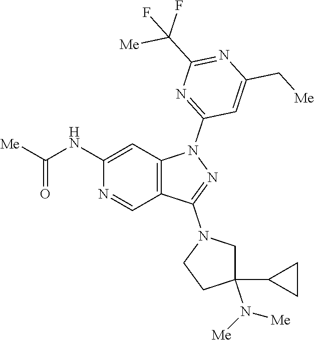 [SMILES: CCc1cc(-n2nc(N3CCC(C4CC4)(N(C)C)C3)c3cnc(NC(C)=O)cc32)nc(C(C)(F)F)n1] | LCMS m/z = 499.3 [M + H]⁺ 1H NMR (500 MHz, CDCl₃) δ ppm 9.47 (br s, 1H), 8.70 (s, 1H), 8.42 (br s, 1H), 7.62 (s, 1H), 3.90 (td, J = 8.9, 4.3 Hz, 1H), 3.79 (q, J = 8.1 Hz, 1H), 3.64 (d, J = 10.4 Hz, 1H), 3.34 (d, J = 10.4 Hz, 1H), 2.90 (q, J = 7.6 Hz, 2H), 2.47 (s, 6H), 2.21-2.35 (m, 7 H), 2.04 (ddd, J = 12.4, 7.8, 4.3 Hz, 1H), 1.38 (t, J = 7.6 Hz, 3H), 1.10 (tt, J = 8.4, 5.6 Hz, 1H), 0.54-0.61 (m, 1H), 0.46-0.54 (m, 1H), 0.42 (dq, J = 9.8, 5.2 Hz, 1H), 0.14-0.20 (m, 1H). |
| | N-(3-(3-cyclopropyl-3- | |
| | (dimethylamino)pyrrolidin-1-yl)-1-(2- | |
| | (1,1-difluoroethyl)-6-ethylpyrimidin-4- | |
| | yl)-1H-pyrazolo[4,3-c]pyridin-6- | |
| | yl)acetamide | |
| 509 | 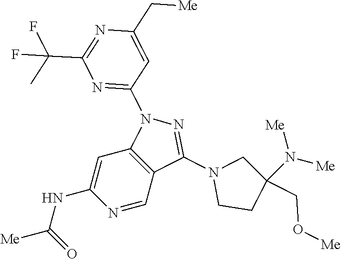 [SMILES: CCc1cc(-n2nc(N3CCC(COC)(N(C)C)C3)c3cnc(NC(C)=O)cc32)nc(C(C)(F)F)n1] | LCMS m/z = 503.1 [M + H]⁺ 1H NMR (500 MHz, CDCl₃) δ ppm 9.47 (br s, 1H), 8.73 (br s, 1H), 8.68 (br s, 1H), 7.63 (s, 1H), 3.85-3.91 (m, 1H), 3.75-3.85 (m, 2H), 3.69 (br d, J = 10.1 Hz, 1H), 3.50-3.58 (m, 2H), 3.36 (s, 3H), 2.90 (q, J = 7.5 Hz, 2H), 2.44 (s, 6H), 2.17-2.34 (m, 8H), 1.34- 1.42 (t, J = 7.5 Hz, 3H). |
| | N-(1-(2-(1,1-difluoroethyl)-6- | |
| | ethylpyrimidin-4-yl)-3-(3- | |
| | (dimethylamino)-3- | |
| | (methoxymethyl)pyrrolidin-1-yl)-1H- | |
| | pyrazolo[4,3-c]pyridin-6-yl)acetamide | |
| 510 | 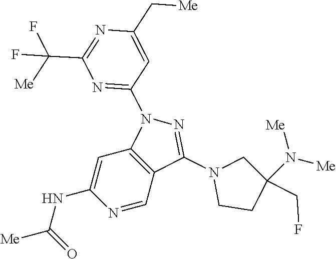 [SMILES: CCc1cc(-n2nc(N3CCC(CF)(N(C)C)C3)c3cnc(NC(C)=O)cc32)nc(C(C)(F)F)n1] | LCMS m/z = 491.2 [M + H]⁺ 1H NMR (400 MHz, CDCl₃) δ ppm 9.48 (br s, 1H), 8.71 (s, 1H), 8.36 (br s, 1H), 7.62 (s, 1H), 4.48- 4.77 (m, 2H), 3.96 (td, J = 9.5, 3.0 Hz, 1H), 3.79-3.91 (m, 2H), 3.66 (dd, J = 10.5, 5.0 Hz, 1H), 2.91 (q, J = 7.7 Hz, 2H), 2.45-2.52 (m, 6H), 2.18-2.36 (m, 8H), 1.38 (t, J = 7.8 Hz, 3H). |
| | N-(1-(2-(1,1-difluoroethyl)-6- | |
| | ethylpyrimidin-4-y1)-3-(3- | |
| | (dimethylamino)-3- | |
| | (fluoromethyl)pyrrolidin-1-y1)-1H- | |
| | pyrazolo[4,3-c]pyridin-6-yl)acetamide | |
| 511 | 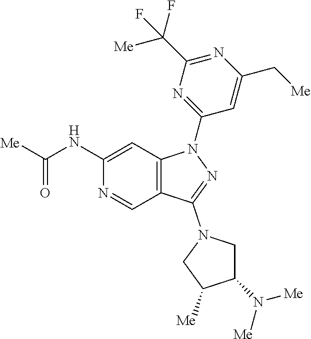 [SMILES: CCc1cc(-n2nc(N3C[C@@H](C)[C@@H](N(C)C)C3)c3cnc(NC(C)=O)cc32)nc(C(C)(F)F)n1] | LCMS m/z = 473.3 [M + H]⁺ 1H NMR (400 MHz, CDCl₃) δ ppm 9.47 (br s, 1H), 8.70 (br s, 1H), 8.31 (br s, 1H), 7.62 (s, 1H), 3.79-3.96 (m, 2H), 3.61-3.76 (m, 2H), 2.91 (q, J = 7.5 Hz, 3H), 2.50-2.66 (m, 2H), 2.44 (s, 6H), 2.21-2.34 (m, 6H), 1.39 (t, J = 7.5 Hz, 3H), 1.17 (d, J = 7.0 Hz, 3H) |
| | or | |
| | 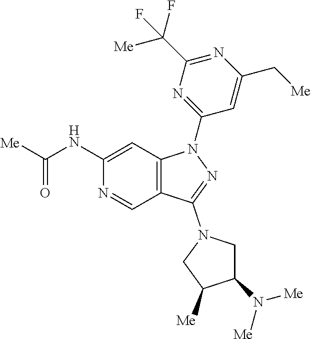 [SMILES: CCc1cc(-n2nc(N3C[C@@H](N(C)C)[C@@H](C)C3)c3cnc(NC(C)=O)cc32)nc(C(C)(F)F)n1] | |
| | N-(1-(2-(1,1-difluoroethyl)-6- | |
| | ethylpyrimidin-4-yl)-3-((3R,4R)-3- | |
| | (dimethylamino)-4-methylpyrrolidin-1- | |
| | y1)-1H-pyrazolo[4,3-c]pyridin-6- | |
| | yl)acetamide or N-(1-(2-(1,1- | |
| | difluoroethyl)-6-ethylpyrimidin-4-yl)-3- | |
| | ((3S,4S)-3-(dimethylamino)-4- | |
| | methylpyrrolidin-1-yl)-1H-pyrazolo[4,3- | |
| | c]pyridin-6-yl)acetamide | |
| 512 | 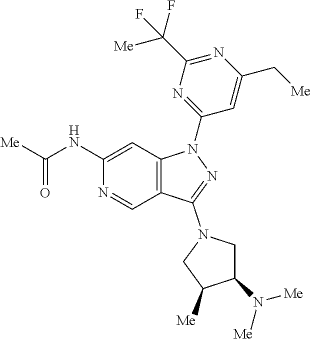 [SMILES: CCc1cc(-n2nc(N3C[C@@H](N(C)C)[C@@H](C)C3)c3cnc(NC(C)=O)cc32)nc(C(C)(F)F)n1] | LCMS m/z = 473.3 [M + H]⁺ 1H NMR (400 MHz, CDCl₃) δ ppm 9.47 (br s, 1H), 8.70 (br s, 1H), 8.31 (br s, 1H), 7.62 (s, 1H), 3.79-3.96 (m, 2H), 3.61-3.76 (m, 2H), 2.91 (q, J = 7.5 Hz, 3H), 2.50-2.66 (m, 2H), 2.44 (s, 6H), 2.21-2.34 (m, 6H), 1.39 (t, J = 7.5 Hz, 3H), 1.17 (d, J = 7.0 Hz, 3H) |
| | or | |
| | 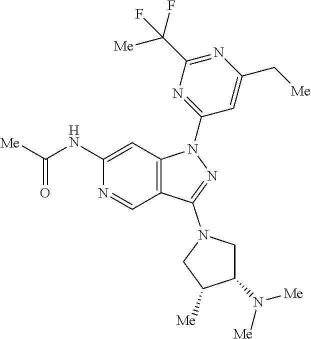 [SMILES: CCc1cc(-n2nc(N3C[C@@H](C)[C@@H](N(C)C)C3)c3cnc(NC(C)=O)cc32)nc(C(C)(F)F)n1] | |
| | N-(1-(2-(1,1-difluoroethyl)-6- | |
| | ethylpyrimidin-4-yl)-3-((3S,4S)-3- | |
| | (dimethylamino)-4-methylpyrrolidin-1- | |
| | yl)-1H-pyrazolo[4,3-c]pyridin-6- | |
| | yl)acetamide or N-(1-(2-(1,1- | |
| | difluoroethyl)-6-ethylpyrimidin-4-yl)-3- | |
| | ((3R,4R)-3-(dimethylamino)-4- | |
| | methylpyrrolidin-1-yl)-1H-pyrazolo[4,3- | |
| | c]pyridin-6-yl)acetamide | |
| 513 | 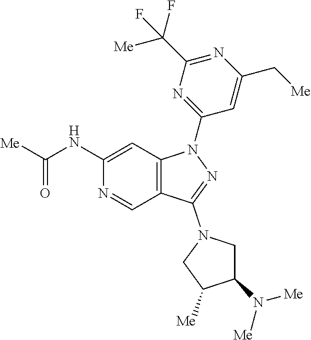 [SMILES: CCc1cc(-n2nc(N3C[C@@H](C)[C@H](N(C)C)C3)c3cnc(NC(C)=O)cc32)nc(C(C)(F)F)n1] | LCMS m/z = 473.3 [M + H]⁺ 1H NMR (400 MHz, CDCl₃) δ ppm 9.47 (br s, 1H), 8.72 (s, 1H), 8.32 (br s, 1H), 7.63 (s, 1H), 4.01 (dd, J = 9.5, 8.0 Hz, 1H), 3.85 (dd, J = 10.3, 7.3 Hz, 1 H), 3.73 (dd, J = 10.0, 6.5 Hz, 1H), 3.35 (dd, J = 9.5, 7.5 Hz, 1H), 2.86-3.00 (m, 3H), 2.53 (dt, J = 14.1, 7.2 Hz, 1H), 2.43 (s, 6H), 2.17-2.38 (m, 6H), 1.38 (t, J = 7.5 Hz, 3H), 1.24 (d, J = 7.0 Hz, 3H) |
| | or | |
| | 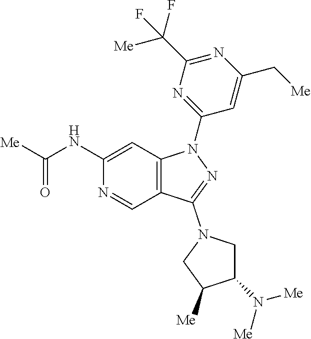 [SMILES: CCc1cc(-n2nc(N3C[C@H](C)[C@@H](N(C)C)C3)c3cnc(NC(C)=O)cc32)nc(C(C)(F)F)n1] | |
| | N-(1-(2-(1,1-difluoroethyl)-6- | |
| | ethylpyrimidin-4-yl)-3-((3S,4R)-3- | |
| | (dimethylamino)-4-methylpyrrolidin-1- | |
| | yl)-1H-pyrazolo[4,3-c]pyridin-6- | |
| | yl)acetamide or N-(1-(2-(1,1- | |
| | difluoroethyl)-6-ethylpyrimidin-4-yl)-3- | |
| | ((3R,4S)-3-(dimethylamino)-4- | |
| | methylpyrrolidin-1-yl)-1H-pyrazolo[4,3- | |
| | c]pyridin-6-yl)acetamide | |
| 514 | 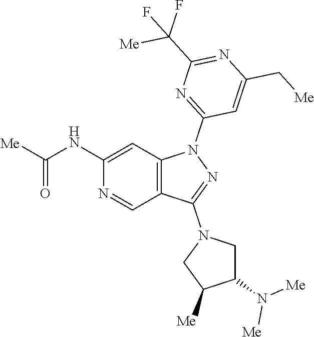 | LCMS m/z = 485.2 [M + H]⁺ 1H NMR (400 MHz, CDCl₃) δ ppm 9.47 (br s, 1H), 8.72 (s, 1H), 8.32 (br s, 1H), 7.63 (s, 1H), 4.01 (dd, J = 9.5, 8.0 Hz, 1H), 3.85 (dd, J = 10.3, 7.3 Hz, 1H), 3.73 (dd, J = 10.0, 6.5 Hz, 1H), 3.35 (dd, J = 9.5, 7.5 Hz, 1H), 2.86-3.00 (m, 3H), 2.53 (dt, J = 14.1, 7.2 Hz, 1H), 2.43 (s, 6H), 2.17-2.38 (m, 6H), 1.38 (t, J = 7.5 Hz, 3H), 1.24 (d, J = 7.0 Hz, 3H) |
| | or | |
| | 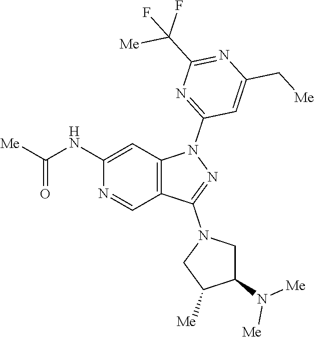 [SMILES: CCc1cc(-n2nc(N3C[C@@H](C)[C@H](N(C)C)C3)c3cnc(NC(C)=O)cc32)nc(C(C)(F)F)n1] | |
| | N-(1-(2-(1,1-difluoroethyl)-6- | |
| | ethylpyrimidin-4-yl)-3-((3R,4S)-3- | |
| | (dimethylamino)-4-methylpyrrolidin-1- | |
| | yl)-1H-pyrazolo[4,3-c]pyridin-6- | |
| | yl)acetamide or N-(1-(2-(1,1- | |
| | difluoroethyl)-6-ethylpyrimidin-4-yl)-3- | |
| | ((3S,4R)-3-(dimethylamino)-4- | |
| | methylpyrrolidin-1-y1)-1H-pyrazolo[4,3- | |
| | c]pyridin-6-yl)acetamide | |
| 515 | 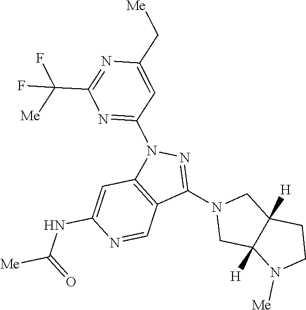 [SMILES: CCc1cc(-n2nc(N3C[C@@H]4CCN(C)[C@@H]4C3)c3cnc(NC(C)=O)cc32)nc(C(C)(F)F)n1] | LCMS m/z = 471.2 [M + H]⁺ 1H NMR (500 MHz, CDCl₃) δ ppm 9.45 (br s, 1H), 8.72 (s, 1H), 8.49 (br s, 1H), 7.63 (s, 1H), 3.82- 3.91 (m, 2H), 3.67 (br dd, J = 9.6, 4.4 Hz, 1H), 3.60 (br dd, J = 10.1, 3.4 Hz, 1H), 3.20 (br t, J = 8.2 Hz, 1H), 3.01 (br s, 2H), 2.90 (q, J = 7.5 Hz, 2H), 2.44 (s, 3H), 2.37- 2.42 (m, 1H), 2.17-2.33 (m, 6H), 2.00-2.14 (m, 1H), 1.80-1.90 (m, 1H), 1.37 (t, J = 7.6 Hz, 3H). |
| | N-(1-(2-(1,1-difluoroethyl)-6- | |
| | ethylpyrimidin-4-y1)-3-((3aS,6aS)-1- | |
| | methylhexahydropyrrolo[3,4-b]pyrrol- | |
| | 5(1H)-y1)-1H-pyrazolo[4,3-c]pyridin-6- | |
| | yl)acetamide | |
| 516 | 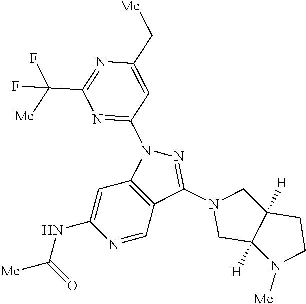 [SMILES: CCc1cc(-n2nc(N3C[C@H]4CCN(C)[C@H]4C3)c3cnc(NC(C)=O)cc32)nc(C(C)(F)F)n1] | LCMS m/z = 471.2 [M + H]⁺ 1H NMR (600 MHz, CDCl₃) δ ppm 9.48 (br s, 1H), 8.73 (s, 1H), 8.33 (br s, 1H), 7.64 (s, 1H), 3.82- 3.94 (m, 2H), 3.69 (dd, J = 9.6, 4.9 Hz, 1H), 3.62 (dd, J = 10.5, 4.7 Hz, 1H), 3.21 (br t, J = 8.0 Hz, 1H), 2.98- 3.06 (m, 2H), 2.91 (q, J = 7.6 Hz, 2H), 2.45 (s, 3H), 2.39-2.44 (m, 1H), 2.19-2.33 (m, 7 H), 1.82- 1.89 (m, 1H), 1.38 (t, J = 7.4 Hz, 3H). |
| | N-(1-(2-(1,1-difluoroethyl)-6- | |
| | ethylpyrimidin-4-yl)-3-((3aR,6aR)-1- | |
| | methylhexahydropyrrolo[3,4-b]pyrrol- | |
| | 5(1H)-yl)-1H-pyrazolo[4,3-c]pyridin-6- | |
| | yl)acetamide | |
| 517 | 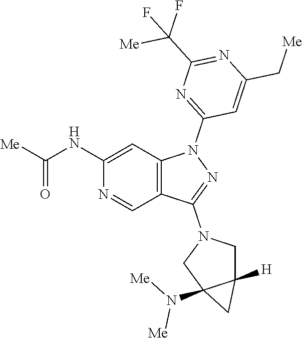 [SMILES: CCc1cc(-n2nc(N3C[C@@H]4C[C@]4(N(C)C)C3)c3cnc(NC(C)=O)cc32)nc(C(C)(F)F)n1] | LCMS m/z = 471.2 [M + H]⁺ 1H NMR (600 MHz, CDCl₃) δ ppm 9.50 (br s, 1H), 8.72 (br s, 1H), 8.13 (br s, 1H), 7.63 (s, 1H), 3.81-3.92 (m, 4H), 2.92 (q, J = 7.6 Hz, 2H), 2.54 (br s, 6H), 2.24-2.32 (m, 6H), 1.76-1.85 (m, 1H), 1.39 (t, J = 7.6 Hz, 3H), 1.10- 1.26 (m, 1H), 0.76 (br s, 1H). |
| | N-(1-(2-(1,1-difluoroethyl)-6- | |
| | ethylpyrimidin-4-yl)-3-((1R,5S)-1- | |
| | (dimethylamino)-3- | |
| | azabicyclo[3.1.0]hexan-3-yl)-1H- | |
| | pyrazolo[4,3-c]pyridin-6-yl)acetamide | |
| 518 | 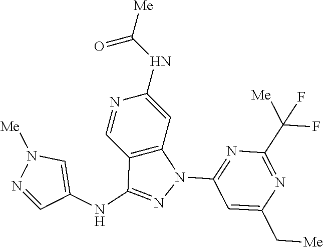 [SMILES: CCc1cc(-n2nc(Nc3cnn(C)c3)c3cnc(NC(C)=O)cc32)nc(C(C)(F)F)n1] | LCMS m/z = 441.2 [M + H]⁺ |
| | N-(1-(2-(1,1-difluoroethyl)-6- | |
| | ethylpyrimidin-4-y1)-3-((1-methyl-1H- | |
| | pyrazol-4-yl)amino)-1H-pyrazolo[4,3- | |
| | c]pyridin-6-yl)acetamide | |
| 519 | 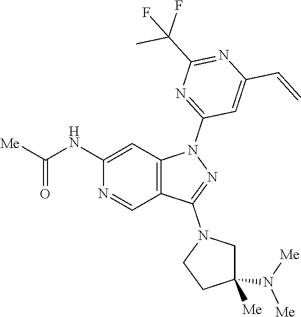 [SMILES: C=Cc1cc(-n2nc(N3CC[C@@](C)(N(C)C)C3)c3cnc(NC(C)=O)cc32)nc(C(C)(F)F)n1] | LCMS m/z = 471.0 [M + H]⁺ |
| | or | |
| | 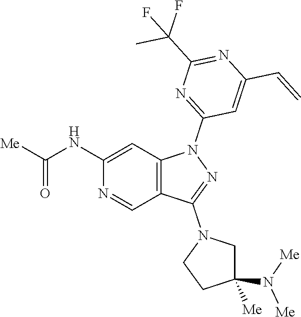 [SMILES: C=Cc1cc(-n2nc(N3CC[C@](C)(N(C)C)C3)c3cnc(NC(C)=O)cc32)nc(C(C)(F)F)n1] | |
| | (R)-N-(1-(2-(1,1-difluoroethyl)-6- | |
| | vinylpyrimidin-4-y1)-3-(3- | |
| | (dimethylamino)-3-methylpyrrolidin-1- | |
| | yl)-1H-pyrazolo[4,3-c]pyridin-6- | |
| | yl)acetamide or (S)-N-(1-(2-(1,1- | |
| | difluoroethyl)-6-vinylpyrimidin-4-yl)-3- | |
| | (3-(dimethylamino)-3-methylpyrrolidin-1- | |
| | y1)-1H-pyrazolo[4,3-c]pyridin-6- | |
| | yl)acetamide | |
| 520 | 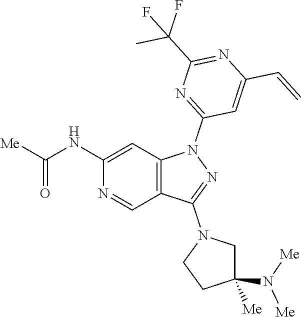 [SMILES: C=Cc1cc(-n2nc(N3CC[C@](C)(N(C)C)C3)c3cnc(NC(C)=O)cc32)nc(C(C)(F)F)n1] | LCMS m/z = 471.0 [M + H]+ |
| | or | |
| | 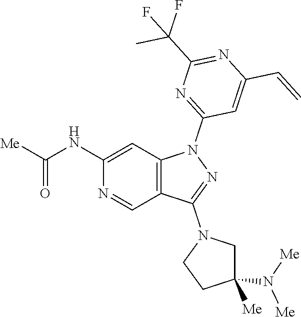 [SMILES: C=Cc1cc(-n2nc(N3CC[C@@](C)(N(C)C)C3)c3cnc(NC(C)=O)cc32)nc(C(C)(F)F)n1] | |
| | (S)-N-(1-(2-(1,1-difluoroethyl)-6- | |
| | vinylpyrimidin-4-yl)-3-(3- | |
| | (dimethylamino)-3-methylpyrrolidin-1- | |
| | y1)-1H-pyrazolo[4,3-c]pyridin-6- | |
| | yl)acetamide or (R)-N-(1-(2-(1,1- | |
| | difluoroethyl)-6-vinylpyrimidin-4-yl)-3- | |
| | (3-(dimethylamino)-3-methylpyrrolidin-1- | |
| | yl)-1H-pyrazolo[4,3-c]pyridin-6- | |
| | yl)acetamide | |
| 521 | 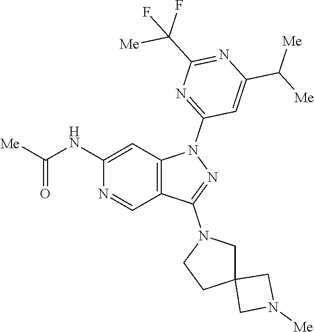 [SMILES: CC(=O)Nc1cc2c(cn1)c(N1CCC3(CN(C)C3)C1)nn2-c1cc(C(C)C)nc(C(C)(F)F)n1] | 1H NMR (400 MHz, CDCl₃) δ ppm 9.46 (br s, 1H), 8.68 (s, 1H), 8.47 (br s, 1H), 7.60 (s, 1H), 3.88 (s, 2H), 3.74 (t, J = 6.8 Hz, 2H), 3.33-3.48 (m, 4H), 3.12 (dt, J = 13.6, 6.9 Hz, 1H), 2.45 (s, 3H), 2.19-2.34 (m, 9 H), 1.37 (d, J = 7.0 Hz, 6H). |
| | N-(1-(2-(1,1-difluoroethyl)-6- | |
| | isopropylpyrimidin-4-yl)-3-(2-methyl-2,6- | |
| | diazaspiro[3.4]octan-6-y1)-1H- | |
| | pyrazolo[4,3-c]pyridin-6-yl)acetamide | |
| 522 | 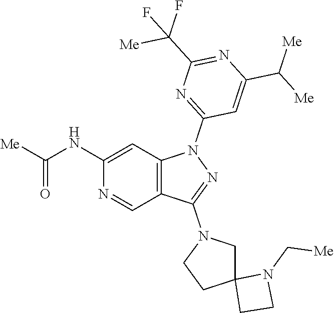 [SMILES: CCN1CCC12CCN(c1nn(-c3cc(C(C)C)nc(C(C)(F)F)n3)c3cc(NC(C)=O)ncc13)C2] | LCMS m/z = 498.9 [M + H]⁺ 1H NMR (500 MHz, CDCl₃) δ ppm 9.49 (br s, 1H), 8.73 (s, 1H), 8.17 (br s, 1H), 7.62 (s, 1H), 3.86- 4.01 (m, 2H), 3.71-3.82 (m, 2H), 3.23-3.51 (m, 2H), 3.13 (dt, J = 13.8, 7.0 Hz, 1H), 2.56-2.77 (m, 2H), 2.41-2.56 (m, 1H), 2.34- 2.40 (m, 1H), 2.18-2.34 (m, 8H), 1.38 (d, J = 7.0 Hz, 6H), 1.07 (t, J = 7.0 Hz, 3H). |
| | N-(1-(2-(1,1-difluoroethyl)-6- | |
| | isopropylpyrimidin-4-yl)-3-(1-ethyl-1,6- | |
| | diazaspiro[3.4]octan-6-yl)-1H- | |
| | pyrazolo[4,3-c]pyridin-6-yl)acetamide | |
| 523 | 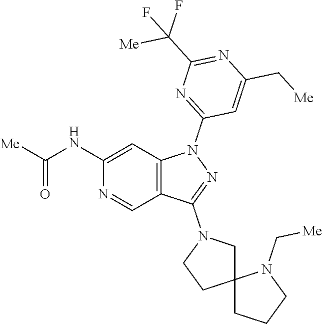 [SMILES: CCc1cc(-n2nc(N3CCC4(CCCN4CC)C3)c3cnc(NC(C)=O)cc32)nc(C(C)(F)F)n1] | LCMS m/z = 517.2 [M + H]⁺ 1H NMR (500 MHz, CDCl₃) δ ppm 9.53 (br s, 1H), 8.99 (br s, 1H), 8.68 (s, 1H), 7.62 (s, 1H), 3.92 (td, J = 9.2, 2.7 Hz, 1H), 3.69- 3.84 (m, 2H), 3.53 (d, J = 9.8 Hz, 1H), 3.07-3.19 (m, 1H), 2.95- 3.06 (m, 1H), 2.76-2.87 (m, 1H), 2.66-2.74 (m, 1H), 2.59-2.66 (m, 1H), 2.20-2.34 (m, 7 H), 1.99- 2.05 (m, 1H), 1.89-1.98 (m, 3H), 1.80-1.88 (m, 1H), 1.37 (d, J = 7.0 Hz, 6H), 1.18 (t, J = 7.2 Hz, 3H). |
| | N-(1-(2-(1,1-difluoroethyl)-6- | |
| | isopropylpyrimidin-4-yl)-3-(1-ethyl-1,7- | |
| | diazaspiro[4.4]nonan-7-y1)-1H- | |
| | pyrazolo[4,3-c]pyridin-6-yl)acetamide | |
| 524 | 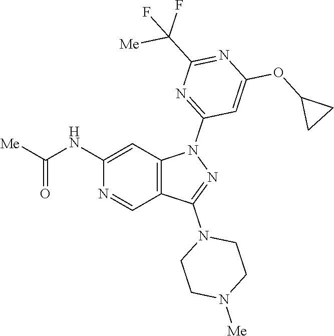 [SMILES: CC(=O)Nc1cc2c(cn1)c(N1CCN(C)CC1)nn2-c1cc(OC2CC2)nc(C(C)(F)F)n1] | LCMS m/z = 473.1 [M + H]⁺ 1H NMR (500 MHz, MeOD- d₄) ppm 9.35 (br s, 1H), 8.85 (s, 1H), 8.72 (d, J = 5.8 Hz, 1H), 7.81 (br d, J = 5.0 Hz, 1H), 3.85 (s, 2H), 3.76 (br t, J = 6.5 Hz, 2H), 3.42 (s, 4H), 2.44 (s, 3H), 2.26-2.32 (m, 3H), 2.14-2.26 (m, 5H). |
| | N-(1-(6-cyclopropoxy-2-(1,1- | |
| | difluoroethyl)pyrimidin-4-y1)-3-(4- | |
| | methylpiperazin-1-yl)-1H-pyrazolo[4,3- | |
| | c]pyridin-6-yl)acetamide | |
| 525 | 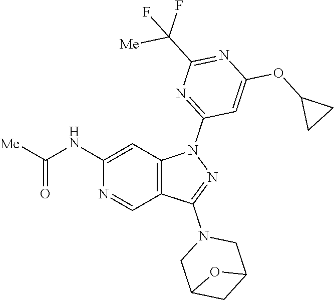 [SMILES: CC(=O)Nc1cc2c(cn1)c(N1CC3CC(C1)O3)nn2-c1cc(OC2CC2)nc(C(C)(F)F)n1] | LCMS m/z = 472.2 [M + H]⁺ 1H NMR (500 MHz, CDCl₃) δ ppm 10.1 (bs, 1H), 9.68 (s, 1H), 8.75 (s, 1H), 7.26 (s, 1H), 4.77- 4.91 (m, 2H), 4.37-4.48 (m, 1H), 4.01-4.11 (m, 2H), 3.88-4.01 (m, 2H), 3.33-3.47 (m, 1H), 2.30- 2.35 (m, 3H), 2.20-2.30 (m, 4H), 2.10-2.17 (m, 4H), 0.83- 0.93 (m, 4H). |
| | N-(3-(6-oxa-3-azabicyclo[3.1.1]heptan-3- | |
| | yl)-1-(6-cyclopropoxy-2-(1,1- | |
| | difluoroethyl)pyrimidin-4-y1)-1H- | |
| | pyrazolo[4,3-c]pyridin-6-yl)acetamide | |
| 526 | 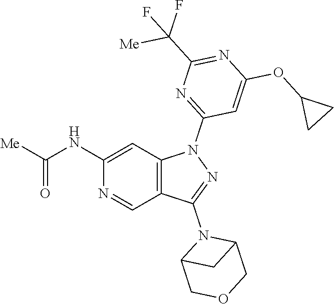 [SMILES: CC(=O)Nc1cc2c(cn1)c(N1C3COCC1C3)nn2-c1cc(OC2CC2)nc(C(C)(F)F)n1] | LCMS m/z = 472.0 [M + H]⁺ 1H NMR (500 MHz, CDCl₃) δ ppm 10.54-11.18 (b s, 1H), 9.87 (br s, 1H), 8.58 (br s, 1H), 7.23 (s, 1H), 4.58 (br d, J = 5.8 Hz, 2H), 4.47-4.52 (m, 1H), 4.43 (br d, J = 11.1 Hz, 2H), 3.94 (br d, J = 11.1 Hz, 2H), 3.10 (br d, J = 6.7 Hz, 1H), 2.39 (s, 3H), 2.24 (br t, J = 18.7 Hz, 3H), 2.14 (br d, J = 8.2 Hz, 1H), 0.82-0.96 (m, 4H). |
| | N-(3-(3-oxa-6-azabicyclo[3.1.1]heptan-6- | |
| | y1)-1-(6-cyclopropoxy-2-(1,1- | |
| | difluoroethyl)pyrimidin-4-y1)-1H- | |
| | pyrazolo[4,3-c]pyridin-6-yl)acetamide | |
| 527 | 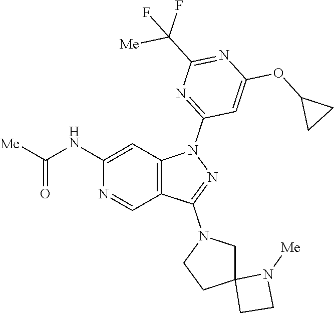 [SMILES: CC(=O)Nc1cc2c(cn1)c(N1CCC3(CCN3C)C1)nn2-c1cc(OC2CC2)nc(C(C)(F)F)n1] | LCMS m/z = 499.1 [M + H]⁺ 1H NMR (500 MHz, CDCl₃) δ ppm 9.47 (bs, 1H), 8.72 (s, 1H), 8.16 (bs, 1H), 7.21 (s, 1H), 4.39 (bs, 1H), 3.99-4.18 (m, 1H), 3.85- 3.99 (m, 1H), 3.68-3.82 (m, 2H), 3.32-3.56 (m, 2H), 2.38- 2.53 (m, 4H), 2.15-2.31 (m, 8H), 0.88 (bs, 4H). |
| | N-(1-(6-cyclopropoxy-2-(1,1- | |
| | difluoroethyl)pyrimidin-4-yl)-3-(1- | |
| | methyl-1,6-diazaspiro[3.4]octan-6-y1)-1H- | |
| | pyrazolo[4,3-c]pyridin-6-yl)acetamide | |
| 528 | 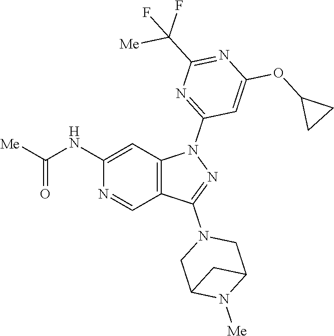 [SMILES: CC(=O)Nc1cc2c(cn1)c(N1CC3CC(C1)N3C)nn2-c1cc(OC2CC2)nc(C(C)(F)F)n1] | LCMS m/z = 485.2 [M + H]⁺ 1H NMR (500 MHz, CDCl₃) δ ppm 9.54 (br s, 1H), 8.86 (s, 1H), 8.23 (br s, 1H), 7.26 (s, 1H), 4.40 (br s, 1H), 4.08 (br d, J = 11.1 Hz, 2H), 3.78-3.90 (m, 4H), 2.83 (br s, 1H), 2.35 (br s, 3H), 2.23-2.33 (m, 6H), 1.81 (br d, J = 8.7 Hz, 1H), 0.89 (br s, 4H). |
| | N-(1-(6-cyclopropoxy-2-(1,1- | |
| | difluoroethyl)pyrimidin-4-yl)-3-(6- | |
| | methyl-3,6-diazabicyclo[3.1.1]heptan-3- | |
| | y1)-1H-pyrazolo[4,3-c]pyridin-6- | |
| | yl)acetamide | |
| 529 | 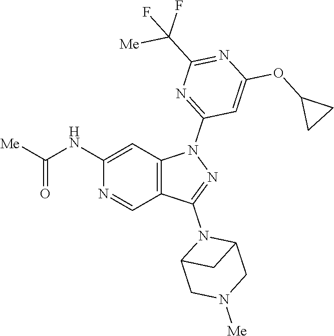 [SMILES: CC(=O)Nc1cc2c(cn1)c(N1C3CC1CN(C)C3)nn2-c1cc(OC2CC2)nc(C(C)(F)F)n1] | LCMS m/z = 485.2 [M + H]⁺ 1H NMR (500 MHz, CDCl₃) δ ppm 9.49 (br s, 1H), 8.49 (s, 1H), 8.14 (br s, 1H), 7.19 (s, 1H), 4.56 (br d, J = 4.7 Hz, 2H), 4.38-4.49 (m, 1H), 3.31 (br s, 2H), 3.14- 3.25 (m, 1H), 2.89 (br s, 1H), 2.41 (br s, 1H), 2.22-2.36 (m, 9 H), 0.89 (br s, 4H). |
| | N-(1-(6-cyclopropoxy-2-(1,1- | |
| | difluoroethyl)pyrimidin-4-y1)-3-(3- | |
| | methyl-3,6-diazabicyclo[3.1.1]heptan-6- | |
| | yl)-1H-pyrazolo[4,3-c]pyridin-6- | |
| | yl)acetamide | |
| 530 | 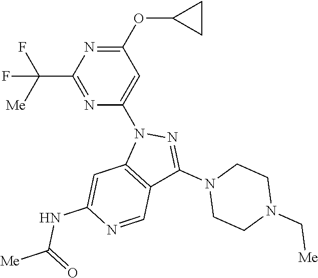 [SMILES: CCN1CCN(c2nn(-c3cc(OC4CC4)nc(C(C)(F)F)n3)c3cc(NC(C)=O)ncc23)CC1] | LCMS m/z = 487.2 [M + H]⁺ 1H NMR (500 MHz, CDCl₃) δ ppm 9.52 (br s, 1H), 8.74 (br s, 1H), 8.18 (br s, 1H), 7.22 (s, 1H), 4.41 (br s, 1H), 3.77 (br s, 4H), 2.82 (br s, 4H), 2.65 (br s, 2H), 2.19-2.33 (m, 6H), 1.19-1.32 (m, 3H), 0.89 (br s, 4H) |
| | N-(1-(6-cyclopropoxy-2-(1,1- | |
| | difluoroethyl)pyrimidin-4-yl)-3-(4- | |
| | ethylpiperazin-1-y1)-1H-pyrazolo[4,3- | |
| | c]pyridin-6-yl)acetamide | |
| 531 | 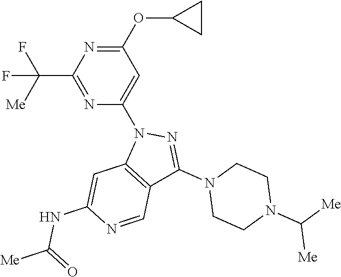 [SMILES: CC(=O)Nc1cc2c(cn1)c(N1CCN(C(C)C)CC1)nn2-c1cc(OC2CC2)nc(C(C)(F)F)n1] | LCMS m/z = 501.2 [M + H]⁺ 1H NMR (500 MHz, CDCl₃) δ ppm 9.51 (br s, 1H), 8.74 (br s, 1H), 8.21 (br s, 1H), 7.21 (s, 1H), 4.36-4.42 (m, 1H), 3.71 (br s, 4H), 2.82 (br s, 5H), 2.62 (s, 1H), 2.20-2.32 (m, 6H), 1.17 (br d, J = 5.5 Hz, 6H), 0.87 (br s, 4H). |
| | N-(1-(6-cyclopropoxy-2-(1,1- | |
| | difluoroethyl)pyrimidin-4-yl)-3-(4- | |
| | isopropylpiperazin-1-yl)-1H-pyrazolo[4,3- | |
| | c]pyridin-6-yl)acetamide | |
| 532 | 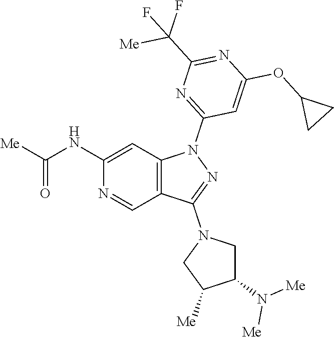 [SMILES: CC(=O)Nc1cc2c(cn1)c(N1C[C@@H](C)[C@@H](N(C)C)C1)nn2-c1cc(OC2CC2)nc(C(C)(F)F)n1] | LCMS m/z = 501.4 [M + H]+ 1H NMR (500 MHz, CDCl₃) δ ppm 9.51 (br s, 1H), 8.69 (br s, 1H), 8.12 (br s, 1H), 7.21 (s, 1H), 4.42 (br s, 1H), 3.83-4.14 (m, 3H), 3.61-3.82 (m, 1H), 2.67- 2.88 (m, 3H), 2.21-2.34 (m, 7 H), 1.60 (m, 4H), 0.90 (br s, 4H). |
| | Or | |
| | 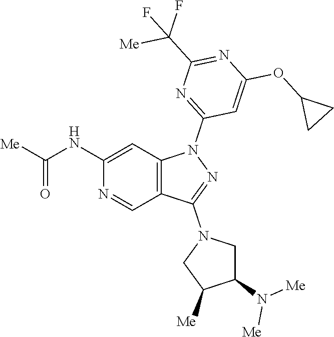 [SMILES: CC(=O)Nc1cc2c(cn1)c(N1C[C@@H](N(C)C)[C@@H](C)C1)nn2-c1cc(OC2CC2)nc(C(C)(F)F)n1] | |
| | N-(1-(6-cyclopropoxy-2-(1,1- | |
| | difluoroethyl)pyrimidin-4-y1)-3-((3R,4R)- | |
| | 3-(dimethylamino)-4-methylpyrrolidin-1- | |
| | y1)-1H-pyrazolo[4,3-c]pyridin-6- | |
| | yl)acetamide or N-(1-(6-cyclopropoxy-2- | |
| | (1,1-difluoroethyl)pyrimidin-4-y1)-3- | |
| | ((3S,4S)-3-(dimethylamino)-4- | |
| | methylpyrrolidin-1-yl)-1H-pyrazolo[4,3- | |
| | c]pyridin-6-yl)acetamide | |
| 533 | 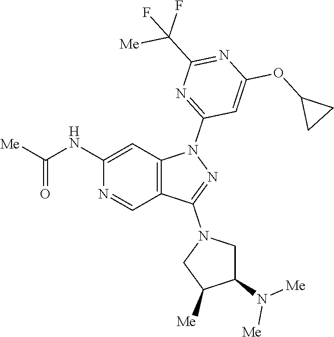 [SMILES: CC(=O)Nc1cc2c(cn1)c(N1C[C@@H](N(C)C)[C@@H](C)C1)nn2-c1cc(OC2CC2)nc(C(C)(F)F)n1] | LCMS m/z = 501.4 [M + H]⁺ 1H NMR (500 MHz, CDCl₃) δ ppm 9.51 (br s, 1H), 8.69 (br s, 1H), 8.12 (br s, 1H), 7.21 (s, 1H), 4.42 (br s, 1H), 3.83-4.14 (m, 3H), 3.61-3.82 (m, 1H), 2.67- 2.88 (m, 3H), 2.21-2.34 (m, 7 H), 1.60 (m, 4H), 0.90 (br s, 4H). |
| | Or | |
| | 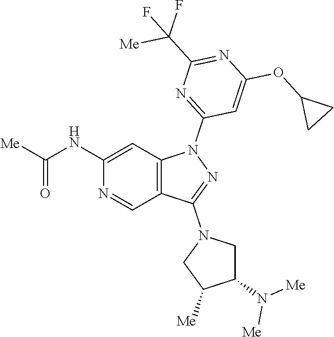 [SMILES: CC(=O)Nc1cc2c(cn1)c(N1C[C@@H](C)[C@@H](N(C)C)C1)nn2-c1cc(OC2CC2)nc(C(C)(F)F)n1] | |
| | N-(1-(6-cyclopropoxy-2-(1,1- | |
| | difluoroethyl)pyrimidin-4-yl)-3-((3S,4S)- | |
| | 3-(dimethylamino)-4-methylpyrrolidin-1- | |
| | yl)-1H-pyrazolo[4,3-c]pyridin-6- | |
| | yl)acetamide or N-(1-(6-cyclopropoxy-2- | |
| | (1,1-difluoroethyl)pyrimidin-4-y1)-3- | |
| | ((3R,4R)-3-(dimethylamino)-4- | |
| | methylpyrrolidin-1-yl)-1H-pyrazolo[4,3- | |
| | c]pyridin-6-yl)acetamide | |
| 534 | 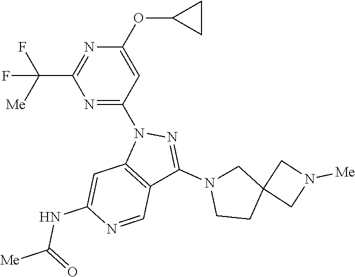 [SMILES: CC(=O)Nc1cc2c(cn1)c(N1CCC3(CN(C)C3)C1)nn2-c1cc(OC2CC2)nc(C(C)(F)F)n1] | LCMS m/z = 499.2 [M + H]⁺ 1H NMR (500 MHz, CDCl₃) δ ppm 9.39 (br s, 1H), 8.66 (s, 1H), 8.54 (br s, 1H), 7.15 (s, 1H), 4.29- 4.40 (m, 1H), 3.85 (s, 2H), 3.69 (br t, J = 6.5 Hz, 3H), 3.37-3.55 (m, 4H), 2.49 (s, 3H), 2.18-2.32 (m, 8 H), 0.86 (br s, 4H) |
| | N-(1-(6-cyclopropoxy-2-(1,1- | |
| | difluoroethyl)pyrimidin-4-yl)-3-(2- | |
| | methyl-2,6-diazaspiro[3.4]octan-6-y1)-1H- | |
| | pyrazolo[4,3-c]pyridin-6-yl)acetamide | |
| 535 | 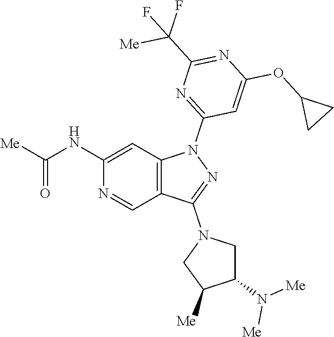 [SMILES: CC(=O)Nc1cc2c(cn1)c(N1C[C@H](C)[C@@H](N(C)C)C1)nn2-c1cc(OC2CC2)nc(C(C)(F)F)n1] | LCMS m/z = 501.3 [M + H]⁺ 1H NMR (500 MHz, CDCl₃) δ ppm 9.48 (br s, 1H), 8.72 (s, 1H), 8.21 (br s, 1H), 7.22 (s, 1H), 4.41 (br s, 1H), 4.03 (br t, J = 8.5 Hz, 1H), 3.82-3.90 (m, 1H), 3.75- 3.82 (m, 1H), 3.35 (br t, J = 8.3 Hz, 1H), 3.00-3.14 (m, 1H), 2.55- 2.66 (m, 1H), 2.50 (br s, 6H), 2.19 -2.35 (m, 6H), 1.27 (br d, J = 6.6 Hz, 3H), 0.88 (br s, 4H). |
| | Or | |
| | 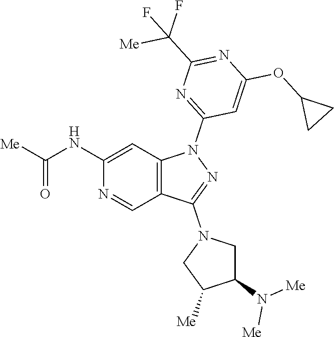 [SMILES: CC(=O)Nc1cc2c(cn1)c(N1C[C@@H](C)[C@H](N(C)C)C1)nn2-c1cc(OC2CC2)nc(C(C)(F)F)n1] | |
| | N-(1-(6-cyclopropoxy-2-(1,1- | |
| | difluoroethyl)pyrimidin-4-yl)-3-((3R,4S)- | |
| | 3-(dimethylamino)-4-methylpyrrolidin-1- | |
| | yl)-1H-pyrazolo[4,3-c]pyridin-6- | |
| | yl)acetamide or N-(1-(6-cyclopropoxy-2- | |
| | (1,1-difluoroethyl)pyrimidin-4-yl)-3- | |
| | ((3S,4R)-3-(dimethylamino)-4- | |
| | methylpyrrolidin-1-yl)-1H-pyrazolo[4,3- | |
| | c]pyridin-6-yl)acetamide | |
| 536 | 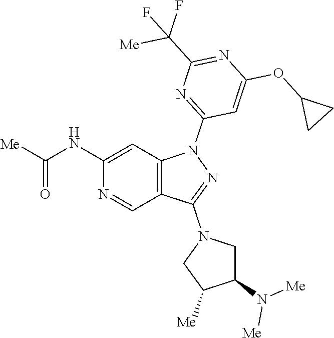 [SMILES: CC(=O)Nc1cc2c(cn1)c(N1C[C@@H](C)[C@H](N(C)C)C1)nn2-c1cc(OC2CC2)nc(C(C)(F)F)n1] | LCMS m/z = 501.3 [M + H]⁺ 1H NMR (500 MHz, CDCl₃) δ ppm 9.48 (br s, 1H), 8.72 (s, 1H), 8.21 (br s, 1H), 7.22 (s, 1H), 4.41 (br s, 1H), 4.03 (br t, J = 8.5 Hz, 1H), 3.82-3.90 (m, 1H), 3.75- 3.82 (m, 1H), 3.35 (br t, J = 8.3 Hz, 1H), 3.00-3.14 (m, 1H), 2.55- 2.66 (m, 1H), 2.50 (br s, 6H), 2.19- 2.35 (m, 6H), 1.27 (br d, J = 6.6 Hz, 3H), 0.88 (br s, 4H). |
| | Or | |
| | 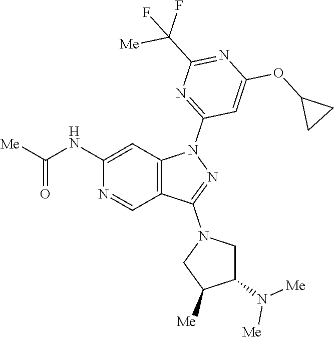 [SMILES: CC(=O)Nc1cc2c(cn1)c(N1C[C@H](C)[C@@H](N(C)C)C1)nn2-c1cc(OC2CC2)nc(C(C)(F)F)n1] | |
| | N-(1-(6-cyclopropoxy-2-(1,1- | |
| | difluoroethyl)pyrimidin-4-yl)-3-((3S,4R)- | |
| | 3-(dimethylamino)-4-methylpyrrolidin-1- | |
| | yl)-1H-pyrazolo[4,3-c]pyridin-6- | |
| | yl)acetamide or N-(1-(6-cyclopropoxy-2- | |
| | (1,1-difluoroethyl)pyrimidin-4-yl)-3- | |
| | ((3R,4S)-3-(dimethylamino)-4- | |
| | methylpyrrolidin-1-yl)-1H-pyrazolo[4,3- | |
| | c]pyridin-6-yl)acetamide | |
| 537 | 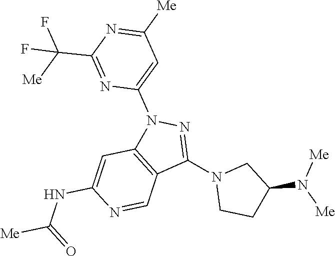 [SMILES: CC(=O)Nc1cc2c(cn1)c(N1CC[C@H](N(C)C)C1)nn2-c1cc(C)nc(C(C)(F)F)n1] | LCMS m/z = 445.2 [M + H]⁺ ¹H NMR: (400 MHz, CDCl₃) δ: ppm 9.47 (s, 1H), 8.72 (s, 1H), 8.05 (s, 1H), 7.64 (s, 1H), 3.93-3.99 (m, 2H), 3.75-3.78 (m, 1H), 3.55-3.65 (m, 1H), 2.95-2.99 (m, 1H), 2.64 (s, 3H), 2.37 (s, 6H), 2.24-2.33 (m, 7H), 2.07-2.09 (m, 1H). |
| | (S)-N-(1-(2-(1,1-difluoroethyl)-6- | |
| | methylpyrimidin-4-y1)-3-(3- | |
| | (dimethylamino)pyrrolidin-1-yl)-1H- | |
| | pyrazolo[4,3-c]pyridin-6-yl)acetamide | |
| 538 | 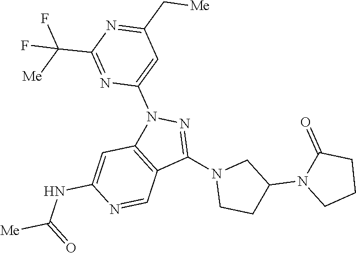 [SMILES: CCc1cc(-n2nc(N3CCC(N4CCCC4=O)C3)c3cnc(NC(C)=O)cc32)nc(C(C)(F)F)n1] | LCMS m/z = 499.2 [M + H]⁺ . ¹H NMR (600 MHz, DMSO-d₆) δ ppm 10.75 (s, 1 H), 9.37 (s, 1 H), 8.95 (s, 1 H), 7.66 (s, 1 H), 4.78 (m, 1 H), 3.89-3.95 (m, 1 H), 3.87 (m, 1 H), 3.74-3.80 (m, 1 H), 3.70 (m, 1 H), 3.42 (t, J = 7.1 Hz, 2 H), 2.85 (m, 2 H), 2.16-2.30 (m, 7 H), 2.15 (s, 3 H), 1.90-1.98 (m, 2 H), 1.28 (t, J = 7.6 Hz, 3 H). |
| | N-(1-(2-(1,1-difluoroethyl)-6- | |
| | ethylpyrimidin-4-yl)-3-(2-oxo-[1,3′- | |
| | bipyrrolidin]-1′-yl)-1H-pyrazolo[4,3- | |
| | c]pyridin-6-yl)acetamide | |
| 539 | 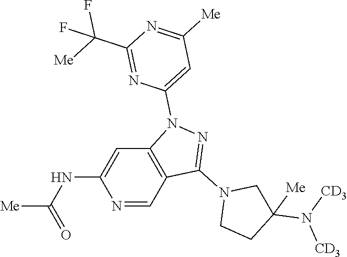 [SMILES: [2H]C([2H])([2H])N(C([2H])([2H])[2H])C1(C)CCN(c2nn(-c3cc(C)nc(C(C)(F)F)n3)c3cc(NC(C)=O)ncc23)C1] | LCMS m/z = 465.2 [M + H]⁺. ¹H NMR (600 MHz, DMSO-d₆) δ ppm 10.71 (s, 1 H), 9.36 (s, 1 H), 8.95 (s, 1 H), 7.67 (s, 1 H), 3.92 (br t, J = 8.8 Hz, 1 H), 3.76-3.84 (m, 1 H), 3.73 (br d, J = 9.2 Hz, 1 H), 3.59 (br d, J = 6.9 Hz, 1 H), 2.57 (s, 3 H), 2.21 (t, J = 19.3 Hz, 3 H), 2.15 (s, 3 H), 1.99-2.11 (m, 2 H), 1.15 (br s, 3 H). |
| | N-(3-(3-(bis(methyl-d3)amino)-3- | |
| | methylpyrrolidin-1-y1)-1-(2-(1,1- | |
| | difluoroethyl)-6-methylpyrimidin-4-yl)- | |
| | 1H-pyrazolo[4,3-c]pyridin-6-yl)acetamide | |
| 540 | 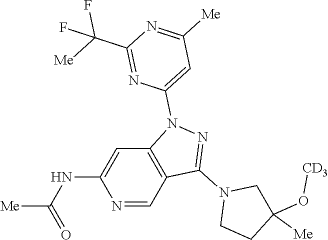 [SMILES: [2H]C([2H])([2H])OC1(C)CCN(c2nn(-c3cc(C)nc(C(C)(F)F)n3)c3cc(NC(C)=O)ncc23)C1] | LCMS m/z = 449.1 [M + H]⁺ ¹H NMR (600 MHz, DMSO-d₆) δ ppm 10.73 (s, 1 H), 9.36 (s, 1 H), 8.93 (d, J = 0.8 Hz, 1 H), 7.66 (s, 1 H), 3.72-3.87 (m, 4 H), 2.53- 2.58 (m, 3 H), 2.17-2.27 (m, 4 H), 2.15 (s, 3 H), 1.89-2.00 (m, 1 H), 1.41 (s, 3 H). |
| | N-(1-(2-(1,1-difluoroethyl)-6- | |
| | methylpyrimidin-4-yl)-3-(3-(methoxy- | |
| | d3)-3-methylpyrrolidin-1-yl)-1H- | |
| | pyrazolo[4,3-c]pyridin-6-yl)acetamide | |
| 541 | 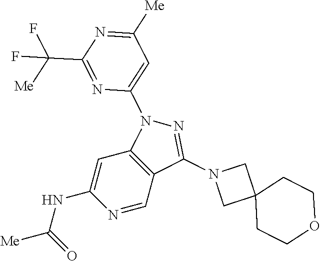 [SMILES: CC(=O)Nc1cc2c(cn1)c(N1CC3(CCOCC3)C1)nn2-c1cc(C)nc(C(C)(F)F)n1] | LCMS m/z = 458.1 [M + H]⁺ ¹H NMR (600 MHz, DMSO-d₆) δ ppm 10.75 (s, 1 H), 9.34 (s, 1 H), 8.76 (s, 1 H), 7.63 (s, 1 H), 4.09 (s, 4 H), 3.54-3.63 (m, 4 H), 2.57 (s, 3 H), 2.21 (t, J = 19.3 Hz, 3 H), 2.15 (s, 3 H), 1.82 (br t, J = 5.0 Hz, 4 H). |
| | N-(1-(2-(1,1-difluoroethyl)-6- | |
| | methylpyrimidin-4-yl)-3-(7-oxa-2- | |
| | azaspiro[3.5]nonan-2-y1)-1H- | |
| | pyrazolo[4,3-c]pyridin-6-yl)acetamide | |
| 542 | 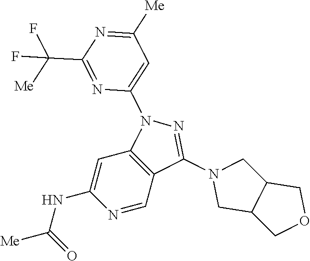 [SMILES: CC(=O)Nc1cc2c(cn1)c(N1CC3COCC3C1)nn2-c1cc(C)nc(C(C)(F)F)n1] | LCMS m/z = 444.1 [M + H]⁺ ¹H NMR (600 MHz, DMSO-d₆) δ ppm 10.75 (s, 1 H), 9.37 (s, 1 H), 8.97 (s, 1 H), 7.67 (s, 1 H), 3.83- 3.92 (m, 4 H), 3.62-3.71 (m, 4 H), 3.10 (br d, J = 3.1 Hz, 2 H), 2.57 (s, 3 H), 2.22 (t, J = 19.3 Hz, 3 H), 2.15 (s, 3 H). |
| | N-(1-(2-(1,1-difluoroethyl)-6- | |
| | methylpyrimidin-4-yl)-3-(tetrahydro-1H- | |
| | furo[3,4-c]pyrrol-5(3H)-y1)-1H- | |
| | pyrazolo[4,3-c]pyridin-6-yl)acetamide | |
| 543 | 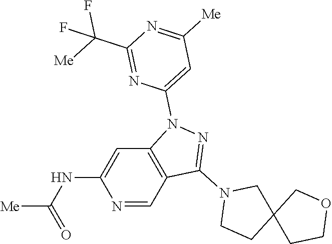 [SMILES: CC(=O)Nc1cc2c(cn1)c(N1CCC3(CCOC3)C1)nn2-c1cc(C)nc(C(C)(F)F)n1] | LCMS m/z = 458.1 [M + H]⁺. ¹H NMR (600 MHz, DMSO-d₆) δ ppm 10.74 (s, 1 H), 9.36 (s, 1 H), 8.93 (s, 1 H), 7.66 (s, 1 H), 3.75- 3.90 (m, 4 H), 3.59-3.72 (m, 4 H), 2.56 (s, 3 H), 2.21 (t, J = 19.1 Hz, 3 H), 2.15 (s, 3 H), 1.90-2.10 (m, 4 H). |
| | N-(1-(2-(1,1-difluoroethyl)-6- | |
| | methylpyrimidin-4-yl)-3-(2-oxa-7- | |
| | azaspiro[4.4]nonan-7-y1)-1H- | |
| | pyrazolo[4,3-c]pyridin-6-yl)acetamide | |
| 544 | 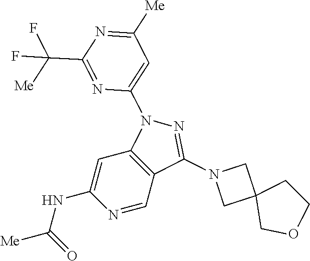 [SMILES: CC(=O)Nc1cc2c(cn1)c(N1CC3(CCOC3)C1)nn2-c1cc(C)nc(C(C)(F)F)n1] | LCMS m/z = 444.1 [M + H]⁺. ¹H NMR (600 MHz, DMSO-d₆) δ ppm 10.75 (s, 1 H), 9.34 (s, 1 H), 8.75 (s, 1 H), 7.64 (s, 1 H), 4.29 (s, 4 H), 3.89 (s, 2 H), 3.76 (t, J = 7.1 Hz, 2 H), 2.58 (s, 3 H), 2.17-2.26 (m, 5 H), 2.15 (s, 3 H). |
| | N-(1-(2-(1,1-difluoroethyl)-6- | |
| | methylpyrimidin-4-yl)-3-(6-oxa-2- | |
| | azaspiro[3.4]octan-2-y1)-1H-pyrazolo[4,3- | |
| | c]pyridin-6-yl)acetamide | |
| 545 | 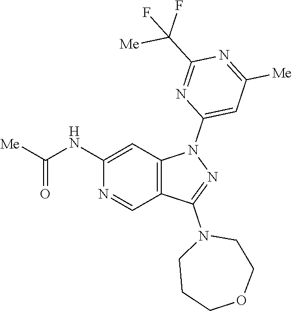 [SMILES: CC(=O)Nc1cc2c(cn1)c(N1CCCOCC1)nn2-c1cc(C)nc(C(C)(F)F)n1] | LCMS m/z = 432.3 [M + H]⁺. ¹H NMR (600 MHz, DMSO-d₆) δ ppm 10.72 (s, 1 H), 9.39 (s, 1 H), 8.97 (d, J = 0.8 Hz, 1 H), 7.68 (s, 1 H), 3.90-3.96 (m, 4 H), 3.84- 3.89 (m, 2 H), 3.67-3.73 (m, 2 H), 2.57 (s, 3 H), 2.17-2.27 (m, 3 H), 2.15 (s, 3 H), 1.98-2.05 (m, 2 H). |
| | N-(1-(2-(1,1-difluoroethyl)-6- | |
| | methylpyrimidin-4-yl)-3-(1,4-oxazepan-4- | |
| | yl)-1H-pyrazolo[4,3-c]pyridin-6- | |
| | yl)acetamide | |
| 546 | 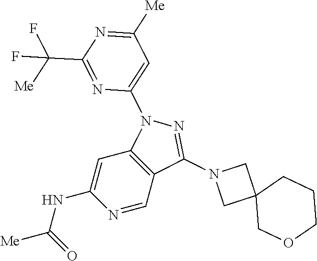 [SMILES: CC(=O)Nc1cc2c(cn1)c(N1CC3(CCCOC3)C1)nn2-c1cc(C)nc(C(C)(F)F)n1] | LCMS m/z = 458.3 [M + H]⁺. ¹H NMR (600 MHz, DMSO-d₆) δ ppm 10.75 (s, 1 H), 9.33 (d, J = 0.8 Hz, 1 H), 8.77 (d, J = 1.1 Hz, 1 H), 7.63 (s, 1 H), 4.04 (d, J = 7.6 Hz, 2 H), 3.98 (d, J = 7.6 Hz, 2 H), 3.71 (s, 2 H), 3.52-3.57 (m, 2 H), 2.57 (s, 3 H), 2.17-2.25 (m, 3 H), 2.14 (s, 3 H), 1.86-1.90 (m, 2 H), 1.52- 1.58 (m, 2 H). |
| | N-(1-(2-(1,1-difluoroethyl)-6- | |
| | methylpyrimidin-4-yl)-3-(6-oxa-2- | |
| | azaspiro[3.5]nonan-2-yl)-1H- | |
| | pyrazolo[4,3-c]pyridin-6-yl)acetamide | |
| 547 | 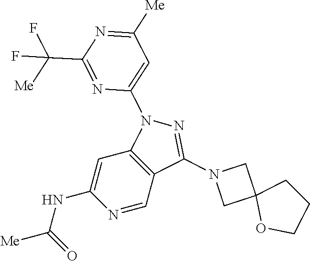 [SMILES: CC(=O)Nc1cc2c(cn1)c(N1CC3(CCCO3)C1)nn2-c1cc(C)nc(C(C)(F)F)n1] | LCMS m/z = 444.3 [M + H]⁺. ¹H NMR (600 MHz, DMSO-d₆) δ ppm 10.74 (s, 1 H), 9.33 (s, 1 H), 8.73 (d, J = 0.8 Hz, 1 H), 7.63 (s, 1 H), 4.34 (d, J = 8.8 Hz, 2 H), 4.26 (d, J = 8.8 Hz, 2 H), 3.81 (t, J = 6.9 Hz, 2 H), 2.57 (s, 3 H), 2.16-2.25 (m, 5 H), 2.15 (s, 3 H), 1.91 (m, 2 H). |
| | N-(1-(2-(1,1-difluoroethyl)-6- | |
| | methylpyrimidin-4-yl)-3-(5-oxa-2- | |
| | azaspiro[3.4]octan-2-yl)-1H-pyrazolo[4,3- | |
| | c]pyridin-6-yl)acetamide | |
| 548 | 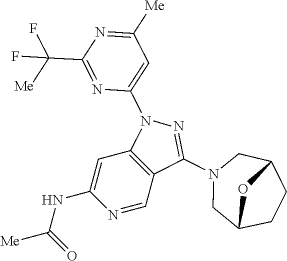 [SMILES: CC(=O)Nc1cc2c(cn1)c(N1C[C@H]3CC[C@@H](C1)O3)nn2-c1cc(C)nc(C(C)(F)F)n1] | LCMS m/z = 444.3 [M + H]⁺. ¹H NMR (600 MHz, DMSO-d₆) δ ppm 10.77 (s, 1 H), 9.40 (s, 1 H), 9.06 (d, J = 0.8 Hz, 1 H), 7.70 (s, 1 H), 4.49 (br d, J = 2.3 Hz, 2 H), 3.88 (d, J = 11.8 Hz, 2 H), 3.29- 3.36 (m, 2 H), 2.57 (s, 3 H), 2.18- 2.26 (m, 3 H), 2.15 (s, 3 H), 1.88- 1.99 (m, 4 H). |
| | N-(3-((1R,5S)-8-oxa-3- | |
| | azabicyclo[3.2.1]octan-3-y1)-1-(2-(1,1- | |
| | difluoroethyl)-6-methylpyrimidin-4-yl)- | |
| | 1H-pyrazolo[4,3-c]pyridin-6-yl)acetamide | |
| 549 | 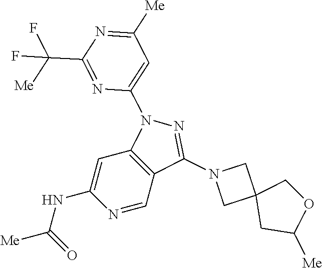 [SMILES: CC(=O)Nc1cc2c(cn1)c(N1CC3(COC(C)C3)C1)nn2-c1cc(C)nc(C(C)(F)F)n1] | LCMS m/z = 458.3 [M + H]⁺. ¹H NMR (600 MHz, DMSO-d₆) δ ppm 10.75 (s, 1 H), 9.34 (d, J = 0.8 Hz, 1 H), 8.75 (d, J = 1.1 Hz, 1 H), 7.63 (s, 1 H), 4.24-4.33 (m, 4 H), 4.04 (d, J = 9.2 Hz, 1 H), 3.95-4.02 (m, 1 H), 3.85 (d, J = 9.2 Hz, 1 H), 2.57 (s, 3 H), 2.40 (m, 1 H), 2.21 (t, J = 19.3 Hz, 3 H), 2.15 (s, 3 H), 1.79 (m, 1 H), 1.19 (d, J = 6.1 Hz, 3 H). |
| | N-(1-(2-(1,1-difluoroethyl)-6- | |
| | methylpyrimidin-4-yl)-3-(7-methyl-6-oxa- | |
| | 2-azaspiro[3.4]octan-2-y1)-1H- | |
| | pyrazolo[4,3-c]pyridin-6-yl)acetamide | |
| 550 | 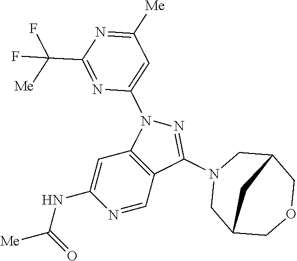 [SMILES: CC(=O)Nc1cc2c(cn1)c(N1C[C@@H]3COC[C@@H](C3)C1)nn2-c1cc(C)nc(C(C)(F)F)n1] | LCMS m/z = 458.3 [M + H]⁺. |
| | N-(3-((1R,5S)-3-oxa-7- | |
| | azabicyclo[3.3.1]nonan-7-y1)-1-(2-(1,1- | |
| | difluoroethyl)-6-methylpyrimidin-4-yl)- | |
| | 1H-pyrazolo[4,3-c]pyridin-6-yl)acetamide | |
| 551 | 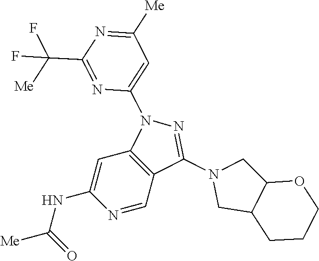 [SMILES: CC(=O)Nc1cc2c(cn1)c(N1CC3CCCOC3C1)nn2-c1cc(C)nc(C(C)(F)F)n1] | LCMS m/z = 458.3 [M + H]⁺. ¹H NMR (600 MHz, DMSO-d₆) δ ppm 10.67-10.78 (m, 1 H), 9.31- 9.39 (m, 1 H), 8.88-8.95 (m, 1 H), 7.61-7.69 (m, 1 H), 3.90-4.14 (m, 2 H), 3.66-3.88 (m, 4 H), 3.27- 3.59 (m, 4 H), 2.55-2.57 (m, 3 H), 2.41-2.48 (m, 1 H), 2.16- 2.26 (m, 3 H), 2.13-2.16 (m, 3 H), 1.84-1.89 (m, 1 H). |
| | N-(1-(2-(1,1-difluoroethyl)-6- | |
| | methylpyrimidin-4-y1)-3- | |
| | (hexahydropyrano[2,3-c]pyrrol-6(2H)-y1)- | |
| | 1H-pyrazolo[4,3-c]pyridin-6-yl)acetamide | |
| 552 | 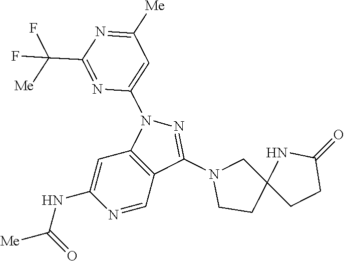 [SMILES: CC(=O)Nc1cc2c(cn1)c(N1CCC3(CCC(=O)N3)C1)nn2-c1cc(C)nc(C(C)(F)F)n1] | LCMS m/z = 471.1 [M + H]⁺. 1H NMR (600 MHz, DMSO-d₆) δ ppm 10.74 (s, 1 H), 9.37 (s, 1 H), 8.95 (s, 1 H), 8.17 (s, 1 H), 7.68 (s, 1 H), 3.87-3.94 (m, 1 H), 3.80- 3.86 (m, 1 H), 3.78 (br d, J = 9.9 Hz, 1 H), 3.68 (d, J = 9.9 Hz, 1 H), 2.57 (s, 3 H), 2.26-2.39 (m, 2 H), 2.22 (br t, J = 19.3 Hz, 3 H), 2.15 (s, 3 H), 2.06-2.14 (m, 4 H). |
| | N-(1-(2-(1,1-difluoroethyl)-6- | |
| | methylpyrimidin-4-yl)-3-(2-oxo-1,7- | |
| | diazaspiro[4.4]nonan-7-y1)-1H- | |
| | pyrazolo[4,3-c]pyridin-6-yl)acetamide | |
| 553 | 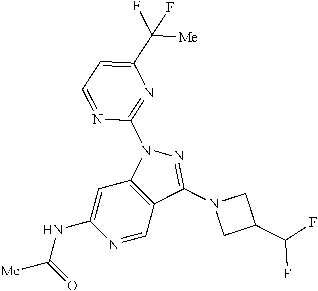 [SMILES: CC(=O)Nc1cc2c(cn1)c(N1CC(C(F)F)C1)nn2-c1nccc(C(C)(F)F)n1] | LCMS m/z = 424 [M + H]⁺. 1H NMR (DMSO-d₆, 600 MHz) δ 10.70 (s, 1H), 9.26 (s, 1H), 9.00- 9.10 (m, 1H), 8.78 (s, 1H), 7.50- 7.60 (m, 1H), 6.43 (dt, 1H, J = 4.0, 56.5 Hz), 4.40 (t, 2H, J = 8.4 Hz), 4.20-4.30 (m, 2H), 3.30-3.32 (m, 1H), 2.20-2.30 (m, 3H), 2.15 (s, 3H). |
| | N-(1-(4-(1,1-difluoroethyl)pyrimidin-2- | |
| | y1)-3-(3-(difluoromethyl)azetidin-1-yl)- | |
| | 1H-pyrazolo[4,3-c]pyridin-6-yl)acetamide | |
| 554 | 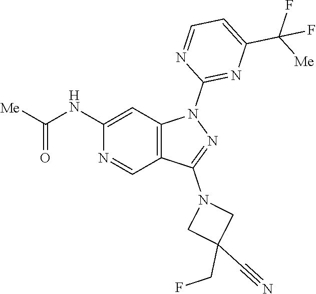 [SMILES: CC(=O)Nc1cc2c(cn1)c(N1CC(C#N)(CF)C1)nn2-c1nccc(C(C)(F)F)n1] | LCMS m/z = 431 [M + H]⁺. 1H NMR (DMSO-d₆, 600 MHz) δ 10.72 (s, 1H), 9.27 (s, 1H), 9.00- 9.10 (m, 1H), 8.80 (s, 1H), 7.57 (d, 1H, J = 5.1 Hz), 4.90-5.00 (m, 2H), 4.64 (d, 2H, J = 8.7 Hz), 4.44 (d, 2H, J = 8.7 Hz), 2.21 (t, 3H, J = 19.6 Hz), 2.15 (s, 3H). |
| | N-(3-(3-cyano-3-(fluoromethyl)azetidin- | |
| | 1-y1)-1-(4-(1,1-difluoroethyl)pyrimidin-2- | |
| | y1)-1H-pyrazolo[4,3-c]pyridin-6- | |
| | yl)acetamide | |
| 555 | 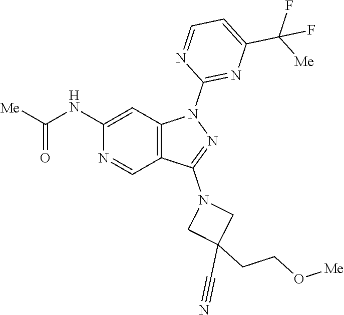 [SMILES: COCCC1(C#N)CN(c2nn(-c3nccc(C(C)(F)F)n3)c3cc(NC(C)=O)ncc23)C1] | LCMS m/z = 457 [M + H]⁺ |
| | N-(3-(3-cyano-3-(2- | |
| | methoxyethyl)azetidin-1-y1)-1-(4-(1,1- | |
| | difluoroethyl)pyrimidin-2-yl)-1H- | |
| | pyrazolo[4,3-c]pyridin-6-yl)acetamide | |
| 556 | 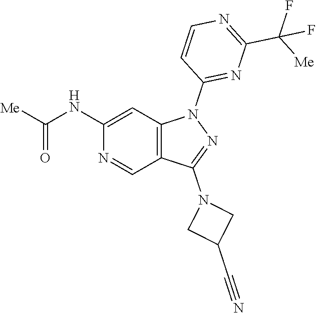 [SMILES: CC(=O)Nc1cc2c(cn1)c(N1CC(C#N)C1)nn2-c1ccnc(C(C)(F)F)n1] | LCMS m/z = 399 [M + H]⁺ 1H NMR (DMSO-d₆, 500 MHz) δ 10.7-10.8 (m, 1H), 9.28 (s, 1H), 9.05 (d, 1H, J = 5.0 Hz), 8.79 (d, 1H, J = 0.9 Hz), 7.58 (d, 1H, J = 5.0 Hz), 4.5-4.6 (m, 2H), 4.45 (dd, 2H, J = 5.6, 7.6 Hz), 4.03 (tt, 1H, J = 5.7, 8.7 Hz), 2.22 (t, 3H, J = 19.6 Hz), 2.15 (s, 3H). |
| | N-(3-(3-cyanoazetidin-1-y1)-1-(2-(1,1- | |
| | difluoroethyl)pyrimidin-4-yl)-1H- | |
| | pyrazolo[4,3-c]pyridin-6-yl)acetamide | |
| 557 | 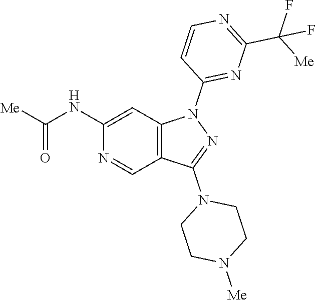 [SMILES: CC(=O)Nc1cc2c(cn1)c(N1CCN(C)CC1)nn2-c1ccnc(C(C)(F)F)n1] | LCMS m/z = 417 [M + H]⁺ |
| | N-(1-(2-(1,1-difluoroethyl)pyrimidin-4- | |
| | yl)-3-(4-methylpiperazin-1-yl)-1H- | |
| | pyrazolo[4,3-c]pyridin-6-yl)acetamide | |
| 558 | 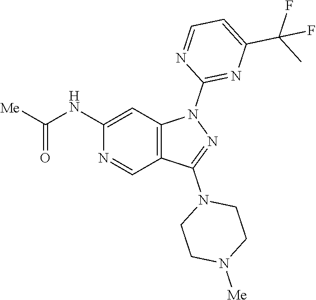 [SMILES: CC(=O)Nc1cc2c(cn1)c(N1CCN(C)CC1)nn2-c1nccc(C(C)(F)F)n1] | LCMS m/z = 417 [M + H]⁺ |
| | N-(1-(4-(1,1-difluoroethyl)pyrimidin-2- | |
| | yl)-3-(4-methylpiperazin-1-yl)-1H- | |
| | pyrazolo[4,3-c]pyridin-6-yl)acetamide | |
| 559 | 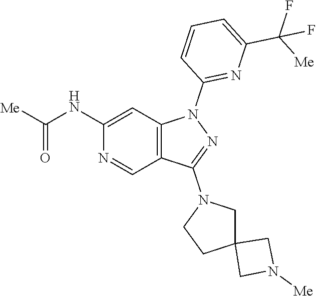 [SMILES: CC(=O)Nc1cc2c(cn1)c(N1CCC3(CN(C)C3)C1)nn2-c1cccc(C(C)(F)F)n1] | LCMS m/z = 442.2 [M + H]⁺ 1H NMR (500 MHz, CDCl₃) δ ppm 9.38 (br s, 1H), 8.72 (br s, 1H), 8.33 (br s, 1H), 7.92 (br d, J = 7.9 Hz, 1H), 7.87 (br t, J = 7.7 Hz, 1H), 7.40-7.47 (m, 1H), 7.28-7.28 (m, 1H), 3.88 (br s, 2H), 3.75 (br t, J = 5.9 Hz, 2H), 3.45 (br s, 4H), 2.49 (br s, 3H), 2.22-2.38 (m, 8 H). |
| | N-(1-(6-(1,1-difluoroethyl)pyridin-2-yl)- | |
| | 3-(2-methyl-2,6-diazaspiro[3.4]octan-6- | |
| | yl)-1H-pyrazolo[4,3-c]pyridin-6- | |
| | yl)acetamide | |
| 560 | 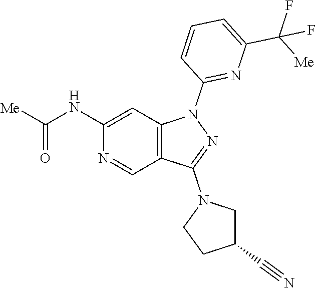 [SMILES: CC(=O)Nc1cc2c(cn1)c(N1CC[C@@H](C#N)C1)nn2-c1cccc(C(C)(F)F)n1] | LCMS m/z = 412 [M + H]⁺ 1H NMR (500 MHz, DMSO-d₆) δ 10.71 (s, 1H), 9.32 (s, 1H), 8.96 (d, J = 0.76 Hz, 1H), 8.05-8.15 (m, 1H), 7.94 (d, J = 8.24 Hz, 1H), 7.45-7.56 (m, 1H), 3.98-4.06 (m, 1H), 3.84- 3.96 (m, 2H), 3.75-3.83 (m, 1H), 3.60-3.71 (m, 1H), 2.41-2.48 (m, 1H), 2.31-2.38 (m, 1H), 2.26 (t, J = 19.45 Hz, 3H), 2.08-2.20 (m, 3H). |
| | (R)-N-(3-(3-cyanopyrrolidin-1-y1)-1-(6- | |
| | (1,1-difluoroethyl)pyridin-2-yl)-1H- | |
| | pyrazolo[4,3-c]pyridin-6-yl)acetamide | |
| 561 | 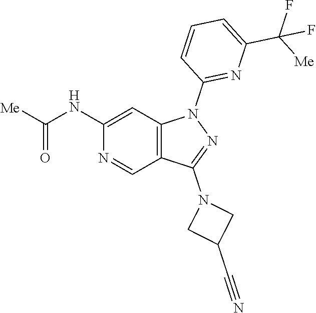 [SMILES: CC(=O)Nc1cc2c(cn1)c(N1CC(C#N)C1)nn2-c1cccc(C(C)(F)F)n1] | LCMS m/z = 412 [M + H]⁺ ¹H NMR (500 MHz, DMSO-d₆) δ 10.71 (s, 1H), 9.32 (s, 1H), 8.96 (d, J = 0.76 Hz, 1H), 8.05-8.15 (m, 1H), 7.94 (d, J = 8.24 Hz, 1H), 7.45-7.56 (m, 1H), 3.98-4.06 (m, 1H), 3.84- 3.96 (m, 2H), 3.75-3.83 (m, 1H), 3.60-3.71 (m, 1H), 2.41-2.48 (m, 1H), 2.31-2.38 (m, 1H), 2.26 (t, J = 19.45 Hz, 3H), 2.08-2.20 (m, 3H). |
| | N-(3-(3-cyanoazetidin-1-yl)-1-(6-(1,1- | |
| | difluoroethyl)pyridin-2-y1)-1H- | |
| | pyrazolo[4,3-c]pyridin-6-yl)acetamide | |
| 562 | 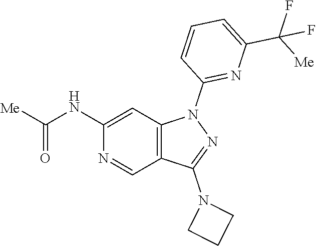 [SMILES: CC(=O)Nc1cc2c(cn1)c(N1CCC1)nn2-c1cccc(C(C)(F)F)n1] | LCMS m/z = 373 [M + H]⁺ ¹H NMR (500 MHz, DMSO-d₆) δ: 10.67 (s, 1H), 9.29 (s, 1H), 8.72 (d, J = 0.92 Hz, 1H), 8.08 (t, J = 7.93 Hz, 1H), 7.89 (d, J = 8.24 Hz, 1H), 7.49 (d, J = 7.02 Hz, 1H), 4.27 (t, J = 7.40 Hz, 4H), 2.44-2.49 (m, 2H), 2.25 (t, J = 19.45 Hz, 3H), 2.15 (s, 3H). |
| | N-(3-(azetidin-1-yl)-1-(6-(1,1- | |
| | difluoroethyl)pyridin-2-yl)-1H- | |
| | pyrazolo[4,3-c]pyridin-6-yl)acetamide | |
| 563 | 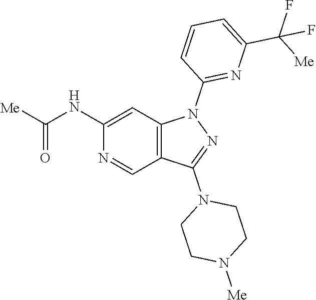 [SMILES: CC(=O)Nc1cc2c(cn1)c(N1CCN(C)CC1)nn2-c1cccc(C(C)(F)F)n1] | LCMS m/z = 416 [M + H]⁺ ¹H NMR (DMSO-d₆, 600 MHz) δ 10.66 (s, 1H), 9.33 (s, 1H), 9.04 (s, 1H), 8.09 (t, 1H, J = 7.6 Hz), 7.94 (br d, 1H, J = 8.0 Hz), 7.50 (d, 1H, J = 7.3 Hz), 3.59 (br s, 4H), 2.54 (br s, 4H), 2.20-2.30 (m, 6H), 2.15 (s, 3H). |
| | N-(1-(6-(1,1-difluoroethyl)pyridin-2-yl)- | |
| | 3-(4-methylpiperazin-1-yl)-1H- | |
| | pyrazolo[4,3-c]pyridin-6-yl)acetamide | |
| 564 | 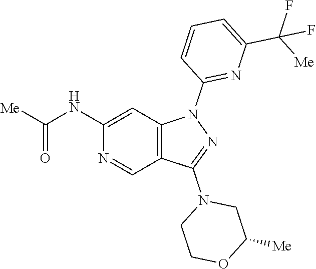 [SMILES: CC(=O)Nc1cc2c(cn1)c(N1CCO[C@@H](C)C1)nn2-c1cccc(C(C)(F)F)n1] | LCMS m/z = 417 [M + H]⁺ ¹H NMR (DMSO-d₆, 600 MHz) δ 10.66 (s, 1H), 9.34 (s, 1H), 9.08 (s, 1H), 8.10 (t, 1H, J = 8.0 Hz), 7.96 (d, 1H, J = 8.7 Hz), 7.51 (d, 1H, J = 7.3 Hz), 3.90-4.00 (m, 3H), 3.70-3.80 (m, 2H), 3.10 (dt, 1H, J = 3.3, 12.2 Hz), 2.70-2.80 (m, 1H), 2.25 (t, 3H, J = 19.3 Hz), 2.15 (s, 3H), 1.22 (d, 3H, J = 5.8 Hz). |
| | (S)-N-(1-(6-(1,1-difluoroethyl)pyridin-2- | |
| | yl)-3-(2-methylmorpholino)-1H- | |
| | pyrazolo[4,3-c]pyridin-6-yl)acetamide | |
| 565 | 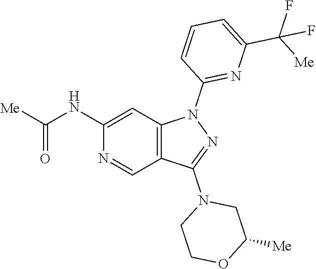 [SMILES: CC(=O)Nc1cc2c(cn1)c(N1CCO[C@@H](C)C1)nn2-c1cccc(C(C)(F)F)n1] | LCMS m/z = 417 [M + H]⁺ ¹H NMR (DMSO-d₆, 600 MHz) δ 10.66 (s, 1H), 9.34 (s, 1H), 9.08 (s, 1H), 8.10 (t, 1H, J = 8.0 Hz), 7.96 (d, 1H, J = 8.0 Hz), 7.51 (d, 1H, J = 7.3 Hz), 4.02 (br d, 1H, J = 12.4 Hz), 3.90-4.00 (m, 2H), 3.70-3.80 (m, 2H), 3.10 (dt, 1H, J = 2.9, 12.0 Hz), 2.70-2.80 (m, 1H), 2.20-2.30 (m, 3H), 2.15 (s, 3H), 1.22 (d, 3H, J = 5.8 Hz). |
| | (R)-N-(1-(6-(1,1-difluoroethyl)pyridin-2- | |
| | yl)-3-(2-methylmorpholino)-1H- | |
| | pyrazolo[4,3-c]pyridin-6-yl)acetamide | |
| 566 | 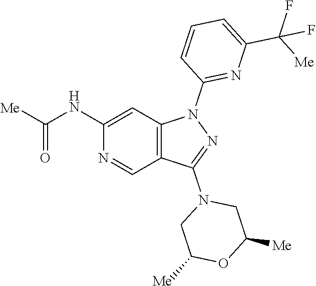 [SMILES: CC(=O)Nc1cc2c(cn1)c(N1C[C@@H](C)O[C@H](C)C1)nn2-c1cccc(C(C)(F)F)n1] | LCMS m/z = 431 [M + H]⁺ |
| | N-(1-(6-(1,1-difluoroethyl)pyridin-2-y1)- | |
| | 3-((2R,6R)-2,6-dimethylmorpholino)-1H- | |
| | pyrazolo[4,3-c]pyridin-6-yl)acetamide | |
| 567 | 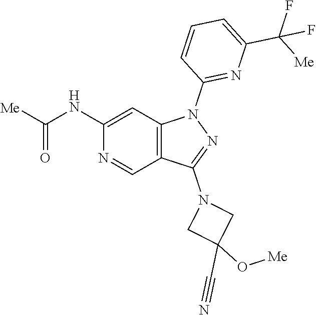 [SMILES: COC1(C#N)CN(c2nn(-c3cccc(C(C)(F)F)n3)c3cc(NC(C)=O)ncc23)C1] | LCMS m/z = 428 [M + H]⁺ |
| | N-(3-(3-cyano-3-methoxyazetidin-1-yl)-1- | |
| | (6-(1,1-difluoroethyl)pyridin-2-yl)-1H- | |
| | pyrazolo[4,3-c]pyridin-6-yl)acetamide | |
| 568 | 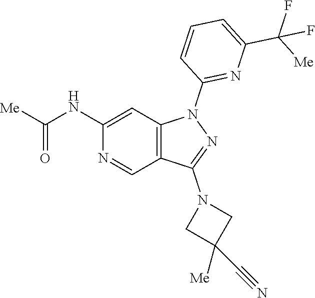 [SMILES: CC(=O)Nc1cc2c(cn1)c(N1CC(C)(C#N)C1)nn2-c1cccc(C(C)(F)F)n1] | LCMS m/z = 412 [M + H]⁺ ¹H NMR (DMSO-d₆, 500 MHz) δ 10.71 (s, 1H), 9.30 (s, 1H), 8.77 (d, 1H, J = 1.1 Hz), 8.10-8.20 (m, 1H), 7.90 (d, 1H, J = 8.2 Hz), 7.52 (dd, 1H, J = 0.6, 7.6 Hz), 4.61 (d, 2H, J = 7.8 Hz), 4.27 (d, 2H, J = 7.9 Hz), 2.25 (t, 3H, J = 19.5 Hz), 2.15 (s, 3H), 1.72 (s, 3H). |
| | N-(3-(3-cyano-3-methylazetidin-1-yl)-1- | |
| | (6-(1,1-difluoroethyl)pyridin-2-y1)-1H- | |
| | pyrazolo[4,3-c]pyridin-6-yl)acetamide | |
| 569 | 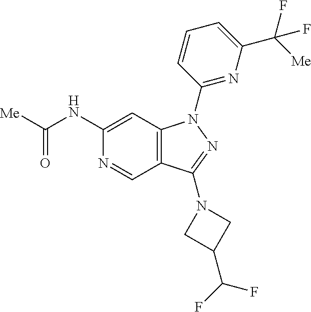 [SMILES: CC(=O)Nc1cc2c(cn1)c(N1CC(C(F)F)C1)nn2-c1cccc(C(C)(F)F)n1] | LCMS m/z = 423 [M + H]⁺ ¹H NMR (DMSO-d₆, 500 MHz) δ 10.70 (s, 1H), 9.30 (s, 1H), 8.79 (s, 1H), 8.10 (t, 1H, J = 8.0 Hz), 7.90 (d, 1H, J = 8.4 Hz), 7.50 (d, 1H, J = 7.6 Hz), 6.30-6.60 (m, 1H), 4.40 (t, 2H, J = 8.3 Hz), 4.23 (dd, 2H, J = 5.8, 7.9 Hz), 4.00-4.10 (m, 1H), 2.20-2.30 (m, 3H), 2.15 (s, 3H). |
| | N-(1-(6-(1,1-difluoroethyl)pyridin-2-y1)- | |
| | 3-(3-(difluoromethyl)azetidin-1-yl)-1H- | |
| | pyrazolo[4,3-c]pyridin-6-yl)acetamide | |
| 570 | 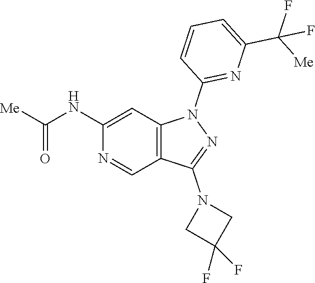 [SMILES: CC(=O)Nc1cc2c(cn1)c(N1CC(F)(F)C1)nn2-c1cccc(C(C)(F)F)n1] | LCMS m/z = 409 [M + H]⁺ 1H NMR (400 MHz, MeOD-d₄) δ 9.11 (s, 1H), 8.72 (s, 1H), 7.96-8.09 (m, 2H), 7.53 (dd, J = 0.88, 7.40 Hz, 1H), 4.70 (t, J = 12.17 Hz, 4H), 2.15-2.30 (m, 6H) |
| | N-(3-(3,3-difluoroazetidin-1-y1)-1-(6-(1,1- | |
| | difluoroethyl)pyridin-2-y1)-1H- | |
| | pyrazolo[4,3-c]pyridin-6-yl)acetamide | |
| 571 | 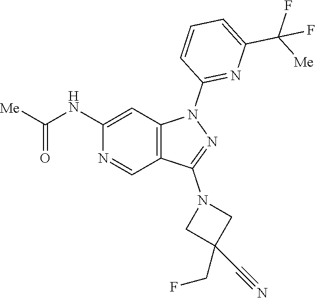 [SMILES: CC(=O)Nc1cc2c(cn1)c(N1CC(C#N)(CF)C1)nn2-c1cccc(C(C)(F)F)n1] | LCMS m/z = 430 [M + H]⁺ ¹H NMR (DMSO-d₆, 500 MHz) δ 10.7-10.8 (m, 1H), 9.31 (s, 1H), 8.80 (d, 1H, J = 0.9 Hz), 8.10-8.20 (m, 1H), 7.91 (d, 1H, J = 8.2 Hz), 7.53 (d, 1H, J = 7.2 Hz), 4.90-5.10 (m, 2H), 4.63 (dd, 2H, J = 2.0, 8.2 Hz), 4.43 (d, 2H, J = 8.2 Hz), 2.25 (t, 3H, J = 19.4 Hz), 2.15 (s, 3H). |
| | N-(3-(3-cyano-3-(fluoromethyl)azetidin- | |
| | 1-y1)-1-(6-(1,1-difluoroethyl)pyridin-2- | |
| | yl)-1H-pyrazolo[4,3-c]pyridin-6- | |
| | yl)acetamide | |
| 572 | 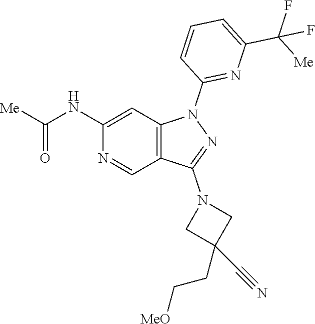 [SMILES: COCCC1(C#N)CN(c2nn(-c3cccc(C(C)(F)F)n3)c3cc(NC(C)=O)ncc23)C1] | LCMS m/z = 456 [M + H]⁺ 1H NMR (DMSO-d₆, 500 MHz) δ 10.72 (s, 1H), 9.30 (s, 1H), 8.78 (s, 1H), 8.11 (t, 1H, J = 7.9 Hz), 7.91 (d, 1H, J = 8.2 Hz), 7.52 (d, 1H, J = 7.5 Hz), 4.58 (d, 2H, J = 7.9 Hz), 4.35 (d, 2H, J = 7.9 Hz), 3.40-3.60 (m, 2H), 3.20-3.30 (m, 3H), 2.20- 2.30 (m, 5H), 2.10-2.20 (m, 3H). |
| | N-(3-(3-cyano-3-(2- | |
| | methoxyethyl)azetidin-1-yl)-1-(6-(1,1- | |
| | difluoroethyl)pyridin-2-y1)-1H- | |
| | pyrazolo[4,3-c]pyridin-6-yl)acetamide | |
| 573 | 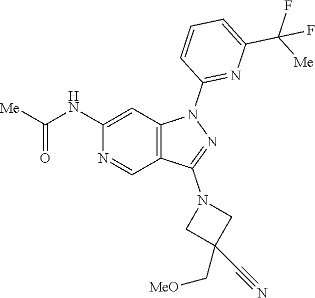 [SMILES: COCC1(C#N)CN(c2nn(-c3cccc(C(C)(F)F)n3)c3cc(NC(C)=O)ncc23)C1] | LCMS m/z = 442 [M + H]⁺ ¹H NMR (DMSO-d₆, 500 MHz) δ 10.60-10.80 (m, 1H), 9.30 (s, 1H), 8.79 (d, 1H, J = 1.1 Hz), 8.1-8.2 (m, 1H), 7.90 (d, 1H, J = 8.4 Hz), 7.52 (d, 1H, J = 7.3 Hz), 4.57 (d, 2H, J = 7.9 Hz), 4.36 (d, 2H, J = 8.1 Hz), 3.87 (s, 2H), 2.25 (t, 3H, J = 19.5 Hz), 2.15 (s, 3H). |
| | N-(3-(3-cyano-3- | |
| | (methoxymethyl)azetidin-1-yl)-1-(6-(1,1- | |
| | difluoroethyl)pyridin-2-yl)-1H- | |
| | pyrazolo[4,3-c]pyridin-6-yl)acetamide | |
| 574 | 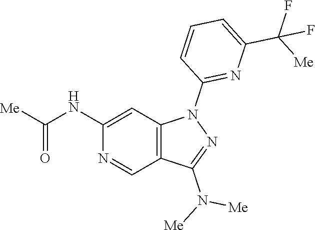 [SMILES: CC(=O)Nc1cc2c(cn1)c(N(C)C)nn2-c1cccc(C(C)(F)F)n1] | LCMS m/z = 361 [M + H]⁺ ¹H NMR (DMSO-d₆, 500 MHz) δ 10.67 (s, 1H), 9.33 (s, 1H), 9.01 (d, 1H, J = 0.6 Hz), 8.10-8.12 (m, 1H), 7.94 (d, 1H, J = 8.2 Hz), 7.48 (d, 1H, J = 7.2 Hz), 3.23 (s, 6H), 2.26 (t, 3H, J = 19.5 Hz), 2.15 (s, 3H). |
| | N-(1-(6-(1,1-difluoroethyl)pyridin-2-yl)- | |
| | 3-(dimethylamino)-1H-pyrazolo[4,3- | |
| | c]pyridin-6-yl)acetamide | |
| 575 | 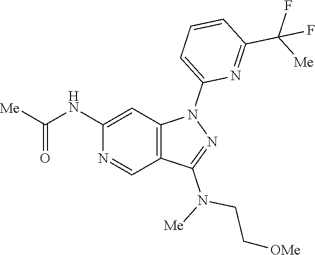 [SMILES: COCCN(C)c1nn(-c2cccc(C(C)(F)F)n2)c2cc(NC(C)=O)ncc12] | LCMS m/z = 405 [M + H]⁺ 1H NMR (DMSO-d₆, 500 MHz) δ 10.65 (s, 1H), 9.33 (s, 1H), 8.96 (s, 1H), 8.09 (t, 1H, J = 8.0 Hz), 7.92 (d, 1H, J = 8.4 Hz), 7.48 (d, 1H, J = 7.6 Hz), 3.80 (t, 1H, J = 5.3 Hz), 3.40-3.50 (m, 3H), 3.27 (d, 5H, J = 4.3 Hz), 2.25 (t, 3H, J = 19.4 Hz), 2.10-2.20 (m, 3H). |
| | N-(1-(6-(1,1-difluoroethyl)pyridin-2-y1)- | |
| | 3-((2-methoxyethyl)(methyl)amino)-1H- | |
| | pyrazolo[4,3-c]pyridin-6-yl)acetamide | |
| 576 | 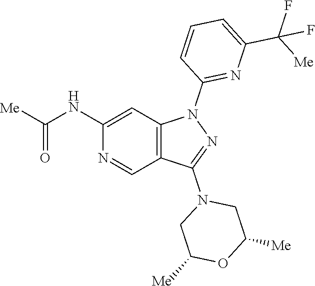 [SMILES: CC(=O)Nc1cc2c(cn1)c(N1C[C@@H](C)O[C@@H](C)C1)nn2-c1cccc(C(C)(F)F)n1] | LCMS m/z = 431 [M + H]⁺ 1H NMR (DMSO-d₆, 500 MHz) δ 10.70 (s, 1H), 9.35 (s, 1H), 9.12 (s, 1H), 8.11 (t, 1H, J = 7.9 Hz), 7.97 (d, 1H, J = 8.4 Hz), 7.51 (d, 1H, J = 7.5 Hz), 4.02 (br d, 2H, J = 12.2 Hz), 3.80-3.90 (m, 1H), 3.4-3.4 (m, 1H), 2.69 (br t, 2H, J = 11.4 Hz), 2.25 (br t, 3H, J = 19.5 Hz), 2.10-2.20 (m, 3H), 1.21 (br d, 6H, J = 6.1 Hz). |
| | N-(1-(6-(1,1-difluoroethyl)pyridin-2-yl)- | |
| | 3-((2R,6S)-2,6-dimethylmorpholino)-1H- | |
| | pyrazolo[4,3-c]pyridin-6-yl)acetamide | |
| 577 | 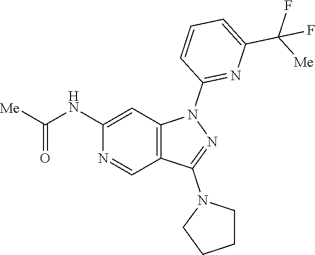 [SMILES: CC(=O)Nc1cc2c(cn1)c(N1CCCC1)nn2-c1cccc(C(C)(F)F)n1] | LCMS m/z = 387 [M + H]⁺ 1H NMR (DMSO-d₆, 500 MHz) δ 10.65 (s, 1H), 9.31 (s, 1H), 8.92 (d, 1H, J = 0.9 Hz), 8.00-8.10 (m, 1H), 7.92 (d, 1H, J = 8.4 Hz), 7.46 (dd, 1H, J = 0.6, 7.6 Hz), 3.60-3.80 (m, 4H), 2.26 (t, 3H, J = 19.5 Hz), 2.10- 2.20 (m, 3H), 2.00-2.02 (m, 4H). |
| | N-(1-(6-(1,1-difluoroethyl)pyridin-2-yl)- | |
| | 3-(pyrrolidin-1-yl)-1H-pyrazolo[4,3- | |
| | c]pyridin-6-yl)acetamide | |
| 578 | 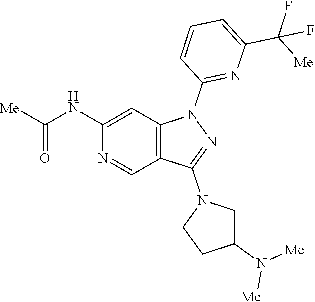 [SMILES: CC(=O)Nc1cc2c(cn1)c(N1CCC(N(C)C)C1)nn2-c1cccc(C(C)(F)F)n1] | LCMS m/z = 430 [M + H]⁺ 1H NMR (DMSO-d₆, 600 MHz) δ 10.63 (s, 1H), 9.33 (s, 1H), 9.00 (s, 1H), 8.11 (t, 1H, J = 8.0 Hz), 7.95 (d, 1H, J = 8.7 Hz), 7.50 (d, 1H, J = 7.3 Hz), 4.10 (q, 2H, J = 6.8 Hz), 3.98 (dt, 1H, J = 3.6, 9.1 Hz), 3.8- 3.9 (m, 1H), 3.74 (q, 1H, J = 8.0 Hz), 2.93 (br d, 6H, J = 9.4 Hz), 2.5- 2.6 (m, 1H), 2.25 (t, 4H, J = 19.3 Hz), 2.16 (s, 3H). |
| | N-(1-(6-(1,1-difluoroethyl)pyridin-2-yl)- | |
| | 3-(3-(dimethylamino)pyrrolidin-1-yl)-1H- | |
| | pyrazolo[4,3-c]pyridin-6-yl)acetamide | |
| 579 | 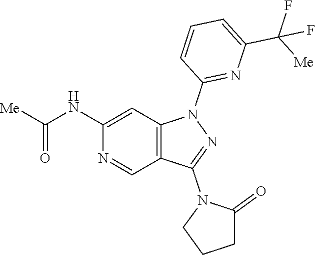 [SMILES: CC(=O)Nc1cc2c(cn1)c(N1CCCC1=O)nn2-c1cccc(C(C)(F)F)n1] | LCMS m/z = 401 [M + H]⁺ |
| | N-(1-(6-(1,1-difluoroethyl)pyridin-2-yl)- | |
| | 3-(2-oxopyrrolidin-1-yl)-1H-pyrazolo[4,3- | |
| | c]pyridin-6-yl)acetamide | |
| 580 | 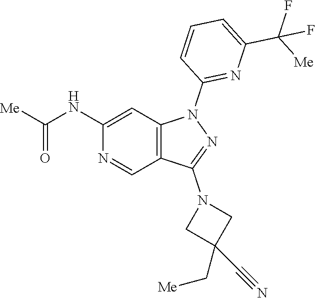 [SMILES: CCC1(C#N)CN(c2nn(-c3cccc(C(C)(F)F)n3)c3cc(NC(C)=O)ncc23)C1] | LCMS m/z = 426 [M + H]⁺ |
| | N-(3-(3-cyano-3-ethylazetidin-1-y1)-1-(6- | |
| | (1,1-difluoroethyl)pyridin-2-yl)-1H- | |
| | pyrazolo[4,3-c]pyridin-6-yl)acetamide | |
| 581 | 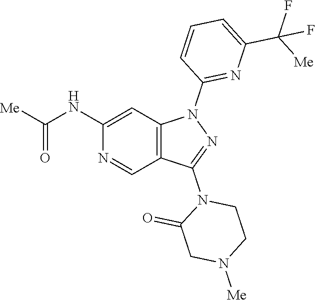 [SMILES: CC(=O)Nc1cc2c(cn1)c(N1CCN(C)CC1=O)nn2-c1cccc(C(C)(F)F)n1] | LCMS m/z = 430 [M + H]⁺ |
| | N-(1-(6-(1,1-difluoroethyl)pyridin-2-y1)- | |
| | 3-(4-methyl-2-oxopiperazin-1-yl)-1H- | |
| | pyrazolo[4,3-c]pyridin-6-yl)acetamide | |
| 582 | 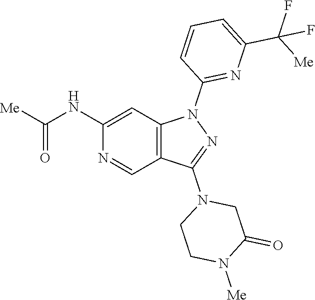 [SMILES: CC(=O)Nc1cc2c(cn1)c(N1CCN(C)C(=O)C1)nn2-c1cccc(C(C)(F)F)n1] | 1H NMR (DMSO-d6, 600 MHz) δ 10.7-10.7 (m, 1H), 9.34 (s, 1H), 9.07 (s, 1H), 8.11 (t, 1H, J = 8.0 Hz), 7.97 (d, 1H, J = 8.7 Hz), 7.52 (d, 1H, J = 7.3 Hz), 4.19 (s, 2H), 3.92 (t, 2H, J = 5.1 Hz), 3.55 (t, 2H, J = 5.4 Hz), 2.90-2.92 (m, 3H), 2.25 (t, 3H, J = 19.6 Hz), 2.15 (s, 3H). LCMS m/z = 430 [M + H]⁺ |
| | N-(1-(6-(1,1-difluoroethyl)pyridin-2-yl)- | |
| | 3-(3-methoxypiperidin-1-yl)-1H- | |
| | pyrazolo[4,3-c]pyridin-6-yl)acetamide | |
| 583 | 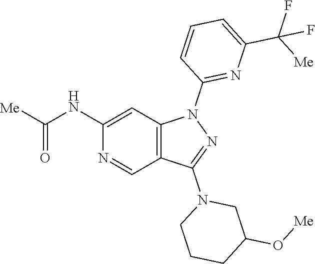 [SMILES: COC1CCCN(c2nn(-c3cccc(C(C)(F)F)n3)c3cc(NC(C)=O)ncc23)C1] | LCMS m/z = 431 [M + H]⁺ |
| | N-(1-(6-(1,1-difluoroethyl)pyridin-2-yl)- | |
| | 3-(3-methoxypiperidin-1-yl)-1H- | |
| | pyrazolo[4,3-c]pyridin-6-yl)acetamide | |
| 584 | 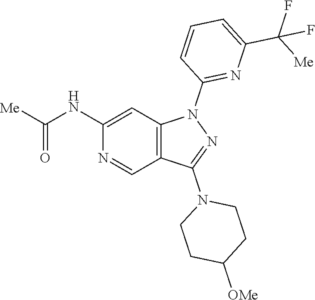 [SMILES: COC1CCN(c2nn(-c3cccc(C(C)(F)F)n3)c3cc(NC(C)=O)ncc23)CC1] | LCMS m/z = 431 [M + H]⁺ |
| | N-(1-(6-(1,1-difluoroethyl)pyridin-2-y1)- | |
| | 3-(4-methoxypiperidin-1-y1)-1H- | |
| | pyrazolo[4,3-c]pyridin-6-yl)acetamide | |
| 585 | 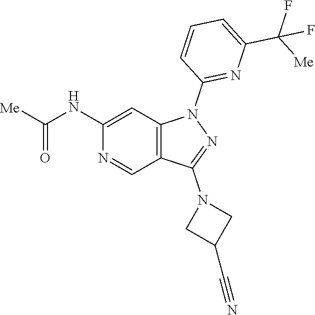 [SMILES: CC(=O)Nc1cc2c(cn1)c(N1CC(C#N)C1)nn2-c1cccc(C(C)(F)F)n1] | LCMS m/z = 399 [M + H]⁺ 1H NMR (DMSO-d₆, 500 MHz) 8 10.80 (s, 1H), 9.36 (d, 1H, J = 0.δ Hz), 8.88 (d, 1H, J = 5.8 Hz), 8.80 (d, 1H, J = 0.9 Hz), 7.79 (d, 1H, J = 5.8 Hz), 4.6-4.6 (m, 2H), 4.49 (dd, 2H, J = 5.6, 7.8 Hz), 4.06 (tt, 1H, J = 5.8, 8.7 Hz), 2.2-2.3 (m, 3H), 2.16 (s, 3H). |
| | N-(3-(3-cyanoazetidin-1-yl)-1-(4-(1,1- | |
| | difluoroethyl)pyrimidin-2-yl)-1H- | |
| | pyrazolo[4,3-c]pyridin-6-yl)acetamide | |
| 586 | 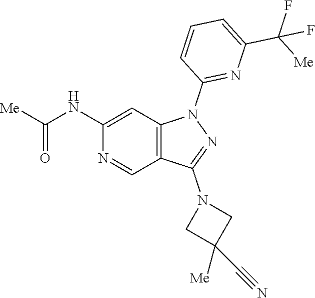 [SMILES: CC(=O)Nc1cc2c(cn1)c(N1CC(C)(C#N)C1)nn2-c1cccc(C(C)(F)F)n1] | LCMS m/z = 413 [M + H]⁺ 1H NMR (DMSO-d₆, 600 MHz) δ 10.71 (s, 1H), 9.26 (s, 1H), 9.0-9.1 (m, 1H), 8.76 (s, 1H), 7.6-7.6 (m, 1H), 4.61 (d, 2H, J = 8.0 Hz), 4.27 (d, 2H, J = 8.0 Hz), 2.2-2.3 (m, 3H), 2.15 (s, 3H), 1.72 (s, 3H). |
| | N-(3-(3-cyano-3-methylazetidin-1-yl)-1- | |
| | (4-(1,1-difluoroethyl)pyrimidin-2-y1)-1H- | |
| | pyrazolo[4,3-c]pyridin-6-yl)acetamide | |
| 587 | 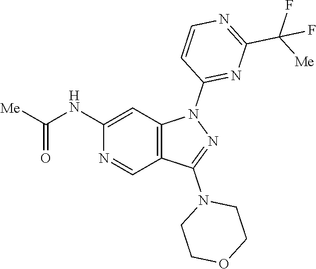 [SMILES: CC(=O)Nc1cc2c(cn1)c(N1CCOCC1)nn2-c1ccnc(C(C)(F)F)n1] | LCMS m/z = 404 [M + H]⁺ |
| | N-(1-(2-(1,1-difluoroethyl)pyrimidin-4- | |
| | yl)-3-morpholino-1H-pyrazolo[4,3- | |
| | c]pyridin-6-yl)acetamide | |
| 588 | 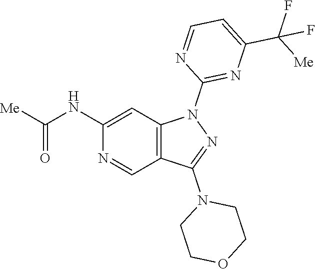 [SMILES: CC(=O)Nc1cc2c(cn1)c(N1CCOCC1)nn2-c1nccc(C(C)(F)F)n1] | LCMS m/z = 404 [M + H]⁺ |
| | N-(1-(4-(1,1-difluoroethyl)pyrimidin-2- | |
| | yl)-3-morpholino-1H-pyrazolo[4,3- | |
| | c]pyridin-6-yl)acetamide | |
| 589 | 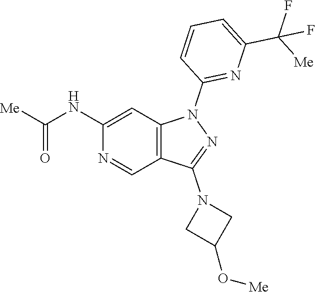 [SMILES: COC1CN(c2nn(-c3cccc(C(C)(F)F)n3)c3cc(NC(C)=O)ncc23)C1] | LCMS m/z = 403 [M + H]⁺ |
| | N-(1-(6-(1,1-difluoroethyl)pyridin-2-yl)- | |
| | 3-(3-methoxyazetidin-1-y1)-1H- | |
| | pyrazolo[4,3-c]pyridin-6-yl)acetamide | |
| 590 | 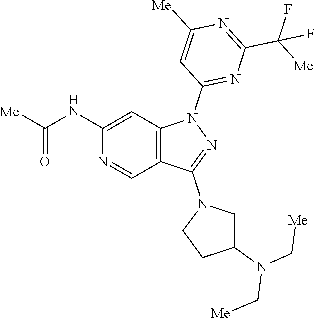 [SMILES: CCN(CC)C1CCN(c2nn(-c3cc(C)nc(C(C)(F)F)n3)c3cc(NC(C)=O)ncc23)C1] | LCMS m/z = 473 [M + H]⁺ |
| | N-(3-(3-(diethylamino)pyrrolidin-1-yl)-1- | |
| | (2-(1,1-difluoroethyl)-6-methylpyrimidin- | |
| | 4-y1)-1H-pyrazolo[4,3-c]pyridin-6- | |
| | yl)acetamide | |
| 591 | 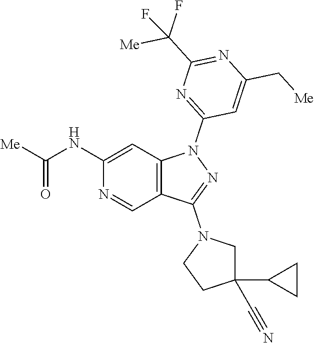 [SMILES: CCc1cc(-n2nc(N3CCC(C#N)(C4CC4)C3)c3cnc(NC(C)=O)cc32)nc(C(C)(F)F)n1] | LCMS m/z = 481 [M + H]⁺ |
| | N-(3-(3-cyano-3-cyclopropylpyrrolidin-1- | |
| | yl)-1-(2-(1,1-difluoroethyl)-6- | |
| | ethylpyrimidin-4-yl)-1H-pyrazolo[4,3- | |
| | c]pyridin-6-yl)acetamide | |
| 592 | 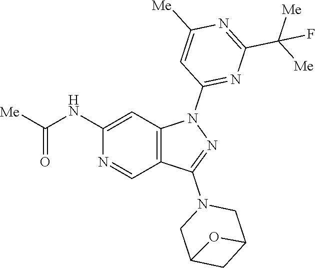 [SMILES: CC(=O)Nc1cc2c(cn1)c(N1CC3CC(C1)O3)nn2-c1cc(C)nc(C(C)(C)F)n1] | LCMS m/z = 426.3 [M + H]⁺ 1H NMR (MeOD-d₄ 400 MHz): δ ppm 9.43 (s, 1H), 8.96 (s, 1H), 7.61 (s, 1H), 4.80-4.84 (m, 2H), 3.96-4.13 (m, 4H), 3.33-3.38 (m, 1H), 2.57 (s, 3H), 2.23 (s, 3H), 2.14 (d, J = 9.0 Hz, 1H), 1.84-1.97 (m, 6H) |
| | N-(3-(6-oxa-3-azabicyclo[3.1.1]heptan-3- | |
| | yl)-1-(2-(2-fluoropropan-2-yl)-6- | |
| | methylpyrimidin-4-yl)-1H-pyrazolo[4,3- | |
| | c]pyridin-6-yl)acetamide | |
| 593 | 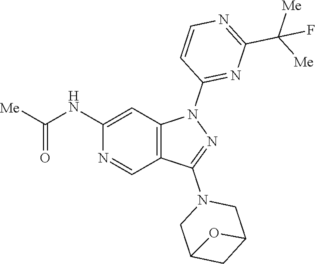 [SMILES: CC(=O)Nc1cc2c(cn1)c(N1CC3CC(C1)O3)nn2-c1ccnc(C(C)(C)F)n1] | LCMS m/z = 412.3 [M + H]⁺ 1H NMR (MeOD-d₄ 400 MHz): δ ppm 9.46 (s, 1H), 8.99 (d, J = 1.0 Hz, 1H), 8.66 (d, J = 5.5 Hz, 1H), 7.77 (d, J = 6.0 Hz, 1H), 4.81-4.84 (m, 2H), 3.97-4.12 (m, 4H), 3.33- 3.39 (m, 1H), 2.24 (s, 3H), 2.14 (d, J = 9.5 Hz, 1H), 1.85-1.98 (m, 6H) |
| | N-(3-(6-oxa-3-azabicyclo[3.1.1]heptan-3- | |
| | y1)-1-(2-(2-fluoropropan-2-yl)pyrimidin- | |
| | 4-yl)-1H-pyrazolo[4,3-c]pyridin-6- | |
| | yl)acetamide | |
| 594 | 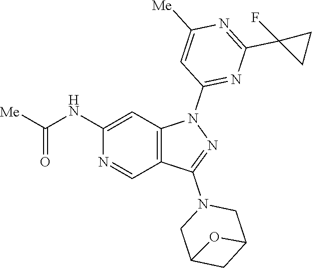 [SMILES: CC(=O)Nc1cc2c(cn1)c(N1CC3CC(C1)O3)nn2-c1cc(C)nc(C2(F)CC2)n1] | LCMS m/z = 424 [M + H]⁺ 1H NMR (400 MHz, MeOD-d₄) δ 9.10 (s, 1H), 8.80 (s, 1H), 7.90 (s, 1H), 4.59 (tt, 1H, J = 3.9, 8.1 Hz), 4.20- 4.30 (m, 2H), 3.91 (dd, 1H, J = 2.8, 13.5 Hz), 3.60-3.80 (m, 2H), 2.77 (s, 3H), 2.20-2.40 (m, 4H), 2.10- 2.20 (m, 1H), 1.90-2.00 (m, 4H). |
| | N-(3-(6-oxa-3-azabicyclo[3.1.1]heptan-3- | |
| | y1)-1-(2-(1-fluorocyclopropyl)-6- | |
| | methylpyrimidin-4-yl)-1H-pyrazolo[4,3- | |
| | c]pyridin-6-yl)acetamide | |
| 595 | 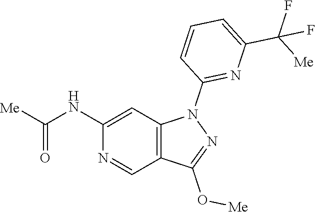 [SMILES: COc1nn(-c2cccc(C(C)(F)F)n2)c2cc(NC(C)=O)ncc12] | LCMS m/z = 348 [M + H]⁺ 1H NMR (400 MHz, CDCl₃) δ 9.42 (s, 1H), 8.66 (d, J = 1.00 Hz, 1H), 8.50 (br s, 1H), 7.88-7.97 (m, 2H), 7.51 (dd, J = 2.01, 6.53 Hz, 1H), 4.21 (s, 3H), 2.27-2.38 (m, 6H) |
| | N-(1-(6-(1,1-difluoroethyl)pyridin-2-yl)- | |
| | 3-methoxy-1H-pyrazolo[4,3-c]pyridin-6- | |
| | yl)acetamide | |
| 596 | 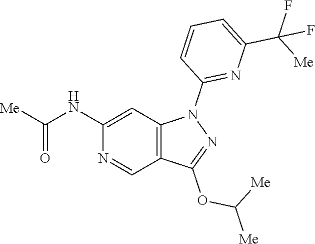 [SMILES: CC(=O)Nc1cc2c(cn1)c(OC(C)C)nn2-c1cccc(C(C)(F)F)n1] | LCMS m/z = 376 [M + H]⁺ 1H NMR (400 MHz, MeOD-d₄) δ 9.03 (s, 1H), 8.75 (s, 1H), 8.03-8.11 (m, 1H), 7.94-8.01 (m, 1H), 7.55 (d, J = 7.53 Hz, 1H), 5.33 (spt, J = 6.11 Hz, 1H), 2.15-2.30 (m, 6H), 1.54 (d, J = 6.27 Hz, 6H) |
| | N-(1-(6-(1,1-difluoroethyl)pyridin-2-yl)- | |
| | 3-isopropoxy-1H-pyrazolo[4,3-c]pyridin- | |
| | 6-yl)acetamide | |
| 597 | 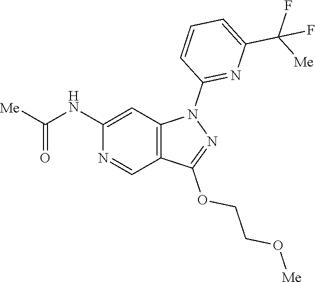 [SMILES: COCCOc1nn(-c2cccc(C(C)(F)F)n2)c2cc(NC(C)=O)ncc12] | LCMS m/z = 392 [M + H]⁺ 1H NMR (500 MHz, DMSO-d₆) δ 10.76 (s, 1H), 9.32 (s, 1H), 8.81 (d, J = 1.22 Hz, 1H), 8.11-8.19 (m, 1H), 7.94 (d, J = 8.55 Hz, 1H), 7.56 (d, J = 6.71 Hz, 1H), 4.63 (td, J = 2.29, 3.97 Hz, 2H), 3.81 (td, J = 2.29, 3.97 Hz, 2H), 3.31-3.32 (m, 3H), 2.20-2.31 (m, 3H), 2.09-2.20 (m, 3H). |
| | N-(1-(6-(1,1-difluoroethyl)pyridin-2-yl)- | |
| | 3-(2-methoxyethoxy)-1H-pyrazolo[4,3- | |
| | c]pyridin-6-yl)acetamide | |
| 598 | 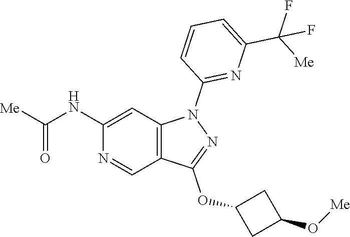 [SMILES: CO[C@H]1C[C@H](Oc2nn(-c3cccc(C(C)(F)F)n3)c3cc(NC(C)=O)ncc23)C1] | LCMS m/z = 418 [M + H]⁺ 1H NMR (400 MHz, CDCl₃) δ 10.14 (br s, 1H), 9.62 (s, 1H), 8.64 (s, 1H), 7.91-8.00 (m, 2H), 7.57 (d, J = 7.28 Hz, 1H), 5.43-5.51 (m, 1H), 4.18-4.26 (m, 1H), 3.31-3.35 (m, 3H), 2.53-2.68 (m, 4H), 2.24-2.35 (m, 6H) |
| | N-(1-(6-(1,1-difluoroethyl)pyridin-2-yl)- | |
| | 3-((1r,3r)-3-methoxycyclobutoxy)-1H- | |
| | pyrazolo[4,3-c]pyridin-6-yl)acetamide | |
| 599 | 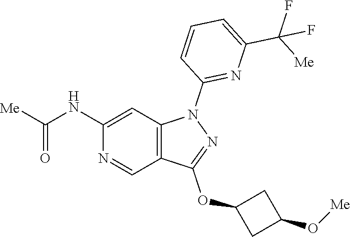 [SMILES: CO[C@H]1C[C@@H](Oc2nn(-c3cccc(C(C)(F)F)n3)c3cc(NC(C)=O)ncc23)C1] | LCMS m/z = 418 [M + H]⁺ 1H NMR (400 MHz, CDCl₃) δ 9.54 (s, 2H), 8.65 (s, 1H), 7.88-7.98 (m, 2H), 7.52-7.59 (m, 1H), 4.95 (quin, J = 7.09 Hz, 1H), 3.67-3.80 (m, 1H), 3.29-3.34 (m, 3H), 2.99-3.10 (m, 2H), 2.22-2.37 (m, 8H) |
| | N-(1-(6-(1,1-difluoroethyl)pyridin-2-yl)- | |
| | 3-((1s,3s)-3-methoxycyclobutoxy)-1H- | |
| | pyrazolo[4,3-c]pyridin-6-yl)acetamide | |
| 600 | 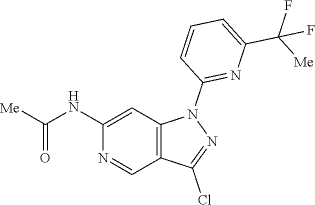 [SMILES: CC(=O)Nc1cc2c(cn1)c(Cl)nn2-c1cccc(C(C)(F)F)n1] | LCMS m/z = 352 [M + H]⁺ |
| | N-(3-chloro-1-(6-(1,1- | |
| | difluoroethyl)pyridin-2-y1)-1H- | |
| | pyrazolo[4,3-c]pyridin-6-yl)acetamide | |
| 601 | 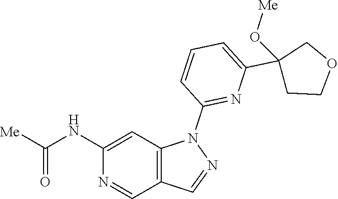 [SMILES: COC1(c2cccc(-n3ncc4cnc(NC(C)=O)cc43)n2)CCOC1] | LCMS m/z = 354 [M + H]⁺ 1H NMR (400 MHz, CDCl₃) δ 11.77 (br s, 1H), 9.85 (s, 1H), 8.82 (s, 1H), 8.56 (d, J = 0.75 Hz, 1H), 7.97- 8.08 (m, 2H), 7.62 (dd, J = 1.88, 6.65 Hz, 1H), 4.21-4.36 (m, 3H), 4.09-4.15 (m, 1H), 3.31 (s, 3H), 2.89 (td, J = 8.72, 13.43 Hz, 1H), 2.57-2.66 (m, 1H), 2.41 (s, 3H) |
| | N-(1-(6-(3-methoxytetrahydrofuran-3- | |
| | yl)pyridin-2-yl)-1H-pyrazolo[4,3- | |
| | c]pyridin-6-yl)acetamide | |
| 602 | 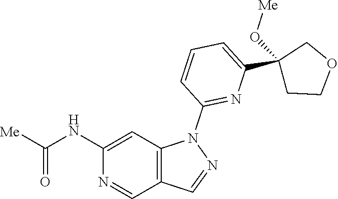 [SMILES: CO[C@@]1(c2cccc(-n3ncc4cnc(NC(C)=O)cc43)n2)CCOC1] | LCMS m/z = 354 [M + H]⁺ 1H NMR (400 MHz, CDCl₃) δ 11.77 (br s, 1H), 9.85 (s, 1H), 8.83 (s, 1H), 8.56 (s, 1H), 7.96-8.09 (m, 2H), 7.58-7.66 (m, 1H), 4.20-4.35 (m, 3H), 4.11 (d, J = 9.54 Hz, 1H), 3.31 (s, 3H), 2.89 (td, J = 8.60, 13.43 Hz, 1H), 2.56-2.67 (m, 1H), 2.41 (s, 3H). |
| | or | |
| | 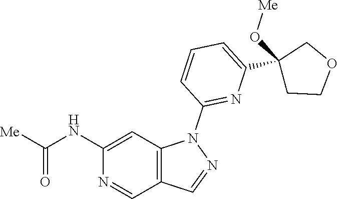 [SMILES: CO[C@]1(c2cccc(-n3ncc4cnc(NC(C)=O)cc43)n2)CCOC1] | |
| | (R)-N-(1-(6-(3-methoxytetrahydrofuran-3- | |
| | yl)pyridin-2-yl)-1H-pyrazolo[4,3- | |
| | c]pyridin-6-yl)acetamide or (S)-N-(1-(6- | |
| | (3-methoxytetrahydrofuran-3-yl)pyridin- | |
| | 2-y1)-1H-pyrazolo[4,3-c]pyridin-6- | |
| | yl)acetamide | |
| 603 | 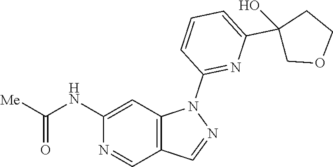 [SMILES: CC(=O)Nc1cc2c(cn1)cnn2-c1cccc(C2(O)CCOC2)n1] | LCMS m/z = 340 [M + H]⁺ |
| | N-(1-(6-(3-hydroxytetrahydrofuran-3- | |
| | yl)pyridin-2-yl)-1H-pyrazolo[4,3- | |
| | c]pyridin-6-yl)acetamide | |
| 604 | 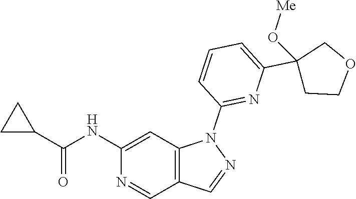 [SMILES: COC1(c2cccc(-n3ncc4cnc(NC(=O)C5CC5)cc43)n2)CCOC1] | LCMS m/z = 380 [M + H]⁺ |
| | N-(1-(6-(3-methoxytetrahydrofuran-3- | |
| | yl)pyridin-2-yl)-1H-pyrazolo[4,3- | |
| | c]pyridin-6-yl)cyclopropanecarboxamide | |
| 605 | 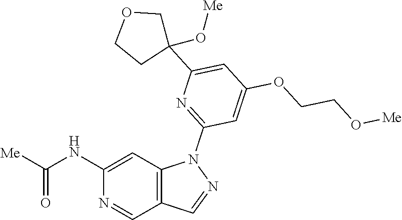 [SMILES: COCCOc1cc(-n2ncc3cnc(NC(C)=O)cc32)nc(C2(OC)CCOC2)c1] | LCMS m/z = 428 [M + H]⁺ 1H NMR (400 MHz, MeOD-d₄) δ 9.31 (s, 1H), 8.86 (d, J = 1.00 Hz, 1H), 8.36 (d, J = 0.75 Hz, 1H), 7.55 (d, J = 2.26 Hz, 1H), 7.06 (d, J = 2.26 Hz, 1H), 4.32-4.36 (m, 2H), 4.28 (dd, J = 1.25, 9.79 Hz, 1H), 4.19- 4.26 (m, 1H), 4.11-4.18 (m, 1H), 4.08 (d, J = 9.54 Hz, 1H), 3.80-3.84 (m, 2H), 3.45 (s, 3H), 3.29 (s, 3H), 2.90 (td, J = 8.78, 13.30 Hz, 1H), 2.53-2.62 (m, 1H), 2.23 (s, 3H). |
| | N-(1-(4-(2-methoxyethoxy)-6-(3- | |
| | methoxytetrahydrofuran-3-yl)pyridin-2- | |
| | yl)-1H-pyrazolo[4,3-c]pyridin-6- | |
| | yl)acetamide | |
| 606 | 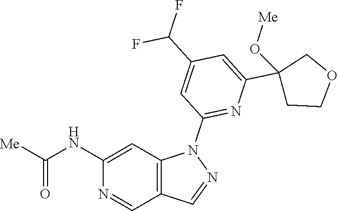 [SMILES: COC1(c2cc(C(F)F)cc(-n3ncc4cnc(NC(C)=O)cc43)n2)CCOC1] | LCMS m/z = 404 [M + H]⁺ |
| | N-(1-(4-(difluoromethyl)-6-(3- | |
| | methoxytetrahydrofuran-3-yl)pyridin-2- | |
| | yl)-1H-pyrazolo[4,3-c]pyridin-6- | |
| | yl)acetamide | |
| 607 | 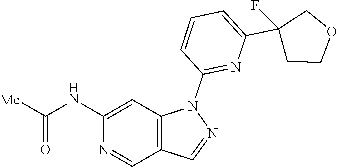 [SMILES: CC(=O)Nc1cc2c(cn1)cnn2-c1cccc(C2(F)CCOC2)n1] | LCMS m/z = 342 [M + H]⁺ 1H NMR (400 MHz, CDCl₃) δ 12.26 (br s, 1H), 9.86 (s, 1H), 8.98 (s, 1H), 8.56 (s, 1H), 7.98-8.15 (m, 2H), 7.68-7.81 (m, 1H), 4.53 (dt, J = 3.39, 8.60 Hz, 1H), 4.28-4.43 (m, 3H), 2.97-3.19 (m, 1H), 2.52- 2.68 (m, 1H), 2.38 (s, 3H) |
| | N-(1-(6-(3-fluorotetrahydrofuran-3- | |
| | yl)pyridin-2-yl)-1H-pyrazolo[4,3- | |
| | c]pyridin-6-yl)acetamide | |
| 608 | 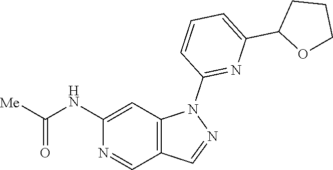 [SMILES: CC(=O)Nc1cc2c(cn1)cnn2-c1cccc(C2CCCO2)n1] | LCMS m/z = 324 [M + H]⁺ |
| | N-(1-(6-(tetrahydrofuran-2-yl)pyridin-2- | |
| | yl)-1H-pyrazolo[4,3-c]pyridin-6- | |
| | yl)acetamide | |
| 609 | 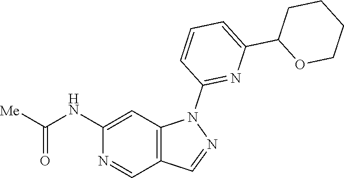 [SMILES: CC(=O)Nc1cc2c(cn1)cnn2-c1cccc(C2CCCCO2)n1] | LCMS m/z = 338 [M + H]⁺ |
| | N-(1-(6-(tetrahydro-2H-pyran-2- | |
| | yl)pyridin-2-yl)-1H-pyrazolo[4,3- | |
| | c]pyridin-6-yl)acetamide | |
| 610 | 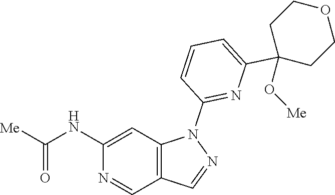 [SMILES: COC1(c2cccc(-n3ncc4cnc(NC(C)=O)cc43)n2)CCOCC1] | LCMS m/z = 368 [M + H]+ |
| | N-(1-(6-(4-methoxytetrahydro-2H-pyran- | |
| | 4-yl)pyridin-2-y1)-1H-pyrazolo[4,3- | |
| | c]pyridin-6-yl)acetamide | |
| 611 | 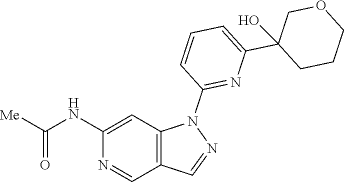 [SMILES: CC(=O)Nc1cc2c(cn1)cnn2-c1cccc(C2(O)CCCOC2)n1] | LCMS m/z = 353 [M + H]⁺ |
| | N-(1-(6-(3-hydroxytetrahydro-2H-pyran- | |
| | 3-yl)pyridin-2-yl)-1H-pyrazolo[4,3- | |
| | c]pyridin-6-yl)acetamide | |
| 612 | 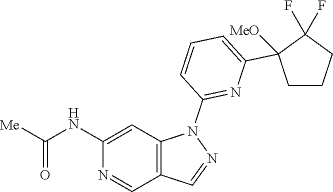 [SMILES: COC1(c2cccc(-n3ncc4cnc(NC(C)=O)cc43)n2)CCCC1(F)F] | LCMS m/z = 388 [M + H]⁺ 1H NMR (400 MHz, CDCl₃) δ 9.38 (s, 1H), 8.79 (d, J = 0.75 Hz, 1H), 8.19- 8.31 (m, 2H), 7.99-8.06 (m, 1H), 7.91-7.97 (m, 1H), 7.65 (d, J = 7.53 Hz, 1H), 3.23 (s, 3H), 3.08-3.20 (m, 1H), 2.65-2.75 (m, 1H), 2.37- 2.58 (m, 1H), 2.20-2.36 (m, 4H), 1.97-2.15 (m, 2H). |
| | N-(1-(6-(2,2-difluoro-1- | |
| | methoxycyclopentyl)pyridin-2-yl)-1H- | |
| | pyrazolo[4,3-c]pyridin-6-yl)acetamide | |
| 613 | 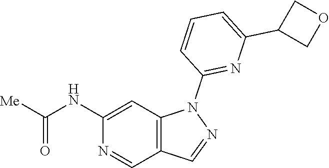 [SMILES: CC(=O)Nc1cc2c(cn1)cnn2-c1cccc(C2COC2)n1] | LCMS m/z = 310 [M + H]⁺ 1H NMR (400 MHz, CDCl₃) δ 9.63 (s, 1H), 8.82-8.93 (m, 1H), 8.79 (d, J = 1.00 Hz, 1H), 8.30 (s, 1H), 7.91- 7.96 (m, 1H), 7.83-7.90 (m, 1H), 7.21 (d, J = 7.28 Hz, 1H), 5.13-5.23 (m, 4H), 4.51-4.62 (m, 1H), 2.31 (s, 3H). |
| | N-(1-(6-(oxetan-3-yl)pyridin-2-yl)-1H- | |
| | pyrazolo[4,3-c]pyridin-6-yl)acetamide | |
| 614 | 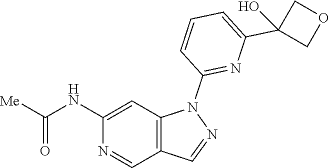 [SMILES: CC(=O)Nc1cc2c(cn1)cnn2-c1cccc(C2(O)COC2)n1] | LCMS m/z = 325 [M + H]⁺ |
| | N-(1-(6-(3-hydroxyoxetan-3-yl)pyridin-2- | |
| | yl)-1H-pyrazolo[4,3-c]pyridin-6- | |
| | yl)acetamide | |
| 615 | 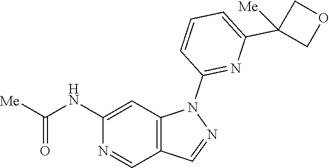 [SMILES: CC(=O)Nc1cc2c(cn1)cnn2-c1cccc(C2(C)COC2)n1] | LCMS m/z = 323 [M + H]⁺ |
| | N-(1-(6-(3-methyloxetan-3-y1)pyridin-2- | |
| | yl)-1H-pyrazolo[4,3-c]pyridin-6- | |
| | yl)acetamide | |
| 616 | 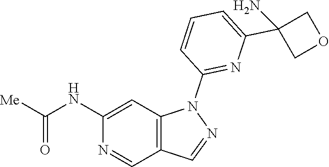 [SMILES: CC(=O)Nc1cc2c(cn1)cnn2-c1cccc(C2(N)COC2)n1] | LCMS m/z = 325 [M + H]⁺ 1H NMR (500 MHz, DMSO-d₆) δ 10.90 (s, 1H), 9.35 (s, 1H), 8.99 (d, J = 1.07 Hz, 1H), 8.61 (d, J = 0.92 Hz, 1H), 8.22-8.31 (m, 1H), 8.08 (d, J = 8.24 Hz, 1H), 7.89 (d, J = 7.63 Hz, 1H), 5.03 (d, J = 7.63 Hz, 2H), 4.92 (d, J = 7.78 Hz, 2H), 2.21 (s, 3H). |
| | N-(1-(6-(3-aminooxetan-3-yl)pyridin-2- | |
| | yl)-1H-pyrazolo[4,3-c]pyridin-6- | |
| | yl)acetamide | |
| 617 | 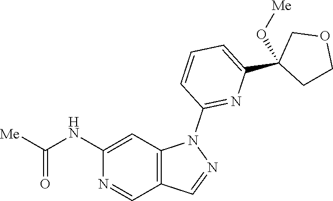 [SMILES: CO[C@@]1(c2cccc(-n3ncc4cnc(NC(C)=O)cc43)n2)CCOC1] | LCMS m/z = 354 [M + H]⁺ 1H NMR (400 MHz, CDCl₃) δ 11.77 (br s, 1H), 9.85 (s, 1H), 8.82 (s, 1H), 8.56 (s, 1H), 7.97-8.08 (m, 2H), 7.63 (dd, J = 2.01, 6.78 Hz, 1H), 4.19-4.35 (m, 3H), 4.11 (d, J = 9.79 Hz, 1H), 3.31 (s, 3H), 2.85- 2.96 (m, 1H), 2.56-2.68 (m, 1H), 2.41 (s, 3H). |
| | Or | |
| | 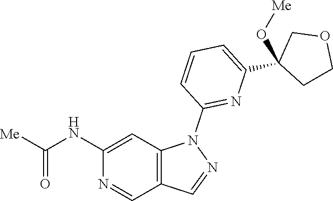 [SMILES: CO[C@]1(c2cccc(-n3ncc4cnc(NC(C)=O)cc43)n2)CCOC1] | |
| | (R)-N-(1-(6-(3-methoxytetrahydrofuran-3- | |
| | yl)pyridin-2-yl)-1H-pyrazolo[4,3- | |
| | c]pyridin-6-yl)acetamide or (S)-N-(1-(6- | |
| | (3-methoxytetrahydrofuran-3-yl)pyridin- | |
| | 2-y1)-1H-pyrazolo[4,3-c]pyridin-6- | |
| | yl)acetamide | |
| 618 | 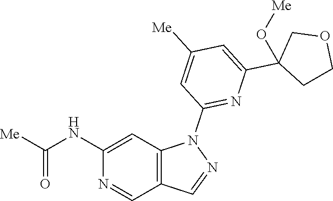 [SMILES: COC1(c2cc(C)cc(-n3ncc4cnc(NC(C)=O)cc43)n2)CCOC1] | LCMS m/z = 368 [M + H]⁺ 1H NMR (500 MHz, DMSO-d₆) δ 10.67 (s, 1H), 9.28 (s, 1H), 8.95 (d, J = 1.22 Hz, 1H), 8.53 (d, J = 0.76 Hz, 1H), 7.79 (s, 1H), 7.32 (s, 1H), 4.26 (dd, J = 1.14, 9.69 Hz, 1H), 4.06 (dt, J = 3.74, 8.35 Hz, 1H), 3.97-4.03 (m, 1H), 3.91 (d, J = 9.61 Hz, 1H), 3.14 (s, 3H), 2.73 (td, J = 8.77, 13.28 Hz, 1H), 2.54 (dtd, J = 1.22, 3.26, 6.75 Hz, 1H), 2.48 (s, 3H), 2.15 (s, 3H). |
| | N-(1-(6-(3-methoxytetrahydrofuran-3-yl)- | |
| | 4-methylpyridin-2-yl)-1H-pyrazolo[4,3- | |
| | c]pyridin-6-yl)acetamide | |
| 619 | 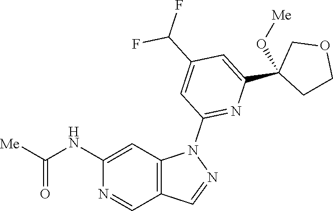 [SMILES: CO[C@@]1(c2cc(C(F)F)cc(-n3ncc4cnc(NC(C)=O)cc43)n2)CCOC1] | LCMS m/z = 404 [M + H]⁺ 1H NMR (400 MHz, MeOD-d₄) δ 9.33 (s, 1H), 8.88 (d, J = 1.00 Hz, 1H), 8.41 (d, J = 0.75 Hz, 1H), 8.15 (s, 1H), 7.62 (s, 1H), 6.78-7.18 (m, 1H), 4.34 (d, J = 9.79 Hz, 1H), 4.26 (dt, J = 3.89, 8.34 Hz, 1H), 4.14- 4.22 (m, 1H), 4.11 (d, J = 9.54 Hz, 1H), 3.30 (br s, 3H), 2.93 (td, J = 8.75, 13.36 Hz, 1H), 2.59-2.71 (m, 1H), 2.23 (s, 3H) |
| | or | |
| | 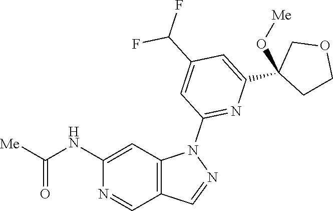 [SMILES: CO[C@]1(c2cc(C(F)F)cc(-n3ncc4cnc(NC(C)=O)cc43)n2)CCOC1] | |
| | (R)-N-(1-(4-(difluoromethyl)-6-(3- | |
| | methoxytetrahydrofuran-3-y1)pyridin-2- | |
| | yl)-1H-pyrazolo[4,3-c]pyridin-6- | |
| | yl)acetamide or (S)-N-(1-(4- | |
| | (difluoromethyl)-6-(3- | |
| | methoxytetrahydrofuran-3-y1)pyridin-2- | |
| | yl)-1H-pyrazolo[4,3-c]pyridin-6- | |
| | yl)acetamide | |
| 620 | 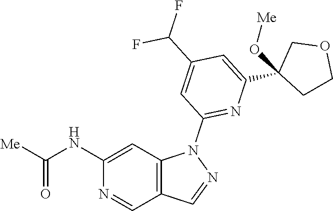 [SMILES: CO[C@]1(c2cc(C(F)F)cc(-n3ncc4cnc(NC(C)=O)cc43)n2)CCOC1] | LCMS m/z = 404 [M + H]⁺ 1H NMR (400 MHz, MeOD-d₄) δ 9.34 (s, 1H), 8.88 (s, 1H), 8.41 (s, 1H), 8.15 (s, 1H), 7.62 (s, 1H), 6.80- 7.17 (m, 1H), 4.34 (d, J = 9.79 Hz, 1H), 4.27 (dt, J = 4.02, 8.41 Hz, 1H), 4.14-4.22 (m, 1H), 4.11 (d, J = 9.79 Hz, 1H), 3.30-3.30 (m, 3H), 2.94 (td, J = 8.69, 13.49 Hz, 1H), 2.58-2.71 (m, 1H), 2.23 (s, 3H). |
| | or | |
| | 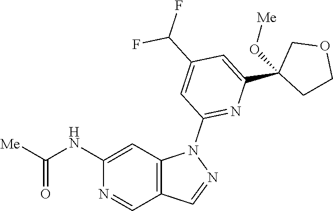 [SMILES: CO[C@@]1(c2cc(C(F)F)cc(-n3ncc4cnc(NC(C)=O)cc43)n2)CCOC1] | |
| | (S)-N-(1-(4-(difluoromethyl)-6-(3- | |
| | methoxytetrahydrofuran-3-yl)pyridin-2- | |
| | yl)-1H-pyrazolo[4,3-c]pyridin-6- | |
| | yl)acetamide or (R)-N-(1-(4- | |
| | (difluoromethyl)-6-(3- | |
| | methoxytetrahydrofuran-3-yl)pyridin-2- | |
| | yl)-1H-pyrazolo[4,3-c]pyridin-6- | |
| | yl)acetamide | |
| 621 | 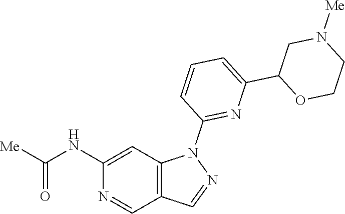 [SMILES: CC(=O)Nc1cc2c(cn1)cnn2-c1cccc(C2CN(C)CCO2)n1] | LCMS m/z = 353 [M + H]⁺ 1H NMR (500 MHz, DMSO-d₆) δ 10.68 (s, 1H), 9.34 (s, 1H), 8.95 (d, J = 1.07 Hz, 1H), 8.54 (d, J = 0.76 Hz, 1H), 8.03 (t, J = 7.93 Hz, 1H), 7.89 (d, J = 8.09 Hz, 1H), 7.41 (d, J = 7.63 Hz, 1H), 4.64 (dd, J = 2.44, 10.22 Hz, 1H), 4.02 (td, J = 1.70, 9.57 Hz, 1H), 3.77 (dt, J = 2.44, 11.29 Hz, 1H), 3.63 (br d, J = 11.14 Hz, 1H), 2.71 (br d, J = 11.60 Hz, 1H), 2.35 (s, 3H), 2.10-2.16 (m, 4H), 2.02 (t, J = 10.83 Hz, 1H). |
| | N-(1-(6-(4-methylmorpholin-2-yl)pyridin- | |
| | 2-y1)-1H-pyrazolo[4,3-c]pyridin-6- | |
| | yl)acetamide | |
| 622 | 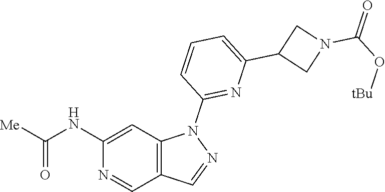 [SMILES: CC(=O)Nc1cc2c(cn1)cnn2-c1cccc(C2CN(C(=O)OC(C)(C)C)C2)n1] | LCMS m/z = 409 [M + H]⁺ 1H NMR (400 MHz, MeOD-d₄) δ 9.35 (s, 1H), 8.87 (d, J = 1.00 Hz, 1H), 8.37 (d, J = 0.75 Hz, 1H), 7.88-7.99 (m, 2H), 7.23-7.31 (m, 1H), 4.40- 4.50 (m, 2H), 4.31 (br s, 2H), 4.08- 4.20 (m, 1H), 2.23 (s, 3H), 1.46 (s, 9H). |
| | tert-butyl 3-(6-(6-acetamido-1H- | |
| | pyrazolo[4,3-c]pyridin-1-yl)pyridin-2- | |
| | yl)azetidine-1-carboxylate | |
| 623 | 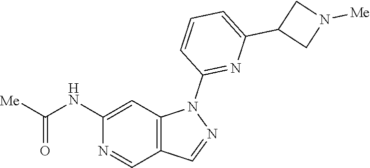 [SMILES: CC(=O)Nc1cc2c(cn1)cnn2-c1cccc(C2CN(C)C2)n1] | LCMS m/z = 323 [M + H]⁺ |
| | N-(1-(6-(1-methylazetidin-3-y1)pyridin-2- | |
| | yl)-1H-pyrazolo[4,3-c]pyridin-6- | |
| | yl)acetamide | |
| 624 | 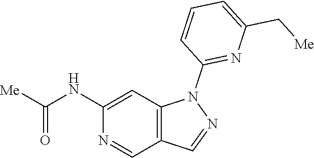 [SMILES: CCc1cccc(-n2ncc3cnc(NC(C)=O)cc32)n1] | LCMS m/z = 282 [M + H]⁺ 1H NMR (500 MHz, DMSO-d₆) δ 10.66 (s, 1H), 9.46 (s, 1H), 8.94 (d, J = 1.07 Hz, 1H), 8.52 (d, J = 0.92 Hz, 1H), 7.88-7.96 (m, 1H), 7.78 (d, J = 8.09 Hz, 1H), 7.22 (d, J = 7.17 Hz, 1H), 2.88 (q, J = 7.58 Hz, 2H), 2.15 (s, 3H), 1.42 (t, J = 7.63 Hz, 3H). |
| | N-(1-(6-ethylpyridin-2-y1)-1H- | |
| | pyrazolo[4,3-c]pyridin-6-yl)acetamide | |
| 625 | 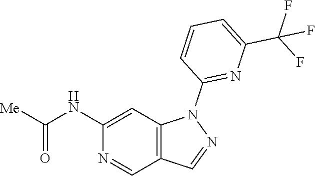 [SMILES: CC(=O)Nc1cc2c(cn1)cnn2-c1cccc(C(F)(F)F)n1] | LCMS m/z = 322 [M + H]⁺ 1H NMR (500 MHz, DMSO-d₆) δ 10.69 (s, 1H), 9.40 (s, 1H), 8.97 (d, J = 1.07 Hz, 1H), 8.61 (d, J = 0.92 Hz, 1H), 8.23-8.35 (m, 2H), 7.85 (dd, J = 0.92, 7.17 Hz, 1H), 2.15 (s, 3H). |
| | N-(1-(6-(trifluoromethyl)pyridin-2-yl)- | |
| | 1H-pyrazolo[4,3-c]pyridin-6-yl)acetamide | |
| 626 | 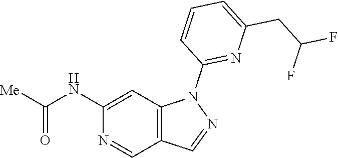 [SMILES: CC(=O)Nc1cc2c(cn1)cnn2-c1cccc(CC(F)F)n1] | LCMS m/z = 318 [M + H]⁺ 1H NMR (400 MHz, DMSO-d₆) δ 10.73 (s, 1H), 9.42 (s, 1H), 8.96 (d, J = 1.00 Hz, 1H), 8.55 (d, J = 0.75 Hz, 1H), 7.97-8.07 (m, 1H), 7.90 (d, J = 8.28 Hz, 1H), 7.35 (d, J = 7.28 Hz, 1H), 6.69-7.08 (m, 1H), 3.53 (dt, J = 5.02, 16.94 Hz, 2H), 2.17 (s, 3H) |
| | N-(1-(6-(2,2-difluoroethyl)pyridin-2-yl)- | |
| | 1H-pyrazolo[4,3-c]pyridin-6-yl)acetamide | |
| 627 | 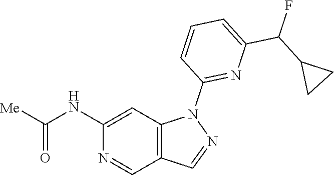 [SMILES: CC(=O)Nc1cc2c(cn1)cnn2-c1cccc(C(F)C2CC2)n1] | LCMS m/z = 326 [M + H]⁺ 1H NMR (400 MHz, MeOD-d₄) δ 9.42 (s, 1H), 8.86 (d, J = 1.00 Hz, 1H), 8.36 (s, 1H), 7.96-8.04 (m, 2H), 7.42 (dd, J = 2.76, 5.77 Hz, 1H), 4.94-5.12 (m, 1H), 2.23 (s, 3H), 1.55-1.68 (m, 1H), 0.64-0.82 (m, 4H) |
| | (cyclopropylfluoromethyl)pyridin-2-y1)- | |
| | 1H-pyrazolo[4,3-c]pyridin-6-yl)acetamide | |
| 628 | 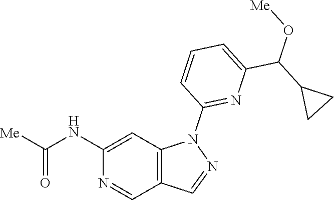 [SMILES: COC(c1cccc(-n2ncc3cnc(NC(C)=O)cc32)n1)C1CC1] | LCMS m/z = 338 [M + H]⁺ 1H NMR (400 MHz, MeOD-d₄) δ 9.46 (s, 1H), 8.86 (d, J = 1.26 Hz, 1H), 8.36 (d, J = 0.75 Hz, 1H), 7.90-8.03 (m, 2H), 7.38 (dd, J = 1.25, 7.03 Hz, 1H), 3.89 (d, J = 8.03 Hz, 1H), 3.40 (s, 3H), 2.24 (s, 3H), 1.27-1.39 (m, 1H), 0.58-0.71 (m, 3H), 0.44-0.52 (m, 1H). |
| | (cyclopropyl(methoxy)methyl)pyridin-2- | |
| | y1)-1H-pyrazolo[4,3-c]pyridin-6- | |
| | yl)acetamide | |
| 629 | 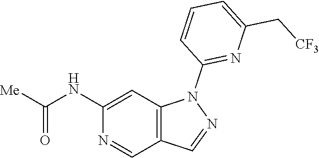 [SMILES: CC(=O)Nc1cc2c(cn1)cnn2-c1cccc(CC(F)(F)F)n1] | LCMS m/z = 336 [M + H]⁺ 1H NMR (500 MHz, DMSO-d₆) $10.65 (s, 1H), 9.41 (s, 1H), 8.95 (d, J = 1.07 Hz, 1H), 8.55 (d, J = 0.61 Hz, 1H), 8.04-8.12 (m, 1H), 7.97 (d, J = 8.24 Hz, 1H), 7.44 (d, J = 7.48 Hz, 1H), 3.90 (q, J = 11.29 Hz, 2H), 2.15 (s, 3H). |
| | N-(1-(6-(2,2,2-trifluoroethyl)pyridin-2- | |
| | yl)-1H-pyrazolo[4,3-c]pyridin-6- | |
| | yl)acetamide | |
| 630 | 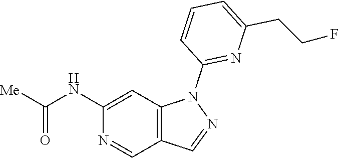 [SMILES: CC(=O)Nc1cc2c(cn1)cnn2-c1cccc(CCF)n1] | LCMS m/z = 300 [M + H]⁺ 1H NMR (500 MHz, DMSO-d₆) δ 10.70 (s, 1H), 9.46 (s, 1H), 8.95 (d, J = 0.92 Hz, 1H), 8.54 (d, J = 0.76 Hz, 1H), 7.94-7.99 (m, 1H), 7.85 (d, J = 8.09 Hz, 1H), 7.30 (d, J = 7.48 Hz, 1H), 5.15 (t, J = 6.18 Hz, 1H), 5.06 (t, J = 6.26 Hz, 1H), 3.21-3.31 (m, 2H), 2.16 (s, 3H). |
| | N-(1-(6-(2-fluoroethyl)pyridin-2-yl)-1H- | |
| | pyrazolo[4,3-c]pyridin-6-yl)acetamide | |
| 631 | 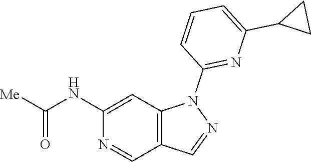 [SMILES: CC(=O)Nc1cc2c(cn1)cnn2-c1cccc(C2CC2)n1] | LCMS m/z = 294 [M + H]⁺ 1H NMR (500 MHz, DMSO-d₆) δ 10.65 (s, 1H), 9.21 (s, 1H), 8.93 (d, J = 1.07 Hz, 1H), 8.50 (d, J = 0.76 Hz, 1H), 7.81-7.91 (m, 1H), 7.70 (dd, J = 0.69, 8.16 Hz, 1H), 7.29 (dd, J = 0.69, 7.55 Hz, 1H), 2.11- 2.26 (m, 4H), 1.26-1.30 (m, 2H), 1.02-1.07 (m, 2H). |
| | N-(1-(6-cyclopropylpyridin-2-y1)-1H- | |
| | pyrazolo[4,3-c]pyridin-6-yl)acetamide | |
| 632 | 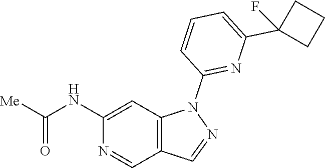 [SMILES: CC(=O)Nc1cc2c(cn1)cnn2-c1cccc(C2(F)CCC2)n1] | LCMS m/z = 321 [M + H]⁺ 1H NMR (400 MHz, CDCl₃) δ 9.77 (br s, 1H), 9.66 (s, 1H), 8.82 (d, J = 1.00 Hz, 1H), 8.39 (d, J = 0.75 Hz, 1H), 7.96-8.01 (m, 1H), 7.90- 7.95 (m, 1H), 7.49 (td, J = 1.13, 7.53 Hz, 1H), 2.94-3.09 (m, 2H), 2.60-2.78 (m, 2H), 2.33 (s, 3H), 2.19-2.29 (m, 2H) |
| | N-(1-(6-(1-fluorocyclobutyl)pyridin-2-yl)- | |
| | 1H-pyrazolo[4,3-c]pyridin-6-yl)acetamide | |
| 633 | 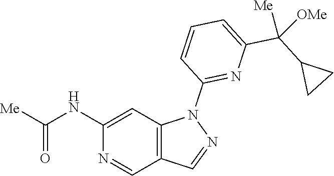 [SMILES: COC(C)(c1cccc(-n2ncc3cnc(NC(C)=O)cc32)n1)C1CC1] | LCMS m/z = 374 [M + Na]⁺ 1H NMR (400 MHz, CDCl₃) δ 9.53 (s, 1H), 9.07 (br s, 1H), 8.79 (d, J = 1.00 Hz, 1H), 8.32 (d, J = 0.75 Hz, 1H), 7.84-7.97 (m, 2H), 7.51- 7.57 (m, 1H), 3.27 (s, 3H), 2.32 (s, 3H), 1.82 (s, 3H), 1.30-1.34 (m, 1H), 0.53-0.64 (m, 2H), 0.40-0.51 (m, 2H). |
| | N-(1-(6-(1-cyclopropyl-1- | |
| | methoxyethyl)pyridin-2-y1)-1H- | |
| | pyrazolo[4,3-c]pyridin-6-yl)acetamide | |
| 634 | 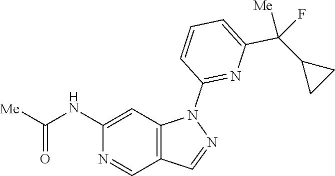 [SMILES: CC(=O)Nc1cc2c(cn1)cnn2-c1cccc(C(C)(F)C2CC2)n1] | LCMS m/z = 340 [M + H]⁺ 1H NMR (400 MHz, CDCl₃) δ 9.50 (s, 1H), 8.79 (d, J = 1.00 Hz, 1H), 8.41 (br s, 1H), 8.26 (s, 1H), 7.93-7.98 (m, 1H), 7.87-7.92 (m, 1H), 7.39- 7.46 (m, 1H), 2.29 (s, 3H), 1.95- 2.04 (m, 3H), 1.69-1.81 (m, 1H), 0.69-0.76 (m, 1H), 0.64 (dt, J = 4.27, 9.16 Hz, 1H), 0.51-0.59 (m, 1H), 0.39-0.47 (m, 1H) |
| | N-(1-(6-(1-cyclopropyl-1- | |
| | fluoroethyl)pyridin-2-yl)-1H- | |
| | pyrazolo[4,3-c]pyridin-6-yl)acetamide | |
| 635 | 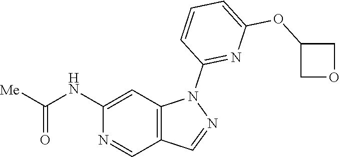 [SMILES: CC(=O)Nc1cc2c(cn1)cnn2-c1cccc(OC2COC2)n1] | LCMS m/z = 326 [M + H]⁺ 1H NMR (500 MHz, DMSO-d₆) δ 10.77 (s, 1H), 9.22 (s, 1H), 8.95 (d, J = 1.07 Hz, 1H), 8.53 (d, J = 0.76 Hz, 1H), 7.97 (t, J = 7.93 Hz, 1H), 7.57-7.61 (m, 1H), 6.85-6.89 (m, 1H), 6.01-6.07 (m, 1H), 5.03-5.09 (m, 2H), 4.58 (ddd, J = 0.69, 4.54, 7.44 Hz, 2H), 2.21 (s, 3H). |
| | N-(1-(6-(oxetan-3-yloxy)pyridin-2-yl)- | |
| | 1H-pyrazolo[4,3-c]pyridin-6-yl)acetamide | |
| 636 | 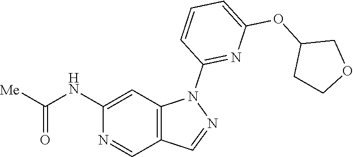 [SMILES: CC(=O)Nc1cc2c(cn1)cnn2-c1cccc(OC2CCOC2)n1] | LCMS m/z = 340 [M + H]⁺ 1H NMR (500 MHz, DMSO-d₆) δ 10.73 (s, 1H), 9.32 (s, 1H), 8.95 (d, J = 1.07 Hz, 1H), 8.54 (d, J = 0.76 Hz, 1H), 7.91 (t, J = 8.01 Hz, 1H), 7.57 (d, J = 7.48 Hz, 1H), 6.75 (d, J = 7.63 Hz, 1H), 5.96-6.07 (m, 1H), 4.04 (dd, J = 4.58, 10.22 Hz, 1H), 3.84-3.92 (m, 2H), 3.81 (dt, J = 4.58, 8.24 Hz, 1H), 2.39 (dtd, |
| | | J = 6.18, 8.04, 13.75 Hz, 1H), 2.15 |
| | N-(1-(6-((tetrahydrofuran-3- | (s, 3H), 2.03-2.10 (m, 1H). |
| | yl)oxy)pyridin-2-yl)-1H-pyrazolo[4,3- | |
| | c]pyridin-6-yl)acetamide | |
| 637 | 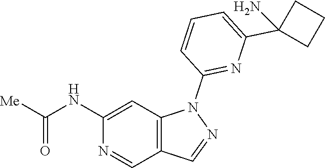 [SMILES: CC(=O)Nc1cc2c(cn1)cnn2-c1cccc(C2(N)CCC2)n1] | LCMS m/z = 323 [M + H]⁺ 1H NMR (500 MHz, DMSO-d₆) δ 10.65 (s, 1H), 9.41 (s, 1H), 8.95 (d, J = 1.07 Hz, 1H), 8.53 (d, J = 0.76 Hz, 1H), 7.92-8.02 (m, 1H), 7.81 (dd, J = 0.69, 8.16 Hz, 1H), 7.51 (dd, J = 0.76, 7.63 Hz, 1H), 2.65- 2.76 (m, 2H), 2.14 (s, 3H), 2.04- 2.12 (m, 3H), 1.86-1.96 (m, 1H). |
| | N-(1-(6-(1-aminocyclobutyl)pyridin-2-yl)- | |
| | 1H-pyrazolo[4,3-c]pyridin-6-yl)acetamide | |
| 638 | 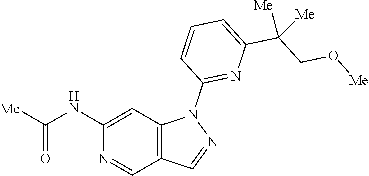 [SMILES: COCC(C)(C)c1cccc(-n2ncc3cnc(NC(C)=O)cc32)n1] | LCMS m/z = 340 [M + H]⁺ |
| | N-(1-(6-(1-methoxy-2-methylpropan-2- | |
| | yl)pyridin-2-yl)-1H-pyrazolo[4,3- | |
| | c]pyridin-6-yl)acetamide | |
| 639 | 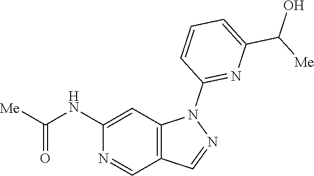 [SMILES: CC(=O)Nc1cc2c(cn1)cnn2-c1cccc(C(C)O)n1] | LCMS m/z = 298 [M + H]⁺ 1H NMR (500 MHz, DMSO-d₆) δ 10.68 (s, 1H), 9.41 (s, 1H), 8.95 (d, J = 1.07 Hz, 1H), 8.53 (d, J = 0.76 Hz, 1H), 8.01 (t, J = 7.93 Hz, 1H), 7.81 (d, J = 8.24 Hz, 1H), 7.48 (d, J = 7.48 Hz, 1H), 4.85 (q, J = 6.56 Hz, 1H), 2.15 (s, 3H), 1.57 (d, J = 6.56 Hz, 3H). |
| | N-(1-(6-(1-hydroxyethyl)pyridin-2-yl)- | |
| | 1H-pyrazolo[4,3-c]pyridin-6-yl)acetamide | |
| 640 | 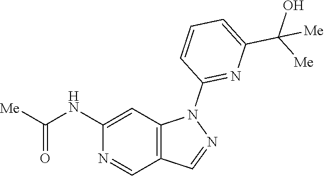 [SMILES: CC(=O)Nc1cc2c(cn1)cnn2-c1cccc(C(C)(C)O)n1] | LCMS m/z = 312 [M + H]⁺ 1H NMR (500 MHz, DMSO-d₆) 10.66 (s, 1H), 9.41 (s, 1H), 8.95 (d, J = 0.92 Hz, 1H), 8.52 (s, 1H), 7.98 (t, J = 7.93 Hz, 1H), 7.81 (d, J = 8.09 Hz, 1H), 7.60 (d, J = 7.63 Hz, 1H), 2.15 (s, 3H), 1.61 (s, 6H). |
| | N-(1-(6-(2-hydroxypropan-2-yl)pyridin-2- | |
| | yl)-1H-pyrazolo[4,3-c]pyridin-6- | |
| | yl)acetamide | |
| 641 | 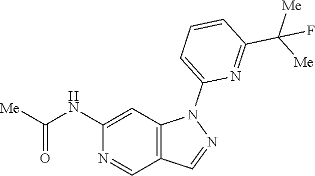 [SMILES: CC(=O)Nc1cc2c(cn1)cnn2-c1cccc(C(C)(C)F)n1] | LCMS m/z = 314 [M + H]⁺ 1H NMR (500 MHz, DMSO-d₆) δ 10.71 (s, 1H), 9.42 (s, 1H), 8.96 (d, J = 1.07 Hz, 1H), 8.55 (d, J = 0.92 Hz, 1H), 8.03-8.13 (m, 1H), 7.93 (dd, J = 0.76, 8.24 Hz, 1H), 7.47 (ddd, J = 0.76, 1.83, 7.63 Hz, 1H), 2.16 (s, 3H), 1.81-1.87 (m, 6H). |
| | N-(1-(6-(2-fluoropropan-2-y1)pyridin-2- | |
| | yl)-1H-pyrazolo[4,3-c]pyridin-6- | |
| | yl)acetamide | |
| 642 | 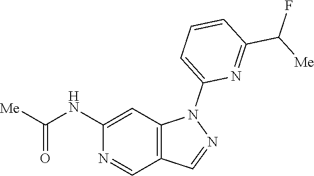 [SMILES: CC(=O)Nc1cc2c(cn1)cnn2-c1cccc(C(C)F)n1] | LCMS m/z = 300 [M + H]⁺ 1H NMR (400 MHz, CDCl₃) δ 12.37 (br s, 1H), 9.96 (s, 1H), 8.77 (s, 1H), 8.47 (s, 1H), 7.95-8.10 (m, 2H), 7.53 (br d, J = 6.53 Hz, 1H), 5.77-5.96 (m, 1H), 2.39 (s, 3H), 1.87-2.01 (m, 3H) |
| | N-(1-(6-(1-fluoroethyl)pyridin-2-yl)-1H- | |
| | pyrazolo[4,3-c]pyridin-6-yl)acetamide | |
| 643 | 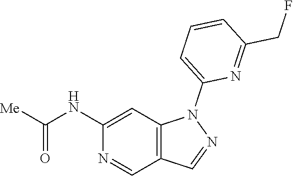 [SMILES: CC(=O)Nc1cc2c(cn1)cnn2-c1cccc(CF)n1] | LCMS m/z = 286 [M + H]⁺ 1H NMR (500 MHz, DMSO-d₆) δ 10.69 (s, 1H), 9.43 (s, 1H), 8.95 (d, J = 1.07 Hz, 1H), 8.56 (d, J = 0.92 Hz, 1H), 8.12 (t, J = 7.93 Hz, 1H), 7.97 (d, J = 8.39 Hz, 1H), 7.47 (d, J = 7.32 Hz, 1H), 5.46-5.71 (m, 2H), 2.15 (s, 3H) |
| | N-(1-(6-(fluoromethyl)pyridin-2-yl)-1H- | |
| | pyrazolo[4,3-c]pyridin-6-yl)acetamide | |
| 644 | 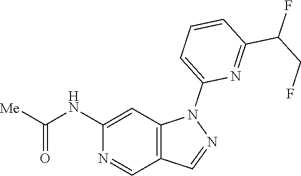 [SMILES: CC(=O)Nc1cc2c(cn1)cnn2-c1cccc(C(F)CF)n1] | LCMS m/z = 318 [M + H]⁺ 1H NMR (400 MHz, CDCl₃) δ 12.19 (br s, 1H), 9.97 (s, 1H), 8.83 (br s, 1H), 8.56 (s, 1H), 8.03-8.11 (m, 2H), 7.64 (d, J = 6.53 Hz, 1H), 5.85-6.09 (m, 1H), 5.06-5.38 (m, 2H), 2.44 (s, 3H) |
| | N-(1-(6-(1,2-difluoroethyl)pyridin-2-yl)- | |
| | 1H-pyrazolo[4,3-c]pyridin-6-yl)acetamide | |
| 645 | 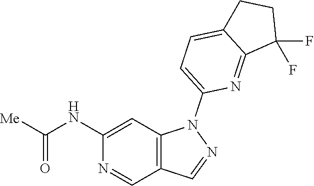 [SMILES: CC(=O)Nc1cc2c(cn1)cnn2-c1ccc2c(n1)C(F)(F)CC2] | LCMS m/z = 330 [M + H]⁺ 1H NMR (400 MHz, MeOD-d₄) δ 9.08 (s, 1H), 9.00 (s, 1H), 8.63 (s, 1H), 8.23 (d, J = 8.28 Hz, 1H), 8.06 (br d, J = 8.53 Hz, 1H), 3.13 (br s, 2H), 2.74 (tt, J = 7.06, 14.40 Hz, 2H), 2.30 (s, 3H) |
| | N-(1-(7,7-difluoro-6,7-dihydro-5H- | |
| | cyclopenta[b]pyridin-2-yl)-1H- | |
| | pyrazolo[4,3-c]pyridin-6-yl)acetamide | |
| 646 | 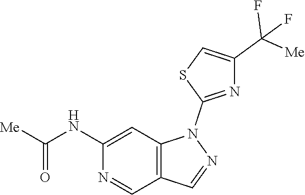 [SMILES: CC(=O)Nc1cc2c(cn1)cnn2-c1nc(C(C)(F)F)cs1] | LCMS m/z = 324 [M + H]⁺ 1H NMR (500 MHz, DMSO-d₆) δ 10.84 (s, 1H), 9.24 (s, 1H), 8.99 (d, J = 1.07 Hz, 1H), 8.61 (d, J = 0.76 Hz, 1H), 7.81-7.87 (m, 1H), 2.05- 2.22 (m, 6H). |
| | N-(1-(4-(1,1-difluoroethyl)thiazol-2-yl)- | |
| | 1H-pyrazolo[4,3-c]pyridin-6-yl)acetamide | |
| 647 | 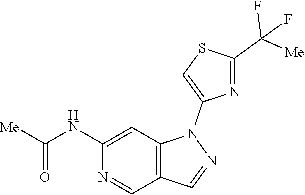 [SMILES: CC(=O)Nc1cc2c(cn1)cnn2-c1csc(C(C)(F)F)n1] | LCMS m/z = 324 [M + H]⁺ 1H NMR (400 MHz, CDCl₃) δ 10.36 (br s, 1H), 9.52 (s, 1H), 8.82 (d, J = 1.00 Hz, 1H), 8.42 (d, J = 0.75 Hz, 1H), 7.71 (s, 1H), 2.31-2.42 (m, 6H) |
| | N-(1-(2-(1,1-difluoroethyl)thiazol-4-yl)- | |
| | 1H-pyrazolo[4,3-c]pyridin-6-yl)acetamide | |
| 648 | 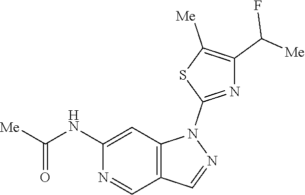 [SMILES: CC(=O)Nc1cc2c(cn1)cnn2-c1nc(C(C)F)c(C)s1] | LCMS m/z = 320 [M + H]⁺ 1H NMR (400 MHz, DMSO-d₆) δ 10.79 (s, 1H), 9.24 (s, 1H), 8.97 (d, J = 0.75 Hz, 1H), 8.55 (s, 1H), 5.76- 5.99 (m, 1H), 2.16 (s, 3H), 1.74- 1.87 (m, 3H) |
| | N-(1-(4-(1-fluoroethyl)-5-methylthiazol- | |
| | 2-y1)-1H-pyrazolo[4,3-c]pyridin-6- | |
| | yl)acetamide | |
| 649 | 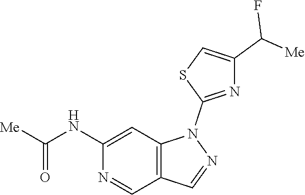 [SMILES: CC(=O)Nc1cc2c(cn1)cnn2-c1nc(C(C)F)cs1] | LCMS m/z = 306 [M + H]⁺ 1H NMR (400 MHz, CDCl₃) δ 9.37 (s, 1H), 8.80 (s, 1H), 8.51 (br s, 1H), 8.25 (d, J = 0.75 Hz, 1H), 7.09 (s, 1H), 5.69-5.94 (m, 1H), 2.30 (s, 3H), 1.83-1.92 (m, 3H) |
| | N-(1-(4-(1-fluoroethyl)thiazol-2-y1)-1H- | |
| | pyrazolo[4,3-c]pyridin-6-yl)acetamide | |
| 650 | 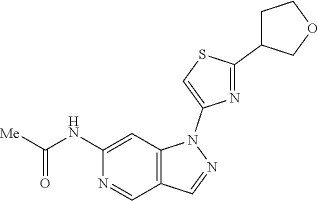 [SMILES: CC(=O)Nc1cc2c(cn1)cnn2-c1csc(C2CCOC2)n1] | LCMS m/z = 330 [M + H]⁺ 1H NMR (400 MHz, MeOD-d₄) δ: 9.11 (s, 1H), 8.85 (d, J = 1.00 Hz, 1H), 8.33 (s, 1H), 7.48 (s, 1H), 4.24 (dd, J = 7.15, 8.41 Hz, 1H), 4.13-4.21 (m, 1H), 4.04-4.10 (m, 1H), 3.94-4.04 (m, 2H), 2.47-2.60 (m, 2H), 2.23 (s, 3H). |
| | N-(1-(2-(tetrahydrofuran-3-yl)thiazol-4- | |
| | yl)-1H-pyrazolo[4,3-c]pyridin-6- | |
| | yl)acetamide | |
| 651 | 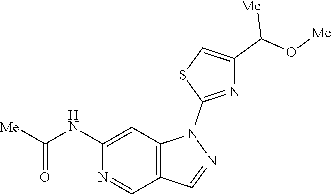 [SMILES: COC(C)c1csc(-n2ncc3cnc(NC(C)=O)cc32)n1] | LCMS m/z = 318 [M + H]⁺ 1H NMR (400 MHz, CDCl₃) δ 9.38 (s, 1H), 8.79 (d, J = 1.00 Hz, 1H), 8.50 (br s, 1H), 8.24 (d, J = 0.75 Hz, 1H), 6.99 (d, J = 0.75 Hz, 1H), 4.56-4.66 (m, 1H), 3.48 (s, 3H), 2.30 (s, 3H), 1.66 (d, J = 6.53 Hz, 3H) |
| | N-(1-(4-(1-methoxyethyl)thiazol-2-y1)- | |
| | 1H-pyrazolo[4,3-c]pyridin-6-yl)acetamide | |
| 652 | 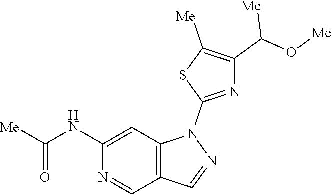 [SMILES: COC(C)c1nc(-n2ncc3cnc(NC(C)=O)cc32)sc1C] | LCMS m/z = 332 [M + H]⁺ 1H NMR (400 MHz, CDCl₃) δ 9.34 (s, 1H), 8.77 (d, J = 1.00 Hz, 1H), 8.55 (br s, 1H), 8.21 (d, J = 0.75 Hz, 1H), 4.64 (q, J = 6.53 Hz, 1H), 3.35 (s, 3H), 2.52 (s, 3H), 2.29 (s, 3H), 1.70 (d, J = 6.53 Hz, 3H). |
| | N-(1-(4-(1-methoxyethyl)-5- | |
| | methylthiazol-2-y1)-1H-pyrazolo[4,3- | |
| | c]pyridin-6-yl)acetamide | |
| 653 | 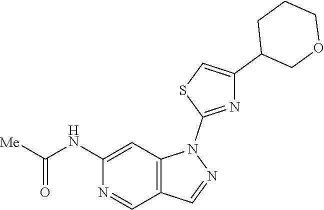 [SMILES: CC(=O)Nc1cc2c(cn1)cnn2-c1nc(C2CCCOC2)cs1] | LCMS m/z = 344 [M + H]⁺. 1H NMR (400 MHz, CDCl₃) δ 9.38 (s, 1H), 8.79 (d, J = 1.00 Hz, 1H), 8.47 (br s, 1H), 8.23 (s, 1H), 6.77 (s, 1H), 4.23 (dd, J = 2.64, 11.17 Hz, 1H), 3.97 (td, J = 3.42, 11.23 Hz, 1H), 3.73 (dd, J = 9.54, 11.04 Hz, 1H), 3.60 (td, J = 6.65, 11.29 Hz, 1H), 3.10-3.19 (m, 1H), 2.30 (s, 3H), 2.20-2.27 (m, 1H), 1.97-2.05 (m, 1H), 1.75-1.83 (m, 2H). |
| | N-(1-(4-(tetrahydro-2H-pyran-3- | |
| | yl)thiazol-2-y1)-1H-pyrazolo[4,3- | |
| | c]pyridin-6-yl)acetamide | |
| 654 | 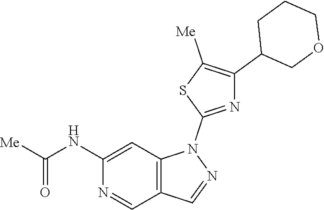 [SMILES: CC(=O)Nc1cc2c(cn1)cnn2-c1nc(C2CCCOC2)c(C)s1] | LCMS m/z = 358 [M + H]⁺ 1H NMR (400 MHz, CDCl₃) δ 9.37 (s, 1H), 8.77 (d, J = 1.00 Hz, 1H), 8.50 (br s, 1H), 8.20 (d, J = 0.75 Hz, 1H), 4.05 (br d, J = 11.04 Hz, 1H), 3.95- 4.01 (m, 1H), 3.80 (t, J = 10.92 Hz, 1H), 3.61-3.72 (m, 1H), 3.05 (tt, J = 4.05, 11.26 Hz, 1H), 2.43 (s, 3H), 2.30 (s, 3H), 2.16-2.26 (m, 1H), 1.98-2.07 (m, 1H), 1.78-1.87 (m, 2H). |
| | N-(1-(5-methyl-4-(tetrahydro-2H-pyran-3- | |
| | yl)thiazol-2-y1)-1H-pyrazolo[4,3- | |
| | c]pyridin-6-yl)acetamide | |
| 655 | 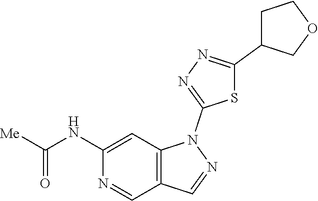 [SMILES: CC(=O)Nc1cc2c(cn1)cnn2-c1nnc(C2CCOC2)s1] | LCMS m/z = 331 [M + H]⁺ 1H NMR (500 MHz, DMSO-d₆) δ 10.86 (s, 1H), 9.09 (s, 1H), 9.00 (d, J = 1.07 Hz, 1H), 8.66 (d, J = 0.92 Hz, 1H), 3.99-4.09 (m, 2H), 3.93- 3.99 (m, 1H), 3.88-3.93 (m, 1H), 3.83 (dt, J = 6.41, 8.24 Hz, 1H), 2.41-2.48 (m, 1H), 2.14-2.25 (m, 4H) |
| | N-(1-(5-(tetrahydrofuran-3-yl)-1,3,4- | |
| | thiadiazol-2-yl)-1H-pyrazolo[4,3- | |
| | c]pyridin-6-yl)acetamide | |
| 656 | 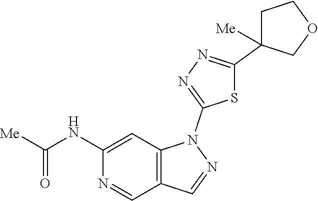 [SMILES: CC(=O)Nc1cc2c(cn1)cnn2-c1nnc(C2(C)CCOC2)s1] | LCMS m/z = 345 [M + H]⁺ 1H NMR (500 MHz, DMSO-d₆) δ 10.86 (s, 1H), 9.10 (s, 1H), 9.00 (d, J = 1.07 Hz, 1H), 8.66 (d, J = 0.92 Hz, 1H), 4.03 (d, J = 8.54 Hz, 1H), 3.91-4.00 (m, 2H), 3.70 (d, J = 8.70 Hz, 1H), 2.42-2.47 (m, 1H), 2.12- 2.22 (m, 4H), 1.60 (s, 3H). |
| | N-(1-(5-(3-methyltetrahydrofuran-3-yl)- | |
| | 1,3,4-thiadiazol-2-yl)-1H-pyrazolo[4,3- | |
| | c]pyridin-6-yl)acetamide | |
| 657 | 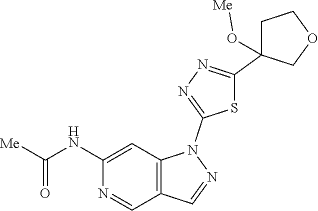 [SMILES: COC1(c2nnc(-n3ncc4cnc(NC(C)=O)cc43)s2)CCOC1] | LCMS m/z = 361 [M + H]⁺ 1H NMR (500 MHz, DMSO-d₆) δ 10.88 (s, 1H), 9.12 (s, 1H), 9.01 (d, J = 1.07 Hz, 1H), 8.69 (d, J = 0.76 Hz, 1H), 4.16 (d, J = 9.77 Hz, 1H), 3.95-4.06 (m, 3H), 3.27 (s, 3H), 2.55-2.66 (m, 2H), 2.17 (s, 3H). |
| | N-(1-(5-(3-methoxytetrahydrofuran-3-yl)- | |
| | 1,3,4-thiadiazol-2-y1)-1H-pyrazolo[4,3- | |
| | c]pyridin-6-yl)acetamide | |
| 658 | 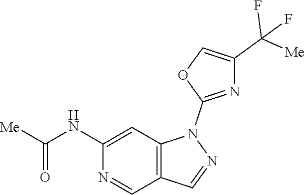 [SMILES: CC(=O)Nc1cc2c(cn1)cnn2-c1nc(C(C)(F)F)co1] | LCMS m/z = 308 [M + H]⁺ 1H NMR (400 MHz, MeOD-d₄) δ 9.00 (s, 1H), 8.86 (s, 1H), 8.57 (d, J = 0.75 Hz, 1H), 8.23 (t, J = 2.01 Hz, 1H), 2.27 (s, 3H), 2.06 (t, J = 18.57 Hz, 3H) |
| | N-(1-(4-(1,1-difluoroethyl)oxazol-2-yl)- | |
| | 1H-pyrazolo[4,3-c]pyridin-6-yl)acetamide | |
| 659 | 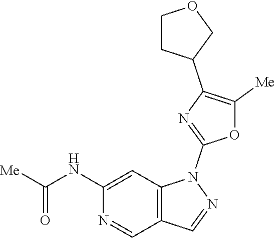 [SMILES: CC(=O)Nc1cc2c(cn1)cnn2-c1nc(C2CCOC2)c(C)o1] | LCMS m/z = 328 [M + H]⁺ 1H NMR (400 MHz, MeOD-d₄) δ 8.99 (s, 1H), 8.89 (d, J = 1.00 Hz, 1H), 8.41 (d, J = 0.75 Hz, 1H), 4.07-4.20 (m, 2H), 3.98 (q, J = 7.28 Hz, 1H), 3.82 (t, J = 8.03 Hz, 1H), 3.48 (quin, J = 8.03 Hz, 1H), 2.42 (s, 3H), 2.25- 2.32 (m, 2H), 2.23 (s, 3H). |
| | N-(1-(5-methyl-4-(tetrahydrofuran-3- | |
| | yl)oxazol-2-y1)-1H-pyrazolo[4,3- | |
| | c]pyridin-6-yl)acetamide | |
| 660 | 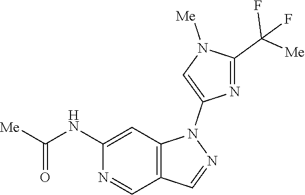 [SMILES: CC(=O)Nc1cc2c(cn1)cnn2-c1cn(C)c(C(C)(F)F)n1] | LCMS m/z = 321 [M + H]⁺ 1H NMR (400 MHz, MeOD-d₄) δ 8.99 (d, J = 1.00 Hz, 1H), 8.64 (s, 1H), 8.47 (d, J = 0.75 Hz, 1H), 7.54 (s, 1H), 3.95 (t, J = 1.13 Hz, 3H), 2.17- 2.31 (m, 6H) |
| | N-(1-(2-(1,1-difluoroethyl)-1-methyl-1H- | |
| | imidazol-4-y1)-1H-pyrazolo[4,3-c]pyridin- | |
| | 6-yl)acetamide | |
| 661 | 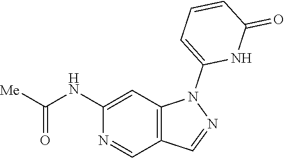 [SMILES: CC(=O)Nc1cc2c(cn1)cnn2-c1cccc(=O)[nH]1] | LCMS m/z = 270 [M + H]⁺ |
| | N-(1-(6-oxo-1,6-dihydropyridin-2-y1)-1H- | |
| | pyrazolo[4,3-c]pyridin-6-yl)acetamide | |
| 662 | 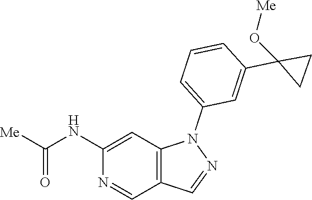 [SMILES: COC1(c2cccc(-n3ncc4cnc(NC(C)=O)cc43)c2)CC1] | LCMS m/z = 323 [M + H]⁺ 1H NMR (500 MHz, DMSO-d₆) δ 10.73 (s, 1H), 8.97 (d, J = 1.07 Hz, 1H), 8.53 (s, 1H), 8.49 (d, J = 0.92 Hz, 1H), 7.56-7.63 (m, 3H), 7.30 (td, J = 1.83, 6.87 Hz, 1H), 3.26 (s, 3H), 2.12 (s, 3H), 1.23-1.27 (m, 2H), 1.08-1.13 (m, 2H). |
| | N-(1-(3-(1-methoxycyclopropyl)phenyl)- | |
| | 1H-pyrazolo[4,3-c]pyridin-6-yl)acetamide | |
| 663 | 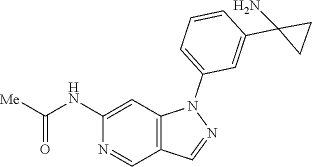 [SMILES: CC(=O)Nc1cc2c(cn1)cnn2-c1cccc(C2(N)CC2)c1] | LCMS m/z = 308 [M + H]⁺ 1H NMR (500 MHz, DMSO-d₆) δ 10.74 (s, 1H), 8.97 (d, J = 0.76 Hz, 1H), 8.50 (s, 2H), 7.68 (t, J = 1.68 Hz, 1H), 7.49-7.65 (m, 2H), 7.44 (td, J = 1.74, 7.21 Hz, 1H), 2.12 (s, 3H), 1.16-1.28 (m, 4H). |
| | N-(1-(3-(1-aminocyclopropyl)phenyl)-1H- | |
| | pyrazolo[4,3-c]pyridin-6-yl)acetamide | |
| 664 | 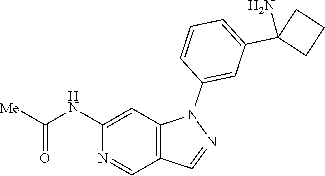 [SMILES: CC(=O)Nc1cc2c(cn1)cnn2-c1cccc(C2(N)CCC2)c1] | LCMS m/z = 322 [M + H]⁺ 1H NMR (500 MHz, DMSO-d₆) δ: 10.71 (s, 1H), 8.96 (d, J = 0.92 Hz, 1H), 8.53 (s, 1H), 8.48 (d, J = 0.92 Hz, 1H), 7.77 (t, J = 1.83 Hz, 1H), 7.49-7.63 (m, 3H), 3.38-3.69 (m, 1H), 2.42-2.48 (m, 2H), 2.09- 2.21 (m, 5H), 1.96-2.07 (m, 1H), 1.66-1.79 (m, 1H). |
| | N-(1-(3-(1-aminocyclobutyl)phenyl)-1H- | |
| | pyrazolo[4,3-c]pyridin-6-yl)acetamide | |
| 665 | 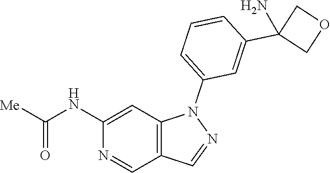 [SMILES: CC(=O)Nc1cc2c(cn1)cnn2-c1cccc(C2(N)COC2)c1] | LCMS m/z = 324 [M + H]⁺ 1H NMR (500 MHz, DMSO-d₆) δ 10.79 (s, 1H), 9.02 (br s, 2H), 8.52-8.55 (m, 1H), 7.91-7.94 (m, 1H), 7.75-7.85 (m, 2H), 7.57-7.62 (m, 1H), 5.01-5.06 (m, 2H), 4.96- 5.01 (m, 2H), 3.40-3.49 (m, 2H), 2.13 (s, 3H). |
| | N-(1-(3-(3-aminooxetan-3-yl)phenyl)-1H- | |
| | pyrazolo[4,3-c]pyridin-6-yl)acetamide | |
| 666 | 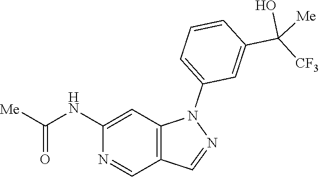 [SMILES: CC(=O)Nc1cc2c(cn1)cnn2-c1cccc(C(C)(O)C(F)(F)F)c1] | LCMS m/z = 365 [M + H]⁺ 1H NMR (500 MHz, DMSO-d₆) δ 10.73 (s, 1H), 8.98 (d, J = 1.07 Hz, 1H), 8.51 (s, 1H), 7.93 (s, 1H), 7.70-7.75 (m, 1H), 7.64-7.70 (m, 2H), 2.09-2.18 (m, 3H), 1.77 (s, 3H). |
| | N-(1-(3-(1,1,1-trifluoro-2-hydroxypropan- | |
| | 2-yl)phenyl)-1H-pyrazolo[4,3-c]pyridin- | |
| | 6-yl)acetamide | |
| 667 | 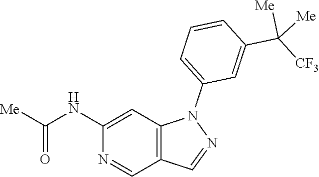 [SMILES: CC(=O)Nc1cc2c(cn1)cnn2-c1cccc(C(C)(C)C(F)(F)F)c1] | LCMS m/z = 363 [M + H]⁺ 1H NMR (500 MHz, DMSO-d₆) δ: 10.74 (s, 1H), 8.98 (d, J = 1.07 Hz, 1H), 8.54 (s, 1H), 8.51 (d, J = 0.92 Hz, 1H), 7.86 (s, 1H), 7.70-7.75 (m, 1H), 7.62-7.70 (m, 2H), 2.12 (s, 3H), 1.64 (s, 6H). |
| | N-(1-(3-(1,1,1-trifluoro-2-methylpropan- | |
| | 2-y1)phenyl)-1H-pyrazolo[4,3-c]pyridin- | |
| | 6-yl)acetamide | |
| 668 | 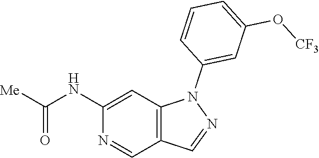 [SMILES: CC(=O)Nc1cc2c(cn1)cnn2-c1cccc(OC(F)(F)F)c1] | LCMS m/z = 337 [M + H]⁺ 1H NMR (500 MHz, DMSO-d₆) δ 10.77 (s, 1H), 8.99 (d, J = 1.07 Hz, 1H), 8.52-8.61 (m, 1H), 7.75-7.82 (m, 2H), 7.73 (d, J = 0.76 Hz, 1H), 7.42-7.50 (m, 1H), 2.13 (s, 3H). |
| | N-(1-(3-(trifluoromethoxy)phenyl)-1H- | |
| | pyrazolo[4,3-c]pyridin-6-yl)acetamide | |
| 669 | 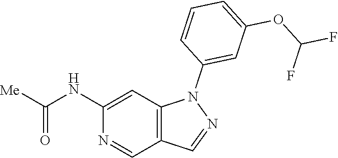 [SMILES: CC(=O)Nc1cc2c(cn1)cnn2-c1cccc(OC(F)F)c1] | LCMS m/z = 319 [M + H]⁺ 1H NMR (500 MHz, DMSO-d₆) δ 10.76 (s, 1H), 8.98 (d, J = 0.92 Hz, 1H), 8.53 (s, 1H), 7.67-7.72 (m, 1H), 7.59-7.65 (m, 1H), 7.54 (t, J = 2.14 Hz, 1H), 7.22-7.53 (m, 2H), 2.13 (s, 3H). |
| | N-(1-(3-(difluoromethoxy)phenyl)-1H- | |
| | pyrazolo[4,3-c]pyridin-6-yl)acetamide | |
| 670 | 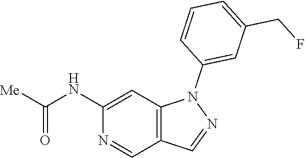 [SMILES: CC(=O)Nc1cc2c(cn1)cnn2-c1cccc(CF)c1] | LCMS m/z = 285 [M + H]⁺ 1H NMR (500 MHz, DMSO-d₆) δ 10.74 (s, 1H), 8.98 (d, J = 0.92 Hz, 1H), 8.48-8.56 (m, 1H), 7.77 (s, 1H), 7.71-7.75 (m, 1H), 7.66-7.71 (m, 1H), 7.50 (br d, J = 7.32 Hz, 1H), 5.45-5.67 (m, 2H), 2.12 (s, 3H). |
| | N-(1-(3-(fluoromethyl)phenyl)-1H- | |
| | pyrazolo[4,3-c]pyridin-6-yl)acetamide | |
| 671 | 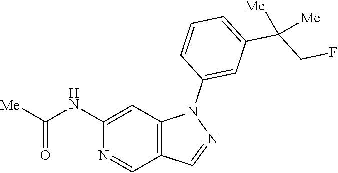 [SMILES: CC(=O)Nc1cc2c(cn1)cnn2-c1cccc(C(C)(C)CF)c1] | LCMS m/z = 327 [M + H]⁺ 1H NMR (500 MHz, DMSO-d₆) δ 10.71 (s, 1H), 8.97 (s, 1H), 8.32- 8.65 (m, 1H), 7.47-7.68 (m, 3H), 7.31 (d, J = 7.32 Hz, 1H), 2.96-3.11 (m, 2H), 2.12 (s, 3H), 1.38 (s, 3H), 1.33 (s, 3H). |
| | N-(1-(3-(1-fluoro-2-methylpropan-2- | |
| | yl)phenyl)-1H-pyrazolo[4,3-c]pyridin-6- | |
| | yl)acetamide | |
| 672 | 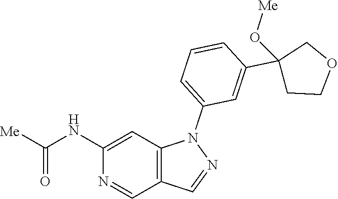 [SMILES: COC1(c2cccc(-n3ncc4cnc(NC(C)=O)cc43)c2)CCOC1] | LCMS m/z = 353 [M + H]⁺ |
| | N-(1-(3-(3-methoxytetrahydrofuran-3- | |
| | yl)phenyl)-1H-pyrazolo[4,3-c]pyridin-6- | |
| | yl)acetamide | |
| 673 | 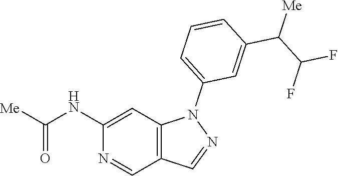 [SMILES: CC(=O)Nc1cc2c(cn1)cnn2-c1cccc(C(C)C(F)F)c1] | LCMS m/z = 331 [M + H]+ ¹H NMR (500 MHz, DMSO-d₆) δ 10.73 (s, 1H), 8.97 (d, J = 1.07 Hz, 1H), 8.51 (s, 1H), 8.50 (d, J = 0.92 Hz, 1H), 7.70 (d, J = 1.68 Hz, 1H), 7.56-7.67 (m, 2H), 7.37-7.50 (m, 1H), 6.11-6.40 (m, 1H), 3.44-3.84 (m, 1H), 2.12 (s, 3H), 1.39 (d, J = 7.17 Hz, 3H). |
| | N-(1-(3-(1,1-difluoropropan-2-y1)phenyl)- | |
| | 1H-pyrazolo[4,3-c]pyridin-6-yl)acetamide | |
| 674 | 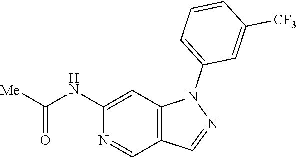 [SMILES: CC(=O)Nc1cc2c(cn1)cnn2-c1cccc(C(F)(F)F)c1] | LCMS m/z = 321 [M + H]⁺ 1H NMR (500 MHz, DMSO-d₆) δ 10.79 (s, 1H), 9.00 (d, J = 0.76 Hz, 1H), 8.56 (s, 1H), 8.01-8.11 (m, 2H), 7.89 (t, J = 7.86 Hz, 1H), 7.79- 7.85 (m, 1H), 2.13 (s, 3H). |
| | N-(1-(3-(trifluoromethyl)phenyl)-1H- | |
| | pyrazolo[4,3-c]pyridin-6-yl)acetamide | |
| 675 | 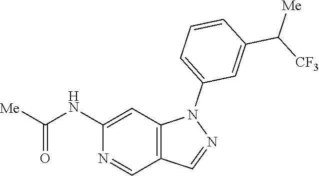 [SMILES: CC(=O)Nc1cc2c(cn1)cnn2-c1cccc(C(C)C(F)(F)F)c1] | LCMS m/z = 349 [M + H]⁺ 1H NMR (400 MHz, CDCl₃) δ 9.22 (br s, 1H), 8.83 (s, 1H), 8.70 (s, 1H), 8.35 (d, J = 0.75 Hz, 1H), 7.67- 7.74 (m, 2H), 7.58 (t, J = 8.16 Hz, 1H), 7.40 (d, J = 7.78 Hz, 1H), 3.52- 3.66 (m, 1H), 2.29 (s, 3H), 1.62 (d, J = 7.28 Hz, 3H) |
| | N-(1-(3-(1,1,1-trifluoropropan-2- | |
| | yl)phenyl)-1H-pyrazolo[4,3-c]pyridin-6- | |
| | yl)acetamide | |
| 676 | 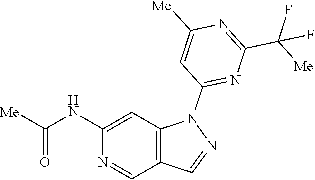 [SMILES: CC(=O)Nc1cc2c(cn1)cnn2-c1cc(C)nc(C(C)(F)F)n1] | LCMS m/z = 333 [M + H]⁺ 1H NMR (MeOD-d₄, 400 MHz): δ (ppm) 9.47 (s, 1H), 8.86-8.93 (m, 1H), 8.46 (d, J = 1.0 Hz, 1H), 8.00 (s, 1H), 2.67 (s, 3H), 2.19-2.31 (m, 6H) |
| | N-(1-(2-(1,1-difluoroethyl)-6- | |
| | methylpyrimidin-4-yl)-1H-pyrazolo[4,3- | |
| | c]pyridin-6-yl)acetamide | |
| 677 | 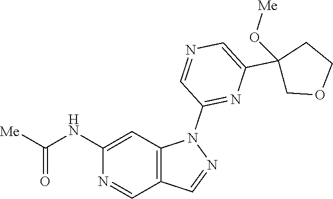 [SMILES: COC1(c2cncc(-n3ncc4cnc(NC(C)=O)cc43)n2)CCOC1] | LCMS m/z = 355 [M + H]⁺ |
| | N-(1-(6-(3-methoxytetrahydrofuran-3- | |
| | yl)pyrazin-2-yl)-1H-pyrazolo[4,3- | |
| | c]pyridin-6-yl)acetamide | |
| 678 | 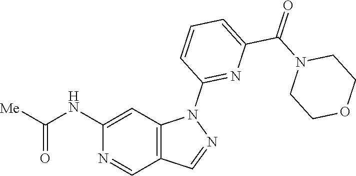 [SMILES: CC(=O)Nc1cc2c(cn1)cnn2-c1cccc(C(=O)N2CCOCC2)n1] | LCMS m/z = 367.0 [M + H]⁺ 1H NMR(500 MHz, DMSO-d₆) (ppm) 10.70 (s, 1H), 9.36 (s, 1H), 8.96 (s, 1H), 8.57 (s, 1H), 8.15 (t, J = 7.9, 7.9 Hz, 1H), 8.07 (d, J = 8.3 Hz, 1H), 7.53 (d, J = 7.4 Hz, 1H), 3.83-3.75 (m, 4H), 3.60-3.53 (m, 4H), 2.17 (s, 3H) |
| | N-(1-(6-(morpholine-4-carbonyl)pyridin- | |
| | 2-y1)-1H-pyrazolo[4,3-c]pyridin-6- | |
| | yl)acetamide | |
| 679 | 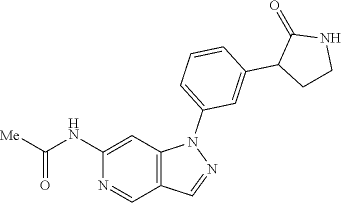 [SMILES: CC(=O)Nc1cc2c(cn1)cnn2-c1cccc(C2CCNC2=O)c1] | LCMS m/z = 336.1 [M + H]⁺ 1H NMR (600 MHz, DMSO-d₆) ppm 10.72 (s, 1H), 8.97 (s, 1H), 8.54- 8.47 (m, 2H), 7.89 (s, 1H), 7.65- 7.54 (m, 3H), 7.38-7.31 (m, 1H), 3.70 (t, J = 9.0, 9.0 Hz, 1H), 3.41- 3.33 (m, 2H), 2.61-2.56 (m, 1H), 2.23-2.16 (m, 1H), 2.12 (s, 3H) |
| | N-(1-(3-(2-oxopyrrolidin-3-yl)phenyl)- | |
| | 1H-pyrazolo[4,3-c]pyridin-6-yl)acetamide | |
| 680 | 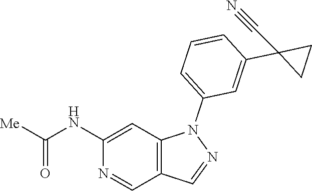 [SMILES: CC(=O)Nc1cc2c(cn1)cnn2-c1cccc(C2(C#N)CC2)c1] | LCMS m/z = 318.1 [M + H]⁺ 1H NMR (600 MHz, DMSO-d₆) ppm 10.67 (s, 1H), 9.36 (s, 1H), 8.91 (s, 1H), 8.50 (s, 1H), 7.88 (t, J = 7.9, 7.9 Hz, 1H), 7.52 (d, J = 7.6 Hz, 1H), 6.74 (d, J = 7.8 Hz, 1H), 4.68-4.65 (m, 2H), 3.76-3.73 (m, 2H), 3.30 (s, 3H), 2.13 (s, 3H) |
| | N-(1-(3-(1-cyanocyclopropyl)phenyl)-1H- | |
| | pyrazolo[4,3-c]pyridin-6-yl)acetamide | |
| 681 | 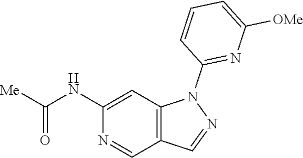 [SMILES: COc1cccc(-n2ncc3cnc(NC(C)=O)cc32)n1] | LCMS m/z = 284.2 [M + H]⁺ |
| | N-(1-(6-methoxypyridin-2-yl)-1H- | |
| | pyrazolo[4,3-c]pyridin-6-yl)acetamide | |
| 682 | 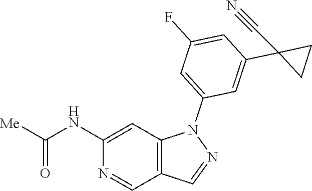 [SMILES: CC(=O)Nc1cc2c(cn1)cnn2-c1cc(F)cc(C2(C#N)CC2)c1] | LCMS m/z = 336.2 [M + H]⁺ 1H NMR (600 MHz, DMSO-d₆) ppm 10.75 (s, 1H), 8.95 (s, 1H), 8.55-8.50 (m, 2H), 7.55-7.50 (m, 2H), 7.22 (d, J = 9.5 Hz, 1H), 2.11 (s, 3H), 1.88-1.85 (m, 2H), 1.71- 1.68 (m, 2H) |
| | N-(1-(3-(1-cyanocyclopropyl)-5- | |
| | fluorophenyl)-1H-pyrazolo[4,3-c]pyridin- | |
| | 6-yl)acetamide | |
| 683 | 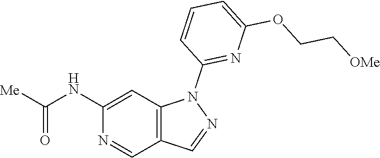 [SMILES: COCCOc1cccc(-n2ncc3cnc(NC(C)=O)cc32)n1] | LCMS m/z = 328.2 [M + H]⁺ 1H NMR (600 MHz, DMSO-d₆) ppm 10.67 (s, 1H), 9.36 (s, 1H), 8.91 (s, 1H), 8.50 (s, 1H), 7.88 (t, J = 7.9, 7.9 Hz, 1H), 7.52 (d, J = 7.6 Hz, 1H), 6.74 (d, J = 7.8 Hz, 1H), 4.68- 4.65 (m, 2H), 3.76-3.73 (m, 2H), 3.30 (s, 3H), 2.13 (s, 3H) |
| | N-(1-(6-(2-methoxyethoxy)pyridin-2-yl)- | |
| | 1H-pyrazolo[4,3-c]pyridin-6-yl)acetamide | |
| 684 | 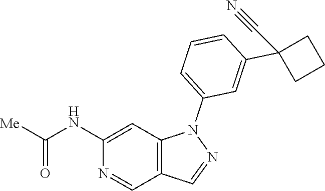 [SMILES: CC(=O)Nc1cc2c(cn1)cnn2-c1cccc(C2(C#N)CCC2)c1] | LCMS m/z = 332.2 [M + H]⁺ 1H NMR(400 MHz, CDCl₃): δ (ppm) 8.84 (s, 1H), 8.64 (s, 1H), 8.46 (s, 1H), 8.27 (s, 1H), 7.83 (s, 1H), 7.72 (d, J = 8.0 Hz, 1H), 7.60 (t, J = 7.9, 7.9 Hz, 1H), 7.47 (d, J = 7.7 Hz, 1H), 2.98-2.88 (m, 2H), 2.81- 2.71 (m, 2H), 2.58-2.42 (m, 1H), 2.26 (s, 3H), 2.22-2.11 (m, 1H) |
| | N-(1-(3-(1-cyanocyclobutyl)phenyl)-1H- | |
| | pyrazolo[4,3-c]pyridin-6-yl)acetamide | |
| 685 | 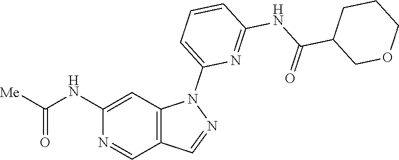 [SMILES: CC(=O)Nc1cc2c(cn1)cnn2-c1cccc(NC(=O)C2CCCOC2)n1] | LCMS m/z = 381.2 [M + H]⁺ 1H NMR (600 MHz, DMSO-d₆) ppm 10.61 (s, 1H), 10.38 (s, 1H), 9.02 (s, 1H), 8.92 (s, 1H), 8.49 (s, 1H), 7.97 (t, J = 8.0, 8.0 Hz, 1H), 7.93 (d, J = 8.0 Hz, 1H), 7.55 (d, J = 7.7 Hz, 1H), 4.04-3.93 (m, 1H), 3.79 (d, J = 11.0 Hz, 1H), 3.43 (t, J = 10.5, 10.5 Hz, 1H), 3.37-3.34 (m, 1H), 2.88-2.81 (m, 1H), 2.12 |
| | | (s, 3H), 2.01-1.95 (m, 1H), 1.77- |
| | N-(6-(6-acetamido-1H-pyrazolo[4,3- | 1.70 (m, 1H), 1.66-1.60 (m, 1H), |
| | c]pyridin-1-yl)pyridin-2-yl)tetrahydro-2H- | 1.58-1.51 (m, 1H) |
| | pyran-3-carboxamide | |
| 686 | 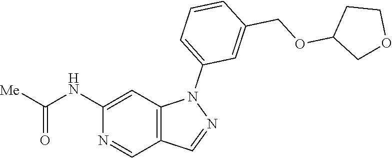 [SMILES: CC(=O)Nc1cc2c(cn1)cnn2-c1cccc(COC2CCOC2)c1] | LCMS m/z = 353.2 [M + H]⁺ |
| | N-(1-(3-(((tetrahydrofuran-3- | |
| | yl)oxy)methyl)phenyl)-1H-pyrazolo[4,3- | |
| | c]pyridin-6-yl)acetamide | |
| 687 | 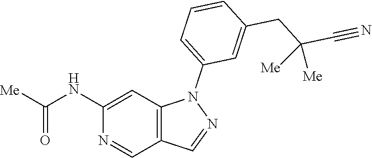 [SMILES: CC(=O)Nc1cc2c(cn1)cnn2-c1cccc(CC(C)(C)C#N)c1] | LCMS m/z = 334.2 [M + H]⁺ 1H NMR (400 MHz, CDCl₃) ppm 8.83 (s, 1H), 8.60 (s, 1H), 8.52 (s, 1H), 8.26 (s, 1H), 7.69 (d, J = 8.1 Hz, 1H), 7.63 (s, 1H), 7.55 (t, J = 7.8, 7.8 Hz, 1H), 7.37 (d, J = 7.4 Hz, 1H), 2.95 (s, 2H), 2.25 (s, 3H), 1.44 (s, 6H) |
| | N-(1-(3-(2-cyano-2- | |
| | methylpropyl)phenyl)-1H-pyrazolo[4,3- | |
| | c]pyridin-6-yl)acetamide | |
| 688 | 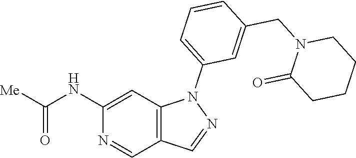 [SMILES: CC(=O)Nc1cc2c(cn1)cnn2-c1cccc(CN2CCCCC2=O)c1] | LCMS m/z = 364.2 [M + H]⁺ 1H NMR(400 MHz, DMSO-d₆ + CCl₄) (ppm) 10.57 (s, 1H), 8.85 (s, 1H), 8.54 (s, 1H), 8.30 (s, 1H), 7.63-7.52 (m, 3H), 7.29 (d, J = 7.3 Hz, 1H), 4.61 (s, 2H), 3.42-3.31 (m, 2H), 2.35 (t, J = 5.8, 5.8 Hz, 2H), 2.13 (s, 3H), 1.90-1.77 (m, 4H) |
| | N-(1-(3-((2-oxopiperidin-1- | |
| | yl)methyl)phenyl)-1H-pyrazolo[4,3- | |
| | c]pyridin-6-yl)acetamide | |
| 689 | 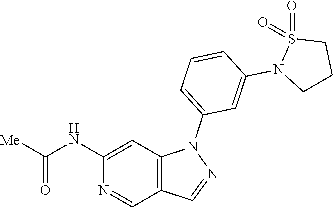 [SMILES: CC(=O)Nc1cc2c(cn1)cnn2-c1cccc(N2CCCS2(=O)=O)c1] | LCMS m/z = 372.0 [M + H]⁺ 1H NMR (400 MHz, CDCl₃) ppm 9.32-8.60 (m, 1H), 8.55-8.03 (m, 2H), 7.62-7.50 (m, 3H), 7.44 -7.36 (m, 1H), 7.30-7.28 (m, 1H), 3.92 (t, J = 6.2, 6.2 Hz, 2H), 3.46 (t, J = 7.4, 7.4 Hz, 2H), 2.68- 2.55 (m, 2H), 2.30 (s, 3H) |
| | N-(1-(3-(1,1-dioxidoisothiazolidin-2- | |
| | yl)phenyl)-1H-pyrazolo[4,3-c]pyridin-6- | |
| | yl)acetamide | |
| 690 | 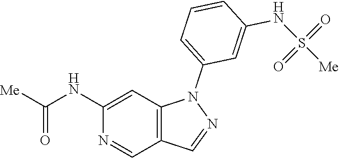 [SMILES: CC(=O)Nc1cc2c(cn1)cnn2-c1cccc(NS(C)(=O)=O)c1] | LCMS m/z = 346.0 [M + H]⁺ |
| | N-(1-(3-(methylsulfonamido)phenyl)-1H- | |
| | pyrazolo[4,3-c]pyridin-6-yl)acetamide | |
| 691 | 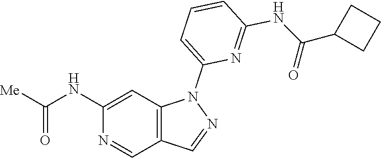 [SMILES: CC(=O)Nc1cc2c(cn1)cnn2-c1cccc(NC(=O)C2CCC2)n1] | LCMS m/z = 351.2 [M + H]⁺ |
| | N-(6-(6-acetamido-1H-pyrazolo[4,3- | |
| | c]pyridin-1-yl)pyridin-2- | |
| | yl)cyclobutanecarboxamide | |
| 692 | 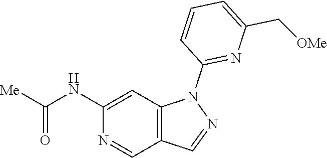 [SMILES: COCc1cccc(-n2ncc3cnc(NC(C)=O)cc32)n1] | LCMS m/z = 298.0 [M + H]⁺ |
| | N-(1-(6-(methoxymethyl)pyridin-2-yl)- | |
| | 1H-pyrazolo[4,3-c]pyridin-6-yl)acetamide | |
| 693 | 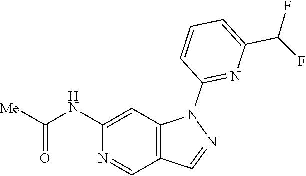 [SMILES: CC(=O)Nc1cc2c(cn1)cnn2-c1cccc(C(F)F)n1] | LCMS m/z = 304.2 [M + H]⁺ 1H NMR(400 MHz, CDCl₃) (ppm) 9.98-9.25 (m, 1H), 8.55-8.18 (m, 2H), 8.15 (d, J = 8.6 Hz, 1H), 8.02 (t, J = 7.8, 7.8 Hz, 1H), 7.57 (d, J = 7.2 Hz, 1H), 7.27-7.25 (m, 1H), 6.85 (t, J = 55.1, 55.1 Hz, 1H), 2.36 (s, 3H) |
| | N-(1-(6-(difluoromethyl)pyridin-2-y1)-1H- | |
| | pyrazolo[4,3-c]pyridin-6-yl)acetamide | |
| 694 | 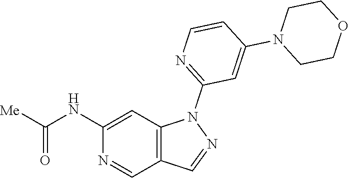 [SMILES: CC(=O)Nc1cc2c(cn1)cnn2-c1cc(N2CCOCC2)ccn1] | LCMS m/z = 339.2 [M + H]⁺ |
| | N-(1-(4-morpholinopyridin-2-yl)-1H- | |
| | pyrazolo[4,3-c]pyridin-6-yl)acetamide | |
| 695 | 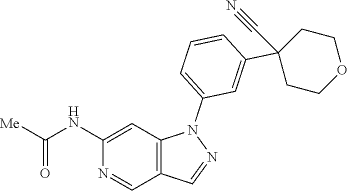 [SMILES: CC(=O)Nc1cc2c(cn1)cnn2-c1cccc(C2(C#N)CCOCC2)c1] | LCMS m/z = 362 [M + H]+ |
| | N-(1-(3-(4-cyanotetrahydro-2H-pyran-4- | |
| | yl)phenyl)-1H-pyrazolo[4,3-c]pyridin-6- | |
| | yl)acetamide | |
| 696 | 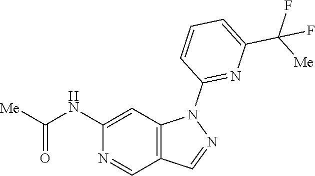 [SMILES: CC(=O)Nc1cc2c(cn1)cnn2-c1cccc(C(C)(F)F)n1] | LCMS m/z = 318.2 [M + H]⁺ |
| | N-(1-(6-(1,1-difluoroethyl)pyridin-2-yl)- | |
| | 1H-pyrazolo[4,3-c]pyridin-6-yl)acetamide | |
| 697 | 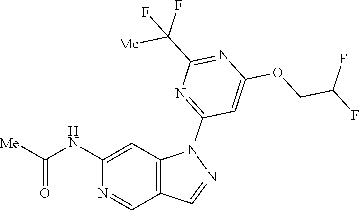 [SMILES: CC(=O)Nc1cc2c(cn1)cnn2-c1cc(OCC(F)F)nc(C(C)(F)F)n1] | LCMS m/z = 399.3 [M + H]⁺ 1H NMR (600 MHz, DMSO-d₆) (ppm) = 10.76 (s, 1H), 9.48-9.34 (m, 1H), 9.00-8.93 (m, 1H), 8.70- 8.63 (m, 1H), 7.44-7.36 (s, 1H), 6.57-6.31 (m, 1H), 4.79 (br d, J = 3.3 Hz, 2H), 2.28-2.19 (m, 3H), 2.15 (s, 3H) |
| | N-(1-(6-(2,2-difluoroethoxy)-2-(1,1- | |
| | difluoroethyl)pyrimidin-4-yl)-1H- | |
| | pyrazolo[4,3-c]pyridin-6-yl)acetamide | |
| 698 | 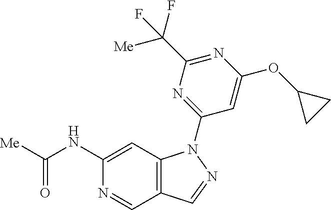 [SMILES: CC(=O)Nc1cc2c(cn1)cnn2-c1cc(OC2CC2)nc(C(C)(F)F)n1] | LCMS m/z = 375.3 [M + H]⁺ 1H NMR (600 MHz, DMSO-d₆) (ppm) = 10.76 (s, 1H), 9.41 (s, 1H), 8.97 (s, 1H), 8.66 (s, 1H), 7.49 (s, 1H), 4.41 (td, J = 3.0, 5.9 Hz, 1H), 2.22 (s, 3H), 2.15 (s, 3H), 0.98-0.65 (m, 4H) |
| | N-(1-(6-cyclopropoxy-2-(1,1- | |
| | difluoroethyl)pyrimidin-4-yl)-1H- | |
| | pyrazolo[4,3-c]pyridin-6-yl)acetamide | |
| 699 | 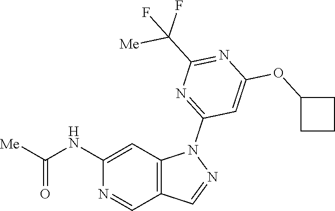 [SMILES: CC(=O)Nc1cc2c(cn1)cnn2-c1cc(OC2CCC2)nc(C(C)(F)F)n1] | LCMS m/z = 388.1 [M + H]⁺ 1H NMR (600 MHz, DMSO-d₆) (ppm) 10.74 (s, 1H), 9.43-9.33 (m, 1H), 9.00-8.91 (m, 1H), 8.68-8.62 (m, 1H), 7.28-7.20 (m, 1H), 5.28 (t, J = 7.4 Hz, 1H), 2.49-2.40 (m, 3H), 2.29-2.06 (m, 9H), 1.87-1.77 (m, 1H), 1.76-1.64 (m, 1H) |
| | N-(1-(6-cyclobutoxy-2-(1,1- | |
| | difluoroethyl)pyrimidin-4-yl)-1H- | |
| | pyrazolo[4,3-c]pyridin-6-yl)acetamide | |
| 700 | 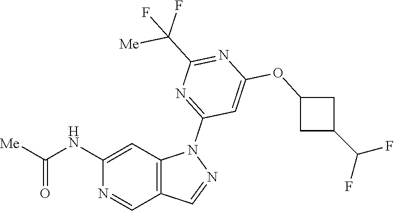 [SMILES: CC(=O)Nc1cc2c(cn1)cnn2-c1cc(OC2CC(C(F)F)C2)nc(C(C)(F)F)n1] | LCMS m/z = 439.2 [M + H]+ 1H NMR (600 MHz, DMSO-d₆) (ppm) 10.72 (s, 1H), 9.36 (s, 1H), 8.98-8.88 (m, 1H), 8.66-8.54 (m, 1H), 7.26-7.20 (m, 1H), 6.09 (s, 1H), 5.38-5.18 (m, 1H), 2.61-2.52 (m, 3H), 2.43-2.32 (m, 1H), 2.28- 2.02 (m, 9H) |
| | N-(1-(2-(1,1-difluoroethyl)-6-(3- | |
| | (difluoromethyl)cyclobutoxy)pyrimidin-4- | |
| | yl)-1H-pyrazolo[4,3-c]pyridin-6- | |
| | yl)acetamide | |
| 701 | 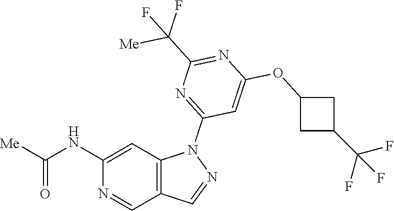 [SMILES: CC(=O)Nc1cc2c(cn1)cnn2-c1cc(OC2CC(C(F)(F)F)C2)nc(C(C)(F)F)n1] | LCMS m/z = 457.2 [M + H]⁺ 1H NMR (600 MHz, DMSO-d₆) (ppm) 10.75 (s, 1H), 9.44-9.33 (m, 1H), 9.01-8.91 (m, 1H), 8.70-8.62 (m, 1H), 7.31-7.22 (m, 1H), 5.45- 5.35 (m, 1H), 5.34-5.23 (m, 1H), 3.12-2.97 (m, 1H), 2.82-2.70 (m, 1H), 2.67 (s, 4H), 2.31-2.13 (m, 7H) |
| | N-(1-(2-(1,1-difluoroethyl)-6-(3- | |
| | (trifluoromethyl)cyclobutoxy)pyrimidin- | |
| | 4-yl)-1H-pyrazolo[4,3-c]pyridin-6- | |
| | yl)acetamide | |
| 702 | 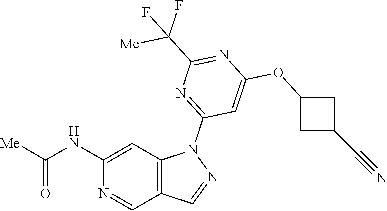 [SMILES: CC(=O)Nc1cc2c(cn1)cnn2-c1cc(OC2CC(C#N)C2)nc(C(C)(F)F)n1] | LCMS m/z = 414.2 [M + H]⁺ 1H NMR (600 MHz, DMSO-d₆) (ppm) 10.75 (s, 1H), 9.44-9.31 (m, 1H), 9.01-8.92 (m, 1H), 8.69-8.60 (m, 1H), 7.34-7.21 (m, 1H), 5.28 (t, J = 7.3 Hz, 1H), 3.19 (s, 1H), 2.97-2.86 (m, 2H), 2.53-2.51 (m, 1H), 2.21 (s, 3H), 2.15 (s, 3H) |
| | N-(1-(6-(3-cyanocyclobutoxy)-2-(1,1- | |
| | difluoroethyl)pyrimidin-4-yl)-1H- | |
| | pyrazolo[4,3-c]pyridin-6-yl)acetamide | |
| 703 | 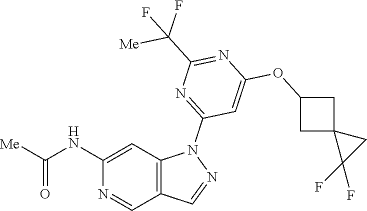 [SMILES: CC(=O)Nc1cc2c(cn1)cnn2-c1cc(OC2CC3(C2)CC3(F)F)nc(C(C)(F)F)n1] | LCMS m/z = 451.2 [M + H]⁺ 1H NMR (600 MHz, DMSO-d₆) (ppm) 10.83-10.66 (m, 1H), 9.45- 9.34 (m, 1H), 9.03-8.91 (m, 1H), 8.69-8.57 (m, 1H), 7.36-7.23 (m, 1H), 5.58-5.47 (m, 1H), 5.46-5.33 (m, 1H), 2.77-2.62 (m, 2H), 2.22 (s, 3H), 2.16 (s, 3H), 1.62-1.53 (m, 1H), 1.52-1.44 (m, 1H) |
| | N-(1-(2-(1,1-difluoroethyl)-6-((1,1- | |
| | difluorospiro[2.3 ]hexan-5- | |
| | yl)oxy)pyrimidin-4-yl)-1H-pyrazolo[4,3- | |
| | c]pyridin-6-yl)acetamide | |
| 704 | 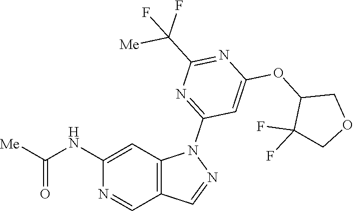 [SMILES: CC(=O)Nc1cc2c(cn1)cnn2-c1cc(OC2COCC2(F)F)nc(C(C)(F)F)n1] | LCMS m/z = 414.2 [M + H]⁺ 1H NMR (600 MHz, DMSO-d₆) (ppm) 10.76 (s, 1H), 9.41 (s, 1H), 8.97 (s, 1H), 8.67 (s, 1H), 7.42 (s, 1H), 5.86-5.73 (m, 1H), 4.43 (dd, J = 6.0, 11.1 Hz, 1H), 4.16-3.96 (m, 3H), 2.23 (t, J = 19.1 Hz, 3H), 2.16 (s, 3H) |
| | N-(1-(2-(1,1-difluoroethyl)-6-((4,4- | |
| | difluorotetrahydrofuran-3- | |
| | yl)oxy)pyrimidin-4-y1)-1H-pyrazolo[4,3- | |
| | c]pyridin-6-yl)acetamide | |
| 705 | 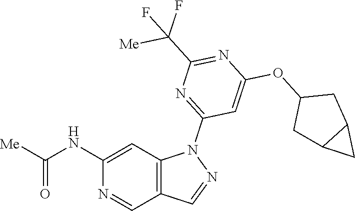 [SMILES: CC(=O)Nc1cc2c(cn1)cnn2-c1cc(OC2CC3CC3C2)nc(C(C)(F)F)n1] | LCMS m/z = 415.2 [M + H]⁺ 1H NMR (600 MHz, DMSO-d₆) (ppm) 10.74 (s, 1H), 9.39 (s, 1H), 8.95 (s, 1H), 8.63 (s, 1H), 7.21- 7.18 (m, 1H), 5.61 (t, J = 6.7 Hz, 1H), 2.36-2.27 (m, 2H), 2.25-2.17 (m, 3H), 2.15 (s, 3H), 1.93 (d, J = 14.9 Hz, 3H), 1.41-1.32 (m, 2H), 0.50 (s, 2H) |
| | N-(1-(6-(bicyclo[3.1.0]hexan-3-yloxy)-2- | |
| | (1,1-difluoroethyl)pyrimidin-4-yl)-1H- | |
| | pyrazolo[4,3-c]pyridin-6-yl)acetamide | |
| 706 | 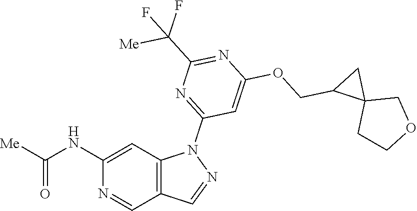 [SMILES: CC(=O)Nc1cc2c(cn1)cnn2-c1cc(OCC2CC23CCOC3)nc(C(C)(F)F)n1] | LCMS m/z = 445.2 [M + H]⁺ 1H NMR (600 MHz, DMSO-d₆) (ppm) 11.53 (s, 1H), 10.17 (s, 1H), 9.74 (s, 1H), 9.57-9.24 (m, 1H), 8.11-8.03 (m, 1H), 5.33 (br d, J = 6.9 Hz, 2H), 5.14-4.93 (m, 2H), 4.65-4.44 (m, 4H), 4.38 (br d, J = 8.4 Hz, 2H), 3.01 (t, J = 19.3 Hz, 3H), 2.96-2.85 (m, 5H), 2.84-2.73 (m, 1H), 2.73-2.50 (m, 3H), 2.41- 2.16 (m, 2H), 2.07-1.88 (m, 2H), 1.78-1.62 (m, 2H), 1.56-1.42 (m, 2H) |
| | N-(1-(6-((5-oxaspiro[2.4]heptan-1- | |
| | yl)methoxy)-2-(1,1- | |
| | difluoroethyl)pyrimidin-4-yl)-1H- | |
| | pyrazolo[4,3-c]pyridin-6-yl)acetamide | |
| 707 | 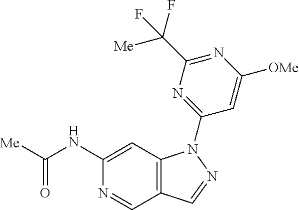 [SMILES: COc1cc(-n2ncc3cnc(NC(C)=O)cc32)nc(C(C)(F)F)n1] | LCMS m/z = 349.1 [M + H]⁺ 1H NMR (500 MHz, DMSO-d₆) (ppm) 10.79 (s, 1H), 9.47-9.30 (m, 1H), 9.02-8.90 (m, 1H), 8.70-8.62 (m, 1H), 7.35-7.26 (m, 1H), 4.06 (s, 3H), 2.25 (t, J = 19.2 Hz, 3H), 2.16 (s, 3H) |
| | N-(1-(2-(1,1-difluoroethyl)-6- | |
| | methoxypyrimidin-4-yl)-1H-pyrazolo[4,3- | |
| | c]pyridin-6-y1)acetamide | |
| 708 | 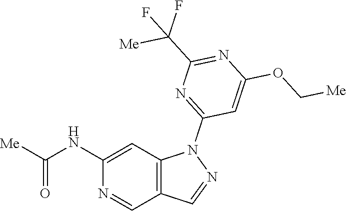 [SMILES: CCOc1cc(-n2ncc3cnc(NC(C)=O)cc32)nc(C(C)(F)F)n1] | LCMS m/z = 363.1 [M + H]⁺ 1H NMR (500 MHz, DMSO-d₆) (ppm) 10.79 (s, 1H), 9.41 (s, 1H), 8.97 (s, 1H), 8.66 (s, 1H), 7.28 (s, 1H), 4.57-4.42 (m, 2H), 2.24 (t, J = 19.2 Hz, 3H), 2.16 (s, 3H), 1.45- 1.34 (m, 3H) |
| | N-(1-(2-(1,1-difluoroethyl)-6- | |
| | ethoxypyrimidin-4-y1)-1H-pyrazolo[4,3- | |
| | c]pyridin-6-yl)acetamide | |
| 709 | 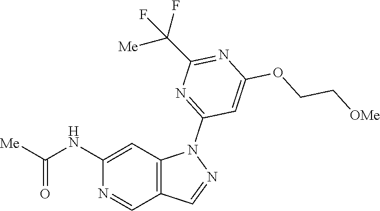 [SMILES: COCCOc1cc(-n2ncc3cnc(NC(C)=O)cc32)nc(C(C)(F)F)n1] | LCMS m/z = 393.1 [M + H]⁺ 1H NMR (500 MHz, DMSO-d₆) (ppm) 10.79 (s, 1H), 9.45-9.37 (m, 1H), 9.03-8.93 (m, 1H), 8.69-8.62 (m, 1H), 7.34-7.24 (m, 1H), 4.64- 4.53 (m, 2H), 2.55 (s, 3H), 2.24 (s, 3H), 2.16 (s, 3H) |
| | N-(1-(2-(1,1-difluoroethyl)-6-(2- | |
| | methoxyethoxy)pyrimidin-4-yl)-1H- | |
| | pyrazolo[4,3-c]pyridin-6-yl)acetamide | |
| 710 | 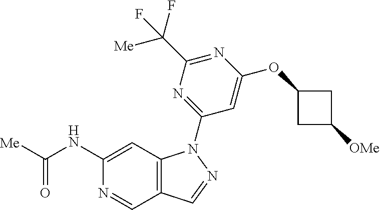 [SMILES: CO[C@H]1C[C@@H](Oc2cc(-n3ncc4cnc(NC(C)=O)cc43)nc(C(C)(F)F)n2)C1] | LCMS m/z = 419.2 [M + H]⁺ 1H NMR (500 MHz, DMSO-d₆) (ppm) 10.79 (s, 1H), 9.41 (s, 1H), 8.97 (s, 1H), 8.66 (s, 1H), 7.28 (s, 1H), 4.98 (quin, J = 7.3 Hz, 1H), 2.90 (br d, J = 3.1 Hz, 1H), 2.55 (s, 3H), 2.23 (s, 3H), 2.16 (s, 3H), 2.08-1.97 (m, 2H), 1.30-1.12 (m, 2H) |
| | N-(1-(2-(1,1-difluoroethyl)-6-((1s,3s)-3- | |
| | methoxycyclobutoxy)pyrimidin-4-yl)-1H- | |
| | pyrazolo[4,3-c]pyridin-6-yl)acetamide | |
| 711 | 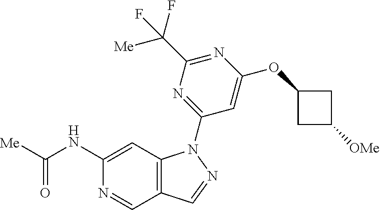 [SMILES: CO[C@H]1C[C@H](Oc2cc(-n3ncc4cnc(NC(C)=O)cc43)nc(C(C)(F)F)n2)C1] | LCMS m/z = 419.2 [M + H]⁺ 1H NMR (500 MHz, DMSO-d₆) (ppm) 10.79 (s, 1H), 9.41 (s, 1H), 8.97 (s, 1H), 8.66 (s, 1H), 7.26 (s, 1H), 5.48-5.30 (m, 1H), 5.03-4.89 (m, 1H), 4.18-4.03 (m, 1H), 2.44 (ddd, J = 4.3, 6.9, 17.5 Hz, 3H), 2.28-2.18 (m, 3H), 2.16 (s, 3H), 1.35-1.10 (m, 2H) |
| | N-(1-(2-(1,1-difluoroethyl)-6-((1r,3r)-3- | |
| | methoxycyclobutoxy)pyrimidin-4-yl)-1H- | |
| | pyrazolo[4,3-c]pyridin-6-yl)acetamide | |
| 712 | 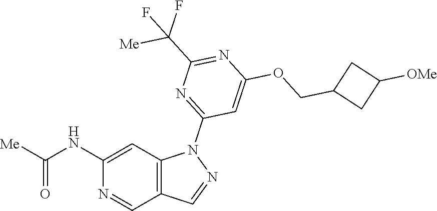 [SMILES: COC1CC(COc2cc(-n3ncc4cnc(NC(C)=O)cc43)nc(C(C)(F)F)n2)C1] | LCMS m/z = 433.2 [M + H]⁺ |
| | N-(1-(2-(1,1-difluoroethyl)-6-((3- | |
| | methoxycyclobutyl)methoxy)pyrimidin-4- | |
| | y1)-1H-pyrazolo[4,3-c]pyridin-6- | |
| | yl)acetamide | |
| 713 | 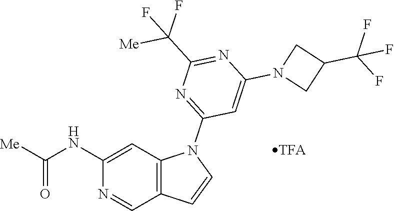 [SMILES: CC(=O)Nc1cc2c(ccn2-c2cc(N3CC(C(F)(F)F)C3)nc(C(C)(F)F)n2)cn1] | LCMS m/z = 440.1 [M + H]⁺ |
| | N-(1-(2-(1,1-difluoroethyl)-6-(3- | |
| | (trifluoromethyl)azetidin-1-yl)pyrimidin- | |
| | 4-y1)-1H-pyrrolo[3,2-c]pyridin-6- | |
| | yl)acetamide trifluoroacetate | |
| 714 | 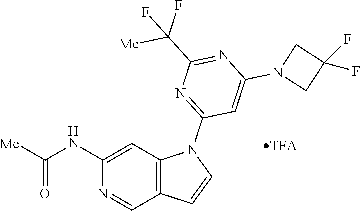 [SMILES: CC(=O)Nc1cc2c(ccn2-c2cc(N3CC(F)(F)C3)nc(C(C)(F)F)n2)cn1] | LCMS m/z = 408.1 [M + H]⁺ |
| | N-(1-(6-(3,3-difluoroazetidin-1-y1)-2-(1,1- | |
| | difluoroethyl)pyrimidin-4-y1)-1H- | |
| | pyrrolo[3,2-c]pyridin-6-yl)acetamide | |
| | trifluoroacetate | |
| 715 | 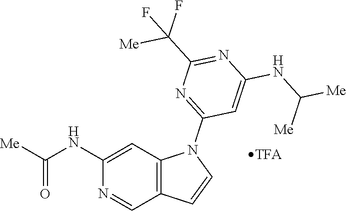 [SMILES: CC(=O)Nc1cc2c(ccn2-c2cc(NC(C)C)nc(C(C)(F)F)n2)cn1] | LCMS m/z = 374.2 [M + H]⁺ |
| | N-(1-(2-(1,1-difluoroethyl)-6- | |
| | (isopropylamino)pyrimidin-4-yl)-1H- | |
| | pyrrolo[3,2-c]pyridin-6-yl)acetamide | |
| | trifluoroacetate | |
| 716 | 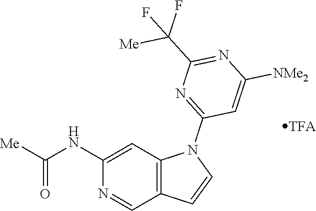 [SMILES: CC(=O)Nc1cc2c(ccn2-c2cc(N(C)C)nc(C(C)(F)F)n2)cn1] | LCMS m/z = 360.2 [M + H]⁺ |
| | N-(1-(2-(1,1-difluoroethyl)-6- | |
| | (dimethylamino)pyrimidin-4-yl)-1H- | |
| | pyrrolo[3,2-c]pyridin-6-yl)acetamide | |
| | trifluoroacetate | |
| 717 | 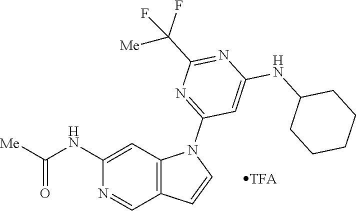 [SMILES: CC(=O)Nc1cc2c(ccn2-c2cc(NC3CCCCC3)nc(C(C)(F)F)n2)cn1] | LCMS m/z = 414.2 [M + H]⁺ |
| | N-(1-(6-(cyclohexylamino)-2-(1,1- | |
| | difluoroethyl)pyrimidin-4-yl)-1H- | |
| | pyrrolo[3,2-c]pyridin-6-y1)acetamide | |
| | trifluoroacetate | |
| 718 | 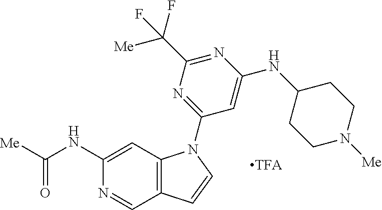 [SMILES: CC(=O)Nc1cc2c(ccn2-c2cc(NC3CCN(C)CC3)nc(C(C)(F)F)n2)cn1] | LCMS m/z = 429.2 [M + H]⁺ |
| | N-(1-(2-(1,1-difluoroethyl)-6-((1- | |
| | methylpiperidin-4-yl)amino)pyrimidin-4- | |
| | yl)-1H-pyrrolo[3,2-c]pyridin-6- | |
| | yl)acetamide trifluoroacetate | |
| 719 | 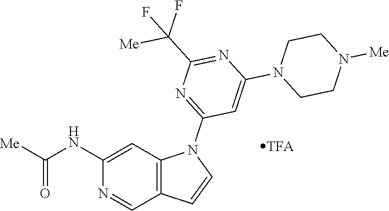 [SMILES: CC(=O)Nc1cc2c(ccn2-c2cc(N3CCN(C)CC3)nc(C(C)(F)F)n2)cn1] | LCMS m/z = 415.2 [M + H]⁺ |
| | N-(1-(2-(1,1-difluoroethyl)-6-(4- | |
| | methylpiperazin-1-yl)pyrimidin-4-yl)-1H- | |
| | pyrrolo[3,2-c]pyridin-6-yl)acetamide | |
| | trifluoroacetate | |
| 720 | 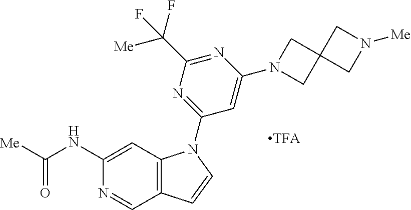 [SMILES: CC(=O)Nc1cc2c(ccn2-c2cc(N3CC4(CN(C)C4)C3)nc(C(C)(F)F)n2)cn1] | LCMS m/z = 427.2 [M + H]⁺ |
| | N-(1-(2-(1,1-difluoroethyl)-6-(6-methyl- | |
| | 2,6-diazaspiro[3.3]heptan-2-yl)pyrimidin- | |
| | 4-y1)-1H-pyrrolo[3,2-c]pyridin-6- | |
| | yl)acetamide trifluoroacetate | |
| 721 | 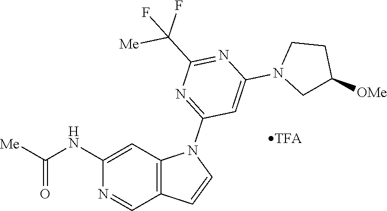 [SMILES: CO[C@@H]1CCN(c2cc(-n3ccc4cnc(NC(C)=O)cc43)nc(C(C)(F)F)n2)C1] | LCMS m/z = 416.2 [M + H]⁺ |
| | (R)-N-(1-(2-(1, 1-difluoroethyl)-6-(3- | |
| | methoxypyrrolidin-1-yl)pyrimidin-4-yl)- | |
| | 1H-pyrrolo[3,2-c]pyridin-6-yl)acetamide | |
| | trifluoroacetate | |
| 722 | 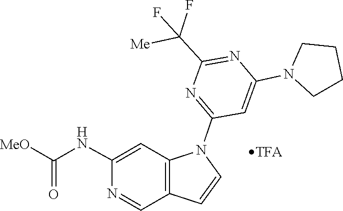 [SMILES: COC(=O)Nc1cc2c(ccn2-c2cc(N3CCCC3)nc(C(C)(F)F)n2)cn1] | LCMS m/z = 386.2 [M + H]⁺ |
| | N-(1-(2-(1,1-difluoroethyl)-6-(pyrrolidin- | |
| | 1-yl)pyrimidin-4-yl)-1H-pyrrolo[3,2- | |
| | c]pyridin-6-yl)acetamide trifluoroacetate | |
| 723 | 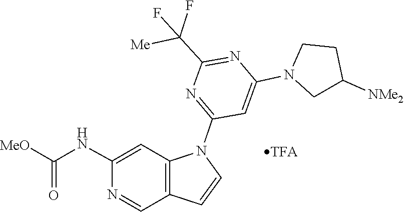 [SMILES: COC(=O)Nc1cc2c(ccn2-c2cc(N3CCC(N(C)C)C3)nc(C(C)(F)F)n2)cn1] | LCMS m/z = 429.2 [M + H]⁺ |
| | N-(1-(2-(1,1-difluoroethyl)-6-(3- | |
| | (dimethylamino)pyrrolidin-1- | |
| | yl)pyrimidin-4-yl)-1H-pyrrolo[3,2- | |
| | c]pyridin-6-yl)acetamide trifluoroacetate | |
| 724 | 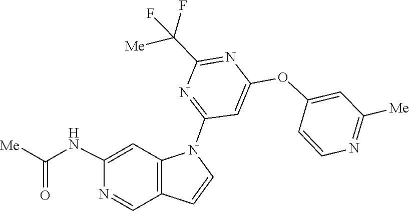 [SMILES: CC(=O)Nc1cc2c(ccn2-c2cc(Oc3ccnc(C)c3)nc(C(C)(F)F)n2)cn1] | LCMS m/z = 424.1 [M + H]⁺ |
| | N-(1-(2-(1,1-difluoroethyl)-6-((2- | |
| | methylpyridin-4-yl)oxy)pyrimidin-4-yl)- | |
| | 1H-pyrrolo[3,2-c]pyridin-6-yl)acetamide | |
| 725 | 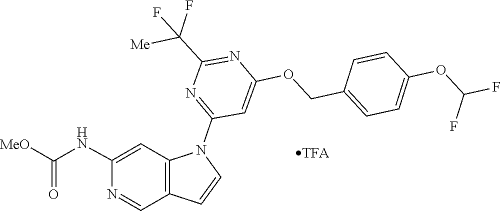 [SMILES: COC(=O)Nc1cc2c(ccn2-c2cc(OCc3ccc(OC(F)F)cc3)nc(C(C)(F)F)n2)cn1] | LCMS m/z = 489.1 [M + H]⁺ |
| | N-(1-(2-(1,1-difluoroethyl)-6-((4- | |
| | (difluoromethoxy)benzyl)oxy)pyrimidin- | |
| | 4-y1)-1H-pyrrolo[3,2-c]pyridin-6- | |
| | yl)acetamide trifluoroacetate | |
| 726 | 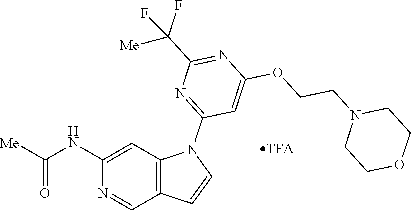 [SMILES: CC(=O)Nc1cc2c(ccn2-c2cc(OCCN3CCOCC3)nc(C(C)(F)F)n2)cn1] | LCMS m/z = 446.2 [M + H]⁺ |
| | N-(1-(2-(1,1-difluoroethyl)-6-(2- | |
| | morpholinoethoxy)pyrimidin-4-yl)-1H- | |
| | pyrrolo[3,2-c]pyridin-6-yl)acetamide | |
| | trifluoroacetate | |
| 727 | 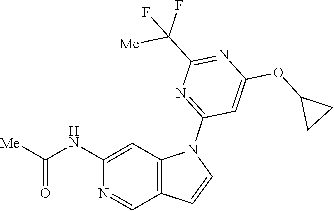 [SMILES: CC(=O)Nc1cc2c(ccn2-c2cc(OC3CC3)nc(C(C)(F)F)n2)cn1] | LCMS m/z = 373.1 [M + H]⁺ |
| | N-(1-(6-cyclopropoxy-2-(1,1- | |
| | difluoroethyl)pyrimidin-4-y1)-1H- | |
| | pyrrolo[3,2-c]pyridin-6-yl)acetamide | |
| 728 | 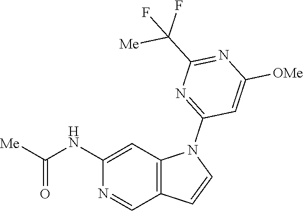 [SMILES: COc1cc(-n2ccc3cnc(NC(C)=O)cc32)nc(C(C)(F)F)n1] | LCMS m/z = 347.1 [M + H]⁺ |
| | N-(1-(2-(1,1-difluoroethyl)-6- | |
| | methoxypyrimidin-4-yl)-1H-pyrrolo[3,2- | |
| | c]pyridin-6-yl)acetamide | |
| 729 | 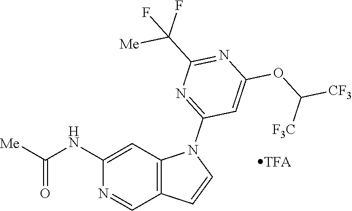 [SMILES: CC(=O)Nc1cc2c(ccn2-c2cc(OC(C(F)(F)F)C(F)(F)F)nc(C(C)(F)F)n2)cn1] | LCMS m/z = 483.1 [M + H]⁺ |
| | N-(1-(2-(1,1-difluoroethyl)-6- | |
| | ((1,1,1,3,3,3-hexafluoropropan-2- | |
| | yl)oxy)pyrimidin-4-yl)-1H-pyrrolo[3,2- | |
| | c]pyridin-6-yl)acetamide trifluoroacetate | |
| 730 | 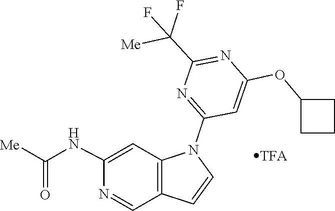 [SMILES: CC(=O)Nc1cc2c(ccn2-c2cc(OC3CCC3)nc(C(C)(F)F)n2)cn1] | LCMS m/z = 387.2 [M + H]⁺ |
| | N-(1-(6-cyclobutoxy-2-(1,1- | |
| | difluoroethyl)pyrimidin-4-y1)-1H- | |
| | pyrrolo[3,2-c]pyridin-6-yl)acetamide | |
| | trifluoroacetate | |
| 731 | 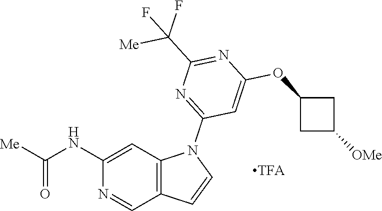 [SMILES: CO[C@H]1C[C@H](Oc2cc(-n3ccc4cnc(NC(C)=O)cc43)nc(C(C)(F)F)n2)C1] | LCMS m/z = 417.2 [M + H]⁺ |
| | N-(1-(2-(1,1-difluoroethyl)-6-((1r,3r)-3- | |
| | methoxycyclobutoxy)pyrimidin-4-yl)-1H- | |
| | pyrrolo[3,2-c]pyridin-6-yl)acetamide | |
| 732 | 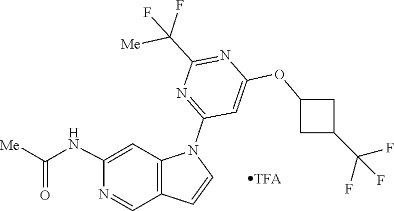 [SMILES: CC(=O)Nc1cc2c(ccn2-c2cc(OC3CC(C(F)(F)F)C3)nc(C(C)(F)F)n2)cn1] | LCMS m/z = 455.1 [M + H]⁺ |
| | N-(1-(2-(1,1-difluoroethyl)-6-(3- | |
| | (trifluoromethyl)cyclobutoxy)pyrimidin- | |
| | 4-y1)-1H-pyrrolo[3,2-c]pyridin-6- | |
| | yl)acetamide trifluoroacetate | |
| 733 | 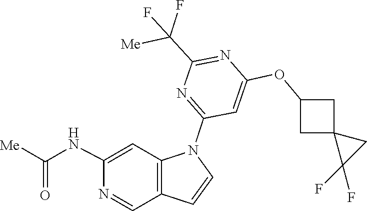 [SMILES: CC(=O)Nc1cc2c(ccn2-c2cc(OC3CC4(C3)CC4(F)F)nc(C(C)(F)F)n2)cn1] | LCMS m/z = 449.1 [M + H]⁺ |
| | N-(1-(2-(1,1-difluoroethyl)-6-((1,1- | |
| | difluorospiro[2.3 ]hexan-5- | |
| | yl)oxy)pyrimidin-4-yl)-1H-pyrrolo[3,2- | |
| | c]pyridin-6-yl)acetamide | |
| 734 | 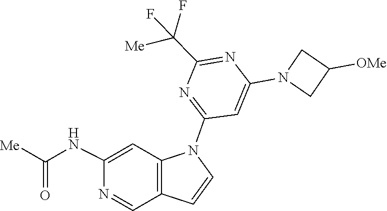 [SMILES: COC1CN(c2cc(-n3ccc4cnc(NC(C)=O)cc43)nc(C(C)(F)F)n2)C1] | LCMS m/z = 402.2 [M + H]⁺ |
| | N-(1-(2-(1,1-difluoroethyl)-6-(3- | |
| | methoxyazetidin-1-yl)pyrimidin-4-yl)-1H- | |
| | pyrrolo[3,2-c]pyridin-6-yl)acetamide | |
| 735 | 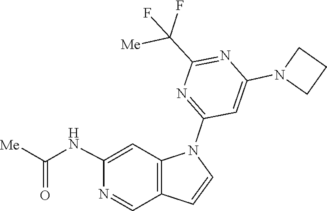 [SMILES: CC(=O)Nc1cc2c(ccn2-c2cc(N3CCC3)nc(C(C)(F)F)n2)cn1] | LCMS m/z = 372.2 [M + H]⁺ |
| | N-(1-(6-(azetidin-1-y1)-2-(1,1- | |
| | difluoroethyl)pyrimidin-4-y1)-1H- | |
| | pyrrolo[3,2-c]pyridin-6-yl)acetamide | |
| 736 | 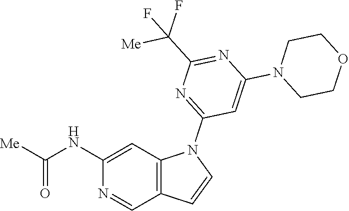 [SMILES: CC(=O)Nc1cc2c(ccn2-c2cc(N3CCOCC3)nc(C(C)(F)F)n2)cn1] | LCMS m/z = 402.2 [M + H]⁺ |
| | N-(1-(2-(1,1-difluoroethyl)-6- | |
| | morpholinopyrimidin-4-y1)-1H- | |
| | pyrrolo[3,2-c]pyridin-6-y1)acetamide | |
| 737 | 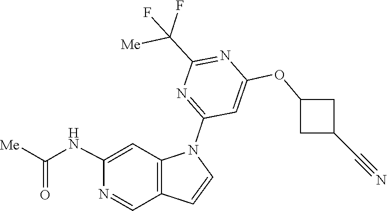 [SMILES: CC(=O)Nc1cc2c(ccn2-c2cc(OC3CC(C#N)C3)nc(C(C)(F)F)n2)cn1] | LCMS m/z = 412.1 [M + H]⁺ |
| | N-(1-(6-(3-cyanocyclobutoxy)-2-(1,1- | |
| | difluoroethyl)pyrimidin-4-yl)-1H- | |
| | pyrrolo[3,2-c]pyridin-6-yl)acetamide | |
| 738 | 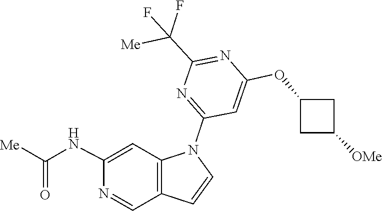 [SMILES: CO[C@H]1C[C@@H](Oc2cc(-n3ccc4cnc(NC(C)=O)cc43)nc(C(C)(F)F)n2)C1] | LCMS m/z = 417.2 [M + H]⁺ |
| | N-(1-(2-(1,1-difluoroethyl)-6-((1s,3s)-3- | |
| | methoxycyclobutoxy)pyrimidin-4-yl)-1H- | |
| | pyrrolo[3,2-c]pyridin-6-yl)acetamide | |
| 739 | 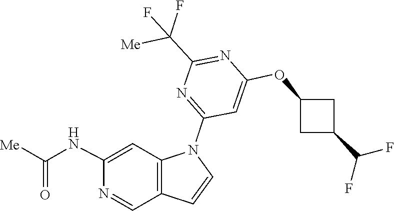 [SMILES: CC(=O)Nc1cc2c(ccn2-c2cc(O[C@H]3C[C@@H](C(F)F)C3)nc(C(C)(F)F)n2)cn1] | LCMS m/z = 437.1 [M + H]⁺ |
| | N-(1-(2-(1,1-difluoroethyl)-6-((1s,3s)-3- | |
| | (difluoromethyl)cyclobutoxy)pyrimidin-4- | |
| | y1)-1H-pyrrolo[3,2-c]pyridin-6- | |
| | yl)acetamide | |
| 740 | 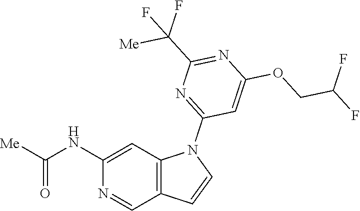 [SMILES: CC(=O)Nc1cc2c(ccn2-c2cc(OCC(F)F)nc(C(C)(F)F)n2)cn1] | LCMS m/z = 397.1 [M + H]⁺ |
| | N-(1-(6-(2,2-difluoroethoxy)-2-(1,1- | |
| | difluoroethyl)pyrimidin-4-yl)-1H- | |
| | pyrrolo[3,2-c]pyridin-6-yl)acetamide | |
| 741 | 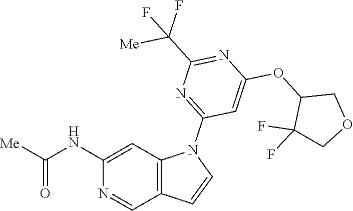 [SMILES: CC(=O)Nc1cc2c(ccn2-c2cc(OC3COCC3(F)F)nc(C(C)(F)F)n2)cn1] | LCMS m/z = 439.1 [M + H]⁺ |
| | N-(1-(2-(1,1-difluoroethyl)-6-((4,4- | |
| | difluorotetrahydrofuran-3- | |
| | yl)oxy)pyrimidin-4-y1)-1H-pyrrolo[3,2- | |
| | c]pyridin-6-yl)acetamide | |
| 742 | 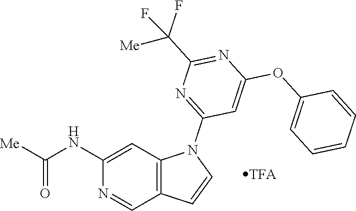 [SMILES: CC(=O)Nc1cc2c(ccn2-c2cc(Oc3ccccc3)nc(C(C)(F)F)n2)cn1] | LCMS m/z = 409.1 [M + H]⁺ |
| | N-(1-(2-(1,1-difluoroethyl)-6- | |
| | phenoxypyrimidin-4-yl)-1H-pyrrolo[3,2- | |
| | c]pyridin-6-yl)acetamide trifluoroacetate | |
| 743 | 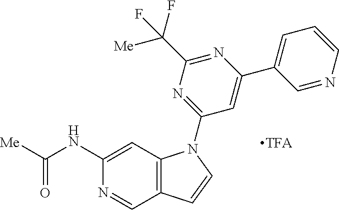 [SMILES: CC(=O)Nc1cc2c(ccn2-c2cc(-c3cccnc3)nc(C(C)(F)F)n2)cn1] | LCMS m/z = 394.1 [M + H]⁺ |
| | N-(1-(2-(1,1-difluoroethyl)-6-(pyridin-3- | |
| | yl)pyrimidin-4-yl)-1H-pyrrolo[3,2- | |
| | c]pyridin-6-yl)acetamide trifluoroacetate | |
| 744 | 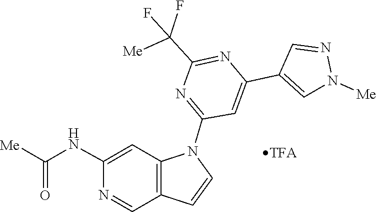 [SMILES: CC(=O)Nc1cc2c(ccn2-c2cc(-c3cnn(C)c3)nc(C(C)(F)F)n2)cn1] | LCMS m/z = 397.1 [M + H]⁺ |
| | N-(1-(2-(1,1-difluoroethyl)-6-(1-methyl- | |
| | 1H-pyrazol-4-yl)pyrimidin-4-y1)-1H- | |
| | pyrrolo[3,2-c]pyridin-6-yl)acetamide | |
| | trifluoroacetate | |
| 745 | 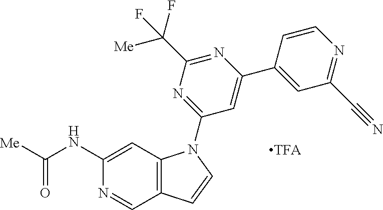 [SMILES: CC(=O)Nc1cc2c(ccn2-c2cc(-c3ccnc(C#N)c3)nc(C(C)(F)F)n2)cn1] | LCMS m/z = 419.1 [M + H]⁺ |
| | N-(1-(6-(2-cyanopyridin-4-yl)-2-(1,1- | |
| | difluoroethyl)pyrimidin-4-y1)-1H- | |
| | pyrrolo[3,2-c]pyridin-6-yl)acetamide | |
| | trifluoroacetate | |
| 746 | 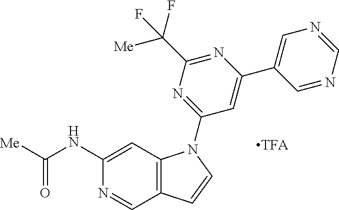 [SMILES: CC(=O)Nc1cc2c(ccn2-c2cc(-c3cncnc3)nc(C(C)(F)F)n2)cn1] | LCMS m/z = 395.1 [M + H]⁺ |
| | N-(1-(2-(1,1-difluoroethyl)-[4,5′- | |
| | bipyrimidin]-6-y1)-1H-pyrrolo[3,2- | |
| | c]pyridin-6-yl)acetamide trifluoroacetate | |
| 747 | 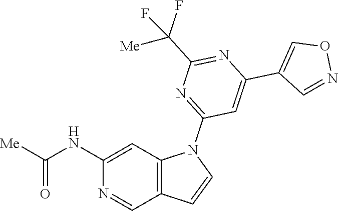 [SMILES: CC(=O)Nc1cc2c(ccn2-c2cc(-c3cnoc3)nc(C(C)(F)F)n2)cn1] | LCMS m/z = 384.1 [M + H]⁺ |
| | N-(1-(2-(1,1-difluoroethyl)-6-(isoxazol-4- | |
| | yl)pyrimidin-4-y1)-1H-pyrrolo[3,2- | |
| | c]pyridin-6-yl)acetamide | |
| 748 | 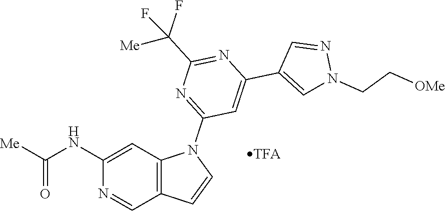 [SMILES: COCCn1cc(-c2cc(-n3ccc4cnc(NC(C)=O)cc43)nc(C(C)(F)F)n2)cn1] | LCMS m/z = 441.2 [M + H]⁺ |
| | N-(1-(2-(1,1-difluoroethyl)-6-(1-(2- | |
| | methoxyethyl)-1H-pyrazol-4- | |
| | yl)pyrimidin-4-yl)-1H-pyrrolo[3,2- | |
| | c]pyridin-6-yl)acetamide trifluoroacetate | |
| 749 | 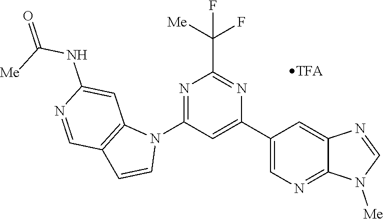 [SMILES: CC(=O)Nc1cc2c(ccn2-c2cc(-c3cnc4c(c3)ncn4C)nc(C(C)(F)F)n2)cn1] | LCMS m/z = 448.2 [M + H]+ |
| | N-(1-(2-(1,1-difluoroethyl)-6-(3-methyl- | |
| | 3H-imidazo[4,5-b]pyridin-6-yl)pyrimidin- | |
| | 4-y1)-1H-pyrrolo[3,2-c]pyridin-6- | |
| | yl)acetamide trifluoroacetate | |
| 750 | 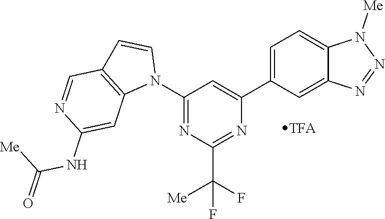 [SMILES: CC(=O)Nc1cc2c(ccn2-c2cc(-c3ccc4c(c3)nnn4C)nc(C(C)(F)F)n2)cn1] | LCMS m/z = 448.2 [M + H]⁺ |
| | N-(1-(2-(1,1-difluoroethyl)-6-(1-methyl- | |
| | 1H-benzo[d][1,2,3]triazol-5-yl)pyrimidin- | |
| | 4-yl)-1H-pyrrolo[3,2-c]pyridin-6- | |
| | yl)acetamide trifluoroacetate | |
| 751 | 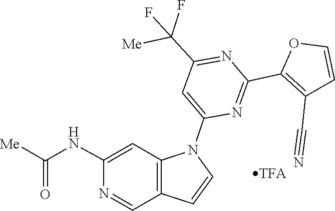 [SMILES: CC(=O)Nc1cc2c(ccn2-c2cc(C(C)(F)F)nc(-c3occc3C#N)n2)cn1] | LCMS m/z = 408.1 [M + H]⁺ |
| | N-(1-(6-(3-cyanofuran-2-y1)-2-(1,1- | |
| | difluoroethyl)pyrimidin-4-yl)-1H- | |
| | pyrrolo[3,2-c]pyridin-6-yl)acetamide | |
| | trifluoroacetate | |
| 752 | 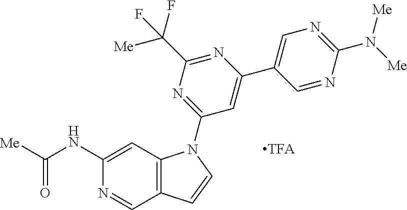 [SMILES: CC(=O)Nc1cc2c(ccn2-c2cc(-c3cnc(N(C)C)nc3)nc(C(C)(F)F)n2)cn1] | LCMS m/z = 438.2 [M + H]⁺ |
| | N-(1-(2-(1,1-difluoroethyl)-2′- | |
| | (dimethylamino)-[4,5′-bipyrimidin]-6-yl)- | |
| | 1H-pyrrolo[3,2-c]pyridin-6-yl)acetamide | |
| | trifluoroacetate | |
| 753 | 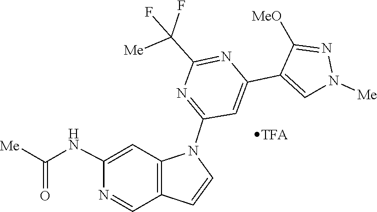 [SMILES: COc1nn(C)cc1-c1cc(-n2ccc3cnc(NC(C)=O)cc32)nc(C(C)(F)F)n1] | LCMS m/z = 427.2 [M + H]⁺ |
| | N-(1-(2-(1,1-difluoroethyl)-6-(3-methoxy- | |
| | 1-methyl-1H-pyrazol-4-yl)pyrimidin-4- | |
| | yl)-1H-pyrrolo[3,2-c]pyridin-6- | |
| | yl)acetamide trifluoroacetate | |
| 754 | 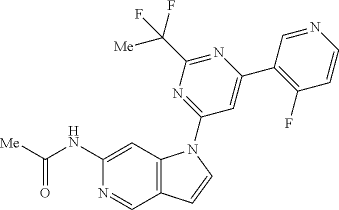 [SMILES: CC(=O)Nc1cc2c(ccn2-c2cc(-c3cnccc3F)nc(C(C)(F)F)n2)cn1] | LCMS m/z = 412.1 [M + H]⁺ |
| | N-(1-(2-(1,1-difluoroethyl)-6-(4- | |
| | fluoropyridin-3-yl)pyrimidin-4-y1)-1H- | |
| | pyrrolo[3,2-c]pyridin-6-yl)acetamide | |
| 755 | 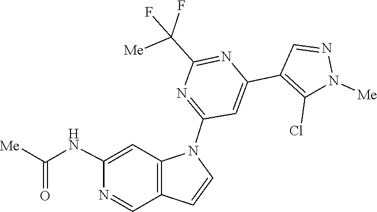 [SMILES: CC(=O)Nc1cc2c(ccn2-c2cc(-c3cnn(C)c3Cl)nc(C(C)(F)F)n2)cn1] | LCMS m/z = 431.1 [M + H]⁺ |
| | N-(1-(6-(5-chloro-1-methyl-1H-pyrazol-4- | |
| | yl)-2-(1,1-difluoroethyl)pyrimidin-4-yl)- | |
| | 1H-pyrrolo[3,2-c]pyridin-6-yl)acetamide | |
| 756 | 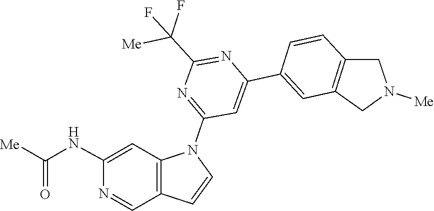 [SMILES: CC(=O)Nc1cc2c(ccn2-c2cc(-c3ccc4c(c3)CN(C)C4)nc(C(C)(F)F)n2)cn1] | LCMS m/z = 448.2 [M + H]⁺ |
| | N-(1-(2-(1,1-difluoroethyl)-6-(2- | |
| | methylisoindolin-5-yl)pyrimidin-4-yl)- | |
| | 1H-pyrrolo[3,2-c]pyridin-6-yl)acetamide | |
| 757 | 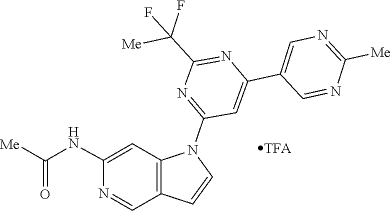 [SMILES: CC(=O)Nc1cc2c(ccn2-c2cc(-c3cnc(C)nc3)nc(C(C)(F)F)n2)cn1] | LCMS m/z = 409.1 [M + H]⁺ |
| | N-(1-(2-(1,1-difluoroethyl)-2′-methyl- | |
| | [4,5′-bipyrimidin]-6-y1)-1H-pyrrolo[3,2- | |
| | c]pyridin-6-yl)acetamide trifluoroacetate | |
| 758 | 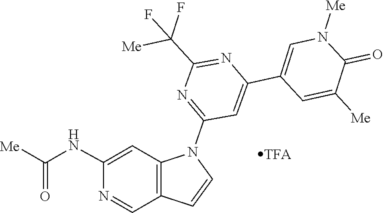 [SMILES: CC(=O)Nc1cc2c(ccn2-c2cc(-c3cc(C)c(=O)n(C)c3)nc(C(C)(F)F)n2)cn1] | LCMS m/z = 438.2 [M + H]⁺ |
| | N-(1-(2-(1,1-difluoroethyl)-6-(1,5- | |
| | dimethyl-6-oxo-1,6-dihydropyridin-3- | |
| | yl)pyrimidin-4-y1)-1H-pyrrolo[3,2- | |
| | c]pyridin-6-yl)acetamide trifluoroacetate | |
| 759 | 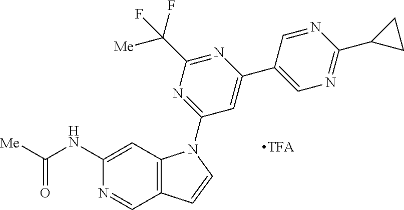 [SMILES: CC(=O)Nc1cc2c(ccn2-c2cc(-c3cnc(C4CC4)nc3)nc(C(C)(F)F)n2)cn1] | LCMS m/z = 435.2 [M + H]⁺ |
| | N-(1-(2′-cyclopropyl-2-(1,1- | |
| | difluoroethyl)-[4,5′-bipyrimidin]-6-yl)- | |
| | 1H-pyrrolo[3,2-c]pyridin-6-yl)acetamide | |
| 760 | 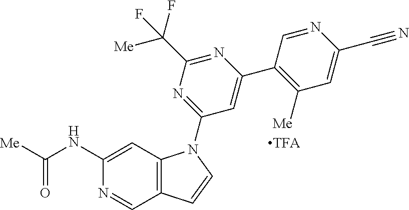 [SMILES: CC(=O)Nc1cc2c(ccn2-c2cc(-c3cnc(C#N)cc3C)nc(C(C)(F)F)n2)cn1] | LCMS m/z = 433.1 [M + H]⁺ |
| | N-(1-(6-(6-cyano-4-methylpyridin-3-yl)- | |
| | 2-(1,1-difluoroethyl)pyrimidin-4-yl)-1H- | |
| | pyrrolo[3,2-c]pyridin-6-yl)acetamide | |
| | trifluoroacetate | |
| 761 | 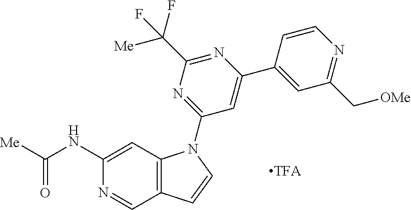 [SMILES: COCc1cc(-c2cc(-n3ccc4cnc(NC(C)=O)cc43)nc(C(C)(F)F)n2)ccn1] | LCMS m/z = 438.2 [M + H]⁺ |
| | N-(1-(2-(1,1-difluoroethyl)-6-(2- | |
| | (methoxymethyl)pyridin-4-yl)pyrimidin- | |
| | 4-yl)-1H-pyrrolo[3,2-c]pyridin-6- | |
| | yl)acetamide trifluoroacetate | |
| 762 | 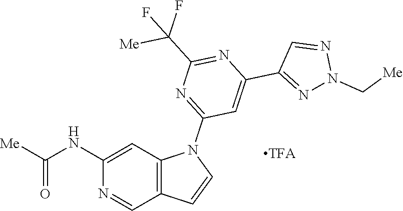 [SMILES: CCn1ncc(-c2cc(-n3ccc4cnc(NC(C)=O)cc43)nc(C(C)(F)F)n2)n1] | LCMS m/z = 412.2 [M + H]⁺ |
| | N-(1-(2-(1,1-difluoroethyl)-6-(2-ethyl-2H- | |
| | 1,2,3-triazol-4-yl)pyrimidin-4-yl)-1H- | |
| | pyrrolo[3,2-c]pyridin-6-yl)acetamide | |
| | trifluoroacetate | |
| 763 | 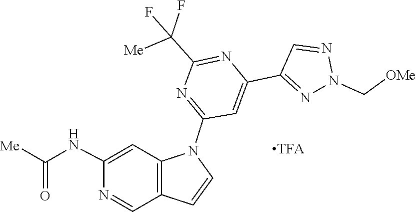 [SMILES: COCn1ncc(-c2cc(-n3ccc4cnc(NC(C)=O)cc43)nc(C(C)(F)F)n2)n1] | LCMS m/z = 428.2 [M + H]⁺ |
| | N-(1-(2-(1,1-difluoroethyl)-6-(2- | |
| | (methoxymethyl)-2H-1,2,3-triazol-4- | |
| | yl)pyrimidin-4-yl)-1H-pyrrolo[3,2- | |
| | c]pyridin-6-yl)acetamide trifluoroacetate | |
| 764 | 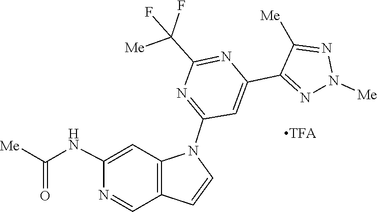 [SMILES: CC(=O)Nc1cc2c(ccn2-c2cc(-c3nn(C)nc3C)nc(C(C)(F)F)n2)cn1] | LCMS m/z = 412.2 [M + H]⁺ |
| | N-(1-(2-(1,1-difluoroethyl)-6-(2,5- | |
| | dimethyl-2H-1,2,3-triazol-4-yl)pyrimidin- | |
| | 4-y1)-1H-pyrrolo[3,2-c]pyridin-6- | |
| | y1)acetamide trifluoroacetate | |
| 765 | 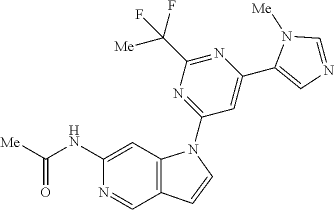 [SMILES: CC(=O)Nc1cc2c(ccn2-c2cc(-c3cncn3C)nc(C(C)(F)F)n2)cn1] | LCMS m/z = 397.1 [M + H]⁺ |
| | N-(1-(2-(1,1-difluoroethyl)-6-(1-methyl- | |
| | 1H-imidazol-5-yl)pyrimidin-4-yl)-1H- | |
| | pyrrolo[3,2-c]pyridin-6-yl)acetamide | |
| 766 | 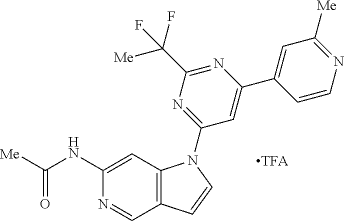 [SMILES: CC(=O)Nc1cc2c(ccn2-c2cc(-c3ccnc(C)c3)nc(C(C)(F)F)n2)cn1] | LCMS m/z = 408.2 [M + H]⁺ |
| | N-(1-(2-(1,1-difluoroethyl)-6-(2- | |
| | methylpyridin-4-yl)pyrimidin-4-yl)-1H- | |
| | pyrrolo[3,2-c]pyridin-6-yl)acetamide | |
| | trifluoroacetate | |
| 767 | 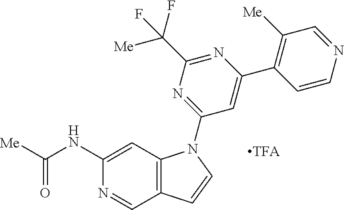 [SMILES: CC(=O)Nc1cc2c(ccn2-c2cc(-c3ccncc3C)nc(C(C)(F)F)n2)cn1] | LCMS m/z = 408.2 [M + H]⁺ |
| | N-(1-(2-(1,1-difluoroethyl)-6-(3- | |
| | methylpyridin-4-y1)pyrimidin-4-yl)-1H- | |
| | pyrrolo[3,2-c]pyridin-6-yl)acetamide | |
| | trifluoroacetate | |
| 768 | 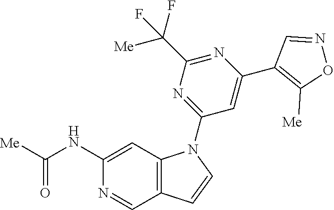 [SMILES: CC(=O)Nc1cc2c(ccn2-c2cc(-c3cnoc3C)nc(C(C)(F)F)n2)cn1] | LCMS m/z = 398.1 [M + H]⁺ |
| | N-(1-(2-(1,1-difluoroethyl)-6-(5- | |
| | methylisoxazol-4-yl)pyrimidin-4-yl)-1H- | |
| | pyrrolo[3,2-c]pyridin-6-y1)acetamide | |
| 769 | 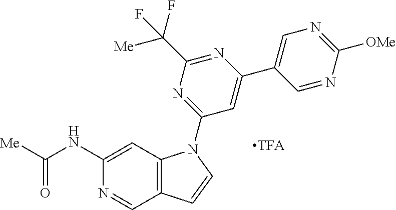 [SMILES: COc1ncc(-c2cc(-n3ccc4cnc(NC(C)=O)cc43)nc(C(C)(F)F)n2)cn1] | LCMS m/z = 425.1 [M + H]⁺ |
| | N-(1-(2-(1,1-difluoroethyl)-2′-methoxy- | |
| | [4,5′-bipyrimidin]-6-y1)-1H-pyrrolo[3,2- | |
| | c]pyridin-6-yl)acetamide trifluoroacetate | |
| 770 | 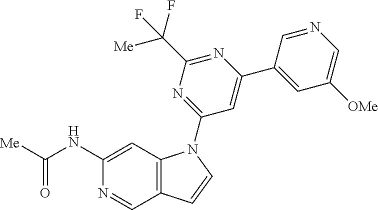 [SMILES: COc1cncc(-c2cc(-n3ccc4cnc(NC(C)=O)cc43)nc(C(C)(F)F)n2)c1] | LCMS m/z = 424.1 [M + H]⁺ |
| | N-(1-(2-(1,1-difluoroethyl)-6-(5- | |
| | methoxypyridin-3-y1)pyrimidin-4-y1)-1H- | |
| | pyrrolo[3,2-c]pyridin-6-yl)acetamide | |
| 771 | 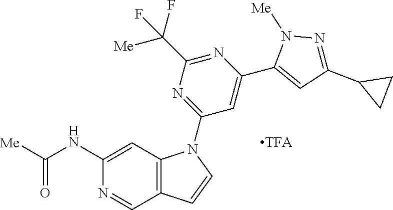 [SMILES: CC(=O)Nc1cc2c(ccn2-c2cc(-c3cc(C4CC4)nn3C)nc(C(C)(F)F)n2)cn1] | LCMS m/z = 437.2 [M + H]⁺ |
| | N-(1-(6-(3-cyclopropyl-1-methyl-1H- | |
| | pyrazol-5-yl)-2-(1,1- | |
| | difluoroethyl)pyrimidin-4-y1)-1H- | |
| | pyrrolo[3,2-c]pyridin-6-yl)acetamide | |
| | trifluoroacetate | |
| 772 | 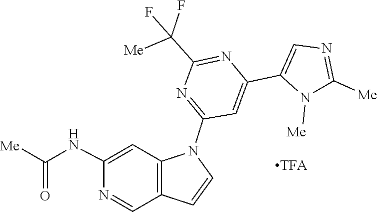 [SMILES: CC(=O)Nc1cc2c(ccn2-c2cc(-c3cnc(C)n3C)nc(C(C)(F)F)n2)cn1] | LCMS m/z = 411.2 [M + H]⁺ |
| | N-(1-(2-(1,1-difluoroethyl)-6-(1,2- | |
| | dimethyl-1H-imidazol-5-yl)pyrimidin-4- | |
| | y1)-1H-pyrrolo[3,2-c]pyridin-6- | |
| | yl)acetamide trifluoroacetate | |
| 773 | 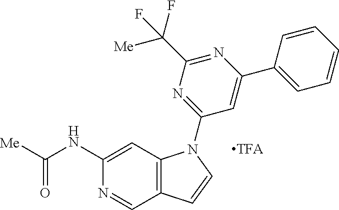 [SMILES: CC(=O)Nc1cc2c(ccn2-c2cc(-c3ccccc3)nc(C(C)(F)F)n2)cn1] | LCMS m/z = 393 [M + H]⁺ |
| | N-(1-(2-(1,1-difluoroethyl)-6- | |
| | phenylpyrimidin-4-yl)-1H-pyrrolo[3,2- | |
| | c]pyridin-6-yl)acetamide trifluoroacetate | |
| 774 | 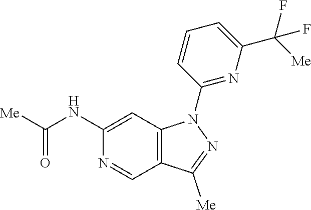 [SMILES: CC(=O)Nc1cc2c(cn1)c(C)nn2-c1cccc(C(C)(F)F)n1] | LCMS m/z = 332 [M + H]⁺ 1H NMR (500 MHz, DMSO-d₆) δ 10.74 (s, 1H), 9.35 (d, J = 0.76 Hz, 1H), 8.95 (d, J = 1.07 Hz, 1H), 8.13- 8.21 (m, 1H), 8.02-8.11 (m, 1H), 7.61 (dd, J = 0.76, 7.48 Hz, 1H), 2.65 (s, 3H), 2.27 (t, J = 19.45 Hz, 3H), 2.15 (s, 3H). |
| | N-(1-(6-(1,1-difluoroethyl)pyridin-2-yl)- | |
| | 3-methyl-1H-pyrazolo[4,3-c]pyridin-6- | |
| | yl)acetamide | |
| 775 | 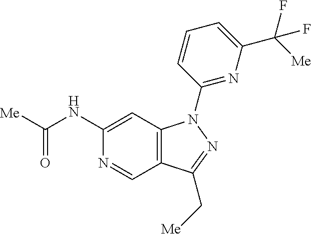 [SMILES: CCc1nn(-c2cccc(C(C)(F)F)n2)c2cc(NC(C)=O)ncc12] | LCMS m/z = 346 [M + H]⁺ 1H NMR (400 MHz, CDCl₃) δ 9.44 (s, 1H), 8.73 (s, 1H), 8.30 (br s, 1H), 8.09 (d, J = 8.28 Hz, 1H), 7.94 (t, J = 8.03 Hz, 1H), 7.55 (d, J = 7.53 Hz, 1H), 3.08 (q, J = 7.70 Hz, 2H), 2.26-2.41 (m, 6H), 1.49 (t, J = 7.65 Hz, 3H) |
| | N-(1-(6-(1,1-difluoroethyl)pyridin-2-yl)- | |
| | 3-ethyl-1H-pyrazolo[4,3-c]pyridin-6- | |
| | yl)acetamide | |
| 776 | 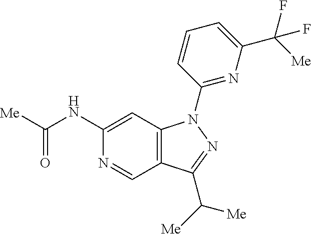 [SMILES: CC(=O)Nc1cc2c(cn1)c(C(C)C)nn2-c1cccc(C(C)(F)F)n1] | LCMS m/z = 360 [M + H]⁺ |
| | N-(1-(6-(1,1-difluoroethyl)pyridin-2-y1)- | |
| | 3-isopropyl-1H-pyrazolo[4,3-c]pyridin-6- | |
| | yl)acetamide | |
| 777 | 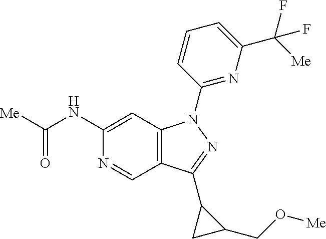 [SMILES: COCC1CC1c1nn(-c2cccc(C(C)(F)F)n2)c2cc(NC(C)=O)ncc12] | LCMS m/z = 402 [M + H]⁺ 1H NMR (400 MHz, CDCl₃) δ 9.43 (s, 1H), 8.84 (d, J = 0.75 Hz, 1H), 8.30 (br s, 1H), 8.02-8.09 (m, 1H), 7.94 (t, J = 8.03 Hz, 1H), 7.55 (d, J = 7.53 Hz, 1H), 3.43-3.49 (m, 1H), 3.25 (dd, J = 8.16, 10.42 Hz, 1H), 3.08 (s, 3H), 2.48 (dt, J = 6.02, 8.28 Hz, 1H), 2.26-2.39 (m, 6H), 1.74-1.80 (m, 1H), 1.35-1.44 (m, 2H) |
| | N-(1-(6-(1,1-difluoroethyl)pyridin-2-yl)- | |
| | 3-(2-(methoxymethyl)cyclopropyl)-1H- | |
| | pyrazolo[4,3-c]pyridin-6-yl)acetamide | |
| 778 | 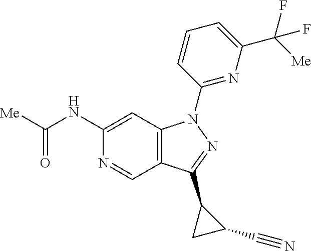 [SMILES: CC(=O)Nc1cc2c(cn1)c([C@@H]1C[C@H]1C#N)nn2-c1cccc(C(C)(F)F)n1] | LCMS m/z = 383 [M + H]⁺ 1H NMR (CDCl₃, 400 MHz) δ 9.52 (s, 1H), 8.85 (s, 1H), 8.60-8.80 (m, 1H), 7.90-8.00 (m, 2H), 7.60-7.65 (m, 1H), 2.98 (ddd, 1H, J = 4.5, 6.3, 9.0 Hz), 2.30-2.40 (m, 6H), 2.20 (ddd, 1H, J = 4.5, 6.0, 8.8 Hz), 1.80- 1.90 (m, 2H). |
| | or | |
| | 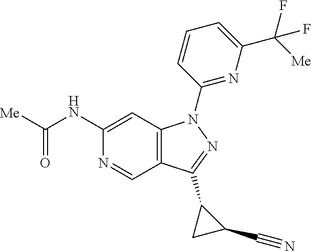 [SMILES: CC(=O)Nc1cc2c(cn1)c([C@H]1C[C@@H]1C#N)nn2-c1cccc(C(C)(F)F)n1] | |
| | N-(3-((1R,2R)-2-cyanocyclopropyl)-1-(6- | |
| | (1,1-difluoroethyl)pyridin-2-yl)-1H- | |
| | pyrazolo[4,3-c]pyridin-6-yl)acetamide or | |
| | N-(3-((1S,2S)-2-cyanocyclopropyl)-1-(6- | |
| | (1,1-difluoroethyl)pyridin-2-yl)-1H- | |
| | pyrazolo[4,3-c]pyridin-6-yl)acetamide | |
| 779 | 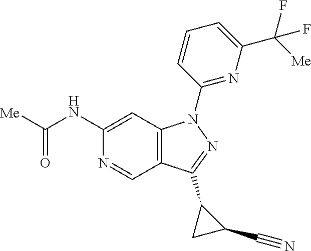 [SMILES: CC(=O)Nc1cc2c(cn1)c([C@H]1C[C@@H]1C#N)nn2-c1cccc(C(C)(F)F)n1] | LCMS m/z = 383 [M + H]⁺ 1H NMR (CDCl₃, 400 MHz) δ 9.47 (s, 1H), 8.84 (s, 1H), 8.32 (br s, 1H), 7.90-8.00 (m, 2H), 7.50-7.60 (m, 1H), 2.98 (ddd, 1H, J = 4.5, 6.3, 9.0 Hz), 2.30-2.40 (m, 6H), 2.19 (ddd, 1H, J = 4.5, 6.0, 8.8 Hz), 1.80-1.90 (m, 2H). |
| | or | |
| | 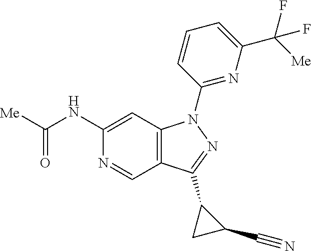 [SMILES: CC(=O)Nc1cc2c(cn1)c([C@H]1C[C@@H]1C#N)nn2-c1cccc(C(C)(F)F)n1] | |
| | N-(3-((1S,2S)-2-cyanocyclopropyl)-1-(6- | |
| | (1,1-difluoroethyl)pyridin-2-yl)-1H- | |
| | pyrazolo[4,3-c]pyridin-6-yl)acetamide or | |
| | N-(3-((1R,2R)-2-cyanocyclopropyl)-1-(6- | |
| | (1,1-difluoroethyl)pyridin-2-yl)-1H- | |
| | pyrazolo[4,3-c]pyridin-6-yl)acetamide | |
| 780 | 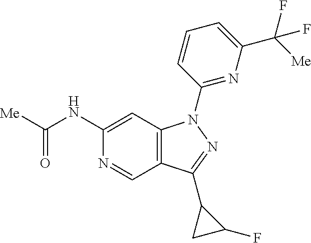 [SMILES: CC(=O)Nc1cc2c(cn1)c(C1CC1F)nn2-c1cccc(C(C)(F)F)n1] | LCMS m/z = 376 [M + H]⁺ 1H NMR (500 MHz, DMSO-d₆) δ 10.77 (s, 1H), 9.34 (s, 1H), 9.10 (d, J = 0.92 Hz, 1H), 8.12-8.20 (m, 1H), 8.03 (d, J = 8.39 Hz, 1H), 7.61 (d, J = 7.63 Hz, 1H), 5.09-5.32 (m, 1H), 3.06-3.20 (m, 1H), 2.25 (t, J = 19.38 Hz, 3H), 2.16 (s, 3H), 1.72-1.84 (m, 1H), 1.53-1.63 (m, 1H). |
| | N-(1-(6-(1,1-difluoroethyl)pyridin-2-yl)- | |
| | 3-(2-fluorocyclopropyl)-1H-pyrazolo[4,3- | |
| | c]pyridin-6-yl)acetamide | |
| 781 | 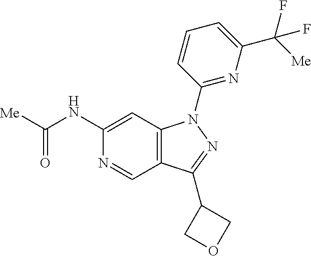 [SMILES: CC(=O)Nc1cc2c(cn1)c(C1COC1)nn2-c1cccc(C(C)(F)F)n1] | LCMS m/z = 374 [M + H]⁺ |
| | N-(1-(6-(1,1-difluoroethyl)pyridin-2-yl)- | |
| | 3-(oxetan-3-yl)-1H-pyrazolo[4,3- | |
| | c]pyridin-6-yl)acetamide | |
| 782 | 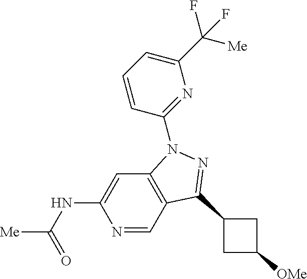 [SMILES: CO[C@H]1C[C@@H](c2nn(-c3cccc(C(C)(F)F)n3)c3cc(NC(C)=O)ncc32)C1] | LCMS m/z = 402 [M + H]⁺ |
| | N-(1-(6-(1,1-difluoroethyl)pyridin-2-yl)- | |
| | 3-((1s,3s)-3-methoxycyclobutyl)-1H- | |
| | pyrazolo[4,3-c]pyridin-6-yl)acetamide | |
| 783 | 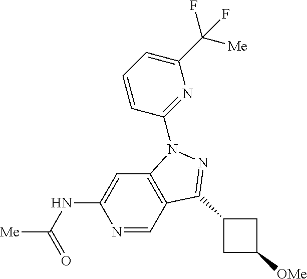 [SMILES: CO[C@H]1C[C@H](c2nn(-c3cccc(C(C)(F)F)n3)c3cc(NC(C)=O)ncc32)C1] | LCMS m/z = 402 [M + H]⁺ |
| | N-(1-(6-(1,1-difluoroethyl)pyridin-2-yl)- | |
| | 3-((1r,3r)-3-methoxycyclobutyl)-1H- | |
| | pyrazolo[4,3-c]pyridin-6-yl)acetamide | |
| 784 | 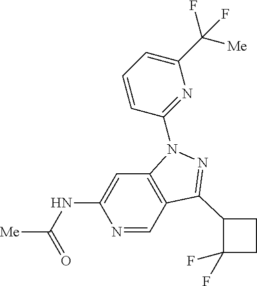 [SMILES: CC(=O)Nc1cc2c(cn1)c(C1CCC1(F)F)nn2-c1cccc(C(C)(F)F)n1] | LCMS m/z = 408 [M + H]⁺ |
| | N-(3-(2,2-difluorocyclobutyl)-1-(6-(1,1- | |
| | difluoroethyl)pyridin-2-y1)-1H- | |
| | pyrazolo[4,3-c]pyridin-6-yl)acetamide | |
| 785 | 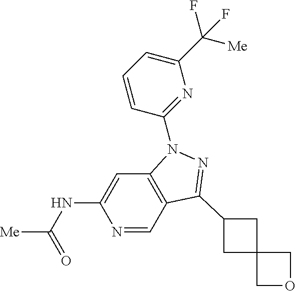 [SMILES: CC(=O)Nc1cc2c(cn1)c(C1CC3(COC3)C1)nn2-c1cccc(C(C)(F)F)n1] | LCMS m/z = 414 [M + H]⁺ 1H NMR (400 MHz, MeOD-d₄) δ 9.52 (s, 1H), 8.01-8.10 (m, 1H), 7.81- 7.93 (m, 2H), 7.62 (d, J = 7.53 Hz, 1H), 4.70 (s, 2H), 3.95 (t, J = 5.52 Hz, 1H), 3.74 (s, 2H), 2.64 (ddd, J = 2.76, 5.77, 6.78 Hz, 2H), 2.23 (s, 3H), 1.87-1.94 (m, 2H), 1.78 (t, J = 18.70 Hz, 3H) |
| | N-(1-(6-(1,1-difluoroethyl)pyridin-2-yl)- | |
| | 3-(2-oxaspiro[3.3]heptan-6-yl)-1H- | |
| | pyrazolo[4,3-c]pyridin-6-yl)acetamide | |
| 786 | 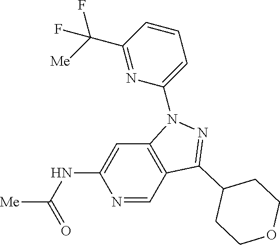 [SMILES: CC(=O)Nc1cc2c(cn1)c(C1CCOCC1)nn2-c1cccc(C(C)(F)F)n1] | LCMS m/z = 402 [M + H]⁺ 1H NMR (500 MHz, DMSO-d₆) δ10.76 (s, 1H), 9.36 (s, 1H), 9.07 (d, J = 1.07 Hz, 1H), 8.13-8.23 (m, 1H), 8.09 (d, J = 8.24 Hz, 1H), 7.54- 7.66 (m, 1H), 4.01 (td, J = 2.06, 9.46 Hz, 2H), 3.56 (dt, J = 2.21, 11.56 Hz, 2H), 2.26 (t, J = 19.45 Hz, 3H), 2.13-2.19 (m, 3H), 1.85- 2.08 (m, 5H). |
| | N-(1-(6-(1,1-difluoroethyl)pyridin-2-yl)- | |
| | 3-(tetrahydro-2H-pyran-4-yl)-1H- | |
| | pyrazolo[4,3-c]pyridin-6-yl)acetamide | |
| 787 | 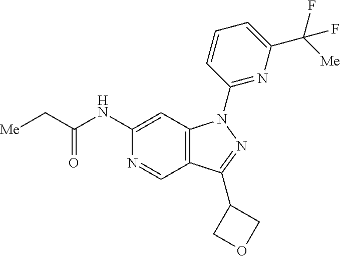 [SMILES: CCC(=O)Nc1cc2c(cn1)c(C1COC1)nn2-c1cccc(C(C)(F)F)n1] | LCMS m/z = 346 [M + H]⁺ 1H NMR (500 MHz, DMSO-d₆) δ 10.68 (s, 1H), 9.34 (d, J = 1.07 Hz, 1H), 8.95 (d, J = 1.07 Hz, 1H), 8.11- 8.23 (m, 1H), 8.07 (d, J = 7.93 Hz, 1H), 7.61 (dd, J = 0.69, 7.55 Hz, 1H), 2.65 (s, 3H), 2.45 (q, J = 7.63 Hz, 2H), 2.28 (t, J = 19.45 Hz, 3H), 1.11 (t, J = 7.55 Hz, 3H). |
| | N-(1-(6-(1,1-difluoroethyl)pyridin-2-yl)- | |
| | 3-methyl-1H-pyrazolo[4,3-c]pyridin-6- | |
| | yl)propionamide | |
| 788 | 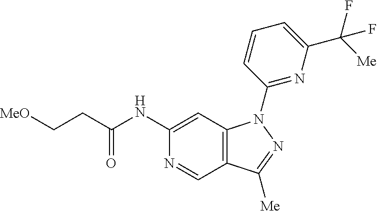 [SMILES: COCCC(=O)Nc1cc2c(cn1)c(C)nn2-c1cccc(C(C)(F)F)n1] | LCMS m/z = 376 [M + H]⁺ |
| | N-(1-(6-(1,1-difluoroethyl)pyridin-2-yl)- | |
| | 3-methyl-1H-pyrazolo[4,3-c]pyridin-6- | |
| | yl)-3-methoxypropanamide | |
| 789 | 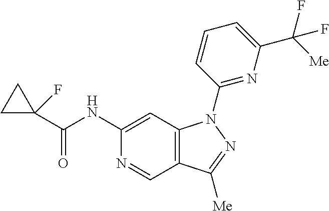 [SMILES: Cc1nn(-c2cccc(C(C)(F)F)n2)c2cc(NC(=O)C3(F)CC3)ncc12] | LCMS m/z = 376 [M + H]⁺ |
| | N-(1-(6-(1,1-difluoroethyl)pyridin-2-yl)- | |
| | 3-methyl-1H-pyrazolo[4,3-c]pyridin-6- | |
| | y1)-1-fluorocyclopropane-1-carboxamide | |
| 790 | 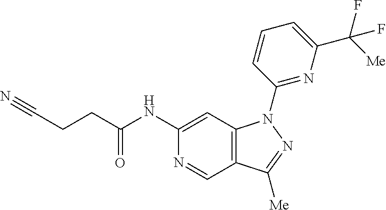 [SMILES: Cc1nn(-c2cccc(C(C)(F)F)n2)c2cc(NC(=O)CCC#N)ncc12] | LCMS m/z = 371 [M + H]⁺ 1H NMR (DMSO-d₆, 600 MHz) δ 10.86 (br s, 1H), 9.32 (br s, 1H), 8.96 (s, 1H), 8.17 (t, 1H, J = 8.0 Hz), 8.07 (d, 1H, J = 8.0 Hz), 7.62 (d, 1H, J = 8.0 Hz), 2.8-2.9 (m, 2H), 2.7-2.8 (m, 2H), 2.66 (s, 3H), 2.27 (t, 3H, J = 19.6 Hz) |
| | 3-cyano-N-(1-(6-(1,1- | |
| | difluoroethyl)pyridin-2-yl)-3-methyl-1H- | |
| | pyrazolo[4,3-c]pyridin-6-yl)propanamide | |
| 791 | 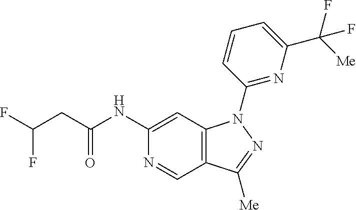 [SMILES: Cc1nn(-c2cccc(C(C)(F)F)n2)c2cc(NC(=O)CC(F)F)ncc12] | LCMS m/z = 382 [M + H]⁺ |
| | N-(1-(6-(1,1-difluoroethyl)pyridin-2-yl)- | |
| | 3-methyl-1H-pyrazolo[4,3-c]pyridin-6- | |
| | yl)-3,3-difluoropropanamide | |
| 792 | 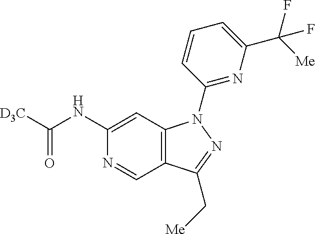 [SMILES: [2H]C([2H])([2H])C(=O)Nc1cc2c(cn1)c(CC)nn2-c1cccc(C(C)(F)F)n1] | LCMS m/z = 349 [M + H]⁺ 1H NMR (CDCl₃, 400 MHz) δ 10.40- 10.60 (m, 1H), 9.74 (s, 1H), 8.71 (d, 1H, J = 1.0 Hz), 8.14 (d, 1H, J = 7.8 Hz), 8.00-8.05 (m, 1H), 7.60-7.70 (m, 1H), 3.11 (q, 2H, J = 7.5 Hz), 2.30 (t, 3H, J = 18.9 Hz), 1.50-1.55 (m, 3H). |
| | N-(1-(6-(1,1-difluoroethyl)pyridin-2-yl)- | |
| | 3-ethyl-1H-pyrazolo[4,3-c]pyridin-6- | |
| | yl)acetamide-2,2,2-d3 | |
| 793 | 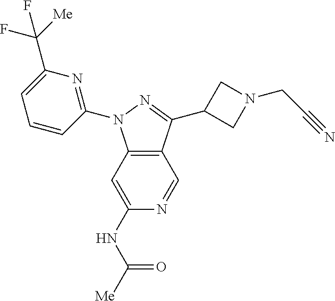 [SMILES: CC(=O)Nc1cc2c(cn1)c(C1CN(CC#N)C1)nn2-c1cccc(C(C)(F)F)n1] | LCMS m/z = 412 [M + H]⁺ 1H NMR (400 MHz, MeOD-d₄) δ 9.23-9.47 (m, 1H), 9.05 (d, J = 1.00 Hz, 1H), 8.17-8.28 (m, 1H), 8.07- 8.16 (m, 1H), 7.62 (dd, J = 0.75, 7.53 Hz, 1H), 4.28-4.38 (m, 1H), 4.15 (t, J = 8.16 Hz, 2H), 4.00-4.09 (m, 2H), 3.92 (s, 2H), 2.20-2.30 (m, 6H). |
| | N-(3-(1-(cyanomethyl)azetidin-3-y1)-1-(6- | |
| | (1,1-difluoroethyl)pyridin-2-y1)-1H- | |
| | pyrazolo[4,3-c]pyridin-6-yl)acetamide | |
| 794 | 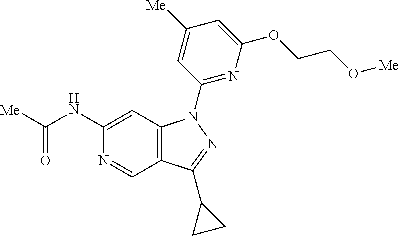 [SMILES: COCCOc1cc(C)cc(-n2nc(C3CC3)c3cnc(NC(C)=O)cc32)n1] | LCMS m/z = 382 [M + H]⁺ |
| | N-(3-cyclopropyl-1-(6-(2- | |
| | methoxyethoxy)-4-methylpyridin-2-yl)- | |
| | 1H-pyrazolo[4,3-c]pyridin-6-yl)acetamide | |
| 795 | 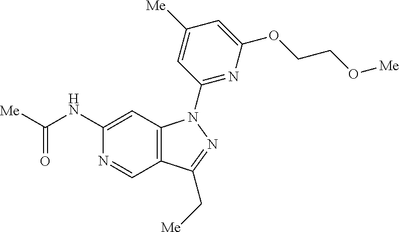 [SMILES: CCc1nn(-c2cc(C)cc(OCCOC)n2)c2cc(NC(C)=O)ncc12] | LCMS m/z = 370 [M + H]⁺ |
| | N-(3-ethyl-1-(6-(2-methoxyethoxy)-4- | |
| | methylpyridin-2-y1)-1H-pyrazolo[4,3- | |
| | c]pyridin-6-yl)acetamide | |
| 796 | 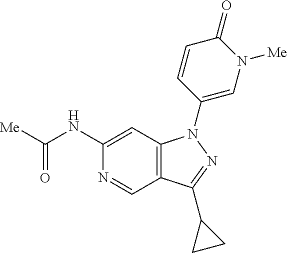 [SMILES: CC(=O)Nc1cc2c(cn1)c(C1CC1)nn2-c1ccc(=O)n(C)c1] | LCMS m/z = 324 [M + H]⁺ |
| | N-(3-cyclopropyl-1-(1-methyl-6-oxo-1,6- | |
| | dihydropyridin-3-yl)-1H-pyrazolo[4,3- | |
| | c]pyridin-6-yl)acetamide | |
| 797 | 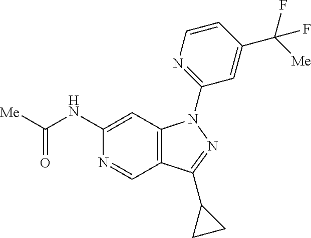 [SMILES: CC(=O)Nc1cc2c(cn1)c(C1CC1)nn2-c1cc(C(C)(F)F)ccn1] | LCMS m/z = 358 [M + H]⁺ |
| | N-(3-cyclopropyl-1-(4-(1,1- | |
| | difluoroethyl)pyridin-2-y1)-1H- | |
| | pyrazolo[4,3-c]pyridin-6-yl)acetamide | |
| 798 | 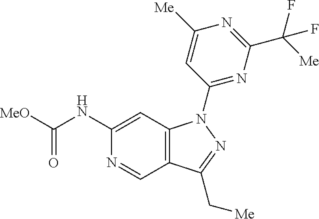 [SMILES: CCc1nn(-c2cc(C)nc(C(C)(F)F)n2)c2cc(NC(=O)OC)ncc12] | LCMS m/z = 377 [M + H]⁺ |
| | methyl (1-(2-(1,1-difluoroethyl)-6- | |
| | methylpyrimidin-4-yl)-3-ethyl-1H- | |
| | pyrazolo[4,3-c]pyridin-6-yl)carbamate | |
| 799 | 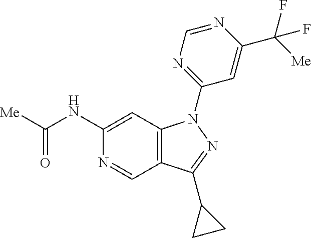 [SMILES: CC(=O)Nc1cc2c(cn1)c(C1CC1)nn2-c1cc(C(C)(F)F)ncn1] | LCMS m/z = 359 [M + H]⁺ |
| | N-(3-cyclopropyl-1-(6-(1,1- | |
| | difluoroethyl)pyrimidin-4-yl)-1H- | |
| | pyrazolo[4,3-c]pyridin-6-yl)acetamide | |
| 800 | 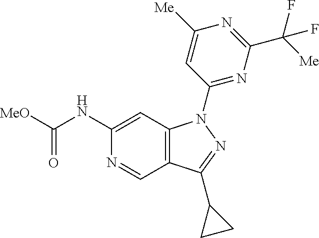 [SMILES: COC(=O)Nc1cc2c(cn1)c(C1CC1)nn2-c1cc(C)nc(C(C)(F)F)n1] | LCMS m/z = 389 [M + H]⁺ |
| | methyl (3-cyclopropyl-1-(2-(1,1- | |
| | difluoroethyl)-6-methylpyrimidin-4-yl)- | |
| | 1H-pyrazolo[4,3-c]pyridin-6-yl)carbamate | |
| 801 | 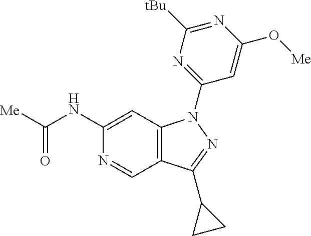 [SMILES: COc1cc(-n2nc(C3CC3)c3cnc(NC(C)=O)cc32)nc(C(C)(C)C)n1] | LCMS m/z = 381 [M + H]⁺ |
| | N-(1-(2-(tert-butyl)-6-methoxypyrimidin- | |
| | 4-yl)-3-cyclopropyl-1H-pyrazolo[4,3- | |
| | c]pyridin-6-yl)acetamide | |
| 802 | 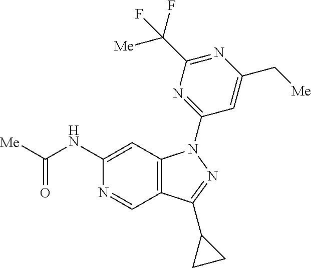 [SMILES: CCc1cc(-n2nc(C3CC3)c3cnc(NC(C)=O)cc32)nc(C(C)(F)F)n1] | LCMS m/z = 387.2 [M + H]⁺ 1H NMR (CDCl₃, 400 MHz): δ (ppm) 9.73 (s, 1H), 9.59-9.70 (m, 1H), 8.80 (s, 1H), 7.80 (s, 1H), 2.98 (q, J = 7.5 Hz, 2H), 2.24-2.38 (m, 7H), 1.39-1.45 (m, 3H), 1.25-1.31 (m, 4H) |
| | N-(3-cyclopropyl-1-(2-(1,1- | |
| | difluoroethyl)-6-ethylpyrimidin-4-yl)-1H- | |
| | pyrazolo[4,3-c]pyridin-6-yl)acetamide | |
| 803 | 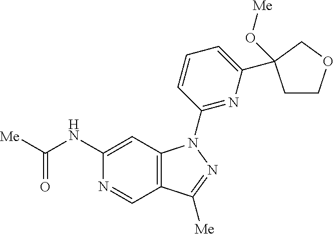 [SMILES: COC1(c2cccc(-n3nc(C)c4cnc(NC(C)=O)cc43)n2)CCOC1] | LCMS m/z = 368 [M + H]⁺ 1H NMR (400 MHz, MeOD-d₄) δ 9.21 (s, 1H), 8.83 (s, 1H), 7.97 (td, J = 7.69, 15.00 Hz, 2H), 7.42-7.50 (m, 1H), 4.33 (d, J = 9.79 Hz, 1H), 4.12-4.26 (m, 2H), 4.10 (d, J = 9.54 Hz, 1H), 3.26 (s, 3H), 2.87 (td, J = 8.82, 13.49 Hz, 1H), 2.67 (s, 3H), 2.57-2.65 (m, 1H), 2.24 (s, 3H). |
| | N-(1-(6-(3-methoxytetrahydrofuran-3- | |
| | yl)pyridin-2-yl)-3-methyl-1H- | |
| | pyrazolo[4,3-c]pyridin-6-yl)acetamide | |
| 804 | 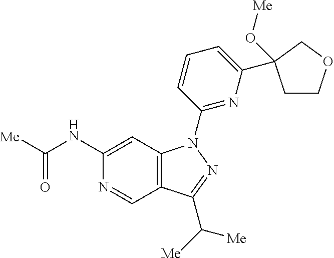 [SMILES: COC1(c2cccc(-n3nc(C(C)C)c4cnc(NC(C)=O)cc43)n2)CCOC1] | LCMS m/z = 396 [M + H]⁺ 1H NMR (400 MHz, MeOD-d₄) δ 9.01 (br d, J = 5.77 Hz, 2H), 7.96-8.09 (m, 2H), 7.50 (dd, J = 1.38, 6.90 Hz, 1H), 4.33 (br d, J = 9.54 Hz, 1H), 4.08-4.27 (m, 3H), 3.53 (td, J = 6.90, 13.80 Hz, 1H), 3.25 (s, 3H), 2.79 (td, J = 8.91, 13.30 Hz, 1H), 2.56-2.70 (m, 1H), 2.28 (s, 3H), 1.53 (d, J = 7.03 Hz, 6H). |
| | N-(3-isopropyl-1-(6-(3- | |
| | methoxytetrahydrofuran-3-y1)pyridin-2- | |
| | y1)-1H-pyrazolo[4,3-c]pyridin-6- | |
| | yl)acetamide | |
| 805 | 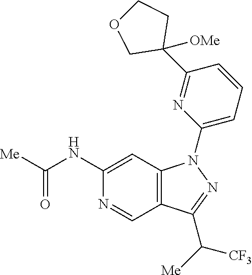 [SMILES: COC1(c2cccc(-n3nc(C(C)C(F)(F)F)c4cnc(NC(C)=O)cc43)n2)CCOC1] | LCMS m/z = 450 [M + H]⁺ 1H NMR (400 MHz, CDCl₃) δ 12.34 (br s, 1H), 9.83 (s, 1H), 8.96 (s, 1H), 7.98-8.05 (m, 2H), 7.61 (dd, J = 2.64, 5.90 Hz, 1H), 4.37 (d, J = 9.54 Hz, 1H), 4.24-4.34 (m, 2H), 4.18 (d, J = 9.54 Hz, 1H), 4.10-4.15 (m, 1H), 3.31 (s, 3H), 2.88 (td, J = 8.88, 13.36 Hz, 1H), 2.57-2.68 (m, 1H), 2.38 (s, 3H), 1.80 (d, J = 7.03 Hz, 3H) |
| | N-(1-(6-(3-methoxytetrahydrofuran-3- | |
| | yl)pyridin-2-yl)-3-(1,1,1-trifluoropropan- | |
| | 2-y1)-1H-pyrazolo[4,3-c]pyridin-6- | |
| | yl)acetamide | |
| 806 | 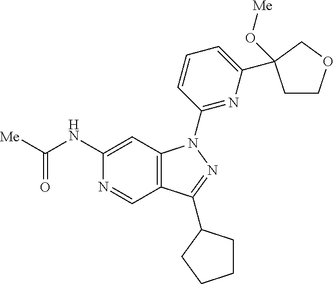 [SMILES: COC1(c2cccc(-n3nc(C4CCCC4)c4cnc(NC(C)=O)cc43)n2)CCOC1] | LCMS m/z = 422 [M + H]⁺ 1H NMR (400 MHz, MeOD-d₄) δ 9.02 (br s, 1H), 8.88 (br s, 1H), 7.97- 8.10 (m, 2H), 7.54 (d, J = 7.28 Hz, 1H), 4.34 (br d, J = 9.79 Hz, 1H), 4.10-4.23 (m, 3H), 3.66 (td, J = 8.06, 16.25 Hz, 1H), 3.25 (s, 3H), 2.74 (td, J = 8.78, 13.30 Hz, 1H), 2.57-2.68 (m, 1H), 2.22-2.34 (m, 5H), 2.02-2.15 (m, 2H), 1.90- 1.99 (m, 2H), 1.79-1.89 (m, 2H). |
| | N-(3-cyclopentyl-1-(6-(3- | |
| | methoxytetrahydrofuran-3-yl)pyridin-2- | |
| | yl)-1H-pyrazolo[4,3-c]pyridin-6- | |
| | yl)acetamide | |
| 807 | 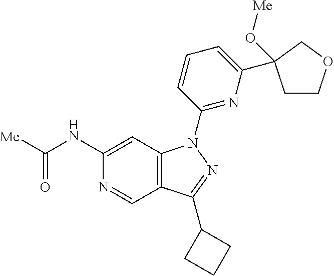 [SMILES: COC1(c2cccc(-n3nc(C4CCC4)c4cnc(NC(C)=O)cc43)n2)CCOC1] | LCMS m/z = 408 [M + H]⁺ 1H NMR (400 MHz, MeOD-d₄) δ 8.99 (s, 1H), 8.81 (s, 1H), 8.04-8.14 (m, 2H), 7.56 (dd, J = 3.39, 5.14 Hz, 1H), 4.34 (d, J = 9.79 Hz, 1H), 4.06- 4.25 (m, 4H), 3.25 (s, 3H), 2.68- 2.79 (m, 1H), 2.55-2.67 (m, 5H), 2.32 (s, 3H), 2.22-2.29 (m, 1H), 2.03-2.15 (m, 1H). |
| | N-(3-cyclobutyl-1-(6-(3- | |
| | methoxytetrahydrofuran-3-yl)pyridin-2- | |
| | yl)-1H-pyrazolo[4,3-c]pyridin-6- | |
| | yl)acetamide | |
| 808 | 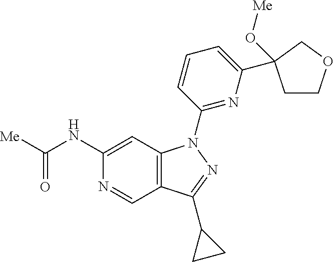 [SMILES: COC1(c2cccc(-n3nc(C4CC4)c4cnc(NC(C)=O)cc43)n2)CCOC1] | LCMS m/z = 394 [M + H]⁺ 1H NMR (400 MHz, MeOD-d₄) δ 9.22 (br d, J = 4.02 Hz, 1H), 8.86 (s, 1H), 7.92-8.00 (m, 1H), 7.84-7.91 (m, 1H), 7.43 (dd, J = 0.75, 7.53 Hz, 1H), 4.32 (dd, J = 1.25, 9.79 Hz, 1H), 4.19-4.25 (m, 1H), 4.15 (dt, J = 6.90, 8.34 Hz, 1H), 4.08 (d, J = 9.79 Hz, 1H), 3.25 (s, 3H), 2.82- 2.91 (m, 1H), 2.56-2.65 (m, 1H), 2.40 (tt, J = 5.36, 7.94 Hz, 1H), 2.23 (s, 3H), 1.15-1.21 (m, 4H). |
| | N-(3-cyclopropyl-1-(6-(3- | |
| | methoxytetrahydrofuran-3-yl)pyridin-2- | |
| | yl)-1H-pyrazolo[4,3-c]pyridin-6- | |
| | yl)acetamide | |
| 809 | 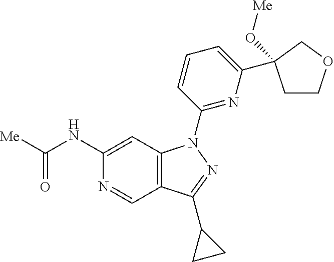 [SMILES: CO[C@@]1(c2cccc(-n3nc(C4CC4)c4cnc(NC(C)=O)cc43)n2)CCOC1] | LCMS m/z = 394 [M + H]⁺ 1H NMR (400 MHz, MeOD-d₄) δ 9.26 (s, 1H), 8.86 (d, J = 1.00 Hz, 1H), 7.93-7.99 (m, 1H), 7.86-7.90 (m, 1H), 7.43 (dd, J = 0.88, 7.40 Hz, 1H), 4.32 (dd, J = 1.25, 9.79 Hz, 1H), 4.19-4.26 (m, 1H), 4.15 (dt, J = 6.90, 8.34 Hz, 1H), 4.08 (d, J = 9.79 Hz, 1H), 3.26 (s, 3H), 2.88 (td, J = 8.82, 13.49 Hz, 1H), 2.61 (dddd, J = 1.25, 3.83, 6.87, 13.40 Hz, 1H), 2.36-2.44 (m, 1H), 2.23 (s, 3H), 1.15-1.22 (m, 4H). |
| | or | |
| | 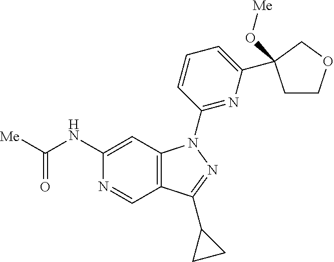 [SMILES: CO[C@]1(c2cccc(-n3nc(C4CC4)c4cnc(NC(C)=O)cc43)n2)CCOC1] | |
| | (R)-N-(3-cyclopropyl-1-(6-(3- | |
| | methoxytetrahydrofuran-3-yl)pyridin-2- | |
| | yl)-1H-pyrazolo[4,3-c]pyridin-6- | |
| | yl)acetamide or (S)-N-(3-cyclopropyl-1- | |
| | (6-(3-methoxytetrahydrofuran-3- | |
| | yl)pyridin-2-yl)-1H-pyrazolo[4,3- | |
| | c]pyridin-6-yl)acetamide | |
| 810 | 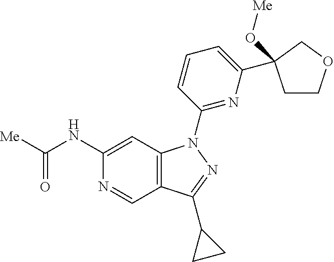 [SMILES: CO[C@]1(c2cccc(-n3nc(C4CC4)c4cnc(NC(C)=O)cc43)n2)CCOC1] | LCMS m/z = 394 [M + H]⁺ 1H NMR (400 MHz, MeOD-d₄) δ 9.26 (s, 1H), 8.86 (d, J = 1.00 Hz, 1H), 7.93-7.99 (m, 1H), 7.86-7.91 (m, 1H), 7.43 (dd, J = 1.00, 7.53 Hz, 1H), 4.32 (dd, J = 1.13, 9.66 Hz, 1H), 4.19-4.25 (m, 1H), 4.11-4.18 (m, 1H), 4.08 (d, J = 9.54 Hz, 1H), 3.26 (s, 3H), 2.88 (td, J = 8.88, 13.36 Hz, 1H), 2.61 (dddd, J = 1.25, 3.89, 6.87, 13.33 Hz, 1H), 2.36- 2.44 (m, 1H), 2.23 (s, 3H), 1.15- 1.22 (m, 4H). |
| | or | |
| | 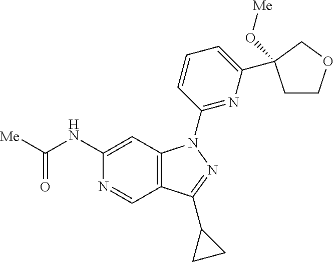 [SMILES: CO[C@@]1(c2cccc(-n3nc(C4CC4)c4cnc(NC(C)=O)cc43)n2)CCOC1] | |
| | (S)-N-(3-cyclopropyl-1-(6-(3- | |
| | methoxytetrahydrofuran-3-yl)pyridin-2- | |
| | yl)-1H-pyrazolo[4,3-c]pyridin-6- | |
| | yl)acetamide or (R)-N-(3-cyclopropyl-1- | |
| | (6-(3-methoxytetrahydrofuran-3- | |
| | yl)pyridin-2-yl)-1H-pyrazolo[4,3- | |
| | c]pyridin-6-yl)acetamide | |
| 811 | 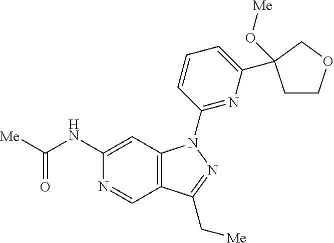 [SMILES: CCc1nn(-c2cccc(C3(OC)CCOC3)n2)c2cc(NC(C)=O)ncc12] | LCMS m/z = 382 [M + H]⁺ |
| | N-(3-ethyl-1-(6-(3- | |
| | methoxytetrahydrofuran-3-y1)pyridin-2- | |
| | yl)-1H-pyrazolo[4,3-c]pyridin-6- | |
| | yl)acetamide | |
| 812 | 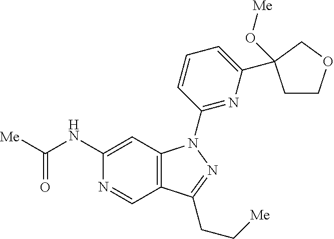 [SMILES: CCCc1nn(-c2cccc(C3(OC)CCOC3)n2)c2cc(NC(C)=O)ncc12] | LCMS m/z = 396 [M + H]⁺ 1H NMR (500 MHz, DMSO-d₆) δ 10.64-10.74 (m, 1H), 9.24 (s, 1H), 8.97 (d, J = 0.61 Hz, 1H), 8.04 (t, J = 7.93 Hz, 1H), 7.88 (dd, J = 0.61, 8.24 Hz, 1H), 7.42 (dd, J = 0.69, 7.55 Hz, 1H), 4.27 (dd, J = 1.07, 9.77 Hz, 1H), 4.07 (dt, J = 3.66, 8.32 Hz, 1H), 3.97-4.04 (m, 1H), 3.91 (d, J = 9.77 Hz, 1H), 3.14 (s, 3H), 3.02 (t, J = 7.55 Hz, 2H), 2.74 (td, J = 8.77, 13.28 Hz, 1H), 2.53- 2.58 (m, 1H), 2.15 (s, 3H), 1.80- 1.90 (m, 2H), 1.00 (t, J = 7.32 Hz, 3H). |
| | N-(1-(6-(3-methoxytetrahydrofuran-3- | |
| | yl)pyridin-2-yl)-3-propyl-1H- | |
| | pyrazolo[4,3-c]pyridin-6-yl)acetamide | |
| 813 | 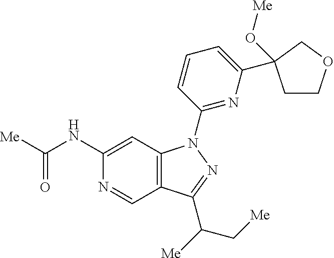 [SMILES: CCC(C)c1nn(-c2cccc(C3(OC)CCOC3)n2)c2cc(NC(C)=O)ncc12] | LCMS m/z = 410 [M + H]⁺ 1H NMR (500 MHz, DMSO-d₆) δ 10.68 (s, 1H), 9.25 (s, 1H), 9.01 (d, J = 0.92 Hz, 1H), 8.04 (t, J = 7.93 Hz, 1H), 7.89 (dd, J = 0.61, 8.24 Hz, 1H), 7.42 (dd, J = 0.69, 7.55 Hz, 1H), 4.25-4.35 (m, 1H), 4.04-4.11 (m, 1H), 3.96-4.03 (m, 1H), 3.88- 3.95 (m, 1H), 3.21-3.34 (m, 1H), 3.05-3.20 (m, 3H), 2.74 (td, J = 8.79, 13.24 Hz, 1H), 2.53-2.59 (m, 1H), 1.87-1.99 (m, 1H), 1.80 (td, J = 6.85, 13.92 Hz, 1H), 1.44 (d, J = 7.02 Hz, 3H), 0.90 (t, J = 7.40 Hz, 3H) |
| | N-(3-(sec-butyl)-1-(6-(3- | |
| | methoxytetrahydrofuran-3-yl)pyridin-2- | |
| | yl)-1H-pyrazolo[4,3-c]pyridin-6- | |
| | yl)acetamide | |
| 814 | 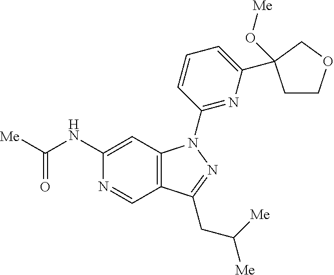 [SMILES: COC1(c2cccc(-n3nc(CC(C)C)c4cnc(NC(C)=O)cc43)n2)CCOC1] | LCMS m/z = 410 [M + H]⁺ 1H NMR (500 MHz, DMSO-d₆) δ 10.69 (s, 1H), 9.25 (d, J = 0.61 Hz, 1H), 8.96 (d, J = 1.07 Hz, 1H), 7.97- 8.16 (m, 1H), 7.89 (dd, J = 0.76, 8.24 Hz, 1H), 7.42 (dd, J = 0.76, 7.63 Hz, 1H), 4.27 (dd, J = 1.22, 9.77 Hz, 1H), 4.04-4.13 (m, 1H), 3.96-4.03 (m, 1H), 3.87-3.95 (m, 1H), 3.14 (s, 3H), 2.92 (d, J = 7.17 Hz, 2H), 2.75 (td, J = 8.83, 13.31 Hz, 1H), 2.53-2.59 (m, 1H), 2.08- 2.25 (m, 4H), 0.99 (d, J = 6.71 Hz, 6H). |
| | N-(3-isobutyl-1-(6-(3- | |
| | methoxytetrahydrofuran-3-y1)pyridin-2- | |
| | yl)-1H-pyrazolo[4,3-c]pyridin-6- | |
| | yl)acetamide | |
| 815 | 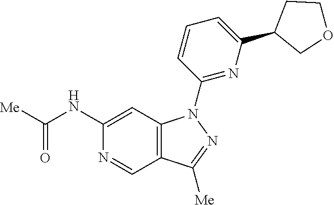 [SMILES: CC(=O)Nc1cc2c(cn1)c(C)nn2-c1cccc([C@H]2CCOC2)n1] | LCMS m/z = 338 [M + H]⁺ 1H NMR (400 MHz, MeOD-d₄) δ 9.34 (s, 1H), 8.80 (d, J = 1.00 Hz, 1H), 7.79-7.90 (m, 2H), 7.20 (dd, J = 1.38, 6.90 Hz, 1H), 4.30 (t, J = 8.03 Hz, 1H), 4.22 (dt, J = 5.02, 8.16 Hz, 1H), 3.96-4.04 (m, 2H), 3.72 (quin, J = 7.97 Hz, 1H), 2.66 (s, 3H), 2.42-2.60 (m, 2H), 2.23 (s, 3H). |
| | or | |
| | 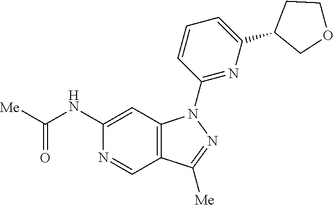 [SMILES: CC(=O)Nc1cc2c(cn1)c(C)nn2-c1cccc([C@@H]2CCOC2)n1] | |
| | (R)-N-(3-methyl-1-(6-(tetrahydrofuran-3- | |
| | yl)pyridin-2-yl)-1H-pyrazolo[4,3- | |
| | c]pyridin-6-yl)acetamide or (S)-N-(3- | |
| | methyl-1-(6-(tetrahydrofuran-3- | |
| | yl)pyridin-2-yl)-1H-pyrazolo[4,3- | |
| | c]pyridin-6-yl)acetamide | |
| 816 | 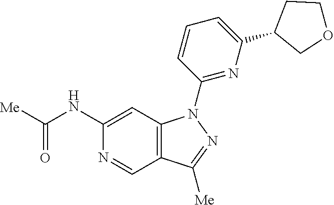 [SMILES: CC(=O)Nc1cc2c(cn1)c(C)nn2-c1cccc([C@@H]2CCOC2)n1] | LCMS m/z = 338 [M + H]⁺ 1H NMR (400 MHz, MeOD-d₄) δ 9.32 (s, 1H), 8.77 (d, J = 1.00 Hz, 1H), 7.77-7.88 (m, 2H), 7.18 (dd, J = 1.00, 7.03 Hz, 1H), 4.30 (t, J = 8.03 Hz, 1H), 4.22 (dt, J = 5.02, 8.16 Hz, 1H), 3.95-4.04 (m, 2H), 3.71 (quin, J = 8.03 Hz, 1H), 2.64 (s, 3H), 2.41-2.59 (m, 2H), 2.23 (s, 3H). |
| | or | |
| | 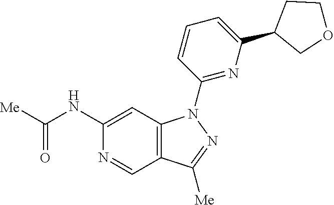 [SMILES: CC(=O)Nc1cc2c(cn1)c(C)nn2-c1cccc([C@H]2CCOC2)n1] | |
| | (S)-N-(3-methyl-1-(6-(tetrahydrofuran-3- | |
| | yl)pyridin-2-yl)-1H-pyrazolo[4,3- | |
| | c]pyridin-6-yl)acetamide or (R)-N-(3- | |
| | methyl-1-(6-(tetrahydrofuran-3- | |
| | yl)pyridin-2-yl)-1H-pyrazolo[4,3- | |
| | c]pyridin-6-yl)acetamide | |
| 817 | 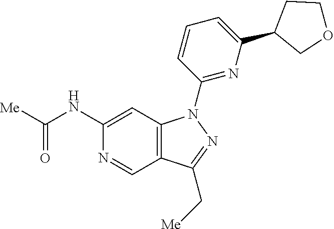 [SMILES: CCc1nn(-c2cccc([C@H]3CCOC3)n2)c2cc(NC(C)=O)ncc12] | LCMS m/z = 352 [M + H]⁺ 1H NMR (400 MHz, MeOD-d₄) δ 9.35 (s, 1H), 8.83 (d, J = 1.00 Hz, 1H), 7.80-7.90 (m, 2H), 7.20 (dd, J = 2.26, 6.02 Hz, 1H), 4.30 (t, J = 8.03 Hz, 1H), 4.22 (dt, J = 5.02, 8.16 Hz, 1H), 3.96-4.04 (m, 2H), 3.72 (quin, J = 8.03 Hz, 1H), 3.09 (q, J = 7.53 Hz, 2H), 2.41-2.60 (m, 2H), 2.23 (s, 3H), 1.47 (t, J = 7.53 Hz, 3H). |
| | or | |
| | 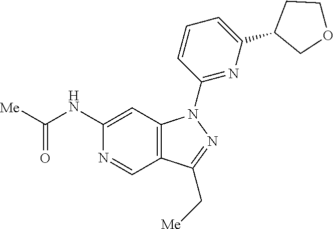 [SMILES: CCc1nn(-c2cccc([C@@H]3CCOC3)n2)c2cc(NC(C)=O)ncc12] | |
| | (R)-N-(3-ethyl-1-(6-(tetrahydrofuran-3- | |
| | yl)pyridin-2-yl)-1H-pyrazolo[4,3- | |
| | c]pyridin-6-yl)acetamide or (S)-N-(3- | |
| | ethyl-1-(6-(tetrahydrofuran-3-y1)pyridin- | |
| | 2-yl)-1H-pyrazolo[4,3-c]pyridin-6- | |
| | yl)acetamide | |
| 818 | 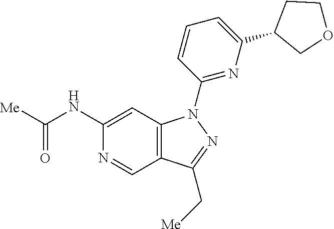 [SMILES: CCc1nn(-c2cccc([C@@H]3CCOC3)n2)c2cc(NC(C)=O)ncc12] | LCMS m/z = 352 [M + H]⁺ 1H NMR (400 MHz, CDCl₃) δ 9.56 (s, 1H), 9.40 (br s, 1H), 8.70 (s, 1H), 7.83-7.88 (m, 1H), 7.75-7.82 (m, 1H), 7.12 (d, J = 7.28 Hz, 1H), 4.33 (t, J = 8.03 Hz, 1H), 4.25 (dt, J = 5.77, 7.91 Hz, 1H), 3.99-4.08 (m, 2H), 3.71 (quin, J = 7.91 Hz, 1H), 3.08 (q, J = 7.61 Hz, 2H), 2.49- 2.58 (m, 2H), 2.30 (s, 3H), 1.49 (t, J = 7.65 Hz, 3H). |
| | or | |
| | 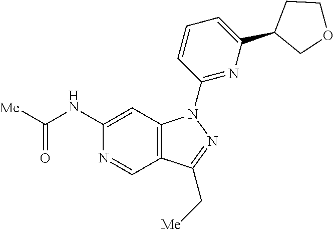 [SMILES: CCc1nn(-c2cccc([C@H]3CCOC3)n2)c2cc(NC(C)=O)ncc12] | |
| | (S)-N-(3-ethyl-1-(6-(tetrahydrofuran-3- | |
| | yl)pyridin-2-yl)-1H-pyrazolo[4,3- | |
| | c]pyridin-6-yl)acetamide or (R)-N-(3- | |
| | ethyl-1-(6-(tetrahydrofuran-3-y1)pyridin- | |
| | 2-y1)-1H-pyrazolo[4,3-c]pyridin-6- | |
| | yl)acetamide | |
| 819 | 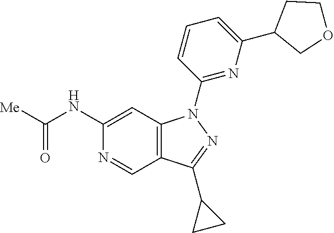 [SMILES: CC(=O)Nc1cc2c(cn1)c(C1CC1)nn2-c1cccc(C2CCOC2)n1] | LCMS m/z = 364 [M + H]⁺ 1H NMR (400 MHz, MeOD-d₄) δ 9.25 (s, 1H), 8.89 (d, J = 0.75 Hz, 1H), 7.82-7.88 (m, 1H), 7.77-7.81 (m, 1H), 7.20 (d, J = 7.53 Hz, 1H), 4.29 (t, J = 8.03 Hz, 1H), 4.21 (dt, J = 5.40, 7.97 Hz, 1H), 3.96-4.04 (m, 2H), 3.72 (t, J = 8.03 Hz, 1H), 2.45-2.55 (m, 2H), 2.36-2.43 (m, 1H), 2.24 (s, 3H), 1.16-1.22 (m, 4H). |
| | N-(3-cyclopropyl-1-(6-(tetrahydrofuran-3- | |
| | yl)pyridin-2-yl)-1H-pyrazolo[4,3- | |
| | c]pyridin-6-yl)acetamide | |
| 820 | 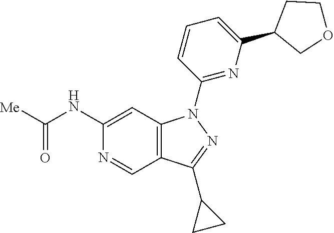 [SMILES: CC(=O)Nc1cc2c(cn1)c(C1CC1)nn2-c1cccc([C@H]2CCOC2)n1] | LCMS m/z = 364 [M + H]⁺ 1H NMR (400 MHz, MeOD-d₄) δ 9.34 (s, 1H), 8.85 (d, J = 1.00 Hz, 1H), 7.74-7.87 (m, 2H), 7.15-7.21 (m, 1H), 4.29 (t, J = 7.91 Hz, 1H), 4.22 (dt, J = 5.02, 8.16 Hz, 1H), 3.96- 4.03 (m, 2H), 3.66-3.76 (m, 1H), 2.35-2.59 (m, 3H), 1.14-1.21 (m, 4H). |
| | or | |
| | 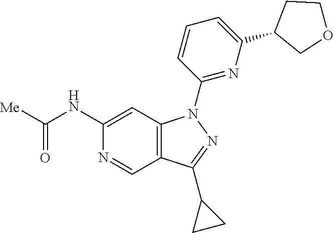 [SMILES: CC(=O)Nc1cc2c(cn1)c(C1CC1)nn2-c1cccc([C@@H]2CCOC2)n1] | |
| | (R)-N-(3-cyclopropyl-1-(6- | |
| | (tetrahydrofuran-3-y1)pyridin-2-yl)-1H- | |
| | pyrazolo[4,3-c]pyridin-6-yl)acetamide or | |
| | (S)-N-(3-cyclopropyl-1-(6- | |
| | (tetrahydrofuran-3-y1)pyridin-2-y1)-1H- | |
| | pyrazolo[4,3-c]pyridin-6-yl)acetamide | |
| 821 | 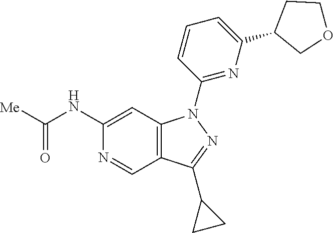 [SMILES: CC(=O)Nc1cc2c(cn1)c(C1CC1)nn2-c1cccc([C@@H]2CCOC2)n1] | LCMS m/z = 364 [M + H]⁺ 1H NMR (400 MHz, MeOD-d₄) δ 9.34 (s, 1H), 8.85 (d, J = 1.00 Hz, 1H), 7.75-7.87 (m, 2H), 7.18 (d, J = 6.78 Hz, 1H), 4.29 (t, J = 7.91 Hz, 1H), 4.22 (dt, J = 5.02, 8.16 Hz, 1H), 3.96-4.03 (m, 2H), 3.71 (quin, J = 8.03 Hz, 1H), 2.43-2.60 (m, 2H), 2.35-2.43 (m, 1H), 2.23 (s, 3H), 1.11-1.25 (m, 4H). |
| | or | |
| | 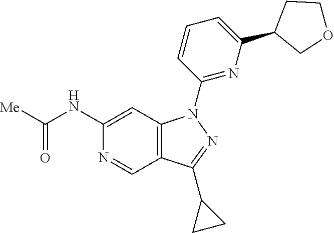 [SMILES: CC(=O)Nc1cc2c(cn1)c(C1CC1)nn2-c1cccc([C@H]2CCOC2)n1] | |
| | (S)-N-(3-cyclopropyl-1-(6- | |
| | (tetrahydrofuran-3-yl)pyridin-2-yl)-1H- | |
| | pyrazolo[4,3-c]pyridin-6-yl)acetamide or | |
| | (R)-N-(3-cyclopropyl-1-(6- | |
| | (tetrahydrofuran-3-yl)pyridin-2-y1)-1H- | |
| | pyrazolo[4,3-c]pyridin-6-yl)acetamide | |
| 822 | 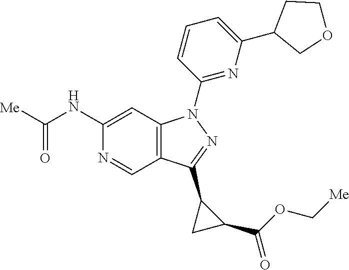 [SMILES: CCOC(=O)[C@H]1C[C@H]1c1nn(-c2cccc(C3CCOC3)n2)c2cc(NC(C)=O)ncc12] | LCMS m/z = 436 [M + H]⁺ 1H NMR (400 MHz, MeOD-d₄) δ 9.35 (s, 1H), 8.88 (d, J = 1.00 Hz, 1H), 7.82-7.89 (m, 1H), 7.77-7.82 (m, 1H), 7.20 (d, J = 7.03 Hz, 1H), 4.29 (t, J = 7.91 Hz, 1H), 4.18-4.26 (m, 3H), 3.95-4.03 (m, 2H), 3.71 (quin, J = 8.03 Hz, 1H), 2.93 (ddd, J = 4.02, 6.27, 9.03 Hz, 1H), 2.37-2.57 (m, 3H), 2.23 (s, 3H), 1.68-1.79 (m, 2H), 1.31 (t, J = 7.15 Hz, 3H). |
| | ethyl (1S,2R)-2-(6-acetamido-1-(6- | |
| | (tetrahydrofuran-3-yl)pyridin-2-yl)-1H- | |
| | pyrazolo[4,3-c]pyridin-3-yl)cyclopropane- | |
| | 1-carboxylate | |
| 823 | 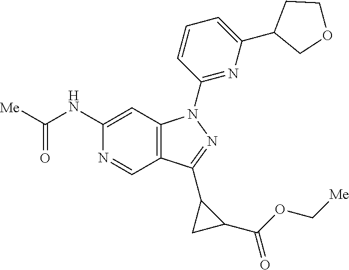 [SMILES: CCOC(=O)C1CC1c1nn(-c2cccc(C3CCOC3)n2)c2cc(NC(C)=O)ncc12] | LCMS m/z = 436 [M + H]⁺ 1H NMR (400 MHz, MeOD-d₄) δ 9.35 (s, 1H), 8.84 (d, J = 0.75 Hz, 1H), 7.80-7.91 (m, 2H), 7.21 (dd, J = 1.13, 7.15 Hz, 1H), 4.30 (t, J = 7.91 Hz, 1H), 4.22 (dt, J = 5.02, 8.16 Hz, 1H), 3.96-4.03 (m, 2H), 3.77-3.86 (m, 2H), 3.72 (quin, J = 8.03 Hz, 1H), 2.85 (dt, J = 7.28, 8.78 Hz, 1H), 2.42-2.60 (m, 2H), 2.31-2.39 (m, 1H), 2.22 (s, 3H), 1.99 (ddd, J = 4.77, 6.02, 7.03 Hz, 1H), 1.60 (dt, J = 4.64, 8.22 Hz, 1H), 0.88 (t, J = 7.15 Hz, 3H). |
| | ethyl 2-(6-acetamido-1-(6- | |
| | (tetrahydrofuran-3-yl)pyridin-2-yl)-1H- | |
| | pyrazolo[4,3-c]pyridin-3-yl)cyclopropane- | |
| | 1-carboxylate | |
| 824 | 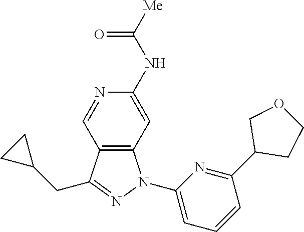 [SMILES: CC(=O)Nc1cc2c(cn1)c(CC1CC1)nn2-c1cccc(C2CCOC2)n1] | LCMS m/z = 378 [M + H]⁺ 1H NMR (400 MHz, CDCl₃) δ 12.31 (br s, 1H), 9.90 (d, J = 0.75 Hz, 1H), 8.68 (s, 1H), 7.82-7.92 (m, 2H), 7.23 (d, J = 8.03 Hz, 1H), 5.91 (tdd, J = 6.71, 10.26, 16.97 Hz, 1H), 5.05-5.16 (m, 2H), 4.29-4.37 (m, 1H), 4.25 (dt, J = 5.27, 8.16 Hz, 1H), 4.00-4.08 (m, 2H), 3.73 (quin, J = 7.84 Hz, 1H), 3.16 (t, J = 7.40 Hz, 2H), 2.62-2.70 (m, 2H), 2.49-2.56 (m, 2H), 2.39 (s, 3H). |
| | N-(3-(cyclopropylmethyl)-1-(6- | |
| | (tetrahydrofuran-3-y1)pyridin-2-y1)-1H- | |
| | pyrazolo[4,3-c]pyridin-6-yl)acetamide | |
| 825 | 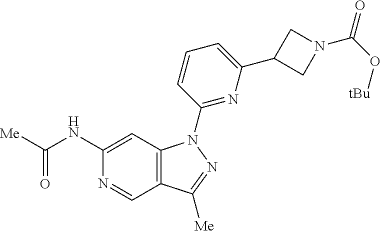 [SMILES: CC(=O)Nc1cc2c(cn1)c(C)nn2-c1cccc(C2CN(C(=O)OC(C)(C)C)C2)n1] | LCMS m/z = 423 [M + H]⁺ |
| | tert-butyl 3-(6-(6-acetamido-3-methyl-1H- | |
| | pyrazolo[4,3-c]pyridin-1-yl)pyridin-2- | |
| | yl)azetidine-1-carboxylate | |
| 826 | 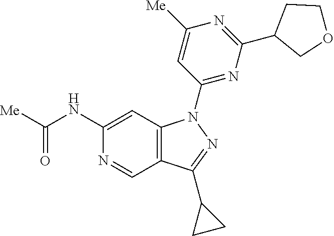 [SMILES: CC(=O)Nc1cc2c(cn1)c(C1CC1)nn2-c1cc(C)nc(C2CCOC2)n1] | LCMS m/z = 379 [M + H]⁺ 1H NMR (400 MHz, CDCl₃) δ 9.47 (s, 1H), 8.74 (d, 1H, J = 1.0 Hz), 8.26 (br s, 1H), 7.58 (s, 1H), 4.30-4.40 (m, 1H), 4.1-4.3 (m, 2H), 4.00- 4.10 (m, 1H), 3.82 (quin, 1H, J = 7.8 Hz), 2.69 (qd, 1H, J = 7.5, 12.4 Hz), 2.58 (s, 3H), 2.50-2.52 (m, 1H), 2.20-2.30 (m, 4H), 1.20- 1.22 (m, 4H). |
| | N-(3-cyclopropyl-1-(6-methyl-2- | |
| | (tetrahydrofuran-3-yl)pyrimidin-4-yl)-1H- | |
| | pyrazolo[4,3-c]pyridin-6-yl)acetamide | |
| 827 | 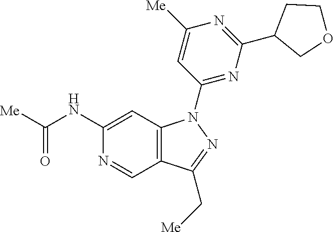 [SMILES: CCc1nn(-c2cc(C)nc(C3CCOC3)n2)c2cc(NC(C)=O)ncc12] | LCMS m/z = 367 [M + H]⁺ 1H NMR (400 MHz, CDCl₃) δ 9.4-9.6 (m, 1H), 8.72 (d, 1H, J = 1.0 Hz), 8.20-8.40 (m, 1H), 7.65 (s, 1H), 4.30-4.40 (m, 1H), 4.10-4.30 (m, 2H), 4.00-4.10 (m, 1H), 3.80-3.90 (m, 1H), 3.07 (q, 2H, J = 7.6 Hz), 2.60-2.80 (m, 1H), 2.60-2.62 (m, 3H), 2.50-2.60 (m, 1H), 2.28 (s, 3H), 1.49 (t, 3H, J = 7.5 Hz). |
| | N-(3-ethyl-1-(6-methyl-2- | |
| | (tetrahydrofuran-3-y1)pyrimidin-4-y1)-1H- | |
| | pyrazolo[4,3-c]pyridin-6-yl)acetamide | |
| 828 | 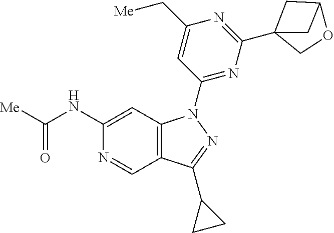 [SMILES: CCc1cc(-n2nc(C3CC3)c3cnc(NC(C)=O)cc32)nc(C23COC(C2)C3)n1] | LCMS m/z = 405 [M + H]⁺ 1H NMR (400 MHz, CDCl₃) δ 9.54 (s, 1H), 8.73 (d, 1H, J = 1.0 Hz), 8.3- 8.5 (m, 1H), 7.59 (s, 1H), 4.76 (s, 1H), 4.24 (s, 2H), 2.84 (q, 2H, J = 7.7 Hz), 2.55 (d, 2H, J = 5.0 Hz), 2.20-2.30 (m, 4H), 2.10-2.20 (m, 2H), 1.36 (t, 3H, J = 7.5 Hz), 1.20- 1.30 (m, 2H), 1.20 (td, 2H, J = 2.6, 8.1 Hz). |
| | N-(1-(2-(2-oxabicyclo[2.1.1]hexan-4-yl)- | |
| | 6-ethylpyrimidin-4-yl)-3-cyclopropyl-1H- | |
| | pyrazolo[4,3-c]pyridin-6-yl)acetamide | |
| 829 | 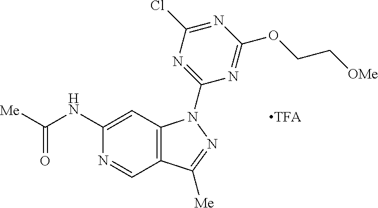 [SMILES: COCCOc1nc(Cl)nc(-n2nc(C)c3cnc(NC(C)=O)cc32)n1] | LCMS m/z = 378 [M + H]⁺ |
| | N-(1-(4-chloro-6-(2-methoxyethoxy)- | |
| | 1,3,5-triazin-2-y1)-3-methyl-1H- | |
| | pyrazolo[4,3-c]pyridin-6-yl)acetamide | |
| | trifluoroacetate | |
| 830 | 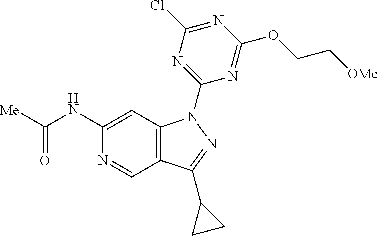 [SMILES: COCCOc1nc(Cl)nc(-n2nc(C3CC3)c3cnc(NC(C)=O)cc32)n1] | LCMS m/z = 404 [M + H]⁺ |
| | N-(1-(4-chloro-6-(2-methoxyethoxy)- | |
| | 1,3,5-triazin-2-y1)-3-cyclopropyl-1H- | |
| | pyrazolo[4,3-c]pyridin-6-yl)acetamide | |
| 831 | 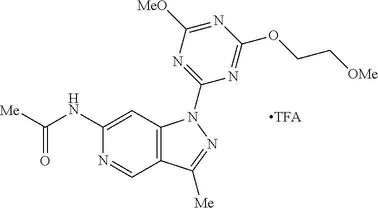 [SMILES: COCCOc1nc(OC)nc(-n2nc(C)c3cnc(NC(C)=O)cc32)n1] | LCMS m/z = 374 [M + H]⁺ |
| | N-(1-(4-methoxy-6-(2-methoxyethoxy)- | |
| | 1,3,5-triazin-2-yl)-3-methyl-1H- | |
| | pyrazolo[4,3-c]pyridin-6-yl)acetamide | |
| | trifluoroacetate | |
| 832 | 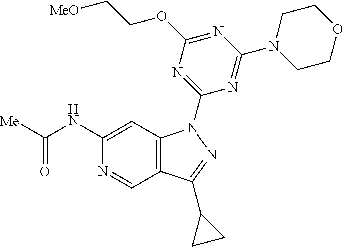 [SMILES: COCCOc1nc(N2CCOCC2)nc(-n2nc(C3CC3)c3cnc(NC(C)=O)cc32)n1] | LCMS m/z = 455 [M + H]⁺ |
| | N-(3-cyclopropyl-1-(4-(2- | |
| | methoxyethoxy)-6-morpholino-1,3,5- | |
| | triazin-2-y1)-1H-pyrazolo[4,3-c]pyridin-6- | |
| | yl)acetamide | |
| 833 | 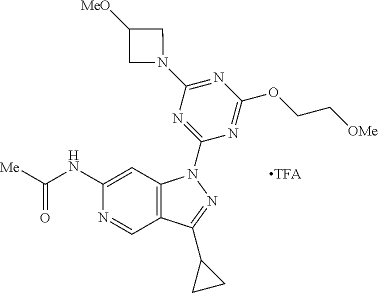 [SMILES: COCCOc1nc(N2CC(OC)C2)nc(-n2nc(C3CC3)c3cnc(NC(C)=O)cc32)n1] | LCMS m/z = 455 [M + H]⁺ |
| | N-(3-cyclopropyl-1-(4-(3- | |
| | methoxyazetidin-1-yl)-6-(2- | |
| | methoxyethoxy)-1,3,5-triazin-2-yl)-1H- | |
| | pyrazolo[4,3-c]pyridin-6-yl)acetamide | |
| | trifluoroacetate | |
| 834 | 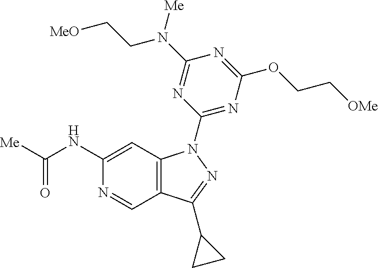 [SMILES: COCCOc1nc(N(C)CCOC)nc(-n2nc(C3CC3)c3cnc(NC(C)=O)cc32)n1] | LCMS m/z = 457 [M + H]⁺ |
| | N-(3-cyclopropyl-1-(4-(2- | |
| | methoxyethoxy)-6-((2- | |
| | methoxyethyl)(methyl)amino)-1,3,5- | |
| | triazin-2-yl)-1H-pyrazolo[4,3-c]pyridin-6- | |
| | yl)acetamide | |
| 835 | 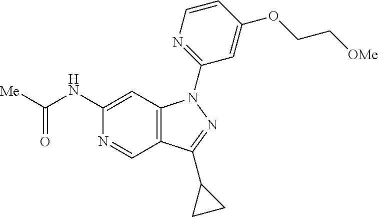 [SMILES: COCCOc1ccnc(-n2nc(C3CC3)c3cnc(NC(C)=O)cc32)c1] | LCMS m/z = 368 [M + H]⁺ |
| | N-(3-cyclopropyl-1-(4-(2- | |
| | methoxyethoxy)pyridin-2-y1)-1H- | |
| | pyrazolo[4,3-c]pyridin-6-yl)acetamide | |
| 836 | 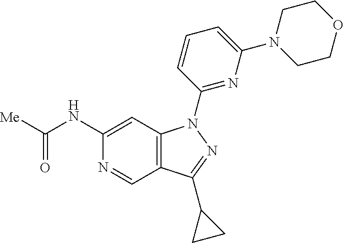 [SMILES: CC(=O)Nc1cc2c(cn1)c(C1CC1)nn2-c1cccc(N2CCOCC2)n1] | LCMS m/z = 379 [M + H]⁺ |
| | N-(3-cyclopropyl-1-(6- | |
| | morpholinopyridin-2-yl)-1H- | |
| | pyrazolo[4,3-c]pyridin-6-yl)acetamide | |
| 837 | 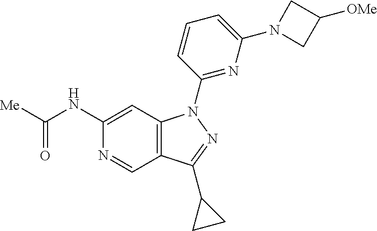 [SMILES: COC1CN(c2cccc(-n3nc(C4CC4)c4cnc(NC(C)=O)cc43)n2)C1] | LCMS m/z = 379 [M + H]⁺ |
| | N-(3-cyclopropyl-1-(6-(3- | |
| | methoxyazetidin-1-yl)pyridin-2-y1)-1H- | |
| | pyrazolo[4,3-c]pyridin-6-yl)acetamide | |
| 838 | 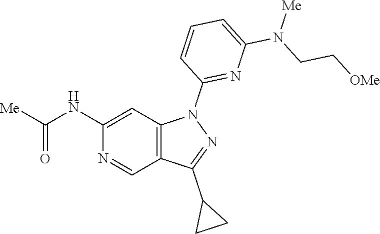 [SMILES: COCCN(C)c1cccc(-n2nc(C3CC3)c3cnc(NC(C)=O)cc32)n1] | LCMS m/z = 381 [M + H]⁺ |
| | N-(3-cyclopropyl-1-(6-((2- | |
| | methoxyethyl)(methyl)amino)pyridin-2- | |
| | yl)-1H-pyrazolo[4,3-c]pyridin-6- | |
| | yl)acetamide | |
| 839 | 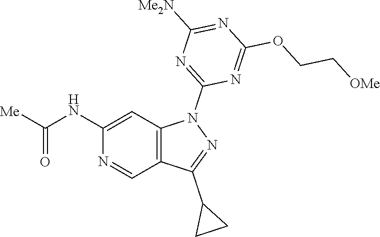 [SMILES: COCCOc1nc(N(C)C)nc(-n2nc(C3CC3)c3cnc(NC(C)=O)cc32)n1] | LCMS m/z = 413 [M + H]⁺ |
| | N-(3-cyclopropyl-1-(4-(dimethylamino)- | |
| | 6-(2-methoxyethoxy)-1,3,5-triazin-2-yl)- | |
| | 1H-pyrazolo[4,3-c]pyridin-6-yl)acetamide | |
| 840 | 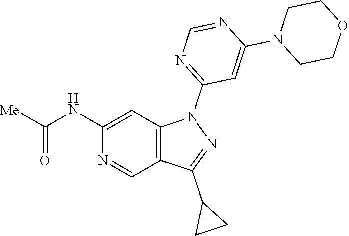 [SMILES: CC(=O)Nc1cc2c(cn1)c(C1CC1)nn2-c1cc(N2CCOCC2)ncn1] | LCMS m/z = 380 [M + H]⁺ |
| | N-(3-cyclopropyl-1-(6- | |
| | morpholinopyrimidin-4-yl)-1H- | |
| | pyrazolo[4,3-c]pyridin-6-yl)acetamide | |
| 841 | 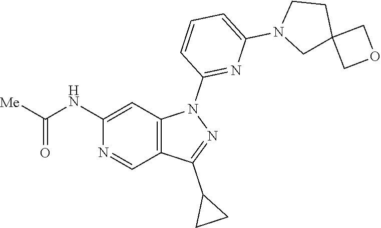 [SMILES: CC(=O)Nc1cc2c(cn1)c(C1CC1)nn2-c1cccc(N2CCC3(COC3)C2)n1] | LCMS m/z = 405 [M + H]⁺ |
| | N-(1-(6-(2-oxa-6-azaspiro[3.4]octan-6- | |
| | yl)pyridin-2-yl)-3-cyclopropyl-1H- | |
| | pyrazolo[4,3-c]pyridin-6-yl)acetamide | |
| 842 | 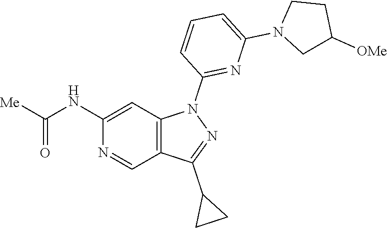 [SMILES: COC1CCN(c2cccc(-n3nc(C4CC4)c4cnc(NC(C)=O)cc43)n2)C1] | LCMS m/z = 393 [M + H]⁺ |
| | N-(3-cyclopropyl-1-(6-(3- | |
| | methoxypyrrolidin-1-yl)pyridin-2-yl)-1H- | |
| | pyrazolo[4,3-c]pyridin-6-yl)acetamide | |
| 843 | 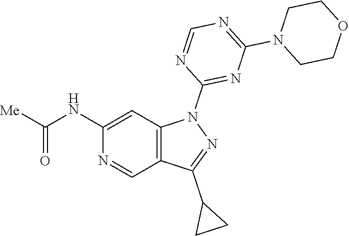 [SMILES: CC(=O)Nc1cc2c(cn1)c(C1CC1)nn2-c1ncnc(N2CCOCC2)n1] | LCMS m/z = 381 [M + H]⁺ |
| | N-(3-cyclopropyl-1-(4-morpholino-1,3,5- | |
| | triazin-2-y1)-1H-pyrazolo[4,3-c]pyridin-6- | |
| | yl)acetamide | |
| 844 | 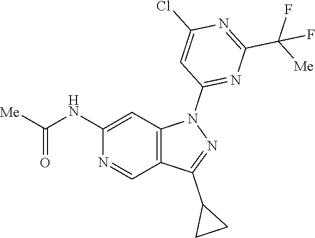 [SMILES: CC(=O)Nc1cc2c(cn1)c(C1CC1)nn2-c1cc(Cl)nc(C(C)(F)F)n1] | LCMS m/z = 393.1 [M + H]⁺ 1H NMR (600 MHz, DMSO-d₆) ppm 1.12-1.31 (m, 4 H), 2.16 (s, 3 H), 2.19-2.29 (m, 3 H), 2.51-2.54 (m, 1 H), 7.98 (s, 1 H), 9.03 (s, 1 H), 9.35 (s, 1 H), 10.83 (s, 1 H). |
| | N-(1-(6-chloro-2-(1,1- | |
| | difluoroethyl)pyrimidin-4-y1)-3- | |
| | cyclopropyl-1H-pyrazolo[4,3-c]pyridin-6- | |
| | yl)acetamide | |
| 845 | 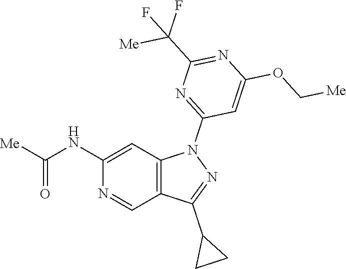 [SMILES: CCOc1cc(-n2nc(C3CC3)c3cnc(NC(C)=O)cc32)nc(C(C)(F)F)n1] | LCMS m/z = 403.3 [M + H]⁺ 1H NMR (600 MHz, DMSO-d₆) (ppm) 10.81-10.70 (m, 1H), 9.36- 9.27 (m, 1H), 9.02-8.93 (m, 1H), 7.21-7.11 (m, 1H), 4.48 (q, J = 7.0 Hz, 2H), 2.49-2.45 (m, 1H), 2.21 (s, 3H), 2.15 (s, 3H), 1.38 (s, 3H), 1.21-1.11 (m, 4H) |
| | N-(3-cyclopropyl-1-(2-(1,1- | |
| | difluoroethyl)-6-ethoxypyrimidin-4-y1)- | |
| | 1H-pyrazolo[4,3-c]pyridin-6-yl)acetamide | |
| 846 | 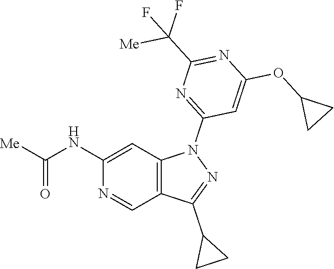 [SMILES: CC(=O)Nc1cc2c(cn1)c(C1CC1)nn2-c1cc(OC2CC2)nc(C(C)(F)F)n1] | LCMS m/z = 415.3 [M + H]⁺ 1H NMR (600 MHz, DMSO-d₆) (ppm)10.79-10.71 (m, 1H), 9.39- 9.29 (m, 1H), 9.03-8.94 (m, 1H), 7.36-7.26 (m, 1H), 4.42 (s, 1H), 2.48-2.43 (m, 1H), 2.21 (t, J = 19.3 Hz, 3H), 2.15 (s, 3H), 1.21-1.12 (m, 4H), 0.92-0.75 (m, 4H) |
| | N-(1-(6-cyclopropoxy-2-(1,1- | |
| | difluoroethyl)pyrimidin-4-y1)-3- | |
| | cyclopropyl-1H-pyrazolo[4,3-c]pyridin-6- | |
| | yl)acetamide | |
| 847 | 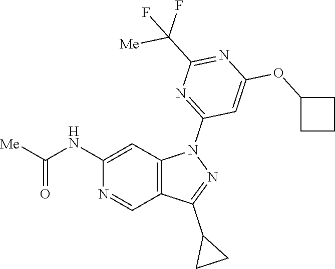 [SMILES: CC(=O)Nc1cc2c(cn1)c(C1CC1)nn2-c1cc(OC2CCC2)nc(C(C)(F)F)n1] | LCMS m/z = 429.3 [M + H]⁺ 1H NMR (600 MHz, DMSO-d₆) (ppm) 10.78-10.69 (m, 1H), 9.38- 9.29 (m, 1H), 9.04-8.94 (m, 1H), 7.16-7.09 (m, 1H), 5.27 (t, J = 7.4 Hz, 1H), 2.48-2.40 (m, 3H), 2.25- 2.16 (m, 4H), 2.15 (s, 5H), 1.87- 1.76 (m, 1H), 1.75-1.63 (m, 1H), 1.21-1.12 (m, 4H) |
| | N-(1-(6-cyclobutoxy-2-(1,1- | |
| | difluoroethyl)pyrimidin-4-y1)-3- | |
| | cyclopropyl-1H-pyrazolo[4,3-c]pyridin-6- | |
| | yl)acetamide | |
| 848 | 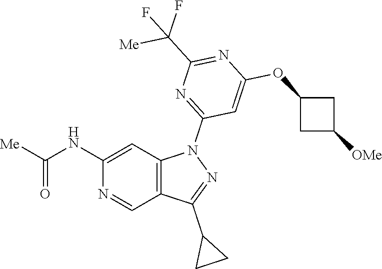 [SMILES: CO[C@H]1C[C@@H](Oc2cc(-n3nc(C4CC4)c4cnc(NC(C)=O)cc43)nc(C(C)(F)F)n2)C1] | LCMS m/z = 459.2 [M + H]⁺ 1H NMR (600 MHz, DMSO-d₆) (ppm) 10.74 (s, 1H), 9.33 (s, 1H), 8.99 (s, 1H), 7.15 (s, 1H), 4.96 (t, J = 7.1 Hz, 1H), 3.70 (t, J = 6.9 Hz, 1H), 3.17 (s, 2H), 2.93-2.82 (m, 2H), 2.48-2.43 (m, 1H), 2.20 (t, J 3 19.3 Hz, 3H), 2.15 (s, 3H), 2.06- 1.94 (m, 2H), 1.21-1.13 (m, 4H) |
| | N-(3-cyclopropyl-1-(2-(1,1- | |
| | difluoroethyl)-6-((1s,3s)-3- | |
| | methoxycyclobutoxy)pyrimidin-4-y1)-1H- | |
| | pyrazolo[4,3-c]pyridin-6-yl)acetamide | |
| 849 | 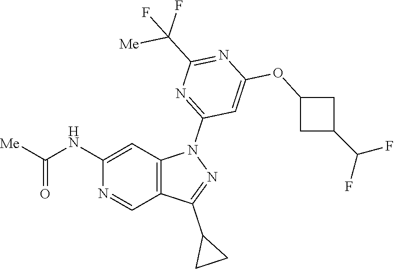 [SMILES: CC(=O)Nc1cc2c(cn1)c(C1CC1)nn2-c1cc(OC2CC(C(F)F)C2)nc(C(C)(F)F)n1] | LCMS m/z = 479.2 [M + H]⁺ 1H NMR (600 MHz, DMSO-d₆) (ppm) 10.74 (s, 1H), 9.32 (s, 1H), 8.99 (s, 1H), 7.15 (s, 1H), 6.11 (d, J = 3.3 Hz, 1H), 5.42-5.17 (m, 1H), 2.62-2.54 (m, 3H), 2.49-2.45 (m, 1H), 2.20 (s, 4H), 2.14 (s, 4H), 1.31-1.09 (m, 4H) |
| | N-(3-cyclopropyl-1-(2-(1,1- | |
| | difluoroethyl)-6-(3- | |
| | (difluoromethyl)cyclobutoxy)pyrimidin-4- | |
| | yl)-1H-pyrazolo[4,3-c]pyridin-6- | |
| | yl)acetamide | |
| 850 | 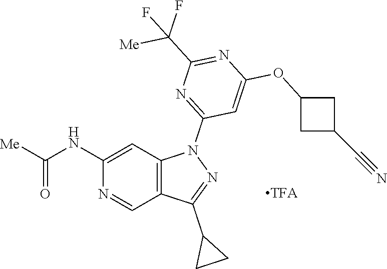 [SMILES: CC(=O)Nc1cc2c(cn1)c(C1CC1)nn2-c1cc(OC2CC(C#N)C2)nc(C(C)(F)F)n1] | LCMS m/z = 454.2 [M + H]⁺ 1H NMR (600 MHz, DMSO-d₆) (ppm) 10.71 (s, 1H), 9.33-9.24 (m, 1H), 8.95 (s, 1H), 7.15-7.10 (m, 1H), 5.23 (t, J = 7.3 Hz, 1H), 3.19- 3.11 (m, 1H), 2.93-2.82 (m, 2H), 2.80-2.55 (m, 1H), 2.45-2.40 (m, 2H), 2.16 (t, J = 19.3 Hz, 3H), 2.11 (s, 3H), 1.14 (br s, 5H) |
| | N-(1-(6-(3-cyanocyclobutoxy)-2-(1,1- | |
| | difluoroethyl)pyrimidin-4-y1)-3- | |
| | cyclopropyl-1H-pyrazolo[4,3-c]pyridin-6- | |
| | yl)acetamide trifluoroacetate | |
| 851 | 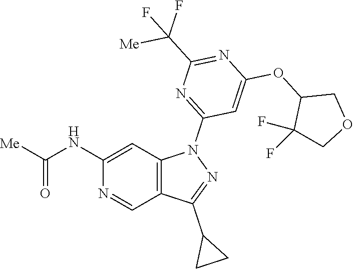 [SMILES: CC(=O)Nc1cc2c(cn1)c(C1CC1)nn2-c1cc(OC2COCC2(F)F)nc(C(C)(F)F)n1] | LCMS m/z = 481.2 [M + H]⁺ 1H NMR (600 MHz, DMSO-d₆) (ppm) 10.74 (s, 1H), 9.32 (s, 1H), 8.99 (s, 1H), 7.29 (s, 1H), 5.83- 5.66 (m, 1H), 4.40 (dd, J = 6.0, 10.7 Hz, 1H), 4.15-3.93 (m, 4H), 2.47-2.43 (m, 1H), 2.19 (t, J = 19.1 Hz, 3H), 2.13 (s, 4H), 1.21-1.05 (m, 4H) |
| | N-(3-cyclopropyl-1-(2-(1,1- | |
| | difluoroethyl)-6-((4,4- | |
| | difluorotetrahydrofuran-3- | |
| | yl)oxy)pyrimidin-4-yl)-1H-pyrazolo[4,3- | |
| | c]pyridin-6-yl)acetamide | |
| 852 | 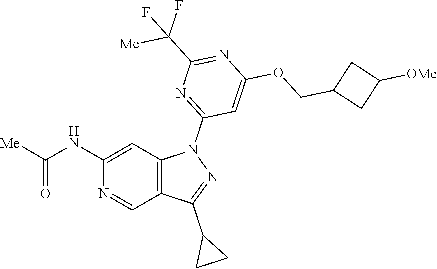 [SMILES: COC1CC(COc2cc(-n3nc(C4CC4)c4cnc(NC(C)=O)cc43)nc(C(C)(F)F)n2)C1] | LCMS m/z = 473.2 [M + H]⁺ 1H NMR (600 MHz, DMSO-d₆) (ppm) 10.74 (s, 1H), 9.33 (s, 1H), 8.99 (d, J = 1.1 Hz, 1H), 7.16 (s, 1H), 4.49-4.34 (m, 2H), 4.08-3.69 (m, 1H), 3.15-3.07 (m, 2H), 2.48- 2.43 (m, 1H), 2.37-2.26 (m, 2H), 2.26-2.17 (m, 3H), 2.15 (s, 4H), 2.09-2.01 (m, 1H), 1.78-1.65 (m, 2H), 1.22-1.12 (m, 4H) |
| | N-(3-cyclopropyl-1-(2-(1,1- | |
| | difluoroethyl)-6-((3- | |
| | methoxycyclobutyl)methoxy)pyrimidin-4- | |
| | yl)-1H-pyrazolo[4,3-c]pyridin-6- | |
| | yl)acetamide | |
| 853 | 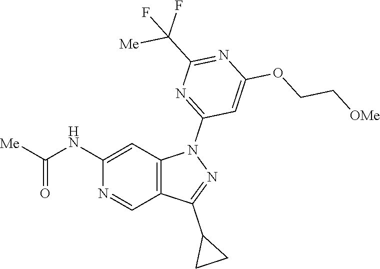 [SMILES: COCCOc1cc(-n2nc(C3CC3)c3cnc(NC(C)=O)cc32)nc(C(C)(F)F)n1] | LCMS m/z = 433.2 [M + H]⁺ 1H NMR (600 MHz, DMSO-d₆) (ppm) 10.74 (s, 1H), 9.37-9.29 (m, 1H), 9.02-8.93 (m, 1H), 7.22-7.17 (m, 1H), 4.58-4.51 (m, 2H), 3.74- 3.65 (m, 2H), 2.48-2.43 (m, 1H), 2.21 (t, J = 19.3 Hz, 3H), 2.14 (s, 3H), 1.21-1.12 (m, 4H) |
| | N-(3-cyclopropyl-1-(2-(1,1- | |
| | difluoroethyl)-6-(2- | |
| | methoxyethoxy)pyrimidin-4-yl)-1H- | |
| | pyrazolo[4,3-c]pyridin-6-yl)acetamide | |
| 854 | 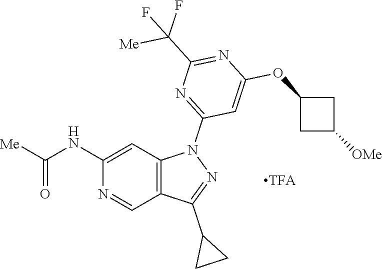 [SMILES: CO[C@H]1C[C@H](Oc2cc(-n3nc(C4CC4)c4cnc(NC(C)=O)cc43)nc(C(C)(F)F)n2)C1] | LCMS m/z = 459.2 [M + H]⁺ 1H NMR (500 MHz, DMSO-d₆) (ppm) 10.78 (s, 1H), 9.34 (s, 1H), 9.01 (s, 1H), 7.15 (s, 1H), 5.40 (s, 1H), 4.14-4.01 (m, 1H), 2.49-2.34 (m, 4H), 2.29-2.16 (m, 4H), 2.15 (s, 3H), 1.31-1.08 (m, 7H) |
| | N-(3-cyclopropyl-1-(2-(1,1- | |
| | difluoroethyl)-6-((1r,3r)-3- | |
| | methoxycyclobutoxy)pyrimidin-4-yl)-1H- | |
| | pyrazolo[4,3-c]pyridin-6-yl)acetamide | |
| 855 | 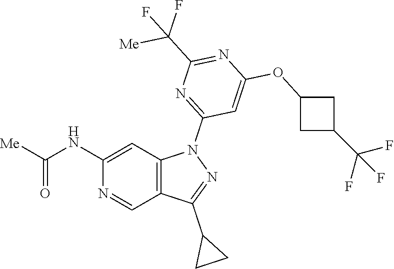 [SMILES: CC(=O)Nc1cc2c(cn1)c(C1CC1)nn2-c1cc(OC2CC(C(F)(F)F)C2)nc(C(C)(F)F)n1] | LCMS m/z = 497.2 [M + H]⁺ 1H NMR (500 MHz, DMSO-d₆) (ppm) 10.79 (s, 1H), 9.34 (s, 1H), 9.01 (s, 1H), 7.22-7.15 (m, 1H), 5.47-5.24 (m, 1H), 2.81-2.56 (m, 2H), 2.30-2.17 (m, 4H), 2.17-2.12 (m, 3H), 1.23-1.15 (m, 4H) |
| | N-(3-cyclopropyl-1-(2-(1,1- | |
| | difluoroethyl)-6-(3- | |
| | (trifluoromethyl)cyclobutoxy)pyrimidin- | |
| | 4-y1)-1H-pyrazolo[4,3-c]pyridin-6- | |
| | yl)acetamide | |
| 856 | 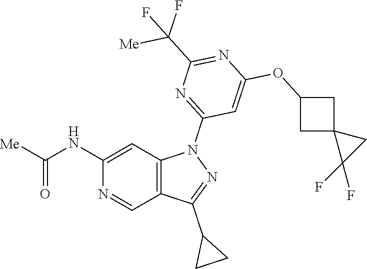 [SMILES: CC(=O)Nc1cc2c(cn1)c(C1CC1)nn2-c1cc(OC2CC3(C2)CC3(F)F)nc(C(C)(F)F)n1] | LCMS m/z = 491.2 [M + H]⁺ 1H NMR (500 MHz, DMSO-d₆) (ppm) 10.86-10.71 (m, 1H), 9.39- 9.30 (m, 1H), 9.05-8.96 (m, 1H), 7.22-7.17 (m, 1H), 5.60-5.32 (m, 1H), 2.77-2.59 (m, 1H), 2.48-2.41 (m, 1H), 2.21 (t, J = 19.2 Hz, 4H), 2.15 (s, 3H), 1.62-1.46 (m, 2H), 1.19 (s, 5H) |
| | N-(3-cyclopropyl-1-(2-(1,1- | |
| | difluoroethyl)-6-((1,1- | |
| | difluorospiro[2.3]hexan-5- | |
| | yl)oxy)pyrimidin-4-yl)-1H-pyrazolo[4,3- | |
| | c]pyridin-6-yl)acetamide | |
| 857 | 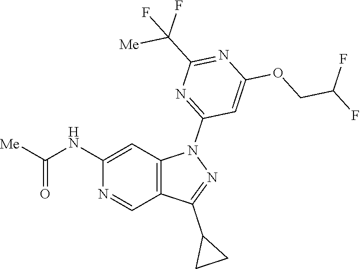 [SMILES: CC(=O)Nc1cc2c(cn1)c(C1CC1)nn2-c1cc(OCC(F)F)nc(C(C)(F)F)n1] | LCMS m/z = 439.1 [M + H]⁺ 1H NMR (500 MHz, DMSO-d₆) (ppm) 10.80 (s, 1H), 9.35 (s, 1H), 9.01 (s, 1H), 7.34-7.24 (m, 1H), 6.59-6.27 (m, 1H), 4.78 (dt, J = 3.1, 15.3 Hz, 2H), 2.23 (t, J = 19.2 Hz, 3H), 2.16 (s, 3H), 1.28-1.05 (m, 5H) |
| | N-(3-cyclopropyl-1-(6-(2,2- | |
| | difluoroethoxy)-2-(1,1- | |
| | difluoroethyl)pyrimidin-4-y1)-1H- | |
| | pyrazolo[4,3-c]pyridin-6-yl)acetamide | |
| 858 | 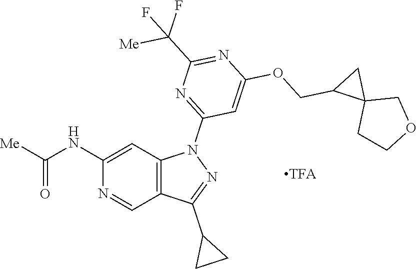 [SMILES: CC(=O)Nc1cc2c(cn1)c(C1CC1)nn2-c1cc(OCC2CC23CCOC3)nc(C(C)(F)F)n1] | LCMS m/z = 485.2 [M + H]⁺ 1H NMR (500 MHz, DMSO-d₆) (ppm) 10.78 (s, 1H), 9.35 (s, 1H), 9.01 (s, 1H), 7.18 (s, 1H), 4.70- 4.53 (m, 1H), 4.30-4.06 (m, 1H), 2.22 (s, 4H), 2.16 (s, 4H), 2.06- 1.98 (m, 1H), 1.95-1.88 (m, 1H), 1.87-1.79 (m, 1H), 1.60-1.39 (m, 1H) |
| | N-(1-(6-((5-oxaspiro[2.4]heptan-1- | |
| | yl)methoxy)-2-(1,1- | |
| | difluoroethyl)pyrimidin-4-yl)-3- | |
| | cyclopropyl-1H-pyrazolo[4,3-c]pyridin-6- | |
| | yl)acetamide trifluoroacetate | |
| 859 | 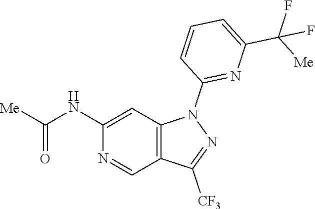 [SMILES: CC(=O)Nc1cc2c(cn1)c(C(F)(F)F)nn2-c1cccc(C(C)(F)F)n1] | LCMS m/z = 386 [M + H]⁺ 1H NMR (400 MHz, CDCl₃) δ 9.55 (s, 1H), 8.91 (s, 1H), 8.28 (br s, 1H), 8.16 (d, J = 8.28 Hz, 1H), 8.04 (t, J = 7.91 Hz, 1H), 7.68 (d, J = 7.28 Hz, 1H), 2.28-2.38 (m, 6H) |
| | N-(1-(6-(1,1-difluoroethyl)pyridin-2-y1)- | |
| | 3-(trifluoromethyl)-1H-pyrazolo[4,3- | |
| | c]pyridin-6-yl)acetamide | |
| 860 | 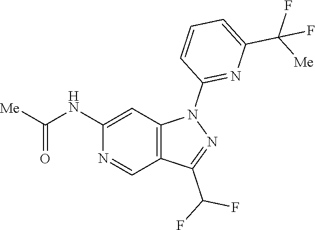 [SMILES: CC(=O)Nc1cc2c(cn1)c(C(F)F)nn2-c1cccc(C(C)(F)F)n1] | LCMS m/z = 368 [M + H]⁺ 1H NMR (400 MHz, CDCl₃) δ 12.03 (br s, 1H), 9.94 (s, 1H), 9.00 (s, 1H), 8.04-8.20 (m, 2H), 7.75 (dd, J = 1.00, 7.53 Hz, 1H), 6.92-7.22 (m, 1H), 2.39 (s, 3H), 2.30 (t, J = 18.95 Hz, 3H) |
| | N-(1-(6-(1,1-difluoroethyl)pyridin-2-yl)- | |
| | 3-(difluoromethyl)-1H-pyrazolo[4,3- | |
| | c]pyridin-6-yl)acetamide | |
| 861 | 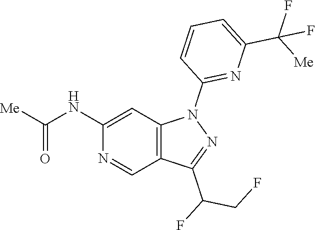 [SMILES: CC(=O)Nc1cc2c(cn1)c(C(F)CF)nn2-c1cccc(C(C)(F)F)n1] | LCMS m/z = 382 [M + H]⁺ 1H NMR (400 MHz, CDCl₃) δ 12.41 (br s, 1H), 9.93 (s, 1H), 8.99 (s, 1H), 8.02-8.17 (m, 2H), 7.73 (dd, J = 2.01, 6.53 Hz, 1H), 6.04-6.30 (m, 1H), 5.06-5.32 (m, 1H), 4.78- 5.05 (m, 1H), 2.40 (s, 3H), 2.29 (t, J = 18.82 Hz, 3H). |
| | N-(3-(1,2-difluoroethyl)-1-(6-(1,1- | |
| | difluoroethyl)pyridin-2-y1)-1H- | |
| | pyrazolo[4,3-c]pyridin-6-yl)acetamide | |
| 862 | 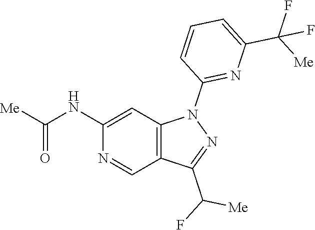 [SMILES: CC(=O)Nc1cc2c(cn1)c(C(C)F)nn2-c1cccc(C(C)(F)F)n1] | LCMS m/z = 364 [M + H]⁺ 1H NMR (400 MHz, MeOD-d₄) d 9.25 (br s, 1H), 9.01 (br s, 1H), 8.07-8.18 (m, 2H), 7.64 (dd, J = 2.38, 6.15 Hz, 1H), 6.05-6.29 (m, 1H), 2.17-2.34 (m, 6H), 1.86- 2.00 (m, 3H) |
| | N-(1-(6-(1,1-difluoroethyl)pyridin-2-y1)- | |
| | 3-(1-fluoroethyl)-1H-pyrazolo[4,3- | |
| | c]pyridin-6-yl)acetamide | |
| 863 | 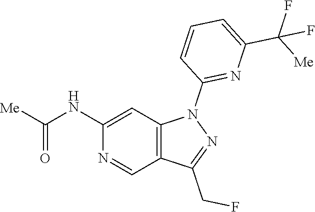 [SMILES: CC(=O)Nc1cc2c(cn1)c(CF)nn2-c1cccc(C(C)(F)F)n1] | LCMS m/z = 350 [M + H]⁺ 1H NMR (400 MHz, MeOD-d₄) δ 9.31-9.56 (m, 1H), 8.96 (br s, 1H), 8.11-8.18 (m, 2H), 7.62-7.66 (m, 1H), 5.69-5.95 (m, 2H), 2.21-2.31 (m, 6H). |
| | N-(1-(6-(1,1-difluoroethyl)pyridin-2-yl)- | |
| | 3-(fluoromethyl)-1H-pyrazolo[4,3- | |
| | c]pyridin-6-yl)acetamide | |
| 864 | 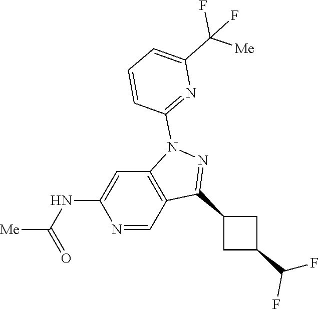 [SMILES: CC(=O)Nc1cc2c(cn1)c([C@H]1C[C@@H](C(F)F)C1)nn2-c1cccc(C(C)(F)F)n1] | LCMS m/z = 422 [M + H]⁺ 1H NMR (400 MHz, CDCl₃) d 10.72 (br d, J = 6.53 Hz, 1H), 9.78 (s, 1H), 8.70 (s, 1H), 8.17 (d, J = 8.03 Hz, 1H), 8.05 (t, J = 7.91 Hz, 1H), 7.63- 7.72 (m, 1H), 5.84-6.24 (m, 1H), 3.97-4.11 (m, 1H), 2.89-3.07 (m, 1H), 2.65-2.79 (m, 4H), 2.37 (s, 3H), 2.30 (t, J = 18.95 Hz, 3H) |
| | N-(1-(6-(1,1-difluoroethyl)pyridin-2-yl)- | |
| | 3-((1s,3s)-3-(difluoromethyl)cyclobutyl)- | |
| | 1H-pyrazolo[4,3-c]pyridin-6-yl)acetamide | |
| 865 | 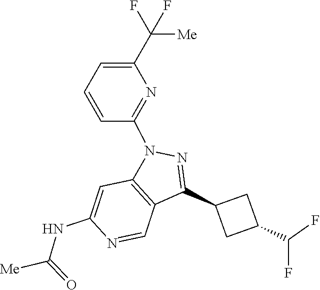 [SMILES: CC(=O)Nc1cc2c(cn1)c([C@H]1C[C@H](C(F)F)C1)nn2-c1cccc(C(C)(F)F)n1] | LCMS m/z = 422 [M + H]⁺ 1H NMR (400 MHz, CDCl₃) δ 10.43 (br s, 1H), 9.73 (s, 1H), 8.76 (s, 1H), 8.15 (d, J = 8.28 Hz, 1H), 7.98- 8.09 (m, 1H), 7.65 (d, J = 7.53 Hz, 1H), 5.68-6.07 (m, 1H), 3.94 (quin, J = 8.97 Hz, 1H), 2.89-3.08 (m, 1H), 2.59-2.74 (m, 4H), 2.36 (s, 3H), 2.30 (t, J = 18.95 Hz, 3H) |
| | N-(1-(6-(1,1-difluoroethyl)pyridin-2-yl)- | |
| | 3-((1r,3r)-3-(difluoromethyl)cyclobutyl)- | |
| | 1H-pyrazolo[4,3-c]pyridin-6-yl)acetamide | |
| 866 | 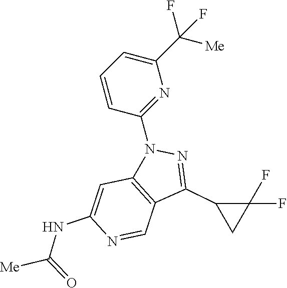 [SMILES: CC(=O)Nc1cc2c(cn1)c(C1CC1(F)F)nn2-c1cccc(C(C)(F)F)n1] | LCMS m/z = 394 [M + H]⁺ 1H NMR (400 MHz, CDCl₃) δ 12.29 (br s, 1H), 9.93 (s, 1H), 8.85 (s, 1H), 8.10-8.15 (m, 1H), 8.04-8.09 (m, 1H), 7.71 (d, J = 7.53 Hz, 1H), 3.11 (dt, J = 7.91, 10.98 Hz, 1H), 2.40 (s, 3H), 2.22-2.37 (m, 4H), 2.12-2.22 (m, 1H). |
| | N-(3-(2,2-difluorocyclopropyl)-1-(6-(1,1- | |
| | difluoroethyl)pyridin-2-y1)-1H- | |
| | pyrazolo[4,3-c]pyridin-6-yl)acetamide | |
| 867 | 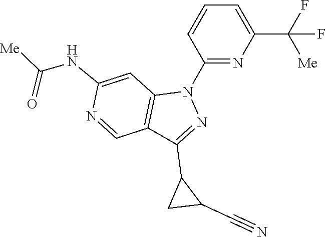 [SMILES: CC(=O)Nc1cc2c(cn1)c(C1CC1C#N)nn2-c1cccc(C(C)(F)F)n1] | LCMS m/z = 383 [M + H]⁺ 1H NMR (500 MHz, DMSO-d₆) δ 10.79 (s, 1H), 9.35 (d, J = 0.76 Hz, 1H), 9.12 (d, J = 1.07 Hz, 1H), 8.12- 8.25 (m, 1H), 8.06 (d, J = 8.39 Hz, 1H), 7.63 (dd, J = 0.69, 7.55 Hz, 1H), 2.52 (br s, 1H), 2.49 (br s, 1H), 2.20-2.33 (m, 3H), 2.16 (s, 3H), 1.85 (t, J = 7.63 Hz, 2H). |
| | N-(3-(2-cyanocyclopropyl)-1-(6-(1,1- | |
| | difluoroethyl)pyridin-2-yl)-1H- | |
| | pyrazolo[4,3-c]pyridin-6-yl)acetamide | |
| 868 | 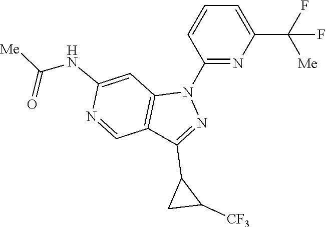 [SMILES: CC(=O)Nc1cc2c(cn1)c(C1CC1C(F)(F)F)nn2-c1cccc(C(C)(F)F)n1] | LCMS m/z = 426 [M + H]⁺ 1H NMR (400 MHz, CDCl₃) δ 12.30 (br s, 1H), 9.89 (s, 1H), 8.84 (s, 1H), 8.00-8.10 (m, 2H), 7.68 (dd, J = 2.51, 6.02 Hz, 1H), 2.63-2.73 (m, 1H), 2.42-2.43 (m, 1H), 2.39 (s, 3H), 2.29 (br t, J = 18.95 Hz, 3H), 1.68 (br t, J = 7.40 Hz, 2H) |
| | N-(1-(6-(1,1-difluoroethyl)pyridin-2-yl)- | |
| | 3-(2-(trifluoromethyl)cyclopropyl)-1H- | |
| | pyrazolo[4,3-c]pyridin-6-yl)acetamide | |
| 869 | 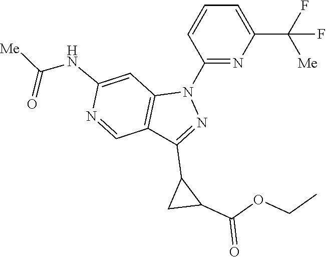 [SMILES: CCOC(=O)C1CC1c1nn(-c2cccc(C(C)(F)F)n2)c2cc(NC(C)=O)ncc12] | LCMS m/z = 430 [M + H]⁺ 1H NMR (400 MHz, CDCl₃) d 12.41 (br s, 1H), 9.90 (s, 1H), 8.82 (s, 1H), 8.10-8.18 (m, 1H), 8.04 (t, J = 7.91 Hz, 1H), 7.68 (d, J = 7.53 Hz, 1H), 3.88-4.04 (m, 2H), 2.68- 2.77 (m, 1H), 2.34-2.42 (m, 4H), 2.29 (t, J = 18.95 Hz, 3H), 2.00-2.10 (m, 1H), 1.69 (dt, J = 5.27, 8.41 Hz, 1H), 1.10 (t, J = 7.15 Hz, 3H). |
| | ethyl 2-(6-acetamido-1-(6-(1,1- | |
| | difluoroethyl)pyridin-2-yl)-1H- | |
| | pyrazolo[4,3-c]pyridin-3-yl)cyclopropane- | |
| | 1-carboxylate | |
| 870 | 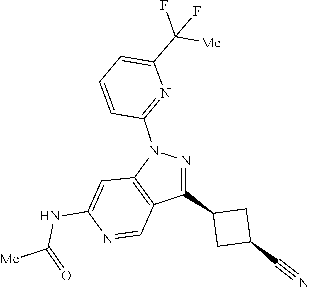 [SMILES: CC(=O)Nc1cc2c(cn1)c([C@H]1C[C@@H](C#N)C1)nn2-c1cccc(C(C)(F)F)n1] | LCMS m/z = 397 [M + H]⁺ 1H NMR (500 MHz, CDCl₃) δ 9.45 (s, 1H), 8.74 (d, J = 0.92 Hz, 1H), 8.10 (d, J = 8.39 Hz, 2H), 7.98 (t, J = 8.01 Hz, 1H), 7.59 (d, J = 7.48 Hz, 1H), 4.25-4.35 (m, 1H), 3.62-3.73 (m, 1H), 2.57-2.66 (m, 3H), 2.46-2.55 (m, 1H), 2.27-2.39 (m, 6H); |
| | N-(3-((1s,3s)-3-cyanocyclobutyl)-1-(6- | |
| | (1,1-difluoroethyl)pyridin-2-yl)-1H- | |
| | pyrazolo[4,3-c]pyridin-6-yl)acetamide | |
| 871 | 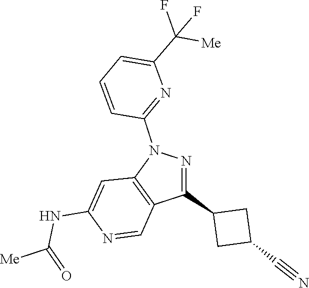 [SMILES: CC(=O)Nc1cc2c(cn1)c([C@H]1C[C@H](C#N)C1)nn2-c1cccc(C(C)(F)F)n1] | LCMS m/z = 397 [M + H]⁺ 1H NMR (500 MHz, CDCl₃) d 9.44 (s, 1H), 8.65 (d, J = 0.76 Hz, 1H), 8.06- 8.16 (m, 2H), 7.97 (t, J = 7.93 Hz, 1H), 7.58 (d, J = 7.48 Hz, 1H), 4.24 (t, J = 7.78 Hz, 1H), 3.43 (t, J = 7.55 Hz, 1H), 2.97 (t, J = 7.71 Hz, 4H), 2.33 (t, J = 19.07 Hz, 3H), 2.28 (s, 3H). |
| | N-(3-((1r,3r)-3-cyanocyclobutyl)-1-(6- | |
| | (1,1-difluoroethyl)pyridin-2-y1)-1H- | |
| | pyrazolo[4,3-c]pyridin-6-yl)acetamide | |
| 872 | 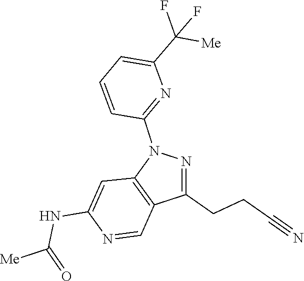 [SMILES: CC(=O)Nc1cc2c(cn1)c(CCC#N)nn2-c1cccc(C(C)(F)F)n1] | LCMS m/z = 371 [M + H]+ 1H NMR (500 MHz, DMSO-d₆) δ 10.78 (s, 1H), 9.37 (d, J = 0.76 Hz, 1H), 9.06 (d, J = 1.07 Hz, 1H), 8.17- 8.25 (m, 1H), 8.10 (d, J = 8.24 Hz, 1H), 7.64 (dd, J = 0.76, 7.63 Hz, 1H), 3.12 (t, J = 7.17 Hz, 2H), 2.27 (t, J = 19.45 Hz, 3H), 2.11-2.22 (m, 3H). |
| | N-(3-(2-cyanoethyl)-1-(6-(1,1- | |
| | difluoroethyl)pyridin-2-yl)-1H- | |
| | pyrazolo[4,3-c]pyridin-6-yl)acetamide | |
| 873 | 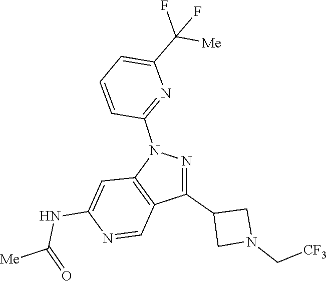 [SMILES: CC(=O)Nc1cc2c(cn1)c(C1CN(CC(F)(F)F)C1)nn2-c1cccc(C(C)(F)F)n1] | LCMS m/z = 455 [M + H]⁺ |
| | N-(1-(6-(1,1-difluoroethyl)pyridin-2-yl)- | |
| | 3-(1-(2,2,2-trifluoroethyl)azetidin-3-yl)- | |
| | 1H-pyrazolo[4,3-c]pyridin-6-yl)acetamide | |
| 874 | 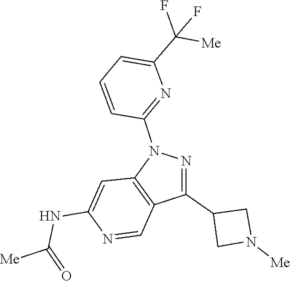 [SMILES: CC(=O)Nc1cc2c(cn1)c(C1CN(C)C1)nn2-c1cccc(C(C)(F)F)n1] | LCMS m/z = 387 [M + H]⁺ |
| | N-(1-(6-(1,1-difluoroethyl)pyridin-2-y1)- | |
| | 3-(1-methylazetidin-3-y1)-1H- | |
| | pyrazolo[4,3-c]pyridin-6-yl)acetamide | |
| 875 | 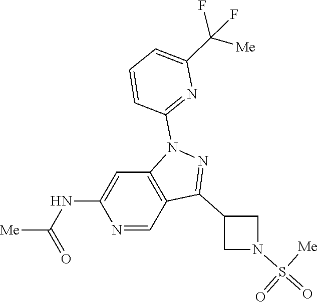 [SMILES: CC(=O)Nc1cc2c(cn1)c(C1CN(S(C)(=O)=O)C1)nn2-c1cccc(C(C)(F)F)n1] | LCMS m/z = 451 [M + H]⁺ 1H NMR (500 MHz, DMSO-d₆) δ 10.80 (s, 1H), 9.39 (s, 1H), 9.01 (s, 1H), 8.13-8.25 (m, 2H), 7.65 (dd, J = 1.83, 6.71 Hz, 1H), 4.37- 4.48 (m, 3H), 4.29-4.35 (m, 2H), 3.05-3.17 (m, 3H), 2.27 (t, J = 19.53 Hz, 3H), 2.10-2.20 (m, 3H). |
| | N-(1-(6-(1,1-difluoroethyl)pyridin-2-yl)- | |
| | 3-(1-(methylsulfonyl)azetidin-3-yl)-1H- | |
| | pyrazolo[4,3-c]pyridin-6-yl)acetamide | |
| 876 | 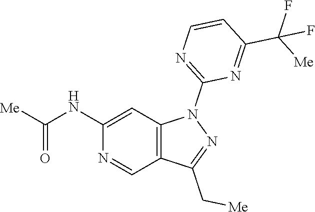 [SMILES: CCc1nn(-c2nccc(C(C)(F)F)n2)c2cc(NC(C)=O)ncc12] | LCMS m/z = 363 [M + H]⁺ |
| | methyl (1-(4-(1,1-difluoroethyl)pyrimidin- | |
| | 2-yl)-3-ethyl-1H-pyrazolo[4,3-c]pyridin- | |
| | 6-yl)carbamate | |
| 877 | 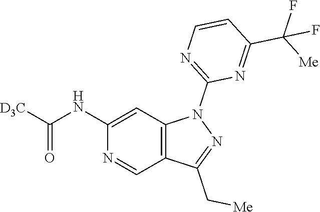 [SMILES: [2H]C([2H])([2H])C(=O)Nc1cc2c(cn1)c(CC)nn2-c1nccc(C(C)(F)F)n1] | LCMS m/z = 350 [M + H]⁺ 1H NMR (400 MHz, CDCl₃) δ 9.47 (s, 1H), 9.03 (d, J = 5.02 Hz, 1H), 8.77 (d, J = 1.00 Hz, 1H), 8.68 (br s, 1H), 7.54 (d, J = 5.02 Hz, 1H), 3.16 (q, J = 7.53 Hz, 2H), 2.31 (t, J = 19.07 Hz, 3H), 1.50 (t, J = 7.53 Hz, 3H). |
| | N-(1-(4-(1,1-difluoroethyl)pyrimidin-2- | |
| | yl)-3-ethyl-1H-pyrazolo[4,3-c]pyridin-6- | |
| | yl)acetamide-2,2,2-d3 | |
| 878 | 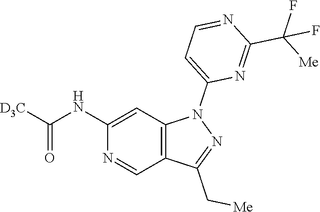 [SMILES: [2H]C([2H])([2H])C(=O)Nc1cc2c(cn1)c(CC)nn2-c1ccnc(C(C)(F)F)n1] | LCMS m/z = 350 [M + H]⁺ 1H NMR (400 MHz, CDCl₃) δ 9.86 (br s, 1H), 9.79 (s, 1H), 8.92 (d, J = 5.52 Hz, 1H), 8.74 (d, J = 1.00 Hz, 1H), 8.03 (d, J = 5.77 Hz, 1H), 3.06-3.15 (m, 2H), 2.30 (t, J = 18.70 Hz, 3H), 1.51 (t, J = 7.53 Hz, 3H). |
| | N-(1-(2-(1,1-difluoroethyl)pyrimidin-4- | |
| | y1)-3-ethyl-1H-pyrazolo[4,3-c]pyridin-6- | |
| | yl)acetamide-2,2,2-d3 | |
| 879 | 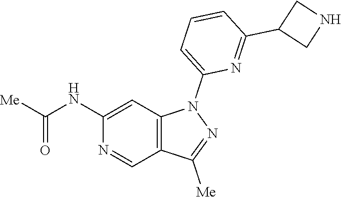 [SMILES: CC(=O)Nc1cc2c(cn1)c(C)nn2-c1cccc(C2CNC2)n1] | LCMS m/z = 323 [M + H]⁺ 1H NMR (DMSO-d₆, 500 MHz) δ 10.93 (s, 1H), 9.52 (s, 1H), 8.97 (d, 1H, J = 0.9 Hz), 7.99 (dd, 1H, J = 7.6, 8.2 Hz), 7.87 (d, 1H, J = 7.8 Hz), 7.25 (d, 1H, J = 7.2 Hz), 4.5-4.5 (m, 2H), 4.3-4.4 (m, 3H), 2.6-2.7 (m, 3H), 2.18 (s, 3H). |
| | N-(1-(6-(azetidin-3-y1)pyridin-2-y1)-3- | |
| | methyl-1H-pyrazolo[4,3-c]pyridin-6- | |
| | yl)acetamide | |
| 880 | 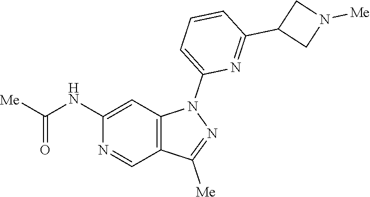 [SMILES: CC(=O)Nc1cc2c(cn1)c(C)nn2-c1cccc(C2CN(C)C2)n1] | LCMS m/z = 337 [M + H]⁺ |
| | N-(3-methyl-1-(6-(1-methylazetidin-3- | |
| | yl)pyridin-2-yl)-1H-pyrazolo[4,3- | |
| | c]pyridin-6-yl)acetamide | |
| 881 | 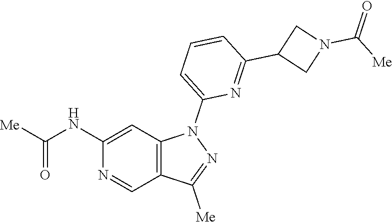 [SMILES: CC(=O)Nc1cc2c(cn1)c(C)nn2-c1cccc(C2CN(C(C)=O)C2)n1] | LCMS m/z = 365 [M + H]⁺ |
| | N-(1-(6-(1-acetylazetidin-3-yl)pyridin-2- | |
| | yl)-3-methyl-1H-pyrazolo[4,3-c]pyridin- | |
| | 6-yl)acetamide | |
| 882 | 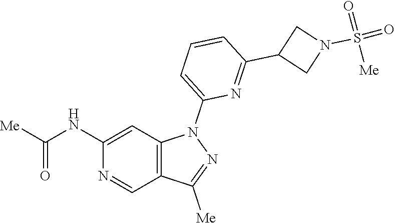 [SMILES: CC(=O)Nc1cc2c(cn1)c(C)nn2-c1cccc(C2CN(S(C)(=O)=O)C2)n1] | LCMS m/z = 401 [M + H]⁺ 1H NMR (DMSO-d₆, 600 MHz) δ 10.67 (br d, 1H, J = 6.5 Hz), 9.29 (s, 1H), 8.92 (s, 1H), 7.98 (t, 1H, J = 8.0 Hz), 7.81 (d, 1H, J = 8.7 Hz), 7.29 (d, 1H, J = 8.0 Hz), 4.3-4.3 (m, 2H), 4.28 (d, 2H, J = 5.1 Hz), 4.17 (q, 1H, J = 8.0 Hz), 3.00 (s, 3H), 2.64 (s, 3H), 2.15 (s, 3H). |
| | N-(3-methyl-1-(6-(1- | |
| | (methylsulfonyl)azetidin-3-yl)pyridin-2- | |
| | yl)-1H-pyrazolo[4,3-c]pyridin-6- | |
| | yl)acetamide | |
| 883 | 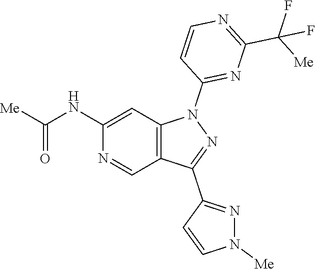 [SMILES: CC(=O)Nc1cc2c(cn1)c(-c1ccn(C)n1)nn2-c1ccnc(C(C)(F)F)n1] | LCMS m/z = 399.3 [M + H]+ |
| | N-(1-(2-(1,1-difluoroethyl)pyrimidin-4- | |
| | y1)-3-(1-methyl-1H-pyrazol-3-y1)-1H- | |
| | pyrazolo[4,3-c]pyridin-6-yl)acetamide | |
| 884 | 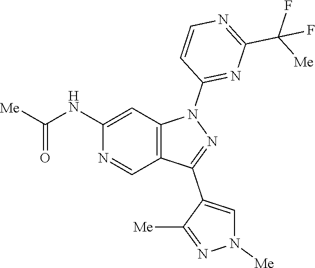 [SMILES: CC(=O)Nc1cc2c(cn1)c(-c1cn(C)nc1C)nn2-c1ccnc(C(C)(F)F)n1] | LCMS m/z = 413.3 [M + H]+ 1H NMR (600 MHz, DMSO-d₆) (ppm) 10.86 (s, 1H), 9.51-9.45 (m, 1H), 9.23-9.17 (m, 1H), 9.02-8.98 (m, 1H), 8.78-8.74 (m, 1H), 8.08- 8.02 (m, 1H), 3.91 (s, 3H), 2.33- 2.22 (m, 3H), 2.18 (s, 3H) |
| | N-(1-(2-(1,1-difluoroethyl)pyrimidin-4- | |
| | yl)-3-(1,3-dimethyl-1H-pyrazol-4-yl)-1H- | |
| | pyrazolo[4,3-c]pyridin-6-yl)acetamide | |
| 885 | 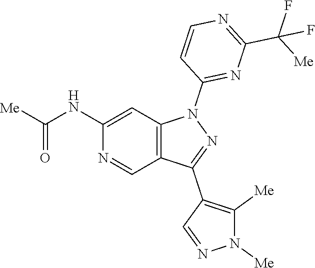 [SMILES: CC(=O)Nc1cc2c(cn1)c(-c1cnn(C)c1C)nn2-c1ccnc(C(C)(F)F)n1] | LCMS m/z = 413.3 [M + H]+ |
| | N-(1-(2-(1,1-difluoroethyl)pyrimidin-4- | |
| | yl)-3-(1,5-dimethyl-1H-pyrazol-4-y1)-1H- | |
| | pyrazolo[4,3-c]pyridin-6-yl)acetamide | |
| 886 | 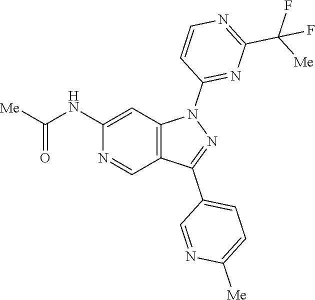 [SMILES: CC(=O)Nc1cc2c(cn1)c(-c1ccc(C)nc1)nn2-c1ccnc(C(C)(F)F)n1] | LCMS m/z = 410.3 [M + H]+ 1H NMR (600 MHz, DMSO-d₆) (ppm) 10.93 (s, 1H), 9.59-9.48 (m, 1H), 9.35 (s, 1H), 9.31-9.26 (m, 1H), 9.08-9.04 (m, 1H), 8.58-8.49 (m, 1H), 8.28-8.19 (m, 1H), 7.59- 7.51 (m, 1H), 2.28 (t, J = 18.9 Hz, 3H), 2.19 (s, 3H) |
| | N-(1-(2-(1,1-difluoroethyl)pyrimidin-4- | |
| | y1)-3-(6-methylpyridin-3-y1)-1H- | |
| | pyrazolo[4,3-c]pyridin-6-yl)acetamide | |
| | trifluoroacetate | |
| 887 | 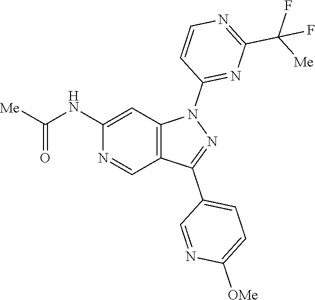 [SMILES: COc1ccc(-c2nn(-c3ccnc(C(C)(F)F)n3)c3cc(NC(C)=O)ncc23)cn1] | LCMS m/z = 426.3 [M + H]+ |
| | N-(1-(2-(1,1-difluoroethyl)pyrimidin-4- | |
| | yl)-3-(6-methoxypyridin-3-yl)-1H- | |
| | pyrazolo[4,3-c]pyridin-6-yl)acetamide | |
| 888 | 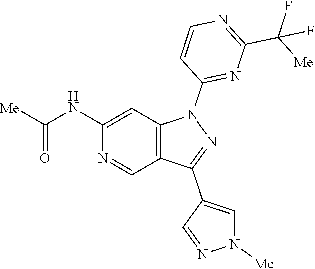 [SMILES: CC(=O)Nc1cc2c(cn1)c(-c1cnn(C)c1)nn2-c1ccnc(C(C)(F)F)n1] | LCMS m/z = 399.3 [M + H]+ |
| | N-(1-(2-(1,1-difluoroethyl)pyrimidin-4- | |
| | y1)-3-(1-methyl-1H-pyrazol-4-yl)-1H- | |
| | pyrazolo[4,3-c]pyridin-6-yl)acetamide | |
| 889 | 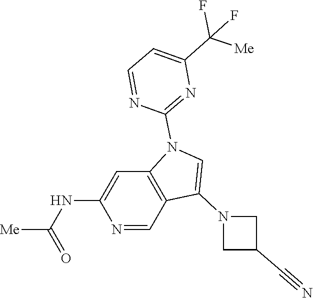 [SMILES: CC(=O)Nc1cc2c(cn1)c(N1CC(C#N)C1)cn2-c1nccc(C(C)(F)F)n1] | LCMS m/z = 398 [M + H]⁺ 1H NMR (DMSO-d₆, 600 MHz) δ 10.46 (br s, 1H), 9.3-9.4 (m, 1H), 9.03 (d, 1H, J = 5.1 Hz), 8.58 (s, 1H), 7.55 (d, 1H, J = 5.1 Hz), 7.47 (s, 1H), 4.28 (t, 2H, J = 7.6 Hz), 4.18 (t, 2H, J = 6.2 Hz), 3.90-3.92 (m, 1H), 2.20-2.30 (m, 3H), 2.10- 2.20 (m, 3H). |
| | N-(3-(3-cyanoazetidin-1-yl)-1-(4-(1,1- | |
| | difluoroethyl)pyrimidin-2-yl)-1H- | |
| | pyrrolo[3,2-c]pyridin-6-yl)acetamide | |
| 890 | 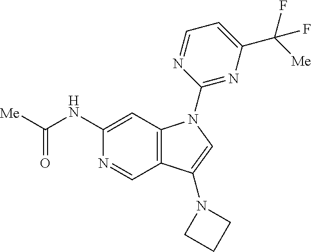 [SMILES: CC(=O)Nc1cc2c(cn1)c(N1CCC1)cn2-c1nccc(C(C)(F)F)n1] | LCMS m/z = 373 [M + H]⁺ 1H NMR (MeOD-d₄, 400 MHz) δ 9.37 (s, 1H), 8.92 (d, 1H, J = 5.0 Hz), 8.49 (d, 1H, J = 1.0 Hz), 7.49 (s, 1H), 7.45 (d, 1H, J = 5.0 Hz), 4.00-4.10 (m, 4H), 2.40-2.50 (m, 2H), 2.10-2.20 (m, 6H). |
| | N-(3-(azetidin-1-yl)-1-(4-(1,1- | |
| | difluoroethyl)pyrimidin-2-yl)-1H- | |
| | pyrrolo[3,2-c]pyridin-6-yl)acetamide | |
| 891 | 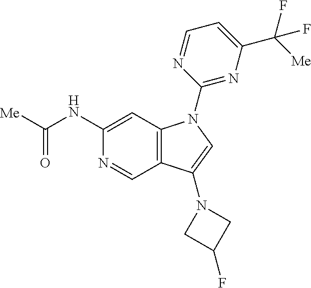 [SMILES: CC(=O)Nc1cc2c(cn1)c(N1CC(F)C1)cn2-c1nccc(C(C)(F)F)n1] | LCMS m/z = 391 [M + H]⁺ 1H NMR (MeOD-d₄, 400 MHz) δ 9.37 (s, 1H), 8.92 (d, 1H, J = 5.0 Hz), 8.49 (d, 1H, J = 0.8 Hz), 7.53 (s, 1H), 7.46 (d, 1H, J = 5.0 Hz), 5.40- 5.60 (m, 1H), 4.30-4.40 (m, 2H), 4.00-4.20 (m, 2H), 2.10-2.20 (m, 6H). |
| | N-(1-(4-(1,1-difluoroethyl)pyrimidin-2- | |
| | yl)-3-(3-fluoroazetidin-1-y1)-1H- | |
| | pyrrolo[3,2-c]pyridin-6-yl)acetamide | |
| 892 | 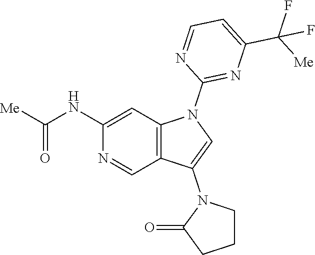 [SMILES: CC(=O)Nc1cc2c(cn1)c(N1CCCC1=O)cn2-c1nccc(C(C)(F)F)n1] | LCMS m/z = 401 [M + H]⁺ 1H NMR (MeOD-d₄, 400 MHz) δ 9.47 (s, 1H), 8.99 (d, 1H, J = 5.0 Hz), 8.83 (d, 1H, J = 0.8 Hz), 8.45 (s, 1H), 7.50-7.60 (m, 1H), 4.11 (t, 2H, J = 7.2 Hz), 2.60-2.70 (m, 2H), 2.30-2.40 (m, 2H), 2.10-2.30 (m, 6H). |
| | N-(1-(4-(1,1-difluoroethyl)pyrimidin-2- | |
| | yl)-3-(2-oxopyrrolidin-1-yl)-1H- | |
| | pyrrolo[3,2-c]pyridin-6-yl)acetamide | |
| 893 | 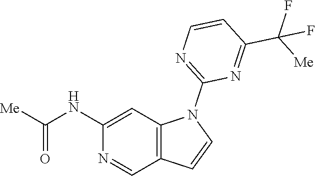 [SMILES: CC(=O)Nc1cc2c(ccn2-c2nccc(C(C)(F)F)n2)cn1] | LCMS m/z = 318 [M + H]⁺ |
| | N-(1-(4-(1,1-difluoroethyl)pyrimidin-2- | |
| | yl)-1H-pyrrolo[3,2-c]pyridin-6- | |
| | yl)acetamide | |
| 894 | 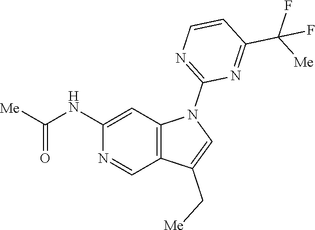 [SMILES: CCc1cn(-c2nccc(C(C)(F)F)n2)c2cc(NC(C)=O)ncc12] | LCMS m/z = 346 [M + H]⁺ 1H NMR (CDCl₃, 400 MHz) δ 9.48 (s, 1H), 8.88 (d, 1H, J = 4.8 Hz), 8.53 (s, 1H), 8.30 (br s, 1H), 8.01 (s, 1H), 7.40-7.42 (m, 1H), 2.83 (dq, 2H, J = 1.0, 7.4 Hz), 2.20-2.30 (m, 6H), 1.41 (t, 3H, J = 7.5 Hz). |
| | N-(1-(4-(1,1-difluoroethyl)pyrimidin-2- | |
| | y1)-3-ethyl-1H-pyrrolo[3,2-c]pyridin-6- | |
| | yl)acetamide | |
| 895 | 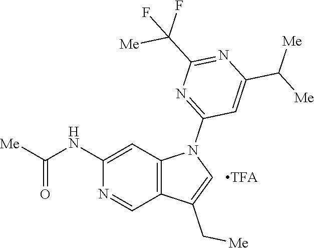 [SMILES: CCc1cn(-c2cc(C(C)C)nc(C(C)(F)F)n2)c2cc(NC(C)=O)ncc12] | LCMS m/z = 388.1 [M + H]⁺ 1H NMR (600 MHz, DMSO-d₆) (ppm) 10.83 (s, 1 H), 9.35 (s, 1 H), 9.03 (s, 1 H), 7.98 (s, 1 H), 2.54- 2.51 (m, 1 H), 2.29-2.19 (m, 3 H), 2.16 (s, 3 H), 1.31-1.12 (m, 4 H) |
| | N-(1-(2-(1,1-difluoroethyl)-6- | |
| | isopropylpyrimidin-4-yl)-3-ethyl-1H- | |
| | pyrrolo[3,2-c]pyridin-6-yl)acetamide | |
| 896 | 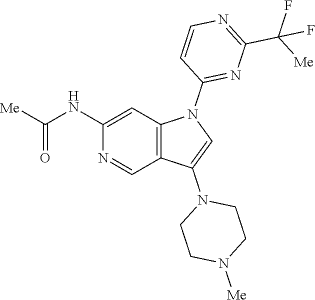 [SMILES: CC(=O)Nc1cc2c(cn1)c(N1CCN(C)CC1)cn2-c1ccnc(C(C)(F)F)n1] | LCMS m/z = 416 [M + H]⁺ 1H NMR (MeOD-d₄, 400 MHz) δ 8.9- 9.0 (m, 2H), 8.85 (s, 1H), 7.96 (s, 1H), 7.90-7.92 (m, 1H), 3.81 (br d, 2H, J = 10.8 Hz), 3.70 (br d, 2H, J = 10.3 Hz), 3.47 (br s, 2H), 3.20- 3.30 (m, 2H), 3.03 (s, 3H), 2.33 (s, 3H), 2.10-2.20 (m, 3H). |
| | N-(1-(2-(1,1-difluoroethyl)pyrimidin-4- | |
| | yl)-3-(4-methylpiperazin-1-yl)-1H- | |
| | pyrrolo[3,2-c]pyridin-6-yl)acetamide | |
| 897 | 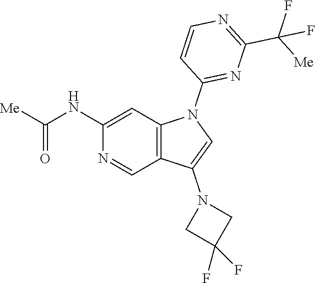 [SMILES: CC(=O)Nc1cc2c(cn1)c(N1CC(F)(F)C1)cn2-c1ccnc(C(C)(F)F)n1] | LCMS m/z = 409 [M + H]⁺ |
| | N-(3-(3,3-difluoroazetidin-1-y1)-1-(2-(1,1- | |
| | difluoroethyl)pyrimidin-4-yl)-1H- | |
| | pyrrolo[3,2-c]pyridin-6-yl)acetamide | |
| 898 | 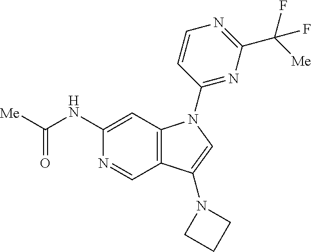 [SMILES: CC(=O)Nc1cc2c(cn1)c(N1CCC1)cn2-c1ccnc(C(C)(F)F)n1] | LCMS m/z = 373 [M + H]⁺ 1H NMR (MeOD-d₄, 400 MHz) δ 9.37 (s, 1H), 8.78 (d, 1H, J = 5.8 Hz), 8.51 (d, 1H, J = 0.8 Hz), 7.65 (d, 1H, J = 5.8 Hz), 7.17 (s, 1H), 4.07 (t, 4H, J = 7.2 Hz), 2.47 (quin, 2H, J = 7.3 Hz), 2.20-2.30 (m, 6H) |
| | N-(3-(azetidin-1-y1)-1-(2-(1,1- | |
| | difluoroethyl)pyrimidin-4-yl)-1H- | |
| | pyrrolo[3,2-c]pyridin-6-yl)acetamide | |
| 899 | 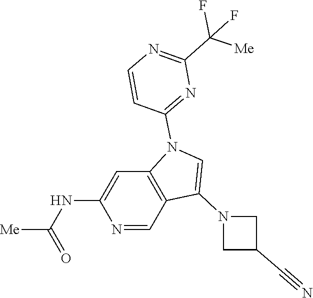 [SMILES: CC(=O)Nc1cc2c(cn1)c(N1CC(C#N)C1)cn2-c1ccnc(C(C)(F)F)n1] | LCMS m/z = 398 [M + H]⁺ 1H NMR (MeOD-d₄, 400 MHz) δ 9.39 (s, 1H), 8.80 (d, 1H, J = 6.0 Hz), 8.52 (d, 1H, J = 0.8 Hz), 7.68 (d, 1H, J = 6.0 Hz), 7.31 (s, 1H), 4.35 (dd, 2H, J = 7.4, 8.2 Hz), 4.20 (t, 2H, J = 6.7 Hz), 3.80-3.90 (m, 1H), 2.20-2.30 (m, 6H). |
| | N-(3-(3-cyanoazetidin-1-yl)-1-(2-(1,1- | |
| | difluoroethyl)pyrimidin-4-yl)-1H- | |
| | pyrrolo[3,2-c]pyridin-6-yl)acetamide | |
| 900 | 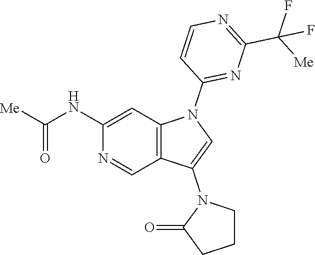 [SMILES: CC(=O)Nc1cc2c(cn1)c(N1CCCC1=O)cn2-c1ccnc(C(C)(F)F)n1] | LCMS m/z = 401 [M + H]⁺ 1H NMR (CDCl₃, 400 MHz) δ 9.30- 9.50 (m, 1H), 8.90-8.92 (m, 1H), 8.84 (s, 1H), 8.10-8.30 (m, 2H), 7.50 (d, 1H, J = 5.8 Hz), 4.17 (t, 2H, J = 7.0 Hz), 2.60-2.70 (m, 2H), 2.30-2.40 (m, 2H), 2.20-2.30 (m, 6H). |
| | N-(1-(2-(1,1-difluoroethyl)pyrimidin-4- | |
| | yl)-3-(2-oxopyrrolidin-1-yl)-1H- | |
| | pyrrolo[3,2-c]pyridin-6-yl)acetamide | |
| 901 | 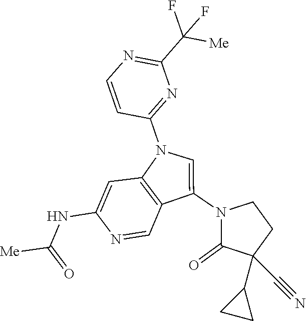 [SMILES: CC(=O)Nc1cc2c(cn1)c(N1CCC(C#N)(C3CC3)C1=O)cn2-c1ccnc(C(C)(F)F)n1] | LCMS m/z = 466 [M + H]⁺). 1H NMR (CDCl₃, 400 MHz) δ 9.40- 9.50 (m, 1H), 9.10-9.30 (m, 1H), 8.96 (d, 1H, J = 5.5 Hz), 8.80-8.90 (m, 1H), 8.20-8.30 (m, 1H), 7.50- 7.60 (m, 1H), 5.80-6.00 (m, 1H), 5.30-5.50 (m, 2H), 4.20-4.30 (m, 1H), 4.11 (dt, 1H, J = 4.6, 8.8 Hz), 2.92 (dd, 1H, J = 7.0, 14.1 Hz), 2.70-2.80 (m, 1H), 2.68 (dd, 1H, J = 7.7, 13.9 Hz), 2.50-2.60 (m, 1H), 2.32 (s, 3H), 2.24 (t, 3H, J = 18.7 Hz). |
| | N-(3-(3-cyano-3-cyclopropyl-2- | |
| | oxopyrrolidin-1-yl)-1-(2-(1,1- | |
| | difluoroethyl)pyrimidin-4-yl)-1H- | |
| | pyrrolo[3,2-c]pyridin-6-yl)acetamide | |
| 902 | 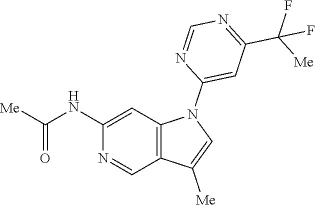 [SMILES: CC(=O)Nc1cc2c(cn1)c(C)cn2-c1cc(C(C)(F)F)ncn1] | LCMS m/z = 332 [M + H]⁺ |
| | N-(1-(6-(1,1-difluoroethyl)pyrimidin-4- | |
| | yl)-3-methyl-1H-pyrrolo[3,2-c]pyridin-6- | |
| | y1)acetamide | |
| 903 | 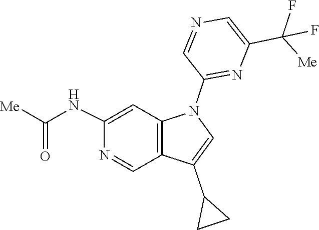 [SMILES: CC(=O)Nc1cc2c(cn1)c(C1CC1)cn2-c1cncc(C(C)(F)F)n1] | LCMS m/z = 358 [M + H]⁺ 1H NMR (400 MHz, CDCl₃) δ 9.20 (s, 1H), 8.64 (d, 1H, J = 0.8 Hz), 8.31 (br s, 1H), 7.43 (d, 1H, J = 1.0 Hz), 7.10 (s, 1H), 4.30-4.40 (m, 1H), 4.10-4.20 (m, 2H), 3.90-4.10 (m, 1H), 3.70-3.90 (m, 1H), 2.50-2.70 (m, 4H), 2.40-2.50 (m, 1H), 2.26 (s, 3H), 1.90-2.00 (m, 1H), 1.00- 1.10 (m, 2H), 0.70-0.80 (m, 2H). |
| | N-(3-cyclopropyl-1-(6-(1,1- | |
| | difluoroethyl)pyrazin-2-y1)-1H- | |
| | pyrrolo[3,2-c]pyridin-6-y1)acetamide | |
| 904 | 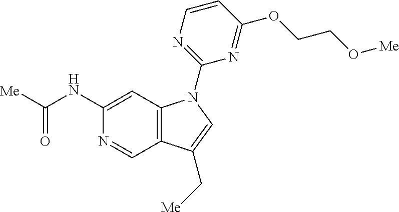 [SMILES: CCc1cn(-c2nccc(OCCOC)n2)c2cc(NC(C)=O)ncc12] | LCMS m/z = 357 [M + H]⁺ 1H NMR (400 MHz, CDCl₃) δ 9.50 (s, 1H), 9.14 (br d, J = 16.31 Hz, 1H), 8.74 (d, J = 0.75 Hz, 1H), 8.55 (d, J = 5.52 Hz, 1H), 6.72 (d, J = 5.77 Hz, 1H), 4.88-4.94 (m, 2H), 3.90- 3.95 (m, 2H), 3.49 (s, 3H), 3.15 (q, J = 7.78 Hz, 2H), 2.29 (s, 3H), 1.49 (t, J = 7.65 Hz, 3H). |
| | N-(3-ethyl-1-(4-(2- | |
| | methoxyethoxy)pyrimidin-2-yl)-1H- | |
| | pyrrolo[3,2-c]pyridin-6-yl)acetamide | |
| 905 | 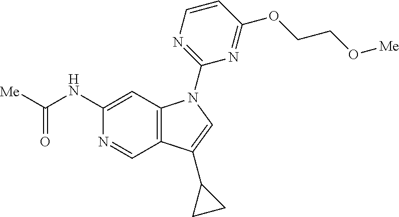 [SMILES: COCCOc1ccnc(-n2cc(C3CC3)c3cnc(NC(C)=O)cc32)n1] | LCMS m/z = 368 [M + H]⁺ 1H NMR (MeOD-d₄, 400 MHz) δ 9.30-9.40 (m, 1H), 8.62 (d, 1H, J = 0.8 Hz), 8.42 (d, 1H, J = 5.5 Hz), 7.90 (d, 1H, J = 1.0 Hz), 6.65 (d, 1H, J = 5.5 Hz), 4.80-4.82 (m, 2H), 3.80-3.90 (m, 2H), 3.44 (s, 3H), 2.21 (s, 3H), 2.00-2.10 (m, 1H), 1.00-1.02 (m, 2H), 0.70-0.80 (m, 2H). |
| | N-(3-cyclopropyl-1-(4-(2- | |
| | methoxyethoxy)pyrimidin-2-yl)-1H- | |
| | pyrrolo[3,2-c]pyridin-6-yl)acetamide | |
| 906 | 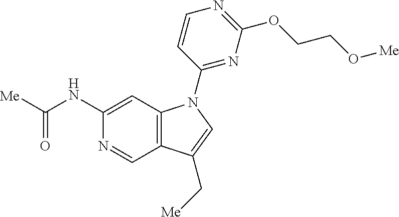 [SMILES: CCc1cn(-c2ccnc(OCCOC)n2)c2cc(NC(C)=O)ncc12] | LCMS m/z = 357 [M + H]⁺ 1H NMR (400 MHz, CDCl₃) δ 9.64 (s, 1H), 9.48 (br s, 1H), 8.71 (d, J = 0.75 Hz, 1H), 8.59 (d, J = 5.52 Hz, 1H), 7.57 (d, J = 5.52 Hz, 1H), 4.79-4.85 (m, 2H), 3.92-3.97 (m, 2H), 3.49 (s, 3H), 3.08 (q, J = 7.53 Hz, 2H), 2.31 (s, 3H), 1.50 (t, J = 7.65 Hz, 3H). |
| | N-(3-ethyl-1-(2-(2- | |
| | methoxyethoxy)pyrimidin-4-yl)-1H- | |
| | pyrrolo[3,2-c]pyridin-6-yl)acetamide | |
| 907 | 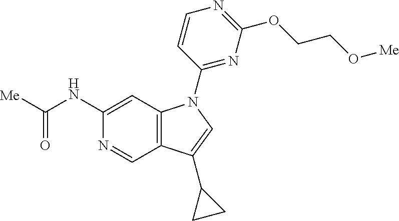 [SMILES: COCCOc1nccc(-n2cc(C3CC3)c3cnc(NC(C)=O)cc32)n1] | LCMS m/z = 368 [M + H]⁺ 1H NMR (MeOD-d₄, 400 MHz) δ 9.34 (s, 1H), 8.66 (d, 1H, J = 0.8 Hz), 8.50 (d, 1H, J = 5.8 Hz), 7.65 (d, 1H, J = 0.8 Hz), 7.28 (d, 1H, J = 5.8 Hz), 4.70-4.80 (m, 2H), 3.80-3.90 (m, 2H), 3.44 (s, 3H), 2.21 (s, 3H), 2.00-2.10 (m, 1H), 1.00-1.10 (m, 2H), 0.80-0.82 (m, 2H). |
| | N-(3-cyclopropyl-1-(2-(2- | |
| | methoxyethoxy)pyrimidin-4-yl)-1H- | |
| | pyrrolo[3,2-c]pyridin-6-yl)acetamide | |
| 908 | 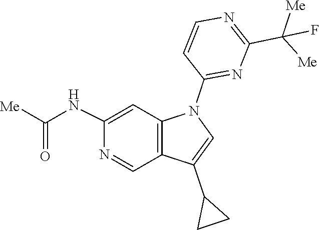 [SMILES: CC(=O)Nc1cc2c(cn1)c(C1CC1)cn2-c1ccnc(C(C)(C)F)n1] | LCMS m/z = 354 [M + H]⁺ 1H NMR (CDCl₃, 400 MHz) δ 9.20- 9.40 (m, 1H), 8.80 (d, 1H, J = 5.8 Hz), 8.65 (d, 1H, J = 0.8 Hz), 8.10- 8.30 (m, 1H), 7.42 (d, 1H, J = 1.0 Hz), 7.29 (d, 1H, J = 5.8 Hz), 2.27 (s, 3H), 1.97 (s, 3H), 1.92 (s, 3H), 1.00-1.10 (m, 2H), 0.70-0.80 (m, 2H). |
| | N-(3-cyclopropyl-1-(2-(2-fluoropropan-2- | |
| | yl)pyrimidin-4-yl)-1H-pyrrolo[3,2- | |
| | c]pyridin-6-yl)acetamide | |
| 909 | 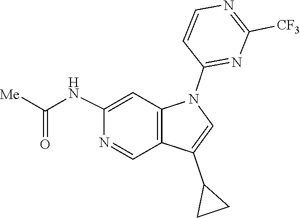 [SMILES: CC(=O)Nc1cc2c(cn1)c(C1CC1)cn2-c1ccnc(C(F)(F)F)n1] | LCMS m/z = 362 [M + H]⁺ 1H NMR (CDCl₃, 400 MHz) δ 9.10- 9.30 (m, 1H), 8.89 (d, 1H, J = 5.8 Hz), 8.60-8.70 (m, 1H), 8.34 (br s, 1H), 7.60 (d, 1H, J = 5.8 Hz), 7.40- 7.50 (m, 1H), 2.29 (s, 3H), 1.90- 2.00 (m, 1H), 1.00-1.10 (m, 2H), 0.70-0.80 (m, 2H). |
| | N-(3-cyclopropyl-1-(2- | |
| | (trifluoromethyl)pyrimidin-4-y1)-1H- | |
| | pyrrolo[3,2-c]pyridin-6-yl)acetamide | |
| 910 | 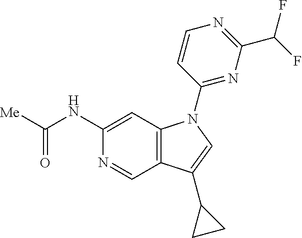 [SMILES: CC(=O)Nc1cc2c(cn1)c(C1CC1)cn2-c1ccnc(C(F)F)n1] | LCMS m/z = 344 [M + H]⁺ 1H NMR (CDCl₃, 400 MHz) δ 9.29 (s, 1H), 8.87 (d, 1H, J = 5.8 Hz), 8.66 (s, 1H), 8.47 (br s, 1H), 7.53 (dd, 2H, J = 2.5, 3.3 Hz), 6.73 (t, 1H, J = 54.5 Hz), 2.29 (s, 3H), 1.90-2.00 (m, 1H), 1.00-1.10 (m, 2H), 0.70- 0.80 (m, 2H). |
| | N-(3-cyclopropyl-1-(2- | |
| | (difluoromethyl)pyrimidin-4-y1)-1H- | |
| | pyrrolo[3,2-c]pyridin-6-yl)acetamide | |
| 911 | 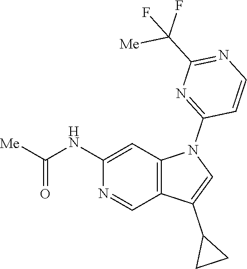 [SMILES: CC(=O)Nc1cc2c(cn1)c(C1CC1)cn2-c1ccnc(C(C)(F)F)n1] | LCMS m/z = 346 [M + H]⁺ 1H NMR (CDCl₃, 400 MHz) δ 9.28 (s, 1H), 8.86 (d, 1H, J = 5.8 Hz), 8.56 (d, 1H, J = 0.8 Hz), 8.23 (br s, 1H), 7.55 (s, 1H), 7.40-7.50 (m, 1H), 2.83 (dq, 2H, J = 1.1, 7.5 Hz), 2.20- 2.30 (m, 6H), 1.41 (t, 3H, J = 7.5 Hz). |
| | N-(1-(2-(1,1-difluoroethyl)pyrimidin-4- | |
| | yl)-3-ethyl-1H-pyrrolo[3,2-c]pyridin-6- | |
| | yl)acetamide | |
| 912 | 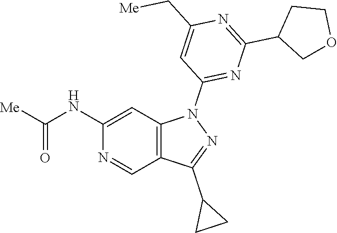 [SMILES: CCc1cc(-n2nc(C3CC3)c3cnc(NC(C)=O)cc32)nc(C2CCOC2)n1] | LCMS m/z = 409 [M + H]⁺ |
| | methyl (3-cyclopropyl-1-(6-ethyl-2- | |
| | (tetrahydrofuran-3-yl)pyrimidin-4-yl)-1H- | |
| | pyrazolo[4,3-c]pyridin-6-yl)carbamate | |
| | trifluoroacetate | |
| 913 | 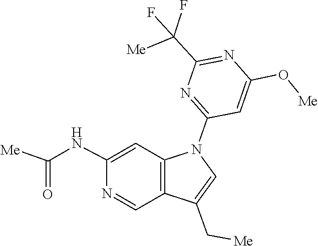 [SMILES: CCc1cn(-c2cc(OC)nc(C(C)(F)F)n2)c2cc(NC(C)=O)ncc12] | LCMS m/z = 376.3 [M + H]⁺ |
| | N-(1-(2-(1,1-difluoroethyl)-6- | |
| | methoxypyrimidin-4-yl)-3-ethyl-1H- | |
| | pyrrolo[3,2-c]pyridin-6-yl)acetamide | |
| 914 | 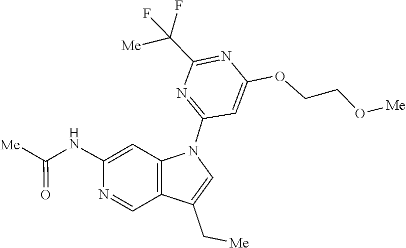 [SMILES: CCc1cn(-c2cc(OCCOC)nc(C(C)(F)F)n2)c2cc(NC(C)=O)ncc12] | LCMS m/z = 420.3 [M + H]⁺ |
| | N-(1-(2-(1,1-difluoroethyl)-6-(2- | |
| | methoxyethoxy)pyrimidin-4-yl)-3-ethyl- | |
| | 1H-pyrrolo[3,2-c]pyridin-6-yl)acetamide | |
| 915 | 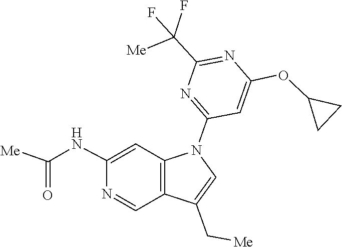 [SMILES: CCc1cn(-c2cc(OC3CC3)nc(C(C)(F)F)n2)c2cc(NC(C)=O)ncc12] | LCMS m/z = 402.3 [M + H]⁺ |
| | N-(1-(6-cyclopropoxy-2-(1,1- | |
| | difluoroethyl)pyrimidin-4-yl)-3-ethyl-1H- | |
| | pyrrolo[3,2-c]pyridin-6-yl)acetamide | |
| 916 | 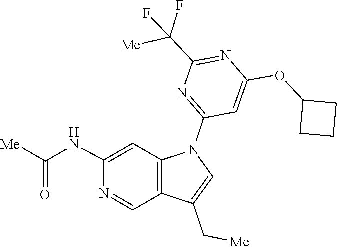 [SMILES: CCc1cn(-c2cc(OC3CCC3)nc(C(C)(F)F)n2)c2cc(NC(C)=O)ncc12] | LCMS m/z = 416.1 [M + H]⁺ |
| | N-(1-(6-cyclobutoxy-2-(1,1- | |
| | difluoroethyl)pyrimidin-4-y1)-3-ethyl-1H- | |
| | pyrrolo[3,2-c]pyridin-6-yl)acetamide | |
| 917 | 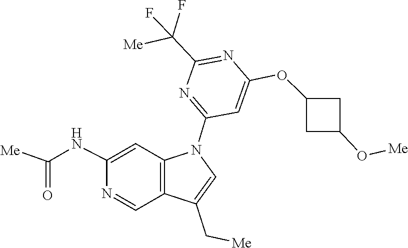 [SMILES: CCc1cn(-c2cc(OC3CC(OC)C3)nc(C(C)(F)F)n2)c2cc(NC(C)=O)ncc12] | LCMS m/z = 446.1 [M + H]⁺ |
| | N-(1-(2-(1,1-difluoroethyl)-6-(3- | |
| | methoxycyclobutoxy)pyrimidin-4-yl)-3- | |
| | ethyl-1H-pyrrolo[3,2-c]pyridin-6- | |
| | yl)acetamide | |
| 918 | 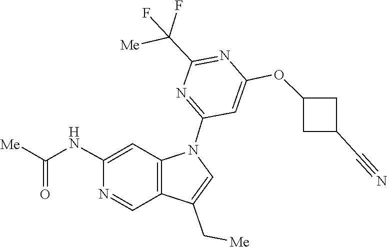 [SMILES: CCc1cn(-c2cc(OC3CC(C#N)C3)nc(C(C)(F)F)n2)c2cc(NC(C)=O)ncc12] | LCMS m/z = 441.3 [M + H]⁺ |
| | N-(1-(6-(3-cyanocyclobutoxy)-2-(1,1- | |
| | difluoroethyl)pyrimidin-4-yl)-3-ethyl-1H- | |
| | pyrrolo[3,2-c]pyridin-6-yl)acetamide | |
| 919 | 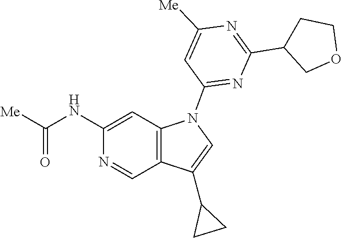 [SMILES: CC(=O)Nc1cc2c(cn1)c(C1CC1)cn2-c1cc(C)nc(C2CCOC2)n1] | LCMS m/z = 378 [M + H]⁺ 1H NMR (400 MHz, CDCl₃) δ 9.20 (s, 1H), 8.64 (d, 1H, J = 0.8 Hz), 8.31 (br s, 1H), 7.43 (d, 1H, J = 1.0 Hz), 7.10 (s, 1H), 4.30-4.40 (m, 1H), 4.10-4.20 (m, 2H), 3.90-4.10 (m, 1H), 3.70-3.90 (m, 1H), 2.50-2.70 (m, 4H), 2.40-2.50 (m, 1H), 2.26 (s, 3H), 1.90-2.00 (m, 1H), 1.00- 1.10 (m, 2H), 0.70-0.80 (m, 2H). |
| | N-(3-cyclopropyl-1-(6-methyl-2- | |
| | (tetrahydrofuran-3-yl)pyrimidin-4-yl)-1H- | |
| | pyrrolo[3,2-c]pyridin-6-yl)acetamide | |
| 920 | 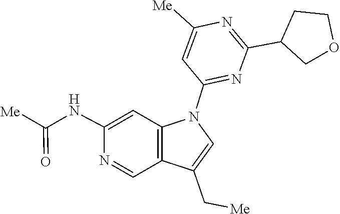 [SMILES: CCc1cn(-c2cc(C)nc(C3CCOC3)n2)c2cc(NC(C)=O)ncc12] | LCMS m/z = 366 [M + H]⁺ 1H NMR (400 MHz, CDCl₃) δ 9.25 (s, 1H), 8.53 (d, 1H, J = 0.8 Hz), 8.40- 8.50 (m, 1H), 7.50-7.60 (m, 1H), 7.13 (s, 1H), 4.30-4.40 (m, 1H), 4.10-4.20 (m, 2H), 4.00-4.10 (m, 1H), 3.70-3.90 (m, 1H), 2.83 (dq, 2H, J = 1.0, 7.5 Hz), 2.50-2.70 (m, 4H), 2.40-2.50 (m, 1H), 2.27 (s, 3H). |
| | N-(3-ethyl-1-(6-methyl-2- | |
| | (tetrahydrofuran-3-yl)pyrimidin-4-yl)-1H- | |
| | pyrrolo[3,2-c]pyridin-6-yl)acetamide | |
| 921 | 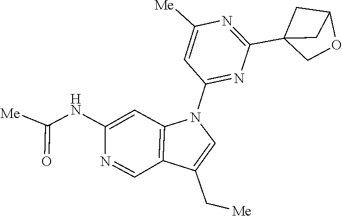 [SMILES: CCc1cn(-c2cc(C)nc(C34COC(C3)C4)n2)c2cc(NC(C)=O)ncc12] | LCMS m/z = 378 [M + H]⁺ |
| | N-(1-(2-(2-oxabicyclo[2.1.1]hexan-4-yl)- | |
| | 6-methylpyrimidin-4-y1)-3-ethyl-1H- | |
| | pyrrolo[3,2-c]pyridin-6-yl)acetamide | |
| 922 | 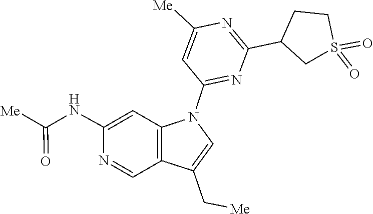 [SMILES: CCc1cn(-c2cc(C)nc(C3CCS(=O)(=O)C3)n2)c2cc(NC(C)=O)ncc12] | LCMS m/z = 414 [M + H]⁺ 1H NMR (400 MHz, CDCl₃) δ 9.31 (s, 1H), 8.54 (s, 2H), 7.50-7.60 (m, 1H), 7.16 (s, 1H), 4.00-4.10 (m, 1H), 3.70-3.80 (m, 1H), 3.60-3.70 (m, 1H), 3.46 (ddd, 1H, J = 4.9, 8.3, 13.1 Hz), 3.25 (td, 1H, J = 8.5, 13.1 Hz), 2.70-2.90 (m, 4H), 2.61 (s, 3H), 2.27 (s, 3H), 1.41 (t, 3H, J = 7.5 Hz). |
| | N-(1-(2-(1,1-dioxidotetrahydrothiophen- | |
| | 3-y1)-6-methylpyrimidin-4-yl)-3-ethyl- | |
| | 1H-pyrrolo[3,2-c]pyridin-6-yl)acetamide | |
| 923 | 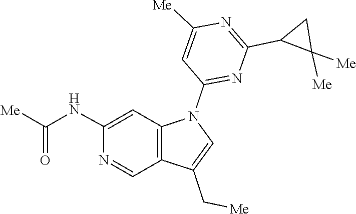 [SMILES: CCc1cn(-c2cc(C)nc(C3CC3(C)C)n2)c2cc(NC(C)=O)ncc12] | LCMS m/z = 364 [M + H]⁺ 1H NMR (400 MHz, CDCl₃) δ 9.10- 9.20 (m, 1H), 8.50-8.60 (m, 2H), 7.55 (s, 1H), 7.07 (s, 1H), 2.81 (dq, 2H, J = 1.0, 7.5 Hz), 2.57 (s, 3H), 2.20-2.30 (m, 4H), 1.79 (t, 1H, J = 5.0 Hz), 1.39 (t, 3H, J = 7.5 Hz), 1.32 (s, 3H), 1.17 (s, 3H), 1.08 (dd, 1H, J = 4.5, 8.0 Hz). |
| | N-(1-(2-(2,2-dimethylcyclopropyl)-6- | |
| | methylpyrimidin-4-yl)-3-ethyl-1H- | |
| | pyrrolo[3,2-c]pyridin-6-yl)acetamide | |
| 924 | 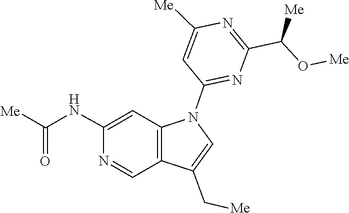 [SMILES: CCc1cn(-c2cc(C)nc([C@@H](C)OC)n2)c2cc(NC(C)=O)ncc12] | LCMS m/z = 354 [M + H]⁺ 1H NMR (400 MHz, CDCl₃) δ 9.10- 9.30 (m, 1H), 8.54 (s, 1H), 8.47 (br s, 1H), 7.59 (s, 1H), 7.21 (s, 1H), 4.63 (q, 1H, J = 6.5 Hz), 3.46 (s, 3H), 2.82 (dq, 2H, J = 1.3, 7.5 Hz), 2.66 (s, 3H), 2.26 (s, 3H), 1.66 (d, 3H, J = 6.8 Hz), 1.39 (t, 3H, J = 7.5 Hz) |
| | (R)-N-(3-ethyl-1-(2-(1-methoxyethyl)-6- | |
| | methylpyrimidin-4-y1)-1H-pyrrolo[3,2- | |
| | c]pyridin-6-yl)acetamide | |
| 925 | 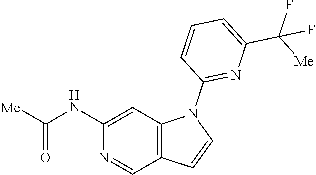 [SMILES: CC(=O)Nc1cc2c(ccn2-c2cccc(C(C)(F)F)n2)cn1] | LCMS m/z = 317 [M + H]⁺ |
| | N-(1-(6-(1,1-difluoroethyl)pyridin-2-yl)- | |
| | 1H-pyrrolo[3,2-c]pyridin-6-yl)acetamide | |
| 926 | 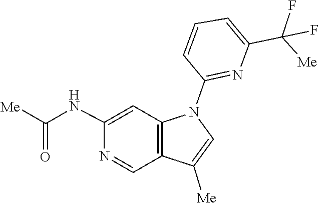 [SMILES: CC(=O)Nc1cc2c(cn1)c(C)cn2-c1cccc(C(C)(F)F)n1] | LCMS m/z = 331 [M + H]⁺ |
| | N-(1-(6-(1,1-difluoroethyl)pyridin-2-yl)- | |
| | 3-methyl-1H-pyrrolo[3,2-c]pyridin-6- | |
| | yl)acetamide | |
| 927 | 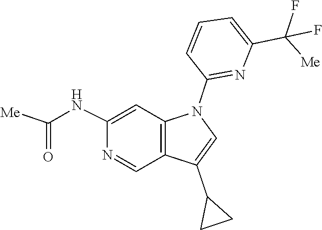 [SMILES: CC(=O)Nc1cc2c(cn1)c(C1CC1)cn2-c1cccc(C(C)(F)F)n1] | LCMS m/z = 357 [M + H]⁺ 1H NMR (MeOD-d₄, 400 MHz) δ 8.88 (s, 1H), 8.45 (s, 1H), 8.20 (t, 1H, J = 7.9 Hz), 8.03 (d, 1H, J = 1.0 Hz), 7.87 (d, 1H, J = 8.0 Hz), 7.73 (d, 1H, J = 7.3 Hz), 2.33 (s, 3H), 2.13 (t, 4H, J = 18.7 Hz), 1.10-1.12 (m, 2H), 0.80-0.90 (m, 2H). |
| | N-(3-cyclopropyl-1-(6-(1,1- | |
| | difluoroethyl)pyridin-2-yl)-1H- | |
| | pyrrolo[3,2-c]pyridin-6-yl)acetamide | |
| 928 | 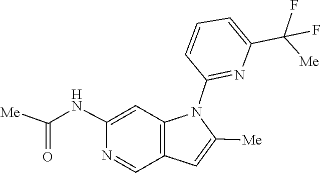 [SMILES: CC(=O)Nc1cc2c(cn1)cc(C)n2-c1cccc(C(C)(F)F)n1] | LCMS m/z = 331 [M + H]⁺ |
| | N-(1-(6-(1,1-difluoroethyl)pyridin-2-yl)- | |
| | 2-methyl-1H-pyrrolo[3,2-c]pyridin-6- | |
| | yl)acetamide | |
| 929 | 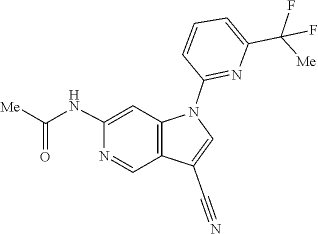 [SMILES: CC(=O)Nc1cc2c(cn1)c(C#N)cn2-c1cccc(C(C)(F)F)n1] | LCMS m/z = 342 [M + H]⁺ |
| | N-(3-cyano-1-(6-(1,1- | |
| | difluoroethyl)pyridin-2-y1)-1H- | |
| | pyrrolo[3,2-c]pyridin-6-yl)acetamide | |
| 930 | 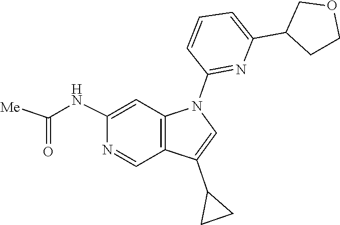 [SMILES: CC(=O)Nc1cc2c(cn1)c(C1CC1)cn2-c1cccc(C2CCOC2)n1] | LCMS m/z = 363 [M + H]⁺ 1H NMR (MeOD-d₄, 400 MHz) δ 9.00 (s, 1H), 8.66 (d, 1H, J = 0.8 Hz), 7.80-7.90 (m, 1H), 7.56 (d, 1H, J = 1.0 Hz), 7.43 (d, 1H, J = 8.0 Hz), 7.20-7.22 (m, 1H), 4.24 (t, 1H, J = 7.9 Hz), 4.10-4.20 (m, 1H), 3.90-4.00 (m, 2H), 3.70 (quin, 1H, J = 7.8 Hz), 2.40-2.50 (m, 2H), 2.20 (s, 3H), 2.00-2.10 (m, 1H), 1.00- 1.02 (m, 2H), 0.70-0.80 (m, 2H). |
| | N-(3-cyclopropyl-1-(6-(tetrahydrofuran-3- | |
| | yl)pyridin-2-yl)-1H-pyrrolo[3,2-c]pyridin- | |
| | 6-yl)acetamide | |
| 931 | 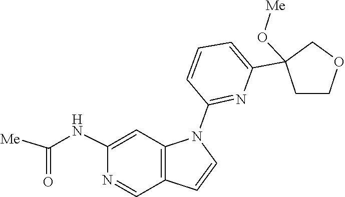 [SMILES: COC1(c2cccc(-n3ccc4cnc(NC(C)=O)cc43)n2)CCOC1] | LCMS m/z = 353 [M + H]⁺ |
| | N-(1-(6-(3-methoxytetrahydrofuran-3- | |
| | yl)pyridin-2-yl)-1H-pyrrolo[3,2-c]pyridin- | |
| | 6-yl)acetamide | |
| 932 | 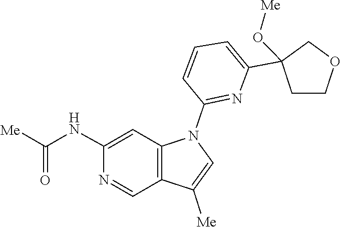 [SMILES: COC1(c2cccc(-n3cc(C)c4cnc(NC(C)=O)cc43)n2)CCOC1] | LCMS m/z = 367 [M + H]⁺ |
| | N-(1-(6-(3-methoxytetrahydrofuran-3- | |
| | yl)pyridin-2-y1)-3-methyl-1H-pyrrolo[3,2- | |
| | c]pyridin-6-yl)acetamide | |
| 933 | 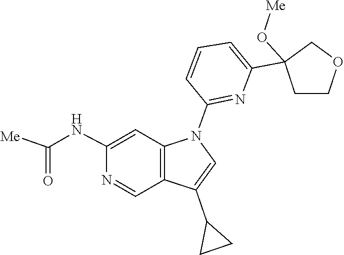 [SMILES: COC1(c2cccc(-n3cc(C4CC4)c4cnc(NC(C)=O)cc43)n2)CCOC1] | LCMS m/z = 393 [M + H]⁺ 1H NMR (CDCl₃, 400 MHz) δ 8.97 (s, 1H), 8.65 (s, 1H), 7.80-7.90 (m, 1H), 7.47 (d, 1H, J = 7.5 Hz), 7.40- 7.42 (m, 2H), 4.20-4.30 (m, 1H), 4.20-4.22 (m, 2H), 4.11 (d, 1H, J = 9.5 Hz), 3.29 (s, 3H), 2.81 (td, 1H, J = 8.4, 13.2 Hz), 2.40-2.50 (m, 1H), 2.26 (s, 3H), 1.90-2.00 (m, 1H), 1.00-1.02 (m, 2H), 0.70-0.80 (m, 2H). |
| | N-(3-cyclopropyl-1-(6-(3- | |
| | methoxytetrahydrofuran-3-y1)pyridin-2- | |
| | yl)-1H-pyrrolo[3,2-c]pyridin-6- | |
| | yl)acetamide | |
| 934 | 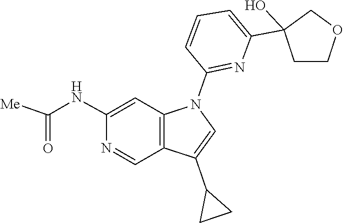 [SMILES: CC(=O)Nc1cc2c(cn1)c(C1CC1)cn2-c1cccc(C2(O)CCOC2)n1] | LCMS m/z = 379 [M + H]⁺ 1H NMR (MeOD-d₄, 400 MHz) δ 8.80-8.90 (m, 1H), 8.30-8.32 (m, 1H), 8.07 (t, 1H, J = 8.0 Hz), 7.95 (d, 1H, J = 0.8 Hz), 7.79 (d, 1H, J = 7.8 Hz), 7.63 (d, 1H, J = 8.3 Hz), 4.20-4.30 (m, 3H), 4.02 (d, 1H, J = 9.0 Hz), 2.70-2.80 (m, 1H), 2.33 (s, 3H), 2.2-2.3 (m, 1H), 2.10-2.12 (m, 1H), 1.10-1.12 (m, 2H), 0.80- 0.90 (m, 2H) |
| | N-(3-cyclopropyl-1-(6-(3- | |
| | hydroxytetrahydrofuran-3-yl)pyridin-2- | |
| | y1)-1H-pyrrolo[3,2-c]pyridin-6- | |
| | yl)acetamide | |
| 935 | 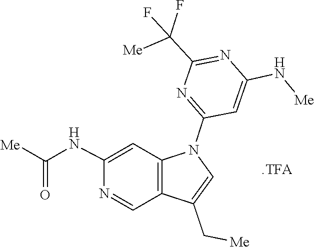 [SMILES: CCc1cn(-c2cc(NC)nc(C(C)(F)F)n2)c2cc(NC(C)=O)ncc12] | LCMS m/z = 375 [M + H]⁺ |
| | N-(1-(2-(1,1-difluoroethyl)-6- | |
| | (methylamino)pyrimidin-4-y1)-3-ethyl- | |
| | 1H-pyrrolo[3,2-c]pyridin-6-yl)acetamide | |
| | trifluoroacetate | |
| 936 | 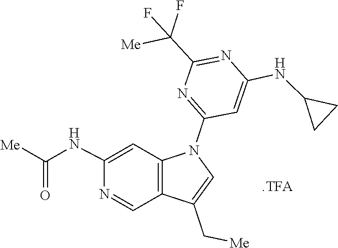 [SMILES: CCc1cn(-c2cc(NC3CC3)nc(C(C)(F)F)n2)c2cc(NC(C)=O)ncc12] | LCMS m/z = 401 [M + H]⁺ |
| | N-(1-(6-(cyclopropylamino)-2-(1,1- | |
| | difluoroethyl)pyrimidin-4-y1)-3-ethyl-1H- | |
| | pyrrolo[3,2-c]pyridin-6-yl)acetamide | |
| | trifluoroacetate | |
| 937 | 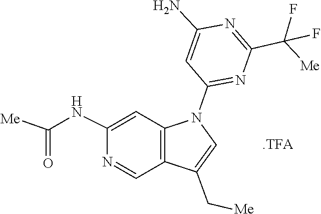 [SMILES: CCc1cn(-c2cc(N)nc(C(C)(F)F)n2)c2cc(NC(C)=O)ncc12] | LCMS m/z = 361 [M + H]⁺ |
| | N-(1-(6-amino-2-(1,1- | |
| | difluoroethyl)pyrimidin-4-yl)-3-ethyl-1H- | |
| | pyrrolo[3,2-c]pyridin-6-yl)acetamide | |
| | trifluoroacetate | |
| 938 | 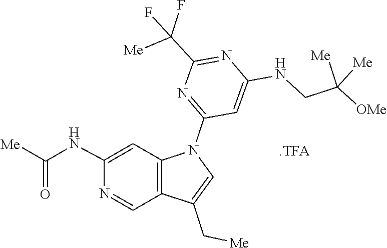 [SMILES: CCc1cn(-c2cc(NCC(C)(C)OC)nc(C(C)(F)F)n2)c2cc(NC(C)=O)ncc12] | LCMS m/z = 447 [M + H]⁺ |
| | N-(1-(2-(1,1-difluoroethyl)-6-((2- | |
| | methoxy-2- | |
| | methylpropyl)amino)pyrimidin-4-y1)-3- | |
| | ethyl-1H-pyrrolo[3,2-c]pyridin-6- | |
| | yl)acetamide trifluoroacetate | |
| 939 | 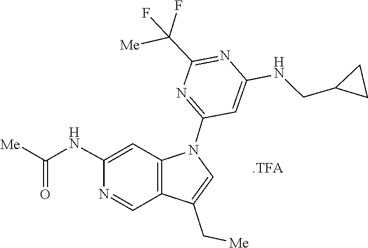 [SMILES: CCc1cn(-c2cc(NCC3CC3)nc(C(C)(F)F)n2)c2cc(NC(C)=O)ncc12] | LCMS m/z = 415 [M + H]⁺ |
| | N-(1-(6-((cyclopropylmethyl)amino)-2- | |
| | (1,1-difluoroethyl)pyrimidin-4-y1)-3- | |
| | ethyl-1H-pyrrolo[3,2-c]pyridin-6- | |
| | yl)acetamide trifluoroacetate | |
| 940 | 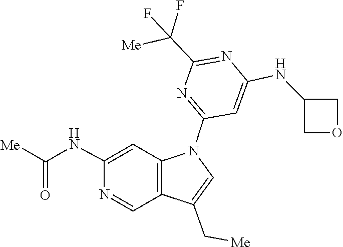 [SMILES: CCc1cn(-c2cc(NC3COC3)nc(C(C)(F)F)n2)c2cc(NC(C)=O)ncc12] | LCMS m/z = 417 [M + H]⁺ |
| | N-(1-(2-(1,1-difluoroethyl)-6-(oxetan-3- | |
| | ylamino)pyrimidin-4-yl)-3-ethyl-1H- | |
| | pyrrolo[3,2-c]pyridin-6-yl)acetamide | |
| 941 | 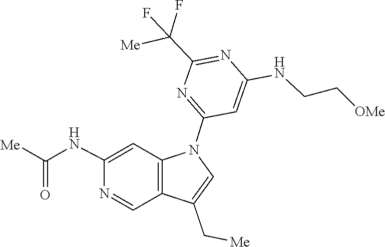 [SMILES: CCc1cn(-c2cc(NCCOC)nc(C(C)(F)F)n2)c2cc(NC(C)=O)ncc12] | LCMS m/z = 419 [M + H]⁺ |
| | N-(1-(2-(1,1-difluoroethyl)-6-((2- | |
| | methoxyethyl)amino)pyrimidin-4-yl)-3- | |
| | ethyl-1H-pyrrolo[3,2-c]pyridin-6- | |
| | yl)acetamide | |
| 942 | 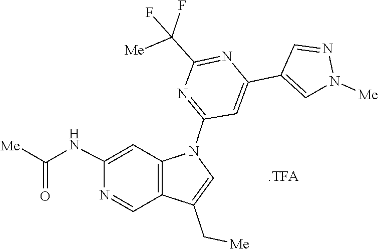 [SMILES: CCc1cn(-c2cc(-c3cnn(C)c3)nc(C(C)(F)F)n2)c2cc(NC(C)=O)ncc12] | LCMS m/z = 426 [M + H]⁺ |
| | N-(1-(2-(1,1-difluoroethyl)-6-(1-methyl- | |
| | 1H-pyrazol-4-yl)pyrimidin-4-y1)-3-ethyl- | |
| | 1H-pyrrolo[3,2-c]pyridin-6-yl)acetamide | |
| | trifluoroacetate | |
| 943 | 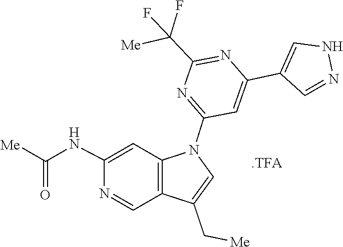 [SMILES: CCc1cn(-c2cc(-c3cn[nH]c3)nc(C(C)(F)F)n2)c2cc(NC(C)=O)ncc12] | LCMS m/z = 412 [M + H]⁺ |
| | N-(1-(2-(1,1-difluoroethyl)-6-(1H- | |
| | pyrazol-4-yl)pyrimidin-4-yl)-3-ethyl-1H- | |
| | pyrrolo[3,2-c]pyridin-6-yl)acetamide | |
| | trifluoroacetate | |
| 944 | 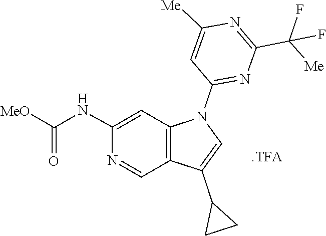 [SMILES: COC(=O)Nc1cc2c(cn1)c(C1CC1)cn2-c1cc(C)nc(C(C)(F)F)n1] | LCMS m/z = 388 [M + H]⁺ |
| | methyl (3-cyclopropyl-1-(2-(1,1- | |
| | difluoroethyl)-6-methylpyrimidin-4-yl)- | |
| | 1H-pyrrolo[3,2-c]pyridin-6-yl)carbamate | |
| | trifluoroacetate | |
| 945 | 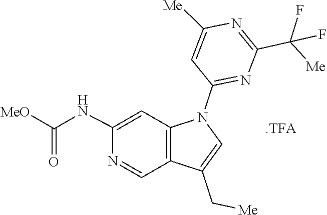 [SMILES: CCc1cn(-c2cc(C)nc(C(C)(F)F)n2)c2cc(NC(=O)OC)ncc12] | LCMS m/z = 376 [M + H]⁺ |
| | methyl (1-(2-(1,1-difluoroethyl)-6- | |
| | methylpyrimidin-4-yl)-3-ethyl-1H- | |
| | pyrrolo[3,2-c]pyridin-6-yl)carbamate | |
| | trifluoroacetate | |
| 946 | 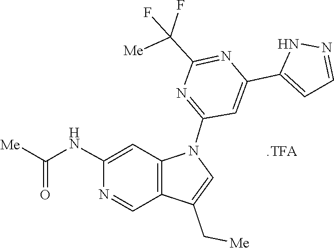 [SMILES: CCc1cn(-c2cc(-c3ccn[nH]3)nc(C(C)(F)F)n2)c2cc(NC(C)=O)ncc12] | LCMS m/z = 412 [M + H]⁺ |
| | N-(1-(2-(1,1-difluoroethyl)-6-(1H- | |
| | pyrazol-5-yl)pyrimidin-4-yl)-3-ethyl-1H- | |
| | pyrrolo[3,2-c]pyridin-6-yl)acetamide | |
| | trifluoroacetate | |
| 947 | 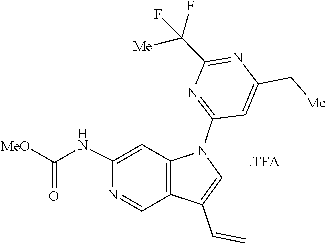 [SMILES: C=Cc1cn(-c2cc(CC)nc(C(C)(F)F)n2)c2cc(NC(=O)OC)ncc12] | LCMS m/z = 388 [M + H]⁺ |
| | methyl (1-(2-(1,1-difluoroethyl)-6- | |
| | ethylpyrimidin-4-y1)-3-vinyl-1H- | |
| | pyrrolo[3,2-c]pyridin-6-yl)carbamate | |
| | trifluoroacetate | |
| 948 | 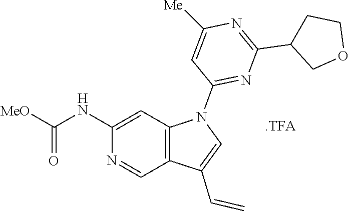 [SMILES: C=Cc1cn(-c2cc(C)nc(C3CCOC3)n2)c2cc(NC(=O)OC)ncc12] | LCMS m/z = 380 [M + H]⁺ |
| | methyl (1-(6-methyl-2-(tetrahydrofuran-3- | |
| | yl)pyrimidin-4-yl)-3-vinyl-1H- | |
| | pyrrolo[3,2-c]pyridin-6-yl)carbamate | |
| 949 | 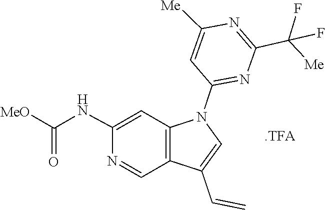 [SMILES: C=Cc1cn(-c2cc(C)nc(C(C)(F)F)n2)c2cc(NC(=O)OC)ncc12] | LCMS m/z = 374 [M + H]⁺ |
| | methyl (1-(2-(1,1-difluoroethyl)-6- | |
| | methylpyrimidin-4-y1)-3-vinyl-1H- | |
| | pyrrolo[3,2-c]pyridin-6-yl)carbamate | |
| | trifluoroacetate | |
| 950 | 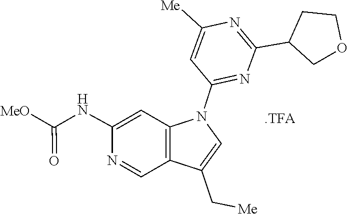 [SMILES: CCc1cn(-c2cc(C)nc(C3CCOC3)n2)c2cc(NC(=O)OC)ncc12] | LCMS m/z = 382 [M + H]⁺ |
| | methyl (3-ethyl-1-(6-methyl-2- | |
| | (tetrahydrofuran-3-yl)pyrimidin-4-yl)-1H- | |
| | pyrrolo[3,2-c]pyridin-6-yl)carbamate | |
| | trifluoroacetate | |
| 951 | 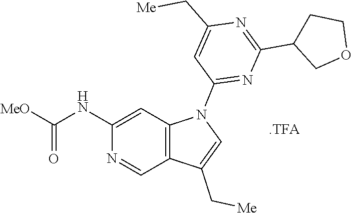 [SMILES: CCc1cc(-n2cc(CC)c3cnc(NC(=O)OC)cc32)nc(C2CCOC2)n1] | LCMS m/z = 396 [M + H]⁺ |
| | methyl (3-ethyl-1-(6-ethyl-2- | |
| | (tetrahydrofuran-3-y1)pyrimidin-4-yl)-1H- | |
| | pyrrolo[3,2-c]pyridin-6-yl)carbamate | |
| | trifluoroacetate | |
| 952 | 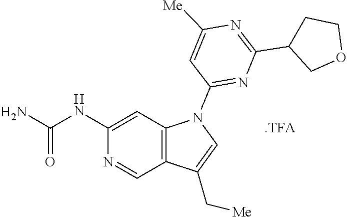 [SMILES: CCc1cn(-c2cc(C)nc(C3CCOC3)n2)c2cc(NC(N)=O)ncc12] | LCMS m/z = 367 [M + H]⁺ |
| | 1-(3-ethyl-1-(6-methyl-2- | |
| | (tetrahydrofuran-3-yl)pyrimidin-4-yl)-1H- | |
| | pyrrolo[3,2-c]pyridin-6-yl)urea | |
| | trifluoroacetate | |
| 953 | 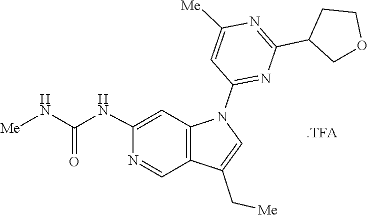 [SMILES: CCc1cn(-c2cc(C)nc(C3CCOC3)n2)c2cc(NC(=O)NC)ncc12] | LCMS m/z = 381 [M + H]⁺ |
| | 1-(3-ethyl-1-(6-methyl-2- | |
| | (tetrahydrofuran-3-y1)pyrimidin-4-y1)-1H- | |
| | pyrrolo[3,2-c]pyridin-6-yl)-3-methylurea | |
| | trifluoroacetate | |
| 954 | 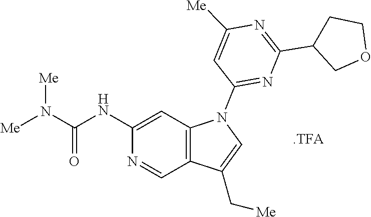 [SMILES: CCc1cn(-c2cc(C)nc(C3CCOC3)n2)c2cc(NC(=O)N(C)C)ncc12] | LCMS m/z = 395 [M + H]⁺ |
| | 3-(3-ethyl-1-(6-methyl-2- | |
| | (tetrahydrofuran-3-yl)pyrimidin-4-yl)-1H- | |
| | pyrrolo[3,2-c]pyridin-6-y1)-1,1- | |
| | dimethylurea trifluoroacetate | |
| 955 | 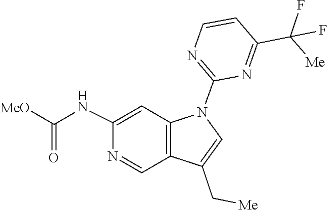 [SMILES: CCc1cn(-c2nccc(C(C)(F)F)n2)c2cc(NC(=O)OC)ncc12] | LCMS m/z = 362 [M + H]⁺ |
| | methyl (1-(4-(1,1-difluoroethyl)pyrimidin- | |
| | 2-y1)-3-ethyl-1H-pyrrolo[3,2-c]pyridin-6- | |
| | yl)carbamate | |
| 956 | 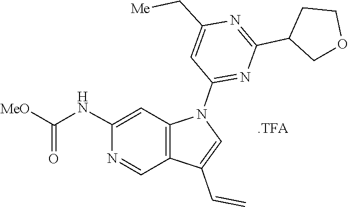 [SMILES: C=Cc1cn(-c2cc(CC)nc(C3CCOC3)n2)c2cc(NC(=O)OC)ncc12] | LCMS m/z = 394 [M + H]⁺ |
| | methyl (1-(6-ethyl-2-(tetrahydrofuran-3- | |
| | y1)pyrimidin-4-yl)-3-vinyl-1H- | |
| | pyrrolo[3,2-c]pyridin-6-y1)carbamate | |
| | trifluoroacetate | |
| 957 | 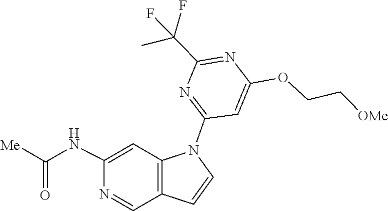 [SMILES: COCCOc1cc(-n2ccc3cnc(NC(C)=O)cc32)nc(C(C)(F)F)n1] | LCMS m/z = 392.2 [M + H]⁺ 1H NMR (600 MHz, DMSO-d₆) (ppm) 10.43 (s, 1H), 9.31 (s, 1H), 8.62 (s, 1H), 8.18 (d, J = 3.6 Hz, 1H), 7.42 (s, 1H), 6.91 (d, J = 3.6 Hz, 1H), 4.62-4.51 (m, 2H), 3.76- 3.64 (m, 2H), 2.52 (s, 3H), 2.20 (s, 3H), 2.10 (s, 3H) |
| | N-(1-(2-(1,1-difluoroethyl)-6-(2- | |
| | methoxyethoxy)pyrimidin-4-y1)-1H- | |
| | pyrrolo[3,2-c]pyridin-6-yl)acetamide | |
| 958 | 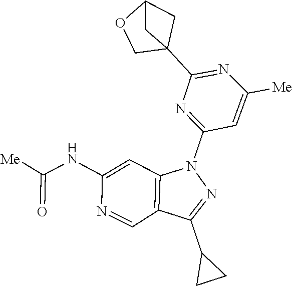 | LCMS m/z = 391.2 [M + H]⁺ |
| | N-(1-(2-(2-oxabicyclo[2.1.1]hexan-4-yl)- | |
| | 6-methylpyrimidin-4-yl)-3-cyclopropyl- | |
| | 1H-pyrazolo[4,3-c]pyridin-6-y1)acetamide | |

### Biological Assays

Compounds of the disclosure were assessed for their ability to inhibit TYK2, JAK1 and JAK2 activity. The inhibitory properties of the compounds of the disclosure described herein can be evidenced by testing in any one of the following protocols.

### JH2 Biochemical Assay

The inhibitory potency of compounds of the disclosure against the kinase activity of recombinantly generated JH2 domain of human Tyk2 was evaluated in a plate-based assay using a TR-FRET assay platform. Briefly, 2 nM of recombinant JH2 domain [10×His-tagged TYK2 JH2 domain (amino acid 575-876)] was combined with 2 nM probe ((S)-6-amino-9-(2-carboxy-4-((1-(3-(8-methyl-5-(methylamino)-8H-imidazo[4,5-d]thiazolo[5,4-b]pyridin-2-yl)phenyl)ethyl)carbamoyl)phenyl)-3-iminio-5-sulfo-3H-xanthene-4-sulfonate), 0.1 nM Tb-labeled anti-His antibody, and compounds of disclosure for 60 minutes. Compounds are tested at either 10 μM or 1 μM top concentration, 10 points of 3-fold dilution. The TR-FRET signal inversely correlates to the amount of probe displaced by compounds and signal was calculated by taking the ratio of fluorescence at 520 nm and 495 nm. The data was normalized and the percent activity versus log concentration of compound was fitted with a 4-parameter logistic model to generate IC50 curves.

pSTAT4 Cell Assay

The inhibitory potency of compounds of the disclosure against the Tyk2 kinase activity on STAT4 was evaluated using an MSD-platform plate-based assay format. NK92 cells natively expressing STAT4 and Tyk2 were serum-starved to reduce background phosphorylation levels, then cells were treated compounds for 1 hr with a 10-point four-fold dilution series starting at 10 μM. Cells were then stimulated with 30 ng/mL IL2 for 15 minutes. Cells were lysed and pSTAT5 levels were quantitated using an MSD plate-based assay with anti-STAT4 antibodies. The data were normalized and the percent activity versus log concentration of compound were fitted with a 4-parameter logistic model to generate to generate IC50 curves.

pSTAT5 Cell Assay

Compounds of the disclosure were assessed for their ability to inhibit the JAK2 kinase activity on STAT5 utilizing an MSD-platform plate-based assay format. TF1 cells natively expressing STAT5 and JAK2 were serum-starved to reduce background phosphorylation levels, then cells were treated with compounds of disclosure for 1 hour with a 10-point four-fold dilution series starting at 10 μM. Cells were then stimulated with 30 ng/mL IL-3 for 15 minutes. Cells were then lysed and pSTAT5 levels were quantitated using an MSD plate-based assay with anti-STAT5 antibodies. The data were normalized and the percent activity versus log concentration of compound was fitted with a 4-parameter logistic model to generate a curve and an IC50 value.

pSTAT3 Cell Assay

The inhibitory potency of compounds of the disclosure against the JAK1 kinase activity on STAT3 was evaluated using an MSD-platform plate-based assay format. TF1 cells natively expressing STAT3 and JAK1 were serum-starved to reduce background phosphorylation levels, then cells were treated with compounds of the disclosure for 1 hour with a 10-point four-fold dilution series starting at 10 μM. Cells were then stimulated with 30 ng/mL interleukin 6 (IL-6) for 15 minutes. Cells were lysed and pSTAT3 levels were quantitated using an MSD plate-based assay with anti-STAT3 antibodies. The data were normalized and the percent activity versus log concentration of compound was fitted with a 4-parameter logistic model to generate IC50 curves.

Table 1 shows the inhibitory activity of selected compounds of this disclosure to assess their ability to inhibit TYK2, JAK1 and JAK2, wherein each compound number corresponds to the compound numbering set forth in Examples 1-957 described herein. The measured IC₅₀ values were scored according to the following hierarchy.

$$“ ++ ++ ” represents : {IC}_{50} \leq 10 nM \\ “ ++ + ” represents : 10 nM < {IC}_{50} \leq 100 nM \\ “ ++ ” represents : 100 nM < {IC}_{50} \leq 1000 nM \\ “ + ” represents : 1000 nM < {IC}_{50}$$

TABLE 1
Experimental Data

| | | TYK2; | JAK2; | JAK1; |
|---|---|---|---|---|
| | TYK2-JH2 | pSTAT4 | pSTAT5 | pSTAT3 |
| Ex. No. | IC₅₀ (nM) | IC₅₀ (nM) | IC₅₀ (nM) | IC₅₀ (nM) |
| 1 | + | − | − | − |
| 2 | ++ | − | − | − |
| 3 | + | − | − | − |
| 4 | ++++ | ++ | + | + |
| 5 | +++ | + | + | + |
| 6 | +++ | ++ | + | + |
| 7 | +++ | ++ | + | + |
| 8 | +++ | ++ | + | + |
| 9 | +++ | ++ | + | + |
| 10 | ++++ | − | − | − |
| 11 | + | − | − | − |
| 12 | +++ | ++ | + | + |
| 13 | ++++ | +++ | + | + |
| 14 | ++++ | +++ | + | + |
| 15 | ++++ | +++ | + | + |
| 16 | ++++ | +++ | + | + |
| 17 | ++++ | +++ | + | + |
| 18 | +++ | ++ | + | + |
| 19 | ++++ | +++ | + | + |
| 20 | ++++ | ++ | + | + |
| 21 | ++++ | ++ | + | + |
| 22 | ++++ | − | − | − |
| 23 | ++++ | +++ | + | + |
| 24 | ++++ | +++ | + | + |
| 25 | ++++ | +++ | + | + |
| 26 | ++++ | ++++ | + | + |
| 27 | ++++ | ++++ | + | + |
| 28 | ++++ | +++ | + | + |
| 29 | ++++ | ++++ | + | + |
| 30 | +++ | ++ | + | + |
| 31 | +++ | + | + | + |
| 32 | ++++ | ++++ | + | + |
| 33 | ++++ | ++++ | + | + |
| 34 | ++++ | ++++ | + | + |
| 35 | ++++ | +++ | + | + |
| 36 | ++++ | ++++ | + | + |
| 37 | ++++ | +++ | + | + |
| 38 | ++++ | +++ | + | + |
| 39 | ++++ | +++ | + | + |
| 40 | +++ | ++ | + | + |
| 41 | ++++ | +++ | + | + |
| 42 | + | − | − | − |
| 43 | + | − | − | − |
| 44 | ++ | − | − | − |
| 45 | +++ | ++ | + | + |
| 46 | + | − | − | − |
| 47 | +++ | − | − | − |
| 48 | +++ | + | + | + |
| 49 | +++ | − | − | − |
| 50 | +++ | − | − | − |
| 51 | ++ | − | − | − |
| 52 | +++ | − | − | − |
| 53 | + | − | − | − |
| 54 | ++ | − | − | − |
| 55 | ++ | − | − | − |
| 56 | +++ | − | − | − |
| 57 | +++ | ++ | + | + |
| 58 | +++ | ++ | + | + |
| 59 | ++++ | +++ | + | + |
| 60 | ++++ | +++ | + | + |
| 61 | ++++ | ++ | + | + |
| 62 | ++++ | ++ | + | + |
| 63 | ++++ | +++ | + | + |
| 64 | ++++ | +++ | + | + |
| 65 | ++++ | ++ | + | + |
| 66 | ++++ | +++ | + | + |
| 67 | ++++ | +++ | + | + |
| 68 | +++ | − | − | − |
| 69 | ++++ | +++ | + | + |
| 70 | +++ | +++ | + | + |
| 71 | ++++ | +++ | + | + |
| 72 | +++ | + | − | − |
| 73 | +++ | ++ | + | + |
| 74 | ++++ | ++ | + | + |
| 75 | ++++ | +++ | + | + |
| 76 | ++++ | +++ | + | + |
| 77 | +++ | +++ | + | + |
| 78 | ++++ | ++ | + | + |
| 79 | ++++ | +++ | + | + |
| 80 | ++++ | ++++ | + | + |
| 81 | ++++ | +++ | + | + |
| 82 | ++ | − | − | − |
| 83 | ++++ | ++ | + | + |
| 84 | +++ | ++ | + | + |
| 85 | ++++ | +++ | + | + |
| 86 | ++++ | ++++ | + | + |
| 87 | +++ | ++ | + | + |
| 88 | +++ | +++ | + | + |
| 89 | ++++ | +++ | + | + |
| 90 | ++ | − | − | − |
| 91 | +++ | − | − | − |
| 92 | +++ | − | − | − |
| 93 | ++++ | +++ | + | + |
| 94 | ++++ | +++ | + | + |
| 95 | ++ | − | − | − |
| 96 | ++++ | +++ | + | + |
| 97 | ++++ | ++++ | + | + |
| 99 | +++ | − | − | − |
| 100 | ++++ | +++ | + | + |
| 101 | ++++ | +++ | + | + |
| 102 | + | − | − | − |
| 103 | + | − | − | − |
| 104 | ++ | − | − | − |
| 105 | ++++ | +++ | + | + |
| 106 | ++++ | +++ | + | + |
| 107 | ++ | − | − | − |
| 108 | + | − | − | − |
| 109 | ++++ | +++ | + | + |
| 110 | ++ | − | − | − |
| 111 | +++ | ++ | + | + |
| 112 | +++ | − | − | − |
| 113 | +++ | − | − | − |
| 114 | +++ | +++ | − | − |
| 115 | ++++ | +++ | + | + |
| 116 | +++ | − | − | − |
| 117 | ++++ | ++++ | + | + |
| 118 | ++++ | +++ | + | + |
| 119 | ++ | − | − | − |
| 120 | +++ | + | + | + |
| 121 | ++++ | +++ | + | + |
| 122 | +++ | ++ | + | + |
| 123 | ++++ | +++ | + | + |
| 124 | ++++ | +++ | + | + |
| 125 | +++ | − | − | − |
| 126 | ++++ | +++ | + | + |
| 127 | ++++ | ++++ | + | + |
| 128 | +++ | ++ | + | + |
| 129 | ++++ | +++ | + | + |
| 130 | ++++ | +++ | + | + |
| 131 | ++++ | +++ | + | + |
| 132 | ++++ | ++ | + | + |
| 133 | ++++ | +++ | + | + |
| 134 | ++ | − | − | − |
| 135 | ++++ | ++ | + | + |
| 136 | ++++ | +++ | + | + |
| 137 | ++++ | +++ | + | + |
| 138 | ++++ | ++ | + | + |
| 139 | +++ | − | − | − |
| 140 | ++++ | +++ | + | + |
| 141 | ++++ | +++ | + | + |
| 142 | +++ | − | − | − |
| 143 | +++ | − | − | − |
| 144 | ++ | − | − | − |
| 145 | +++ | − | − | − |
| 146 | ++ | − | − | − |
| 147 | +++ | − | − | − |
| 148 | ++++ | +++ | + | + |
| 149 | ++++ | ++ | + | + |
| 150 | ++++ | +++ | + | + |
| 151 | ++ | − | − | − |
| 152 | ++++ | ++++ | + | + |
| 153 | ++++ | ++++ | + | + |
| 154 | ++++ | ++++ | + | + |
| 155 | ++++ | +++ | + | + |
| 156 | ++++ | +++ | + | + |
| 157 | +++ | + | + | + |
| 158 | ++++ | +++ | + | + |
| 159 | +++ | ++ | + | + |
| 160 | +++ | − | − | − |
| 161 | ++++ | +++ | + | + |
| 162 | +++ | ++ | + | + |
| 163 | ++++ | ++ | + | + |
| 164 | ++ | − | − | − |
| 165 | + | − | − | − |
| 166 | ++++ | ++++ | + | + |
| 167 | +++ | ++ | + | + |
| 168 | + | − | − | − |
| 169 | +++ | ++ | + | + |
| 170 | +++ | +++ | + | + |
| 171 | + | ++ | + | + |
| 172 | ++++ | +++ | + | + |
| 173 | ++++ | ++ | + | + |
| 174 | ++++ | +++ | + | + |
| 175 | ++++ | +++ | + | + |
| 176 | ++++ | ++++ | + | + |
| 177 | ++++ | +++ | + | + |
| 178 | ++++ | ++++ | + | + |
| 179 | ++++ | +++ | + | + |
| 180 | ++++ | ++++ | + | + |
| 181 | ++++ | +++ | + | + |
| 182 | ++++ | ++++ | + | + |
| 183 | − | − | − | − |
| 184 | ++++ | ++++ | + | + |
| 185 | ++++ | ++++ | + | + |
| 186 | ++++ | +++ | + | + |
| 187 | ++++ | +++ | + | + |
| 188 | ++++ | +++ | + | + |
| 189 | ++++ | +++ | + | + |
| 190 | ++++ | +++ | + | + |
| 191 | ++++ | +++ | + | + |
| 192 | ++++ | +++ | + | + |
| 193 | ++++ | +++ | + | + |
| 194 | ++++ | +++ | + | + |
| 195 | ++++ | +++ | + | + |
| 196 | ++++ | +++ | + | + |
| 197 | ++++ | +++ | + | + |
| 198 | ++++ | +++ | + | + |
| 199 | ++++ | +++ | + | + |
| 200 | ++++ | +++ | + | + |
| 201 | ++++ | +++ | + | + |
| 202 | ++++ | ++ | + | + |
| 203 | ++++ | +++ | + | + |
| 204 | ++++ | +++ | + | + |
| 205 | ++++ | ++++ | + | + |
| 206 | − | − | − | − |
| 207 | +++ | + | + | + |
| 208 | ++ | − | − | − |
| 209 | ++ | − | − | − |
| 210 | ++ | − | − | − |
| 211 | +++ | + | + | + |
| 212 | ++ | − | − | − |
| 213 | ++++ | +++ | + | + |
| 214 | +++ | ++ | + | + |
| 215 | ++++ | +++ | + | + |
| 216 | ++++ | ++++ | + | + |
| 217 | ++++ | ++++ | + | + |
| 218 | ++++ | ++++ | + | + |
| 219 | ++++ | +++ | − | − |
| 220 | ++++ | +++ | − | − |
| 221 | ++++ | ++ | − | − |
| 222 | ++++ | ++ | − | − |
| 223 | − | +++ | + | + |
| 224 | ++++ | ++++ | + | + |
| 225 | +++ | ++ | + | + |
| 226 | +++ | ++ | + | + |
| 227 | +++ | ++ | − | − |
| 228 | ++++ | ++ | − | − |
| 229 | ++ | − | − | − |
| 230 | ++ | − | − | − |
| 231 | ++ | − | − | − |
| 232 | ++++ | +++ | − | − |
| 233 | +++ | + | − | − |
| 234 | ++++ | ++ | − | − |
| 235 | ++++ | ++++ | + | + |
| 236 | ++++ | ++++ | + | + |
| 237 | ++++ | ++++ | + | + |
| 238 | ++++ | ++++ | + | + |
| 239 | ++++ | ++++ | + | + |
| 240 | ++++ | +++ | + | + |
| 241 | ++++ | +++ | + | + |
| 242 | ++++ | ++++ | + | + |
| 243 | ++++ | ++++ | + | + |
| 244 | ++++ | ++++ | + | + |
| 245 | ++++ | ++++ | + | + |
| 246 | ++++ | ++++ | − | − |
| 247 | ++++ | ++++ | − | − |
| 248 | ++++ | ++ | − | − |
| 249 | ++++ | + | − | − |
| 250 | ++++ | ++ | − | − |
| 251 | ++++ | ++ | − | − |
| 252 | + | − | − | − |
| 253 | +++ | − | − | − |
| 254 | +++ | − | − | − |
| 255 | +++ | − | − | − |
| 256 | ++++ | +++ | + | + |
| 257 | ++ | − | − | − |
| 258 | + | − | − | − |
| 259 | + | − | − | − |
| 260 | ++++ | ++ | + | + |
| 261 | + | − | − | − |
| 262 | + | − | − | − |
| 263 | +++ | + | + | + |
| 264 | + | − | − | − |
| 265 | + | − | − | − |
| 266 | ++ | − | − | − |
| 267 | ++ | − | − | − |
| 268 | ++++ | +++ | + | + |
| 269 | ++++ | ++++ | + | + |
| 270 | ++++ | ++++ | − | − |
| 271 | ++++ | ++++ | + | + |
| 272 | ++++ | ++++ | + | + |
| 273 | ++++ | ++++ | + | + |
| 274 | ++++ | ++++ | + | + |
| 275 | ++++ | ++++ | + | + |
| 276 | − | ++++ | + | + |
| 277 | ++++ | ++++ | + | + |
| 278 | ++++ | ++++ | + | + |
| 279 | ++++ | +++ | + | + |
| 280 | ++++ | +++ | + | + |
| 281 | ++++ | ++ | + | + |
| 282 | ++++ | +++ | + | + |
| 283 | ++++ | ++++ | + | + |
| 284 | ++++ | ++++ | + | + |
| 285 | ++++ | ++++ | + | + |
| 286 | − | − | − | − |
| 287 | ++++ | ++++ | − | − |
| 288 | ++++ | ++++ | + | + |
| 289 | − | − | − | − |
| 290 | ++++ | ++++ | + | + |
| 291 | ++++ | ++++ | + | + |
| 292 | ++++ | ++++ | + | + |
| 293 | − | +++ | + | + |
| 294 | ++++ | ++++ | + | + |
| 295 | ++++ | ++++ | − | − |
| 296 | ++++ | ++++ | − | − |
| 297 | ++++ | ++++ | + | + |
| 298 | ++++ | ++++ | − | − |
| 299 | ++++ | ++++ | + | + |
| 300 | ++++ | ++++ | + | + |
| 301 | ++++ | ++++ | + | + |
| 302 | ++++ | ++++ | + | + |
| 303 | ++++ | ++++ | − | − |
| 304 | ++++ | ++++ | + | + |
| 305 | ++++ | ++++ | + | + |
| 306 | ++++ | ++++ | + | + |
| 307 | ++++ | ++++ | − | − |
| 308 | ++++ | ++++ | + | + |
| 309 | ++++ | ++++ | + | + |
| 310 | ++++ | ++++ | + | + |
| 311 | ++++ | ++++ | + | + |
| 312 | ++++ | ++++ | + | + |
| 313 | ++++ | ++++ | + | + |
| 314 | ++++ | ++++ | + | + |
| 315 | ++++ | ++++ | + | + |
| 316 | ++++ | ++++ | + | + |
| 317 | ++++ | ++++ | + | + |
| 318 | ++++ | ++++ | − | − |
| 319 | ++++ | ++++ | − | − |
| 320 | ++++ | ++++ | + | + |
| 321 | ++++ | +++ | − | − |
| 322 | ++++ | +++ | − | − |
| 323 | ++++ | ++++ | − | − |
| 324 | ++++ | ++++ | + | + |
| 325 | ++++ | ++++ | + | + |
| 326 | ++++ | ++++ | − | − |
| 327 | ++++ | ++++ | − | − |
| 328 | ++++ | ++++ | + | + |
| 329 | ++++ | ++++ | + | + |
| 330 | ++++ | ++++ | + | + |
| 331 | − | ++++ | + | + |
| 332 | ++++ | ++++ | + | + |
| 333 | ++++ | ++++ | + | + |
| 334 | ++++ | ++++ | − | − |
| 335 | ++++ | ++++ | − | − |
| 336 | ++++ | ++++ | − | − |
| 337 | ++++ | ++++ | − | − |
| 338 | ++++ | ++++ | − | − |
| 339 | ++++ | ++++ | − | − |
| 340 | ++++ | +++ | − | − |
| 341 | ++++ | ++++ | + | + |
| 342 | ++++ | +++ | − | − |
| 343 | ++++ | ++++ | + | + |
| 344 | ++++ | ++++ | + | + |
| 345 | ++++ | ++++ | + | + |
| 346 | ++++ | ++++ | + | + |
| 347 | ++++ | ++++ | + | + |
| 348 | ++++ | ++++ | − | − |
| 349 | ++++ | ++++ | + | + |
| 350 | − | ++++ | − | − |
| 351 | ++++ | +++ | − | − |
| 352 | ++++ | ++++ | + | + |
| 353 | ++++ | ++++ | − | − |
| 354 | ++++ | ++++ | + | + |
| 355 | ++++ | ++++ | + | + |
| 356 | − | +++ | − | − |
| 357 | ++++ | ++++ | + | + |
| 358 | ++++ | ++++ | − | − |
| 359 | ++++ | ++++ | − | − |
| 360 | ++++ | ++++ | − | − |
| 361 | ++++ | ++++ | − | − |
| 362 | ++++ | ++++ | − | − |
| 363 | +++ | − | − | − |
| 364 | +++ | + | − | − |
| 365 | ++++ | ++++ | + | + |
| 366 | − | ++++ | − | − |
| 367 | ++++ | ++++ | − | − |
| 368 | ++++ | ++++ | − | − |
| 369 | ++++ | ++++ | − | − |
| 370 | ++++ | +++ | − | − |
| 371 | − | ++++ | + | + |
| 372 | − | − | − | − |
| 373 | − | ++++ | − | − |
| 374 | ++++ | ++++ | − | − |
| 375 | − | ++++ | − | − |
| 376 | ++++ | ++++ | − | − |
| 377 | ++++ | ++++ | − | − |
| 378 | ++++ | ++++ | − | − |
| 379 | − | +++ | − | − |
| 380 | ++++ | ++ | − | − |
| 381 | − | ++ | − | − |
| 382 | − | ++++ | − | − |
| 383 | +++ | +++ | − | − |
| 384 | ++++ | +++ | − | − |
| 385 | ++++ | ++++ | + | + |
| 386 | ++ | − | − | − |
| 387 | ++++ | ++++ | + | + |
| 388 | +++ | − | − | − |
| 389 | ++++ | ++++ | − | − |
| 390 | ++++ | ++++ | + | + |
| 391 | ++++ | +++ | − | − |
| 392 | ++++ | ++ | − | − |
| 393 | ++++ | +++ | − | − |
| 394 | ++++ | +++ | − | − |
| 395 | ++++ | +++ | − | − |
| 396 | − | +++ | − | − |
| 397 | − | +++ | − | − |
| 398 | ++++ | ++++ | + | + |
| 399 | ++++ | ++++ | − | − |
| 400 | ++++ | ++++ | − | − |
| 401 | ++++ | +++ | − | − |
| 402 | ++++ | ++++ | − | − |
| 403 | − | − | − | − |
| 404 | ++++ | ++++ | + | + |
| 405 | +++ | − | − | − |
| 406 | ++++ | +++ | − | − |
| 407 | ++++ | ++ | − | − |
| 408 | ++++ | ++++ | + | + |
| 409 | ++++ | +++ | − | − |
| 410 | ++++ | +++ | − | − |
| 411 | ++++ | +++ | − | − |
| 412 | ++++ | +++ | − | − |
| 413 | ++++ | +++ | + | + |
| 414 | ++++ | +++ | + | + |
| 415 | − | +++ | − | − |
| 416 | ++++ | +++ | + | + |
| 417 | ++++ | ++++ | + | + |
| 418 | − | ++++ | + | + |
| 419 | ++++ | +++ | + | + |
| 420 | ++++ | +++ | + | + |
| 421 | ++++ | +++ | + | +++ |
| 422 | − | +++ | − | − |
| 423 | ++++ | +++ | − | − |
| 424 | ++++ | +++ | − | − |
| 425 | ++++ | +++ | − | − |
| 426 | ++++ | +++ | − | − |
| 427 | ++++ | +++ | − | − |
| 428 | ++++ | ++++ | − | − |
| 429 | − | ++++ | − | − |
| 430 | ++++ | ++++ | − | − |
| 431 | +++ | ++ | − | − |
| 432 | ++++ | ++++ | − | − |
| 433 | ++++ | ++++ | − | − |
| 434 | ++++ | ++++ | +++ | ++ |
| 435 | − | ++++ | + | + |
| 436 | − | +++ | − | − |
| 437 | − | − | − | − |
| 438 | − | ++++ | − | − |
| 439 | ++++ | ++++ | + | + |
| 440 | ++++ | +++ | − | − |
| 441 | ++++ | +++ | + | + |
| 442 | ++++ | +++ | − | − |
| 443 | − | ++++ | + | + |
| 444 | − | ++++ | − | − |
| 445 | ++++ | +++ | − | − |
| 446 | ++++ | + | − | − |
| 447 | ++++ | +++ | − | − |
| 448 | +++ | − | − | − |
| 449 | ++ | − | − | − |
| 450 | ++++ | +++ | + | + |
| 451 | ++++ | +++ | + | + |
| 452 | ++ | − | − | − |
| 453 | ++ | − | − | − |
| 454 | ++ | − | − | − |
| 455 | + | − | − | − |
| 456 | +++ | ++ | + | + |
| 457 | ++ | − | − | − |
| 458 | ++ | − | − | − |
| 459 | ++ | − | − | − |
| 460 | ++ | − | − | − |
| 461 | +++ | + | − | − |
| 462 | ++ | − | − | − |
| 463 | ++++ | ++ | + | + |
| 464 | +++ | ++ | + | + |
| 465 | ++ | + | − | − |
| 466 | ++++ | ++++ | − | − |
| 467 | ++++ | +++ | − | − |
| 468 | ++++ | +++ | − | − |
| 469 | ++++ | ++ | − | − |
| 470 | ++ | − | − | − |
| 471 | ++++ | +++ | − | − |
| 472 | ++++ | +++ | − | − |
| 473 | + | − | − | − |
| 474 | ++++ | ++++ | + | + |
| 475 | − | +++ | − | − |
| 476 | ++++ | ++++ | − | − |
| 477 | − | +++ | − | − |
| 478 | − | +++ | − | − |
| 479 | − | ++ | − | − |
| 480 | +++ | ++ | − | − |
| 481 | − | ++ | − | − |
| 482 | ++++ | ++ | − | − |
| 483 | ++++ | +++ | − | − |
| 484 | ++++ | +++ | − | − |
| 485 | ++++ | ++ | − | − |
| 486 | − | +++ | − | − |
| 487 | ++++ | +++ | − | − |
| 488 | ++++ | +++ | + | + |
| 489 | +++ | + | − | − |
| 490 | ++++ | +++ | + | + |
| 491 | ++++ | +++ | + | + |
| 492 | ++++ | +++ | − | − |
| 493 | ++++ | +++ | − | − |
| 494 | ++++ | ++++ | − | − |
| 495 | ++++ | ++++ | − | − |
| 496 | ++++ | ++++ | − | − |
| 497 | ++++ | +++ | − | − |
| 498 | ++++ | ++++ | + | + |
| 499 | ++++ | +++ | − | − |
| 500 | ++++ | ++++ | − | − |
| 501 | ++++ | ++ | − | − |
| 502 | ++++ | ++ | − | − |
| 503 | ++++ | +++ | − | − |
| 504 | ++++ | ++++ | + | + |
| 505 | ++++ | ++++ | − | − |
| 506 | ++++ | ++++ | − | − |
| 507 | ++++ | ++++ | − | − |
| 508 | ++++ | ++++ | − | − |
| 509 | ++++ | ++++ | − | − |
| 510 | ++++ | ++++ | − | − |
| 511 | ++++ | ++++ | − | − |
| 512 | ++++ | ++++ | + | + |
| 513 | ++++ | +++ | − | − |
| 514 | ++++ | +++ | − | − |
| 515 | ++++ | +++ | − | − |
| 516 | ++++ | +++ | − | − |
| 517 | ++++ | ++++ | − | − |
| 518 | ++++ | ++++ | − | − |
| 519 | ++++ | ++++ | − | − |
| 520 | ++++ | ++++ | − | − |
| 521 | ++++ | ++++ | − | − |
| 522 | ++++ | ++++ | + | ++ |
| 523 | ++++ | ++++ | − | − |
| 524 | ++++ | +++ | + | + |
| 525 | ++++ | ++++ | + | + |
| 526 | ++++ | +++ | − | − |
| 527 | ++++ | ++++ | − | − |
| 528 | ++++ | ++++ | − | − |
| 529 | ++++ | ++ | − | − |
| 530 | ++++ | +++ | − | − |
| 531 | ++++ | ++++ | − | − |
| 532 | ++++ | ++++ | − | − |
| 533 | ++++ | ++++ | − | − |
| 534 | ++++ | ++++ | − | − |
| 535 | ++++ | +++ | − | − |
| 536 | ++++ | ++++ | − | − |
| 537 | − | +++ | − | − |
| 538 | ++++ | ++++ | + | + |
| 539 | ++++ | +++ | − | − |
| 540 | ++++ | ++++ | − | − |
| 541 | ++++ | ++++ | − | − |
| 542 | ++++ | ++++ | + | + |
| 543 | ++++ | ++++ | − | − |
| 544 | ++++ | ++++ | + | − |
| 545 | ++++ | ++++ | − | − |
| 546 | +++ | ++++ | − | − |
| 547 | ++++ | ++++ | − | − |
| 548 | ++++ | +++ | − | − |
| 549 | ++++ | ++++ | − | − |
| 550 | +++ | ++ | − | − |
| 551 | ++++ | +++ | − | − |
| 552 | ++++ | +++ | − | − |
| 553 | ++++ | ++++ | + | + |
| 554 | ++++ | +++ | + | + |
| 555 | ++++ | +++ | + | + |
| 556 | ++++ | ++++ | + | + |
| 557 | ++++ | +++ | + | + |
| 558 | +++ | ++ | + | + |
| 559 | ++++ | ++++ | − | − |
| 560 | ++++ | ++++ | + | + |
| 561 | ++++ | ++++ | + | + |
| 562 | ++++ | ++++ | + | + |
| 563 | ++++ | ++++ | + | + |
| 564 | ++++ | ++++ | + | + |
| 565 | ++++ | ++++ | + | + |
| 566 | ++++ | +++ | + | + |
| 567 | ++++ | ++++ | + | + |
| 568 | ++++ | ++++ | + | + |
| 569 | ++++ | ++++ | + | + |
| 570 | ++++ | ++++ | + | + |
| 571 | ++++ | ++++ | + | + |
| 572 | ++++ | ++++ | + | + |
| 573 | ++++ | ++++ | + | + |
| 574 | ++++ | ++++ | + | + |
| 575 | ++++ | +++ | + | + |
| 576 | +++ | − | − | − |
| 577 | ++++ | ++++ | + | + |
| 578 | ++++ | ++++ | + | + |
| 579 | +++ | ++ | + | + |
| 580 | ++++ | ++++ | + | + |
| 581 | +++ | − | − | − |
| 582 | ++++ | ++++ | + | + |
| 583 | ++++ | ++++ | + | + |
| 584 | ++++ | ++++ | + | + |
| 585 | ++++ | ++++ | + | + |
| 586 | ++++ | +++ | + | + |
| 587 | ++++ | +++ | + | + |
| 588 | ++++ | +++ | + | + |
| 589 | ++++ | ++++ | + | + |
| 590 | ++++ | ++++ | + | + |
| 591 | ++++ | +++ | + | + |
| 592 | ++++ | ++++ | + | ++ |
| 593 | +++ | ++++ | + | + |
| 594 | +++ | ++ | − | − |
| 595 | ++++ | +++ | + | + |
| 596 | ++++ | +++ | + | + |
| 597 | ++++ | +++ | + | + |
| 598 | ++++ | +++ | + | + |
| 599 | ++++ | ++ | + | + |
| 600 | +++ | ++ | − | − |
| 601 | +++ | + | + | + |
| 602 | +++ | + | + | + |
| 603 | +++ | − | − | − |
| 604 | + | − | − | − |
| 605 | +++ | ++ | + | + |
| 606 | +++ | + | + | + |
| 607 | +++ | + | + | + |
| 608 | ++ | − | − | − |
| 609 | ++ | − | − | − |
| 610 | ++ | − | − | − |
| 611 | ++ | − | − | − |
| 612 | ++ | − | − | − |
| 613 | ++ | − | − | − |
| 614 | ++ | − | − | − |
| 615 | ++ | − | − | − |
| 616 | ++ | − | − | − |
| 617 | +++ | − | − | − |
| 618 | +++ | − | − | − |
| 619 | +++ | ++ | + | + |
| 620 | ++ | + | + | + |
| 621 | + | − | − | − |
| 622 | ++ | − | − | − |
| 623 | ++ | − | − | − |
| 624 | ++ | − | − | − |
| 625 | ++ | − | − | − |
| 626 | ++ | − | − | − |
| 627 | ++ | − | − | − |
| 628 | ++ | − | − | − |
| 629 | ++ | − | − | − |
| 630 | +++ | − | − | − |
| 631 | +++ | − | − | − |
| 632 | +++ | ++ | + | + |
| 633 | ++ | − | − | − |
| 634 | +++ | − | − | − |
| 635 | ++ | − | − | − |
| 636 | ++ | − | − | − |
| 637 | + | − | − | − |
| 638 | ++ | − | − | − |
| 639 | ++ | − | − | − |
| 640 | ++ | − | − | − |
| 641 | +++ | ++ | + | + |
| 642 | +++ | − | − | − |
| 643 | ++ | − | − | − |
| 644 | +++ | + | + | + |
| 645 | ++ | − | − | − |
| 646 | ++ | − | − | − |
| 647 | ++ | − | − | − |
| 648 | ++ | − | − | − |
| 649 | ++ | − | − | − |
| 650 | +++ | − | − | − |
| 651 | ++ | − | − | − |
| 652 | ++ | − | − | − |
| 653 | ++ | − | − | − |
| 654 | +++ | − | − | − |
| 655 | + | − | − | − |
| 656 | + | − | − | − |
| 657 | + | − | − | − |
| 658 | ++ | − | − | − |
| 659 | ++ | − | − | − |
| 660 | ++ | − | − | − |
| 661 | ++ | − | − | − |
| 662 | +++ | − | − | − |
| 663 | + | − | − | − |
| 664 | + | − | − | − |
| 665 | + | − | − | − |
| 666 | +++ | − | − | − |
| 667 | +++ | ++ | + | + |
| 668 | + | − | − | − |
| 669 | ++ | − | − | − |
| 670 | + | − | − | − |
| 671 | ++ | − | − | − |
| 672 | + | − | − | − |
| 673 | +++ | ++ | + | + |
| 674 | + | − | − | − |
| 675 | +++ | − | − | − |
| 676 | +++ | + | − | − |
| 677 | ++ | − | − | − |
| 678 | + | − | − | − |
| 679 | ++ | − | − | − |
| 680 | ++ | − | − | − |
| 681 | +++ | − | − | − |
| 682 | ++ | − | − | − |
| 683 | +++ | − | − | − |
| 684 | ++ | − | − | − |
| 685 | ++ | − | − | − |
| 686 | + | − | − | − |
| 687 | ++ | − | − | − |
| 688 | + | − | − | − |
| 689 | ++ | − | − | − |
| 690 | +++ | − | − | − |
| 691 | ++ | − | − | − |
| 692 | + | − | − | − |
| 693 | ++ | − | − | − |
| 694 | ++ | − | − | − |
| 695 | + | − | − | − |
| 696 | +++ | ++ | + | + |
| 697 | ++ | − | − | − |
| 698 | +++ | + | + | + |
| 699 | ++++ | ++ | + | + |
| 700 | +++ | + | + | + |
| 701 | ++ | − | − | − |
| 702 | +++ | ++ | + | + |
| 703 | +++ | − | − | − |
| 704 | +++ | − | − | − |
| 705 | ++ | − | − | − |
| 706 | ++ | − | − | − |
| 707 | − | + | + | + |
| 708 | +++ | + | − | − |
| 709 | +++ | + | − | − |
| 710 | +++ | + | − | − |
| 711 | ++++ | ++ | − | − |
| 712 | +++ | − | − | − |
| 713 | ++++ | ++ | − | − |
| 714 | +++ | + | − | − |
| 715 | ++++ | ++ | − | − |
| 716 | +++ | + | − | − |
| 717 | ++++ | ++ | − | − |
| 718 | ++ | + | − | − |
| 719 | +++ | + | − | − |
| 720 | ++ | + | − | − |
| 721 | +++ | + | − | − |
| 722 | +++ | + | − | − |
| 723 | ++ | ++ | − | − |
| 724 | ++ | + | − | − |
| 725 | ++ | ++ | − | − |
| 726 | +++ | + | − | − |
| 727 | ++++ | ++ | − | − |
| 728 | +++ | + | − | − |
| 729 | ++ | + | − | − |
| 730 | ++++ | +++ | − | − |
| 731 | ++++ | ++ | − | − |
| 732 | +++ | ++ | − | − |
| 733 | ++++ | ++ | − | − |
| 734 | +++ | ++ | − | − |
| 735 | +++ | + | − | − |
| 736 | +++ | + | − | − |
| 737 | ++++ | ++ | − | − |
| 738 | +++ | + | − | − |
| 739 | ++++ | ++ | − | − |
| 740 | +++ | + | − | − |
| 741 | +++ | ++ | − | − |
| 742 | +++ | + | − | − |
| 743 | − | ++ | − | − |
| 744 | ++++ | +++ | − | − |
| 745 | − | ++ | − | − |
| 746 | − | + | − | − |
| 747 | +++ | + | − | − |
| 748 | ++++ | ++ | − | − |
| 749 | ++++ | ++ | − | − |
| 750 | − | +++ | − | − |
| 751 | +++ | + | − | − |
| 752 | − | − | − | − |
| 753 | ++++ | ++ | − | − |
| 754 | +++ | ++ | − | − |
| 755 | ++++ | +++ | − | − |
| 756 | − | ++ | − | − |
| 757 | +++ | − | − | − |
| 758 | − | − | − | − |
| 759 | +++ | + | − | − |
| 760 | +++ | ++ | − | − |
| 761 | +++ | + | − | − |
| 762 | − | − | − | − |
| 763 | − | ++ | − | − |
| 764 | +++ | − | − | − |
| 765 | +++ | − | − | − |
| 766 | ++++ | +++ | − | − |
| 767 | +++ | ++ | − | − |
| 768 | − | + | − | − |
| 769 | +++ | − | − | − |
| 770 | ++++ | ++ | − | − |
| 771 | +++ | + | − | − |
| 772 | − | + | − | − |
| 773 | ++++ | ++ | − | − |
| 774 | ++++ | +++ | + | + |
| 775 | ++++ | +++ | + | + |
| 776 | ++++ | +++ | + | + |
| 777 | +++ | ++ | + | + |
| 778 | ++++ | +++ | + | + |
| 779 | ++++ | +++ | + | + |
| 780 | ++++ | ++++ | + | + |
| 781 | ++++ | +++ | + | + |
| 782 | ++++ | +++ | + | + |
| 783 | ++++ | +++ | + | + |
| 784 | ++++ | +++ | + | + |
| 785 | + | − | − | − |
| 786 | ++++ | ++ | + | + |
| 787 | ++++ | ++++ | + | + |
| 788 | +++ | ++ | + | + |
| 789 | ++ | − | − | − |
| 790 | +++ | + | + | + |
| 791 | +++ | ++ | + | + |
| 792 | ++++ | +++ | + | + |
| 793 | ++++ | +++ | + | + |
| 794 | +++ | ++ | − | − |
| 795 | +++ | ++ | − | − |
| 796 | +++ | − | − | − |
| 797 | +++ | − | − | − |
| 798 | +++ | ++ | − | − |
| 799 | ++ | − | − | − |
| 800 | +++ | +++ | − | − |
| 801 | +++ | ++ | − | − |
| 802 | ++++ | +++ | − | − |
| 803 | ++++ | ++ | + | + |
| 804 | +++ | +++ | + | + |
| 805 | ++ | − | − | − |
| 806 | ++++ | +++ | + | + |
| 807 | ++++ | +++ | + | + |
| 808 | ++++ | +++ | + | + |
| 809 | ++++ | +++ | + | + |
| 810 | ++++ | +++ | + | + |
| 811 | ++++ | +++ | + | + |
| 812 | ++++ | ++ | + | + |
| 813 | +++ | ++ | + | + |
| 814 | ++++ | ++ | + | + |
| 815 | ++++ | ++ | + | + |
| 816 | ++++ | +++ | + | + |
| 817 | ++++ | ++ | + | + |
| 818 | ++++ | +++ | + | + |
| 819 | ++++ | ++++ | + | + |
| 820 | ++++ | ++++ | + | + |
| 821 | ++++ | +++ | + | + |
| 822 | ++++ | +++ | + | + |
| 823 | +++ | ++ | + | + |
| 824 | ++++ | +++ | + | + |
| 825 | ++ | − | − | − |
| 826 | ++++ | +++ | − | − |
| 827 | ++++ | +++ | − | − |
| 828 | ++++ | +++ | − | − |
| 829 | ++ | − | − | − |
| 830 | ++ | − | − | − |
| 831 | ++ | − | − | − |
| 832 | +++ | − | − | − |
| 833 | +++ | − | − | − |
| 834 | ++ | − | − | − |
| 835 | +++ | − | − | − |
| 836 | ++++ | +++ | + | + |
| 837 | +++ | ++ | + | + |
| 838 | +++ | ++ | + | + |
| 839 | +++ | − | − | − |
| 840 | +++ | ++ | + | + |
| 841 | ++++ | ++ | + | + |
| 842 | ++++ | +++ | + | + |
| 843 | +++ | − | − | − |
| 844 | ++++ | +++ | + | + |
| 845 | ++++ | +++ | + | + |
| 846 | ++++ | ++++ | + | + |
| 847 | ++++ | ++++ | + | + |
| 848 | ++++ | +++ | + | + |
| 849 | ++++ | +++ | + | + |
| 850 | ++++ | ++++ | + | + |
| 851 | ++++ | +++ | + | + |
| 852 | ++++ | +++ | + | + |
| 853 | ++++ | ++++ | + | + |
| 854 | ++++ | +++ | − | − |
| 855 | ++++ | ++ | − | − |
| 856 | ++++ | ++ | − | − |
| 857 | ++++ | ++ | − | − |
| 858 | − | − | − | − |
| 859 | +++ | + | + | + |
| 860 | +++ | + | + | + |
| 861 | +++ | ++ | + | + |
| 862 | ++++ | ++ | + | + |
| 863 | +++ | − | − | − |
| 864 | ++++ | +++ | + | + |
| 865 | ++++ | +++ | + | + |
| 866 | ++++ | +++ | + | + |
| 867 | ++++ | +++ | + | + |
| 868 | ++++ | − | − | − |
| 869 | +++ | ++ | + | + |
| 870 | ++++ | ++ | + | + |
| 871 | ++++ | +++ | + | + |
| 872 | ++++ | ++ | + | + |
| 873 | ++++ | +++ | + | + |
| 874 | +++ | − | − | − |
| 875 | ++++ | +++ | + | + |
| 876 | ++ | + | − | − |
| 877 | ++++ | ++ | + | + |
| 878 | ++++ | ++ | + | + |
| 879 | ++ | − | − | − |
| 880 | ++ | − | − | − |
| 881 | ++ | − | − | − |
| 882 | ++ | − | − | − |
| 883 | ++++ | ++ | − | − |
| 884 | ++++ | +++ | − | − |
| 885 | − | + | − | − |
| 886 | ++++ | ++ | − | − |
| 887 | ++++ | ++ | − | − |
| 888 | ++++ | ++ | − | − |
| 889 | ++++ | ++++ | + | + |
| 890 | ++ | − | − | − |
| 891 | +++ | − | − | − |
| 892 | ++++ | ++ | + | + |
| 893 | +++ | − | − | − |
| 894 | ++++ | +++ | + | + |
| 895 | ++++ | +++ | − | − |
| 896 | +++ | +++ | + | + |
| 897 | +++ | +++ | + | + |
| 898 | +++ | − | − | − |
| 899 | ++++ | ++++ | + | + |
| 900 | +++ | ++ | + | + |
| 901 | ++++ | ++ | + | + |
| 902 | ++ | − | − | − |
| 903 | ++++ | +++ | + | + |
| 904 | +++ | − | − | − |
| 905 | ++++ | ++ | + | + |
| 906 | +++ | + | + | + |
| 907 | ++++ | +++ | + | + |
| 908 | ++++ | ++++ | + | + |
| 909 | +++ | + | + | + |
| 910 | +++ | + | + | + |
| 911 | ++++ | +++ | + | + |
| 912 | − | +++ | − | − |
| 913 | ++++ | +++ | + | + |
| 914 | − | ++ | − | − |
| 915 | ++++ | +++ | − | − |
| 916 | ++++ | +++ | − | − |
| 917 | ++++ | +++ | − | − |
| 918 | ++++ | +++ | − | − |
| 919 | ++++ | +++ | − | − |
| 920 | ++++ | +++ | − | − |
| 921 | ++++ | +++ | − | − |
| 922 | − | ++ | − | − |
| 923 | ++++ | +++ | − | − |
| 924 | ++++ | +++ | − | − |
| 925 | +++ | ++ | + | + |
| 926 | ++++ | ++++ | + | + |
| 927 | +++ | ++++ | + | + |
| 928 | ++ | − | − | − |
| 929 | +++ | − | − | − |
| 930 | ++++ | +++ | + | + |
| 931 | +++ | + | + | + |
| 932 | ++++ | +++ | + | + |
| 933 | ++++ | +++ | + | + |
| 934 | ++++ | +++ | + | + |
| 935 | ++++ | +++ | − | − |
| 936 | ++++ | +++ | − | − |
| 937 | ++++ | ++++ | − | − |
| 938 | ++++ | +++ | − | − |
| 939 | ++++ | ++++ | − | − |
| 940 | ++++ | +++ | − | − |
| 941 | ++++ | ++++ | − | − |
| 942 | ++++ | ++++ | − | − |
| 943 | ++++ | +++ | − | − |
| 944 | ++++ | ++++ | + | + |
| 945 | − | − | − | − |
| 946 | ++++ | − | − | − |
| 947 | ++++ | +++ | − | − |
| 948 | +++ | ++ | − | − |
| 949 | ++++ | +++ | − | − |
| 950 | ++++ | +++ | − | − |
| 951 | ++++ | +++ | − | − |
| 952 | ++++ | ++++ | − | − |
| 953 | ++++ | +++ | − | − |
| 954 | +++ | ++ | − | − |
| 955 | − | ++ | − | − |
| 956 | +++ | ++ | − | − |
| 957 | +++ | ++ | + | + |

### EQUIVALENTS

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims are introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the disclosure, or aspects of the disclosure, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the disclosure, to the tenth of the unit of the lower limit of the range, unless the context clearly dictates otherwise.

Those skilled in the art will recognize or be able to ascertain using no more than routine experimentation, many equivalents to the specific embodiments of the disclosure described and claimed herein. Such equivalents are intended to be encompassed by the following claims.

What is claimed is:

1. A compound of Formula (I): 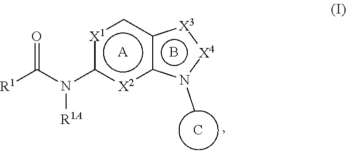
or a pharmaceutically acceptable salt thereof, wherein:
ring A is an aromatic or heteoaromatic ring fused with ring B that is a 5-membered heteroaromatic ring;
X¹ is N or CH;
X² is N or CR²;
X³ is N or CR³;
X⁴ is N or CR⁴;
ring C is phenyl, 5 or 6 membered monocyclic heterocyclyl, or 5 to 6 membered heteroaryl, each of which is optionally substituted by one or more R^{C};
each R^{C} is independently halo, —CN, —NR^{N1}R^{N2}, —NR^{N3}—C(O)—R⁷, —NR^{N4}—SO₂—R⁷, —C(O)—R⁷, —SO₂—R⁷, —OR^{O1}, C₁₋₆ alkyl, alkenyl, 3 to 7 membered monocyclic carbocyclyl, phenyl, 5 to 12 membered monocyclic or bicyclic heteroaryl, or 4 to 9 membered monocyclic or bicyclic heterocyclyl, wherein the C₁₋₆ alkyl, 3 to 7 membered monocyclic carbocyclyl, phenyl, 5 to 12 membered monocyclic or bicyclic heteroaryl, and 4 to 9 membered monocyclic or bicyclic heterocyclyl represented by R^{C} are each optionally substituted with one or more R^{C1}, or two R^{C} taken together with intervening atoms form a 3 to 7 membered monocyclic carbocyclyl optionally substituted with one or more halo;
each R^{C1} is independently halo, oxo, —CN, —OR^{O1}, —NR^{N1}R^{N2}, —C(O)—R⁷, —C(O)—OR^{O3}, —SO₂—R⁷, C₁₋₆ alkyl, C₃₋₆cycloalkyl, phenyl, 5 to 12 membered monocyclic or bicyclic heteroaryl, or 4 to 7 membered monocyclic heterocyclyl, wherein the C₁₋₆ alkyl, phenyl, 5 to 6 membered heteroaryl and 4 to 7 membered monocyclic heterocyclyl represented by R^{C1} are each optionally substituted by one or more substituents independently selected from halo, oxo, —CN, —OR^{O1}, —NR^{N1}R^{N2}, C₁₋₆ alkyl, C₁₋₄ haloalkyl, phenyl, 5 to 6 membered heteroaryl, 3 to 7 membered monocyclic carbocyclyl and 4 to 8 membered monocyclic heterocyclyl;
R¹ is H, C₁₋₆ alkyl, —OR^{1A}, —NR^{N1}R^{N2}, C₃₋₆ cycloalkyl, phenyl, 5 to 6 membered heteroaryl, 3 to 7 membered monocyclic carbocyclyl or 4 to 7 membered monocyclic heterocyclyl, wherein the C₁₋₆ alkyl, phenyl, C₃₋₆ cycloalkyl, 5 to 6 membered heteroaryl, 3 to 7 membered monocyclic carbocyclyl and 4 to 7 membered monocyclic heterocyclyl represented by R¹ are each optionally substituted by one or more R⁸;
R^{1A} is H or C₁₋₃alkyl;
or R¹ and R^{1A} together with the atom from which they are attached form a 5 or 6 membered monocyclic heterocycle;
R² is H or halo;
R³ is H, —NR^{N1}R^{N2}, —CN, halo, —C(O)—R⁷, —C(O)—OR^{O3}, —SO₂—R⁷, —OR^{O4}, C₁₋₆ alkyl, alkenyl, phenyl, 5 to 6 membered heteroaryl, 3 to 7 membered monocyclic carbocyclyl, or 4 to 9 membered monocyclic or bicyclic heterocyclyl, wherein the C₁₋₆ alkyl, phenyl, 5 to 6 membered heteroaryl, 3 to 7 membered monocyclic carbocyclyl and 4 to 9 membered monocyclic or bicyclic heterocyclyl represented by R³ are each optionally substituted by one or more R⁹;
R⁴ is H or halo;
each R⁷ is independently C₁₋₆ alkyl, phenyl, 5 to 6 membered heteroaryl, 3 to 7 membered monocyclic carbocyclyl or 4 to 7 membered monocyclic heterocyclyl; wherein the C₁₋₆ alkyl, phenyl, 5 to 6 membered heteroaryl, 3 to 7 membered monocyclic carbocyclyl and 4 to 7 membered monocyclic heterocyclyl represented by R⁷ are each optionally substituted by one or more substituents independently selected from halo, oxo, —CN, —OR^{O1}, —NR^{1a}R^{1b}, C₁₋₆ alkyl, C₁₋₄ haloalkyl, 3 to 7 membered monocyclic carbocyclyl and 4 to 7 membered monocyclic heterocyclyl;
each R⁸ is independently halo, oxo, —CN, —OR^{O1}, C₁₋₆ alkyl, C₁₋₄ haloalkyl, phenyl, 5 to 6 membered heteroaryl, 3 to 7 membered monocyclic carbocyclyl or 4 to 7 membered monocyclic heterocyclyl;
each R⁹ is independently halo, oxo, —OR^{O1}, —NR^{N1}R^{N2}, —CN, —C(O)—OR^{O3}, —SO₂—R¹⁰, C₁₋₆ alkyl, phenyl, 5 to 6 membered heteroaryl, 3 to 7 membered monocyclic carbocyclyl or 4 to 10 membered monocyclic or bicyclic heterocyclyl, wherein the C₁₋₆ alkyl, phenyl, 5 to 6 membered heteroaryl, 3 to 7 membered monocyclic carbocyclyl and 4 to 10 membered monocyclic or bicyclic heterocyclyl represented by R⁹ are each optionally substituted by one or more substituents independently selected from halo, oxo, —CN, —OR^{O1}, —N^{N1}R^{N2}, C₁₋₆ alkyl, C₁₋₄ haloalkyl, phenyl, 5 to 6 membered heteroaryl, 3 to 7 membered monocyclic carbocyclyl and 4 to 7 membered monocyclic heterocyclyl;
each R^{O1} is independently H, C₁₋₆ alkyl, phenyl, 5 to 6 membered heteroaryl, 3 to 7 membered monocyclic or bicyclic carbocyclyl, or 4 to 7 membered monocyclic or bicyclic heterocyclyl, wherein the C₁₋₆ alkyl, phenyl, 5 to 6 membered heteroaryl, 3 to 7 membered monocyclic or bicyclic carbocyclyl, and 4 to 7 membered monocyclic or bicyclic heterocyclyl represented by R^{O1} are each optionally substituted by one or more R^{O2};
each R^{O2} is independently halo, OH, —CN, C₁₋₄ alkoxy, C₁₋₄ alkyl, phenyl, 5 to 6 membered heteroaryl, 3 to 7 membered monocyclic carbocylyl or 4 to 7 membered monocyclic or bicyclic heterocyclyl, wherein the C₁₋₄ alkyl, phenyl, 5 to 6 membered heteroaryl, 3 to 7 membered monocyclic carbocylyl and 4 to 7 membered monocyclic or bicyclic heterocyclyl are each optionally substituted with one or more halo, C₁₋₆ alkyl or —O—C₁₋₆alkyl;
each R^{O3} is independently H, C₁₋₆ alkyl, C₁₋₄ haloalkyl, phenyl, 5 to 6 membered heteroaryl, 3 to 7 membered monocyclic carbocyclyl or 4 to 7 membered monocyclic heterocyclyl, wherein the C₁₋₆ alkyl, phenyl, 5 to 6 membered heteroaryl, 3 to 7 membered monocyclic carbocyclyl and 4 to 7 membered monocyclic heterocyclyl represented by R^{O3} are each optionally substituted by one or more R^{O2};
R^{O4} is H, C₁₋₆ alkyl, phenyl, 5 to 6 membered heteroaryl, 3 to 7 membered monocyclic carbocyclyl or 4 to 7 membered monocyclic heterocyclyl, wherein the C₁₋₆ alkyl, phenyl, 5 to 6 membered heteroaryl, 3 to 7 membered monocyclic carbocyclyl and 4 to 7 membered monocyclic heterocyclyl represented by R^{O4} are each optionally substituted by one or more substituents independently selected from halo, oxo, —CN, —OR^{O1}, —NR^{N1}R^{N2}, C₁₋₆ alkyl, C₁₋₄ haloalkyl, phenyl, 5 to 6 membered heteroaryl, 3 to 7 membered monocyclic carbocyclyl and 4 to 7 membered monocyclic heterocyclyl;
R^{N1} and R^{N2} are each independently H, C₁₋₆ alkyl, 4 to 7 membered monocyclic heterocyclyl, 5 or 6 membered heteroaryl, or C₃₋₆ cycloalkyl, wherein the C₁₋₆ alkyl represented by R^{N1} and R^{N2} are each optionally substituted with C₁₋₄alkoxy or phenyl, and wherein the C₃₋₆ cycloalkyl, 4 to 7 membered monocyclic heterocyclyl, 5 or 6 membered heteroaryl represented by R^{N1} and R^{N2} are each optionally substituted with C₁₋₄alkyl;
each R^{N3} is independently H or C₁₋₆ alkyl; and
each R^{N4} is independently H or C₁₋₆ alkyl.

2. The compound of claim 1, wherein:
ring A is an aromatic or heteoaromatic ring fused with ring B that is a 5-membered heteroaromatic ring;
X¹ is N or CH;
X² is N or CR²;
X³ is N or CR³;
X⁴ is N or CR⁴;
ring C is phenyl or 5 to 6 membered heteroaryl, each of which is optionally substituted by one or more R^{C};
each R^{C} is independently halo, —CN, —NR^{N1}R^{N2}, —NR^{N3}—C(O)—R⁷, —NR^{N4}—SO₂—R⁷, —C(O)—R⁷, —SO₂—R⁷, —OR^{O1}, C₁₋₆ alkyl, 3 to 7 membered monocyclic carbocyclyl or 4 to 9 membered monocyclic or bicyclic heterocyclyl, wherein the C₁₋₆ alkyl, 3 to 7 membered monocyclic carbocyclyl and 4 to 9 membered monocyclic or bicyclic heterocyclyl represented by R^{C} are each optionally substituted with one or more R^{C1}, or two R^{C} taken together with intervening atoms form a 3 to 7 membered monocyclic carbocyclyl optionally substituted with one or more halo;
each R^{C1} is independently halo, oxo, —CN, —OR^{O1}, —NR^{N1}R^{N2}, —C(O)—R⁷, —C(O)—OR^{O3}, —SO₂—R⁷, C₁₋₆ alkyl, phenyl, 5 to 6 membered heteroaryl, or 4 to 7 membered monocyclic heterocyclyl, wherein the C₁₋₆ alkyl, phenyl, 5 to 6 membered heteroaryl and 4 to 7 membered monocyclic heterocyclyl represented by R^{C1} are each optionally substituted by one or more substituents independently selected from halo, oxo, —CN, —OR^{O1}, —NR^{N1}R^{N2}, C₁₋₆ alkyl, C₁₋₄ haloalkyl, phenyl, 5 to 6 membered heteroaryl, 3 to 7 membered monocyclic carbocyclyl and 4 to 8 membered monocyclic heterocyclyl;
R¹ is C₁₋₆ alkyl, phenyl, 5 to 6 membered heteroaryl, 3 to 7 membered monocyclic carbocyclyl or 4 to 7 membered monocyclic heterocyclyl, wherein the C₁₋₆ alkyl, phenyl, 5 to 6 membered heteroaryl, 3 to 7 membered monocyclic carbocyclyl and 4 to 7 membered monocyclic heterocyclyl represented by R¹ are each optionally substituted by one or more R⁸;
R^{1A} is H or C₁₋₃alkyl;
or R¹ and R^{1A} together with the atom from which they are attached form a 5 or 6 membered monocyclic heterocycle;
R² is H or halo;
R³ is H, —NR^{N1}R^{N2}, —CN, halo, —C(O)—R⁷, —C(O)—OR^{O3}, —SO₂—R⁷, —OR^{O4}, C₁₋₆ alkyl, phenyl, 5 to 6 membered heteroaryl, 3 to 7 membered monocyclic carbocyclyl, or 4 to 9 membered monocyclic or bicyclic heterocyclyl, wherein the C₁₋₆ alkyl, phenyl, 5 to 6 membered heteroaryl, 3 to 7 membered monocyclic carbocyclyl and 4 to 9 membered monocyclic or bicyclic heterocyclyl represented by R³ are each optionally substituted by one or more R⁹;
R⁴ is H or halo;
each R⁷ is independently C₁₋₆ alkyl, phenyl, 5 to 6 membered heteroaryl, 3 to 7 membered monocyclic carbocyclyl or 4 to 7 membered monocyclic heterocyclyl; wherein the C₁₋₆ alkyl, phenyl, 5 to 6 membered heteroaryl, 3 to 7 membered monocyclic carbocyclyl and 4 to 7 membered monocyclic heterocyclyl represented by R⁷ are each optionally substituted by one or more substituents independently selected from halo, oxo, —CN, —OR^{O1}, —NR^{1a}R^{1b}, C₁₋₆ alkyl, C₁₋₄ haloalkyl, 3 to 7 membered monocyclic carbocyclyl and 4 to 7 membered monocyclic heterocyclyl;
each R⁸ is independently halo, oxo, —CN, —OR^{O1}, C₁₋₆ alkyl, C₁₋₄ haloalkyl, phenyl, 5 to 6 membered heteroaryl, 3 to 7 membered monocyclic carbocyclyl or 4 to 7 membered monocyclic heterocyclyl;
each R⁹ is independently halo, oxo, —OR^{O1}, —NR^{N1}R^{N2}, —CN, —C(O)—OR^{O3}, —SO₂—R¹⁰, C₁₋₆ alkyl, phenyl, 5 to 6 membered heteroaryl, 3 to 7 membered monocyclic carbocyclyl or 4 to 7 membered monocyclic heterocyclyl, wherein the C₁₋₆ alkyl, phenyl, 5 to 6 membered heteroaryl, 3 to 7 membered monocyclic carbocyclyl and 4 to 7 membered monocyclic heterocyclyl represented by R⁹ are each optionally substituted by one or more substituents independently selected from halo, oxo, —CN, —OR^{O1}, —NR^{N1}R^{N2}, C₁₋₆ alkyl, C₁₋₄ haloalkyl, phenyl, 5 to 6 membered heteroaryl, 3 to 7 membered monocyclic carbocyclyl and 4 to 7 membered monocyclic heterocyclyl;
each R^{O1} is independently H, C₁₋₆ alkyl, phenyl, 5 to 6 membered heteroaryl, 3 to 7 membered monocyclic or bicyclic carbocyclyl, or 4 to 7 membered monocyclic or bicyclic heterocyclyl, wherein the C₁₋₆ alkyl, phenyl, 5 to 6 membered heteroaryl, 3 to 7 membered monocyclic or bicyclic carbocyclyl, and 4 to 7 membered monocyclic or bicyclic heterocyclyl represented by R^{O1} are each optionally substituted by one or more R^{O2};
each R^{O2} is independently halo, OH, —CN, C₁₋₄ alkoxy, C₁₋₄ alkyl, phenyl, 5 to 6 membered heteroaryl, 3 to 7 membered monocyclic carbocylyl or 4 to 7 membered monocyclic or bicyclic heterocyclyl, wherein the C₁₋₄ alkyl, phenyl, 5 to 6 membered heteroaryl, 3 to 7 membered monocyclic carbocylyl and 4 to 7 membered monocyclic or bicyclic heterocyclyl are each optionally substituted with one or more halo, C₁₋₆ alkyl or —O—C₁₋₆alkyl;
each R^{O3} is independently H, C₁₋₆ alkyl, C₁₋₄ haloalkyl, phenyl, 5 to 6 membered heteroaryl, 3 to 7 membered monocyclic carbocyclyl or 4 to 7 membered monocyclic heterocyclyl, wherein the C₁₋₆ alkyl, phenyl, 5 to 6 membered heteroaryl, 3 to 7 membered monocyclic carbocyclyl and 4 to 7 membered monocyclic heterocyclyl represented by R^{O3} are each optionally substituted by one or more R^{O2};
R^{O4} is H, C₁₋₆ alkyl, phenyl, 5 to 6 membered heteroaryl, 3 to 7 membered monocyclic carbocyclyl or 4 to 7 membered monocyclic heterocyclyl, wherein the C₁₋₆ alkyl, phenyl, 5 to 6 membered heteroaryl, 3 to 7 membered monocyclic carbocyclyl and 4 to 7 membered monocyclic heterocyclyl represented by R^{O4} are each optionally substituted by one or more substituents independently selected from halo, oxo, —CN, —OR^{O1}, —NR^{N1}OR^{N2}, C₁₋₆ alkyl, C₁₋₄ haloalkyl, phenyl, 5 to 6 membered heteroaryl, 3 to 7 membered monocyclic carbocyclyl and 4 to 7 membered monocyclic heterocyclyl;
R^{N1} and R^{N2} are each independently H, C₁₋₆ alkyl or C₃₋₆ cycloalkyl, wherein the C₁₋₆alkyl represented by R^{N1} and R^{N2} are each optionally substituted with C₁₋₄alkoxy;
each R^{N3} is independently H or C₁₋₆ alkyl; and
each R^{N4} is independently H or C₁₋₆ alkyl.

3. The compound of claim 1 or 2, or a pharmaceutically acceptable salt thereof, wherein R^{1A} is H or —CH₃.

4. The compound of claim 1 or 2, wherein the compound is represented by formula (II), (III), (IV), (V) or (VI): 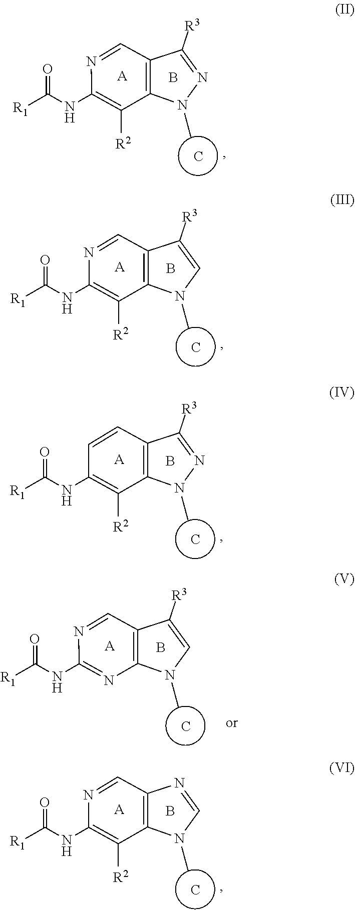
or a pharmaceutically acceptable salt thereof.

5. The compound of any one of claims 1-4, or a pharmaceutically acceptable salt thereof, wherein R² is H or F.

6. The compound of any one of claims 1-4, or a pharmaceutically acceptable salt thereof, wherein R² is H.

7. The compound of any one of claims 1-6, or a pharmaceutically acceptable salt thereof, wherein ring C is phenyl, 5 or 6 membered monocyclic heterocyclyl, or 5 to 6 membered heteroaryl, each of which is optionally substituted by one to three R^{C}.

8. The compound of any one of claims 1-6, or a pharmaceutically acceptable salt thereof, wherein ring C is phenyl or 5 to 6 membered heteroaryl, each of which is optionally substituted by one to three R^{C}.

9. The compound of any one of claims 1-6, or a pharmaceutically acceptable salt thereof, wherein ring C is selected from pyridinonyl, pyridazinonyl, pyrazinonyl, imidazolyl, oxadiazolyl, oxazolyl, phenyl, pyrazinyl, pyridinyl, pyrimidinyl, thiadiazolyl, thiazolyl and triazinyl, each of which is optionally substituted by one or three R^{C}.

10. The compound of any one of claims 1-6, or a pharmaceutically acceptable salt thereof, wherein ring C is selected from imidazolyl, oxadiazolyl, oxazolyl, phenyl, pyrazinyl, pyridinyl, pyrimidinyl, thiadiazolyl, thiazolyl and triazinyl, each of which is optionally substituted by one or three R^{C}.

11. The compound of any one of claims 1-6, or a pharmaceutically acceptable salt thereof, wherein ring C is selected from imidazolyl, oxadiazolyl, oxazolyl, pyrazinyl, pyrimidinyl, thiadiazolyl, thiazolyl and triazinyl, each of which is optionally substituted by one or three R^{C}.

12. The compound of any one of claims 1-6, or a pharmaceutically acceptable salt thereof, wherein ring C is selected from pyrazinyl, pyrimidinyl and thiazolyl, each of which is optionally substituted by one or three R^{C}.

13. The compound of any one of claims 1-6, or a pharmaceutically acceptable salt thereof, wherein ring C is selected from: 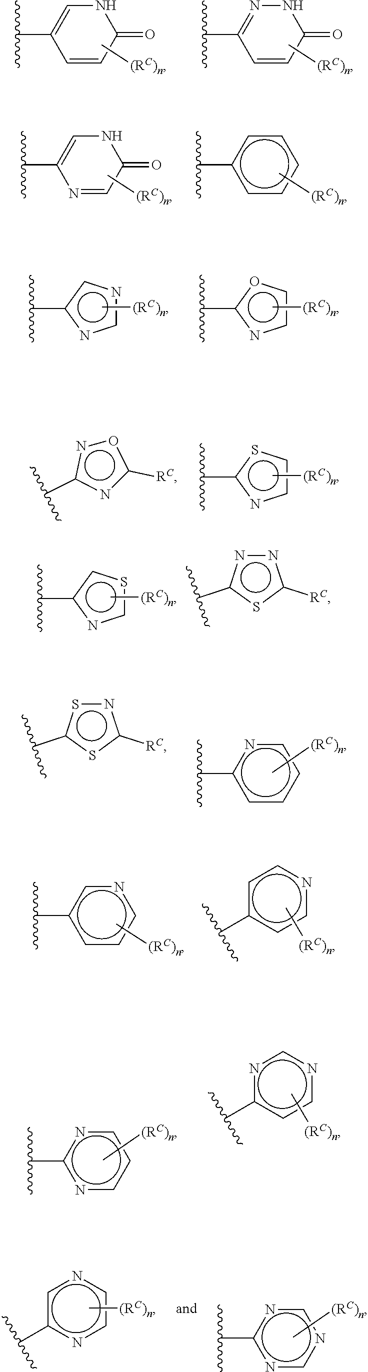
and wherein 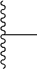
represents a bond to ring B, and n is 0, 1, 2, or 3.

14. The compound of any one of claims 1-6, or a pharmaceutically acceptable salt thereof, wherein ring C is selected from: 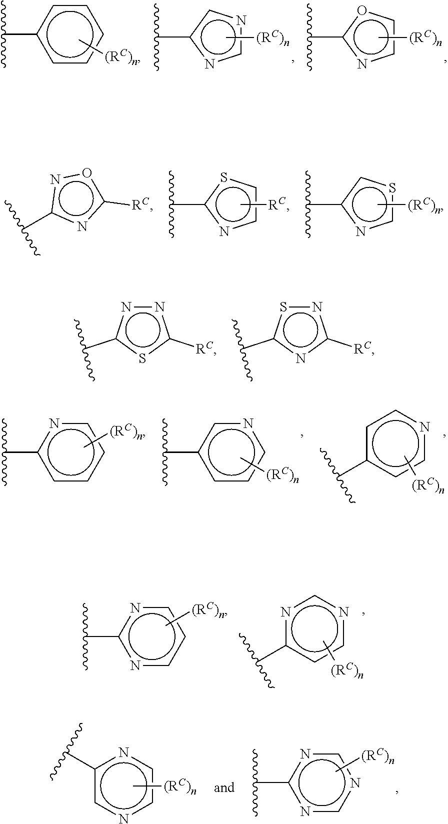
and wherein 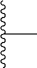
represents a bond to ring B, and n is 0, 1, 2, or 3.

15. The compound of claim 13 or 14, or a pharmaceutically acceptable salt thereof, wherein ring C is selected from:

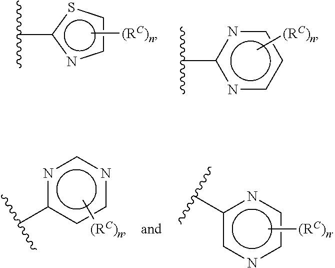

16. The compound of any one of claims 1-6, or a pharmaceutically acceptable salt thereof, wherein ring C is selected from: 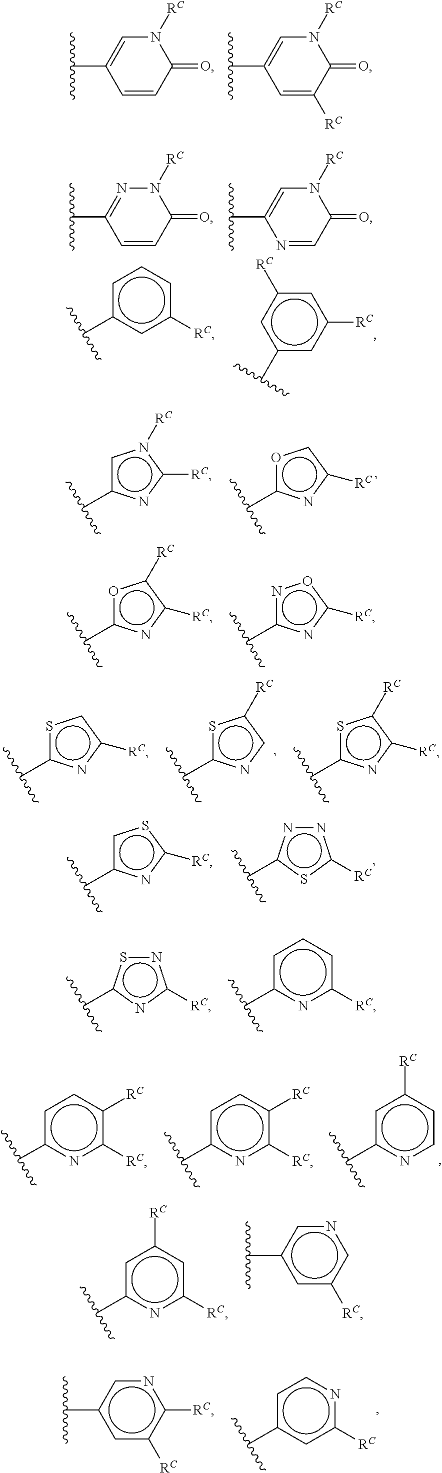 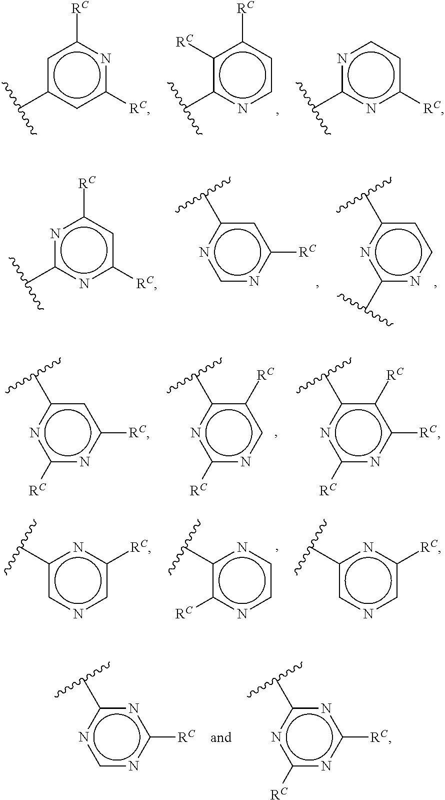
and wherein 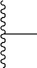
represents a bond to ring B, and two R^{C} groups in ring C may be the same or different.

17. The compound of any one of claims 1-6, or a pharmaceutically acceptable salt thereof, wherein ring C is selected from: 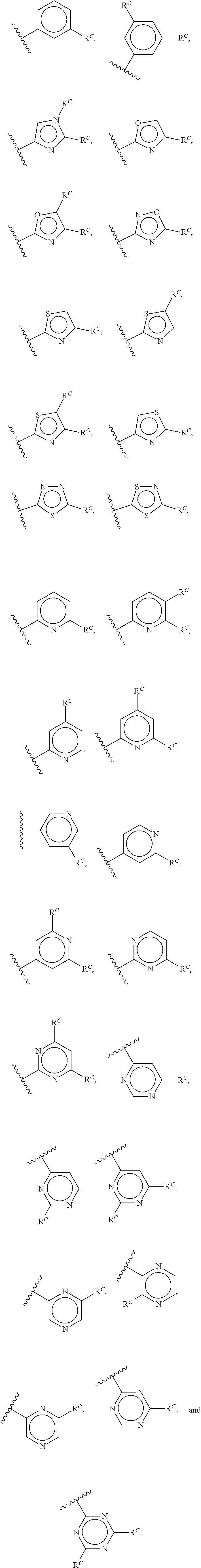
and wherein 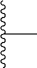
represents a bond to ring B, and two R^{C} groups in ring C may be the same or different.

18. The compound of claim 16 or 17, or a pharmaceutically acceptable salt thereof, wherein ring C is selected from:

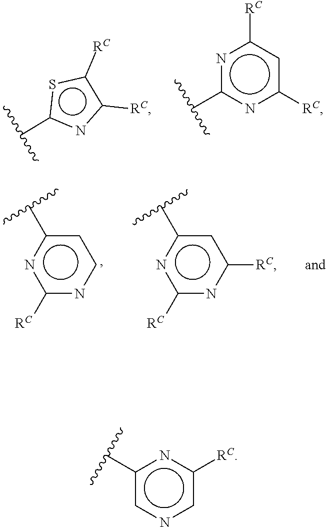

19. The compound of any one of claims 1-18, or a pharmaceutically acceptable salt thereof, wherein each R^{C} is independently halo, —NR^{N1}R^{N2}, —NR^{N3}—C(O)—R⁷, —NR^{N4}—SO₂—R⁷, —C(O)—R⁷, —OR^{O1}, C₁₋₆ alkyl, alkenyl, C₃₋₆ cycloalkyl, phenyl, 5 to 12 membered monocyclic or bicyclic heteroaryl, or 4 to 8 membered monocyclic or bicyclic heterocyclyl, or two R^{C} taken together with intervening atoms form a 3 to 7 membered monocyclic carbocyclyl optionally substituted with one or two halo; wherein the C₁₋₆ alkyl, C₃₋₆ cycloalkyl, 5 to 12 membered monocyclic or bicyclic heteroaryl, and 4 to 8 membered monocyclic or bicyclic heterocyclyl represented by R^{C} are each optionally substituted with one to three R^{C1}.

20. The compound of any one of claims 1-18, or a pharmaceutically acceptable salt thereof, wherein each R^{C} is independently halo, —NR^{N1}R^{N2}, —NR^{N3}—C(O)—R⁷, —NR^{N4}—SO₂—R⁷, —C(O)—R⁷, —OR^{O1}, C₁₋₆ alkyl, C₃₋₆ cycloalkyl, or 4 to 8 membered monocyclic or bicyclic heterocyclyl, wherein the C₁₋₆ alkyl, C₃₋₆ cycloalkyl and 4 to 8 membered monocyclic or bicyclic heterocyclyl represented by R^{C} are each optionally substituted with one to three R^{C1}.

21. The compound of claim 19 or 20, or a pharmaceutically acceptable salt thereof, wherein at least one of R^{C} is C₁₋₃haloalkyl.

22. The compound of claim 21, or a pharmaceutically acceptable salt thereof, wherein at least one of R^{C} is —CF₂CH₃.

23. The compound of claim 19, or a pharmaceutically acceptable salt thereof, wherein the C₃₋₆ cycloalkyl represented by R^{C} is selected from cyclobutyl, cyclopentyl, cyclopropyl and cyclohexyl, the 5 to 12 membered monocyclic or bicyclic heteroaryl represented by R^{C} is furanyl, pyrazoyl, imidazoyl, triazoyl, isoxazole, pyridinyl, pyrimidinyl, isoindolinyl, 3H-imidazo[4,5-b]pyridinyl, 1H-benzo[d][1,2,3]triazolyl, and the 4 to 8 membered monocyclic or bicyclic heterocyclyl represented by R^{C} is selected from azetidinyl, 2,6-diazaspiro[3.3]heptanyl, isothiazolidinyl, isothiazolidinedioxide, morpholinyl, oxabicycloheptanyl, oxetanyl, piperidinyl, piperizinyl, pyrrolidinyl, pyrrolidinonyl, tetrahydrofuranyl, pyridin-2(1H)-oyl, tetrahydro-2H-pyranyl, 2-oxabicyclo[2.1.1]hexanyl, 2-oxa-6-azaspiro[3.4]octanyl and 7-oxabicyclo[2.2.1]heptanyl, wherein each of the C₃₋₆ cycloalkyl, 5 to 12 membered monocyclic or bicyclic heteroaryl, and 4 to 8 membered monocyclic or bicyclic heterocyclyl is optionally substituted with one to three R^{C1}, or two R^{C} taken together with intervening atoms form cyclopentyl substituted with one or two halo.

24. The compound of claim 19, or a pharmaceutically acceptable salt thereof, wherein the C₃₋₆ cycloalkyl and the 4 to 8 membered monocyclic or bicyclic heterocyclyl represented by R^{C} is represented by the following formula: 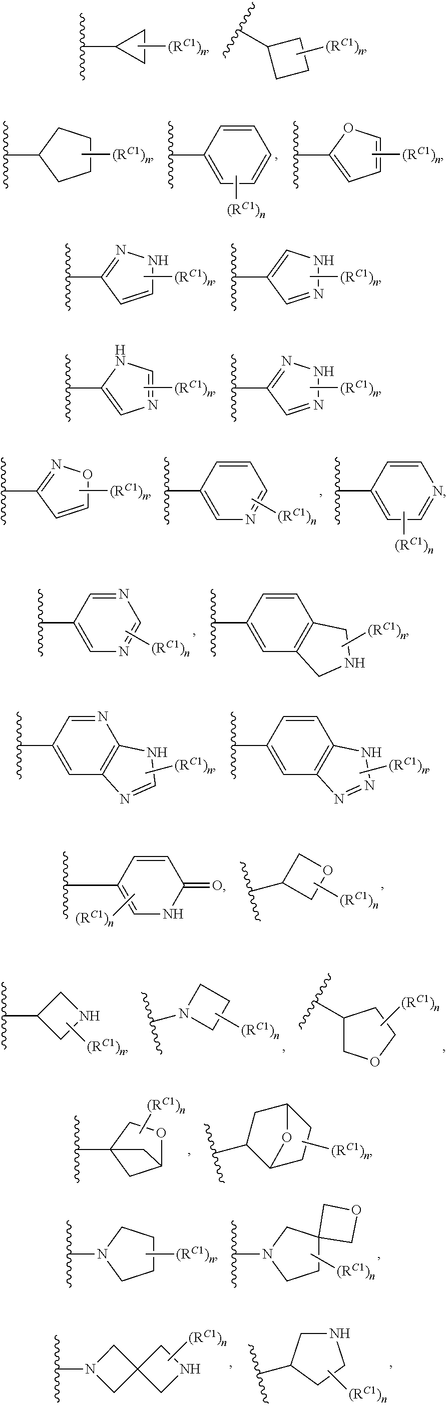 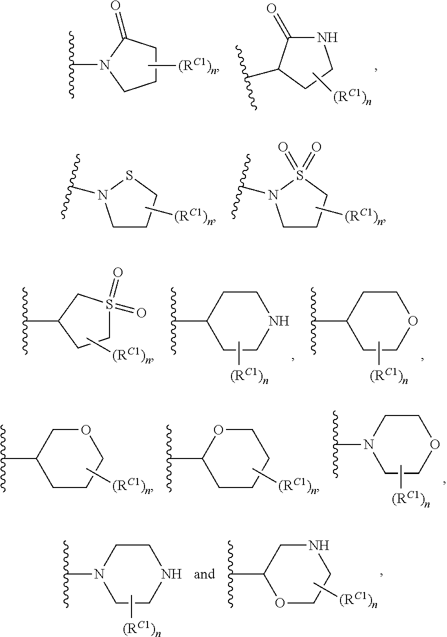
wherein 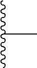
represents a bond to ring C, and n is 0, 1, 2 or 3.

25. The compound of claim 20, or a pharmaceutically acceptable salt thereof, wherein the C₃₋₆ cycloalkyl and the 4 to 8 membered monocyclic or bicyclic heterocyclyl represented by R^{C} is represented by the following formula: 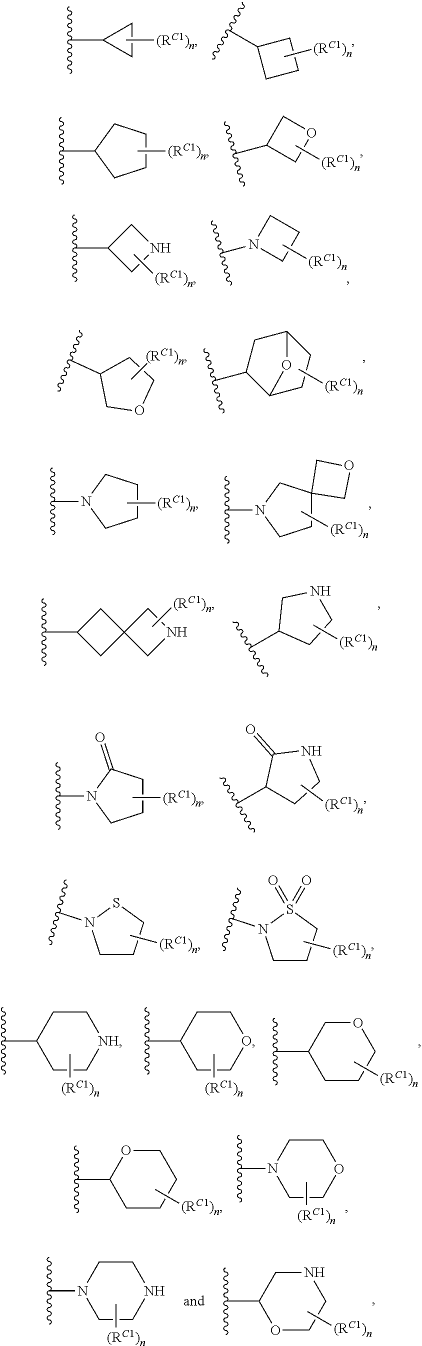
wherein 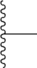
represents a bond to ring C, and n is 0, 1, 2 or 3.

26. The compound of claim 19, or a pharmaceutically acceptable salt thereof, wherein the C₃₋₆ cycloalkyl and the 4 to 8 membered monocyclic or bicyclic heterocyclyl represented by R^{C} is independently selected from: 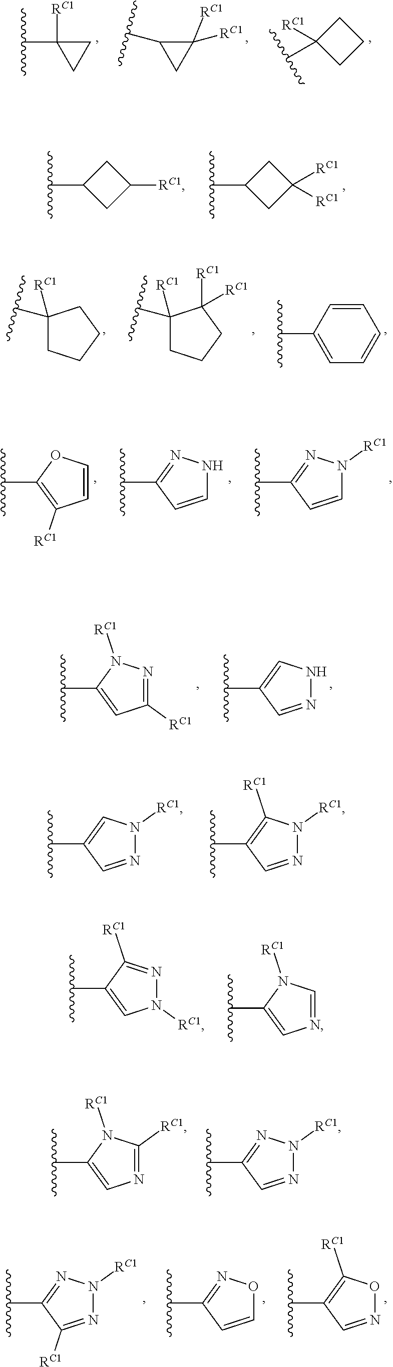 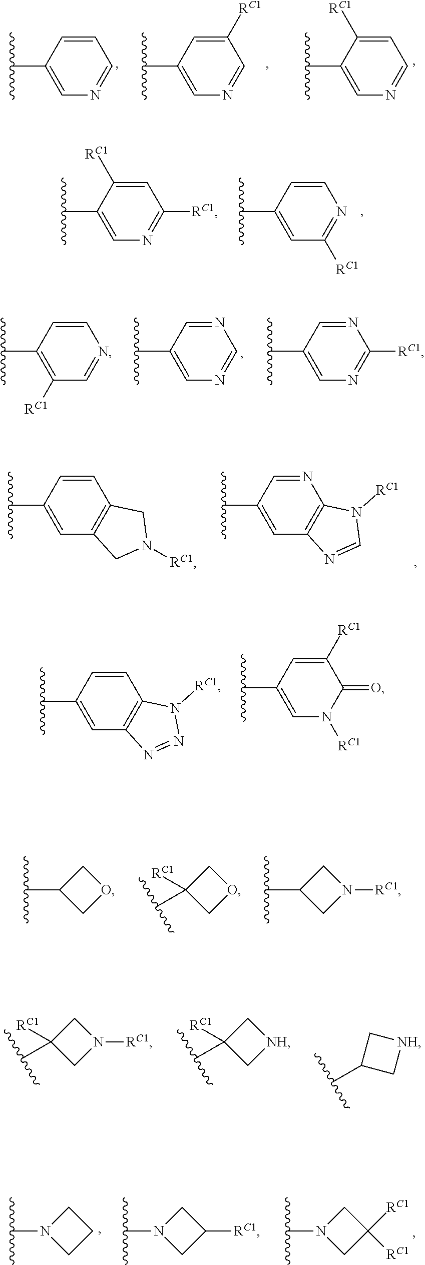 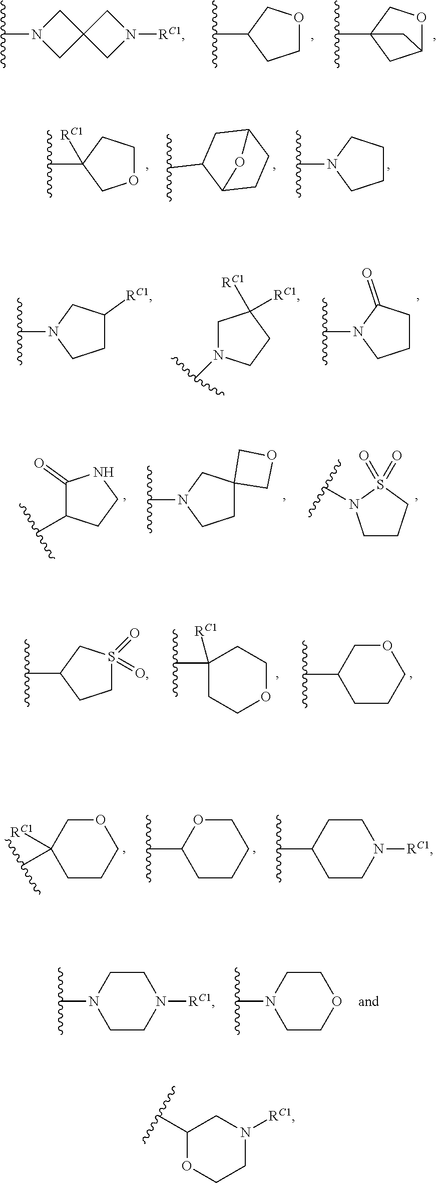
wherein 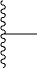
represents a bond ring C, and two R^{C1} groups may be the same or different.

27. The compound of claim 20, or a pharmaceutically acceptable salt thereof, wherein the C₃₋₆ cycloalkyl and the 4 to 8 membered monocyclic or bicyclic heterocyclyl represented by R^{C} is independently selected from: 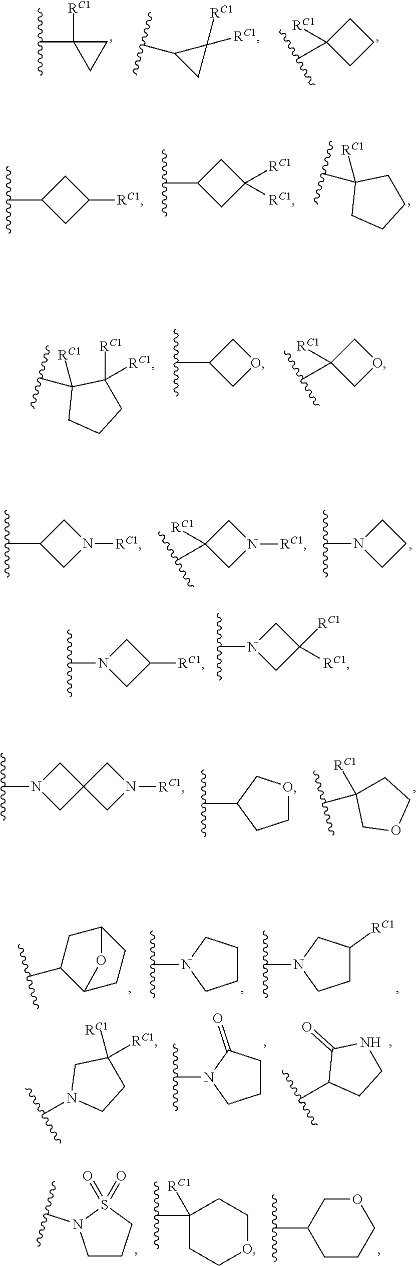 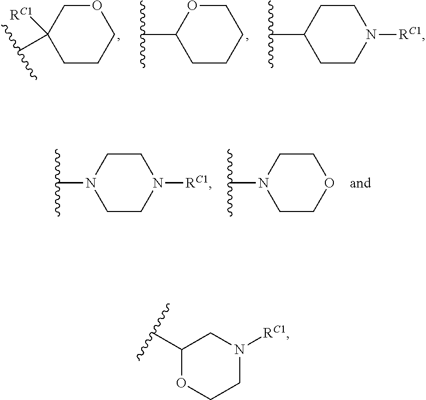
wherein 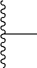
represents a bond ring C, and two R^{C1} groups may be the same or different.

28. The compound of any one of claims 1-27, or a pharmaceutically acceptable salt thereof, wherein
each R^{C1} is independently halo, —CN, —OR^{O1}, —NR^{N1}R^{N2}, —C(O)—R⁷, —C(O)—OR³, —SO₂—R⁷, C₁₋₆ alkyl, C₃₋₆cycloalkyl, or 4 to 6 membered monocyclic heterocyclyl, wherein the C₁₋₆ alkyl represented by R^{C1} is optionally substituted by one to three substituents independently selected from halo and —OR^{a1}; and
R^{a1} is H, C₁₋₄ alkyl or 4 to 6 membered heterocyclyl.

29. The compound of any one of claims 1-27, or a pharmaceutically acceptable salt thereof, wherein
each R^{C1} is independently halo, —CN, —OR^{O1}, —NR^{N1}R^{N2}, —C(O)—R⁷, —C(O)—OR^{O3}, —SO₂—R⁷, C₁₋₆ alkyl or 4 to 6 membered monocyclic heterocyclyl, wherein the C₁₋₆ alkyl represented by R^{C1} is optionally substituted by one to three substituents independently selected from halo and —OR^{a1}; and
R^{a1} is H, C₁₋₄ alkyl or 4 to 6 membered heterocyclyl.

30. The compound of claim 28, or a pharmaceutically acceptable salt thereof, wherein each R^{C1} is independently selected from F, Cl, —CN, OH, —OCH₃, —OCHF₂, —NH₂, —N(CH₃)₂, —CH₃, —CH₂F, —CHF₂, —CF₃, —CH₂—OH, —CH₂—CH₃, —CH(CH₃)₂, —CH₂—O—CH₃, —CH₂—CH₂—OCH₃, —C(O)—CH₃, —C(O)—OC(CH₃)₃, —SO₂—CH₃, cyclopropyl, 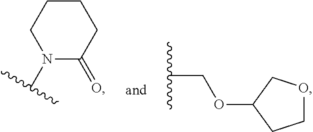 [SMILES: *N1CCCCC1=O ; "and" ; *COC1CCOC1]
wherein 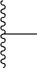
represents a bond to R^{C}.

31. The compound of claim 29, or a pharmaceutically acceptable salt thereof, wherein each R^{C1} is independently selected from F, —CN, OH, —OCH₃, —OCHF₂, —NH₂, —N(CH₃)₂, —CH₃, —CH₂F, —CHF₂, —CF₃, —CH₂—OH, —CH₂—CH₃, —CH(CH₃)₂, —C(O)—CH₃, —C(O)—OC(CH₃)₃, —SO₂—CH₃, 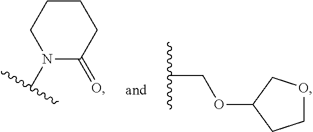 [SMILES: *N1CCCCC1=O ; "and" ; *COC1CCOC1]
wherein 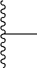
represents a bond to R^{C}.

32. The compound of claim 19, or a pharmaceutically acceptable salt thereof, wherein each R^{C} is independently selected from —F, —Cl, —CH₃, —CH₂F, —CHF₂, —CF₃, —CH₂—CH₃, —CH₂—CH₂F, —CH₂—CHF₂, —CH₂—CF₃, —CHF—CH₂F, —CH₂—CHF₂, —CH₂—CH₂—CH₂F, —CH₂—CF₃, —C(CH₃)₃, —CF₂CH₃, —CHF—CH₃, —CH(CH₃)₂, —CF(CH₃)₂, —C(CH₃)₂—CH₂F, —CH(CH₃)—CHF₂, —CH(CH₃)—CF₃, —C(CH₃)₂—CF₃, —CH₂—CH₂—CN, —CH₂OCH₃, —CH₂—C(CH₃)₂—CN, —C(CH₃)₂—OH, —CH(CH₃)—OCH₃, —C(CH₃)₂—OCH₃, —C(CH₃)₂—CH₂—OCH₃, —CH(OH)—CH₃, —C(CH₃)(OH)—CF₃, —CH═CH₂, —OCH₃, —O—CHF₂, —C(CH₃)(OCH₃)—CH₂—OCH₃, —O—CF₃, —CH(OCH₃)-cyclopropyl, —OH, —O—CH₂CH₃, —O—CH₂CHF₂, —O—CH(CH₃)₂, —O—CH(CF₃)₂, —O—CH₂—OCH₃, —O—CH(CH₃)—OCH₃, —O—CH(CH₃)—CH₂—OCH₃, —O—CH₂—CH₂—O—CH₃, —O—CH₂—CH(CH₃)—OCH₃, —CF(CH₃)—CH₂—OCH₃, —CH(CF₃)—NH₂, —CH(OCH₃)—C(CH₃)₃, —NH—C(O)—CH₃, —NH—SO₂—CH₃, —NH₂, —NHCH₃, —N(CH₃)₂, —NHCH(CH₃)₂, —NHCH₂CH₂CH₃, —NHCH₂C(CH₃)₂OCH₃, cyclopropyl, —CHF-cyclopropyl, —CF(CH₃)-cyclopropyl, —CH(OCH₃)-cyclopropyl, —C(CH₃)(OCH₃)-cyclopropyl, —CF(OCH₃)-cyclopropyl, —NH-cyclohexyl, —NH-cyclopropyl, —NH—N-methylpiperidine, —NH—CH₂-cyclopropyl, —NH—CH₂—CH₂—OCH₃, —N(CH₃)—CH₂—CH₂—OCH₃, —CH(NH₂)—CF₃, 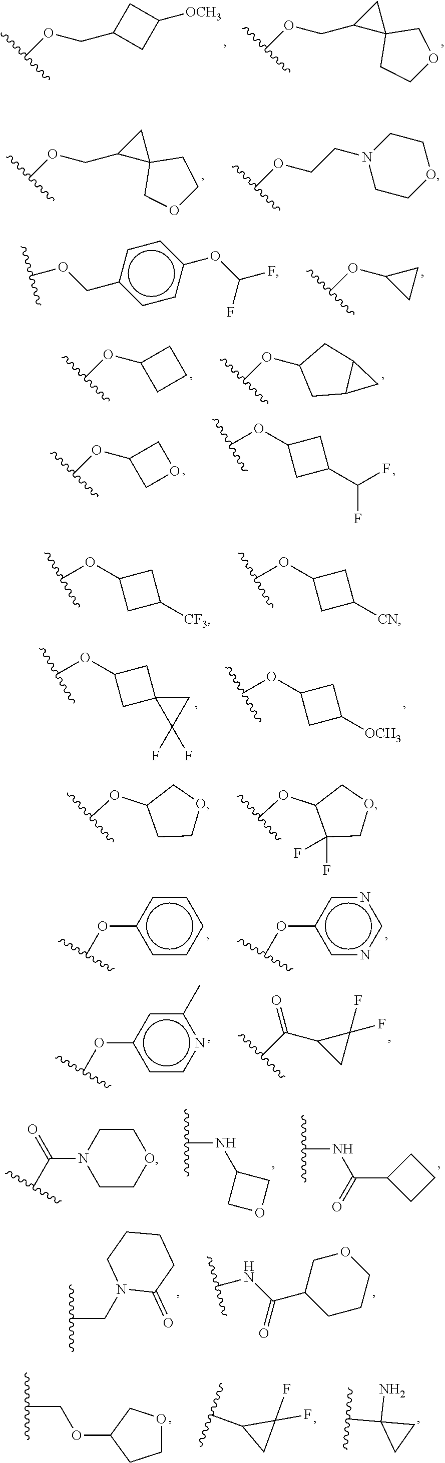 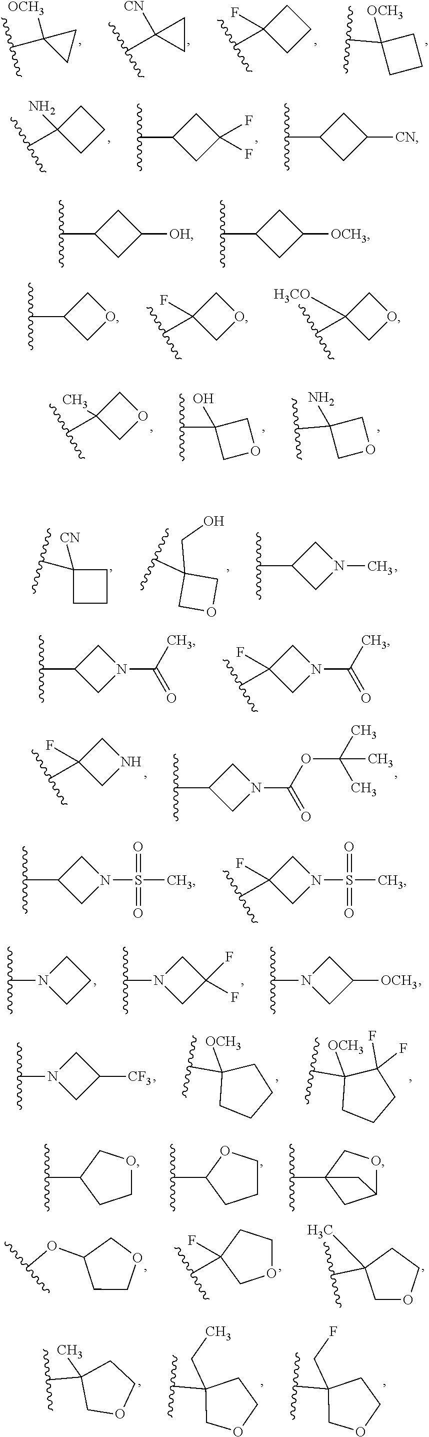 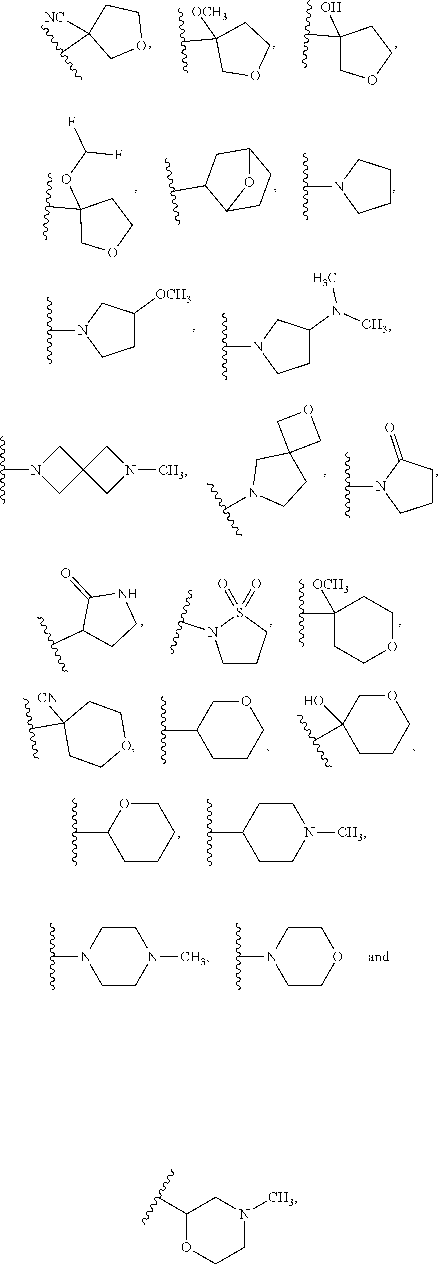
wherein 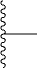
represents a bond to ring C.

33. The compound of claim 20, or a pharmaceutically acceptable salt thereof, wherein each R^{C} is independently selected from Cl, —CH₃, —CH₂F, —CHF₂, —CF₃, —CH₂—CH₃, —CH₂—CH₂F, —CH₂—CHF₂, —CH₂—CF₃, —CHF—CH₂F, —CH₂—CHF₂, —CH₂—CF₃, —C(CH₃)₃, —CF₂CH₃, —CHF—CH₃, —CH(CH₃)₂, —CF(CH₃)₂, —C(CH₃)₂—CH₂F, —CF(CH₃)₂, —CH(CH₃)—CHF₂, —CH(CH₃)—CF₃, —C(CH₃)₂—CF₃, —CH₂—CH₂—CN, —CH₂—C(CH₃)₂—CN, —C(CH₃)₂—OH, —CH(CH₃)—OCH₃, —C(CH₃)₂—OCH₃, —CH(OH)—CH₃, —OCH₃, —O—CHF₂, —C(CH₃)(OCH₃)—CH₂—OCH₃, —O—CF₃, —CH(OCH₃)-cyclopropyl, —O—CH₂CH₃, —O—CH₂CHF₂, —O—CH(CH₃)₂, —O—CH(CF₃)₂, —O—CH₂—OCH₃, —O—CH(CH₃)—OCH₃, —O—CH(CH₃)—CH₂—OCH₃, —O—CH₂—CH₂—O—CH₃, —O—CH₂—CH(CH₃)—OCH₃, —CF(CH₃)—CH₂—OCH₃, —CH(CF₃)—NH₂, —CH(OCH₃)—C(CH₃)₃, —NH—C(O)—CH₃, —NH—SO₂—CH₃, —N(CH₃)₂, —NHCH(CH₃)₂, —CHF-cyclopropyl, —CF(CH₃)-cyclopropyl, —NH-cyclohexyl, —N(CH₃)—CH₂—CH₂—OCH₃, —CH(NH₂)—CF₃, 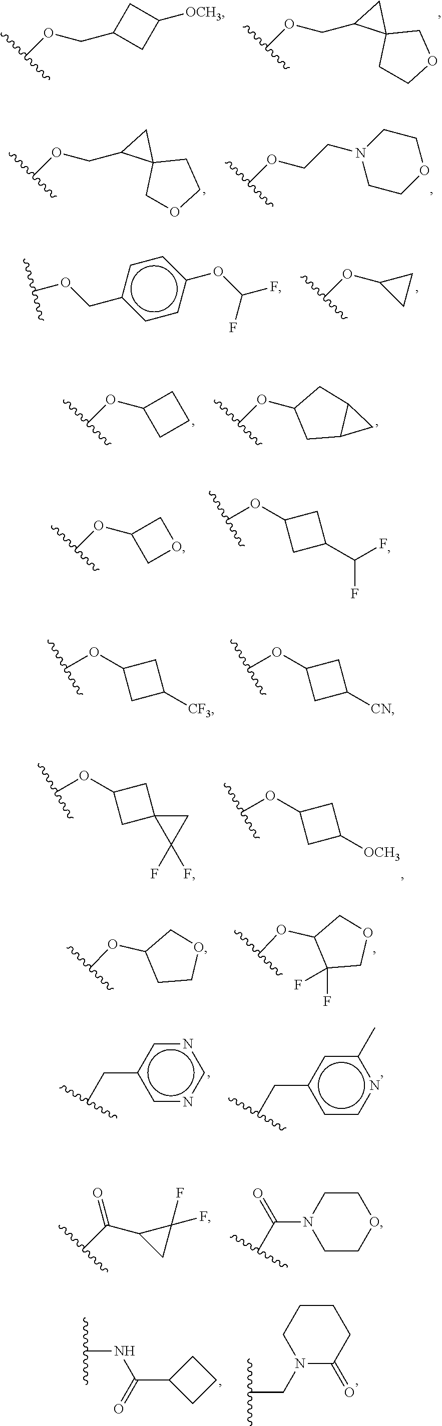 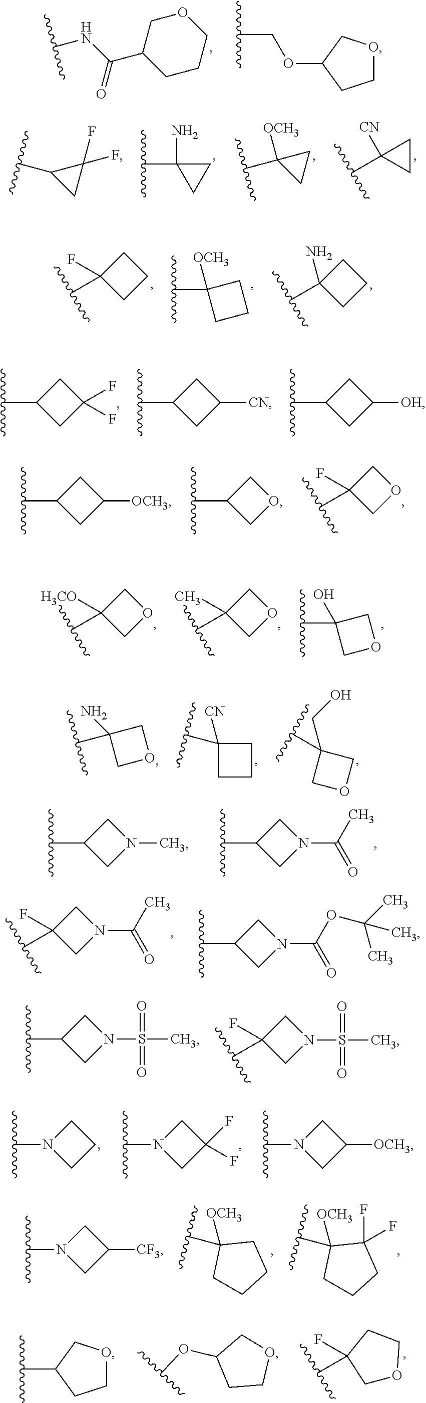 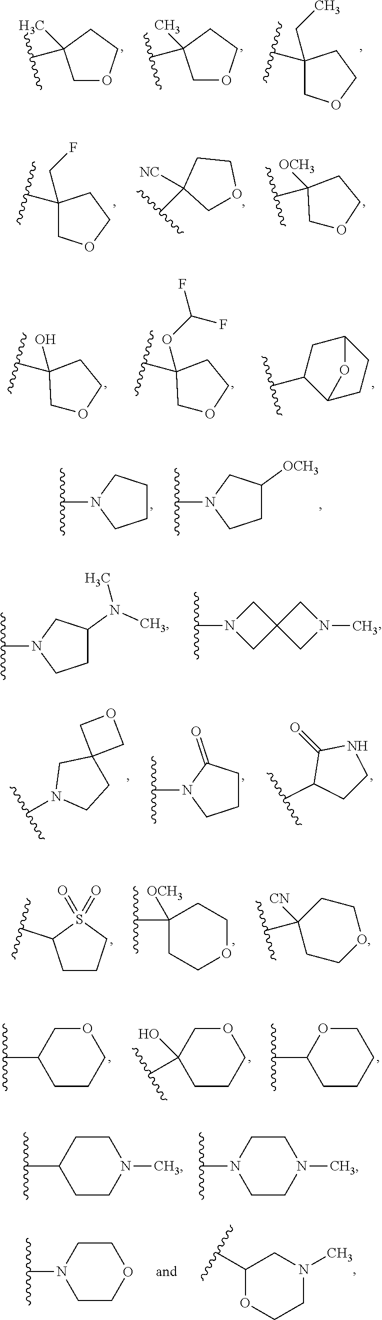
wherein 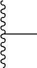
represents a bond to ring C.

34. The compound of any one of claims 1-33, or a pharmaceutically acceptable salt thereof, wherein:
R¹ is H, C₁₋₄ alkyl, —OR^{1A}, —NR^{N1}R^{N2}, or C₃₋₆ cycloalkyl, wherein the C₁₋₄ alkyl and C₃₋₆ cycloalkyl represented by R¹ are each optionally substituted by one to three R⁸ independently selected from halo, —CN, C₁₋₃alkoxy, C₁₋₃alkyl and C₁₋₃haloalkyl;
R^{1A} is C₁₋₄alkyl;
R^{N1} and R^{N2} are each independently H or C₁₋₄alkyl.

35. The compound of any one of claims 1-33, or a pharmaceutically acceptable salt thereof, wherein R¹ is C₁₋₄ alkyl or C₃₋₆ cycloalkyl, wherein the C₁₋₄ alkyl and C₃₋₆ cycloalkyl represented by R¹ are each optionally substituted by one to three R⁸ independently selected from halo, —CN, C₁₋₃alkoxy, C₁₋₃alkyl and C₁₋₃haloalkyl.

36. The compound of any one of claims 1-33, or a pharmaceutically acceptable salt thereof, wherein R¹ is C₁₋₄ alkyl optionally substituted by one to three R independently selected from halo, —CN, C₁₋₃alkoxy, C₁₋₃alkyl and C₁₋₃haloalkyl.

37. The compound of any one of claims 34 to 36, or a pharmaceutically acceptable salt thereof, wherein R⁸, for each occurrence, is independently halo, —CN, C₁₋₃alkoxy.

38. The compound of claim 37, or a pharmaceutically acceptable salt thereof, wherein each R⁸ is independently selected from F, —CN and —OCH₃.

39. The compound of claim 34, or a pharmaceutically acceptable salt thereof, wherein R¹ is selected from —H, —CH₃, —CD₃, —CH₂—CH₃, —CH₂—CHF₂, —CH₂—CH₂—CN, —CH₂—CH₂—OCH₃, —OCH₃, —NH₂, —NHCH₃, —N(CH₃)₂, cyclopropyl

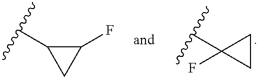 [SMILES: *C1CC1F ; "and" ; *C1(F)CC1 ; "."]

40. The compound of claim 35, or a pharmaceutically acceptable salt thereof, wherein R¹ is selected from —CH₃, —CH₂—CH₃, —CH₂—CHF₂, —CH₂—CH₂—CN, —CH₂—CH₂—OCH₃, cyclopropyl,

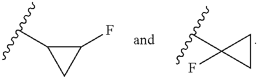 [SMILES: *C1CC1F ; "and" ; *C1(F)CC1 ; "."]

41. The compound of any one of claims 1-40, or a pharmaceutically acceptable salt thereof, wherein R³ is H, —NR^{N1}R^{N2}, halo, —C(O)—R⁷, —C(O)—OR^{O3}, —SO₂—R⁷, —OR^{O4}, C₁₋₆ alkyl, alkenyl C₃₋₆cycloalkyl, 5 or 6 membered heteroaryl, 4 to 10 membered monocyclic or bicyclic heterocyclyl, wherein the C₁₋₆ alkyl, C₃₋₆cycloalkyl, and 4 to 9 membered monocyclic or bicyclic heterocyclyl represented by R³ are each optionally substituted by one to three R⁹.

42. The compound of any one of claims 1-40, or a pharmaceutically acceptable salt thereof, wherein R³ is H, —NR^{N1}R^{N2}, halo, —C(O)—R⁷, —C(O)—OR^{O3}, —SO₂—R⁷, —OR^{O4}, C₁₋₆ alkyl, C₃₋₆cycloalkyl, 4 to 9 membered monocyclic or bicyclic heterocyclyl, wherein the C₁₋₆ alkyl, C₃₋₆cycloalkyl, and 4 to 9 membered monocyclic or bicyclic heterocyclyl represented by R³ are each optionally substituted by one to three R⁹.

43. The compound of claim 41, or a pharmaceutically acceptable salt thereof, wherein R³ is C₃₋₆ cycloalkyl or 4 to 10 membered monocyclic or bicyclic heterocyclyl, each optionally substituted by one or three R⁹.

44. The compound of claim 42, or a pharmaceutically acceptable salt thereof, wherein R³ is C₃₋₆ cycloalkyl or 4 to 9 membered monocyclic or bicyclic heterocyclyl, each optionally substituted by one or three R⁹.

45. The compound of claim 41, or a pharmaceutically acceptable salt thereof, wherein R³ is selected from pyrazoyl, pyridinyl, azetidinyl, cyclobutyl, cyclopentyl, cyclopropyl, 2-oxaspiro[3.3]heptanyl, 1,7-diazaspiro[4.4]nonanyl, 2,7-diazaspiro[3.5]nonanyl, 7-oxa-2-azaspiro[3.5]nonanyl, 1-oxa-7-azaspiro[4.4]nonanyl, 2,6-diazaspiro[3.4]octanyl, 2-oxa-6-azaspiro[3.4]octanyl, 6-oxa-2-azaspiro[3.4]octanyl, 2,7-diazaspiro[4.4]nonanyl, 1,6-diazaspiro[3.3]heptanyl, 1-oxa-6-azaspiro[3.3]heptanyl, 3,6-diazabicyclo[3.2.0]heptanyl, 3,9-diazabicyclo[3.3.1]nonanyl, 6-oxa-2,9-diazaspiro[4.5]decanyl, 1,6-diazaspiro[3.4]octanyl, 5-azaspiro[2.4]heptanyl, 1,6-diazaspiro[3.4]octanyl, 1,7-diazaspiro[4.4]nonanyl, 2-oxa-7-azaspiro[4.4]nonanyl, octahydropyrano[2,3-c]pyrrolyl, octahydro-1H-pyrrolo[3,4-b]pyridinyl, octahydropyrrolo[3,4-b][1,4]oxazinyl, octahydropyrrolo[3,4-b]pyrrolyl, 3-oxa-6-azabicyclo[3.1.1]heptanyl, hexahydro-1H-furo[3,4-c]pyrrolyl, 1,4-oxazepanyl, 6-oxa-2-azaspiro[3.5]nonanyl, 5-oxa-2-azaspiro[3.4]octanyl, 8-oxa-3-azabicyclo[3.2.1]octanyl, 3-oxa-7-azabicyclo[3.3.1]nonanyl, 2,5-diazabicyclo[2.2.1]heptanyl, 3,6-diazabicyclo[3.1.1]heptanyl, 3,6-diazabicyclo[3.2.0]heptanyl, 2,5-diazabicyclo [2.2.2]octanyl, 3,8-diazabicyclo[3.2.1]octanyl, morpholinyl, octahydropyrrolo[3,4-c]pyrrolyl, 2-oxa-5-azabicylo[2.2.1]heptanyl, 6-oxa-3-azabicyclo[3.1.1]heptanyl, 8-oxa-3-azabicyclo[3.2.1]octanyl, 2λ²,6-diazaspiro[3.3]heptanyl, 1λ²,7λ²-diazaspiro[4.4]nonanyl, oxetanyl, piperidinyl, piperazinyl, piperazine-2-one-yl, pyrrolidinyl, pyrrolidine-2-one-yl and tetrahydropyranyl, each of which is optionally substituted by one to three R⁹.

46. The compound of claim 42, or a pharmaceutically acceptable salt thereof, wherein R³ is selected from azetidinyl, cyclobutyl, cyclopentyl, cyclopropyl, 2,5-diazabicyclo[2.2.1]heptanyl, 3,6-diazabicyclo[3.1.1]heptanyl, 3,6-diazabicyclo[3.2.0]heptanyl, 2,5-diazabicyclo [2.2.2]octanyl, 3,8-diazabicyclo[3.2.1]octanyl, morpholinyl, octahydropyrrolo[3,4-c]pyrrolyl, 2-oxa-5-azabicylo[2.2.1]heptanyl, 6-oxa-3-azabicyclo[3.1.1]heptanyl, 8-oxa-3-azabicyclo[3.2.1]octanyl, 2λ²,6-diazaspiro[3.3]heptanyl, 1λ²,7λ²-diazaspiro[4.4]nonanyl, oxetanyl, piperidinyl, piperazinyl, piperazine-2-one-yl, pyrrolidinyl, pyrrolidine-2-one-yl and tetrahydropyranyl, each of which is optionally substituted by one to three R⁹.

47. The compound claim 41 or 42, or a pharmaceutically acceptable salt thereof, wherein R³ is selected from pyrrolidinyl, morphonlinyl, 6-oxa-3-azabicyclo[3.1.1]heptanyl, and 3,6-diazabicyclo[3.1.1]heptanyl, each of which is optionally substituted by one to three R⁹.

48. The compound claim 41 or 42, or a pharmaceutically acceptable salt thereof, wherein R³ is pyrrolidinyl optionally substituted by one to three R⁹.

49. The compound of any one of claims 1-40, or a pharmaceutically acceptable salt thereof, wherein R³ is selected from: 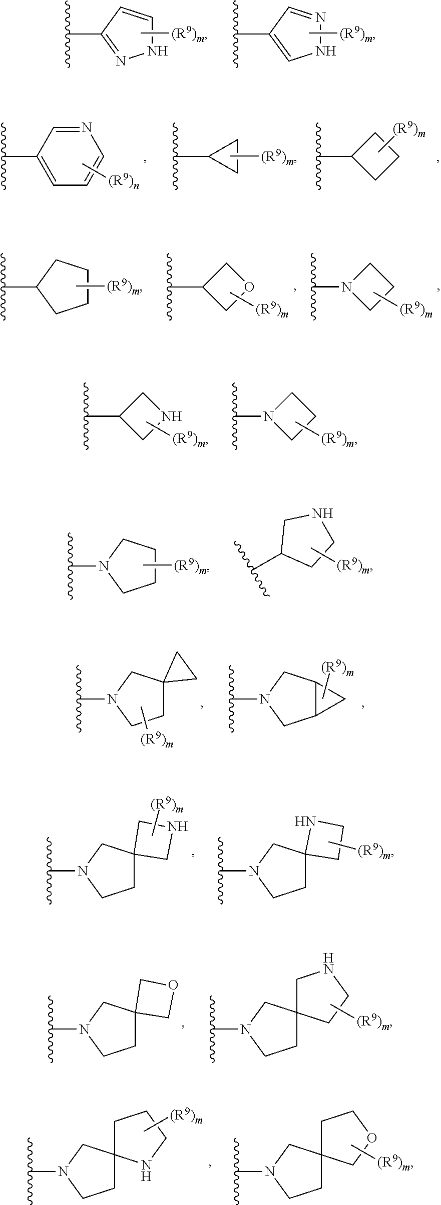 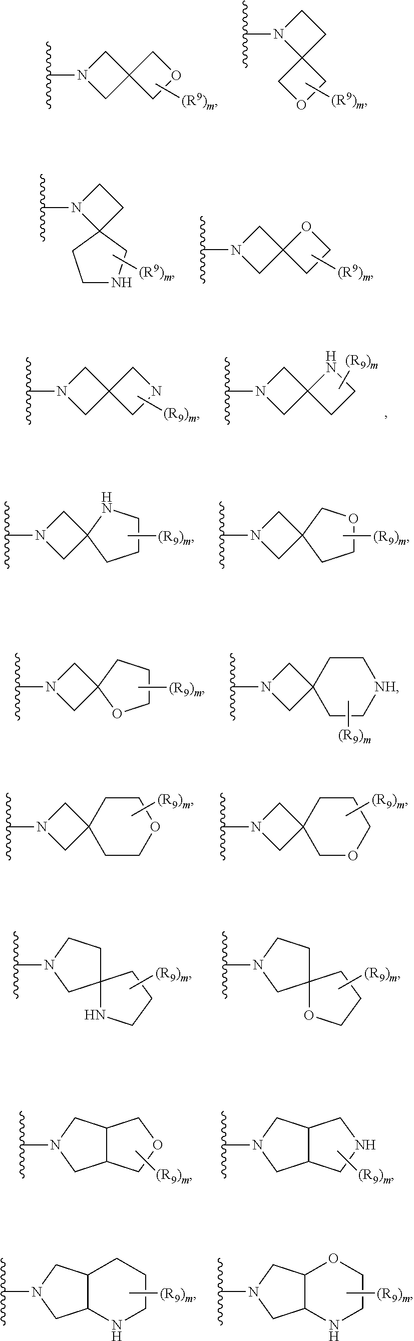 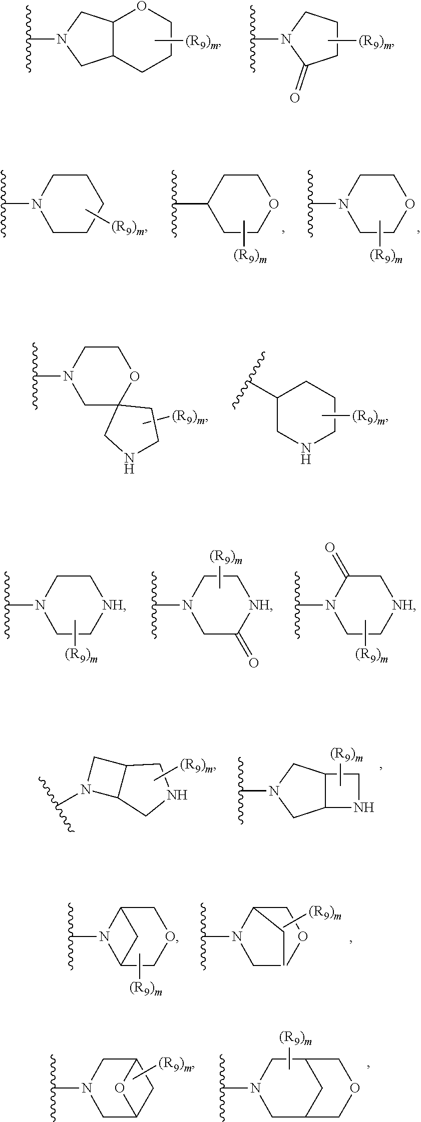 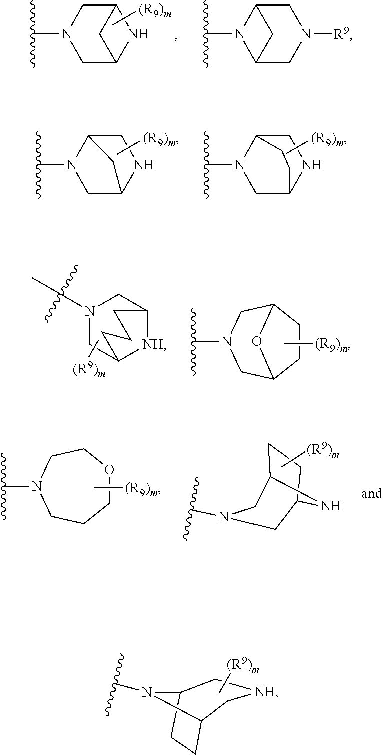
wherein 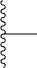
represents a bond to ring B, and m is 0, 1, 2, 3 or 4.

50. The compound of claim 49, or a pharmaceutically acceptable salt thereof, wherein R³ is selected from:

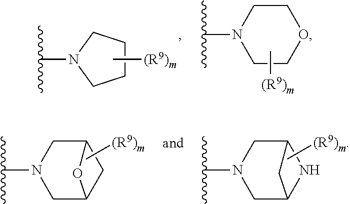

51. The compound of any one of claims 1-40, or a pharmaceutically acceptable salt thereof, wherein R³ is selected from: 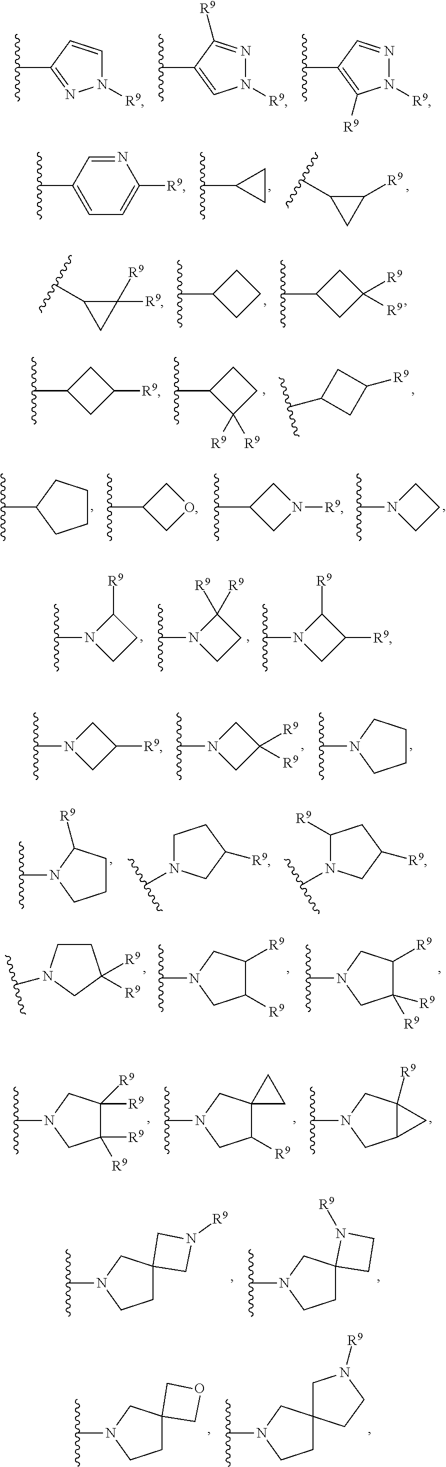 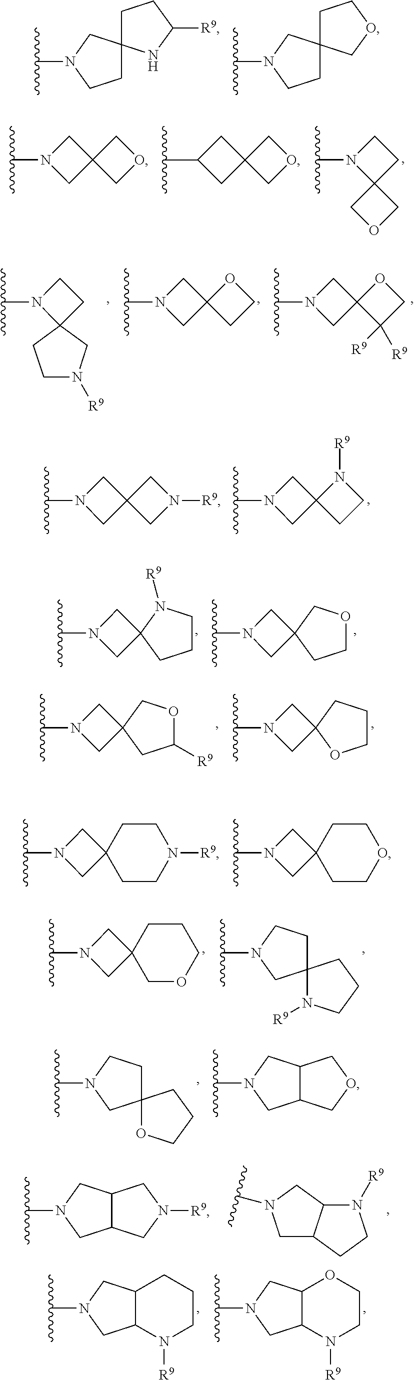 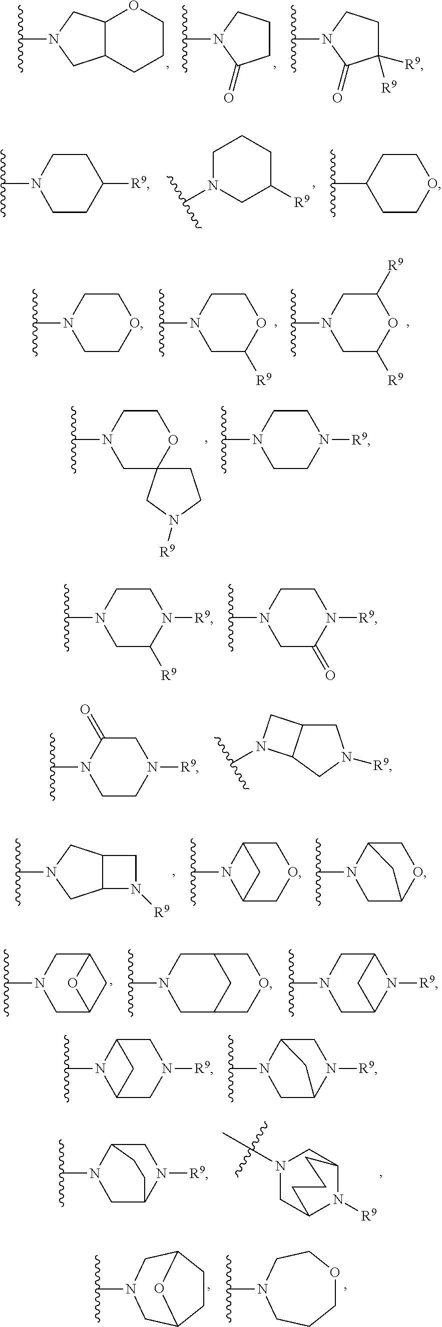 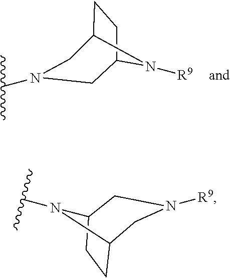
wherein 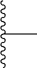
represents a bond to ring B.

52. The compound of any one of claims 1-40, or a pharmaceutically acceptable salt thereof, wherein R³ is selected from: 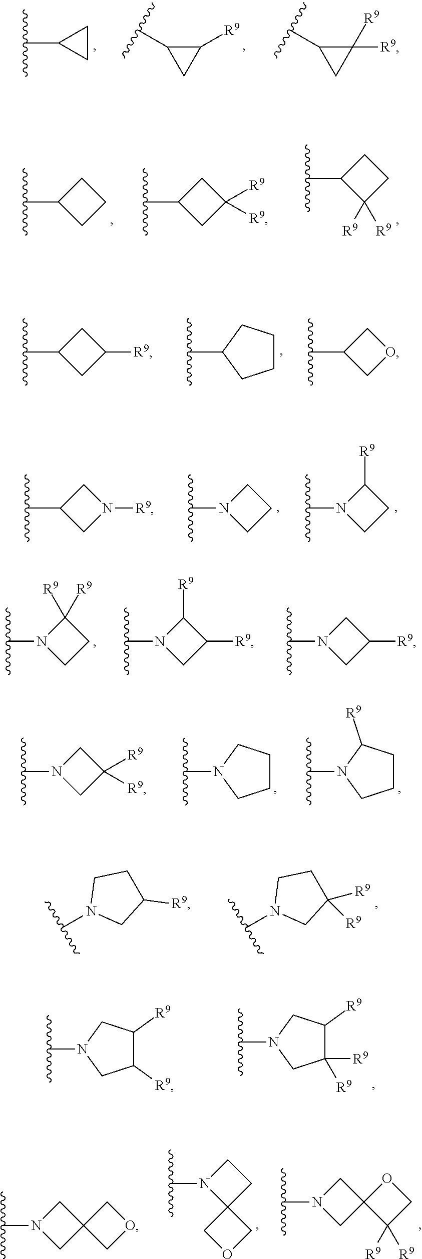 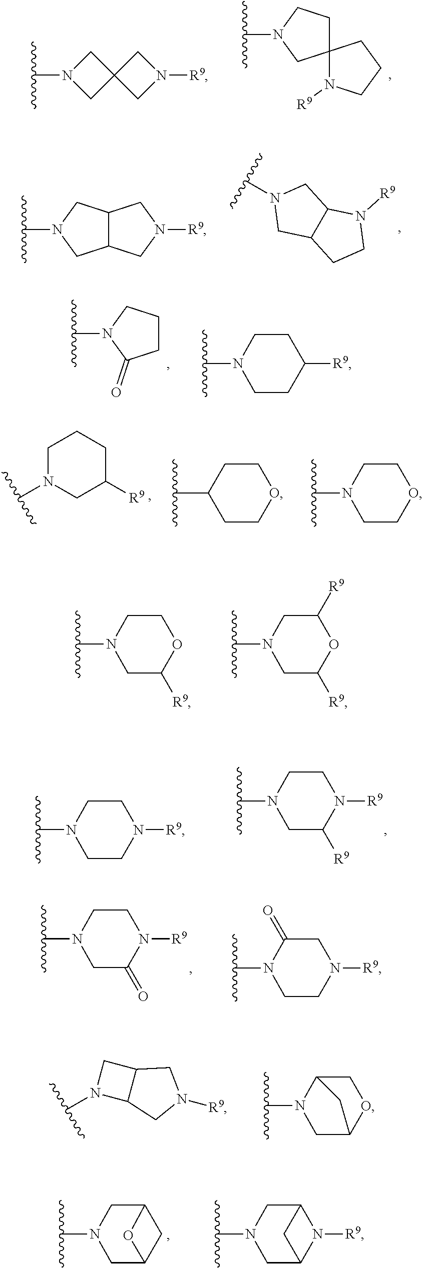 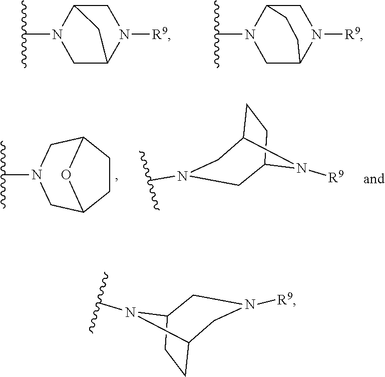
wherein 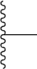
represents a bond to ring B.

53. The compound of any one of claims 1-40, or a pharmaceutically acceptable salt thereof, wherein R³ is H, —NR^{N1}R^{N2}, halo, —C(O)—R⁷, —C(O)—OR^{O3}, —SO₂—R⁷, —OR^{O4}, or C₁₋₆ alkyl optionally substituted with one to three R⁹.

54. The compound of any one of claims 1-53, or a pharmaceutically acceptable salt thereof, wherein each R⁹ is independently halo, OH, —OC₁₋₄alkyl, —NR^{a2}R^{a3}, —CN, —C(O)—OR^{a1}, —SO₂—R^{a1}, C₁₋₄ alkyl, or C₃₋₆ cycloalkyl, wherein the C₁₋₄ alkyl and C₃₋₆ cycloalkyl represented by R⁹ are each optionally substituted by one to three substituents independently selected from halo and C₁₋₃ alkoxy; and R^{a1}, R^{a2} and R^{a3} are each independently C₁₋₃ alkyl.

55. The compound of any one of claims 1-53, or a pharmaceutically acceptable salt thereof, wherein each R⁹ is independently halo, —OR^{a1}, —NR^{a}R^{a3}, —CN, —C(O)—OR^{a1}, —SO₂—R^{a1}, C₁₋₄ alkyl, C₃₋₆ cycloalkyl, or 4 to 10 membered monocyclic or bicyclic heterocyclyl, wherein the C₁₋₄ alkyl, 4 to 10 membered monocyclic or bicyclic heterocyclyl, and C₃₋₆ cycloalkyl represented by R⁹ are each optionally substituted by one to three substituents independently selected from halo, OH, —CN, C₁₋₄ alkyl, and C₁₋₃ alkoxy; and R^{a1}, R^{a2} and R^{a3} are each independently H, C₃₋₄cycloalkyl, or C₁₋₃ alkyl, wherein the C₃₋₄cycloalkyl represented by R^{a1}, R^{a2} and R^{a3} is optionally substituted with C₁₋₄alkyl, and wherein the C₁₋₃ alkyl represented by R^{a1}, R^{a2} and R^{a3} is optionally substituted with phenyl.

56. The compound of any one of claims 1-53, or a pharmaceutically acceptable salt thereof, wherein each R⁹ is independently halo, —OR^{a1}, —NR^{a2}R^{a3}, —CN, —C(O)—OR^{a1}, —SO₂—R^{a1}, C₁₋₄ alkyl, C₃₋₆ cycloalkyl, or 4 to 7 membered monocyclic heterocyclyl, wherein the C₁₋₄ alkyl and C₃₋₆ cycloalkyl represented by R⁹ are each optionally substituted by one to three substituents independently selected from halo and C₁₋₃ alkoxy; and R^{a1}, R^{a2} and R^{a3} are each independently H or C₁₋₃ alkyl.

57. The compound of claim 55, or a pharmaceutically acceptable salt thereof, wherein each R⁹ is independently selected from F, OH, —OCH₃, —OCH₂CH₃, —OCH(CH₃)₂, —OCHF₂, —OCF₃, —OCD₃, —O-cyclopropyl, —NHCH₃, —NH₂, —N(CH₃)₂, —N(CD₃)₂, —N(CH₂CH₃)₂, —N(CH₃)(CH₂CH₃), —NHCH(CH₃)₂, —NHCH₂CH₃, —CN, —CH₃, —CH₂F, —CF₃, —C(O)—OCH₂CH₃, —SO₂—CH₃, —CH₂—OCH₃, —CH₂—CH₂—OCH₃, —CH₂—CH₃, —CH₂—CN, pyrrolidinyl, morpholinyl, azetidinyl, cyclopropyl, —CHF₂, —CH₂—CF₃,

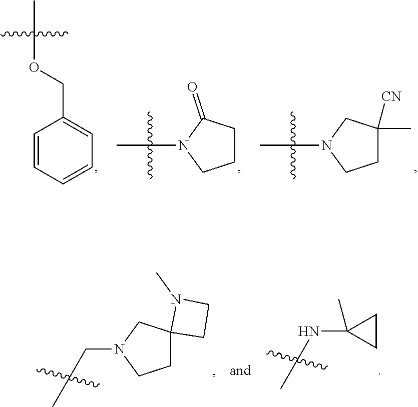

58. The compound of claim 54, or a pharmaceutically acceptable salt thereof, wherein each R⁹ is independently selected from F, OH, —OCH₃, —OCHF₂, —OCF₃, —N(CH₃)₂, —CN, —CH₃, —CF₃, —C(O)—OCH₂CH₃, —SO₂—CH₃, —CH₂—OCH₃, —CH₂—CH₂—OCH₃, —CH₂—CH₃, cyclopropyl, —CHF₂ and —CH₂—CF₃.

59. The compound of claim 56, or a pharmaceutically acceptable salt thereof, wherein each R⁹ is independently selected from F, OH, —OCH₃, —OCHF₂, —OCF₃, —NHCH₃, —NH₂, —N(CH₃)₂, —N(CH₂CH₃)₂, —N(CH₃)(CH₂CH₃), —NHCH(CH₃)₂, —CN, —CH₃, —CF₃, —C(O)—OCH₂CH₃, —SO₂—CH₃, —CH₂—OCH₃, —CH₂—CH₂—OCH₃, —CH₂—CH₃, pyrrolidinyl, morpholinyl, cyclopropyl, —CHF₂, and —CH₂—CF₃.

60. The compound of any one of claims 1-53, or a pharmaceutically acceptable salt thereof, wherein two of R⁹, taken together with their intervening atoms, form a 4 to 6 membered monocyclic heterocyclyl optionally substituted by one to two substituents independently selected from halo, C₁₋₄ alkyl and C₁₋₄ haloalkyl.

61. The compound of any one of claims 1-40, or a pharmaceutically acceptable salt thereof, wherein R³ is selected from H, Cl, —CN, —CH₃, —CH₂F, —CHF₂, —CF₃, —CH₂—CH₃, —CH₂CF₃, —CHF—CH₃, —CH(CH₃)₂, —CH(CF₃)₂, —CHF—CH₂F, —CH₂—CH₂—CN, —CH₂—CH₂—CH₃, —CH(CH₃)—CH₂—CH₃, —CH₂—CH(CH₃)₂, —CH₂-cyclopropyl, —CH₂-morpholinyl, —CH₂OH, —CH₂—OCH₃, —C(CH₃)—CH₂—CH₃, —CH(CH₃)—CF₃, —CH(CH₃)—OCH₃, —CH₂N(CH₃)₂, —CH═CH₂, —NH₂, —NHCH₃, —NHCH₂CH₃, —N(CH₃)₂, —OCH₃, —N(CH₃)—CH₂—CH₂—OCH₃, —N(CH₃)CH₂C(CH₃)₂OCH₃, —C(O)—OCH₃, —C(O)—OCH₂CH₃, —SO₂—CH₃, —O—CH(CH₃)₂, —O—CH₂—CH₂—OCH₃, 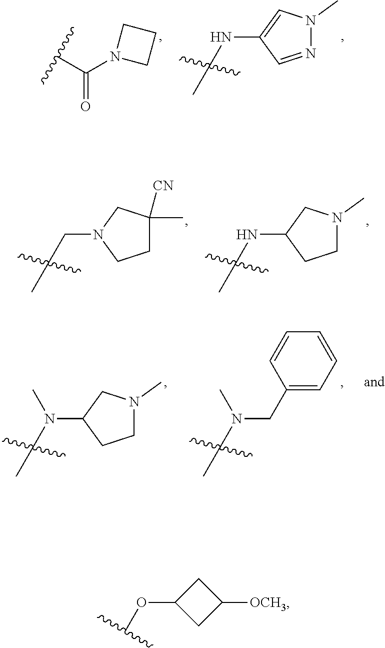
wherein 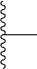
represents a bond to ring B.

62. The compound of any one of claims 1-40, or a pharmaceutically acceptable salt thereof, wherein R³ is selected from H, Cl, —CH₃, —CH₂F, —CHF₂, —CF₃, —CH₂—CH₃, —CHF—CH₃, —CH(CH₃)₂, —CHF—CH₂F, —CH₂—CH₂—CN, —CH₂—CH₂—CH₃, —CH₂—CH(CH₃)₂, —CH₂— cyclopropyl, —CH₂—OCH₃, —CH(CH₃)—CH₂—CH₃, —CH(CH₃)—CF₃, —CH(CH₃)—OCH₃, —N(CH₃)₂, —OCH₃, —N(CH₃)—CH₂—CH₂—OCH₃, —C(O)—OCH₃, —SO₂—CH₃, —O—CH(CH₃)₂, —O—CH₂—CH₂—OCH₃, 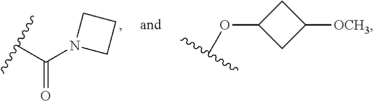 [SMILES: *C(=O)N1CCC1 ; *OC1CC(OC)C1]
wherein 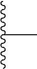
represents a bond to ring B.

63. The compound of any one of claims 1-62, or a pharmaceutically acceptable salt thereof, wherein each R⁷ is independently C₁₋₆ alkyl, C₃₋₆ cycloalkyl or 4 to 6 membered monocyclic heterocyclyl, wherein the C₁₋₆ alkyl, C₃₋₆ cycloalkyl and 4 to 6 membered monocyclic heterocyclyl represented by R⁷ are each optionally substituted by one to three substituents independently selected from halo, C₁₋₃alkyl and C₁₋₃ haloalkyl.

64. The compound of claim 63, or a pharmaceutically acceptable salt thereof, wherein each R⁷ is independently selected from:
—CH₃,

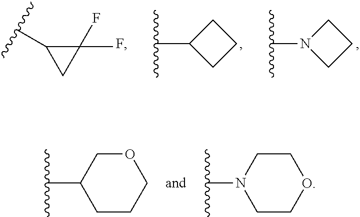

65. The compound of any one of claims 1-64, or a pharmaceutically acceptable salt thereof, wherein:
each R^{O1} is independently H, C₁₋₆ alkyl, 3 to 6 membered monocyclic or bicyclic carbocyclyl, 4 to 6 membered monocyclic heterocyclyl, or 6 membered heteroaryl, wherein the C₁₋₆ alkyl, 3 to 6 membered monocyclic or bicyclic carbocyclyl, 4 to 6 membered monocyclic heterocyclyl and 6 membered heteroaryl represented by R^{O1} are each optionally substituted by one to three R^{O2}; and
each R^{O2} is independently halo, OH, —CN, C₁₋₄ alkoxy, C₁₋₄ alkyl, 3 to 5 membered monocyclic carbocyclyl, 4 to 7 membered monocyclic or bicyclic heterocyclyl or phenyl, wherein the C₁₋₄ alkyl, 3 to 5 membered monocyclic carbocyclyl, 4 to 7 membered monocyclic or bicyclic heterocyclyl and phenyl are each optionally substituted with C₁₋₃ alkoxy, C₁₋₃ haloalkoxy, or one to three halo.

66. The compound of claim 65, or a pharmaceutically acceptable salt thereof, wherein the 4 to 7 membered monocyclic or bicyclic heterocyclyl or 6 membered heteroaryl represented by R^{O1} or R^{O2} are each independently selected from morpholino, oxetanyl, pyridinyl, pyrimidinyl, tetrahydrofuranyl, tetrahydropyranyl, morpholinyl, azetidinyl, oxaspiro[2.4]heptane, pyrrolidinyl and piperidinyl.

67. The compound of any one of claims 1-66, or a pharmaceutically acceptable salt thereof, wherein each R^{O2} is independently selected from F, —CN, OH, —OCH₃, —CH₃, —CHF₂, —CF₃, 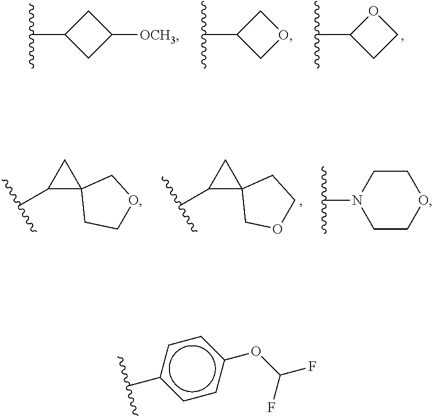
wherein 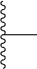
represents a bond to R^{O1}.

68. The compound of any one of claims 1-66, or a pharmaceutically acceptable salt thereof, wherein each R^{O1} is independently selected from H, —CH₃, —CHF₂, —CF₃, —CH₂—CH₃, —CH₂—CHF₂, —CH₂—CF₃, —CH₂—CH₂—CH₃, —CH(CH₃)₂, —CH(CF₃)₂, —CH₂—CH₂—OCH₃, —CH(CH₃)—CH₂—OCH₃, —CH₂—CH(CH₃)—OCH₃,

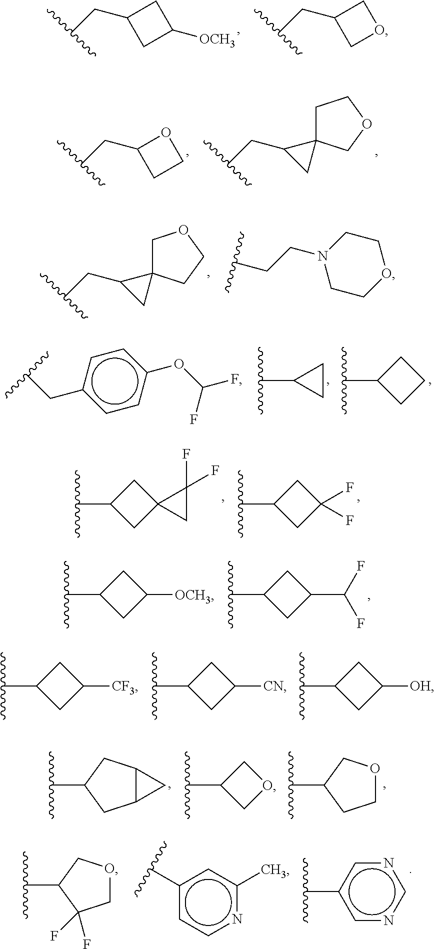

69. The compound of any one of claims 1-68, or a pharmaceutically acceptable salt thereof, wherein each R^{O3} is independently C₁₋₆ alkyl optionally substituted by one to three substituents independently selected from halo and C₁₋₄ haloalkyl.

70. The compound of any one of claims 1-68, or a pharmaceutically acceptable salt thereof, wherein each R^{O3} is independently —CH₃, —CH₂CH₃ or —C(CH₃)₃.

71. The compound of any one of claims 1-70, or a pharmaceutically acceptable salt thereof, wherein R^{O4} is C₁₋₄ alkyl or C₃₋₆ cycloalkyl, each optionally substituted by one to three substituents independently selected from halo and C₁₋₃ alkoxy.

72. The compound of any one of claims 1-70, or a pharmaceutically acceptable salt thereof, wherein R^{O4} is selected from —CH₃, —CH₂—CH₃, —CH(CH₃)₂, —CH₂—CH₂—OCH₃, and

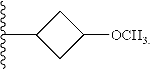 [SMILES: *C1CC(OC)C1]

73. The compound of any one of claims 1-72, or a pharmaceutically acceptable salt thereof, wherein R^{N1} and R^{N2} each independently is H, C₃₋₆ cycloalkyl or C₁₋₄ alkyl optionally substituted with C₁₋₃ alkoxy.

74. The compound of any one of claims 1-72, or a pharmaceutically acceptable salt thereof, wherein R^{N1} and R^{N2} each independently represent H, —CH₃, —CH(CH₃)₂, —CH₂—CH₂—OCH₃ or cyclohexyl.

75. The compound of any one of claims 1-74, or a pharmaceutically acceptable salt thereof, wherein R^{N3} is H.

76. The compound of any one of claims 1-75, or a pharmaceutically acceptable salt thereof, wherein R^{N4} is H.

77. The compound of any one of claims 1-76, or a pharmaceutically acceptable salt thereof, wherein R⁴ is H or —CH₃.

78. The compound of claim 77, or a pharmaceutically acceptable salt thereof, wherein R⁴ is H.

79. The compound of claim 1, wherein the compound is represented by formula (VII′) or (VIII′): 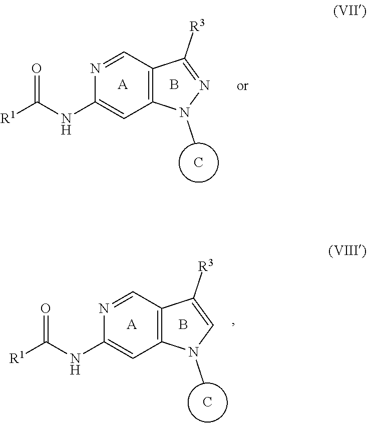
or a pharmaceutically acceptable salt thereof, wherein:
ring C is 6 membered heteroaryl optionally substituted by one to three R^{C};
each R^{C} is independently —OR^{O1}, C₁₋₄ alkyl or 5 membered heterocyclyl, wherein the C₁₋₄ alkyl and 5 membered heterocyclyl represented by R^{C} are each optionally substituted with one to three R^{C1};
each R^{C1} is independently halo or —OR^{O1};
R¹ is H or —CH₃;
R³ is H, C₁₋₄ alkyl, C₃₋₅ cycloalkyl, or 5 to 7 membered monocyclic or bicyclic heterocyclyl, wherein the C₁₋₄ alkyl, C₃₋₅ cycloalkyl and 5 to 7 membered monocyclic or bicyclic heterocyclyl represented by R³ are each optionally substituted by one to three R⁹;
each R⁹ is independently —OR^{O1}, —NR^{N1}R^{N2}, 5 to 7 membered monocyclic heterocyclyl, or C₁₋₄ alkyl;
each R^{O1} is independently H, C₁₋₄ alkyl or C₃₋₅ cycloalkyl, wherein the C₁₋₄ alkyl and C₃₋₅ cycloalkyl are each optionally substituted by R^{O2};
each R^{O2} is independently —CN or C₁₋₄ alkoxy;
R^{N1} and R^{N2} are each independently H or C₁₋₃ alkyl.

80. The compound of claim 1, wherein the compound is represented by formula (VII) or (VIII): 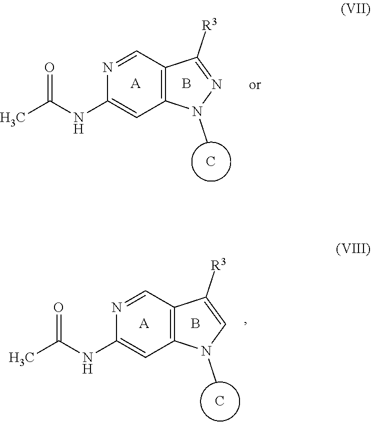
or a pharmaceutically acceptable salt thereof, wherein:
ring C is 6 membered heteroaryl optionally substituted by one to three R^{C};
each R^{C} is independently —OR^{O1}, C₁₋₄ alkyl or 5 membered heterocyclyl, wherein the C₁₋₄ alkyl and 5 membered heterocyclyl represented by R^{C} are each optionally substituted with one to three R^{C1};
each R^{C1} is independently halo or —OR^{O1};
R³ is H, C₁₋₄ alkyl, C₃₋₅ cycloalkyl, or 5 to 7 membered monocyclic or bicyclic heterocyclyl, wherein the C₁₋₄ alkyl, C₃₋₅ cycloalkyl and 5 to 7 membered monocyclic or bicyclic heterocyclyl represented by R³ are each optionally substituted by one to three R⁹;
each R⁹ is independently —OR^{O1}, —NR^{N1}R^{N2}, 5 to 7 membered monocyclic heterocyclyl, or C₁₋₄ alkyl;
each R^{O1} is independently H, C₁₋₄ alkyl or C₃₋₅ cycloalkyl, wherein the C₁₋₄ alkyl and C₃₋₅ cycloalkyl are each optionally substituted by R^{O2};
each R^{O2} is independently —CN or C₁₋₄ alkoxy;
R^{N1} and R^{N2} are each independently H or C₁₋₃ alkyl.

81. The compound of claim 79 or 80, wherein each R⁹ is independently —OR^{O1}, —NR^{N1}R^{N2}, or C₁₋₄ alkyl.

82. The compound of claim 79 or 80, or a pharmaceutically acceptable salt thereof, wherein ring C is pyridinyl, pyrazinyl or pyrimidinyl, each of which is optionally substituted by one or two R^{C}.

83. The compound of claim 79 or 80, or a pharmaceutically acceptable salt thereof, wherein ring C is pyrimidinyl, pyrazinyl or thiazolyl, each of which is optionally substituted by one or two R^{C}.

84. The compound of claim 79 or 80, or a pharmaceutically acceptable salt thereof, wherein ring C is selected from: 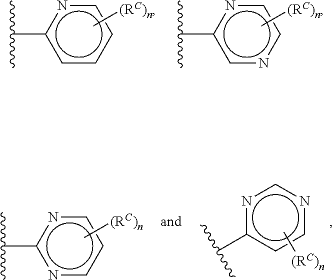
and wherein 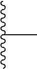
represents a bond to ring B, and n is 0, 1, or 2.

85. The compound of claim 79 or 80, or a pharmaceutically acceptable salt thereof, wherein ring C is selected from: 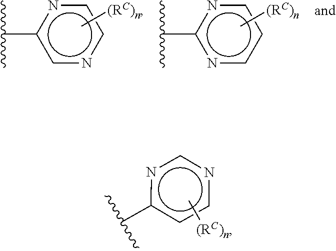
and wherein 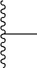
represents a bond to ring B, and n is 0, 1, or 2.

86. The compound of claim 79 or 80, or a pharmaceutically acceptable salt thereof, wherein ring C is selected from: 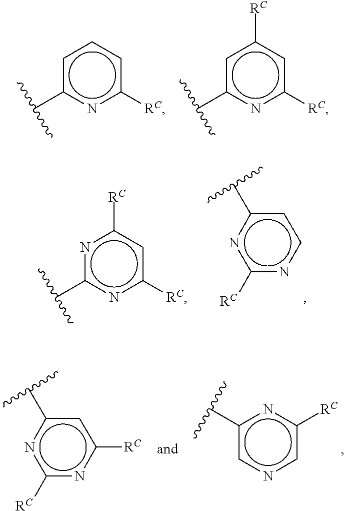
and wherein 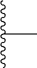
represents a bond to ring B, and two R^{C} groups in ring C may be the same or different.

87. The compound of claim 79 or 80, or a pharmaceutically acceptable salt thereof, wherein ring C is selected from: 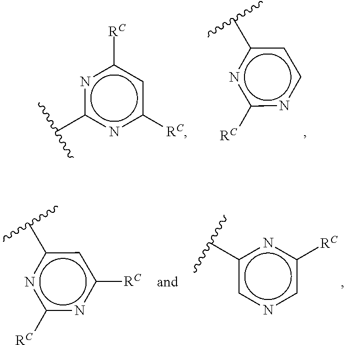
and wherein 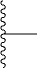
represents a bond to ring B, and two R^{C} groups in ring C may be the same or different.

88. The compound of any one of claims 79-87, or a pharmaceutically acceptable salt thereof, wherein each R^{C} is independently —OR^{O1}, C₁₋₂alkyl, C₁₋₂haloalkyl, or 5 membered oxygen-containing heterocyclyl optionally substituted with one R^{C1}.

89. The compound of any one of claims 79-88, or a pharmaceutically acceptable salt thereof, wherein R^{C} is tetrahydrofuranyl optionally substituted with one R^{C1}.

90. The compound of any one of claims 79-89, or a pharmaceutically acceptable salt thereof, wherein R^{C} is 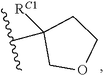
wherein 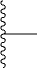
represents a bond ring C.

91. The compound of any one of claims 79-90, or a pharmaceutically acceptable salt thereof, wherein each R^{C1} is independently F or —OCH₃.

92. The compound of any one of claims 79-88, or a pharmaceutically acceptable salt thereof, wherein each R^{C} is independently selected from —CH₃, —CH₂CH₃, —CF₂CH₃, —CF(CH₃)₂, —OCH₃, —O—CH₂—CH₂—O—CH₃, 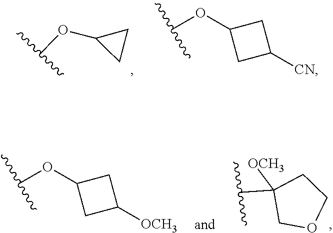
wherein 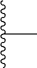
represents a bond to ring C.

93. The compound of any one of claims 79-88, or a pharmaceutically acceptable salt thereof, wherein each R^{C} is independently selected from —CH₃, —CF₂CH₃, —OCH₃, —O—CH₂—CH₂—O—CH₃, 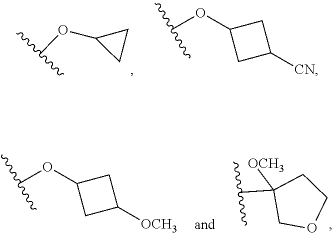
wherein 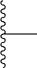
represents a bond to ring C.

94. The compound of any one of claims 79-88, or a pharmaceutically acceptable salt thereof, wherein each R^{C} is independently selected from —CH₃, —CH₂CH₃, —CF₂CH₃, —CF(CH₃)₂—OCH₃, —O—CH₂—CH₂—O—CH₃, 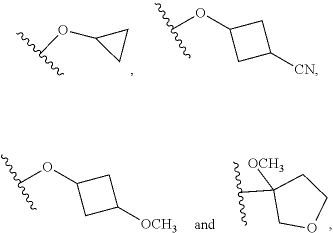
wherein 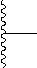
represents a bond to ring C.

95. The compound of any one of claims 79-94, or a pharmaceutically acceptable salt thereof, wherein the 5 to 7 membered monocyclic or bicyclic heterocyclyl represented by R³ is selected from 3,6-diazabicyclo[3.1.1]heptanyl, 2-oxa-5-azabicylo [2.2.1]heptanyl, 6-oxa-3-azabicyclo [3.1.1]heptanyl, piperazinyl, and pyrrolidinyl, each of which is optionally substituted by one or two R⁹.

96. The compound of any one of claims 79-94, or a pharmaceutically acceptable salt thereof, wherein R³ is selected from H, —CH₂CH₃, 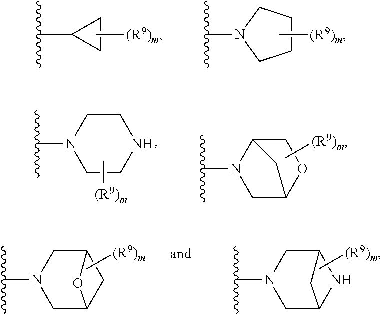
wherein 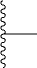
represents a bond to ring B, and m is 0, 1 or 2.

97. The compound of any one of claims 79-94, or a pharmaceutically acceptable salt thereof, wherein R³ is selected from H, —CH₂CH₃, 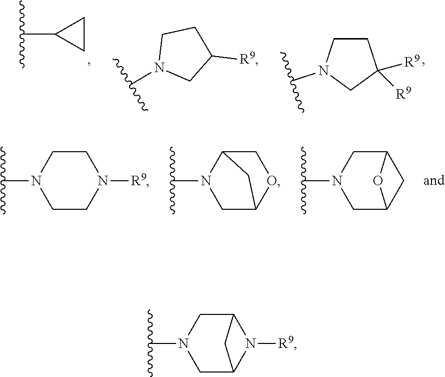
wherein 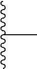
represents a bond to ring B.

98. The compound of claim any one of claims 79-97, or a pharmaceutically acceptable salt thereof, wherein each R⁹ is independently selected from —OH, —OCH₃, —N(CH₃)₂, and —CH₃.

99. The compound of claim any one of claims 79-97, or a pharmaceutically acceptable salt thereof, wherein each R⁹ is independently selected from —OH, —OCH₃, —NH₂, —NHCH₃, —N(CH₃)₂, —N(CH₃)(CH₂CH₃), —N(CH₂CH₃)₂, pyrrolidinyl, morpholinyl, —CH₂CH₃, and —CH₃.

100. The compound of any one of claims 79-99, or a pharmaceutically acceptable salt thereof, wherein R^{O1} are each independently selected from H, —CH₃, —CH₂CH₂OCH₃, cyclopropyl,

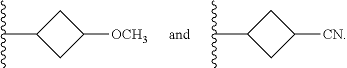 [SMILES: *C1CC(OC)C1 ; *C1CC(C#N)C1]

101. The compound of any one of claims 58 and 95-99, or a pharmaceutically acceptable salt thereof, wherein:
ring C is selected from the following: 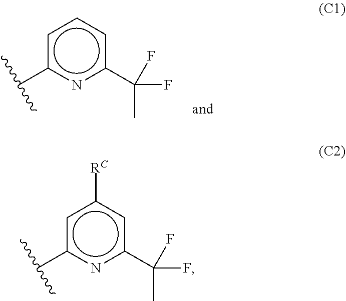
and
R^{C} is C₁₋₃alkyl or C₁₋₃alkoxy;
R³ is selected from pyrrolidinyl, morphonlinyl, 6-oxa-3-azabicyclo[3.1.1]heptanyl, and 3,6-diazabicyclo[3.1.1]heptanyl, each of which is optionally substituted by one to two R⁹;
R⁹ is independently halo, C₁₋₄ alkyl, OH, —OC₁₋₄alkyl, or —NR^{a2}R^{a3}; and
R^{a2} and R^{a3} are each independently H or C₁₋₃alkyl.

102. The compound of any one of claims 79, 80, and 95-99, or a pharmaceutically acceptable salt thereof, wherein:
ring C is selected from the following: 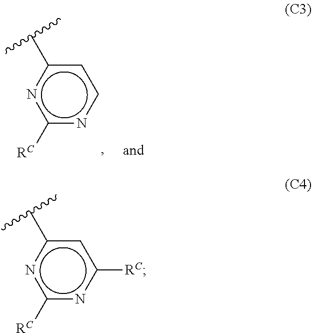
R^{C}, for each occurrence, is independently C₁₋₃alkyl, C₁₋₃haloalkyl or C₁₋₃alkoxy;
R³ is selected from pyrrolidinyl, morphonlinyl, 6-oxa-3-azabicyclo[3.1.1]heptanyl, and 3,6-diazabicyclo[3.1.1]heptanyl, each of which is optionally substituted by one to two R⁹;
R⁹ is independently halo, C₁₋₄ alkyl, OH, —OC₁₋₄alkyl, or —NR^{a2}R^{a3}; and
R^{a2} and R^{a3} are each independently H or C₁₋₃alkyl.

103. The compound of claim 101 or 102, or a pharmaceutically acceptable salt thereof, wherein:
R^{C} in formula (C2) is C₁₋₂alkyl or C₁₋₂alkoxy;
R^{C} in formula (C3) is C₁₋₂haloalkyl; and
for formula (C4), one of R^{C} is C₁₋₂haloalkyl and the other is C₁₋₃alkyl or C₁₋₃alkoxy.

104. The compound of any one of claims 101-103, or a pharmaceutically acceptable salt thereof, wherein R³ is selected from: 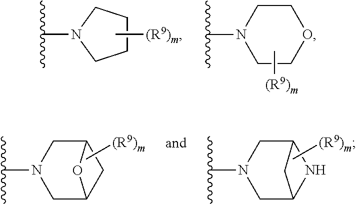
and R⁹ is C₁₋₃alkyl, —OC₁₋₃alkyl or —NR^{a2}R^{a3}; and m is 0, 1 or 2.

105. The compound of any one of claims 101-104, or a pharmaceutically acceptable salt thereof, wherein:
R^{C} in formula (C3) is —CF₂CH₃, and for formula (C4), one of R^{C} is —CF₂CH₃ and the other R^{C} is C₁₋₃alkyl or C₁₋₃alkoxy;
m is 1 or 2; and
R⁹ for each occurrence is independently C₁₋₃alkyl, —OC₁₋₃alkyl or —NR^{a2}R^{a3}.

106. The compound of any one of claims 101-104, or a pharmaceutically acceptable salt thereof, wherein:
Ring C is 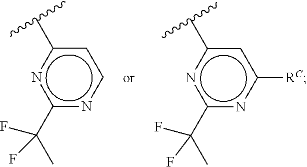
R^{C} is C₁₋₂alkyl or C₁₋₂alkoxy;
R³ is 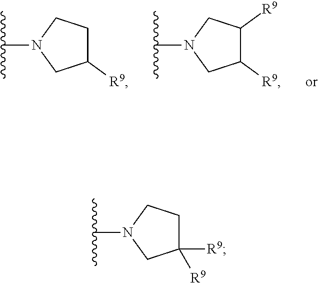
R⁹ for each occurrence, is independently C₁₋₂alkyl or —NR^{a2}R^{a3}; and
R^{a2} and R^{a3} are C₁₋₂alkyl.

107. The compound of claim 106, or a pharmaceutically acceptable salt thereof, wherein R^{C} is —CH₃, —CH₂CH₃ or —OCH₃

108. The compound of any one of claims 101-107, or a pharmaceutically acceptable salt thereof, wherein R⁹ is —CH₃ or —N(CH₃)₂.

109. The compound of claim 1, wherein the compound is represented by formula (VIIA′): 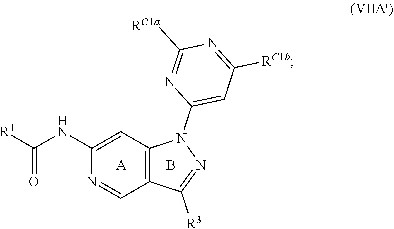
or a pharmaceutically acceptable salt thereof, wherein:
R¹ is H or CH₃;
R^{C1a} is C₁₋₃alkyl substituted with 1 to 3 halo;
R^{C1b} is C₁₋₃alkyl;
R³ is selected from 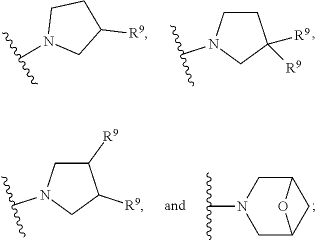
each R⁹ is independently —NR^{N1}R^{N2}, C₁₋₄ alkyl, morpholinyl, or pyrrolidinyl; and
R^{N1} and R^{N2} are each independently H or C₁₋₃ alkyl.

110. The compound of claim 1, or a pharmaceutically acceptable salt, wherein the compound is represented by formula (VIIA): 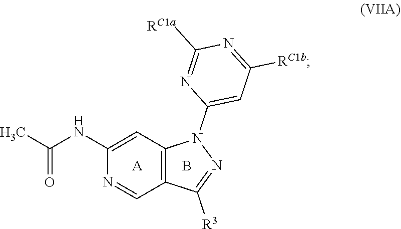
or a pharmaceutically acceptable salt thereof, wherein:
R^{C1a} is C₁₋₃alkyl substituted with 1 to 3 halo;
R^{C1b} is C₁₋₃alkyl;
R³ is selected from 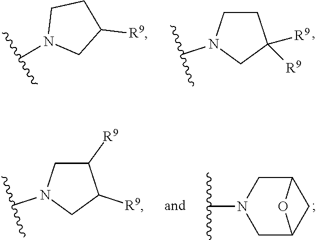
each R⁹ is independently —NR^{N1}R^{N2}, C₁₋₄ alkyl, morpholinyl, or pyrrolidinyl; and
R^{N1} and R^{N2} are each independently H or C₁₋₃ alkyl.

111. The compound of claim 109 or 110, or a pharmaceutically acceptable salt thereof, wherein R^{C1a} is —CF₂CH₃ and R^{C1b} is —CH₃ or CH₂CH₃.

112. The compound of any one of claims 109-111, or a pharmaceutically acceptable salt thereof, wherein each R⁹ is independently —NH₂, —NHCH₃, —N(CH₃)₂, —N(CH₃)(CH₂CH₃), —N(CH₂CH₃)₂, 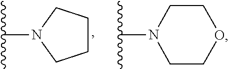
—CH₃ or —CH₂CH₃.

113. The compound of claim 1, wherein the compound is represented by the following formula: 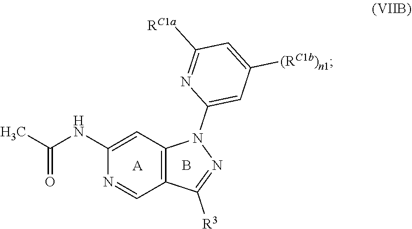
or a pharmaceutically acceptable salt thereof, wherein:
R^{C1a} is C₁₋₃alkyl substituted with 1 to 3 halo;
R^{C1b} is C₁₋₃alkoxy;
n1 is 0 or 1
R³ is 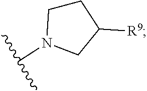
R⁹ is —NR^{N1}R^{N2};
R^{N1} and R^{N2} are each independently H or C₁₋₃ alkyl.

114. The compound of claim 113, or a pharmaceutically acceptable salt thereof, wherein R^{C1a} is —CF₂CH₃ or —CF(CH₃)₂.

115. The compound of claim 113, or a pharmaceutically acceptable salt thereof, wherein n1 is 0 or n1 is 1 and R^{C1b} is —OCH₃.

116. The compound of any one of claims 113-115, or a pharmaceutically acceptable salt thereof, wherein R⁹ is —NHCH(CH₃)₂.

117. A pharmaceutical composition comprising a compound according to any one of claims 1-116, or a pharmaceutically acceptable salt thereof, and a pharmaceutically acceptable carrier.

118. A method of inhibiting tyrosine kinase 2 (TYK2) activity in a subject in need thereof comprising administering to the subject an effective amount of a compound according to any one of claims 1-116 or a pharmaceutically acceptable salt thereof or a pharmaceutical composition according to claim 117.

119. A method of treating a disease or disorder responsive to inhibition of tyrosine kinase 2 (TYK2) in a subject comprising administering to the subject an effective amount of a compound according to any one of claims 1-116 or a pharmaceutically acceptable salt thereof or a pharmaceutical composition according to claim 117.

120. The method of claim 119, wherein the disease or disorder is inflammation, autoimmune disease, neuroinflammation, arthritis, rheumatoid arthritis, spondyloarthropathies, systemic lupus erythematosus, cutaneous lupus erythematosus, lupus nephritis, arthritis, osteoarthritis, gouty arthritis, pain, fever, pulmonary sarcoisosis, silicosis, cardiovascular disease, atherosclerosis, myocardial infarction, thrombosis, congestive heart failure and cardiac reperfusion injury, cardiomyopathy, stroke, ischaemia, reperfusion injury, brain edema, brain trauma, neurodegeneration, liver disease, inflammatory bowel disease, Crohn's disease, ulcerative colitis, nephritis, retinitis, retinopathy, macular degeneration, glaucoma, diabetes (type 1 and type 2), diabetic neuropathy, viral and bacterial infection, myalgia, endotoxic shock, toxic shock syndrome, osteoporosis, multiple sclerosis, endometriosis, menstrual cramps, vaginitis, candidiasis, cancer, fibrosis, systemic sclerosis, obesity, muscular dystrophy, polymyositis, dermatomyositis, autoimmune hepatitis, primary biliary cirrhosis, primary sclerosing cholangitis, vitiligo, alopecia, Alzheimer's disease, skin flushing, eczema, psoriasis, atopic dermatitis and sunburn.
